(19) United States
(12) Patent Application Publication
(10) Pub. No.: US 2023/0093099 A1
(43) Pub. Date: Mar. 23, 2023

(54) COMPOUNDS AND METHODS OF TREATING CANCERS

(71) Applicant: CULLGEN (SHANGHAI), INC., Shanghai (CN)

(72) Inventors: Michael Bruno PLEWE, San Diego, CA (US); Jialiang Wang, Nashville, TN (US); Xiaoran Han, Shanghai (CN); Liqun Chen, Shanghai (CN)

(21) Appl. No.: 17/600,808

(22) PCT Filed: Apr. 2, 2020
(86) PCT No.: PCT/CN2020/083041

§ 371 Date: Oct. 1, 2021

(30) Foreign Application Priority Data
Apr. 2, 2019 (CN) PCT/CN2019/081068

(51) Int. Cl. A61K 47/54; A61P 35/00

(52) U.S. Cl. CPC A61K 47/545; A61P 35/00; A61K 47/54

(57) ABSTRACT

This disclosure relates to heterobifunctional compounds (e.g., bi-functional small molecule compounds), compositions comprising one or more of the heterobifunctional compounds, and to methods of use the heterobifunctional compounds for the treatment of certain disease in a subject in need thereof. The disclosure also relates to methods for identifying such heterobifunctional compounds.

### BACKGROUND OF THE INVENTION

This disclosure relates to heterobifunctional compounds (e.g., bi-functional small molecule compounds), compositions comprising one or more of the heterobifunctional compounds, and to methods of use of the heterobifunctional compounds for the treatment of certain diseases in a subject in need thereof. The disclosure also relates to methods for identifying such heterobifunctional compounds.

### SUMMARY OF THE INVENTION

According to one aspect of the present disclosure, a heterobifunctional compound disclosed herein comprises a Janus kinase (JAK) ligand conjugated to a degradation tag, or a pharmaceutically acceptable salt or analog thereof.

In one embodiment JAK ligand is capable of binding to a JAK protein comprising JAK1, JAK2, JAK3, and Tyrosine Kinase 2 (TYK2), a JAK mutant, JAK deletion, or a JAK fusion protein.

In one embodiment, the JAK ligand is a JAK inhibitor or a portion of JAK inhibitor.

In another embodiment, the JAK ligand is selected from the group consisting of BSK805 (NVP-BSK805), 1-amino-[1,2,4]triazolo[1,5-a]pyridines (Cmpd 12), TG101209, CEP-33799, Ruxolitinib, Tofacitinib (CP-690550), Baricitinib, Oclacitinib, Cerdulatinib (PRT-062070), Decernotinib (VX509), Delgocitinib (JTE-052), Fedratinib, Filgotinib (GLP0634), Gandotinib (LY2784544), Ilginatinib (NS-018), Itacitinib (INCB03911), Lestauritinib, Momelotinib (CYT387), Pacritinib (SB1578), Peficitinib, Solcitinib (GSK2586184, GLG0778), Upadacitinib (ABT-494), AT9283, AZ-3, AZ960, AZD1480, BMS-986165, BMS-911543, BVB808 (NVP-BVB808), BBT594 (NVP-BBT594), CHZ868, FM381, PF-04965842, PF-06263276, PF-06651600, PF-06700841, SAR-20347, NDI-031301, NDI-31232, NVP-P830, SHR-0302, VR588, XL019, R333 and R348, 3-amido pyrrolopyrazine (Cmpd 3q), pyridone containing tetracycle (Cmpd 6), triazolo-pyrrolopyridines (Cmpd 7), pyrazolopyrimidines (Cmpd 7j), imidazolopyridines (Cmpd 19), 1-methyl-1H-imidazole derivatives (Cmpd 19a), C-2 methyl imidazopyrrolopyridines (Cmpd 20), pyrazolopyridinone (Cmpd 20a), 9H-carbazole-1-carboxamides (Cmpd 21), thianopyridines (Cmpd 23), pyrazole-4-carboxamide (Cmpd 28), imidazopyridine (Cmpd 30), hydroxyethyl imidazo-pyrrolopyridines (Cmpd 31), pyrrolopyridazines (Cmpd 35), 6-oxopyridopyrimidines (Compound 36), 2-aminopyrazolo[1,5-a]pyrimidines (Cmpd 45), cyclopropyl amides (Cmpd 46), imidazo-pyrrolopyridines (Cmpd 49), 1-amino-5H-pyrido[4,3-b]indol-4-carboxamides (Cmpd 65), Cmpd 3, Cmpd 13a, Cmpd 45a, and analogs thereof.

In another embodiment, the degradation tag binds to an ubiquitin ligase or is a hydrophobic group or a tag that leads to misfolding of the JAK proteins.

In another embodiment, the ubiquitin ligase is an E3 ligase.

In another embodiment, the E3 ligase is selected from the group consisting of a cereblon E3 ligase, a VHL E3 ligase, an IAP ligase, a MDM2 ligase, a TRIM24 ligase, a TRIM21 ligase, a KEAP1 ligase, DCAF16 ligase, RNF4 ligase, RNF114 ligase, and AhR ligase.

In another embodiment, the degradation tag is selected from the group consisting of pomalidomide, thalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, CPD36, GDC-0152, CRBN-1, CRBN-2, CRBN-3, CRBN-4, CRBN-5, CRBN-6, CRBN-7, CRBN-8, CRBN-9, CRBN-10, CRBN-11, and analogs thereof.

In another embodiment, the JAK ligand is conjugated to the degradation tag via a linker moiety.

In another embodiment, the JAK ligand comprises a moiety of FORMULA 1:

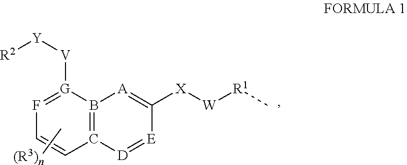

wherein

the linker moiety of the heterobifunctional compound is attached to R¹;

A and D are independently selected from CR⁴ and N, wherein

R⁴ is selected from hydrogen, halogen, optionally substituted C₁-C₈ alkyl, and optionally substituted 3-10 membered carbocyclyl;

B, C and G are independently selected from C and N; with the proviso that at most only one of B, C and G is N;

E and are independently selected from null, CR⁵ and N, wherein

R⁵ is selected from hydrogen, halogen, optionally substituted C₁-C₈ alkyl, and optionally substituted 3-10 membered carbocyclyl;

X and Y are independently selected from null, or a bivalent moiety selected from null, CR⁶R⁷, CO, CO₂, CONR⁶, NR⁶, NR⁶CO, NR⁶CO₂, NR⁶C(O)NR⁷, NR⁶SO, NR⁶SO₂, NR⁶SO₂NR⁷, O, OC(O), OCO₂, OCONR⁶, S, SO, SO₂, and SO₂NR⁶, wherein

R⁶ and R⁷ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkylamino, optionally substituted 3-10 membered carbocyclyl, optionally substituted 4-10 membered heterocyclyl, optionally substituted aryl, and optionally substituted heteroaryl, or

R⁶ and R⁷ together with the atom or atoms to which they are connected form a 3-20 membered carbocyclyl ring or 4-20 membered heterocyclyl ring;

V and W are independently selected from null, carbocyclyl, heterocyclyl, aryl, and heteroaryl, which are optionally substituted with one or more substituents independently selected from hydrogen, halogen, oxo, CN, NO₂, OR⁸, SR⁸, NR⁸R⁹, OCOR⁸, OCO₂R⁸, OCON(R⁸)R⁹, COR⁸, CO₂R⁸, CON(R⁸)R⁹, SOR⁸, SO₂R⁸, SO₂N(R⁸)R⁹, NR¹⁰CO₂R⁸, NR¹⁰COR⁸, NR¹⁰C(O)N(R⁸)R⁹, NR¹⁰SOR⁸, NR¹⁰SO₂R⁸, NR¹⁰SO₂N(R⁸)R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring;

R¹ is connected to the “linker” moiety of the heterobifunctional compound, and is selected from null, R′—R″, R′OR″, R′SR″, R′N(R¹¹)R″, R′OC(O)R″, R′OC(O)OR″, R′OCON(R¹¹)R″, R′C(O)R″, R′C(O)OR″, R′CON(R¹¹)R″, R′S(O)R″, R′S(O)₂R″, R′SO₂N(R¹¹)R″, R′NR¹²C(O)OR″, R′NR¹²C(O)R″, R′NR¹²C(O)N(R¹¹)R″, R′NR¹²S(O)R″, R′NR¹²S(O)₂R″, and R′NR¹²S(O)₂NR¹¹R″, wherein

R′ and R″ are independently selected from null, optionally substituted C₁-C₈ alkyl, optionally substituted C₂-C₈ alkenyl, optionally substituted C₂-C₈ alkynyl, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused carbocyclyl, optionally substituted C₄-C₁₃ fused heterocyclyl, optionally substituted C₃-C₁₃ bridged carbocyclyl, optionally substituted C₄-C₁₃ bridged heterocyclyl, optionally substituted C₃-C₁₃ spiro carbocyclyl, optionally substituted C₄-C₁₃ spiro heterocyclyl, optionally substituted aryl, and optionally substituted heteroaryl;

R¹¹ and R¹² are independently selected from optionally substituted C₁-C₈ alkyl, optionally substituted C₂-C₈ alkenyl, optionally substituted C₂-C₈ alkynyl, optionally substituted 3-10 membered carbocyclyl, optionally substituted 4-10 membered heterocyclyl, optionally substituted aryl, and optionally substituted heteroaryl; or

R′ and R″, R¹¹ and R¹², R′ and R¹¹, R′ and R¹², R″ and R¹¹, R″ and R¹², together with the atom to which they are connected, form a 3-20 membered carbocyclyl or 4-20 membered heterocyclyl ring;

R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted 3-10 membered carbocyclyl, optionally substituted 4-10 membered heterocyclyl, optionally substituted aryl, and optionally substituted heteroaryl;

R³, at each occurrence, is selected from hydrogen, halogen, optionally substituted C₁-C₈ alkyl, and optionally substituted 3-10 membered carbocyclyl; and

n is selected from 1 or 2.

In one refinement, V is Ar².

In one refinement, the JAK ligand comprises a moiety of FORMULA 1A:

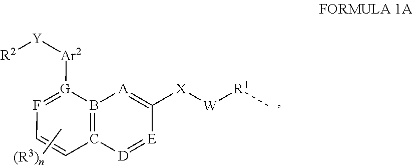

wherein

the linker moiety of the heterobifunctional compound is attached to R¹;

A, B, C, D, E, F, G, X, Y, W, R¹, R², R³, and n are the same as defined in FORMULA 1; and

Ar² is selected from null, aryl and heteroaryl (preferably Ar² is selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, halogen, oxo, CN, NO₂, OR⁸, SR⁸, N(R⁸)R⁹, OCOR⁸, OCO₂R⁸, OCON(R⁸)R⁹, COR⁸, CO₂R⁸, CON(R⁸)R⁹, SOR⁸, SO₂R⁸, SO₂N(R⁸)R⁹, NR¹⁰CO₂R⁸, NR¹⁰COR⁸, NR¹⁰C(O)N(R⁸)R⁹, NR¹⁰SOR⁸, NR¹⁰SO₂R⁸, NR¹⁰SO₂N(R⁸)R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring.

In another refinement, V is Ar²; and W is Ar¹. The JAK ligand comprises a moiety of FORMULA 1B:

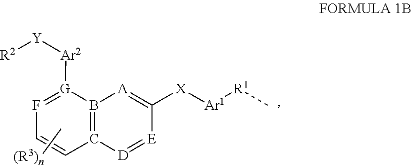

wherein

the linker moiety of the heterobifunctional compound is attached to R¹;

A, B, C, D, E, F, G, X, Y, R¹, R², R³, and n are the same as defined in FORMULA 1; and

Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, halogen, oxo, CN, NO₂, OR⁸, SR⁸, N(R⁸)R⁹, OCOR⁸, OCO₂R⁸, OCON(R⁸)R⁹, COR⁸, CO₂R⁸, CON(R⁸)R⁹, SOR⁸, SO₂R⁸, SO₂N(R⁸)R⁹, NR¹⁰CO₂R⁸, NR¹⁰COR⁸, NR¹⁰C(O)N(R⁸)R⁹, NR¹⁰SOR⁸, NR¹⁰SO₂R⁸, NR¹⁰SO₂N(R⁸)R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring.

In another refinement, A is N.

In another refinement, the JAK ligand comprises a moiety of FORMULA 1C:

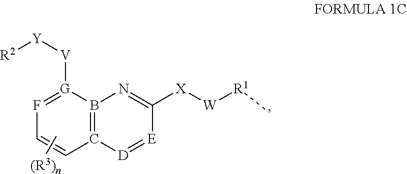

wherein

the linker moiety of the heterobifunctional compound is attached to R¹; and

B, C, D, E, F, G, V, W, X, Y, R¹, R², R³, and n are the same as defined in FORMULA 1.

In another refinement, A is N; and V is Ar².

In another refinement, the JAK ligand comprises a moiety of FORMULA 1D:

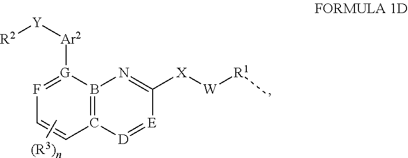

wherein

the linker moiety of the heterobifunctional compound is attached to R¹;

B, C, D, E, F, G, W, X, Y, R¹, R², R³, and n are the same as defined in FORMULA 1; and

Ar² is the same as defined in FORMULA 1A.

In another refinement, A is N; V is Ar²; and W is Ar¹.

In another refinement, the JAK ligand comprises a moiety of FORMULA 1E:

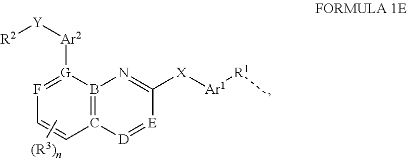

wherein

the linker moiety of the heterobifunctional compound is attached to R¹;

B, C, D, E, F, G, X, Y, R¹, R², R³, and n are the same as defined in FORMULA 1, and

Ar¹ and Ar² are the same as defined in FORMULA 1B.

In another refinement, the JAK ligand comprises a moiety of FORMULAE 1F, 1G, 1H, or 1I:

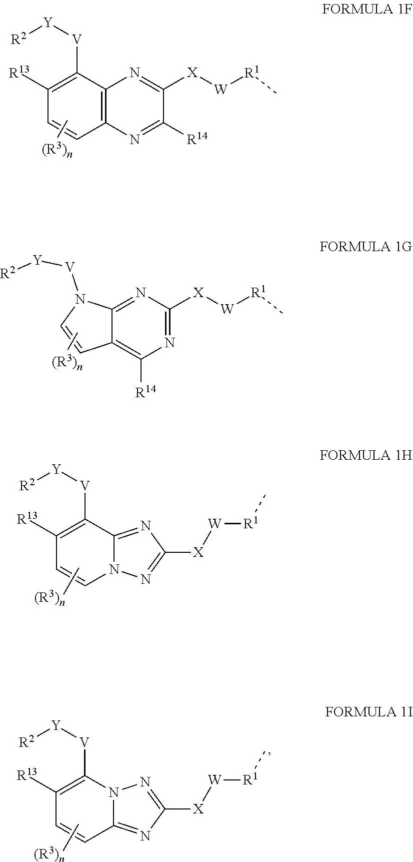

wherein

the linker moiety of the heterobifunctional compound is attached to R¹;

V, W, X, Y, R¹, R², R³, and n are the same as defined in FORMULA 1; and

R¹³ and R¹⁴ are selected from hydrogen, halogen, optionally substituted C₁-C₈ alkyl, and optionally substituted 3-10 membered carbocyclyl.

In another refinement, the JAK ligand comprises a moiety of FORMULAE 1J, 1K, 1L, or 1M:

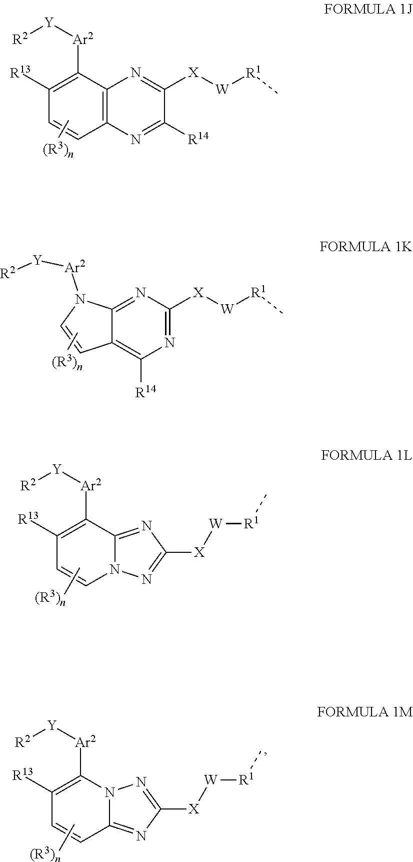

wherein

the linker moiety of the heterobifunctional compound is attached to R¹;

W, X, Y, R¹, R², R³, and n are the same as defined in FORMULA 1;

Ar² is the same as defined in FORMULA 1A; and

R¹³ and R¹⁴ are the same as defined in FORMULAE 1F, 1G, 1H or 1I.

In another refinement, the JAK ligand comprises a moiety of FORMULAE 1N, 1O, 1P, and 1Q:

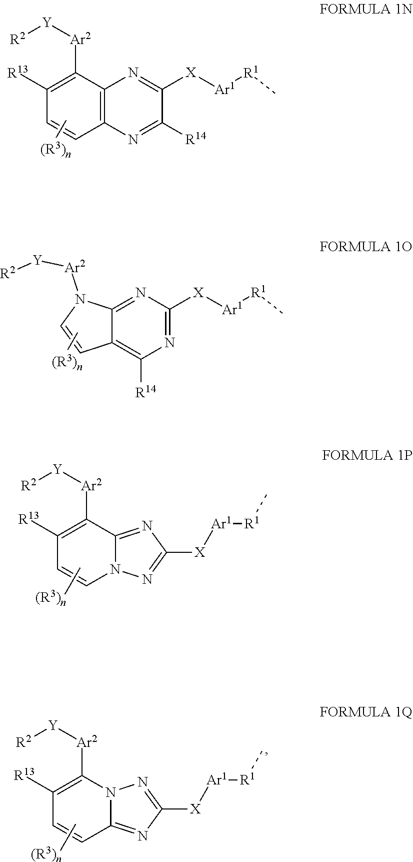

wherein

the linker moiety of the heterobifunctional compound is attached to R¹;

X, Y, R¹, R², R³, and n are the same as defined in FORMULA 1;

Ar¹ and Ar² are the same as defined in FORMULA 1B; and

R¹³ and R¹⁴ are the same as defined in FORMULA 1F, 1G, 1H or 1I.

In another refinement, X is selected from null, O, and NR⁶, wherein

R⁶ is selected from hydrogen, optionally substituted C₁-C₈ alkyl, and optionally substituted 3-10 membered carbocyclyl.

In another refinement, X is selected from null and NH.

In another refinement, Y is selected from null, CR^{6′}R⁷, CO, CO₂, O, SO, SO₂, and NR^{6′}, wherein

R^{6′} and R⁷ are independently selected from hydrogen, optionally substituted C₁-C₈ alkyl, optionally substituted 3-10 membered carbocyclyl, and optionally substituted 3-10 membered heterocyclyl.

In another refinement, Y is selected from null, CH₂, CO, and SO₂.

In another refinement, Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, halogen, oxo, CN, NO₂, OR⁸, SR⁸, N(R⁸)R⁹, COR⁸, CO₂R⁸, CON(R⁸)R⁹, SOR⁸, SO₂R⁸, SO₂N(R⁸)R⁹, NR¹⁰COR⁸, NR¹⁰SOR⁸, NR¹⁰SO₂R⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is optionally substituted with one or more substituents independently selected from hydrogen, halogen, oxo, CN, NO₂, OR⁸, NR⁸R⁹, NR¹⁰COR⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, CH₃, CF₃, iPr, cPr, OCH₃, OCF₃, OiPr, OcPr, F, Cl, and Br.

In another refinement, Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from H and F.

In another refinement, R¹ is selected from null, O, NH, CO, CONH, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted aryl, and optionally substituted heteroaryl.

In another refinement, R¹ is selected from null, optionally substituted 3-10 membered carbocyclyl, and optionally substituted 4-10 membered heterocyclyl.

In another refinement, R¹ is selected from null and optionally substituted 4-10 membered heterocyclyl, which contains at least one of O or N.

In another refinement, R¹ is selected from null, optionally substituted piperidinyl, optionally substituted piperazinyl, optionally substituted morpholinyl, optionally substituted pyrrolidinyl, optionally substituted tetrahydrofuranyl, optionally substituted azetidinyl, and optionally substituted oxetanyl.

In another refinement, R² is selected from hydrogen, halogen, optionally substituted C₁-C₈ alkyl, optionally substituted C₂-C₈ alkenyl, optionally substituted C₂-C₈ alkenyl, optionally substituted 3-10 membered carbocyclyl, and optionally substituted 4-10 membered heterocyclyl.

In another refinement, R² is selected from hydrogen, halogen, optionally substituted C₁-C₈ alkyl, optionally substituted 3-10 membered carbocyclyl, and optionally substituted 4-10 membered heterocyclyl.

In another refinement, R² is selected from CH₃, CF₃, iPr, cPr, F, Cl, Br, optionally substituted piperidinyl, optionally substituted optionally substituted piperazinyl, optionally substituted morpholinyl, optionally substituted pyrrolidinyl, optionally substituted tetrahydrofuranyl, optionally substituted azetidinyl, and optionally substituted oxetanyl.

In another refinement, R³, at each occurrence, R¹³ and R¹⁴ are independently selected from hydrogen, CH₃, CF₃, iPr, cPr, tBu, CNCH₂, F, Cl, Br, OH, NH₂, CN, CH₃, and CONH₂.

In another embodiment, the JAK ligand comprises a moiety of FORMULA 2:

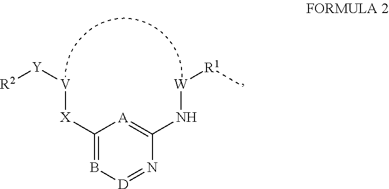

wherein

the linker moiety of the heterobifunctional compound is attached to R¹;

A, B, and D are independently selected from CR³ and N, with the proviso that not all of A, B, and D are N, wherein

R³ is selected from hydrogen, halogen, hydroxyl, amino, cyano, nitro, CONR⁴R⁵, optionally substituted C₁-C₈ alkyl, and optionally substituted 3-10 membered carbocyclyl, wherein

R⁴ and R⁵ are independently selected from hydrogen, optionally substituted C₁-C₈ alkyl, optionally substituted C₂-C₈ alkenyl, optionally substituted C₂-C₈ alkynyl, optionally substituted 3-10 membered carbocyclyl, and optionally substituted 4-10 membered heterocyclyl, or

R⁴ and R⁵ together with the atom or atoms to which they are connected form a 3-20 membered carbocyclyl ring or 4-20 membered heterocyclyl ring;

X and Y are independently selected from null, or a bivalent moiety selected from null, C(R⁶)R⁷, CO, CO₂, CONR⁶, NR⁶, NR⁶CO, NR⁶CO₂, NR⁶C(O)NR⁷, NR⁶SO, NR⁶SO₂, NR⁶SO₂NR⁷, O, OC(O), OCO₂, OCONR⁶, S, SO, SO₂, and SO₂NR⁶, wherein

R⁶ and R⁷ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkylamino, optionally substituted 3-10 membered carbocyclyl, optionally substituted 4-10 membered heterocyclyl, optionally substituted aryl, and optionally substituted heteroaryl, or

R⁶ and R⁷ together with the atom or atoms to which they are connected form a 3-20 membered carbocyclyl ring or 4-20 membered heterocyclyl ring;

V and W are independently selected from null, carbocyclyl, heterocyclyl, aryl, and heteroaryl, which are optionally substituted with one or more substituents independently selected from hydrogen, halogen, oxo, CN, NO₂, OR⁸, SR⁸, N(R⁸)R⁹, OCOR⁸, OCO₂R⁸, OCON(R⁸)R⁹, COR⁸, CO₂R⁸, CON(R⁸)R⁹, SOR⁸, SO₂R⁸, SO₂N(R⁸)R⁹, NR¹⁰CO₂R⁸, NR¹⁰COR⁸, NR¹⁰C(O)N(R⁸)R⁹, NR¹⁰SOR⁸, NR¹⁰SO₂R⁸, NR¹⁰SO₂N(R⁸)R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring;

When neither of V and W is null, V and W together with the substituents to which they are connected optionally form a 10-30 membered macrocyclic ring; When W is null and V is not null, V and R¹ together with the substituents to which they are connected optionally form a 10-30 membered macrocyclic ring; When V is null and W is not null, W and R² together with the substituents to which they are connected optionally form a 10-30 membered macrocyclic ring; or When W and V are null, R¹ and R² together with the substituents to which they are connected optionally form a 10-30 membered macrocyclic ring;

R¹ is connected to the “linker” moiety of the heterobifunctional compound, and is selected from null, R′—R″, R′OR″, R′SR″, R′NR¹¹R″, R′OC(O)R″, R′OC(O)OR″, R′OCONR¹¹R″, R′C(O)R″, R′C(O)OR″, R′CON(R¹¹)R″, R′S(O)R″, R′S(O)₂R″, R′SO₂N(R¹¹)R″, R′NR¹²C(O)OR″, R′NR¹²C(O)R″, R′NR¹²C(O)N(R¹¹)R″, R′NR¹²S(O)R″, R′NR¹²S(O)₂R″, and R′NR¹²S(O)₂N(R¹¹)R″, wherein

R′ and R″ are independently selected from null, optionally substituted C₁-C₈ alkyl, optionally substituted C₂-C₈ alkenyl, optionally substituted C₂-C₈ alkynyl, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused carbocyclyl, optionally substituted C₄-C₁₃ fused heterocyclyl, optionally substituted C₃-C₁₃ bridged carbocyclyl, optionally substituted C₄-C₁₃ bridged heterocyclyl, optionally substituted C₃-C₁₃ spiro carbocyclyl, optionally substituted C₄-C₁₃ spiro heterocyclyl, optionally substituted aryl, and optionally substituted heteroaryl;

R¹¹ and R¹² are independently selected from optionally substituted C₁-C₈ alkyl, optionally substituted C₂-C₈ alkenyl, optionally substituted C₂-C₈ alkynyl, optionally substituted 3-10 membered carbocyclyl, optionally substituted 4-10 membered heterocyclyl, optionally substituted aryl, and optionally substituted heteroaryl;

R′ and R″, R¹¹ and R¹², R′ and R¹¹, R′ and R¹², R″ and R¹¹, R″ and R¹², together with the atom to which they are connected, form a 3-20 membered carbocyclyl or 4-20 membered heterocyclyl ring; and

R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted 3-10 membered carbocyclyl, optionally substituted 4-10 membered heterocyclyl, optionally substituted aryl, and optionally substituted heteroaryl.

In another refinement, V is Ar².

In another refinement, the JAK ligand comprises a moiety of FORMULA 2A:

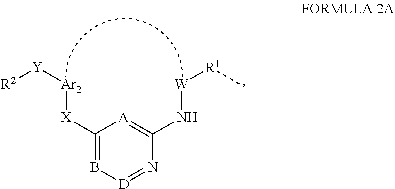

wherein

the linker moiety of the heterobifunctional compound is attached to R¹;

A, B, D, X, Y, W, R¹, and R² are the same as defined in FORMULA 2;

Ar² is selected from null, aryl, and heteroaryl(preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring; and

When neither of W and Ar² is null, W and Ar² together with the substituents to which they are connected optionally form a 10-30 membered macrocyclic ring; When W is null and Ar² is not null, Ar² and R¹ together with the substituents to which they are connected optionally form a 10-30 membered macrocyclic ring; When Ar² is null and W is not null, W and R² together with the substituents to which they are connected optionally form a 10-30 membered macrocyclic ring; or When W and Ar² are null, R¹ and R² together with the substituents to which they are connected optionally form a 10-30 membered macrocyclic ring.

In another refinement, V is Ar²; and W is Ar¹.

In another refinement, the JAK ligand comprises a moiety of FORMULA 2B:

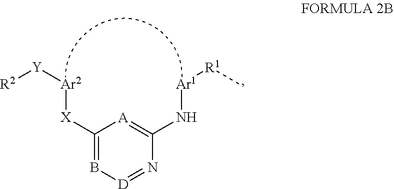

wherein

the linker moiety of the heterobifunctional compound is attached to R¹;

A, B, D, X, Y, R¹, and R² are the same as defined in FORMULA 2; and

Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, halogen, oxo, CN, NO₂, OR⁸, SR⁸, N(R⁸)R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring; and

When neither of Ar¹ and Ar² is null, Ar¹ and Ar² together with the substituents to which they are connected optionally form a 10-30 membered macrocyclic ring; When Ar¹ is null and Ar² is not null, Ar² and R¹ together with the substituents to which they are connected optionally form a 10-30 membered macrocyclic ring; When Ar² is null and Ar¹ is not null, Ar¹ and R² together with the substituents to which they are connected optionally form a 10-30 membered macrocyclic ring; or when Ar¹ and Ar² are null, R¹ and R² together with the substituents to which they are connected optionally form a 10-30 membered macrocyclic ring.

In another refinement, the JAK ligand comprises a moiety of FORMULAE 2C, 2D, 2E or 2F:

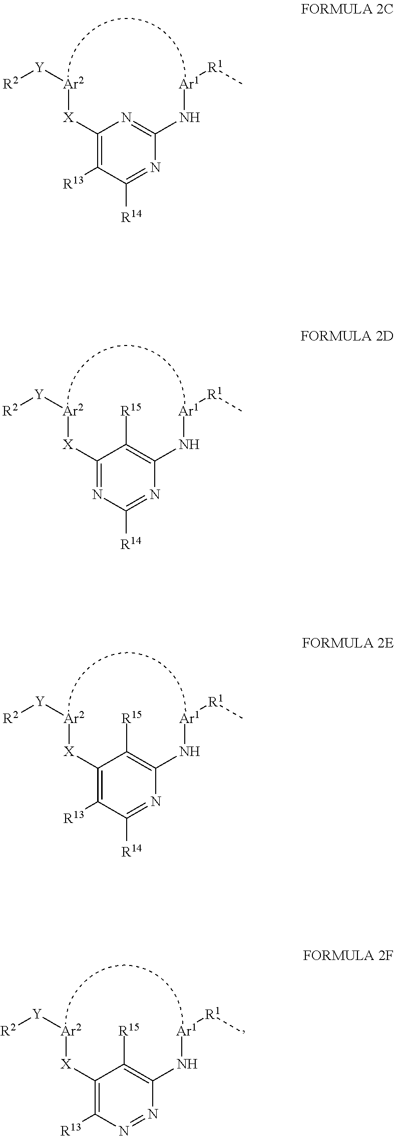

wherein

the linker moiety of the heterobifunctional compound is attached to R¹;

X, Y, Ar¹, Ar², R¹, and R² are the same as defined in FORMULA 2; and

R¹³, R¹⁴ and R¹⁵ are selected from hydrogen, halogen, hydroxyl, amino, cyano, nitro, CONR⁴R⁵, optionally substituted C₁-C₈ alkyl, and optionally substituted 3-10 membered carbocyclyl, wherein

R⁴ and R⁵ are independently selected from hydrogen, optionally substituted C₁-C₈ alkyl, optionally substituted C₂-C₈ alkenyl, optionally substituted C₂-C₈ alkynyl, optionally substituted 3-10 membered carbocyclyl, and optionally substituted 4-10 membered heterocyclyl, or

R⁴ and R⁵ together with the atom or atoms to which they are connected form a 3-20 membered carbocyclyl ring or 4-20 membered heterocyclyl ring.

In another refinement, the JAK ligand comprises a moiety of FORMULAE 2G, 2H, 2I, 2J, 2K, 2L, 2M, 2N, 2O, 2P, 2Q, 2R or 2S:

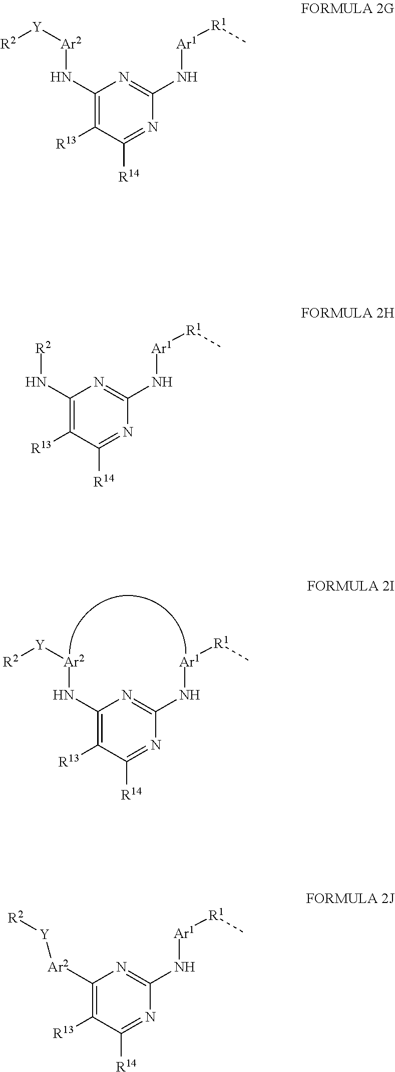

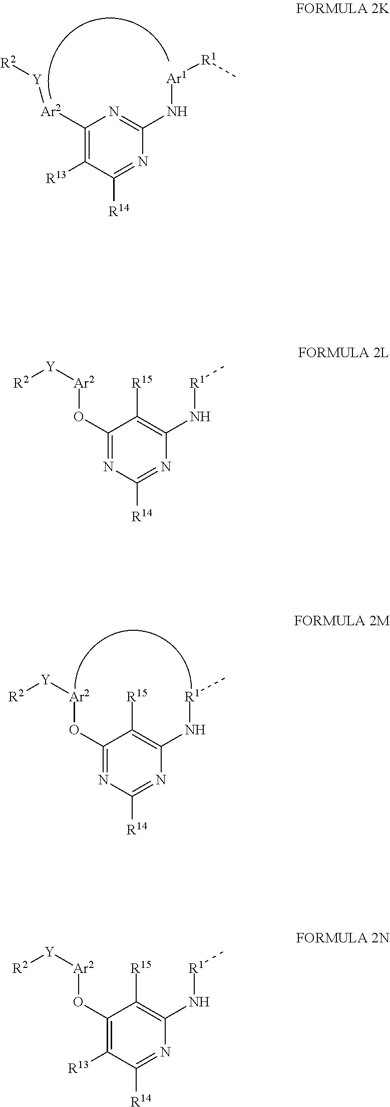

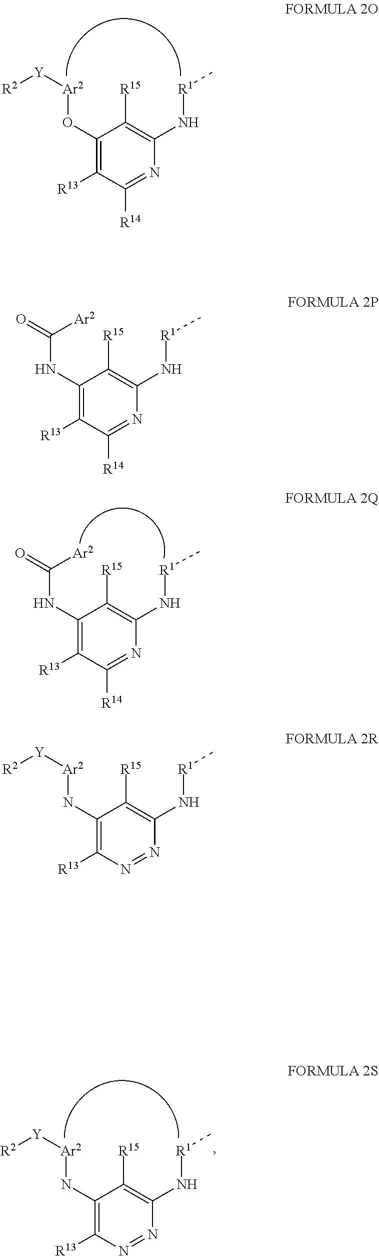

wherein

the linker moiety of the heterobifunctional compound is attached to R¹;

Y, R¹ and R² are the same as defined in FORMULA 2;

Ar¹ and Ar² are the same as defined in FORMULA 2B; and

R¹³, R¹⁴ and R¹⁵ are the same as defined in FORMULAE 2C, 2D, 2E or 2F.

In another refinement, Y is selected from null, CR⁶R⁷, CO, CO₂, CONR⁶, NR⁶CO, NR⁶C(O)NR⁷, O, SO, SO₂, SO₂NR⁶ and NR⁶, wherein

R⁶ and R⁷ are independently selected from hydrogen, optionally substituted C₁-C₈ alkyl, optionally substituted 3-10 membered carbocyclyl, and optionally substituted 3-10 membered heterocyclyl.

In another refinement, Y is selected from null, CH₂, CO, CONH, NR⁶C(O), NR⁶C(O)NR⁷, SO₂ and SO₂NH.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is optionally substituted with one or more substituents independently selected from hydrogen, halogen, oxo, CN, NO₂, OR⁸, SR⁸, NR⁸R⁹, COR⁸, CO₂R⁸, CONR⁸R⁹, SORB, SO₂R⁸, SO₂NR⁸R⁹, NR¹⁰COR⁸, NR¹⁰SOR⁸, NR¹⁰SO₂R⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, halogen, oxo, CN, NO₂, OR⁸, NR⁸R⁹, NR¹⁰COR⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, aryl, and heteroaryl (preferably selected from null, monocyclic aryl, monocyclic heteroaryl, bicyclic aryl, bicyclic heteroaryl, tricyclic aryl, and tricyclic heteroaryl), each of which is optionally substituted with one or more substituents independently selected from hydrogen, CH₃, CF₃, iPr, cPr, OCH₃, OCF₃, OiPr, OcPr, F, Cl, and Br.

In another refinement, Ar¹ and Ar² are independently selected from null, aryl, and heteroaryl (preferably selected from null monocyclic aryl, monocyclic heteroaryl, bicyclic aryl, bicyclic heteroaryl, tricyclic aryl, and tricyclic heteroaryl), each of which is optionally substituted with one or more substituents independently selected from H and F.

In another refinement, R¹ is selected from null, O, NH, CO, CONH, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted aryl, and optionally substituted heteroaryl.

In another refinement, R¹ is selected from null, O, NH, optionally substituted 3-10 membered carbocyclyl, and optionally substituted 4-10 membered heterocyclyl.

In another refinement, R¹ is selected from null, O, NH, and optionally substituted 4-10 membered heterocyclyl, which contains at least one of O or N.

In another refinement, R¹ is selected from null, O, NH, optionally substituted piperidinyl, optionally substituted piperazinyl, optionally substituted morpholinyl, optionally substituted pyrrolidinyl, optionally substituted tetrahydrofuranyl, optionally substituted azetidinyl, and optionally substituted oxetanyl.

In another refinement, R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted C₁-C₈ alkoxy, optionally substituted 3-10 membered carbocyclyl, optionally substituted 4-10 membered heterocyclyl, optionally substituted aryl, and optionally substituted heteroaryl.

In another refinement, R² is selected from hydrogen, halogen, optionally substituted C₁-C₈ alkyl, optionally substituted 3-10 membered carbocyclyl, optionally substituted 4-10 membered heterocyclyl, optionally substituted aryl, and optionally substituted heteroaryl.

In another refinement, R² is selected from hydrogen, CH₃, CF₃, iPr, cPr, tBu, CNCH₂, F, Cl, Br, optionally substituted piperidinyl, optionally substituted optionally substituted piperazinyl, optionally substituted morpholinyl, optionally substituted pyrrolidinyl, optionally substituted tetrahydrofuranyl, optionally substituted azetidinyl, and optionally substituted oxetanyl, optionally substituted phenyl, optionally substituted triazolyl, optionally substituted pyridinyl, optionally substituted pyrimidinyl, optionally substituted pyrazinyl, optionally substituted triazinyl, optionally substituted fruranyl, optionally substituted oxazolyl, optionally substituted pyrrolyl, optionally substituted imidazolyl, optionally substituted triazolyl, optionally substituted oxadiazolyl, optionally substituted thiophenyl, optionally substituted thiazolyl, and optionally substituted thiadiazolyl.

In another refinement, R¹³, R¹⁴ and R¹⁵ are independently selected from H, CH₃, CF₃, iPr, cPr, tBu, CNCH₂, F, Cl, Br, OH, NH₂, CN, CH₃, and CONH₂.

In another embodiment, the JAK ligand is derived from any of the following:

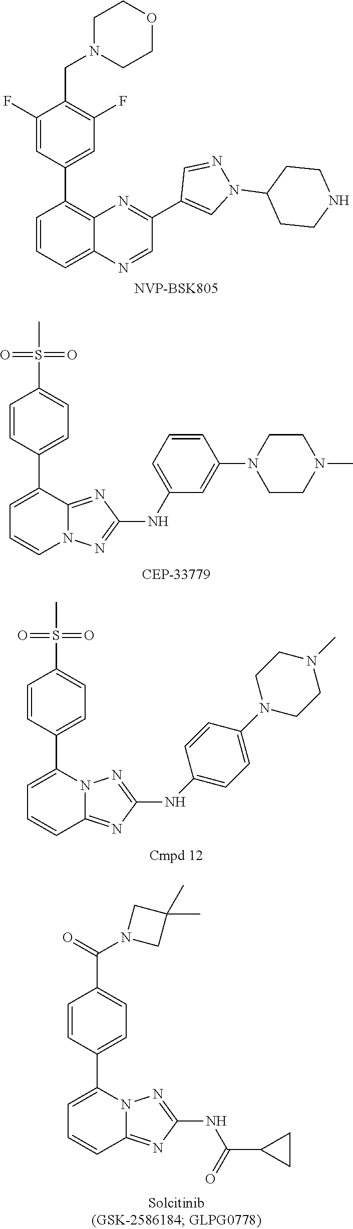

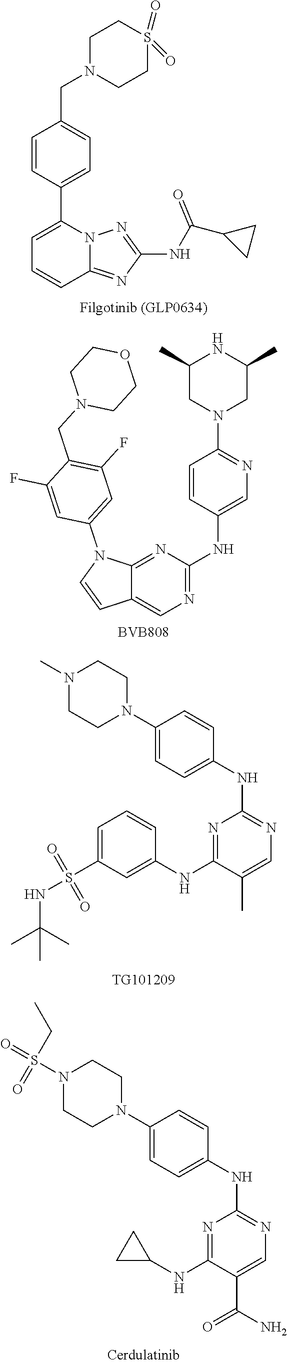

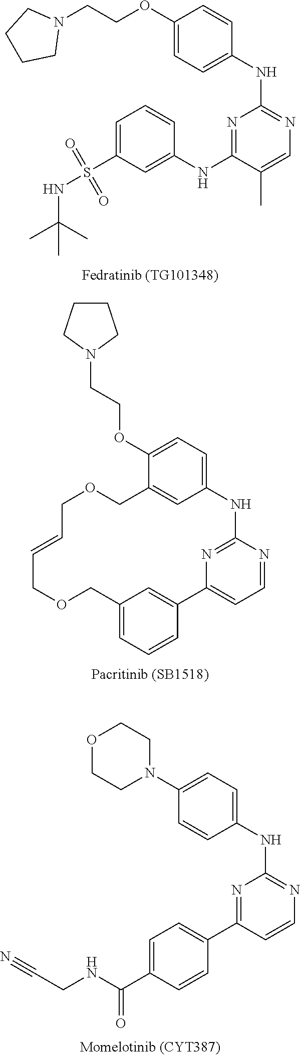 [SMILES: Cc1cnc(Nc2ccc(OCCN3CCCC3)cc2)nc1Nc1cccc(S(=O)(=O)NC(C)(C)C)c1 ; C1=C/COCc2cc(ccc2OCCN2CCCC2)Nc2nccc(n2)-c2cccc(c2)COC/1 ; N#CCNC(=O)c1ccc(-c2ccnc(Nc3ccc(N4CCOCC4)cc3)n2)cc1]

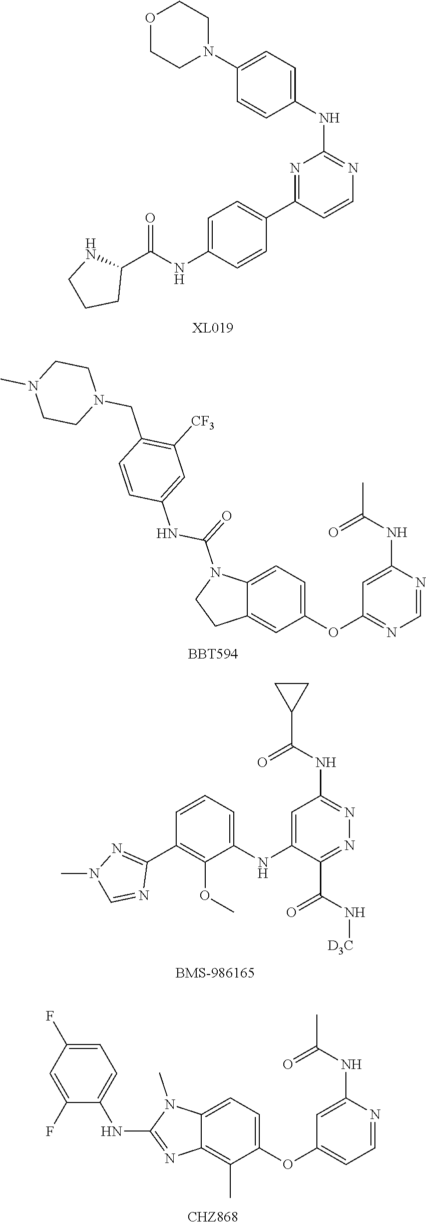

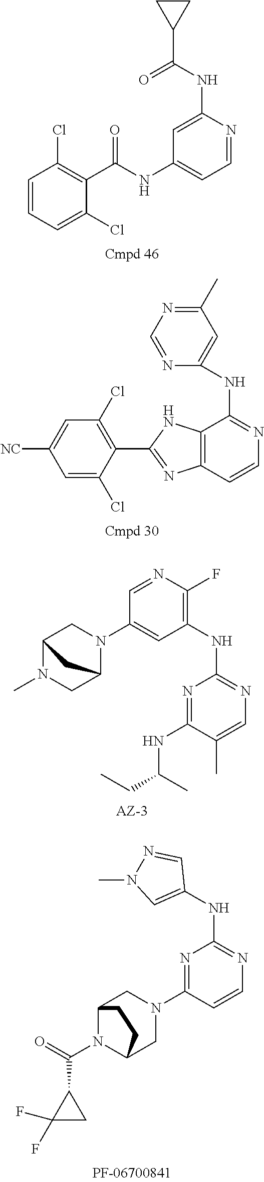

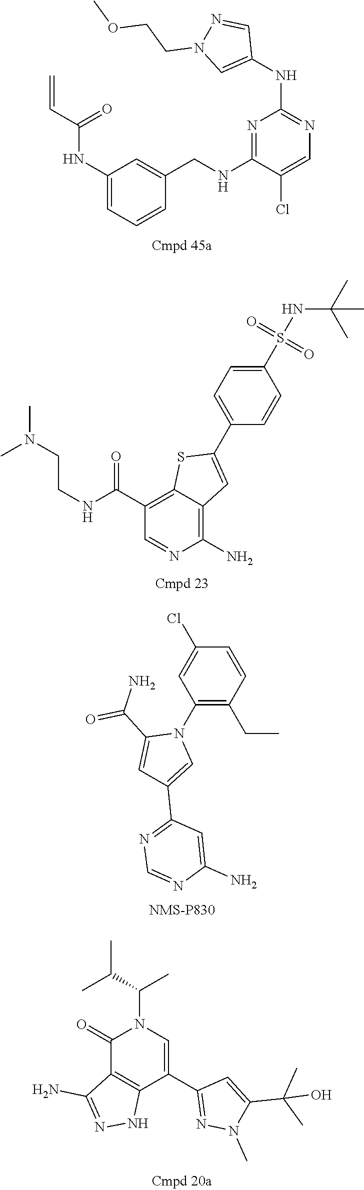 [SMILES: C=CC(=O)Nc1cccc(CNc2nc(Nc3cnn(CCOC)c3)ncc2Cl)c1 ; CN(C)CCNC(=O)c1cnc(N)c2cc(-c3ccc(S(=O)(=O)NC(C)(C)C)cc3)sc12 ; CCc1ccc(Cl)cc1-n1cc(-c2cc(N)ncn2)cc1C(N)=O ; CC(C)[C@H](C)n1cc(-c2cc(C(C)(C)O)n(C)n2)c2[nH]nc(N)c2c1=O]

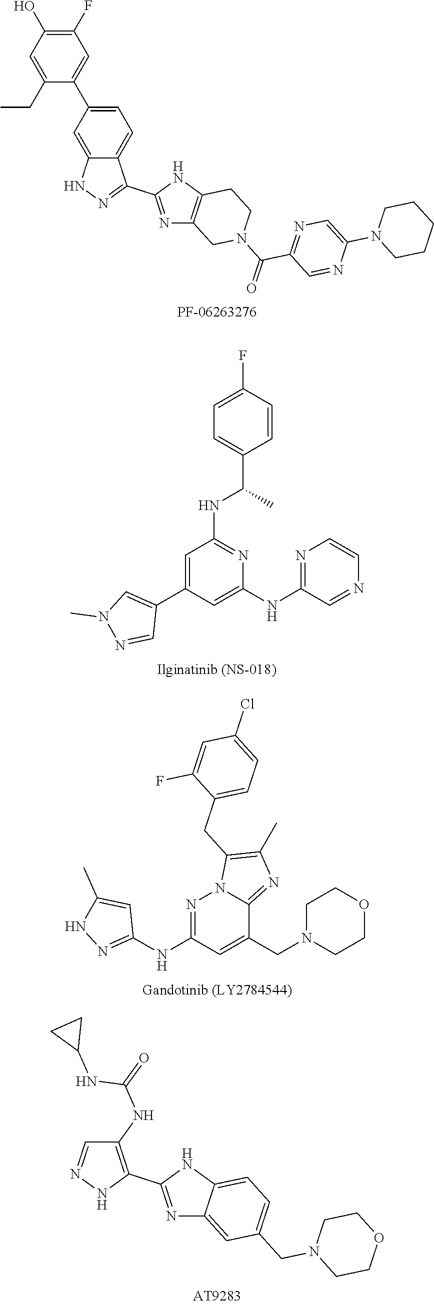 [SMILES: CCc1cc(O)c(F)cc1-c1ccc2c(-c3nc4c([nH]3)CCN(C(=O)c3cnc(N5CCCCC5)cn3)C4)n[nH]c2c1 ; C[C@H](Nc1cc(-c2cnn(C)c2)cc(Nc2cnccn2)n1)c1ccc(F)cc1 ; Cc1cc(Nc2cc(CN3CCOCC3)c3nc(C)c(Cc4ccc(Cl)cc4F)n3n2)n[nH]1 ; O=C(Nc1cn[nH]c1-c1nc2cc(CN3CCOCC3)ccc2[nH]1)NC1CC1]

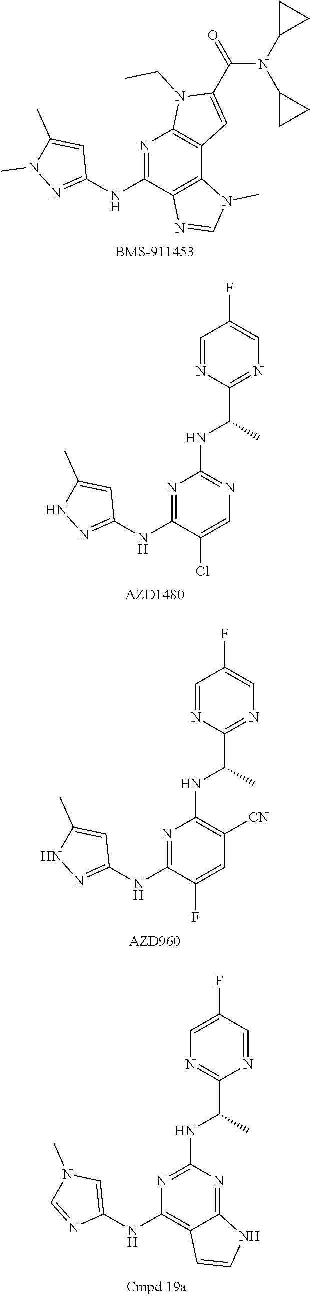 [SMILES: CCn1c(C(=O)N(C2CC2)C2CC2)cc2c3c(ncn3C)c(Nc3cc(C)n(C)n3)nc21 ; Cc1cc(Nc2nc(N[C@@H](C)c3ncc(F)cn3)ncc2Cl)n[nH]1 ; Cc1cc(Nc2nc(N[C@@H](C)c3ncc(F)cn3)c(C#N)cc2F)n[nH]1 ; C[C@H](Nc1nc(Nc2cn(C)cn2)c2cc[nH]c2n1)c1ncc(F)cn1]

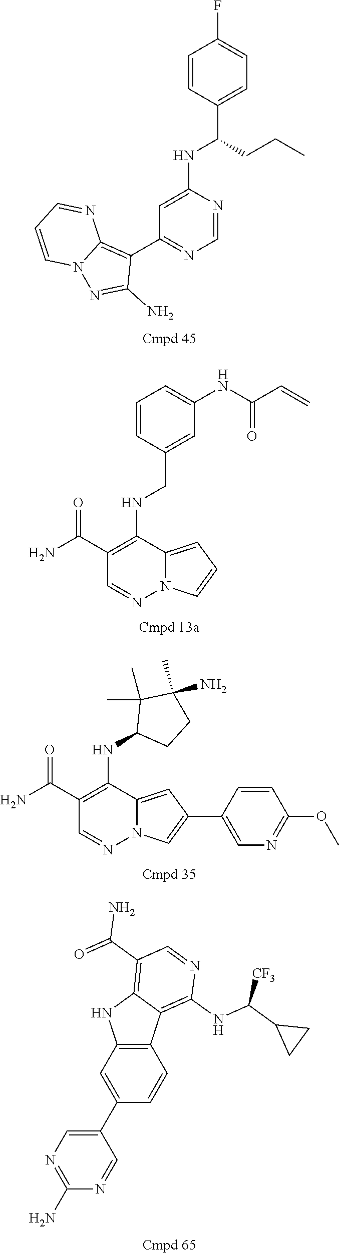 [SMILES: CCC[C@H](Nc1cc(-c2c(N)nn3cccnc23)ncn1)c1ccc(F)cc1 ; C=CC(=O)Nc1cccc(CNc2c(C(N)=O)cnn3cccc23)c1 ; COc1ccc(-c2cc3c(N[C@@H]4CC[C@](C)(N)C4(C)C)c(C(N)=O)cnn3c2)cn1 ; NC(=O)c1cnc(N[C@H](C2CC2)C(F)(F)F)c2c1[nH]c1cc(-c3cnc(N)nc3)ccc12]

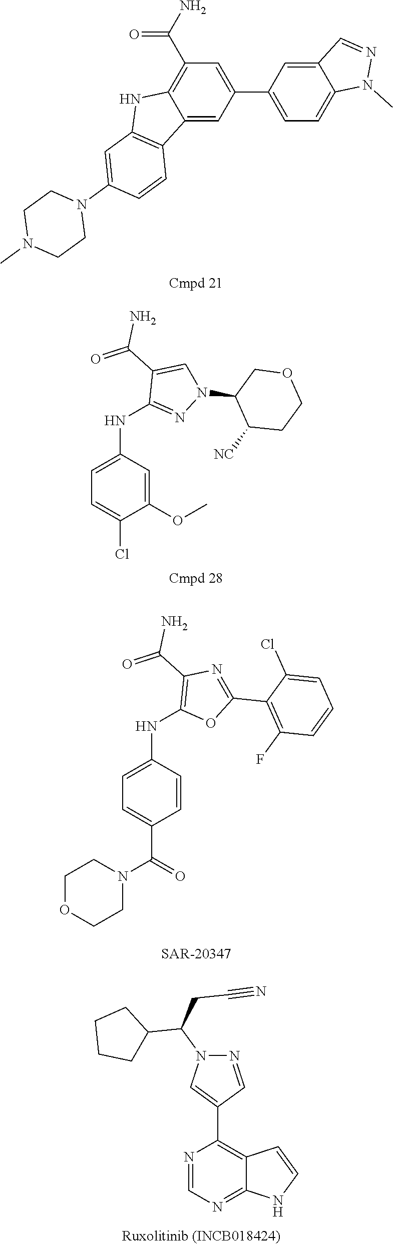

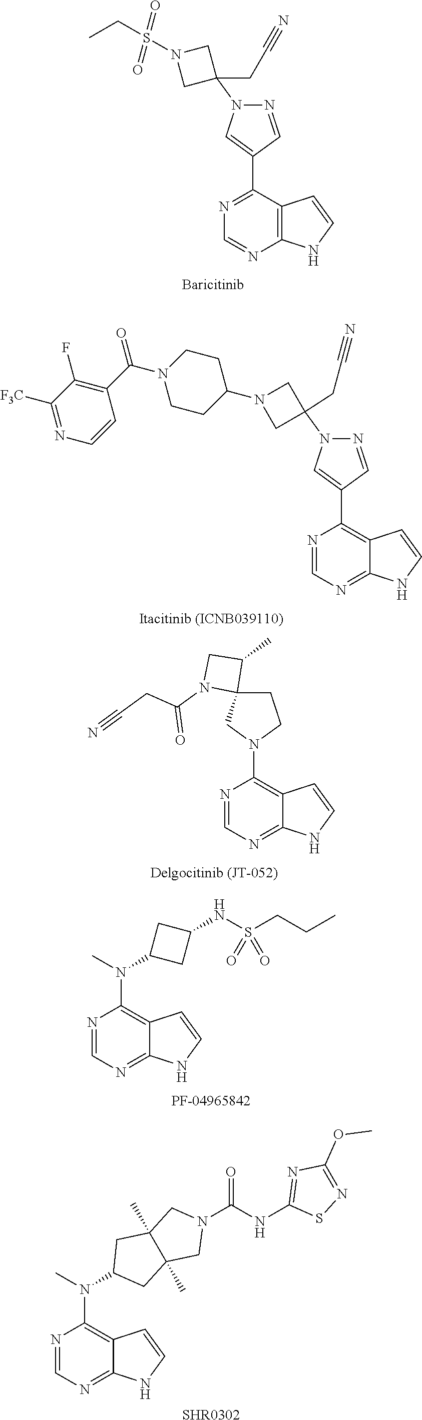

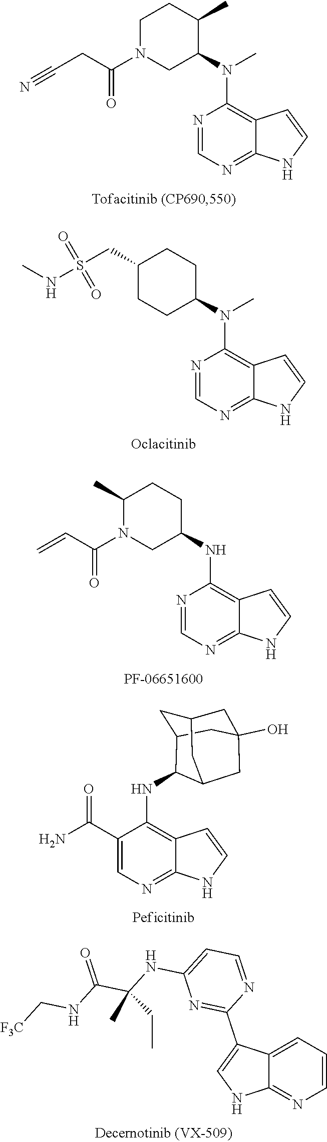

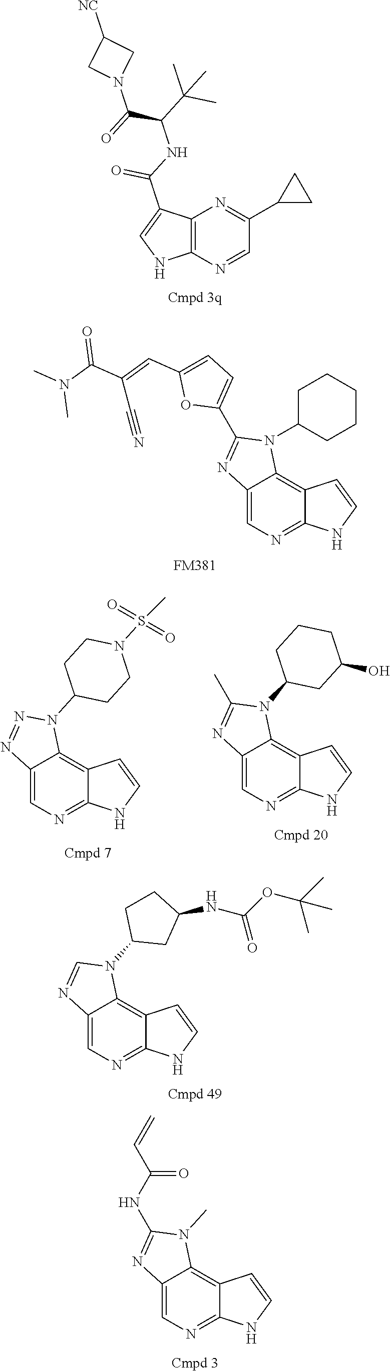

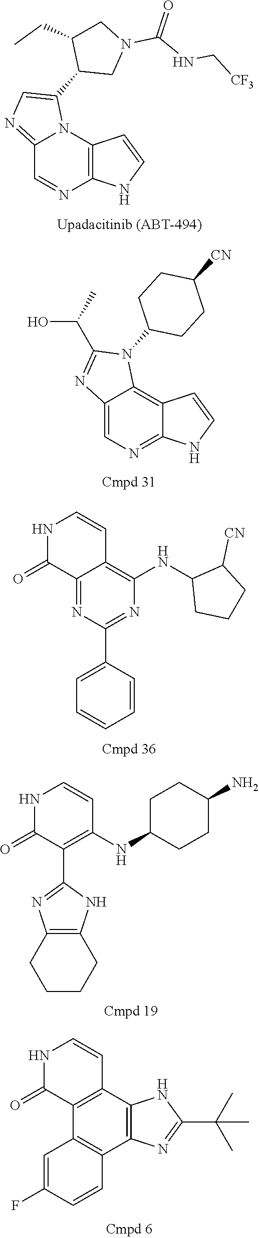

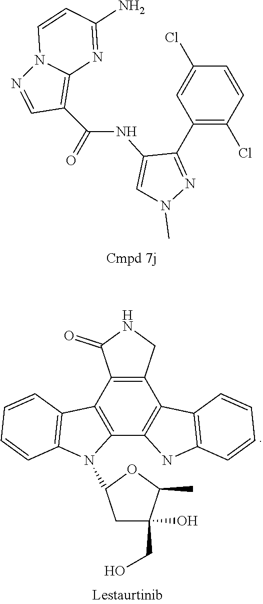

In another embodiment, the JAK ligand is derived from any of the following: NVP-BSK805, Cmpd 12, and TG101209 (preferably, NVP-BSK805 and Cmpd 12).

In another embodiment, the JAK ligand is derived from the following JAK inhibitors: NDI-031301, NDI-31232, VR588, R333 and R348.

In another embodiment, the JAK ligand is selected from the group consisting of:

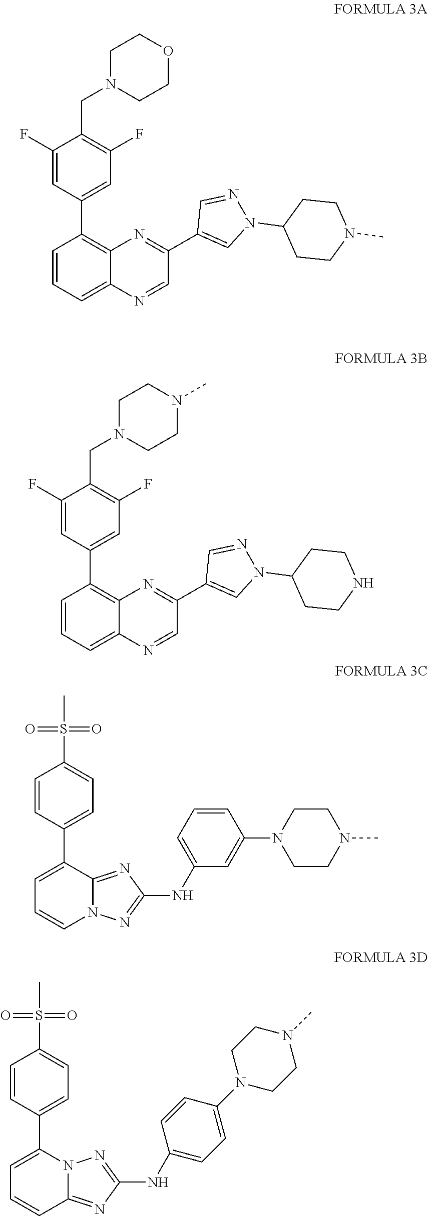

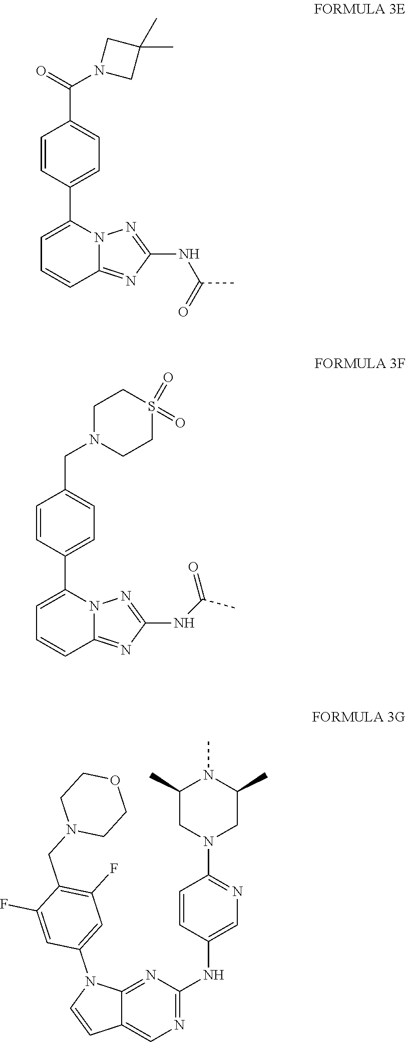

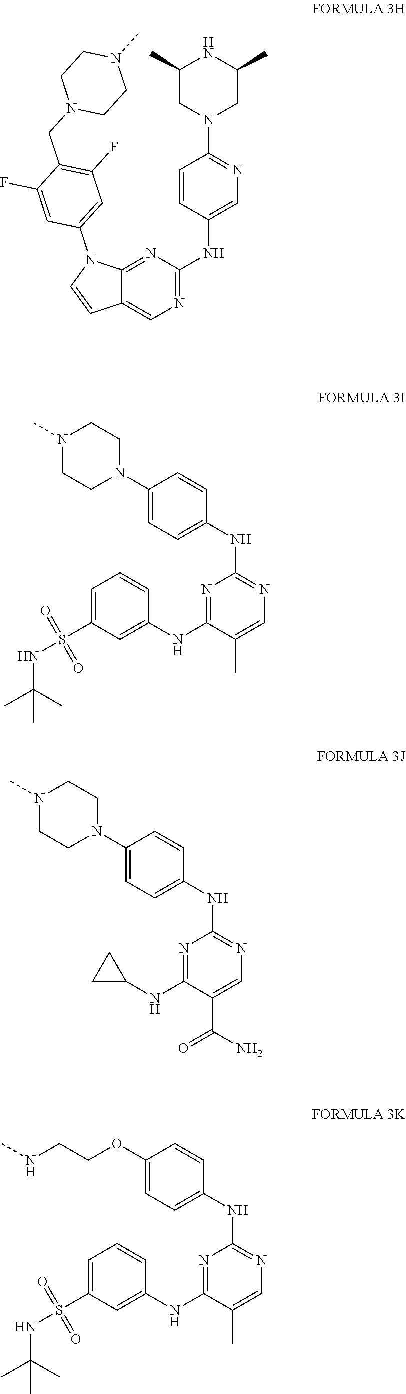

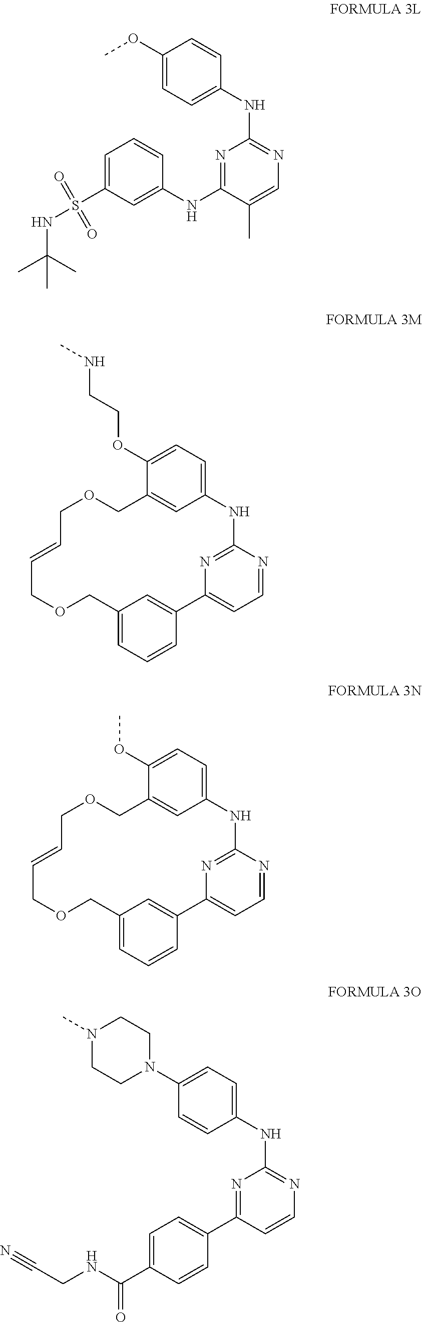

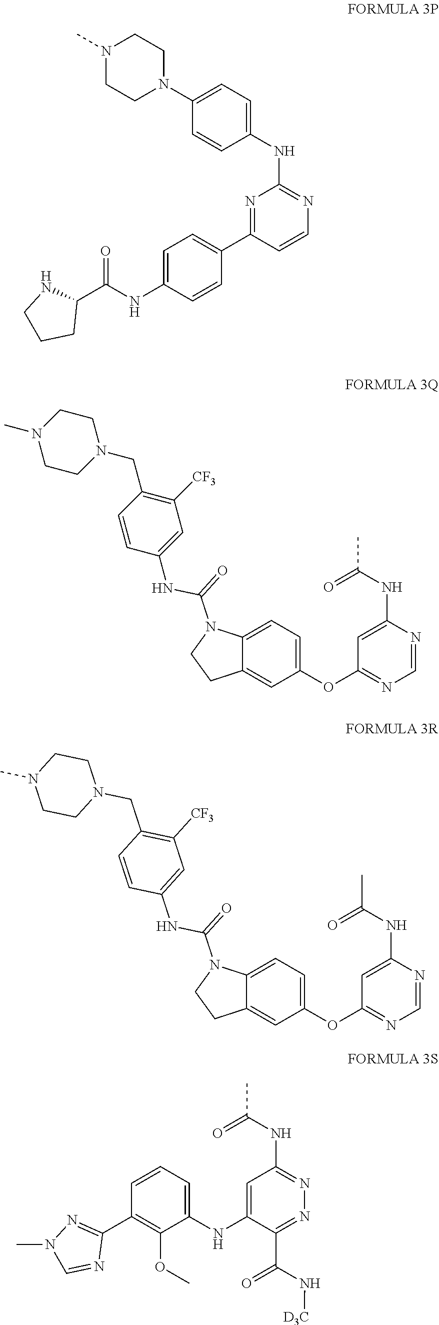

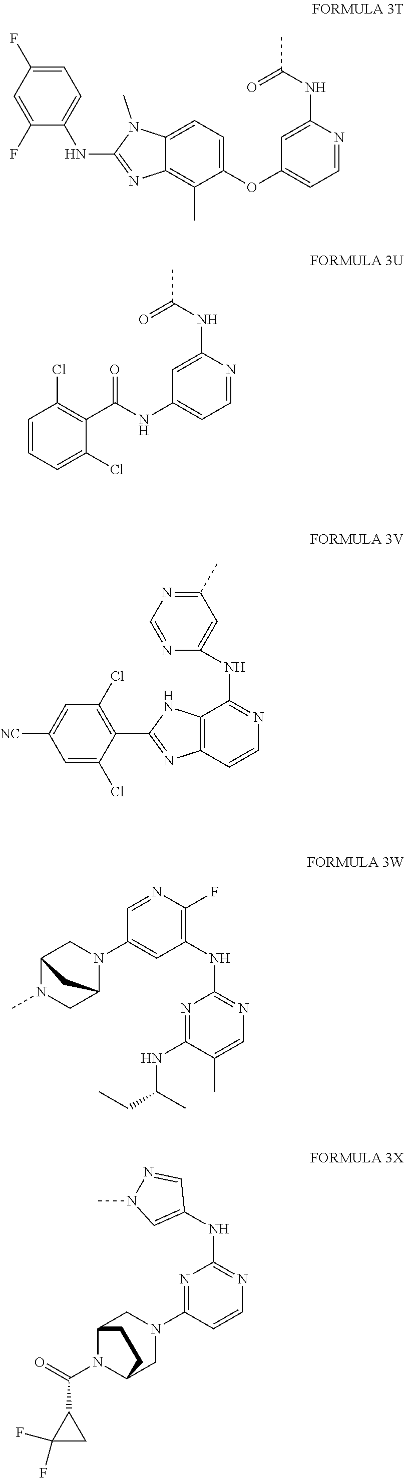

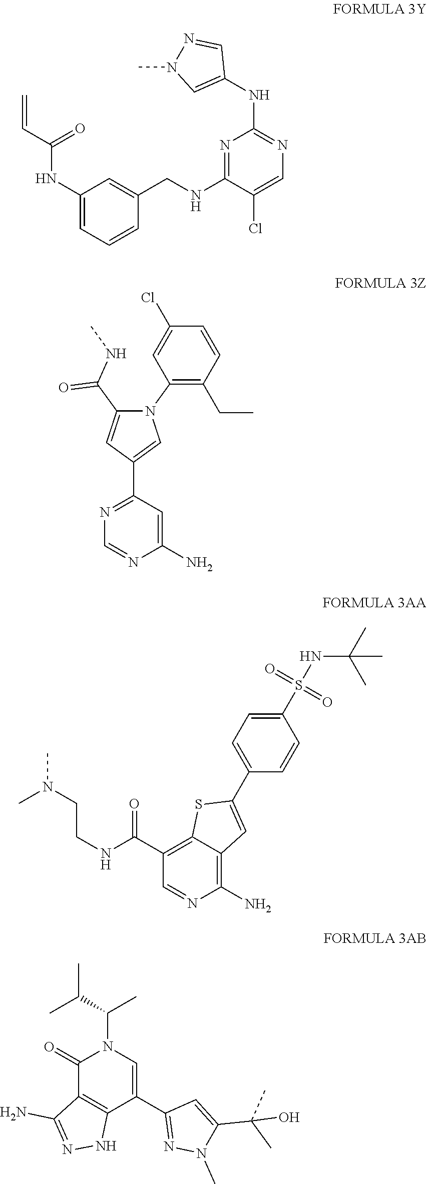

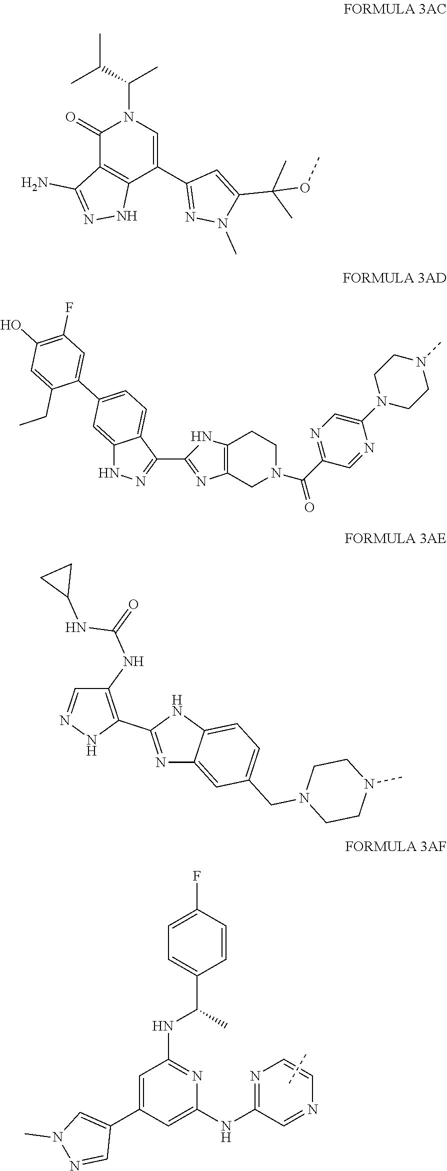

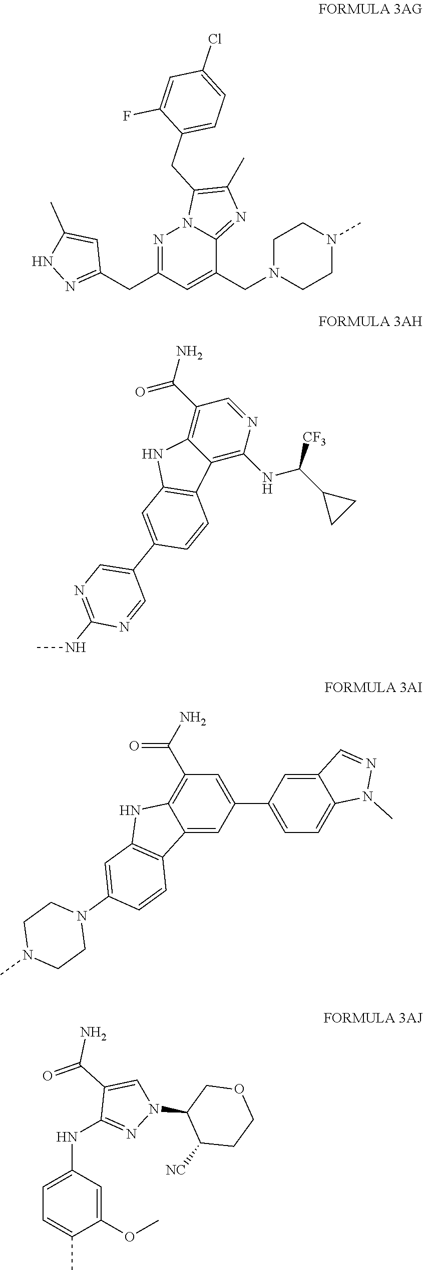

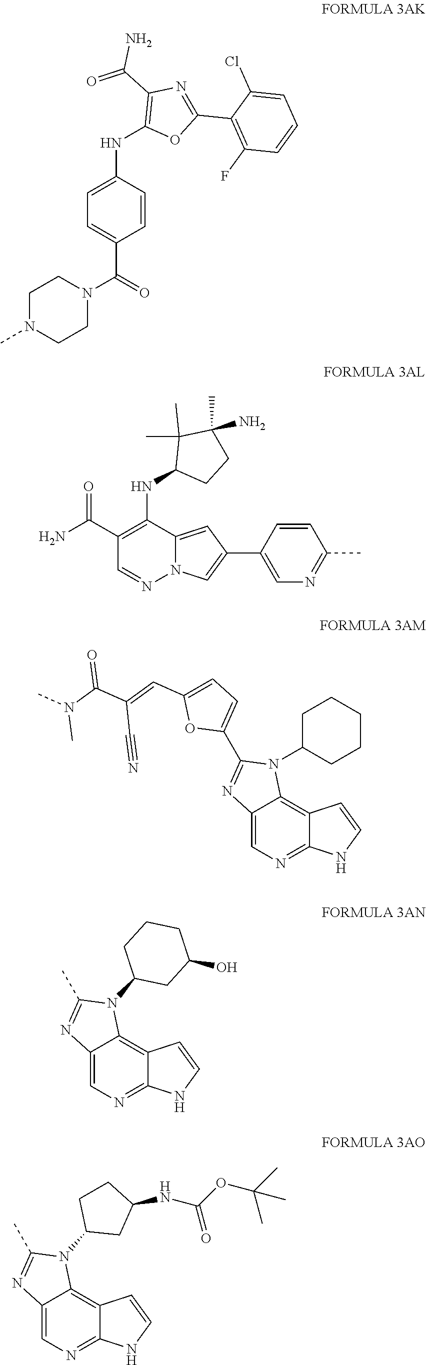

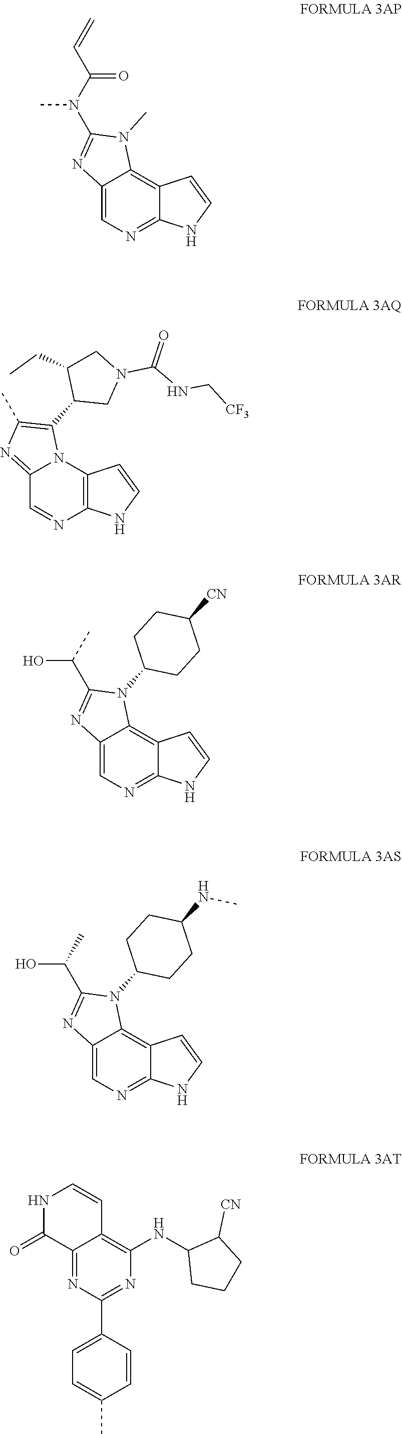

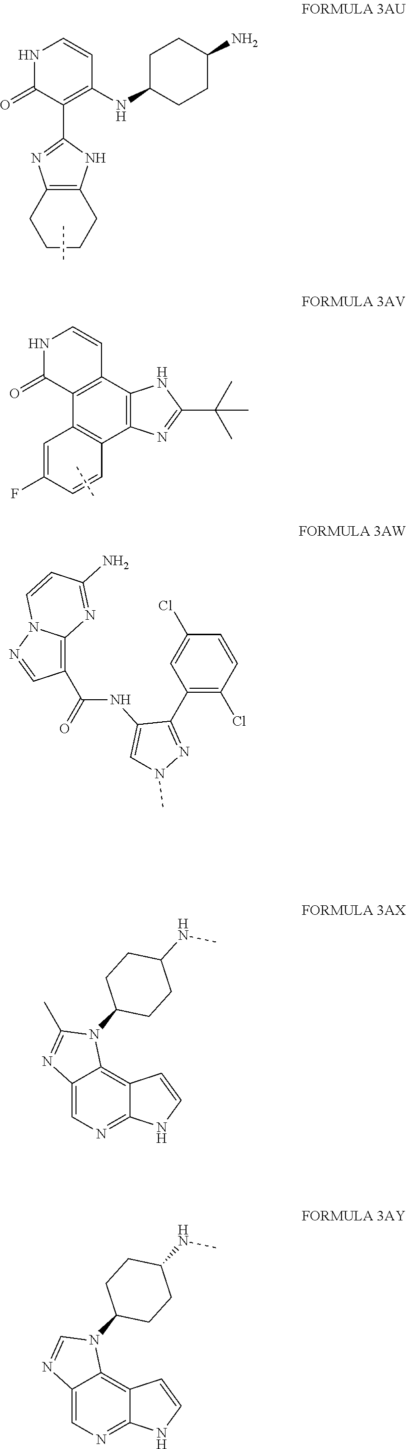

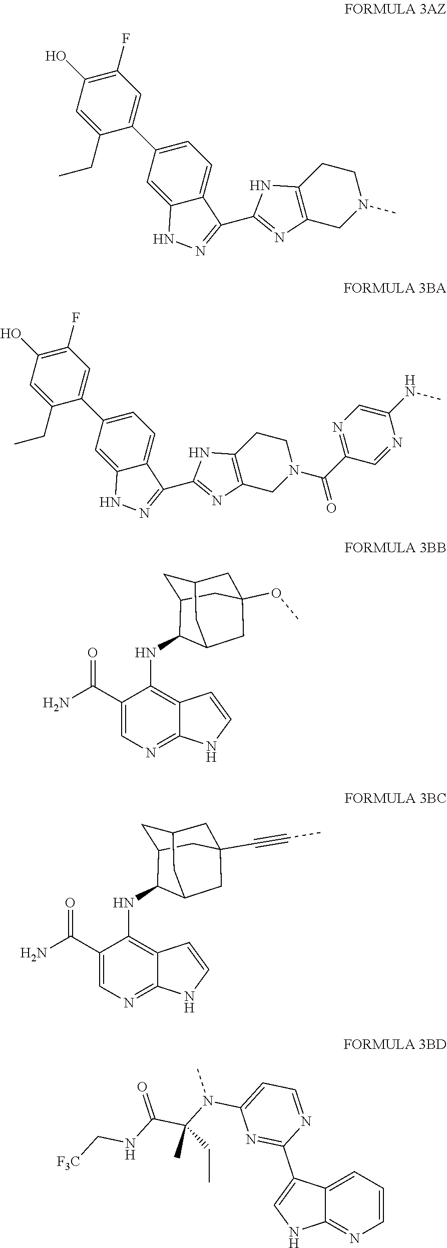

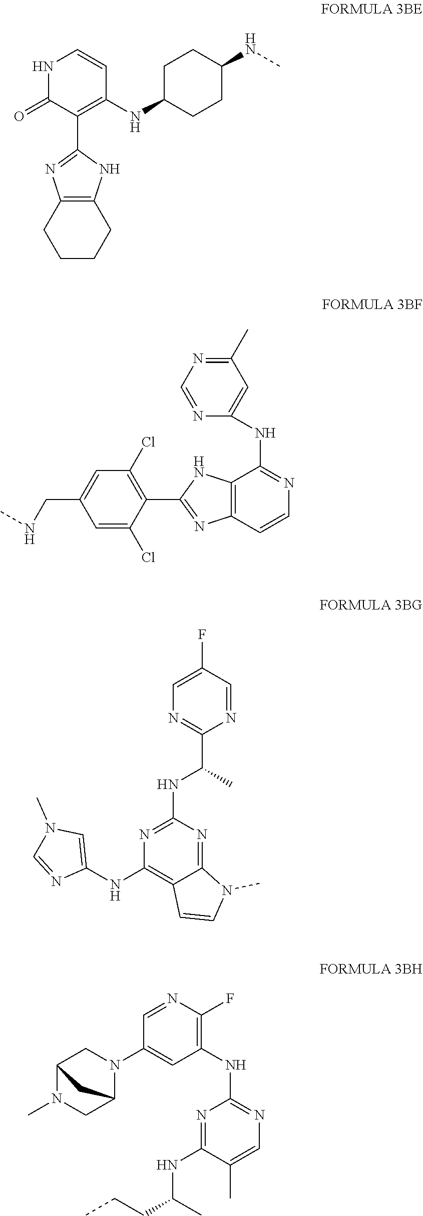

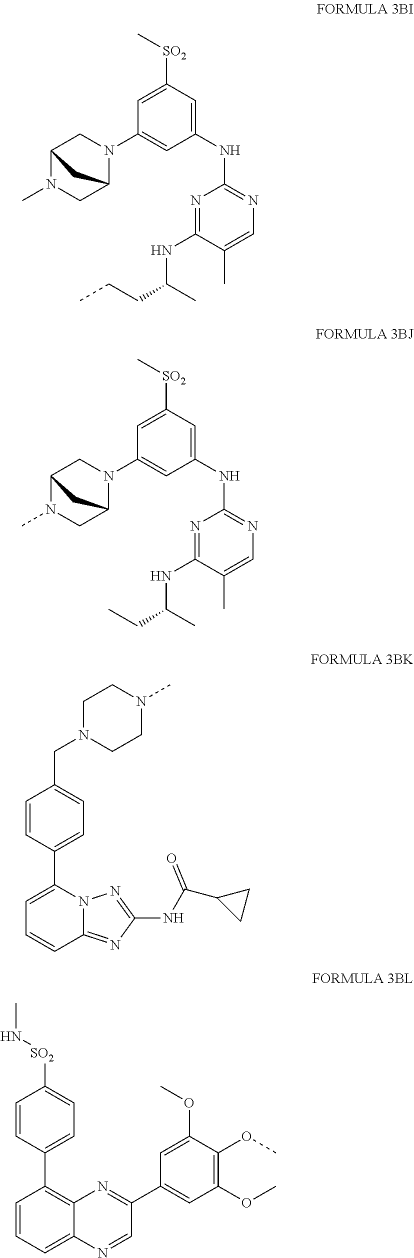

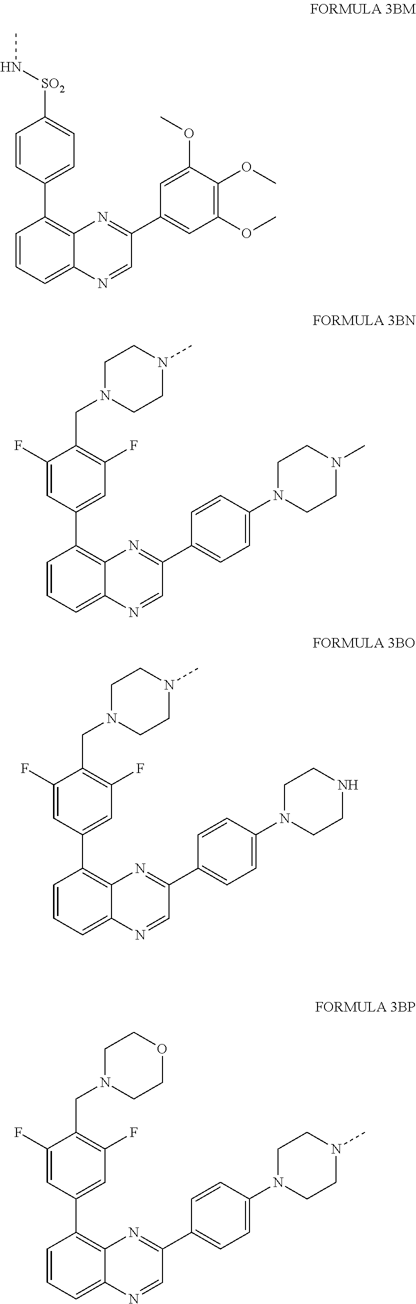

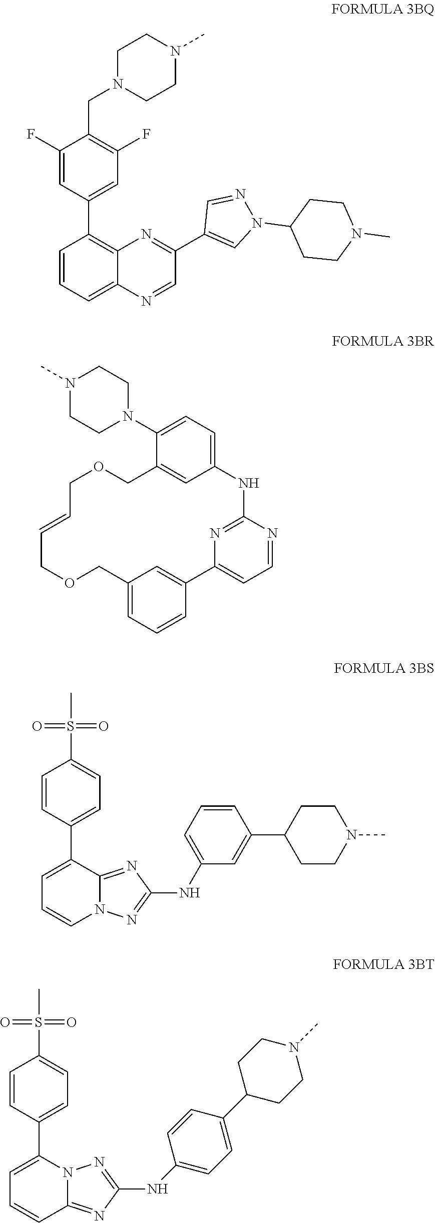

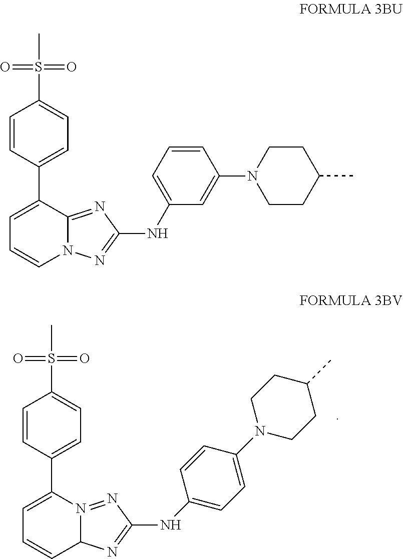

In another embodiment, the JAK ligand is selected from FORMULA 3A, FORMULA 3C, FORMULA 3D), and FORMULA 31; preferably, selected from FORMULA 3A, FORMULA 3C, and FORMULA 3D.

In some embodiments, the degradation tag is a moiety selected from the group consisting of FORMULAE 5A, 5B, 5C, and 5D:

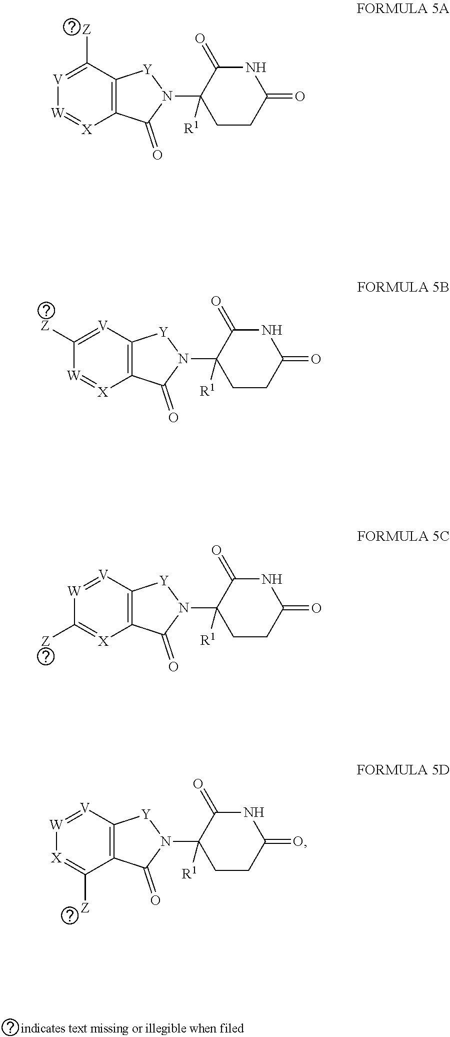

wherein

V, W, and X are independently selected from CR² and N;

Y is selected from —CO—, —CR³R⁴—, —N═CR³—, and —N═N—; preferably, Y is selected from —CO—, —CH₂—, and —N═N—;

Z is selected from null, CO, CR⁵R⁶, NR⁵, O, C≡C, optionally substituted C₁-C₁₀ alkylene, optionally substituted C₂-C₁₀ alkenyl, and optionally substituted C₂-C₁₀ alkynyl, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, NH, O, and C≡C;

R¹, R², R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl, or

R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl, or

R⁵ and R⁶ together with the atom to which they are connected form a 3-6 membered carbocyclyl, or 4-6 membered heterocyclyl.

In another embodiment, R¹, R², R³, R⁴, R⁵ and R⁶ are hydrogen.

In another embodiment, the degradation tag is a moiety selected from the group consisting of FORMULAE 5E, 5F, 5G, 5H, and 5I:

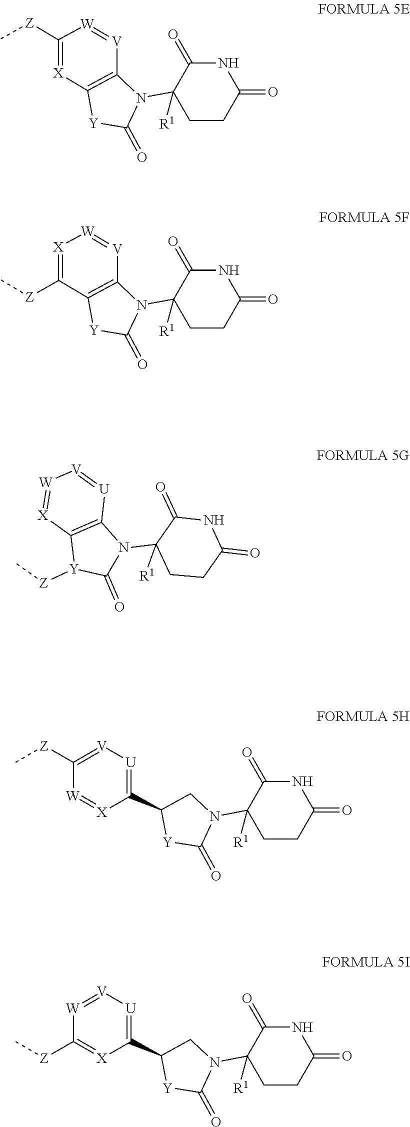

wherein

U, V, W, and X are independently selected from CR² and N;

Y is selected from —N—, —CR³═, —CR³R⁴—, —NR³— and —O—; preferably, Y is selected from —N—, —CH₂—, —NH—, —N(CH₃)— and —O—;

Z is selected from null, CO, CR⁵R⁶, NR⁵, O, optionally substituted C₁-C₁₀ alkylene, optionally substituted C₂-C₁₀ alkenylene, optionally substituted C₂-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and 0;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In another embodiment, R¹, R², R³, R⁴, R⁵ and R⁶ are hydrogen.

In another embodiment, when the degradation tag is a moiety of FORMULAE 5G, Y is selected from —N— and —CR³═; preferably, Y is N.

In another embodiment, when the degradation tag is a moiety of FORMULAE 5E, 5F, 5H, or 5I, Y is selected from —CR³R⁴—, —NR³—, and —O—.; preferably, Y is selected from CH₂, NH, N(CH₃) and 0.

In one embodiment, the degradation tag is a moiety selected from the group consisting of FORMULAE 5J, 5K, 5L, 5M, 5N, 5O, 5P, and 5Q:

wherein

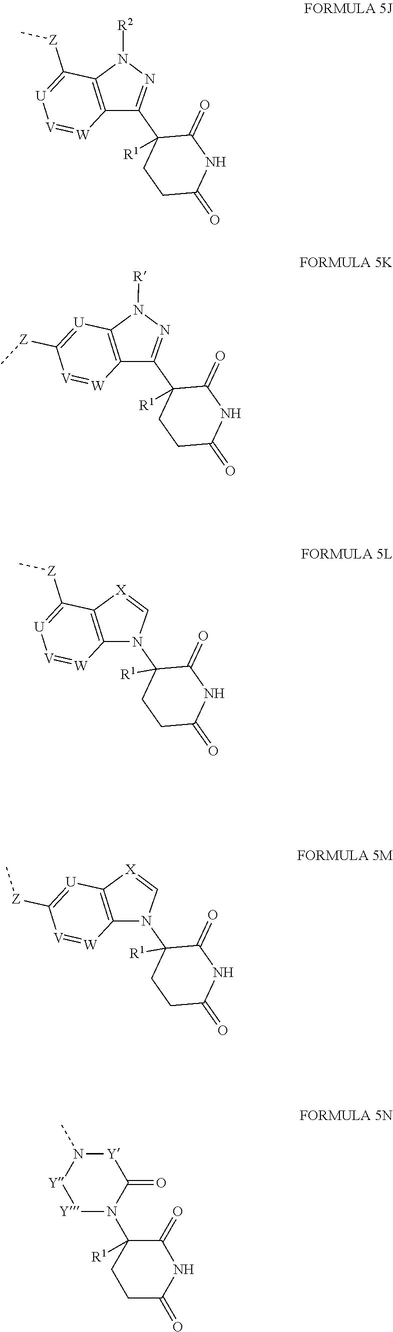

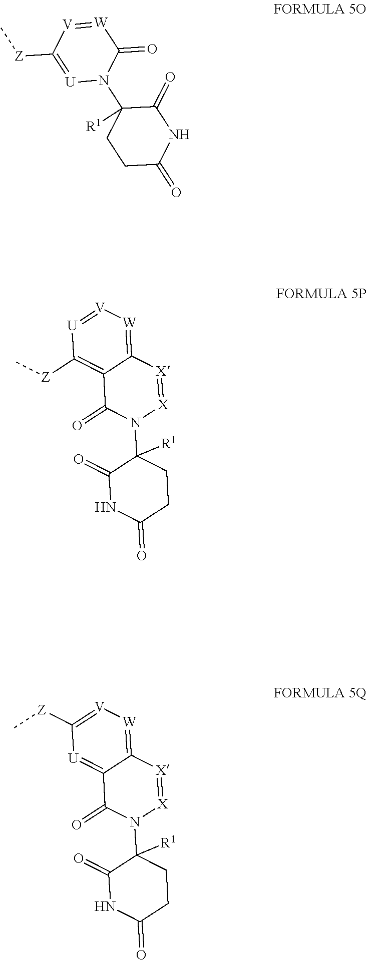

X′ are independently selected from CR² and N;

Y′, Y″, and Y′″ are independently selected from CR³R⁴.

U, V, W, Y, X, Z, R¹, R², R³ and R⁴ are defined as in FORMULAE 5E, 5F, 5G, 5H, or 5I;

R′ is selected from hydrogen, optionally substituted C1-C6 alkyl, optionally substituted 3 to 6 membered carbocyclyl, and optionally substituted 4 to 6 membered heterocyclyl.

In one embodiment, the degradation tag is a moiety of FORMULA 6A:

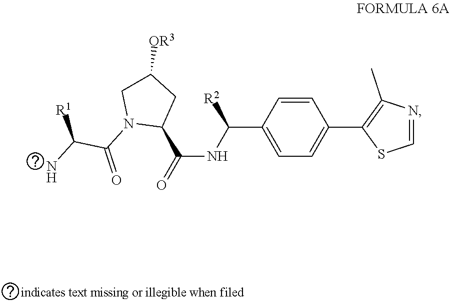

wherein

R¹ and R² are independently selected from hydrogen, hydroxyl, amino, cyano, nitro, optionally substituted C₁-C₈ alkyl, optionally substituted C₂-C₈ alkenyl, and optionally substituted C₂-C₈ alkynyl, optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl; and

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In another embodiment, the degradation tag is a moiety of FORMULAE 6B, 6C, and 6D:

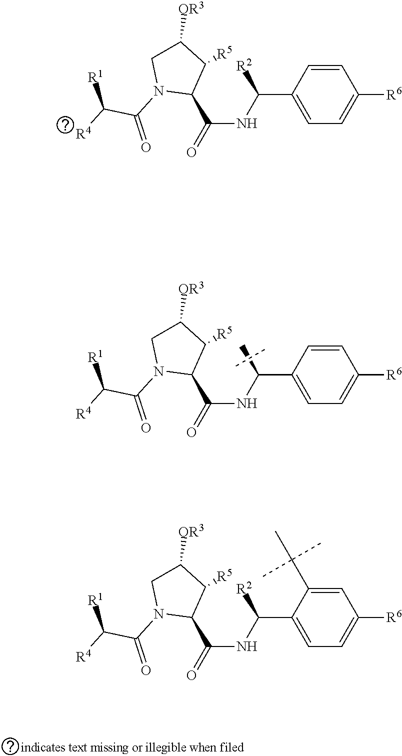

wherein

R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl;

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂, and

R⁴ is selected from NR⁷R⁸

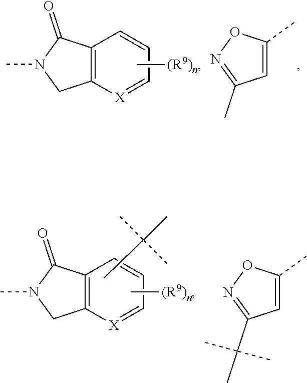

optionally substituted C₁-C₈alkoxy, optionally substituted 3-10 membered carbocyclyl, optionally substituted 4-10 membered heterocyclyl, optionally substituted aryl, and optionally substituted heteraryl, in which

R⁷ is selected from hydrogen, optionally substituted C₁-C₈alkyl, optionally substituted C₁-C₈cycloalkyl, optionally substituted C₁-C₈alkyl-CO, optionally substituted C₁-C₈cycloalkyl-CO, optionally substituted C₁-C₈cycloalkyl-C₁-C₈alkyl-CO, optionally substituted 4-10 membered heterocyclyl-CO, optionally substituted 4-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;

R⁸ is selected from hydrogen, optionally substituted C₁-C₈alkyl, and optionally substituted C₁-C₈cycloalkyl;

R⁹, at each occurrence, is independently selected from hydrogen, halogen, cyano, optionally substituted C₁-C₈alkyl, optionally substituted C₁-C₈cycloalkyl, optionally substituted C₁-C₈heterocycloalkyl, optionally substituted C₁-C₈alkoxy, optionally substituted C₁-C₈cycloalkoxy, halo substituted C₁-C₈alkyl, halo substituted C₁-C₈cycloalkyl, halo substituted C₁-C₈alkoxl, halo substituted C₁-C₈cycloalkoxy, and halo substituted C₁-C₈heterocycloalkyl;

X is selected from CH and N; and

n is 0, 1, 2, 3, or 4;

R⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl, preferably, halogen, cyano, optionally substituted imidazole, optionally substituted pyrazole, optionally substituted oxadiazole, optionally substituted triazole, 4-methylthiazol-5-yl, or oxazol-5-yl group.

In another embodiment, the degradation tag is a moiety of FORMULA 7A:

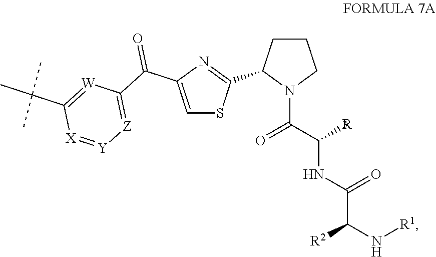

wherein

V, W, X, and Z are independently selected from CR⁴ and N; and

R¹, R², R³, and R⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈alkoxy, optionally substituted C₁-C₈alkylamino, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ hydroxyalkyl, optionally substituted 3-10 membered carbocyclyl, and optionally substituted 4-10 membered heterocyclyl.

In another embodiment, the degradation tag is a moiety of FORMULA 7B:

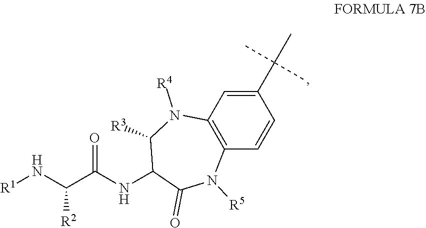

wherein

R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;

R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl, wherein

R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl, or

R⁶ and R⁷ together with the atom to which they are connected form a 4-8 membered cycloalkyl or heterocyclyl ring.

In another embodiment, the degradation tag is a moiety of FORMULA 5A, 5B, 5C, 5D, 5E, 5F, 5G, 5H, and 5I.

In one embodiment, the degradation tag is a moiety selected from the group consisting of FORMULA 5B, FORMULA 5C, FORMULA 5E, and FORMULA 5F.

In one embodiment, the degradation tag is derived from any of the following:

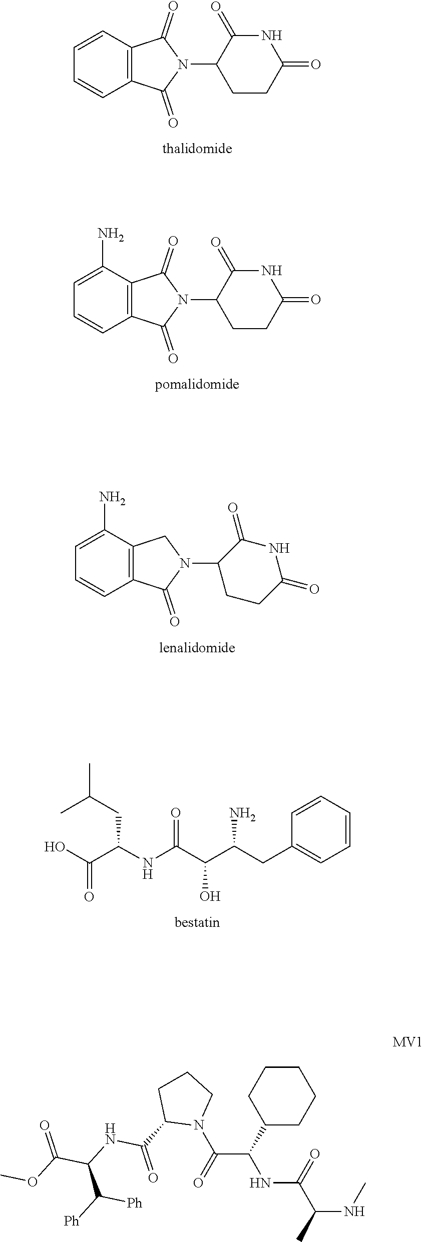

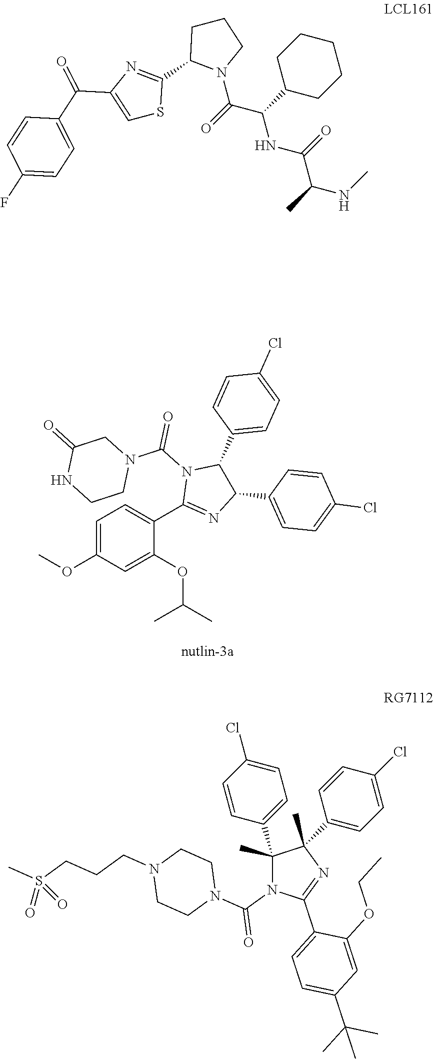

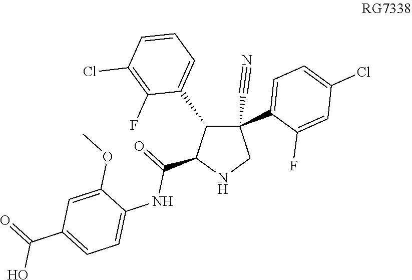 [SMILES: COc1cc(C(=O)O)ccc1NC(=O)[C@@H]1NC[C@](C#N)(c2ccc(Cl)cc2F)[C@H]1c1cccc(Cl)c1F]

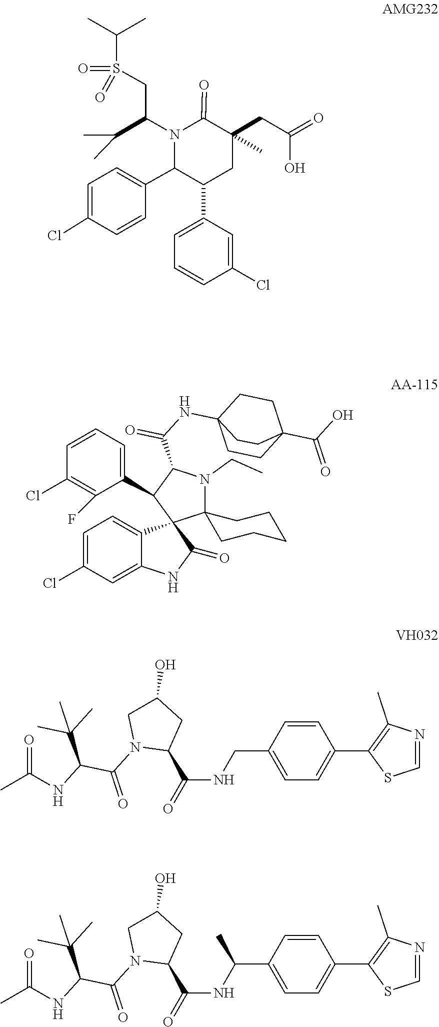

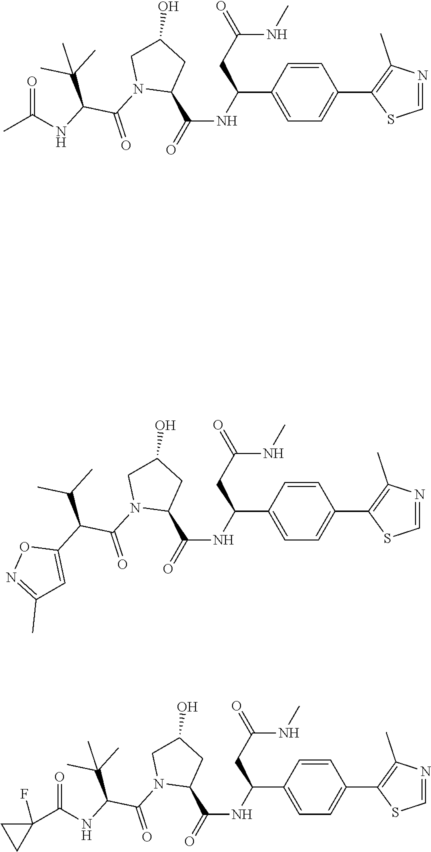 [SMILES: CNC(=O)C[C@H](NC(=O)[C@@H]1C[C@@H](O)CN1C(=O)[C@@H](NC(C)=O)C(C)(C)C)c1ccc(-c2scnc2C)cc1 ; CNC(=O)C[C@H](NC(=O)[C@@H]1C[C@@H](O)CN1C(=O)[C@@H](c1cc(C)no1)C(C)C)c1ccc(-c2scnc2C)cc1 ; CNC(=O)C[C@H](NC(=O)[C@@H]1C[C@@H](O)CN1C(=O)[C@@H](NC(=O)C1(F)CC1)C(C)(C)C)c1ccc(-c2scnc2C)cc1]

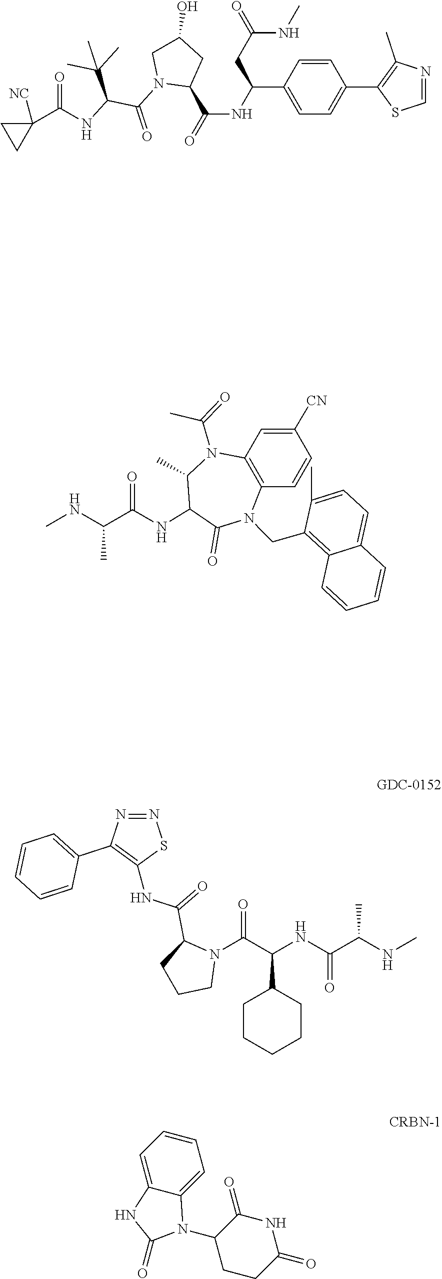 [SMILES: CNC(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1 ; CN[C@@H](C)C(=O)NC1C(=O)N(Cc2c(C)ccc3ccccc23)c2ccc(C#N)cc2N(C(C)=O)[C@H]1C ; CN[C@@H](C)C(=O)N[C@H](C(=O)N1CCC[C@H]1C(=O)Nc1snnc1-c1ccccc1)C1CCCCC1 ; O=C1CCC(n2c(=O)[nH]c3ccccc32)C(=O)N1]

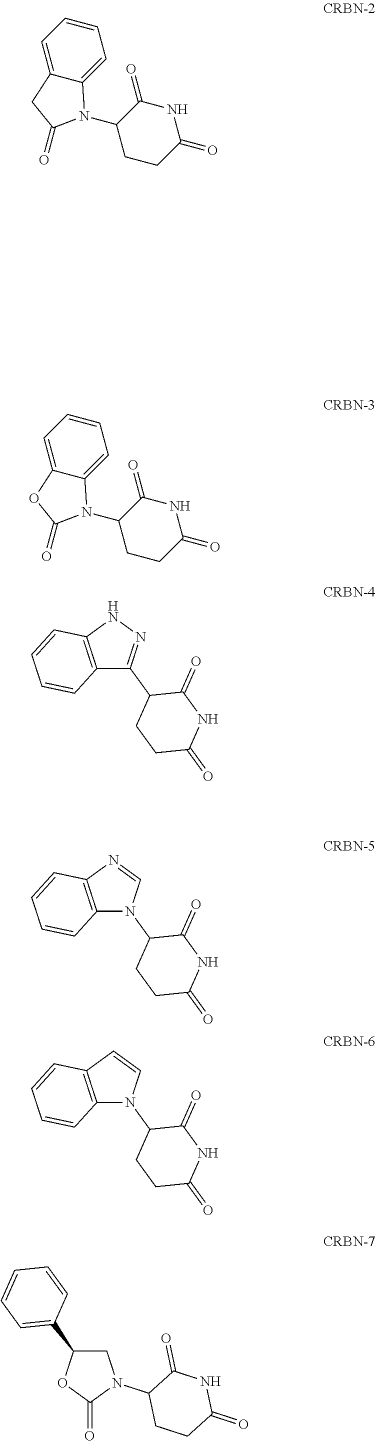 [SMILES: O=C1CCC(N2C(=O)Cc3ccccc32)C(=O)N1 ; O=C1CCC(n2c(=O)oc3ccccc32)C(=O)N1 ; O=C1CCC(c2n[nH]c3ccccc23)C(=O)N1 ; O=C1CCC(n2cnc3ccccc32)C(=O)N1 ; O=C1CCC(n2ccc3ccccc32)C(=O)N1 ; O=C1CCC(N2C[C@H](c3ccccc3)OC2=O)C(=O)N1]

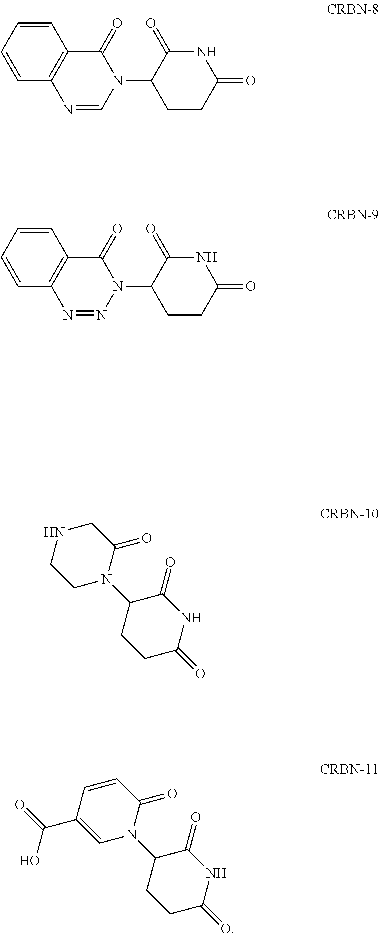 [SMILES: O=C1CCC(n2cnc3ccccc3c2=O)C(=O)N1 ; O=C1CCC(n2nnc3ccccc3c2=O)C(=O)N1 ; O=C1CCC(N2CCNCC2=O)C(=O)N1 ; O=C1CCC(n2cc(C(=O)O)ccc2=O)C(=O)N1]

In another embodiment, the degradation tag is derived from any of the following: thalidomide, pomalidomide, lenalidomide, CRBN-1, CRBN-2, CRBN-3, CRBN-4, CRBN-5, CRBN-6, CRBN-7, CRBN-8, CRBN-9, CRBN-10, and CRBN-11.

In another embodiment, the degradation tag is selected from the group consisting of:

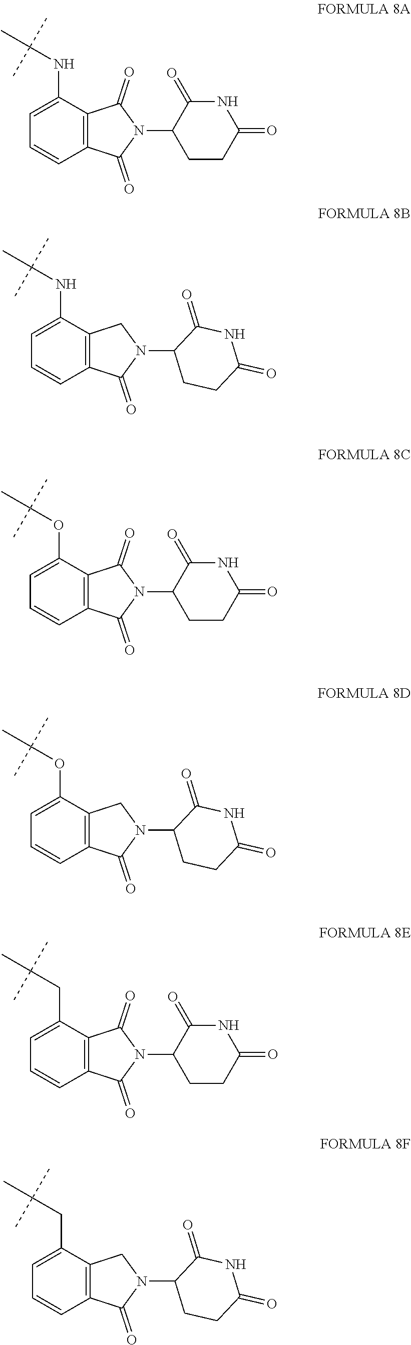

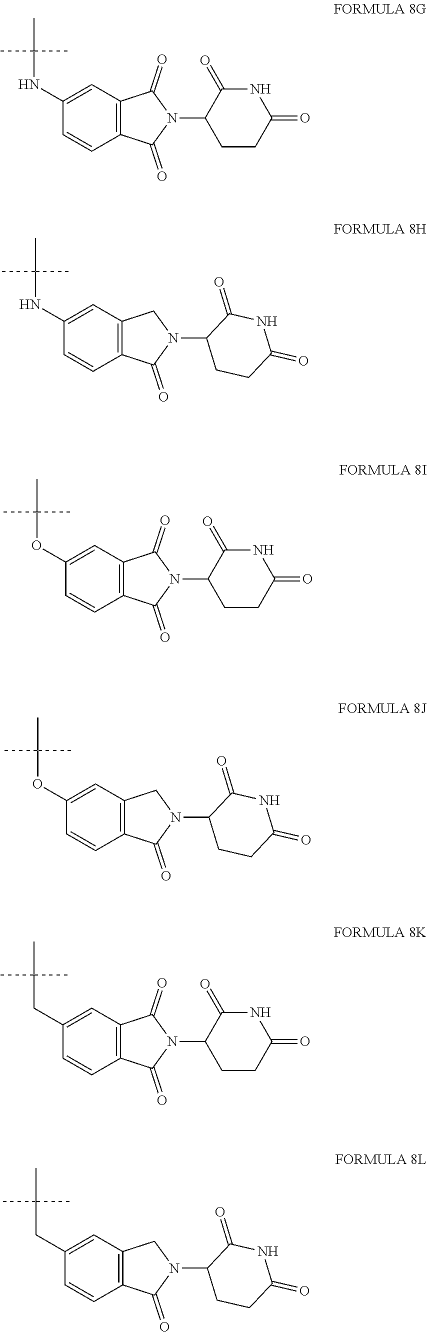

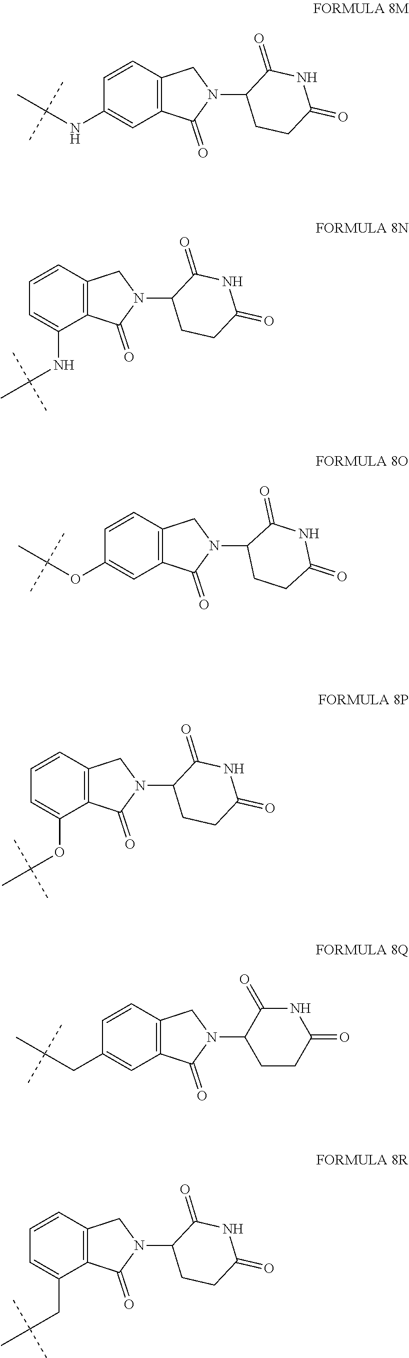

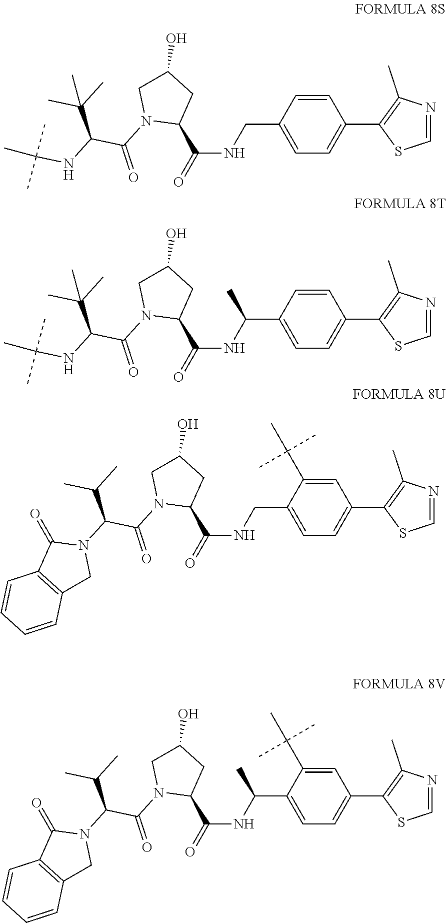

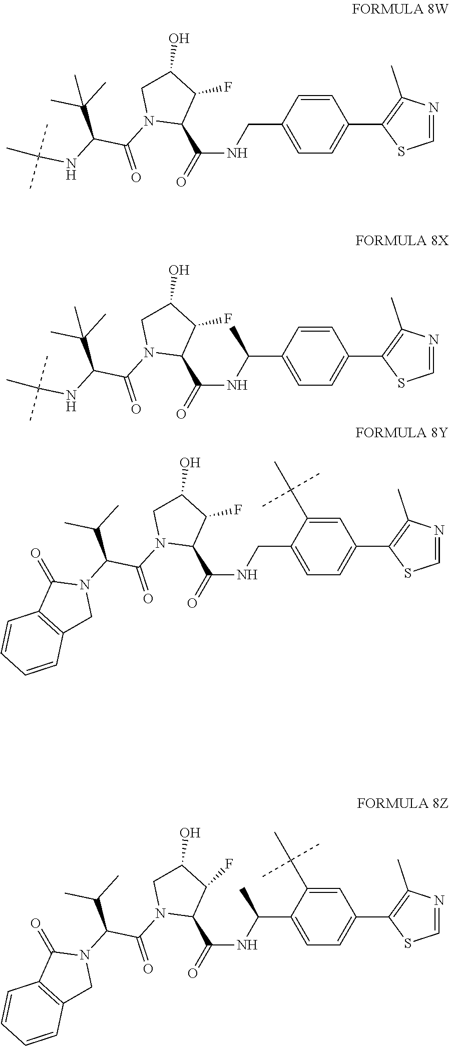

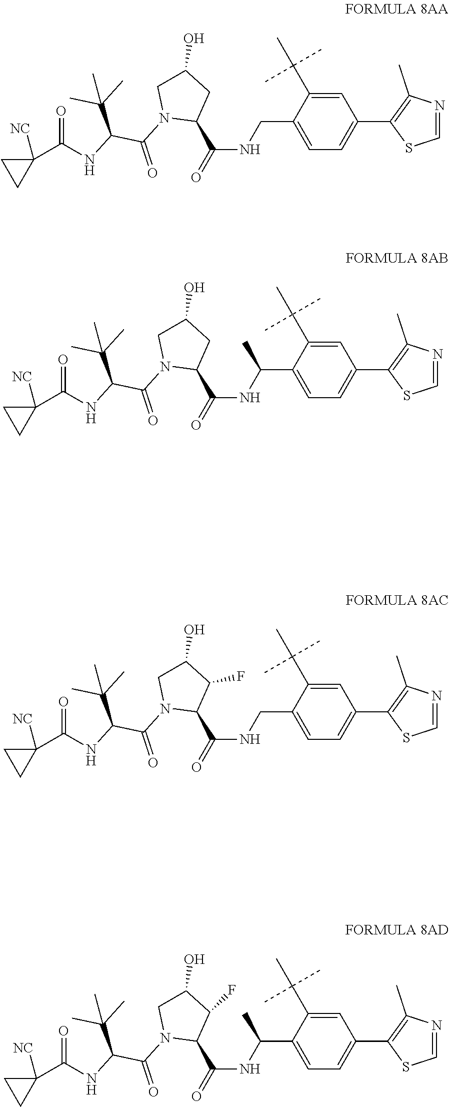

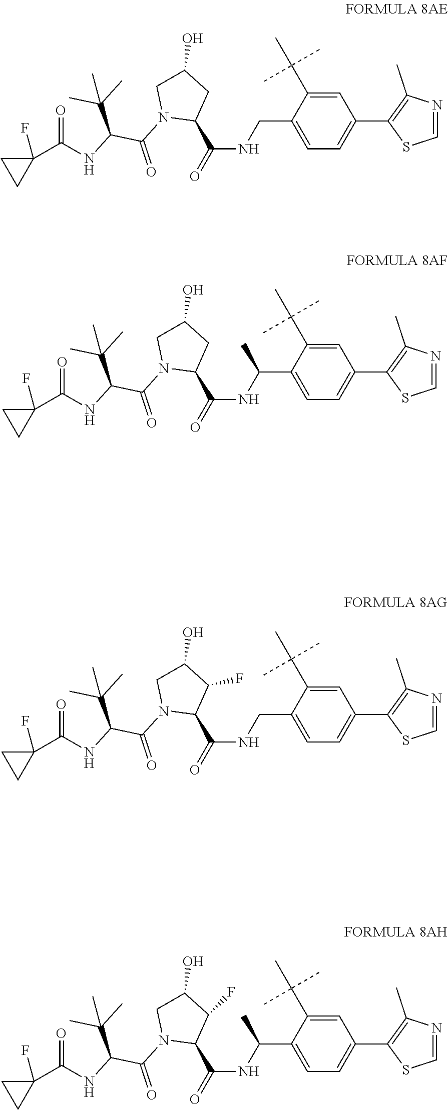

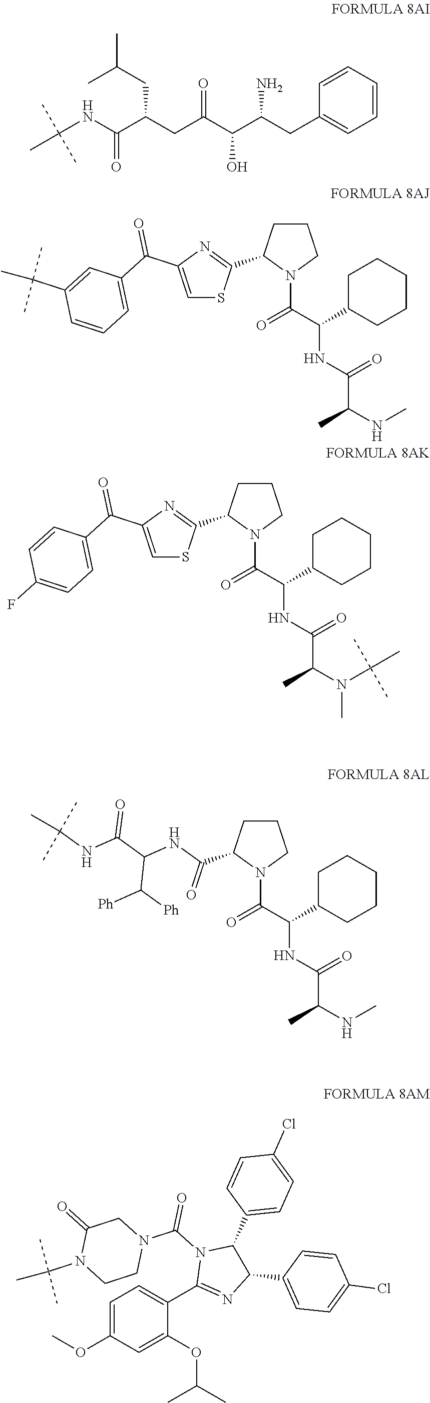

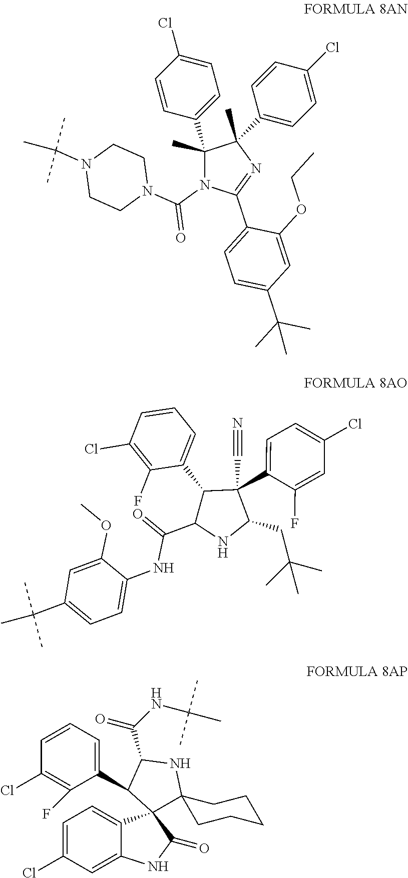

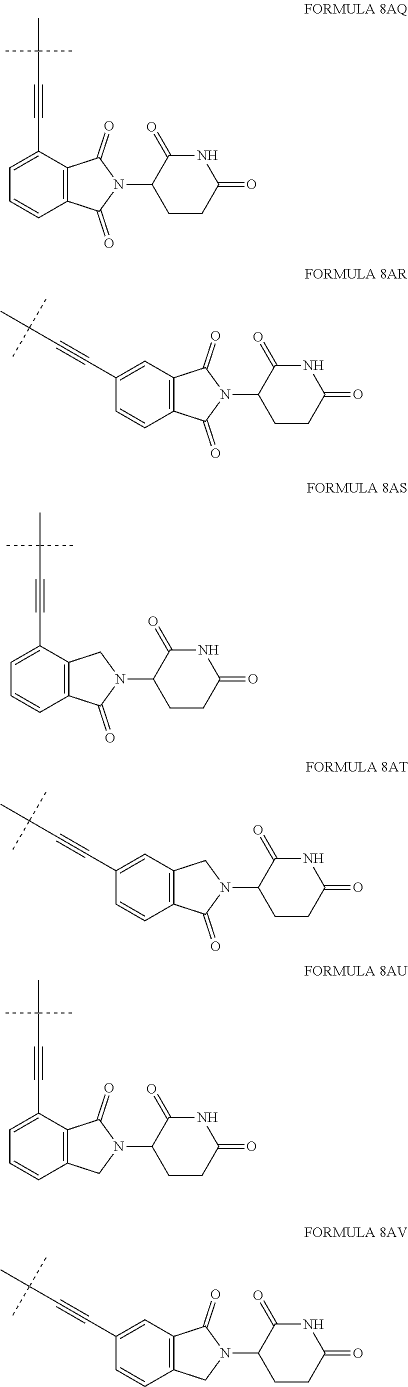

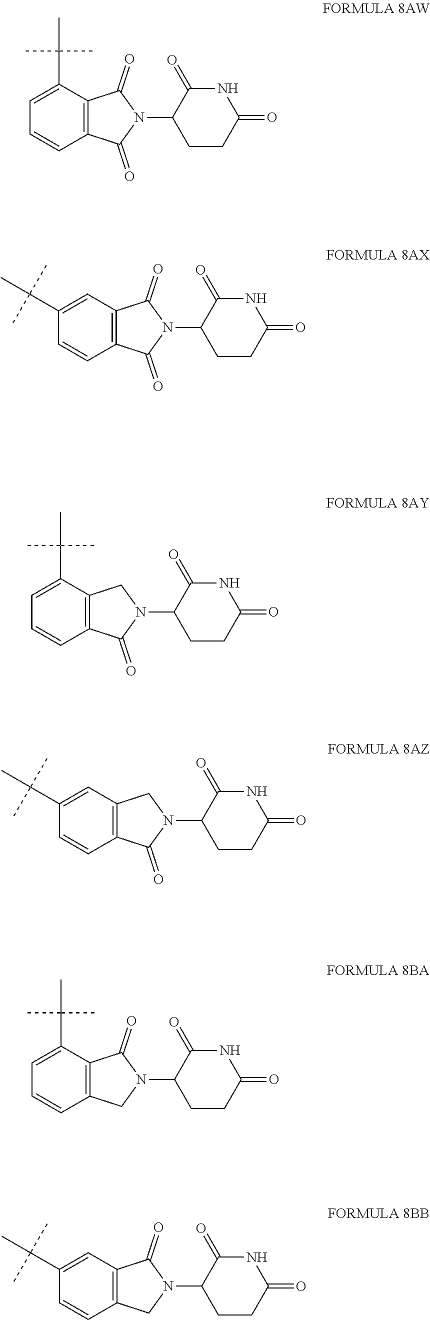

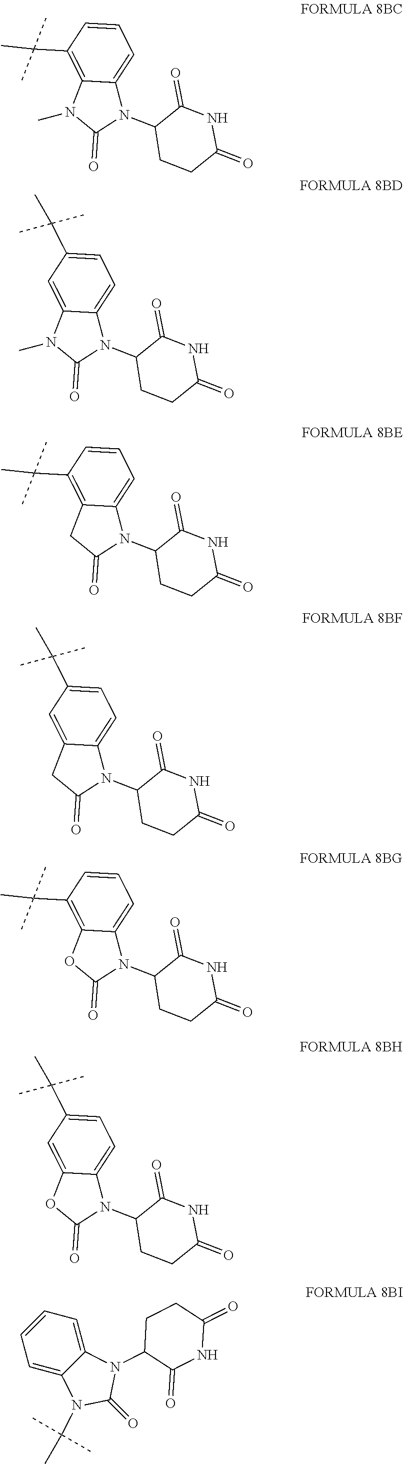

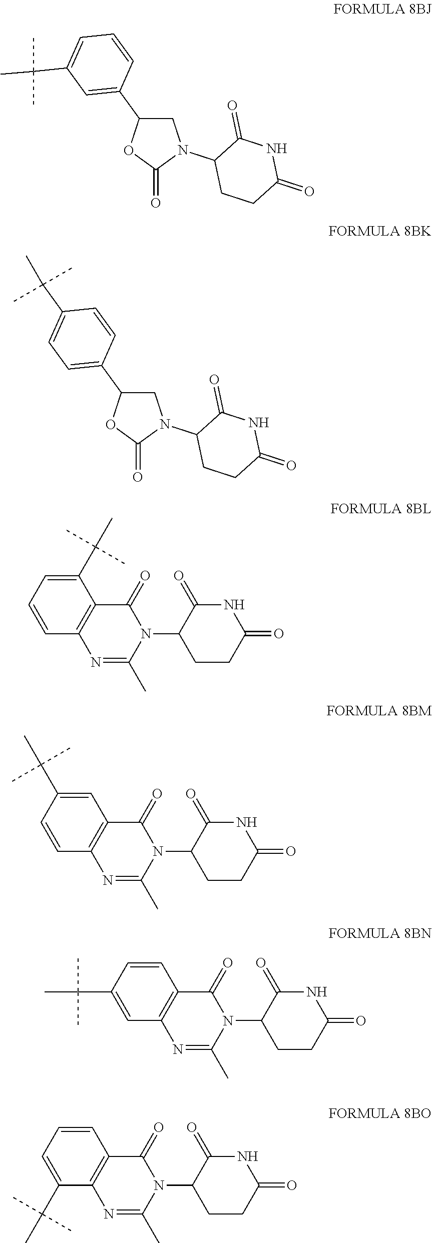

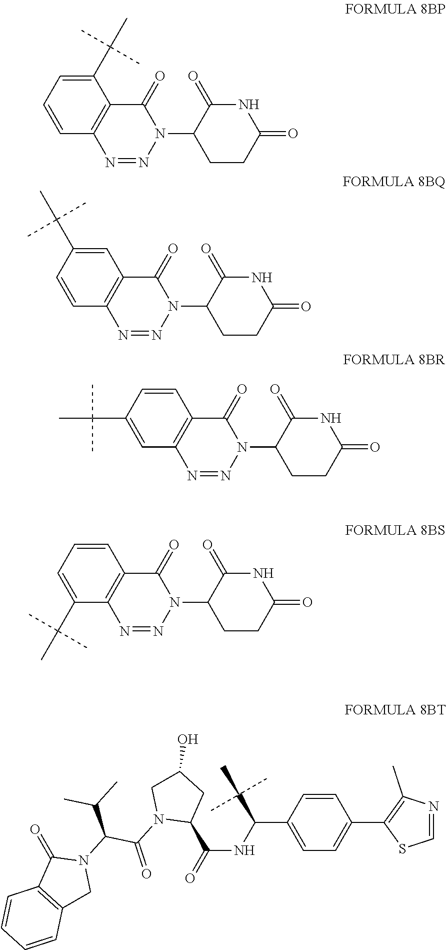

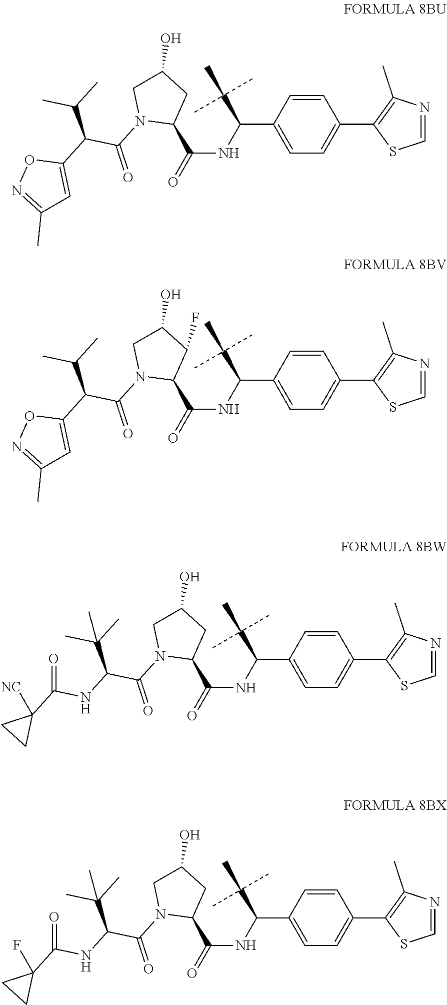

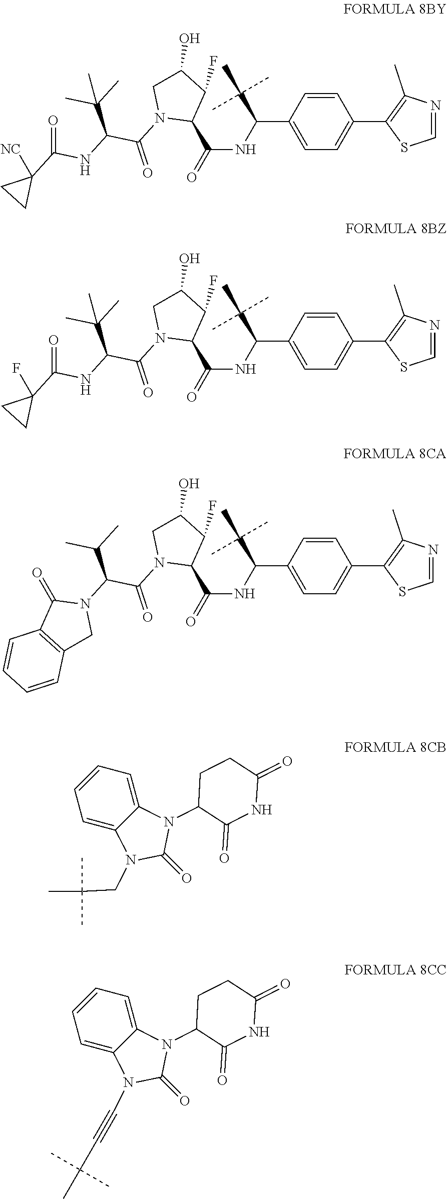

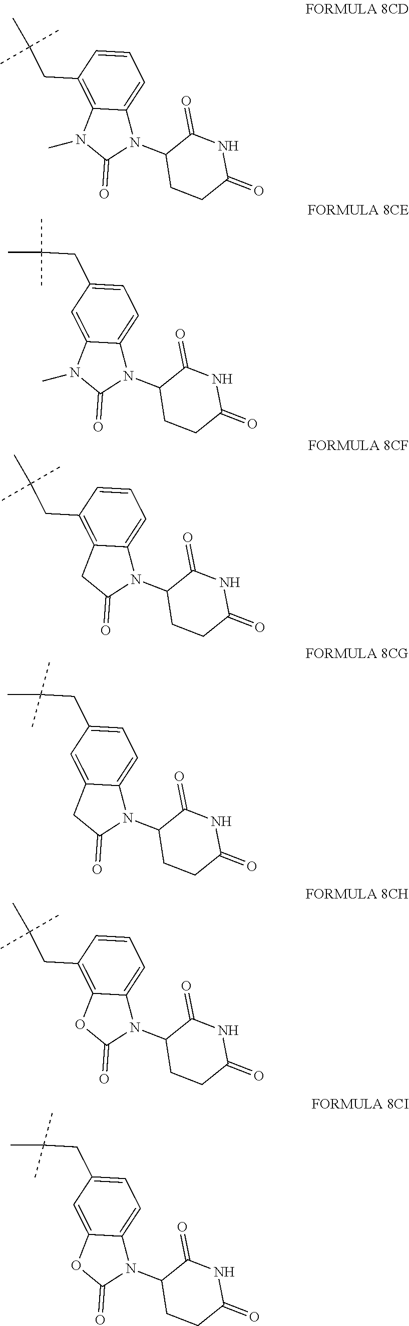

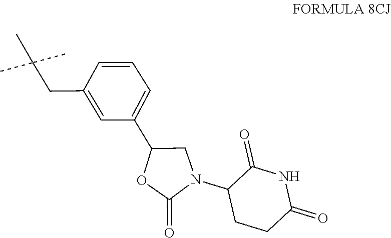

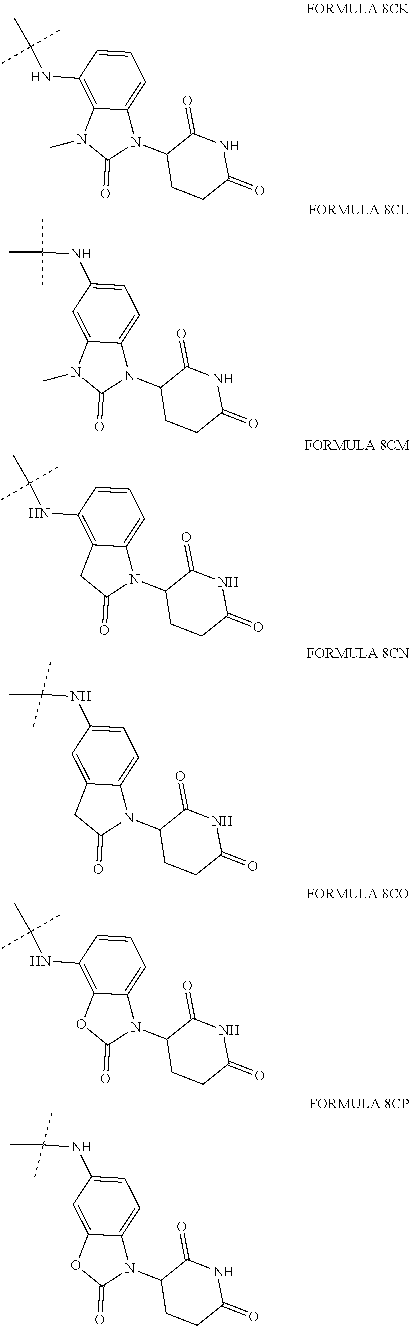

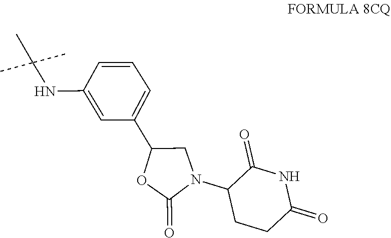

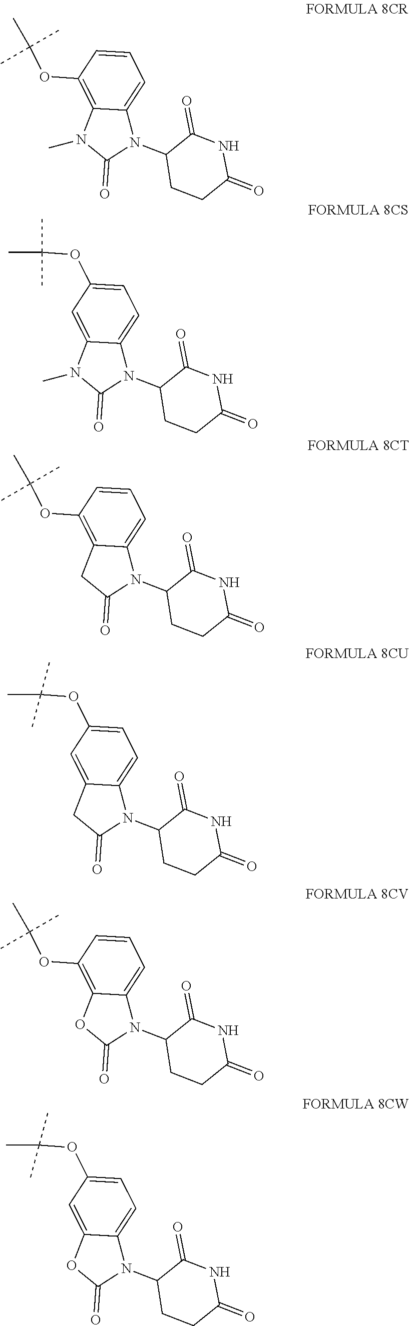

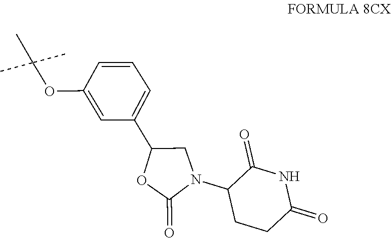

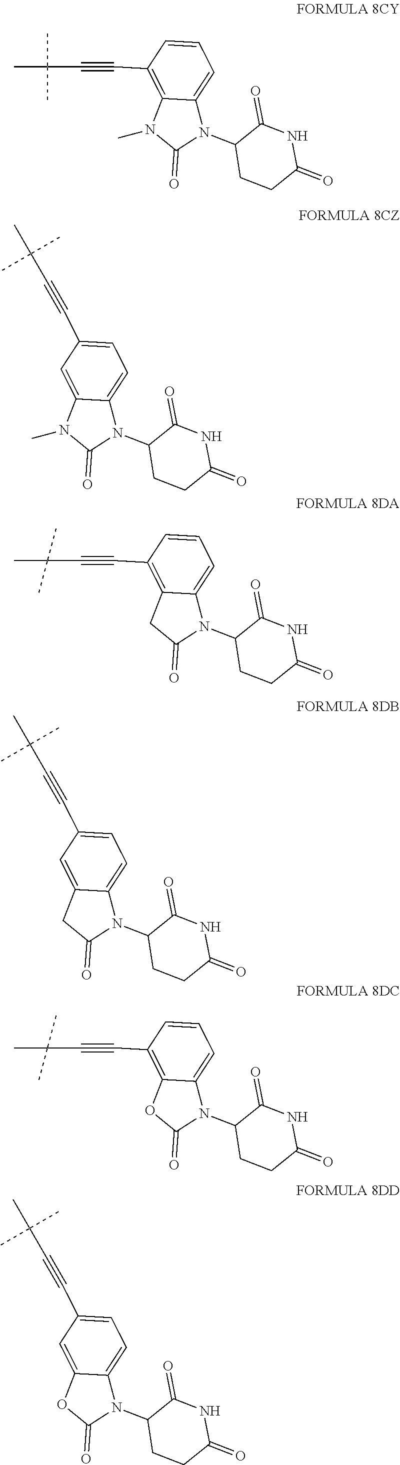

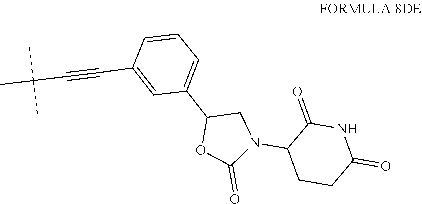

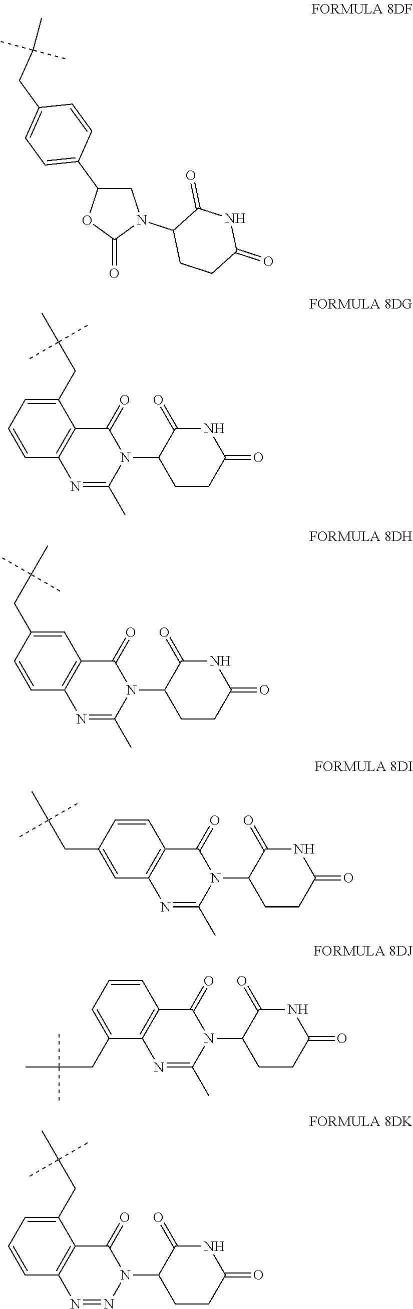

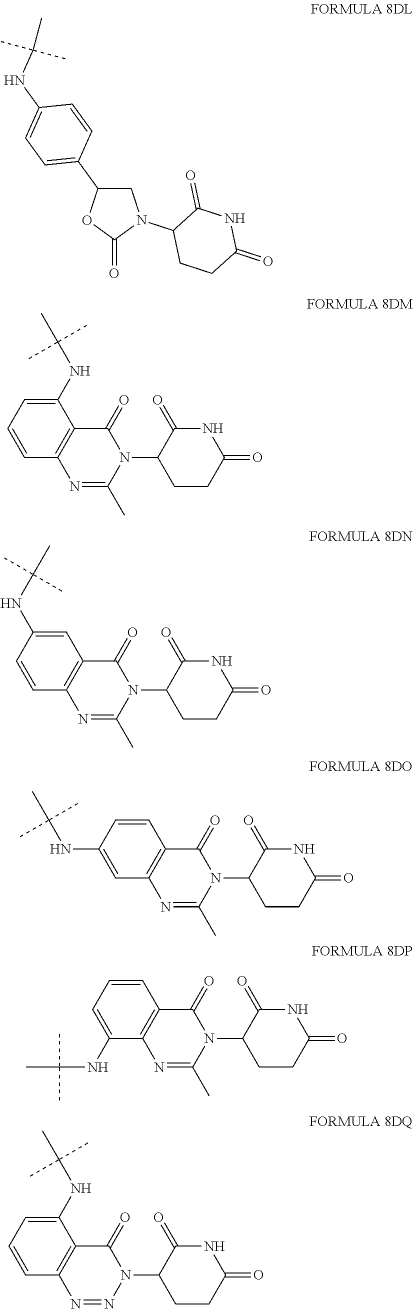

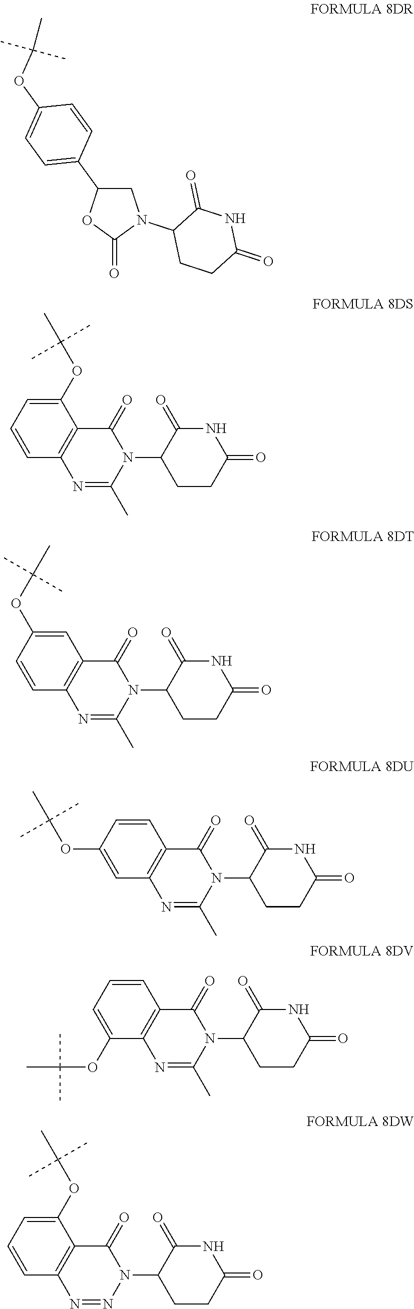

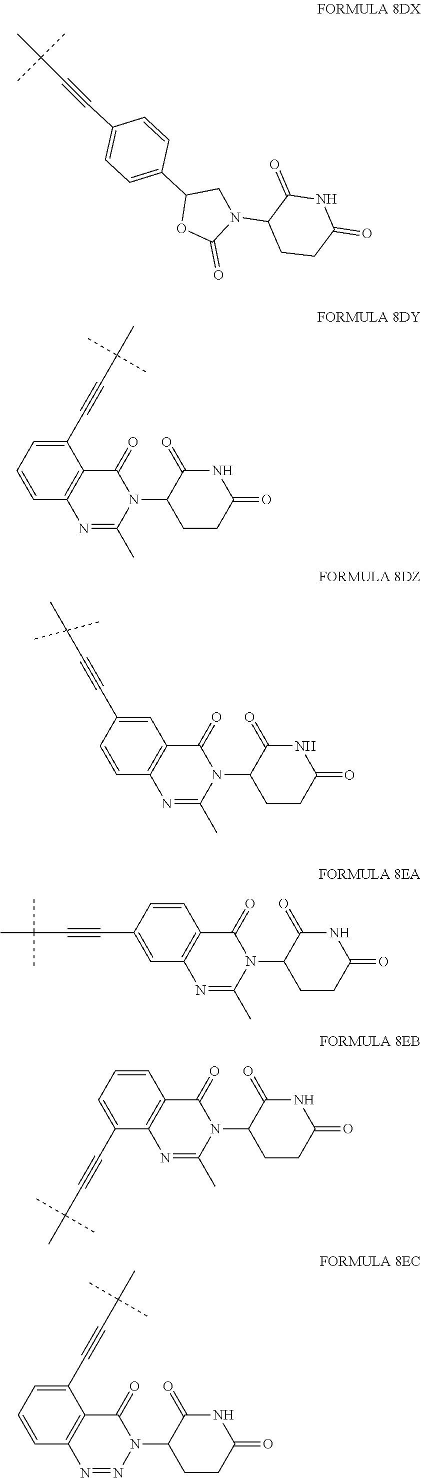

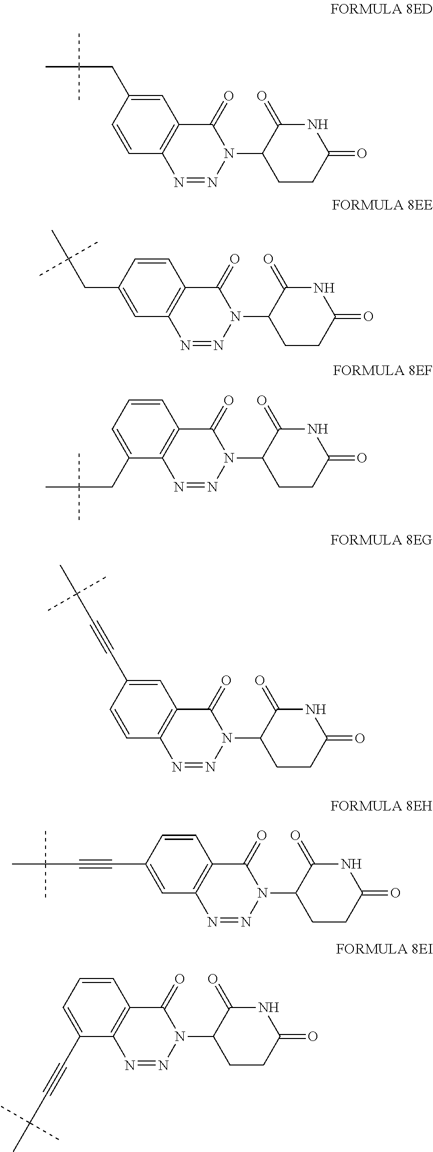

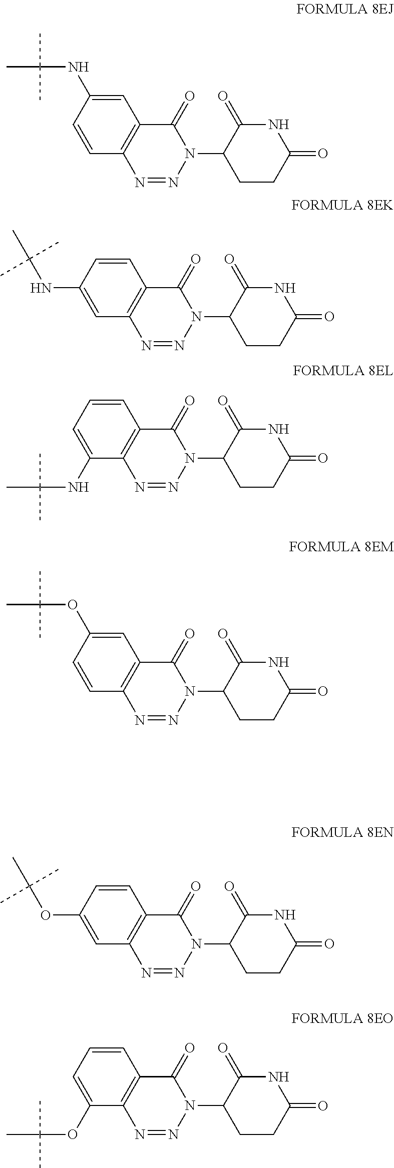

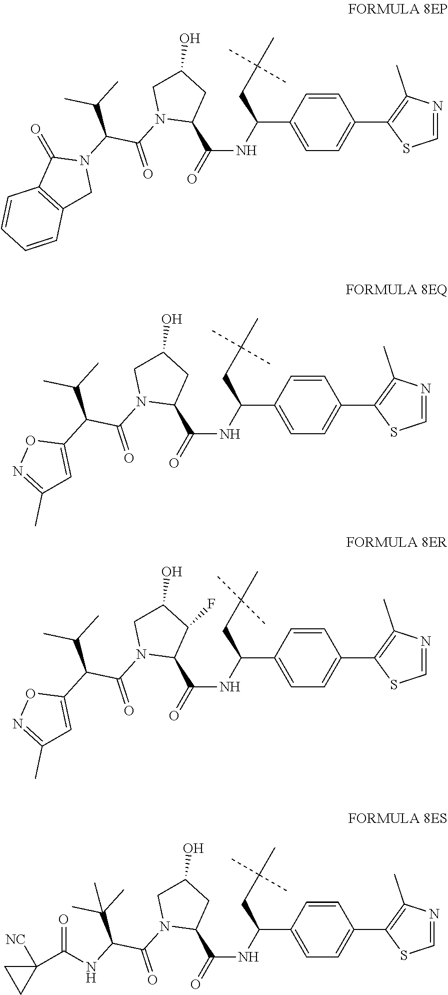

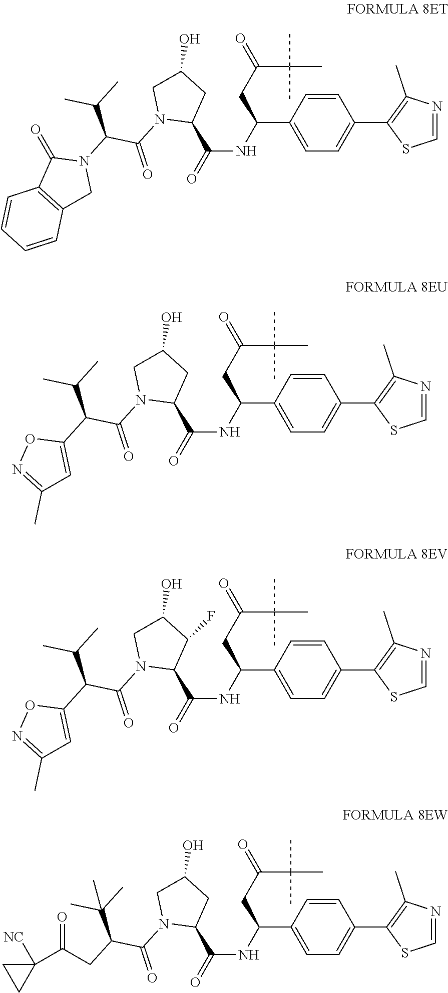

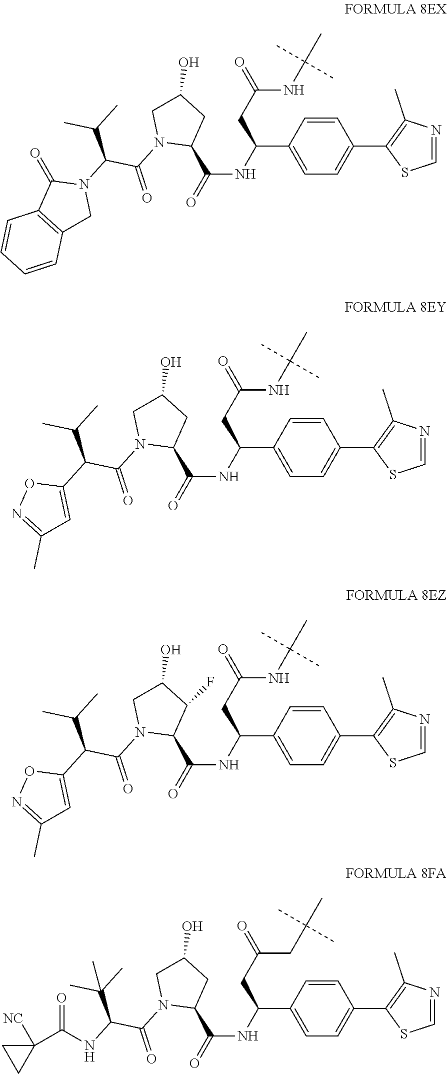

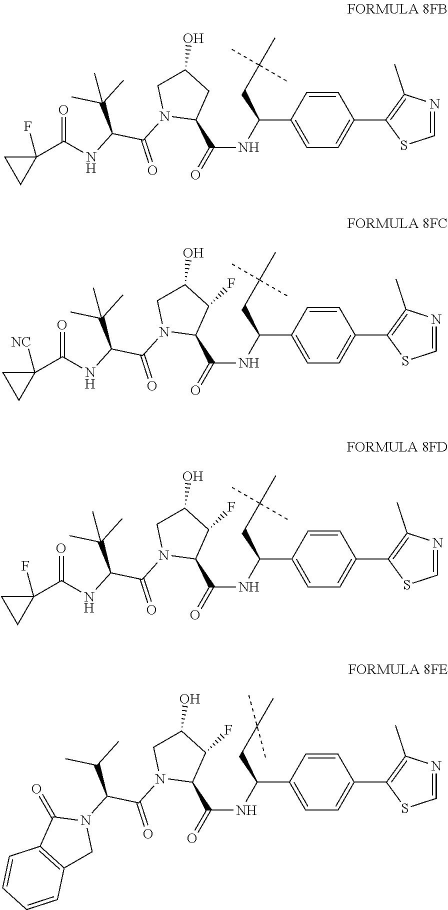

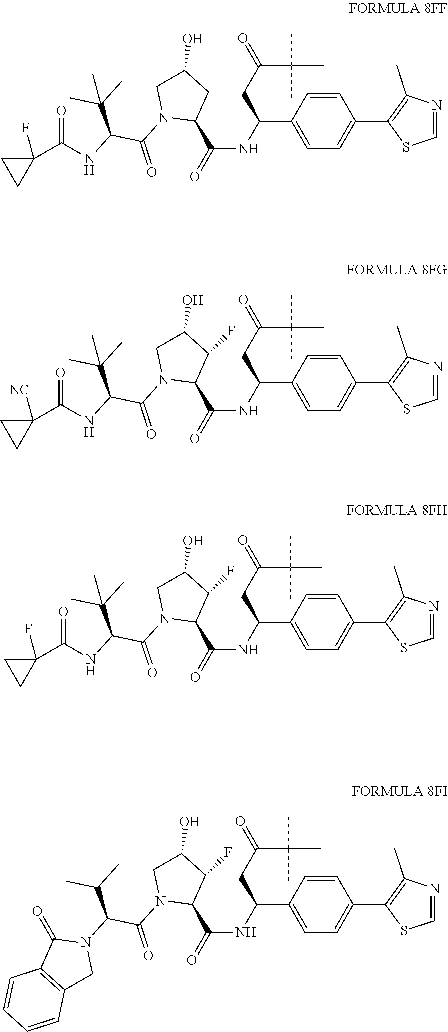

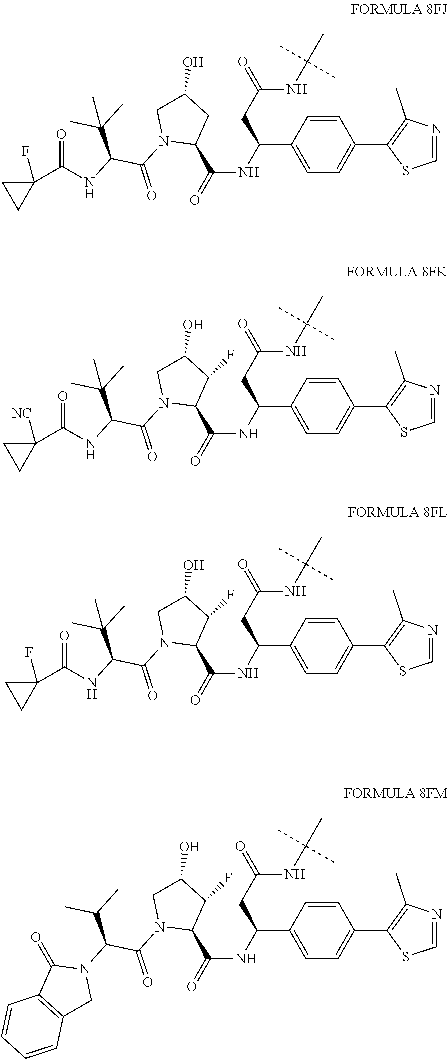

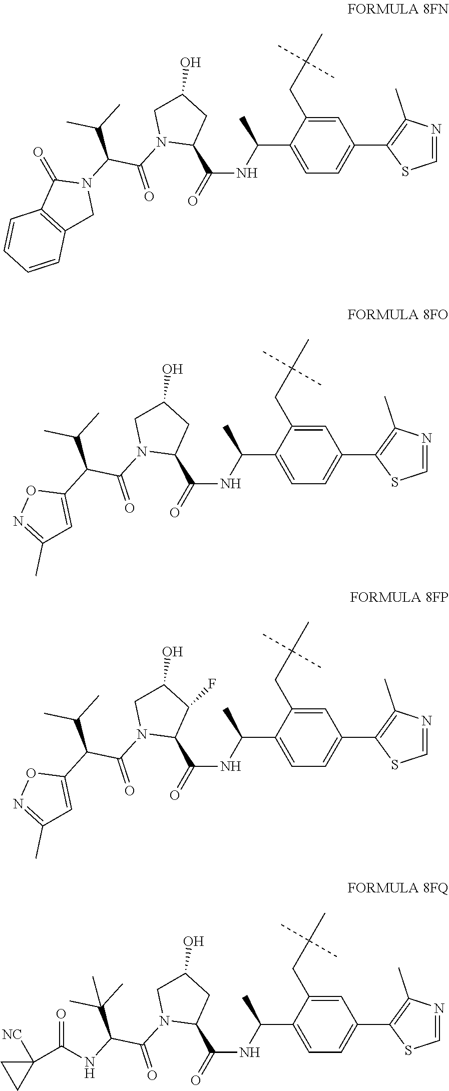

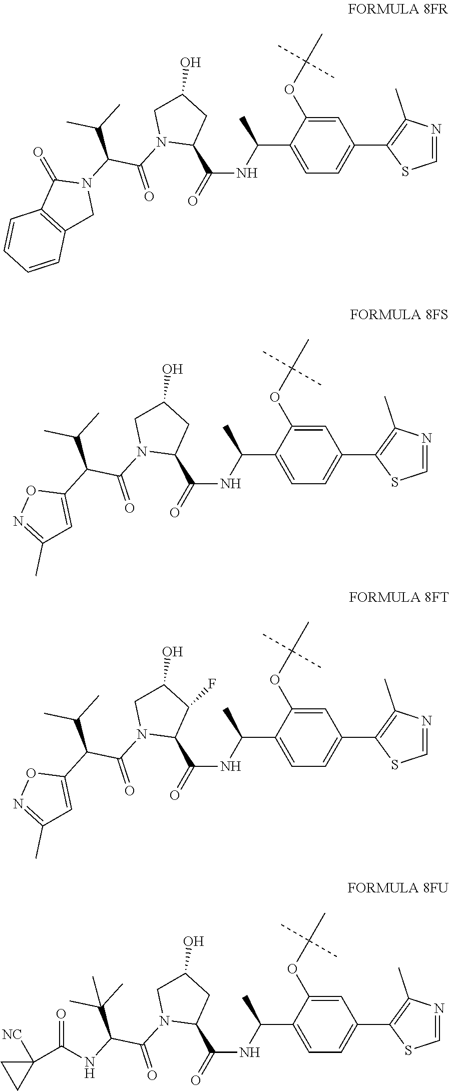

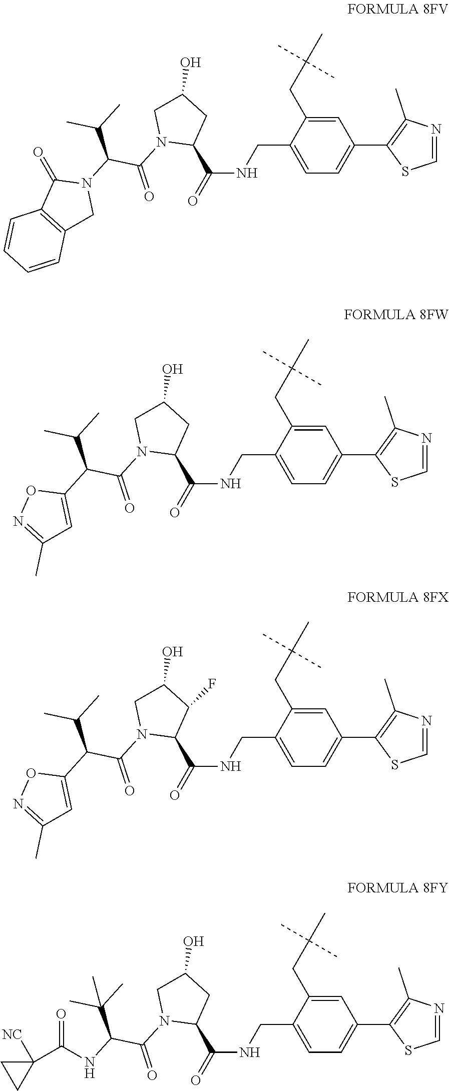

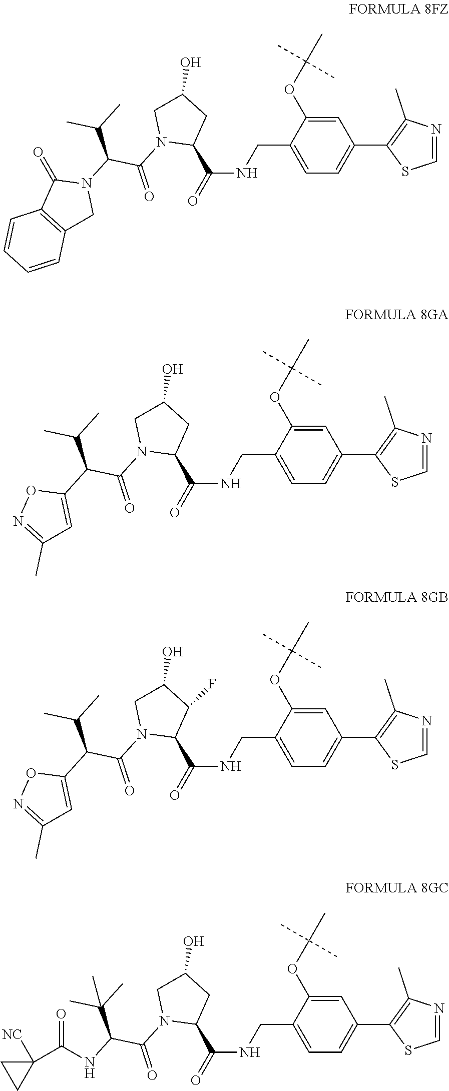

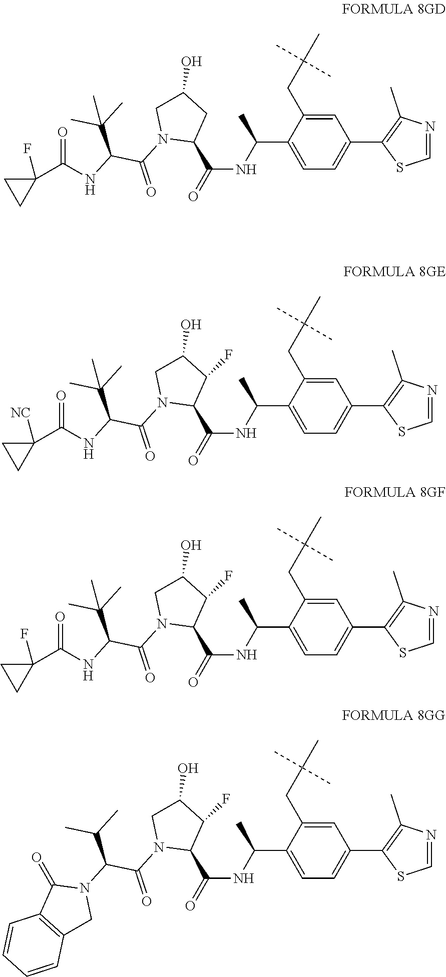

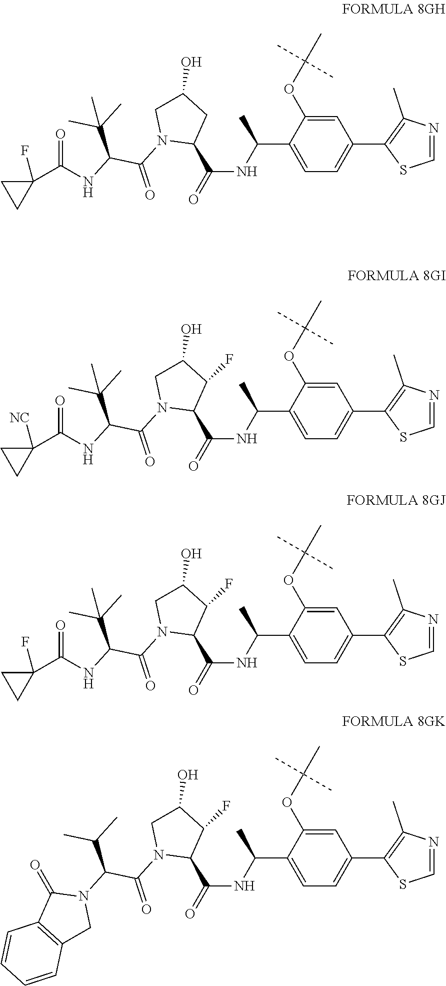

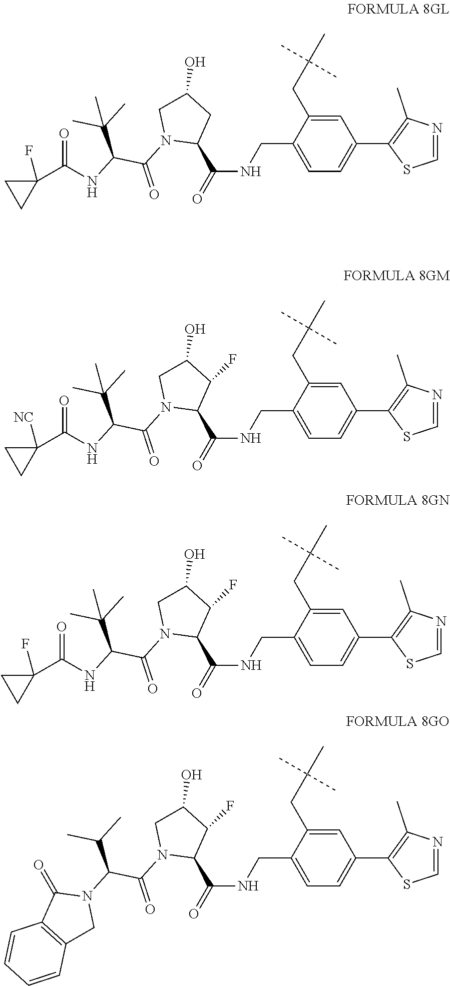

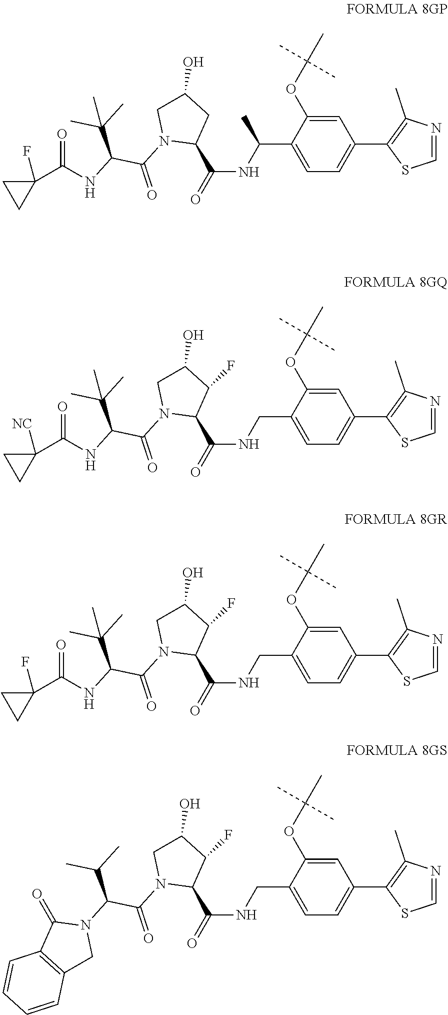

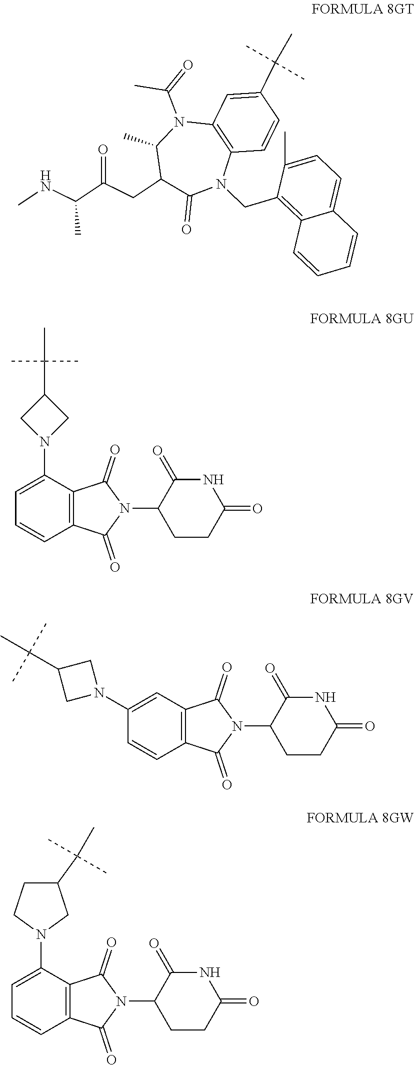

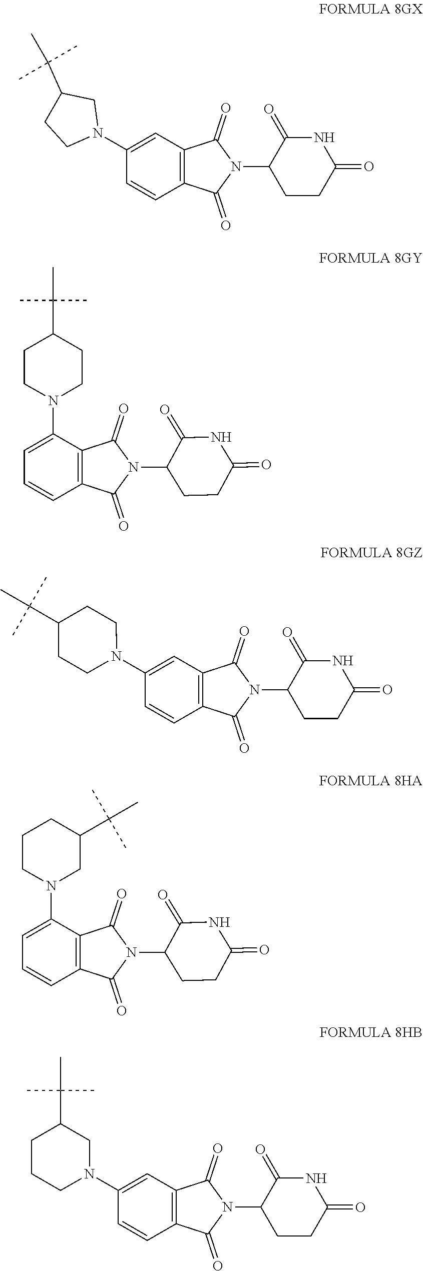

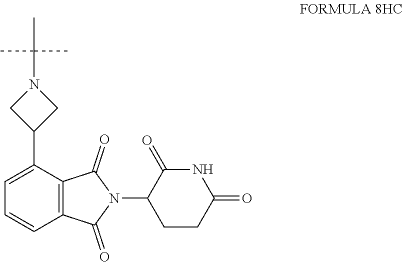

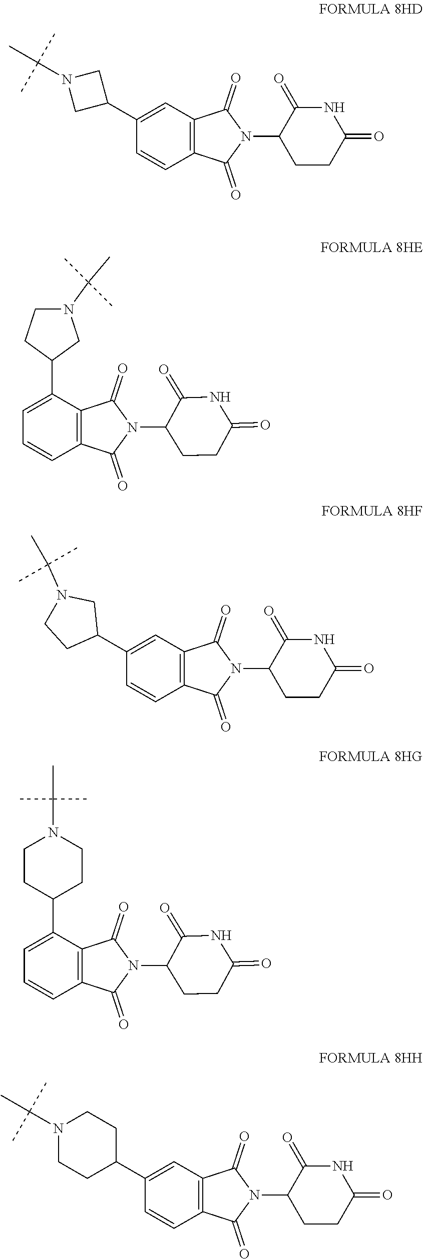

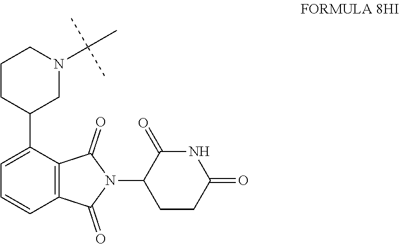

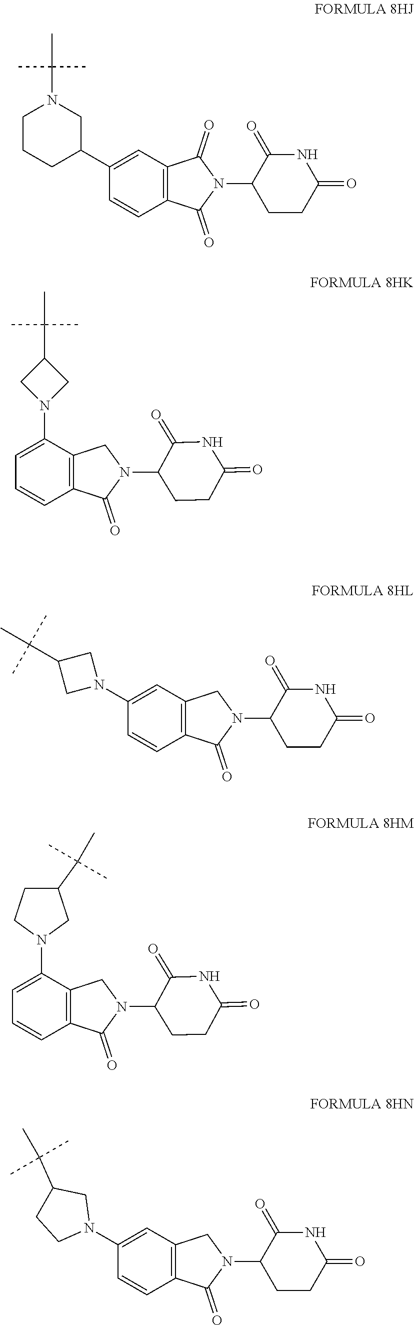

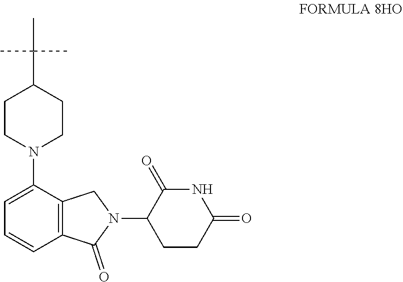

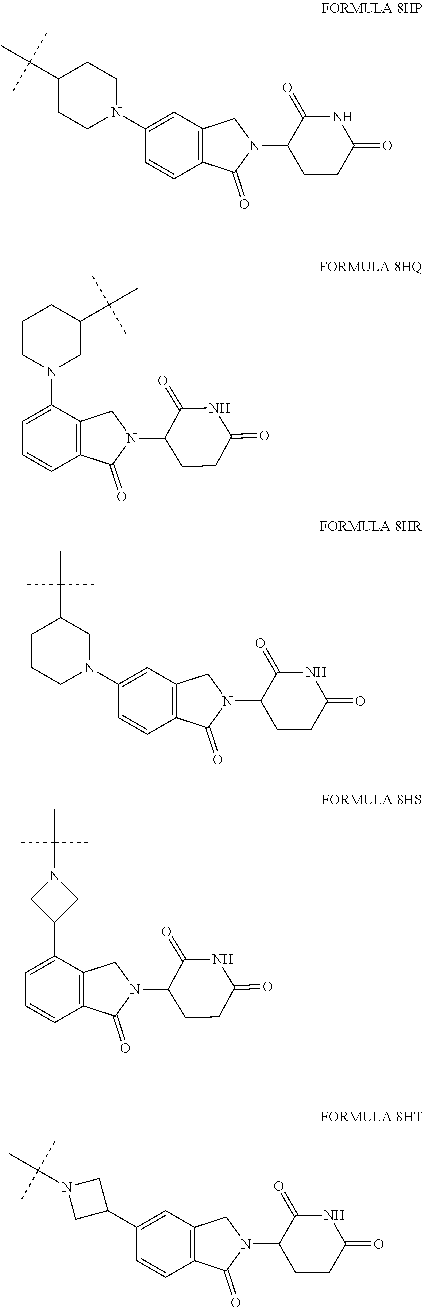

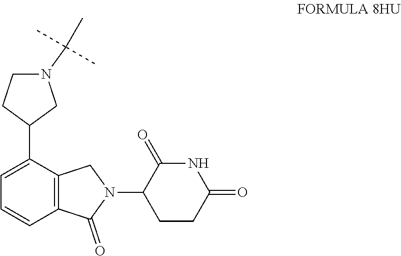

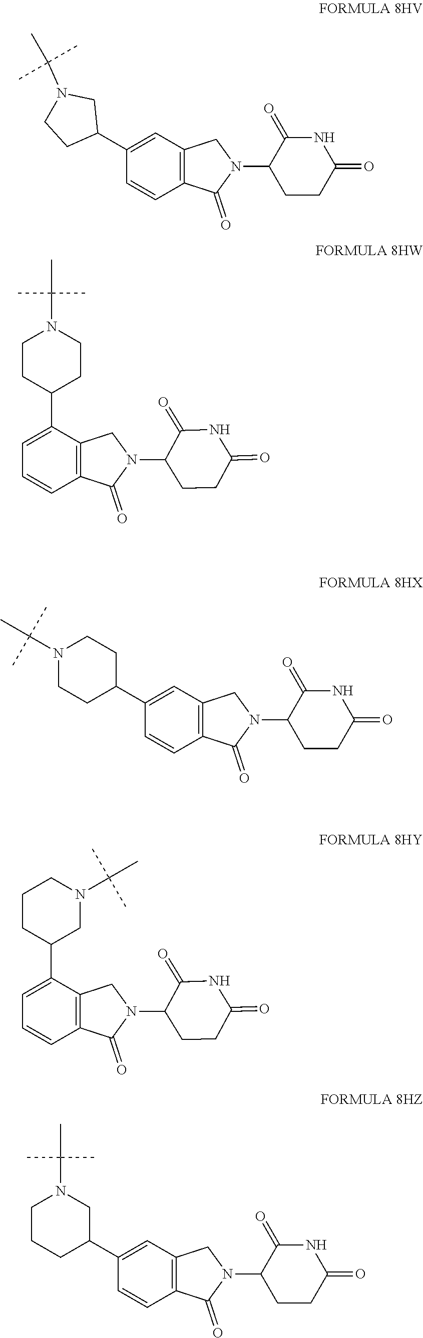

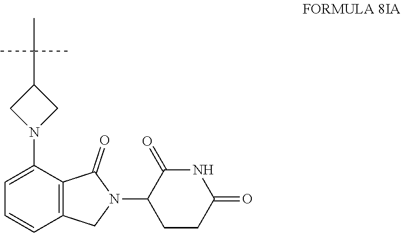

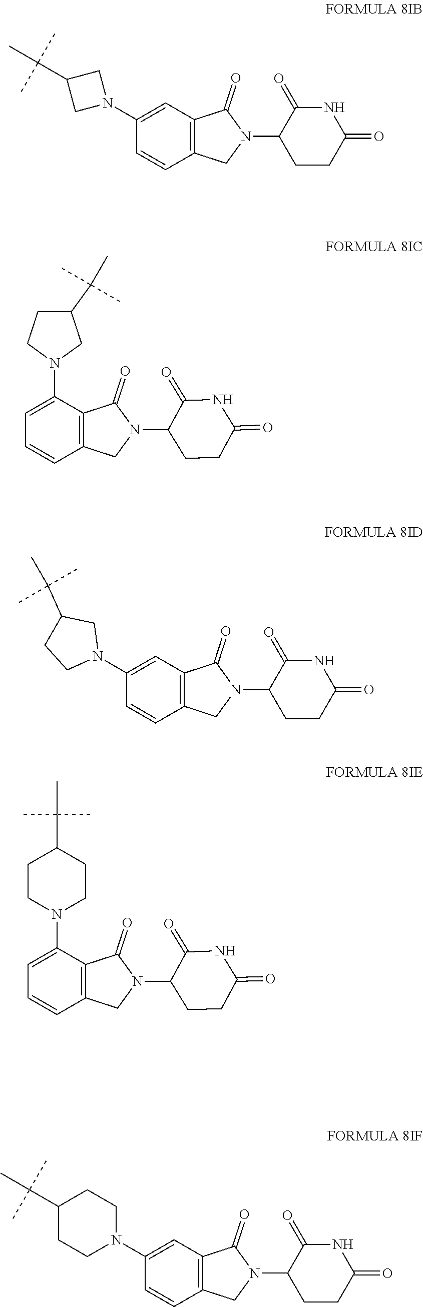

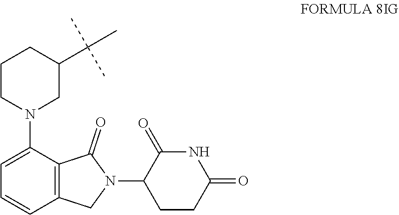

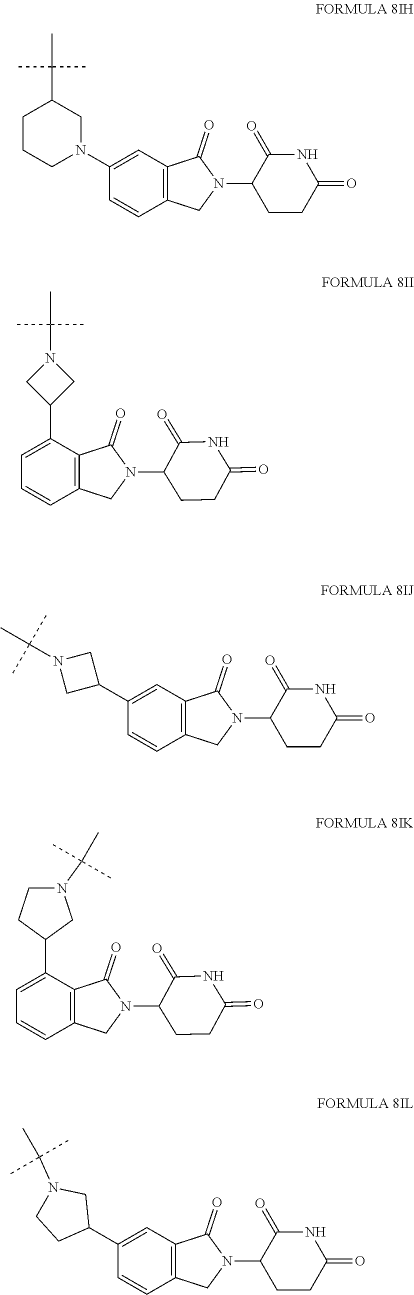

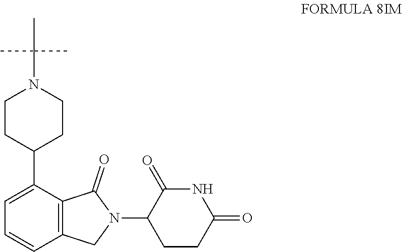

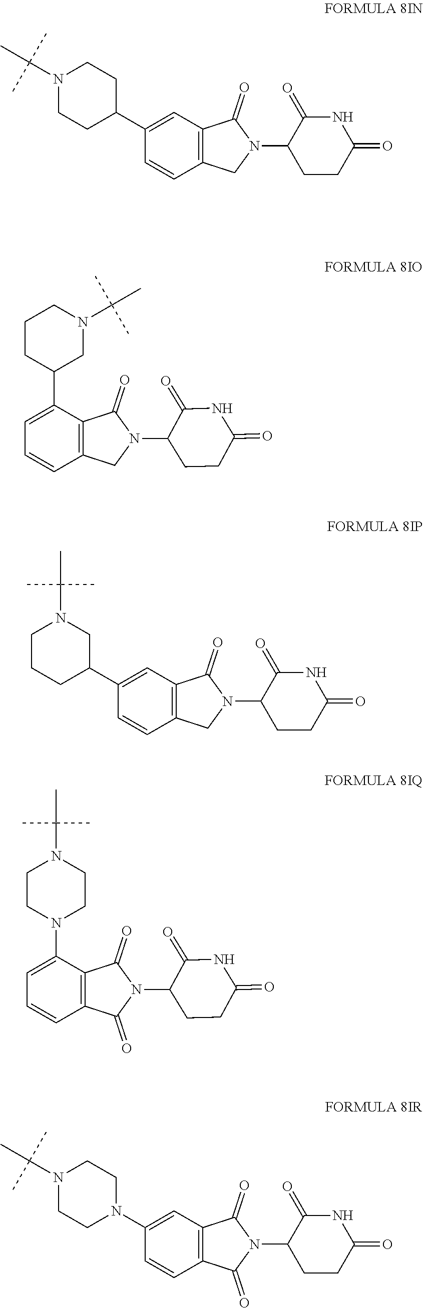

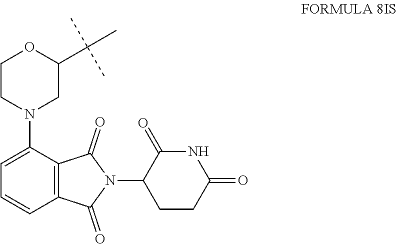

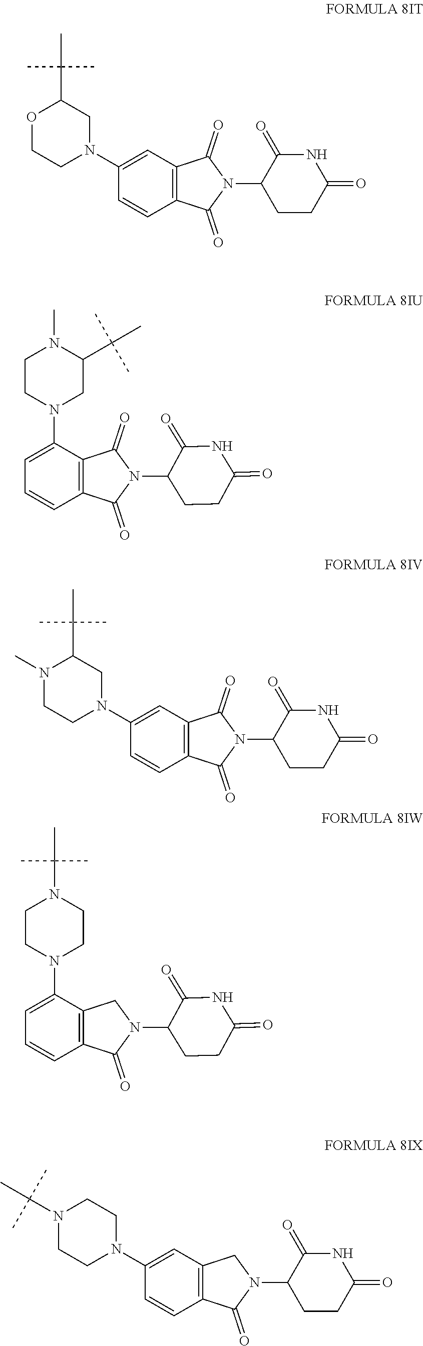

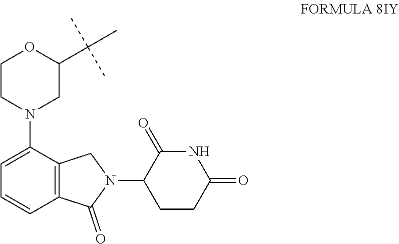

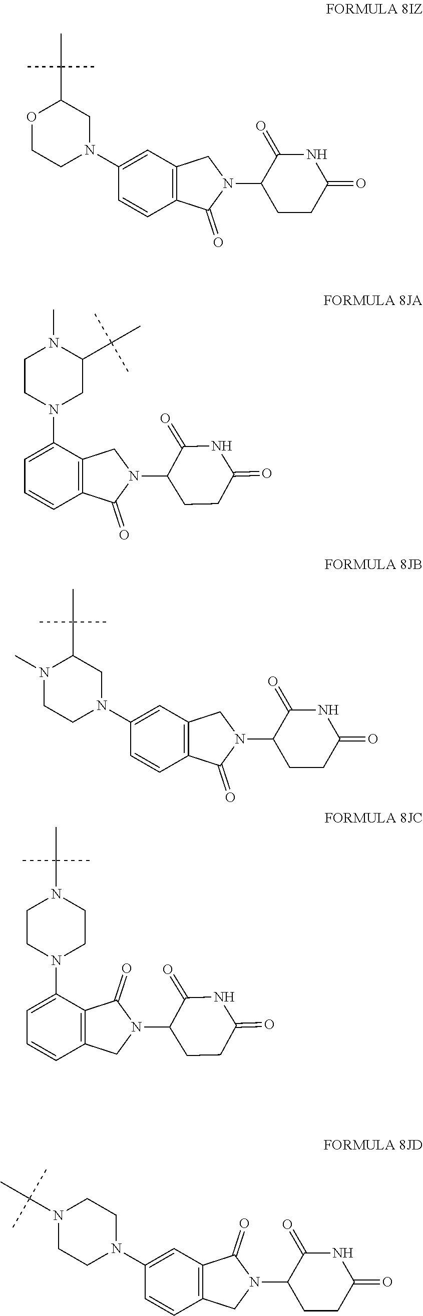

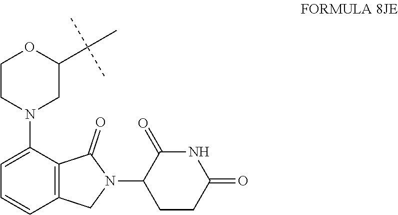

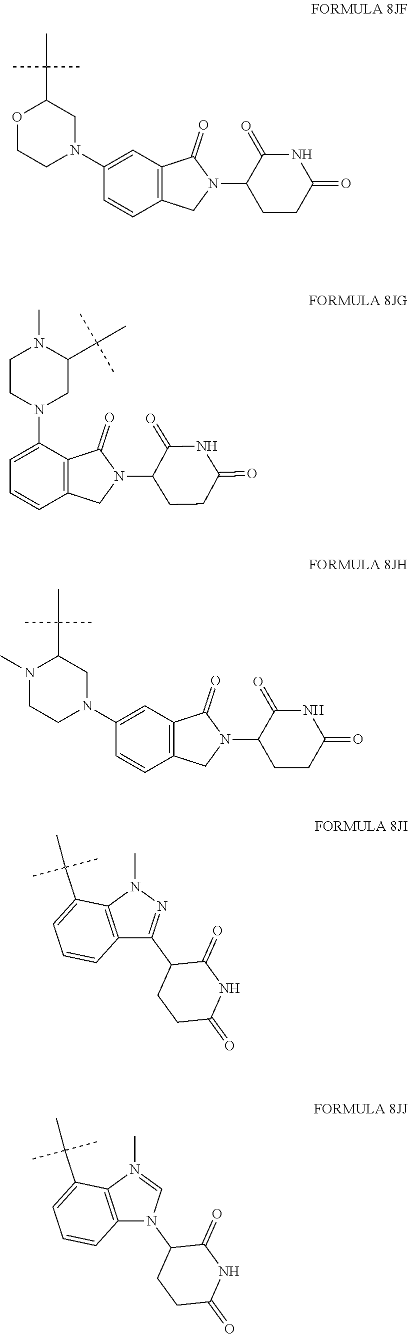

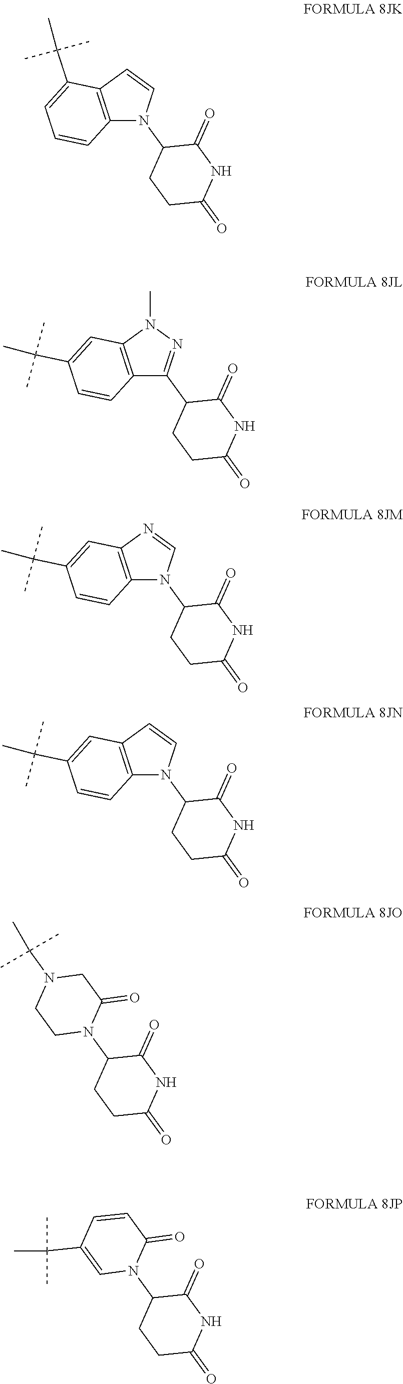

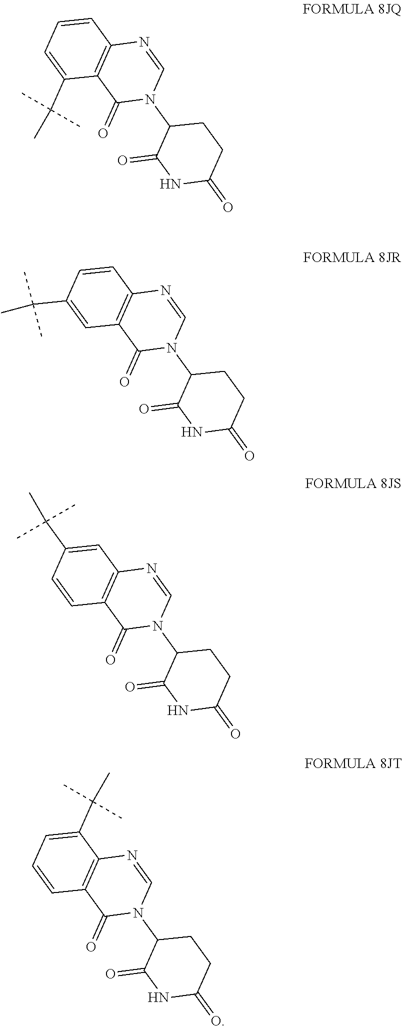

In some embodiments, the degradation tag is selected from FORMULAE 8A, 8B, 8C, 8D, 8E, 8F, 8G, 8H, 8I, 8G, 8K, 8L, 8M, 8N, 8O, 8P, 8Q, 8R, 8AQ, 8AR, 8AS, 8AT, 8AU, 8AV, 8AW, 8AX, 8AY, 8AZ, 8BA, 8BB, 8BC, 8BD, 8BI, 8CB, 8CC, 8CD, 8CE, 8CK, 8CL, 8CR, 8CS, 8CY, 8CZ, 8GU, 8GV, 8GW, 8GX, 8GY, 8GZ, 8HA, 8HB, 8HC, 8HD, 8HE, 8HF, 8HG, 8HH, 8HI, 8HJ, 8HK, 8HL, 8HM, 8HN, 8HO, 8HP, 8HQ, 8HR, 8HS, 8HT, 8HU, 8HV, 8HW, 8HX, 8HY, 8HZ, 8IA, 8IB, 8IC, 8ID, 81E, 8IF, 8IG, 8IH, 8II, 8IJ, 8IK, 8IL, 8IM, 8IN, 8IO, 8IP, 8IQ, 8IR, 8IS, 8IT, 8IU, 8IV, 8IW, 8IX, 8IY, 8IZ, 8JA, 8JB, 8JC, 8JD, 8JE, 8JF, 8JG, 8JH, 8JI, and 8JJ.

In some embodiments, the degradation tag is selected from FORMULAE 8G, 8H, 8I, 8J, 8K, 8L, 8M, 8O, 8Q, 8AR, 8AT, 8AV, 8AX, 8AZ, 8BB, 8BC, 8BD, 8BI, 8CB, 8CC, 8CD, 8CE, 8CK, 8CL, 8CR, 8CS, 8CY, 8CZ, 8GV, 8GX, 8GZ, 8HD, 8HF, 8HH, 8HL, 8HN, 8HP, 8HT, 8HV, 8HX, 8IB, 8ID, 8IF, 8IJ, 8IL, 8IN, 8IR, 8IX, and 8JD.

In some embodiments, the linker moiety is of FORMULA 9:

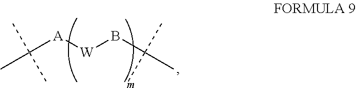

wherein

A, W, and B, at each occurrence, are independently selected from null, or bivalent moiety selected from R′—R″, R′COR″, R′CO₂R″, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R¹)R″, R′N(R¹)R″, R′N(R¹)COR″, R′N(R¹)C(O)OR″, R′N(R¹)CON(R²)R″, R′N(R¹)C(S)R″, R′N(R¹)S(O)R″, R′N(R¹)S(O)₂R″, R′N(R¹)S(O)₂N(R²)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R¹ and R², R′ and R¹, R′ and R², R″ and R¹, R″ and R² together with the atom to which they are connected form a 3-20 membered cycloalkyl or 4-20 membered heterocyclyl ring; and

m is 0 to 15.

In one embodiment, the linker moiety is of FORMULA 9A:

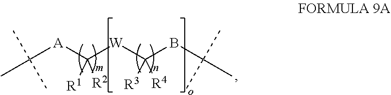

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W, and B, at each occurrence, are independently selected from null, or bivalent moiety selected from R′—R″, R′COR″, R′CO₂R″, R′C(O)N(R¹)R″, R′C(S)N(R⁵)R″, R′OR″, R′OC(O)R″, R′OC(O)OR″, R′OCON(R⁵)R″, R′SR″, R′SOR″, R′SO₂R″, R′SO₂N(R⁵)R″, R′N(R⁵)R″, R′N(R⁵)COR″, R′N(R⁵)C(O)OR″, R′N(R⁵)CON(R⁶)R″, R′N(R⁵)C(S)R″, R′N(R⁵)S(O)R″, R′N(R⁵)S(O)₂R″, R′N(R⁵)S(O)₂N(R⁶)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;

m is 0 to 15;

n, at each occurrence, is 0 to 15; and

o is 0 to 15.

In another embodiment, the linker moiety is of FORMULA 9B:

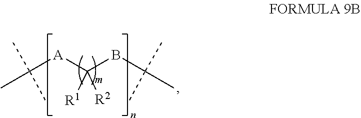

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or

R¹ and R² together with the atom to which they are connected form a 3-20 membered cycloalkyl or 4-20 membered heterocyclyl ring;

A and B, at each occurrence, are independently selected from null, or bivalent moiety selected from R′—R″, R′COR″, R′CO₂R″, R′C(O)N(R³)R″, R′C(S)N(R³)R″, R′OR″, R′OC(O)R″, R′OC(O)OR″, R′OCON(R³)R″, R′SR″, R′SOR″, R′SO₂R″, R′SO₂NR″R³, R′N(R³)R″, R′N(R³)COR″, R′N(R³)C(O)OR″, R′N(R³)CON(R⁴)R″, R′N(R³)C(S)R″, R′N(R³)S(O)R″, R′N(R³)S(O)₂R″, R′N(R³)S(O)₂N(R⁴)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁—C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;

each m is 0 to 15; and

n is 0 to 15.

In another embodiment, the linker moiety is of FORMULA 9C:

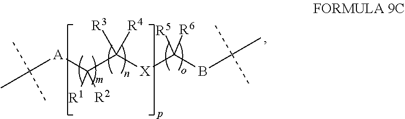

wherein

X is selected from O, NH, and NR⁷;

R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;

A and B are independently selected from null, or bivalent moiety selected from R′—R″, R′COR″, R′CO₂R″, R′C(O)N(R⁸)R″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R′)R″, R′N(R⁸)R″, R′N(R⁸)COR″, R′N(R⁸)C(O)OR″, R′N(R⁸)CON(R⁹)R″, R′N(R⁸)C(S)R″, R′N(R⁸)S(O)R″, R′N(R⁸)S(O)₂R″, R′N(R⁸)S(O)₂N(R⁹)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;

m, at each occurrence, is 0 to 15;

n, at each occurrence, is 0 to 15;

o is 0 to 15; and

p is 0 to 15.

In another refinement, A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH— CH₂—NH—CH₂, CH₂—NH—CH₂.

In another refinement, o is 0 to 5.

In another refinement, the linker moiety comprises a ring selected from the group consisting of a 3 to 13 membered ring, a 3 to 13 membered fused ring, a 3 to 13 membered bridged ring, and a 3 to 13 membered spiro ring.

In another embodiment, the linker moiety comprises one or more rings selected from the group consisting of FORMULAE C1a, C2a, C3a, C4a and C5a

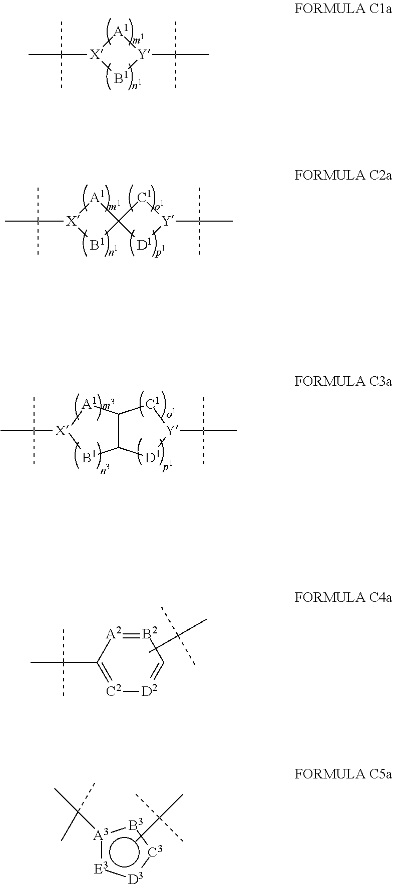

wherein

X′ and Y′ are independently selected from N, CR^{b};

A¹, B¹, C¹ and D¹, at each occurrence, are independently selected from null, O, CO, SO, SO₂, NR^{b}, and CR^{b}R^{c};

A², B², C², and D², at each occurrence, are independently selected from N, and CR^{b};

A³, B³, C³, D³, and E³, at each occurrence, are independently selected from N, O, S, NR^{b}, and CR^{b};

R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and

m¹, n¹, o¹ and p¹ are independently selected from 0, 1, 2, 3, 4 and 5.

In another refinement, the linker moiety comprises one or more rings selected from the group consisting of FORMULAE C1, C2, C3, C4 and C5:

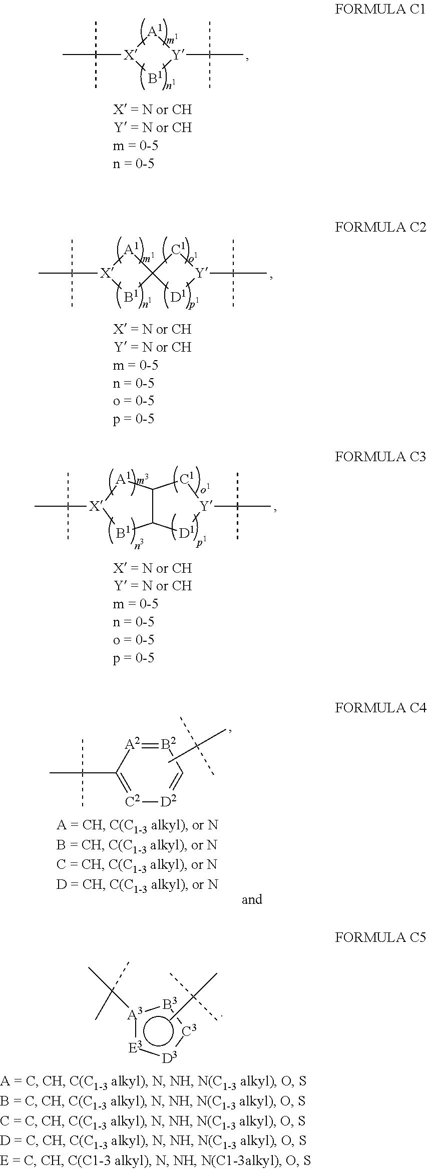

In another refinement, the linker moiety comprises one or more rings selected from Group R, and Group R consists of:

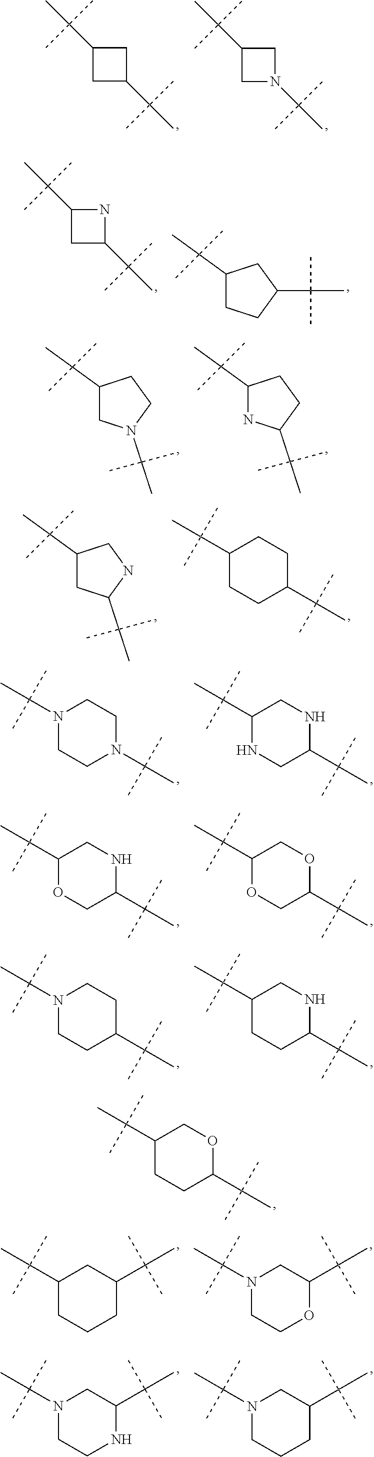

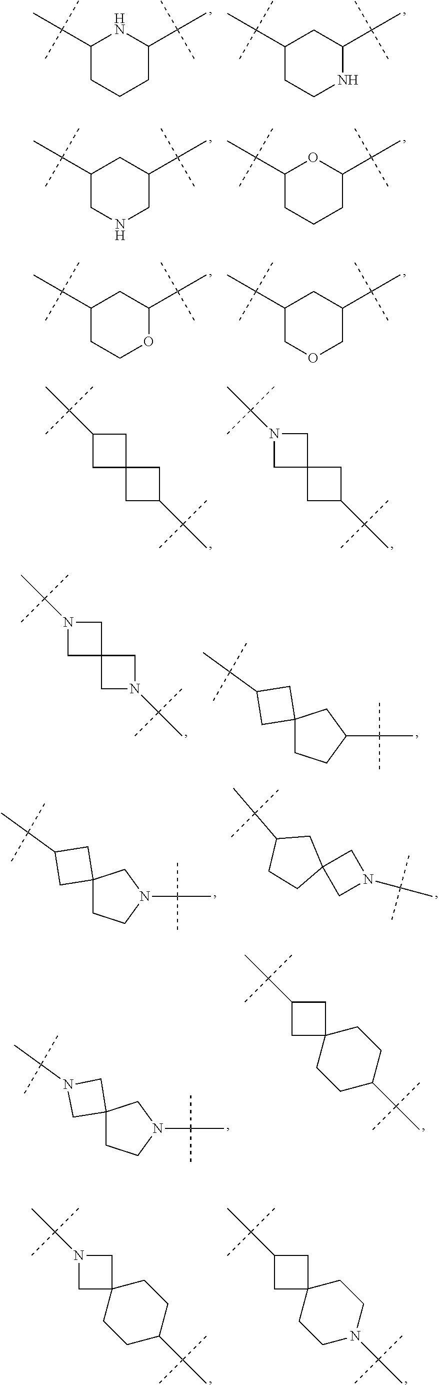

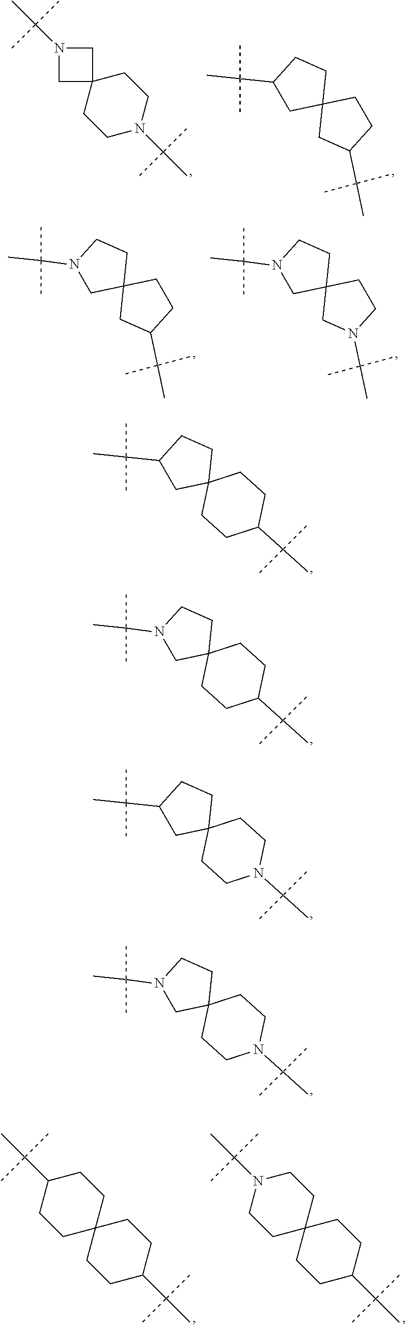

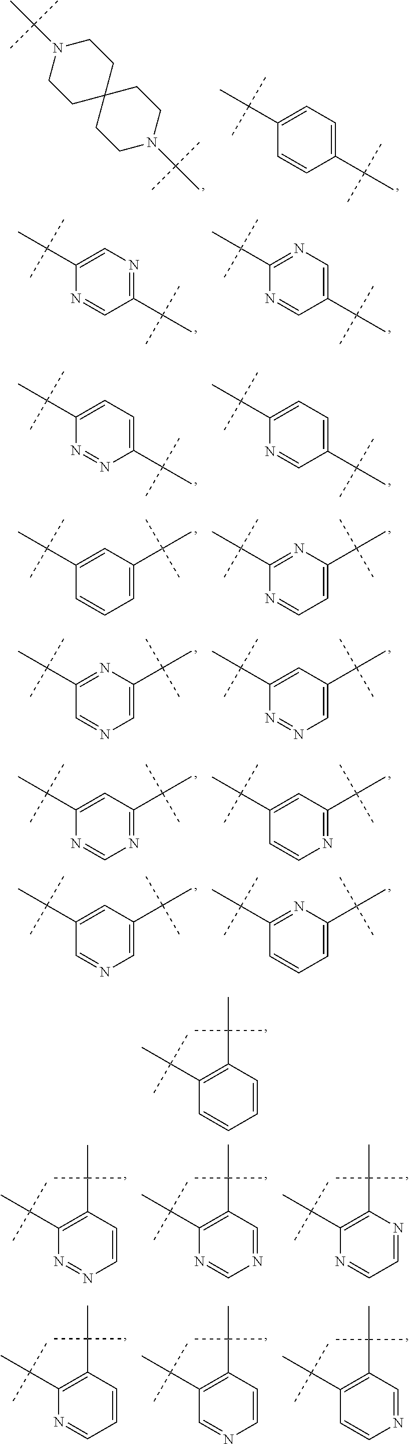

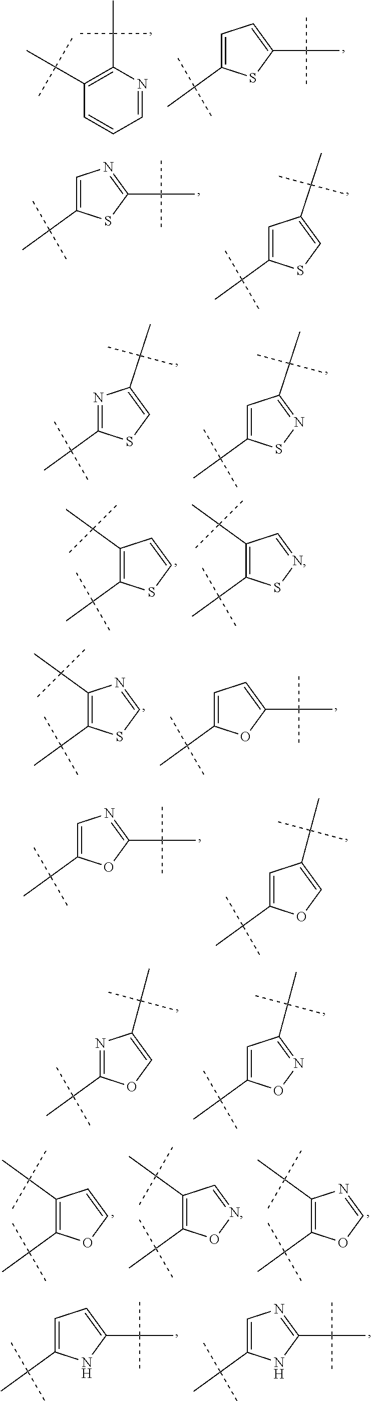

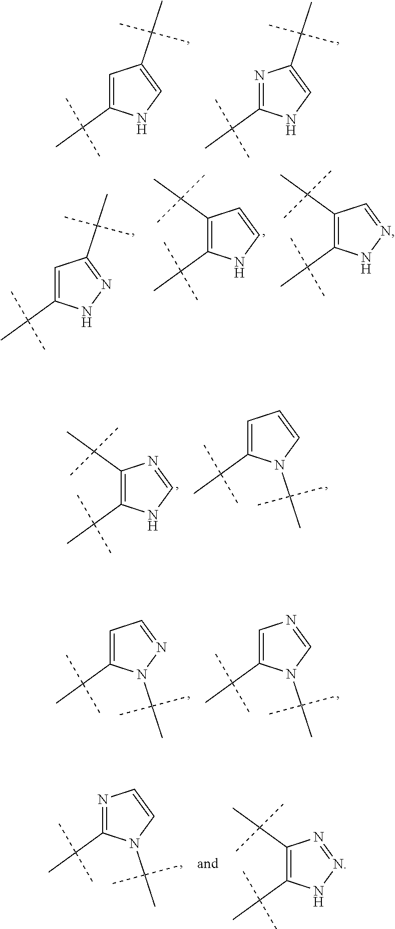

In another refinement, the length of the linker is 0 to 40 chain atoms.

In another refinement, the length of the linker is 1 to 20 chain atoms.

In another refinement, the length of the linker is 2 to 12 chain atoms.

In another refinement, the linker is selected from —(CO)—(CH₂)₁₋₈—, —(CH₂)₁₋₉—, —(CH₂)₁₋₂—(CO)—NH—(CH₂)₂₋₉—, —(CH₂)₁₋₂—(CO)—NH—(CH₂)₁₋₃—(OCH₂CH₂)₁₋₇, and —(CH₂)₀₋₁—(CO)—(CH₂)₁₋₃—(OCH₂CH₂)₁₋₇.

In another refinement, the linker is —(CO)—(CH₂)₁₋₈—, —(CH₂)₁₋₉—, —(CH₂)₁₋₂(CO)—NH—(CH₂)₂₋₉—, or —(CH₂)₁₋₂—(CO)—NH—(CH₂)₁₋₃—(OCH₂CH₂)₁₋₇—.

In another embodiment, R^{r} is selected from FORMULA C1a, C2a, C3a, C4a, C5a, C1, C2, C3, C4, and C5 as defined above.

In another embodiment, R^{r} is selected from Group R.

In some embodiments, the heterobifunctional compound is selected from the group consisting of JA-001 to JA-295 or a pharmaceutically acceptable salt or analog thereof.

In some embodiments, the heterobifunctional compound is selected from the group consisting of JA-093, JA-094, JA-179, JA-180, JA-182, JA-187, JA-188, JA-189, JA-196, JA-198, JA-199, JA-202, JA-203, JA-213, JA-214, JA-224, JA-225, JA-231, JA-252, JA-261, JA-263, JA-264, JA-268, JA-269, JA-273 and a pharmaceutically acceptable salt or analog thereof.

In one embodiment, the heterobifunctional compound is 2-(4-(4-((4-((3-(N-(tert-butyl)sulfamoyl)phenyl)amino)-5-methylpyrimidin-2-yl)amino)phenyl)piperazin-1-yl)-N-(5-((2-(2,6-dioxopiperidin-3-yl)-1,3-dioxoisoindolin-4-yl)amino)pentyl)acetamide (JA-093).

In one embodiment, the heterobifunctional compound is 2-(4-(4-((4-((3-(N-(tert-butyl)sulfamoyl)phenyl)amino)-5-methylpyrimidin-2-yl)amino)phenyl)piperazin-1-yl)-N-(6-((2-(2,6-dioxopiperidin-3-yl)-1,3-dioxoisoindolin-4-yl)amino)hexyl)acetamide (JA-094).

In one embodiment, the heterobifunctional compound is 2-(4-(4-(8-(3,5-difluoro-4-(morpholinomethyl)phenyl)quinoxalin-2-yl)-1H-pyrazol-1-yl)piperidin-1-yl)-N-(7-((2-(2,6-dioxopiperidin-3-yl)-1,3-dioxoisoindolin-5-yl)amino)heptyl)acetamide (JA-179).

In one embodiment, the heterobifunctional compound is 2-(4-(4-(8-(3,5-difluoro-4-(morpholinomethyl)phenyl)quinoxalin-2-yl)-1H-pyrazol-1-yl)piperidin-1-yl)-N-(8-((2-(2,6-dioxopiperidin-3-yl)-1,3-dioxoisoindolin-5-yl)amino)octyl)acetamide (JA-180).

In one embodiment, the heterobifunctional compound is 5-((5-(4-(4-(8-(3,5-difluoro-4-(morpholinomethyl)phenyl)quinoxalin-2-yl)-1H-pyrazol-1-yl)piperidin-1-yl)-5-oxopentyl)amino)-2-(2,6-dioxopiperidin-3-yl)isoindoline-1,3-dione (JA-182).

In one embodiment, the heterobifunctional compound is 5-((8-(4-(4-(8-(3,5-difluoro-4-(morpholinomethyl)phenyl)quinoxalin-2-yl)-1H-pyrazol-1-yl)piperidin-1-yl)-8-oxooctyl)amino)-2-(2,6-dioxopiperidin-3-yl)isoindoline-1,3-dione (JA-187).

In one embodiment, the heterobifunctional compound is 5-((7-(4-(4-(8-(3,5-difluoro-4-(morpholinomethyl)phenyl)quinoxalin-2-yl)-1H-pyrazol-1-yl)piperidin-1-yl)-7-oxoheptyl)amino)-2-(2,6-dioxopiperidin-3-yl)isoindoline-1,3-dione (JA-188).

In one embodiment, the heterobifunctional compound is 5-((6-(4-(4-(8-(3,5-difluoro-4-(morpholinomethyl)phenyl)quinoxalin-2-yl)-1H-pyrazol-1-yl)piperidin-1-yl)-6-oxohexyl)amino)-2-(2,6-dioxopiperidin-3-yl)isoindoline-1,3-dione (JA-189).

In one embodiment, the heterobifunctional compound is 2-(2,6-dioxopiperidin-3-yl)-5-((5-(4-(4-((5-(4-(methylsulfonyl)phenyl)-[1,2,4]triazolo[1,5-a]pyridin-2-yl)amino)phenyl)piperazin-1-yl)-5-oxopentyl)amino)isoindoline-1,3-dione (JA-196).

In one embodiment, the heterobifunctional compound is 2-(2,6-dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)isoindoline-1,3-dione (JA-198).

In one embodiment, the heterobifunctional compound is 2-(2,6-dioxopiperidin-3-yl)-5-((7-(4-(4-((5-(4-(methylsulfonyl)phenyl)-[1,2,4]triazolo[1,5-a]pyridin-2-yl)amino)phenyl)piperazin-1-yl)-7-oxoheptyl)amino)isoindoline-1,3-dione (JA-199).

In one embodiment, the heterobifunctional compound is 2-(2,6-dioxopiperidin-3-yl)-5-((3-(4-(4-((5-(4-(methylsulfonyl)phenyl)-[1,2,4]triazolo[1,5-a]pyridin-2-yl)amino)phenyl)piperidin-1-yl)-3-oxopropyl)amino)isoindoline-1,3-dione (JA-202).

In one embodiment, the heterobifunctional compound is 2-(2,6-dioxopiperidin-3-yl)-5-((8-(4-(4-((5-(4-(methylsulfonyl)phenyl)-[1,2,4]triazolo[1,5-a]pyridin-2-yl)amino)phenyl)piperidin-1-yl)-8-oxooctyl)amino)isoindoline-1,3-dione (JA-203).

In one embodiment, the heterobifunctional compound is N-(8-((2-(2,6-dioxopiperidin-3-yl)-1,3-dioxoisoindolin-5-yl)amino)octyl)-2-(4-(4-((5-(4-(methylsulfonyl)phenyl)-[1,2,4]triazolo[1,5-a]pyridin-2-yl)amino)phenyl)piperidin-1-yl)acetamide (JA-213).

In one embodiment, the heterobifunctional compound is N-(7-((2-(2,6-dioxopiperidin-3-yl)-1,3-dioxoisoindolin-5-yl)amino)heptyl)-2-(4-(4-((5-(4-(methylsulfonyl)phenyl)-[1,2,4]triazolo[1,5-a]pyridin-2-yl)amino)phenyl)piperidin-1-yl)acetamide (JA-214).

In one embodiment, the heterobifunctional compound is 2-(4-(4-((4-((3-(N-(tert-butyl)sulfamoyl)phenyl)amino)-5-methylpyrimidin-2-yl)amino)phenyl)piperazin-1-yl)-N-(17-((2-(2,6-dioxopiperidin-3-yl)-1,3-dioxoisoindolin-4-yl)amino)-3,6,9,12,15-pentaoxaheptadecyl)acetamide (JA-224).

In one embodiment, the heterobifunctional compound is N-(tert-butyl)-3-((2-((4-(4-((2-(2,6-dioxopiperidin-3-yl)-1,3-dioxoisoindolin-5-yl)glycyl)piperazin-1-yl)phenyl)amino)-5-methylpyrimidin-4-yl)amino)benzenesulfonamide (JA-225).

In one embodiment, the heterobifunctional compound is N-(tert-butyl)-3-((2-((4-(4-(8-((2-(2,6-dioxopiperidin-3-yl)-1,3-dioxoisoindolin-5-yl)amino)octanoyl)piperazin-1-yl)phenyl)amino)-5-methylpyrimidin-4-yl)amino)benzenesulfonamide (JA-231).

In one embodiment, the heterobifunctional compound is 2-(2,6-dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)hept-1-yn-1-yl)isoindoline-1,3-dione (JA-252).

In one embodiment, the heterobifunctional compound is 3-(5-((6-(4-(4-((5-(4-(methylsulfonyl)phenyl)-[1,2,4]triazolo[1,5-a]pyridin-2-yl)amino)phenyl)piperazin-1-yl)-6-oxohexyl)oxy)-1-oxoisoindolin-2-yl)piperidine-2,6-dione (JA-261).

In one embodiment, the heterobifunctional compound is 3-(5-((6-(4-(4-(8-(3,5-difluoro-4-(morpholinomethyl)phenyl)quinoxalin-2-yl)-1H-pyrazol-1-yl)piperidin-1-yl)-6-oxohexyl)oxy)-1-oxoisoindolin-2-yl)piperidine-2,6-dione (JA-263).

In one embodiment, the heterobifunctional compound is 5-(7-(4-(4-(8-(3,5-difluoro-4-(morpholinomethyl)phenyl)quinoxalin-2-yl)-1H-pyrazol-1-yl)piperidin-1-yl)hept-1-yn-1-yl)-2-(2,6-dioxopiperidin-3-yl)isoindoline-1,3-dione (JA-264).

In one embodiment, the heterobifunctional compound is 3-(6-(7-(4-(4-((5-(4-(methylsulfonyl)phenyl)-[1,2,4]triazolo[1,5-a]pyridin-2-yl)amino)phenyl)piperazin-1-yl)-7-oxoheptyl)-1-oxoisoindolin-2-yl)piperidine-2,6-dione (JA-268).

In one embodiment, the heterobifunctional compound is 3-(6-(7-(4-(4-(8-(3,5-difluoro-4-(morpholinomethyl)phenyl)quinoxalin-2-yl)-1H-pyrazol-1-yl)piperidin-1-yl)-7-oxoheptyl)-1-oxoisoindolin-2-yl)piperidine-2,6-dione (JA-269).

In one embodiment, the heterobifunctional compound is 3-(5-((6-(4-(4-(8-(3,5-difluoro-4-(morpholinomethyl)phenyl)quinoxalin-2-yl)-1H-pyrazol-1-yl)piperidin-1-yl)-6-oxohexyl)amino)-1-oxoisoindolin-2-yl)piperidine-2,6-dione (JA-273),

According to one aspect of the present disclosure, a composition disclosed herein comprises the heterobifunctional compound or a pharmaceutically acceptable salt or analog thereof.

According to one aspect of the present disclosure, a method of treating a JAK-mediated disease disclosed herein comprises administering to a subject with a JAK-mediated disease the heterobifunctional compound or a pharmaceutically acceptable salt or analog thereof.

In one embodiment, the JAK-mediated disease results from JAK expression, mutation, deletion, or fusion.

In one embodiment, the subject with the JAK-mediated disease has an elevated JAK function relative to a healthy subject without the JAK-mediated disease.

In one embodiment, the heterobifunctional compound is selected from the group consisting of JA-001 to JA-295, or analogs thereof.

In one embodiment, the heterobifunctional compound is administered to the subject orally, parenterally, intradermally, subcutaneously, topically, or rectally.

In one embodiment, the method further comprises administering to the subject an additional therapeutic regimen for treating cancer, inflammatory disorders, or autoimmune diseases.

In one embodiment, the additional therapeutic regimen is selected from the group consisting of surgery, chemotherapy, radiation therapy, hormone therapy, targeted therapy, and immunotherapy.

In one embodiment, the JAK-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, and myeloma.

In one embodiment, the JAK-mediated inflammatory disorders are selected from the group consisting of ankylosing spondylitis, Crohn's disease, inflammatory bowel disease, ulcerative colitis, and ischemia reperfusion injuries.

In one embodiment, the JAK-mediated auto-immune diseases are selected from the group consisting of multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid.

In one embodiment, the JAK-mediated dermatological disorders are selected from the group consisting of atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING vasculopathy.

In one embodiment, the JAK-mediated viral infections are selected from the group consisting of infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV).

In one embodiment, the JAK-mediated dry eye disorders are selected from the group consisting of dry eye syndrome (DES) and keratoconjunctivitis sicca (KCS).

In one embodiment, the JAK-mediated bone remodeling disorders are selected from the group consisting of osteoporosis and osteoarthritis.

In one embodiment, the JAK-mediated organ transplant associated immunological complications are selected from the group consisting of graft-versus-host diseases.

In one embodiment, the JAK-mediated disease is a relapsed cancer.

In one embodiment, the JAK-mediated disease is refractory to one or more previous treatments.

According to one aspect of the present disclosure, a method for identifying a heterobifunctional compound which mediates degradation or reduction of JAK is disclosed. The method comprises:

providing a heterobifunctional test compound comprising an JAK ligand conjugated to a degradation tag through a linker;

contacting the heterobifunctional test compound with a cell comprising a ubiquitin ligase and JAK;

determining whether JAK level is decreased in the cell; and

identifying the heterobifunctional test compound as a heterobifunctional compound which mediates degradation or reduction of JAK.

In one embodiment, the cell is a cancer cell.

In one embodiment, the cancer cell is a JAK-mediated cancer cell.

According to one aspect of the present disclosure, a method of treating a GSTP1-mediated disease disclosed herein comprises administering to a subject with a GSTP1-mediated disease the heterobifunctional compound or a pharmaceutically acceptable salt or analog thereof.

According to one aspect of the present disclosure, a method of treating a JAK- and GSTP1-mediated disease disclosed herein comprises administering to a subject with a JAK- and GSTP1-mediated disease the heterobifunctional compound or a pharmaceutically acceptable salt or analog thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an immunoblot of JAK1/2/3 proteins expressed in HEL cells after treatment with a dose range of heterobifunctional compounds JA-189 or JA-213 or a single dose of NVP-BSK805.

FIG. 2 shows an immunoblot of JAK1/3 proteins expressed in RS4;11 cells after treatment with a dose range of compounds JA-189, JA-213, NVP-BSK805 or TG101209.

FIG. 3 shows graphs of MV4;11, RS4;11, Kasumi-1 and HEL cell viability vs. concentration of JA-189, JA-213, NVP-BSK805 and TG101209.

FIG. 4 shows graphs of MV4;11, RS4;11, Kasumi-1 and HEL cell viability vs. concentration of JA-189, JA-213, NVP-BSK805 and TG101209 in the presence or absence of pomalidomide at 10 μM.

FIG. 5 shows an immunoblot of GSPT1 and tubulin expressed in RS4:11 cells treated with JA-189, JA-213, or CC-885 at indicated concentrations for 16 hours.

FIG. 6 shows graphs of immortalized human lung fibroblast IMR-90 and keratinocyte HACAT cell viability vs. concentration of JA-189, and JA-213.

### DETAILED DESCRIPTION OF THE INVENTION

The human Janus kinase (JAK) family comprises four nonreceptor tyrosine kinases, JAK1, JAK2, JAK3 and TYK2. JAK kinases play a central role in the hematopoietic system through transducing cytokine-mediated signals (O'Shea, Schwartz et al. 2015). These intracellular kinases bound to type I and II cytokine receptors, which lack catalytic domains and reply on the kinase activities of JAKs for transducing signals to downstream molecules. Cytokine binding triggers conformational changes of their respective receptors and subsequently induces autophosphorylation and activation of JAK kinases bound to the receptors. JAK kinases then recruit and phosphorylate downstream signaling molecules, most importantly the signal transducer and activator of transcription (STAT) family transcription factors. Phosphorylated STATs are subsequently translocated into nucleus and activate transcription of genes implicated in hematopoiesis and immune response. Different cytokine receptors recruit distinct combinations of JAK kinases, which transduce signals to a wide range of downstream targets, modulating complex and lineage-dependent signaling networks (Schwartz, Kanno et al. 2017).

Acting downstream of a variety of cytokine receptors, JAK kinases are crucially implicated in proliferation, survival, activation, and differentiation of hematopoietic cells (Villarino, Kanno et al. 2015). Furthermore, there is no known pathways that may adequately compensate the JAK/STAT signaling in the hematopoietic system. Hence, the activities of JAK kinases are essential to hematopoiesis and immunity. As a consequence, aberrations of JAK kinases are known to drive the pathogenesis of many diseases, most significantly inflammation, autoimmune diseases, and cancer (O'Shea, Schwartz et al. 2015). For many of these indications, JAK family kinases are well documented as important therapeutic targets.

Mutations leading to constitutive JAK2 activation are found in the majority of BCR-ABL-negative myeloproliferative neoplasms (MPNs), including essential thrombocythemia (ET), polycythemia vera (PV), and primary myelofibrosis (PMF) (Levine, Wadleigh et al. 2005, Griesshammer and Sadjadian 2017). Within these indications, JAK2-V617F is the most common mutation. JAK2 mutations have also been described in chronic neutrophilic leukemia, acute lymphoblastic leukemia, Hodgkin's lymphoma, and other hematologic malignancies. In addition to JAK2 point mutations, fusions of JAK2, and mutations of JAK1 and JAK3 have also been identified as mechanisms activating the JAK pathway, albeit to less extent (O'Shea, Holland et al. 2013). Alternative mechanisms to activate JAK kinases include mutations of JAK regulators, such as CSFR3 (Maxson, Gotlib et al. 2013), MPL (Kilpivaara and Levine 2008), and CALR (Rumi, Pietra et al. 2014). Activation of JAK2 is also implicated in non-malignant hematopoietic indications, such as hereditary thrombocythemia (Langabeer 2014). Importantly, a wide range of immunological disorders involves aberrant activation of JAK kinases, such as rheumatoid arthritis, atopic dermatitis, psoriasis, pruritus, inflammatory bowel disease, Crohn's disease, ulcerative colitis, psoriatic arthritis, juvenile idiopathic arthritis, ankylosing spondylitis, alopecia areata, systemic lupus erythematosus, and graft-versus-host disease (Schwartz, Kanno et al. 2017).

Three JAK inhibitors have been approved for use in human for treatment of rheumatoid arthritis including Ruxolitinib (Mesa, et al., 2012), Tofacitinib (CP-690550) (Traynor, 2012; Lee, et al., 2014; Dhillon, 2017; Strand, et al., 2019), and Baricitinib (Taylor, et al., 2017; Markham, 2017), and the JAK inhibitor Oclacitinib (Gonzales, et al., 2014) has been approved for treatment of canine allergic dermatitis. Multiple JAK kinase inhibitors are currently undergoing clinical or pre-clinical development, including but are not limited to Cerdulatinib (PRT-062070) (Hamlin, et al., 2019), Decemotinib (VX509) (Farmer, et al., 2015), Delgocitinib (JTE-052) (Nakagawa, et al., 2018), Fedratinib (Wernig, et al., 2008; Harrison, et al., 2017), Filgotinib (GLP0634) (Menet, et al., 2014; Van Rompaey, et al., 2013), Gandotinib (LY2784544) (Berdeja, et al., 2018), Ilginatinib (NS-018) (Nakaya, et al., 2011; Verstovsek, et al., 2016), Itacitinib (INCB03911) (Beatty, et al., 2018), Lestauritinib (Mascarenhas, et al., 2019; Pinto, et al., 2018), Momelotinib (CYT387) (Pardanani, et al., 2018), Pacritinib (SB1578) (Tremblay, et al., 2018; William, et al., 2012), Peficitinib (Takeuchi, et al., 2016; Hamaguchi, et al., 2018), Solcitinib (GSK2586184, GLG0778) (Kahl, et al., 2016), Upadacitinib (ABT-494) (Serhal, et al., 2018; Genovese, et al., 2018), AT9283 (Howard, et al., 2009), AZ-3 (Grimster, et al., 2018), AZ960 (Gozgit, et al., 2008), AZD1480 (Verstovsek, et al., 2015; Ioannidis, et al., 2011), BMS-986165 (Papp, et al., 2018), BMS-911543 (Wan, et al., 2015), BSK805 (NVP-BSK805) and BVB808 (NVP-BVB808) (Ringel, et al., 2014; Andraos, et al., 2012), BBT594 (NVP-BBT594) (Koppikar, et al., 2012; Andraos, et al., 2012), CEP-33799 (Dugan, et al., 2012), CHZ868 (Wu, et al., 2015), FM381 (Forster, et al., 2016; Forster, et al., 2018), PF-04965842 (Vazquez, et al., 2018), PF-06263276 (Jones, et al., 2017), PF-06651600 (Thorarensen, et al., 2017; Telliez, et al., 2016), PF-06700841 (Fensome, et al., 2018), SAR-20347 (Works, et al., 2014), NDI-031301 (Akahane, et al., 2017), NDI-31232 (Masse, et al., 2015), NVP-P830 (Brasca, et al., 2015) SHR-0302 (Wu, et al., 2016), VR588 (Wiegman, et al., 2015), XL019 (Forsyth, et al., 2012), TG101209 (Demyanets, et al., 2018; Pardanani, et al., 2007), R333 and R348 (Deuse, et al., 2008), 3-amido pyrrolopyrazine (Cmpd 3q) (Soth, et al., 2013), pyridone containing tetracycle (Cmpd 6) (Thompson, et al., 2001; Williams, et al., 2009), triazolo-pyrrolopyridines (Cmpd 7) (Hurley, et al., 2013), pyrazolopyrimidines (Cmpd 7j) (Hanan, et al., 2012), 1-amino-[1,2,4]triazolo[1,5-a]pyridines (Cmpd 12) (Siu, et al., 2013), imidazolopyridines (Cmpd 19) (Simov, et al., 2016), 1-methyl-1H-imidazole derivatives (Cmpd 19a) (Su, et al., 2014), C-2 methyl imidazopyrrolopyridines (Cmpd 20) (Zak, et al., 2012), pyrazolopyridinone (Cmpd 20a) (Yogo, et al., 2016), 9H-carbazole-1-carboxamides (Cmpd 21) (Zimmermann, et al., 2015), thianopyridines (Cmpd 23) (Schenkel, et al., 2011), pyrazole-4-carboxamide (Cmpd 28) (Siu, et al., 2017), imidazopyridine (Cmpd 30) (Liang, et al., 2017), hydroxyethyl imidazo-pyrrolopyridines (Cmpd 31) (Zak, et al., 2013), pyrrolopyridazines (Cmpd 35) (Hynes, et al., 2017), 6-oxopyridopyrimidines (Compound 36) (Labadie, et al., 2013), 2-aminopyrazolo[1,5-a]pyrimidines (Cmpd 45) (Ledeboer, et al., 2009), cyclopropyl amides (Cmpd 46) (Liang, et al., 2013; Liang, et al., 2013), imidazo-pyrrolopyridines (Cmpd 49) (Kulagowski, et al., 2012), 1-amino-5H-pyrido[4,3-b]indol-4-carboxamides (Cmpd 65) (Lim, et al., 2011), and covalent selective inhibitors of JAK3 (Cmpd 3) (Goedken, et al., 2015), (Cmpd 13a) (Kempson, et al., 2017), and (Cmpd 45a) (Tan, et al., 2015).

Despite the approval of JAK2 kinase inhibitors for the treatment of MPNs, the efficacy of these drugs is generally modest and short-lived (Tefferi 2012). A key mechanism of resistance to JAK2 kinase inhibitors is the kinase-independent functions of JAK2. Knockout of JAK2 in mouse models results in embryonic lethality due to a complete loss of EpoR signaling and a lack of erythropoiesis (Neubauer, Cumano et al. 1998). JAK2-knockout mice also show deficient interferon γ signaling. The tyrosine residues 1007/1008 of JAK2 within the kinase activation loop are essential for its kinase activity. Using a genetically engineered mouse model that expresses a kinase-dead mutant (YY1007/1008FF) of JAK2, Keil and colleagues demonstrate that kinase-dead JAK2 partially sustains the interferon γ signaling, possibly through acting as a scaffolding protein at the heteromeric interferon γ receptor (Keil, Finkenstadt et al. 2014). It is also well documented that JAK2 kinase inhibitors stimulate the activation loop phosphorylation, leading to reactivation of JAK signaling through dimerization of JAK2 with other JAK kinases, and consequently promoting resistance to JAK2 kinase inhibitors in MPNs (Koppikar, Bhagwat et al. 2012). Most importantly, cells resistant to JAK2 kinase inhibitors remain sensitive to depletion of JAK2 expressions (Koppikar, Bhagwat et al. 2012). Therefore, depleting JAK2 protein is an appealing strategy to improve outcomes of patients with JAK2-driven MPNs.

Without wishing to be bound by any theory, the present disclosure is believed to be based, at least in part, on the discovery that novel heterobifunctional small molecules which degrade JAK (e.g. JAK1, JAK2, JAK3, and TYK2), JAK fusion proteins, JAK deletion proteins, and/or JAK mutant proteins are useful in the treatment of JAK-mediated diseases: such as cancer (e.g. cancers of brain, stomach, gastrointestinal tracts, liver, biliary passage, breast, ovary, cervix, prostate, testis, penile, genitourinary tract, esophagus, larynx, skin, lung, pancreas, thyroid, glands, bladder, kidney, muscle, bone, and cancers of the hematopoietic system, such as myeloproliferative neoplasms, including essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, myeloma, and other hematologic malignancies, particularly cancers that involve inflammation, mutations or other aberrations that activate the JAK pathway) (LaFave and Levine 2012, O'Shea, Holland et al. 2013); inflammation (e.g. ankylosing spondylitis, Crohn's disease, inflammatory bowel disease, ulcerative colitis, and ischemia reperfusion injuries, which are conditions related to inflammatory ischemic events such as stroke or cardiac arrest) (Schwartz, Kanno et al. 2017); auto-immune diseases (e.g. multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid) (O'Shea, Kontzias et al. 2013); dermatological disorders (e.g. atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING-associated vasculopathy) (Damsky and King 2017); viral infections (e.g. viral infections and consequent complications, such as infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV)) (Fleming 2016); dry eye disorder, also known as dry eye syndrome (DES) or keratoconjunctivitis sicca (KCS) (Colligris, Alkozi et al. 2014); bone remodeling disorders (e.g. osteoporosis and osteoarthritis) (Li 2013); organ transplant associated immunological complications (e.g. graft-versus-host diseases) (Moore, Iasella et al. 2017).

Termination of translation is a GTP-dependent process that is regulated by two key proteins eRF1 and eRF3. The translation termination factor eRF3a (also known as GSPT1) is a GTPase that interacts with eRF1 to promote stop codon recognition and release of nascent peptide from ribosome (Chauvin, Salhi et al. 2005). GSPT1 activates eRF1 in a GTP-dependent manner and its GTPase activity requires complexing with eRF1 and ribosomes (Frolova, Le Goff et al. 1996). The GTP-bound GSPT1 and eRF1 together with ribosomes form the functional translation termination complexes (Zhouravleva, Frolova et al. 1995). Through regulation of translation, GSPT1 has diverse and important roles in cell physiology. Increased expression of GSPT1 has been reported in human malignancies, including lung cancer and gastric cancer (Malta-Vacas, Aires et al. 2005, Tian, Tian et al. 2018, Sun, Zhang et al. 2019, Zhang, Zou et al. 2019). Hence, GSPT1 is thought to be a novel cancer target through which may compromise active translation that contributes to malignant phenotypes of cancer cells. Recently, Matyskiela and colleagues have reported that a phthalimide-derived molecule CC-885 led to cereblon-dependent degradation of GSPT1 and other targets, such as IKZF1 and IKZF3 (Matyskiela, Lu et al. 2016). Ishoey et al. also reported that GSPT1 was degraded by a subset of heterobiofunctional compounds derived from phthalimide (Ishoey, Chorn et al. 2018). CC-885 induced significant toxicity in the vast majority of tested cell lines, presumably due to degrading GSTP1 and many other proteins (Matyskiela, Lu et al. 2016). Therefore, despite the broad and potent anti-cancer activity, CC-885 exhibits unacceptable toxicity that prevents further development (Hansen, Correa et al. 2020).

Selective degradation of a target protein induced by a small molecule may be achieved by recruiting an E3 ubiquitin ligase and mimicking protein misfolding with a hydrophobic tag (Buckley and Crews 2014). Additionally, protein degraders are heterobifunctional compounds having one moiety that binds to an E3 ubiquitin ligase and another moiety that binds the protein target of interest (Buckley and Crews 2014). The induced proximity leads to ubiquitination of the target followed by its degradation via proteasome-mediated proteolysis. Several types of high affinity small-molecule E3 ligase ligands have been identified or developed. They include (1) immunomodulatory drugs (IMiDs) such as thalidomide and pomalidomide, which bind cereblon (CRBN or CRL4CRBN), a component of a cullin-RING ubiquitin ligase (CRL) complex (Ito, Ando et al. 2010, Chamberlain, Lopez-Girona et al. 2014, Fischer, Bohm et al. 2014, Bondeson, Mares et al. 2015, Winter, Buckley et al. 2015); (2) VHL-1, a hydroxyproline-containing ligand, which binds van Hippel-Lindau protein (VHL or CRL2VHL), a component of another CRL complex (Buckley, Gustafson et al. 2012, Buckley, Van Molle et al. 2012, Galdeano, Gadd et al. 2014, Bondeson, Mares et al. 2015, Zengerle, Chan et al. 2015); (3) compound 7, which selectively binds KEAP1, a component of a CRL3 complex (Davies, Wixted et al. 2016); (4) AMG232, which selectively binds MDM2, a heterodimeric RING E3 ligase (Sun, Li et al. 2014); and (5) LCL161, which selectively binds IAP, a homodimeric RING E3 ligase (Okuhira, Ohoka et al. 2011, Ohoka, Okuhira et al. 2017, Shibata, Miyamoto et al. 2017). The PROTAC technology has been applied to degradation of several protein targets (Bondeson, Mares et al. 2015, Buckley, Raina et al. 2015, Lu, Qian et al. 2015, Winter, Buckley et al. 2015, Zengerle, Chan et al. 2015, Lai, Toure et al. 2016). In addition, a hydrophobic tagging approach, which utilizes a bulky and hydrophobic adamantyl group, has been developed to mimic protein misfolding, leading to the degradation of the target protein (Buckley and Crews 2014). This approach has been applied to selective degradation of the pseudokinase HER3 (Xie, Lim et al. 2014). The inventors have not yet seen any efforts applying any of these approaches to degradation of JAK (e.g. JAK1, JAK2, JAK3, and TYK2), JAK mutant, JAK deletion, or JAK fusion proteins.

Currently available small molecules targeting JAK (e.g. JAK1, JAK2, JAK3, and TYK2) focus on inhibition of JAK kinase activities.

In the present disclosure, a novel approach is taken: to develop compounds that directly and selectively modulate not only the kianse activity of JAK (e.g. JAK1, JAK2, JAK3, and TYK2), but also their protein level. Strategies for inducing protein degradation include recruiting E3 ubiquitin ligases, mimicking protein misfolding with hydrophobic tags, and inhibiting chaperones. Such an approach, based on the use of heterobifunctional small molecule compounds, permits more flexible regulation of protein levels in vitro and in vivo compared with techniques such as genetic knockout or knockdown. Unlike genetic knockout or knockdown, a small molecule approach further provides an opportunity to study dose and time dependency in a disease model through modulating the administration routes, concentrations and frequencies of administration of the corresponding small molecule.

This disclosure includes all stereoisomers, geometric isomers, tautomers and isotopes of the structures depicted and compounds named herein. This disclosure also includes compounds described herein, regardless of how they are prepared, e.g., synthetically, through biological process (e.g., metabolism or enzyme conversion), or a combination thereof.

This disclosure includes pharmaceutically acceptable salts of the structures depicted and compounds named herein.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms. In some embodiments, the compound includes at least one fluorine atom. In some embodiments, the compound includes two or more fluorine atoms. In some embodiments, the compound includes 1-2, 1-3, 1-4, 1-5, or 1-6 fluorine atoms. In some embodiments, all of the hydrogen atoms in a compound can be replaced or substituted by fluorine atoms.

Heterobifunctional Compounds

As used herein, the term “heterobifunctional compound(s)” and “bivalent compound(s)” can be used interchangeably.

In some aspects, the present disclosure provides heterobifunctional compounds including a JAK ligand conjugated to a degradation tag, or a pharmaceutically acceptable salt or analog thereof. The JAK ligand may be conjugated to the degradation tag directly or via a linker moiety. In certain embodiments, the JAK ligand may be conjugated to the degradation tag directly. In certain embodiments, the JAK ligand may be conjugated to the degradation tag via a linker moiety.

As used herein, the terms “Janus kinase ligand” and “JAK ligand”, or “JAK targeting moiety” are to be construed to encompass any molecules ranging from small molecules to large proteins that associate with or bind to any of JAK1, JAK2, JAK3, or TYK2 proteins. In certain embodiments, the JAK ligand is capable of binding to a JAK protein comprising JAK (e.g. JAK1, JAK2, JAK3, and TYK2), a JAK mutant, a JAK deletion, or a JAK fusion protein. The JAK ligand can be, for example but not limited to, a small molecule compound (i.e., a molecule of molecular weight less than about 1.5 kilodaltons (kDa)), a peptide or polypeptide, nucleic acid or oligonucleotide, carbohydrate such as oligosaccharides, or an antibody or fragment thereof.

JAK Ligand

The JAK ligand or targeting moiety can be a JAK inhibitor or a portion of JAK inhibitor. In certain embodiments, the JAK inhibitor comprises one or more of (e.g., Ruxolitinib, Tofacitinib (CP-690550), Baricitinib, Oclacitinib, Cerdulatinib (PRT-062070), Decernotinib (VX509), Delgocitinib (JTE-052), Fedratinib, Filgotinib (GLP0634), Gandotinib (LY2784544), Ilginatinib (NS-018), Itacitinib (INCB03911), Lestauritinib, Momelotinib (CYT387), Pacritinib (SB1578), Peficitinib, Solcitinib (GSK2586184, GLG0778), Upadacitinib (ABT-494), AT9283, AZ-3, AZ960, AZD1480, BMS-986165, BMS-911543, BSK805 (NVP-BSK805) and BVB808 (NVP-BVB808), BBT594 (NVP-BBT594), CEP-33799, CHZ868, FM381, PF-04965842, PF-06263276, PF-06651600, PF-06700841, SAR-20347, NDI-031301 (Akahane, Li, & Etchin, 2017), NDI-31232 (Masse, Miao, Greenwood, Shelley, & Kapeller, 2015), NVP-P830, SHR-0302, VR588 (Wiegman, Adcock, Rothaul, Main, & Morgan, 2015), XL019, TG101209, R333 and R348 (Deuse, et al., 2008), 3-amido pyrrolopyrazine (Cmpd 3q), pyridone containing tetracycle (Cmpd 6), triazolo-pyrrolopyridines (Cmpd 7), pyrazolopyrimidines (Cmpd 7j), 1-amino-[1,2,4]triazolo[1,5-a]pyridines (Cmpd 12), imidazolopyridines (Cmpd 19), 1-methyl-1H-imidazole derivatives (Cmpd 19a), C-2 methyl imidazopyrrolopyridines (Cmpd 20), pyrazolopyridinone (Cmpd 20a), 9H-carbazole-1-carboxamides (Cmpd 21), thianopyridines (Cmpd 23), pyrazole-4-carboxamide (Cmpd 28), imidazopyridine (Cmpd 30), hydroxyethyl imidazo-pyrrolopyridines (Cmpd 31), pyrrolopyridazines (Cmpd 35), 6-oxopyridopyrimidines (Compound 36), 2-aminopyrazolo[1,5-a]pyrimidines (Cmpd 45), cyclopropyl amides (Cmpd 46), imidazo-pyrrolopyridines (Cmpd 49), 1-amino-5H-pyrido[4,3-b]indol-4-carboxamides (Cmpd 65), Cmpd 3, Cmpd 13a, Cmpd 45a, and analogs thereof), which is capable of inhibiting the protein-protein interaction or acetyltransferase activity of JAK. As used herein, a “JAK inhibitor” refers to an agent that restrains, retards, or otherwise causes inhibition of a physiological, chemical or enzymatic action or function and causes a decrease in binding of at least 5%. An inhibitor can also or alternately refer to a drug, compound, or agent that prevents or reduces the expression, transcription, or translation of a gene or protein. An inhibitor can reduce or prevent the function of a protein, e.g., by binding to or activating/inactivating another protein or receptor.

In certain embodiments, the JAK ligand is derived from a JAK inhibitor comprising:

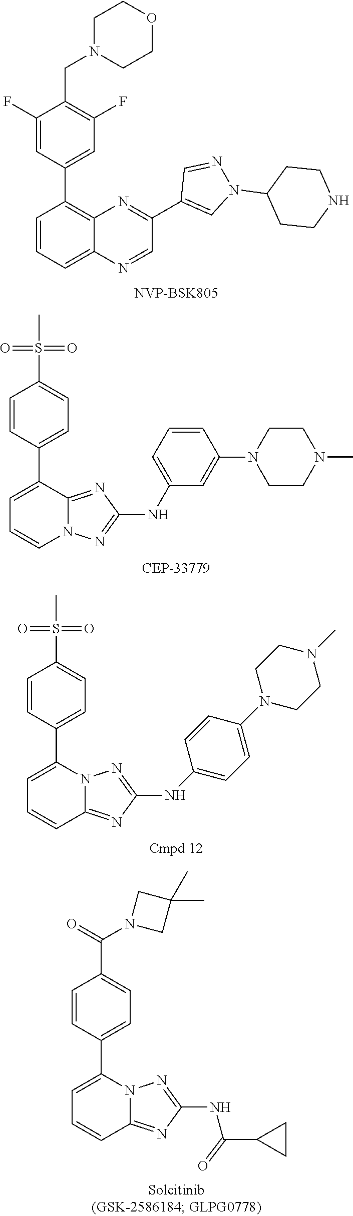

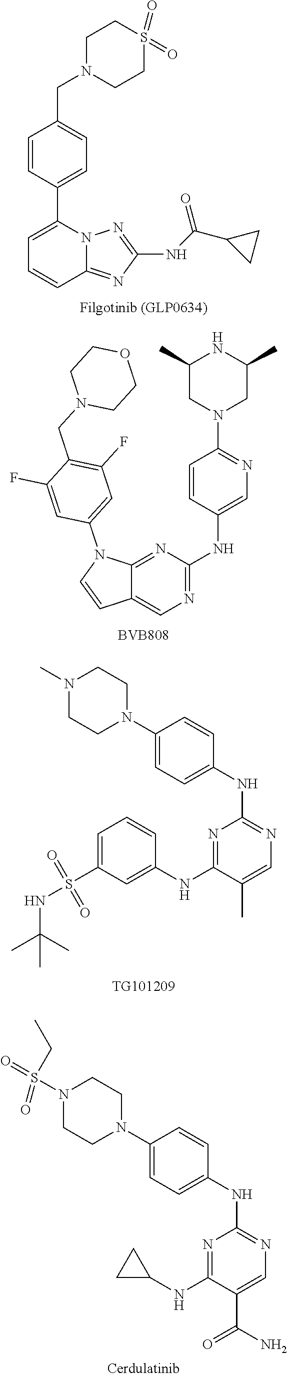

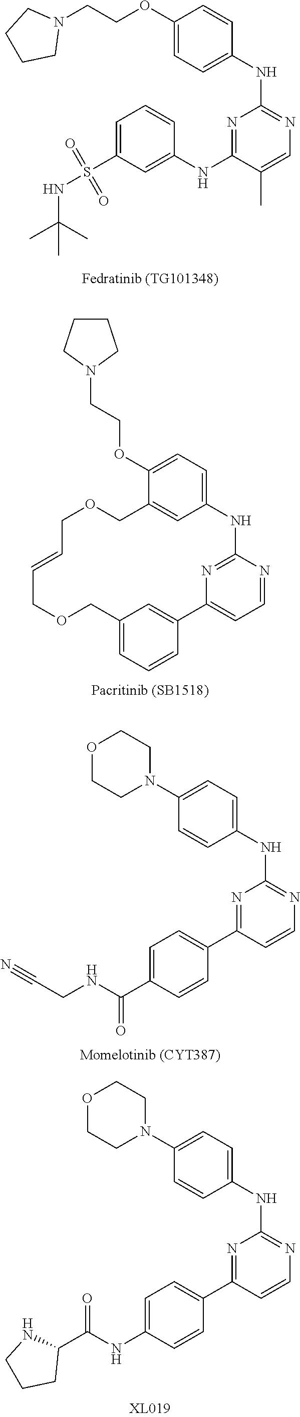 [SMILES: Cc1cnc(Nc2ccc(OCCN3CCCC3)cc2)nc1Nc1cccc(S(=O)(=O)NC(C)(C)C)c1 ; C1=C/COCc2cc(ccc2OCCN2CCCC2)Nc2nccc(n2)-c2cccc(c2)COC/1 ; N#CCNC(=O)c1ccc(-c2ccnc(Nc3ccc(N4CCOCC4)cc3)n2)cc1 ; O=C(Nc1ccc(-c2ccnc(Nc3ccc(N4CCOCC4)cc3)n2)cc1)[C@@H]1CCCN1]

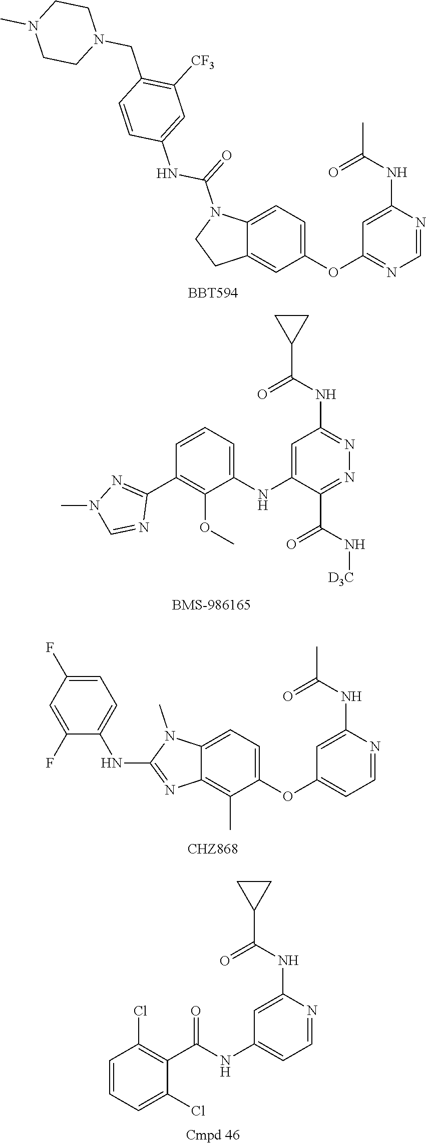

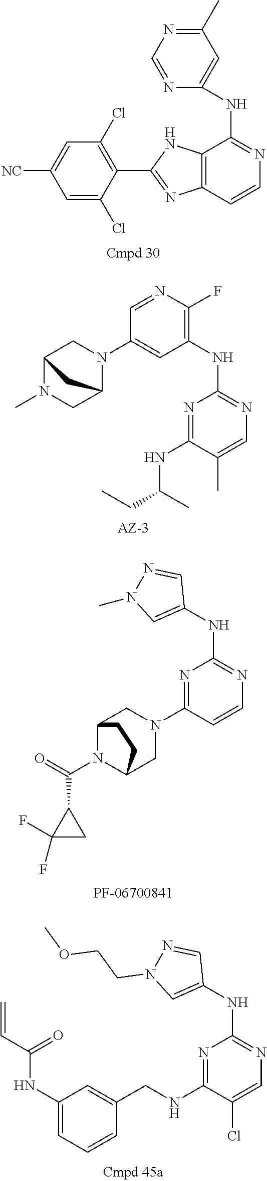

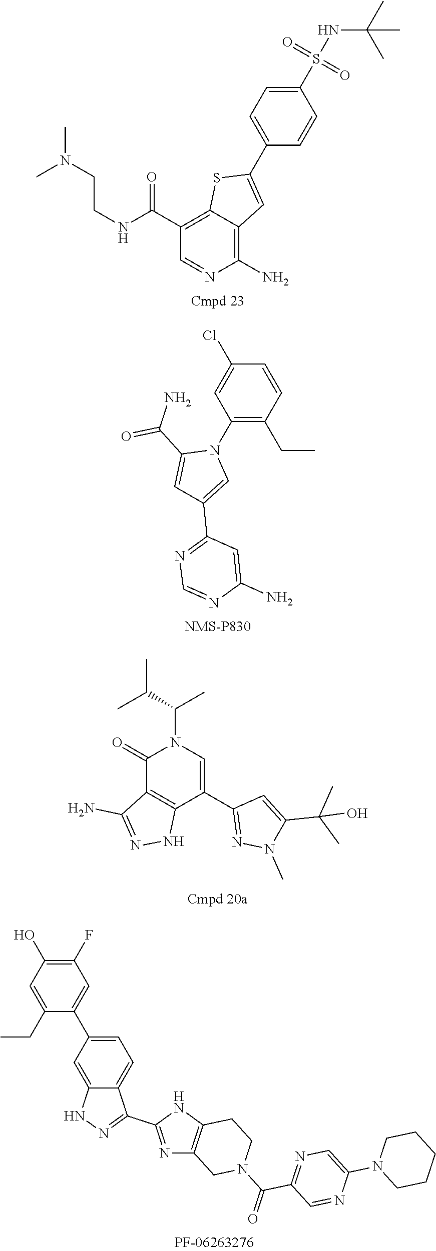

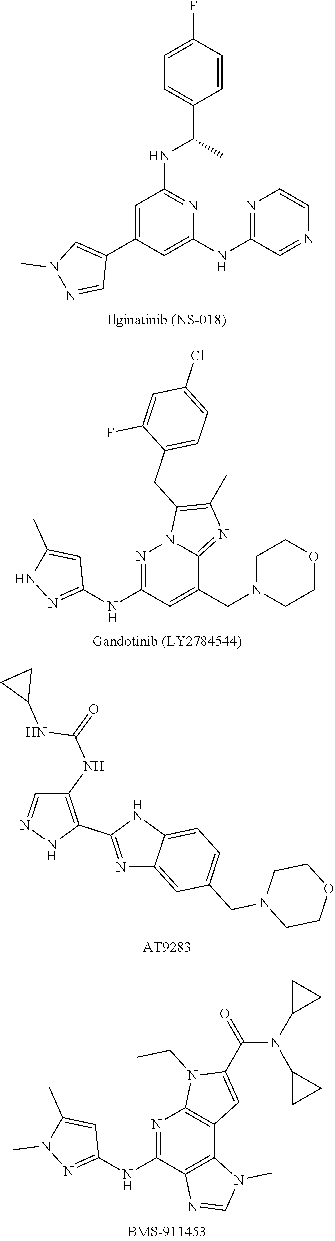 [SMILES: C[C@H](Nc1cc(-c2cnn(C)c2)cc(Nc2cnccn2)n1)c1ccc(F)cc1 ; Cc1cc(Nc2cc(CN3CCOCC3)c3nc(C)c(Cc4ccc(Cl)cc4F)n3n2)n[nH]1 ; O=C(Nc1cn[nH]c1-c1nc2cc(CN3CCOCC3)ccc2[nH]1)NC1CC1 ; CCn1c(C(=O)N(C2CC2)C2CC2)cc2c3c(ncn3C)c(Nc3cc(C)n(C)n3)nc21]

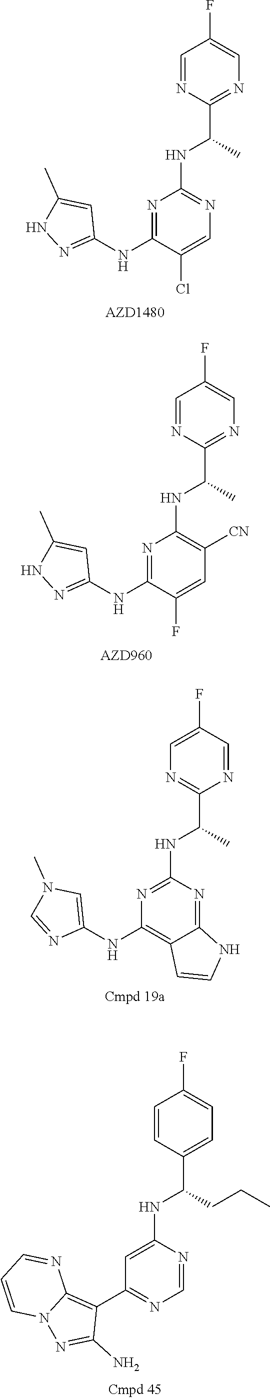 [SMILES: Cc1cc(Nc2nc(N[C@@H](C)c3ncc(F)cn3)ncc2Cl)n[nH]1 ; Cc1cc(Nc2nc(N[C@@H](C)c3ncc(F)cn3)c(C#N)cc2F)n[nH]1 ; C[C@H](Nc1nc(Nc2cn(C)cn2)c2cc[nH]c2n1)c1ncc(F)cn1 ; CCC[C@H](Nc1cc(-c2c(N)nn3cccnc23)ncn1)c1ccc(F)cc1]

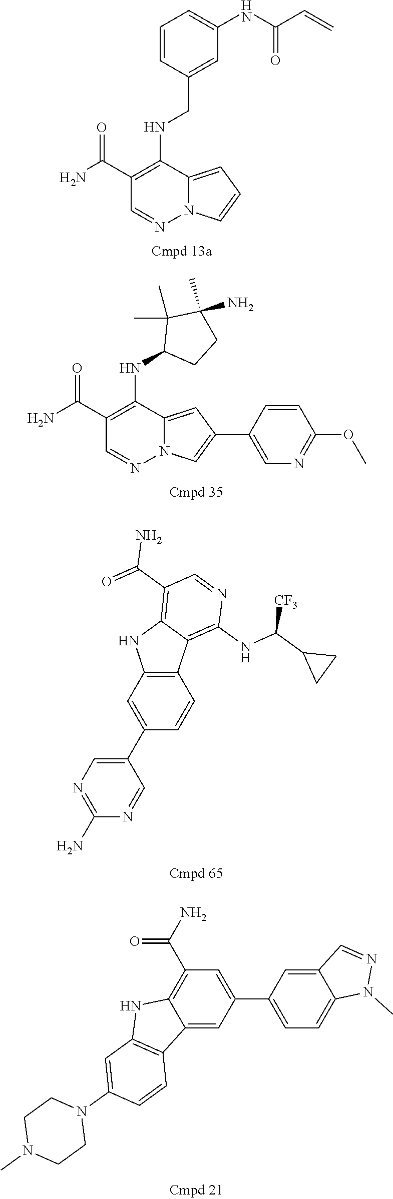

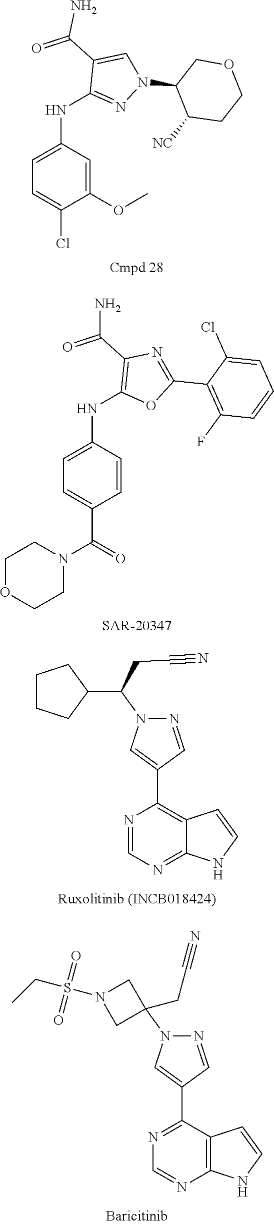

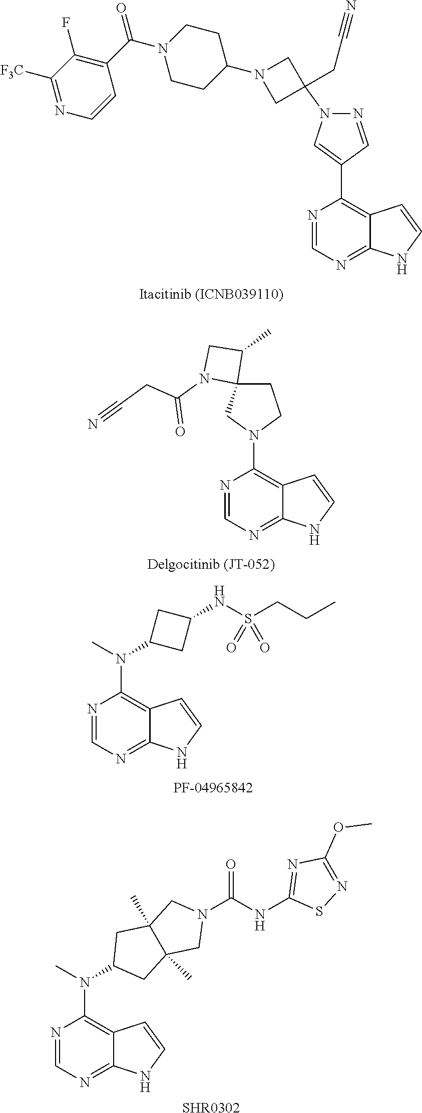

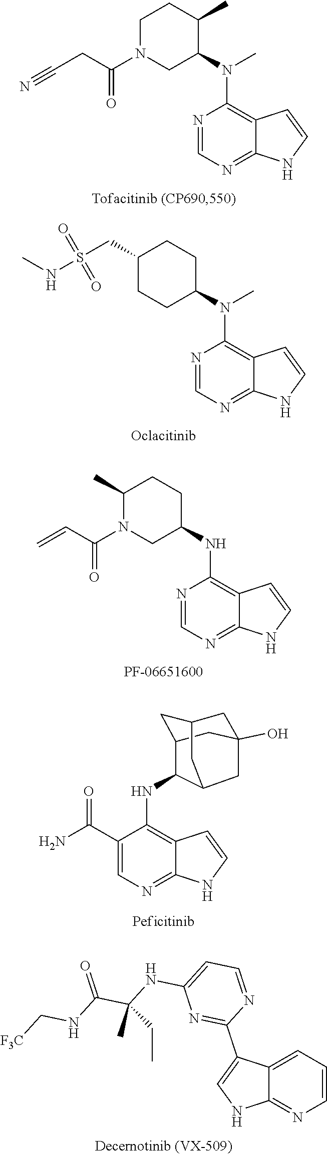

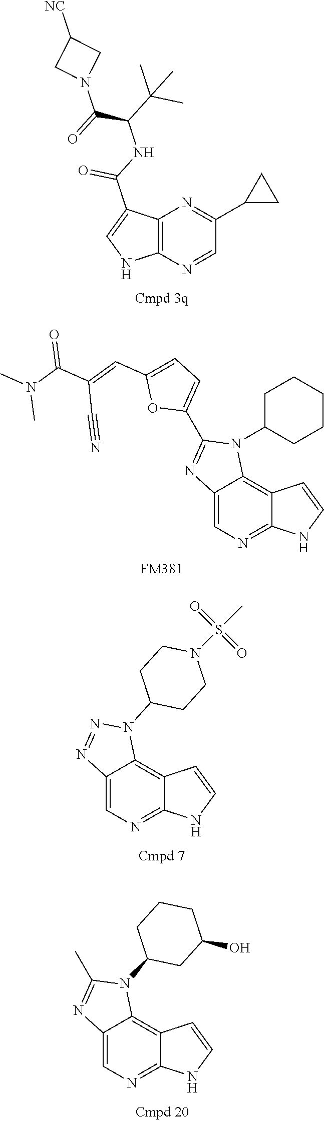

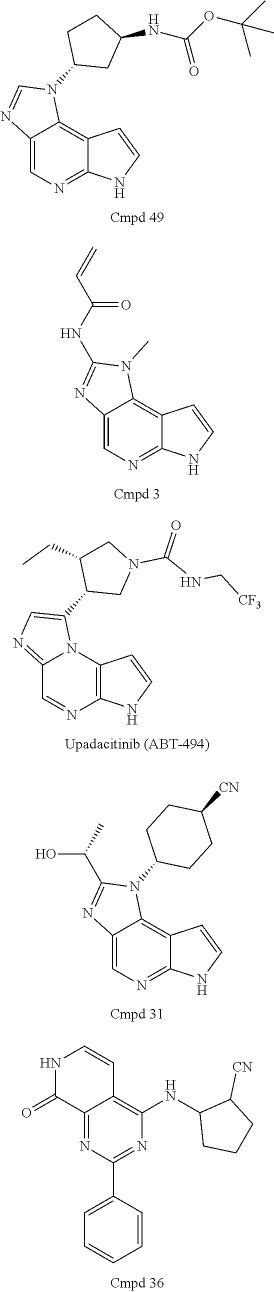 [SMILES: CC(C)(C)OC(=O)N[C@@H]1CC[C@@H](n2cnc3cnc4[nH]ccc4c32)C1 ; C=CC(=O)Nc1nc2cnc3[nH]ccc3c2n1C ; CC[C@@H]1CN(C(=O)NCC(F)(F)F)C[C@@H]1c1cnc2cnc3[nH]ccc3n12 ; C[C@@H](O)c1nc2cnc3[nH]ccc3c2n1[C@H]1CC[C@H](C#N)CC1 ; N#CC1CCCC1Nc1nc(-c2ccccc2)nc2c(=O)[nH]ccc12]

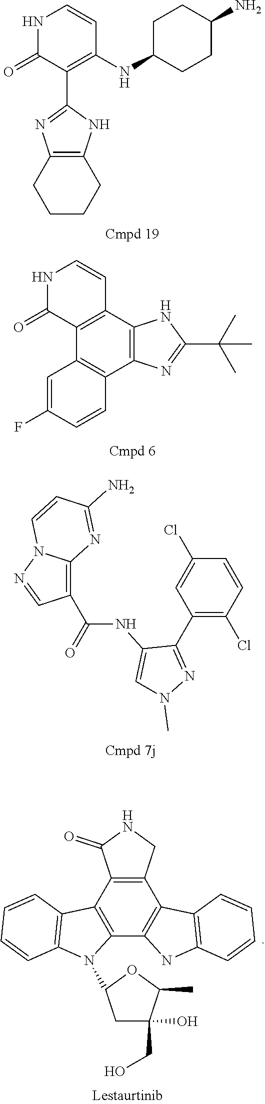

In certain embodiments, the JAK ligand include, but are not limited to Ruxolitinib, Tofacitinib (CP-690550), Baricitinib, Oclacitinib, Cerdulatinib (PRT-062070), Decernotinib (VX509), Delgocitinib (JTE-052), Fedratinib, Filgotinib (GLP0634), Gandotinib (LY2784544), Ilginatinib (NS-018), Itacitinib (INCB03911), Lestauritinib, Momelotinib (CYT387), Pacritinib (SB1578), Peficitinib, Solcitinib (GSK2586184, GLG0778), Upadacitinib (ABT-494), AT9283, AZ-3, AZ960, AZD1480, BMS-986165, BMS-911543, BSK805 (NVP-BSK805) and BVB808 (NVP-BVB808), BBT594 (NVP-BBT594), CEP-33799, CHZ868, FM381, PF-04965842, PF-06263276, PF-06651600, PF-06700841, SAR-20347, NDI-031301 (Akahane, Li, & Etchin, 2017), NDI-31232 (Masse, Miao, Greenwood, Shelley, & Kapeller, 2015), NVP-P830, SHR-0302, VR588 (Wiegman, Adcock, Rothaul, Main, & Morgan, 2015), XL019, TG101209, R333 and R348 (Deuse, et al., 2008), 3-amido pyrrolopyrazine (Cmpd 3q), pyridone containing tetracycle (Cmpd 6), triazolo-pyrrolopyridines (Cmpd 7), pyrazolopyrimidines (Cmpd 7j), 1-amino-[1,2,4]triazolo[1,5-a]pyridines (Cmpd 12), imidazolopyridines (Cmpd 19), 1-methyl-1H-imidazole derivatives (Cmpd 19a), C-2 methyl imidazopyrrolopyridines (Cmpd 20), pyrazolopyridinone (Cmpd 20a), 9H-carbazole-1-carboxamides (Cmpd 21), thianopyridines (Cmpd 23), pyrazole-4-carboxamide (Cmpd 28), imidazopyridine (Cmpd 30), hydroxyethyl imidazo-pyrrolopyridines (Cmpd 31), pyrrolopyridazines (Cmpd 35), 6-oxopyridopyrimidines (Compound 36), 2-aminopyrazolo[1,5-a]pyrimidines (Cmpd 45), cyclopropyl amides (Cmpd 46), imidazo-pyrrolopyridines (Cmpd 49), 1-amino-5H-pyrido[4,3-b]indol-4-carboxamides (Cmpd 65), Cmpd 3, Cmpd 13a, and Cmpd 45a.

In one embodiment, the JAK ligand comprises a moiety of FORMULA 1:

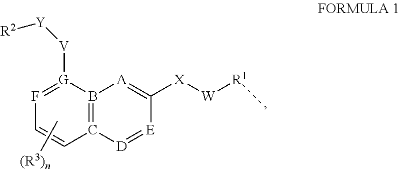

wherein

the linker moiety of the heterobifunctional compound is attached to R¹;

A and D are independently selected from CR⁴ and N, wherein

R⁴ is selected from hydrogen, halogen, optionally substituted C₁-C₈ alkyl, and optionally substituted 3-10 membered carbocyclyl;

B, C, and G are independently selected from C and N; with the proviso that at most only one of B, C, and G is N;

E and F are independently selected from null, CR⁵ and N, wherein

R⁵ is selected from hydrogen, halogen, optionally substituted C₁-C₈ alkyl, and optionally substituted 3-10 membered carbocyclyl;

X and Y are independently selected from null, or a bivalent moiety selected from null, CR⁶R⁷, CO, CO₂, CONR⁶, NR⁶, NR⁶CO, NR⁶CO₂, NR⁶C(O)NR⁷, NR⁶SO, NR⁶SO₂, NR⁶SO₂NR⁷, O, OC(O), OCO₂, OCONR⁶, S, SO, SO₂, and SO₂NR⁶, wherein

R⁶ and R⁷ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkylamino, optionally substituted 3-10 membered carbocyclyl, optionally substituted 4-10 membered heterocyclyl, optionally substituted aryl, and optionally substituted heteroaryl, or

R⁶ and R⁷ together with the atom or atoms to which they are connected form a 3-20 membered carbocyclyl ring or 4-20 membered heterocyclyl ring;

V and W are independently selected from null, carbocyclyl, heterocyclyl, aryl, and heteroaryl, which are optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring;

R¹ is connected to the “linker” moiety of the heterobifunctional compound, and is selected from null, R′—R″, R′OR″, R′SR″, R′N(R¹¹)R″, R′OC(O)R″, R′OC(O)OR″, R′OCON(R¹¹)R″, R′C(O)R″, R′C(O)OR″, R′CON(R¹¹)R″, R′S(O)R″, R′S(O)₂R″, R′SO₂N(R¹¹)R″, R′NR¹²C(O)OR″, R′NR¹²C(O)R″, R′NR¹²C(O)N(R¹¹)R″, R′NR¹²S(O)R″, R′NR¹²S(O)₂R″, and R′NR¹²S(O)₂N(R¹¹)R″, wherein

R′ and R″ are independently selected from null, optionally substituted C₁-C₈ alkyl, optionally substituted C₂-C₈ alkenyl, optionally substituted C₂-C₈ alkynyl, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused carbocyclyl, optionally substituted C₄-C₁₃ fused heterocyclyl, optionally substituted C₃-C₁₃ bridged carbocyclyl, optionally substituted C₄-C₁₃ bridged heterocyclyl, optionally substituted C₃-C₁₃ spiro carbocyclyl, optionally substituted C₄-C₁₃ spiro heterocyclyl, optionally substituted aryl, and optionally substituted heteroaryl;

R¹¹ and R¹² are independently selected from optionally substituted C₁-C₈ alkyl, optionally substituted C₂-C₈ alkenyl, optionally substituted C₂-C₈ alkynyl, optionally substituted 3-10 membered carbocyclyl, optionally substituted 4-10 membered heterocyclyl, optionally substituted aryl, and optionally substituted heteroaryl;

R′ and R″, R¹¹ and R¹², R′ and R¹¹, R′ and R¹², R″ and R¹¹, R″ and R¹², together with the atom to which they are connected, form a 3-20 membered carbocyclyl or 4-20 membered heterocyclyl ring;

R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted 3-10 membered carbocyclyl, optionally substituted 4-10 membered heterocyclyl, optionally substituted aryl, and optionally substituted heteroaryl;

R³, at each occurrence, is selected from hydrogen, halogen, optionally substituted C₁-C₈ alkyl, and optionally substituted 3-10 membered carbocyclyl; and

n is selected from 1 or 2.

In one refinement, V is Ar².

In one refinement, the JAK ligand comprises a moiety of FORMULA 1A:

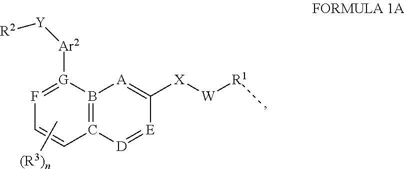

wherein

the linker moiety of the heterobifunctional compound is attached to R¹;

A, B, C, D, E, F, G, X, Y, W, R¹, R², R³, and n are the same as defined in FORMULA 1; and

Ar² is selected from null, aryl, and heteroaryl, each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring.

In another refinement, V is Ar²; and W is Ar¹. The JAK ligand comprises a moiety of FORMULA 1B:

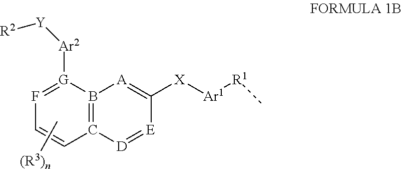

wherein

the linker moiety of the heterobifunctional compound is attached to R¹;

A, B, C, D, E, F, G, X, Y, R¹, R², R³, and n are the same as defined in FORMULA 1; and

Ar¹ and Ar² are independently selected from null, aryl, and heteroaryl, each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring.

In another refinement, A is N. The JAK ligand comprises a moiety of FORMULA 1C:

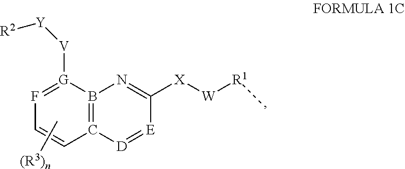

wherein

the linker moiety of the heterobifunctional compound is attached to R¹; and

B, C, D, E, F, G, V, W, X, Y, R¹, R², R³, and n are the same as defined in FORMULA 1.

In another refinement, A is N; and V is Ar². The JAK ligand comprises a moiety of FORMULA 1D:

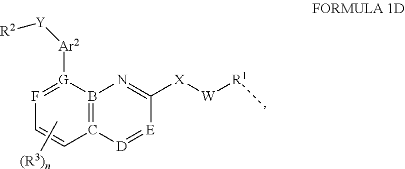

wherein

the linker moiety of the heterobifunctional compound is attached to R¹;

B, C, D, E, F, G, W, X, Y, R¹, R², R³, and n are the same as defined in FORMULA 1; and

Ar² is the same as defined in FORMULA 1A.

In another refinement, A is N; V is Ar²; and W is Ar¹.

In another refinement, the JAK ligand comprises a moiety of FORMULA 1E:

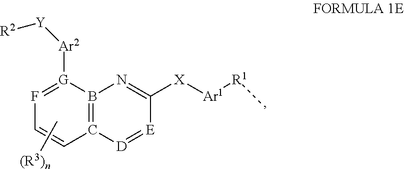

wherein

the linker moiety of the heterobifunctional compound is attached to R¹;

B, C, D, E, F, G, X, Y, R¹, R², R³, and n are the same as defined in FORMULA 1, and

Ar¹ and Ar² are the same as defined in FORMULA 1B.

In another refinement, the JAK ligand comprises a moiety of FORMULAE 1F, 1G, 1H, or 1I:

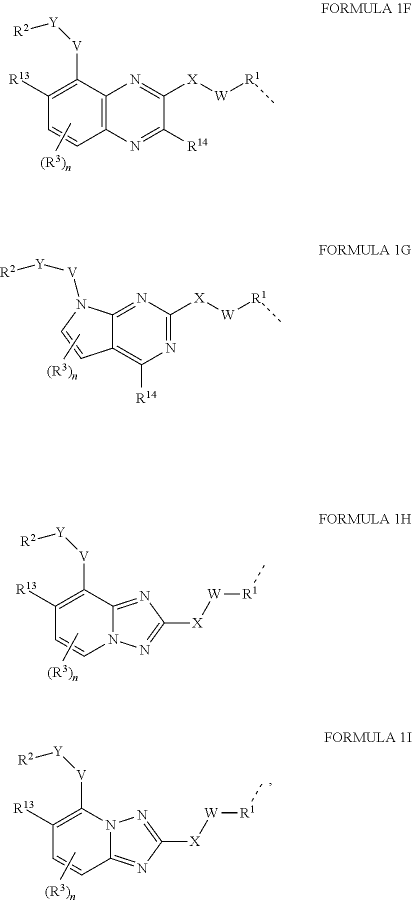

wherein

the linker moiety of the heterobifunctional compound is attached to R¹;

V, W, X, Y, R¹, R², R³, and n are the same as defined in FORMULA 1; and

R¹³ and R¹⁴ are selected from hydrogen, halogen, optionally substituted C₁-C₈ alkyl, and optionally substituted 3-10 membered carbocyclyl.

In another refinement, the JAK ligand comprises a moiety of FORMULAE 1J, 1K, 1L, or 1M:

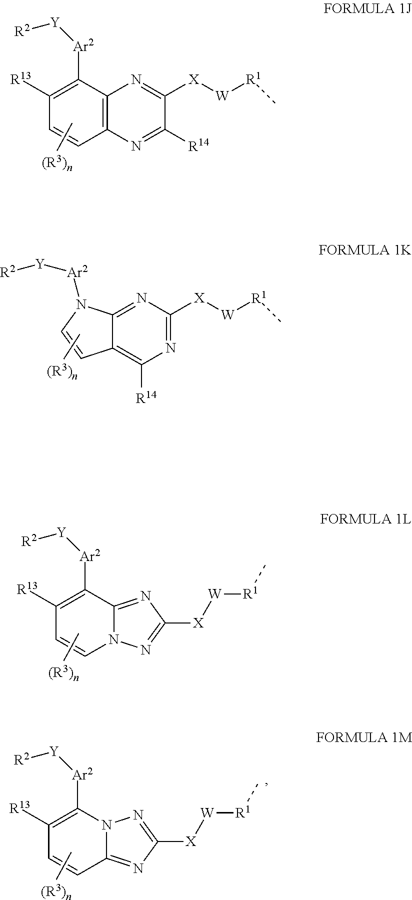

wherein

the linker moiety of the heterobifunctional compound is attached to R¹;

W, X, Y, R¹, R², R³, and n are the same as defined in FORMULA 1;

Ar² is the same as defined in FORMULA 1A; and

R¹³ and R¹⁴ are the same as defined in FORMULAE 1F, 1G, 1H or 1I.

In another refinement, the JAK ligand comprises a moiety of FORMULAE 1N, 1O, 1P, and 1Q:

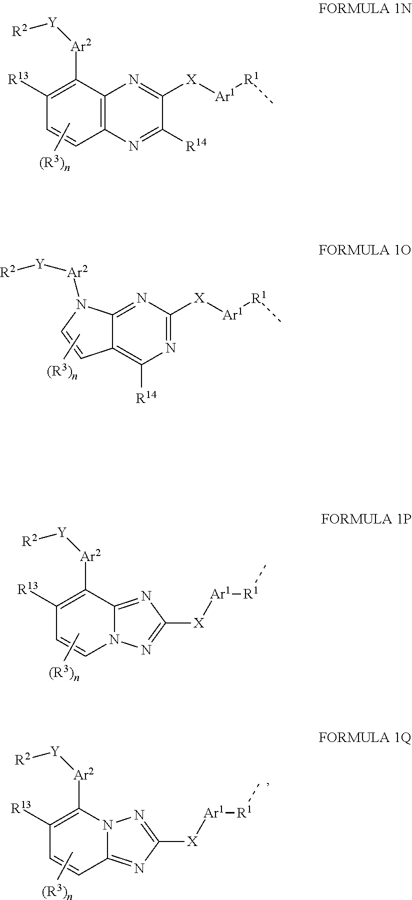

wherein

the linker moiety of the heterobifunctional compound is attached to R¹;

X, Y, R¹, R², R³, and n are the same as defined in FORMULA 1;

Ar¹ and Ar² are the same as defined in FORMULA 1B; and

R¹³ and R¹⁴ are the same as defined in FORMULAE 1F, 1G, 1H, or 1I.

In another refinement, X is selected from null, O, and NR⁶, wherein

R⁶ is selected from hydrogen, optionally substituted C₁-C₈ alkyl, and optionally substituted 3-10 membered carbocyclyl.

In another refinement, X is selected from null and NH.

In another refinement, Y is selected from null, CR^{6′}R⁷, CO, CO₂, O, SO, SO₂, and NR^{6′}, wherein

R^{6′} and R⁷ are independently selected from hydrogen, optionally substituted C₁-C₈ alkyl, optionally substituted 3-10 membered carbocyclyl, and optionally substituted 3-10 membered heterocyclyl.

In another refinement, Y is selected from null, CH₂, CO, and SO₂.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from hydrogen, halogen, oxo, CN, NO₂, OR⁸, SR⁸, NR⁸R⁹, COR⁸, CO₂R⁸, CONR⁸R⁹, SOR⁸, SO₂R⁸, SO₂NR⁸R⁹, NR¹⁰COR⁸, NR¹⁰SOR⁸, NR¹⁰SO₂R⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from hydrogen, halogen, oxo, CN, NO₂, OR⁸, NR⁸R⁹, NR¹⁰COR⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from hydrogen, CH₃, CF₃, iPr, cPr, OCH₃, OCF₃, OiPr, OcPr, F, Cl, and Br.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from H and F.

In another refinement, R¹ is selected from null, O, NH, CO, CONH, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted aryl, and optionally substituted heteroaryl.

In another refinement, R¹ is selected from null, optionally substituted 3-10 membered carbocyclyl, and optionally substituted 4-10 membered heterocyclyl.

In another refinement, R¹ is selected from null and optionally substituted 4-10 membered heterocyclyl, which contains at least one of O or N.

In another refinement, R¹ is selected from null, optionally substituted piperidinyl, optionally substituted piperazinyl, optionally substituted morpholinyl, optionally substituted pyrrolidinyl, optionally substituted tetrahydrofuranyl, optionally substituted azetidinyl, and optionally substituted oxetanyl.

In another refinement, R² is selected from hydrogen, halogen, optionally substituted C₁-C₈ alkyl, optionally substituted C₂-C₈ alkenyl, optionally substituted C₂-C₈ alkenyl, optionally substituted 3-10 membered carbocyclyl, and optionally substituted 4-10 membered heterocyclyl.

In another refinement, R² is selected from hydrogen, halogen, optionally substituted C₁-C₈ alkyl, optionally substituted 3-10 membered carbocyclyl, and optionally substituted 4-10 membered heterocyclyl.

In another refinement, R² is selected from CH₃, CF₃, iPr, cPr, F, Cl, Br, optionally substituted piperidinyl, optionally substituted optionally substituted piperazinyl, optionally substituted morpholinyl, optionally substituted pyrrolidinyl, optionally substituted tetrahydrofuranyl, optionally substituted azetidinyl, and optionally substituted oxetanyl.

In another refinement, R³, at each occurrence, R¹³ and R¹⁴ are selected from hydrogen, CH₃, CF₃, iPr, cPr, tBu, CNCH₂, F, Cl, Br, OH, NH₂, CN, CH₃, and CONH₂.

In another embodiment, the JAK ligand comprises a moiety of FORMULA 2:

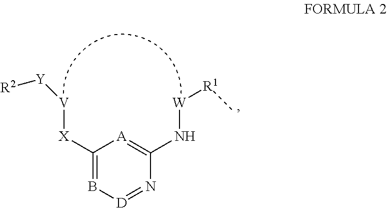

wherein

the linker moiety of the heterobifunctional compound is attached to R¹;

A, B, and D are independently selected from CR³ and N, with the proviso that not all of A, B, and D are N, wherein

R³ is selected from hydrogen, halogen, hydroxyl, amino, cyano, nitro, CONR⁴R⁵, optionally substituted C₁-C₈ alkyl, and optionally substituted 3-10 membered carbocyclyl, wherein

R⁴ and R⁵ are independently selected from hydrogen, optionally substituted C₁-C₈ alkyl, optionally substituted C₂-C₈ alkenyl, optionally substituted C₂-C₈ alkynyl, optionally substituted 3-10 membered carbocyclyl, and optionally substituted 4-10 membered heterocyclyl, or

R⁴ and R⁵ together with the atom or atoms to which they are connected form a 3-20 membered carbocyclyl ring or 4-20 membered heterocyclyl ring;

X and Y are independently selected from null, or a bivalent moiety selected from null, CR⁶R⁷, CO, CO₂, CONR⁶, NR⁶, NR⁶CO, NR⁶CO₂, NR⁶C(O)NR⁷, NR⁶SO, NR⁶SO₂, NR⁶SO₂NR⁷, O, OC(O), OCO₂, OCONR⁶, S, SO, SO₂, and SO₂NR⁶, wherein

R⁶ and R⁷ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkylamino, optionally substituted 3-10 membered carbocyclyl, optionally substituted 4-10 membered heterocyclyl, optionally substituted aryl, and optionally substituted heteroaryl, or

R⁶ and R⁷ together with the atom or atoms to which they are connected form a 3-20 membered carbocyclyl ring or 4-20 membered heterocyclyl ring;

V and W are independently selected from null, carbocyclyl, heterocyclyl, aryl, and heteroaryl, which are optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring;

When neither of V and W is null, V and W together with the substituents to which they are connected optionally form a 10-30 membered macrocyclic ring; When W is null and V is not null, V and R¹ together with the substituents to which they are connected optionally form a 10-30 membered macrocyclic ring; When V is null and W is not null, W and R² together with the substituents to which they are connected optionally form a 10-30 membered macrocyclic ring; or When W and V are null, R¹ and R² together with the substituents to which they are connected optionally form a 10-30 membered macrocyclic ring;

R¹ is connected to the “linker” moiety of the heterobifunctional compound, and is selected from null, R′—R″, R′OR″, R′SR″, R′NR¹¹R″, R′OC(O)R″, R′OC(O)OR″, R′OCON(R¹¹)R″, R′C(O)R″, R′C(O)OR″, R′CON(R¹¹)R″, R′S(O)R″, R′S(O)₂R″, R′SO₂N(R¹¹)R″, R′NR¹²C(O)OR″, R′NR¹²C(O)R″, R′NR¹²C(O)N(R¹¹)R″, R′NR¹²S(O)R″, R′NR¹²S(O)₂R″, and R′NR¹²S(O)₂N(R¹¹)R″, wherein

R′ and R″ are independently selected from null, optionally substituted C₁-C₈ alkyl, optionally substituted C₂-C₈ alkenyl, optionally substituted C₂-C₈ alkynyl, optionally substituted C₁-C₈ alkylene, optionally substituted C₂-C₈ alkenylene, optionally substituted C₂-C₈alkynylene, optionally substituted 3-10 membered carbocyclyl, optionally substituted 4-10 membered heterocyclyl, optionally substituted C₃-C₁₃ fused carbocyclyl, optionally substituted C₄-C₁₃ fused heterocyclyl, optionally substituted C₃-C₁₃ bridged carbocyclyl, optionally substituted C₄-C₁₃ bridged heterocyclyl, optionally substituted C₃-C₁₃ spiro carbocyclyl, optionally substituted C₄-C₁₃ spiro heterocyclyl, optionally substituted aryl, and optionally substituted heteroaryl;

R¹¹ and R¹² are independently selected from optionally substituted C₁-C₈ alkyl, optionally substituted C₂-C₈ alkenyl, optionally substituted C₂-C₈ alkynyl, optionally substituted 3-10 membered carbocyclyl, optionally substituted 4-10 membered heterocyclyl, optionally substituted aryl, and optionally substituted heteroaryl; or

R′ and R″, R¹¹ and R¹², R′ and R¹¹, R′ and R¹², R″ and R¹¹, R″ and R¹², together with the atom to which they are connected, form a 3-20 membered carbocyclyl or 4-20 membered heterocyclyl ring; and

R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted 3-10 membered carbocyclyl, optionally substituted 4-10 membered heterocyclyl, optionally substituted aryl, and optionally substituted heteroaryl;

In another refinement, V is Ar².

In another refinement, the JAK ligand comprises a moiety of FORMULA 2A:

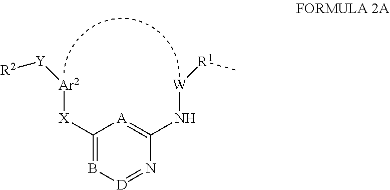

wherein

the linker moiety of the heterobifunctional compound is attached to R¹;

A, B, D, X, Y, W, R¹, and R² are the same as defined in FORMULA 2;

Ar² is selected from null, aryl, and heteroaryl, each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring; and

When neither of W and Ar² is null, W and Ar² together with the substituents to which they are connected optionally form a 10-30 membered macrocyclic ring; When W is null and Ar² is not null, Ar² and R¹ together with the substituents to which they are connected optionally form a 10-30 membered macrocyclic ring; When Ar² is null and W is not null, W and R² together with the substituents to which they are connected optionally form a 10-30 membered macrocyclic ring; or When W and Ar² are null, R¹ and R² together with the substituents to which they are connected optionally form a 10-30 membered macrocyclic ring.

In another refinement, V is Ar²; and W is Ar¹. The JAK ligand comprises a moiety of FORMULA 2B:

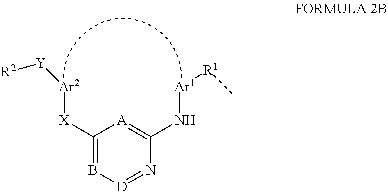

wherein

the linker moiety of the heterobifunctional compound is attached to R¹;

A, B, D, X, Y, R¹, and R² are the same as defined in FORMULA 2; and

Ar¹ and Ar² are independently selected from null, aryl, and heteroaryl, each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein

R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or

R⁸ and R⁹, R⁸ and R¹⁰ together with the atom to which they are connected form a 4-20 membered heterocyclyl ring; and

When neither of Ar¹ and Ar² is null, Ar¹ and Ar² together with the substituents to which they are connected optionally form a 10-30 membered macrocyclic ring; When Ar¹ is null and Ar² is not null, Ar² and R¹ together with the substituents to which they are connected optionally form a 10-30 membered macrocyclic ring; When Ar² is null and Ar¹ is not null, Ar¹ and R² together with the substituents to which they are connected optionally form a 10-30 membered macrocyclic ring; or When Ar¹ and Ar² are null, R¹ and R² together with the substituents to which they are connected optionally form a 10-30 membered macrocyclic ring.

In another refinement, the JAK ligand comprises a moiety of FORMULAE 2C, 2D, 2E or 2F:

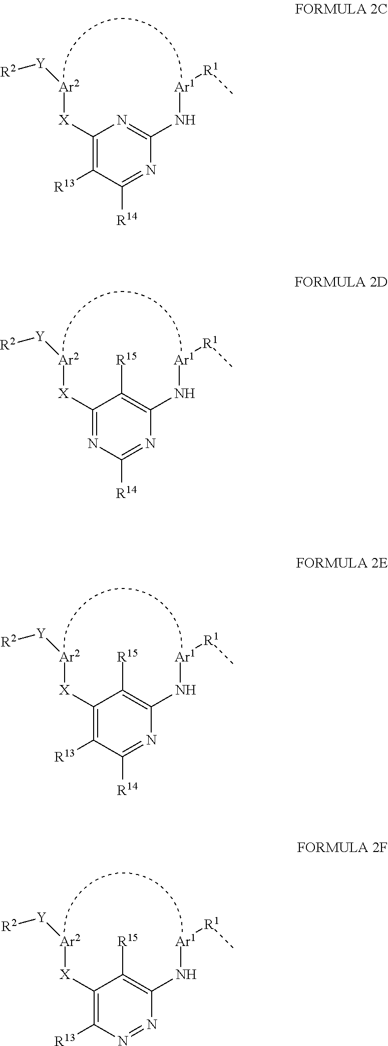

wherein

the linker moiety of the heterobifunctional compound is attached to R¹;

X, Y, Ar¹, Ar², R¹, and R² are the same as defined in FORMULA 2; and

R¹³, R¹⁴ and R¹⁵ are selected from hydrogen, halogen, hydroxyl, amino, cyano, nitro, CONR⁴R⁵, optionally substituted C₁-C₈ alkyl, and optionally substituted 3-10 membered carbocyclyl, wherein

R⁴ and R⁵ are independently selected from hydrogen, optionally substituted C₁-C₈ alkyl, optionally substituted C₂-C₈ alkenyl, optionally substituted C₂-C₈ alkynyl, optionally substituted 3-10 membered carbocyclyl, and optionally substituted 4-10 membered heterocyclyl, or

R⁴ and R⁵ together with the atom or atoms to which they are connected form a 3-20 membered carbocyclyl ring or 4-20 membered heterocyclyl ring.

In another refinement, the JAK ligand comprises a moiety of FORMULAE 2G, 2H, 2I, 2J, 2K, 2L, 2M, 2N, 2O, 2P, 2Q, 2R or 2S:

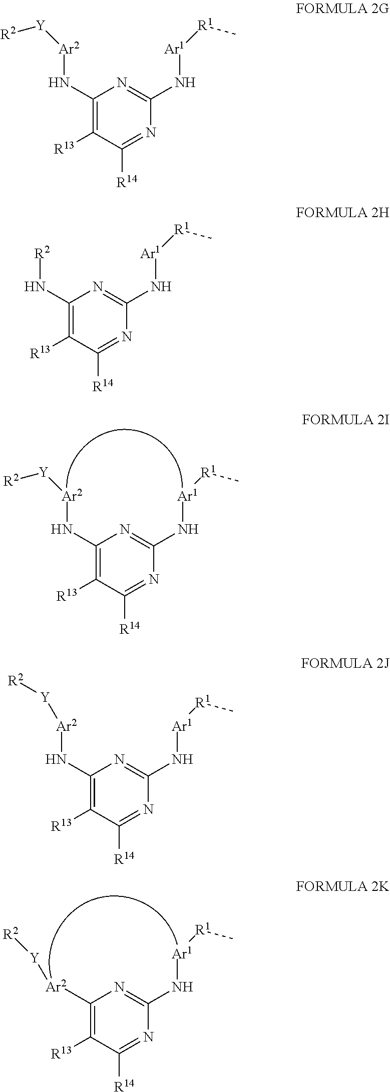

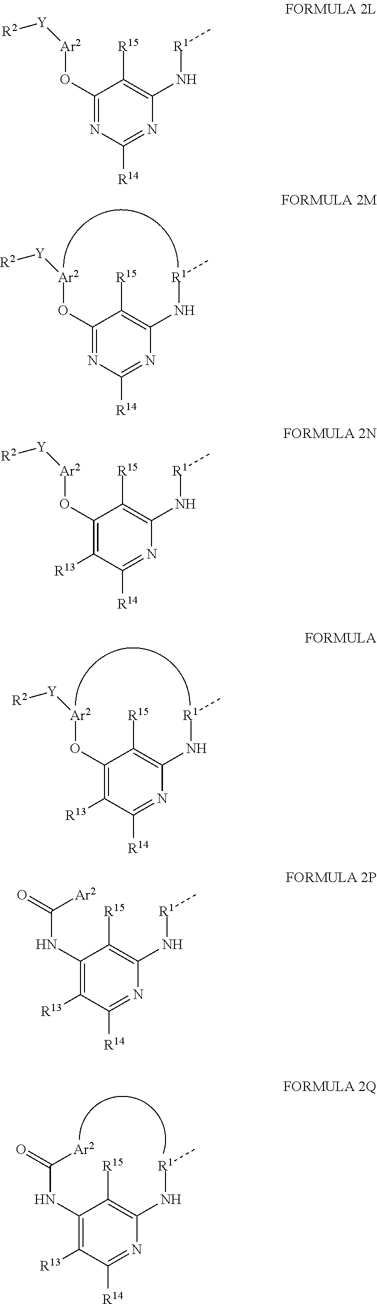

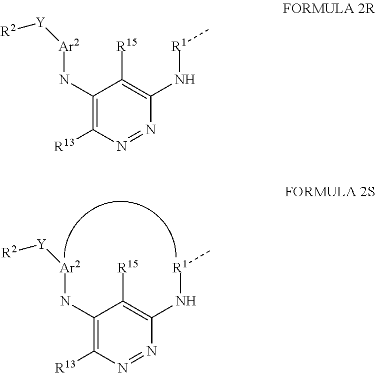

wherein

the linker moiety of the heterobifunctional compound is attached to R¹;

Y, R¹ and R² are the same as defined in FORMULA 2;

Ar¹ and Ar² are the same as FORMULA 2B; and

R¹³, R¹⁴ and R¹⁵ are the same as FORMULAE 2C, 2D, 2E or 2F.

In another refinement, Y is selected from null, CR⁶R⁷, CO, CO₂, CONR⁶, NR⁶CO, NR⁶C(O)NR⁷, O, SO, SO₂, SO₂NR⁶ and NR⁶, wherein

R⁶ and R⁷ are independently selected from hydrogen, optionally substituted C₁-C₈ alkyl, optionally substituted 3-10 membered carbocyclyl, and optionally substituted 3-10 membered heterocyclyl.

In another refinement, Y is selected from null, CH₂, CO, CONH, NR⁶C(O), NR⁶C(O)NR⁷, SO₂ and SO₂NH.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from hydrogen, halogen, oxo, CN, NO₂, OR⁸, SR⁸, NR⁸R⁹, COR⁸, CO₂R⁸, CONR⁸R⁹, SOR⁸, SO₂R⁸, SO₂NR⁸R⁹, NR¹⁰COR⁸, NR¹⁰SOR⁸, NR¹⁰SO₂R⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from hydrogen, halogen, oxo, CN, NO₂, OR⁸, NR⁸R⁹, NR¹⁰COR⁸, optionally substituted C₁-C₈ alkyl, optionally substituted 3-10 membered carbocyclyl, and optionally substituted 4-10 membered heterocyclyl, wherein

R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or

R⁸ and R⁹, R⁸ and R¹⁰ together with the atom to which they are connected form a 4-10 membered heterocyclyl ring.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from H, CH₃, CF₃, iPr, cPr, OCH₃, OCF₃, OiPr, OcPr, F, Cl, and Br.

In another refinement, Ar¹ and Ar² are independently selected from null, monocyclic aryl, monocyclic heteroaryl, bicyclic aryl, bicyclic heteroaryl, tricyclic aryl, and tricyclic heteroaryl, each of which is substituted with R² and optionally substituted with one or more substituents independently selected from H and F.

In another refinement, R¹ is selected from null, O, NH, CO, CONH, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted aryl, and optionally substituted heteroaryl.

In another refinement, R¹ is selected from null, O, NH, optionally substituted 3-10 membered carbocyclyl, and optionally substituted 4-10 membered heterocyclyl.

In another refinement, R¹ is selected from null, O, NH, and optionally substituted 4-10 membered heterocyclyl, which contains at least one of O or N.

In another refinement, R¹ is selected from null, O, NH, optionally substituted piperidinyl, optionally substituted piperazinyl, optionally substituted morpholinyl, optionally substituted pyrrolidinyl, optionally substituted tetrahydrofuranyl, optionally substituted azetidinyl, and optionally substituted oxetanyl.

In another refinement, R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted C₁-C₈ alkoxy, optionally substituted 3-10 membered carbocyclyl, and optionally substituted 4-10 membered heterocyclyl, optionally substituted aryl, and optionally substituted heteroaryl.

In another refinement, R² is selected from hydrogen, halogen, optionally substituted C₁-C₈alkyl, optionally substituted 3-10 membered carbocyclyl, optionally substituted 4-10 membered heterocyclyl, optionally substituted aryl, and optionally substituted heteroaryl.

In another refinement, R² is selected from hydrogen, CH₃, CF₃, iPr, cPr, tBu, CNCH₂, F, Cl, Br, optionally substituted piperidinyl, optionally substituted optionally substituted piperazinyl, optionally substituted morpholinyl, optionally substituted pyrrolidinyl, optionally substituted tetrahydrofuranyl, optionally substituted azetidinyl, and optionally substituted oxetanyl, optionally substituted phenyl, optionally substituted triazolyl, optionally substituted pyridinyl, optionally substituted pyrimidinyl, optionally substituted pyrazinyl, optionally substituted triazinyl, optionally substituted fruranyl, optionally substituted oxazolyl, optionally substituted pyrrolyl, optionally substituted imidazolyl, optionally substituted triazolyl, optionally substituted oxadiazolyl, optionally substituted thiophenyl, optionally substituted thiazolyl, and optionally substituted thiadiazolyl.

In another refinement, R¹³, R¹⁴ and R¹⁵ are independently selected from hydrogen, CH₃, CF₃, iPr, cPr, tBu, CNCH₂, F, Cl, Br, OH, NH₂, CN, CH₃, and CONH₂.

In another embodiment, the JAK ligand is derived from any of the following:

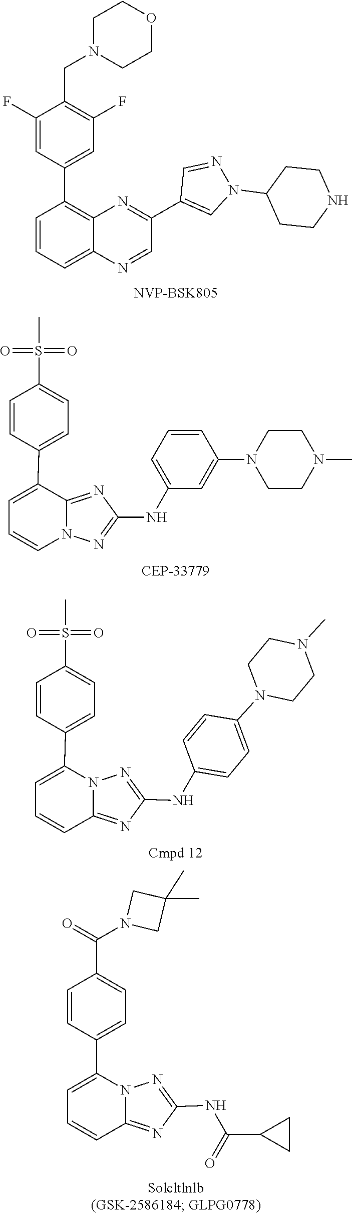

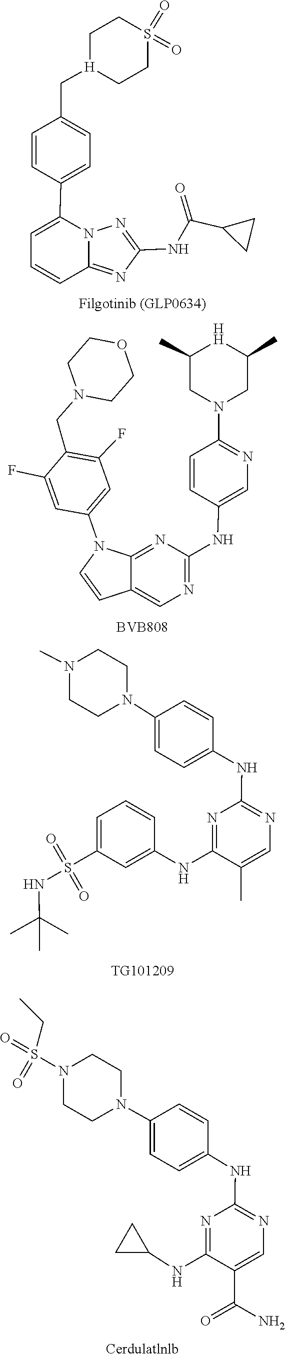

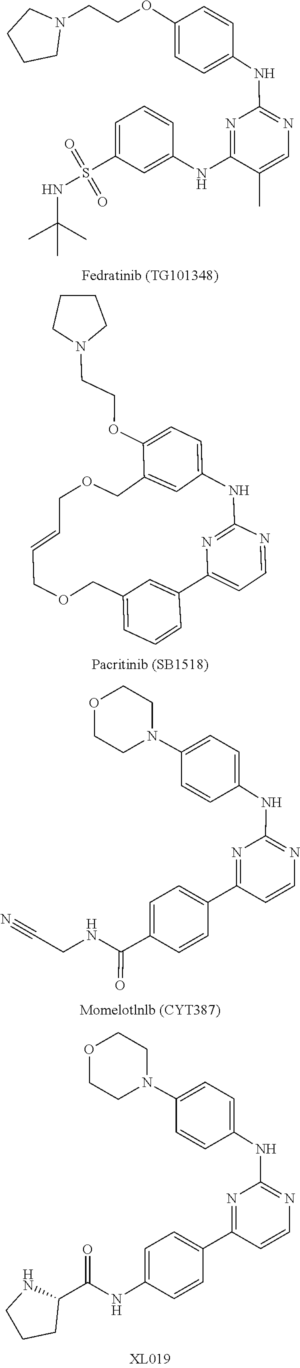

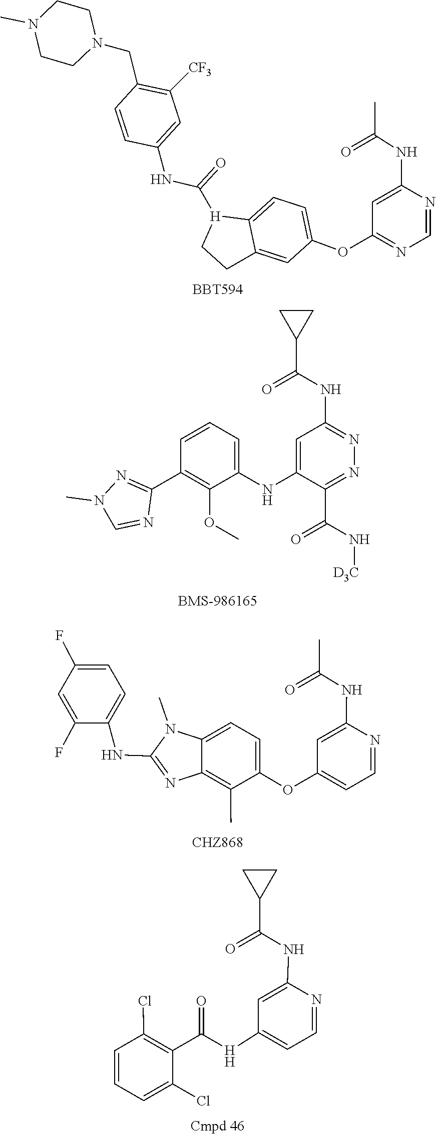

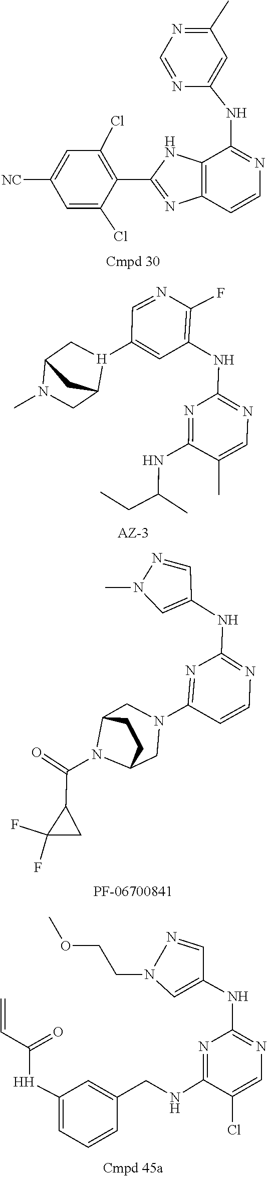

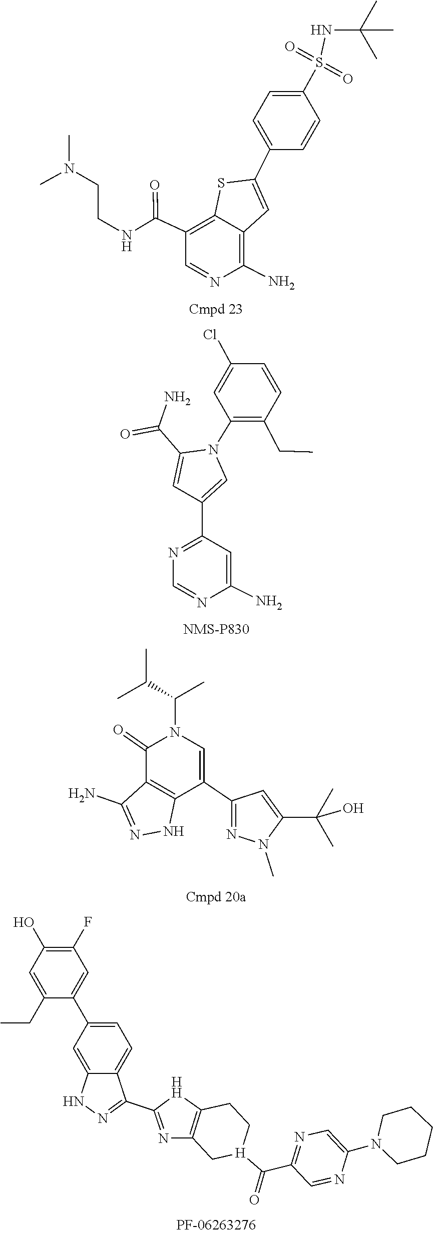

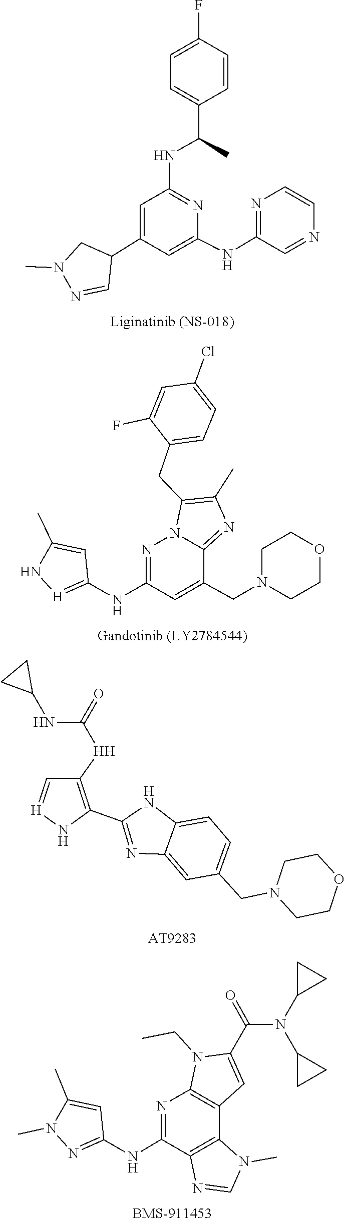

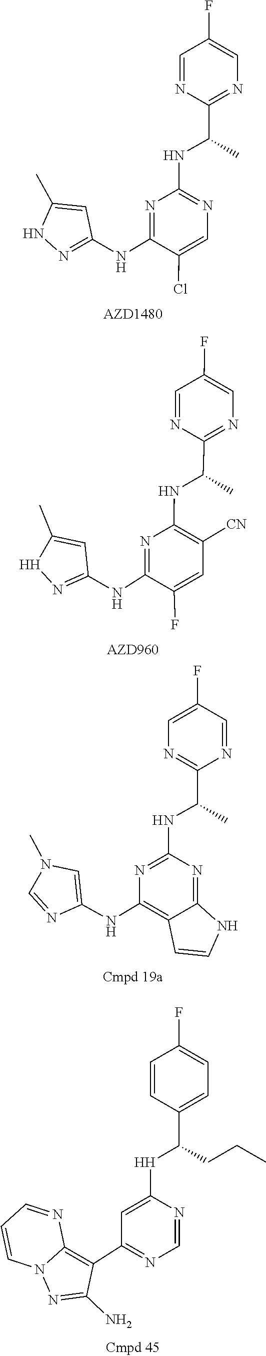

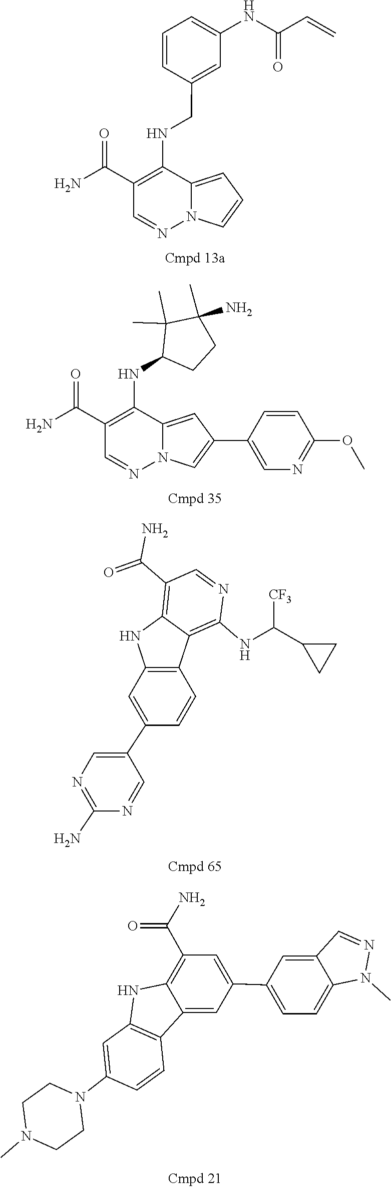

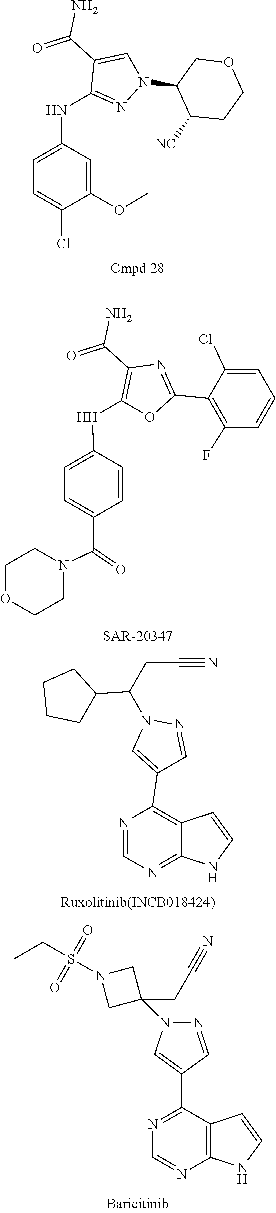

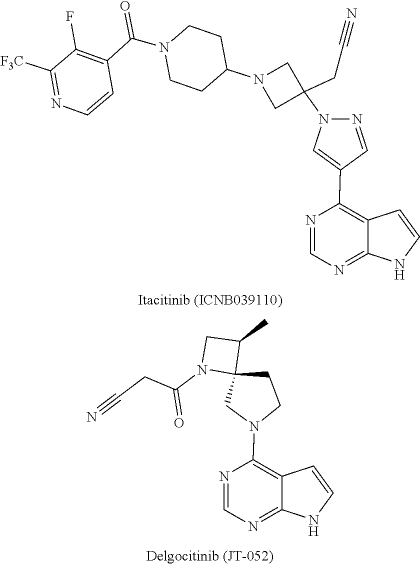 [SMILES: N#CCC1(n2cc(-c3ncnc4[nH]ccc34)cn2)CN(C2CCN(C(=O)c3ccnc(C(F)(F)F)c3F)CC2)C1 ; C[C@@H]1CN(C(=O)CC#N)[C@]12CCN(c1ncnc3[nH]ccc13)C2]

In another embodiment, the JAK ligand is derived from any of the following: NVP-BSK805, Cmpd 12, and TG101209 (preferably, NVP-BSK805 and Cmpd 12).

In another embodiment, the JAK ligand is derived from the following JAK inhibitors: NDI-031301, NDI-31232, VR588, R333 and R348.

In another embodiment, the JAK ligand is selected from the group consisting of:

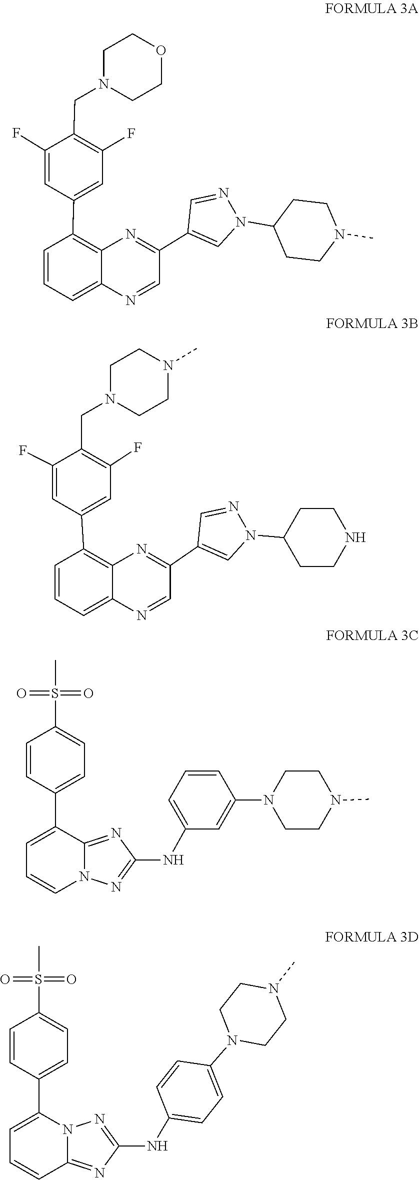

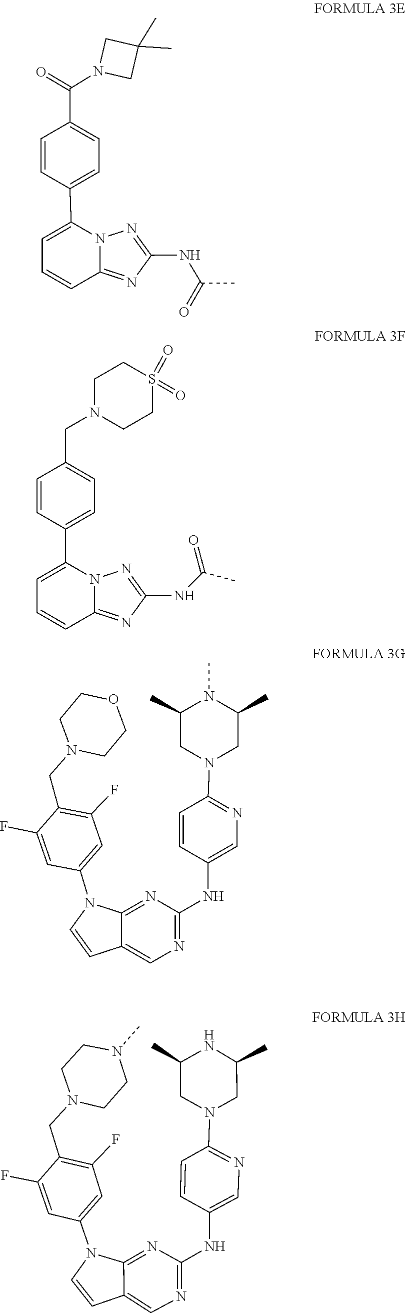

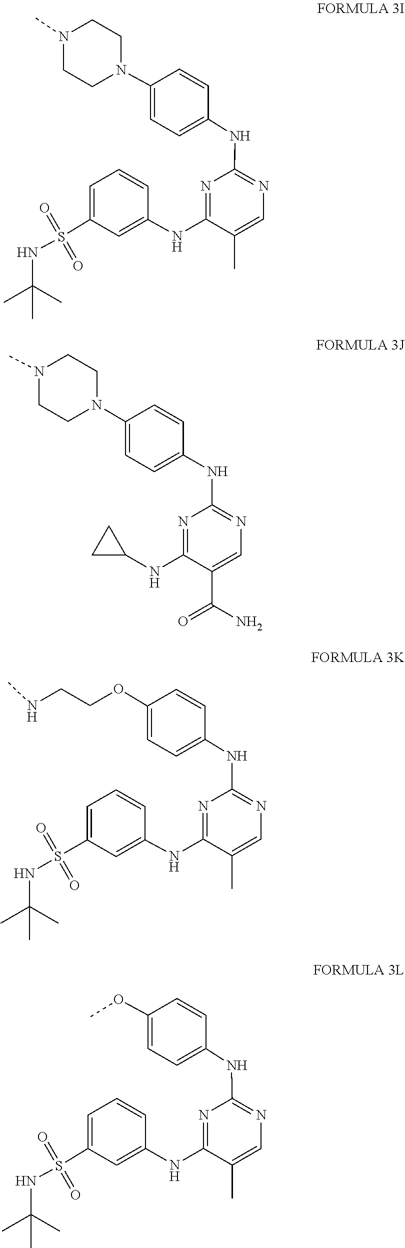

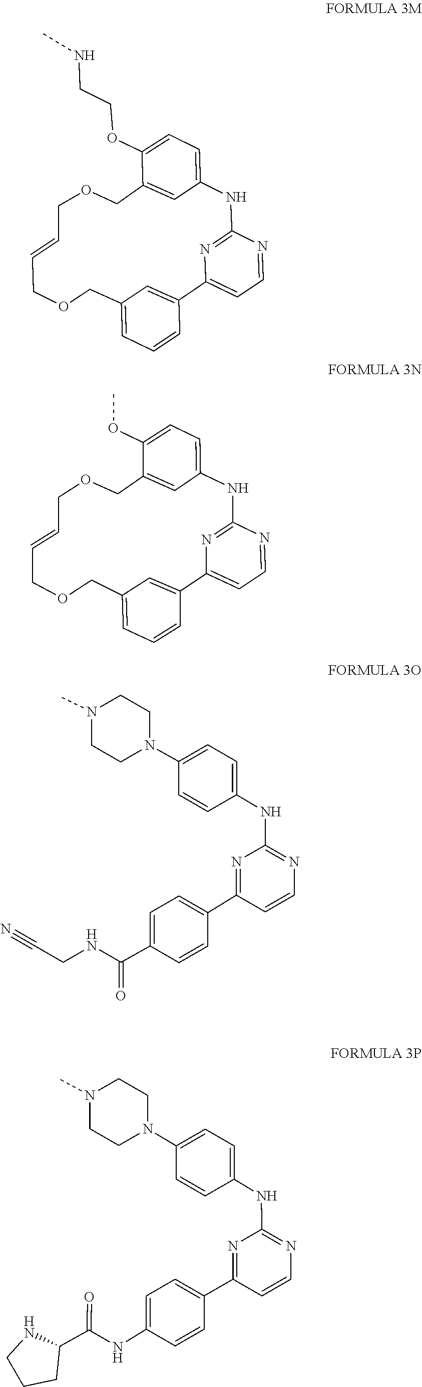

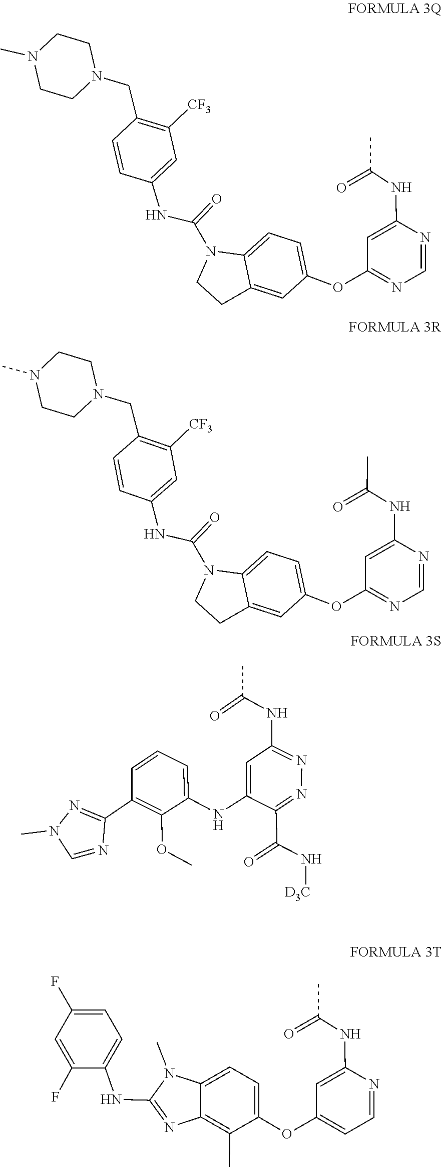

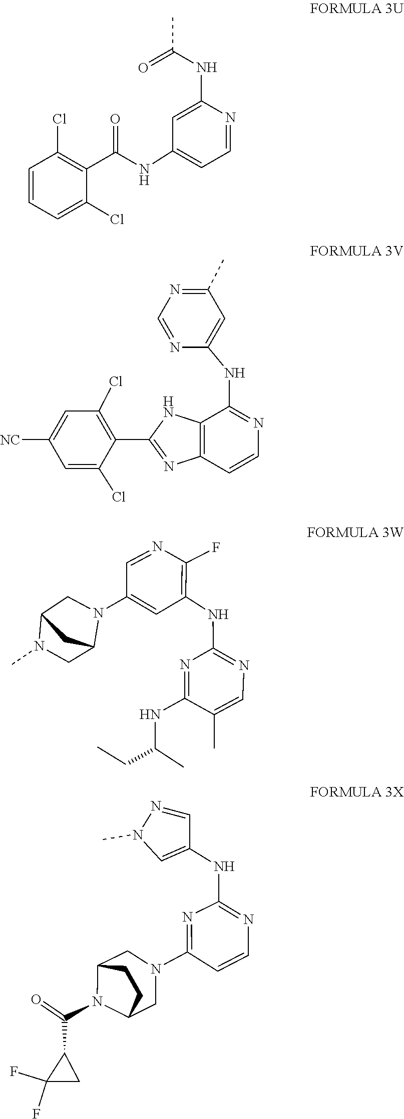

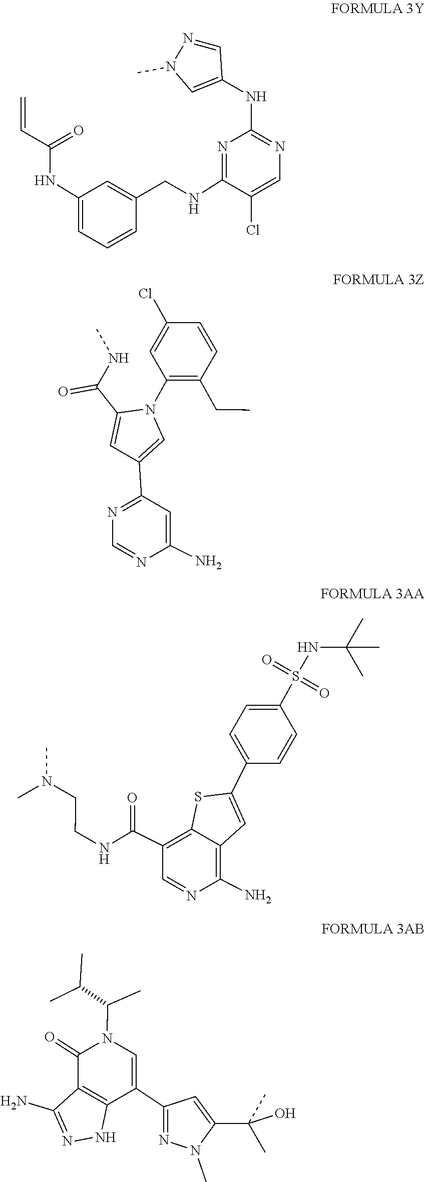

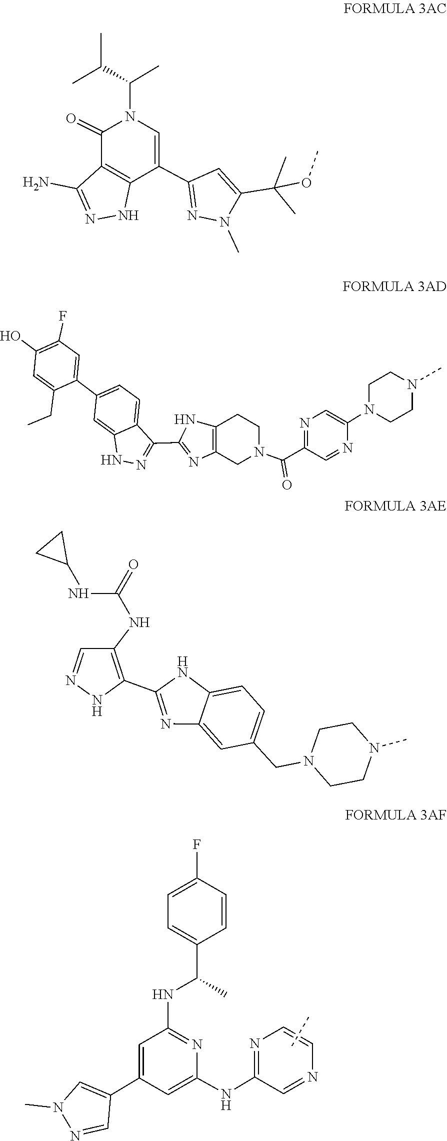

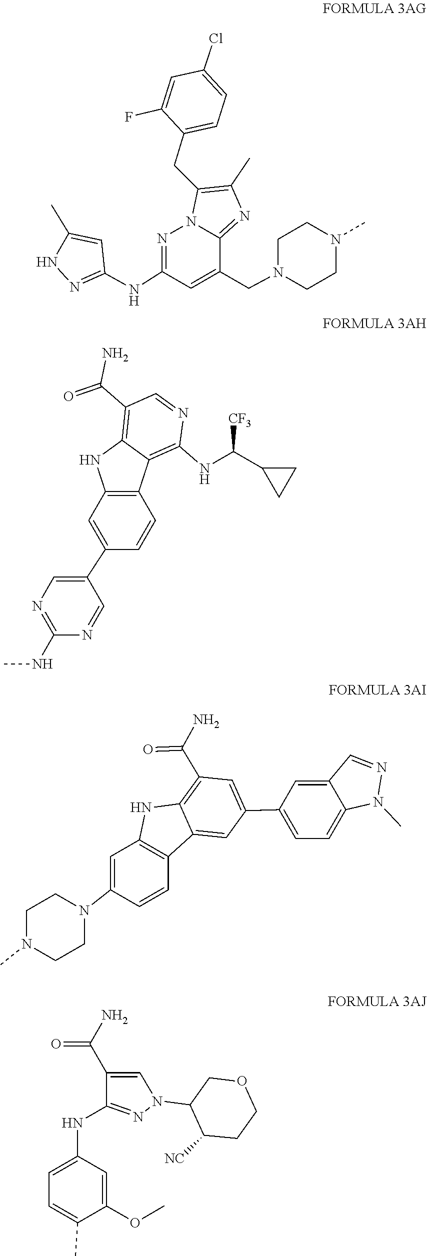

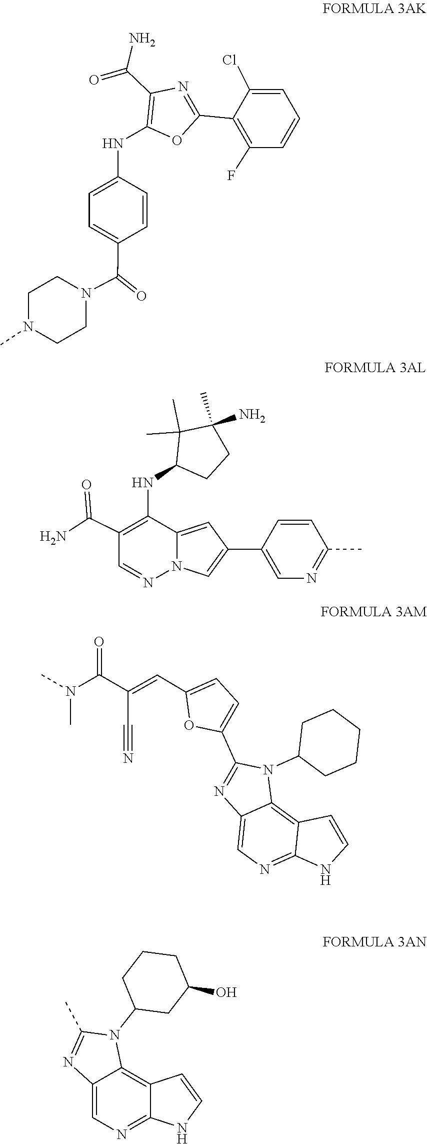

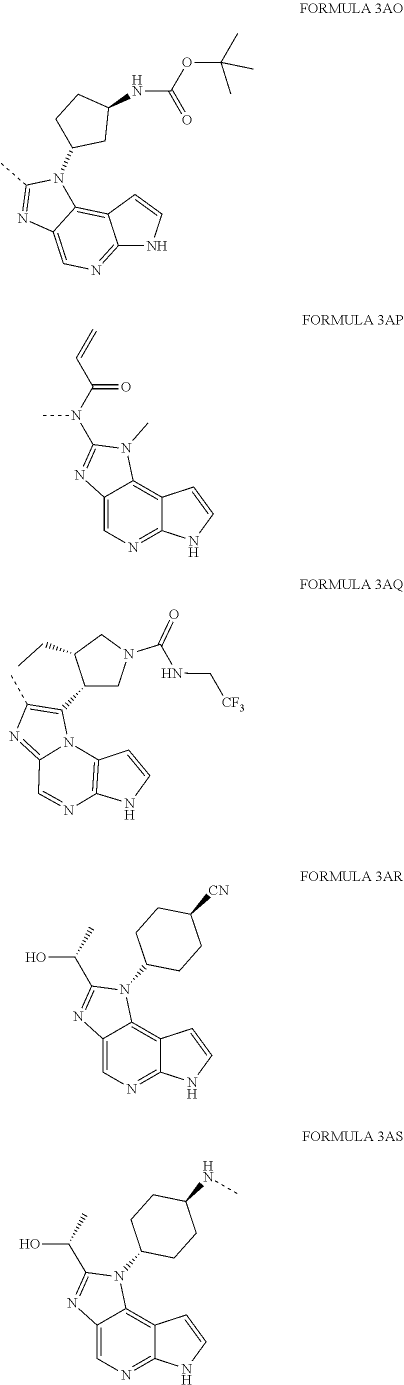

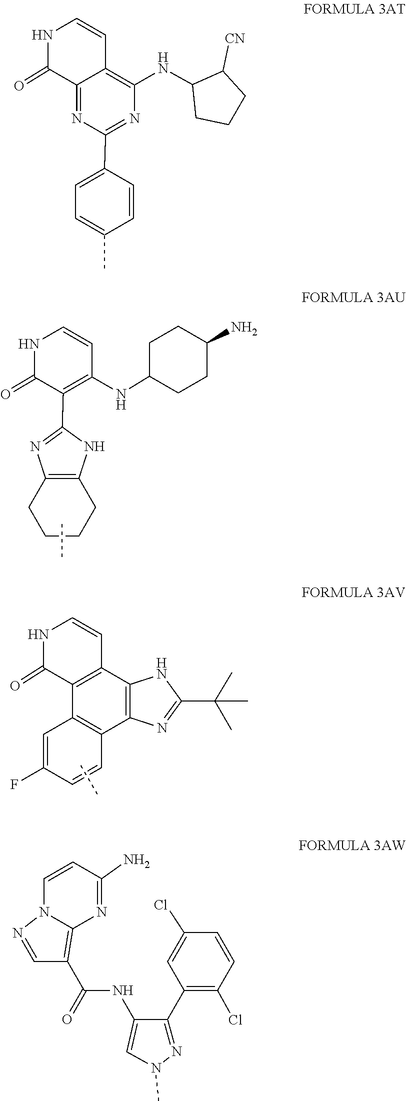

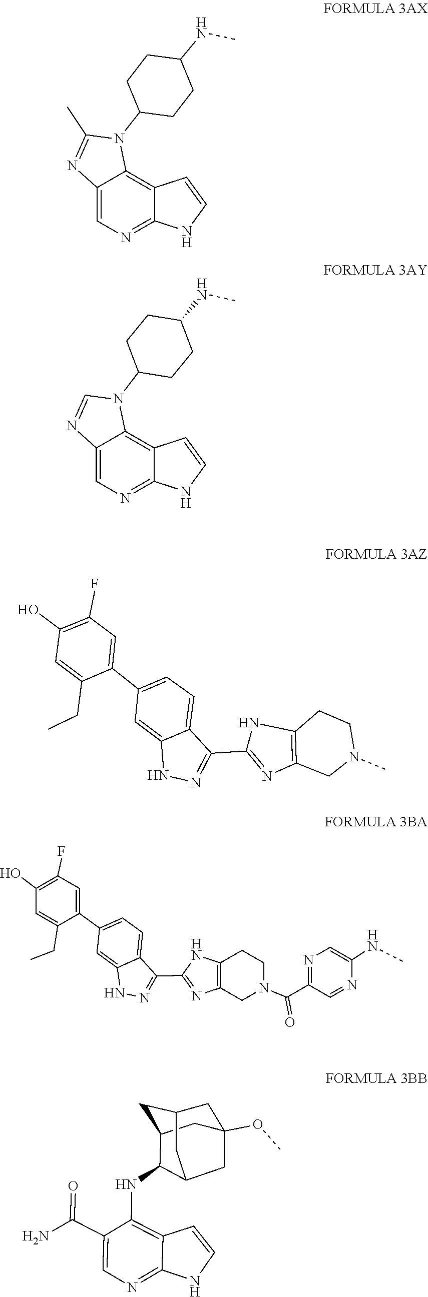

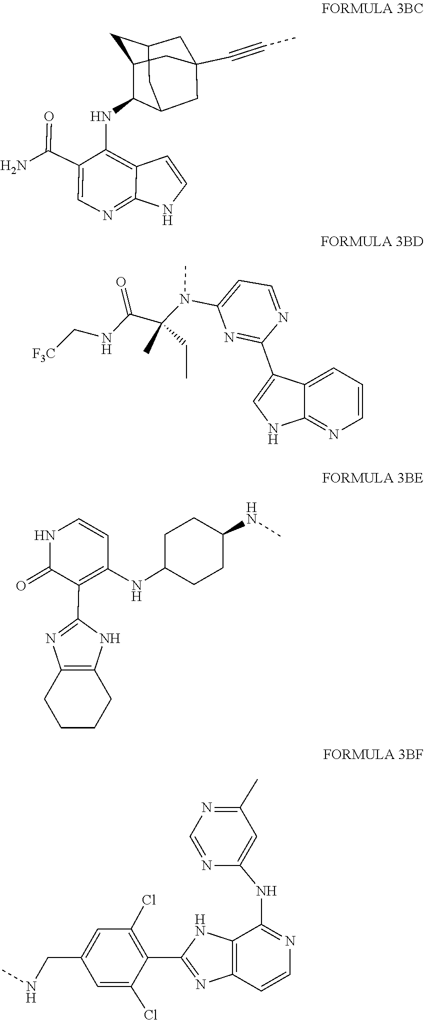

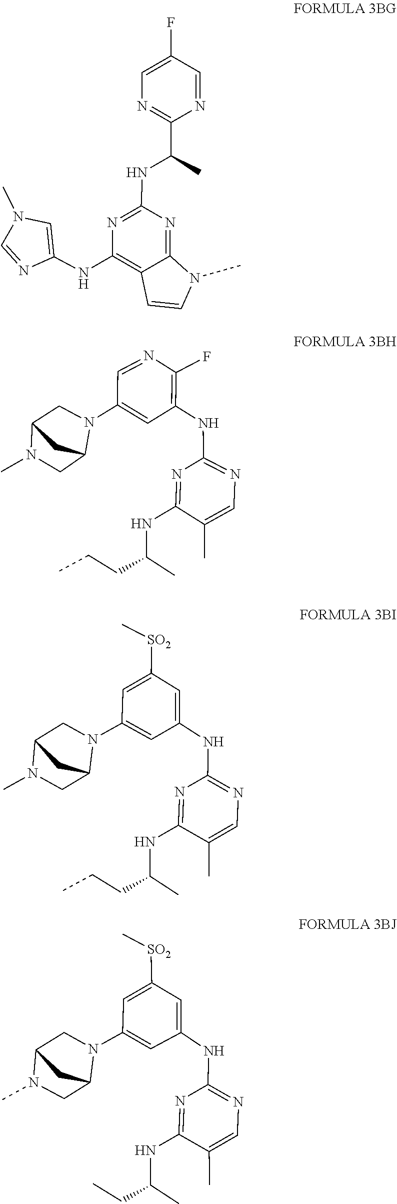

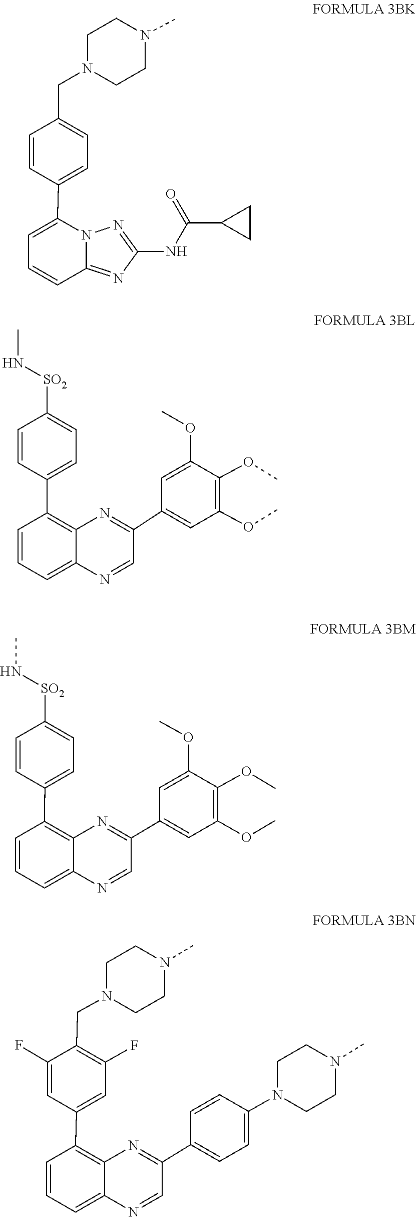

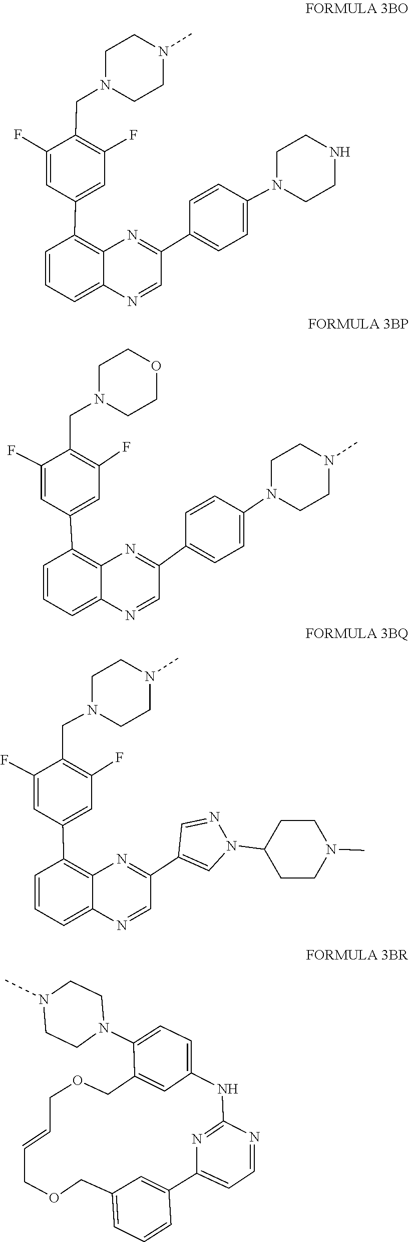

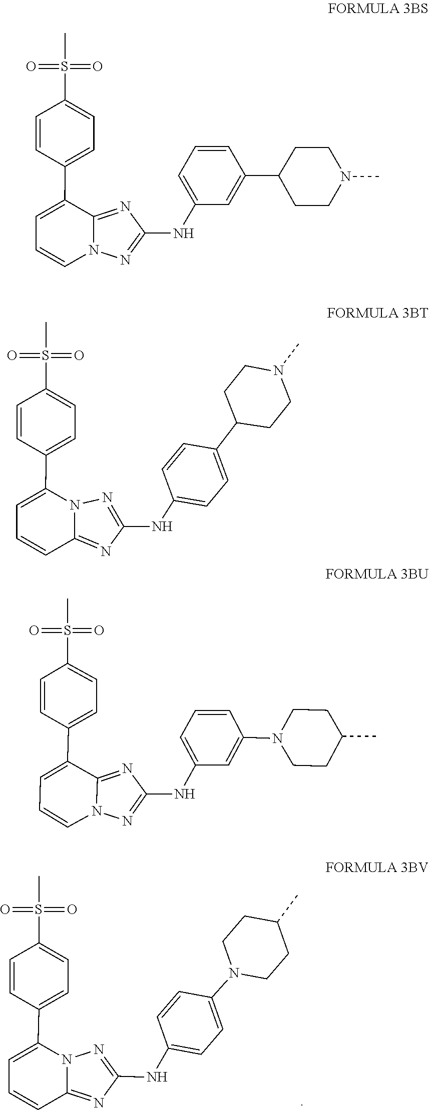

Degradation Tag

As used herein, the term “degradation tag” refers to a compound, which associates with or binds to an ubiquitin ligase for recruitment of the corresponding ubiquitination machinery to JAK or is a hydrophobic group or a tag that leads to misfolding of the JAK protein and subsequent degradation at the proteasome or loss of function.

In some embodiments, the degradation tag is a moiety selected from the group consisting of FORMULAE 5A, 5B, 5C, and 5D:

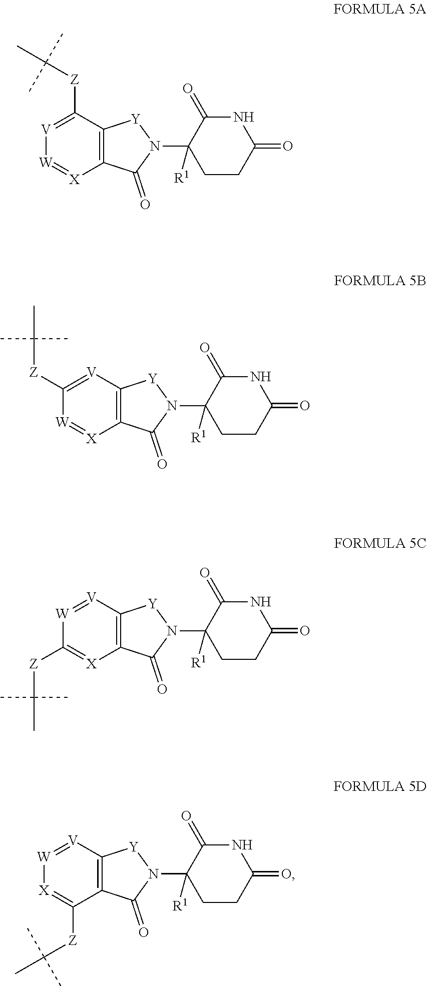

wherein

V, W, and X are independently selected from CR² and N;

Y is selected from —CO—, —CR³R⁴—, —N═CR³—, and —N═N—; preferably, Y is selected from —CO—, —CH₂—, and —N═N—;

Z is selected from null, CO, CR⁵R⁶, NR⁵, O, C≡C, optionally substituted C₁-C₁₀ alkylene, optionally substituted C₂-C₁₀ alkenyl, and optionally substituted C₂-C₁₀ alkynyl, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, NH, O, and C≡C.

R¹, R², R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl, or

R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl, or

R⁵ and R⁶ together with the atom to which they are connected form a 3-6 membered carbocyclyl, or 4-6 membered heterocyclyl.

In another embodiment, the degradation tag is a moiety selected from the group consisting of FORMULAE 5A, 5B, 5C, and 5D:

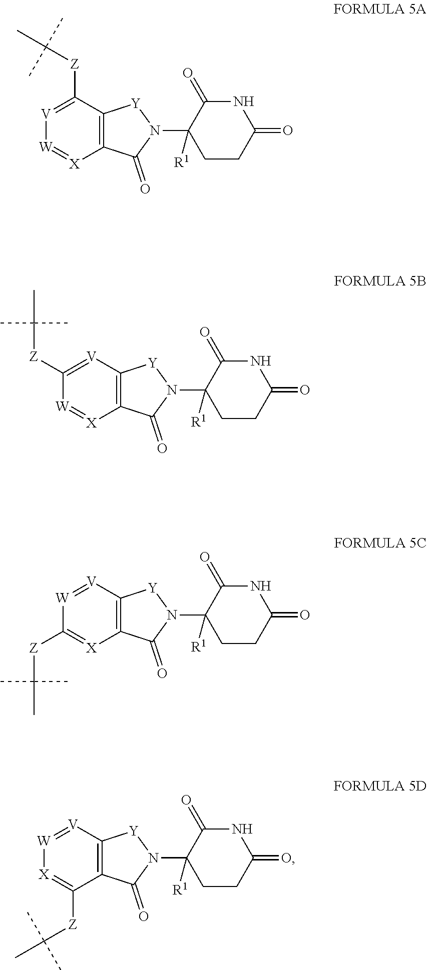

wherein

V, W, and X are independently selected from CR² and N;

Y is selected from CO, CR³R⁴, and N═N;

Z is selected from null, CO, CR⁵R⁶, NR⁵, O, optionally substituted C₁-C₁₀ alkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹, R², R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl, or

R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl, or

R⁵ and R⁶ together with the atom to which they are connected form a 3-6 membered carbocyclyl, or 4-6 membered heterocyclyl.

In another embodiment, R¹, R², R³, R⁴, R⁵ and R⁶ are hydrogen.

In another embodiment, the degradation tag is a moiety of FORMULAE 5B or 5C and wherein Y is —CR³R⁴— or CO.

In another embodiment, when the degradation tag is a moiety of FORMULAE 5B or 5C and wherein Y is CO, Z is selected from NR⁵ (preferably, Y is NH), O, or CR⁵R⁶ (preferably, CH₂).

In another embodiment, when the degradation tag is a moiety of FORMULAE 5C and wherein Y is —CR³R⁴— (preferably, Y is —CH₂—), Z is CR⁵R⁶ (preferably, CH₂).

In another embodiment, when the degradation tag is a moiety of FORMULAE 5B and wherein Y is —CR³R⁴— (preferably, Y is —CH₂—), Z is O.

In another embodiment, the degradation tag is a moiety selected from the group consisting of FORMULAE 5E, 5F, 5G, 5H, and 5I:

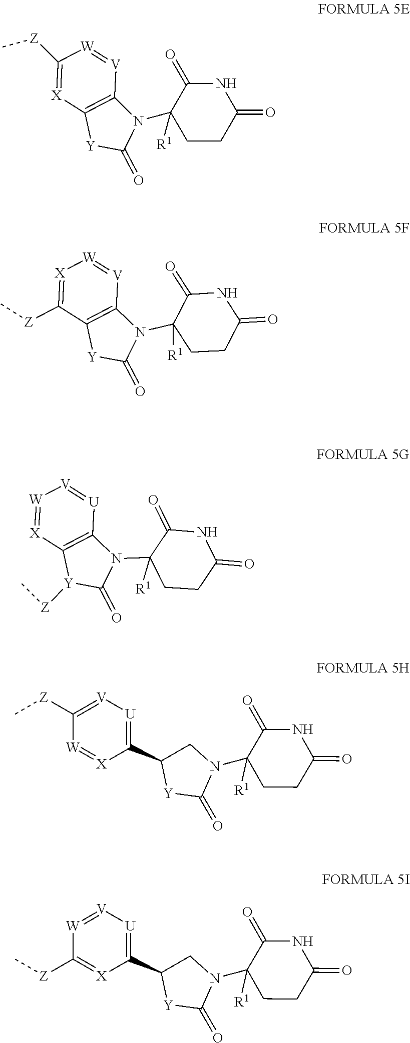

wherein

U, V, W, and X are independently selected from CR² and N;

Y is selected from —N—, —CR³═, CR³R⁴, NR³ and O; preferably, Y is selected from —N—, —CH₂—, —NH—, —N(CH₃)— and —O—;

Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;

R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;

R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl; and

R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl.

In another embodiment, when the degradation tag is a moiety of FORMULAE 5G, Y is selected from —N— and —CR³═; preferably, Y is N.

In another embodiment, when the degradation tag is a moiety of FORMULAE 5E, 5F, 5H, or 5I, Y is selected from —CR³R⁴—, —NR³—, and —O—.; preferably, Y is selected from CH₂, NH, N(CH₃) and O.

In one embodiment, the degradation tag is a moiety selected from the group consisting of FORMULAE 5J, 5K, 5L, 5M, 5N, 5O, 5P, and 5Q:

wherein

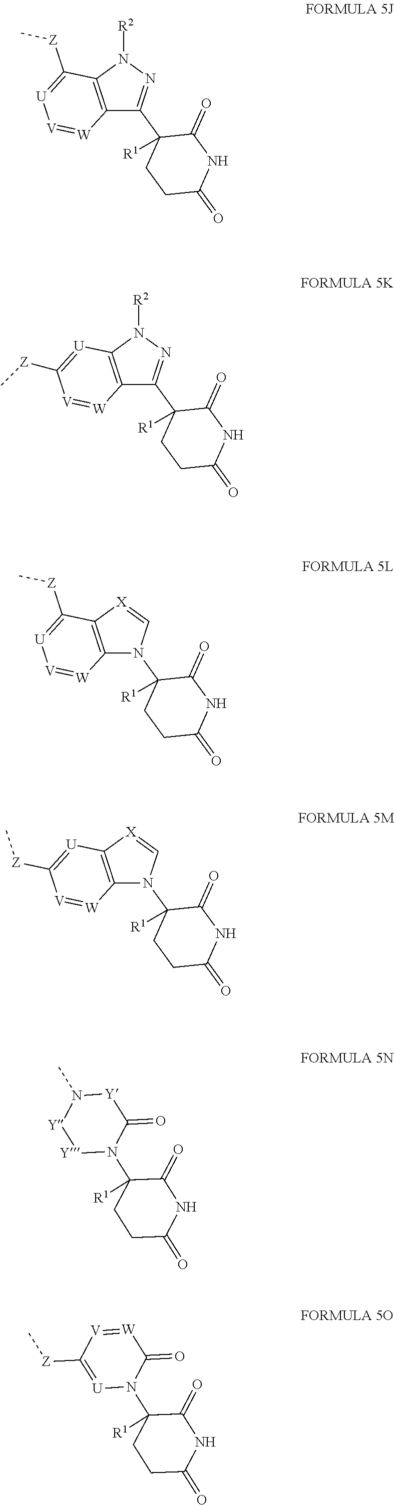

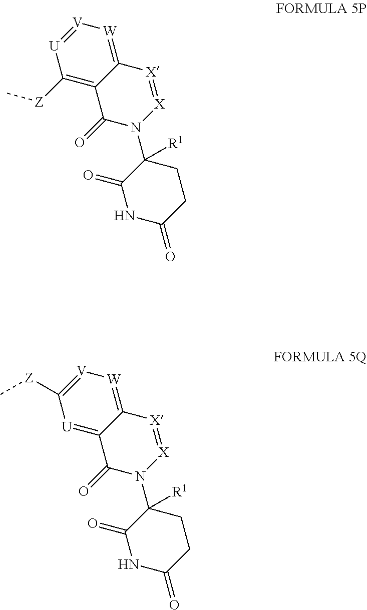

X′ are independently selected from CR² and N;

Y′, Y″, and Y′″ are independently selected from CR³R⁴;

U, V, W, Y, X, Z, R¹, R², R³ and R⁴ are defined as in FORMULAE 5E, 5F, 5G, 5H, or 5I;

R′ is selected from hydrogen, optionally substituted C1-C6 alkyl, optionally substituted 3 to 6 membered carbocyclyl, and optionally substituted 4 to 6 membered heterocyclyl.

In one embodiment, the degradation tag is a moiety of FORMULA 6A:

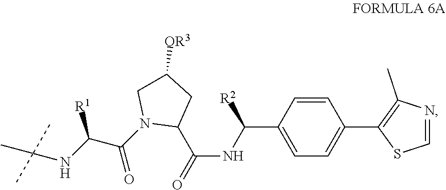

wherein

R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl; and

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂.

In another embodiment, the degradation tag is a moiety of FORMULAE 6B, 6C, and 6D:

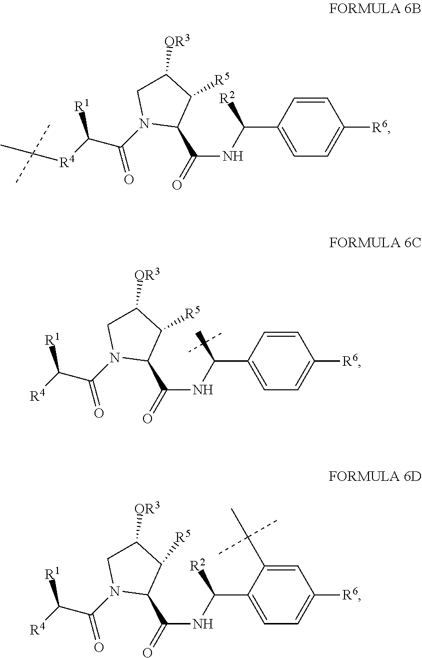

wherein

R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl;

R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂, and

R⁴ is selected from NR⁷R⁸

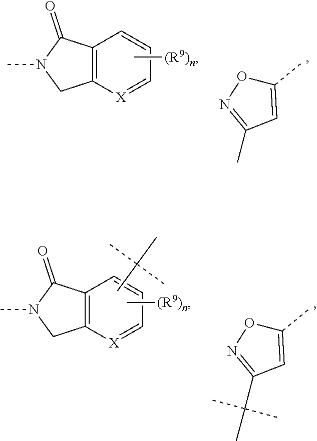

optionally substituted C₁-C₈alkoxy, optionally substituted 3-10 membered carbocyclyl, optionally substituted 4-10 membered heterocyclyl, optionally substituted aryl, and optionally substituted heteraryl, in which

R⁷ is selected from hydrogen, optionally substituted C₁-C₈alkyl, optionally substituted C₁-C₈cycloalkyl, optionally substituted C₁-C₈alkyl-CO, optionally substituted C₁-C₈cycloalkyl-CO, optionally substituted C₁-C₈cycloalkyl-C₁-C₈alkyl-CO, optionally substituted 4-10 membered heterocyclyl-CO, optionally substituted 4-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;

R⁸ is selected from hydrogen, optionally substituted C₁-C₈alkyl, and optionally substituted C₁-C₈cycloalkyl;

R⁹, at each occurrence, is independently selected from hydrogen, halogen, cyano, optionally substituted C₁-C₈alkyl, optionally substituted C₁-C₈cycloalkyl, optionally substituted C₁-C₈heterocycloalkyl, optionally substituted C₁-C₈alkoxy, optionally substituted C₁-C₈cycloalkoxy, halo substituted C₁-C₈alkyl, halo substituted C₁-C₈cycloalkyl, halo substituted C₁-C₈alkoxl, halo substituted C₁-C₈cycloalkoxy, and halo substituted C₁-C₈heterocycloalkyl;

X is selected from CH and N; and

n is 0, 1, 2, 3, or 4;

R⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl, preferably, halogen, cyano, optionally substituted imidazole, optionally substituted pyrazole, optionally substituted oxadiazole, optionally substituted triazole, 4-methylthiazol-5-yl, or oxazol-5-yl group.

In another embodiment, the degradation tag is a moiety of FORMULA 7A:

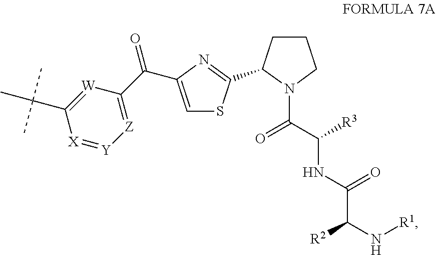

wherein

V, W, X, and Z are independently selected from CR⁴ and N; and

R¹, R², R³, and R⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈alkoxy, optionally substituted C₁-C₈alkylamino, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ hydroxyalkyl, optionally substituted 3-10 membered carbocyclyl, and optionally substituted 4-10 membered heterocyclyl.

In another embodiment, the degradation tag is a moiety of FORMULA 7B:

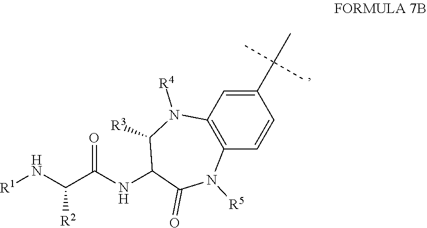

wherein

R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;

R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl, wherein

R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl, or

R⁶ and R⁷ together with the atom to which they are connected form a 4-8 membered cycloalkyl or heterocyclyl ring.

In another embodiment, the degradation tag is derived from any of the following:

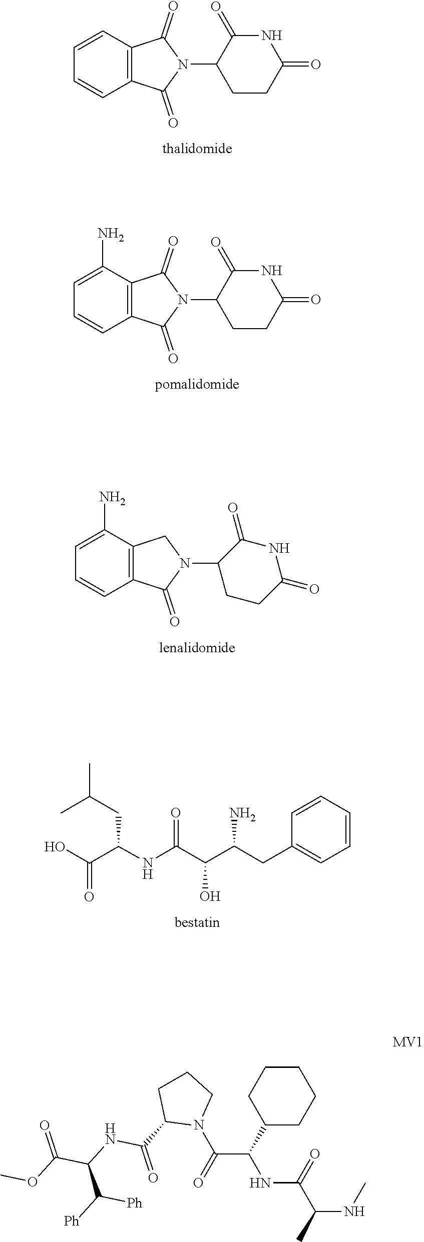

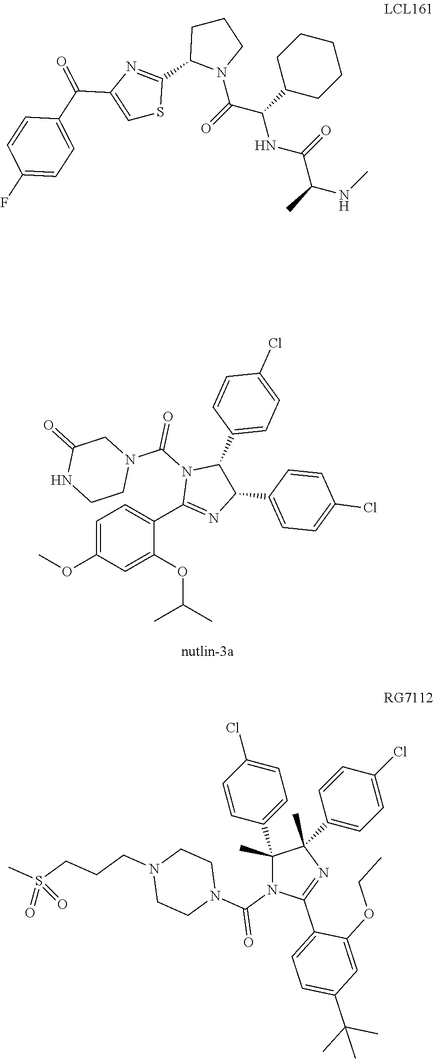

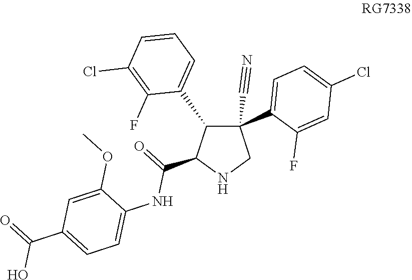 [SMILES: COc1cc(C(=O)O)ccc1NC(=O)[C@@H]1NC[C@](C#N)(c2ccc(Cl)cc2F)[C@H]1c1cccc(Cl)c1F]

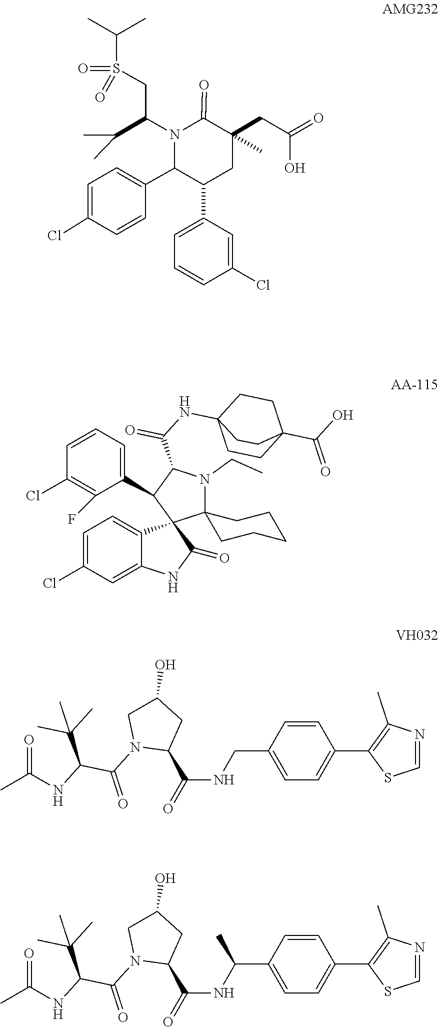

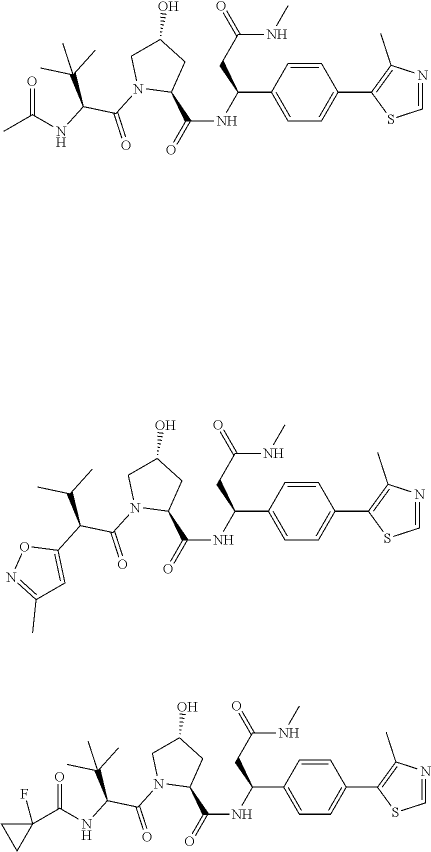 [SMILES: CNC(=O)C[C@H](NC(=O)[C@@H]1C[C@@H](O)CN1C(=O)[C@@H](NC(C)=O)C(C)(C)C)c1ccc(-c2scnc2C)cc1 ; CNC(=O)C[C@H](NC(=O)[C@@H]1C[C@@H](O)CN1C(=O)[C@@H](c1cc(C)no1)C(C)C)c1ccc(-c2scnc2C)cc1 ; CNC(=O)C[C@H](NC(=O)[C@@H]1C[C@@H](O)CN1C(=O)[C@@H](NC(=O)C1(F)CC1)C(C)(C)C)c1ccc(-c2scnc2C)cc1]

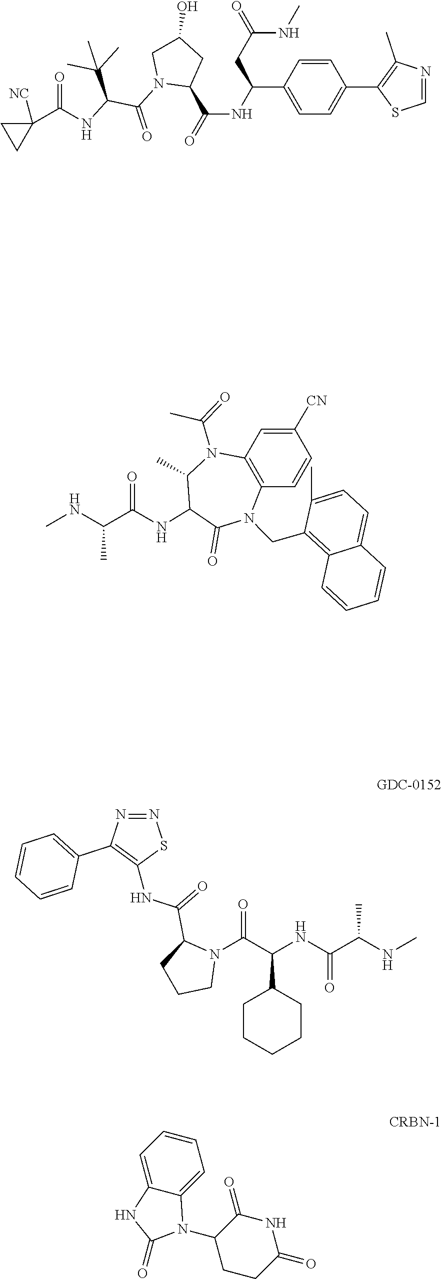 [SMILES: CNC(=O)C[C@H](NC(=O)[C@@H]1C[C@@H](O)CN1C(=O)[C@@H](NC(=O)C1(C#N)CC1)C(C)(C)C)c1ccc(-c2scnc2C)cc1 ; CN[C@@H](C)C(=O)NC1C(=O)N(Cc2c(C)ccc3ccccc23)c2ccc(C#N)cc2N(C(C)=O)[C@H]1C ; CN[C@@H](C)C(=O)N[C@H](C(=O)N1CCC[C@H]1C(=O)Nc1snnc1-c1ccccc1)C1CCCCC1 ; O=C1CCC(n2c(=O)[nH]c3ccccc32)C(=O)N1]

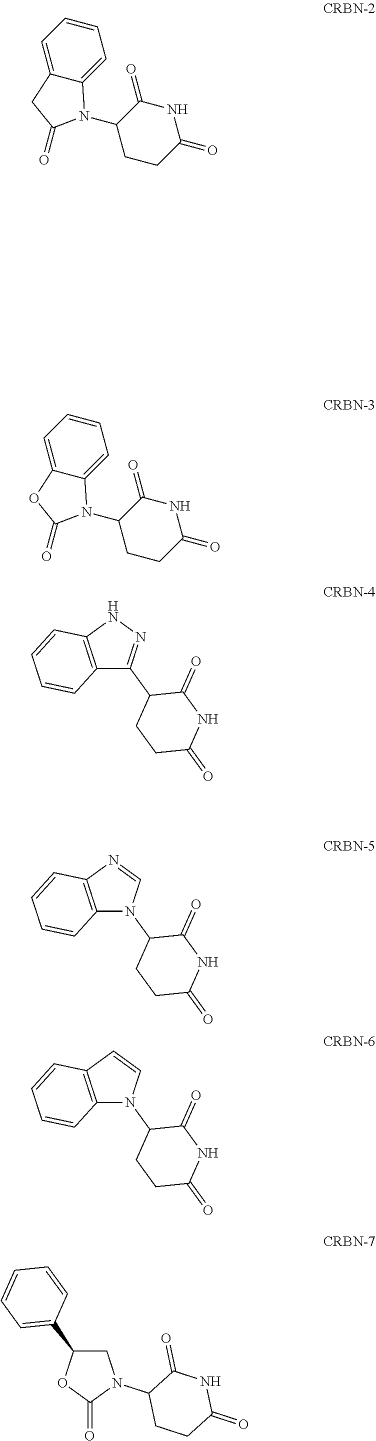 [SMILES: O=C1CCC(N2C(=O)Cc3ccccc32)C(=O)N1 ; O=C1CCC(n2c(=O)oc3ccccc32)C(=O)N1 ; O=C1CCC(c2n[nH]c3ccccc23)C(=O)N1 ; O=C1CCC(n2cnc3ccccc32)C(=O)N1 ; O=C1CCC(n2ccc3ccccc32)C(=O)N1 ; O=C1CCC(N2C[C@H](c3ccccc3)OC2=O)C(=O)N1]

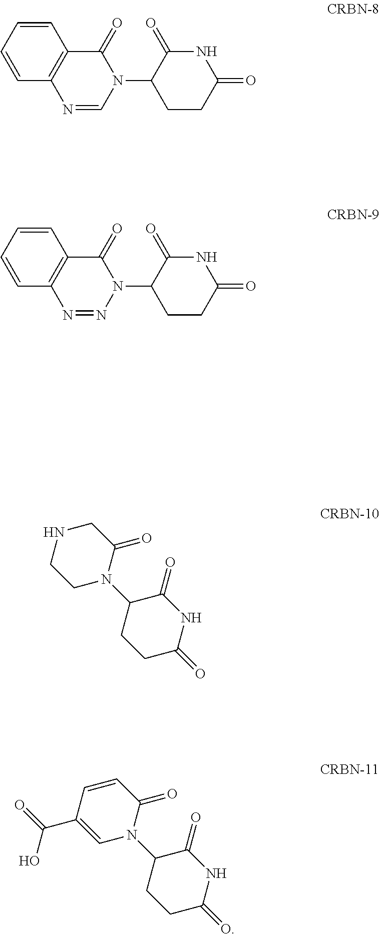 [SMILES: O=C1CCC(n2cnc3ccccc3c2=O)C(=O)N1 ; O=C1CCC(n2nnc3ccccc3c2=O)C(=O)N1 ; O=C1CCC(N2CCNCC2=O)C(=O)N1 ; O=C1CCC(n2cc(C(=O)O)ccc2=O)C(=O)N1]

In another embodiment, the degradation tag is derived from any of the following: thalidomide, pomalidomide, lenalidomide, CRBN-1, CRBN-2, CRBN-3, CRBN-4, CRBN-5, CRBN-6, CRBN-7, CRBN-8, CRBN-9, CRBN-10, and CRBN-11.

In another embodiment, the degradation tag is selected from the group consisting of:

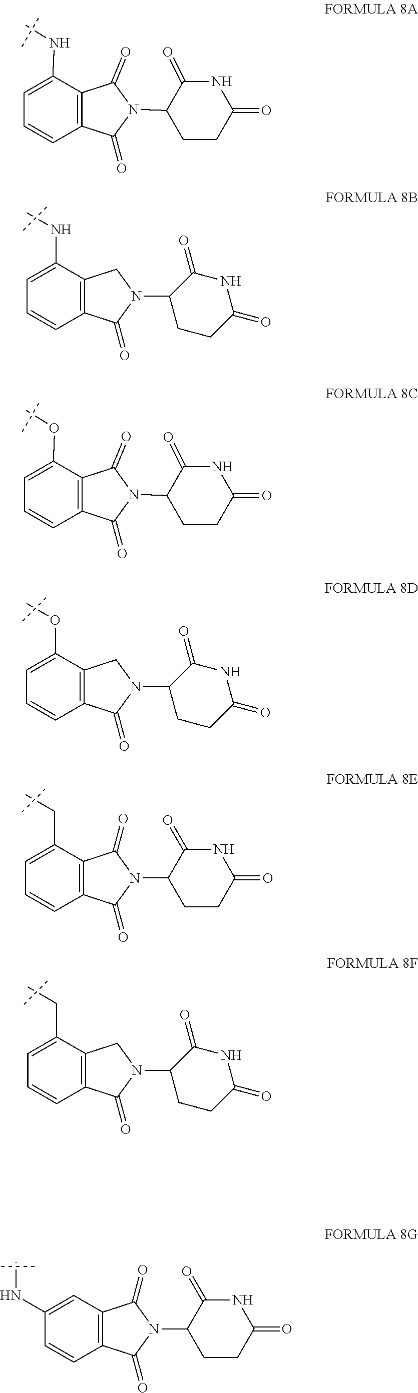

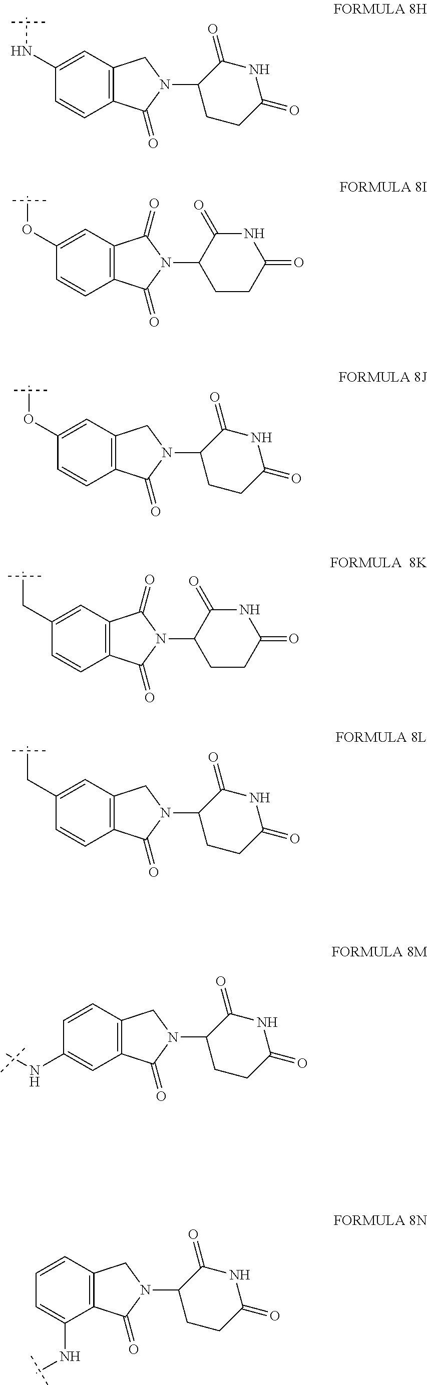

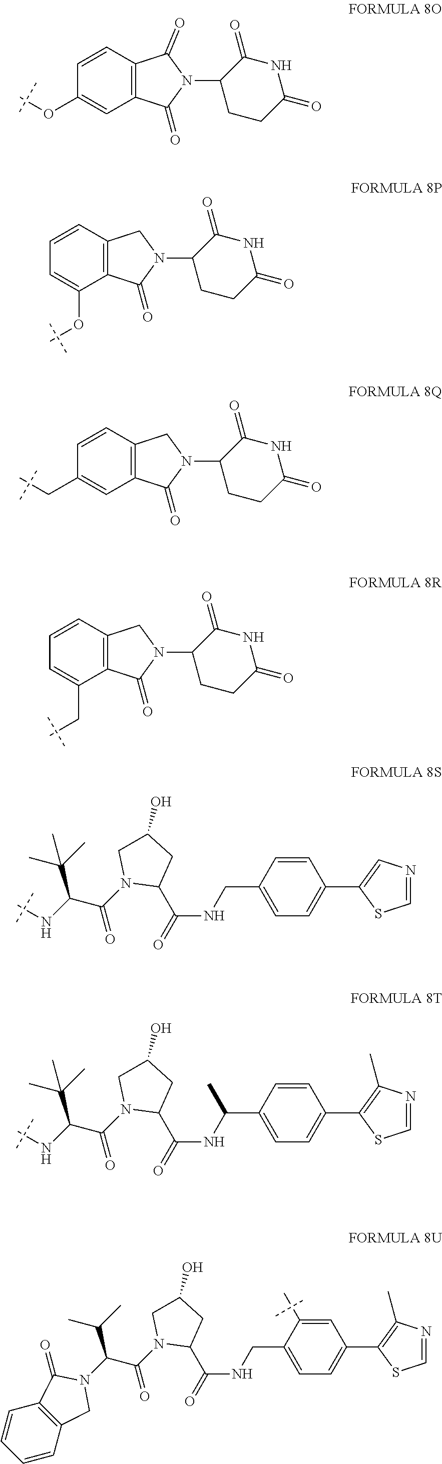

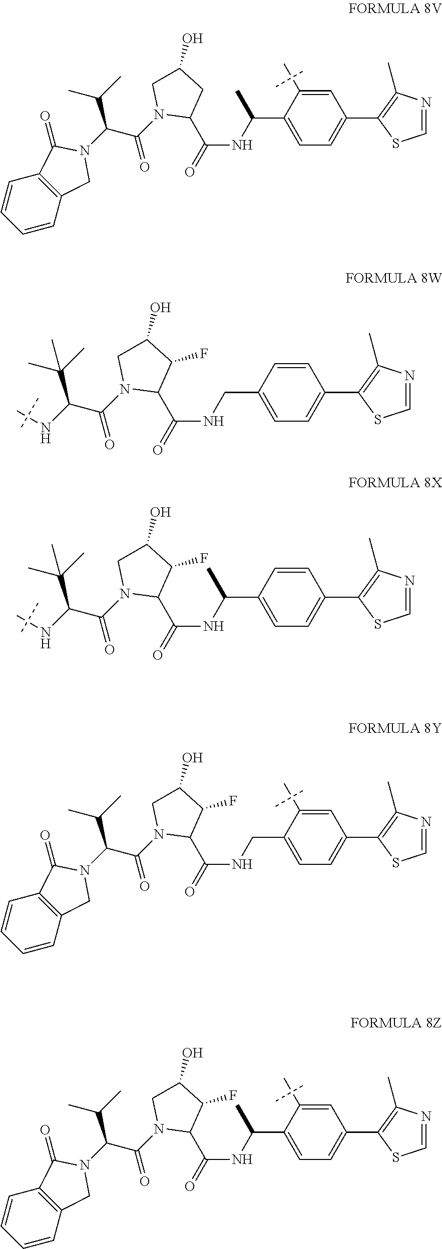

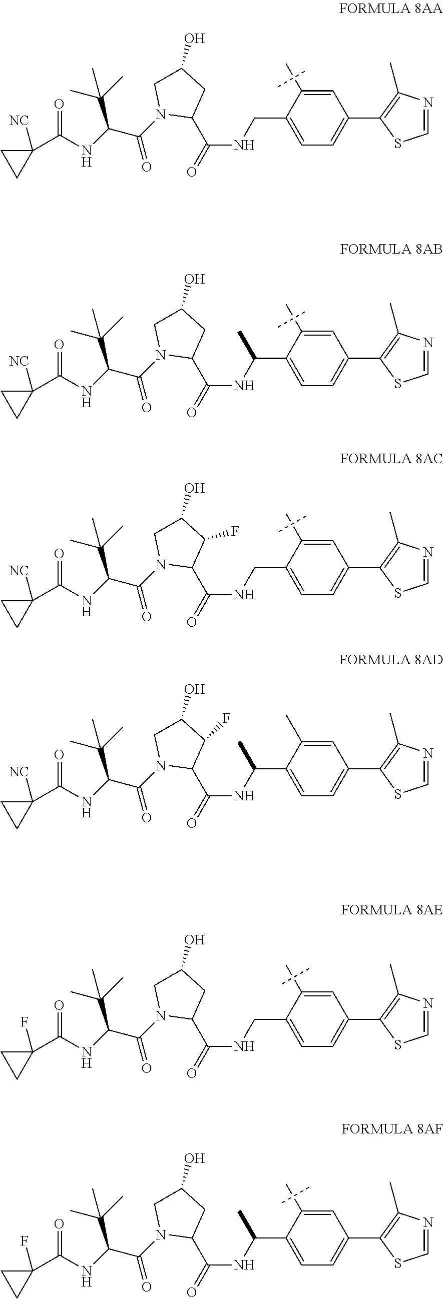

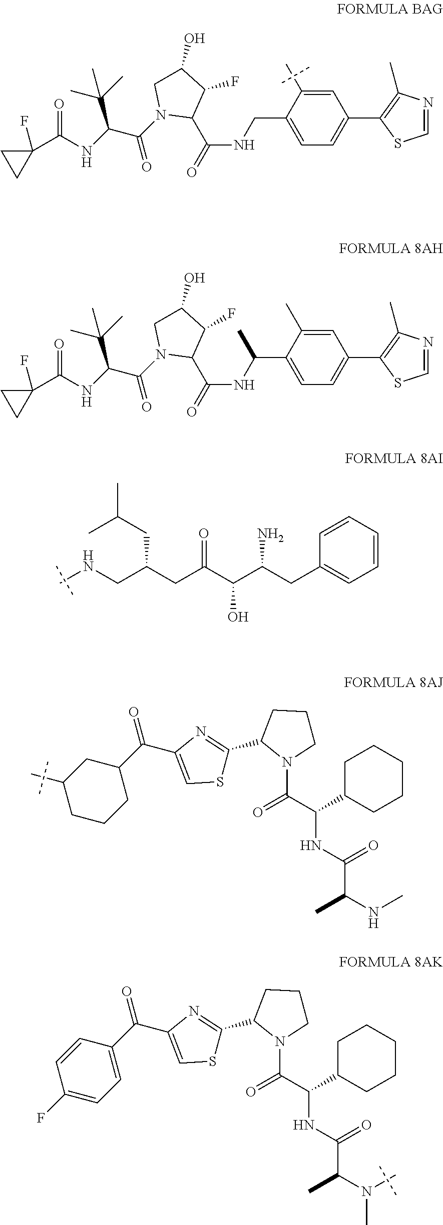

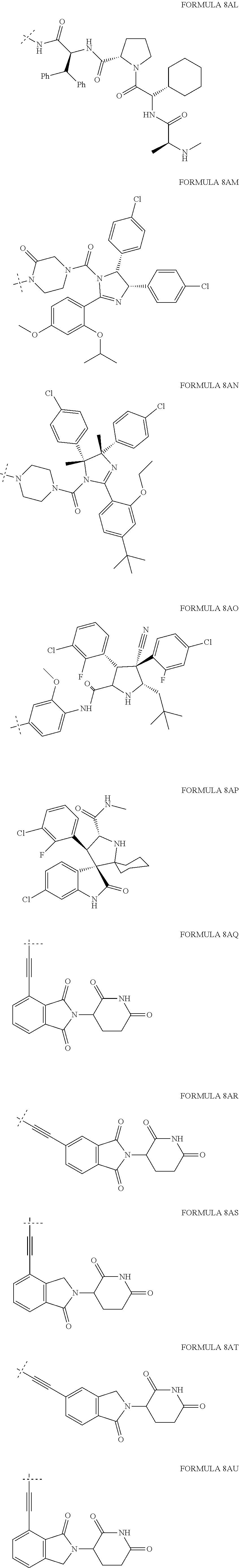

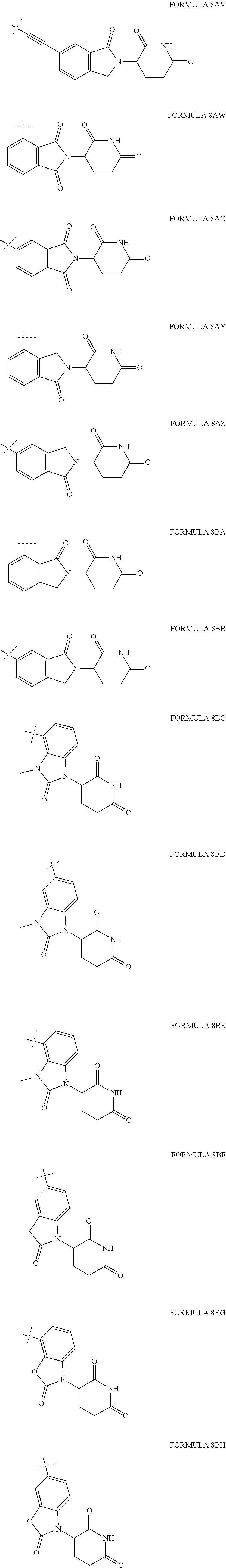

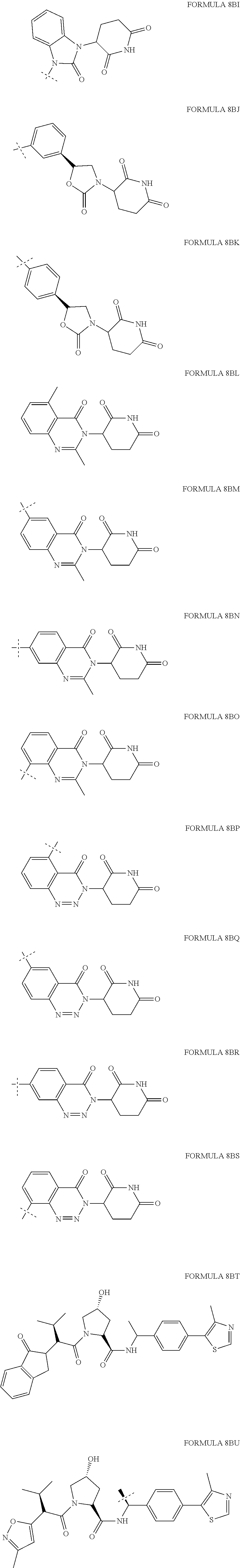

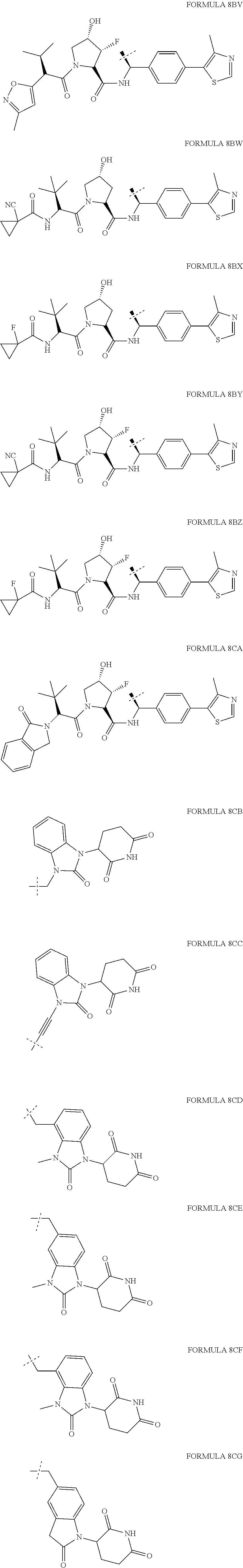

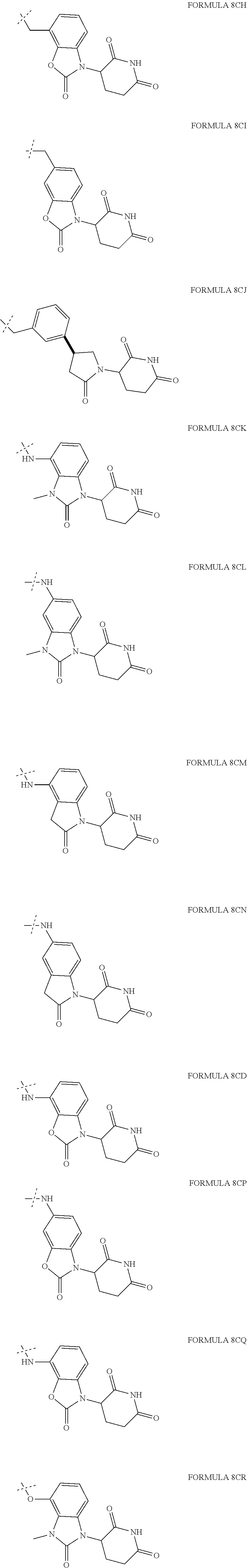

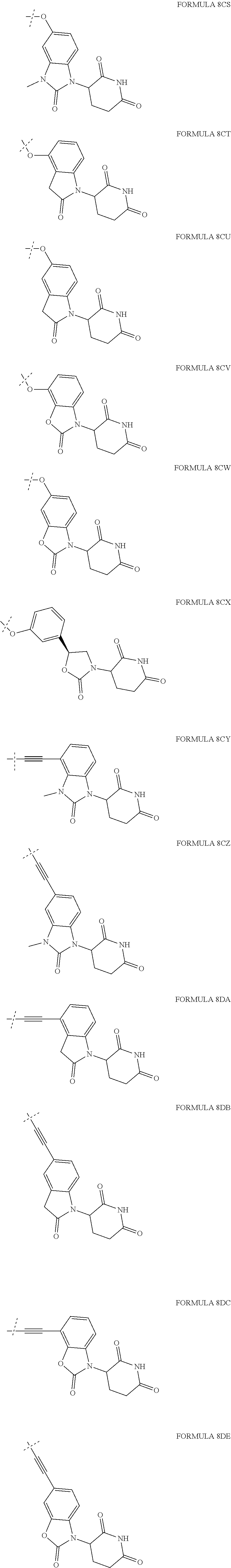

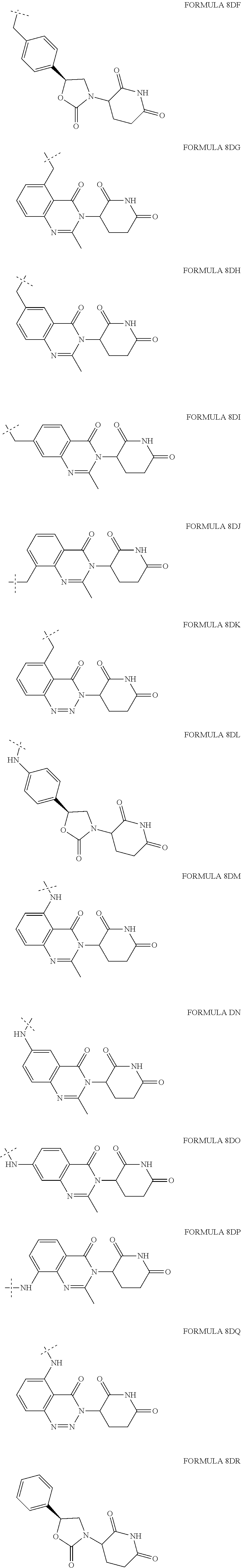

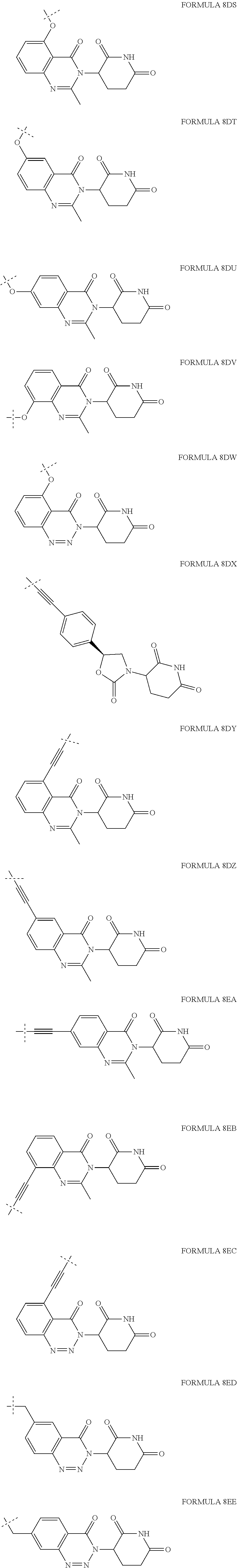

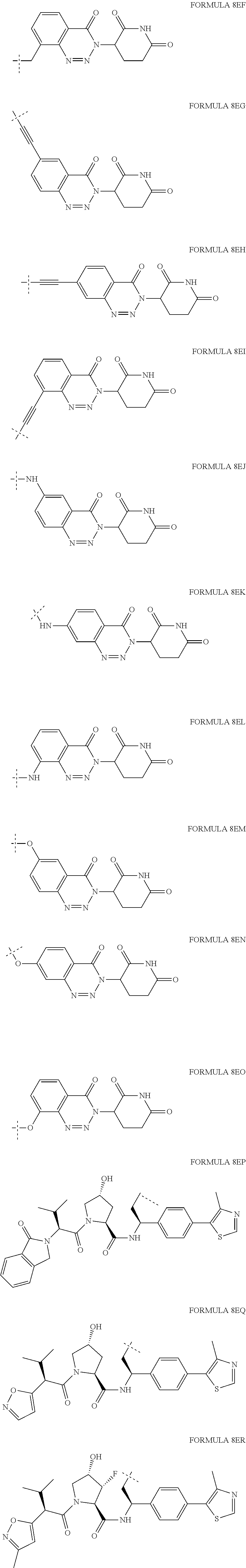

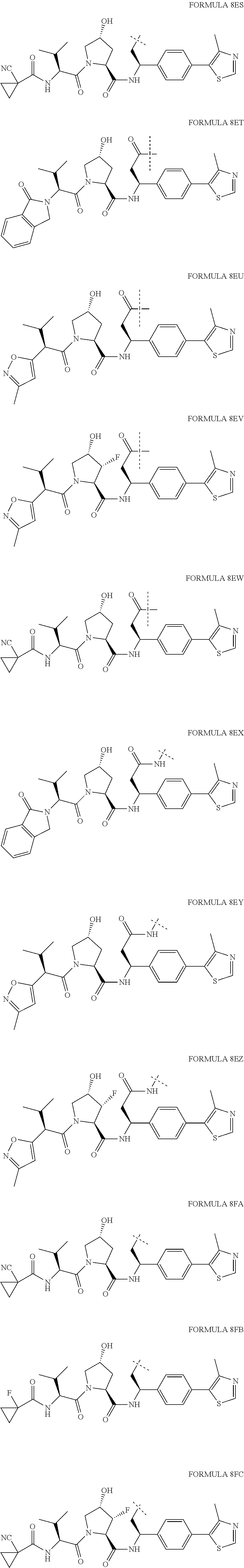

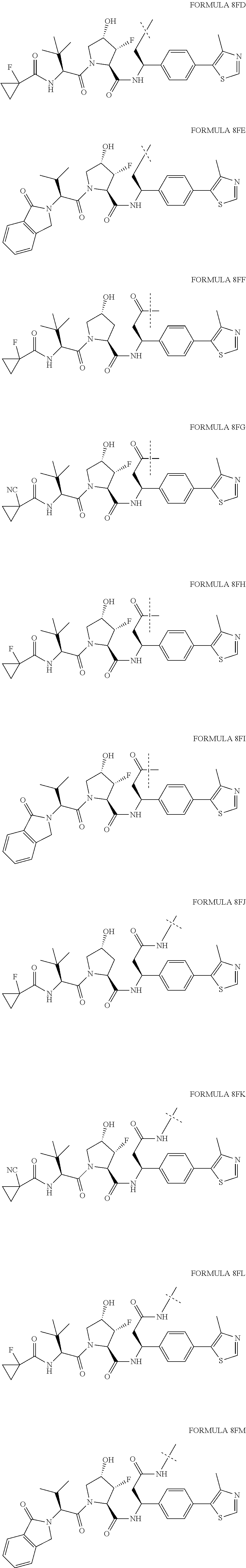

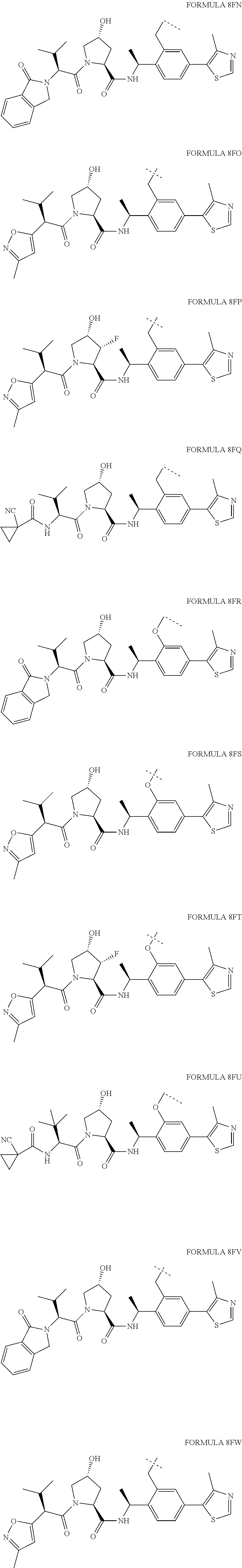

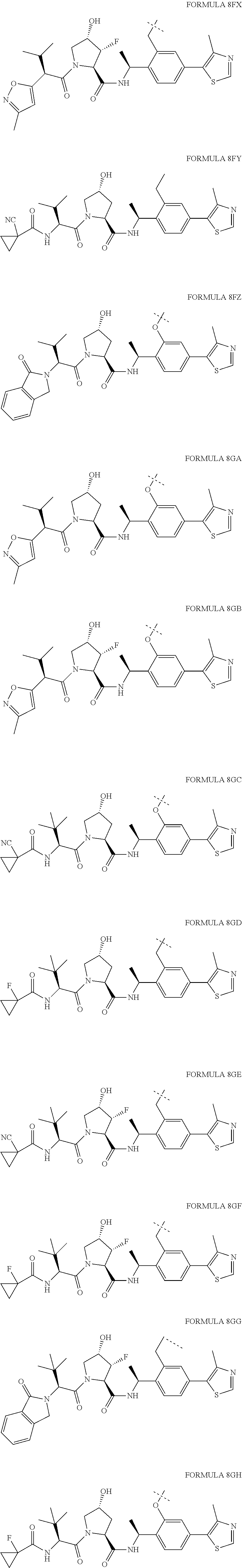

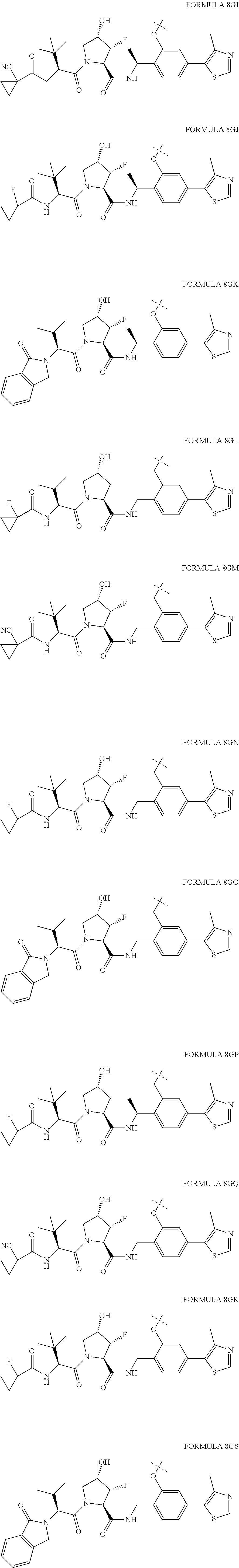

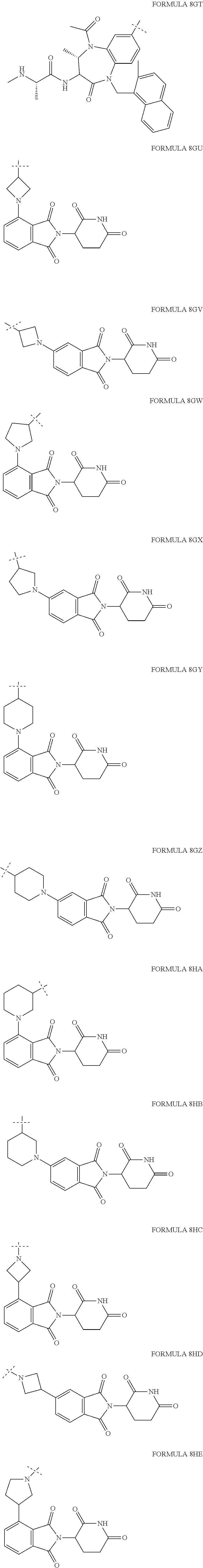

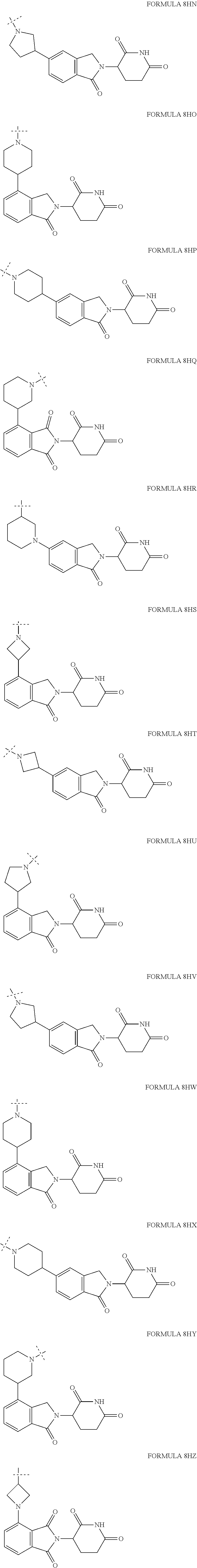

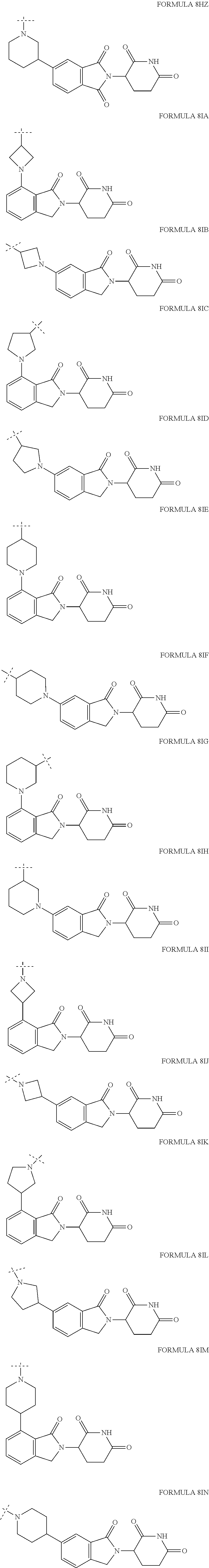

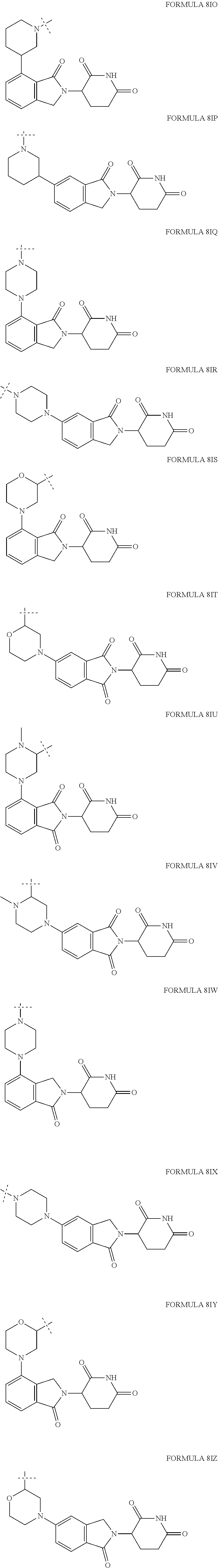

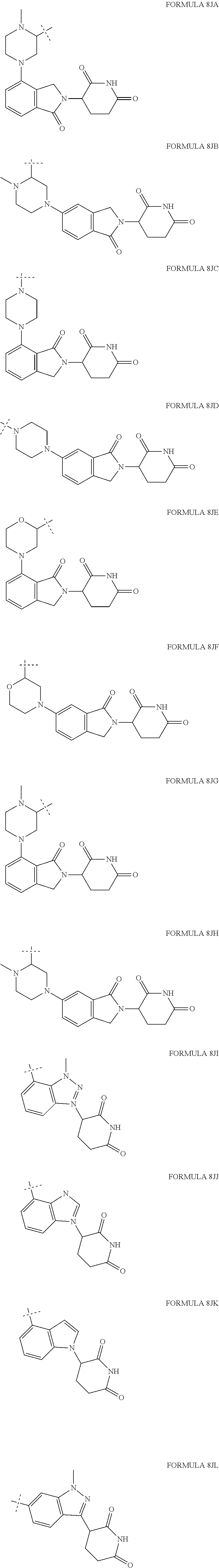

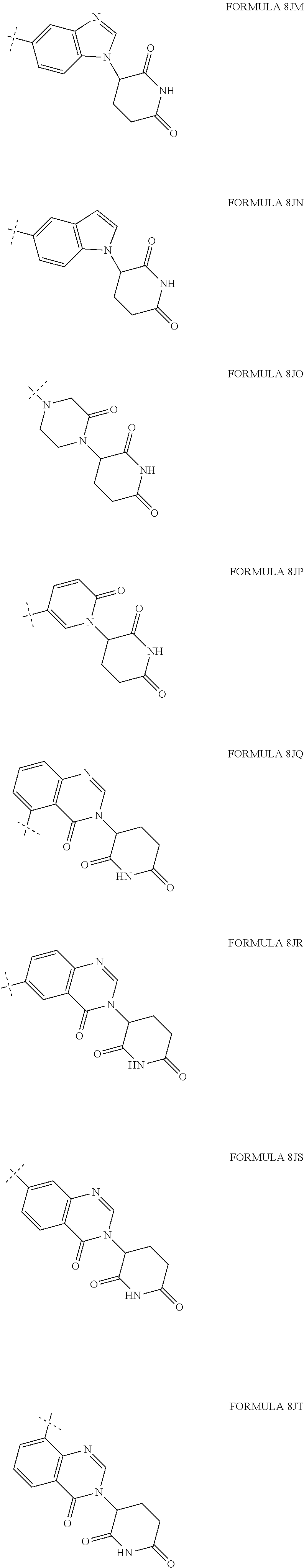

Linker Moiety

As used herein, a “linker” or “linker moiety” is a bond, molecule, or group of molecules that binds two separate entities to one another. Linkers provide for optimal spacing of the two entities. The term “linker” in some aspects refers to any agent or molecule that bridges the JAK ligand to the degradation tag. One of ordinary skill in the art recognizes that sites on the JAK ligand or the degradation tag, which are not necessary for the function of the bifunctional degraders of the present disclosure, are ideal sites for attaching a linker, provided that the linker, once attached to the conjugate of the present disclosures, does not interfere with the function of the JAK ligand, i.e., its ability to bind JAK, or the function of the degradation tag, i.e., its ability to recruit a ubiquitin ligase.

The length of the linker of the heterobifunctional compound can be adjusted to minimize the molecular weight of the heterobifunctional compounds, avoid the clash of the JAK ligand or targeting moiety with the ubiquitin ligase and/or induce JAK misfolding by the hydrophobic tag. In certain embodiments, the linker comprises acyclic or cyclic saturated or unsaturated carbon, ethylene glycol, amide, amino, ether, urea, carbamate, aromatic, heteroaromatic, heterocyclic or carbonyl groups. In certain embodiments, the length of the linker is 0, 1, 2, 3, 4, 5, 6, 7, 8, 9, 10, 11, 12, 13, 14, 15, 16, 17, 18, 19, 20 or more atoms.

In some embodiments, the linker moiety is of FORMULA 9:

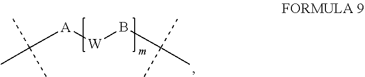

wherein

A, W and B, at each occurrence, are independently selected from null, or bivalent moiety selected from R′—R″, R′COR″, R′CO₂R″, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R′)R″, R′N(R¹)R″, R′N(R¹)COR″, R′NR¹C(O)OR″, R′NR¹CON(R²)R″, R′NR¹C(S)R″, R′NR¹S(O)R″, R′NR¹S(O)₂R″, and R′NR¹S(O)₂N(R²)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R¹ and R², R′ and R¹, R′ and R², R″ and R¹, R″ and R² together with the atom to which they are connected form a 3-20 membered cycloalkyl or 4-20 membered heterocyclyl ring; and

m is 0 to 15.

In some embodiments, the linker moiety is of FORMULA 9:

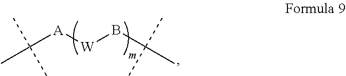

wherein

A, W and B, at each occurrence, are independently selected from null, or bivalent moiety selected from R′—R″, R′COR″, R′CO₂R″, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R′)R″, R′N(R¹)R″, R′N(R¹)COR″, R′N(R¹)C(O)OR″, R′N(R¹)CON(R²)R″, R′N(R¹)C(S)R″, R′N(R¹)S(O)R″, R′N(R¹)S(O)₂R″, R′N(R¹)S(O)₂N(R²)R″, wherein

R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R¹ and R², R′ and R¹, R′ and R², R″ and R¹, R″ and R² together with the atom to which they are connected form a 3-20 membered cycloalkyl or 4-20 membered heterocyclyl ring; and

m is 0 to 15.

In one embodiment, the linker moiety is of FORMULA 9A:

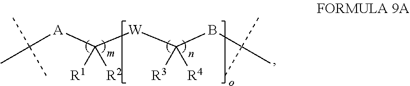

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W and B, at each occurrence, are independently selected from null, or bivalent moiety selected from R′—R″, R′COR″, R′CO₂R″, R′C(O)N(R⁵)R″, R′C(S)N(R⁵)R″, R′OR″, R′OC(O)R″, R′OC(O)OR″, R′OCON(R⁵)R″, R′SR″, R′SOR″, R′SO₂R″, R′SO₂N(R⁵)R″, R′N(R⁵)R″, R′N(R⁵)COR″, R′N(R⁵)C(O)OR″, R′N(R⁵)CON(R⁶)R″², R′N(R⁵)C(S)R″, R′N(R⁵)S(O)R″, R′N(R⁵)S(O)₂R″, and R′N(R⁵)S(O)₂N(R⁶)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;

m is 0 to 15;

n, at each occurrence, is 0 to 15; and

o is 0 to 15.

In another embodiment, the linker moiety is of FORMULA 9A:

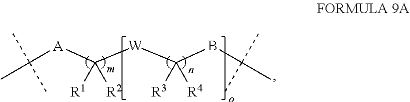

wherein

R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or

R¹ and R², R³ and R⁴ together with the atom to which they are connected form a 3-20 membered cycloalkyl or 4-20 membered heterocyclyl ring;

A, W and B, at each occurrence, are independently selected from null, or bivalent moiety selected from R′—R″, R′COR″, R′CO₂R″, R′C(O)N(R⁵)R″, R′C(S)N(R⁵)R″, R′OR″, R′OC(O)R″, R′OC(O)OR″, R′OCON(R⁵)R″, R′SR″, R′SOR″, R′SO₂R″, R′SO₂N(R⁵)R″, R′N(R⁵)R″, R′N(R⁵)COR″, R′N(R⁵)C(O)OR″, R′N(R⁵)CON(R⁶)R″², R′N(R⁵)C(S)R″, R′N(R⁵)S(O)R″, R′N(R⁵)S(O)₂R″, and R′N(R⁵)S(O)₂N(R⁶)R″, wherein

R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;

m is 0 to 15;

n, at each occurrence, is 0 to 15; and

o is 0 to 15.

In another embodiment, the linker moiety is of FORMULA 9B:

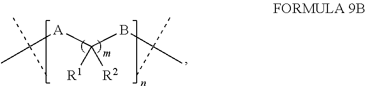

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or

R¹ and R² together with the atom to which they are connected form a 3-20 membered cycloalkyl or 4-20 membered heterocyclyl ring;

A and B, at each occurrence, are independently selected from null, or bivalent moiety selected from R′—R″, R′COR″, R′CO₂R″, R′C(O)N(R³)R″, R′C(S)N(R³)R″, R′OR″, R′OC(O)R″, R′OC(O)OR″, R′OCON(R³)R″, R′SR″, R′SOR″, R′SO₂R″, R′SO₂N(R³)R″, R′N(R³)R″, R′N(R³)COR″, R′N(R³)C(O)OR″, R′N(R³)CON(R⁴)R″, R′N(R³)C(S)R″, R′N(R³)S(O)R″, R′N(R³)S(O)₂R″, R′N(R³)S(O)₂N(R⁴)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;

each m is 0 to 15; and

n is 0 to 15.

In another embodiment, the linker moiety is of FORMULA 9B:

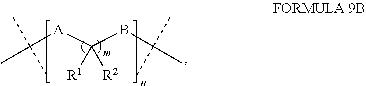

wherein

R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or

R¹ and R² together with the atom to which they are connected form a 3-20 membered cycloalkyl or 4-20 membered heterocyclyl ring;

A and B, at each occurrence, are independently selected from null, or bivalent moiety selected from R′—R″, R′COR″, R′CO₂R″, R′C(O)N(R³)R″, R′C(S)N(R³)R″, R′OR″, R′OC(O)R″, R′OC(O)OR″, R′OCON(R³)R″, R′SR″, R′SOR″, R′SO₂R″, R′SO₂N(R³)R″, R′N(R³)R″, R′N(R³)COR″, R′N(R³)C(O)OR″, R′N(R³)CON(R⁴)R″, R′N(R³)C(S)R″, R′N(R³)S(O)R″, R′N(R³)S(O)₂R″, and R′N(R³)S(O)₂N(R⁴)R″ wherein

R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R³ and R⁴, R¹ and R³, R¹ and R⁴, R″ and R³, R″ and R⁴ together with the atom to which they are connected form a 3-20 membered cycloalkyl or 4-20 membered heterocyclyl ring;

each m is 0 to 15; and

n is 0 to 15.

In another embodiment, the linker moiety is of FORMULA 9C:

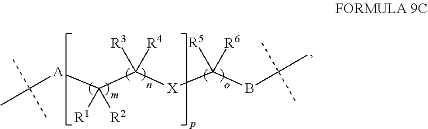

wherein

X is selected from O, NH, and NR⁷;

R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;

A and B are independently selected from null, or bivalent moiety selected from R′—R″, R′COR″, R′CO₂R″, R′C(O)NR″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R′)R″, R′N(R⁸)R″, R′NR⁸COR″, R′NR⁸C(O)OR″, R′NR⁸CON(R⁹)R″, R′NR⁸C(S)R″, R′NR⁸S(O)R″, R′NR⁸S(O)₂R″, R′NR⁸S(O)₂N(R⁹)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein

R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;

m, at each occurrence, is 0 to 15;

n, at each occurrence, is 0 to 15;

o is 0 to 15; and

p is 0 to 15.

In another embodiment, the linker moiety is of FORMULA 9C:

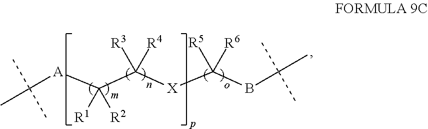

wherein

X is selected from O, NH, and NR⁷;

R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;

A and B are independently selected from null, or bivalent moiety selected from R′—R″, R′COR″, R′CO₂R″, R′C(O)NR″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R′)R″, R′N(R⁸)R″, R′NR⁸COR″, R′NR⁸C(O)OR″, R′NR⁸CON(R⁹)R″, R′NR⁸C(S)R″, R′NR⁸S(O)R″, R′NR⁸S(O)₂R″, R′NR⁸S(O)₂N(R⁹)R″, wherein

R′ and R″ are independently selected from null, or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;

R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or

R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;

m, at each occurrence, is 0 to 15;

n, at each occurrence, is 0 to 15;

o is 0 to 15; and

p is 0 to 15.

In one refinement, A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH— CH₂—NH—CH₂, CH₂—NH—CH₂.

In another refinement, o is 0 to 5.

In another refinement, the linker moiety comprises a ring selected from the group consisting of a 3 to 13 membered ring, a 3 to 13 membered fused ring, a 3 to 13 membered bridged ring, and a 3 to 13 membered spiro ring.

In another embodiment, the linker moiety comprises one or more rings selected from the group consisting of FORMULAE C1a, C2a, C3a, C4a and C5a

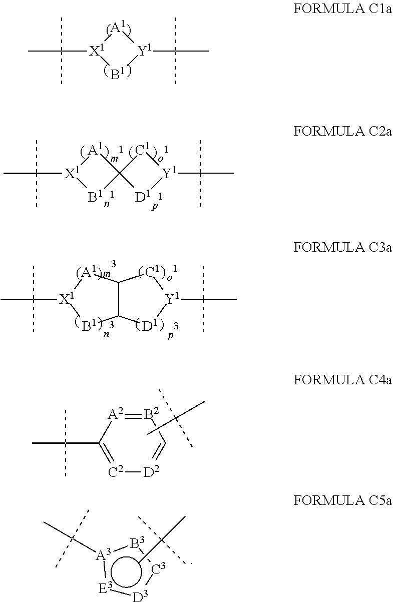

wherein

X′ and Y′ are independently selected from N, and CR^{b};

A¹, B¹, C¹ and D¹, at each occurrence, are independently selected from null, O, CO, SO, SO₂, NR^{b}, and CR^{b}R^{c};

A², B², C², and D², at each occurrence, are independently selected from N, and CR^{b};

A³, B³, C³, D³, and E³, at each occurrence, are independently selected from N, O, S, NR^{b}, and CR^{b};

R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and

m¹, n¹, o¹ and p¹ are independently selected from 0, 1, 2, 3, 4 and 5.

In another refinement, the linker moiety comprises one or more rings selected from the group consisting of formulae C1, C2, C3, C4 and C5:

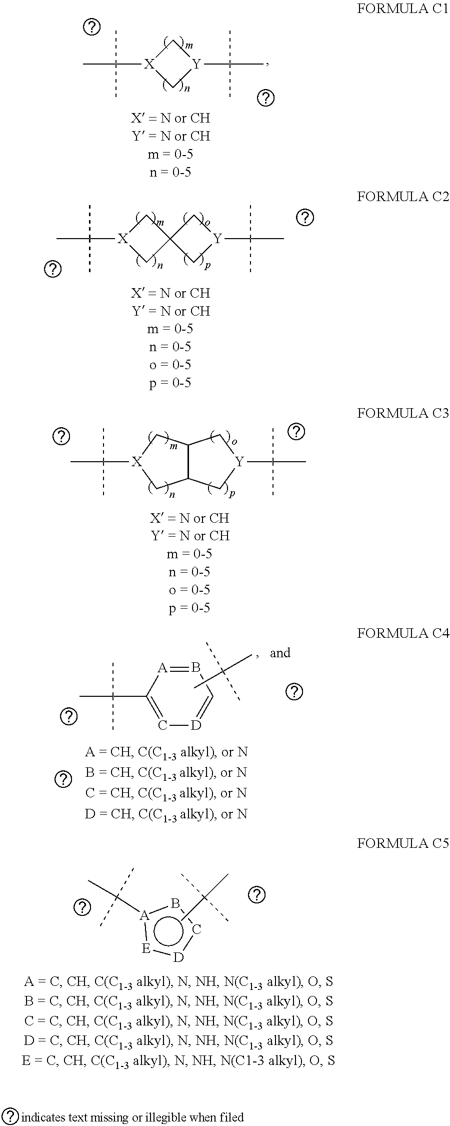

In another refinement, the linker moiety comprises one or more rings selected from Group R, and Group R consists of:

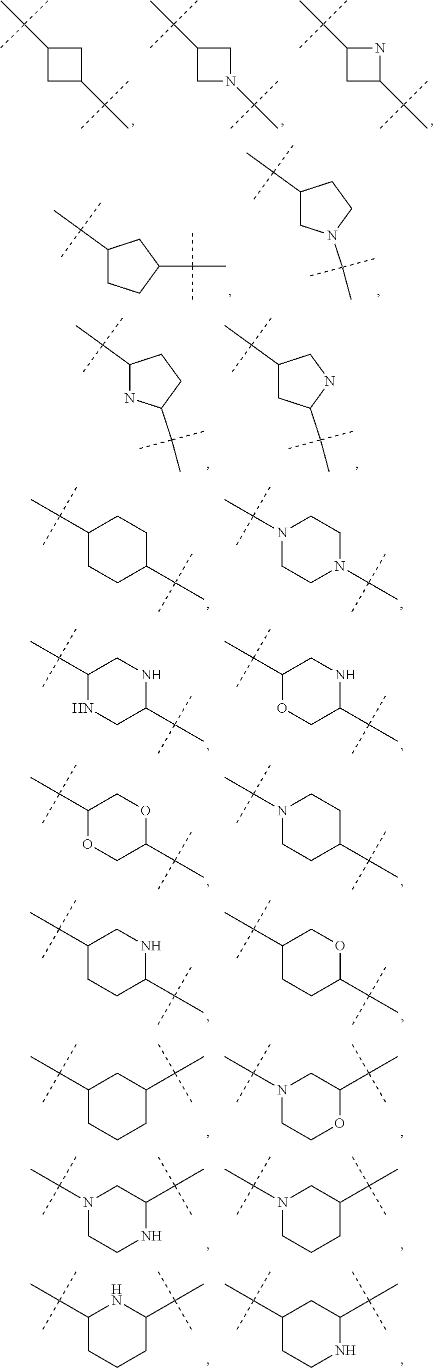

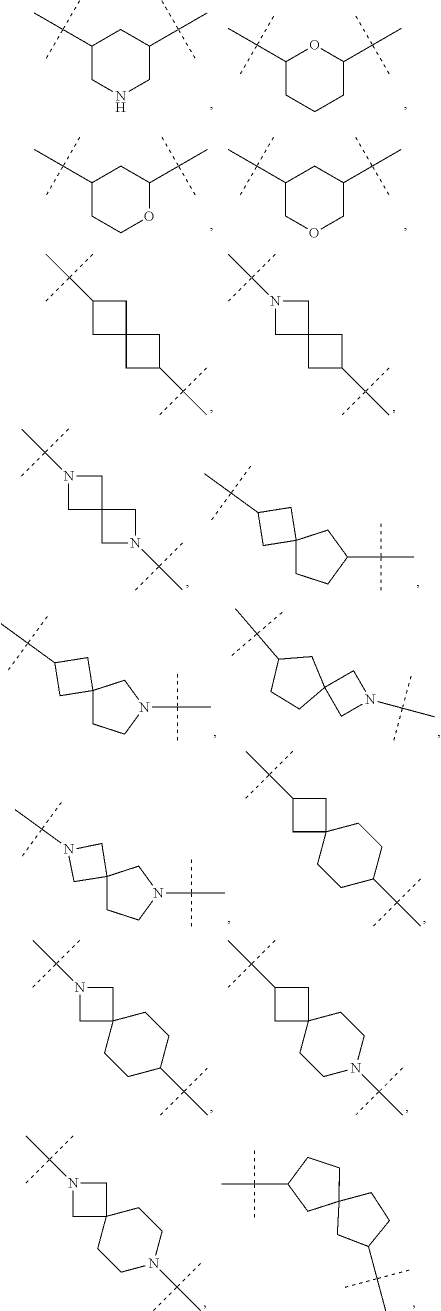

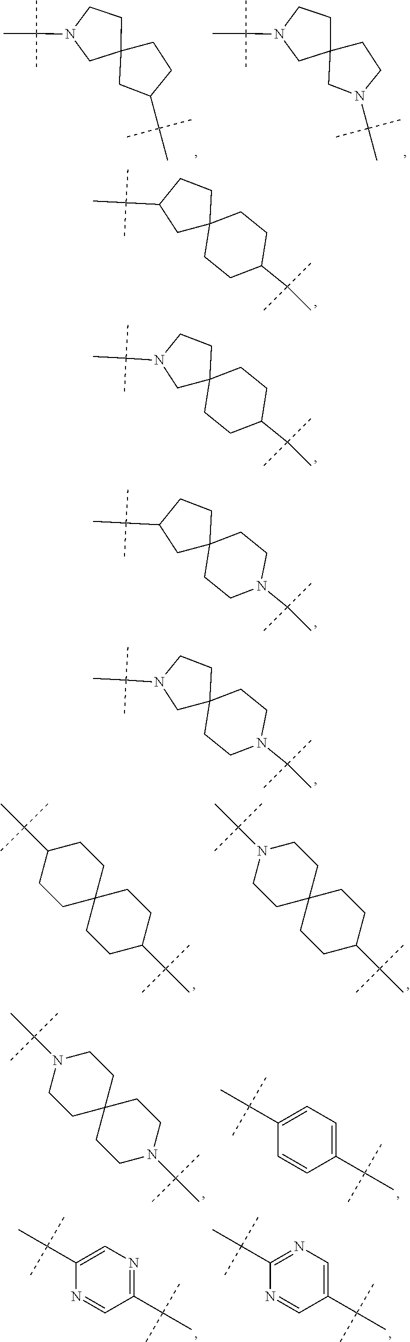

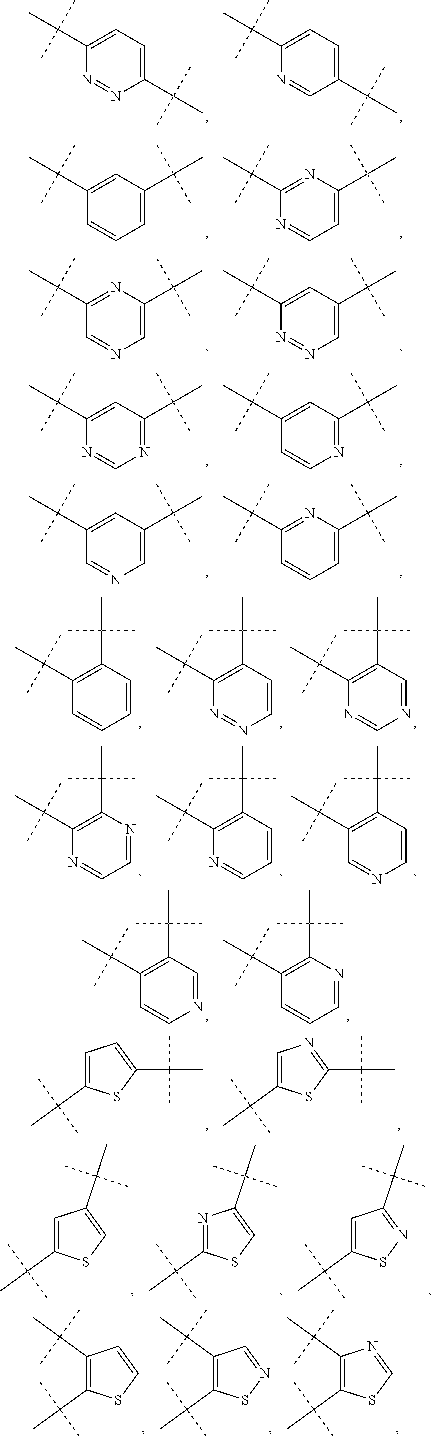

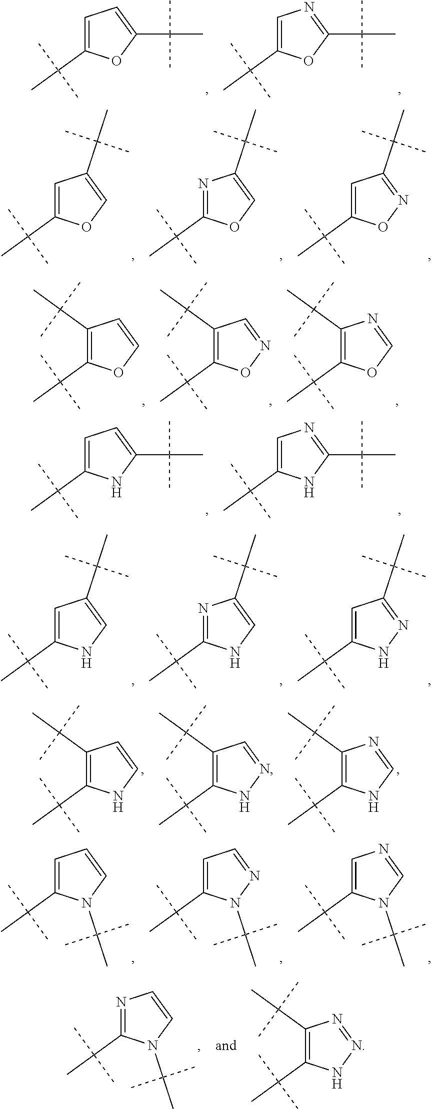

In another embodiment, R^{r} is selected from FORMULA C1a, C2a, C3a, C4a, C5a, C1, C2, C3, C4, and C5 as defined above.

In another embodiment, R is selected from Group R.

In another refinement, the length of the linker is 0 to 40 chain atoms.

In another refinement, the length of the linker is 1 to 20 chain atoms.

In another refinement, the length of the linker is 2 to 12 chain atoms.

In another refinement, the linker is selected from —(CO)—(CH₂)₁₋₈—, —(CH₂)₁₋₉—, —(CH₂)₁₋₂—(CO)—NH—(CH₂)₂₋₉—, —(CH₂)₁₋₂—(CO)—NH—(CH₂)₁₋₃—(OCH₂CH₂)₁₋₇, and —(CH₂)₀₋₁—(CO)—(CH₂)₁₋₃—(OCH₂CH₂)₁₋₇.

In another refinement, the linker is —(CO)—(CH₂)₁₋₈—, —(CH₂)₁₋₉—, —(CH₂)₁₋₂(CO)—NH—(CH₂)₂₋₉—, or —(CH₂)₁₋₂—(CO)—NH—(CH₂)₁₋₃—(OCH₂CH₂)₁₋₇.

In another refinement, (preferably when the degradation tag is a moiety of FORMULA 6) the linker is —(CO)—(CH₂)₁₋₁₀—(CO)—, or —(CO)—(CH₂)₁₋₉—(CO)—.

In another refinement, (preferably when the degradation tag is a moiety of FORMULA 6) the linker is —(CO)—(CH₂)₁₋₃—O—(CH₂CH₂O)₀₋₅—(CH₂)₁₋₃—(CO)—, or —(CO)—(CH₂)—O—(CH₂CH₂O)₀₋₄—(CH₂)—(CO)—, or —(CO)—(CH₂)₂—O—(CH₂CH₂O)₀₋₄—(CH₂)₂—(CO)—.

In another refinement, (preferably when the degradation tag is a moiety of FORMULA 6) the linker is —(CH₂)₀₋₃—(CO)—NH—(CH₂)₁₋₁₀—(CO)—, —(CH₂)₁₋₂—(CO)—NH—(CH₂)₁₋₁₀—(CO)—, or —(CH₂)—(CO)—NH—(CH₂)₁₋₁₀—(CO)—.

In another refinement, (preferably when the degradation tag is a moiety of FORMULA 6) the linker is —(CH₂)₀₋₃—(CO)—NH—(CH₂)₂₋₃—O—(CH₂CH₂O)₀₋₅—(CH₂)₁₋₃—(CO)—, —(CH₂)₁₋₂—(CO)—NH—(CH₂)₂₋₃—O—(CH₂CH₂O)₀₋₅—(CH₂)₁₋₃—(CO)—, or —(CH₂)—(CO)—NH—(CH₂)₂—O—(CH₂CH₂O)₀₋₄—(CH₂)₁₋₂—(CO)—.

In another refinement, (preferably when the degradation tag selected from FORMULAE 5A, 5B, 5C, 5D, 5E, and 5F) the linker is —(CO)—(CH₂)₁₋₈—, —(CO)—(CH₂)₁₋₇—, —(CH₂)₁₋₉—, or —(CH₂)₁₋₈—.

In another refinement, (preferably when the degradation tag selected from FORMULAE 5A, 5B, 5C, 5D, 5E, and 5F) the linker is —(CH₂)₁₋₂(CO)—NH—(CH₂)₄₋₉—, or —(CH₂)₁₋₂(CO)—NH—(CH₂)₂₋₉—, or —(CH₂)₁₋₂(CO)—NH—(CH₂)₂₋₈—.

In another refinement, (preferably when the degradation tag selected from FORMULAE 5A, 5B, 5C, 5D, 5E, and 5F) the linker is —(CH₂)₁₋₂—(CO)—NH—(CH₂)_{1,2 or 3}—(OCH₂CH₂)₁₋₇—, or —(CH₂)₁₋₂—(CO)—NH—(CH₂)_{1,2 or 3}—(OCH₂CH₂)₅₋₇—, or —(CH₂)₁₋₂—(CO)—NH—(CH₂)_{1,2 or 3}—(OCH₂CH₂)₁₋₅—.

In another refinement, (preferably when the degradation tag selected from FORMULAE 5A, 5B, 5C, 5D, 5E, and 5F) the linker is —(CH₂)₀₋₁—(CO)—(CH₂)_{1,2 or 3}—(OCH₂CH₂)₁₋₇—, —(CH₂)₀₋₁—(CO)—(CH₂)_{1,2 or 3}—(OCH₂CH₂)₅₋₇—, —(CH₂)₀₋₁—(CO)—(CH₂)_{1,2 or 3}—(OCH₂CH₂)₁₋₅—.

In another refinement, when the JAK ligand derived from NVP-BSK805 (and preferably the degradation tag is selected from FORMULAE 5A, 5B, 5C, 5D, 5E, and 5F), the linker is —(CO)—(CH₂)₃₋₈-(preferably, —(CO)—(CH₂)₄₋₇—; more preferably, —(CO)—(CH₂)₄₋₅—).

In another refinement, when the JAK ligand is FORMULA 3A (and preferably the degradation tag is selected from FORMULAE 5A, 5B, 5C, 5D, 5E, and 5F), the linker is —(CO)—(CH₂)₃₋₈-(preferably, —(CO)—(CH₂)₄₋₇—; more preferably, —(CO)—(CH₂)₄₋₅—).

In another refinement, when the JAK ligand derived from Cmpd 12 (and preferably the degradation tag is selected from FORMULAE 5A, 5B, 5C, 5D, 5E, and 5F), the linker is —(CO)—(CH₂)₃₋₈-(preferably, —(CO)—(CH₂)₄₋₇—; more preferably, —(CO)—(CH₂)₅₋₇—); or the linker is —(CH₂)₁₋₂—(CO)—NH—(CH₂)₅₋₉— (preferably, —(CH₂)₁₋₂—(CO)—NH—(CH₂)₇₋₈—).

In another refinement, when the JAK ligand is FORMULA 3C and 3D (and preferably the degradation tag is selected from FORMULAE 5A, 5B, 5C, 5D, 5E, and 5F), the linker is —(CO)—(CH₂)₃₋₈-(preferably, —(CO)—(CH₂)₄₋₇—; more preferably, —(CO)—(CH₂)₅₋₇—); or the linker is —(CH₂)₁₋₂—(CO)—NH—(CH₂)₅₋₉— (preferably, —(CH₂)₁₋₂—(CO)—NH—(CH₂)₇₋₈—).

In another refinement, when the JAK ligand derived from TG101209 (and preferably the degradation tag is selected from FORMULAE 5A, 5B, 5C, 5D, 5E, and 5F), the linker r is —(CO)—(CH₂)₁₋₇-(preferably, —(CO)—(CH₂)₁₋₂— or —(CO)—(CH₂)₆₋₇—); or the linker is —(CH₂)₁₋₂—(CO)—NH—(CH₂)₄₋₇— (preferably, —(CH₂)₁₋₂—(CO)—NH—(CH₂)₅₋₆—); or the linker is —(CH₂)₁₋₂—(CO)—NH—(CH₂)_{1,2 or 3}—(OCH₂CH₂)₁₋₇ (preferably, —(CH₂)₁₋₂—(CO)—NH—(CH₂)_{1,2 or 3}—(OCH₂CH₂)₄₋₇—; more preferably, —(CH₂)₁₋₂—(CO)—NH—(CH₂)_{1,2 or 3}—(OCH₂CH₂)₅—). In another refinement, when the JAK ligand is FORMULA 31 (and preferably the degradation tag is selected from FORMULAE 5A, 5B, 5C, 5D, 5E, and 5F), the linker is —(CO)—(CH₂)₁₋₇— (preferably, —(CO)—(CH₂)₁₋₂— or —(CO)—(CH₂)₆₋₇—); or the linker is —(CH₂)₁₋₂—(CO)—NH—(CH₂)₄₋₇— (preferably, —(CH₂)₁₋₂—(CO)—NH—(CH₂)₅₋₆—); or the linker is —(CH₂)₁₋₂—(CO)—NH—(CH₂)_{1,2 or 3}—(OCH₂CH₂)₁₋₇ (preferably, —(CH₂)₁₋₂—(CO)—NH—(CH₂)_{1,2 or 3}—(OCH₂CH₂)₄₋₇—; more preferably, —(CH₂)₁₋₂—(CO)—NH—(CH₂)_{1,2 or 3}—(OCH₂CH₂)₅—).

Without wishing to be bound by any particular theory, it is contemplated herein that, in some embodiments, attaching pomalidomide or VHL-1 to either portion of the molecule can recruit the cereblon E3 ligase or VHL E3 ligase to JAK.

The heterobifunctional compounds disclosed herein can selectively affect JAK-mediated disease cells compared to WT (wild type) cells (i.e., an heterobifunctional compound able to kill or inhibit the growth of an JAK-mediated disease cell while also having a relatively low ability to lyse or inhibit the growth of a WT cell), e.g., possess a GI₅₀ for one or more JAK-mediated disease cells more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than its GI₅₀ for one or more WT cells, e.g., WT cells of the same species and tissue type as the JAK-mediated disease cells.

In some aspects, provided herein is a method for identifying a heterobifunctional compound which mediates degradation or reduction of JAK, the method comprising: providing a heterobifunctional test compound comprising an JAK ligand conjugated to a degradation tag through a linker; contacting the heterobifunctional test compound with a cell comprising a ubiquitin ligase and JAK; determining whether JAK level is decreased in the cell; and identifying the heterobifunctional test compound as a heterobifunctional compound which mediates degradation or reduction of JAK. In certain embodiments, the cell is a cancer cell. In certain embodiments, the cancer cell is a JAK-mediated cancer cell.

Synthesis and Testing of Heterobifunctional Compounds

The binding affinity of novel synthesized heterobifunctional compounds can be assessed using standard biophysical assays known in the art (e.g., isothermal titration calorimetry (ITC), surface plasmon resonance (SPR)). Cellular assays can then be used to assess the heterobifunctional compound's ability to induce JAK (e.g. JAK1, JAK2, JAK3, and TYK2) degradation and inhibit cancer cell proliferation. Besides evaluating a heterobifunctional compound's induced changes in the protein levels of JAK, JAK mutants, JAK deletions, or JAK fusion proteins, protein-protein interaction or acteryltransferase enzymatic activity can also be assessed. Assays suitable for use in any or all of these steps are known in the art, and include, e.g., western blotting, quantitative mass spectrometry (MS) analysis, flow cytometry, enzymatic activity assay, ITC, SPR, cell growth inhibition, xenograft, orthotopic, and patient-derived xenograft models. Suitable cell lines for use in any or all of these steps are known in the art and include HEL, RS4;11, MV4;11, MOLT-4, CCRF-CEM, Kasumi-1, MM.1S, HL-60, WSU-DLCL2, Pfeiffer, and SU-DHL-1 cancer cell lines. Suitable mouse models for use in any or all of these steps are known in the art and include subcutaneous xenograft models, orthotopic models, patient-derived xenograft models, and patient-derived orthotopic models.

By way of non-limiting example, detailed synthesis protocols are described in the Examples for specific exemplary heterobifunctional compounds.

Pharmaceutically acceptable isotopic variations of the compounds disclosed herein are contemplated and can be synthesized using conventional methods known in the art or methods corresponding to those described in the Examples (substituting appropriate reagents with appropriate isotopic variations of those reagents). Specifically, an isotopic variation is a compound in which at least one atom is replaced by an atom having the same atomic number, but an atomic mass different from the atomic mass usually found in nature. Useful isotopes are known in the art and include, for example, isotopes of hydrogen, carbon, nitrogen, oxygen, phosphorus, sulfur, fluorine, and chlorine. Exemplary isotopes thus include, e.g., ²H, ³H, ¹³C, ¹⁴C, ¹⁵N, ¹⁷O, ¹⁸O, ³²P, ³⁵S, ¹⁸F, and ³⁶Cl.

Isotopic variations (e.g., isotopic variations containing ²H) can provide therapeutic advantages resulting from greater metabolic stability, e.g., increased in vivo half-life or reduced dosage requirements. In addition, certain isotopic variations (particularly those containing a radioactive isotope) can be used in drug or substrate tissue distribution studies. The radioactive isotopes tritium (³H) and carbon-14 (¹⁴C) are particularly useful for this purpose in view of their ease of incorporation and ready means of detection.

Pharmaceutically acceptable solvates of the compounds disclosed herein are contemplated. A solvate can be generated, e.g., by substituting a solvent used to crystallize a compound disclosed herein with an isotopic variation (e.g., D₂O in place of H₂O, d₆-acetone in place of acetone, or d₆-DMSO in place of DMSO).

Pharmaceutically acceptable fluorinated variations of the compounds disclosed herein are contemplated and can be synthesized using conventional methods known in the art or methods corresponding to those described in the Examples (substituting appropriate reagents with appropriate fluorinated variations of those reagents). Specifically, a fluorinated variation is a compound in which at least one hydrogen atom is replaced by a fluoro atom. Fluorinated variations can provide therapeutic advantages resulting from greater metabolic stability, e.g., increased in vivo half-life or reduced dosage requirements.

Pharmaceutically acceptable prodrugs of the compounds disclosed herein are contemplated and can be synthesized using conventional methods known in the art or methods corresponding to those described in the Examples (e.g., converting hydroxyl groups or carboxylic acid groups to ester groups). As used herein, a “prodrug” refers to a compound that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

Characterization of Exemplary Heterobifunctional Compounds

Specific exemplary heterobifunctional compounds were first characterized using cell viability assays. RS4;11 ALL cells were treated with bifunctional degraders for three days. The IC₅₀ values ranged from 2 nM to over 10 uM. Compounds JA-093, JA-094, JA-179, JA-180, JA-182, JA-187, JA-188, JA-189, JA-196, JA-198, JA-199, JA-202, JA-203, JA-213, JA-214, JA-224, JA-225, JA-231 showed significant cell viability inhibition activity with IC₅₀ values below 100 nM (Table 1). JA-189 and JA-213 were further characterized in a variety of additional cell lines shown in Table 2. Compared to JAK inhibitors, NVP-BSK805 and TG101209, JA-189 and JA-213 showed significant cell viability inhibition activity in RS4;11, HEL, MOLT-4, CCRF-CEM, MV4;11, Kasumi-1, MM.1S, HL-60, WSU-DLCL2, Pfeiffer and SU-DHL-1 cell lines (Table 2).

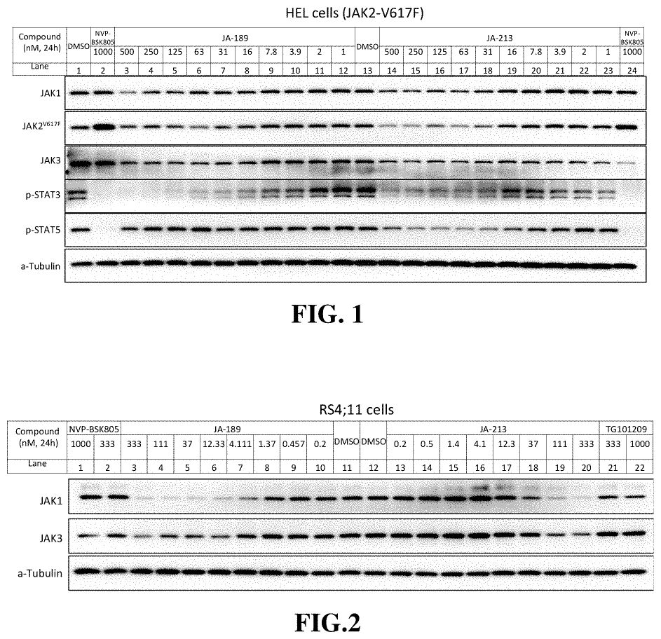

To confirm JA-189 and JA-213 are true JAK degraders, HEL cell were treated with these two compounds at various compounds concentrations. Comparing with NVP-BSK805 and DMSO control, JA-189 and JA-213 were able to reduce JAK1, JAK2 and JAK3 protein levels, as well as down stream signaling as demonstrated by the weaker bands of p-STAT3 and p-STAT5 (FIG. 1). In RS4;11 cells, JA-189 and JA-213 showed even better activity in reducing the JAK1 and JAK3 protein levels (FIG. 2).

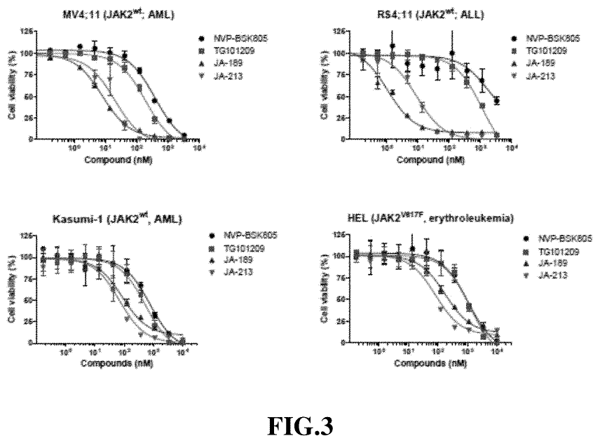

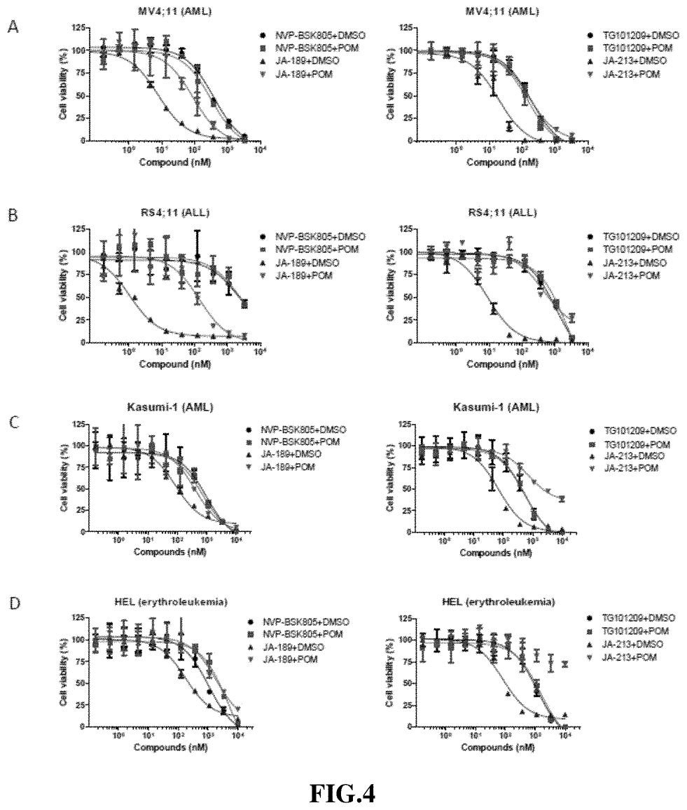

Comparing with NVP-BSK805 and G101209, JA-189 and JA-213 showed significant improvement at inhibiting the viability of MV4;11, MS4;11, Kasumi-1, and HEL cell lines (FIG. 3). We further demonstrated that pomalidomide (POM) and degrader (JA-189 or JA-213) combination treatment greatly reduce the potencies of the degraders in MV4;11, MS4;11, Kasumi-1, and HEL cells (FIG. 4). Since pomalidomide competes with the degraders at the CRBN binding site, pomalidomide combination will reduce the CRBN binding of the degraders, thus diminishing the JAK protein degradation. This experiment demonstrated that CRBN binding is required for the observed cell viability inhibition activities of degraders, JA-189 and JA-213.

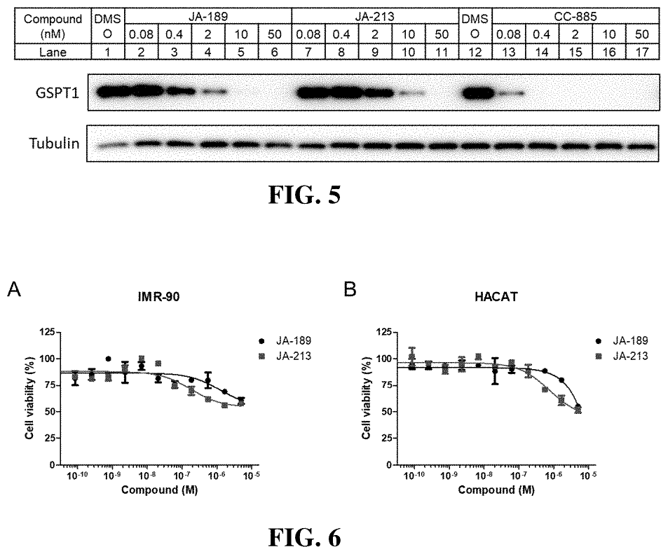

Our results further showed that some of the heterobifunctional molecules, as exemplified by JA-189 and JA-213, also induced degradation of GSPT1 in a dose dependent manner (FIG. 5).

In addition, immortalized human lung fibroblast IMR-90 and keratinocyte HACAT cells were not sensitive to JA-189 and JA-213 (FIG. 6). These non-cancerous human cell growth inhibition data indicate potential therapeutic windows of these degraders.

### Definition of Terms

As used herein, the terms “comprising” and “including” are used in their open, non-limiting sense.

“Alkyl” refers to a straight or branched hydrocarbon chain radical consisting solely of carbon and hydrogen atoms, containing no unsaturation. An alkyl may comprise one, two, three, four, five, six, seven, eight, nine, ten, eleven, twelve, thirteen, fourteen, fifteen, or sixteen carbon atoms. In certain embodiments, an alkyl comprises one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), pentyl, 3-methylhexyl, 2-methylhexyl, and the like.

“Alkenyl” refers to a straight or branched hydrocarbon chain radical group consisting solely of carbon and hydrogen atoms, containing at least one double bond. An alkenyl may comprise two, three, four, five, six, seven, eight, nine, ten, eleven, twelve, thirteen, fourteen, fifteen, or sixteen carbon atoms. In certain embodiments, an alkenyl comprises two to twelve carbon atoms (e.g., C₂-C₁₂ alkenyl). In certain embodiments, an alkenyl comprises two to eight carbon atoms (e.g., C₂-C₈ alkenyl). In certain embodiments, an alkenyl comprises two to six carbon atoms (e.g., C₂-C₆ alkenyl). In other embodiments, an alkenyl comprises two to four carbon atoms (e.g., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

The term “allyl,” as used herein, means a —CH₂CH═CH₂ group.

As used herein, the term “alkynyl” refers to a straight or branched hydrocarbon chain radical group consisting solely of carbon and hydrogen atoms, containing at least one triple bond. An alkynyl may comprise two, three, four, five, six, seven, eight, nine, ten, eleven, twelve, thirteen, fourteen, fifteen, or sixteen carbon atoms. In certain embodiments, an alkynyl comprises two to twelve carbon atoms (e.g., C₂-C₁₂ alkynyl). In certain embodiments, an alkynyl comprises two to eight carbon atoms (e.g., C₂-C₈ alkynyl). In other embodiments, an alkynyl has two to six carbon atoms (e.g., C₂-C₆ alkynyl). In other embodiments, an alkynyl has two to four carbon atoms (e.g., C₂-C₄ alkynyl). The alkynyl is attached to the rest of the molecule by a single bond. Examples of such groups include, but are not limited to, ethynyl, propynyl, 1-butynyl, 2-butynyl, 1-pentynyl, 2-pentynyl, 1-hexynyl, 2-hexynyl, 3-hexynyl, and the like.

The term “alkoxy”, as used herein, means an alkyl group as defined herein which is attached to the rest of the molecule via an oxygen atom. Examples of such groups include, but are not limited to, methoxy, ethoxy, n-propyloxy, iso-propyloxy, n-butoxy, iso-butoxy, tert-butoxy, pentyloxy, hexyloxy, and the like.

The term “aryl”, as used herein, “refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon atoms. An aryl may comprise from six to eighteen carbon atoms, where at least one of the rings in the ring system is fully unsaturated, i.e., it contains a cyclic, delocalized (4n+2) π-electron system in accordance with the Hückel theory. In certain embodiments, an aryl comprises six to fourteen carbon atoms (C₆-C₁₄ aryl or 6-14 membered aryl). In certain embodiments, an aryl comprises six to ten carbon atoms (C₆-C₁₀ aryl or 6-10 membered aryl). Examples of such groups include, but are not limited to, phenyl, fluorenyl and naphthyl. The terms “Ph” and “phenyl,” as used herein, mean a —C₆H₅ group.

The term “heteroaryl”, refers to a radical derived from a 3- to 18-membered aromatic ring radical (i.e. 3-18 membered heteroaryl) that comprises two to seventeen carbon atoms and from one to six heteroatoms selected from nitrogen, oxygen and sulfur. As used herein, the heteroaryl radical may be a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In certain embodiments, a heteroaryl refers to a radical derived from a 3- to 10-membered aromatic ring radical (3-10 membered heteroaryl). In certain embodiments, a heteroaryl refers to a radical derived from 5- to 7-membered aromatic ring (5-7 membered heteroaryl). Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of such groups include, but not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and the like. In certain embodiments, a heteroaryl is attached to the rest of the molecule via a ring carbon atom. In certain embodiments, an heteroaryl is attached to the rest of the molecule via a nitrogen atom (N-attached) or a carbon atom (C-attached). For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached).

The term “heterocyclyl”, as used herein, means a non-aromatic, monocyclic, bicyclic, tricyclic, or tetracyclic radical having a total of from 4, 5, 6, 7, 8, 9, 10, 11, 12, or 13 atoms in its ring system, and containing from 3 to 12 carbon atoms and from 1 to 4 heteroatoms each independently selected from O, S and N, and with the proviso that the ring of said group does not contain two adjacent O atoms or two adjacent S atoms. A heterocyclyl group may include fused, bridged or spirocyclic ring systems. In certain embodiments, a heterocyclyl group comprises 3 to 10 ring atoms (3-10 membered heterocyclyl). In certain embodiments, a heterocyclyl group comprises 3 to 8 ring atoms (3-8 membered heterocyclyl). In certain embodiments, a heterocyclyl group comprises 4 to 10 ring atoms (4-10 membered heterocyclyl). In certain embodiments, a heterocyclyl group comprises 4 to 8 ring atoms (4-8 membered heterocyclyl). A heterocyclyl group may contain an oxo substituent at any available atom that will result in a stable compound. For example, such a group may contain an oxo atom at an available carbon or nitrogen atom. Such a group may contain more than one oxo substituent if chemically feasible. In addition, it is to be understood that when such a heterocyclyl group contains a sulfur atom, said sulfur atom may be oxidized with one or two oxygen atoms to afford either a sulfoxide or sulfone. An example of a 4 membered heterocyclyl group is azetidinyl (derived from azetidine). An example of a 5 membered cycloheteroalkyl group is pyrrolidinyl. An example of a 6 membered cycloheteroalkyl group is piperidinyl. An example of a 9 membered cycloheteroalkyl group is indolinyl. An example of a 10 membered cycloheteroalkyl group is 4H-quinolizinyl. Further examples of such heterocyclyl groups include, but are not limited to, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, 3-oxopiperazinyl, 4-methylpiperazinyl, 4-ethylpiperazinyl, and 1-oxo-2,8, diazaspiro[4.5]dec-8-yl. A heteroaryl group may be attached to the rest of molecular via a carbon atom (C-attached) or a nitrogen atom (N-attached). For instance, a group derived from piperazine may be piperazin-1-yl (N-attached) or piperazin-2-yl (C-attached).

The term “cycloalkyl” or “carbocyclyl” means a saturated, monocyclic, bicyclic, tricyclic, or tetracyclic radical having a total of from 4, 5, 6, 7, 8, 9, 10, 11, 12, or 13 carbon atoms in its ring system. A cycloalkyl may be fused, bridged or spirocyclic. In certain embodiments, a cycloalkyl comprises 3 to 8 carbon ring atoms (3-8 membered carbocyclyl). In certain embodiments, a cycloalkyl comprises 3 to 10 carbon ring atoms (3-10 membered cycloalkyl). Examples of such groups include, but are not limited to, cyclopropyl, cyclobutyl, cyclopentyl, cyclopentenyl, cyclohexyl, cycloheptyl, adamantyl, and the like.

The term “cycloalkylene” is a bidentate radical obtained by removing a hydrogen atom from a cycloalkyl ring as defined above. Examples of such groups include, but are not limited to, cyclopropylene, cyclobutylene, cyclopentylene, cyclopentenylene, cyclohexylene, cycloheptylene, and the like.

The term “spirocyclic” as used herein has its conventional meaning, that is, any ring system containing two or more rings wherein two of the rings have one ring carbon in common. Each ring of the spirocyclic ring system, as herein defined, independently comprises 3 to 20 ring atoms. Preferably, they have 3 to 10 ring atoms. Non-limiting examples of a spirocyclic system include spiro[3.3]heptane, spiro[3.4]octane, and spiro[4.5]decane.

The term cyano” refers to a —C≡N group.

An “aldehyde” group refers to a —C(O)H group.

An “alkoxy” group refers to both an —O-alkyl, as defined herein.

An “alkoxycarbonyl” refers to a —C(O)-alkoxy, as defined herein.

An “alkylaminoalkyl” group refers to an -alkyl-NR-alkyl group, as defined herein.

An “alkylsulfonyl” group refer to a —SO₂alkyl, as defined herein.

An “amino” group refers to an optionally substituted —NH₂.

An “aminoalkyl” group refers to an -alky-amino group, as defined herein.

An “aminocarbonyl” refers to a —C(O)-amino, as defined herein.

An “arylalkyl” group refers to -alkylaryl, where alkyl and aryl are defined herein.

An “aryloxy” group refers to both an —O-aryl and an —O-heteroaryl group, as defined herein.

An “aryloxycarbonyl” refers to —C(O)-aryloxy, as defined herein.

An “arylsulfonyl” group refers to a —SO₂aryl, as defined herein.

A “carbonyl” group refers to a —C(O)— group, as defined herein.

A “carboxylic acid” group refers to a —C(O)OH group.

A “cycloalkoxy” refers to a —O-cycloalkyl group, as defined herein.

A “halo” or “halogen” group refers to fluorine, chlorine, bromine or iodine.

A “haloalkyl” group refers to an alkyl group substituted with one or more halogen atoms.

A “hydroxy” group refers to an —OH group.

A “nitro” group refers to a —NO₂ group.

An “oxo” group refers to the ═O substituent.

A “trihalomethyl” group refers to a methyl substituted with three halogen atoms.

The term “length” when refers to a moiety means the smallest number of carbon and/or hetero atoms from one end to the other end of the moiety. When it refers to the linker, it means the smallest number of atoms from the end connects to the TRK ligand and the end connects to the degradation tag. It applies to both situations where the linker is linear or branched, and where the linker comprises a ring system.

The term “substituted,” means that the specified group or moiety bears one or more substituents independently selected from C₁-C₄ alkyl, aryl, heteroaryl, aryl-C₁-C₄ alkyl-, heteroaryl-C₁-C₄ alkyl-, C₁-C₄ haloalkyl, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo, —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ alkylphenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “null” means the absence of an atom or moiety, and there is a bond between adjacent atoms in the structure.

The term “optionally substituted” means that the specified group may be either unsubstituted or substituted by one or more substituents as defined herein. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆ aryl group, also called “phenyl” herein, is substituted with one additional substituent, one of ordinary skill in the art would understand that such a group has 4 open positions left on carbon atoms of the C₆ aryl ring (6 initial positions, minus one at which the remainder of the compound of the present invention is attached to and an additional substituent, remaining 4 positions open). In such cases, the remaining 4 carbon atoms are each bound to one hydrogen atom to fill their valencies. Similarly, if a C₆ aryl group in the present compounds is said to be “disubstituted,” one of ordinary skill in the art would understand it to mean that the C₆ aryl has 3 carbon atoms remaining that are unsubstituted. Those three unsubstituted carbon atoms are each bound to one hydrogen atom to fill their valencies. Unless otherwise specified, an optionally substituted radical may be a radical unsubstituted or substituted with one or more substituents selected from halogen, CN, NO₂, OR^{m}, SR^{m}, NRⁿR^{o}, COR^{m}, CO₂R^{m}, CONRⁿR^{o}, SOR^{m}, SO₂R^{m}, SO₂NRⁿR^{o}, NRⁿCOR^{o}, NR^{m}C(O)NRⁿR^{o}, NRⁿSOR^{o}, NRⁿSO₂R^{o}, C₁-C₈ alkyl, C₁-C₈alkoxyC₁-C₈alkyl, C₁-C₈ haloalkyl, C₁-C₈ hydroxyalkyl, C₁-C₈alkylaminoC₁-C₈ alkyl, C₃-C₇ cycloalkyl, 3-7 membered heterocyclyl, C₂-C₈ alkenyl, C₂-C₈ alkynyl, aryl, and heteroaryl, wherein R^{m}, Rⁿ, and R^{o} are independently selected from null, hydrogen, C₁-C₈ alkyl, C₂-C₈ alkenyl, C₂-C₈ alkynyl, C₃-C₇ cycloalkyl, 3-7 membered heterocyclyl, aryl, and heteroaryl, or Rⁿ and R^{o} together with the atom to which they are connected form a 3-8 membered cycloalkyl or heterocyclyl ring.

As used herein, the same symbol in different FORMULA means different definition, for example, the definition of R1 in FORMULA 1 is as defined with respect to FORMULA 1 and the definition of R1 in FORMULA 6 is as defined with respect to FORMULA 6.

As used herein, each unit in the linker moiety

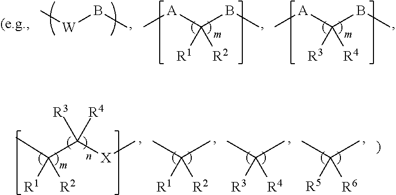

can be the same as or different from each other. In certain embodiments, each unit in the linker moiety is the same as each other.

As used herein, when m (or n or o or p) is definited by a range, for example, “m is 0 to 15” or “m=0-3” mean that m is an integer from 0 to 15 (i.e. m is 0, 1, 2, 3, 4, 5, 6, 7, 8, 9, 10, 11, 12, 13, 14, or 15) or m is an integer from 0 to 3 (i.e. m is 0, 1, 2, or 3) or is any integer in the defined range.

“Pharmaceutically acceptable salt” includes both acid and base addition salts. A pharmaceutically acceptable salt of any one of the heterobifunctional compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997), which is hereby incorporated by reference in its entirety). Acid addition salts of basic compounds may be prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

Pharmaceutical Compositions

In some aspects, the compositions and methods described herein include the manufacture and use of pharmaceutical compositions and medicaments that include one or more heterobifunctional compounds as disclosed herein. Also included are the pharmaceutical compositions themselves.

In some aspects, the compositions disclosed herein can include other compounds, drugs, or agents used for the treatment of cancer. For example, in some instances, pharmaceutical compositions disclosed herein can be combined with one or more (e.g., one, two, three, four, five, or less than ten) compounds. Such additional compounds can include, e.g., conventional chemotherapeutic agents or any other cancer treatment known in the art. When co-administered, heterobifunctional compounds disclosed herein can operate in conjunction with conventional chemotherapeutic agents or any other cancer treatment known in the art to produce mechanistically additive or synergistic therapeutic effects.

In some aspects, the pH of the compositions disclosed herein can be adjusted with pharmaceutically acceptable acids, bases, or buffers to enhance the stability of the heterobifunctional compound or its delivery form.

Pharmaceutical compositions typically include a pharmaceutically acceptable excipient, adjuvant, or vehicle. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human. A pharmaceutically acceptable excipient, adjuvant, or vehicle is a substance that can be administered to a patient, together with a compound of the invention, and which does not compromise the pharmacological activity thereof and is nontoxic when administered in doses sufficient to deliver a therapeutic amount of the compound. Exemplary conventional nontoxic pharmaceutically acceptable excipients, adjuvants, and vehicles include, but not limited to, saline, solvents, dispersion media, coatings, antibacterial and antifungal agents, isotonic and absorption delaying agents, and the like, compatible with pharmaceutical administration.

In particular, pharmaceutically acceptable excipients, adjuvants, and vehicles that can be used in the pharmaceutical compositions of this invention include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

Depending on the dosage form selected to deliver the heterobifunctional compounds disclosed herein, different pharmaceutically acceptable excipients, adjuvants, and vehicles may be used. In the case of tablets for oral use, pharmaceutically acceptable excipients, adjuvants, and vehicles may be used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening, flavoring, or coloring agents can be added.

As used herein, the heterobifunctional compounds disclosed herein are defined to include pharmaceutically acceptable derivatives or prodrugs thereof. A “pharmaceutically acceptable derivative” means any pharmaceutically acceptable salt, solvate, or prodrug, e.g., carbamate, ester, phosphate ester, salt of an ester, or other derivative of a compound or agent disclosed herein, which upon administration to a recipient is capable of providing (directly or indirectly) a compound described herein, or an active metabolite or residue thereof. Particularly favored derivatives and prodrugs are those that increase the bioavailability of the compounds disclosed herein when such compounds are administered to a subject (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group that enhances aqueous solubility or active transport through the gut membrane is appended to the structure of formulae described herein. Such derivatives are recognizable to those skilled in the art without undue experimentation. Nevertheless, reference is made to the teaching of Burger's Medicinal Chemistry and Drug Discovery, 5^{th} Edition, Vol. 1: Principles and Practice, which is incorporated herein by reference to the extent of teaching such derivatives.

The heterobifunctional compounds disclosed herein include pure enantiomers, mixtures of enantiomers, pure diastereoisomers, mixtures of diastereoisomers, diastereoisomeric racemates, mixtures of diastereoisomeric racemates and the meso-form and pharmaceutically acceptable salts, solvent complexes, morphological forms, or deuterated derivatives thereof.

In some aspects, the pharmaceutical compositions disclosed herein can include an effective amount of one or more heterobifunctional compounds. The terms “effective amount” and “effective to treat,” as used herein, refer to an amount or a concentration of one or more compounds or a pharmaceutical composition described herein utilized for a period of time (including acute or chronic administration and periodic or continuous administration) that is effective within the context of its administration for causing an intended effect or physiological outcome (e.g., treatment or prevention of cell growth, cell proliferation, or cancer). In some aspects, pharmaceutical compositions can further include one or more additional compounds, drugs, or agents used for the treatment of cancer (e.g., conventional chemotherapeutic agents) in amounts effective for causing an intended effect or physiological outcome (e.g., treatment or prevention of cell growth, cell proliferation, or cancer).

In some aspects, the pharmaceutical compositions disclosed herein can be formulated for sale in the United States, import into the United States, or export from the United States.

Administration of Pharmaceutical Compositions

The pharmaceutical compositions disclosed herein can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs). In particular, the pharmaceutical compositions can be formulated for and administered via oral, parenteral, or transdermal delivery. The term “parenteral” as used herein includes subcutaneous, intracutaneous, intravenous, intramuscular, intraperitoneal, intra-articular, intra-arterial, intrasynovial, intrasternal, intrathecal, intralesional, and intracranial injection or infusion techniques.

For example, the pharmaceutical compositions disclosed herein can be administered, e.g., topically, rectally, nasally (e.g., by inhalation spray or nebulizer), buccally, vaginally, subdermally (e.g., by injection or via an implanted reservoir), or ophthalmically.

For example, pharmaceutical compositions of this invention can be orally administered in any orally acceptable dosage form including, but not limited to, capsules, tablets, emulsions and aqueous suspensions, dispersions and solutions.

For example, the pharmaceutical compositions of this invention can be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax, and polyethylene glycols.

For example, the pharmaceutical compositions of this invention can be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, or other solubilizing or dispersing agents known in the art.

For example, the pharmaceutical compositions of this invention can be administered by injection (e.g., as a solution or powder). Such compositions can be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, e.g., as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil can be employed, including synthetic mono- or diglycerides. Fatty acids, such as oleic acid and its glyceride derivatives are useful in the preparation of injectables, as are natural pharmaceutically-acceptable oils, e.g., olive oil or castor oil, especially in their polyoxyethylated versions. These oil solutions or suspensions can also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens, Spans, or other similar emulsifying agents or bioavailability enhancers which are commonly used in the manufacture of pharmaceutically acceptable solid, liquid, or other dosage forms can also be used for the purposes of formulation.

In some aspects, an effective dose of a pharmaceutical composition of this invention can include, but is not limited to, e.g., about 0.00001, 0.0001, 0.001, 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.5, 3, 4, 5, 6, 7, 8, 9, 10, 15, 20, 30, 40, 50, 60, 70, 80, 90, 100, 200, 300, 400, 500, 600, 700, 800, 900, 1000, 2500, 5000, or 10000 mg/kg/day, or according to the requirements of the particular pharmaceutical composition.

When the pharmaceutical compositions disclosed herein include a combination of the heterobifunctional compounds described herein and one or more additional compounds (e.g., one or more additional compounds, drugs, or agents used for the treatment of cancer or any other condition or disease, including conditions or diseases known to be associated with or caused by cancer), both the heterobifunctional compounds and the additional compounds may be present at dosage levels of between about 1 to 100%, and more preferably between about 5 to 95% of the dosage normally administered in a monotherapy regimen. The additional agents can be administered separately, as part of a multiple dose regimen, from the compounds of this invention. Alternatively, those agents can be part of a single dosage form, mixed together with the compounds of this invention in a single composition.

In some aspects, the pharmaceutical compositions disclosed herein can be included in a container, pack, or dispenser together with instructions for administration.

Methods of Treatment

The methods disclosed herein contemplate administration of an effective amount of a compound or composition to achieve the desired or stated effect. Typically, the compounds or compositions of the invention will be administered from about 1 to about 6 times per day or, alternately or in addition, as a continuous infusion. Such administration can be used as a chronic or acute therapy. The amount of active ingredient that can be combined with the carrier materials to produce a single dosage form will vary depending upon the host treated and the particular mode of administration. A typical preparation will contain from about 5% to about 95% active compound (w/w). Alternatively, such preparations can contain from about 20% to about 80% active compound.

In some aspects, provided herein are a heterobifunctional compound described herein for preventing or treating a disease or condition.

In some aspects, provided herein are a heterobifunctional compound described herein for treating or preventing one or more diseases or conditions disclosed herein in a subject in need thereof. In certain embodiments, the disease or condition is a JAK-mediated disease or condition. In certain embodiments, the disease or condition is resulted from JAK expression, mutation, deletion, or fusion. In certain embodiments, the diseases or conditions are cancer, inflammation, auto-immune disease, viral infections, and immunological diseases. In one embodiment, the JAK-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, and myeloma. In one embodiment, the JAK-mediated inflammatory disorders are selected from the group consisting of ankylosing spondylitis, Crohn's disease, inflammatory bowel disease, ulcerative colitis, and ischemia reperfusion injuries. In one embodiment, the JAK-mediated auto-immune diseases are selected from the group consisting of multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid. In one embodiment, the JAK-mediated dermatological disorders are selected from the group consisting of atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING-associated vasculopathy. In one embodiment, the JAK-mediated viral infections are selected from the group consisting of infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV). In one embodiment, the JAK-mediated dry eye disorders are selected from the group consisting of dry eye syndrome (DES) and keratoconjunctivitis sicca (KCS). In one embodiment, the JAK-mediated bone remodeling disorders are selected from the group consisting of osteoporosis and osteoarthritis. In one embodiment, the JAK-mediated organ transplant associated immunological complications are selected from the group consisting of graft-versus-host diseases.

In some aspects, provided herein are use of a heterobifunctional compound in manufacture of a medicament for preventing or treating one or more diseases or conditions disclosed herein.

In some aspects, the methods disclosed include the administration of a therapeutically effective amount of one or more of the compounds or compositions described herein to a subject (e.g., a mammalian subject, e.g., a human subject) who is in need of, or who has been determined to be in need of, such treatment. In some aspects, the methods disclosed include selecting a subject and administering to the subject an effective amount of one or more of the compounds or compositions described herein, and optionally repeating administration as required for the prevention or treatment of cancer.

In some aspects, subject selection can include obtaining a sample from a subject (e.g., a candidate subject) and testing the sample for an indication that the subject is suitable for selection. In some aspects, the subject can be confirmed or identified, e.g. by a health care professional, as having had, having an elevated risk to have, or having a condition or disease. In some aspects, suitable subjects include, for example, subjects who have or had a condition or disease but that resolved the disease or an aspect thereof, present reduced symptoms of disease (e.g., relative to other subjects (e.g., the majority of subjects) with the same condition or disease), or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some aspects, exhibition of a positive immune response towards a condition or disease can be made from patient records, family history, or detecting an indication of a positive immune response. In some aspects, multiple parties can be included in subject selection. For example, a first party can obtain a sample from a candidate subject and a second party can test the sample. In some aspects, subjects can be selected or referred by a medical practitioner (e.g., a general practitioner). In some aspects, subject selection can include obtaining a sample from a selected subject and storing the sample or using the in the methods disclosed herein. Samples can include, e.g., cells or populations of cells.

In some aspects, methods of treatment can include a single administration, multiple administrations, and repeating administration of one or more compounds disclosed herein as required for the prevention or treatment of the disease or condition disclosed herein (e.g., an JAK-mediated disease). In some aspects, methods of treatment can include assessing a level of disease in the subject prior to treatment, during treatment, or after treatment. In some aspects, treatment can continue until a decrease in the level of disease in the subject is detected.

The term “subject,” as used herein, refers to any animal. In some instances, the subject is a mammal. In some instances, the term “subject,” as used herein, refers to a human (e.g., a man, a woman, or a child).

The terms “administer,” “administering,” or “administration,” as used herein, refer to implanting, ingesting, injecting, inhaling, or otherwise absorbing a compound or composition, regardless of form. For example, the methods disclosed herein include administration of an effective amount of a compound or composition to achieve the desired or stated effect.

The terms “treat”, “treating,” or “treatment,” as used herein, refer to partially or completely alleviating, inhibiting, ameliorating, or relieving the disease or condition from which the subject is suffering. This means any manner in which one or more of the symptoms of a disease or disorder (e.g., cancer) are ameliorated or otherwise beneficially altered. As used herein, amelioration of the symptoms of a particular disorder (e.g., cancer) refers to any lessening, whether permanent or temporary, lasting or transient that can be attributed to or associated with treatment by the heterobifunctional compounds, compositions and methods of the present invention. In some embodiments, treatment can promote or result in, for example, a decrease in the number of tumor cells (e.g., in a subject) relative to the number of tumor cells prior to treatment; a decrease in the viability (e.g., the average/mean viability) of tumor cells (e.g., in a subject) relative to the viability of tumor cells prior to treatment; a decrease in the rate of growth of tumor cells; a decrease in the rate of local or distant tumor metastasis; or reductions in one or more symptoms associated with one or more tumors in a subject relative to the subject's symptoms prior to treatment.

The terms “prevent,” “preventing,” and “prevention,” as used herein, shall refer to a decrease in the occurrence of a disease or decrease in the risk of acquiring a disease or its associated symptoms in a subject. The prevention may be complete, e.g., the total absence of disease or pathological cells in a subject. The prevention may also be partial, such that the occurrence of the disease or pathological cells in a subject is less than, occurs later than, or develops more slowly than that which would have occurred without the present invention. In certain embodiments, the subject has an elevated risk of developing one or more JAK-mediated diseases. Exemplary JAK-mediated diseases that can be treated with heterobifunctional compounds include, for example, cancer (e.g. cancers of brain, stomach, gastrointestinal tracts, liver, biliary passage, breast, ovary, cervix, prostate, testis, penile, genitourinary tract, esophagus, larynx, skin, lung, pancreas, thyroid, glands, bladder, kidney, muscle, bone, and cancers of the hematopoietic system, such as myeloproliferative neoplasms, including essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, myeloma, and other hematologic malignancies, particularly cancers that involve inflammation, mutations or other aberrations that activate the JAK pathway); inflammation (e.g. ankylosing spondylitis, Crohn's disease, inflammatory bowel disease, ulcerative colitis, and ischemia reperfusion injuries, which are conditions related to inflammatory ischemic events such as stroke or cardiac arrest); auto-immune diseases (e.g. multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid); dermatological disorders (e.g. atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING-associated vasculopathy); viral infections (e.g. viral infections and consequent complications, such as infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV)); dry eye disorder, also known as dry eye syndrome (DES) or keratoconjunctivitis sicca (KCS); bone remodeling disorders (e.g. osteoporosis and osteoarthritis); organ transplant associated immunological complications (e.g. graft-versus-host diseases).

Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

An effective amount can be administered in one or more administrations, applications or dosages. A therapeutically effective amount of a therapeutic compound (i.e., an effective dosage) depends on the therapeutic compounds selected. Moreover, treatment of a subject with a therapeutically effective amount of the compounds or compositions described herein can include a single treatment or a series of treatments. For example, effective amounts can be administered at least once. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health or age of the subject, and other diseases present.

Following administration, the subject can be evaluated to detect, assess, or determine their level of disease. In some instances, treatment can continue until a change (e.g., reduction) in the level of disease in the subject is detected. Upon improvement of a patient's condition (e.g., a change (e.g., decrease) in the level of disease in the subject), a maintenance dose of a compound, or composition disclosed herein can be administered, if necessary. Subsequently, the dosage or frequency of administration, or both, can be reduced, e.g., as a function of the symptoms, to a level at which the improved condition is retained. Patients may, however, require intermittent treatment on a long-term basis upon any recurrence of disease symptoms.

The present disclosure is also described and demonstrated by way of the following examples. However, the use of these and other examples anywhere in the specification is illustrative only and in no way limits the scope and meaning of the invention or of any exemplified term. Likewise, the invention is not limited to any particular preferred embodiment or aspect described herein. Indeed, many modifications and variations may be apparent to those skilled in the art upon reading this specification, and such variations can be made without departing from the invention in spirit or in scope. The invention is therefore to be limited only by the terms of the appended claims along with the full scope of equivalents to which those claims are entitled.

### EXAMPLES

### Example 1: 4-((2-Aminoethyl) amino)-2-(2,6-dioxopiperidin-3-yl) isoindoline-1,3-dione (Linker 1)

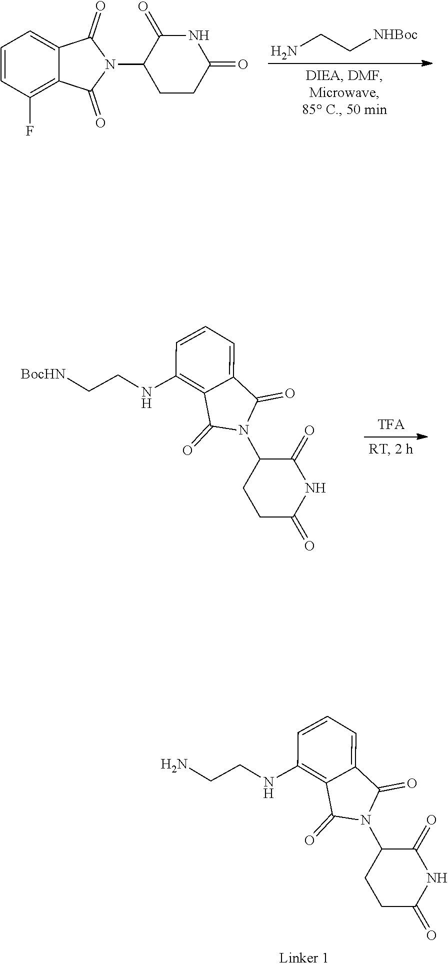 [SMILES: O=C1CCC(N2C(=O)c3cccc(F)c3C2=O)C(=O)N1 ; CC(C)(C)OC(=O)NCCN ; CC(C)(C)OC(=O)NCCNc1cccc2c1C(=O)N(C1CCC(=O)NC1=O)C2=O ; NCCNc1cccc2c1C(=O)N(C1CCC(=O)NC1=O)C2=O]

A solution of 2-(2,6-dioxopiperidin-3-yl)-4-fluoroisoindoline-1,3-dione (1.66 g, 6.0 mmol), tert-butyl (2-aminoethyl) carbamate (1.25 g, 6.6 mmol) and N,N-diisopropylethylamine (2.32 g, 18 mmmol) in DMF (12 mL) was heated to 85° C. in a microwave reactor for 50 min. Three batches were combined and diluted with EtOAc (200 mL). The reaction was washed with water and brine. The organic layer was dried over anhydrous sodium sulfate and concentrated under reduced pressure. The resulting residue was purified by silica gel chromatography (hexanes:EtOAc=1:1) to give tert-butyl (2-((2-(2,6-dioxopiperidin-3-yl)-1,3-dioxoisoindolin-4-yl)amino)ethyl)carbamate (1.3 g, yield: 16%) as yellow solid. MS (ESI) m/z=317.1 [M−100+H]⁺. A solution of tert-butyl (2-((2-(2,6-dioxopiperidin-3-yl)-1,3-dioxoisoindolin-4-yl) amino)ethyl)carbamate (2.0 g, 4.5 mmol) in DCM (10 mL) and TFA (5 mL) was stirred at rt for 2 h. The reaction was concentrated and triturated with EtOAc. The solid precipitate was filtered, washed with MTBE, and dried to give 4-((2-aminoethyl)amino)-2-(2,6-dioxopiperidin-3-yl)isoindoline-1,3-dione as yellow solid (Linker 1) (1.3 g, yield: 98%). ¹H NMR (400 MHz, DMSO-d₆) δ 11.14 (s, 1H), 7.85 (s, 3H), 7.45 (t, J=7.2 Hz, 1H), 7.19 (d, J=7.2 Hz, 1H), 7.10 (d, J=7.2 Hz, 1H), 6.84 (t, J=6.4 Hz, 1H), 5.07 (dd, J=5.2, 12.8 Hz, 1H), 3.58 (q, J=6.4 Hz, 2H), 3.00 (s, 2H), 2.94-2.85 (m, 1H), 2.62-2.50 (m, 2H), 2.05-2.00 (m, 1H). MS (ESI) m/z=317.1 [M+H]⁺.

### Example 2: 4-((3-Aminopropyl)amino)-2-(2,6-dioxopiperidin-3-yl)isoindoline-1,3-dione (Linker 2)

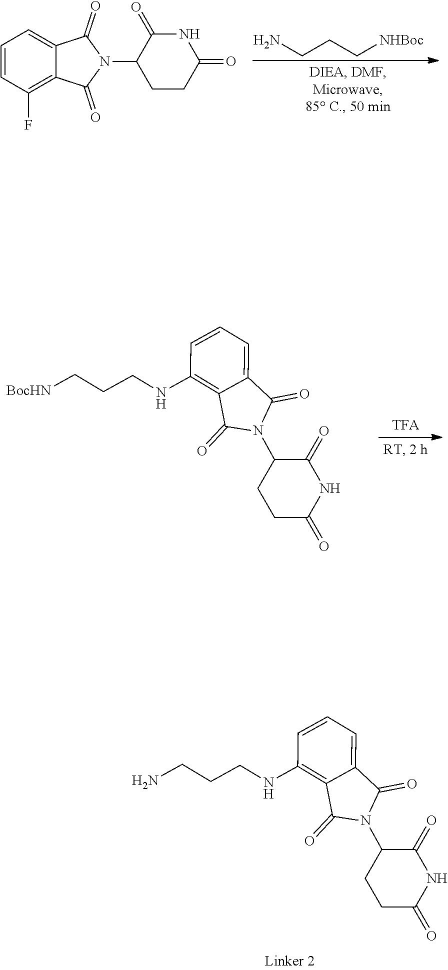 [SMILES: O=C1CCC(N2C(=O)c3cccc(F)c3C2=O)C(=O)N1 ; CC(C)(C)OC(=O)NCCCN ; CC(C)(C)OC(=O)NCCCNc1cccc2c1C(=O)N(C1CCC(=O)NC1=O)C2=O ; NCCCNc1cccc2c1C(=O)N(C1CCC(=O)NC1=O)C2=O]

Linker 2 was synthesized following the same procedures as Linker 1 as described for Example 1. (1.2 g, yield: 89%). ¹H NMR (400 MHz, DMSO-d₆) δ 11.11 (s, 1H), 7.74 (s, 3H), 7.62-7.58 (m, 1H), 7.15 (d, J=8.4 Hz, 1H), 7.05 (d, J=7.2 Hz, 1H), 6.78-6.75 (m, 1H), 5.08-5.04 (m, 1H), 3.43-3.36 (m, 2H), 2.90-2.86 (m, 3H), 2.62-2.51 (m, 2H), 2.08-2.01 (m, 1H), 1.86-1.80 (m, 2H). MS (ESI) m/z=331.1 [M+H]⁺.

### Example 3: 4-((4-Aminobutyl)amino)-2-(2,6-dioxopiperidin-3-yl)isoindoline-1,3-dione (Linker 3)

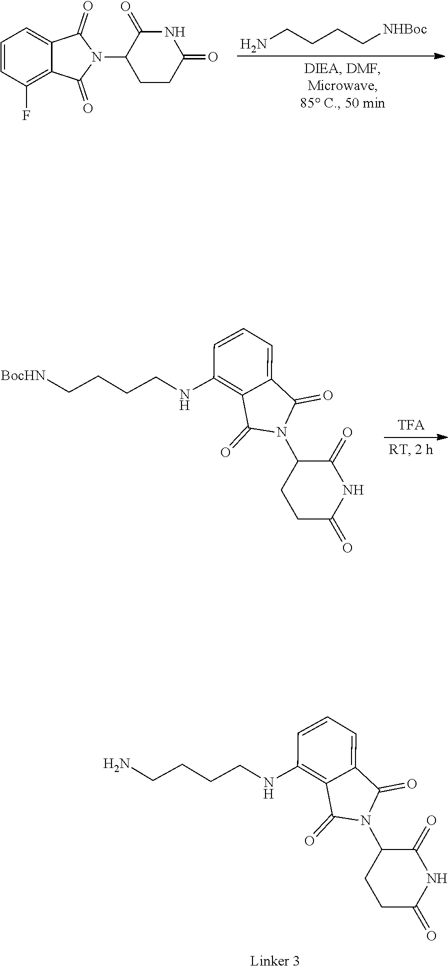 [SMILES: O=C1CCC(N2C(=O)c3cccc(F)c3C2=O)C(=O)N1 ; CC(C)(C)OC(=O)NCCCCN ; CC(C)(C)OC(=O)NCCCCNc1cccc2c1C(=O)N(C1CCC(=O)NC1=O)C2=O ; NCCCCNc1cccc2c1C(=O)N(C1CCC(=O)NC1=O)C2=O]

Linker 3 was synthesized following the same procedures as Linker 1 as described for Example 1. (1.4 g, yield: 93%). ¹H NMR (400 MHz, DMSO-d₆) δ 11.11 (s, 1H), 7.84 (s, 3H), 7.62-7.57 (m, 1H), 7.13 (d, J=8.4 Hz, 1H), 7.04 (d, J=6.8 Hz, 1H), 6.62 (s, 1H), 5.08-5.04 (m, 1H), 3.34 (s, 2H), 2.90-2.83 (m, 3H), 2.62-2.51 (m, 2H), 2.06-2.01 (m, 1H), 1.65-1.60 (m, 4H). MS (ESI) m/z=345.1 [M+H]⁺.

### Example 4: 4-((5-Aminopentyl)amino)-2-(2,6-dioxopiperidin-3-yl)isoindoline-1,3-dione (Linker 4)

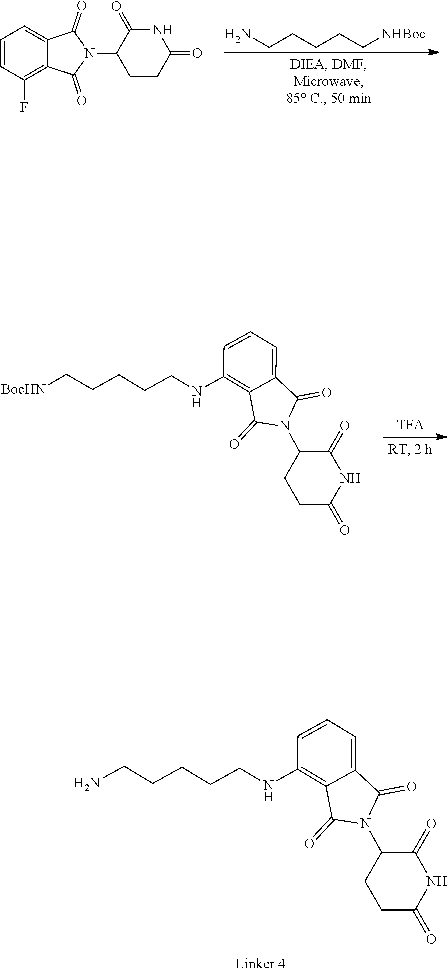 [SMILES: O=C1CCC(N2C(=O)c3cccc(F)c3C2=O)C(=O)N1 ; CC(C)(C)OC(=O)NCCCCCN ; CC(C)(C)OC(=O)NCCCCCNc1cccc2c1C(=O)N(C1CCC(=O)NC1=O)C2=O ; NCCCCCNc1cccc2c1C(=O)N(C1CCC(=O)NC1=O)C2=O]

Linker 4 was synthesized following the same procedures as Linker 1 as described for Example 1. (2.3 g, yield: 85%). ¹H NMR (400 MHz, DMSO-d₆) δ 11.14 (s, 1H), 7.72 (s, 3H), 7.61-7.57 (m, 1H), 7.10 (d, J=8.4 Hz, 1H), 7.03 (d, J=7.2 Hz, 1H), 6.56-6.53 (m, 1H), 5.07-5.03 (m, 1H), 3.32-3.28 (m, 2H), 2.90-2.78 (m, 3H), 2.62-2.51 (m, 2H), 2.05-1.90 (m, 1H), 1.62-1.54 (m, 4H), 1.41-1.37 (m, 2H). MS (ESI) m/z=359.1 [M+H]⁺.

### Example 5: 4-((6-Aminohexyl)amino)-2-(2,6-dioxopiperidin-3-yl)isoindoline-1,3-dione (Linker 5)

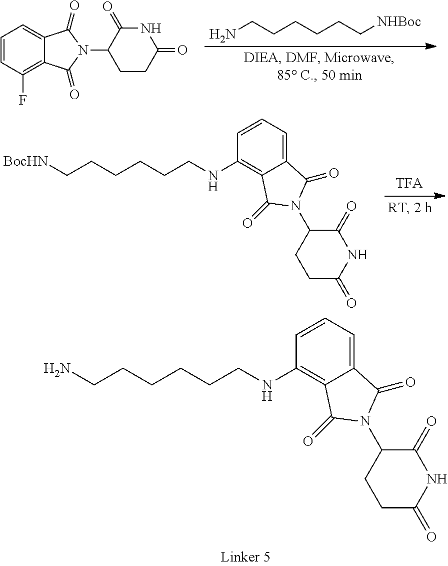

Linker 5 was synthesized following the same procedures as Linker 1 as described for Example 1. (1.8 g, yield: 67%). ¹H NMR (400 MHz, DMSO-d₆) δ 11.10 (s, 1H), 7.76 (s, 3H), 7.58 (t, J=7.2 Hz, 1H), 7.10 (d, J=8.4 Hz, 1H), 7.03 (d, J=7.2 Hz, 1H), 6.54 (t, J=6.0 Hz, 1H), 5.07-5.03 (m, 1H), 3.37-3.27 (m, 2H), 2.88-2.78 (m, 3H), 2.61-2.50 (m, 2H), 2.04-2.01 (m, 1H), 1.57-1.52 (m, 4H), 1.40-1.30 (m, 4H). MS (ESI) m/z=373.1 [M+H]⁺.

### Example 6: 4-((7-Aminoheptyl)amino)-2-(2,6-dioxopiperidin-3-yl)isoindoline-1,3-dione (Linker 6)

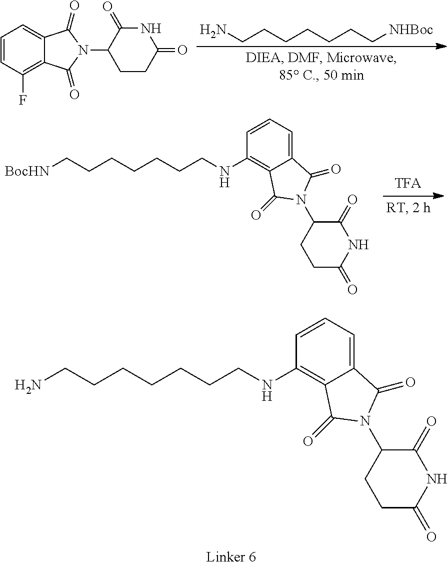 [SMILES: O=C1CCC(N2C(=O)c3cccc(F)c3C2=O)C(=O)N1 ; CC(C)(C)OC(=O)NCCCCCCCN ; CC(C)(C)OC(=O)NCCCCCCCNc1cccc2c1C(=O)N(C1CCC(=O)NC1=O)C2=O ; NCCCCCCCNc1cccc2c1C(=O)N(C1CCC(=O)NC1=O)C2=O]

Linker 6 was synthesized following the same procedures as Linker 1 as described for Example 1. (2.0 g, yield: 94%). ¹H NMR (400 MHz, DMSO-d₆) δ 11.05 (br, 1H), 7.94-7.56 (m, 4H), 7.10-7.02 (m, 2H), 6.52 (t, J=6.0 Hz, 1H), 5.07-5.02 (m, 1H), 3.32-3.27 (m, 2H), 2.88-2.77 (m, 1H), 2.75-2.61 (m, 2H), 2.60-2.50 (m, 2H), 2.04-2.02 (m, 1H), 1.59-1.50 (m, 4H), 1.35-1.30 (m, 6H). MS (ESI) m/z=387.2 [M+H]⁺.

### Example 7: 4-((8-Aminooctyl)amino)-2-(2,6-dioxopiperidin-3-yl)isoindoline-1,3-dione (Linker 7)

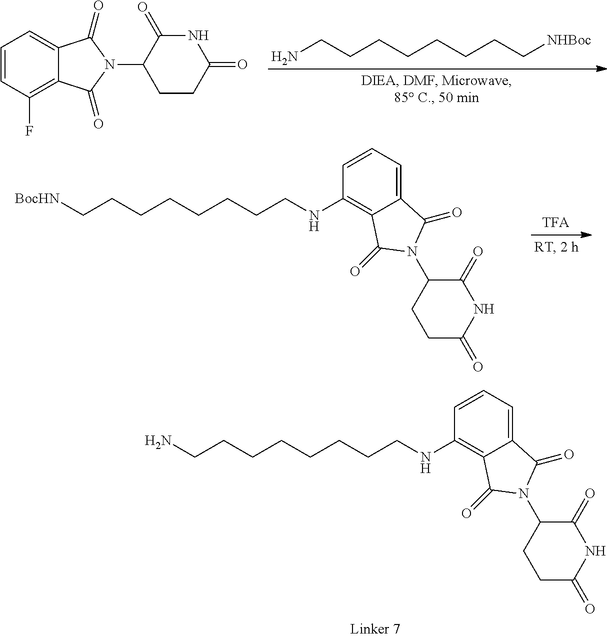

Linker 7 was synthesized following the same procedures as Linker 1 as described for Example 1. (1.1 g, yield: 61%). ¹H NMR (400 MHz, DMSO-d₆) δ 11.10 (s, 1H), 7.69-7.56 (m, 4H), 7.09 (d, J=8.4 Hz, 1H), 7.03 (d, J=6.8 Hz, 1H), 6.52 (t, J=6.0 Hz, 1H), 5.07-5.03 (m, 1H), 3.34-3.26 (m, 2H), 2.89-2.85 (m, 1H), 2.76 (s, 2H), 2.61-2.56 (m, 2H), 2.04-2.00 (m, 1H), 1.59-1.49 (m, 4H), 1.35-1.27 (m, 8H). MS (ESI) m/z=401.2 [M+H]⁺.

### Example 8: 4-((2-(2-Aminoethoxy)ethyl)amino)-2-(2,6-dioxopiperidin-3-yl)isoindoline-1,3-dione (Linker 8)

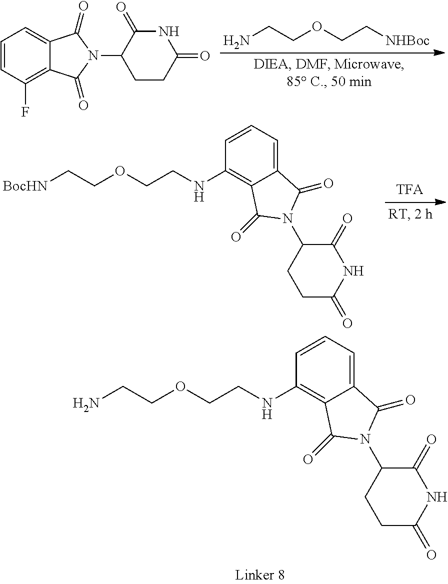

Linker 8 was synthesized following the same procedures as Linker 1 as described for Example 1. (2.0 g, yield: 94%). ¹H NMR (400 MHz, DMSO-d₆) δ 10.10 (s, 1H), 7.88 (s, 3H), 7.60 (t, J=8.0 Hz, 1H), 7.17 (d, J=8.4 Hz, 1H), 7.06 (d, J=6.8 Hz, 1H), 6.40 (d, J=5.6 Hz, 1H), 5.05 (dd, J=5.2, 12.8 Hz, 1H), 3.67-3.62 (m, 4H), 3.54-3.50 (m, 2H), 3.00 (s, 2H), 2.90-2.85 (m, 1H), 2.62-2.50 (m, 2H), 2.03 (t, J=7.6 Hz, 1H). MS (ESI) m/z=361.1 [M+H]⁺.

### Example 9: 4-((2-(2-(2-Aminoethoxy)ethoxy)ethyl)amino)-2-(2,6-dioxopiperidin-3-yl)isoindoline-1,3-dione (Linker 9)

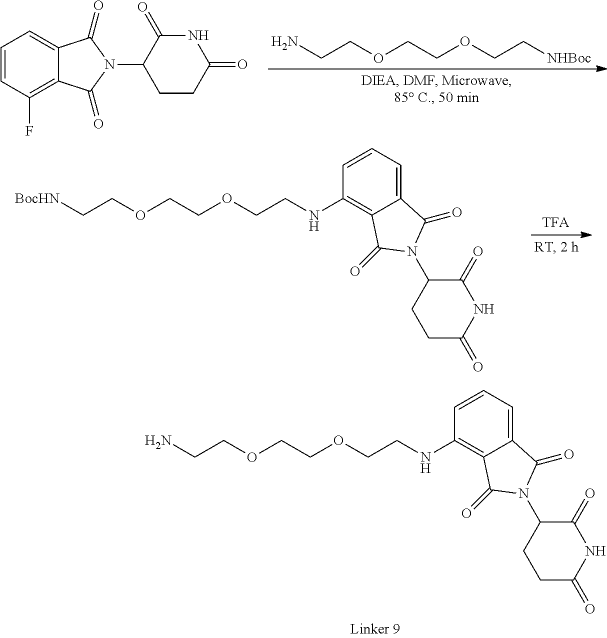

Linker 9 was synthesized following the same procedures as Linker 1 as described for Example 1. (1.1 g, yield: 82%). ¹H NMR (400 MHz, DMSO-d₆) δ 11.11 (s, 1H), 7.84 (s, 3H), 7.62-7.58 (m, 1H), 7.15 (d, J=8.8 Hz, 1H), 7.05 (d, J=6.8 Hz, 1H), 6.62-6.59 (m, 1H), 5.08-5.04 (m, 1H), 3.65-3.59 (m, 8H), 3.50-3.46 (m, 2H), 2.97-2.86 (m, 3H), 2.62-2.51 (m, 2H), 2.05-1.99 (m, 1H). MS (ESI) m/z=405.2 [M+H]⁺.

### Example 10: 4-((2-(2-(2-(2-Aminoethoxy)ethoxy)ethoxy)ethyl)amino)-2-(2,6-dioxopiperidin-3-yl)isoindoline-1,3-dione (Linker 10)

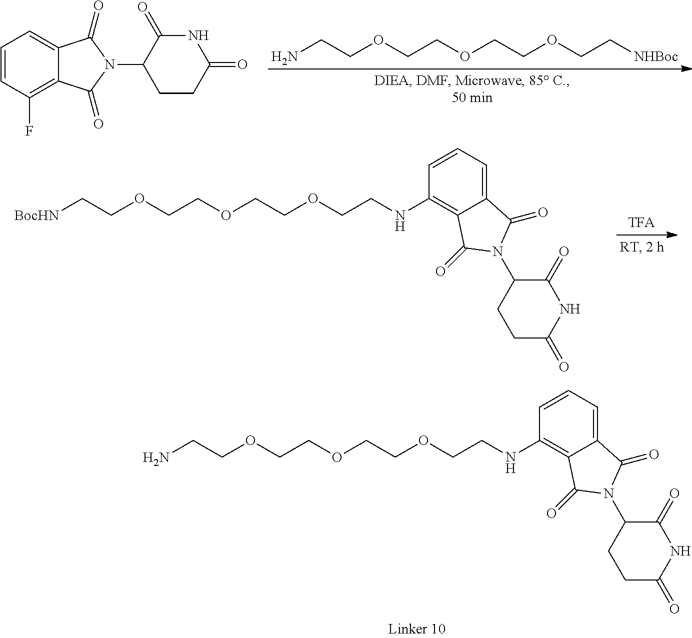

Linker 10 was synthesized following the same procedures as Linker 1 as described for Example 1. (1.3 g, yield: 70%). ¹H NMR (400 MHz, DMSO-d₆) δ 11.11 (s, 1H), 7.83 (s, 3H), 7.61-7.57 (m, 1H), 7.15 (d, J=8.8 Hz, 1H), 7.05 (d, J=6.8 Hz, 1H), 6.62-6.59 (m, 1H), 5.08-5.04 (m, 1H), 3.64-3.45 (m, 14H), 2.97-2.86 (m, 3H), 2.62-2.51 (m, 2H), 2.08-2.01 (m, 1H). MS (ESI) m/z=449.2 [M+H]⁺.

### Example 11: 4-((14-Amino-3,6,9,12-tetraoxatetradecyl)amino)-2-(2,6-dioxopiperidin-3-yl)isoindoline-1,3-dione (Linker 11)

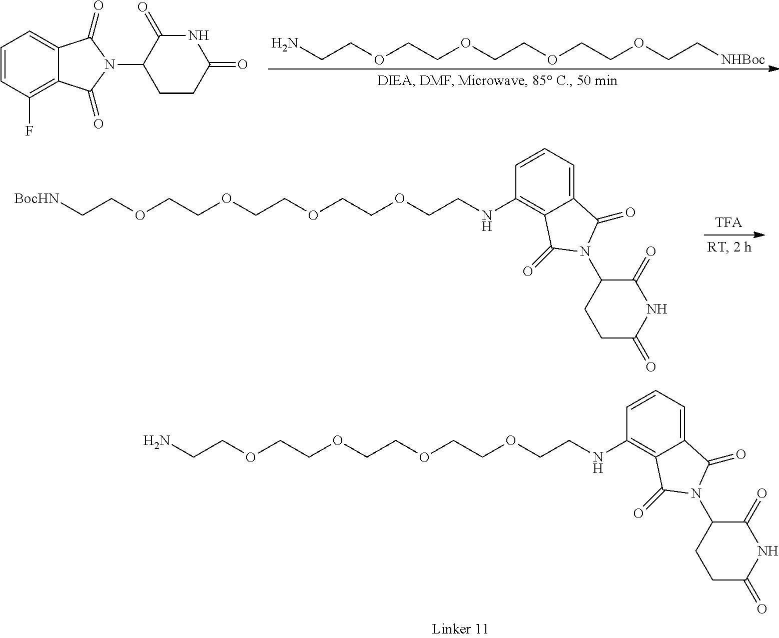

Linker 11 was synthesized following the same procedures as Linker 1 as described for Example 1. (1.2 g, yield: 89%). ¹H NMR (400 MHz, DMSO-d₆) δ 11.11 (s, 1H), 7.84 (s, 3H), 7.61-7.57 (m, 1H), 7.15 (d, J=8.8 Hz, 1H), 7.05 (d, J=6.8 Hz, 1H), 6.61 (s, 1H), 5.08-5.04 (m, 1H), 3.64-3.47 (m, 18H), 2.99-2.86 (m, 3H), 2.62-2.51 (m, 2H), 2.08-2.01 (m, 1H). MS (ESI) m/z=493.2 [M+H]⁺.

### Example 12: 4-((17-Amino-3,6,9,12,15-pentaoxaheptadecyl)amino)-2-(2,6-dioxopiperidin-3-yl)isoindoline-1,3-dione (Linker 12)

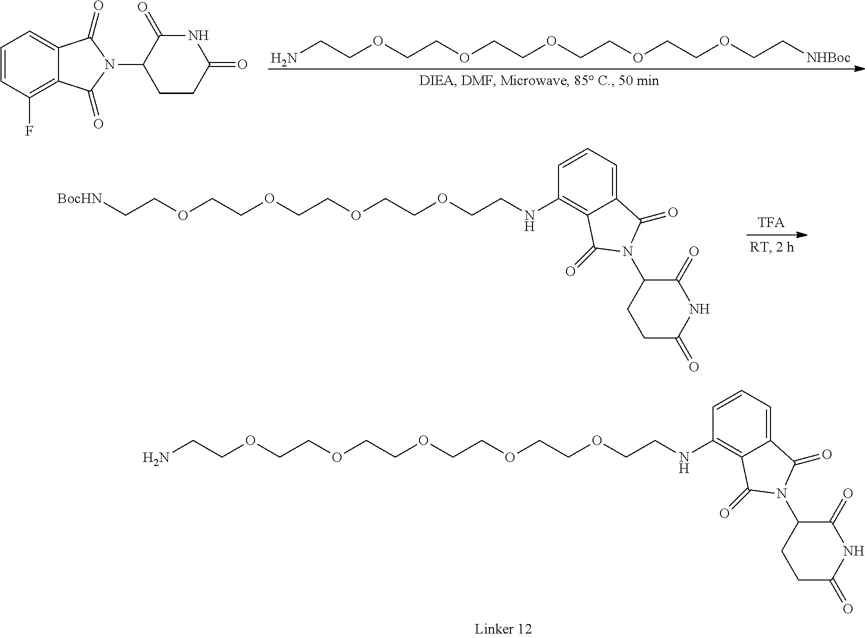

Linker 12 was synthesized following the same procedures as Linker 1 as described for Example 1. (1.2 g, yield: 86%). ¹H NMR (400 MHz, DMSO-d₆) δ 11.11 (s, 1H), 7.82 (s, 3H), 7.61-7.57 (m, 1H), 7.15 (d, J=8.4 Hz, 1H), 7.05 (d, J=7.2 Hz, 1H), 6.61-6.59 (m, 1H), 5.08-5.03 (m, 1H), 3.64-3.47 (m, 22H), 3.00-2.86 (m, 3H), 2.62-2.51 (m, 2H), 2.05-2.02 (m, 1H). MS (ESI) m/z=537.2 [M+H]⁺.

### Example 13: (2-(2,6-Dioxopiperidin-3-yl)-1,3-dioxoisoindolin-4-yl)glycine (Linker 13)

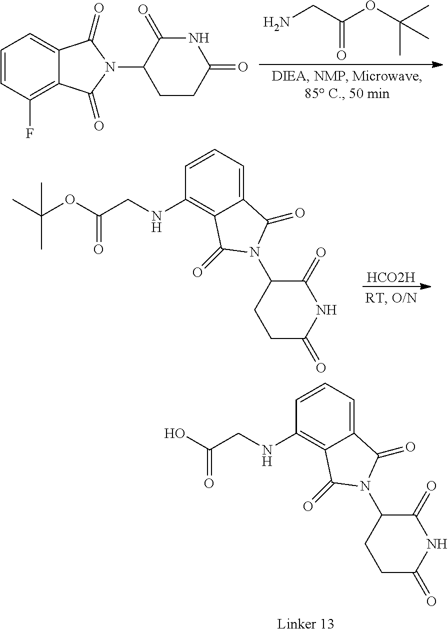

Linker 13 was synthesized following the same procedures as Linker 1 as described for Example 1. (840 mg, yield: 98%). ¹H NMR (400 MHz, DMSO-d₆) δ 11.07 (s, 1H), 7.52 (t, J=7.6 Hz, 1H), 6.99-6.88 (m, 3H), 5.04 (dd, J=5.2, 12.8 Hz, 1H), 3.73 (s, 2H), 2.93-2.83 (m, 1H), 2.61-2.50 (m, 2H), 2.02 (t, J=5.6 Hz, 1H). MS (ESI) m/z=330.1 [M−H]⁻.

### Example 14: 3-((2-(2,6-Dioxopiperidin-3-yl)-1,3-dioxoisoindolin-4-yl)amino)propanoic acid (Linker 14)

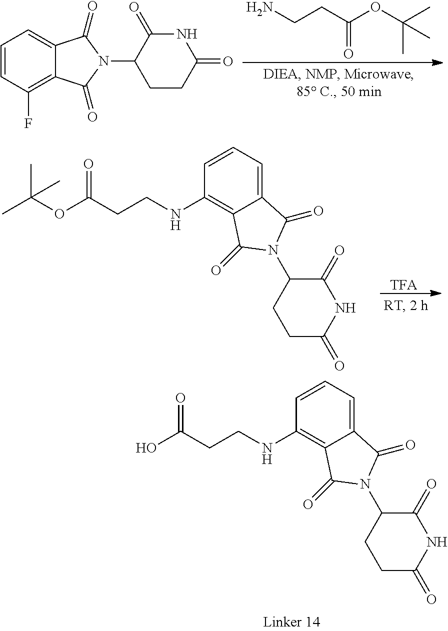

Linker 14 was synthesized following the same procedures as Linker 1 as described for Example 1. (1.42 g, yield: 88%). ¹H NMR (400 MHz, DMSO-d₆) δ 11.61 (br, 1H), 11.08 (s, 1H), 7.58 (dd, J=7.2, 8.8 Hz, 1H), 7.15 (d, J=8.8 Hz, 1H), 7.04 (d, J=7.2 Hz, 1H), 6.64 (s, 1H), 5.05 (dd, J=5.2, 12.8 Hz, 1H), 3.53 (t, J=6.4 Hz, 2H), 2.92-2.83 (m, 1H), 2.61-2.50 (m, 4H), 2.05-2.00 (m, 1H). MS (ESI) m/z=346.1 [M+H]⁺.

### Example 15: 4-((2-(2,6-Dioxopiperidin-3-yl)-1,3-dioxoisoindolin-4-yl)amino)butanoic acid (Linker 15)

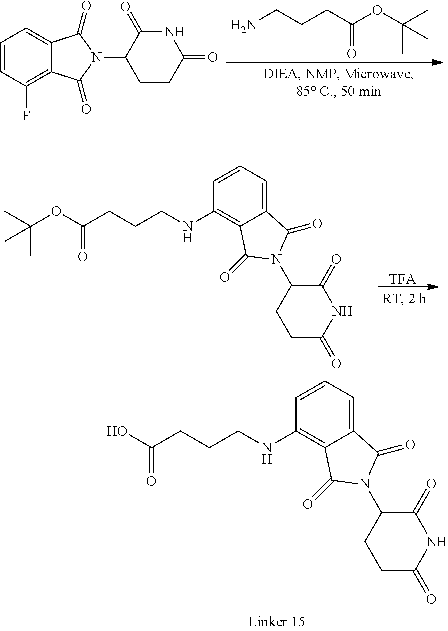

Linker 15 was synthesized following the same procedures as Linker 1 as described for Example 1. (1.27 g, yield: 53%). ¹H NMR (400 MHz, DMSO-d₆) δ 12.12 (br, 1H), 11.08 (s, 1H), 7.58 (dd, J=7.2, 8.8 Hz, 1H), 7.13 (d, J=8.8 Hz, 1H), 7.03 (d, J=7.2 Hz, 1H), 6.64 (t, J 10=6.0 Hz, 1H), 5.05 (dd, J=5.6, 12.8 Hz, 1H), 3.33 (q, J=6.8 Hz, 2H), 2.93-2.83 (m, 1H), 2.61-2.50 (m, 2H), 2.31 (t, J=6.8 Hz, 2H), 2.07-2.00 (m, 1H), 1.83-1.75 (m, 2H). MS (ESI) m/z=360.1 [M+H]⁺.

### Example 16: 5-((2-(2,6-Dioxopiperidin-3-yl)-1,3-dioxoisoindolin-4-yl)amino)pentanoic acid (Linker 16)

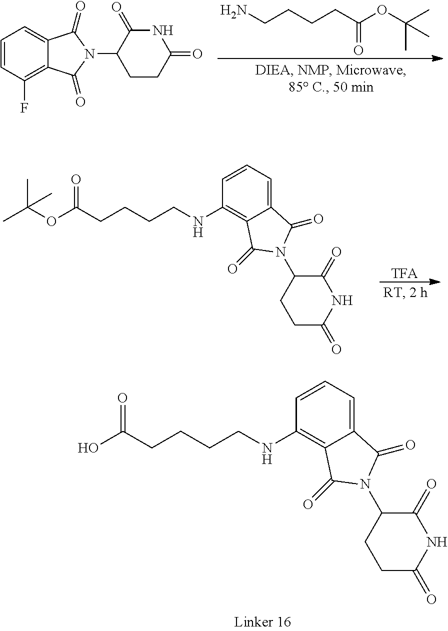

Linker 16 was synthesized following the same procedures as Linker 1 as described for Example 1. (1.4 g, yield: 85%). ¹H NMR (400 MHz, DMSO-d₆) δ 12.02 (br, 1H), 11.08 (s, 1H), 7.58 (dd, J=8.8, 7.2 Hz, 1H), 7.10 (d, J=8.4 Hz, 1H), 7.02 (d, J=7.2 Hz, 1H), 6.64 (t, J=5.6 Hz, 1H), 5.07-5.03 (m, 1H), 3.32-3.02 (m, 2H), 2.93-2.84 (m, 1H), 2.61-2.54 (m, 2H), 2.28-2.25 (m, 2H), 2.05-2.01 (m, 1H), 1.60-1.51 (m, 4H). MS (ESI) m/z=374.1 [M+H]⁺.

### Example 17: 6-((2-(2,6-Dioxopiperidin-3-yl)-1,3-dioxoisoindolin-4-yl)amino)hexanoic acid (Linker 17)

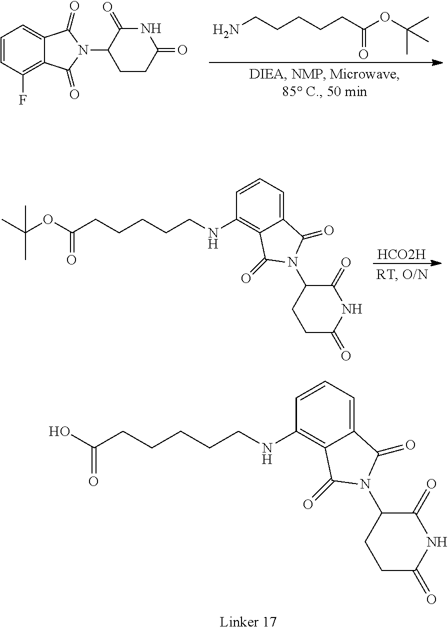

Linker 17 was synthesized following the same procedures as Linker 1 as described for Example 1. (1.43 g, yield: 91%). ¹H NMR (400 MHz, DMSO-d₆) δ 11.97 (s, 1H), 11.08 (s, 1 H), 7.57 (dd, J=7.2, 8.8 Hz, 1H), 7.08 (d, J=8.8 Hz, 1H), 7.02 (d, J=7.2 Hz, 1H), 6.52 (t, J=6.0 Hz, 1H), 5.05 (dd, J=5.6, 12.8 Hz, 1H), 3.30 (q, J=6.8 Hz, 2H), 2.93-2.83 (m, 1H), 2.61-2.50 (m, 2H), 2.32 (t, J=7.2 Hz, 2H), 2.07-2.00 (m, 1H), 1.61-1.50 (m, 4H), 1.39-1.33 (m, 2H). MS (ESI) m/z=388.1 [M+H]⁺.

### Example 18: 7-((2-(2,6-Dioxopiperidin-3-yl)-1,3-dioxoisoindolin-4-yl)amino)heptanoic acid (Linker 18)

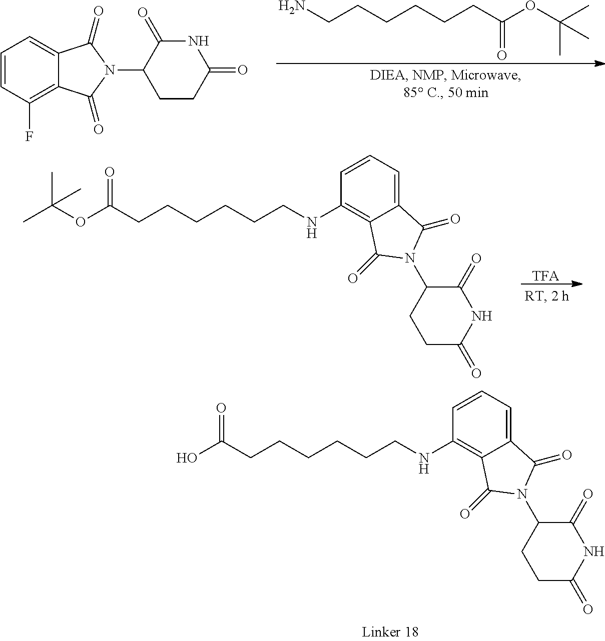

Linker 18 was synthesized following the same procedures as Linker 1 as described for Example 1. (2.3 g, yield: 86%). ¹H NMR (400 MHz, DMSO-d₆) δ 11.92 (br, 1H), 11.08 (s, 1H), 7.57 (t, J=8.0 Hz, 1H), 7.13 (d, J=8.8 Hz, 1H), 7.03 (d, J=6.8 Hz, 1H), 6.52 (t, J=5.6 Hz, 1H), 5.05 (dd, J=5.6, 12.8 Hz, 1H), 3.30 (q, J=6.4 Hz, 2H), 2.93-2.83 (m, 1H), 2.61-2.50 (m, 2H), 2.31 (t, J=7.2 Hz, 2H), 2.07-2.00 (m, 1H), 1.58-1.48 (m, 4H), 1.34-1.31 (m, 4H). MS (ESI) m/z=402.1 [M+H]⁺.

### Example 19: 8-((2-(2,6-Dioxopiperidin-3-yl)-1,3-dioxoisoindolin-4-yl)amino)octanoic acid (Linker 19)

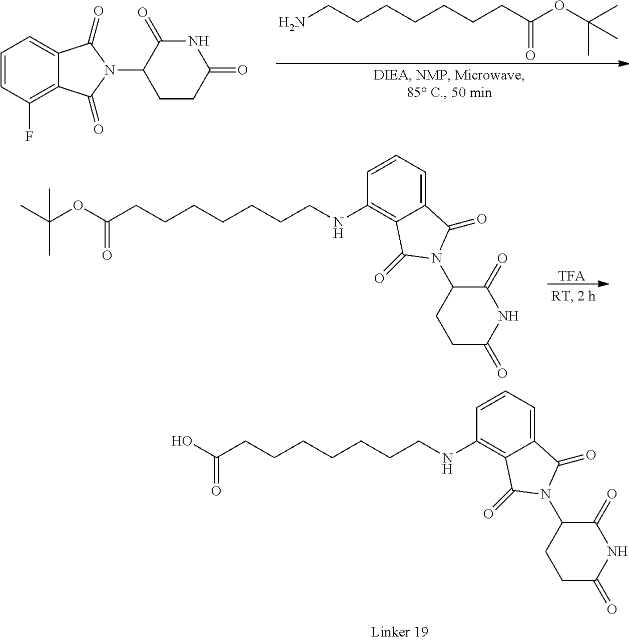

Linker 19 was synthesized following the same procedures as Linker 1 as described for Example 1. (1.14 g, yield: 77%). ¹H NMR (400 MHz, DMSO-d₆) δ 11.94 (s, 1H), 11.08 (s, 1H), 7.57 (t, J=8.0 Hz, 1H), 7.08 (d, J=8.4 Hz, 1H), 7.02 (d, J=6.8 Hz, 1H), 6.52 (t, J=5.6 Hz, 1H), 5.05 (dd, J=5.6, 12.8 Hz, 1H), 3.31-3.26 (m, 2H), 2.93-2.83 (m, 1H), 2.61-2.50 (m, 2H), 2.19 (t, J=7.2 Hz, 2H), 2.05-2.00 (m, 1H), 1.58-1.47 (m, 4H), 1.35-1.25 (s, 6H). MS (ESI) m/z=416.1 [M+H]⁺.

### Example 20: 3-(2-((2-(2,6-Dioxopiperidin-3-yl)-1,3-dioxoisoindolin-4-yl)amino)ethoxy)propanoic acid (Linker 20)

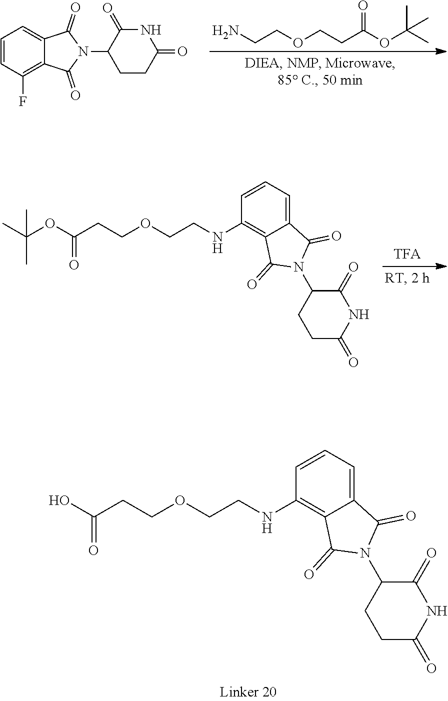 [SMILES: O=C1CCC(N2C(=O)c3cccc(F)c3C2=O)C(=O)N1 ; CC(C)(C)OC(=O)CCOCCN ; CC(C)(C)OC(=O)CCOCCNc1cccc2c1C(=O)N(C1CCC(=O)NC1=O)C2=O ; O=C(O)CCOCCNc1cccc2c1C(=O)N(C1CCC(=O)NC1=O)C2=O]

Linker 20 was synthesized following the same procedures as Linker 1 as described for Example 1. (3.5 g, yield: 80%). ¹H NMR (400 MHz, DMSO-d₆) δ 12.18 (s, 1H), 11.08 (s, 1H), 7.58 (dd, J=7.2 Hz, 8.8 Hz, 1H), 7.13 (d, J=8.4 Hz, 1H), 7.04 (d, J=7.2 Hz, 1H), 6.58 (t, J=5.6 Hz 1H), 5.05 (dd, J=6.4 Hz, 12.8 Hz, 1H), 3.67-3.58 (m, 4H), 3.47-3.43 (m, 2H), 2.93-2.84 (m, 1H), 2.61-2.45 (m, 4H), 2.07-2.01 (m, 1H). MS (ESI) m/z=390.1 [M+H]⁺.

### Example 21: 3-(2-(2-((2-(2,6-Dioxopiperidin-3-yl)-1,3-dioxoisoindolin-4-yl)amino)ethoxy)ethoxy)propanoic acid (Linker 21)

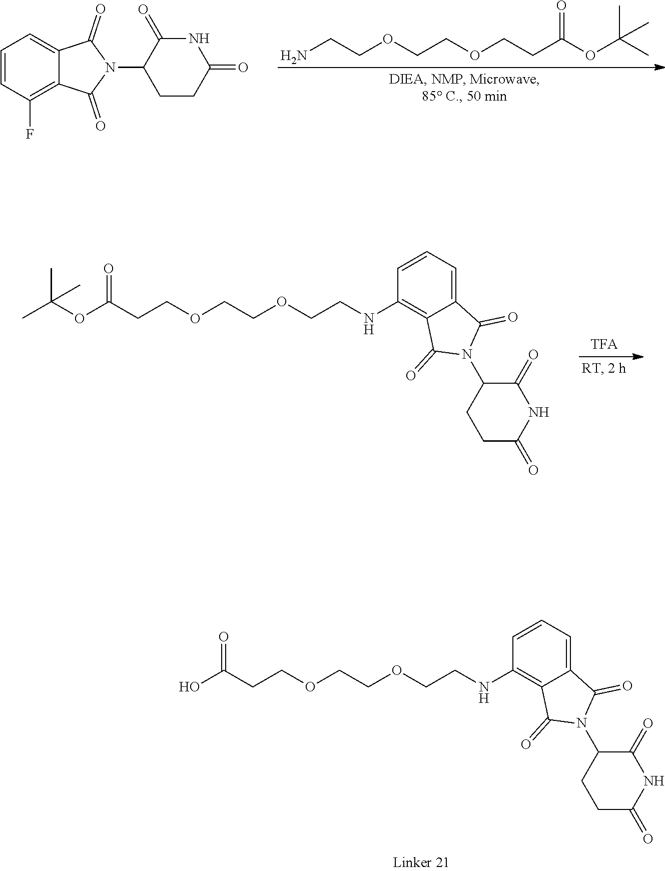 [SMILES: O=C1CCC(N2C(=O)c3cccc(F)c3C2=O)C(=O)N1 ; CC(C)(C)OC(=O)CCOCCOCCN ; CC(C)(C)OC(=O)CCOCCOCCNc1cccc2c1C(=O)N(C1CCC(=O)NC1=O)C2=O ; O=C(O)CCOCCOCCNc1cccc2c1C(=O)N(C1CCC(=O)NC1=O)C2=O]

Linker 21 was synthesized following the same procedures as Linker 1 as described for Example 1. (2.0 g, yield: 58%). ¹H NMR (400 MHz, DMSO-d₆) δ 12.13 (s, 1H), 11.08 (s, 1H), 7.58 (dd, J=7.2 Hz, 8.4 Hz, 1H), 7.14 (d, J=8.4 Hz, 1H), 7.04 (d, J=6.8 Hz, 1H), 6.60 (t, J=6.0 Hz 1H), 5.05 (dd, J=5.2 Hz, 12.4 Hz, 1H), 3.63-3.44 (m, 10H), 2.88-2.85 (m, 1H), 2.61-2.49 (m, 2H), 2.44-2.41 (m, 2H), 2.04-2.01 (m, 1H). MS (ESI) m/z=434.1 [M+H]⁺.

### Example 22: 3-(2-(2-(2-((2-(2,6-Dioxopiperidin-3-yl)-1,3-dioxoisoindolin-4-yl)amino)ethoxy)ethoxy)ethoxy)propanoic acid (Linker 22)

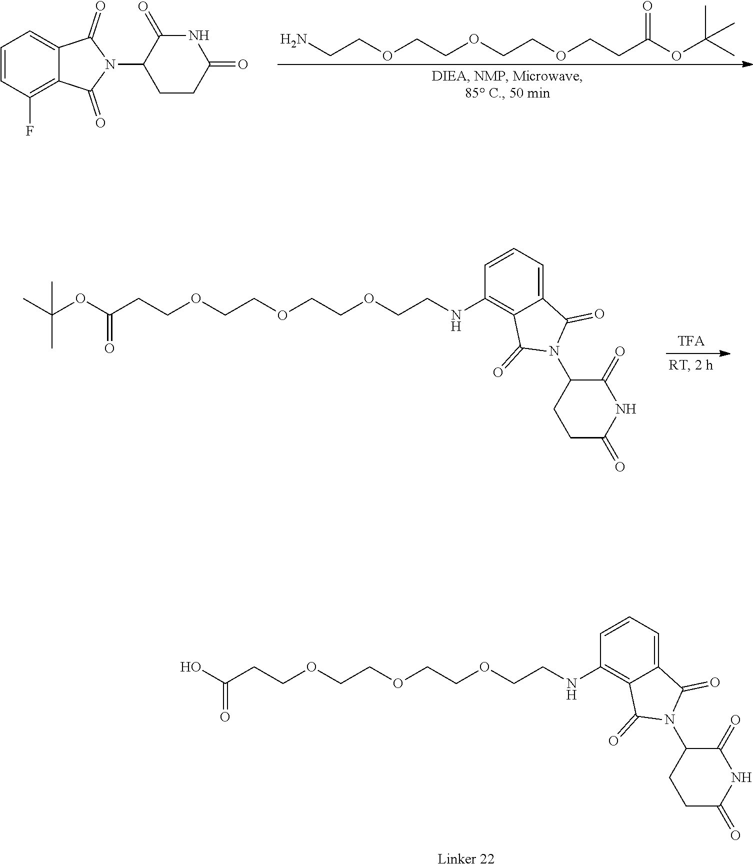 [SMILES: O=C1CCC(N2C(=O)c3cccc(F)c3C2=O)C(=O)N1 ; CC(C)(C)OC(=O)CCOCCOCCOCCN ; CC(C)(C)OC(=O)CCOCCOCCOCCNc1cccc2c1C(=O)N(C1CCC(=O)NC1=O)C2=O ; O=C(O)CCOCCOCCOCCNc1cccc2c1C(=O)N(C1CCC(=O)NC1=O)C2=O]

Linker 22 was synthesized following the same procedures as Linker 1 as described for Example 1. (3.2 g, yield: 93%). ¹H NMR (400 MHz, DMSO-d₆) δ 12.14 (s, 1H), 11.08 (s, 1H), 7.58 (dd, J=7.2 Hz, 8.4 Hz, 1H), 7.14 (d, J=8.8 Hz, 1H), 7.04 (d, J=6.8 Hz, 1H), 6.60 (t, J=6.0 Hz, 1H), 5.05 (dd, J=5.2 Hz, 12.8 Hz, 1H), 3.63-3.45 (m, 14H), 2.88-2.85 (m, 1H), 2.61-2.49 (m, 2H), 2.44-2.40 (m, 2H), 2.04-2.01 (m, 1H). MS (ESI) m/z=478.2 [M+H]⁺.

### Example 23: 1-((2-(2,6-Dioxopiperidin-3-yl)-1,3-dioxoisoindolin-4-yl)amino)-3,6,9,12-tetraoxapentadecan-15-oic acid (Linker 23)

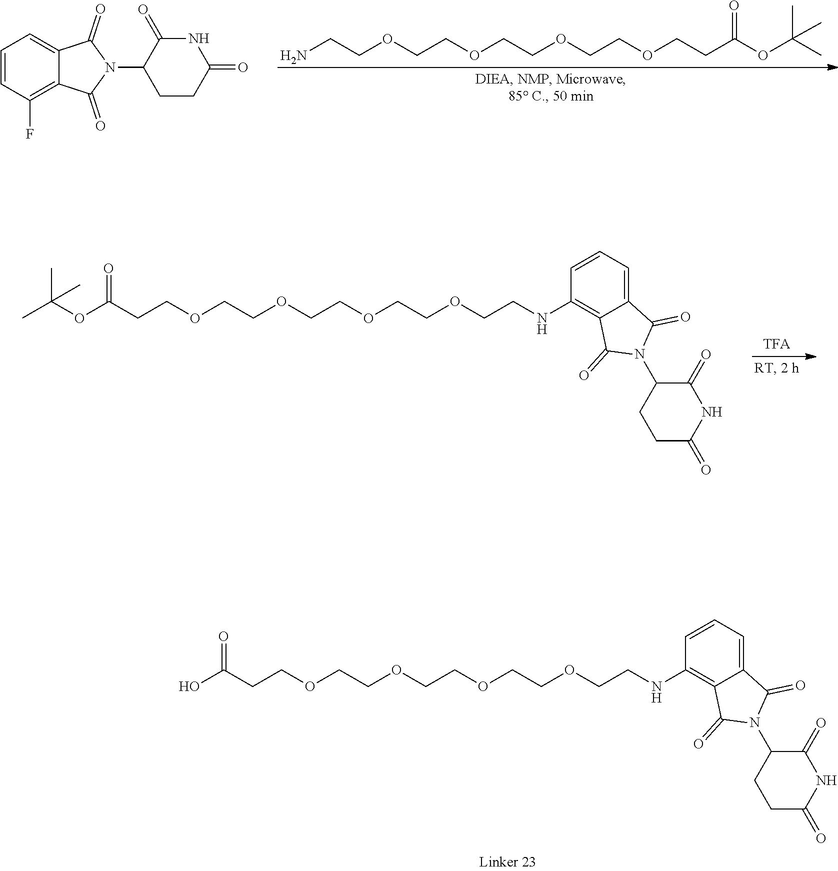

Linker 23 was synthesized following the same procedures as Linker 1 as described for Example 1. (2.3 g, yield: 59%). ¹H NMR (400 MHz, DMSO-d₆) δ 12.14 (s, 1H), 11.08 (s, 1H), 7.58 (dd, J=7.2 Hz, 8.8 Hz, 1H), 7.14 (d, J=8.4 Hz, 1H), 7.04 (d, J=7.2 Hz, 1H), 6.60 (t, J=6.0 Hz, 1H), 5.05 (dd, J=5.2 Hz, 12.8 Hz, 1H), 3.63-3.48 (m, 18H), 2.898-2.85 (m, 1H), 2.61-2.49 (m, 2H), 2.44-2.41 (m, 2H), 2.04-2.01 (m, 1H). MS (ESI) m/z=522.2 [M+H]⁺.

### Example 24: 1-((2-(2,6-Dioxopiperidin-3-yl)-1,3-dioxoisoindolin-4-yl)amino)-3,6,9,12,15-pentaoxaoctadecan-18-oic acid (Linker 24)

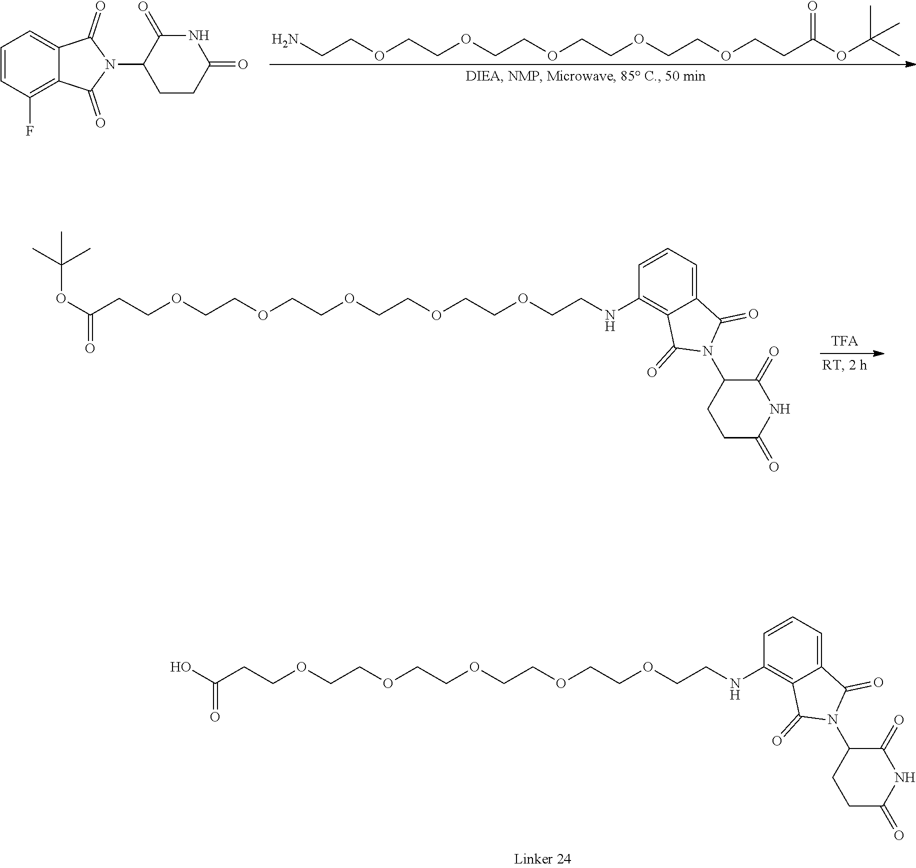

Linker 24 was synthesized following the same procedures as Linker 1 as described for Example 1. (2.4 g, yield: 66%). ¹H NMR (400 MHz, DMSO-d₆) δ 11.09 (s, 1H), 7.58 (dd, J=7.2, 8.4 Hz, 1H), 7.13 (d, J=8.4 Hz, 1H), 7.04 (d, J=7.2 Hz, 1H), 6.60 (t, J=5.6 Hz, 1H), 5.05 (dd, J=5.6, 12.8 Hz, 1H), 3.64-3.46 (m, 22H), 2.93-2.83 (m, 1H), 2.61-2.50 (m, 2H), 2.44-2.40 (m, 2H), 2.02 (t, J=6.4 Hz, 1H). MS (ESI) m/z=566.2 [M+H]⁺.

### Example 25: (2S,4R)-1-((S)-2-(2-Aminoacetamido)-3,3-dimethylbutanoyl)-4-hydroxy-N-(4-(4-methylthiazol-5-yl)benzyl)pyrrolidine-2-carboxamide (Linker 25)

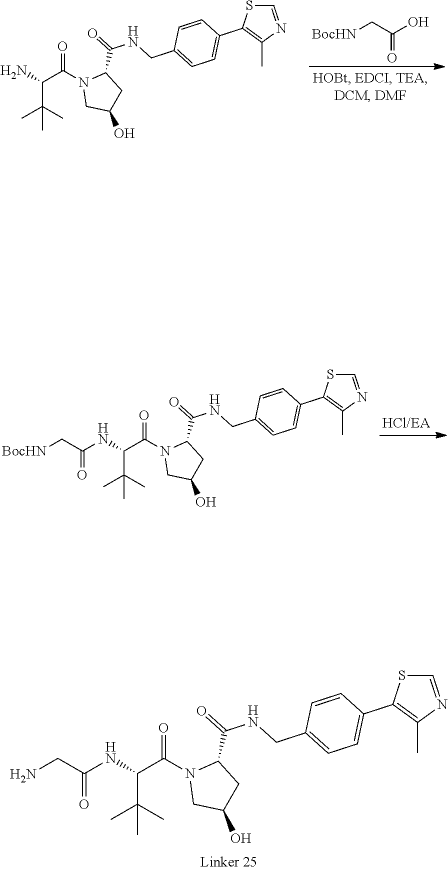 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCC(=O)O ; Cc1ncsc1-c1ccc(CNC(=O)[C@@H]2C[C@@H](O)CN2C(=O)[C@@H](NC(=O)CNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CN)C(C)(C)C)cc1]

Step 1: To a solution of (2S,4R)-1-((S)-2-amino-3,3-dimethylbutanoyl)-4-hydroxy-N-(4-(4-methylthiazol- 5-yl)benzyl)pyrrolidine-2-carboxamide (2.00 g, 4.67 mmol), 2-((tert-butoxycarbonyl)amino) acetic acid (900 mg, 5.14 mmol) and triethylamine (TEA) (3.2 mL, 23.35 mmol) in DCM/DMF (225 mL/11 mL) was added EDCI (1.07 g, 5.60 mmol), HOBt (756 mg, 5.60 mmol) at 0° C. The mixture was stirred at room temperature for 16 h, before the reaction mixture was poured into water and extracted with DCM. The combined organic layers were concentrated and the resulting residue was purified by chromatography on a silica gel column (DCM/MeOH=20/1, v/v) to give the desired product tert-butyl (2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethyl)carbamate (1.5 g, yield: 55%). MS (ESI) m/z=588.2 [M+H]⁺.

Step 2: To a solution of tert-butyl (2-(((S)-1-((2S,4R)-4-hydroxy-2-((4-(4-methylthiazol-5-yl) benzyl)carbamoyl)pyrrolidin-1-yl)-3,3-dimethyl-1-oxobutan-2-yl)amino)-2-oxoethyl)carbamate (1.50 g, 2.56 mmol) in ethylacetate (EtOAc) (30 mL) was added HCl/EtOAc (100 mL, 4 M). The mixture was stirred at room temperature for 3 h and filtered to give the desired product which was dissolved in water (100 mL) and lyophilized to give (2S,4R)-1-((S)-2-(2-aminoacetamido)-3,3-dimethylbutanoyl)-4-hydroxy-N-(4-(4-methylthiazol-5-yl)benzyl)pyrrolidine-2-carboxamide hydrochloride (Linker 25) (1.07 g, yield: 80%). ¹H NMR (400 MHz, DMSO-d₆) δ 9.29 (s, 1H), 8.72 (s, 1H), 8.56 (d, J=9.2 Hz, 1H), 8.26 (s, 3H), 7.38-7.47 (m, 4H), 4.61 (d, J=9.2 Hz, 1H), 4.36-4.47 (m, 3H), 4.20-4.25 (m, 1H), 3.60-3.70 (m, 4H), 2.46 (s, 3H), 2.10-2.05 (m, 1H), 1.97-1.89 (m, 1H), 0.95 (s, 9H). MS (ESI) m/z=488.3 [M+H]⁺.

### Example 26: (2S,4R)-1-((S)-2-(3-Aminopropanamido)-3,3-dimethylbutanoyl)-4-hydroxy-N-(4-(4-methylthiazol-5-yl)benzyl)pyrrolidine-2-carboxamide (Linker 26)

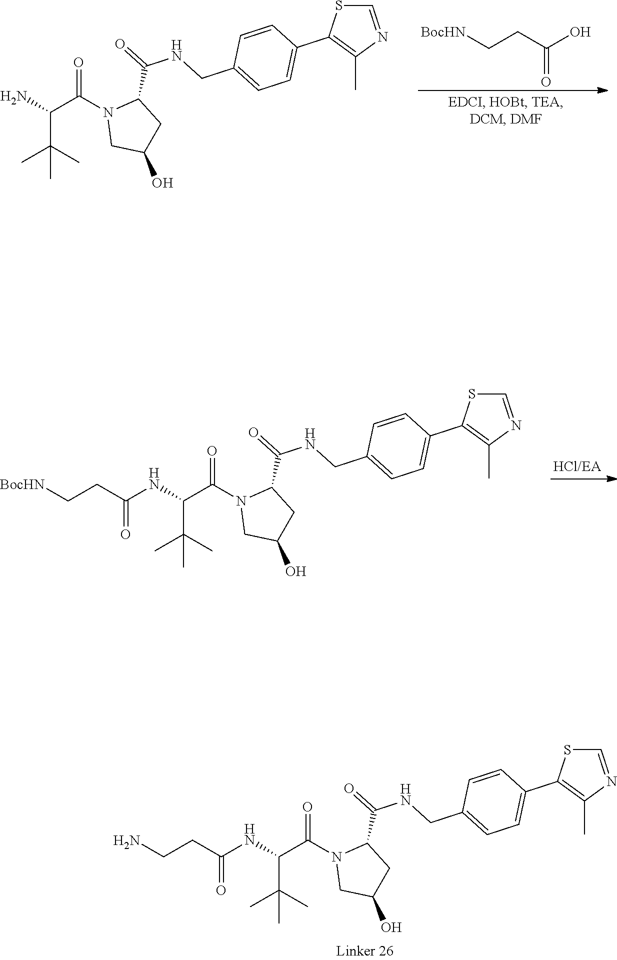 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCC(=O)O ; Cc1ncsc1-c1ccc(CNC(=O)[C@@H]2C[C@@H](O)CN2C(=O)[C@@H](NC(=O)CCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCN)C(C)(C)C)cc1]

Linker 26 was synthesized following the same procedures as Linker 25 as described for Example 25. (1.38 g, yield: 88%). ¹H NMR (400 MHz, DMSO-d₆) δ 9.36 (s, 1H), 8.68 (s, 1H), 8.26 (d, J=9.2 Hz, 1H), 8.16 (s, 3H), 7.49-7.39 (m, 4H), 4.53 (d, J=9.2 Hz, 1H), 4.47-4.35 (m, 3H), 4.24-4.19 (m, 1H), 3.69-3.60 (m, 2H), 2.94-2.93 (m, 2H), 2.64 (t, J=7.2 Hz, 2H), 2.48 (s, 3H), 2.06-2.01 (m, 1H), 1.92-1.85 (m, 1H), 0.95 (s, 9H). MS (ESI) m/z=502.3 [M+H]⁺.

### Example 27: (2S,4R)-1-((S)-2-(4-Aminobutanamido)-3,3-dimethylbutanoyl)-4-hydroxy-N-(4-(4-methylthiazol-5-yl)benzyl)pyrrolidine-2-carboxamide (Linker 27)

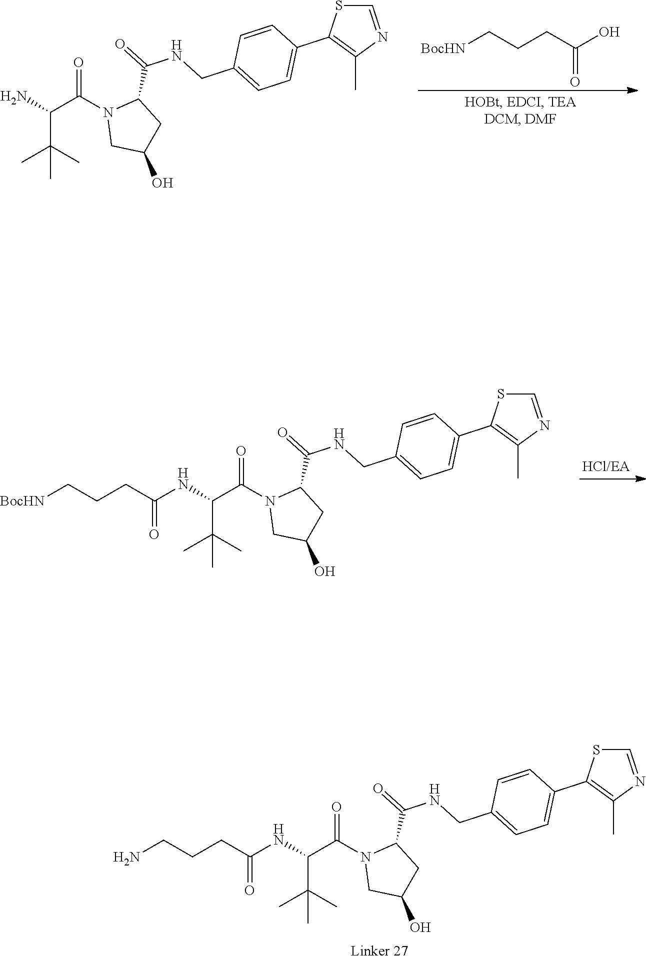 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCCC(=O)O ; Cc1ncsc1-c1ccc(CNC(=O)[C@@H]2C[C@@H](O)CN2C(=O)[C@@H](NC(=O)CCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCCN)C(C)(C)C)cc1]

Linker 27 was synthesized following the same procedures as Linker 25 as described for Example 25. (1.38 g, yield: 88%). ¹H NMR (400 MHz, DMSO-d₆) δ 9.66 (s, 1H), 8.74 (t, J=6.0, 1H), 8.25 (s, 3H), 8.03 (d, J=9.2 Hz, 1H), 7.49-7.41 (m, 4H), 4.53 (d, J=9.2 Hz, 1H), 4.51-4.35 (m, 3H), 4.29-4.24 (m, 1H), 3.71-3.65 (m, 2H), 2.79-2.77 (m, 2H), 2.52 (s, 3H), 2.45-2.27 (m, 2H), 2.12-2.07 (m, 1H), 1.94-1.80 (m, 3H), 0.94 (s, 9H). MS (ESI) m/z=516.0 [M+H]⁺.

### Example 28: (2S,4R)-1-((S)-2-(5-Aminopentanamido)-3,3-dimethylbutanoyl)-4-hydroxy-N-(4-(4-methylthiazol-5-yl)benzyl)pyrrolidine-2-carboxamide (Linker 28)

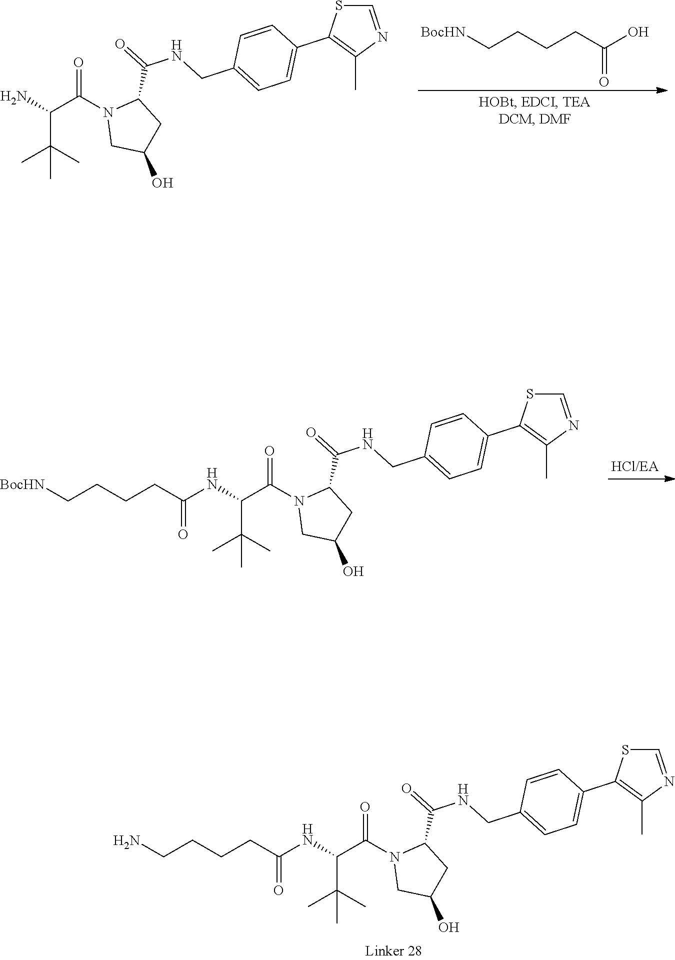 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCCCC(=O)O ; Cc1ncsc1-c1ccc(CNC(=O)[C@@H]2C[C@@H](O)CN2C(=O)[C@@H](NC(=O)CCCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCCCN)C(C)(C)C)cc1]

Linker 28 was synthesized following the same procedures as Linker 25 as described for Example 25. (1.50 g, yield: 79%). ¹H NMR (400 MHz, DMSO-d₆) δ 9.52 (s, 1H), 8.73 (t, J=11.6 Hz, 1H), 8.20 (s, 3H), 7.95 (d, J=9.6 Hz, 1H), 7.43-7.50 (m, 4H), 4.55 (d, J=9.2 Hz, 1H), 4.38-4.50 (m, 3H), 4.23-4.29 (m, 1H), 3.64-3.71 (m, 2H), 2.74-2.78 (m, 2H), 2.51 (s, 3H), 2.30-2.35 (m, 1H), 2.18-2.23 (m, 1H), 2.07-2.12 (m, 1H), 1.88-1.95 (m, 1H), 1.58 (d, J=4.4 Hz, 4H), 0.96 (s, 9H). MS (ESI) m/z=530.1 [M+H]⁺.

### Example 29: (2S,4R)-1-((S)-2-(6-Aminohexanamido)-3,3-dimethylbutanoyl)-4-hydroxy-N-(4-(4-methylthiazol-5-yl)benzyl)pyrrolidine-2-carboxamide (Linker 29)

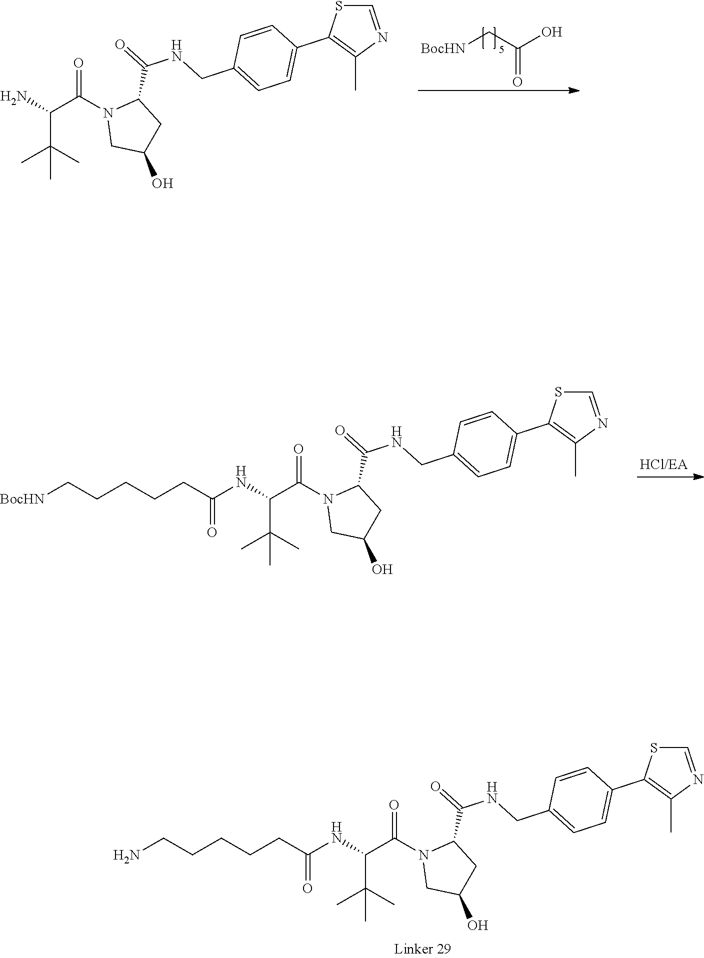

Linker 29 was synthesized following the same procedures as Linker 25 as described for Example 25. (2.70 g, yield: 98%). ¹H NMR (400 MHz, DMSO-d₆) δ 9.36 (s, 1H), 8.69 (t, J=6.4 Hz, 1H), 8.12 (brs, 3H), 7.92 (d, J=9.6 Hz, 1H), 7.44 (dd, J=13.6, 8.4 Hz, 4H), 4.54 (d, J=9.6 Hz, 1H), 4.48-4.39 (m, 2H), 4.36 (brs, 1H), 4.28-4.19 (m, 1H), 3.72-3.60 (m, 2H), 2.79-2.67 (m, 2H), 2.49 (s, 3H), 2.31-2.21 (m, 1H), 2.20-2.12 (m, 1H), 2.10-2.01 (m, 1H), 1.94-1.85 (m, 1H), 1.62-1.54 (m, 2H), 1.53-1.44 (m, 2H), 1.34-1.22 (m, 2H), 0.94 (s, 9H). MS (ESI) m/z=544.3 [M+H]⁺.

### Example 30: (2S,4R)-1-((S)-2-(7-Aminoheptanamido)-3,3-dimethylbutanoyl)-4-hydroxy-N-(4-(4-methylthiazol-5-yl)benzyl)pyrrolidine-2-carboxamide (Linker 30)

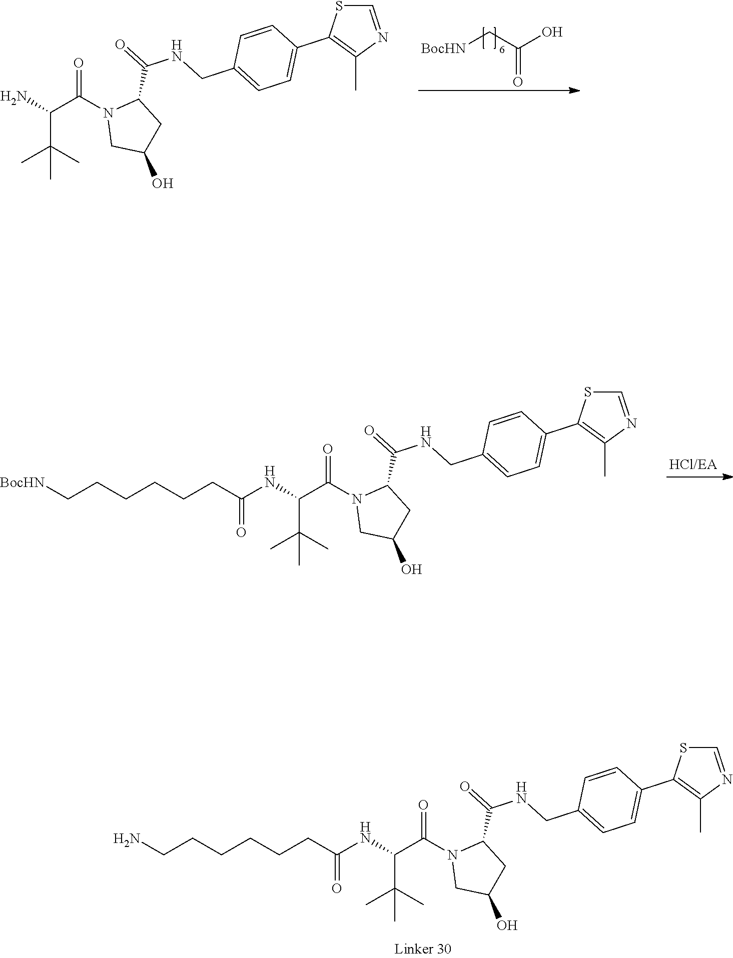

Linker 30 was synthesized following the same procedures as Linker 25 as described for Example 25. (2.13 g, yield: 86%). NH NMR (400 MHz, DMSO-d₆) δ 9.45 (s, 1H), 8.70 (t, J=6.0 Hz, 1H), 8.14 (brs, 3H), 7.86 (d, J=9.2 Hz, 1H), 7.44 (dd, J=12.8, 8.4 Hz, 4H), 4.54 (d, J=9.2 Hz, 1H), 4.49-4.40 (m, 2H), 4.36 (brs, 1H), 4.29-4.20 (m, 1H), 3.71-3.61 (m, 2H), 2.78-2.67 (m, 2H), 2.50 (s, 3H), 2.31-2.22 (m, 1H), 2.21-2.13 (m, 1H), 2.11-2.03 (m, 1H), 1.95-1.85 (m, 1H), 1.60-1.44 (m, 4H), 1.35-1.18 (m, 4H), 0.94 (s, 9H). MS (ESI) m/z=558.3 [M+H]⁺.

### Example 31: (2S,4R)-1-((S)-2-(8-Aminooctanamido)-3,3-dimethylbutanoyl)-4-hydroxy-N-(4-(4-methylthiazol-5-yl)benzyl)pyrrolidine-2-carboxamide (Linker 31)

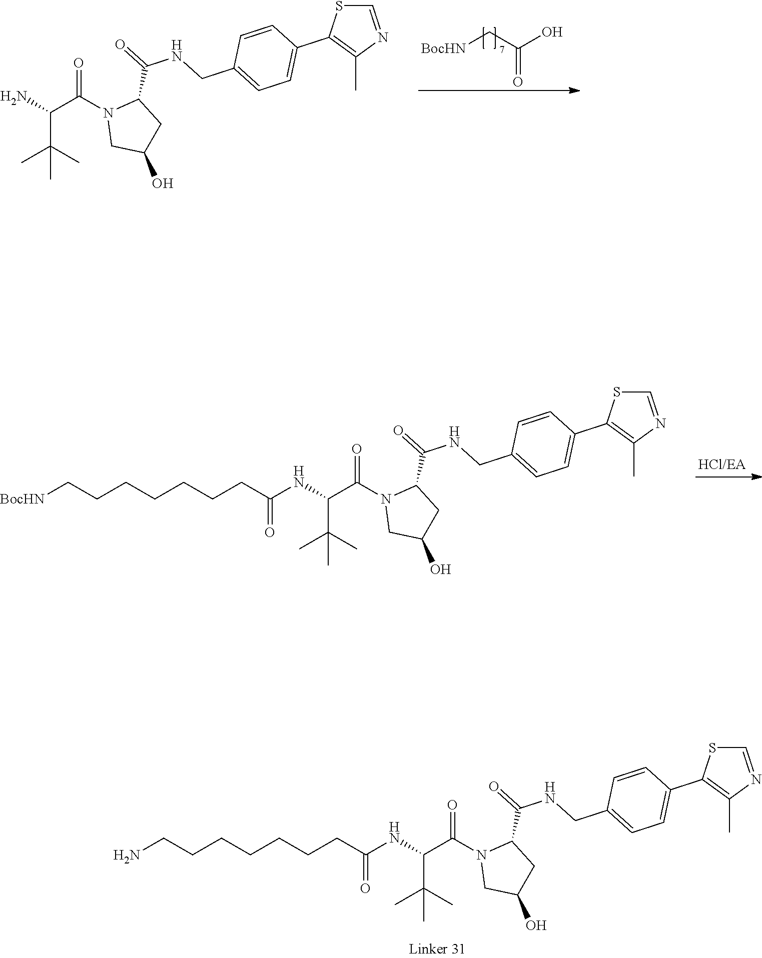

Linker 31 was synthesized following the same procedures as Linker 25 as described for Example 25. (1.81 g, yield: 72%). ¹H NMR (400 MHz, DMSO-d₆) δ 9.35 (s, 1H), 8.69 (t, J=6.0 Hz, 1H), 8.11 (brs, 3H), 7.88 (d, J=9.2 Hz, 1H), 7.44 (dd, J=14.0, 8.4 Hz, 4H), 4.54 (d, J=9.6 Hz, 1H), 4.48-4.39 (m, 2H), 4.36 (brs, 1H), 4.27-4.20 (m, 1H), 3.71-3.60 (m, 2H), 2.78-2.68 (m, 2H), 2.49 (s, 3H), 2.31-2.22 (m, 1H), 2.18-2.11 (m, 1H), 2.09-2.01 (m, 1H), 1.94-1.85 (m, 1H), 1.58-1.44 (m, 4H), 1.32-1.19 (m, 6H), 0.94 (s, 9H). MS (ESI) m/z=572.3 [M+H]⁺.

### Example 32: (2S,4R)-1-((S)-2-(9-Aminononanamido)-3,3-dimethylbutanoyl)-4-hydroxy-N-(4-(4-methylthiazol-5-yl)benzyl)pyrrolidine-2-carboxamide (Linker 32)

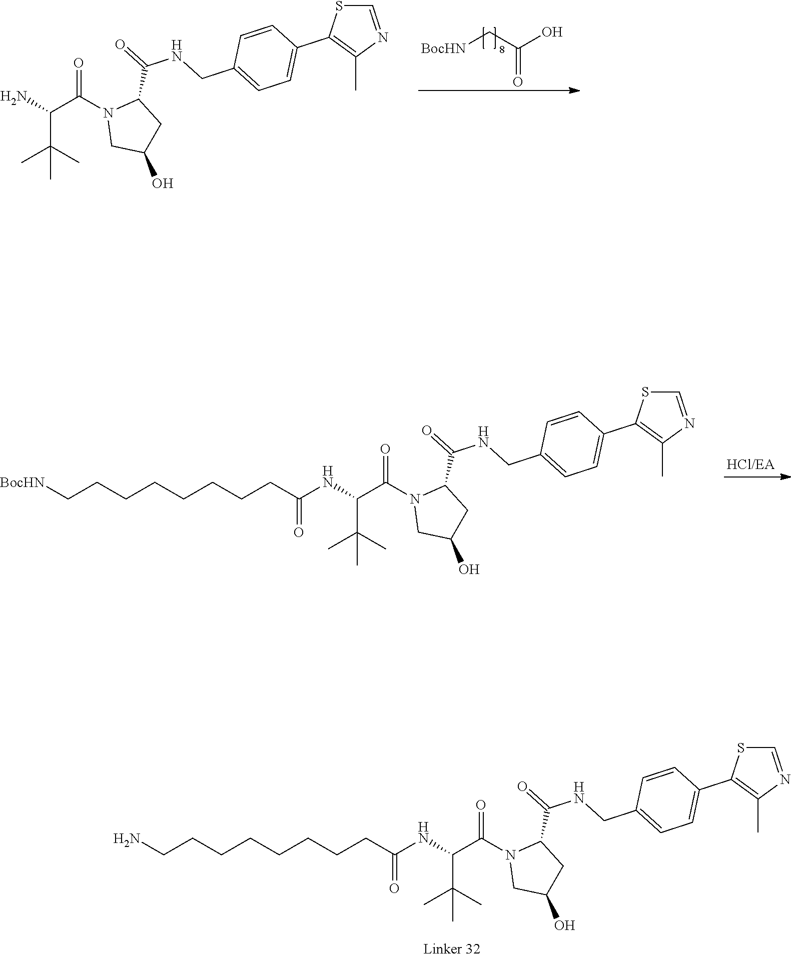

Linker 32 was synthesized following the same procedures as Linker 25 as described for Example 25. (2.32 g, yield: 89%). ¹H NMR (400 MHz, DMSO-d₆) δ 9.30 (s, 1H), 8.67 (t, J=6.4 Hz, 1H), 8.10 (brs, 3H), 7.88 (d, J=9.2 Hz, 1H), 7.43 (dd, J=14.0, 8.8 Hz, 4H), 4.55 (d, J=9.2 Hz, 1H), 4.48-4.39 (m, 2H), 4.35 (brs, 1H), 4.28-4.19 (m, 1H), 3.71-3.60 (m, 2H), 2.77-2.67 (m, 2H), 2.48 (s, 3H), 2.31-2.22 (m, 1H), 2.17-2.10 (m, 1H), 2.09-2.01 (m, 1H), 1.94-1.85 (m, 1H), 1.60-1.40 (m, 4H), 1.33-1.19 (m, 8H), 0.94 (s, 9H). m/z=586.3 [M+H]⁺.

### Example 33: (2S,4R)-1-((S)-2-(10-Aminodecanamido)-3,3-dimethylbutanoyl)-4-hydroxy-N-(4-(4-methylthiazol-5-yl)benzyl)pyrrolidine-2-carboxamide (Linker 33)

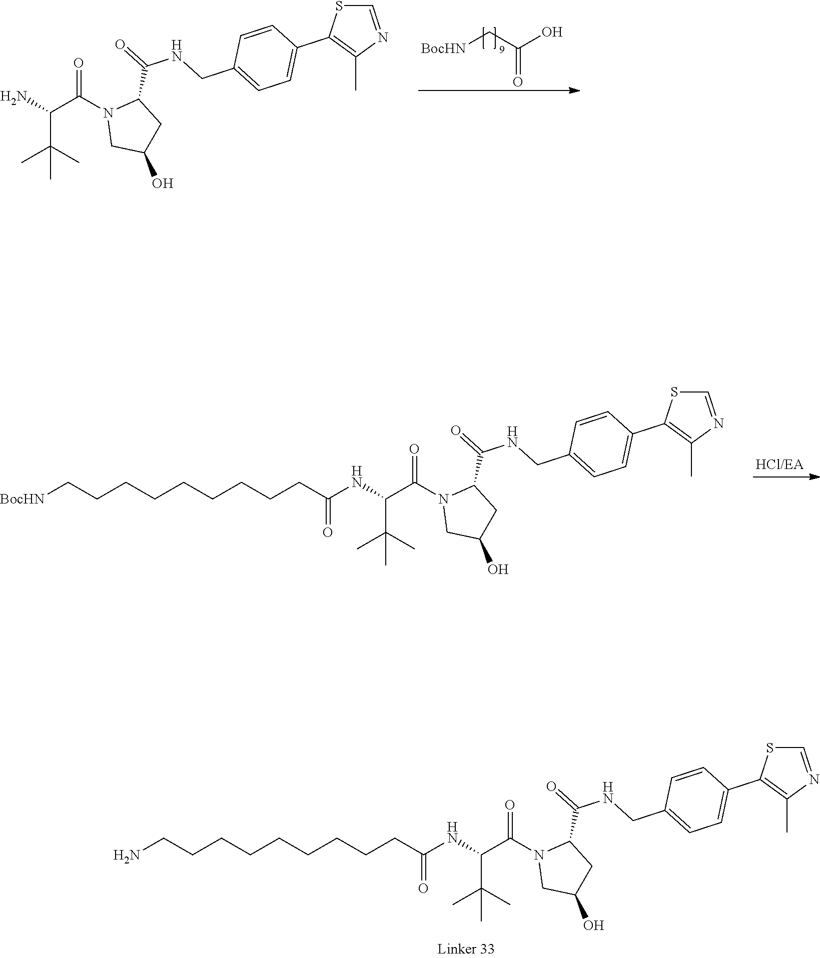

Linker 33 was synthesized following the same procedures as Linker 25 as described for Example 25. (2.29 g, yield: 90%). OH NMR (400 MHz, DMSO-d₆) δ 9.41 (s, 1H), 8.67 (t, J=6.0 Hz, 1H), 8.14 (brs, 3H), 7.85 (d, J=8.8 Hz, 1H), 7.44 (dd, J=13.6, 8.8 Hz, 4H), 4.54 (d, J=8.8 Hz, 1H), 4.48-4.39 (m, 2H), 4.36 (brs, 1H), 4.29-4.20 (m, 1H), 3.71-3.60 (m, 2H), 2.78-2.67 (m, 2H), 2.49 (s, 3H), 2.32-2.22 (m, 1H), 2.17-2.11 (m, 1H), 2.10-2.01 (m, 1H), 1.95-1.86 (m, 1H), 1.62-1.40 (m, 4H), 1.34-1.16 (m, 10H), 0.94 (s, 9H). MS (ESI) m/z=600.4 [M+H]⁺.

### Example 34: (2S,4R)-1-((S)-2-(11-Aminoundecanamido)-3,3-dimethylbutanoyl)-4-hydroxy-N-(4-(4-methylthiazol-5-yl)benzyl)pyrrolidine-2-carboxamide (Linker 34)

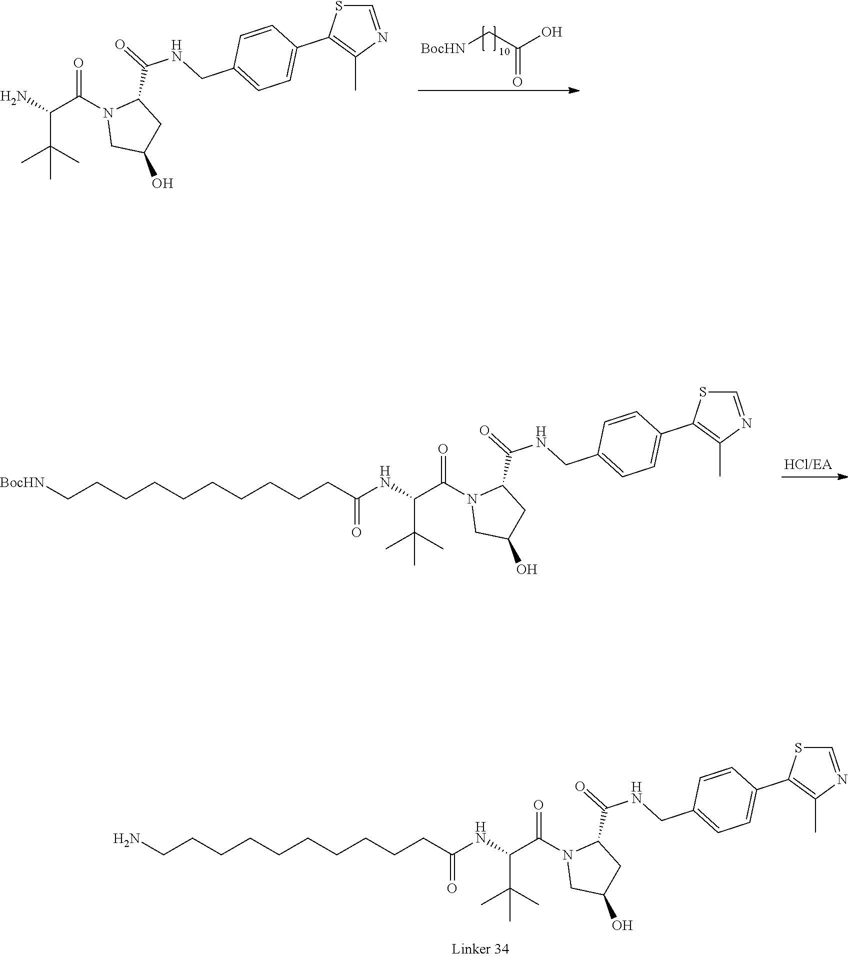

Linker 34 was synthesized following the same procedures as Linker 25 as described for Example 25. (1.10 g, yield: 44%). ¹H NMR (400 MHz, DMSO-d₆) δ 8.99 (s, 1H), 8.61 (t, J=6.4 Hz, 1H), 7.87 (d, J=8.8 Hz, 1H), 7.41 (dd, J=17.6, 8.0 Hz, 4H), 4.55 (d, J=9.6 Hz, 1H), 4.49-4.40 (m, 2H), 4.36 (brs, 1H), 4.26-4.17 (m, 1H), 3.70-3.64 (m, 2H), 2.59-2.52 (m, 2H), 2.45 (s, 3H), 2.31-2.22 (m, 1H), 2.16-2.08 (m, 1H), 2.06-1.99 (m, 1H), 1.96-1.86 (m, 1H), 1.56-1.42 (m, 2H), 1.39-1.30 (m, 2H), 1.28-1.19 (m, 12H), 0.94 (s, 9H). MS (ESI) m/z=614.4 [M+H]⁺.

### Example 35: (2S,4R)-1-((S)-2-(2-(2-Aminoethoxy)acetamido)-3,3-dimethylbutanoyl)-4-hydroxy-N-(4-(4-methylthiazol-5-yl)benzyl)pyrrolidine-2-carboxamide (Linker 35)

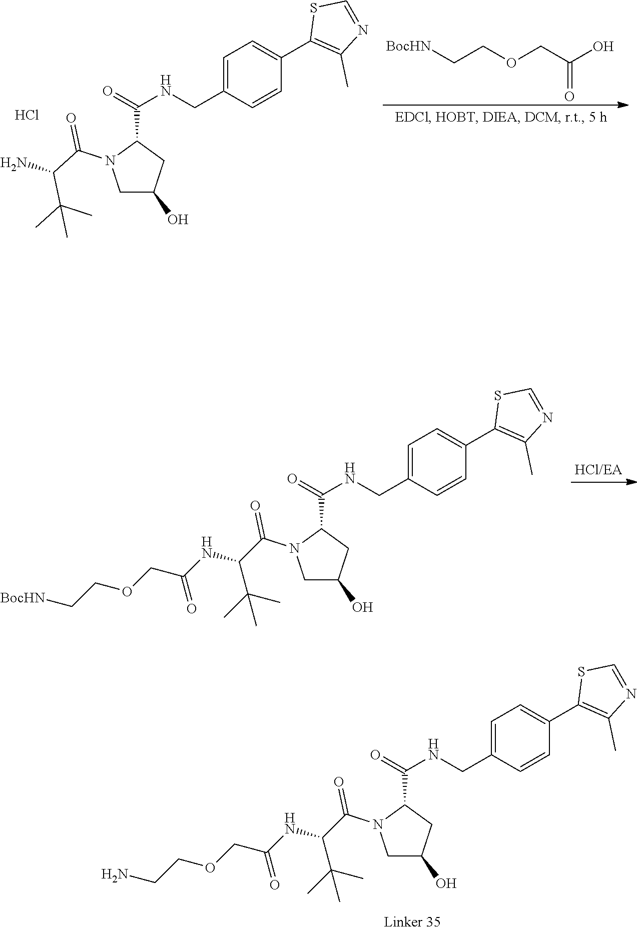 [SMILES: Cl ; Cc1ncsc1-c1ccc(CNC(=O)[C@@H]2C[C@@H](O)CN2C(=O)[C@@H](N)C(C)(C)C)cc1 ; CC(C)(C)OC(=O)NCCOCC(=O)O ; Cc1ncsc1-c1ccc(CNC(=O)[C@@H]2C[C@@H](O)CN2C(=O)[C@@H](NC(=O)COCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCN)C(C)(C)C)cc1]

Linker 35 was synthesized following the same procedures as Linker 25 as described for Example 25. (1.35 g, 2.38 mmol, yield: 79%). ¹H NMR (400 MHz, DMSO-d₆) δ 9.23 (s, 1H), 8.70 (t, J=6.0 Hz, 1H), 8.35-8.14 (m, 3H), 7.78 (d, J=9.6 Hz, 1H), 7.47-7.38 (m, 4H), 4.61 (d, J=9.6 Hz, 1H), 4.49-4.34 (m, 3H), 4.30-4.21 (m, 1H), 4.09-3.99 (m, 2H), 3.75-3.58 (m, 4H), 3.06-2.94 (m, 2H), 2.48 (s, 3H), 2.13-2.03 (m, 1H), 1.95-1.85 (m, 1H), 0.95 (s, 9H). MS (ESI) m/z=532.0 [M+H]⁺.

### Example 36: (2S,4R)-1-((S)-2-(3-(2-Aminoethoxy)propanamido)-3,3-dimethylbutanoyl)-4-hydroxy-N-(4-(4-methylthiazol-5-yl)benzyl)pyrrolidine-2-carboxamide (Linker 36)

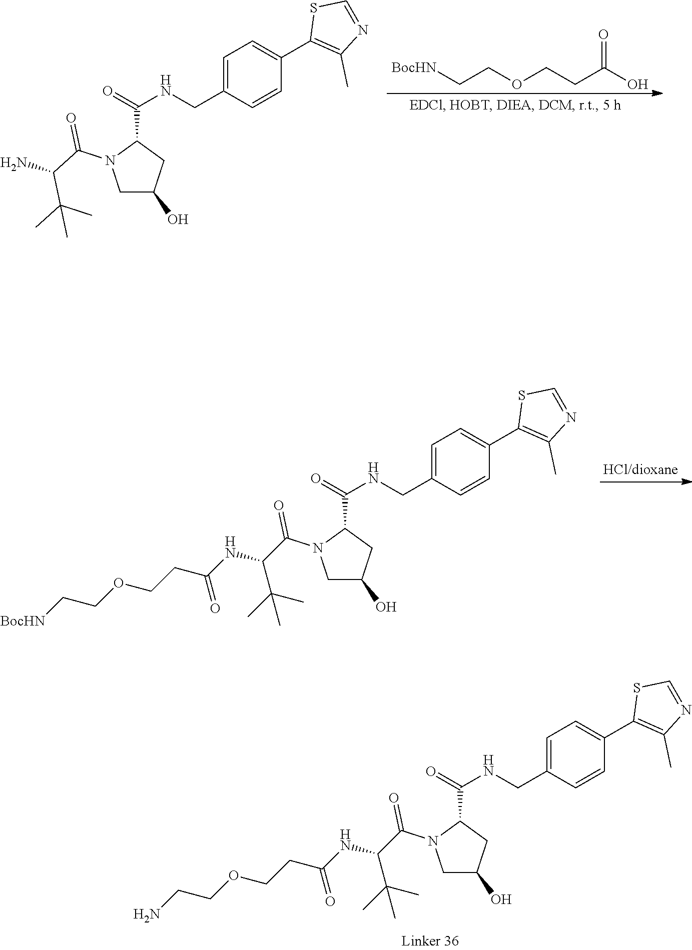 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCC(=O)O ; Cc1ncsc1-c1ccc(CNC(=O)[C@@H]2C[C@@H](O)CN2C(=O)[C@@H](NC(=O)CCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCOCCN)C(C)(C)C)cc1]

Linker 36 was synthesized following the same procedures as Linker 25 as described for Example 25. (1.32 g, 2.01 mmol, yield: 65%). ¹H NMR (400 MHz, DMSO-d₆) δ 8.99 (s, 1H), 8.57 (t, J=6.0 Hz, 1H), 8.03 (d, J=8 Hz, 1H), 7.85 (s, 3H), 7.43-7.37 (m, 4H), 4.57 (d, J=9.2 Hz, 1H), 4.46-4.31 (m, 3H), 4.26-4.20 (m, 1H), 3.69-3.55 (m, 6H), 3.99-2.95 (m, 2H), 2.60-2.56 (m, 1H), 2.46-2.42 (m, 4H), 2.05-2.03 (m, 1H), 1.93-1.92 (m, 1H), 0.95 (s, 9H). MS (ESI) m/z=546.0 [M+H]⁺.

### Example 37: (2S,4R)-1-((S)-2-(2-(2-(2-Aminoethoxy)ethoxy)acetamido)-3,3-dimethylbutanoyl)-4-hydroxy-N-(4-(4-methylthiazol-5-yl)benzyl)pyrrolidine-2-carboxamide Linker 37)

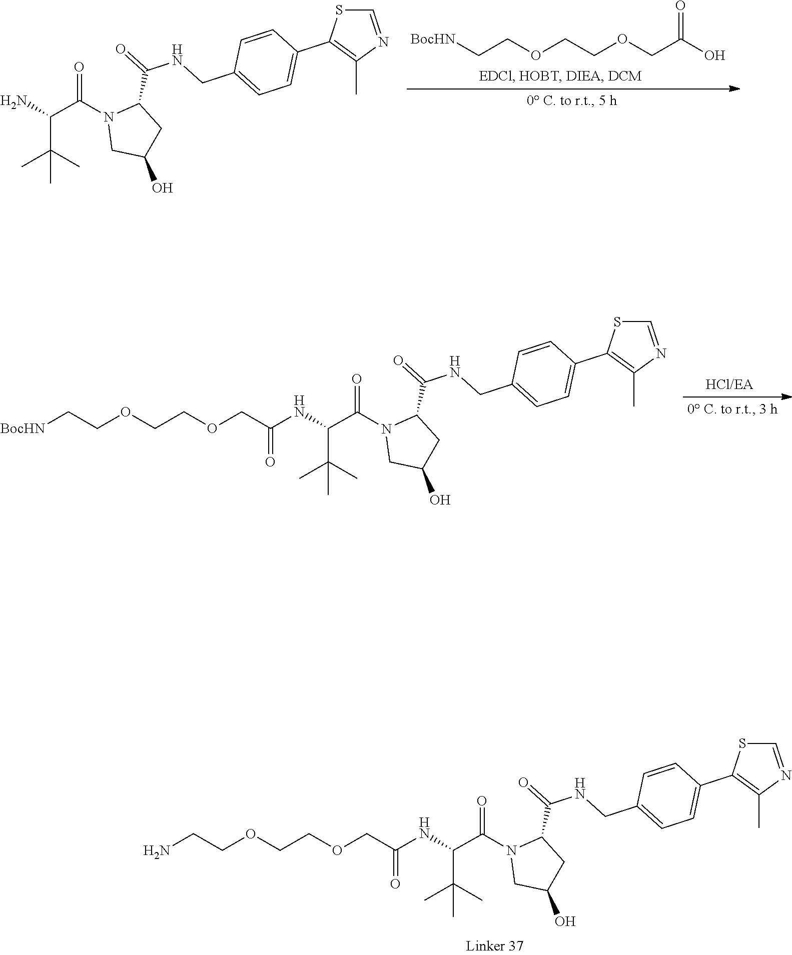 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCOCC(=O)O ; Cc1ncsc1-c1ccc(CNC(=O)[C@@H]2C[C@@H](O)CN2C(=O)[C@@H](NC(=O)COCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCOCCN)C(C)(C)C)cc1]

Linker 37 was synthesized following the same procedures as Linker 25 as described for Example 25. (1.2 g, yield: 94%). ¹H NMR (400 MHz, DMSO-d₆) δ 9.38 (s, 1H), 8.78 (t, J=6.0 Hz, 1H), 8.18 (s, 3H), 7.59-7.37 (m, 5H), 4.58 (d, J=9.6 Hz, 1H), 4.49 (t, J=8.2 Hz, 1H), 4.42-4.26 (m, 3H), 4.09-3.95 (m, 2H), 3.72-3.55 (m, 8H), 2.99-2.92 (m, 2H), 2.49 (s, 3H), 2.15-2.04 (m, 1H), 1.95-1.85 (m, 1H), 0.95 (s, 9H). MS (ESI) m/z=576.1 [M+H]⁺.

### Example 38: (2S,4R)-1-((S)-2-(3-(2-(2-Aminoethoxy)ethoxy)propanamido)-3,3-dimethylbutanoyl)-4-hydroxy-N-(4-(4-methylthiazol-5-yl)benzyl)pyrrolidine-2-carboxamide (Linker 38)

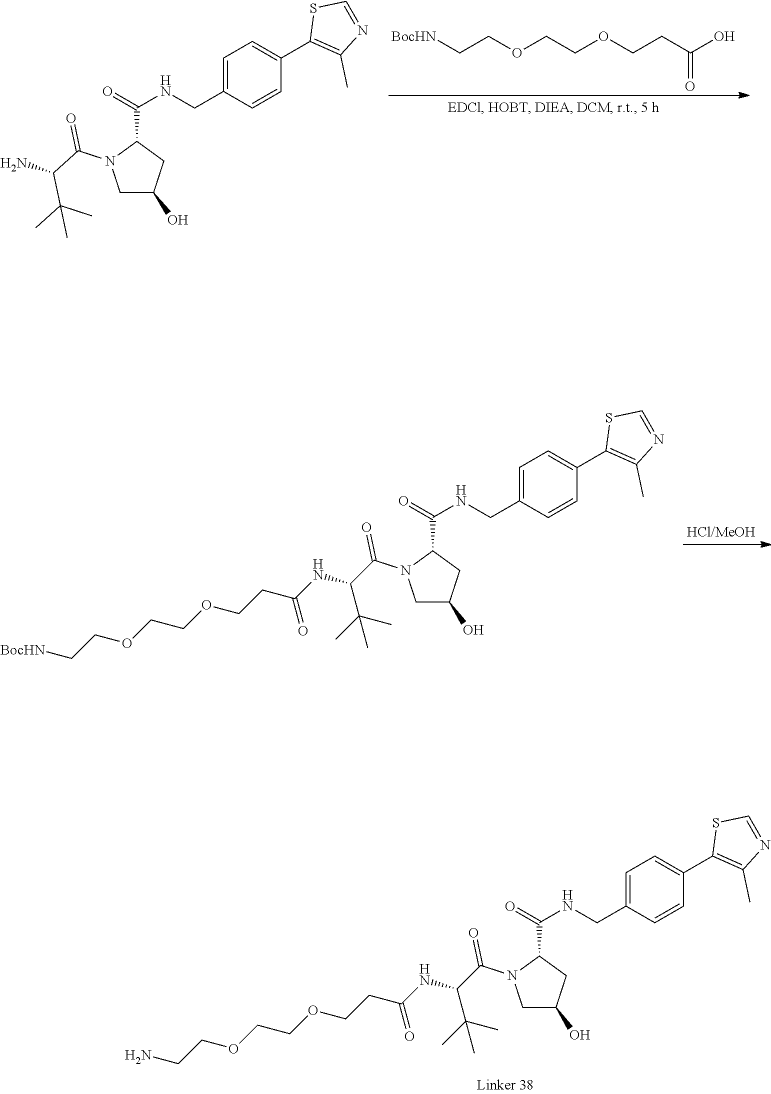 [SMILES: Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCOCCC(=O)O ; Cc1ncsc1-c1ccc(CNC(=O)[C@@H]2C[C@@H](O)CN2C(=O)[C@@H](NC(=O)CCOCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)CCOCCOCCN)C(C)(C)C)cc1]

Linker 38 was synthesized following the same procedures as Linker 25 as described for Example 25. (1.34 g, 1.94 mmol, yield: 65%). ¹H NMR (400 MHz, DMSO-d₆) δ 9.02 (s, 1H), 8.58 (t, J=6.0 Hz, 1H), 7.94 (d, J=8 Hz, 1H), 7.82 (s, 3H), 7.42-7.30 (m, 4H), 4.58 (d, J=9.2 Hz, 1H), 4.60-4.37 (m, 3H), 4.25-4.31 (m, 1H), 3.70-3.50 (m, 10H), 3.00-2.96 (m, 2H), 2.57-2.55 (m, 1H), 2.45 (s, 3H), 2.41-2.38 (m, 1H), 2.06-2.04 (m, 1H), 1.95-1.93 (m, 1H), 0.95 (s, 9H). MS (ESI) m/z=590.1 [M+H]⁺.

### Example 39: (2S,4R)-1-((S)-14-Amino-2-(tert-butyl)-4-oxo-6,9,12-trioxa-3-azatetradecanoyl)-4-hydroxy-N-(4-(4-methylthiazol-5-yl)benzyl)pyrrolidine-2-carboxamide (Linker 39)

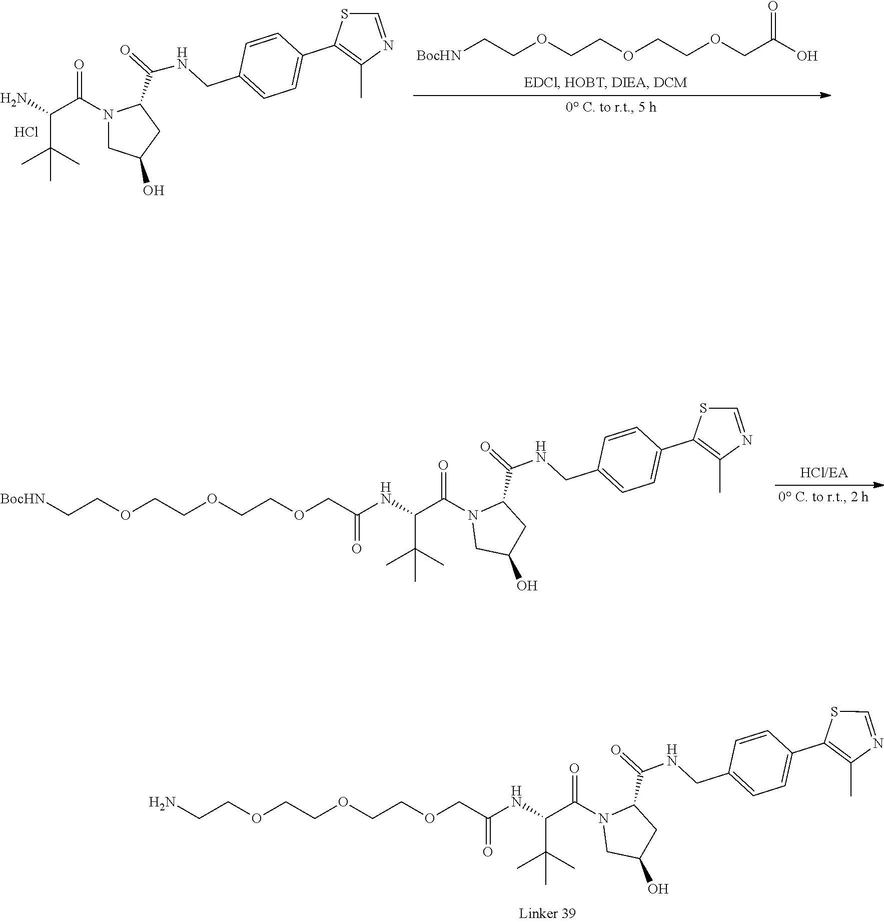 [SMILES: Cl ; Cc1ncsc1-c1ccc(CNC(=O)[C@@H]2C[C@@H](O)CN2C(=O)[C@@H](N)C(C)(C)C)cc1 ; CC(C)(C)OC(=O)NCCOCCOCCOCC(=O)O ; Cc1ncsc1-c1ccc(CNC(=O)[C@@H]2C[C@@H](O)CN2C(=O)[C@@H](NC(=O)COCCOCCOCCNC(=O)OC(C)(C)C)C(C)(C)C)cc1 ; Cc1ncsc1-c1ccc(CNC(=O)[C@@H]2C[C@@H](O)CN2C(=O)[C@@H](NC(=O)COCCOCCOCCN)C(C)(C)C)cc1]

Linker 39 was synthesized following the same procedures as Linker 25 as described for Example 25. (1.53 g, yield: 77%). ¹H NMR (400 MHz, DMSO-d₆) δ 9.01 (s, 1H), 8.59 (t, J=6.0 Hz, 1H), 7.81 (s, 3H), 7.48-7.41 (m, 5H), 4.58 (d, J=9.6 Hz, 1H), 4.47-4.26 (m, 4H), 3.99 (s, 2H), 3.70-3.58 (m, 12H), 3.0-2.96 (m, 2H), 2.46 (s, 3H), 2.11-2.06 (m, 1H), 1.95-1.88 (m, 1H), 0.96 (s, 9H). MS (ESI) m/z=621.1 [M+H]⁺.

### Example 40: (2S,4R)-1-((S)-1-Amino-14-(tert-butyl)-12-oxo-3,6,9-trioxa-13-azapentadecan-15-oyl)-4-hydroxy-N-(4-(4-methylthiazol-5-yl)benzyl)pyrrolidine-2-carboxamide (Linker 40)

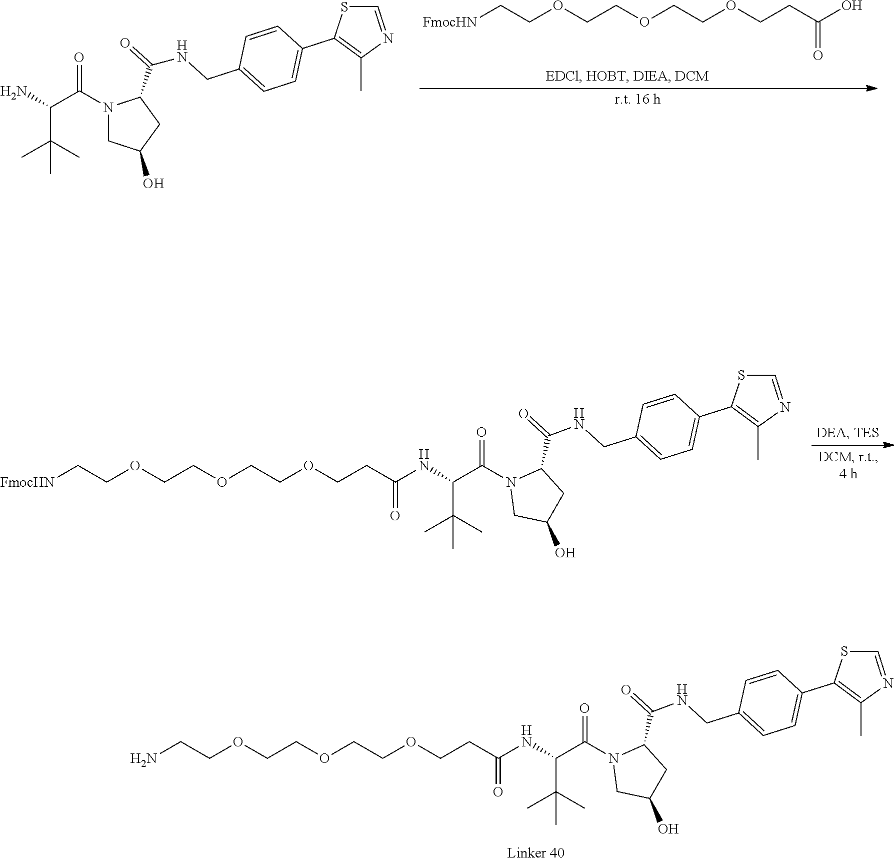

Linker 40 was synthesized following the same procedures as Linker 25 as described for Example 25. (1.52 g, yield: 64%). ¹H NMR (400 MHz, DMSO-d₆) δ 9.01 (s, 1H), 8.57 (t, J=6.0 Hz, 1H), 7.91 (d, J=9.2 Hz, 1H), 7.81 (s, 3H), 7.44-7.38 (m, 4H), 4.58-4.55 (m, 1H), 4.45-4.36 (m, 3H), 4.25-4.21 (m, 1H), 3.70-3.48 (m, 14H), 3.00-2.97 (m, 2H), 2.59-2.52 (m, 1H), 2.46 (s, 3H), 2.39-2.34 (m, 1H), 2.08-2.03 (m, 1H), 1.95-1.88 (m, 1H), 0.94 (s, 9H). MS (ESI) m/z=633.8 [M+H]⁺.

### Example 41: (2S,4R)-1-((S)-1-Amino-17-(tert-butyl)-15-oxo-3,6,9,12-tetraoxa-16-azaoctadecan-18-oyl)-4-hydroxy-N-(4-(4-methylthiazol-5-yl)benzyl)pyrrolidine-2-carboxamide (Linker 41)

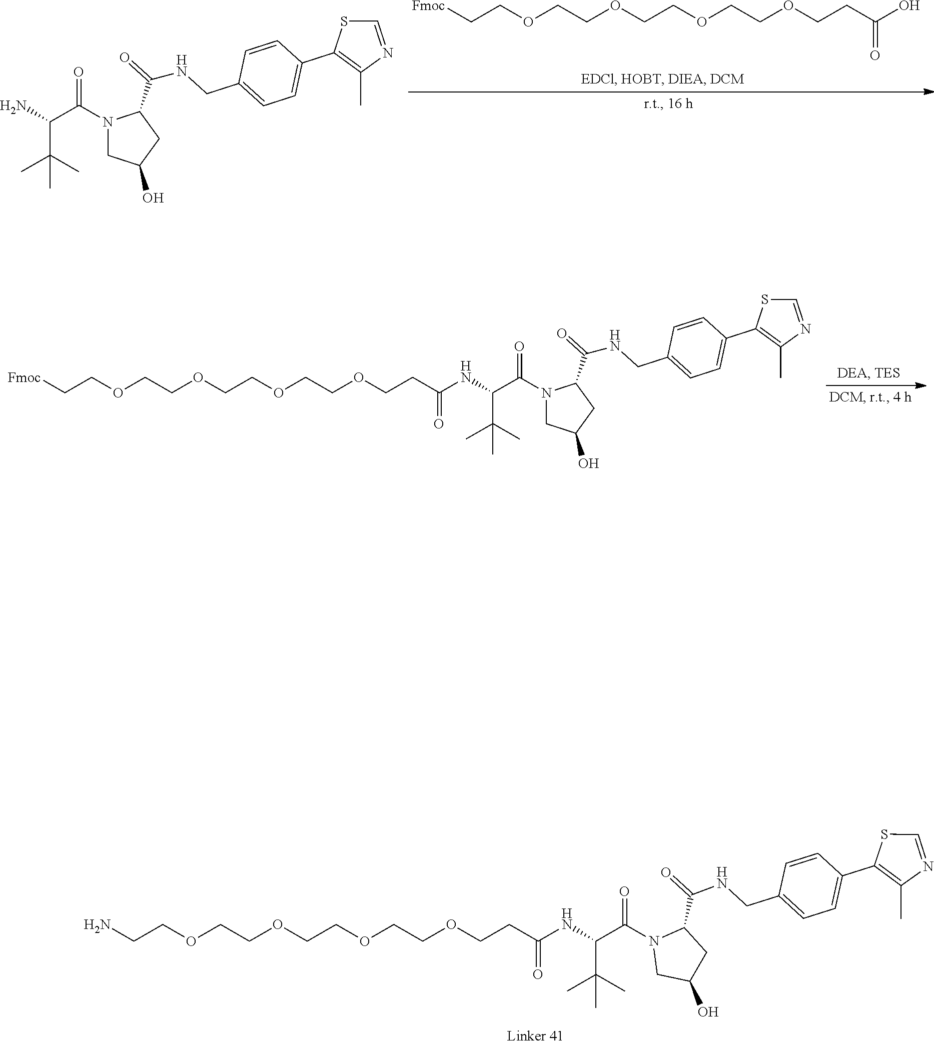

Linker 41 was synthesized following the same procedures as Linker 25 as described for Example 25. (1.12 g, yield: 52%). ¹H NMR (400 MHz, DMSO-d₆) δ 8.98 (s, 1H), 8.58 (t, J=5.6 Hz, 1H), 7.92 (d, J=9.2 Hz, 1H), 7.44-7.38 (m, 4H), 4.56 (d, J=9.2 Hz, 1H), 4.47-4.41 (m, 2H), 4.38-4.34 (m, 1H), 4.26-4.19 (m, 1H), 3.70-3.55 (m, 5H), 3.53-3.45 (m, 14H), 3.35 (t, J=5.6 Hz, 2H), 2.64 (t, J=5.6 Hz, 2H), 2.58-2.50 (m, 1H), 2.45 (s, 3H), 2.40-2.35 (m, 1H), 2.08-2.00 (m, 1H), 1.94-1.91 (m, 1H), 0.94 (s, 9H). MS (ESI) m/z=678.1 [M+H]⁺.

### Example 42: (2S,4R)-1-((S)-1-Amino-20-(tert-butyl)-18-oxo-3,6,9,12,15-pentaoxa-19-azahenicosan-21-oyl)-4-hydroxy-N-(4-(4-methylthiazol-5-yl)benzyl)pyrrolidine-2-carboxamide (Linker 42)

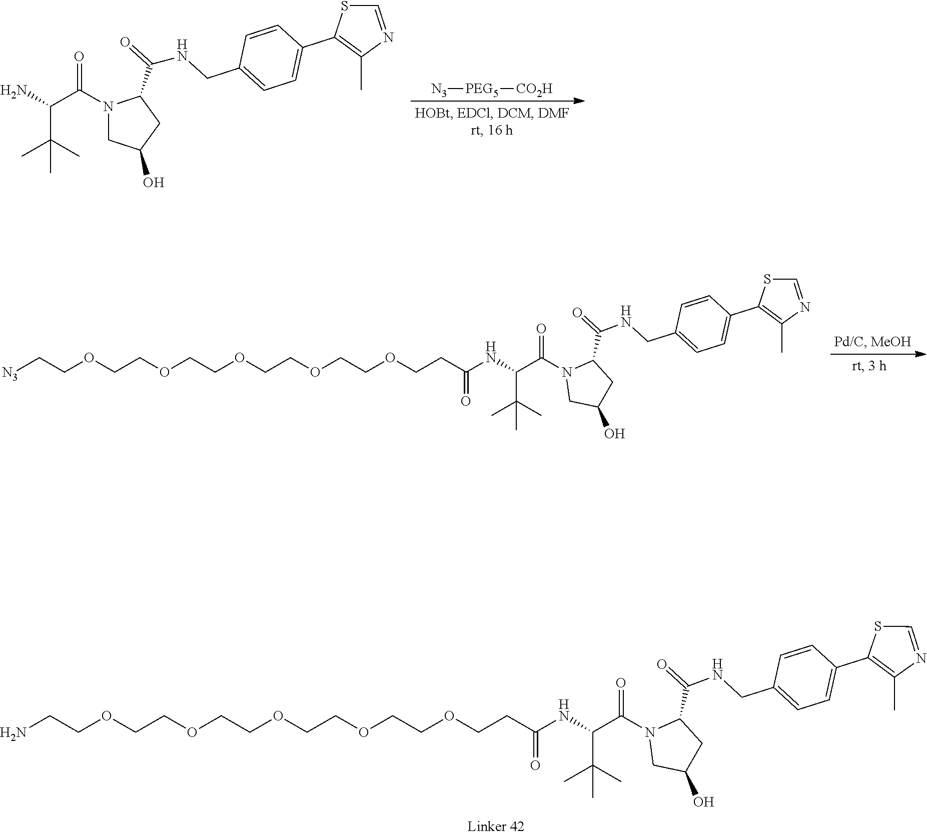

Linker 42 was synthesized following the same procedures as Linker 25 as described for Example 25. (1.1 g, yield: 42%). ¹H NMR (400 MHz, DMSO-d₆) δ 9.38 (s, 1H), 8.67 (t, J=16 Hz, 1H), 8.14 (br, 3H), 7.91 (d, J=9.2 Hz, 1H), 7.39-7.48 (m, 4H), 4.53 (d, J=9.2 Hz, 1H), 4.39-4.46 (m, 2H), 4.36-4.34 (m, 1H), 4.20-4.25 (m, 1H), 3.45-3.68 (m, 22H), 2.91-2.95 (m, 2H), 2.52-2.58 (m, 1H), 2.47 (s, 3H), 2.32-2.39 (m, 1H), 2.03-2.08 (m, 1H), 1.85-1.92 (m, 1H), 0.92 (s, 9H). MS (ESI) m/z=722.4 [M+H]⁺.

### Example 43: 4-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid (Linker 43)

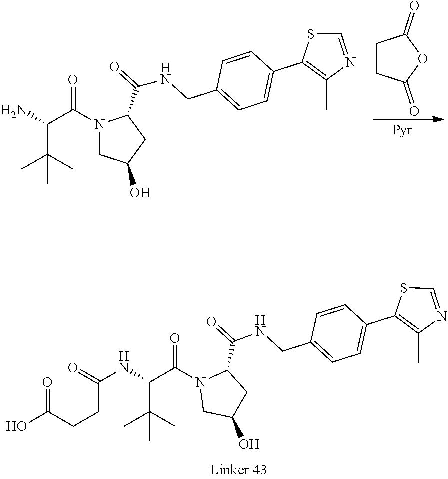

A mixture of (2S,4R)-1-((S)-2-amino-3,3-dimethylbutanoyl)-4-hydroxy-N-(4-(4-methylthiazol-5-yl)benzyl)pyrrolidine-2-carboxamide (1.0 g, 2.3 mmol) and succinic anhydride (465 mg, 4.65 mmol) in pyridine (5 mL) was stirred at rt for overnight. The mixture was concentrated. The resulting residue was purified by flash chromatography (reversed-phase, MeCN/H₂O) to give the title compound Linker 43 (1.05 g, yield: 86%). ¹H NMR (400 MHz, DMSO-d₆): δ12.02 (s, 1H), 8.99 (s, 1H), 8.58 (t, J=6.0 Hz, 1H), 7.96 (d, J=9.2 Hz, 1H), 7.43-7.37 (m, 4H), 5.13 (d, J=3.6 Hz, 1H), 4.53 (d, J=9.2 Hz, 1H), 4.46-4.40 (m, 2H), 4.34 (s, 1H), 4.21 (dd, J=16.0, 5.2 Hz, 1H), 3.69-3.60 (m, 2H), 2.45 (s, 3H), 2.44-2.33 (m, 4H), 2.06-2.01 (m, 1H), 1.93-1.87 (m, 1H), 0.93 (s, 9H). ¹³C NMR (100 MHz, DMSO-d6): δ173.83, 171.92, 170.86, 169.56, 151.41, 147.70, 139.48, 131.15, 129.63, 128.62, 127.41, 68.87, 58.70, 56.44, 56.34, 41.65, 37.91, 35.35, 29.74, 29.25, 26.35, 15.92. MS (ESI) m/z=531.2 [M+H]⁺.

### Example 44: 5-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (Linker 44)

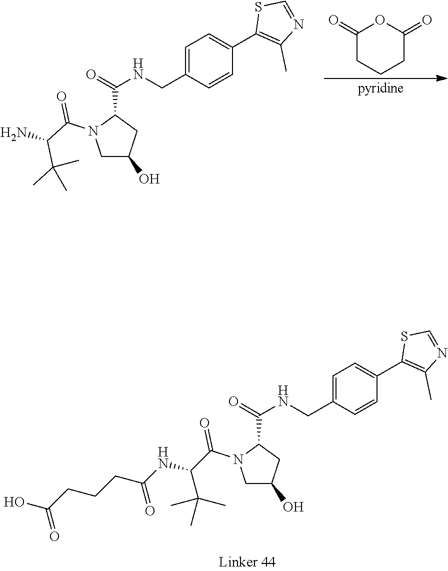

Linker 44 was synthesized following the same procedures as Linker 43 as described for Example 43. (1.5 g, yield: 79%). ¹H NMR (400 MHz, DMSO-d₆): δ8.99 (s, 1H), 8.59 (t, J=6.0 Hz, 1H), 7.91 (d, J=9.2 Hz, 1H), 7.44-7.37 (m, 4H), 5.16 (brs, 1H), 4.54 (d, J=9.2 Hz, 1H), 4.47-4.42 (m, 2H), 4.36 (s, 1H), 4.21 (dd, J=16.0, 5.2 Hz, 1H), 3.7-3.64 (m, 2H), 2.45 (s, 3H), 2.31-2.14 (m, 4H), 2.07-2.02 (m, 1H), 1.94-1.81 (m, 1H), 1.74-1.68 (m, 2H), 0.94 (s, 9H).

¹³C NMR (100 MHz, DMSO-d₆): δ 174.18, 171.94, 171.63, 169.66, 151.41, 147.70, 139.46, 131.15, 129.61, 128.62, 127.41, 68.86, 58.69, 56.38, 41.65, 37.91, 35.16, 34.03, 33.10, 26.35, 20.89, 15.92. MS (ESI) m/z=543.2 [M+H]⁺.

### Example 45: 6-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (Linker 45)

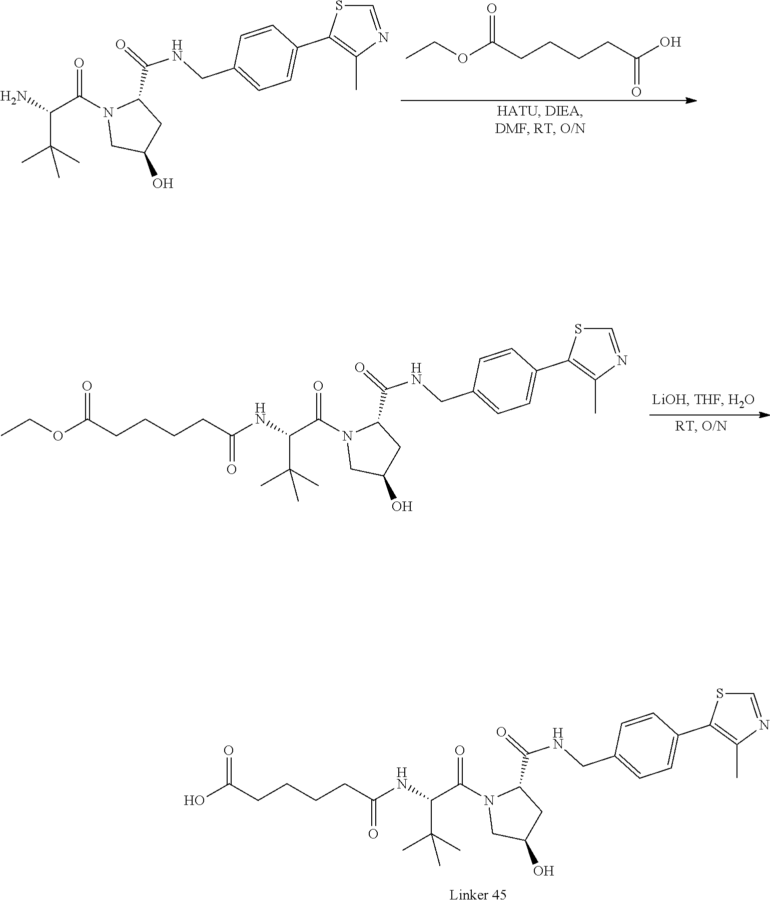

Linker 45 was synthesized following the same procedures as Linker 25 as described for Example 25. (1.198 g, yield: 74%). ¹H NMR (400 MHz, CDCl₃) δ 8.68 (s, 1H), 7.75 (s, 1H), 7.32-7.27 (m, 5H), 4.64-4.57 (m, 3H), 4.56-4.50 (m, 1H), 4.28-4.25 (m, 1H), 4.02-3.99 (m, 1H), 3.71-3.68 (m, 1H), 2.47 (s, 3H), 2.24-2.18 (m, 6H), 1.59-1.48 (m, 4H), 0.96 (s, 9H). MS (ESI) m/z=559.3 [M+H]⁺.

### Example 46: 7-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-7-oxoheptanoic acid (Linker 46)

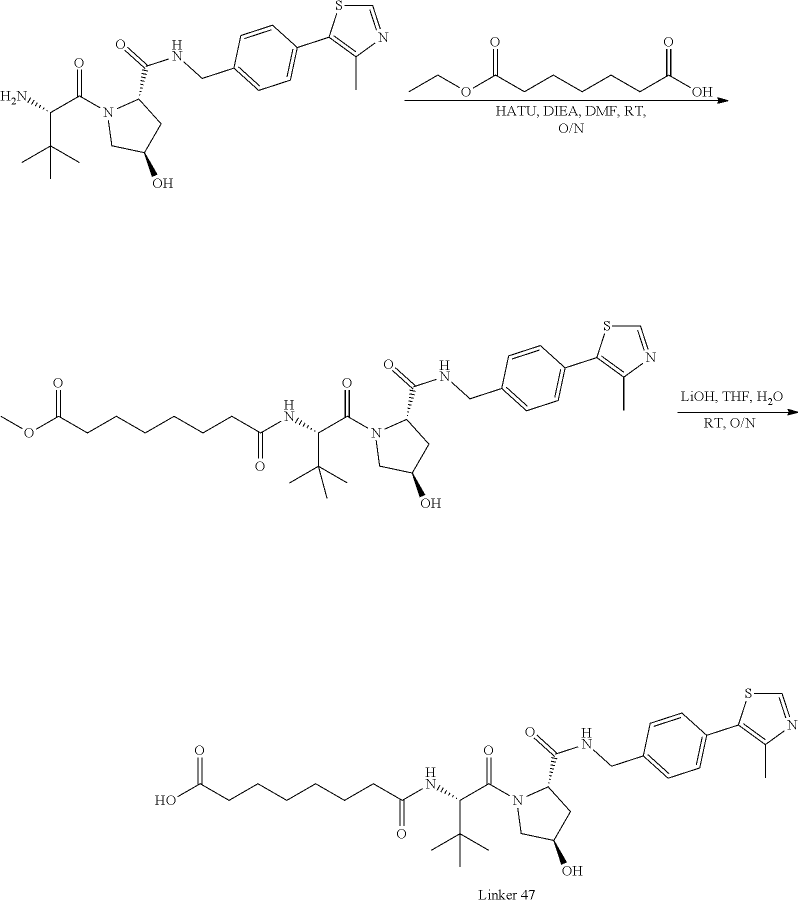 [SMILES: Cc1ncsc1-c1ccc(CNC(=O)[C@@H]2C[C@@H](O)CN2C(=O)[C@@H](N)C(C)(C)C)cc1 ; CCOC(=O)CCCCCC(=O)O ; COC(=O)CCCCCCC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)CCCCCCC(=O)O)C(C)(C)C)cc1]

Linker 46 was synthesized following the same procedures as Linker 45 as described for Example 45. (1.099 g, yield: 50%). ¹H NMR (400 MHz, CDCl₃) δ 8.67 (s, 1H), 7.56-7.55 (m, 1H), 7.34-7.30 (m, 5H), 4.68-4.59 (m, 3H), 4.59-4.51 (m, 1H), 4.25 (dd, J=4.8 Hz, 15.2 Hz, 1H), 4.06-4.03 (m, 1H), 3.70-3.68 (m, 1H), 2.46 (s, 3H), 2.31-2.11 (m, 6H), 1.55-1.51 (m, 4H), 1.29-1.24 (m, 2H), 0.94 (s, 9H). MS (ESI) m/z=573.1 [M+H]⁺.

### Example 47: 8-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (Linker 47)

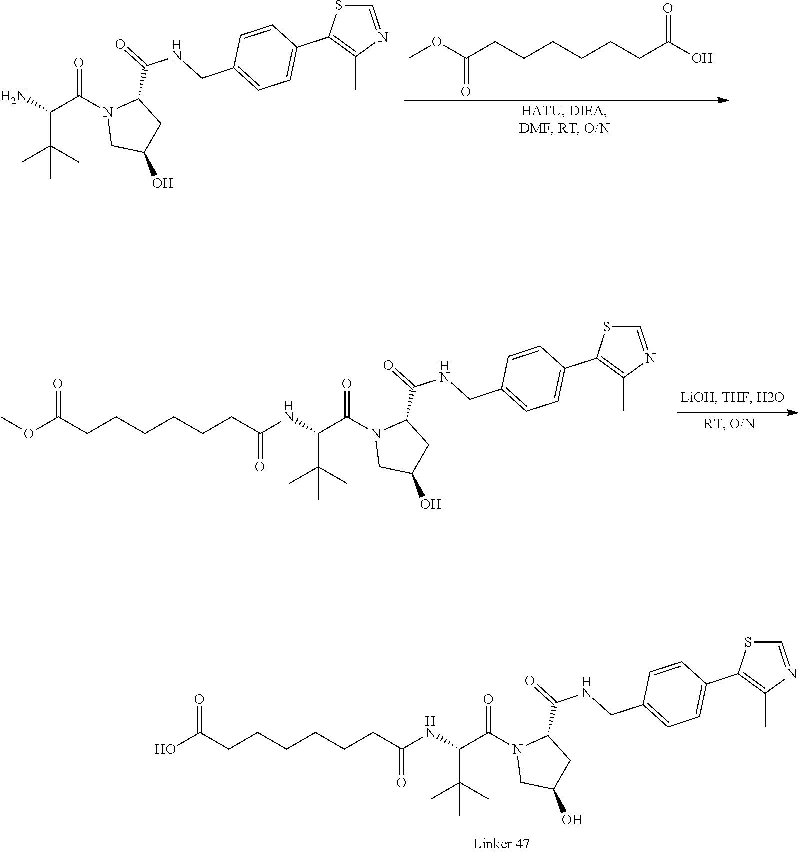

Linker 47 was synthesized following the same procedures as Linker 45 as described for Example 45. (1.08 g, yield: 60%). ¹H NMR (400 MHz, DMSO-d₆) δ 8.99 (s, 1H), 8.55 (t, J=2.4 Hz, 1H), 7.83 (d, J=9.2 Hz, 1H), 7.44-7.38 (m, 4H), 4.55 (d, J=9.6 Hz, 1H), 4.52-4.41 (m, 2H), 4.36 (s, 1H), 4.25-4.21 (m, 1H), 3.67-3.66 (m, 2H), 2.45 (s, 3H), 2.30-1.91 (m, 6H), 1.49-1.47 (m, 4H), 1.26-1.24 (m, 4H), 0.92 (s, 9H). MS (ESI) m/z=587.3 [M+H]⁺.

### Example 48: 9-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-9-oxononanoic acid (Linker 48)

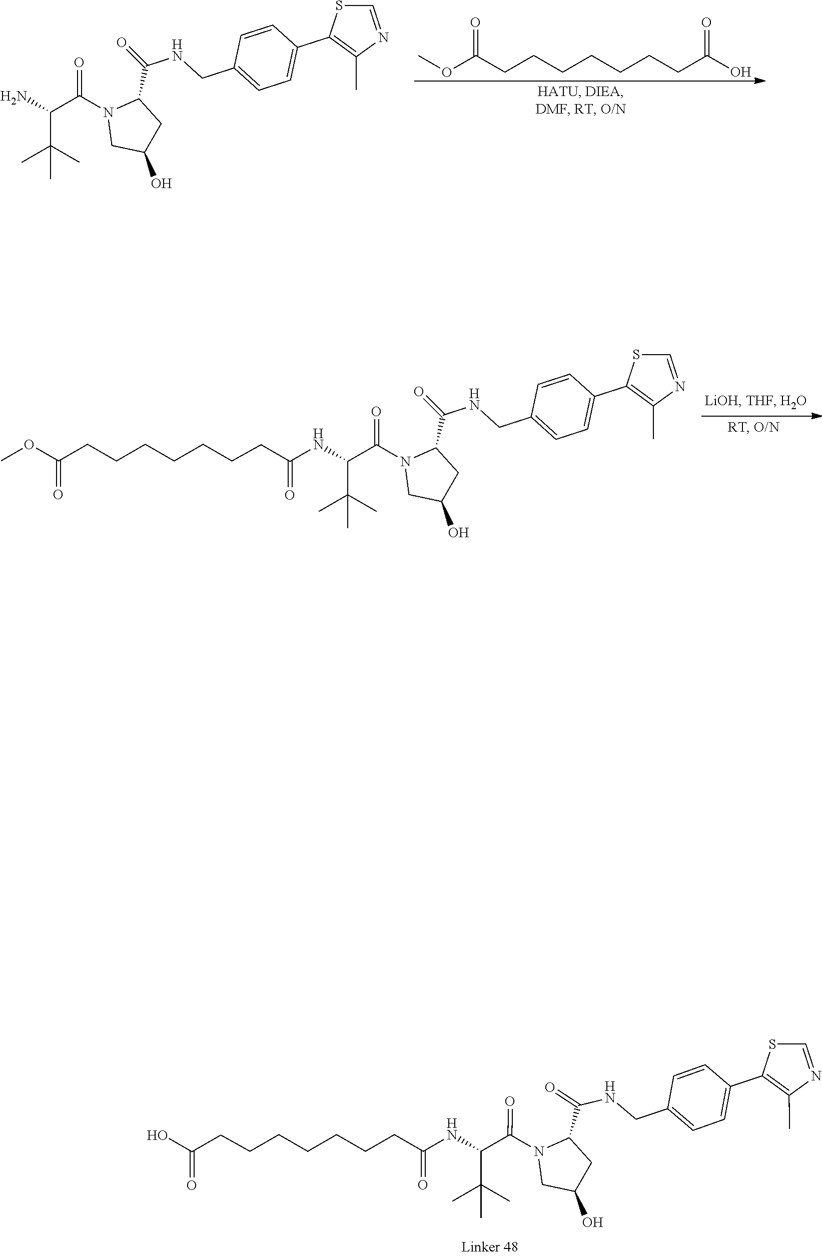

Linker 48 was synthesized following the same procedures as Linker 45 as described for Example 45. (1.155 g, yield: 56%). ¹H NMR (400 MHz, CDCl₃) δ 8.70 (s, 1H), 7.55 (s, 1H), 7.33-7.27 (i, 4H), 7.08 (d, J=8.0 Hz, 1H), 4.68-4.52 (i, 4H), 4.31-4.27 (i, 1H), 4.08-4.05 (m, 1H), 3.69-3.67 (m, 1H), 2.48 (s, 3H), 2.33-2.11 (m, 6H), 1.60-1.47 (b, 4H), 1.29-1.20 (m, 6H), 0.96 (s, 9H). MS (ESI) m/z=601.1 [M+H]⁺.

### Example 49: 10-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-11-oxodecanoic acid (Linker 49)

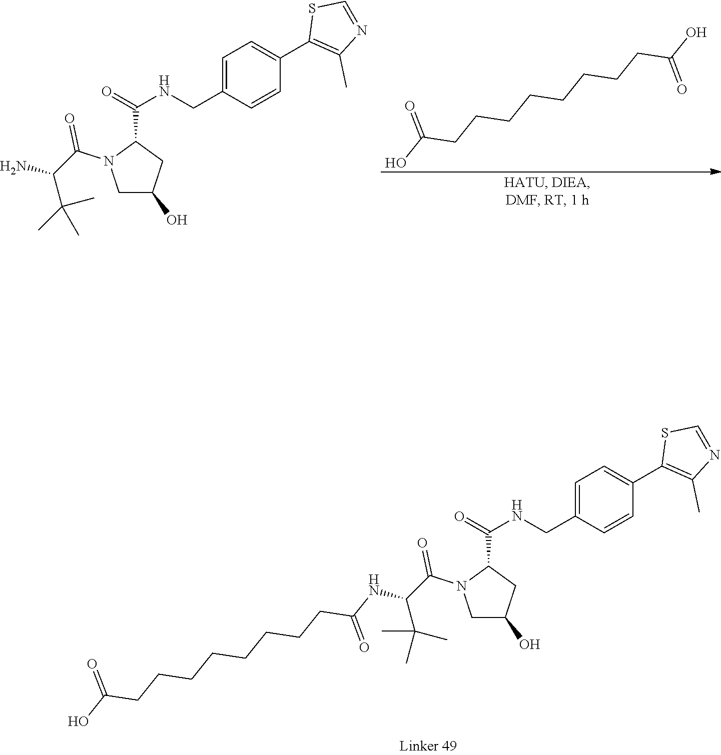 [SMILES: Cc1ncsc1-c1ccc(CNC(=O)[C@@H]2C[C@@H](O)CN2C(=O)[C@@H](N)C(C)(C)C)cc1 ; O=C(O)CCCCCCCCC(=O)O ; Cc1ncsc1-c1ccc(CNC(=O)[C@@H]2C[C@@H](O)CN2C(=O)[C@@H](NC(=O)CCCCCCCCC(=O)O)C(C)(C)C)cc1]

Linker 49 was synthesized following the same procedure as Linker 45 as described for Example 45. (1.1 g, yield: 35.5%). ¹H NMR (400 MHz, DMSO-d₆) 58.99 (s, 1H), 8.58 (t, J=6.0 Hz, 1H), 7.85 (d, J=9.2 Hz, 1H), 7.43-7.37 (m, 4H), 4.54 (d, J=9.2 Hz, 1H), 4.47-4.41 (m, 2H), 4.35 (s, 1H), 4.21 (dd, J=16.0, 5.6 Hz, 1H), 3.69-3.63 (m, 2H), 2.45 (s, 3H), 2.29-2.09 (m, 4H), 2.03-2.01 (m, 1H), 1.94-1.88 (m, 1H), 1.47 (m, 4H), 1.24 (b, 8H), 0.94 (s, 9H).

¹³C NMR (100 MHz, DMSO-d₆): δ 172.07, 171.92, 169.69, 151.41, 147.70, 139.48, 131.14, 129.62, 128.61, 127.40, 68.84, 58.67, 56.32, 56.26, 41.64, 37.93, 35.18, 34.85, 28.62, 26.36, 25.39, 15.93. MS (ESI) m/z=615.3 [M+H]⁺.

### Example 50: 11-(((S)-1-((2S,4R)-4-Hydroxy-2-((4-(4-methylthiazol-5-yl)benzyl)carbamoyl)pyrrolidin-1I-yl)-3,3-dimethyl-1I-oxobutan-2-yl)amino)-11-oxoundecanoic acid (Linker 50)

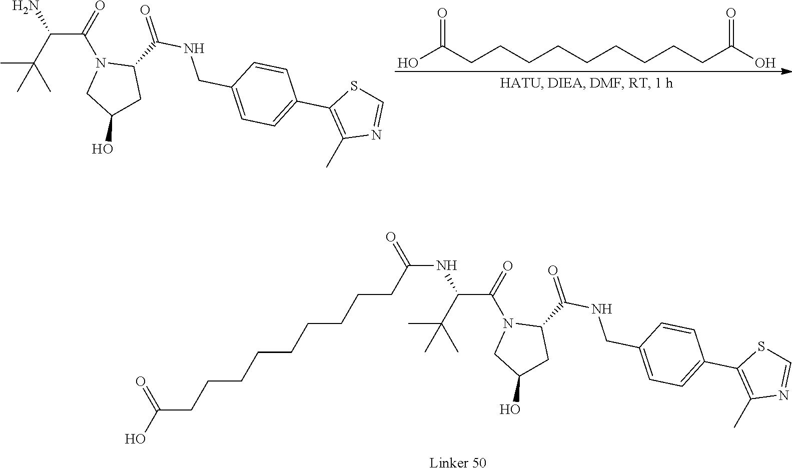 [SMILES: Cc1ncsc1-c1ccc(CNC(=O)[C@@H]2C[C@@H](O)CN2C(=O)[C@@H](N)C(C)(C)C)cc1 ; O=C(O)CCCCCCCCCC(=O)O ; Cc1ncsc1-c1ccc(CNC(=O)[C@@H]2C[C@@H](O)CN2C(=O)[C@@H](NC(=O)CCCCCCCCCC(=O)O)C(C)(C)C)cc1]

Linker 50 was synthesized following the same procedure as Linker 45 as described for Example 45. (1.1 g, yield: 50%). ¹H NMR (400 MHz, DMSO-d₆) δ8.99 (s, 1H), 8.58 (t, J=6.0 Hz, 1H), 7.85 (t, J=9.2 Hz, 1H), 7.37-7.43 (m, 4H), 4.56-4.19 (m, 5H), 3.70-3.60 (m, 2H), 2.45 (s, 3H), 2.27-1.90 (m, 6H), 1.49-1.45 (m, 4H), 1.23 (m, 10H), 0.93 (s, 9H).

¹³C NMR (100 MHz, DMSO-d₆): δ174.59, 172.07, 171.92, 169.69, 151.42, 147.70, 139.49, 131.14, 129.62, 128.61, 127.41, 68.84, 58.67, 56.32, 56.25, 41.64, 37.93, 35.19, 34.85, 33.80, 28.82, 28.70, 28.68, 28.62, 28.55, 26.37, 25.42, 24.55, 15.93. MS (ESI) m/z=629.4 [M+H]⁺.

### Example 51: 3-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)propanoic acid (Linker 51)

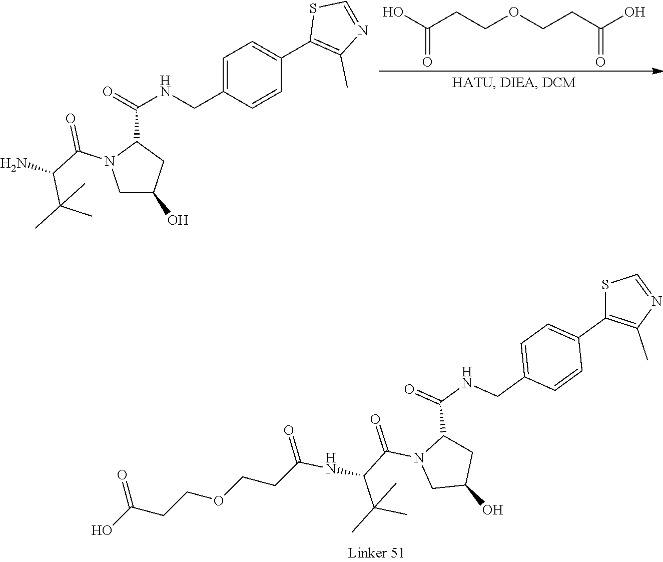

Linker 51 was synthesized following the same procedure as Linker 45 as described for Example 45. (1.1 g, yield: 42%). ¹H NMR (400 MHz, DMSO-d₆) δ8.98 (s, 1H), 8.55 (t, J=6.0 Hz, 1H), 7.91 (d, J=9.2 Hz, 1H), 7.43-7.37 (m, 4H), 4.55-4.53 (m, 1H), 4.45-4.40 (m, 2H), 4.35 (s, 1H), 4.24-4.19 (m, 1H), 3.68-3.52 (m, 6H), 2.54-2.56 (m, 1H), 2.45-2.37 (m, 5H), 2.34-2.30 (m, 1H), 2.05-2.00 (m, 1H), 1.93-1.86 (m, 1H), 0.93 (s, 9H). MS (ESI) m/z=575 [M+H]⁺.

### Example 52: 2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetic acid (Linker 52)

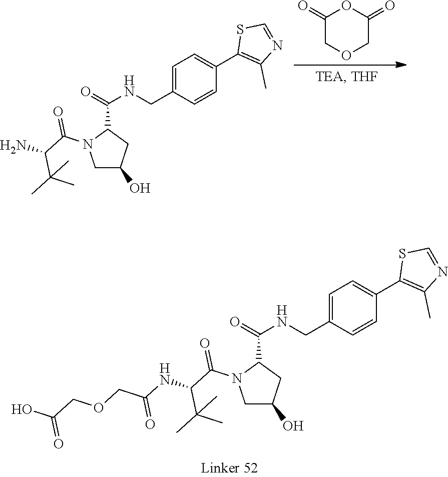

Linker 52 was synthesized following the same procedure as Linker 43 as described for Example 43. (1.2 g, yield: 63%). ¹H NMR (400 MHz, DMSO-d₆) δ12.81 (br s, 1H), 8.98 (s, 1H), 8.58 (t, J=6.0 Hz, 1H), 7.60 (d, J=9.6 Hz, 1H), 7.45-7.35 (m, 4H), 5.14 (br, 1H), 4.58-4.55 (m, 1H), 4.46-4.36 (m, 3H), 4.28-4.26 (m, 1H), 4.14 (s, 2H), 4.04 (s, 2H), 3.69-3.60 (m, 2H), 2.44 (s, 3H), 2.08-2.03 (m, 1H), 1.93-1.87 (m, 1H), 0.95 (s, 9H). MS (ESI) m/z=547 [M+H]⁺.

### Example 53: 3-(2-(3-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (Linker 53)

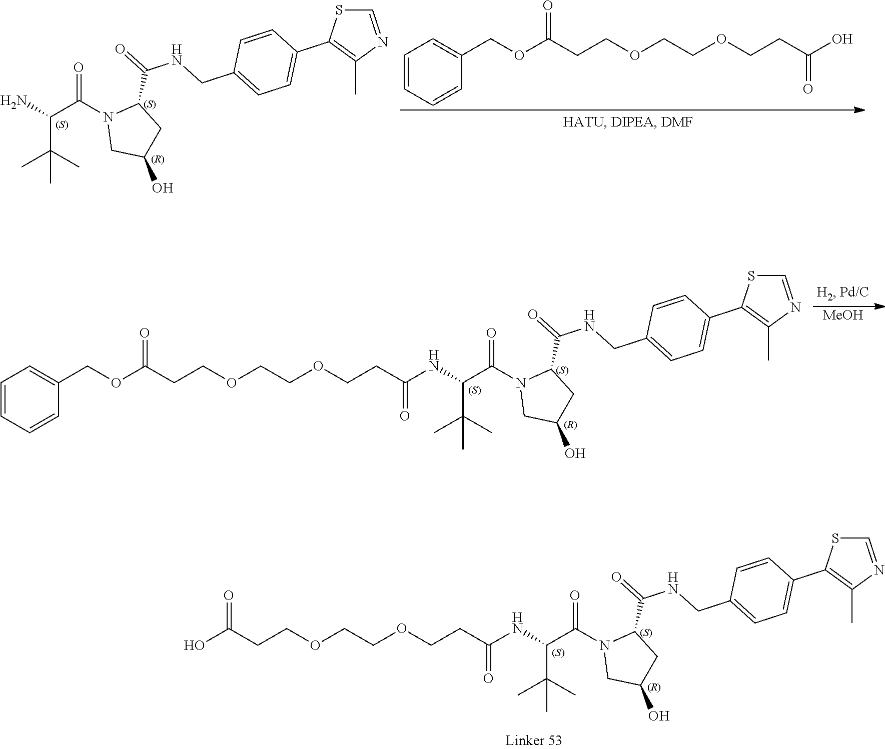

Linker 53 was synthesized following the same procedures as Linker 45 as described for Example 45. (1.4 g, yield 41%). ¹H NMR (400 MHz, DMSO-d₆): δ8.98 (s, 1H), 8.56 (t, J=6.0 Hz, 1H), 7.91 (d, J=9.2 Hz, 1H), 7.43-7.37 (m, 4H), 4.55 (d, J=9.6 Hz, 1H), 4.46-4.41 (m, 2H), 4.35 (s, 1H), 4.29-4.20 (m, 1H), 3.70-3.57 (m, 7H), 3.50-3.45 (m, 5H), 2.57-2.55 (m, 1H), 2.45 (s, 3H), 2.43-2.41 (m, 1H), 2.37-2.32 (m, 1H), 2.09-2.01 (m, 1H), 1.94-1.87 (m, 1H), 0.94 (s, 9H). MS (ESI) m/z=619.3 [M+H]⁺.

### Example 54: 2-(2-(2-(((S)-1-((2S,4R)-4-Hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetic acid (Linker 54)

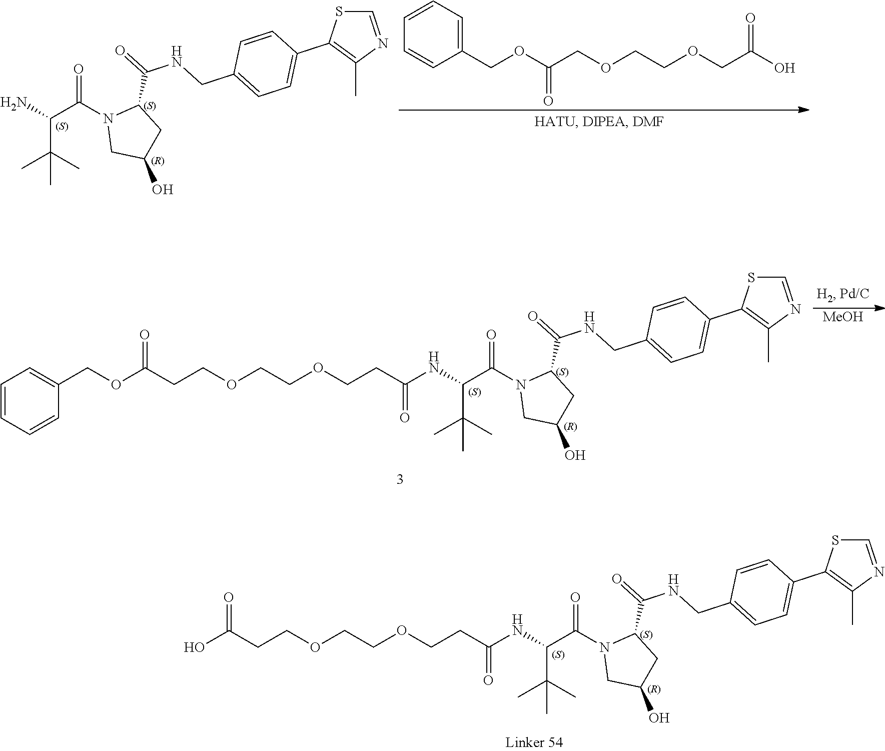

Linker 54 was synthesized following the same procedures as Linker 53 as described for Example 53. (1.126 g, yield 30%). ¹H NMR (400 MHz, DMSO-d₆) 58.98 (s, 1H), 8.60 (t, J=6.0 Hz, 1H), 7.49 (d, J=9.2 Hz, 1H), 7.40 (s, 4H), 4.57 (d, J=9.2 Hz, 1H), 4.47-4.36 (m, 3H), 4.28-4.23 (m, 1H), 4.05-3.93 (m, 4H), 3.69-3.61 (m, 6H), 2.45 (s, 3H), 2.08-2.03 (m, 1H), 1.94-1.87 (m, 1H), 0.94 (s, 9H). MS (ESI) m/z=591.2 [M+H]⁺.

### Example 55: (S)-15-((2S,4R)-4-Hydroxy-2-((4-(4-methylthiazol-5-yl)benzyl)carbamoyl)pyrrolidine-1-carbonyl)-16,16-dimethyl-13-oxo-4,7,10-trioxa-14-azaheptadecanoic acid (Linker 55)

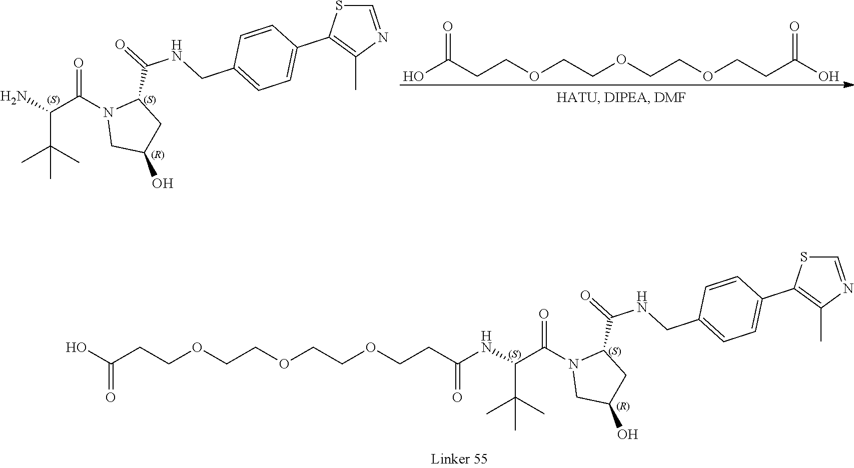

Linker 55 was synthesized following the same procedure as Linker 45 as described for Example 45. (1.7 g, yield 37%). ¹H NMR (400 MHz, DMSO-d₆) δ8.99 (s, 1H), 8.56 (t, J=6.0 Hz, 1H), 7.91 (d, J=9.6 Hz, 1H), 7.44-7.38 (m, 4H), 4.56 (d, J=9.2 Hz, 1H), 4.47-4.42 (m, 2H), 4.36 (s, 1H), 4.25-4.20 (m, 1H), 3.70-3.55 (m, 6H), 3.50-3.46 (m, 8H), 2.58-2.51 (m, 3H), 2.45-2.42 (m, 5H), 2.40-2.33 (m, 1H), 2.07-2.02 (m, 1H), 1.94-1.88 (m, 1H), 0.94 (s, 9H). LCMS (ESI) m/z=661.0 [M−H]⁻.

### Example 56: (S)-13-((2S,4R)-4-Hydroxy-2-((4-(4-methylthiazol-5-yl)benzyl)carbamoyl)pyrrolidine-1-carbonyl)-14,14-dimethyl-11-oxo-3,6,9-trioxa-12-azapentadecanoic acid (Linker 56)

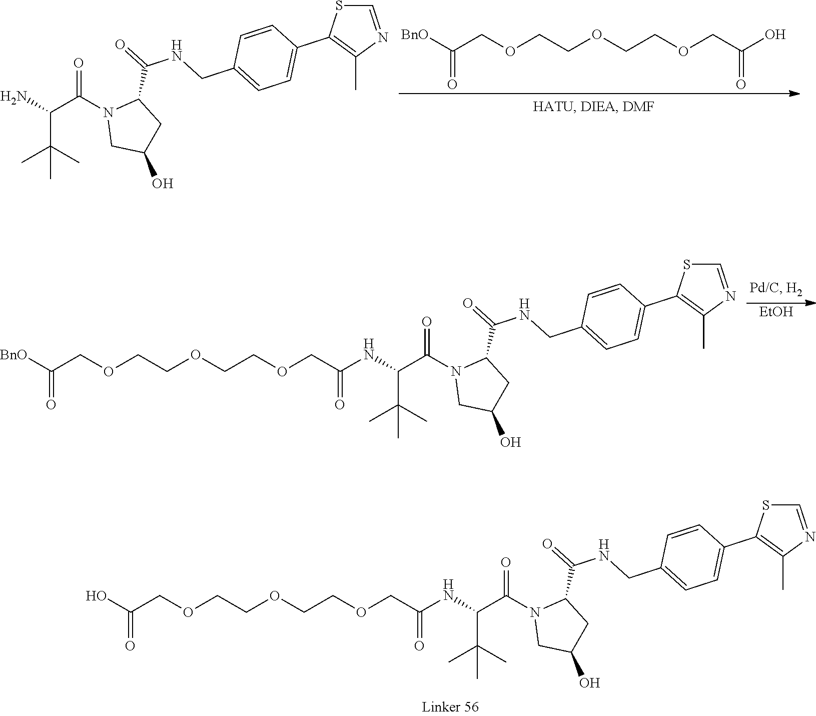

Linker 56 was synthesized following the same procedures as Linker 45 as described for Example 45. (1.21 g, yield 42%). ¹H NMR (400 MHz, CDCl₃) δ 8.68 (s, 1H), 7.80-7.71 (m, 11H), 7.41-7.33 (m, 5H), 4.71-7.65 (m, 1H), 4.61-4.50 (m, 3H), 4.37-4.33 (m, 1H), 4.07-3.94 (m, 5H), 3.77-3.58 (m, 10H), 2.51 (s, 3H), 2.38-2.30 (m, 1H), 2.24-2.19 (m, 1H), 0.98 (s, 9H). LCMS (ESI) m/z=635.0 [M+H]⁺.

### Example 57: (S)-18-((2S,4R)-4-Hydroxy-2-((4-(4-methylthiazol-5-yl)benzyl)carbamoyl)pyrrolidine-1-carbonyl)-19,19-dimethyl-16-oxo-4,7,10,13-tetraoxa-17-azaicosanoic acid (Linker 57)

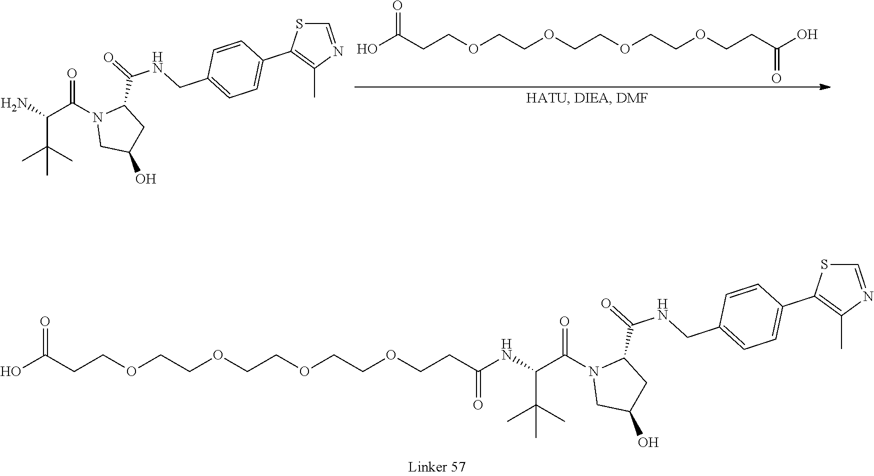

Linker 57 was synthesized following the same procedure as Linker 45 as described for Example 45. (1.6 g, yield 43%). ¹H NMR (400 MHz, CDCl₃) δ 8.69 (s, 1H), 7.55-7.52 (m, 1H), 7.47-7.45 (m, 1H), 7.36 (s, 4H), 4.70-4.66 (m, 1H), 4.62-4.57 (m, 2H), 4.50 (s, 1H), 4.34-4.29 (m, 1H), 4.12-4.09 (m, 1H), 3.75-3.48 (m, 18H), 2.56-2.47 (m, 7H), 2.40-2.33 (m, 1H), 2.23-2.18 (m, 1H), 0.96 (s, 9H). MS (ESI) m/z=707.1 [M+H]⁺.

### Example 58: (S)-21-((2S,4R)-4-Hydroxy-2-((4-(4-methylthiazol-5-yl)benzyl)carbamoyl)pyrrolidine-1-carbonyl)-22,22-dimethyl-19-oxo-4,7,10,13,16-pentaoxa-20-azatricosanoic acid (Linker 58)

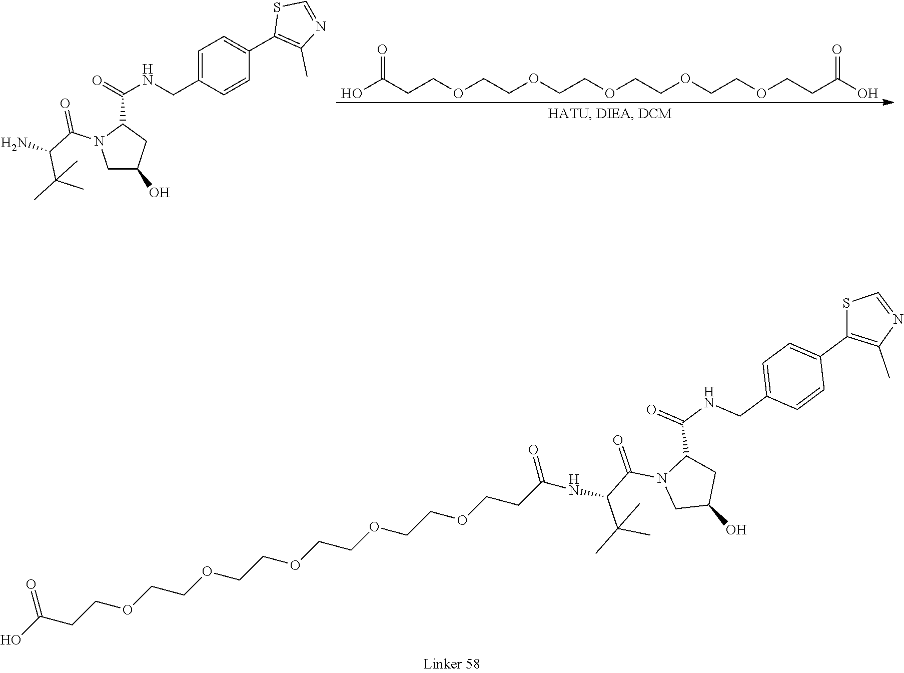

Linker 58 was synthesized following the same procedure as Linker 45 as described for Example 45. (1.2 g, yield: 23%). ¹H NMR (400 MHz, DMSO-d₆) δ8.98 (s, 1H), 8.57 (t, J=6.0 Hz, 1H), 7.91 (d, J=9.6 Hz, 1H), 7.43-7.31 (m, 4H), 4.56-4.53 (m, 1H), 4.45-4.35 (m, 3H), 4.24-4.19 (m, 1H), 3.69-3.55 (m, 6H), 3.49-3.47 (m, 16H), 2.57-2.53 (m, 1H), 2.45 (s, 3H), 2.39-2.32 (m, 3H), 2.06-2.01 (m, 1H), 1.93-1.86 (m, 1H), 0.95 (s, 9H). MS (ESI) m/z=751 [M+H]⁺.

### Example 59: (S)-19-((2S,4R)-4-Hydroxy-2-((4-(4-methylthiazol-5-yl)benzyl)carbamoyl)pyrrolidine-1-carbonyl)-20,20-dimethyl-17-oxo-3,6,9,12,15-pentaoxa-18-azahenicosanoic acid (Linker 59)

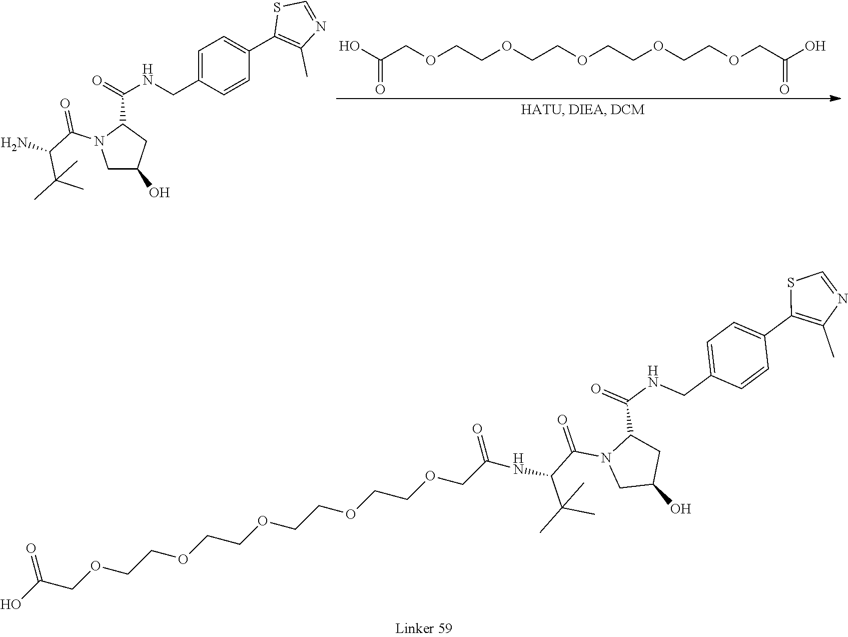

Linker 59 was synthesized following the same procedure as Linker 45 as described for Example 45. (1.3 g, yield: 39%). ¹H NMR (400 MHz, DMSO-d6) δ8.98 (s, 1H), 8.69 (t, J=6.0 Hz, 1H), 7.45 (d, J=9.6 Hz, 1H), 7.43-7.37 (m, 4H), 4.57-4.55 (m, 1H), 4.47-4.34 (m, 3H), 4.27-4.22 (m, 1H), 3.97 (s, 2H), 3.68-3.65 (m, 2H), 3.61-3.48 (m, 18H), 2.45 (s, 3H), 2.09-2.04 (m, 1H), 1.92-1.86 (m, 1H), 0.94 (s, 9H). MS (ESI) m/z=723 [M+H]⁺.

### Example 60: 5-((2-(2-Aminoethoxy)ethyl)amino)-2-(2,6-dioxopiperidin-3-yl)isoindoline-1,3-dione (Linker 60)

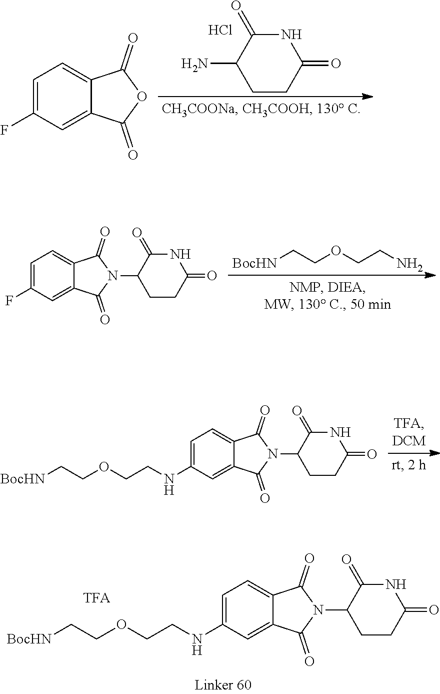

A mixture of 5-fluoroisobenzofuran-1,3-dione (87 g, 524 mmol), 3-aminopiperidine-2,6-dione (85.7 g, 524 mmol) and NaOAc (85.9 g, 1050 mmol) in acetic acid (500 mL) was stirred at 130° C. overnight. After cooling down to room temperature, the mixture was concentrated. The resulting residue was poured into ice water, and filtered. The filter cake was washed with water (500 mL×2), EtOH (500 mL×2), MeOH (500 mL) and DCM (500 mL) to afford a solid which was dried in vacuum to give 2-(2,6-dioxopiperidin-3-yl)-5-fluoroisoindoline-1,3-dione (120 g, yield: 83%) as yellow solid. MS (ESI) m/z=277.1 [M+H]⁺.

A mixture of 2-(2,6-dioxopiperidin-3-yl)-5-fluoroisoindoline-1,3-dione (6.9 g, 25.0 mmol), tert-butyl (2-(2-aminoethoxy)ethyl)carbamate (5.6 g, 27.5 mmol) and DIEA (9.7 g, 75 mmol) in NMP (75 mL) was stirred at 130° C. in microwave reactor for 50 min. After cooling down to room temperature, the mixture was poured into EtOAc (200 mL), and washed with water (200 mL×2) followed by brine (200 mL). The organic phase was dried over anhydrous Na₂SO₄, filtered and concentrated to give a crude product which was purified by chromatography on silica gel (petroleum ether:EtOAc=2:1 to 1:2) to give tert-butyl (2-(2-((2-(2,6-dioxopiperidin-3-yl)-1,3-dioxoisoindolin-5-yl)amino)ethoxy) ethyl)carbamate (2.4 g, yield: 21%) as yellow oil. MS (ESI) m/z=361.1 [M+H]⁺.

To a solution of tert-butyl (2-(2-((2-(2,6-dioxopiperidin-3-yl)-1,3-dioxoisoindolin-5-yl)amino)ethoxy)ethyl)carbamate (2.4 g, 5.2 mmol) in DCM (10 mL) was added TFA (5 mL) in one portion. The reaction mixture was stirred at room temperature for 2 h. After concentration, the resulting residue was dissolved in water (20 mL), washed with EtOAc (40 mL) and MTBE (40 mL). The aqueous phase was lyophilized to afford TFA salt of 5-((2-(2-aminoethoxy)ethyl)amino)-2-(2,6-dioxopiperidin-3-yl) isoindoline-1,3-dione (1.9 g, yield: 77%) as yellow solid. ¹H NMR (400 MHz, DMSO-d₆) δ 11.06 (s, 1H), 8.01 (s, 3H), 7.58 (d, J=8.4 Hz, 1H), 7.12 (br, s, 1H), 7.02 (d, J=2.0 Hz, 1H), 6.91 (dd, J=2.0 Hz, 8.8 Hz, 1H), 5.04 (dd, J=5.6 Hz, 13.2 Hz, 1H), 3.64 (t, J=5.6 Hz, 4H), 3.40 (t, J=5.2 Hz, 2H), 3.01 (br, 2H), 2.89-2.83 (m, 1H), 2.60-2.50 (m, 2H), 2.03-1.97 (m, 1H). MS (ESI) m/z=361.1 [M+H]⁺.

### Example 61: 5-((2-(2-(2-Aminoethoxy)ethoxy)ethyl)amino)-2-(2,6-dioxopiperidin-3-yl)isoindoline-1,3-dione (Linker 61)

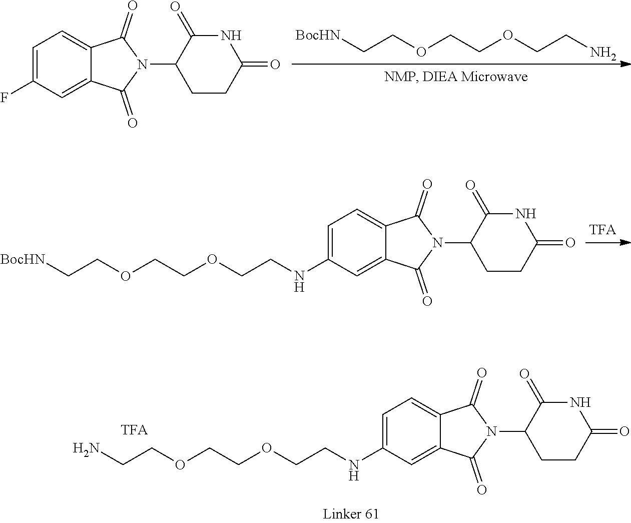 [SMILES: O=C1CCC(N2C(=O)c3ccc(F)cc3C2=O)C(=O)N1 ; CC(C)(C)OC(=O)NCCOCCOCCN ; CC(C)(C)OC(=O)NCCOCCOCCNc1ccc2c(c1)C(=O)N(C1CCC(=O)NC1=O)C2=O ; NCCOCCOCCNc1ccc2c(c1)C(=O)N(C1CCC(=O)NC1=O)C2=O]

Linker 61 was synthesized following the same procedure as Linker 60 as described for Example 60. (1.4 g, yield: 71%). ¹H NMR (400 MHz, DMSO-d₆) δ 11.05 (s, 1H), 7.94 (br, 3H), 7.56 (d, J=8.4 Hz, 1H), 7.01 (s, 1H), 6.90 (d, J=8.0 Hz, 1H), 5.03 (dd, J=5.2 Hz, 12.8 Hz, 1H), 3.58 (br, 8H), 3.36 (s, 2H), 2.97-2.92 (m, 2H), 2.91-2.83 (m, 1H), 2.60-2.50 (m, 2H), 2.01-1.99 (m, 1H). MS (ESI) m/z=405.1 [M+H]⁺.

### Example 62: 5-((2-(2-(2-(2-Aminoethoxy)ethoxy)ethoxy)ethyl)amino)-2-(2,6-dioxopiperidin-3-yl)isoindoline-1,3-dione (Linker 62)

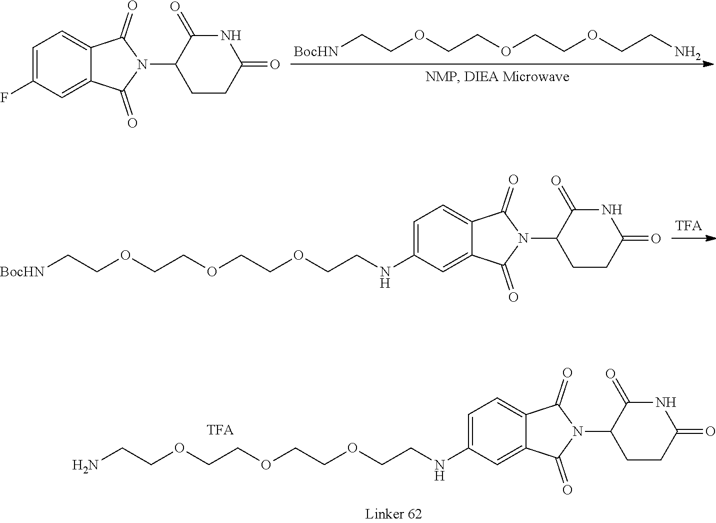 [SMILES: O=C1CCC(N2C(=O)c3ccc(F)cc3C2=O)C(=O)N1 ; CC(C)(C)OC(=O)NCCOCCOCCOCCN ; CC(C)(C)OC(=O)NCCOCCOCCOCCNc1ccc2c(c1)C(=O)N(C1CCC(=O)NC1=O)C2=O ; NCCOCCOCCOCCNc1ccc2c(c1)C(=O)N(C1CCC(=O)NC1=O)C2=O]

Linker 62 was synthesized following the same procedure as Linker 60 as described for Example 60. (1.19 g, yield: 59%). ¹H NMR (400 MHz, DMSO-d₆) δ 11.05 (s, 1H), 7.79 (br, 3H), 7.57 (d, J=8.4 Hz, 1H), 7.15 (br, s, 1H), 7.00 (d, J=2.0 Hz, 1H), 6.90 (dd, J=2.0 Hz, 8.4 Hz, 1H), 5.03 (dd, J=5.6 Hz, 12.8 Hz, 1H), 3.61-3.55 (m, 12H), 3.36 (t, J=5.6 Hz, 2H), 2.99-2.94 (m, 2H), 2.88-2.84 (m, 1H), 2.60-2.52 (m, 2H) 2.01-1.98 (m, 1H). MS (ESI) m/z=449.1 [M+H]⁺.

### Example 63: 5-((14-Amino-3,6,9,12-tetraoxatetradecyl)amino)-2-(2,6-dioxopiperidin-3-yl)isoindoline-1,3-dione (Linker 63)

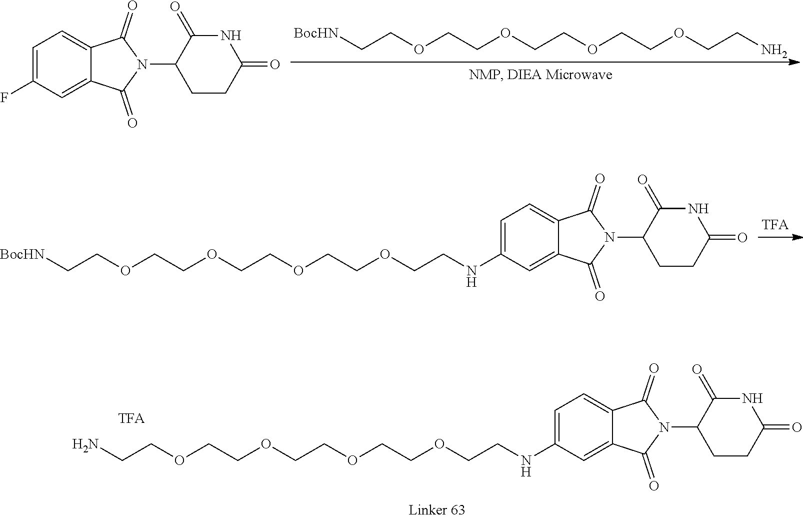 [SMILES: O=C1CCC(N2C(=O)c3ccc(F)cc3C2=O)C(=O)N1 ; CC(C)(C)OC(=O)NCCOCCOCCOCCOCCN ; CC(C)(C)OC(=O)NCCOCCOCCOCCOCCNc1ccc2c(c1)C(=O)N(C1CCC(=O)NC1=O)C2=O ; NCCOCCOCCOCCOCCNc1ccc2c(c1)C(=O)N(C1CCC(=O)NC1=O)C2=O]

Linker 63 was synthesized following the same procedure as Linker 60 as described for Example 60. (1.2 g, yield: 73%). ¹H NMR (400 MHz, DMSO-d₆) δ 11.05 (s, 1H), 7.79 (br, J=1.6 Hz, 3H), 7.56 (d, J=8.4 Hz, 1H), 7.14 (br, s, 1H), 7.01 (d, J=2.0 Hz, 1H), 6.90 (dd, J=2.0 Hz, 8.4 Hz, 1H), 5.03 (dd, J=5.6 Hz, 13.2 Hz, 1H), 3.61-3.56 (m, 16H), 3.36 (t, J=5.2 Hz, 2H), 2.99-2.95 (m, 2H), 2.89-2.83 (m, 1H), 2.60-2.53 (m, 2H) 2.01-1.97 (m, 1H). MS (ESI) m/z=493.1 [M+H]⁺.

### Example 64: 5-((17-Amino-3,6,9,12,15-pentaoxaheptadecyl)amino)-2-(2,6-dioxopiperidin-3-yl)isoindoline-1,3-dione (Linker 64)

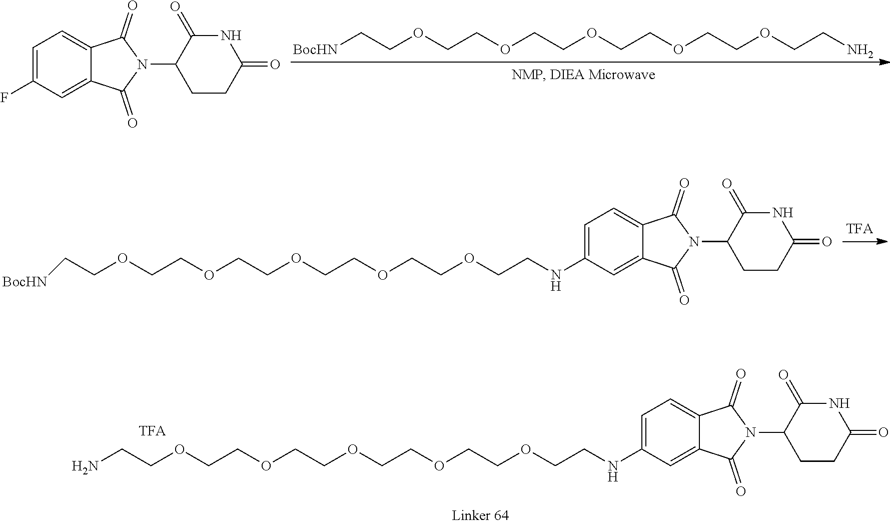 [SMILES: O=C1CCC(N2C(=O)c3ccc(F)cc3C2=O)C(=O)N1 ; CC(C)(C)OC(=O)NCCOCCOCCOCCOCCOCCN ; CC(C)(C)OC(=O)NCCOCCOCCOCCOCCOCCNc1ccc2c(c1)C(=O)N(C1CCC(=O)NC1=O)C2=O ; NCCOCCOCCOCCOCCOCCNc1ccc2c(c1)C(=O)N(C1CCC(=O)NC1=O)C2=O]

Linker 64 was synthesized following the same procedure as Linker 60 as described for Example 60. (1.73 g, yield: 88%). ¹H NMR (400 MHz, DMSO-d₆) δ 11.05 (s, 1H), 7.79 (s, 3H), 7.55 (d, J=8.4 Hz, 1H), 7.18 (br, s, 1H), 7.01 (s, 1H), 6.90 (d, J=8.4 Hz, 1H), 5.03 (dd, J=5.2 Hz, 12.8 Hz, 1H), 3.61-3.54 (m, 20H), 3.35 (s, 2H), 2.98 (s, 2H), 2.92-2.83 (m, 1H), 2.61-2.54 (m, 2H), 2.02-1.98 (m, 1H). MS (ESI) m/z=537.2 [M+H]⁺.

### Example 65: (2-(2,6-Dioxopiperidin-3-yl)-1,3-dioxoisoindolin-5-yl)glycine (Linker 65)

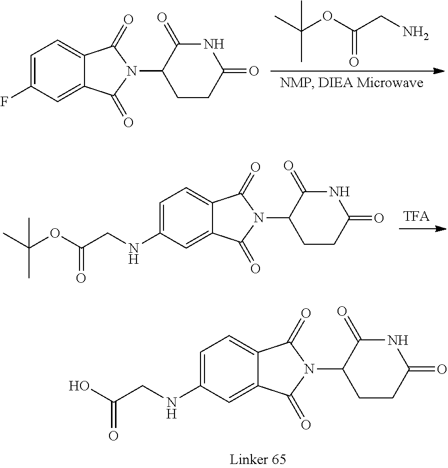

Linker 65 was synthesized following the same procedure as Linker 60 as described for Example 60. (1.0 g, yield: 84%). ¹H NMR (400 MHz, DMSO-d₆) δ 12.80 (br, 1H), 11.06 (s, 1H), 7.59 (d, J=8.4 Hz, 1H), 7.32 (br, s, 1H), 6.98 (d, J=1.2 Hz, 1H), 6.89 (dd, J=2.0 Hz, 8.4 Hz, 1H), 5.04 (dd, J=5.6 Hz, 13.2 Hz, 1H), 4.03 (s, 2H), 2.92-2.83 (m, 1H), 2.60-2.52 (m, 2H), 2.03-1.98 (m, 1H). MS (ESI) m/z=332.0 [M+H]⁺.

### Example 66: 3-((2-(2,6-Dioxopiperidin-3-yl)-1,3-dioxoisoindolin-5-yl)amino)propanoic acid (Linker 66)

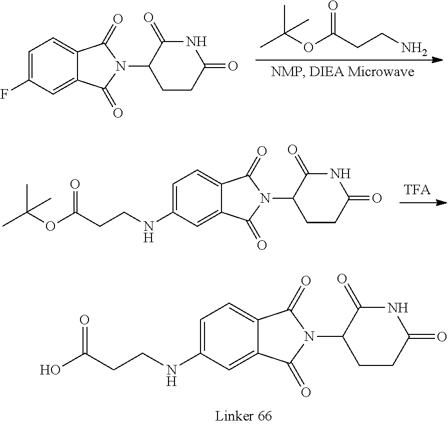

Linker 66 was synthesized following the same procedure as Linker 60 as described for Example 60. (1.24 g, yield: 60%). ¹H NMR (400 MHz, DMSO-d₆) δ 11.05 (s, 1H), 7.57 (d, J=8.4 Hz, 1H), 6.97 (d, J=2.0 Hz, 1H), 6.87 (dd, J=2.0 Hz, 8.4 Hz, 1H), 5.02 (dd, J=5.2 Hz, 12.8 Hz, 1H), 3.41 (t, J=6.8 Hz, 2H), 2.89-2.83 (m, 1H), 2.60-2.52 (m, 4H), 2.02-1.97 (m, 1H). MS (ESI) m/z=346.0 [M+H]⁺.

### Example 67: 4-((2-(2,6-Dioxopiperidin-3-yl)-1,3-dioxoisoindolin-5-yl)amino)butanoic acid (Linker 67)

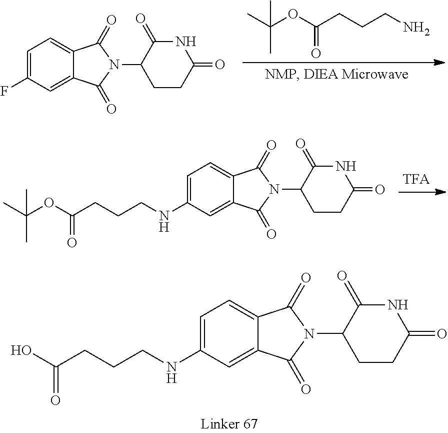 [SMILES: O=C1CCC(N2C(=O)c3ccc(F)cc3C2=O)C(=O)N1 ; CC(C)(C)OC(=O)CCCN ; CC(C)(C)OC(=O)CCCNc1ccc2c(c1)C(=O)N(C1CCC(=O)NC1=O)C2=O ; O=C(O)CCCNc1ccc2c(c1)C(=O)N(C1CCC(=O)NC1=O)C2=O]

Linker 67 was synthesized following the same procedure as Linker 60 as described for Example 60. (0.52 g, yield: 25%). ¹H NMR (400 MHz, DMSO-d₆) δ 12.12 (s, 1H), 11.05 (s, 1H), 7.55 (d, J=8.4 Hz, 1H), 7.14 (t, J=4.8 Hz, 1H), 6.95 (d, J=2.0 Hz, 1H), 6.85 (dd, J=2.0 Hz, 8.4 Hz, 1H), 5.02 (dd, J=5.6 Hz, 12.8 Hz, 1H), 3.21-3.16 (m, 2H), 2.91-2.83 (m, 1H), 2.60-2.51 (m, 2H), 2.34 (t, J=7.2 Hz, 2H), 2.01-1.97 (m, 1H), 1.82-1.75 (m, 2H). MS (ESI) m/z=360.1 [M+H]⁺.

### Example 68: 5-((2-(2,6-Dioxopiperidin-3-yl)-1,3-dioxoisoindolin-5-yl)amino)pentanoic acid (Linker 68)

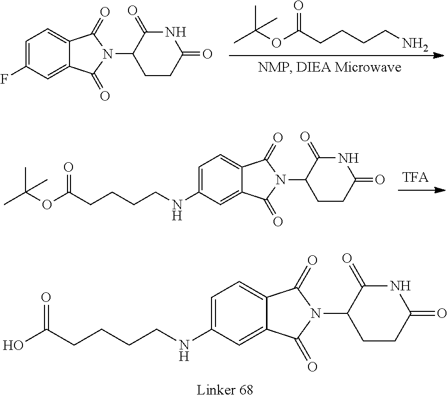

Linker 68 was synthesized following the same procedure as Linker 60 as described for Example 60. (0.66 g, yield: 51%). ¹H NMR (400 MHz, DMSO-d₆) δ 12.03 (br, 1H), 11.05 (s, 1H), 7.55 (d, J=8.4 Hz, 1H), 7.10 (t, J=5.2 Hz, 1H), 6.94 (s, 1H), 6.83 (dd, J=1.6 Hz, 8.4 Hz, 1H), 5.02 (dd, J=5.6 Hz, 12.8 Hz, 1H), 3.17-3.16 (m, 2H), 2.92-2.83 (m, 1H), 2.60-2.53 (m, 2H), 2.26-2.25 (m, 2H), 2.01-1.98 (m, 1H), 1.60-1.59 (m, 4H). MS (ESI) m/z=374.1 [M+H]⁺.

### Example 69: 6-((2-(2,6-Dioxopiperidin-3-yl)-1,3-dioxoisoindolin-5-yl)amino)hexanoic acid (Linker 69)

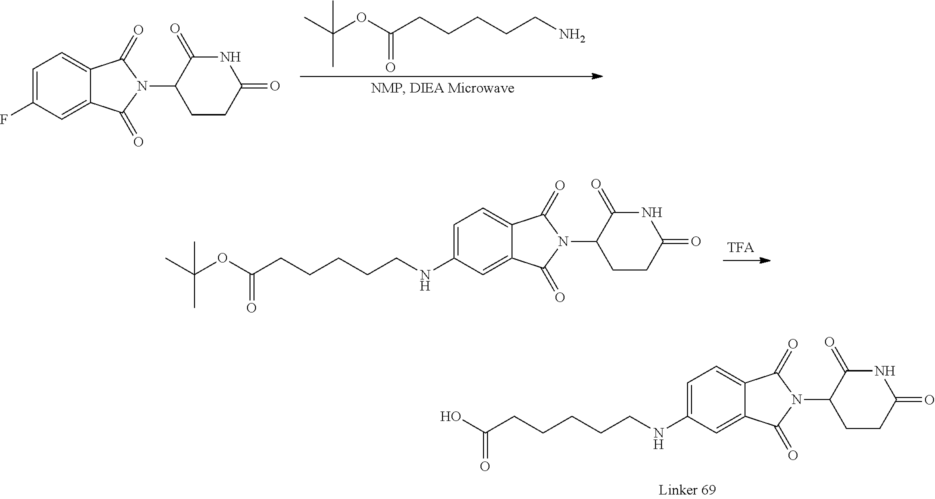 [SMILES: O=C1CCC(N2C(=O)c3ccc(F)cc3C2=O)C(=O)N1 ; CC(C)(C)OC(=O)CCCCCN ; CC(C)(C)OC(=O)CCCCCNc1ccc2c(c1)C(=O)N(C1CCC(=O)NC1=O)C2=O ; O=C(O)CCCCCNc1ccc2c(c1)C(=O)N(C1CCC(=O)NC1=O)C2=O]

Linker 69 was synthesized following the same procedure as Linker 60 as described for Example 60. (1.33 g, yield: 66%). ¹H NMR (400 MHz, DMSO-d₆) δ 11.98 (s, 1H), 11.05 (s, 1H), 7.55 (d, J=8.4 Hz, 1H), 7.08 (t, J=5.2 Hz, 1H), 6.95 (s, 1H), 6.83 (dd, J=1.2 Hz, 8.4 Hz, 1H), 5.03 (dd, J=5.2 Hz, 12.8 Hz, 1H), 3.17-3.12 (m, 2H), 2.92-2.83 (m, 1H), 2.60-2.53 (m, 2H), 2.22 (t, J=7.2 Hz, 2H), 2.01-1.98 (m, 1H), 1.61-1.51 (m, 4H), 1.41-1.33 (m, 2H). MS (ESI) m/z=388.1 [M+H]⁺.

### Example 70: 7-((2-(2,6-Dioxopiperidin-3-yl)-1,3-dioxoisoindolin-5-yl)amino)heptanoic acid (Linker 70)

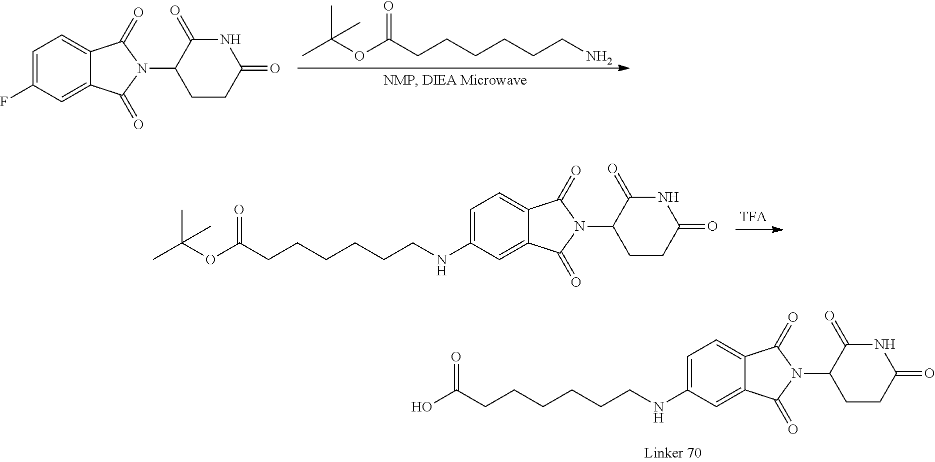

Linker 70 was synthesized following the same procedure as Linker 60 as described for Example 60. (1.06 g, yield: 39%). ¹H NMR (400 MHz, DMSO-d₆) δ 11.94 (s, 1H), 11.04 (s, 1H), 7.55 (d, J=8.4 Hz, 1H), 7.09 (t, J=5.6 Hz, 1H), 6.94 (d, J=2.0 Hz, 1H), 6.84 (dd, J=2.0 Hz, 8.4 Hz, 1H), 5.02 (dd, J=5.6 Hz, 13.2 Hz, 1H), 3.17-3.12 (m, 2H), 2.88-2.83 (m, 1H), 2.60-2.53 (m, 2H), 2.21 (t, J=7.2 Hz, 2H), 2.01-1.97 (m, 1H), 1.58-1.48 (m, 4H), 1.39-1.29 (m, 4H). MS (ESI) m/z=402.1 [M+H]⁺.

### Example 71: 8-((2-(2,6-Dioxopiperidin-3-yl)-1,3-dioxoisoindolin-5-yl)amino)octanoic acid (Linker 71)

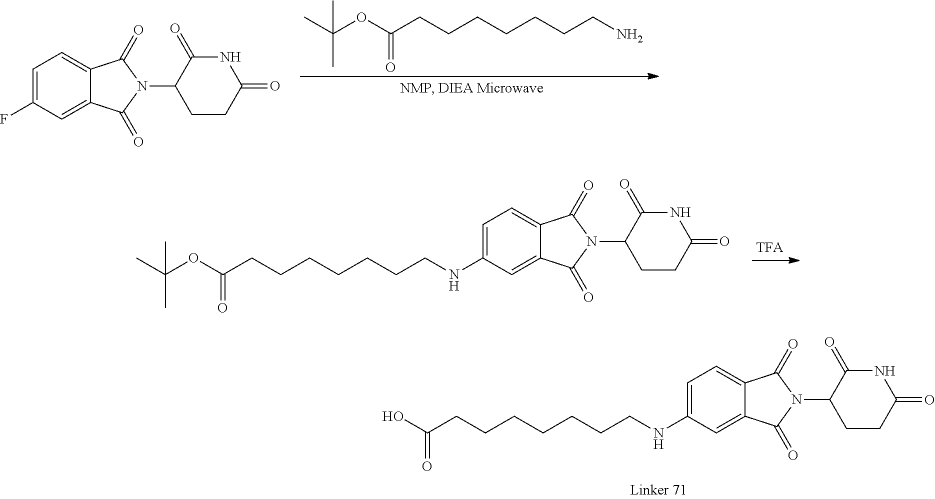 [SMILES: O=C1CCC(N2C(=O)c3ccc(F)cc3C2=O)C(=O)N1 ; CC(C)(C)OC(=O)CCCCCCCN ; CC(C)(C)OC(=O)CCCCCCCNc1ccc2c(c1)C(=O)N(C1CCC(=O)NC1=O)C2=O ; O=C(O)CCCCCCCNc1ccc2c(c1)C(=O)N(C1CCC(=O)NC1=O)C2=O]

Linker 71 was synthesized following the same procedure as Linker 60 as described for Example 60. (1.66 g, yield: 51%). ¹H NMR (400 MHz, DMSO-d₆) δ 11.95 (s, 1H), 11.05 (s, 1H), 7.55 (d, J=8.4 Hz, 1H), 7.09 (t, J=5.6 Hz, 1H), 6.94 (d, J=2.0 Hz, 1H), 6.84 (dd, J=2.0 Hz, 8.4 Hz, 1H), 5.02 (dd, J=5.6 Hz, 13.2 Hz, 1H), 3.17-3.12 (m, 2H), 2.88-2.83 (m, 1H), 2.60-2.53 (m, 2H), 2.19 (t, J=7.2 Hz, 2H), 2.02-1.98 (m, 1H), 1.58-1.47 (m, 4H), 1.36-1.29 (m, 6H). MS (ESI) m/z=416.1 [M+H]⁺.

### Example 72: 5-((2-Aminoethyl)amino)-2-(2,6-dioxopiperidin-3-yl)isoindoline-1,3-dione (Linker 72)

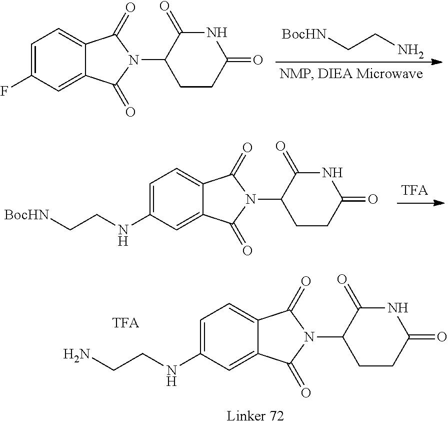 [SMILES: O=C1CCC(N2C(=O)c3ccc(F)cc3C2=O)C(=O)N1 ; CC(C)(C)OC(=O)NCCN ; CC(C)(C)OC(=O)NCCNc1ccc2c(c1)C(=O)N(C1CCC(=O)NC1=O)C2=O ; NCCNc1ccc2c(c1)C(=O)N(C1CCC(=O)NC1=O)C2=O]

Linker 72 was synthesized following the same procedure as Linker 60 as described for Example 60. (1.74 g, yield: 80%). ¹H NMR (400 MHz, DMSO-d₆) δ 11.08 (s, 1H), 8.10 (s, 3H), 7.62 (d, J=8.4 Hz, 1H), 7.33 (t, J=5.2 Hz, 1H), 7.05 (s, 1H), 6.94 (d, J=8.0 Hz, 1H), 5.07 (dd, J=5.2 Hz, 12.8 Hz, 1H), 3.50-3.49 (m, 2H), 3.03 (t, J=6.0 Hz, 2H), 2.95-2.86 (m, 1H), 2.63-2.57 (m, 2H), 2.05-2.02 (m, 1H). MS (ESI) m/z=317.1 [M+H]⁺.

### Example 73: 5-((3-Aminopropyl)amino)-2-(2,6-dioxopiperidin-3-yl)isoindoline-1,3-dione (Linker 73)

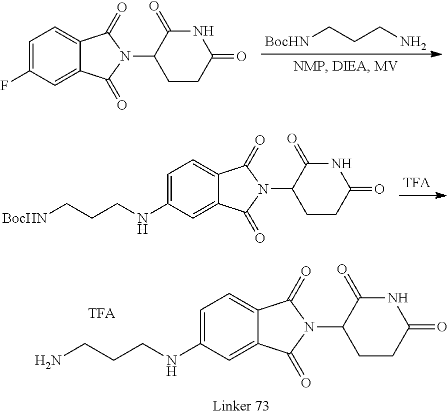 [SMILES: O=C1CCC(N2C(=O)c3ccc(F)cc3C2=O)C(=O)N1 ; CC(C)(C)OC(=O)NCCCN ; CC(C)(C)OC(=O)NCCCNc1ccc2c(c1)C(=O)N(C1CCC(=O)NC1=O)C2=O ; NCCCNc1ccc2c(c1)C(=O)N(C1CCC(=O)NC1=O)C2=O]

Linker 73 was synthesized following the same procedure as Linker 60 as described for Example 60. (1.3 g, yield: 57%). ¹H NMR (400 MHz, DMSO-d₆) δ 11.07 (s, 1H), 7.85 (br, 3H), 7.59 (d, J=8.4 Hz, 1H), 7.22 (t, J=5.2 Hz, 1H), 6.98 (d, J=2.0 Hz, 1H), 6.88 (dd, J=2.0 Hz, 8.4 Hz, 1H), 5.04 (dd, J=5.6 Hz, 13.2 Hz, 1H), 3.29-3.25 (m, 2H), 2.91-2.85 (m, 3H), 2.60-2.53 (m, 2H), 2.02-1.98 (m, 1H), 1.87-1.81 (m, 2H). MS (ESI) m/z=331.1 [M+H]⁺.

### Example 74: 5-((4-Aminobutyl)amino)-2-(2,6-dioxopiperidin-3-yl)isoindoline-1,3-dione (Linker 74)

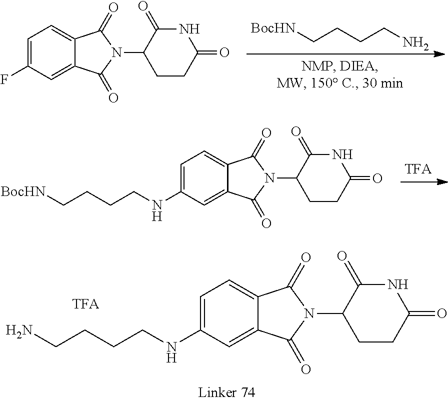 [SMILES: O=C1CCC(N2C(=O)c3ccc(F)cc3C2=O)C(=O)N1 ; CC(C)(C)OC(=O)NCCCCN ; CC(C)(C)OC(=O)NCCCCNc1ccc2c(c1)C(=O)N(C1CCC(=O)NC1=O)C2=O ; NCCCCNc1ccc2c(c1)C(=O)N(C1CCC(=O)NC1=O)C2=O]

Linker 74 was synthesized following the same procedure as Linker 60 as described for Example 60. (2.9 g, yield: 85%). ¹H NMR (400 MHz, DMSO-d₆) δ 11.08 (s, 1H), 7.97 (br, 3H), 7.58 (d, J=8.4 Hz, 1H), 7.22 (br, s, 1H), 6.99 (s, 1H), 6.89 (d, J=8.0 Hz, 1H), 5.05 (dd, J=5.2 Hz, 12.8 Hz, 1H), 3.22 (s, 2H), 2.93-2.84 (m, 3H), 2.63-2.53 (m, 2H), 2.04-2.00 (m, 1H), 1.66 (s, 4H). MS (ESI) m/z=345.1 [M+H]⁺.

### Example 75: 5-((5-Aminopentyl)amino)-2-(2,6-dioxopiperidin-3-yl)isoindoline-1,3-dione (Linker 75)

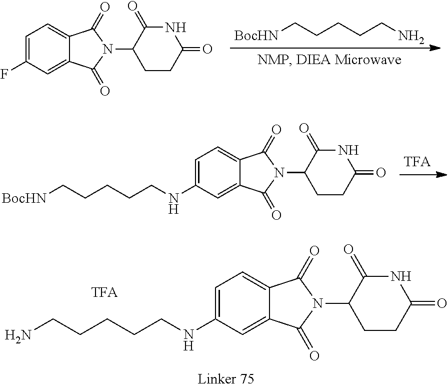 [SMILES: O=C1CCC(N2C(=O)c3ccc(F)cc3C2=O)C(=O)N1 ; CC(C)(C)OC(=O)NCCCCCN ; CC(C)(C)OC(=O)NCCCCCNc1ccc2c(c1)C(=O)N(C1CCC(=O)NC1=O)C2=O ; NCCCCCNc1ccc2c(c1)C(=O)N(C1CCC(=O)NC1=O)C2=O]

Linker 75 was synthesized following the same procedure as Linker 60 as described for Example 60. (1.8 g, yield: 78%). ¹H NMR (400 MHz, DMSO-d₆) δ 11.09 (s, 1H), 7.89 (br, 3H), 7.57 (d, J=6.8 Hz, 1H), 7.17 (br, s, 1H), 6.96 (s, 1H), 6.86 (d, J=6.0 Hz, 1H), 5.05 (d, J=7.2 Hz, 1H), 3.19-3.15 (m, 2H), 2.89-2.70 (m, 3H), 2.61-2.51 (m, 2H), 2.01-1.90 (m, 1H), 1.62-1.56 (m, 4H), 1.45-1.40 (m, 2H). MS (ESI) m/z=359.1 [M+H]⁺.

### Example 76: 5-((6-Aminohexyl)amino)-2-(2,6-dioxopiperidin-3-yl)isoindoline-1,3-dione (Linker 76)

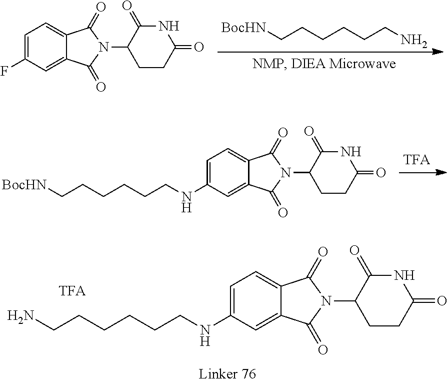 [SMILES: O=C1CCC(N2C(=O)c3ccc(F)cc3C2=O)C(=O)N1 ; CC(C)(C)OC(=O)NCCCCCCN ; CC(C)(C)OC(=O)NCCCCCCNc1ccc2c(c1)C(=O)N(C1CCC(=O)NC1=O)C2=O ; NCCCCCCNc1ccc2c(c1)C(=O)N(C1CCC(=O)NC1=O)C2=O]

Linker 76 was synthesized following the same procedure as Linker 60 as described for Example 60. (1.8 g, yield: 62%). ¹H NMR (400 MHz, DMSO-d6) δ 11.05 (s, 1H), 7.71 (br, 3H), 7.57 (d, J=8.4 Hz, 1H), 7.12 (t, J=5.2 Hz, 1H), 6.94 (d, J=2.0 Hz, 1H), 6.85 (dd, J=2.0 Hz, 8.4 Hz, 1H), 5.03 (dd, J=5.2 Hz, 12.8 Hz, 1H), 3.17-3.16 (m, 2H), 2.88-2.77 (m, 3H), 2.60-2.53 (m, 2H), 2.01-1.98 (m, 1H), 1.59-1.51 (m, 4H), 1.37-1.36 (m, 4H). MS (ESI) m/z=373.1 [M+H]⁺.

### Example 77: 5-((7-Aminoheptyl)amino)-2-(2,6-dioxopiperidin-3-yl)isoindoline-1,3-dione (Linker 77)

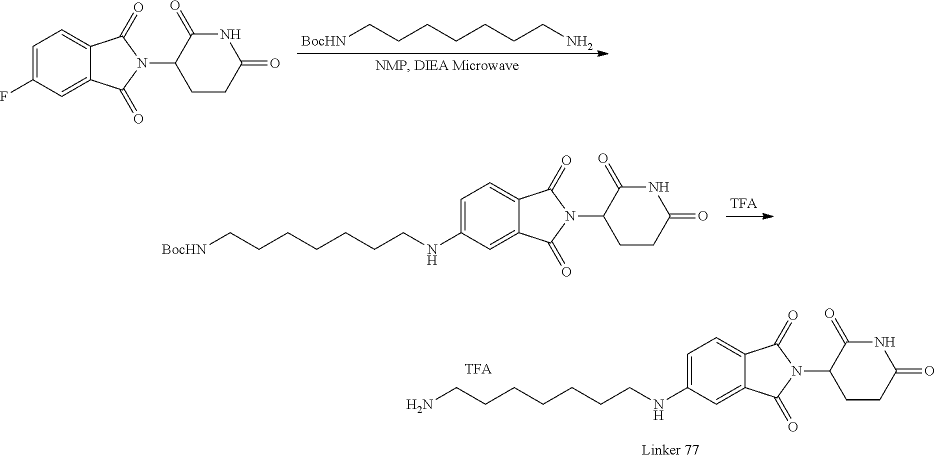 [SMILES: O=C1CCC(N2C(=O)c3ccc(F)cc3C2=O)C(=O)N1 ; CC(C)(C)OC(=O)NCCCCCCCN ; CC(C)(C)OC(=O)NCCCCCCCNc1ccc2c(c1)C(=O)N(C1CCC(=O)NC1=O)C2=O ; NCCCCCCCNc1ccc2c(c1)C(=O)N(C1CCC(=O)NC1=O)C2=O]

Linker 77 was synthesized following the same procedure as Linker 60 as described for Example 60. (1.3 g, yield: 70%). ¹H NMR (400 MHz, DMSO-d₆) δ 11.05 (s, 1H), 7.72 (br, 3H), 7.56 (d, J=8.4 Hz, 1H), 7.12 (t, J=5.6 Hz, 1H), 6.94 (d, J=2.0 Hz, 1H), 6.85 (dd, J=2.4 Hz, 8.8 Hz, 1H), 5.03 (dd, J=5.6 Hz, 12.8 Hz, 1H), 3.18-3.14 (m, 2H), 2.92-2.76 (m, 3H), 2.60-2.51 (m, 2H), 2.01-1.98 (m, 1H), 1.59-1.51 (m, 4H), 1.36-1.32 (m, 6H). MS (ESI) m/z=387.1 [M+H]⁺.

### Example 78: 5-((8-Aminooctyl)amino)-2-(2,6-dioxopiperidin-3-yl)isoindoline-1,3-dione (Linker 78)

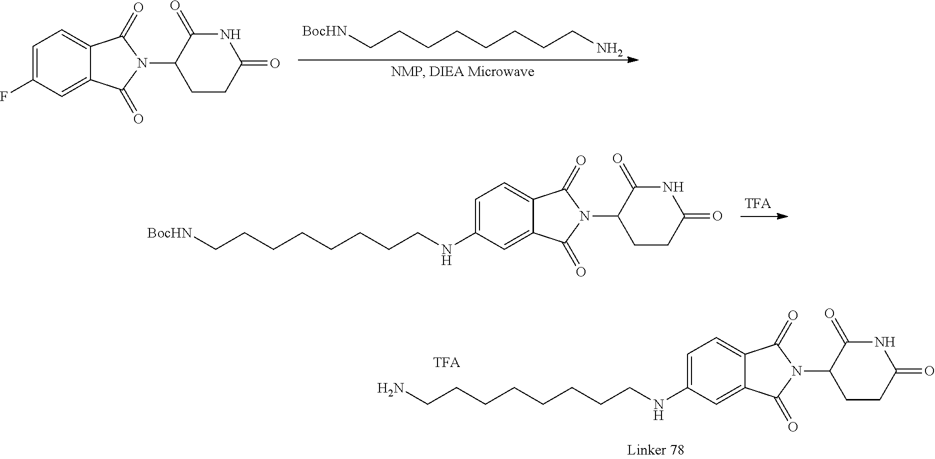 [SMILES: O=C1CCC(N2C(=O)c3ccc(F)cc3C2=O)C(=O)N1 ; CC(C)(C)OC(=O)NCCCCCCCCN ; CC(C)(C)OC(=O)NCCCCCCCCNc1ccc2c(c1)C(=O)N(C1CCC(=O)NC1=O)C2=O ; NCCCCCCCCNc1ccc2c(c1)C(=O)N(C1CCC(=O)NC1=O)C2=O]

Linker 78 was synthesized following the same procedure as Linker 60 as described for Example 60. (1.6 g, yield: 62%). ¹H NMR (400 MHz, DMSO-d₆) δ 11.05 (s, 1H), 7.73 (br, 3H), 7.56 (d, J=8.4 Hz, 1H), 7.14 (br, 1H), 6.94 (d, J=1.6 Hz, 1H), 6.85 (dd, J=2.0 Hz, 8.8 Hz, 1H), 5.03 (dd, J=5.6 Hz, 12.8 Hz, 1H), 3.15 (t, J=7.2 Hz, 2H), 2.89-2.83 (m, 1H), 2.80-2.75 (m, 2H), 2.60-2.54 (m, 2H), 2.02-1.98 (m, 1H), 1.59-1.51 (m, 4H), 1.37-1.30 (m, 8H). MS (ESI) m/z=401.1 [M+H]⁺.

### Example 79: 3-(2-((2-(2,6-Dioxopiperidin-3-yl)-1,3-dioxoisoindolin-5-yl)amino)ethoxy)propanoic acid (Linker 79)

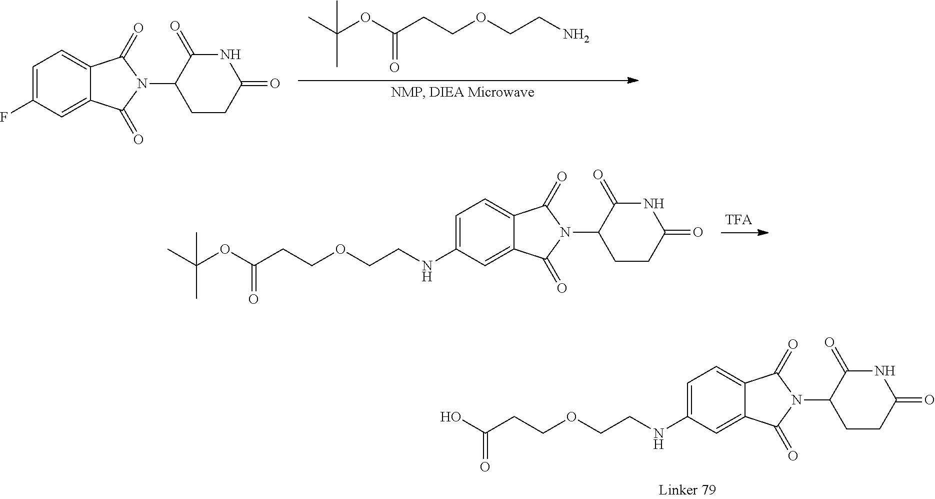 [SMILES: O=C1CCC(N2C(=O)c3ccc(F)cc3C2=O)C(=O)N1 ; CC(C)(C)OC(=O)CCOCCN ; CC(C)(C)OC(=O)CCOCCNc1ccc2c(c1)C(=O)N(C1CCC(=O)NC1=O)C2=O ; O=C(O)CCOCCNc1ccc2c(c1)C(=O)N(C1CCC(=O)NC1=O)C2=O]

Linker 79 was synthesized following the same procedure as Linker 60 as described for Example 60. (1.7 g, yield: 60%). ¹H NMR (400 MHz, DMSO-d₆) δ 12.19 (br, 1H), 11.06 (s, 1H), 7.57 (d, J=8.4 Hz, 1H), 7.09 (br, 1H), 7.01 (d, J=2.0 Hz, 1H), 6.90 (dd, J=2.0 Hz, 8.4 Hz, 1H), 5.04 (dd, J=5.6 Hz, 13.2 Hz, 1H), 3.66 (t, J=6.4 Hz, 2H), 3.59 (t, J=5.6 Hz, 2H), 3.35 (t, J=5.2 Hz, 2H), 2.93-2.84 (m, 1H), 2.62-2.56 (m, 2H), 2.52-2.47 (m, 2H), 2.03-1.99 (m, 1H). MS (ESI) m/z=390.1 [M+H]⁺.

### Example 80: 3-(2-(2-((2-(2,6-Dioxopiperidin-3-yl)-1,3-dioxoisoindolin-5-yl)amino)ethoxy)ethoxy)propanoic acid (Linker 80)

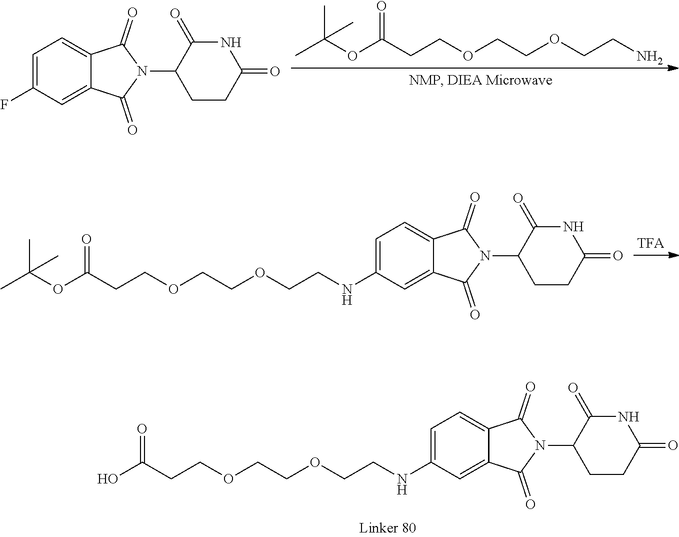

Linker 80 was synthesized following the same procedure as Linker 60 as described for Example 60. (2.3 g, yield: 78%). ¹H NMR (400 MHz, DMSO-d₆) δ 11.06 (s, 1H), 7.57 (d, J=8.4 Hz, 1H), 7.02 (d, J=2.0 Hz, 1H), 6.90 (dd, J=2.0 Hz, 8.4 Hz, 1H), 5.04 (dd, J=5.6 Hz, 13.2 Hz, 1H), 3.63-3.59 (m, 4H), 3.57-3.51 (m, 4H), 3.36 (t, J=5.6 Hz, 2H), 2.90-2.84 (m, 1H), 2.61-2.55 (m, 2H), 2.44 (t, J=6.4 Hz, 2H), 2.04-1.99 (m, 1H). MS (ESI) m/z=434.1 [M+H]⁺.

### Example 81: 3-(2-(2-(2-((2-(2,6-Dioxopiperidin-3-yl)-1,3-dioxoisoindolin-5-yl)amino)ethoxy)ethoxy)ethoxy)propanoic acid (Linker 81)

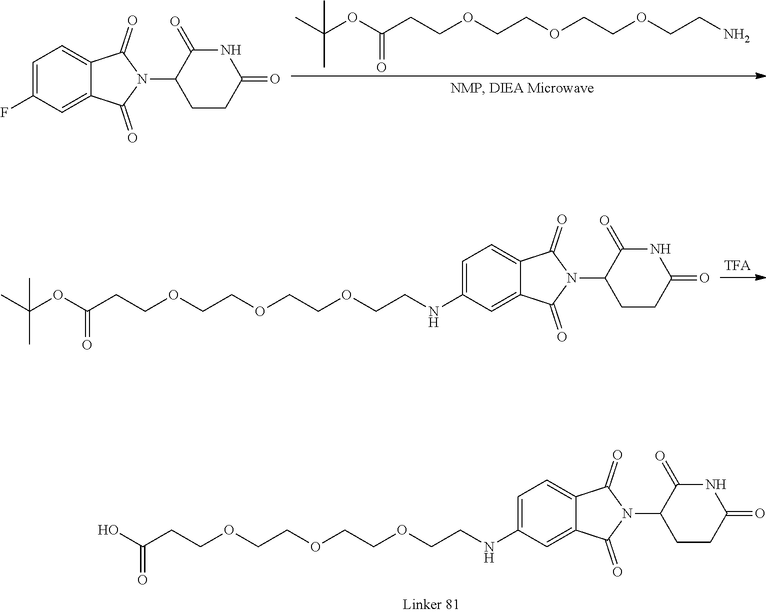 [SMILES: O=C1CCC(N2C(=O)c3ccc(F)cc3C2=O)C(=O)N1 ; CC(C)(C)OC(=O)CCOCCOCCOCCN ; CC(C)(C)OC(=O)CCOCCOCCOCCNc1ccc2c(c1)C(=O)N(C1CCC(=O)NC1=O)C2=O ; O=C(O)CCOCCOCCOCCNc1ccc2c(c1)C(=O)N(C1CCC(=O)NC1=O)C2=O]

Linker 81 was synthesized following the same procedure as Linker 60 as described for Example 60. (1.2 g, yield: 52%). ¹H NMR (400 MHz, DMSO-d₆) δ 7.59 (d, J=11.2 Hz, 1H), 7.23 (t, J=6.8 Hz, 1H), 7.04 (d, J=1.6 Hz, 1H), 7.04 (dd, J=2.4 Hz, 11.2 Hz, 1H), 5.06 (dd, J=7.2 Hz, 16.8 Hz, 1H), 3.64-3.57 (m, 8H), 3.54-3.48 (m, 4H), 3.40-3.38 (m, 2H), 2.92-2.89 (m, 1H), 2.64-2.54 (m, 2H), 2.42-2.38 (m, 2H), 2.05-2.01 (m, 1H). MS (ESI) m/z=478.1 [M+H]⁺.

### Example 82: 1-((2-(2,6-Dioxopiperidin-3-yl)-1,3-dioxoisoindolin-5-yl)amino)-3,6,9,12-tetraoxapentadecan-15-oic acid (Linker 82)

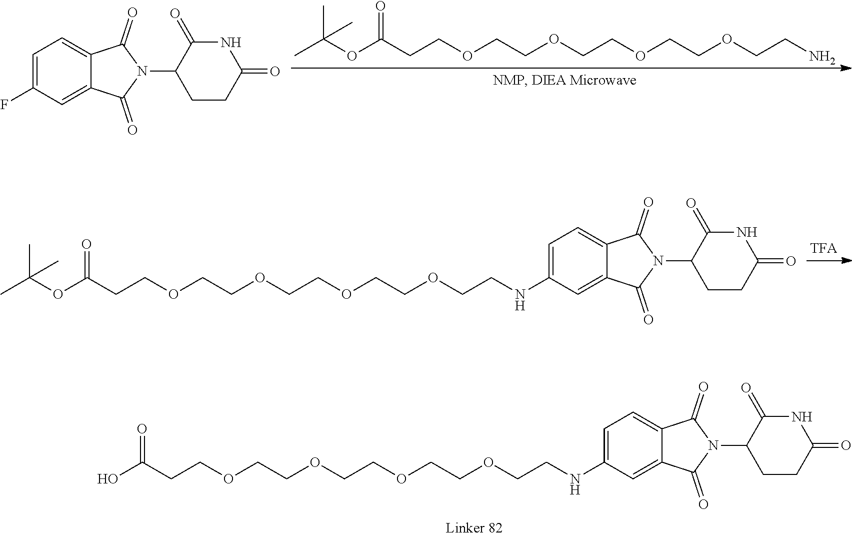

Linker 82 was synthesized following the same procedure as Linker 60 as described for Example 60. (1.3 g, yield: 55%). ¹H NMR (400 MHz, DMSO-d₆) δ 12.17 (br, 1H), 11.07 (s, 1H), 7.56 (d, J=8.4 Hz, 1H), 7.17 (t, J=5.6 Hz, 1H), 7.01 (d, J=1.2 Hz, 1H), 6.90 (dd, J=1.6 Hz, 8.4 Hz, 1H), 5.03 (dd, J=5.6 Hz, 12.8 Hz, 1H), 3.61-3.48 (m, 18H), 2.92-2.83 (m, 1H), 2.60-2.54 (m, 2H), 2.43 (t, J=6.4 Hz, 2H), 2.03-1.98 (m, 1H). MS (ESI) m/z=522.1 [M+H]⁺.

### Example 83: 1-((2-(2,6-Dioxopiperidin-3-yl)-1,3-dioxoisoindolin-5-yl)amino)-3,6,9,12,15-pentaoxaoctadecan-18-oic acid (Linker 83)

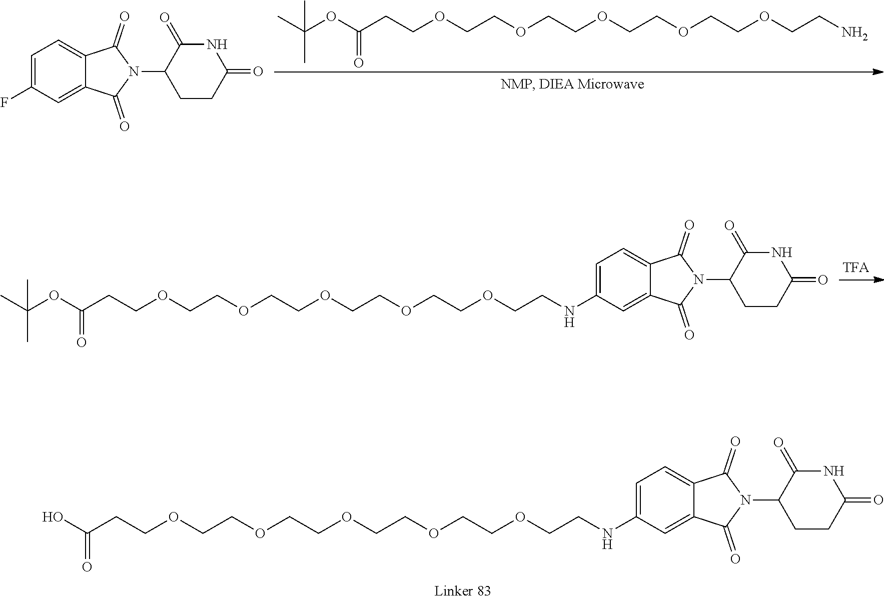 [SMILES: O=C1CCC(N2C(=O)c3ccc(F)cc3C2=O)C(=O)N1 ; CC(C)(C)OC(=O)CCOCCOCCOCCOCCOCCN ; CC(C)(C)OC(=O)CCOCCOCCOCCOCCOCCNc1ccc2c(c1)C(=O)N(C1CCC(=O)NC1=O)C2=O ; O=C(O)CCOCCOCCOCCOCCOCCNc1ccc2c(c1)C(=O)N(C1CCC(=O)NC1=O)C2=O]

Linker 83 was synthesized following the same procedure as Linker 60 as described for Example 60. (1.0 g, yield: 50%). ¹H NMR (400 MHz, DMSO-d₆) δ 12.17 (br, s, 1H), 11.07 (s, 1H), 7.56 (d, J=8.0 Hz, 1H), 7.17 (t, J=5.6 Hz, 1H), 7.01 (s, 1H), 6.90 (dd, J=1.6 Hz, 8.4 Hz, 1H), 5.03 (dd, J=5.6 Hz, 13.2 Hz, 1H), 3.60-3.48 (m, 22H), 2.89-2.83 (m, 1H), 2.60-2.54 (m, 2H), 2.43 (t, J=6.4 Hz, 2H), 2.01-1.98 (m, 1H). MS (ESI) m/z=566.1 [M+H]⁺.

### Example 84. N-(tert-Butyl)-3-((5-methyl-2-((4-(piperazin-1-yl)phenyl)amino)pyrimidin-4-yl)amino)benzenesulfonamide

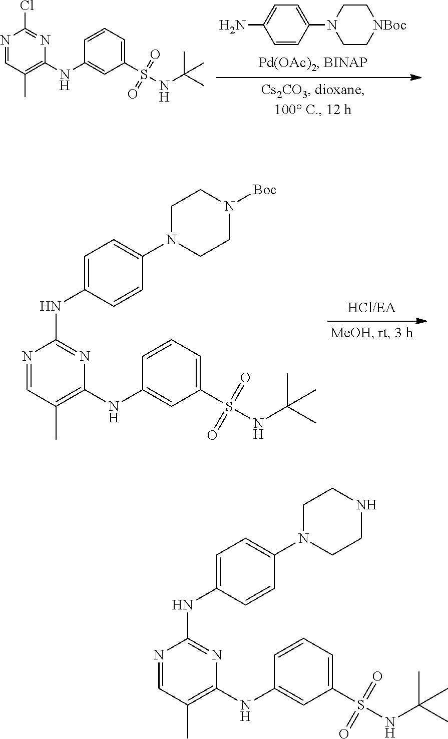 [SMILES: Cc1cnc(Cl)nc1Nc1cccc(S(=O)(=O)NC(C)(C)C)c1 ; CC(C)(C)OC(=O)N1CCN(c2ccc(N)cc2)CC1 ; Cc1cnc(Nc2ccc(N3CCN(C(=O)OC(C)(C)C)CC3)cc2)nc1Nc1cccc(S(=O)(=O)NC(C)(C)C)c1 ; Cc1cnc(Nc2ccc(N3CCNCC3)cc2)nc1Nc1cccc(S(=O)(=O)NC(C)(C)C)c1]

### Step 1: Synthesis of tert-butyl 4-(4-((4-((3-(N-(tert-Butyl)sulfamoyl)phenyl)amino)-5-methylpyrimidin-2-yl)amino)phenyl)piperazine-1-carboxylate

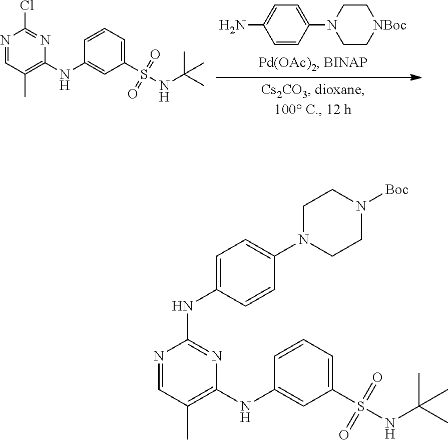 [SMILES: Cc1cnc(Cl)nc1Nc1cccc(S(=O)(=O)NC(C)(C)C)c1 ; CC(C)(C)OC(=O)N1CCN(c2ccc(N)cc2)CC1 ; Cc1cnc(Nc2ccc(N3CCN(C(=O)OC(C)(C)C)CC3)cc2)nc1Nc1cccc(S(=O)(=O)NC(C)(C)C)c1]

To a solution of N-(tert-butyl)-3-((2-chloro-5-methylpyrimidin-4-yl)amino)benzenesulfonamide (1.1 g, 3.1 mmol), tert-butyl 4-(4-aminophenyl)piperazine-1-carboxylate (1.03 g, 3.7 mmol) in 1,4-dioxane (20 mL) was added palladium acetate (70 mg, 0.31 mmol), (+/−)-2,2′-bis(diphenylphosphino)-1,1′-binaphthyl (386 mg, 0.62 mmol), cesium carbonate (2.0 g, 6.2 mmol). The resulting mixture was stirred at 100° C. for 12 h under nitrogen atmosphere. LCMS showed that the reaction was completed. The mixture was diluted with ethyl acetate, washed with water. The organic layer was dried over anhydrous sodium sulphate and concentrated. The residue was purified by column chromatography (hexanes:ethyl acetate=1:1) to give tert-butyl 4-(4-((4-((3-(N-(tert-butyl)sulfamoyl)phenyl)amino)-5-methylpyrimidin-2-yl)amino) phenyl)piperazine-1-carboxylate (1.2 g, yield: 65%) as brown solid. MS (ESI) m/z 596.0 [M+H]⁺.

### Step 2: Synthesis of N-(tert-Butyl)-3-((5-methyl-2-((4-(piperazin-1-yl)phenyl)amino)pyrimidin-4-yl)amino)benzenesulfonamide

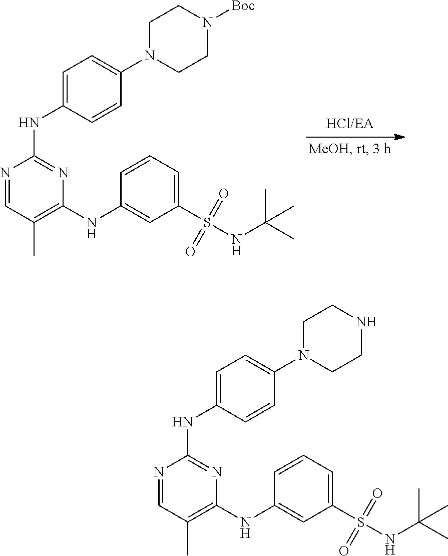 [SMILES: Cc1cnc(Nc2ccc(N3CCN(C(=O)OC(C)(C)C)CC3)cc2)nc1Nc1cccc(S(=O)(=O)NC(C)(C)C)c1 ; Cc1cnc(Nc2ccc(N3CCNCC3)cc2)nc1Nc1cccc(S(=O)(=O)NC(C)(C)C)c1]

To a solution of tert-butyl 4-(4-((4-((3-(N-(tert-butyl)sulfamoyl)phenyl)amino)-5-methylpyrimidin-2-yl)amino)phenyl)piperazine-1-carboxylate (1.2 g, 2.02 mmol) in methanol (250 mL) was added HCl/EtOAc (100 mL, 4 M). The resulting mixture was stirred at room temperature for 3 h. LCMS showed the reaction was completed. The mixture was concentrated, triturated with methanol and ethyl acetate to give N-(tert-butyl)-3-((5-methyl-2-((4-(piperazin-1-yl) phenyl)amino)pyrimidin-4-yl)amino)benzenesulfonamide (1.002 g, yield: 94%) as white solid. ¹H NMR (400 MHz, DMSO-d₆) δ10.59 (s, 1H), 10.02 (s, 1H), 9.50 (s, 2H), 7.96-7.95 (m, 2H), 7.89-7.88 (m, 1H), 7.73 (d, J=8.0 Hz, 1H), 7.64 (s, 1H), 7.58 (t, J=8.0 Hz, 1H), 7.27 (d, J=9.2 Hz, 2H), 6.94 (d, J=8.8 Hz, 2H), 3.36-3.35 (m, 4H), 3.21-3.20 (m, 4H), 2.19 (s, 3H), 1.10 (s, 9H). MS (ESI) m/z 496.2 [M+H]⁺.

### Example 85. 2-(4-(4-((4-((3-(N-(tert-Butyl)sulfamoyl)phenyl)amino)-5-methylpyrimidin-2-yl)amino)phenyl)piperazin-1-yl)acetic acid

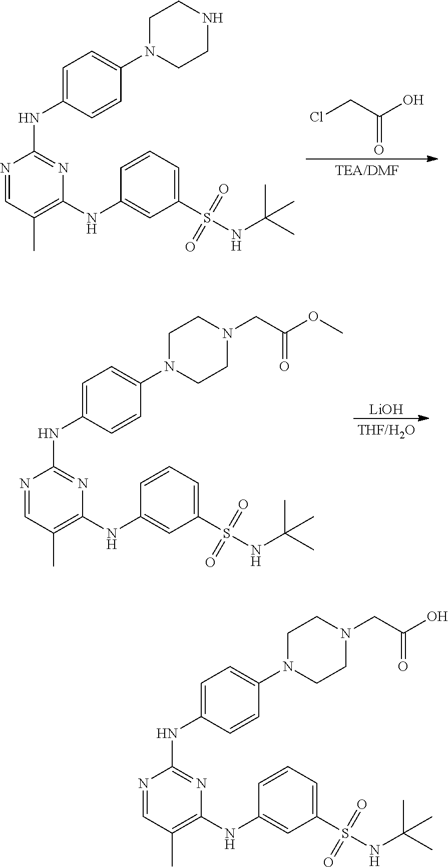 [SMILES: Cc1cnc(Nc2ccc(N3CCNCC3)cc2)nc1Nc1cccc(S(=O)(=O)NC(C)(C)C)c1 ; O=C(O)CCl ; COC(=O)CN1CCN(c2ccc(Nc3ncc(C)c(Nc4cccc(S(=O)(=O)NC(C)(C)C)c4)n3)cc2)CC1 ; "LiOH" ; Cc1cnc(Nc2ccc(N3CCN(CC(=O)O)CC3)cc2)nc1Nc1cccc(S(=O)(=O)NC(C)(C)C)c1]

### Step 1: Synthesis of methyl 2-(4-(4-((4-((3-(N-(tert-butyl)sulfamoyl)phenyl)amino)-5-methylpyrimidin-2-yl)amino)phenyl)piperazin-1-yl)acetate

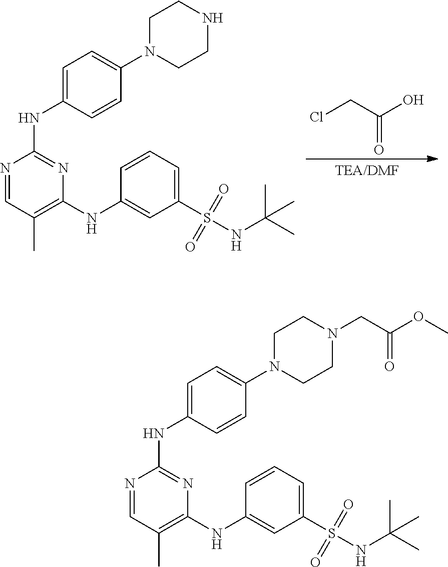 [SMILES: Cc1cnc(Nc2ccc(N3CCNCC3)cc2)nc1Nc1cccc(S(=O)(=O)NC(C)(C)C)c1 ; O=C(O)CCl ; COC(=O)CN1CCN(c2ccc(Nc3ncc(C)c(Nc4cccc(S(=O)(=O)NC(C)(C)C)c4)n3)cc2)CC1]

To a solution of N-tert-butyl-3-[[5-methyl-2-(4-piperazin-1-ylanilino)pyrimidin-4-yl]amino]benzenesulfonamide (275 mg, 0.52 mmol, HCl) in DMF (5 mL) was added Et₃N (208.8 mg, 2.07 mmol) at room temperature. The reaction was stirred at room temperature for 10 min. After addition of methyl 2-chloroacetate (168.26 mg, 1.55 mmol) for over 2 min, the resulting mixture was stirred at room temperature for 2 h, at which time TLC showed the starting material was consumed. The mixture was diluted with H₂O (15 mL), and extracted with EtOAc (10 mL×3). The combined organic layers were dried and concentrated to give 280 mg of crude product which was triturated with 10 mL solvent (petroleum ether:EtOAc=10:1) to get pure methyl 2-[4-[4-[[4-[3-(tert-butylsulfamoyl)anilino]-5-methyl-pyrimidin-2-yl]amino]phenyl]piperazin-1-yl]acetate (240 mg, 82% yield) as white solid which was used directly in the next step.

### Step 2: Synthesis of 2-(4-(4-((4-((3-(N-(tert-butyl)sulfamoyl)phenyl)amino)-5-methylpyrimidin-2-yl)amino)phenyl)piperazin-1-yl)acetic acid

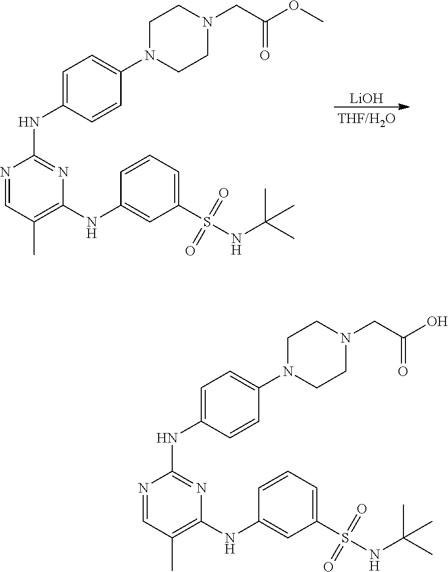 [SMILES: COC(=O)CN1CCN(c2ccc(Nc3ncc(C)c(Nc4cccc(S(=O)(=O)NC(C)(C)C)c4)n3)cc2)CC1 ; "LiOH" ; Cc1cnc(Nc2ccc(N3CCN(CC(=O)O)CC3)cc2)nc1Nc1cccc(S(=O)(=O)NC(C)(C)C)c1]

To a solution of methyl 2-[4-[4-[[4-[3-(tert-butylsulfamoyl)anilino]-5-methyl-pyrimidin-2-yl]amino]phenyl]piperazin-1-yl]acetate (240 mg, 0.42 mmol) in THF (10 mL) was added LiOH (67.64 mg, 1.69 mmol) and H₂O (2 mL) at room temperature. The reaction was stirred for 12 h, at which time TLC showed the starting material was consumed. The mixture was acidified by 0.1 N HCl to pH=6, and concentrated to remove THF. The resulting solid was collected by filtration and dried in vacuum to get 2-[4-[4-[[4-[3-(tert-butylsulfamoyl)anilino]-5-methyl-pyrimidin-2-yl]amino]phenyl]piperazin-1-yl]acetic acid (200 mg, 85% yield) as white solid. MS (ESI) m/z: 554.5 [M−H]⁻.

### Example 86. 5-(4-(Methylsulfonyl)phenyl)-N-(4-(piperazin-1-yl)phenyl)-[1,2,4]triazolo[1,5-a]pyridin-2-amine

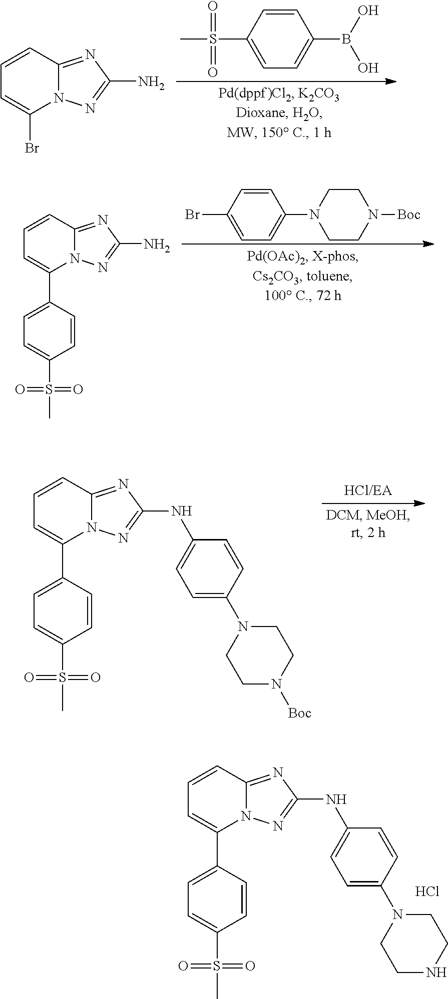

### Step 1: Synthesis of 5-(4-(methylsulfonyl)phenyl)-[1,2,4]triazolo[1,5-a]pyridin-2-amine

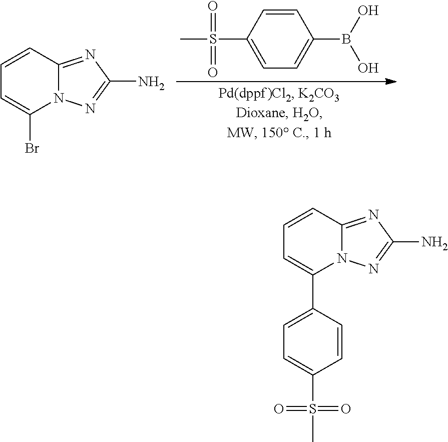 [SMILES: Nc1nc2cccc(Br)n2n1 ; CS(=O)(=O)c1ccc(B(O)O)cc1 ; CS(=O)(=O)c1ccc(-c2cccc3nc(N)nn23)cc1]

A solution of (4-methylsulfonylphenyl)boronic acid (12 g, 60 mmol), 5-bromo-[1,2,4]triazolo[1,5-a]pyridine-2-amine (6.39 g, 30 mmol), 1,1′-bis(diphenylphosphino)ferrocenepalladium(II) chloride (2.2 g, 3 mmol), and potassium carbonate (12.4 g, 90 mmol) in 4:1 dioxane/water (30 mL) was heated at 150° C. under microwave radiation for 1 h. After the reaction mixture was diluted with dichloromethane (100 mL), the resulting suspension was filtered through celite. The filtrate was concentrated, and the resulting residue was purified by flash column chromatography (dichloromethane:methanol=20:1) to provide 5-(4-(methylsulfonyl)phenyl)-[1,2,4]triazolo[1,5-a]pyridin-2-amine (4.0 g, yield: 46%) as white solid. ¹H NMR (400 MHz, DMSO-d₆) δ8.21 (d, J=8.4 Hz, 2H), 8.08 (d, J=8.4 Hz, 2H), 7.58-7.54 (m, 1H), 7.45 (dd, J=8.8 Hz, 0.8 Hz, 1H), 7.13 (dd, J=7.6 Hz, 0.8 Hz, 1H), 6.12 (brs, 2H) 3.32 (s, 3H). MS (ESI) m/z 289.1 [M+H]⁺.

### Step 2: Synthesis of tert-butyl 4-(4-((5-(4-(methylsulfonyl)phenyl)-[1,2,4]triazolo[1,5-a]pyridin-2-yl)amino)phenyl)piperazine-1-carboxylate

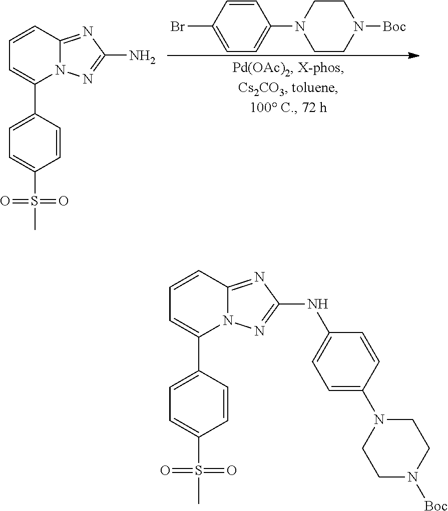 [SMILES: CS(=O)(=O)c1ccc(-c2cccc3nc(N)nn23)cc1 ; CC(C)(C)OC(=O)N1CCN(c2ccc(Br)cc2)CC1 ; CC(C)(C)OC(=O)N1CCN(c2ccc(Nc3nc4cccc(-c5ccc(S(C)(=O)=O)cc5)n4n3)cc2)CC1]

To an oven-dried flask was added 5-(4-methanesulfonyl-phenyl)-[1,2,4]triazolo[1,5-a]-pyridin-2-ylamine (3.79 g, 13.16 mmol), tert-butyl 4-(4-bromophenyl)piperazine-1-carboxylate (4.92 g, 14.48 mmol), palladium acetate (295 mg, 1.32 mmol), 2-(dicyclohexylphosphino)-2′,4′,6′-tri-i-propyl-1,1′-biphenyl(X-phos, 1.25 g, 2.63 mmol), cesium carbonate (8.58 g, 26.32 mmol), and dry toluene (100 mL) under nitrogen. The resulting mixture was vacuumed and purged with nitrogen three times before being heated at 100° C. for 3 d, at which time the reaction mixture was cooled to room temperature and filtered. After the solid was washed with toluene followed by water, it was dried under vacuo and purified by column chromatography (amine dichloromethane:methane=20:1) to give tert-butyl 4-(4-((5-(4-(methylsulfonyl)phenyl)-[1,2,4]triazolo[1,5-a]pyridin-2-yl)amino)phenyl)piperazine-1-carboxylate (3.9 g, yield: 54%) as yellow solid. ¹H NMR (400 MHz, DMSO-d₆) δ9.43 (s, 1H), 8.33 (d, J=8.4 Hz, 2H), 8.14 (d, J=8.4 Hz, 2H), 7.67-7.62 (m, 2H), 7.55 (d, J=8.8 Hz, 2H), 7.27 (dd, J=6.4 Hz, 1.6 Hz, 1H), 6.92 (d, J=9.2 Hz, 2H), 3.46 (t, J=4.8 Hz, 4H), 3.35 (s, 3H), 2.97 (d, J=4.8 Hz, 4H). MS (ESI) m/z 549.1 [M+H]⁺.

### Step 3: Synthesis of 5-(4-(methylsulfonyl)phenyl)-N-(4-(piperazin-1-yl)phenyl)-[1,2,4]triazolo[1,5-a]pyridin-2-amine

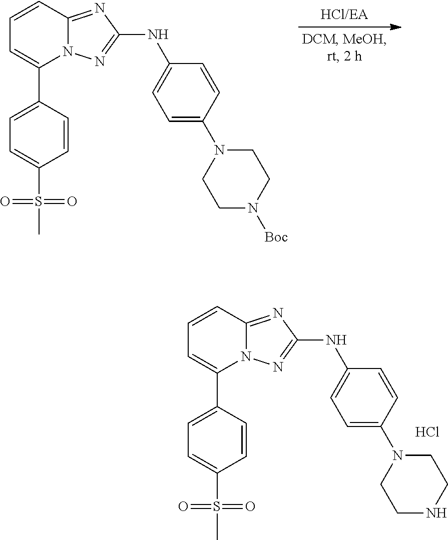 [SMILES: CC(C)(C)OC(=O)N1CCN(c2ccc(Nc3nc4cccc(-c5ccc(S(C)(=O)=O)cc5)n4n3)cc2)CC1 ; CS(=O)(=O)c1ccc(-c2cccc3nc(Nc4ccc(N5CCNCC5)cc4)nn23)cc1 ; Cl]

To a solution tert-butyl 4-(4-((5-(4-(methylsulfonyl)phenyl)-[1,2,4]triazolo[1,5-a]pyridin-2-yl)amino)phenyl) piperazine-1-carboxylate (2.0 g, 3.65 mmol) indichloromethane (50 mL) and methanol (10 mL) was added saturated HCl/EtOAc solution (20 mL, 4 M). After the mixture was stirred at room temperature for 2 h, the precipitate was filtered and dried under vacuo to give 5-(4-(methylsulfonyl)phenyl)-N-(4-(piperazin-1-yl)phenyl)-[1,2,4]triazolo[1,5-a]pyridin-2-amine hydrochloride (1.7 g, yield: 96%) as white solid. ¹H NMR (400 MHz, DMSO-d₆): δ 9.78 (br, 1H), 9.54 (br, 2H), 8.35 (d, J=11.2 Hz, 2H), 8.17 (d, J=11.2 Hz, 2H), 7.78-7.65 (m, 4H), 7.36 (d, J=9.2 Hz, 1H), 7.19 (d, J=8.0 Hz, 1H), 3.46-3.38 (m, 11H). MS (ESI) m/z 449.1 [M+H]⁺.

### Example 87. 2-(4-(4-((5-(4-(Methylsulfonyl)phenyl)-[1,2,4]triazolo[1,5-a]pyridin-2-yl)amino)phenyl)piperazin-1-yl)acetic acid

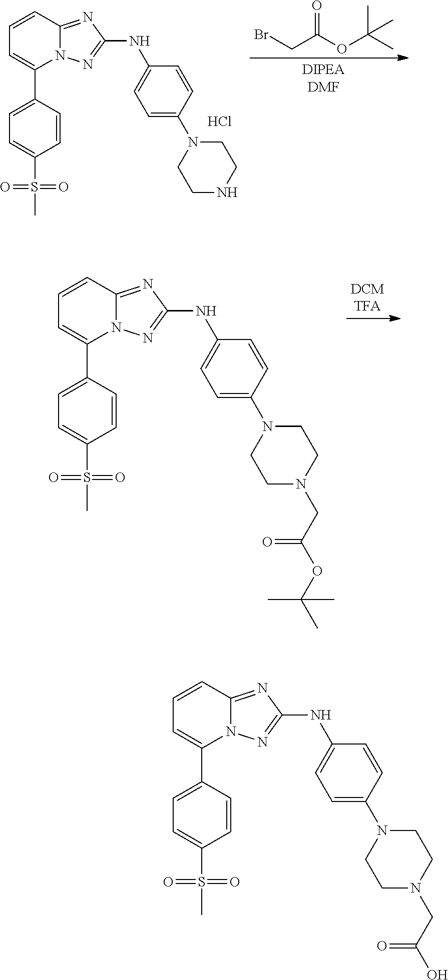

### Step 1: Synthesis of tert-butyl 2-(4-(4-((5-(4-(methylsulfonyl)phenyl)-[1,2,4]triazolo[1,5-a]pyridin-2-yl)amino)phenyl)piperazin-1-yl)acetate

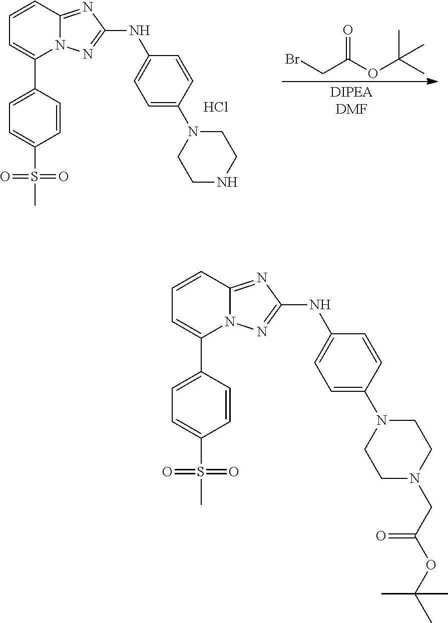

5-(4-(Methylsulfonyl)phenyl)-N-(4-(piperazin-1-yl)phenyl)-[1,2,4]triazolo[1,5-a]pyridin-2-amine (500 mg, 1.11 mmol, HCl salt) in DMF (5 mL) was added DIPEA (289 mg, 2.22 mmol) at room temperature. The reaction was stirred at room temperature for 10 min. After addition of tert-butyl 2-bromoacetate (238 mg, 1.22 mmol) for over 2 min, the resulting mixture was stirred at room temperature for 2 h, at which time TLC showed the starting material was consumed. The mixture was diluted with H₂O (15 mL), and extracted with EtOAc (10 mL×3). The combined organic layers were dried and concentrated to give 430 mg of crude product which was triturated with 10 mL solvent (petroleum ether:EtOAc=10:1) to get pure compound as yellow solid. (430 mg, 70% yield) MS (ESI) m/z: 564.0 [M+H]⁺.

### Step 2: Synthesis of 2-(4-(4-((5-(4-(methylsulfonyl)phenyl)-[1,2,4]triazolo[1,5-a]pyridin-2-yl)amino)phenyl)piperazin-1-yl)acetic acid

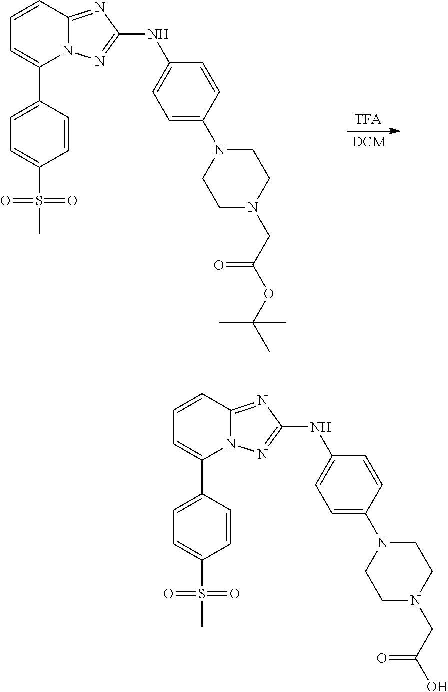 [SMILES: CC(C)(C)OC(=O)CN1CCN(c2ccc(Nc3nc4cccc(-c5ccc(S(C)(=O)=O)cc5)n4n3)cc2)CC1 ; CS(=O)(=O)c1ccc(-c2cccc3nc(Nc4ccc(N5CCN(CC(=O)O)CC5)cc4)nn23)cc1]

To a solution of methyl 2-[4-[4-[[4-[3-(tert-butylsulfamoyl)anilino]-5-methyl-pyrimidin-2-yl]amino]phenyl]piperazin-1-yl]acetate (240 mg, 0.42 mmol) in DCM (10 mL) was added TFA (10 mL) at room temperature. The reaction was stirred for 12 h, at which time TLC showed the starting material was consumed. The mixture was concentrated to remove DCM and TFA. The resulting solid was collected by filtration and dried in vacuum to afford 2-(4-(4-((5-(4-(methylsulfonyl)phenyl)-[1,2,4]triazolo[1,5-a]pyridin-2-yl)amino)phenyl)piperazin-1-yl)acetic acid (183 mg, 85% yield) as yellow solid. MS (ESI) m/z: 507.1 [M+H]⁺.

### Example 88

### 2-(4-(4-(8-(3,5-Difluoro-4-(morpholinomethyl)phenyl)quinoxalin-2-yl)-1H-pyrazol-1-yl)piperidin-1-yl)acetic acid

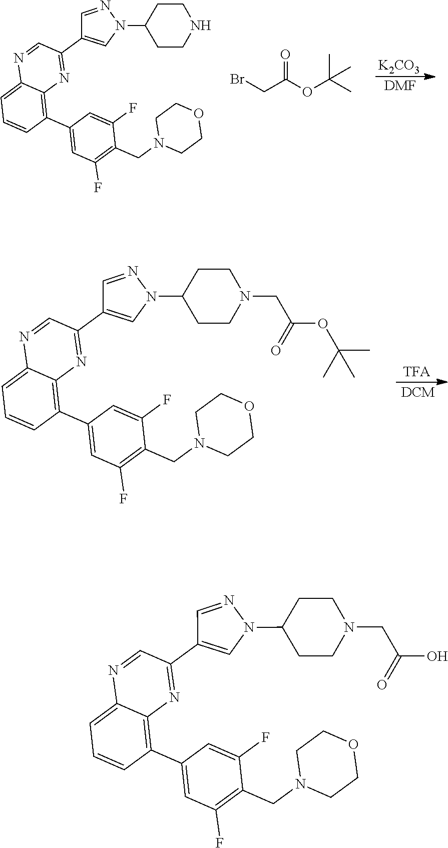

### Step 1: Synthesis of tert-butyl 2-(4-(4-(8-(3,5-difluoro-4-(morpholinomethyl)phenyl) quinoxalin-2-yl)-1H-pyrazol-1-yl)piperidin-1-yl)acetate

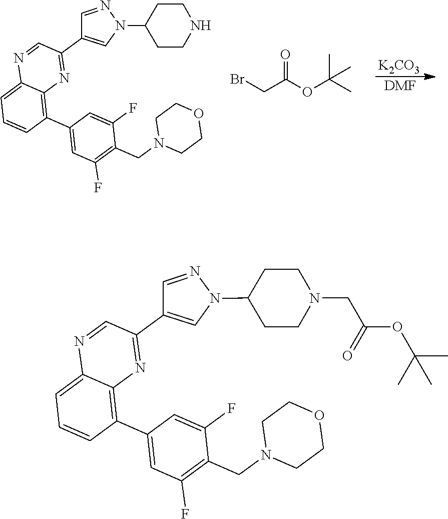

To a solution of 4-[[2,6-difluoro-4-[3-[1-(4-piperidyl)pyrazol-4-yl]quinoxalin-5-yl]phenyl]methyl]morpholine (109.37 mg, 211.80 umol) in DMF (3 mL) was added K₂CO₃ (73.18 mg, 529.50 umol), followed by tert-Butyl 2-bromoacetate (47.84 mg, 232.98 umol) over 5 min. The resulting mixture was stirred at 25° C. for 3 h. After the amine was totally consumed, the reaction was poured into water (300 mL) and extracted with ethyl acetate (50 mL×3). The combined organic layers were washed with saturated brine (100 mL), dried over anhydrous sodium sulfate, filtered and evaporated under reduced pressure. The resulting residue was purified by silica gel column chromatography to afford the desired product tert-butyl 2-[4-[4-[8-[3,5-difluoro-4-(morpholinomethyl)phenyl]quinoxalin-2-yl]pyrazol-1-yl]-1-piperidyl]acetate (112 mg, 87.4% yield) as light yellow solid. MS (ESI) m/z: 605.3 [M+H]⁺.

### Step 2: Synthesis of 2-(4-(4-(8-(3,5-difluoro-4-(morpholinomethyl)phenyl)quinoxalin-2-yl)-1H-pyrazol-1-yl)piperidin-1-yl)acetic acid

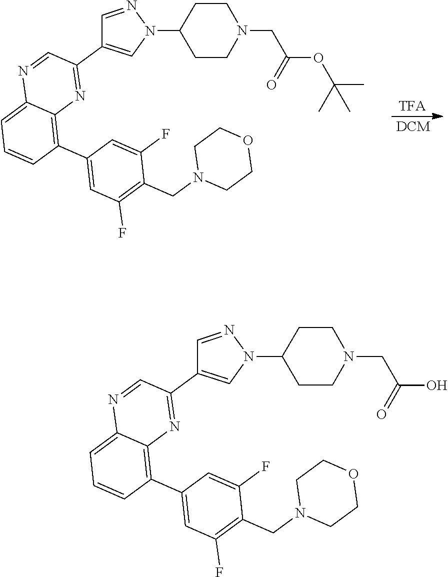 [SMILES: CC(C)(C)OC(=O)CN1CCC(n2cc(-c3cnc4cccc(-c5cc(F)c(CN6CCOCC6)c(F)c5)c4n3)cn2)CC1 ; O=C(O)CN1CCC(n2cc(-c3cnc4cccc(-c5cc(F)c(CN6CCOCC6)c(F)c5)c4n3)cn2)CC1]

To a solution of tert-butyl 2-[4-[4-[8-[3,5-difluoro-4-(morpholinomethyl)phenyl]quinoxalin-2-yl]pyrazol-1-yl]-1-piperidyl]acetate (110 mg, 181.91 umol) in DCM (2 mL) was added TFA (8 g, 70.16 mmol). The resulting mixture was stirred at 25° C. for 3 h. After the starting material was totally consumed, the reaction was evaporated under reduced pressure. The resulting residue was purified by reverse-phase chromatography to yield the desired product 2-[4-[4-[8-[3,5-difluoro-4-(morpholino methyl)phenyl]quinoxalin-2-yl]pyrazol-1-yl]-1-piperidyl]acetic acid (86 mg, 86.2% yield) as light yellow solid. MS (ESI) m/z: 547.2 [M−H]⁻.

### Example 89. 4-[[6-[4-[4-[8-[3,5-Difluoro-4-(morpholinomethyl)phenyl]quinoxalin-2-yl]pyrazol-1-yl]-1-piperidyl]-6-oxo-hexyl]amino]-2-(2,6-dioxo-3-piperidyl)isoindoline-1,3-dione JA-001

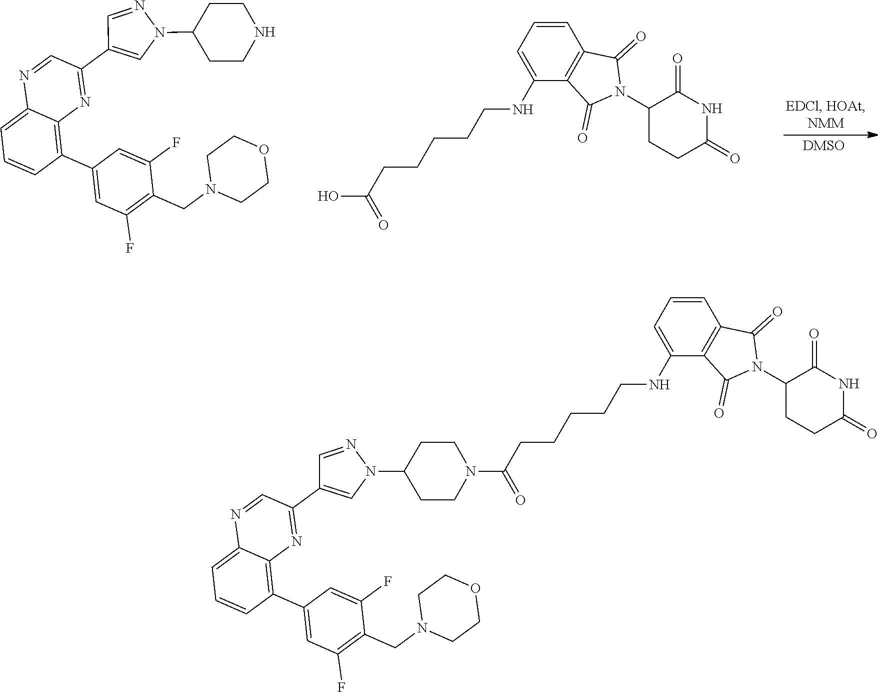 [SMILES: Fc1cc(-c2cccc3ncc(-c4cnn(C5CCNCC5)c4)nc23)cc(F)c1CN1CCOCC1 ; O=C(O)CCCCCNc1cccc2c1C(=O)N(C1CCC(=O)NC1=O)C2=O ; O=C1CCC(N2C(=O)c3cccc(NCCCCCC(=O)N4CCC(n5cc(-c6cnc7cccc(-c8cc(F)c(CN9CCOCC9)c(F)c8)c7n6)cn5)CC4)c3C2=O)C(=O)N1]

To a solution of 6-[[2-(2,6-dioxo-3-piperidyl)-1,3-dioxo-isoindolin-4-yl]amino]hexanoic acid (6.32 mg, 16.31 umol) in DMSO (1 mL) was added HOAt (2.22 mg, 16.31 umol), EDCI (3.13 mg, 16.31 umol), NMM (8.25 mg, 81.54 umol) and 4-[[2,6-difluoro-4-[3-[1-(4-piperidyl)pyrazol-4-yl]quinoxalin-5-yl]phenyl]methyl]morpholine (8 mg, 16.31 umol) sequentially. The resulting solution was stirred at 25° C. for 16 h, before the reaction was poured into water (200 mL) and extracted with ethyl acetate (50 mL×3). The combined organic layers were washed with saturated brine (100 mL), dried over anhydrous sodium sulfate, filtered and evaporated under reduced pressure. The resulting residue was purified by reverse-phase chromatography to yield the desired product 4-[[6-[4-[4-[8-[3,5-difluoro-4-(morpholinomethyl)phenyl]quinoxalin-2-yl]pyrazol-1-yl]-1-piperidyl]-6-oxo-hexyl]amino]-2-(2,6-dioxo-3-piperidyl)isoindoline-1,3-dione (6.3 mg, 44.9% yield) as light yellow solid. MS (ESI) m/z: 860.4 [M+H]⁺.

### Example 90. 4-[[2-[4-[4-[8-[3,5-Difluoro-4-(morpholinomethyl)phenyl]quinoxalin-2-yl]pyrazol-1-yl]-1-piperidyl]-2-oxo-ethyl]amino]-2-(2,6-dioxo-3-piperidyl)isoindoline-1,3-dione (JA-002)

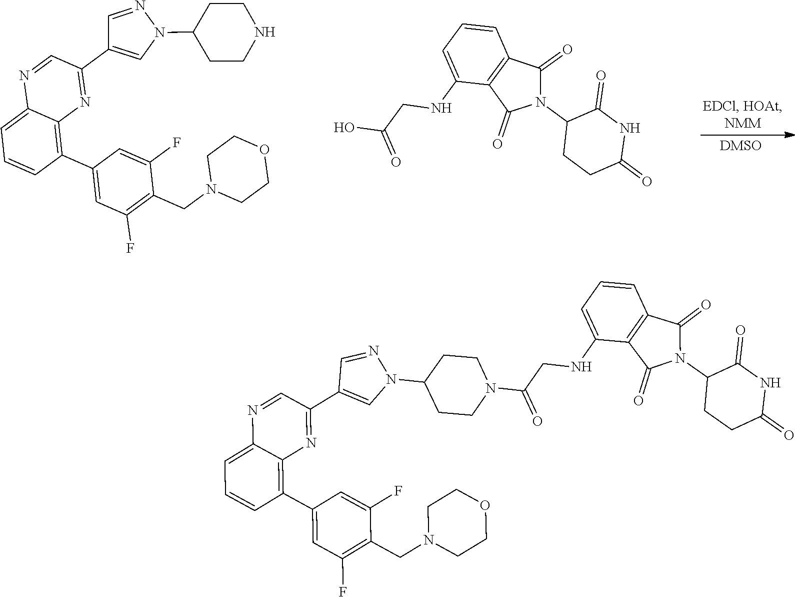 [SMILES: Fc1cc(-c2cccc3ncc(-c4cnn(C5CCNCC5)c4)nc23)cc(F)c1CN1CCOCC1 ; O=C(O)CNc1cccc2c1C(=O)N(C1CCC(=O)NC1=O)C2=O ; O=C1CCC(N2C(=O)c3cccc(NCC(=O)N4CCC(n5cc(-c6cnc7cccc(-c8cc(F)c(CN9CCOCC9)c(F)c8)c7n6)cn5)CC4)c3C2=O)C(=O)N1]

JA-002 was synthesized following the standard procedure for preparing JA-001 (6.9 mg, 52.6% yield). MS (ESI) m/z: 804.3 [M+H]⁺.

### Example 91. 4-[[5-[4-[4-[8-[3,5-Difluoro-4-(morpholinomethyl)phenyl]quinoxalin-2-yl]pyrazol-1-yl]-1-piperidyl]-5-oxo-pentyl]amino]-2-(2,6-dioxo-3-piperidyl)isoindoline-1,3-dione (JA-003)

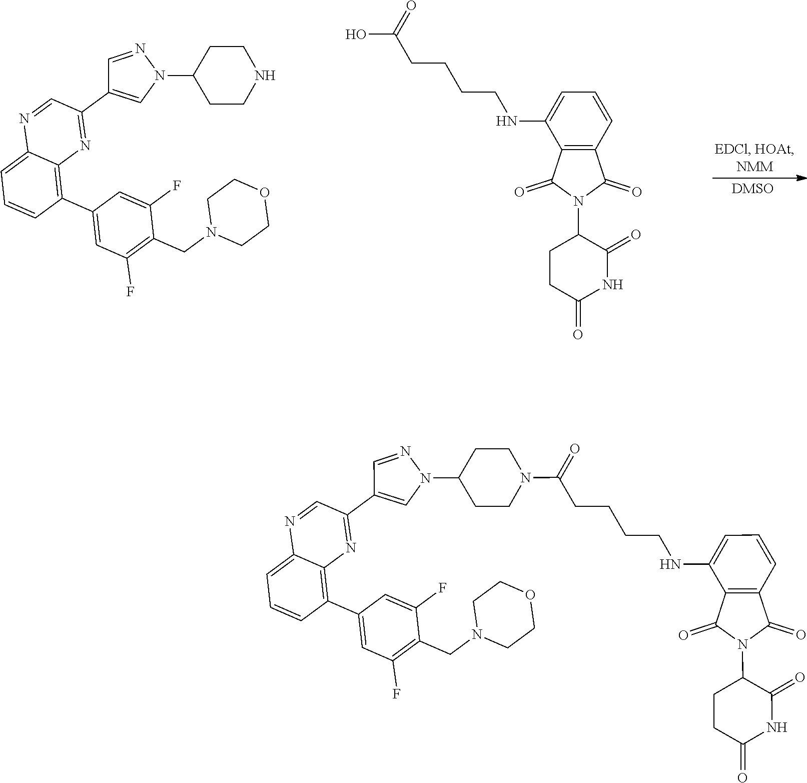 [SMILES: Fc1cc(-c2cccc3ncc(-c4cnn(C5CCNCC5)c4)nc23)cc(F)c1CN1CCOCC1 ; O=C(O)CCCCNc1cccc2c1C(=O)N(C1CCC(=O)NC1=O)C2=O ; O=C1CCC(N2C(=O)c3cccc(NCCCCC(=O)N4CCC(n5cc(-c6cnc7cccc(-c8cc(F)c(CN9CCOCC9)c(F)c8)c7n6)cn5)CC4)c3C2=O)C(=O)N1]

JA-003 was synthesized following the standard procedure for preparing JA-001 (7.6 mg, 55.1% yield). MS (ESI) m/z: 846.3 [M+H]⁺.

### Example 92. 4-[[3-[4-[4-[8-[3,5-Difluoro-4-(morpholinomethyl)phenyl]quinoxalin-2-yl]pyrazol-1-yl]-1-piperidyl]-3-oxo-propyl]amino]-2-(2,6-dioxo-3-piperidyl)isoindoline-1,3-dione (JA-004)

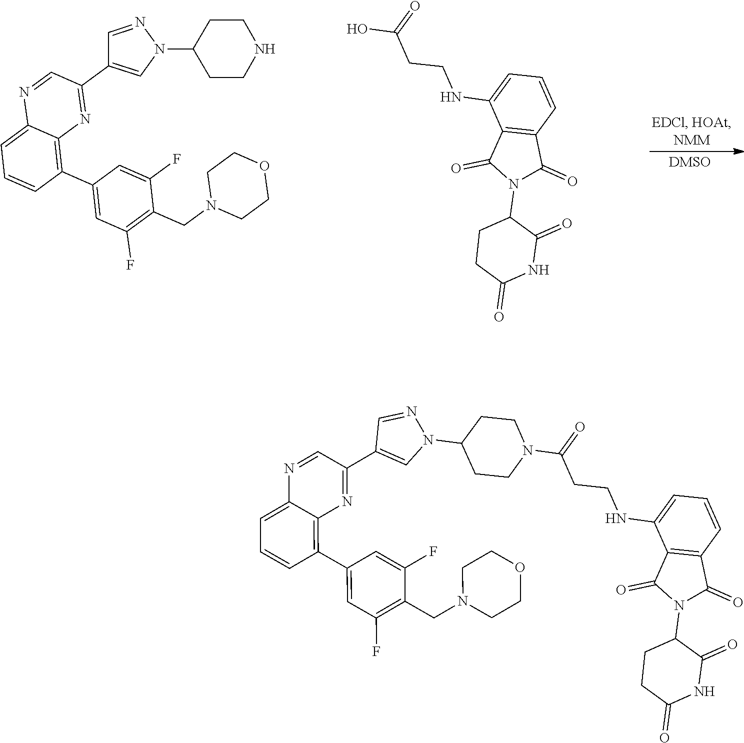 [SMILES: Fc1cc(-c2cccc3ncc(-c4cnn(C5CCNCC5)c4)nc23)cc(F)c1CN1CCOCC1 ; O=C(O)CCNc1cccc2c1C(=O)N(C1CCC(=O)NC1=O)C2=O ; O=C1CCC(N2C(=O)c3cccc(NCCC(=O)N4CCC(n5cc(-c6cnc7cccc(-c8cc(F)c(CN9CCOCC9)c(F)c8)c7n6)cn5)CC4)c3C2=O)C(=O)N1]

JA-004 was synthesized following the standard procedure for preparing JA-001 (7.9 mg, 59.2% yield). MS (ESI) m/z: 818.3 [M+H]⁺.

### Example 93. 4-[[4-[4-[4-[8-[3,5-Difluoro-4-(morpholinomethyl)phenyl]quinoxalin-2-yl]pyrazol-1-yl]-1-piperidyl]-4-oxo-butyl]amino]-2-(2,6-dioxo-3-piperidyl)isoindoline-1,3-dione (JA-005)

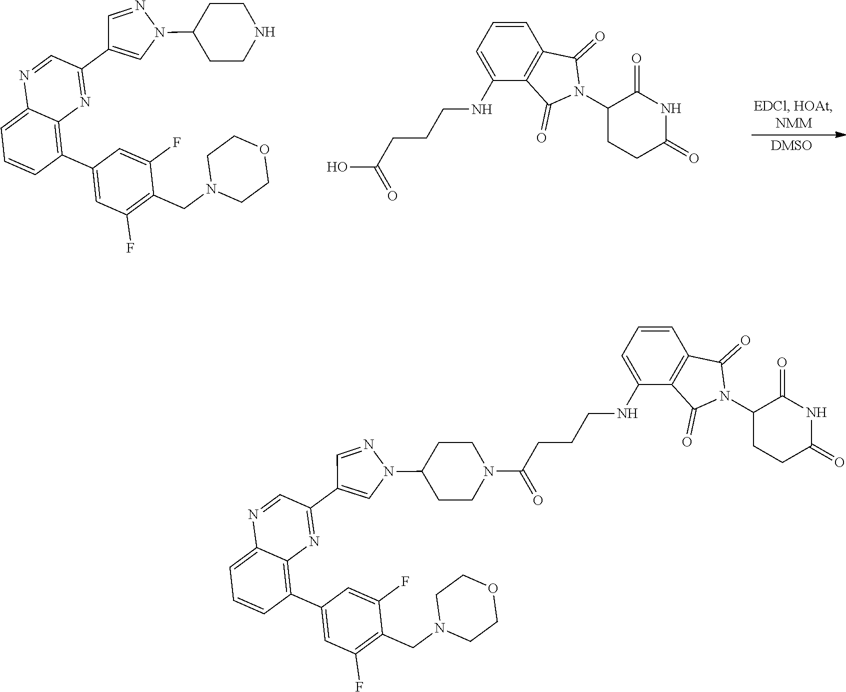 [SMILES: Fc1cc(-c2cccc3ncc(-c4cnn(C5CCNCC5)c4)nc23)cc(F)c1CN1CCOCC1 ; O=C(O)CCCNc1cccc2c1C(=O)N(C1CCC(=O)NC1=O)C2=O ; O=C1CCC(N2C(=O)c3cccc(NCCCC(=O)N4CCC(n5cc(-c6cnc7cccc(-c8cc(F)c(CN9CCOCC9)c(F)c8)c7n6)cn5)CC4)c3C2=O)C(=O)N1]

JA-005 was synthesized following the standard procedure for preparing JA-001 (8.1 mg, 59.7% yield). MS (ESI) m/z: 832.3 [M+H]⁺.

### Example 94. N-(tert-Butyl)-3-((2-((4-(4-((2-(2,6-dioxopiperidin-3-yl)-1,3-dioxoisoindolin-4-yl)glycyl)piperazin-1-yl)phenyl)amino)-5-methylpyrimidin-4-yl)amino)benzenesulfonamide (JA-006)

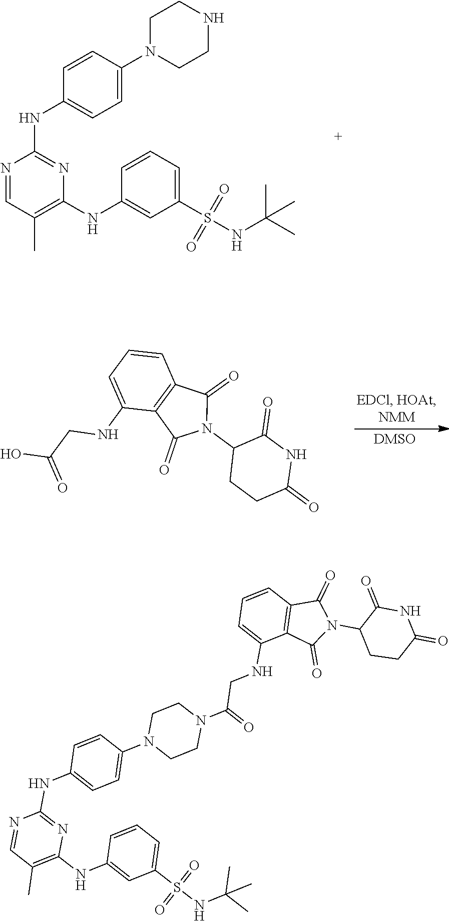 [SMILES: Cc1cnc(Nc2ccc(N3CCNCC3)cc2)nc1Nc1cccc(S(=O)(=O)NC(C)(C)C)c1 ; O=C(O)CNc1cccc2c1C(=O)N(C1CCC(=O)NC1=O)C2=O ; Cc1cnc(Nc2ccc(N3CCN(C(=O)CNc4cccc5c4C(=O)N(C4CCC(=O)NC4=O)C5=O)CC3)cc2)nc1Nc1cccc(S(=O)(=O)NC(C)(C)C)c1]

JA-006 was synthesized following the standard procedure for preparing JA-015 (7.7 mg, 55.1% yield). MS (ESI) m/z: 809.3 [M+H]⁺.

### Example 95. N-(tert-Butyl)-3-((2-((4-(4-(6-((2-(2,6-dioxopiperidin-3-yl)-1,3-dioxoisoindolin-4-yl)amino)hexanoyl)piperazin-1-yl)phenyl)amino)-5-methylpyrimidin-4-yl)amino)benzenesulfonamide (JA-007)

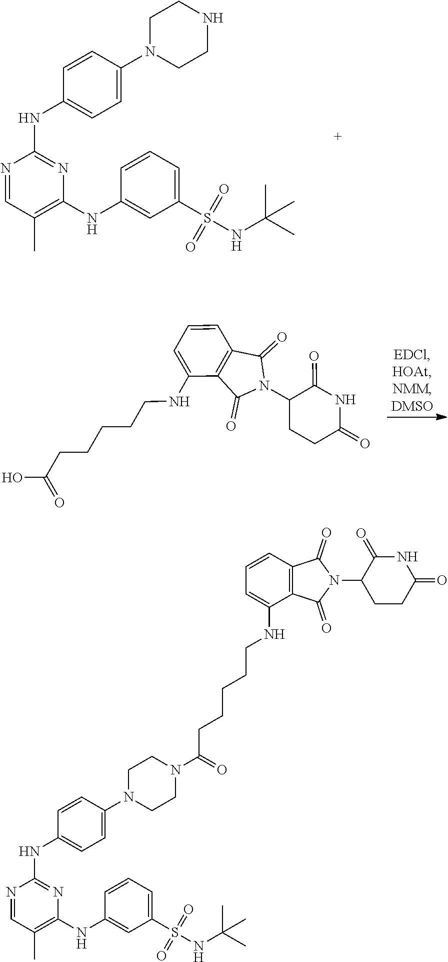 [SMILES: Cc1cnc(Nc2ccc(N3CCNCC3)cc2)nc1Nc1cccc(S(=O)(=O)NC(C)(C)C)c1 ; O=C(O)CCCCCNc1cccc2c1C(=O)N(C1CCC(=O)NC1=O)C2=O ; Cc1cnc(Nc2ccc(N3CCN(C(=O)CCCCCNc4cccc5c4C(=O)N(C4CCC(=O)NC4=O)C5=O)CC3)cc2)nc1Nc1cccc(S(=O)(=O)NC(C)(C)C)c1]

JA-007 was synthesized following the standard procedure for preparing JA-015 (7.1 mg, 53.6% yield). MS (ESI) m/z: 865.4 [M+H]⁺.

### Example 96. N-(tert-Butyl)-3-((2-((4-(4-(4-((2-(2,6-dioxopiperidin-3-yl)-1,3-dioxoisoindolin-4-yl)amino)butanoyl)piperazin-1-yl)phenyl)amino)-5-methylpyrimidin-4-yl)amino)benzenesulfonamide (JA-008)

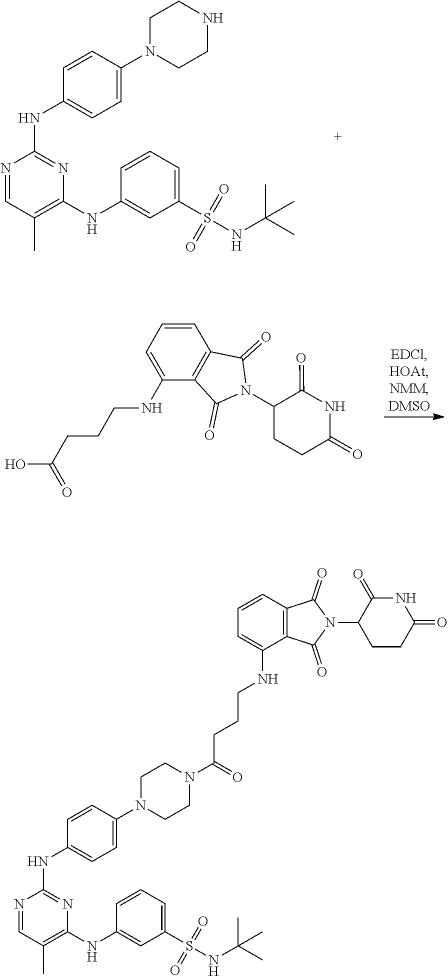 [SMILES: Cc1cnc(Nc2ccc(N3CCNCC3)cc2)nc1Nc1cccc(S(=O)(=O)NC(C)(C)C)c1 ; O=C(O)CCCNc1cccc2c1C(=O)N(C1CCC(=O)NC1=O)C2=O ; Cc1cnc(Nc2ccc(N3CCN(C(=O)CCCNc4cccc5c4C(=O)N(C4CCC(=O)NC4=O)C5=O)CC3)cc2)nc1Nc1cccc(S(=O)(=O)NC(C)(C)C)c1]

JA-008 was synthesized following the standard procedure for preparing JA-015 (7.4 mg, 54.5% yield). MS (ESI) m/z: 837.3 [M+H]⁺.

### Example 97. N-(tert-Butyl)-3-((2-((4-(4-(3-((2-(2,6-dioxopiperidin-3-yl)-1,3-dioxoisoindolin-4-yl)amino)propanoyl)piperazin-1-yl)phenyl)amino)-5-methylpyrimidin-4-yl)amino)benzenesulfonamide (JA-009)

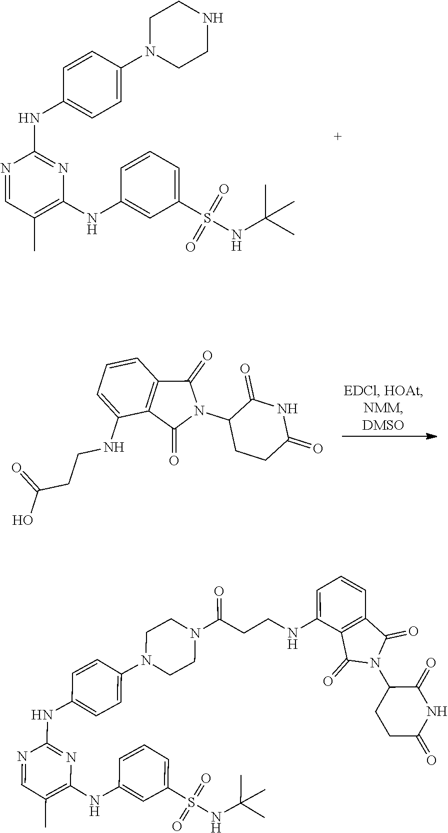 [SMILES: Cc1cnc(Nc2ccc(N3CCNCC3)cc2)nc1Nc1cccc(S(=O)(=O)NC(C)(C)C)c1 ; O=C(O)CCNc1cccc2c1C(=O)N(C1CCC(=O)NC1=O)C2=O ; Cc1cnc(Nc2ccc(N3CCN(C(=O)CCNc4cccc5c4C(=O)N(C4CCC(=O)NC4=O)C5=O)CC3)cc2)nc1Nc1cccc(S(=O)(=O)NC(C)(C)C)c1]

JA-009 was synthesized following the standard procedure for preparing JA-015 (7.5 mg, 56.2% yield). MS (ESI) m/z: 823.3 [M+H]⁺.

### Example 98. 2-(2,6-Dioxopiperidin-3-yl)-4-((4-(4-(4-((5-(4-(methylsulfonyl)phenyl)-[1,2,4]triazolo[1,5-a]pyridin-2-yl)amino)phenyl)piperazin-1-yl)-4-oxobutyl)amino)isoindoline-1,3-dione (JA-010)

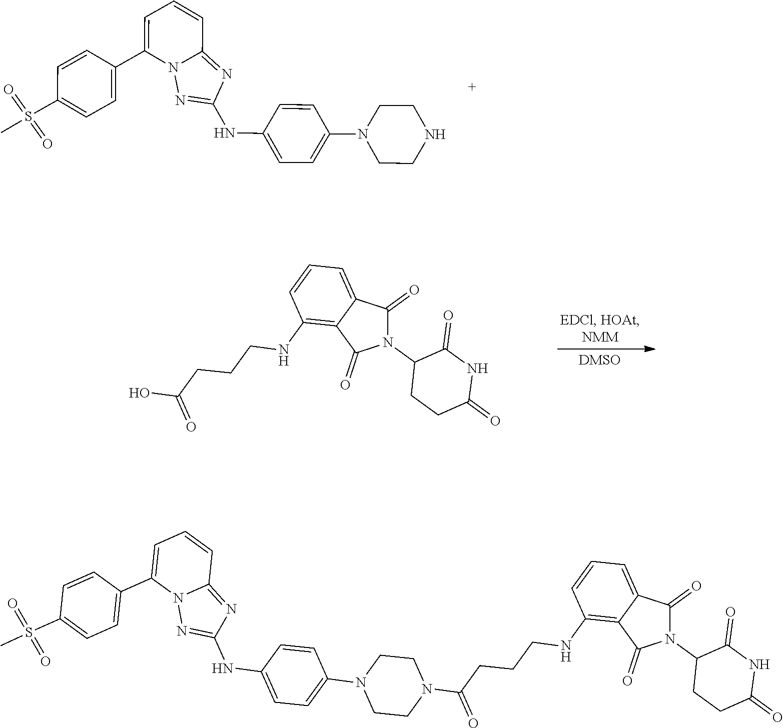 [SMILES: CS(=O)(=O)c1ccc(-c2cccc3nc(Nc4ccc(N5CCNCC5)cc4)nn23)cc1 ; O=C(O)CCCNc1cccc2c1C(=O)N(C1CCC(=O)NC1=O)C2=O ; CS(=O)(=O)c1ccc(-c2cccc3nc(Nc4ccc(N5CCN(C(=O)CCCNc6cccc7c6C(=O)N(C6CCC(=O)NC6=O)C7=O)CC5)cc4)nn23)cc1]

To a solution of 4-((2-(2,6-dioxopiperidin-3-yl)-1,3-dioxoisoindolin-4-yl)amino)butanoic acid (7.90 mg, 0.022 mmol) was added HOAt (2.72 mg, 0.020 mmol), EDCI (3.87 mg, 0.020 mmol) and NMM (2.04 mg, 0.020 mmol). The resulting mixture was stirred at room temperature for 2 min, before [5-(4-methanesulfonyl-phenyl)-[1,2,4]triazolo[1,5-a]pyridin-2-yl]-(4-piperazin-1-yl-phenyl)-amine (8.9 mg, 0.020 mmol) was added. After the reaction was stirred at 25° C. for 12 h, TLC showed the reaction was completed. The mixture was purified by reverse phase flash chromatography, followed by prep-TLC to give 2-(2,6-dioxopiperidin-3-yl)-4-((4-(4-(4-((5-(4-(methylsulfonyl)phenyl)-[1,2,4]triazolo[1,5-a]pyridin-2-yl)amino)phenyl)piperazin-1-yl)-4-oxobutyl)amino)isoindoline-1,3-dione (11.9 mg, 76% yield). MS (ESI) m/z: 790.6 [M+H]⁺.

### Example 99. 2-(2,6-Dioxo-piperidin-3-yl)-4-[5-(4-{4-[5-(4-methanesulfonyl-phenyl)-[1,2,4]triazolo[1,5-a]pyridin-2-ylamino]-phenyl}-piperazin-1-yl)-5-oxo-pentylamino]-isoindole-1,3-dione (JA-011)

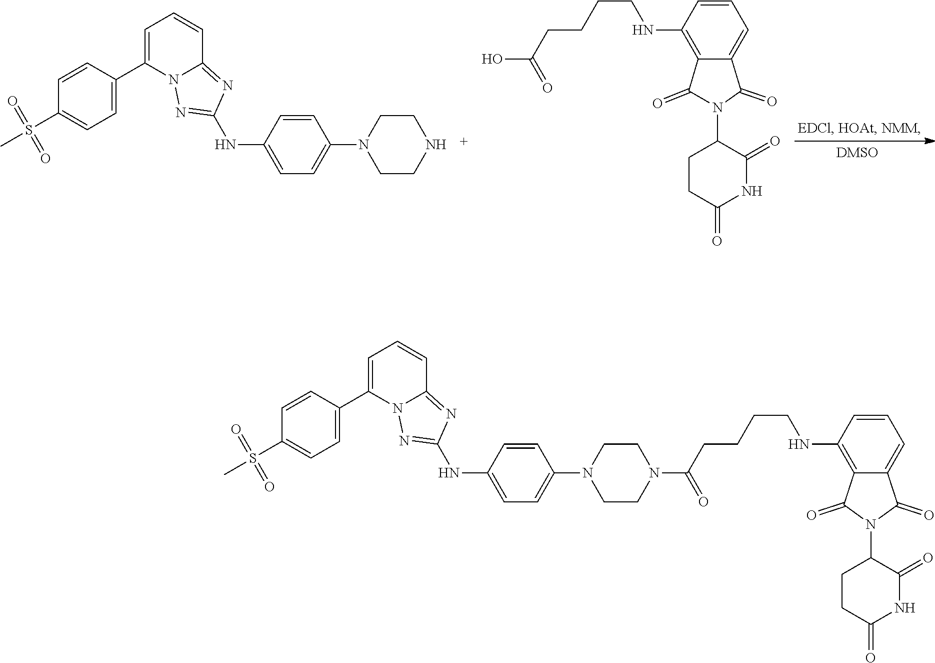 [SMILES: CS(=O)(=O)c1ccc(-c2cccc3nc(Nc4ccc(N5CCNCC5)cc4)nn23)cc1 ; O=C(O)CCCCNc1cccc2c1C(=O)N(C1CCC(=O)NC1=O)C2=O ; CS(=O)(=O)c1ccc(-c2cccc3nc(Nc4ccc(N5CCN(C(=O)CCCCNc6cccc7c6C(=O)N(C6CCC(=O)NC6=O)C7=O)CC5)cc4)nn23)cc1]

JA-011 was synthesized following the standard procedure for preparing JA-010 (10 mg, 77% yield). MS (ESI) m/z: 804.37 [M+H]⁺.

### Example 100. 2-(2,6-Dioxo-piperidin-3-yl)-4-[3-(4-{4-[5-(4-methanesulfonyl-phenyl)-[1,2,4]triazolo[1,5-a]pyridin-2-ylamino]-phenyl}-piperazin-1-yl)-3-oxo-propylamino]-isoindole-1,3-dione (JA-012)

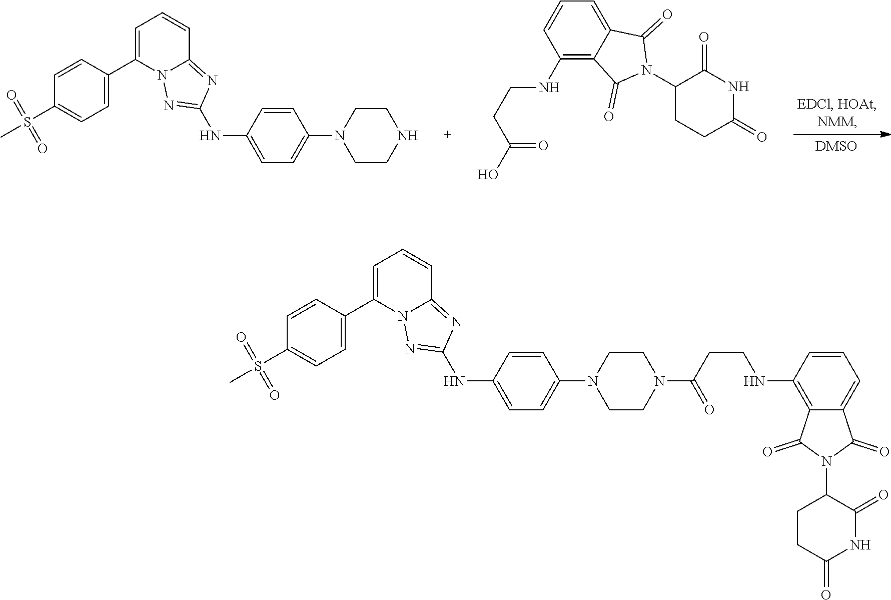 [SMILES: CS(=O)(=O)c1ccc(-c2cccc3nc(Nc4ccc(N5CCNCC5)cc4)nn23)cc1 ; O=C(O)CCNc1cccc2c1C(=O)N(C1CCC(=O)NC1=O)C2=O ; CS(=O)(=O)c1ccc(-c2cccc3nc(Nc4ccc(N5CCN(C(=O)CCNc6cccc7c6C(=O)N(C6CCC(=O)NC6=O)C7=O)CC5)cc4)nn23)cc1]

JA-012 was synthesized following the standard procedure for preparing JA-010 (9 mg, 75% yield). MS (ESI) m/z: 776.3 [M+H]⁺.

### Example 101. 2-(2,6-Dioxo-piperidin-3-yl)-4-[6-(4-{4-[5-(4-methanesulfonyl-phenyl)-[1,2,4]triazolo[1,5-a]pyridin-2-ylamino]-phenyl}-piperazin-1-yl)-6-oxo-hexylamino]-isoindole-1,3-dione (JA-013)

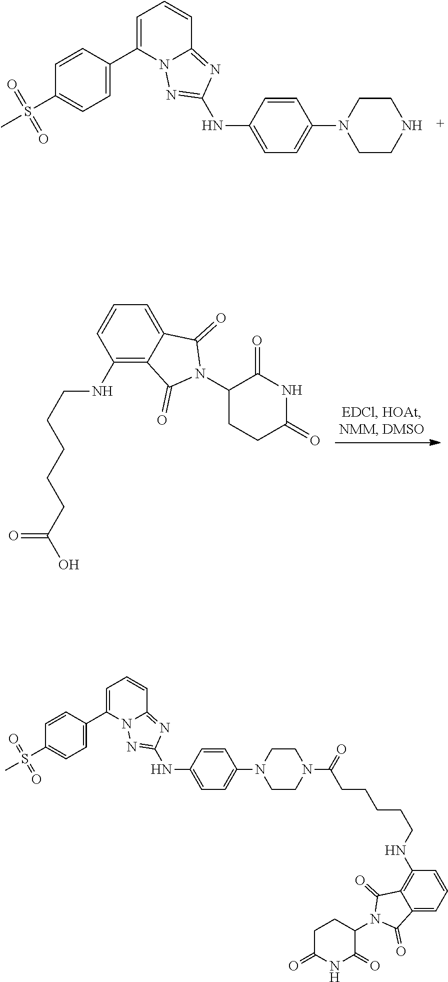 [SMILES: CS(=O)(=O)c1ccc(-c2cccc3nc(Nc4ccc(N5CCNCC5)cc4)nn23)cc1 ; O=C(O)CCCCCNc1cccc2c1C(=O)N(C1CCC(=O)NC1=O)C2=O ; CS(=O)(=O)c1ccc(-c2cccc3nc(Nc4ccc(N5CCN(C(=O)CCCCCNc6cccc7c6C(=O)N(C6CCC(=O)NC6=O)C7=O)CC5)cc4)nn23)cc1]

JA-013 was synthesized following the standard procedure for preparing JA-010 (11 mg, 77% yield). MS (ESI) m/z: 817.3 [M+H]⁺.

### Example 102. 2-(2,6-Dioxo-piperidin-3-yl)-4-[2-(4-{4-[5-(4-methanesulfonyl-phenyl)-[1,2,4]triazolo[1,5-a]pyridin-2-ylamino]-phenyl}-piperazin-1-yl)-2-oxo-ethylamino]-isoindole-1,3-dione (JA-014)

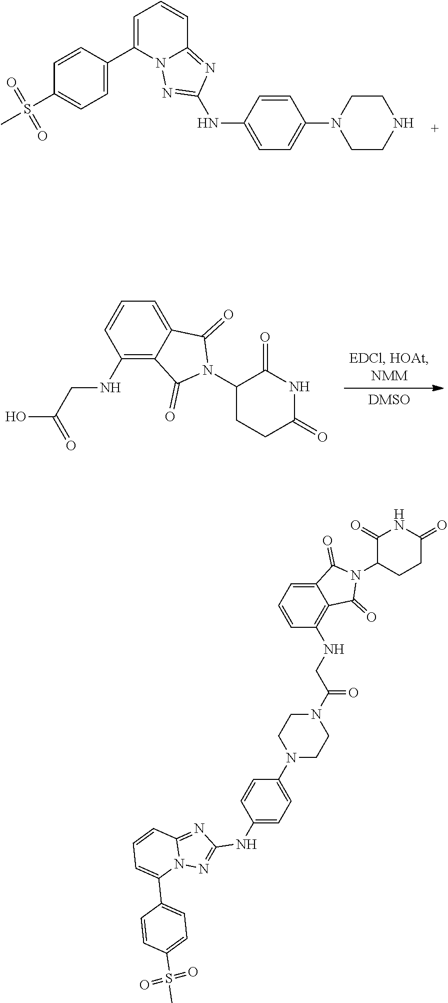 [SMILES: CS(=O)(=O)c1ccc(-c2cccc3nc(Nc4ccc(N5CCNCC5)cc4)nn23)cc1 ; O=C(O)CNc1cccc2c1C(=O)N(C1CCC(=O)NC1=O)C2=O ; CS(=O)(=O)c1ccc(-c2cccc3nc(Nc4ccc(N5CCN(C(=O)CNc6cccc7c6C(=O)N(C6CCC(=O)NC6=O)C7=O)CC5)cc4)nn23)cc1]

JA-014 was synthesized following the standard procedure for preparing JA-010 (8 mg, 76% yield). MS (ESI) m/z: 762.2 [M+H]⁺.

### Example 103. N-(tert-Butyl)-3-((2-((4-(4-(7-((2-(2,6-dioxopiperidin-3-yl)-1,3-dioxoisoindolin-4-yl)amino)heptanoyl)piperazin-1-yl)phenyl)amino)-5-methylpyrimidin-4-yl)amino)benzenesulfonamide (JA-015)

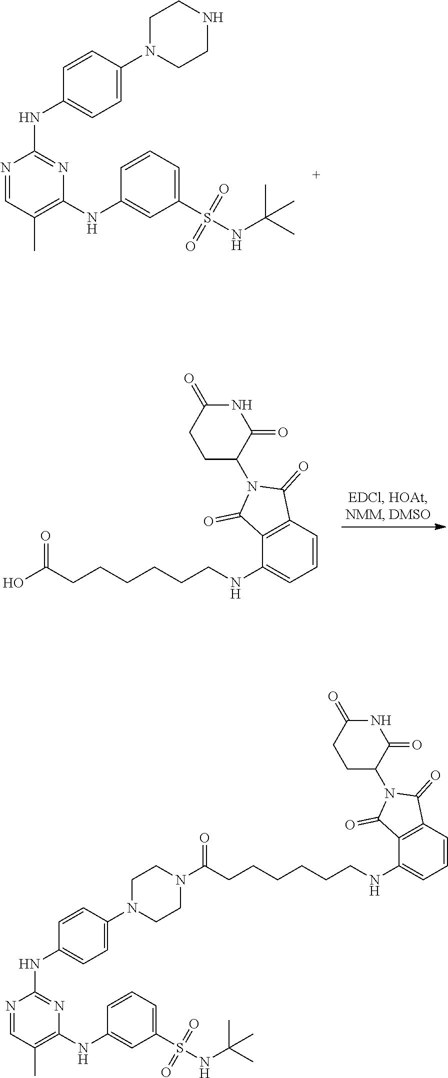 [SMILES: Cc1cnc(Nc2ccc(N3CCNCC3)cc2)nc1Nc1cccc(S(=O)(=O)NC(C)(C)C)c1 ; O=C(O)CCCCCCNc1cccc2c1C(=O)N(C1CCC(=O)NC1=O)C2=O ; Cc1cnc(Nc2ccc(N3CCN(C(=O)CCCCCCNc4cccc5c4C(=O)N(C4CCC(=O)NC4=O)C5=O)CC3)cc2)nc1Nc1cccc(S(=O)(=O)NC(C)(C)C)c1]

To a solution of 7-[[2-(2,6-dioxo-3-piperidyl)-1,3-dioxo-isoindolin-4-yl]amino]heptanoic acid (8.91 mg, 0.022 mmol) was added HOAt (2.72 mg, 0.020 mmol), EDCI (3.87 mg, 0.020 mmol) and NMM (2.04 mg, 0.020 mmol). The mixture was stirred at room temperature for 2 min, before N-tert-butyl-3-[[5-methyl-2-(4-piperazin-1-ylanilino)pyrimidin-4-yl]amino]benzenesulfonamide (10 mg, 0.020 mmol) was added. After the reaction mixture was stirred at 25° C. for 12 h, TLC showed the reaction was completed. The mixture was purified by reverse phase flash chromatography, followed by prep-TLC to give N-tert-butyl-3-[[2-[4-[4-[7-[[2-(2,6-dioxo-3-piperidyl)-1,3-dioxo-isoindolin-4-yl]amino]heptanoyl]piperazin-1-yl]anilino]-5-methyl-pyrimidin-4-yl]amino]benzenesulfonamide (13.5 mg, 76% yield). MS (ESI) m/z: 879.6 [M−H]⁻.

### Example 104. N-(tert-Butyl)-3-((2-((4-(4-(8-((2-(2,6-dioxopiperidin-3-yl)-1,3-dioxoisoindolin-4-yl)amino)octanoyl)piperazin-1-yl)phenyl)amino)-5-methylpyrimidin-4-yl)amino)benzenesulfonamide (JA-016)

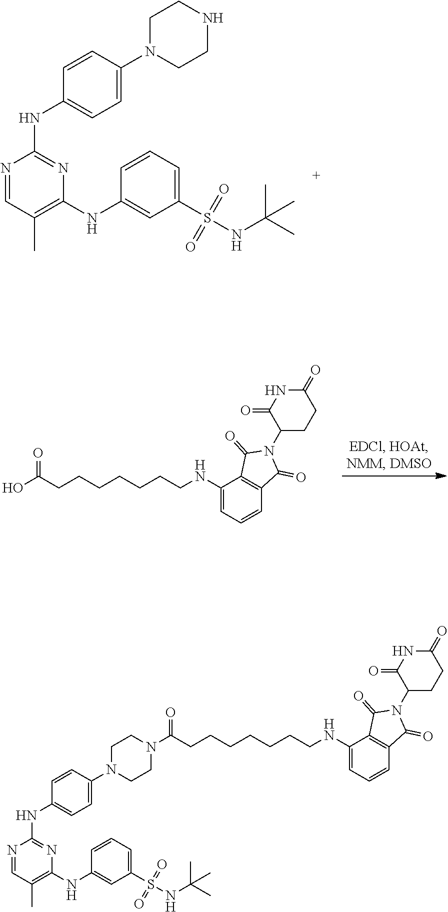 [SMILES: Cc1cnc(Nc2ccc(N3CCNCC3)cc2)nc1Nc1cccc(S(=O)(=O)NC(C)(C)C)c1 ; O=C(O)CCCCCCCNc1cccc2c1C(=O)N(C1CCC(=O)NC1=O)C2=O ; Cc1cnc(Nc2ccc(N3CCN(C(=O)CCCCCCCNc4cccc5c4C(=O)N(C4CCC(=O)NC4=O)C5=O)CC3)cc2)nc1Nc1cccc(S(=O)(=O)NC(C)(C)C)c1]

JA-016 was synthesized following the standard procedure for preparing JA-015 (17 mg, 94% yield). MS (ESI) m/z: 894.7 [M+H]⁺.

### Example 105. N-(tert-Butyl)-3-((2-((4-(4-(3-(2-((2-(2,6-dioxopiperidin-3-yl)-1,3-dioxoisoindolin-4-yl)amino)ethoxy)propanoyl)piperazin-1-yl)phenyl)amino)-5-methylpyrimidin-4-yl)amino)benzenesulfonamide (JA-017)

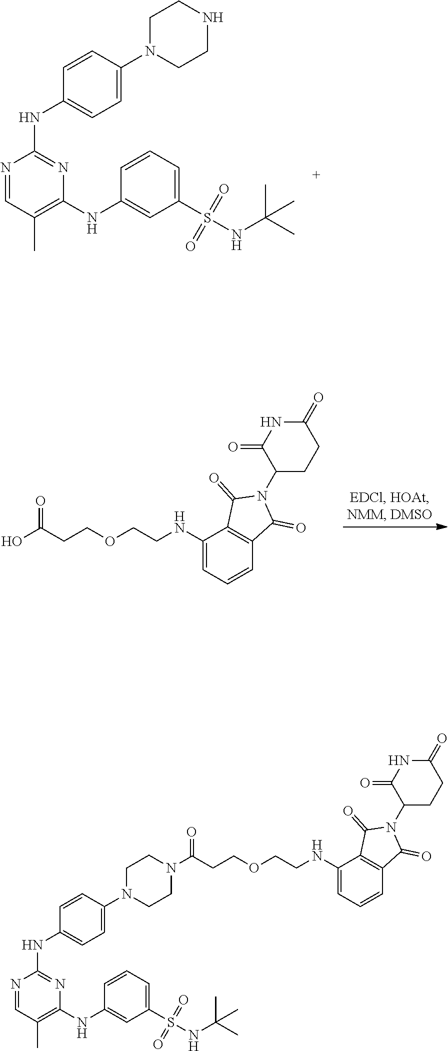 [SMILES: Cc1cnc(Nc2ccc(N3CCNCC3)cc2)nc1Nc1cccc(S(=O)(=O)NC(C)(C)C)c1 ; O=C(O)CCOCCNc1cccc2c1C(=O)N(C1CCC(=O)NC1=O)C2=O ; Cc1cnc(Nc2ccc(N3CCN(C(=O)CCOCCNc4cccc5c4C(=O)N(C4CCC(=O)NC4=O)C5=O)CC3)cc2)nc1Nc1cccc(S(=O)(=O)NC(C)(C)C)c1]

JA-017 was synthesized following the standard procedure for preparing JA-015 (16 mg, 98% yield). MS (ESI) m/z: 867.6 [M+H]⁺.

### Example 106. N-(tert-Butyl)-3-((2-((4-(4-(3-(2-(2-((2-(2,6-dioxopiperidin-3-yl)-1,3-dioxoisoindolin-4-yl)amino)ethoxy)ethoxy)propanoyl)piperazin-1-yl)phenyl)amino)-5-methylpyrimidin-4-yl)amino)benzenesulfonamide (JA-018)

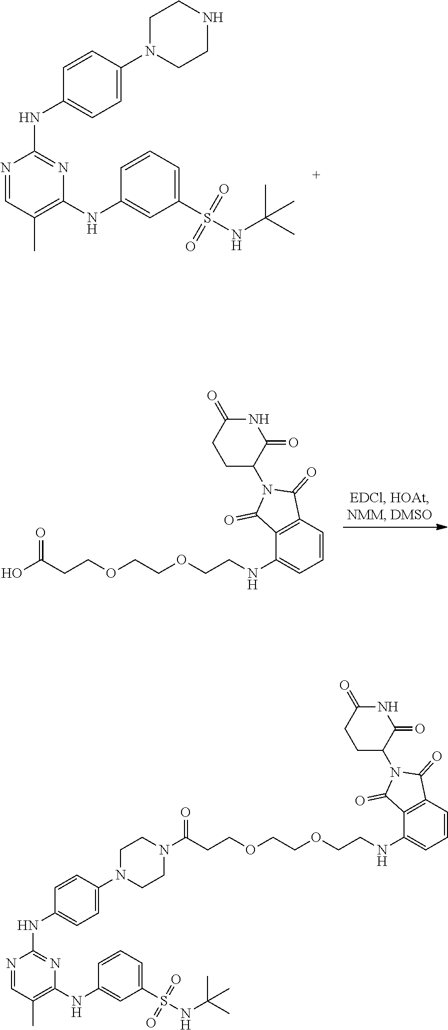 [SMILES: Cc1cnc(Nc2ccc(N3CCNCC3)cc2)nc1Nc1cccc(S(=O)(=O)NC(C)(C)C)c1 ; O=C(O)CCOCCOCCNc1cccc2c1C(=O)N(C1CCC(=O)NC1=O)C2=O ; Cc1cnc(Nc2ccc(N3CCN(C(=O)CCOCCOCCNc4cccc5c4C(=O)N(C4CCC(=O)NC4=O)C5=O)CC3)cc2)nc1Nc1cccc(S(=O)(=O)NC(C)(C)C)c1]

JA-018 was synthesized following the standard procedure for preparing JA-015 (15.8 mg, 92% yield). MS (ESI) m/z: 911.6 [M+H]⁺.

### Example 107. N-(tert-Butyl)-3-((2-((4-(4-(3-(2-(2-(2-((2-(2,6-dioxopiperidin-3-yl)-1,3-dioxoisoindolin-4-yl)amino)ethoxy)ethoxy)ethoxy)propanoyl)piperazin-1-yl)phenyl)amino)-5-methylpyrimidin-4-yl)amino)benzenesulfonamide (JA-019)

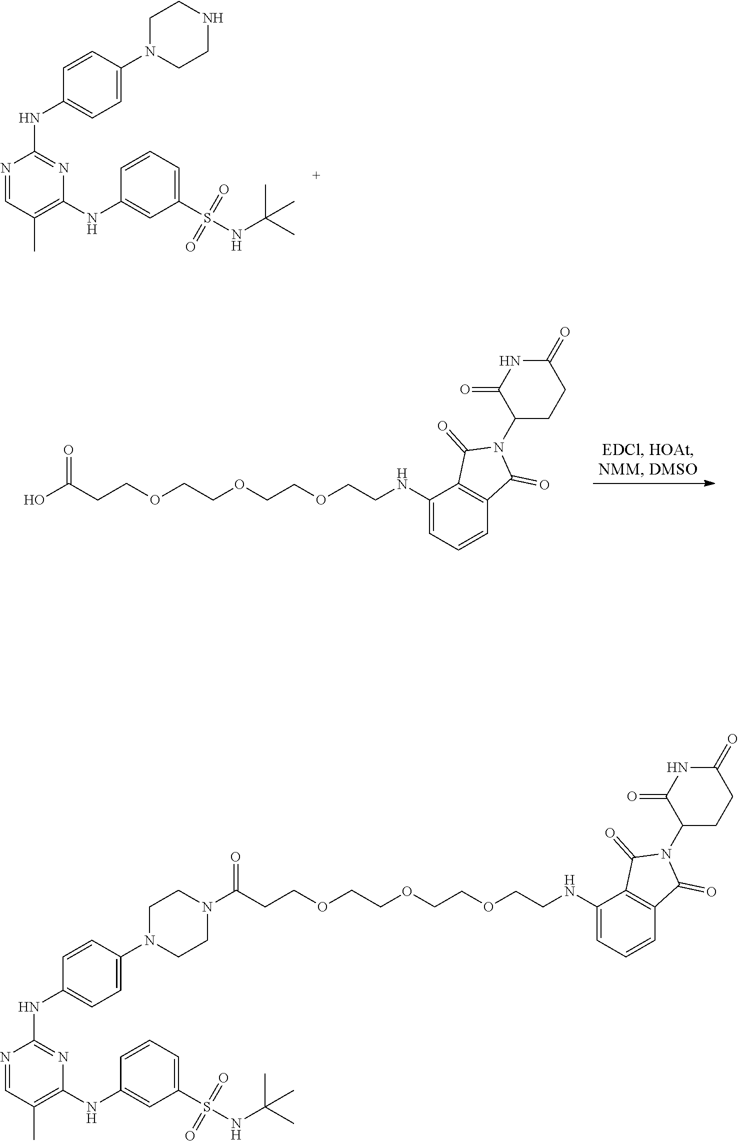 [SMILES: Cc1cnc(Nc2ccc(N3CCNCC3)cc2)nc1Nc1cccc(S(=O)(=O)NC(C)(C)C)c1 ; O=C(O)CCOCCOCCOCCNc1cccc2c1C(=O)N(C1CCC(=O)NC1=O)C2=O ; Cc1cnc(Nc2ccc(N3CCN(C(=O)CCOCCOCCOCCNc4cccc5c4C(=O)N(C4CCC(=O)NC4=O)C5=O)CC3)cc2)nc1Nc1cccc(S(=O)(=O)NC(C)(C)C)c1]

JA-019 was synthesized following the standard procedure for preparing JA-015 (16.3 mg, 91% yield). MS (ESI) m/z: 956.9 [M+H]⁺.

### Example 108. N-(tert-Butyl)-3-((2-((4-(4-(1-((2-(2,6-dioxopiperidin-3-yl)-1,3-dioxoisoindolin-4-yl)amino)-3,6,9,12-tetraoxapentadecan-15-oyl)piperazin-1-yl)phenyl)amino)-5-methylpyrimidin-4-yl)amino)benzenesulfonamide (JA-020)

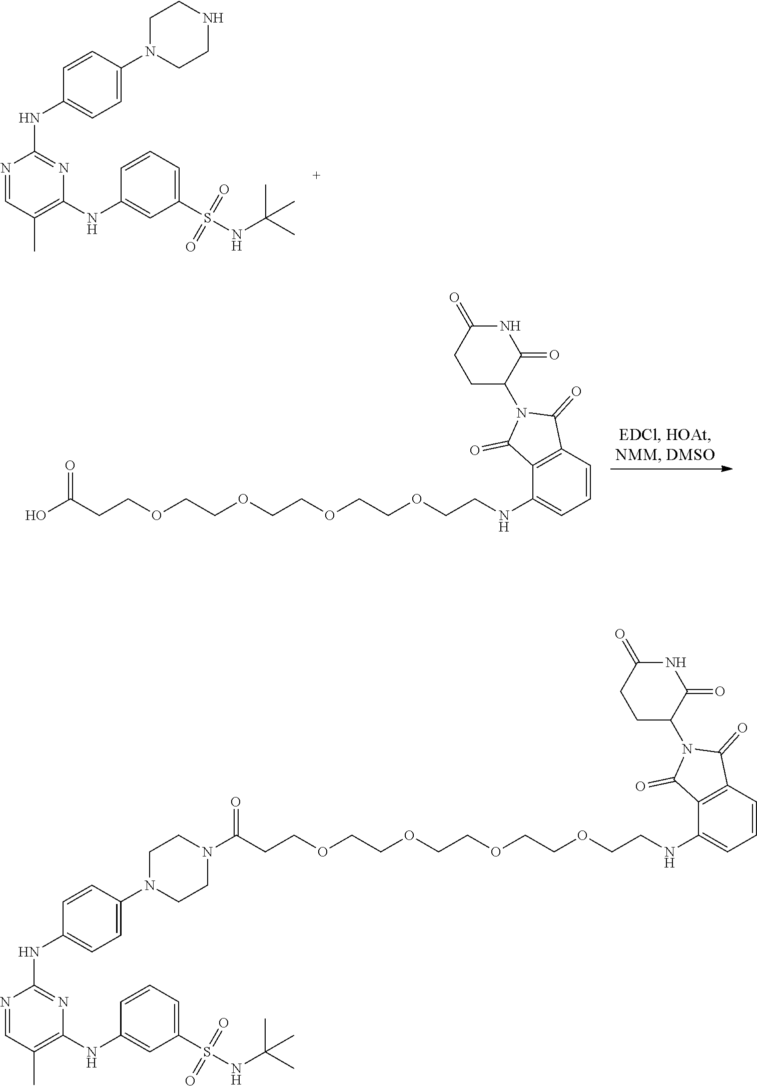

JA-020 was synthesized following the standard procedure for preparing JA-015 (15.4 mg, 82% yield). MS (ESI) m/z: 1000.8 [M+H]⁺.

### Example 109. N-(tert-Butyl)-3-((-(4-(4-(1-((2-(2,6-dioxopiperidin-3-yl)-1,3-dioxoisoindolin-4-yl)amino)-3,6,9,12,15-pentaoxaoctadecan-18-oyl)piperazin-1l-yl)phenyl)amino)-5-methylpyrimidin-4-yl)amino)benzenesulfonamide (JA-021)

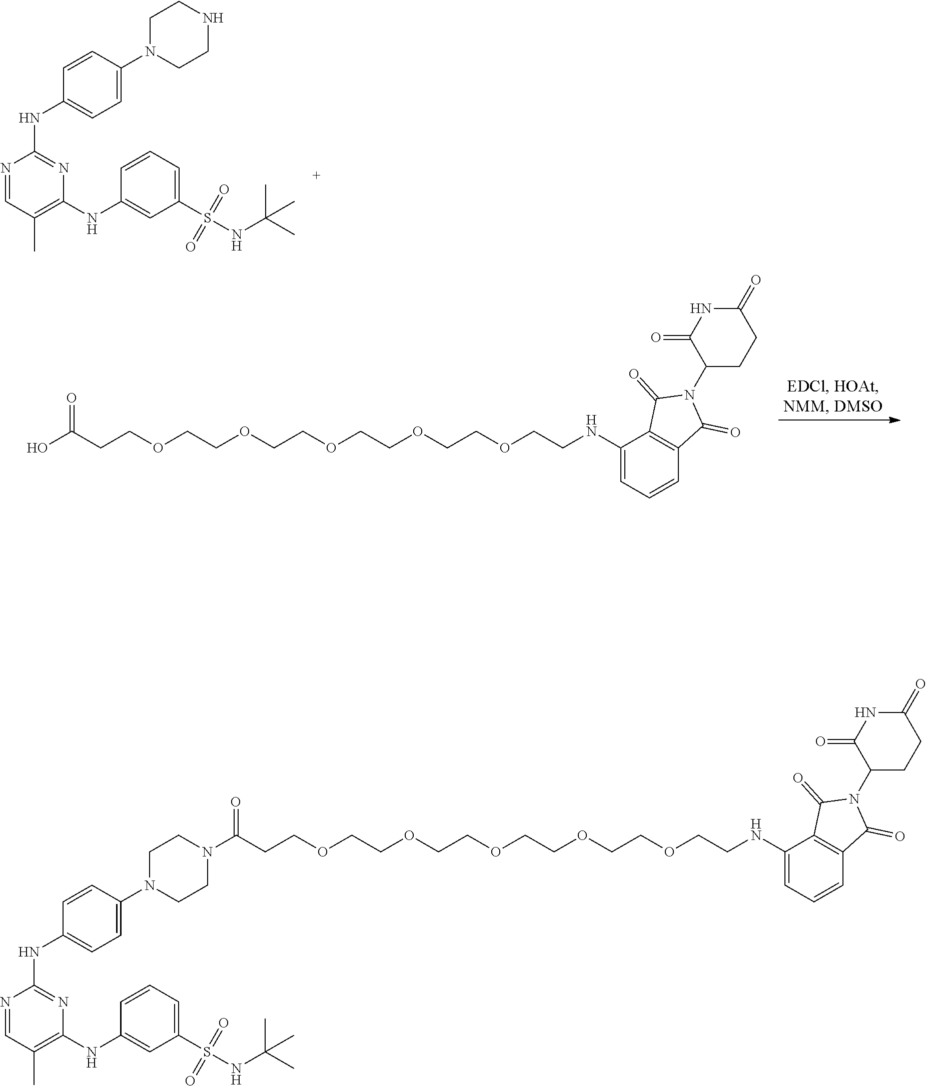 [SMILES: Cc1cnc(Nc2ccc(N3CCNCC3)cc2)nc1Nc1cccc(S(=O)(=O)NC(C)(C)C)c1 ; O=C(O)CCOCCOCCOCCOCCOCCNc1cccc2c1C(=O)N(C1CCC(=O)NC1=O)C2=O ; Cc1cnc(Nc2ccc(N3CCN(C(=O)CCOCCOCCOCCOCCOCCNc4cccc5c4C(=O)N(C4CCC(=O)NC4=O)C5=O)CC3)cc2)nc1Nc1cccc(S(=O)(=O)NC(C)(C)C)c1]

JA-021 was synthesized following the standard procedure for preparing JA-015 (13.8 mg, 70% yield). MS (ESI) m/z: 1043.5 [M+H]⁺.

### Example 110. (2S,4R)-1-((S)-2-(4-(4-(4-((4-((3-(N-(tert-Butyl)sulfamoyl)phenyl)amino)-5-methylpyrimidin-2-yl)amino)phenyl)piperazin-1-yl)-4-oxobutanamido)-3,3-dimethylbutanoyl)-4-hydroxy-N-(4-(4-methylthiazol-5-yl)benzyl)pyrrolidine-2-carboxamide (JA-022)

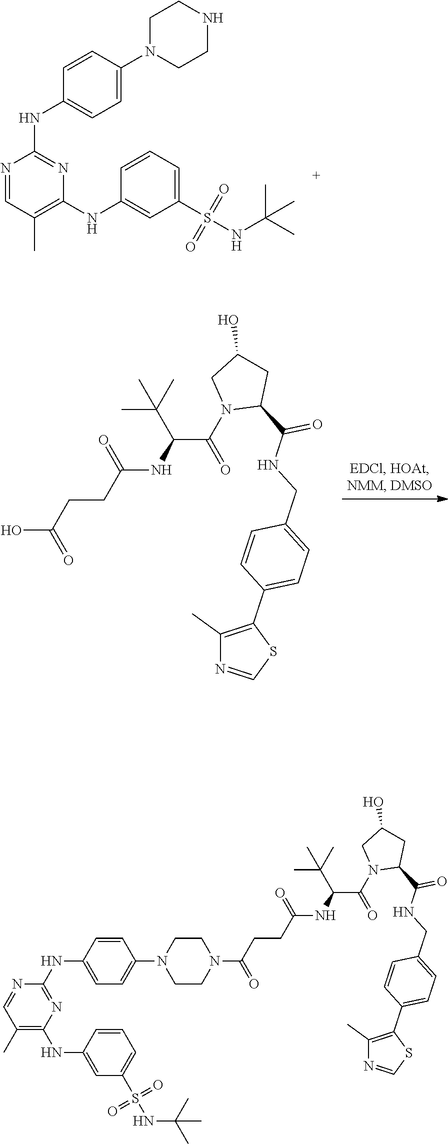 [SMILES: Cc1cnc(Nc2ccc(N3CCNCC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCC(=O)O)C(C)(C)C)cc1 ; Cc1cnc(Nc2ccc(N3CCN(C(=O)CCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-022 was synthesized following the standard procedure for preparing JA-015 (17.7 mg, 93% yield). MS (ESI) m/z: 1009.8 [M+H]⁺.

### Example 111. (2S,4R)-1-((S)-2-(5-(4-(4-((4-((3-(N-(tert-Butyl)sulfamoyl)phenyl)amino)-5-methylpyrimidin-2-yl)amino)phenyl)piperazin-1-yl)-5-oxopentanamido)-3,3-dimethylbutanoyl)-4-hydroxy-N-(4-(4-methylthiazol-5-yl)benzyl)pyrrolidine-2-carboxamide (JA-023)

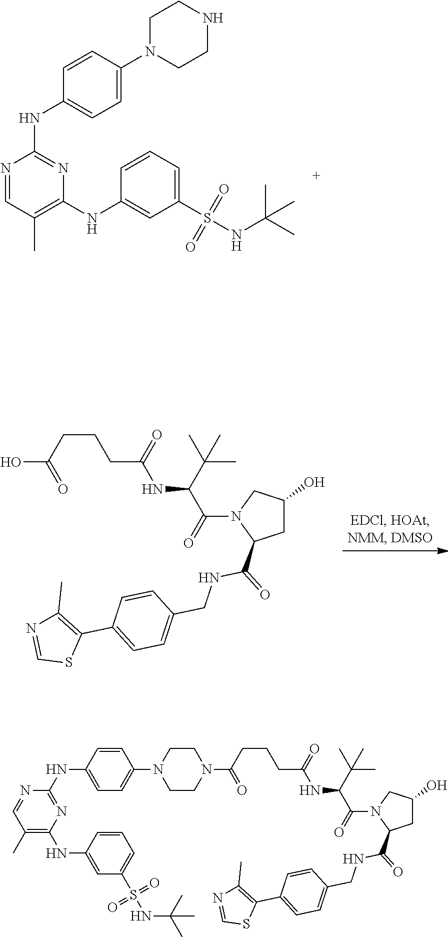 [SMILES: Cc1cnc(Nc2ccc(N3CCNCC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCCC(=O)O)C(C)(C)C)cc1 ; Cc1cnc(Nc2ccc(N3CCN(C(=O)CCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-023 was synthesized following the standard procedure for preparing JA-015 (6.3 mg, 33% yield). MS (ESI) m/z: 1023.6 [M+H]⁺.

### Example 112. (2S,4R)-1-((S)-2-(6-(4-(4-((4-((3-(N-(tert-Butyl)sulfamoyl)phenyl)amino)-5-methylpyrimidin-2-yl)amino)phenyl)piperazin-1-yl)-6-oxohexanamido)-3,3-dimethylbutanoyl)-4-hydroxy-N-(4-(4-methylthiazol-5-yl)benzyl)pyrrolidine-2-carboxamide (JA-024)

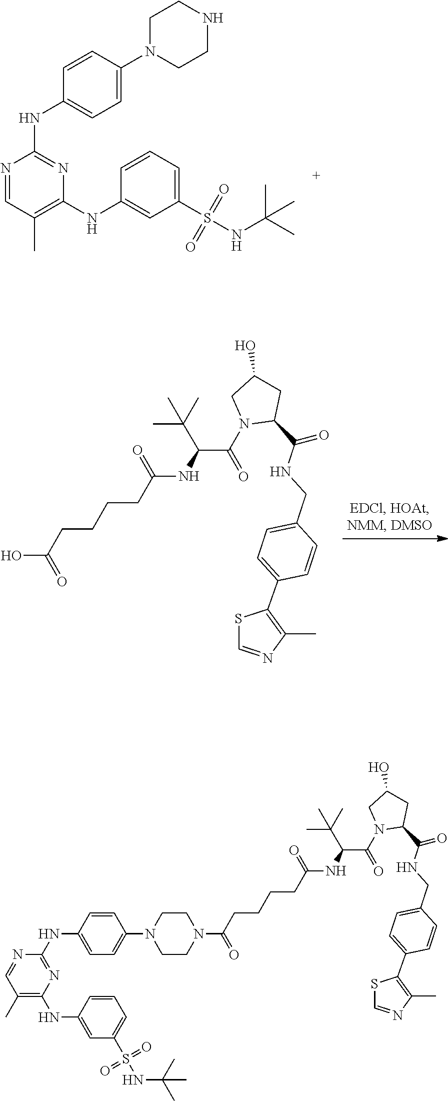 [SMILES: Cc1cnc(Nc2ccc(N3CCNCC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCCCC(=O)O)C(C)(C)C)cc1 ; Cc1cnc(Nc2ccc(N3CCN(C(=O)CCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-024 was synthesized following the standard procedure for preparing JA-015 (19 mg, 97% yield). MS (ESI) m/z: 1036.7 [M+H]⁺.

### Example 113. (2S,4R)-1-((S)-2-(7-(4-(4-((4-((3-(N-(tert-Butyl)sulfamoyl)phenyl)amino)-5-methylpyrimidin-2-yl)amino)phenyl)piperazin-1-yl)-7-oxoheptanamido)-3,3-dimethylbutanoyl)-4-hydroxy-N-(4-(4-methylthiazol-5-yl)benzyl)pyrrolidine-2-carboxamide (JA-025)

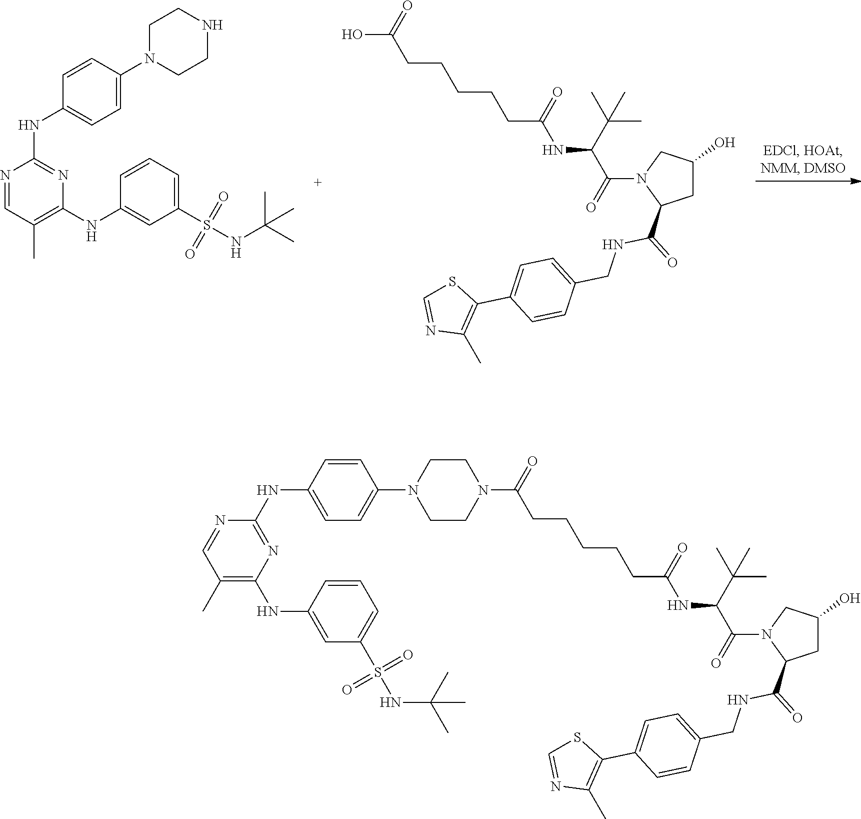 [SMILES: Cc1cnc(Nc2ccc(N3CCNCC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCCCCC(=O)O)C(C)(C)C)cc1 ; Cc1cnc(Nc2ccc(N3CCN(C(=O)CCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-025 was synthesized following the standard procedure for preparing JA-015 (18.9 mg, 96% yield). MS (ESI) m/z: 1050.6 [M+H]⁺.

### Example 114. (2S,4R)-1-((S)-2-(8-(4-(4-((4-((3-(N-(tert-Butyl)sulfamoyl)phenyl)amino)-5-methylpyrimidin-2-yl)amino)phenyl)piperazin-1-yl)-8-oxooctanamido)-3,3-dimethylbutanoyl)-4-hydroxy-N-(4-(4-methylthiazol-5-yl)benzyl)pyrrolidine-2-carboxamide (JA-026)

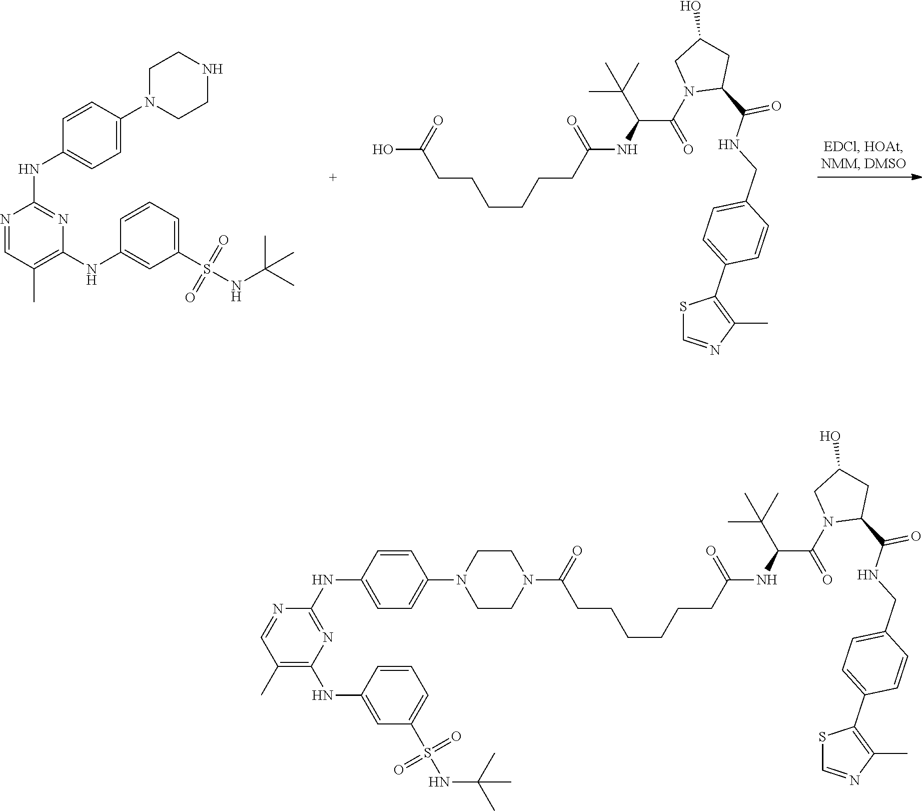 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCCCCCC(=O)O)C(C)(C)C)cc1 ; Cc1cnc(Nc2ccc(N3CCN(C(=O)CCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-026 was synthesized following the standard procedure for preparing JA-015 (19.2 mg, 96% yield). MS (ESI) m/z: 1065.9 [M+H]⁺.

### Example 115. (2S,4R)-1-((S)-2-(9-(4-(4-((4-((3-(N-(tert-Butyl)sulfamoyl)phenyl)amino)-5-methylpyrimidin-2-yl)amino)phenyl)piperazin-1-yl)-9-oxononanamido)-3,3-dimethylbutanoyl)-4-hydroxy-N-(4-(4-methylthiazol-5-yl)benzyl)pyrrolidine-2-carboxamide (JA-027)

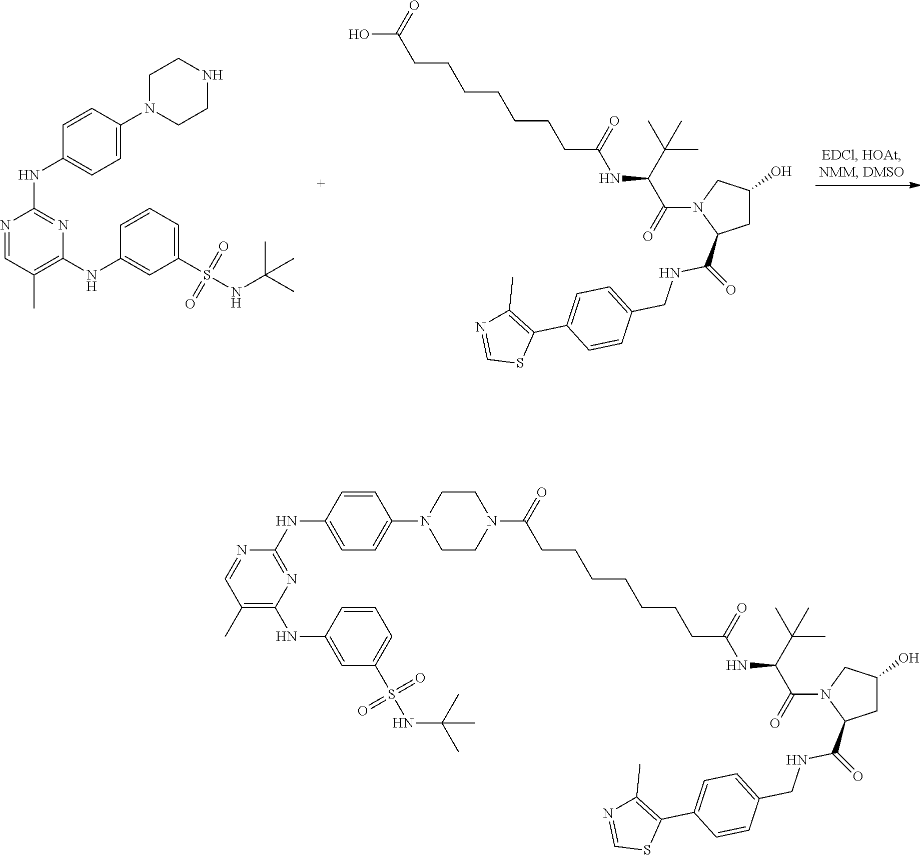 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCCCCCCC(=O)O)C(C)(C)C)cc1 ; Cc1cnc(Nc2ccc(N3CCN(C(=O)CCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-027 was synthesized following the standard procedure for preparing JA-015 (18.3 mg, 90% yield). MS (ESI) m/z: 1078.7 [M+H]⁺.

### Example 116. (2S,4R)-1-((S)-2-(10-(4-(4-((4-((3-(N-(tert-Butyl)sulfamoyl)phenyl)amino)-5-methylpyrimidin-2-yl)amino)phenyl)piperazin-1-yl)-10-oxodecanamido)-3,3-dimethylbutanoyl)-4-hydroxy-N-(4-(4-methylthiazol-5-yl)benzyl)pyrrolidine-2-carboxamide (JA-028)

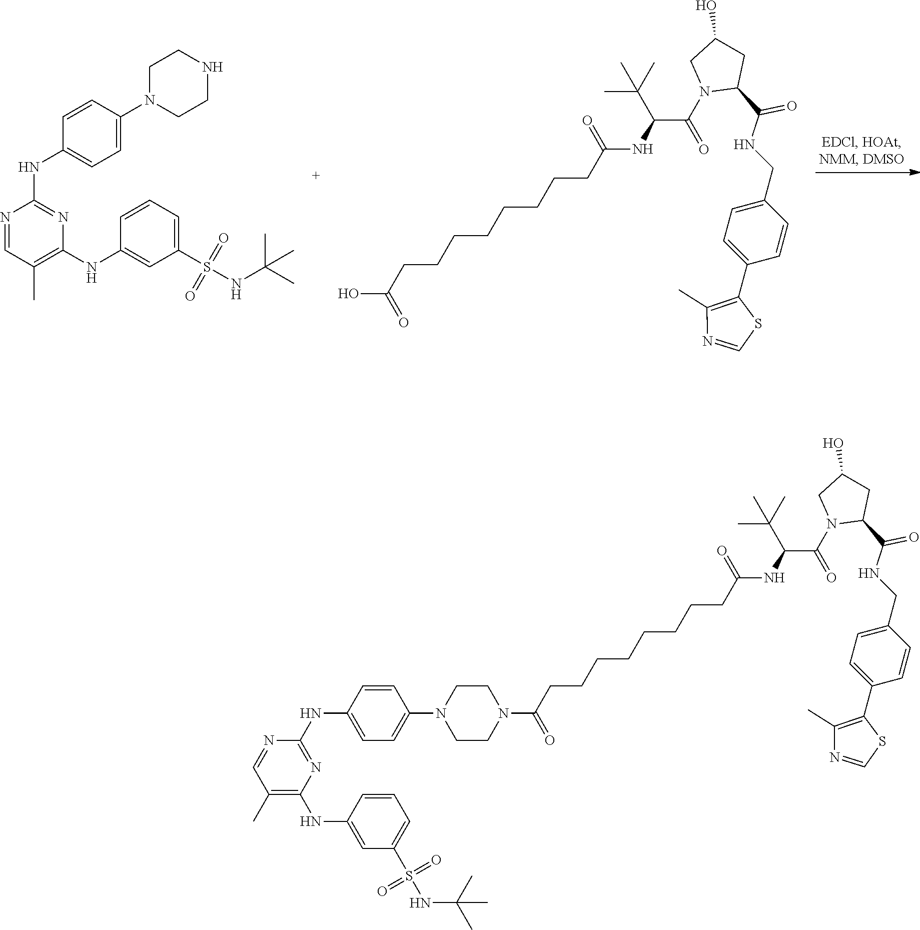 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCCCCCCCC(=O)O)C(C)(C)C)cc1 ; Cc1cnc(Nc2ccc(N3CCN(C(=O)CCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-028 was synthesized following the standard procedure for preparing JA-015 (18.5 mg, 90% yield). MS (ESI) m/z: 1093.9 [M+H]⁺.

### Example 117. (2S,4R)-1-((S)-2-(11-(4-(4-((4-((3-(N-(tert-Butyl)sulfamoyl)phenyl)amino)-5-methylpyrimidin-2-yl)amino)phenyl)piperazin-1-yl)-11-oxoundecanamido)-3,3-dimethylbutanoyl)-4-hydroxy-N-(4-(4-methylthiazol-5-yl)benzyl)pyrrolidine-2-carboxamide (JA-029)

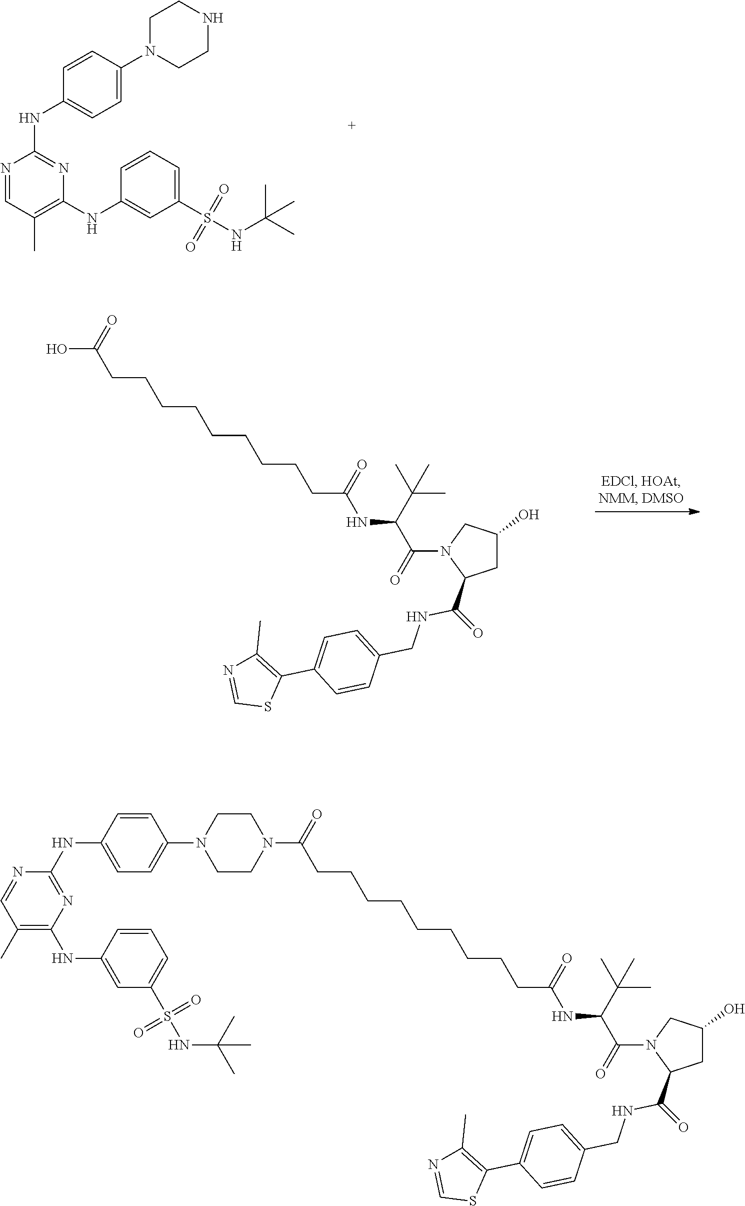 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; Cc1cnc(Nc2ccc(N3CCN(C(=O)CCCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-029 was synthesized following the standard procedure for preparing JA-015 (18.6 mg, 89% yield). MS (ESI) m/z: 1107.6 [M+H]⁺.

### Example 118. (2S,4R)-1-((S)-2-(2-(2-(4-(4-((4-((3-(N-(tert-Butyl)sulfamoyl)phenyl)amino)-5-methylpyrimidin-2-yl)amino)phenyl)piperazin-1-yl)-2-oxoethoxy)acetamido)-3,3-dimethylbutanoyl)-4-hydroxy-N-(4-(4-methylthiazol-5-yl)benzyl)pyrrolidine-2-carboxamide (JA-030)

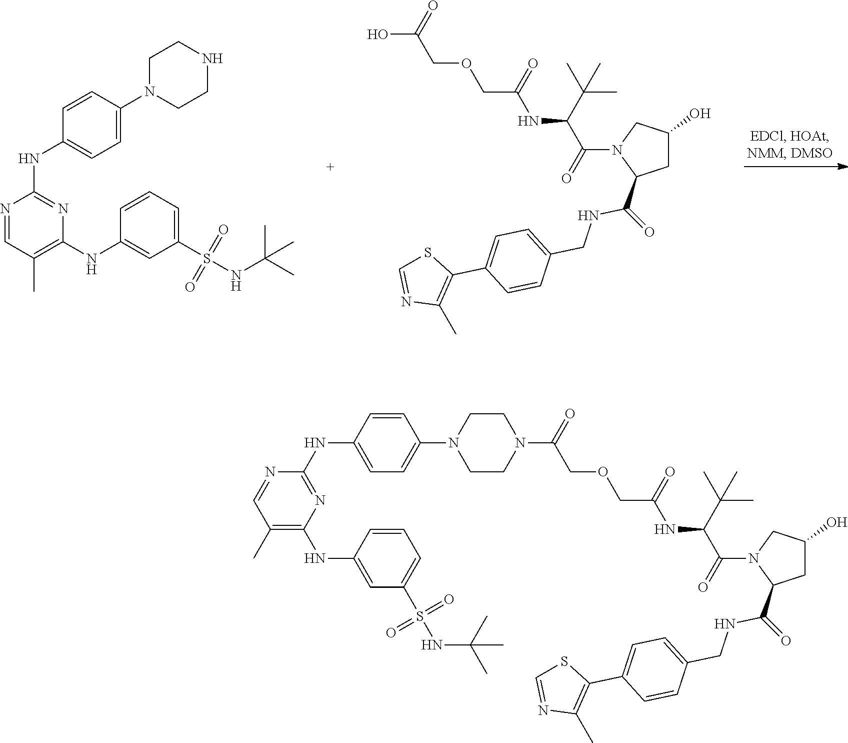 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)COCC(=O)O)C(C)(C)C)cc1 ; Cc1cnc(Nc2ccc(N3CCN(C(=O)COCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-030 was synthesized following the standard procedure for preparing JA-015 (6 mg, 29% yield). MS (ESI) m/z: 1025.7 [M+H]⁺.

### Example 119. (2S,4R)-1-((S)-2-(3-(3-(4-(4-((4-((3-(N-(tert-Butyl)sulfamoyl)phenyl)amino)-5-methylpyrimidin-2-yl)amino)phenyl)piperazin-1-yl)-3-oxopropoxy)propanamido)-3,3-dimethylbutanoyl)-4-hydroxy-N-(4-(4-methylthiazol-5-yl)benzyl)pyrrolidine-2-carboxamide (JA-031)

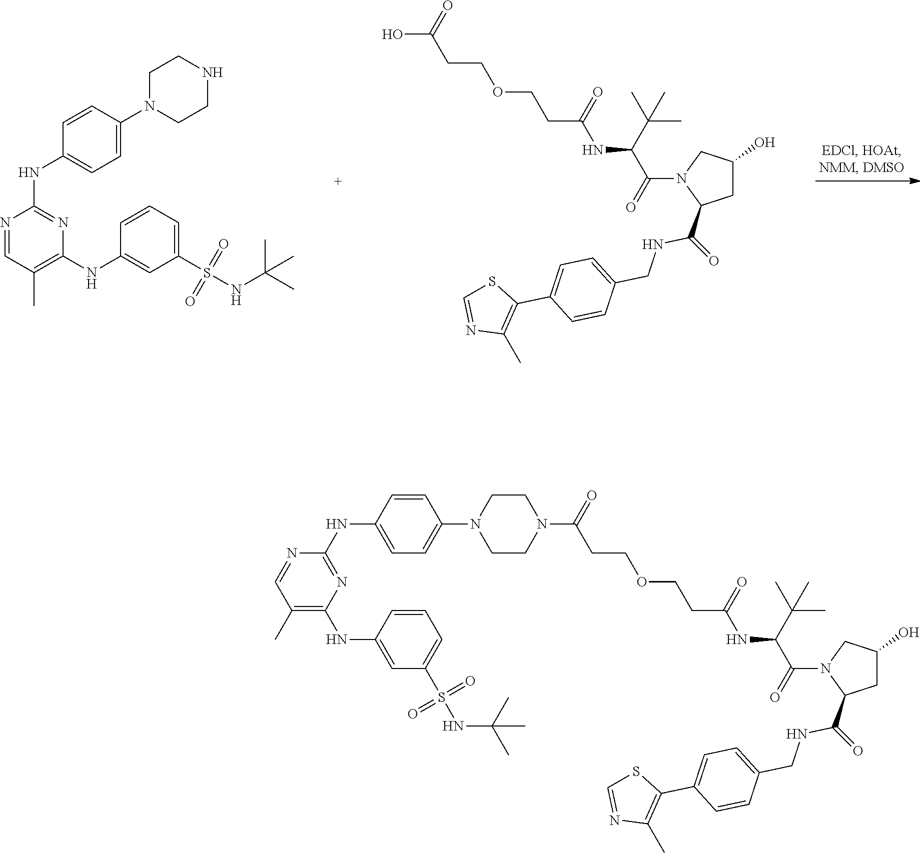 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1cnc(Nc2ccc(N3CCN(C(=O)CCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-031 was synthesized following the standard procedure for preparing JA-015 (7 mg, 33% yield). MS (ESI) m/z: 1053.9 [M+H]⁺.

### Example 120. (2S,4R)-1-((S)-2-(2-(2-(2-(4-(4-((4-((3-(N-(tert-Butyl)sulfamoyl)phenyl)amino)-5-methylpyrimidin-2-yl)amino)phenyl)piperazin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide (JA-032)

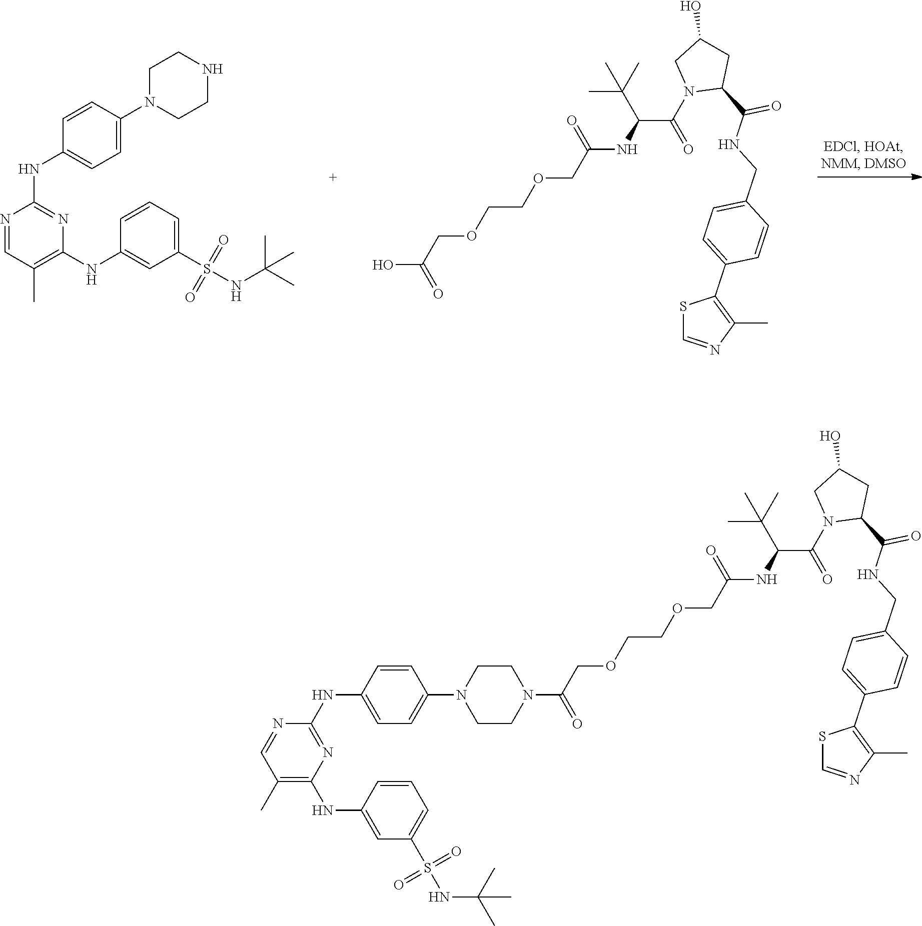 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)COCCOCC(=O)O)C(C)(C)C)cc1 ; Cc1cnc(Nc2ccc(N3CCN(C(=O)COCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-032 was synthesized following the standard procedure for preparing JA-015 (7 mg, 32% yield). MS (ESI) m/z: 1069.8 [M+H]⁺.

### Example 121. (2S,4R)-1-((S)-2-(3-(2-(3-(4-(4-((4-((3-(N-(tert-Butyl)sulfamoyl)phenyl)amino)-5-methylpyrimidin-2-yl)amino)phenyl)piperazin-1-yl)-3-oxopropoxy)ethoxy)propanamido)-3,3-dimethylbutanoyl)-4-hydroxy-N-(4-(4-methylthiazol-5-yl)benzyl)pyrrolidine-2-carboxamide (JA-033)

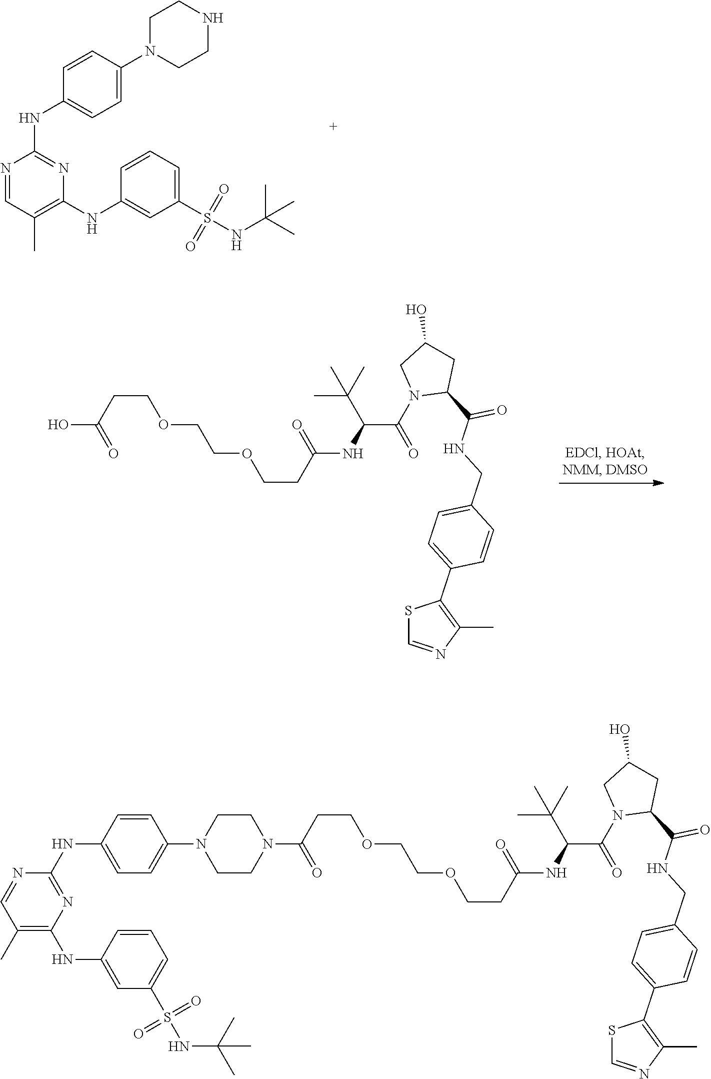 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; Cc1cnc(Nc2ccc(N3CCN(C(=O)CCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-033 was synthesized following the standard procedure for preparing JA-015 (5 mg, 23% yield). MS (ESI) m/z: 1097.6 [M+H]⁺.

### Example 122. (2S,4R)-1-((S)-2-(tert-Butyl)-16-(4-(4-((4-((3-(N-(tert-butyl)sulfamoyl)phenyl)amino)-5-methylpyrimidin-2-yl)amino)phenyl)piperazin-1-yl)-4,16-dioxo-7,10,13-trioxa-3-azahexadecanoyl)-4-hydroxy-N-(4-(4-methylthiazol-5-yl)benzyl)pyrrolidine-2-carboxamide (JA-034)

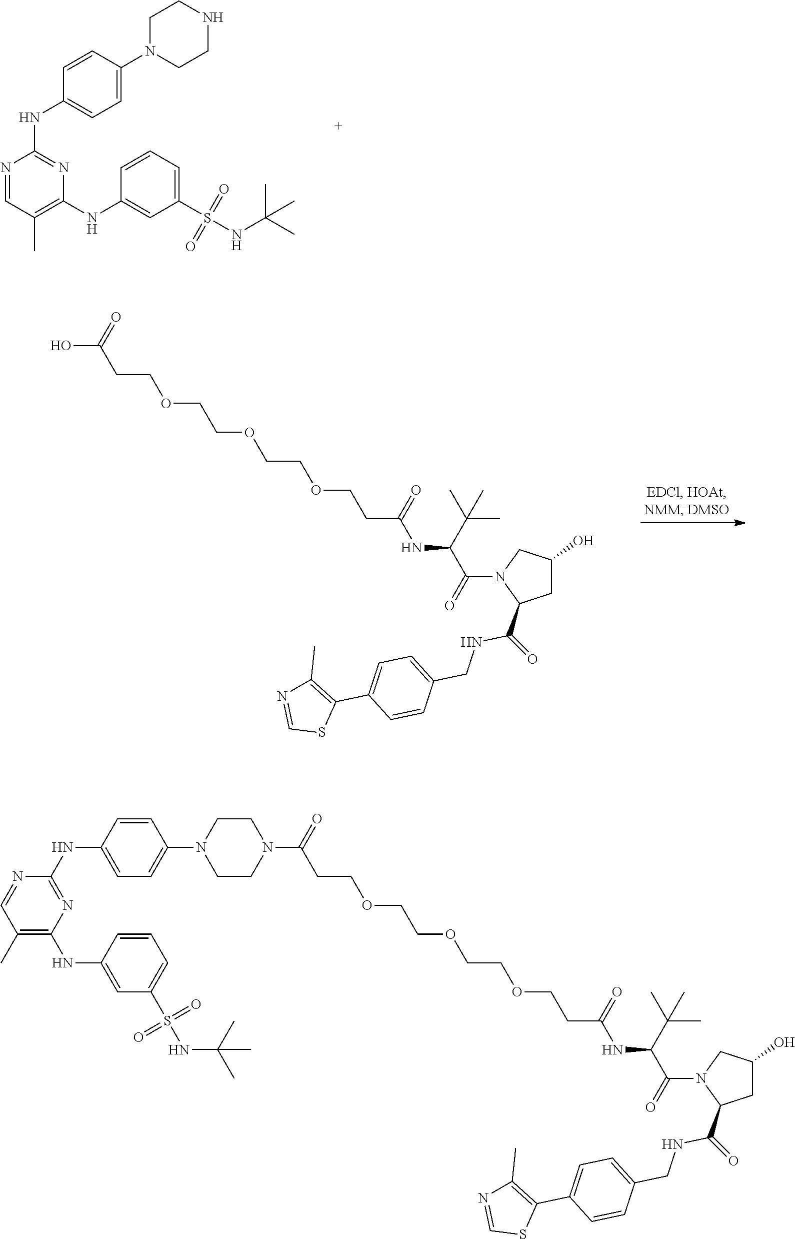 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCOCCOCCOCCC(=O)O)C(C)(C)C)cc1 ; Cc1cnc(Nc2ccc(N3CCN(C(=O)CCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-034 was synthesized following the standard procedure for preparing JA-015 (6 mg, 26% yield). MS (ESI) m/z: 1141.1 [M+H]⁺.

### Example 123. (2S,4R)-1-((S)-2-(tert-Butyl)-19-(4-(4-((4-((3-(N-(tert-butyl)sulfamoyl)phenyl)amino)-5-methylpyrimidin-2-yl)amino)phenyl)piperazin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide (JA-035)

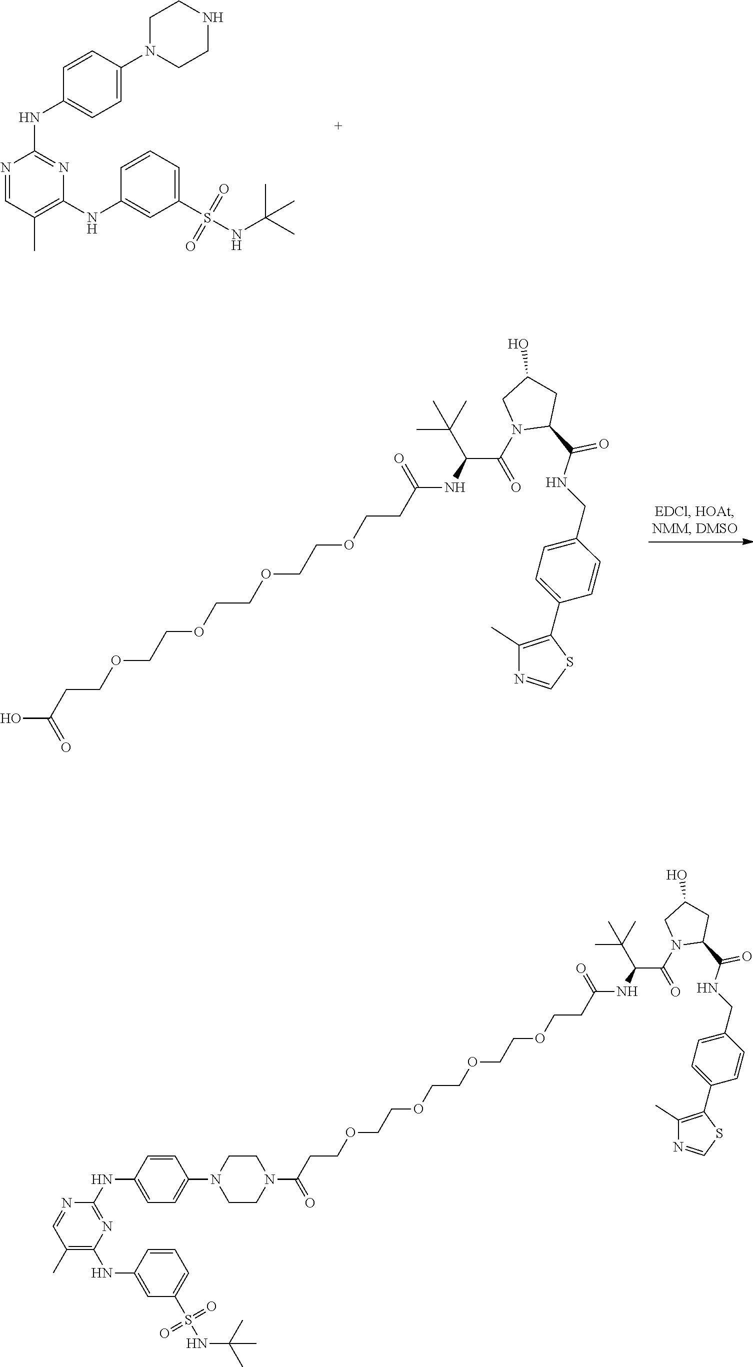 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; Cc1cnc(Nc2ccc(N3CCN(C(=O)CCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-035 was synthesized following the standard procedure for preparing JA-015 (7 mg, 29% yield). MS (ESI) m/z: 1185.1 [M+H]⁺.

### Example 124. (2S,4R)-1-((S)-2-(tert-Butyl)-20-(4-(4-((4-((3-(N-(tert-butyl)sulfamoyl)phenyl)amino)-5-methylpyrimidin-2-yl)amino)phenyl)piperazin-1-yl)-4,20-dioxo-6,9,12,15,18-pentaoxa-3-azaicosanoyl)-4-hydroxy-N-(4-(4-methylthiazol-5-yl)benzyl)pyrrolidine-2-carboxamide (JA-036)

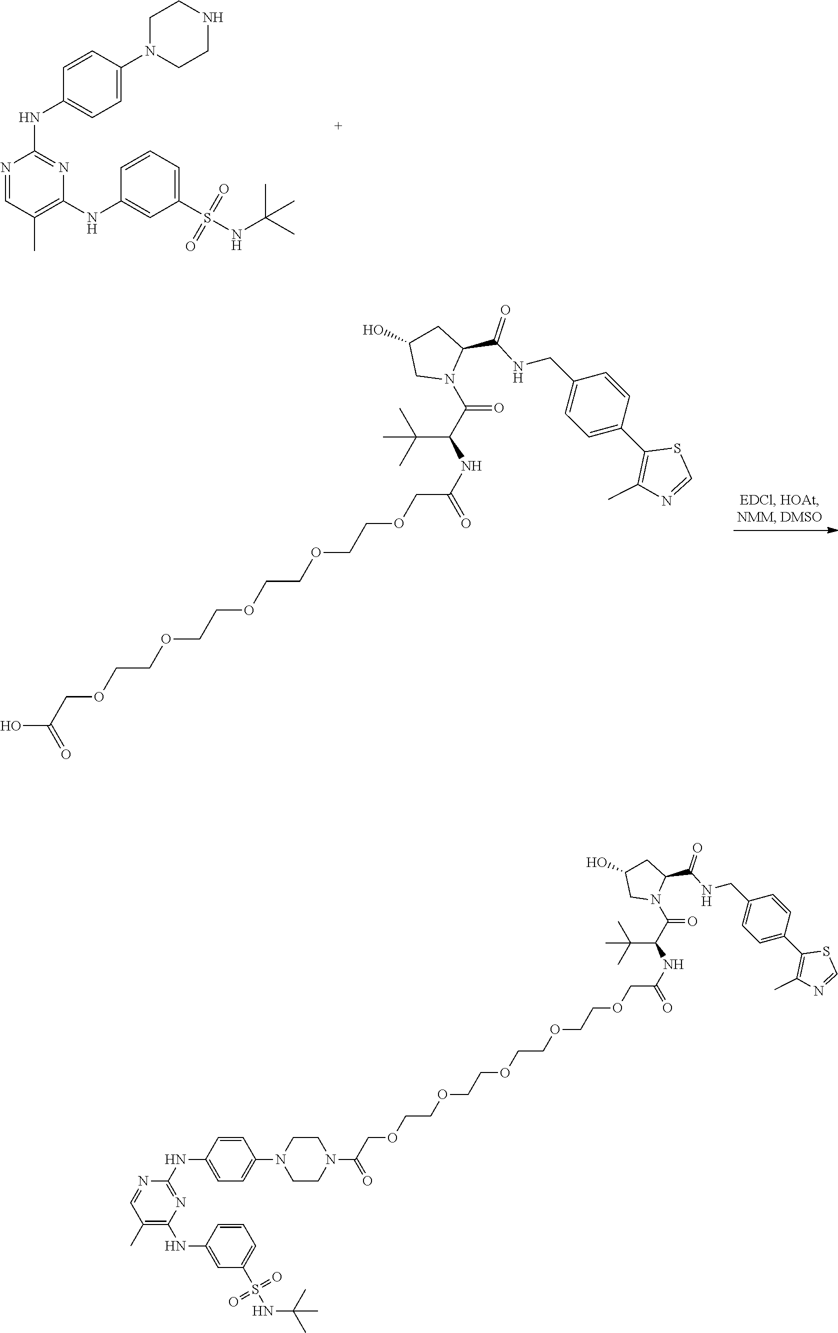 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; Cc1cnc(Nc2ccc(N3CCN(C(=O)COCCOCCOCCOCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-036 was synthesized following the standard procedure for preparing JA-015 (7 mg, 29% yield). MS (ESI) m/z: 1201.9 [M+H]⁺.

### Example 125. (2S,4R)-1-((S)-2-(tert-Butyl)-22-(4-(4-((4-((3-(N-(tert-butyl)sulfamoyl)phenyl)amino)-5-methylpyrimidin-2-yl)amino)phenyl)piperazin-1-yl)-4,22-dioxo-7,10,13,16,19-pentaoxa-3-azadocosanoyl)-4-hydroxy-N-(4-(4-methylthiazol-5-yl)benzyl)pyrrolidine-2-carboxamide (JA-037)

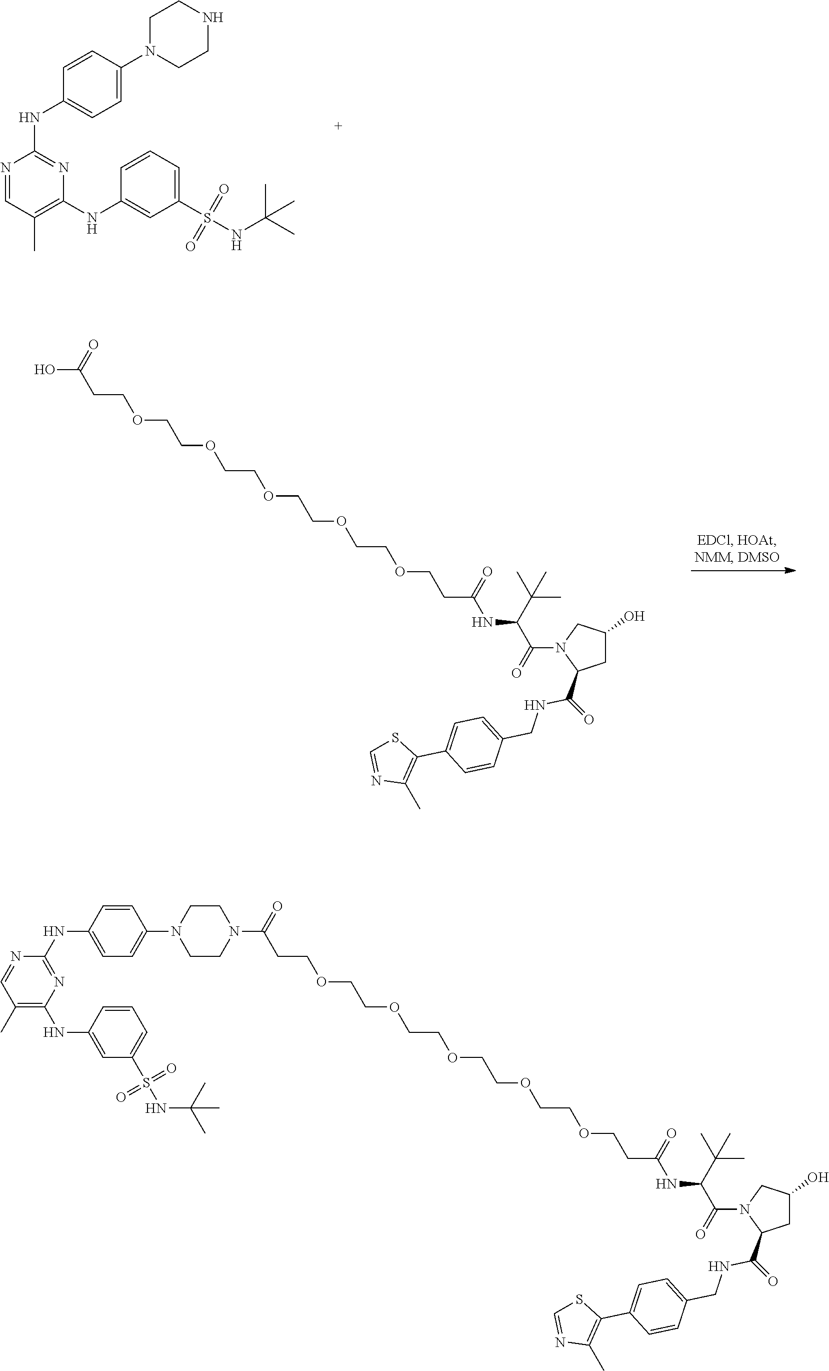 [SMILES: Cc1cnc(Nc2ccc(N3CCNCC3)cc2)nc1Nc1cccc(S(=O)(=O)NC(C)(C)C)c1 ; "+" ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; Cc1cnc(Nc2ccc(N3CCN(C(=O)CCOCCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-037 was synthesized following the standard procedure for preparing JA-015 (7 mg, 28% yield). MS (ESI) m/z: 1229.1 [1\4+H]+.

### Example 126. 4-[[8-[4-[4-[8-[3,5-Difluoro-4-(morpholinomethyl)phenyl]quinoxalin-2-yl]pyrazol-1-yl]-1-piperidyl]-8-oxo-octyl]amino]-2-(2,6-dioxo-3-piperidyl)isoindoline-1,3-dione (JA-038)

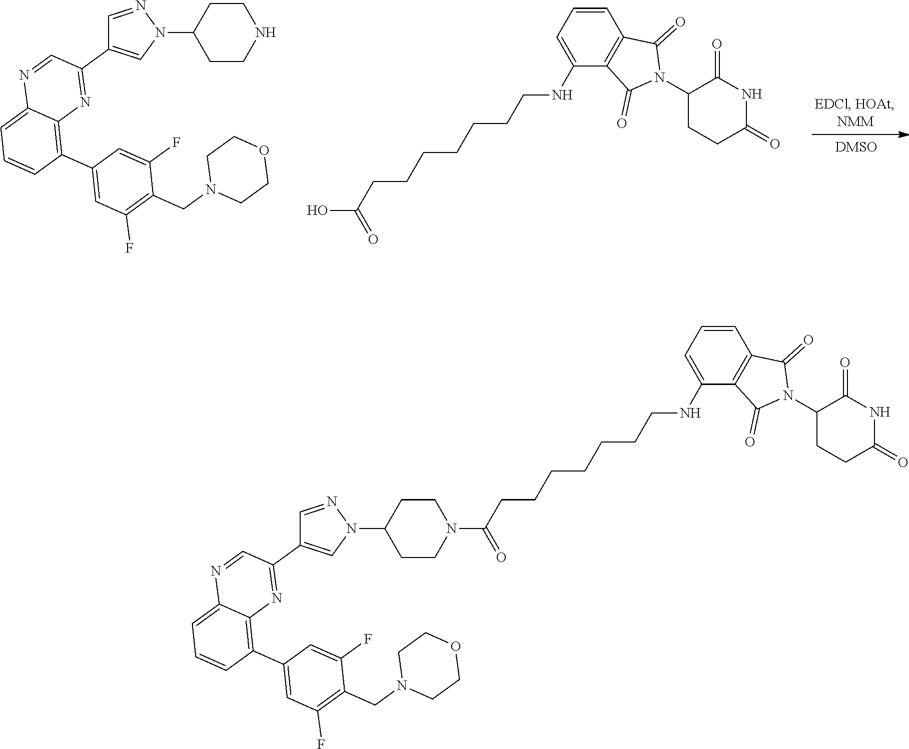 [SMILES: Fc1cc(-c2cccc3ncc(-c4cnn(C5CCNCC5)c4)nc23)cc(F)c1CN1CCOCC1 ; O=C(O)CCCCCCCNc1cccc2c1C(=O)N(C1CCC(=O)NC1=O)C2=O ; O=C1CCC(N2C(=O)c3cccc(NCCCCCCCC(=O)N4CCC(n5cc(-c6cnc7cccc(-c8cc(F)c(CN9CCOCC9)c(F)c8)c7n6)cn5)CC4)c3C2=O)C(=O)N1]

JA-038 was synthesized following the standard procedure for preparing JA-001 (6.8 mg, 46.9% yield). MS (ESI) m/z: 888.4 [M+H]+.

### Example 127. 4-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethylamino]-2-(2,6-dioxo-3-piperidyl)isoindoline-1,3-dione (JA-039)

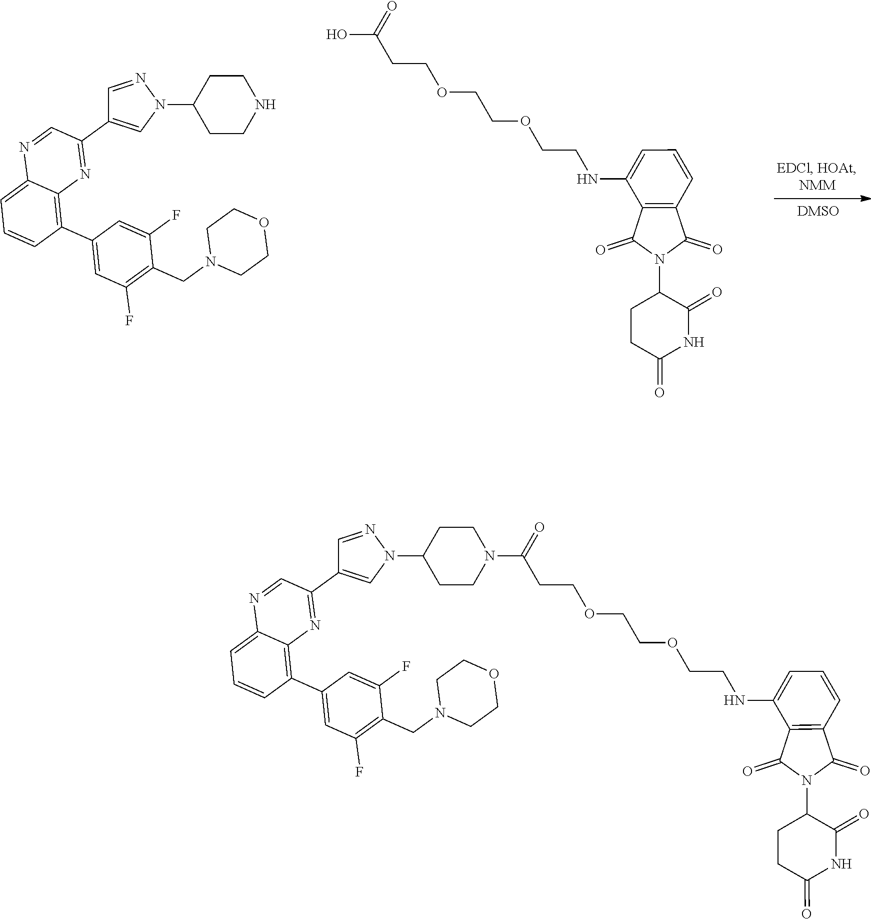 [SMILES: Fc1cc(-c2cccc3ncc(-c4cnn(C5CCNCC5)c4)nc23)cc(F)c1CN1CCOCC1 ; O=C(O)CCOCCOCCNc1cccc2c1C(=O)N(C1CCC(=O)NC1=O)C2=O ; O=C1CCC(N2C(=O)c3cccc(NCCOCCOCCC(=O)N4CCC(n5cc(-c6cnc7cccc(-c8cc(F)c(CN9CCOCC9)c(F)c8)c7n6)cn5)CC4)c3C2=O)C(=O)N1]

JA-039 was synthesized following the standard procedure for preparing JA-001 (7.2 mg, 48.7% yield). MS (ESI) m/z: 906.4 [M+H]⁺.

### Example 128. 4-[[7-[4-[4-[8-[3,5-Difluoro-4-(morpholinomethyl)phenyl]quinoxalin-2-yl]pyrazol-1-yl]-1-piperidyl]-7-oxo-heptyl]amino]-2-(2,6-dioxo-3-piperidyl)isoindoline-1,3-dione (JA-040)

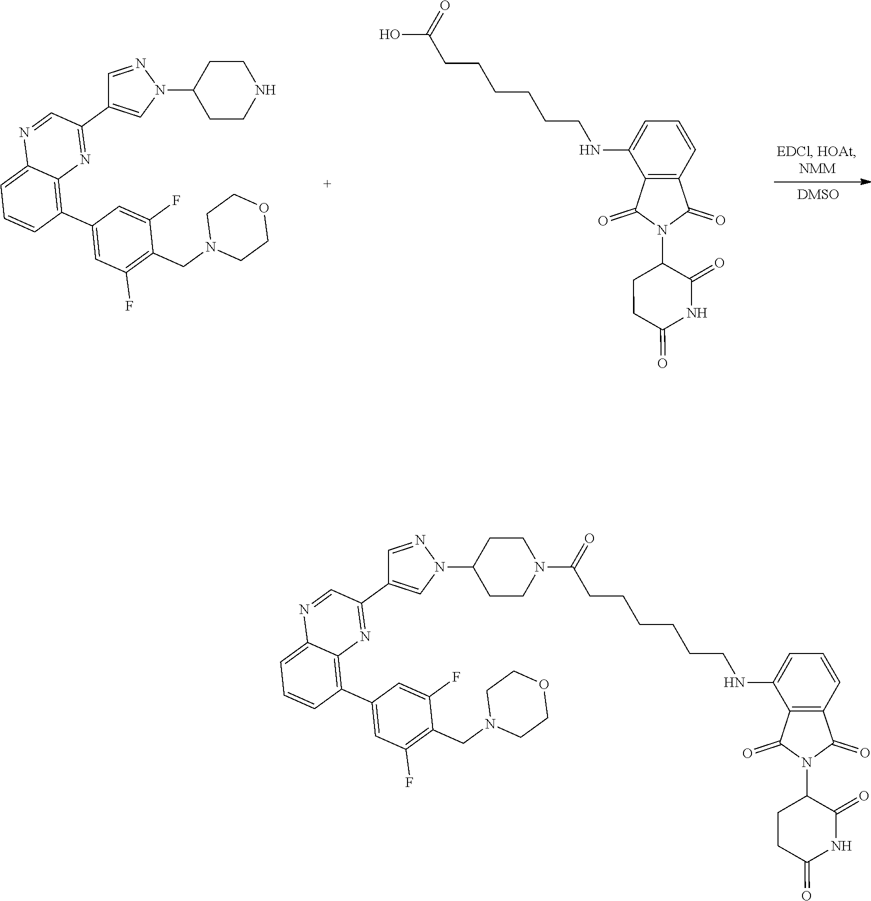 [SMILES: Fc1cc(-c2cccc3ncc(-c4cnn(C5CCNCC5)c4)nc23)cc(F)c1CN1CCOCC1 ; "+" ; O=C(O)CCCCCCNc1cccc2c1C(=O)N(C1CCC(=O)NC1=O)C2=O ; O=C1CCC(N2C(=O)c3cccc(NCCCCCCC(=O)N4CCC(n5cc(-c6cnc7cccc(-c8cc(F)c(CN9CCOCC9)c(F)c8)c7n6)cn5)CC4)c3C2=O)C(=O)N1]

JA-040 was synthesized following the standard procedure for preparing JA-001 (8.1 mg, 56.8% yield). MS (ESI) m/z: 874.4 [M+H]⁺.

### Example 129. (2S,4R)-1-[(2S)-2-[[6-[4-[4-[8-[3,5-Difluoro-4-(morpholinomethyl)phenyl]quinoxalin-2-yl]pyrazol-1-yl]-1-piperidyl]-6-oxo-hexanoyl]amino]-3,3-dimethyl-butanoyl]-4-hydroxy-N-[[4-(4-methylthiazol-5-yl)phenyl]methyl]pyrrolidine-2-carboxamide (JA-041)

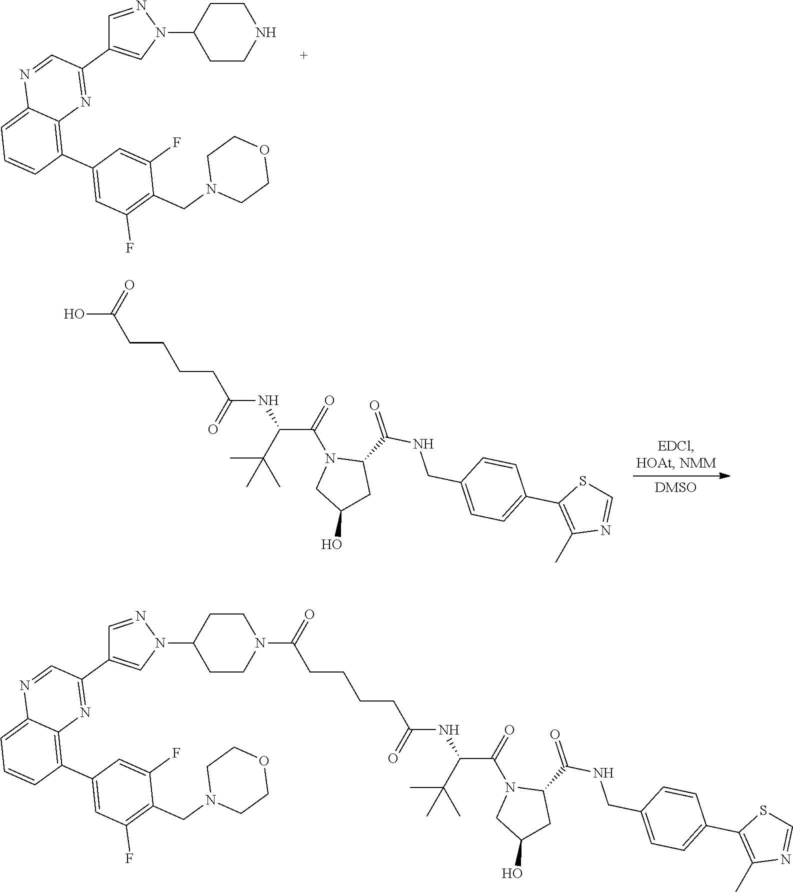

JA-041 was synthesized following the standard procedure for preparing JA-001 (6.7 mg, 39.8% yield). MS (ESI) m/z: 1031.5 [M+H]⁺.

### Example 130. (2S,4R)-1-[(2S)-2-[[10-[4-[4-[8-[3,5-Difluoro-4-(morpholinomethyl)phenyl]quinoxalin-2-yl]pyrazol-1-yl]-1-piperidyl]-10-oxo-decanoyl]amino]-3,3-dimethyl-butanoyl]-4-hydroxy-N-[[4-(4-methylthiazol-5-yl)phenyl]methyl]pyrrolidine-2-carboxamide (JA-042)

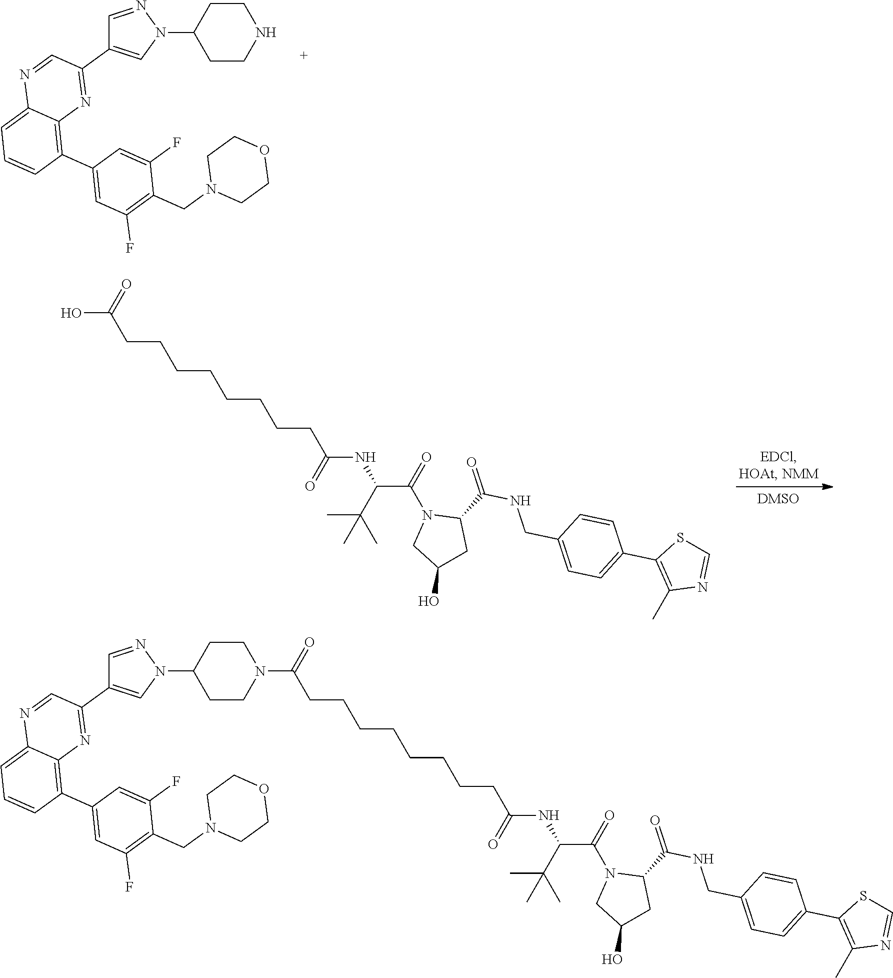

JA-042 was synthesized following the standard procedure for preparing JA-001 (5.9 mg, 33.2% yield). MS (ESI) m/z: 1087.5 [1M+H]⁺.

### Example 131. 4-[2-[2-[2-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethoxy]ethoxy]ethoxy]ethylamino]-2-(2,6-dioxo-3-piperidyl)isoindoline-1,3-dione (JA-043)

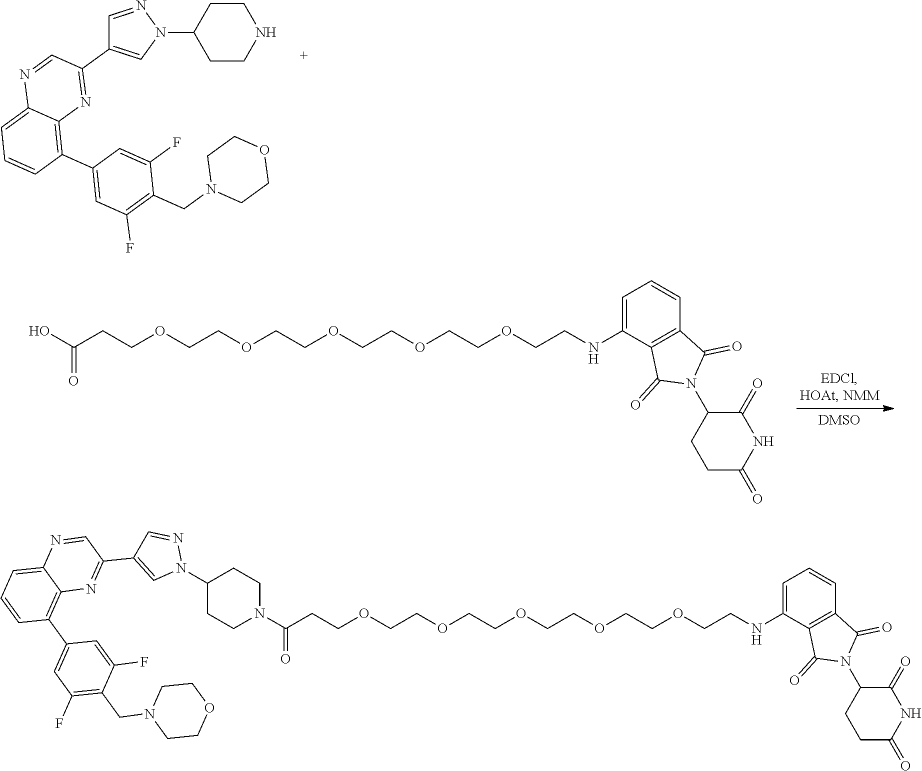

JA-043 was synthesized following the standard procedure for preparing JA-001 (7.8 mg, 46.1% yield). MS (ESI) m/z: 1038.4 [M+H]⁺.

### Example 132. (2S,4R)-1-[(2S)-2-[3-[2-[2-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethoxy]ethoxy]ethoxy]propanoylamino]-3,3-dimethyl-butanoyl]-4-hydroxy-N-[[4-(4-methylthiazol-5-yl)phenyl]methyl]pyrrolidine-2-carboxamide (JA-044)

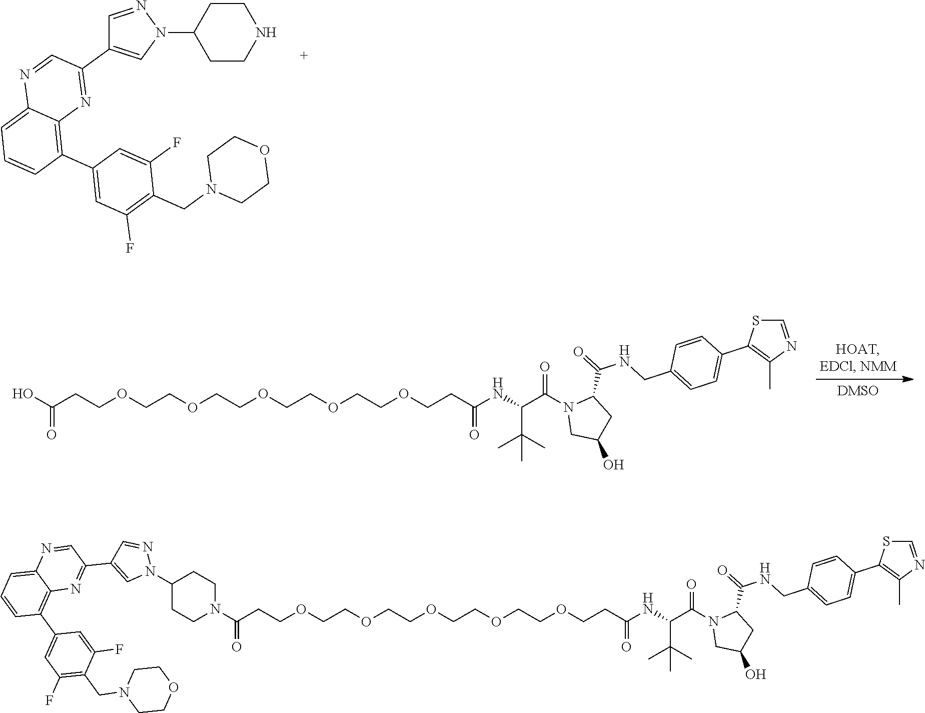

JA-044 was synthesized following the standard procedure for preparing JA-001 (9.1 mg, 45.6% yield). MS (ESI) m/z: 1223.6 [M+H]⁺.

### Example 133. 4-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethylamino]-2-(2,6-dioxo-3-piperidyl)isoindoline-1,3-dione (JA-045)

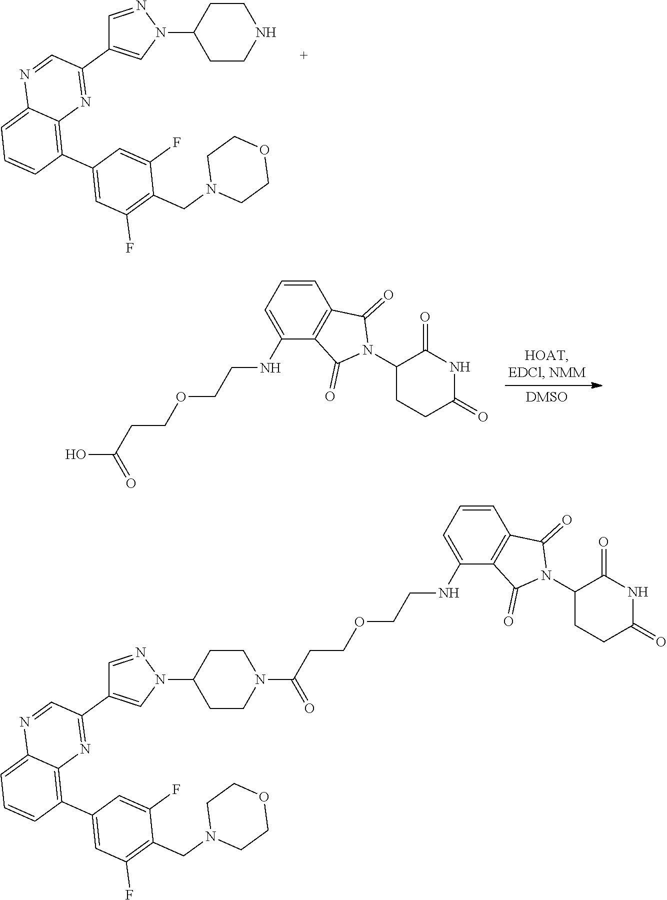

JA-045 was synthesized following the standard procedure for preparing JA-001 (6.8 mg, 48.4% yield). MS (ESI) m/z: 862.3 [M+H]⁺.

### Example 134. (2S,4R)-1-[(2S)-2-[[5-[4-[4-[8-[3,5-Difluoro-4-(morpholinomethyl)phenyl]quinoxalin-2-yl]pyrazol-1-yl]-1-piperidyl]-5-oxo-pentanoyl]amino]-3,3-dimethyl-butanoyl]-4-hydroxy-N-[[4-(4-methylthiazol-5-yl)phenyl]methyl]pyrrolidine-2-carboxamide (JA-046)

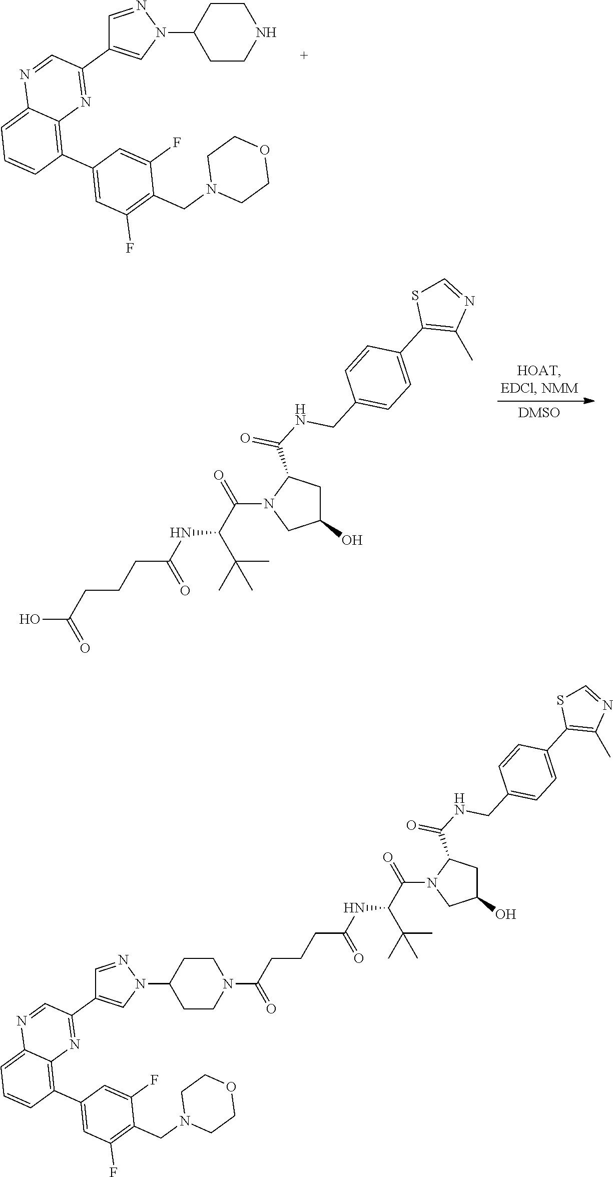

JA-046 was synthesized following the standard procedure for preparing JA-001 (7.6 mg, 45.8% yield). MS (ESI) m/z: 1017.5 [M+H]⁺.

### Example 135. 4-[2-[2-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethoxy]ethoxy]ethylamino]-2-(2,6-dioxo-3-piperidyl)isoindoline-1,3-dione (JA-047)

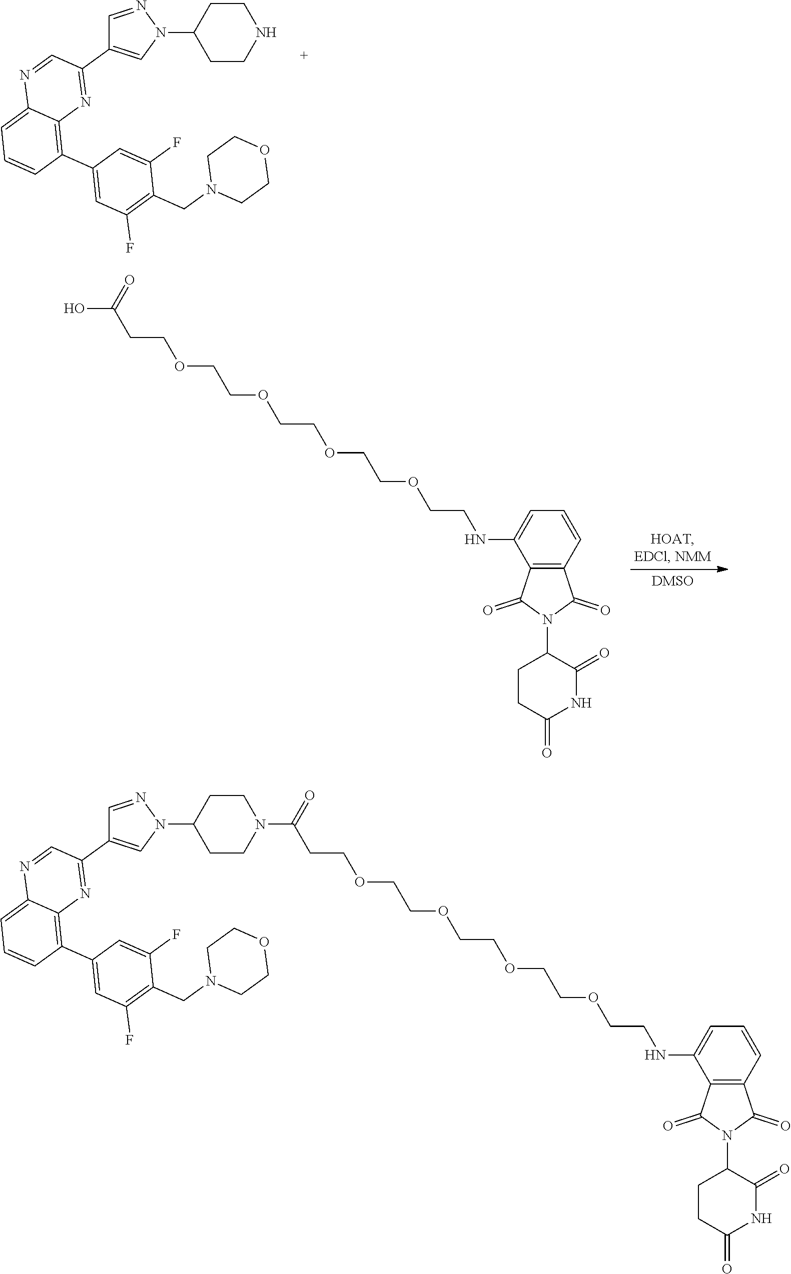

JA-047 was synthesized following the standard procedure for preparing JA-001 (8.2 mg, 50.5% yield). MS (ESI) m/z: 994.4 [M+H]⁺.

### Example 136. (2S,4R)-1-[(2S)-2-[[7-[4-[4-[8-[3,5-Difluoro-4-(morpholinomethyl)phenyl]quinoxalin-2-yl]pyrazol-1-yl]-1-piperidyl]-7-oxo-heptanoyl]amino]-3,3-dimethyl-butanoyl]-4-hydroxy-N-[[4-(4-methylthiazol-5-yl)phenyl]methyl]pyrrolidine-2-carboxamide (JA-048)

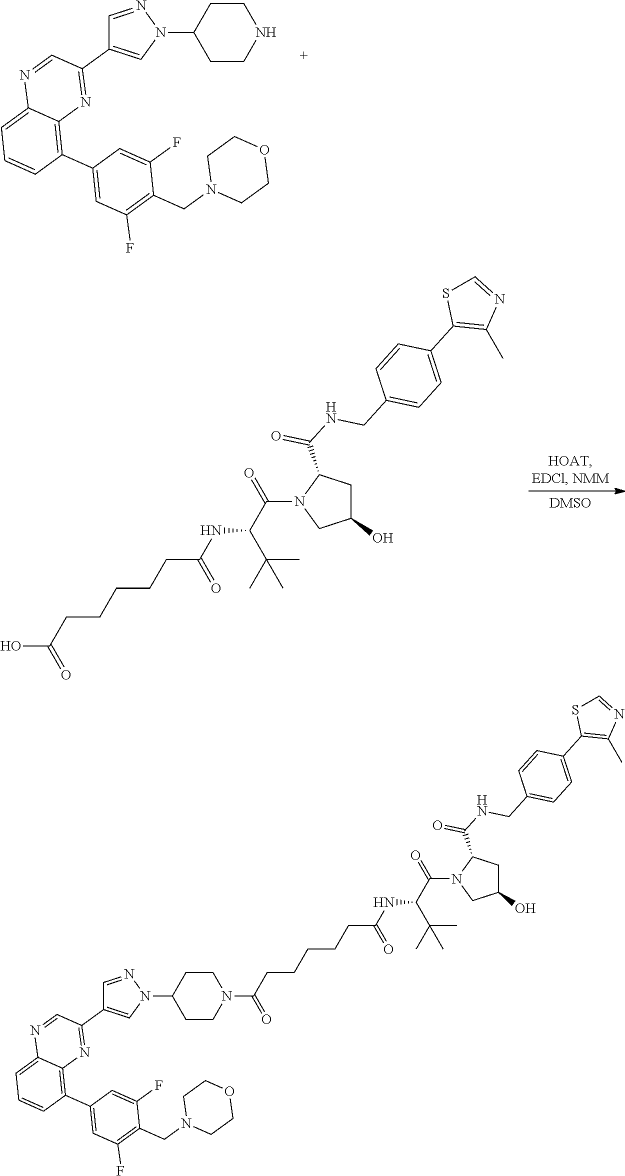

JA-048 was synthesized following the standard procedure for preparing JA-001 (9.0 mg, 52.8% yield). MS (ESI) m/z: 1045.5 [M+H]⁺.

### Example 137. 4-[2-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethoxy]ethylamino]-2-(2,6-dioxo-3-piperidyl)isoindoline-1,3-dione (JA-049)

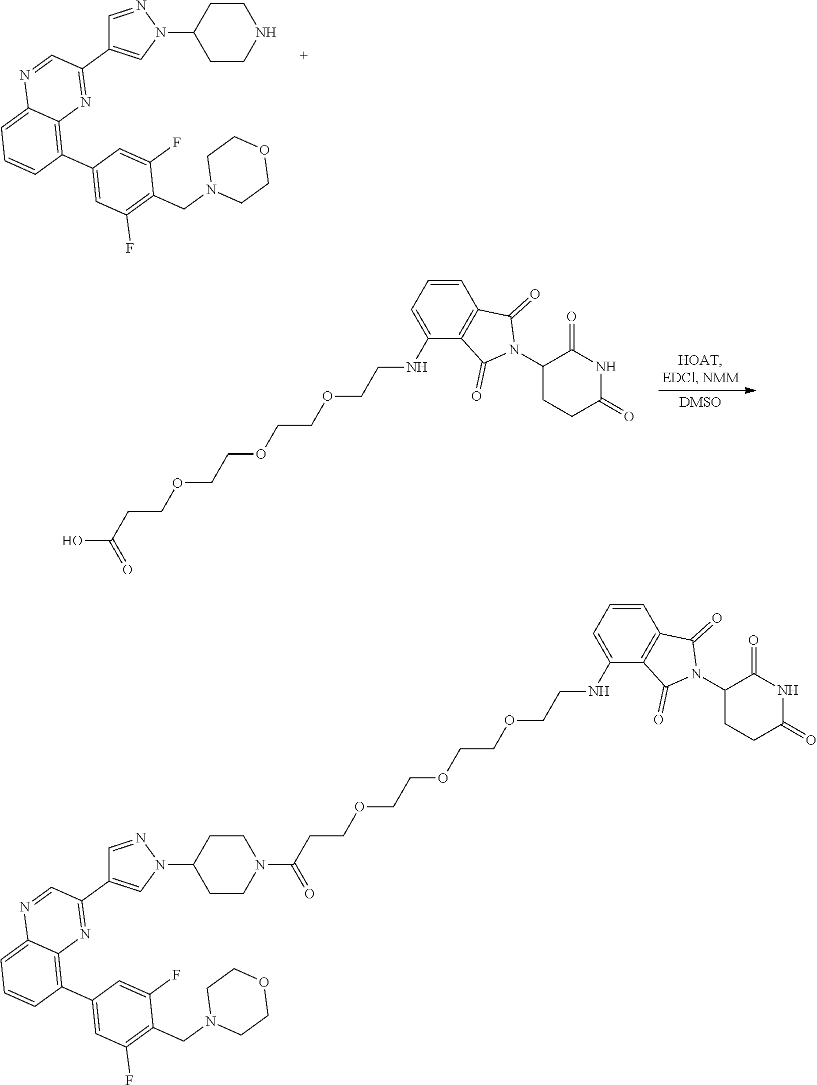

JA-049 was synthesized following the standard procedure for preparing JA-001 (7.8 mg, 50.3% yield). MS (ESI) m/z: 950.4 [M+H]⁺.

### Example 138. (2S,4R)-1-[(2S)-2-[3-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethoxy]propanoylamino]-3,3-dimethyl-butanoyl]-4-hydroxy-N-[[4-(4-methylthiazol-5-yl)phenyl]methyl]pyrrolidine-2-carboxamide (JA-050)

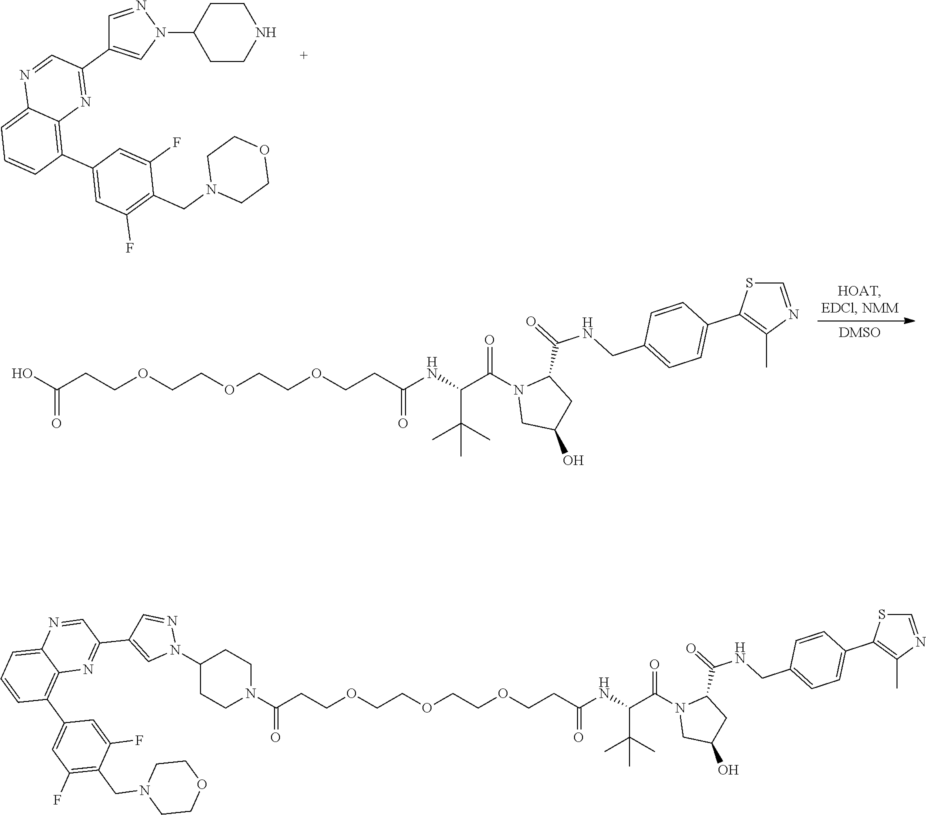

JA-050 was synthesized following the standard procedure for preparing JA-001 (8.3 mg, 44.8% yield). MS (ESI) m/z: 1135.5 [M+H]⁺.

### Example 139. (2S,4R)-1-[(2S)-2-[[9-[4-[4-[8-[3,5-Difluoro-4-(morpholinomethyl)phenyl]quinoxalin-2-yl]pyrazol-1-yl]-1-piperidyl]-9-oxo-nonanoyl]amino]-3,3-dimethyl-butanoyl]-4-hydroxy-N-[[4-(4-methylthiazol-5-yl)phenyl]methyl]pyrrolidine-2-carboxamide (JA-051)

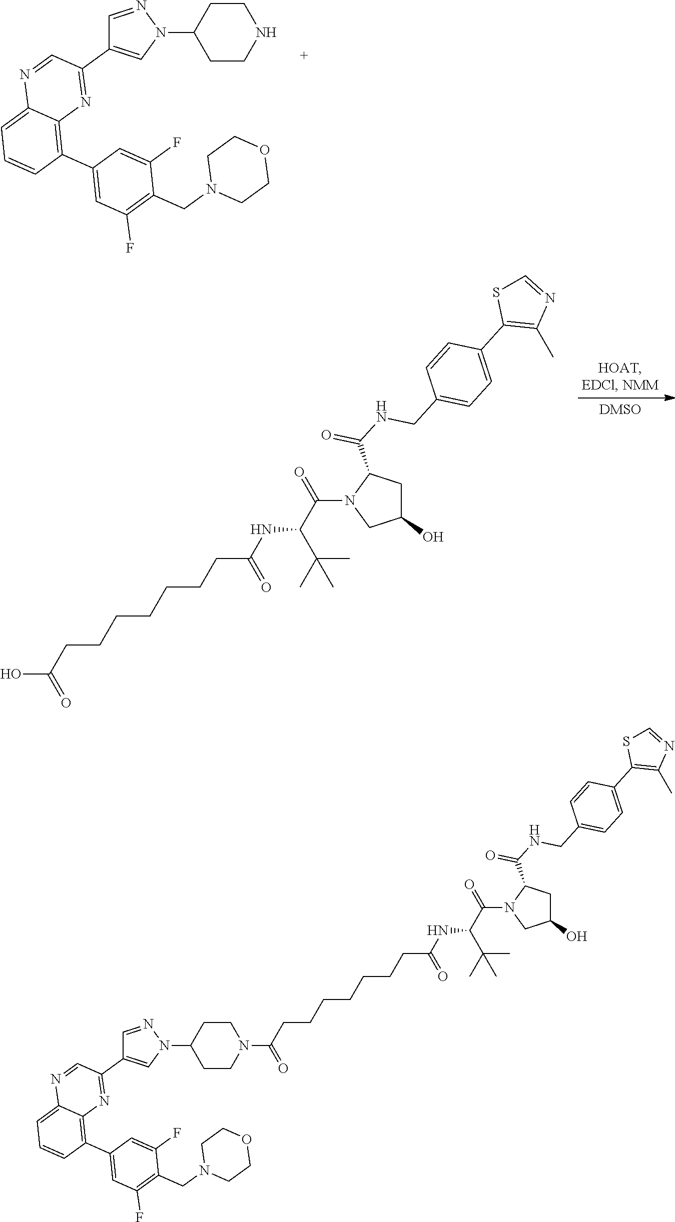

JA-051 was synthesized following the standard procedure for preparing JA-001 (8.0 mg, 45.7% yield). MS (ESI) m/z: 1073.5 [1M+H]⁺.

### Example 140. (2S,4R)-1-[1 (2S)-2-[[8-[4-[4-[8-[3,5-Difluoro-4-(morpholinomethyl)phenyl]quinoxalin-2-yl]pyrazol-1-yl]-1-piperidyl]-8-oxo-octanoyl]amino]-3,3-dimethyl-butanoyl]-4-hydroxy-N-[[14-(4-methylthiazol-5-yl)phenyl]methyl]pyrrolidine-2-carboxamide (JA-052)

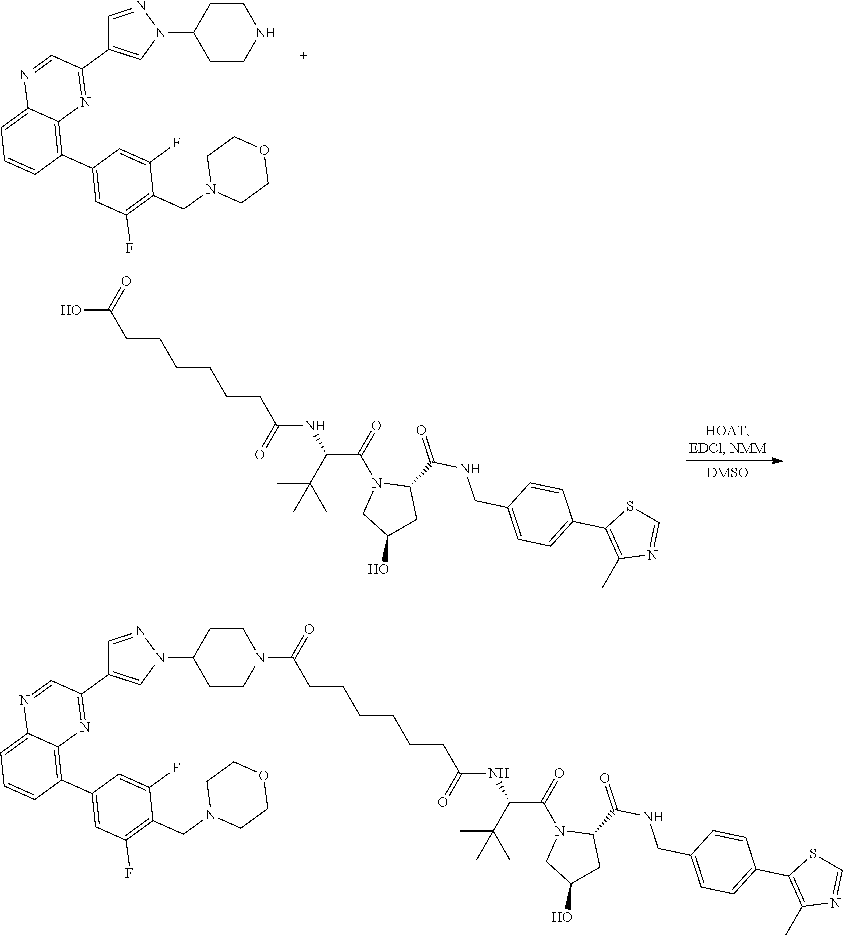

JA-052 was synthesized following the standard procedure for preparing JA-001 (7.3 mg, 42.2% yield). MS (ESI) m/z: 1059.5 [M+H]⁺.

### Example 141. (2S,4R)-1-[(2S)-2-[[2-[2-[2-[2-[4-[4-[8-[3,5-Difluoro-4-(morpholinomethyl)phenyl]quinoxalin-2-yl]pyrazol-1-yl]-1-piperidyl]-2-oxo-ethoxy]ethoxy]ethoxy]acetyl]amino]-3,3-dimethyl-butanoyl]-4-hydroxy-N-[[4-(4-methylthiazol-5-yl)phenyl]methyl]pyrrolidine-2-carboxamide (JA-053)

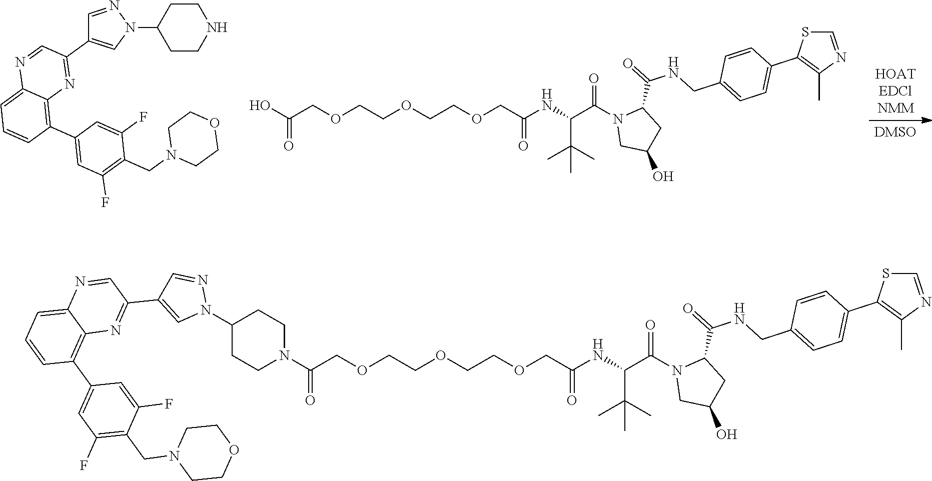

JA-053 was synthesized following the standard procedure for preparing JA-001 (8.9 mg, 49.3% yield). MS (ESI) m/z: 1107.5 [M+H]⁺.

### Example 142. (2S,4R)-1-[(2S)-2-[[2-[2-[2-[4-[4-[8-[3,5-Difluoro-4-(morpholinomethyl)phenyl]quinoxalin-2-yl]pyrazol-1-yl]-1-piperidyl]-2-oxo-ethoxy]ethoxy]acetyl]amino]-3,3-dimethyl-butanoyl]-4-hydroxy-N-[[4-(4-methylthiazol-5-yl)phenyl]methyl]pyrrolidine-2-carboxamide (JA-054)

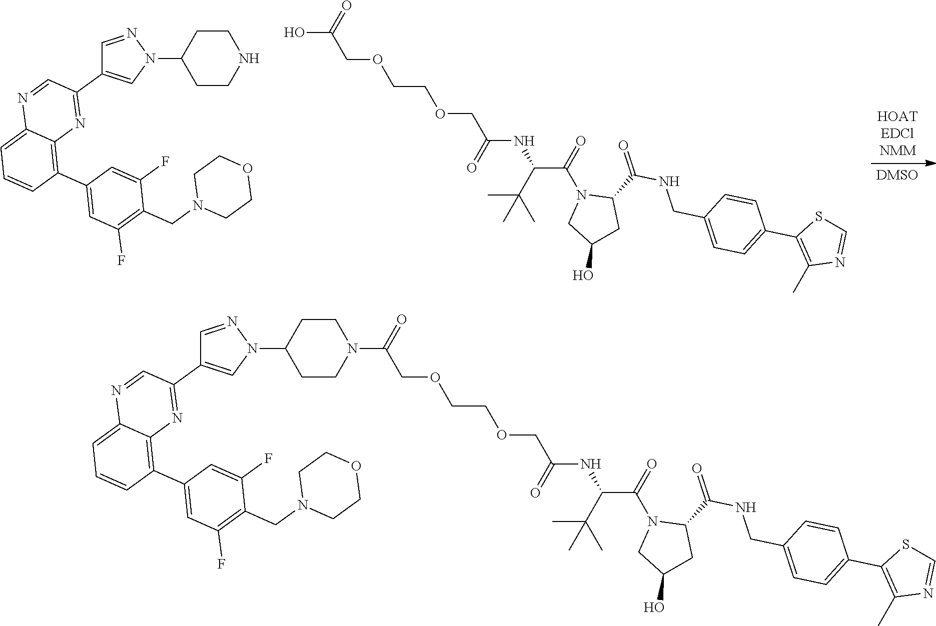

JA-054 was synthesized following the standard procedure for preparing JA-001 (8.3 mg, 47.8% yield). MS (ESI) m/z: 1063.5 [M+H]⁺.

### Example 143. (2S,4R)-1-[(2S)-2-[[2-[2-[2-[2-[2-[2-[4-[4-[8-[3,5-Difluoro-4-(morpholinomethyl)phenyl]quinoxalin-2-yl]pyrazol-1-yl]-1-piperidyl]-2-oxo-ethoxy]ethoxy]ethoxy]ethoxy]ethoxy]acetyl]amino]-3,3-dimethyl-butanoyl]-4-hydroxy-N-[[4-(4-methylthiazol-5-yl)phenyl]methyl]pyrrolidine-2-carboxamide (JA-055)

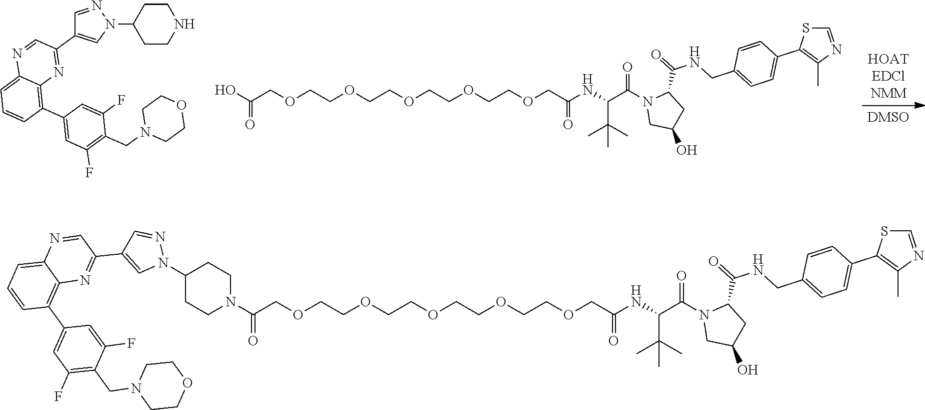

JA-055 was synthesized following the standard procedure for preparing JA-001 (6.9 mg, 35.4% yield). MS (ESI) m/z: 1195.5 [M+H]⁺.

### Example 144. (2S,4R)-1-[(2S)-2-[3-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]propanoylamino]-3,3-dimethyl-butanoyl]-4-hydroxy-N-[[4-(4-methylthiazol-5-yl)phenyl]methyl]pyrrolidine-2-carboxamide (JA-056)

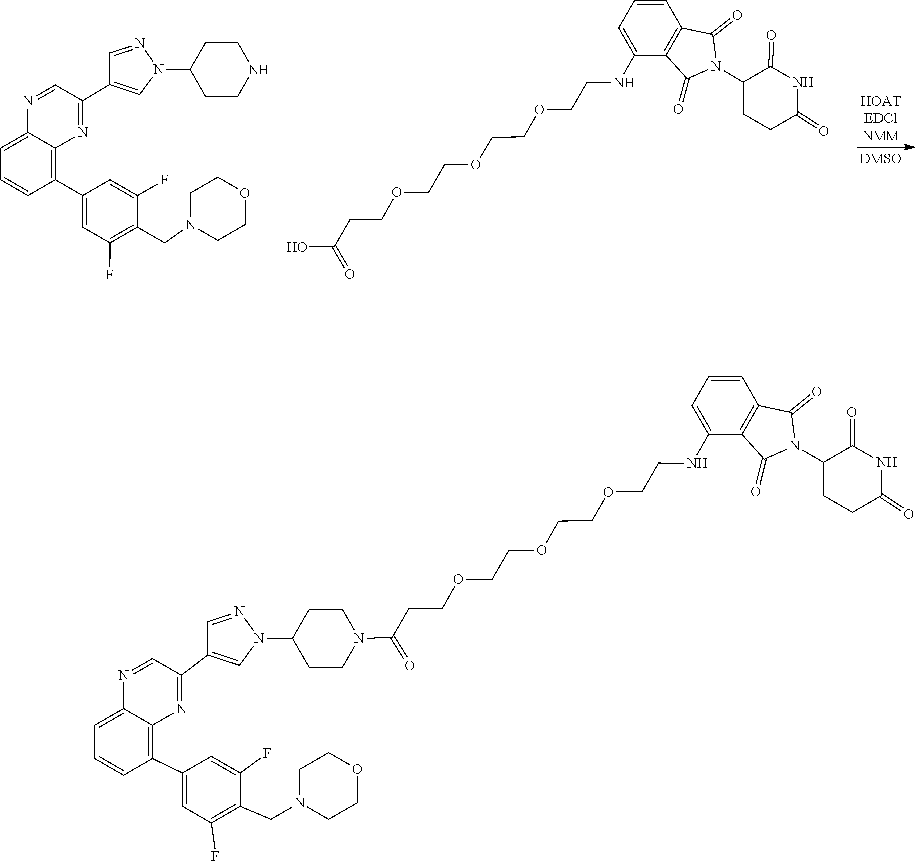 [SMILES: Fc1cc(-c2cccc3ncc(-c4cnn(C5CCNCC5)c4)nc23)cc(F)c1CN1CCOCC1 ; O=C(O)CCOCCOCCOCCNc1cccc2c1C(=O)N(C1CCC(=O)NC1=O)C2=O ; O=C1CCC(N2C(=O)c3cccc(NCCOCCOCCOCCC(=O)N4CCC(n5cc(-c6cnc7cccc(-c8cc(F)c(CN9CCOCC9)c(F)c8)c7n6)cn5)CC4)c3C2=O)C(=O)N1]

JA-056 was synthesized following the standard procedure for preparing JA-001 (6.9 mg, 38.7% yield). MS (ESI) m/z: 1091.5 [1M+H]⁺.

### Example 145. (2S,4R)-1-[(2S)-2-[[2-[2-[4-[4-[8-[3,5-Difluoro-4-(morpholinomethyl)phenyl]quinoxalin-2-yl]pyrazol-1-yl]-1-piperidyl]-2-oxo-ethoxy]acetyl]amino]-3,3-dimethyl-butanoyl]-4-hydroxy-N-[[4-(4-methylthiazol-5-yl)phenyl]methyl]pyrrolidine-2-carboxamide (JA-057)

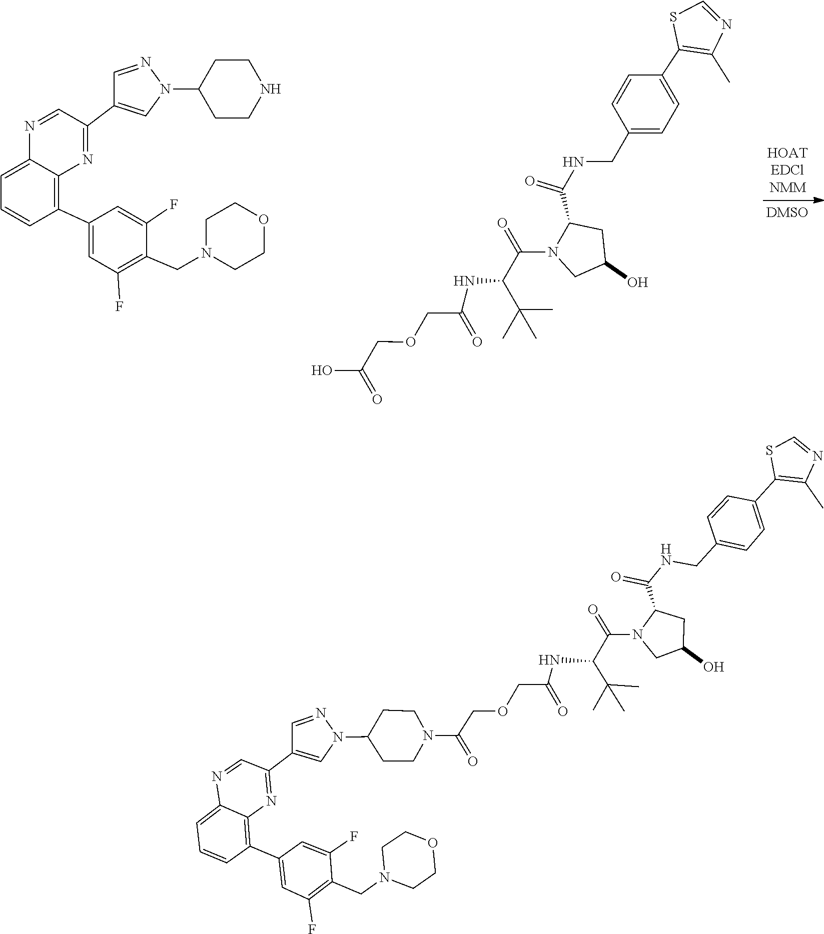 [SMILES: Fc1cc(-c2cccc3ncc(-c4cnn(C5CCNCC5)c4)nc23)cc(F)c1CN1CCOCC1 ; Cc1ncsc1-c1ccc(CNC(=O)[C@@H]2C[C@@H](O)CN2C(=O)[C@@H](NC(=O)COCC(=O)O)C(C)(C)C)cc1 ; Cc1ncsc1-c1ccc(CNC(=O)[C@@H]2C[C@@H](O)CN2C(=O)[C@@H](NC(=O)COCC(=O)N2CCC(n3cc(-c4cnc5cccc(-c6cc(F)c(CN7CCOCC7)c(F)c6)c5n4)cn3)CC2)C(C)(C)C)cc1]

JA-057 was synthesized following the standard procedure for preparing JA-001 (9.1 mg, 54.7% yield). MS (ESI) m/z: 1019.4 [M+H]⁺.

### Example 146. (2S,4R)-1-[(2S)-2-[3-[3-[4-[4-[8-[3,5-Difluoro-4-(morpholinomethyl)phenyl]quinoxalin-2-yl]pyrazol-1-yl]-1-piperidyl]-3-oxo-propoxy]propanoylamino]-3,3-dimethyl-butanoyl]-4-hydroxy-N-[[4-(4-methylthiazol-5-yl)phenyl]methyl]pyrrolidine-2-carboxamide (JA-058)

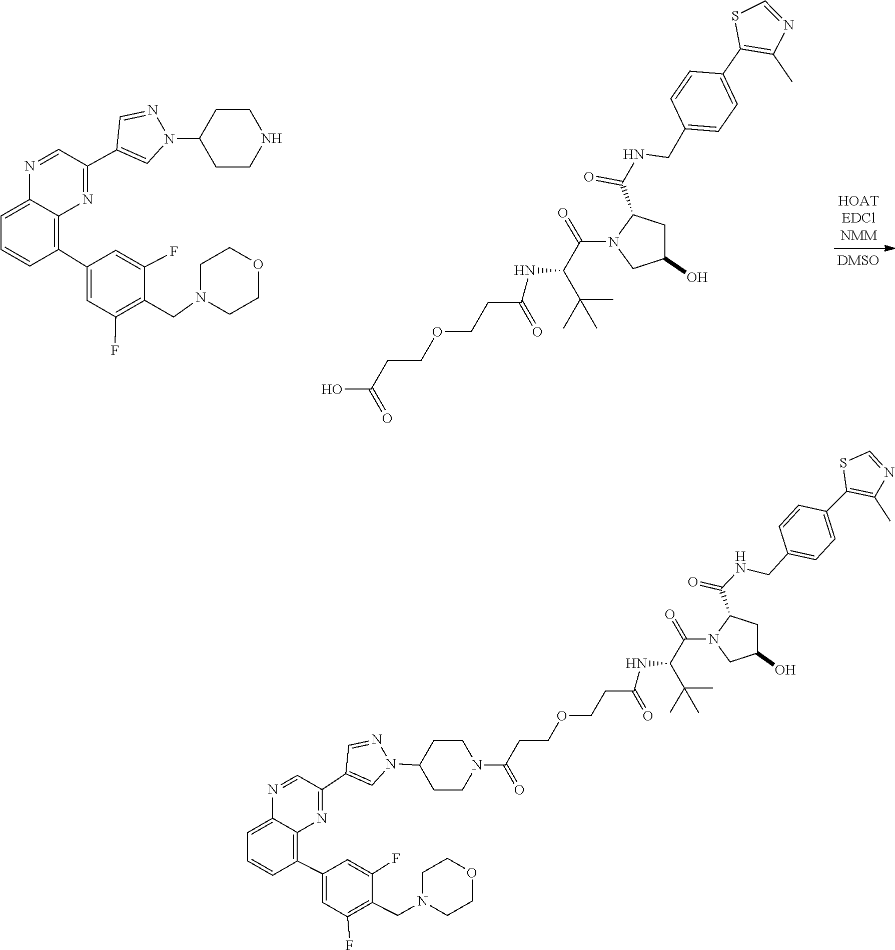 [SMILES: Fc1cc(-c2cccc3ncc(-c4cnn(C5CCNCC5)c4)nc23)cc(F)c1CN1CCOCC1 ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)N2CCC(n3cc(-c4cnc5cccc(-c6cc(F)c(CN7CCOCC7)c(F)c6)c5n4)cn3)CC2)C(C)(C)C)cc1]

JA-058 was synthesized following the standard procedure for preparing JA-001 (9.7 mg, 56.8% yield). MS (ESI) m/z: 1047.5 [M+H]⁺.

### Example 147. (2S,4R)-1-[(2S)-2-[[11-[4-[4-[8-[3,5-Difluoro-4-(morpholinomethyl)phenyl]quinoxalin-2-yl]pyrazol-1-yl]-1-piperidyl]-11-oxo-undecanoyl]amino]-3,3-dimethyl-butanoyl]-4-hydroxy-N-[[4-(4-methylthiazol-5-yl)phenyl]methyl]pyrrolidine-2-carboxamide (JA-059)

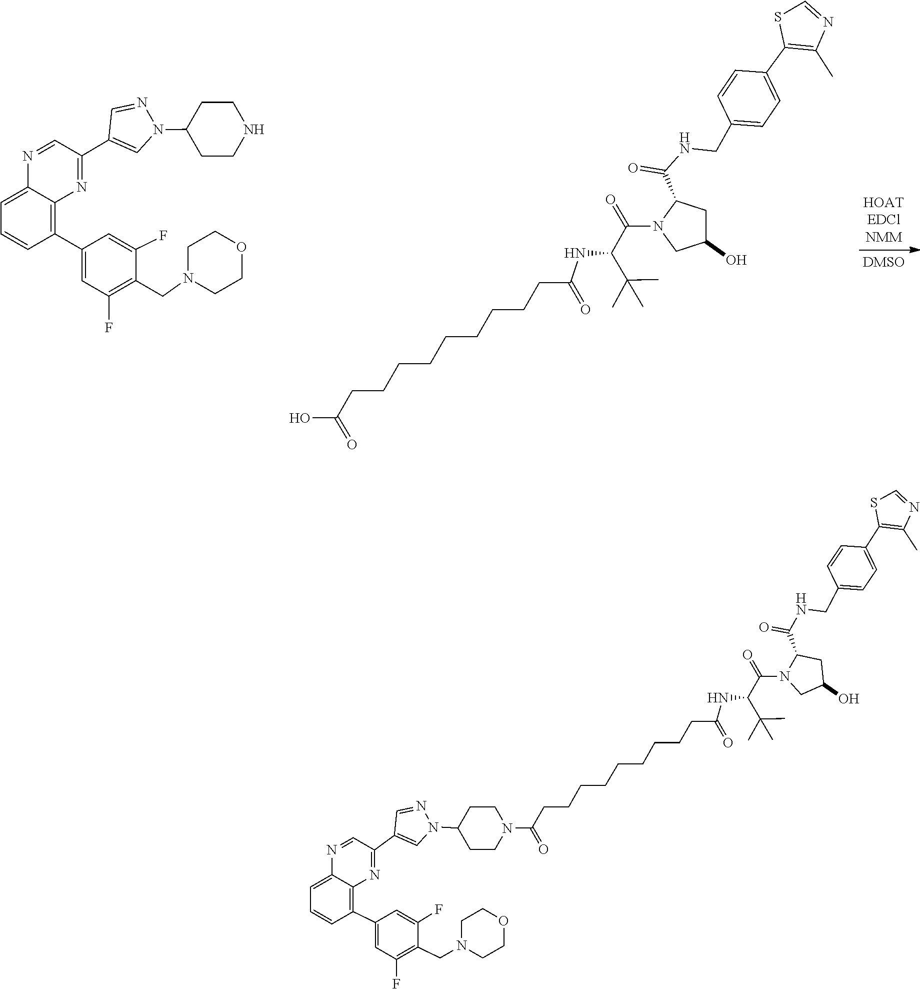 [SMILES: Fc1cc(-c2cccc3ncc(-c4cnn(C5CCNCC5)c4)nc23)cc(F)c1CN1CCOCC1 ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCCC(=O)N2CCC(n3cc(-c4cnc5cccc(-c6cc(F)c(CN7CCOCC7)c(F)c6)c5n4)cn3)CC2)C(C)(C)C)cc1]

JA-059 was synthesized following the standard procedure for preparing JA-001 (9.0 mg, 50.1% yield). MS (ESI) m/z: 1101.5 [1M+H]⁺.

### Example 148. (2S,4R)-1-[(2S)-2-[[4-[4-[4-[8-[3,5-Difluoro-4-(morpholinomethyl)phenyl]quinoxalin-2-yl]pyrazol-1-yl]-1-piperidyl]-4-oxo-butanoyl]amino]-3,3-dimethyl-butanoyl]-4-hydroxy-N-[[4-(4-methylthiazol-5-yl)phenyl]methyl]pyrrolidine-2-carboxamide (JA-060)

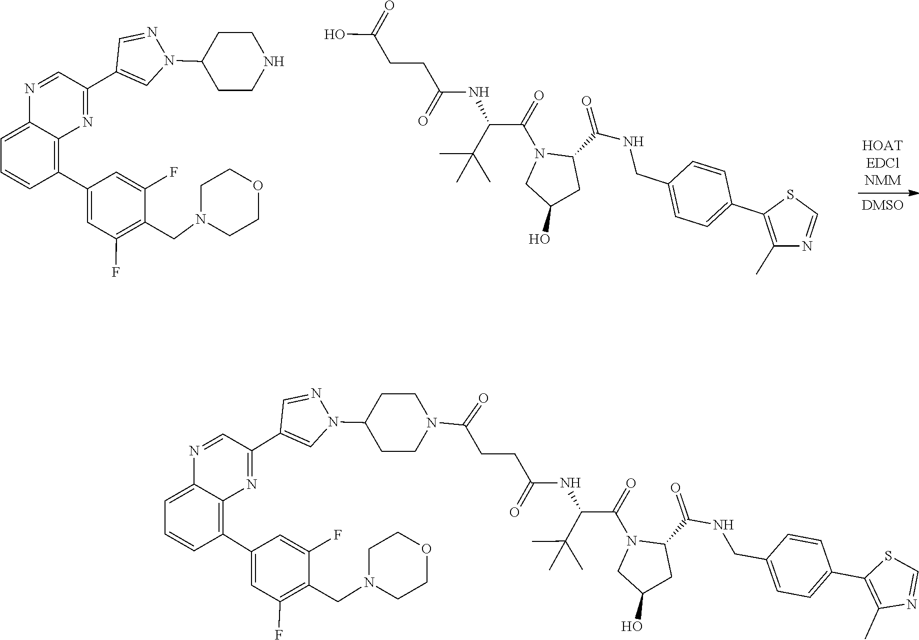 [SMILES: Fc1cc(-c2cccc3ncc(-c4cnn(C5CCNCC5)c4)nc23)cc(F)c1CN1CCOCC1 ; Cc1ncsc1-c1ccc(CNC(=O)[C@@H]2C[C@@H](O)CN2C(=O)[C@@H](NC(=O)CCC(=O)O)C(C)(C)C)cc1 ; Cc1ncsc1-c1ccc(CNC(=O)[C@@H]2C[C@@H](O)CN2C(=O)[C@@H](NC(=O)CCC(=O)N2CCC(n3cc(-c4cnc5cccc(-c6cc(F)c(CN7CCOCC7)c(F)c6)c5n4)cn3)CC2)C(C)(C)C)cc1]

JA-060 was synthesized following the standard procedure for preparing JA-001 (8.9 mg, 54.4% yield). MS (ESI) m/z: 1003.4 [M+H]⁺.

### Example 149. (2S,4R)-1-[(2S)-2-[3-[2-[2-[2-[3-[4-[4-[8-[3,5-Difluoro-4-(morpholinomethyl)phenyl]quinoxalin-2-yl]pyrazol-1-yl]-1-piperidyl]-3-oxo-propoxy]ethoxy]ethoxy]ethoxy]propanoylamino]-3,3-dimethyl-butanoyl]-4-hydroxy-N-[[4-(4-methylthiazol-5-yl)phenyl]methyl]pyrrolidine-2-carboxamide (JA-061)

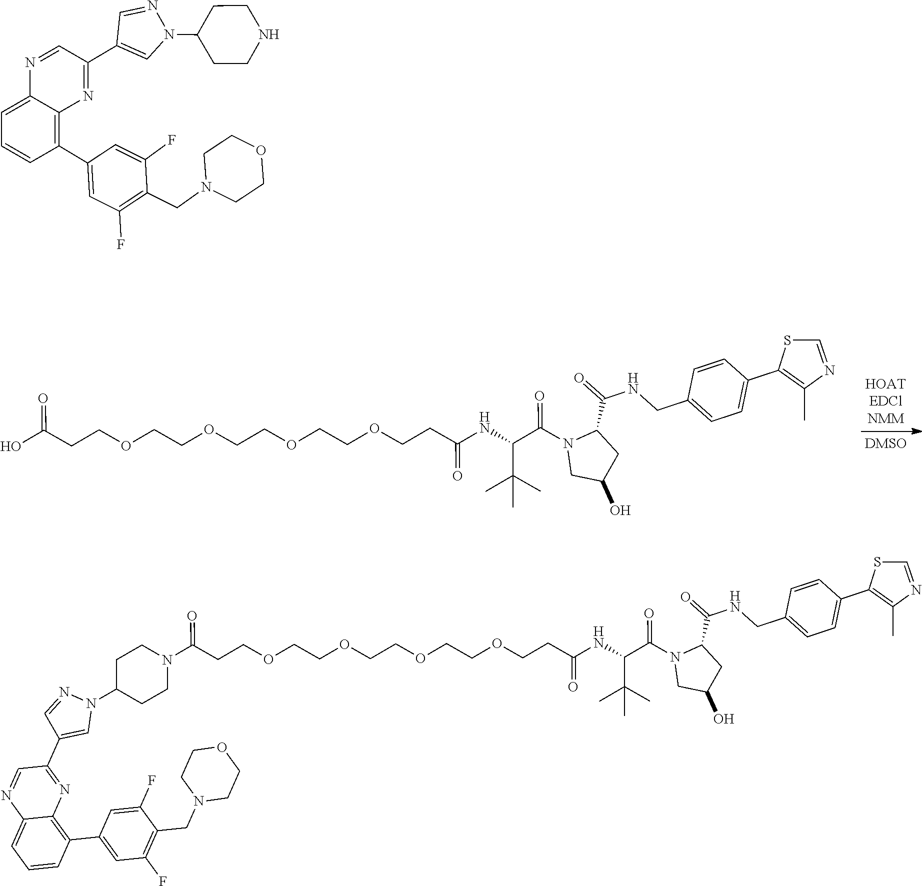 [SMILES: Fc1cc(-c2cccc3ncc(-c4cnn(C5CCNCC5)c4)nc23)cc(F)c1CN1CCOCC1 ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCC(=O)N2CCC(n3cc(-c4cnc5cccc(-c6cc(F)c(CN7CCOCC7)c(F)c6)c5n4)cn3)CC2)C(C)(C)C)cc1]

JA-061 was synthesized following the standard procedure for preparing JA-001 (10.8 mg, 56.1% yield). MS (ESI) m/z: 1179.5 [M+H]⁺.

### Example 150. (2S,4R)-1-((S)-3,3-Dimethyl-2-(8-(4-(4-((5-(4-(methylsulfonyl)phenyl)-[1,2,4]triazolo[1,5-a]pyridin-2-yl)amino)phenyl)piperazin-1-yl)-8-oxooctanamido)butanoyl)-4-hydroxy-N-(4-(4-methylthiazol-5-yl)benzyl)pyrrolidine-2-carboxamide (JA-062)

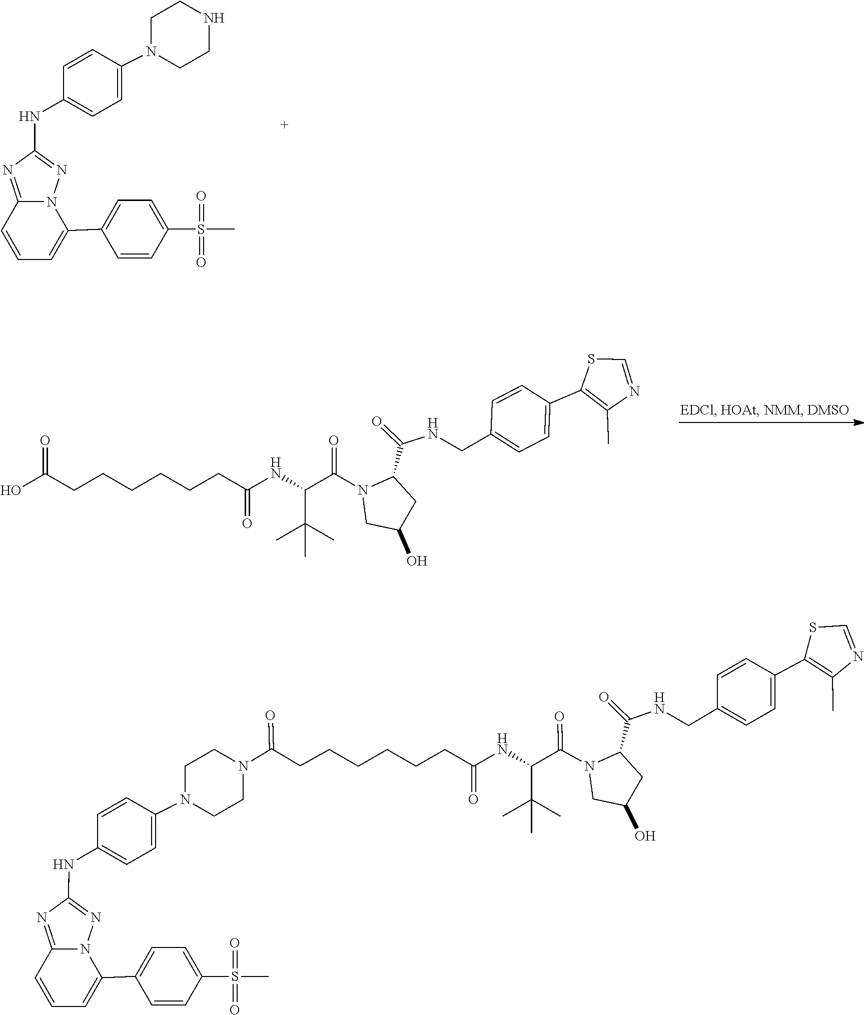 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CN2C(=O)[C@@H](NC(=O)CCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCC(=O)N2CCN(c3ccc(Nc4nc5cccc(-c6ccc(S(C)(=O)=O)cc6)n5n4)cc3)CC2)C(C)(C)C)cc1]

JA-062 was synthesized following the standard procedure for preparing JA-010 (7 mg, 60% yield). MS (ESI) m/z: 1017.4 [M+H]⁺.

### Example 151. (2S,4R)-1-((S)-2-(tert-Butyl)-19-(4-(4-((5-(4-(methylsulfonyl)phenyl)-[1,2,4]triazolo[1,5-a]pyridin-2-yl)amino)phenyl)piperazin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide (JA-063)

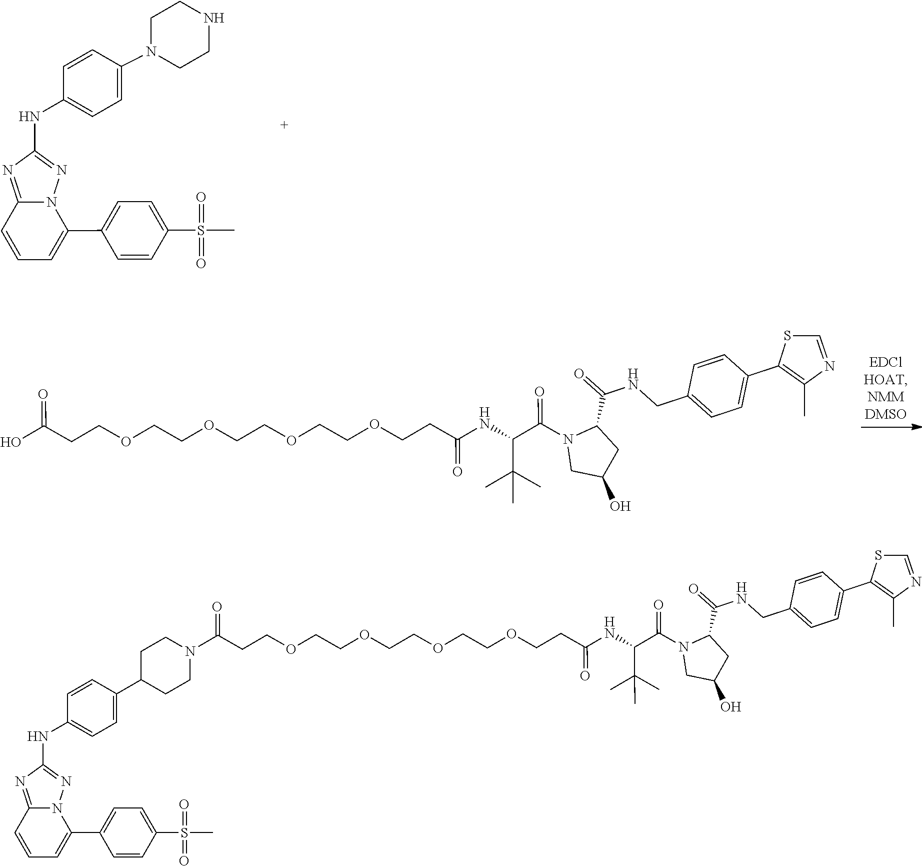 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCC(=O)N2CCC(c3ccc(Nc4nc5cccc(-c6ccc(S(C)(=O)=O)cc6)n5n4)cc3)CC2)C(C)(C)C)cc1]

JA-063 was synthesized following the standard procedure for preparing JA-010 (6.9 mg, 61% yield). MS (ESI) m/z: 1137.5 [M+H]⁺.

### Example 152. (2S,4R)-1-((S)-2-(tert-Butyl)-14-(4-(4-((5-(4-(methylsulfonyl)phenyl)-[1,2,4]triazolo[1,5-a]pyridin-2-yl)amino)phenyl)piperazin-1-yl)-4,14-dioxo-6,9,12-trioxa-3-azatetradecanoyl)-4-hydroxy-N-(4-(4-methylthiazol-5-yl)benzyl)pyrrolidine-2-carboxamide (JA-064)

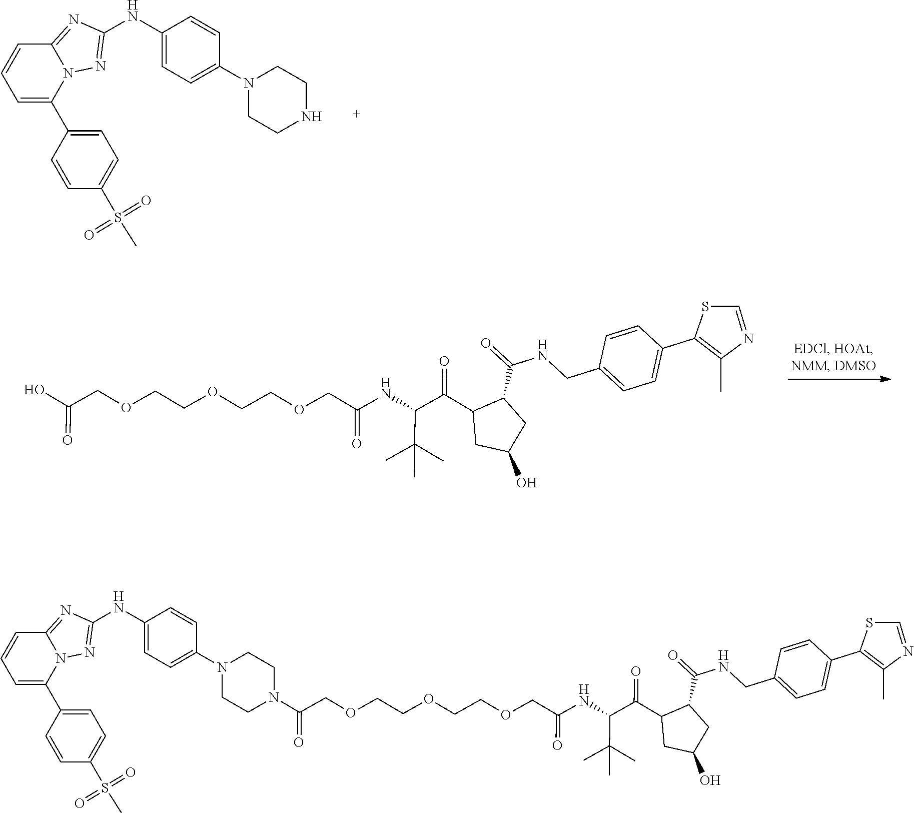 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CC2C(=O)[C@@H](NC(=O)COCCOCCOCC(=O)O)C(C)(C)C)cc1 ; Cc1ncsc1-c1ccc(CNC(=O)[C@@H]2C[C@@H](O)CC2C(=O)[C@@H](NC(=O)COCCOCCOCC(=O)N2CCN(c3ccc(Nc4nc5cccc(-c6ccc(S(C)(=O)=O)cc6)n5n4)cc3)CC2)C(C)(C)C)cc1]

JA-064 was synthesized following the standard procedure for preparing JA-010 (7.2 mg, 70% yield). MS (ESI) m/z: 1065.4 [M+H]⁺.

### Example 153. (2S,4R)-1-((S)-3,3-Dimethyl-2-(9-(4-(4-((5-(4-(methylsulfonyl)phenyl)-[1,2,4]triazolo[1,5-a]pyridin-2-yl)amino)phenyl)piperazin-1-yl)-9-oxononanamido)butanoyl)-4-hydroxy-N-(4-(4-methylthiazol-5-yl)benzyl)pyrrolidine-2-carboxamide (JA-065)

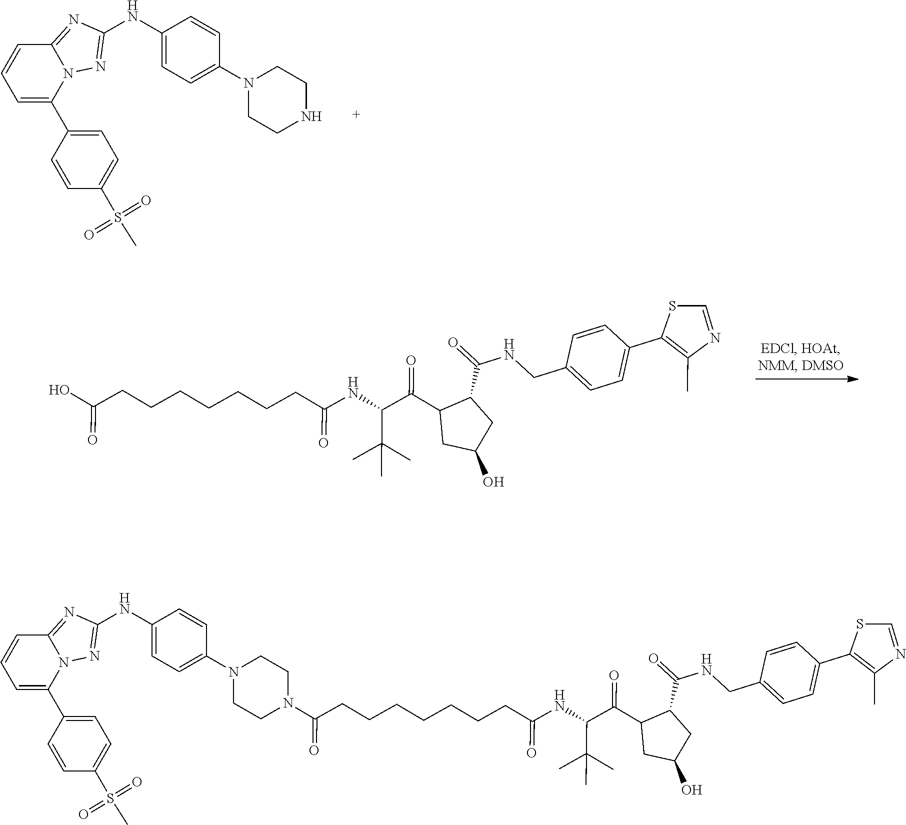 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CC2C(=O)[C@@H](NC(=O)CCCCCCCC(=O)O)C(C)(C)C)cc1 ; Cc1ncsc1-c1ccc(CNC(=O)[C@@H]2C[C@@H](O)CC2C(=O)[C@@H](NC(=O)CCCCCCCC(=O)N2CCN(c3ccc(Nc4nc5cccc(-c6ccc(S(C)(=O)=O)cc6)n5n4)cc3)CC2)C(C)(C)C)cc1]

JA-065 was synthesized following the standard procedure for preparing JA-010 (8.1 mg, 78% yield). MS (ESI) m/z: 1031.5 [M+H]⁺.

### Example 154. (2S,4R)-1-((S)-3,3-Dimethyl-2-(5-(4-(4-((5-(4-(methylsulfonyl)phenyl)-[1,2,4]triazolo[1,5-a]pyridin-2-yl)amino)phenyl)piperazin-1-yl)-5-oxopentanamido)butanoyl)-4-hydroxy-N-(4-(4-methylthiazol-5-yl)benzyl)pyrrolidine-2-carboxamide (JA-066)

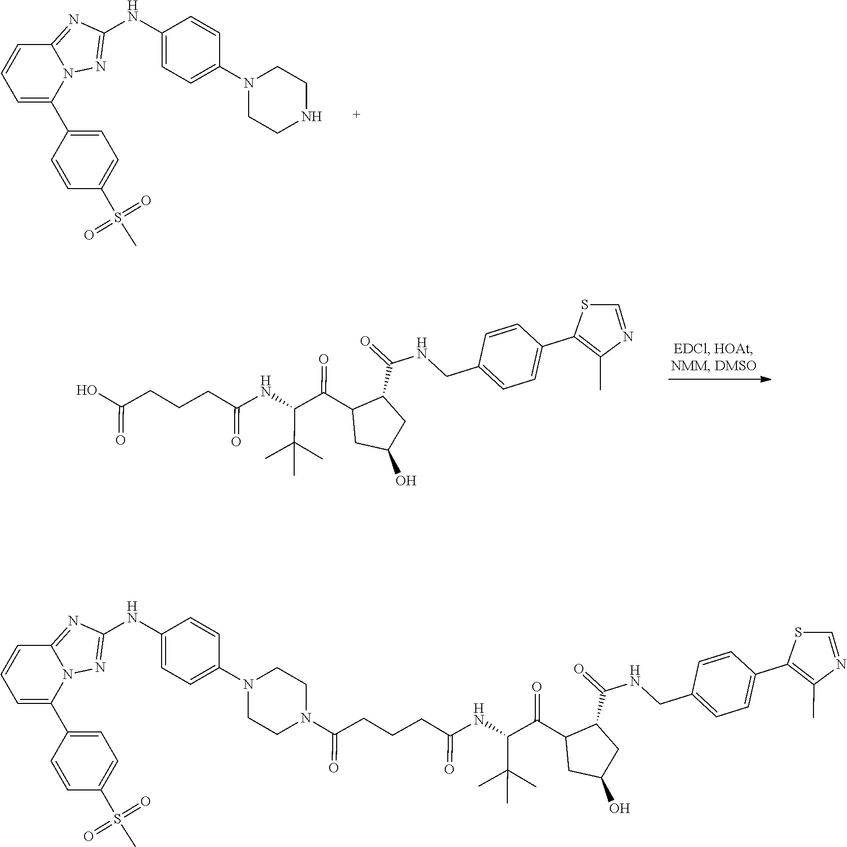 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CC2C(=O)[C@@H](NC(=O)CCCC(=O)O)C(C)(C)C)cc1 ; Cc1ncsc1-c1ccc(CNC(=O)[C@@H]2C[C@@H](O)CC2C(=O)[C@@H](NC(=O)CCCC(=O)N2CCN(c3ccc(Nc4nc5cccc(-c6ccc(S(C)(=O)=O)cc6)n5n4)cc3)CC2)C(C)(C)C)cc1]

JA-066 was synthesized following the standard procedure for preparing JA-010 (6 mg, 70% yield). MS (ESI) m/z: 975.4[M+H]⁺.

### Example 155. (2S,4R)-1-((S)-3,3-Dimethyl-2-(10-(4-(4-((5-(4-(methylsulfonyl)phenyl)-[1,2,4]triazolo[1,5-a]pyridin-2-yl)amino)phenyl)piperazin-1-yl)-10-oxodecanamido)butanoyl)-4-hydroxy-N-(4-(4-methylthiazol-5-yl)benzyl)pyrrolidine-2-carboxamide (JA-067)

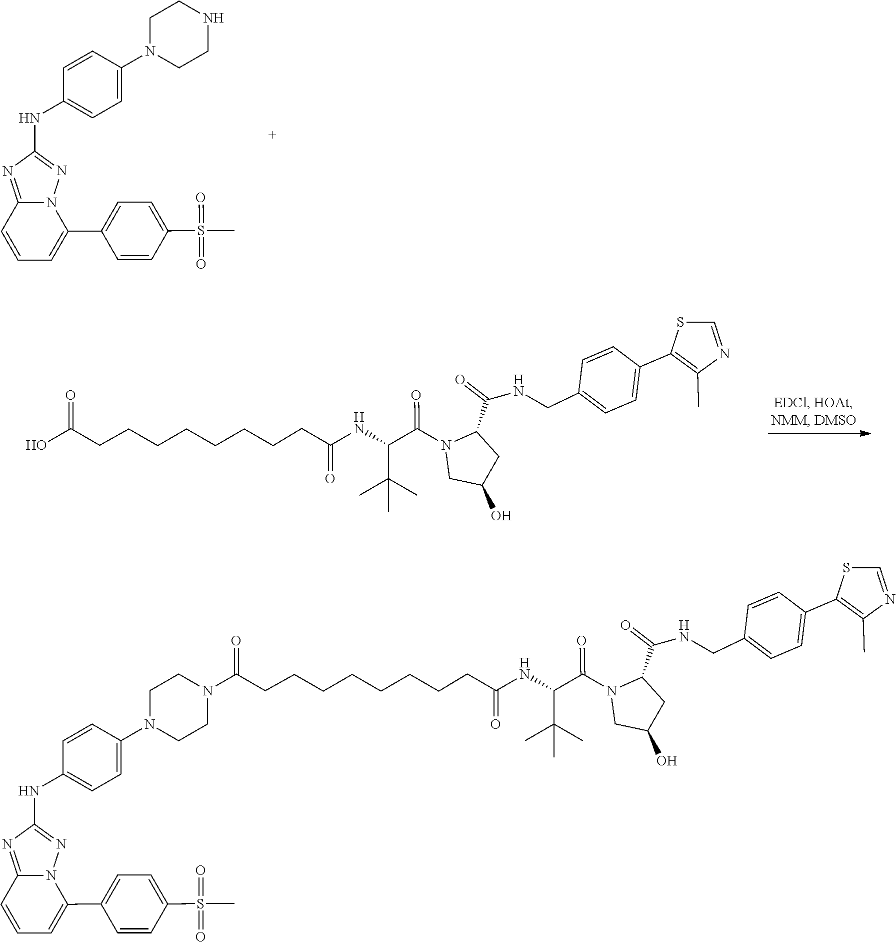 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CN2C(=O)[C@@H](NC(=O)CCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCC(=O)N2CCN(c3ccc(Nc4nc5cccc(-c6ccc(S(C)(=O)=O)cc6)n5n4)cc3)CC2)C(C)(C)C)cc1]

JA-067 was synthesized following the standard procedure for preparing JA-010 (7.2 mg, 66% yield). MS (ESI) m/z: 1045.4 [M+H]⁺.

### Example 156. (2S,4R)-1-((S)-2-(tert-Butyl)-16-(4-(4-((5-(4-(methylsulfonyl)phenyl)-[1,2,4]triazolo[1,5-a]pyridin-2-yl)amino)phenyl)piperazin-1-yl)-4,16-dioxo-7,10,13-trioxa-3-azahexadecanoyl)-4-hydroxy-N-(4-(4-methylthiazol-5-yl)benzyl)pyrrolidine-2-carboxamide (JA-068)

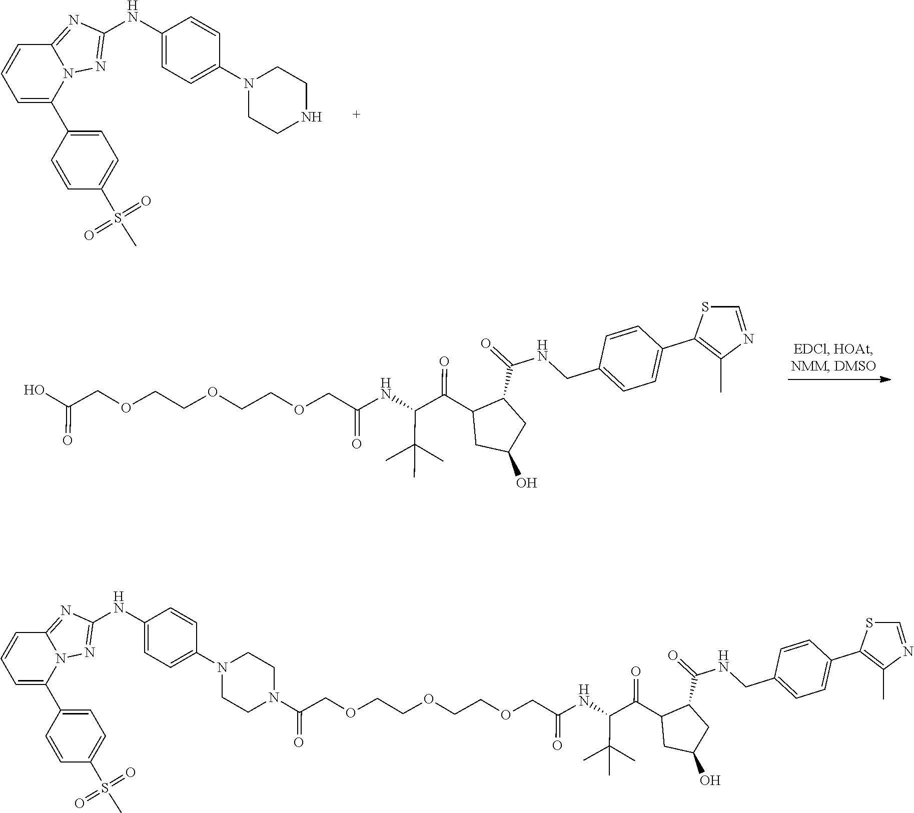 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CC2C(=O)[C@@H](NC(=O)COCCOCCOCC(=O)O)C(C)(C)C)cc1 ; Cc1ncsc1-c1ccc(CNC(=O)[C@@H]2C[C@@H](O)CC2C(=O)[C@@H](NC(=O)COCCOCCOCC(=O)N2CCN(c3ccc(Nc4nc5cccc(-c6ccc(S(C)(=O)=O)cc6)n5n4)cc3)CC2)C(C)(C)C)cc1]

JA-068 was synthesized following the standard procedure for preparing JA-010 (7.3 mg, 65% yield). MS (ESI) m/z: 1093.5 [M+H]⁺.

### Example 157. (2S,4R)-1-((S)-3,3-Dimethyl-2-(4-(4-(4-((5-(4-(methylsulfonyl)phenyl)-[1,2,4]triazolo[1,5-a]pyridin-2-yl)amino)phenyl)piperazin-1-yl)-4-oxobutanamido)butanoyl)-4-hydroxy-N-(4-(4-methylthiazol-5-yl)benzyl)pyrrolidine-2-carboxamide (JA-069)

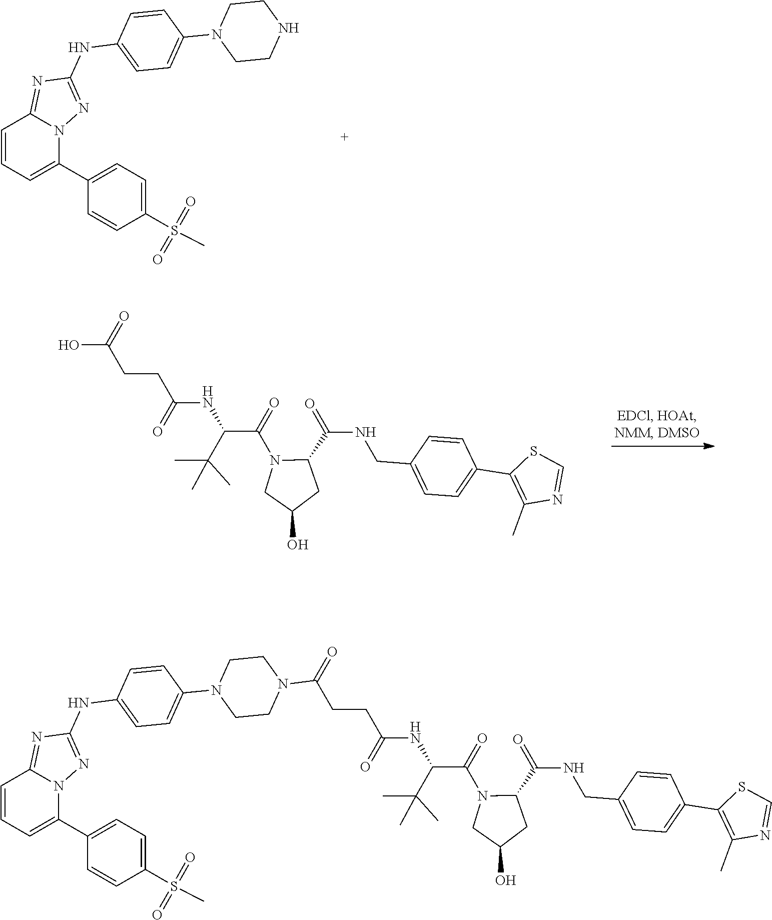 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CN2C(=O)[C@@H](NC(=O)CCC(=O)O)C(C)(C)C)cc1 ; Cc1ncsc1-c1ccc(CNC(=O)[C@@H]2C[C@@H](O)CN2C(=O)[C@@H](NC(=O)CCC(=O)N2CCN(c3ccc(Nc4nc5cccc(-c6ccc(S(C)(=O)=O)cc6)n5n4)cc3)CC2)C(C)(C)C)cc1]

JA-069 was synthesized following the standard procedure for preparing JA-010 (6.3 mg, 61% yield). MS (ESI) m/z: 961.4 [M+H]⁺.

### Example 158. (2S,4R)-1-((S)-3,3-Dimethyl-2-(3-(2-(3-(4-(4-((5-(4-(methylsulfonyl)phenyl)-[1,2,4]triazolo[1,5-a]pyridin-2-yl)amino)phenyl)piperazin-1-yl)-3-oxopropoxy)ethoxy)propanamido)butanoyl)-4-hydroxy-N-(4-(4-methylthiazol-5-yl)benzyl)pyrrolidine-2-carboxamide (JA-070)

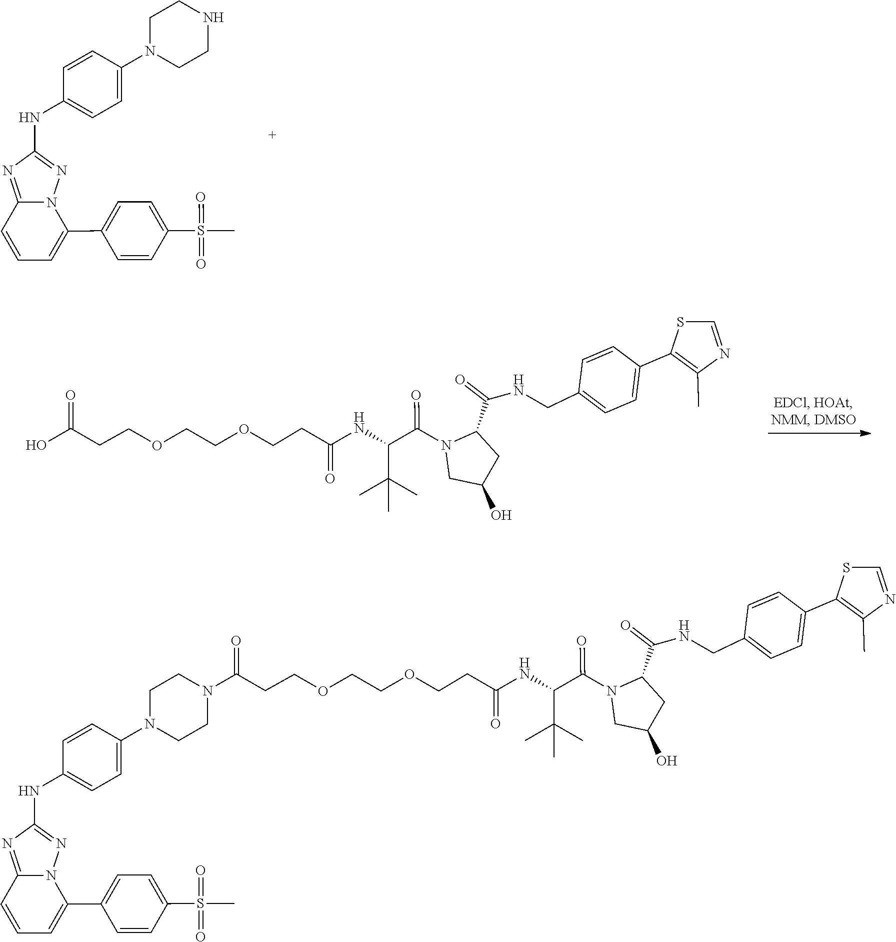 [SMILES: CS(=O)(=O)c1ccc(-c2cccc3nc(Nc4ccc(N5CCNCC5)cc4)nn23)cc1 ; Cc1ncsc1-c1ccc(CNC(=O)[C@@H]2C[C@@H](O)CN2C(=O)[C@@H](NC(=O)CCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCC(=O)N2CCN(c3ccc(Nc4nc5cccc(-c6ccc(S(C)(=O)=O)cc6)n5n4)cc3)CC2)C(C)(C)C)cc1]

JA-070 was synthesized following the standard procedure for preparing JA-010 (6.3 mg, 61% yield). MS (ESI) m/z: 1049.4 [M+H]⁺.

### Example 159. (2S,4R)-1-((S)-2-(tert-Butyl)-22-(4-(4-((5-(4-(methylsulfonyl)phenyl)-[1,2,4]triazolo[1,5-a]pyridin-2-yl)amino)phenyl)piperazin-1-yl)-4,22-dioxo-7,10,13,16,19-pentaoxa-3-azadocosanoyl)-4-hydroxy-N-(4-(4-methylthiazol-5-yl)benzyl)pyrrolidine-2-carboxamide (JA-071)

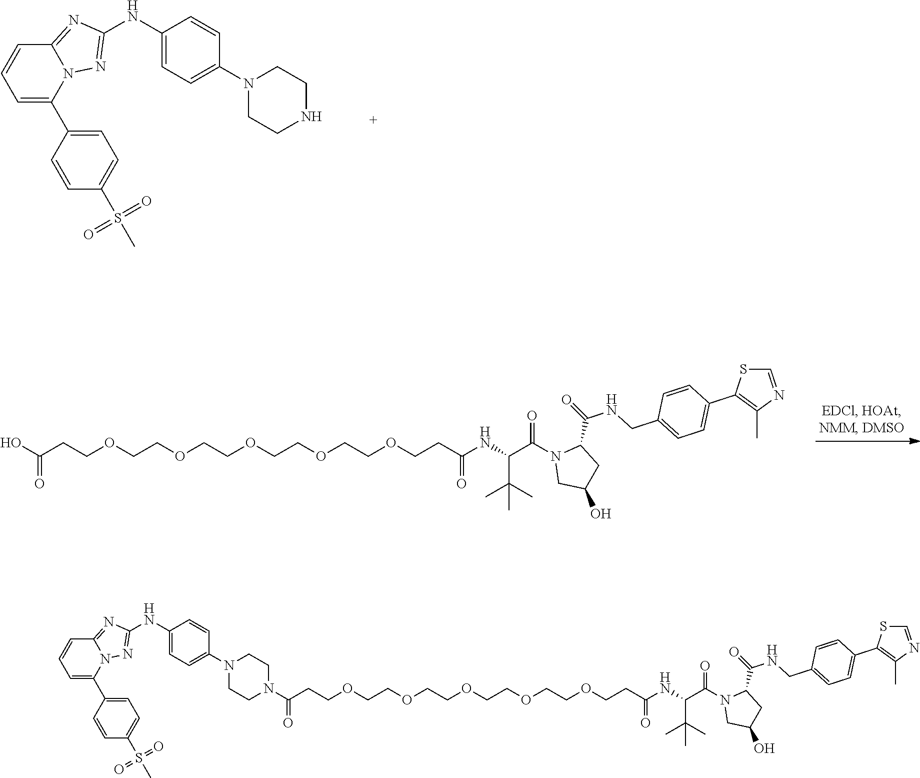 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)CCOCCOCCOCCOCCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCC(=O)N2CCN(c3ccc(Nc4nc5cccc(-c6ccc(S(C)(=O)=O)cc6)n5n4)cc3)CC2)C(C)(C)C)cc1]

JA-071 was synthesized following the standard procedure for preparing JA-010 (7.5 mg, 63% yield). MS (ESI) m/z: 1181.5 [M+H]⁺.

### Example 160. (2S,4R)-1-((S)-3,3-Dimethyl-2-(2-(2-(4-(4-((5-(4-(methylsulfonyl)phenyl)-[1,2,4]triazolo[1,5-a]pyridin-2-yl)amino)phenyl)piperazin-1-yl)-2-oxoethoxy)acetamido)butanoyl)-4-hydroxy-N-(4-(4-methylthiazol-5-yl)benzyl)pyrrolidine-2-carboxamide (JA-072)

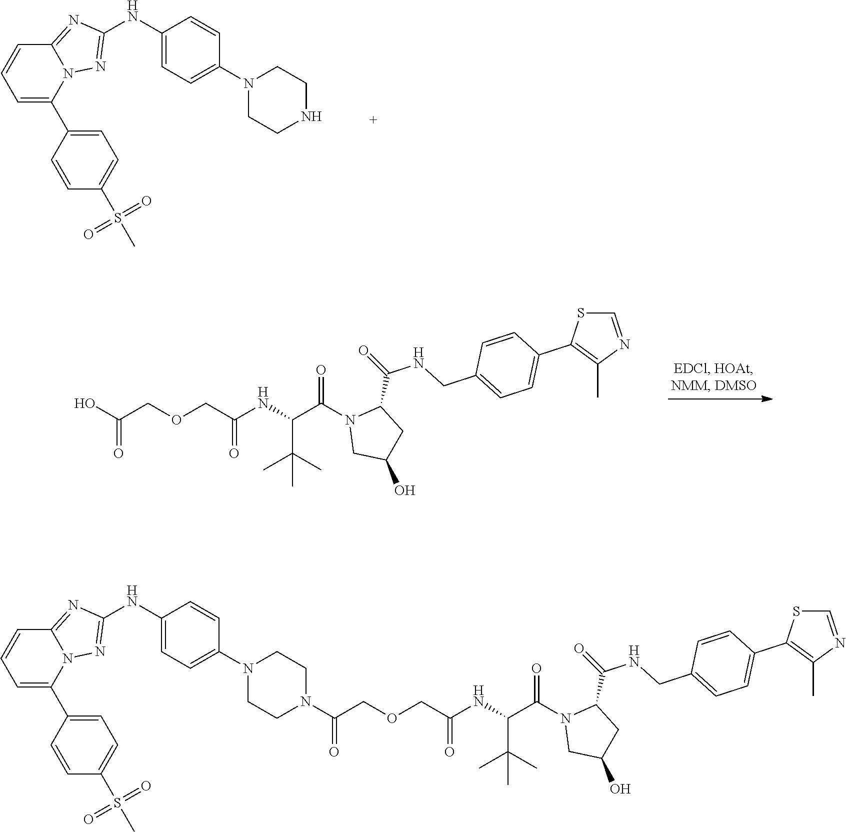 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)COCC(=O)O)C(C)(C)C)cc1 ; Cc1ncsc1-c1ccc(CNC(=O)[C@@H]2C[C@@H](O)CN2C(=O)[C@@H](NC(=O)COCC(=O)N2CCN(c3ccc(Nc4nc5cccc(-c6ccc(S(C)(=O)=O)cc6)n5n4)cc3)CC2)C(C)(C)C)cc1]

JA-072 was synthesized following the standard procedure for preparing JA-010 (6.2 mg, 60% yield). MS (ESI) m/z: 977.4 [M+H]⁺.

### Example 161. (2S,4R)-1-((S)-3,3-Dimethyl-2-(2-(2-(2-(4-(4-((5-(4-(methylsulfonyl)phenyl)-[1,2,4]triazolo[1,5-a]pyridin-2-yl)amino)phenyl)piperazin-1-yl)-2-oxoethoxy)ethoxy)acetamido)butanoyl)-4-hydroxy-N-(4-(4-methylthiazol-5-yl)benzyl)pyrrolidine-2-carboxamide (JA-073)

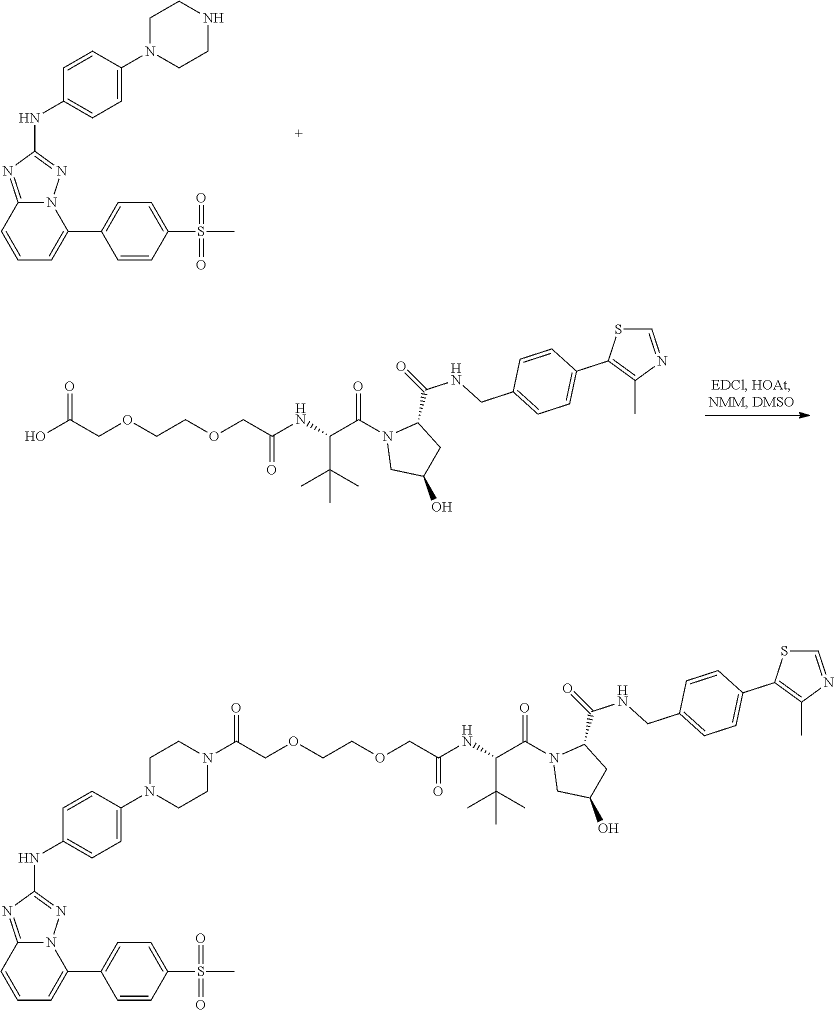 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)COCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCC(=O)N2CCN(c3ccc(Nc4nc5cccc(-c6ccc(S(C)(=O)=O)cc6)n5n4)cc3)CC2)C(C)(C)C)cc1]

JA-073 was synthesized following the standard procedure for preparing JA-010 (4.5 mg, 50% yield). MS (ESI) m/z: 1021.4 [M+H]⁺.

### Example 162. (2S,4R)-1-((S)-3,3-Dimethyl-2-(6-(4-(4-((5-(4-(methylsulfonyl)phenyl)-[1,2,4]triazolo[1,5-a]pyridin-2-yl)amino)phenyl)piperazin-1-yl)-6-oxohexanamido)butanoyl)-4-hydroxy-N-(4-(4-methylthiazol-5-yl)benzyl)pyrrolidine-2-carboxamide (JA-074)

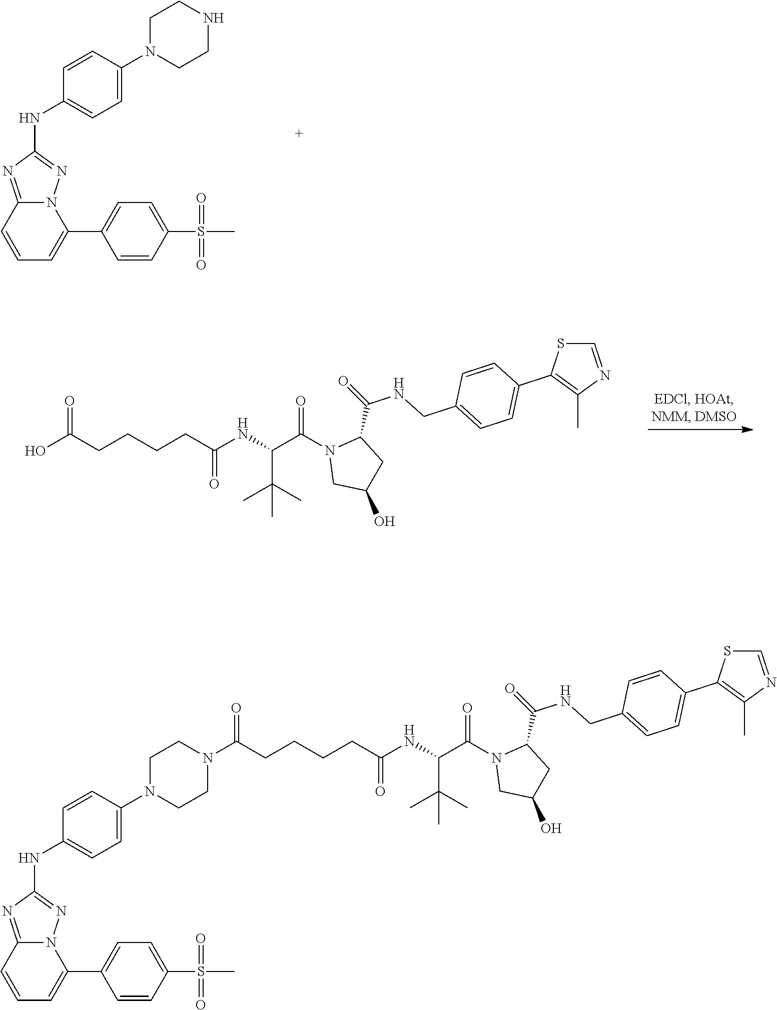 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)CCCCC(=O)O)C(C)(C)C)cc1 ; Cc1ncsc1-c1ccc(CNC(=O)[C@@H]2C[C@@H](O)CN2C(=O)[C@@H](NC(=O)CCCCC(=O)N2CCN(c3ccc(Nc4nc5cccc(-c6ccc(S(C)(=O)=O)cc6)n5n4)cc3)CC2)C(C)(C)C)cc1]

JA-074 was synthesized following the standard procedure for preparing JA-010 (5.5 mg, 55% yield). MS (ESI) m/z: 989.4 [M+H]⁺.

### Example 163. 2-(2,6-Dioxopiperidin-3-yl)-4-((2-(2-(3-(4-(4-((5-(4-(methylsulfonyl)phenyl)-[1,2,4]triazolo[1,5-a]pyridin-2-yl)amino)phenyl)piperazin-1-yl)-3-oxopropoxy)ethoxy)ethyl)amino)isoindoline-1,3-dione (JA-075)

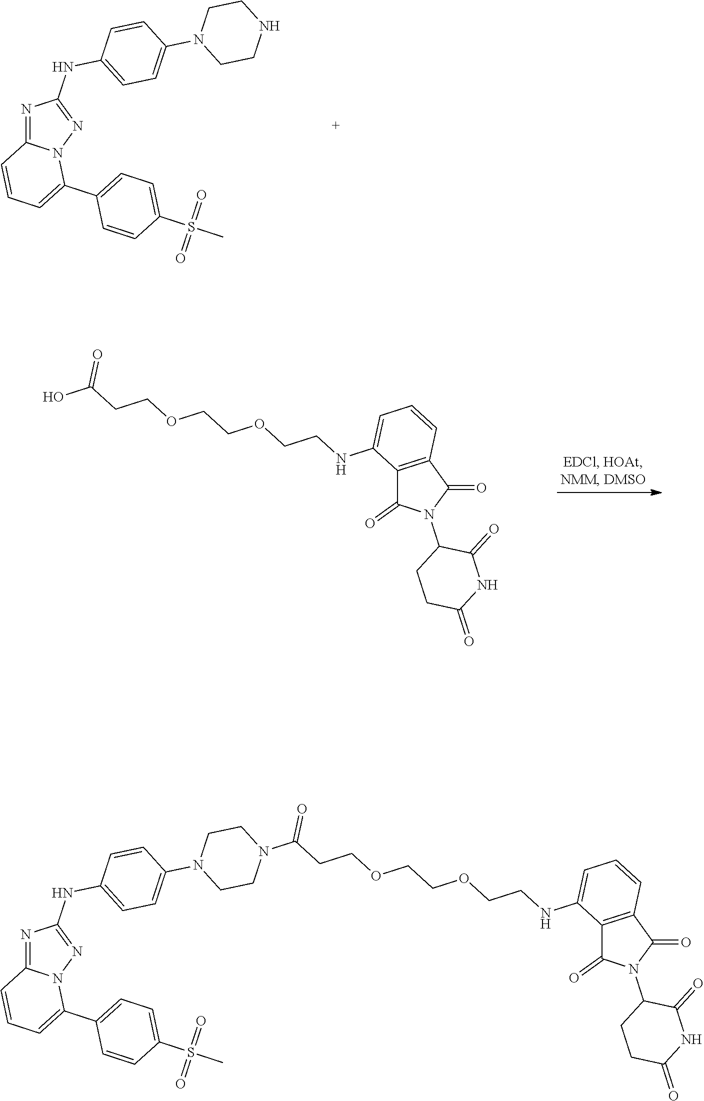 [SMILES: CS(=O)(=O)c1ccc(-c2cccc3nc(Nc4ccc(N5CCNCC5)cc4)nn23)cc1 ; "+" ; O=C(O)CCOCCOCCNc1cccc2c1C(=O)N(C1CCC(=O)NC1=O)C2=O ; CS(=O)(=O)c1ccc(-c2cccc3nc(Nc4ccc(N5CCN(C(=O)CCOCCOCCNc6cccc7c6C(=O)N(C6CCC(=O)NC6=O)C7=O)CC5)cc4)nn23)cc1]

JA-075 was synthesized following the standard procedure for preparing JA-010 (5.6 mg, 56% yield). MS (ESI) m/z: 864.3 [M+H]⁺.

### Example 164. (2S,4R)-1-((S)-3,3-Dimethyl-2-(7-(4-(4-((5-(4-(methylsulfonyl)phenyl)-[1,2,4]triazolo[1,5-a]pyridin-2-yl)amino)phenyl)piperazin-1-yl)-7-oxoheptanamido)butanoyl)-4-hydroxy-N-(4-(4-methylthiazol-5-yl)benzyl)pyrrolidine-2-carboxamide (JA-076)

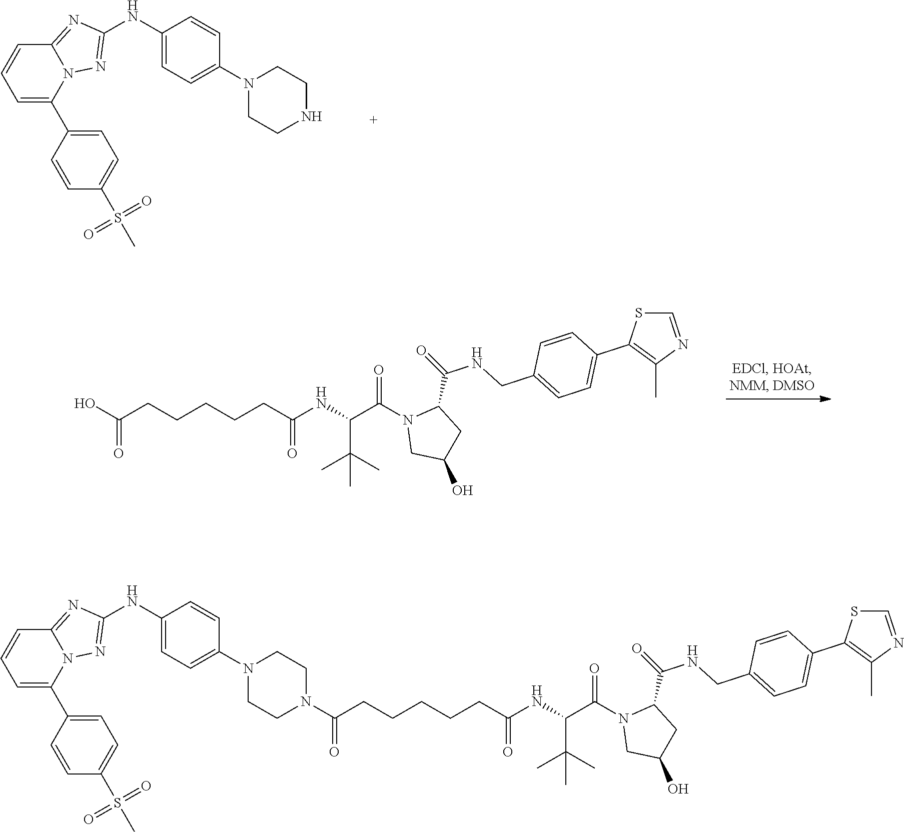 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)CCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCC(=O)N2CCN(c3ccc(Nc4nc5cccc(-c6ccc(S(C)(=O)=O)cc6)n5n4)cc3)CC2)C(C)(C)C)cc1]

JA-076 was synthesized following the standard procedure for preparing JA-010 (6.3 mg, 65% yield). MS (ESI) m/z: 1003.4 [M+H]⁺.

### Example 165. 2-(2,6-Dioxo-piperidin-3-yl)-4-(2-{2-[2-(2-{2-[3-(4-{4-[5-(4-methanesulfonyl-phenyl)-[1,2,4]triazolo[1,5-a]pyridin-2-ylamino]-phenyl}-piperazin-1-yl)-3-oxo-propoxy]-ethoxy}-ethoxy)-ethoxy]-ethoxy}-ethylamino)-isoindole-1,3-dione (JA-077)

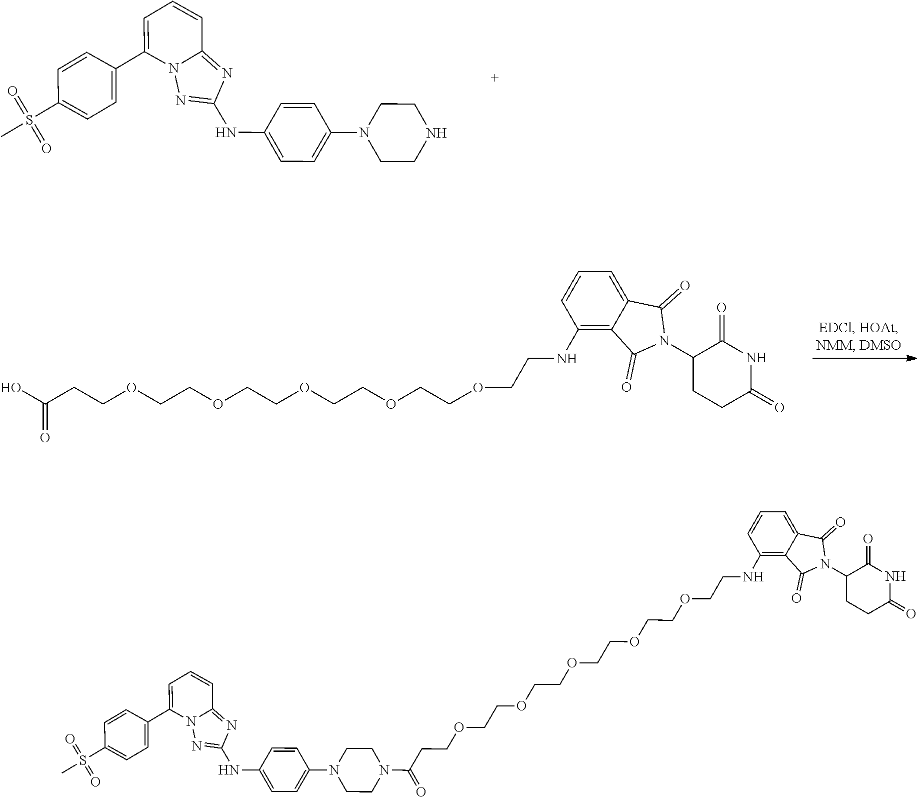 [SMILES: CS(=O)(=O)c1ccc(-c2cccc3nc(Nc4ccc(N5CCNCC5)cc4)nn23)cc1 ; "+" ; O=C(O)CCOCCOCCOCCOCCOCCNc1cccc2c1C(=O)N(C1CCC(=O)NC1=O)C2=O ; CS(=O)(=O)c1ccc(-c2cccc3nc(Nc4ccc(N5CCN(C(=O)CCOCCOCCOCCOCCOCCNc6cccc7c6C(=O)N(C6CCC(=O)NC6=O)C7=O)CC5)cc4)nn23)cc1]

JA-077 was synthesized following the standard procedure for preparing JA-010 (12 mg, 76% yield). MS (ESI) m/z: 996.4 [M+H]⁺.

### Example 166. 2-(2,6-Dioxo-piperidin-3-yl)-4-[7-(4-{4-[5-(4-methanesulfonyl-phenyl)-[1,2,4]triazolo[1,5-a]pyridin-2-ylamino]-phenyl}-piperazin-1-yl)-7-oxo-heptylamino]-isoindole-1,3-dione (JA-078)

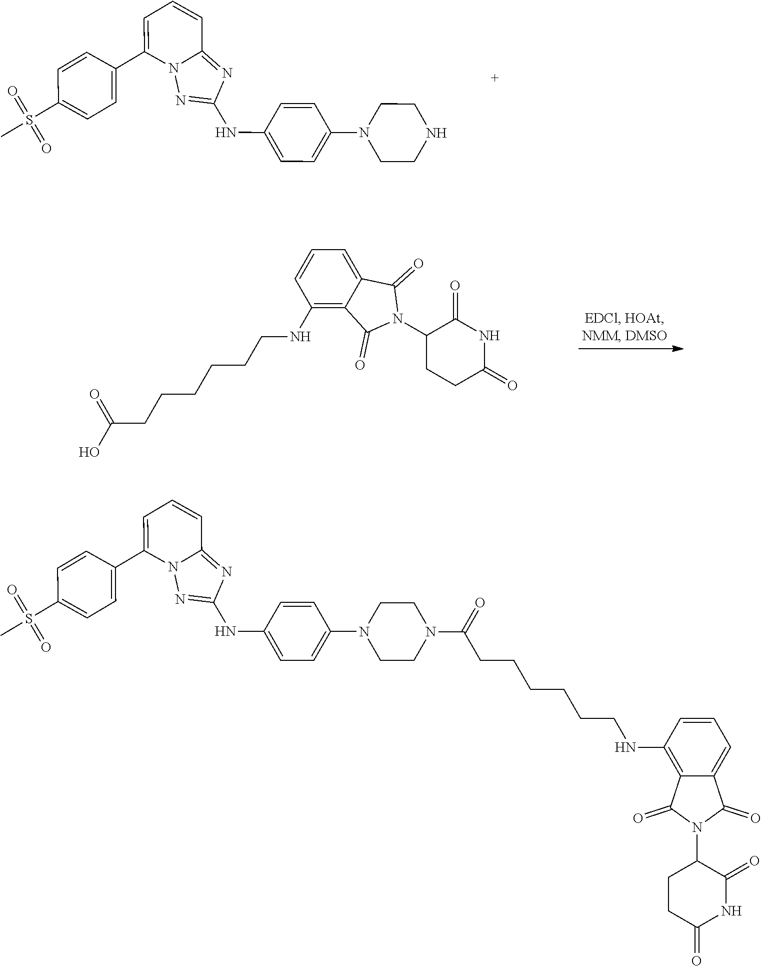 [SMILES: CS(=O)(=O)c1ccc(-c2cccc3nc(Nc4ccc(N5CCNCC5)cc4)nn23)cc1 ; "+" ; O=C(O)CCCCCCNc1cccc2c1C(=O)N(C1CCC(=O)NC1=O)C2=O ; CS(=O)(=O)c1ccc(-c2cccc3nc(Nc4ccc(N5CCN(C(=O)CCCCCCNc6cccc7c6C(=O)N(C6CCC(=O)NC6=O)C7=O)CC5)cc4)nn23)cc1]

JA-078 was synthesized following the standard procedure for preparing JA-010 (11 mg, 70% yield). MS (ESI) m/z: 832.3 [M+H]⁺.

### Example 167. 2-(2,6-Dioxo-piperidin-3-yl)-4-{2-[3-(4-{4-[5-(4-methanesulfonyl-phenyl)-[1,2,4]triazolo[1,5-a]pyridin-2-ylamino]-phenyl}-piperazin-1-yl)-3-oxo-propoxy]-ethylamino}-isoindole-1,3-dione (JA-079)

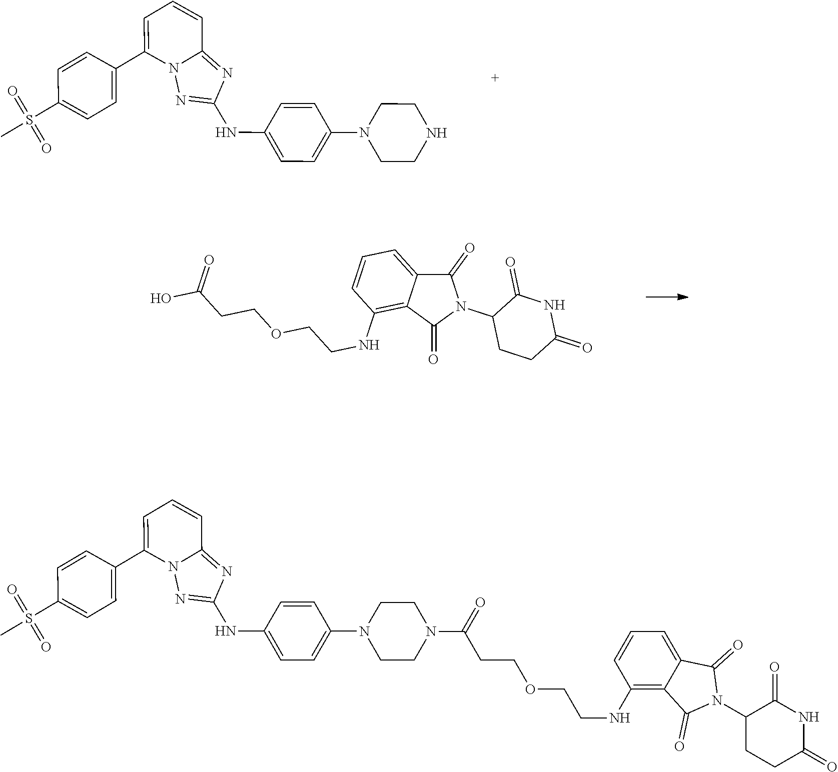 [SMILES: CS(=O)(=O)c1ccc(-c2cccc3nc(Nc4ccc(N5CCNCC5)cc4)nn23)cc1 ; "+" ; O=C(O)CCOCCNc1cccc2c1C(=O)N(C1CCC(=O)NC1=O)C2=O ; CS(=O)(=O)c1ccc(-c2cccc3nc(Nc4ccc(N5CCN(C(=O)CCOCCNc6cccc7c6C(=O)N(C6CCC(=O)NC6=O)C7=O)CC5)cc4)nn23)cc1]

JA-079 was synthesized following the standard procedure for preparing JA-010 (11 mg, 70% yield). MS (ESI) m/z: 820.3 [M+H]⁺.

### Example 168. 2-(2,6-Dioxo-piperidin-3-yl)-4-{2-[2-(2-{2-[3-(4-{4-[5-(4-methanesulfonyl-phenyl)-[1,2,4]triazolo[1,5-a]pyridin-2-ylamino]-phenyl}-piperazin-1-yl)-3-oxo-propoxy]-ethoxy}-ethoxy)-ethoxy]-ethylamino}-isoindole-1,3-dione (JA-080)

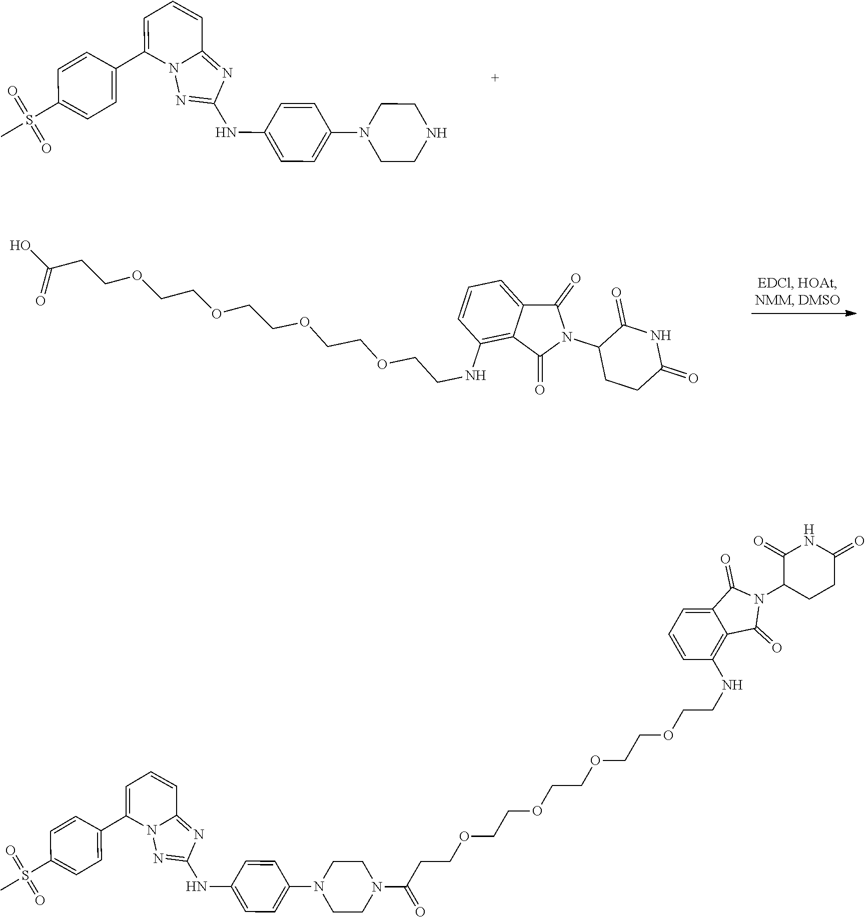 [SMILES: CS(=O)(=O)c1ccc(-c2cccc3nc(Nc4ccc(N5CCNCC5)cc4)nn23)cc1 ; "+" ; O=C(O)CCOCCOCCOCCOCCNc1cccc2c1C(=O)N(C1CCC(=O)NC1=O)C2=O ; CS(=O)(=O)c1ccc(-c2cccc3nc(Nc4ccc(N5CCN(C(=O)CCOCCOCCOCCOCCNc6cccc7c6C(=O)N(C6CCC(=O)NC6=O)C7=O)CC5)cc4)nn23)cc1]

JA-080 was synthesized following the standard procedure for preparing JA-010 (12 mg, 71% yield). MS (ESI) m/z: 952.3 [M+H]⁺.

### Example 169. 4-Hydroxy-1-{2-[11-(4-{4-[5-(4-methanesulfonyl-phenyl)-[1,2,4]triazolo[1,5-a]pyridin-2-ylamino]-phenyl}-piperazin-1-yl)-11-oxo-undecanoylamino]-3,3-dimethyl-butyryl}-pyrrolidine-2-carboxylic acid 4-(4-methyl-thiazol-5-yl)-benzylamide (JA-081)

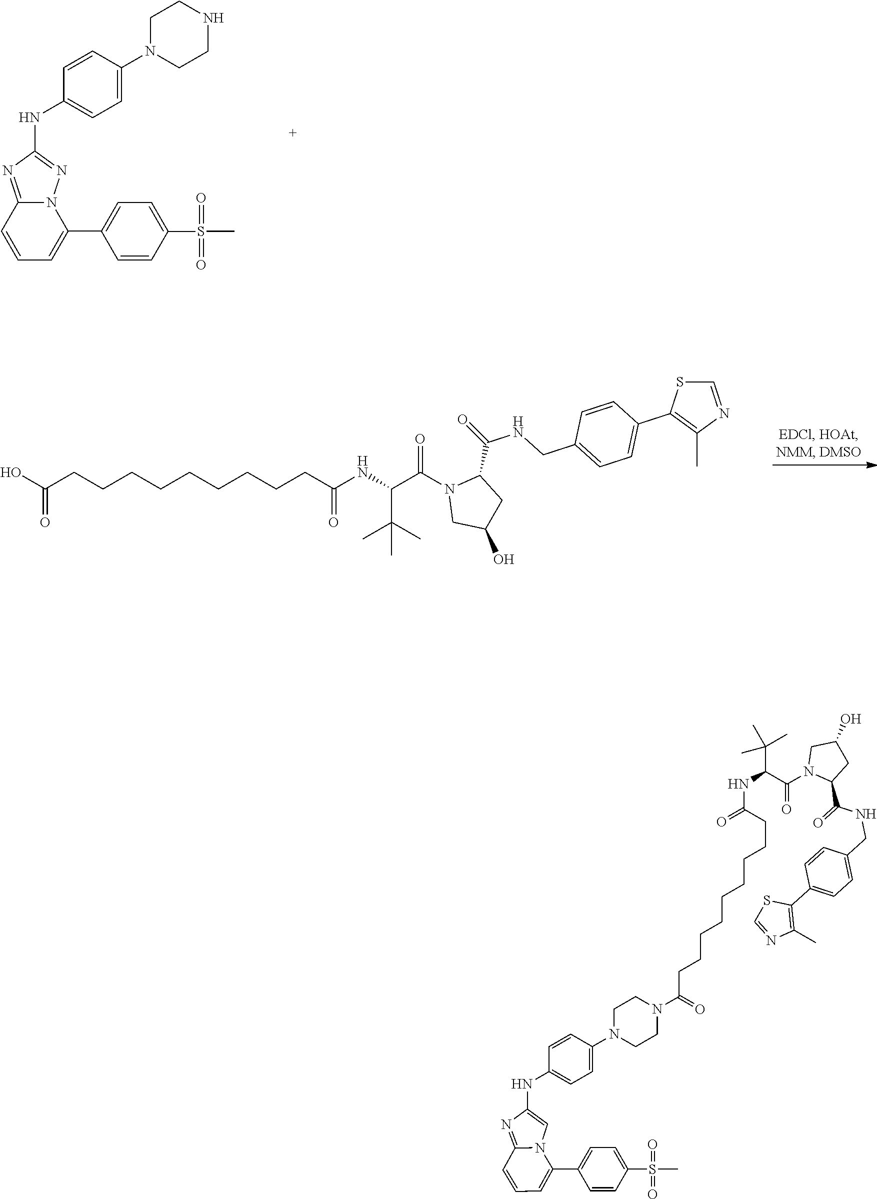 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)CCCCCCCCCC(=O)O)C(C)(C)C)cc1 ; Cc1ncsc1-c1ccc(CNC(=O)[C@@H]2C[C@@H](O)CN2C(=O)[C@@H](NC(=O)CCCCCCCCCC(=O)N2CCN(c3ccc(Nc4cn5c(-c6ccc(S(C)(=O)=O)cc6)cccc5n4)cc3)CC2)C(C)(C)C)cc1]

JA-081 was synthesized following the standard procedure for preparing JA-010 (5.2 mg, 51% yield). MS (ESI) m/z: 1059.5 [M+H]⁺.

### Example 170. 4-Hydroxy-1-(2-{3-[3-(4-{4-[5-(4-methanesulfonyl-phenyl)-[1,2,4]triazolo[1,5-a]pyridin-2-ylamino]-phenyl}-piperazin-1-yl)-3-oxo-propoxy]-propionylamino}-3,3-dimethyl-butyryl)-pyrrolidine-2-carboxylic acid 4-(4-methyl-thiazol-5-yl)-benzylamide (JA-082)

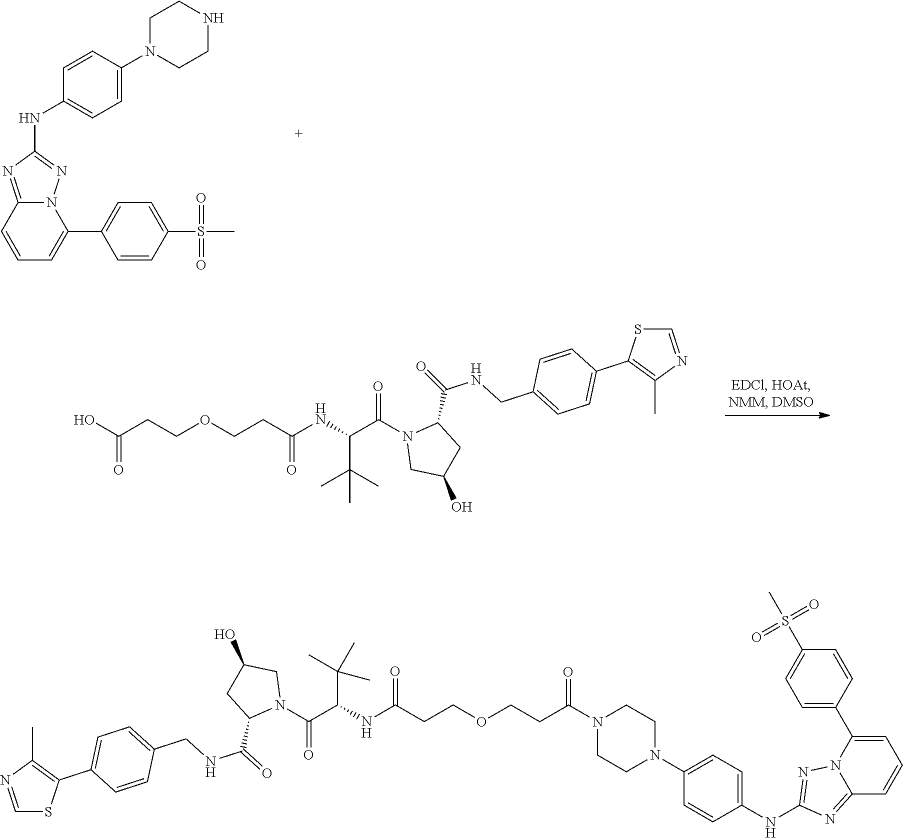 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)CCOCCC(=O)O)C(C)(C)C)cc1 ; Cc1ncsc1-c1ccc(CNC(=O)[C@@H]2C[C@@H](O)CN2C(=O)[C@@H](NC(=O)CCOCCC(=O)N2CCN(c3ccc(Nc4nc5cccc(-c6ccc(S(C)(=O)=O)cc6)n5n4)cc3)CC2)C(C)(C)C)cc1]

JA-082 was synthesized following the standard procedure for preparing JA-010 (5.5 mg, 50% yield). MS (ESI) m/z: 1005.4 [M+H]⁺.

### Example 171. (2S,4R)-1-((S)-2-(tert-Butyl)-20-(4-(4-((5-(4-(methylsulfonyl)phenyl)-[1,2,4]triazolo[1,5-a]pyridin-2-yl)amino)phenyl)piperazin-1-yl)-4,20-dioxo-6,9,12,15,18-pentaoxa-3-azaicosanoyl)-4-hydroxy-N-(4-(4-methylthiazol-5-yl)benzyl)pyrrolidine-2-carboxamide (JA-083)

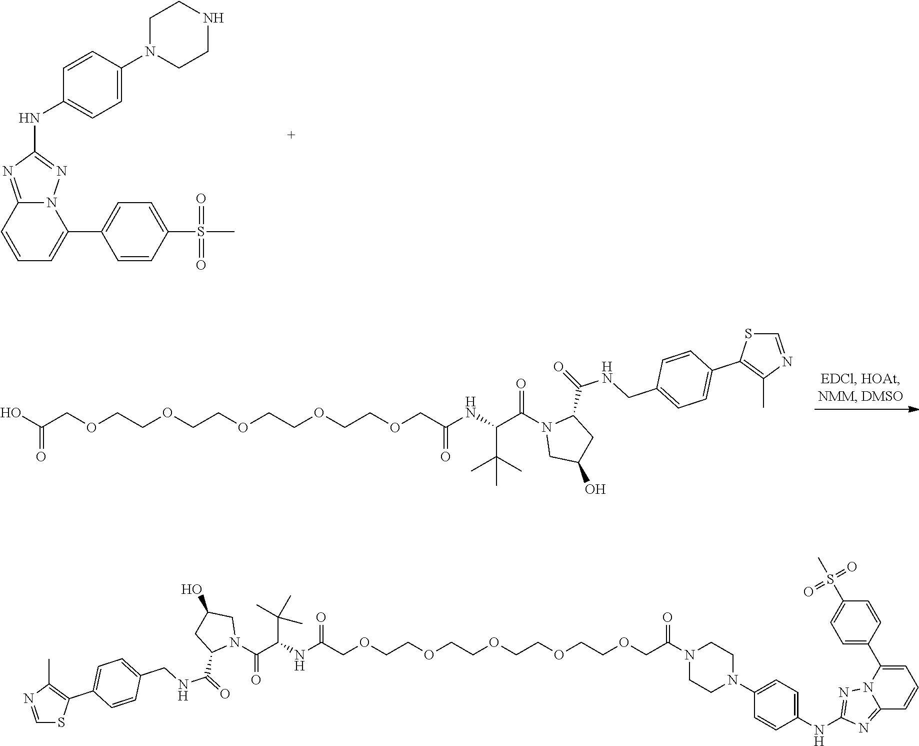 [SMILES: CS(=O)(=O)c1ccc(-c2cccc3nc(Nc4ccc(N5CCNCC5)cc4)nn23)cc1 ; "+" ; Cc1ncsc1-c1ccc(CNC(=O)[C@@H]2C[C@@H](O)CN2C(=O)[C@@H](NC(=O)COCCOCCOCCOCCOCC(=O)O)C(C)(C)C)cc1 ; Cc1ncsc1-c1ccc(CNC(=O)[C@@H]2C[C@@H](O)CN2C(=O)[C@@H](NC(=O)COCCOCCOCCOCCOCC(=O)N2CCN(c3ccc(Nc4nc5cccc(-c6ccc(S(C)(=O)=O)cc6)n5n4)cc3)CC2)C(C)(C)C)cc1]

JA-083 was synthesized following the standard procedure for preparing JA-010 (5.7 mg, 51% yield). MS (ESI) m/z: 1153.5 [M+H]⁺.

### Example 172. 2-(2,6-Dioxo-piperidin-3-yl)-4-[8-(4-{4-[5-(4-methanesulfonyl-phenyl)-[1,2,4]triazolo[1,5-a]pyridin-2-ylamino]-phenyl}-piperazin-1-yl)-8-oxo-octylamino]-isoindole-1,3-dione (JA-084)

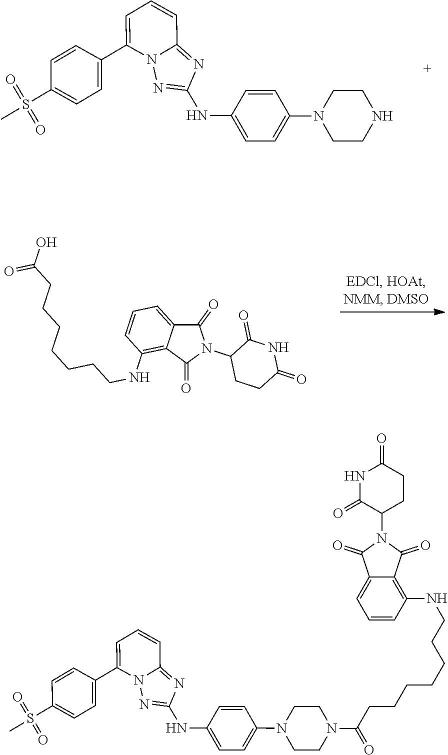 [SMILES: CS(=O)(=O)c1ccc(-c2cccc3nc(Nc4ccc(N5CCNCC5)cc4)nn23)cc1 ; "+" ; O=C(O)CCCCCCCNc1cccc2c1C(=O)N(C1CCC(=O)NC1=O)C2=O ; CS(=O)(=O)c1ccc(-c2cccc3nc(Nc4ccc(N5CCN(C(=O)CCCCCCCNc6cccc7c6C(=O)N(C6CCC(=O)NC6=O)C7=O)CC5)cc4)nn23)cc1]

JA-084 was synthesized following the standard procedure for preparing JA-010 (10 mg, 71% yield). MS (ESI) m/z: 846.3 [M+H]⁺.

### Example 173. 2-(2,6-Dioxo-piperidin-3-yl)-4-[2-(2-{2-[3-(4-{4-[5-(4-methanesulfonyl-phenyl)-[1,2,4]triazolo[1,5-a]pyridin-2-ylamino]-phenyl}-piperazin-1-yl)-3-oxo-propoxy]-ethoxy}-ethoxy)-ethylamino]-isoindole-1,3-dione (JA-085)

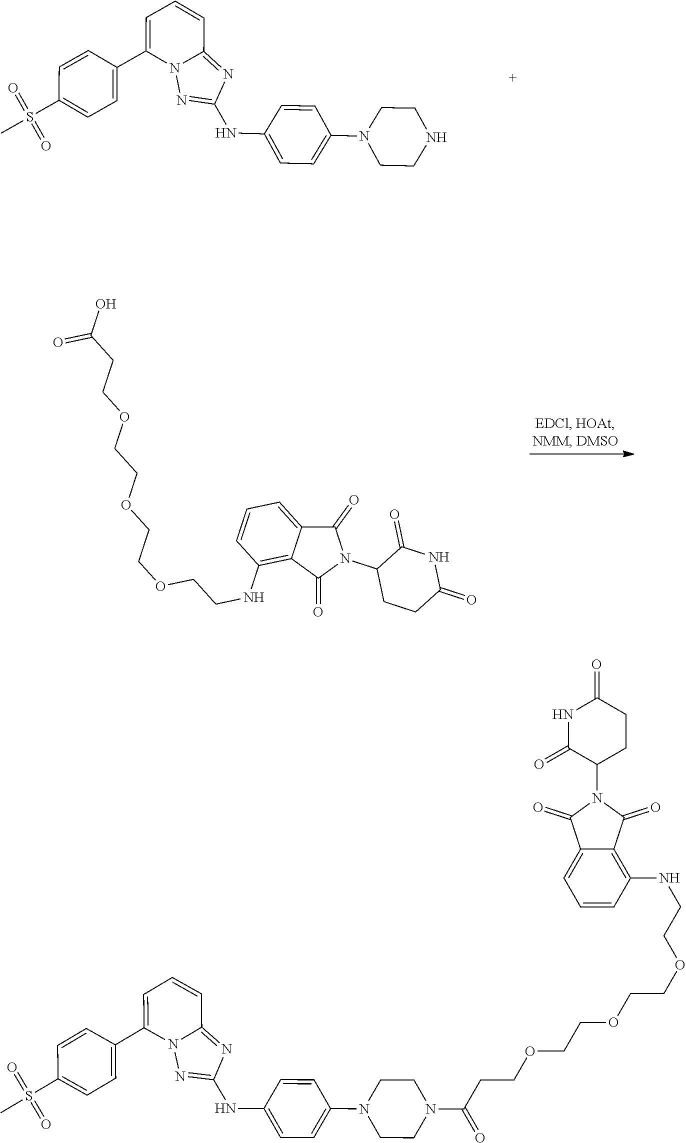 [SMILES: CS(=O)(=O)c1ccc(-c2cccc3nc(Nc4ccc(N5CCNCC5)cc4)nn23)cc1 ; "+" ; O=C(O)CCOCCOCCOCCNc1cccc2c1C(=O)N(C1CCC(=O)NC1=O)C2=O ; CS(=O)(=O)c1ccc(-c2cccc3nc(Nc4ccc(N5CCN(C(=O)CCOCCOCCOCCNc6cccc7c6C(=O)N(C6CCC(=O)NC6=O)C7=O)CC5)cc4)nn23)cc1]

JA-085 was synthesized following the standard procedure for preparing JA-010 (10 mg, 72% yield). MS (ESI) m/z: 908.3 [M+H]⁺.

### Example 174. 2-(4-(4-((4-((3-(N-(tert-Butyl)sulfamoyl)phenyl)amino)-5-methylpyrimidin-2-yl)amino)phenyl)piperazin-1-yl)-N-(2-(2-(2-((2-(2,6-dioxopiperidin-3-yl)-1,3-dioxoisoindolin-4-yl)amino)ethoxy)ethoxy)ethyl)acetamide (JA-086)

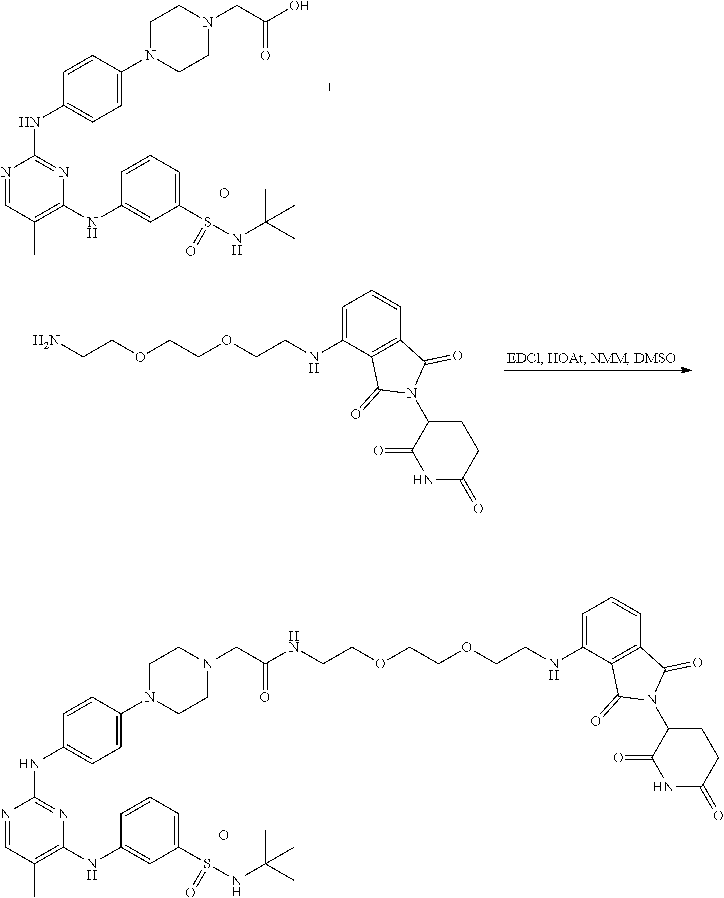

JA-086 was synthesized following the standard procedure for preparing JA-015 (17.6 mg, 99% yield). MS (ESI) m/z: 940.8 [M+H]⁺.

### Example 175. 2-(4-(4-((4-((3-(N-(tert-Butyl)sulfamoyl)phenyl)amino)-5-methylpyrimidin-2-yl)amino)phenyl)piperazin-1-yl)-N-(2-(2-((2-(2,6-dioxopiperidin-3-yl)-1,3-dioxoisoindolin-4-yl)amino)ethoxy)ethyl)acetamide (JA-087)

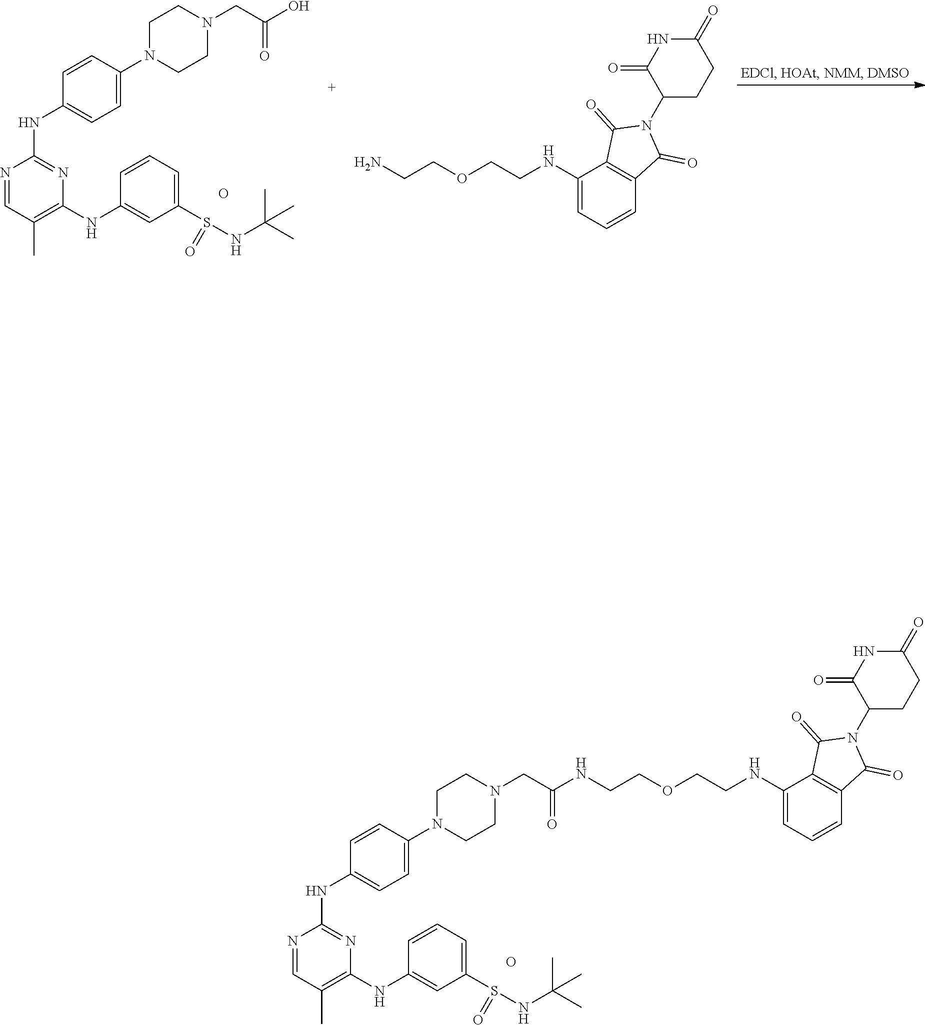

JA-087 was synthesized following the standard procedure for preparing JA-015 (9.5 mg, 59% yield). MS (ESI) m/z: 896.5 [M+H]⁺.

### Example 176. 2-(4-(4-((4-((3-(N-(tert-Butyl)sulfamoyl)phenyl)amino)-5-methylpyrimidin-2-yl)amino)phenyl)piperazin-1-yl)-N-(2-(2-(2-(2-((2-(2,6-dioxopiperidin-3-yl)-1,3-dioxoisoindolin-4-yl)amino)ethoxy)ethoxy)ethoxy)ethyl)acetamide (JA-088)

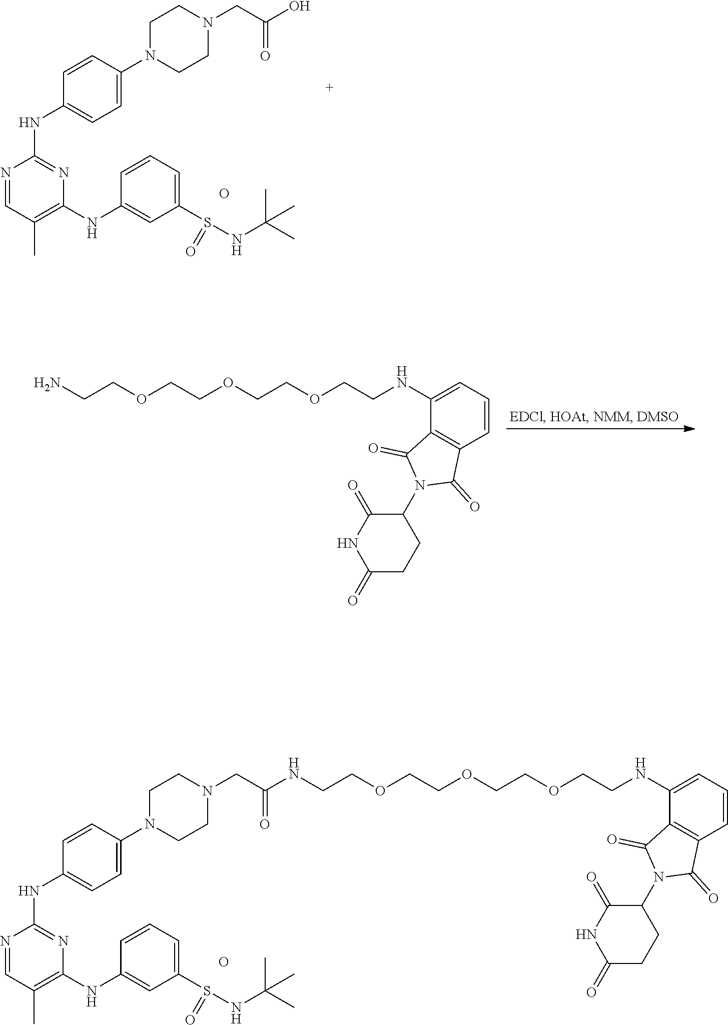

JA-088 was synthesized following the standard procedure for preparing JA-015 (11 mg, 62% yield). MS (ESI) m/z: 984.8 [M+H]⁺.

### Example 177. 2-(4-(4-((4-((3-(N-(tert-Butyl)sulfamoyl)phenyl)amino)-5-methylpyrimidin-2-yl)amino)phenyl)piperazin-1-yl)-N-(14-((2-(2,6-dioxopiperidin-3-yl)-1,3-dioxoisoindolin-4-yl)amino)-3,6,9,12-tetraoxatetradecyl)acetamide (JA-089)

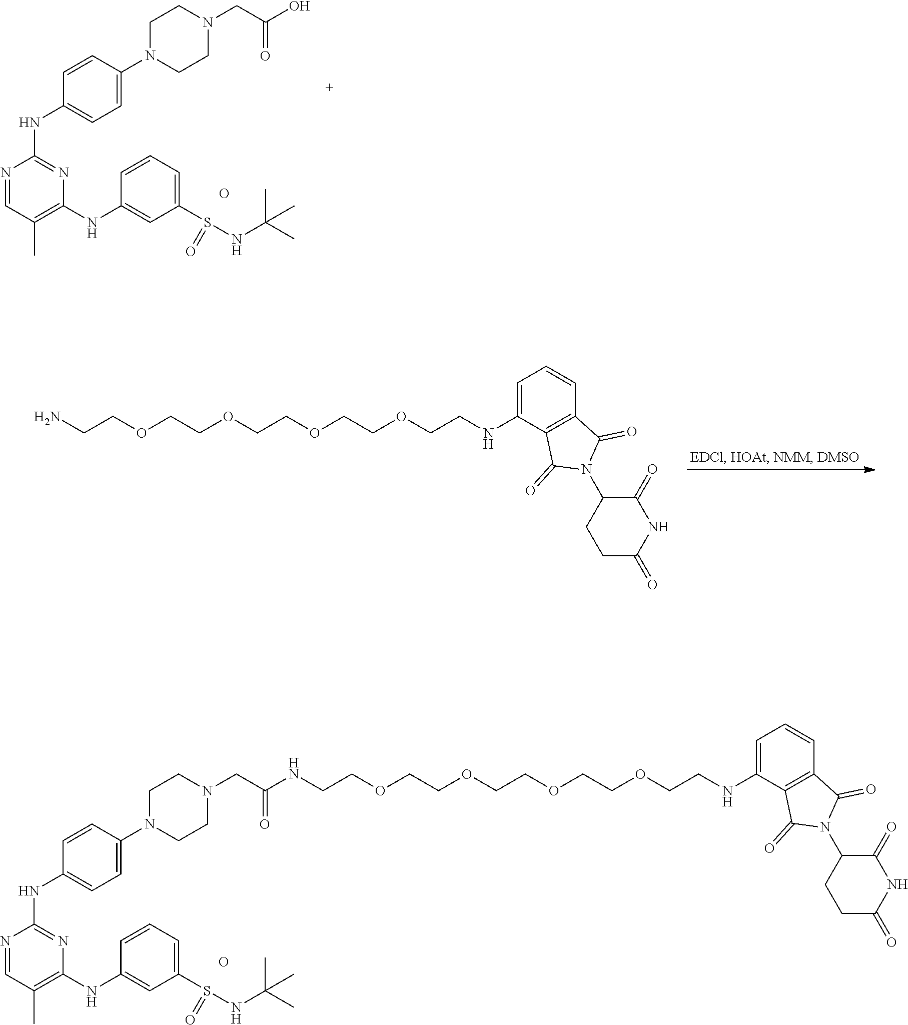

JA-089 was synthesized following the standard procedure for preparing JA-015 (5.6 mg, 30% yield). MS (ESI) m/z: 1050.8 [M+H]⁺.

### Example 178. 2-(4-(4-((4-((3-(N-(tert-Butyl)sulfamoyl)phenyl)amino)-5-methylpyrimidin-2-yl)amino)phenyl)piperazin-1-yl)-N-(2-((2-(2,6-dioxopiperidin-3-yl)-1,3-dioxoisoindolin-4-yl)amino)ethyl)acetamide (JA-090)

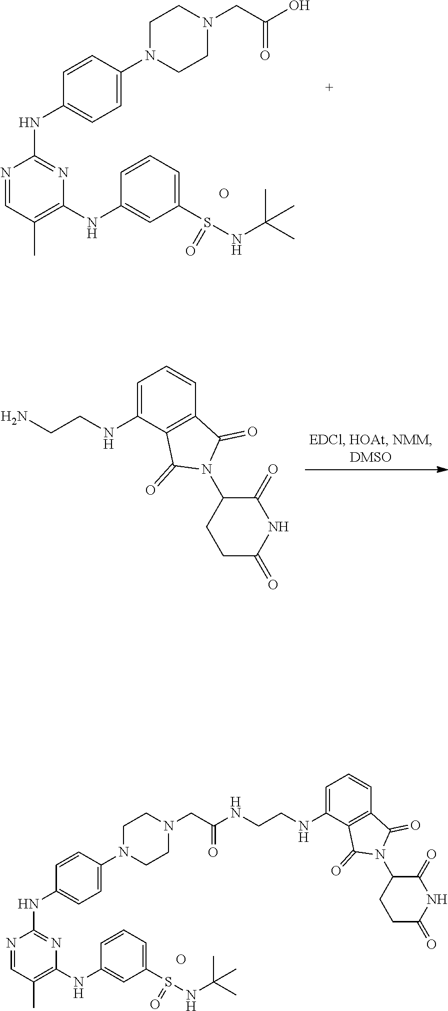

JA-090 was synthesized following the standard procedure for preparing JA-015 (4 mg, 26% yield). MS (ESI) m/z: 852.7 [M+H]⁺.

### Example 179. 2-(4-(4-((4-((3-(N-(tert-Butyl)sulfamoyl)phenyl)amino)-5-methylpyrimidin-2-yl)amino)phenyl)piperazin-1-yl)-N-(3-((2-(2,6-dioxopiperidin-3-yl)-1,3-dioxoisoindolin-4-yl)amino)propyl)acetamide (JA-091)

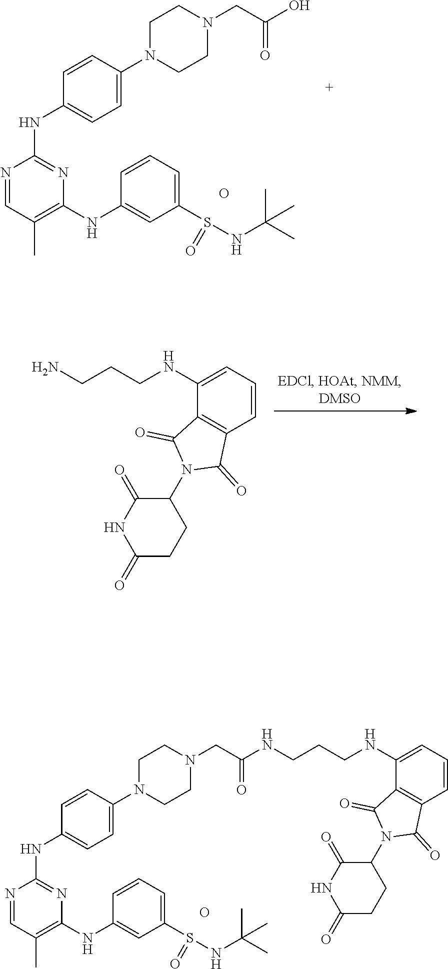

JA-091 was synthesized following the standard procedure for preparing JA-015 (11.7 mg, 75% yield). MS (ESI) m/z: 866.6 [M+H]⁺.

### Example 180. 2-(4-(4-((4-((3-(N-(tert-Butyl)sulfamoyl)phenyl)amino)-5-methylpyrimidin-2-yl)amino)phenyl)piperazin-1-yl)-N-(4-((2-(2,6-dioxopiperidin-3-yl)-1,3-dioxoisoindolin-4-yl)amino)butyl)acetamide (JA-092)

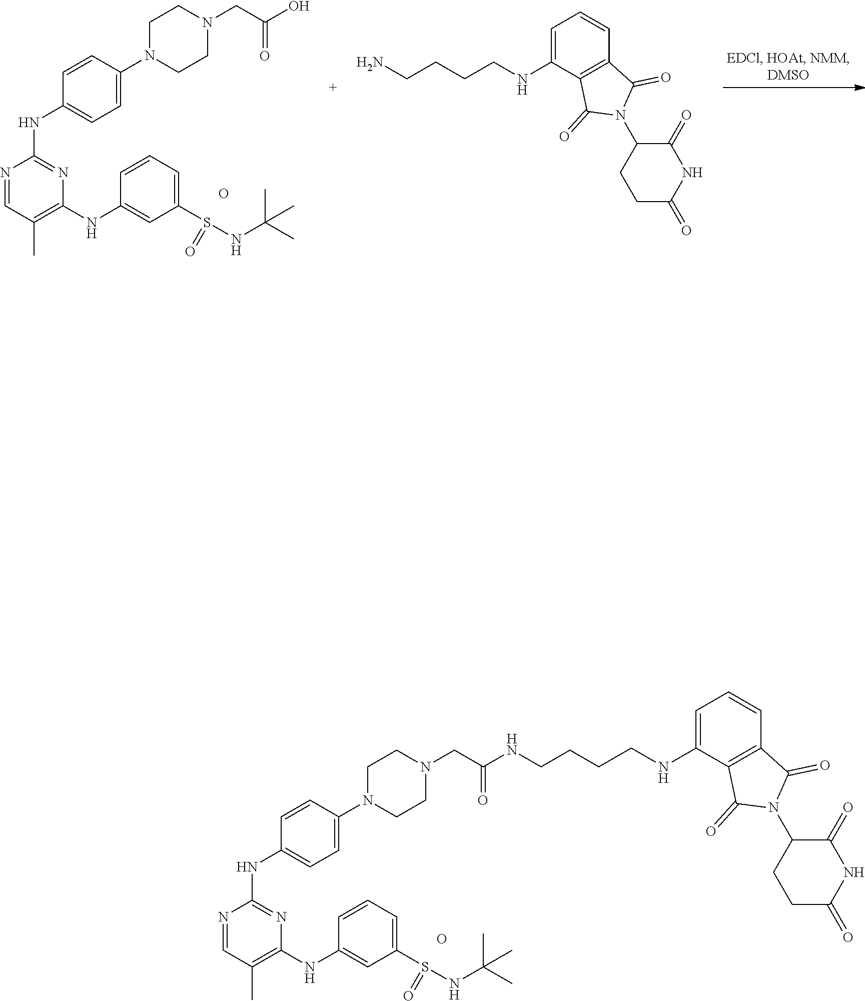

JA-092 was synthesized following the standard procedure for preparing JA-015 (12.4 mg, 78% yield). MS (ESI) m/z: 880.8 [M+H]⁺.

### Example 181. 2-(4-(4-((4-((3-(N-(tert-Butyl)sulfamoyl)phenyl)amino)-5-methylpyrimidin-2-yl)amino)phenyl)piperazin-1-yl)-N-(5-((2-(2,6-dioxopiperidin-3-yl)-1,3-dioxoisoindolin-4-yl)amino)pentyl)acetamide (JA-093)

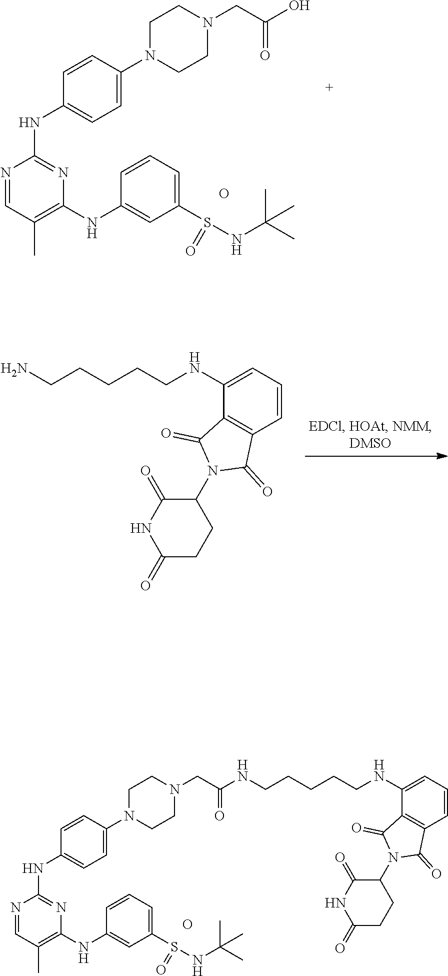

JA-093 was synthesized following the standard procedure for preparing JA-015 (5.9 mg, 37% yield). MS (ESI) m/z: 894.8 [M+H]⁺.

### Example 182. 2-(4-(4-((4-((3-(N-(tert-Butyl)sulfamoyl)phenyl)amino)-5-methylpyrimidin-2-yl)amino)phenyl)piperazin-1-yl)-N-(6-((2-(2,6-dioxopiperidin-3-yl)-1,3-dioxoisoindolin-4-yl)amino)hexyl)acetamide (JA-094)

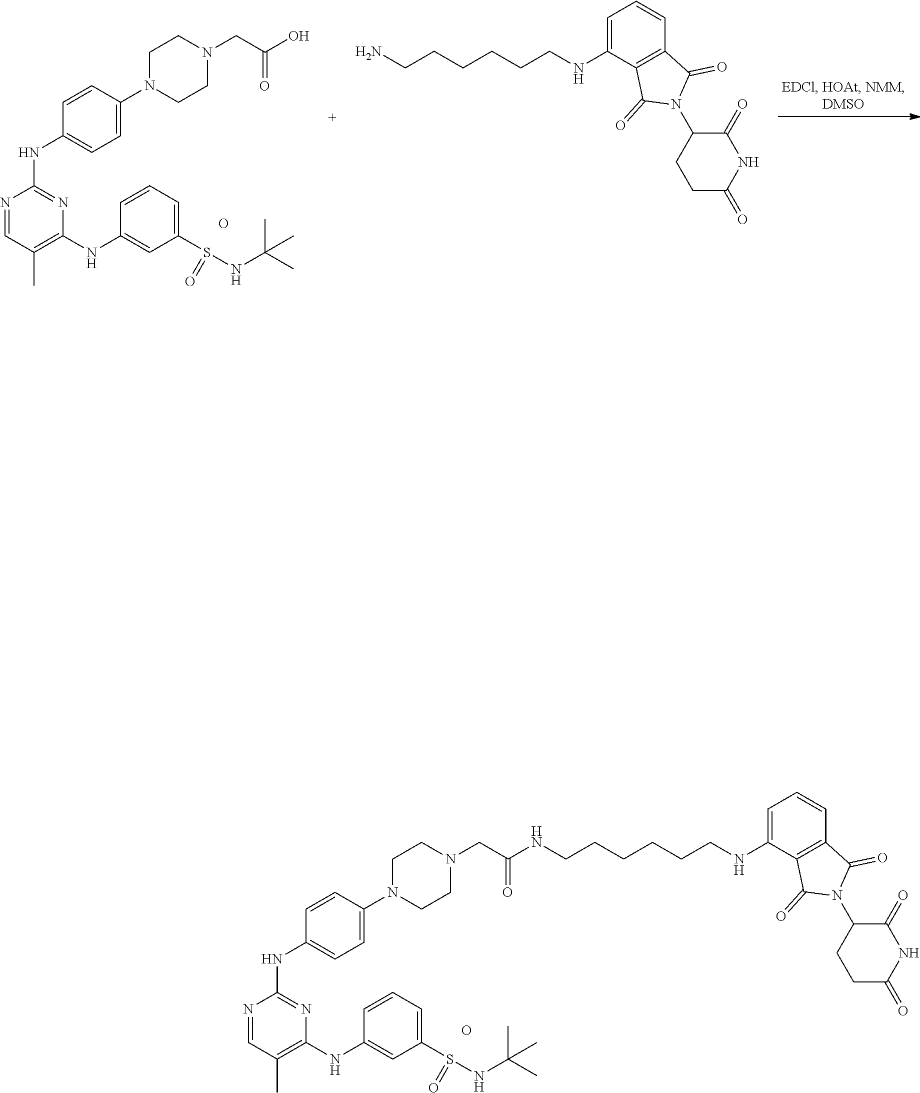

JA-094 was synthesized following the standard procedure for preparing JA-015 (7.0 mg, 43% yield). MS (ESI) m/z: 909.1 [M+H]⁺.

### Example 183. 2-(4-(4-((4-((3-(N-(tert-Butyl)sulfamoyl)phenyl)amino)-5-methylpyrimidin-2-yl)amino)phenyl)piperazin-1-yl)-N-(7-((2-(2,6-dioxopiperidin-3-yl)-1,3-dioxoisoindolin-4-yl)amino)heptyl)acetamide (JA-095)

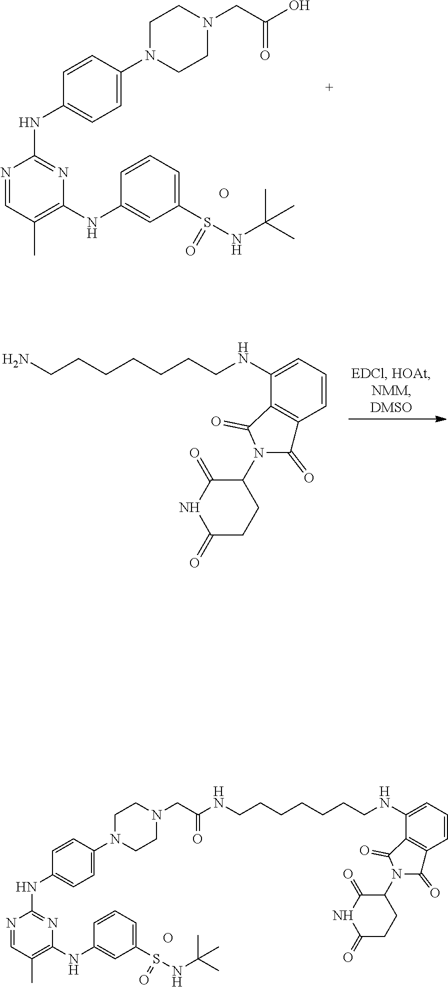

JA-095 was synthesized following the standard procedure for preparing JA-015 (12 mg, 72% yield). MS (ESI) m/z: 922.9 [M+H]⁺.

### Example 184. 2-(4-(4-((4-((3-(N-(tert-Butyl)sulfamoyl)phenyl)amino)-5-methylpyrimidin-2-yl)amino)phenyl)piperazin-1-yl)-N-(8-((2-(2,6-dioxopiperidin-3-yl)-1,3-dioxoisoindolin-4-yl)amino)octyl)acetamide (JA-096)

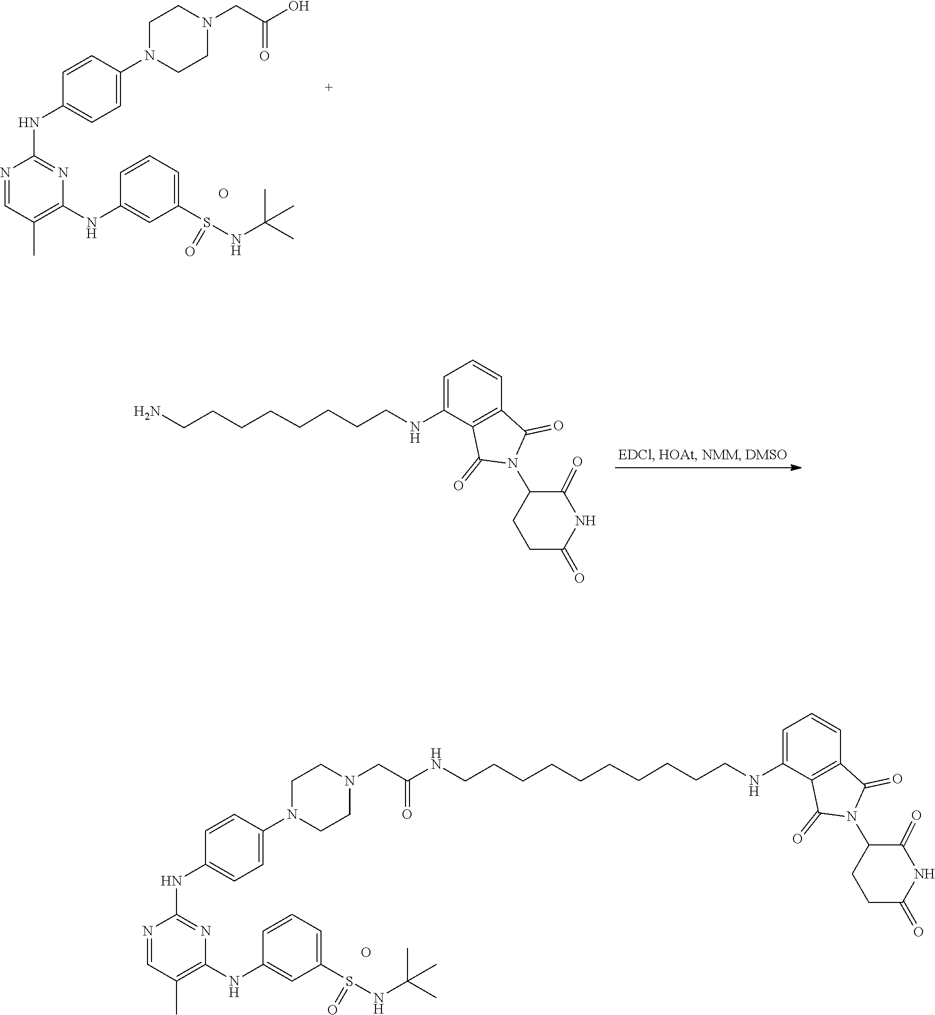

JA-096 was synthesized following the standard procedure for preparing JA-015 (9.5 mg, 56% yield). MS (ESI) m/z: 936.5 [M+H]⁺.

### Example 185. (2S,4R)-1-((S)-2-(2-(2-(4-(4-((4-((3-(N-(tert-Butyl)sulfamoyl)phenyl)amino)-5-methylpyrimidin-2-yl)amino)phenyl)piperazin-1-yl)acetamido)acetamido)-3,3-dimethylbutanoyl)-4-hydroxy-N-(4-(4-methylthiazol-5-yl)benzyl)pyrrolidine-2-carboxamide (JA-097)

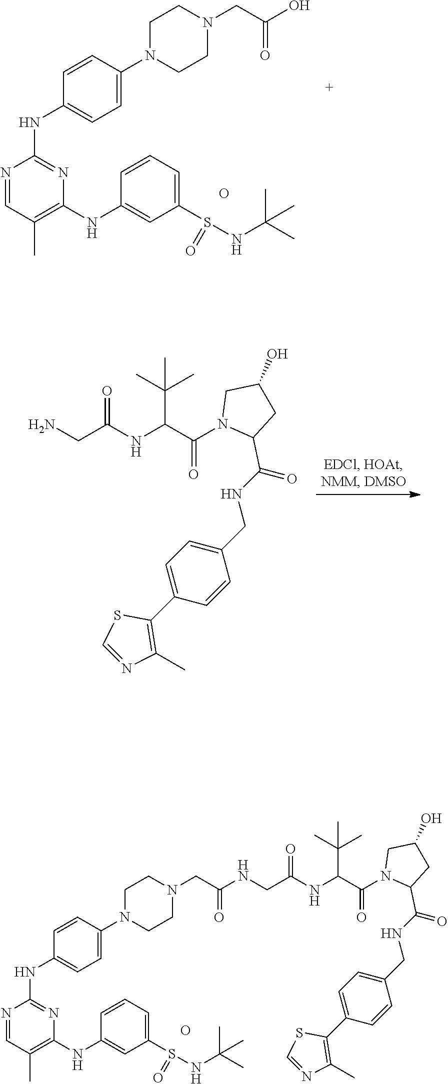

JA-097 was synthesized following the standard procedure for preparing JA-015 (9 mg, 49% yield). MS (ESI) m/z: 1024.3 [M+H]⁺.

### Example 186. (2S,4R)-1-((S)-2-(3-(2-(4-(4-((4-((3-(N-(tert-Butyl)sulfamoyl)phenyl)amino)-5-methylpyrimidin-2-yl)amino)phenyl)piperazin-1-yl)acetamido)propanamido)-3,3-dimethylbutanoyl)-4-hydroxy-N-(4-(4-methylthiazol-5-yl)benzyl)pyrrolidine-2-carboxamide (JA-098)

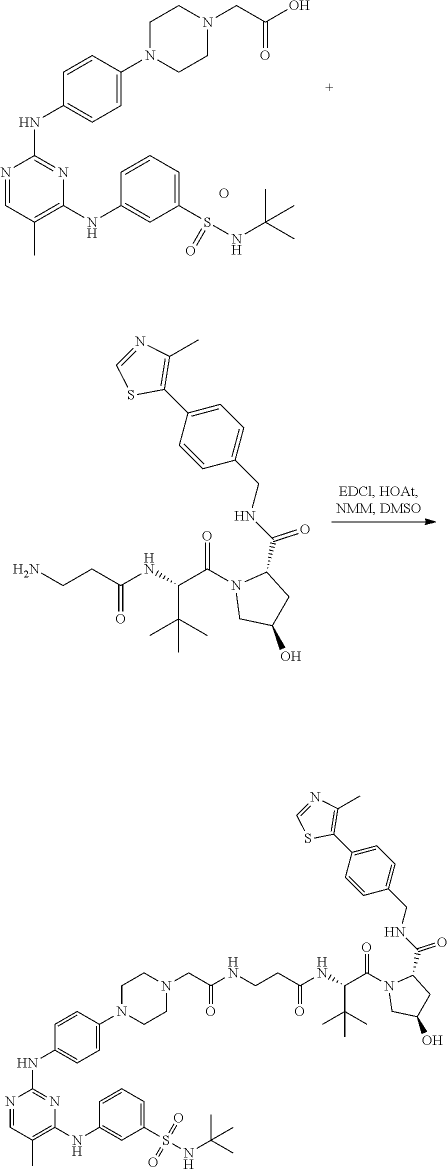

JA-098 was synthesized following the standard procedure for preparing JA-015 (2.7 mg, 14% yield). MS (ESI) m/z: 1038.7 [M+H]⁺.

### Example 187. (2S,4R)-1-((S)-2-(4-(2-(4-(4-((4-((3-(N-(tert-Butyl)sulfamoyl)phenyl)amino)-5-methylpyrimidin-2-yl)amino)phenyl)piperazin-1-yl)acetamido)butanamido)-3,3-dimethylbutanoyl)-4-hydroxy-N-(4-(4-methylthiazol-5-yl)benzyl)pyrrolidine-2-carboxamide (JA-099)

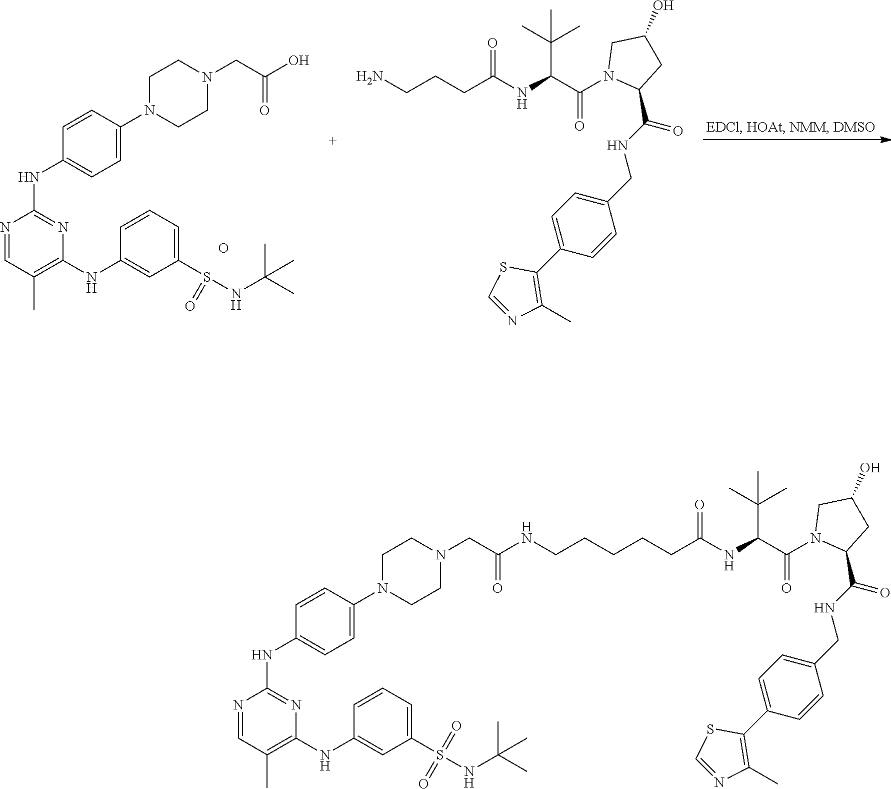

JA-099 was synthesized following the standard procedure for preparing JA-015 (3.2 mg, 17% yield). MS (ESI) m/z: 1051.5 [M+H]⁺.

### Example 188. (2S,4R)-1-((S)-2-(5-(2-(4-(4-((4-((3-(N-(tert-Butyl)sulfamoyl)phenyl)amino)-5-methylpyrimidin-2-yl)amino)phenyl)piperazin-1-yl)acetamido)pentanamido)-3,3-dimethylbutanoyl)-4-hydroxy-N-(4-(4-methylthiazol-5-yl)benzyl)pyrrolidine-2-carboxamide (JA-100)

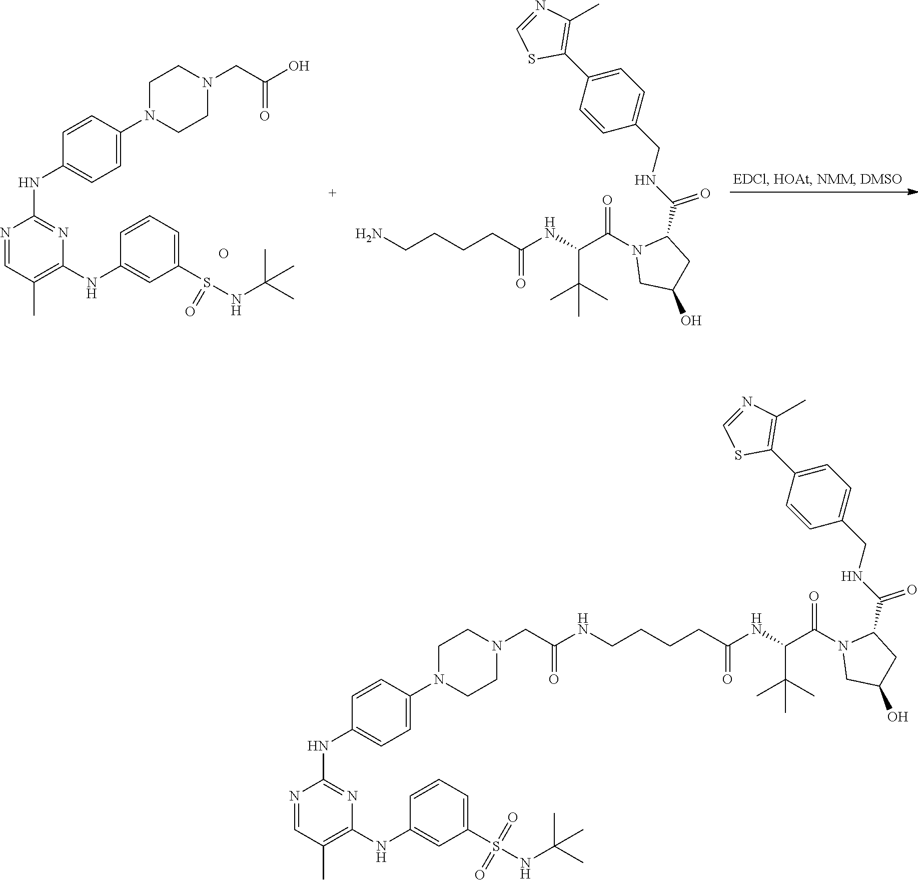

JA-100 was synthesized following the standard procedure for preparing JA-015 (4.2 mg, 22% yield). MS (ESI) m/z: 1066.9 [M+H]⁺.

### Example 189. (2S,4R)-1-((S)-2-(6-(2-(4-(4-((4-((3-(N-(tert-Butyl)sulfamoyl)phenyl)amino)-5-methylpyrimidin-2-yl)amino)phenyl)piperazin-1-yl)acetamido)hexanamido)-3,3-dimethylbutanoyl)-4-hydroxy-N-(4-(4-methylthiazol-5-yl)benzyl)pyrrolidine-2-carboxamide (JA-101)

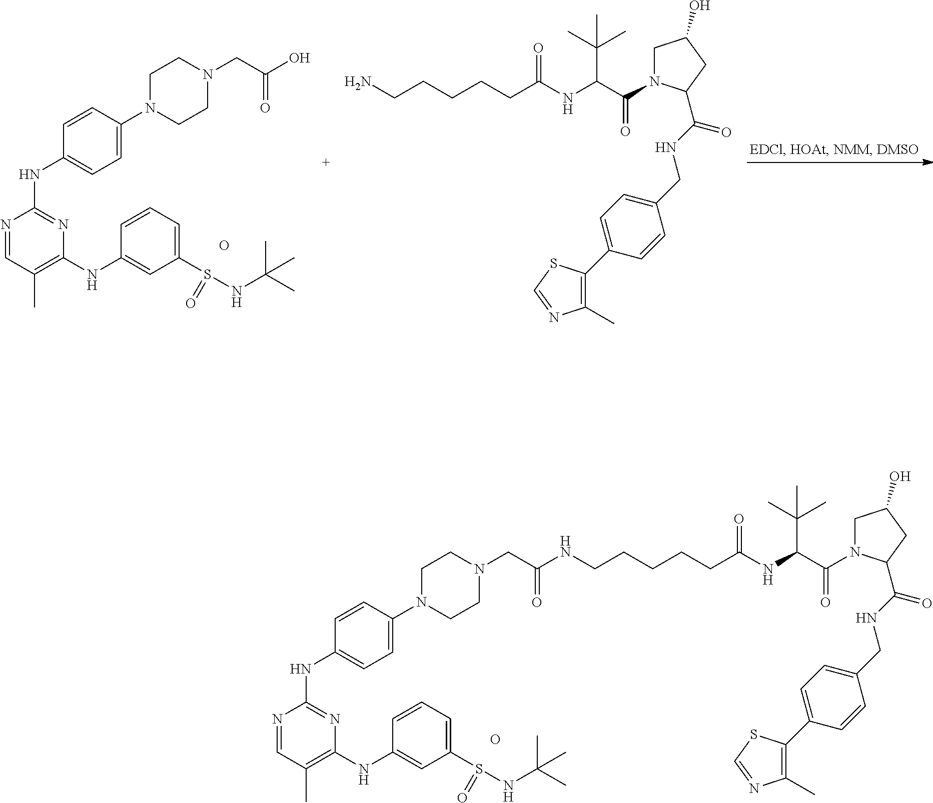

JA-101 was synthesized following the standard procedure for preparing JA-015 (8 mg, 41% yield). MS (ESI) m/z: 1079.8 [M+H]⁺.

### Example 190. (2S,4R)-1-((S)-2-(7-(2-(4-(4-((4-((3-(N-(tert-Butyl)sulfamoyl)phenyl)amino)-5-methylpyrimidin-2-yl)amino)phenyl)piperazin-1-yl)acetamido)heptanamido)-3,3-dimethylbutanoyl)-4-hydroxy-N-(4-(4-methylthiazol-5-yl)benzyl)pyrrolidine-2-carboxamide (JA-102)

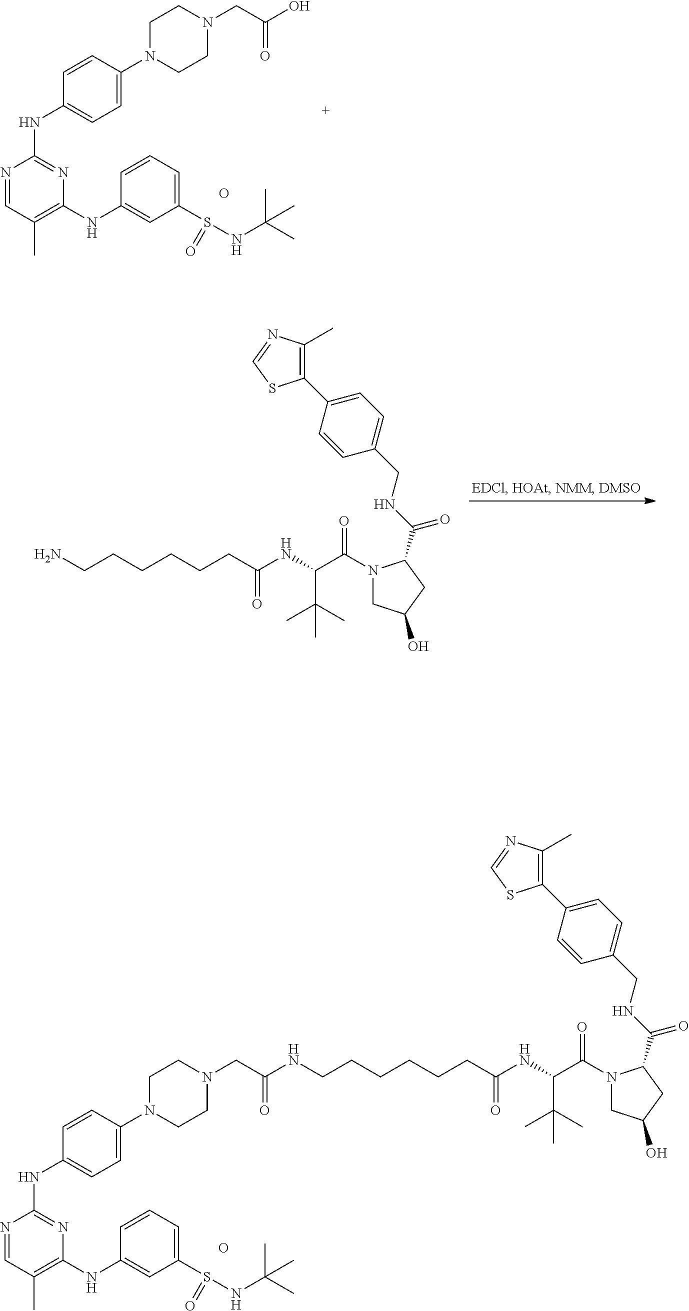

JA-102 was synthesized following the standard procedure for preparing JA-015 (1.04 mg, 5% yield). MS (ESI) m/z: 1094.2 [M+H]⁺.

### Example 191. (2S,4R)-1-((S)-2-(8-(2-(4-(4-((4-((3-(N-(tert-Butyl)sulfamoyl)phenyl)amino)-5-methylpyrimidin-2-yl)amino)phenyl)piperazin-1-yl)acetamido)octanamido)-3,3-dimethylbutanoyl)-4-hydroxy-N-(4-(4-methylthiazol-5-yl)benzyl)pyrrolidine-2-carboxamide (JA-103)

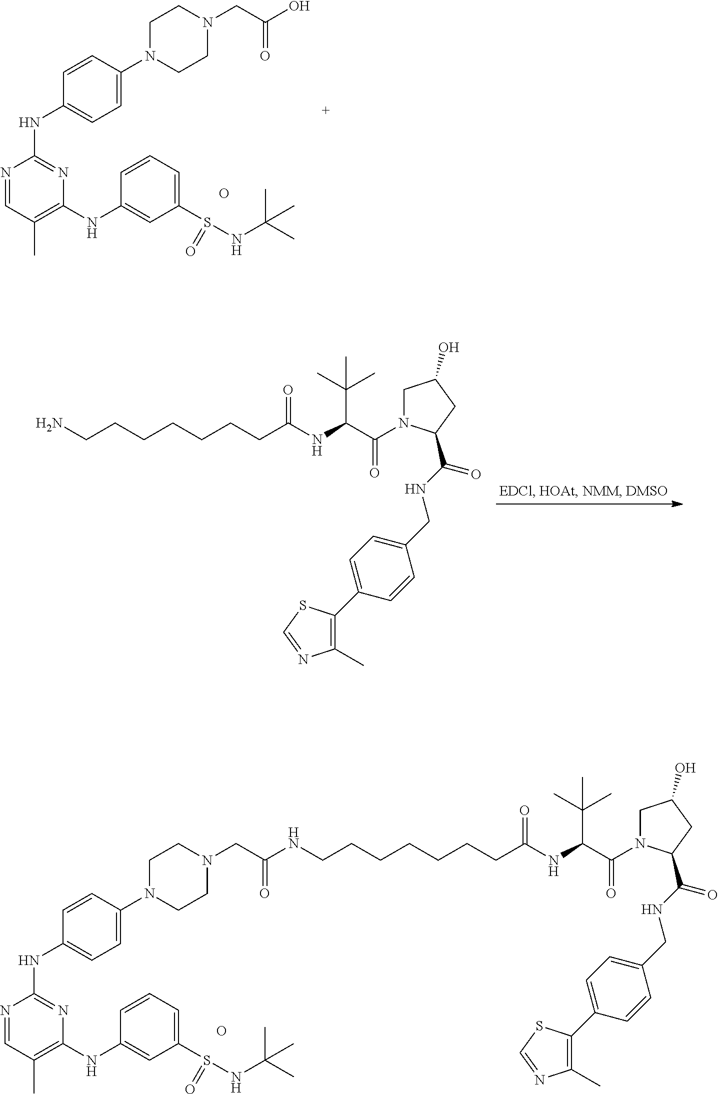

JA-103 was synthesized following the standard procedure for preparing JA-015 (3.7 mg, 19% yield). MS (ESI) m/z: 1108.1 [M+H]⁺.

### Example 192. (2S,4R)-1-((S)-2-(9-(2-(4-(4-((4-((3-(N-(tert-Butyl)sulfamoyl)phenyl)amino)-5-methylpyrimidin-2-yl)amino)phenyl)piperazin-1-yl)acetamido)nonanamido)-3,3-dimethylbutanoyl)-4-hydroxy-N-(4-(4-methylthiazol-5-yl)benzyl)pyrrolidine-2-carboxamide (JA-104)

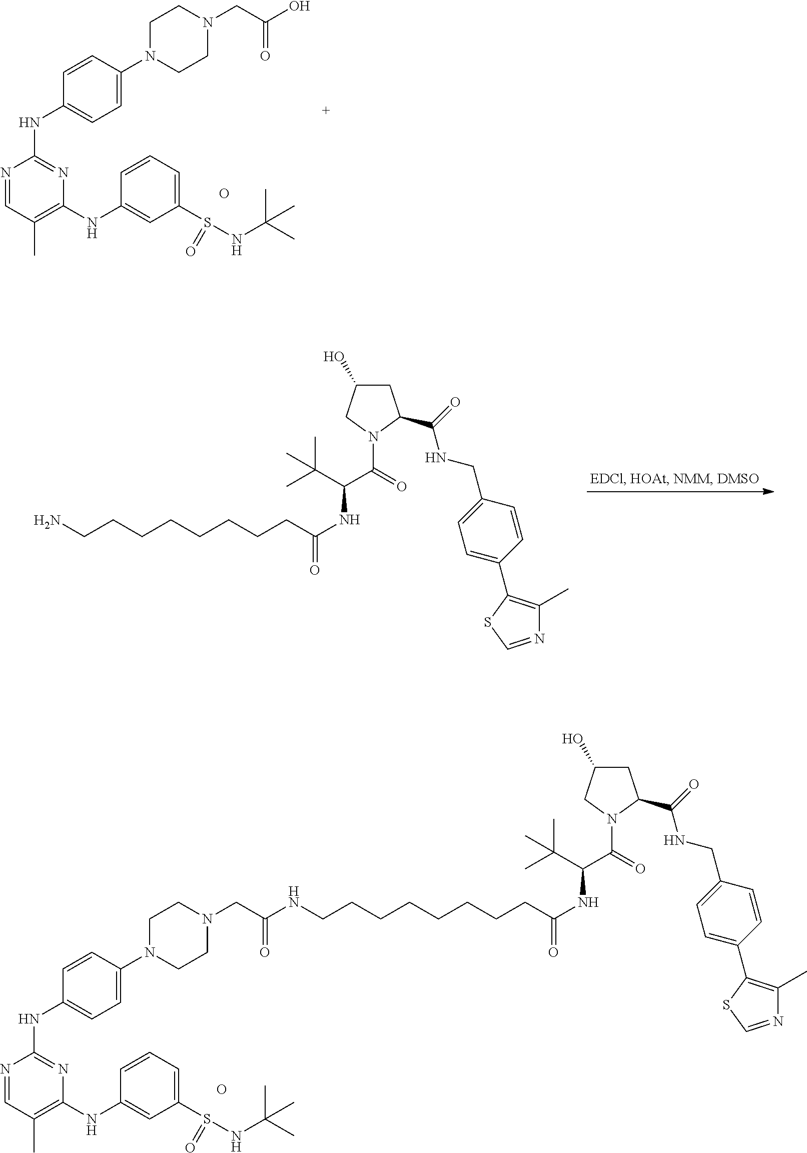

JA-104 was synthesized following the standard procedure for preparing JA-015 (6 mg, 30% yield). MS (ESI) m/z: 1122.4 [M+H]⁺.

### Example 193. (2S,4R)-1-((S)-2-(10-(2-(4-(4-((4-((3-(N-(tert-Butyl)sulfamoyl)phenyl)amino)-5-methylpyrimidin-2-yl)amino)phenyl)piperazin-1-yl)acetamido)decanamido)-3,3-dimethylbutanoyl)-4-hydroxy-N-(4-(4-methylthiazol-5-yl)benzyl)pyrrolidine-2-carboxamide (JA-105)

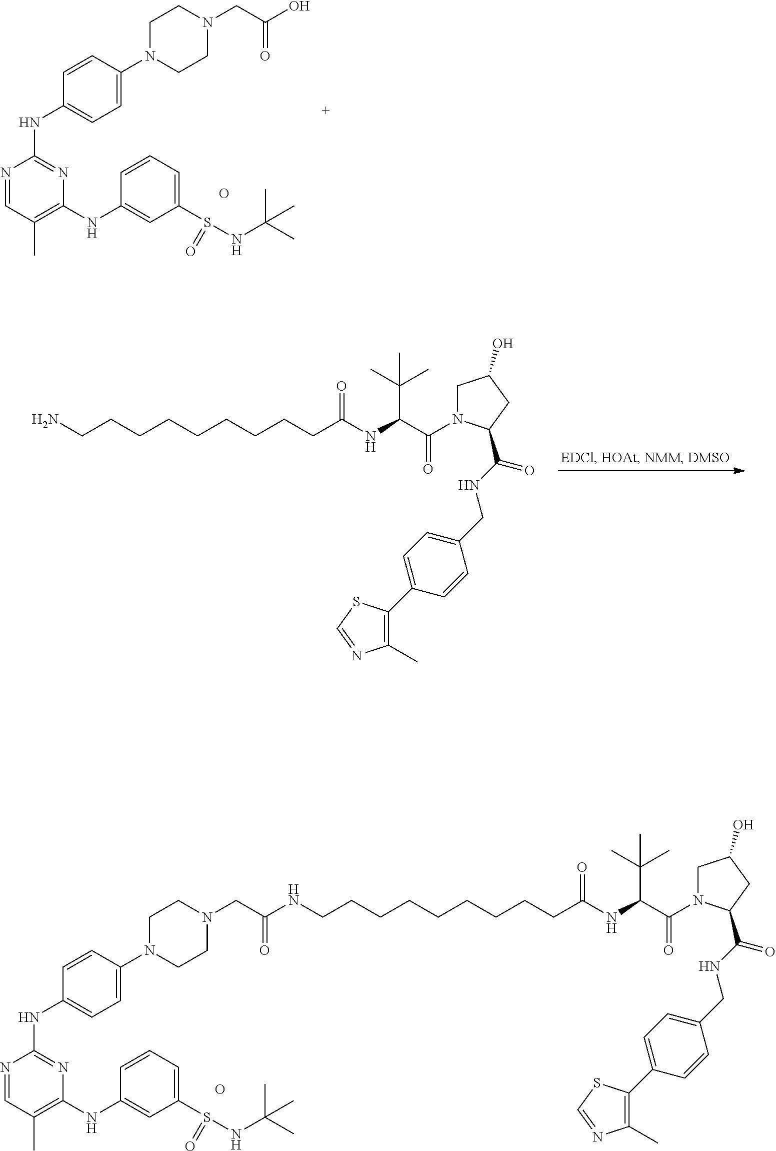

JA-105 was synthesized following the standard procedure for preparing JA-015 (9 mg, 44% yield). MS (ESI) m/z: 1136.2 [M+H]⁺.

### Example 194. (2S,4R)-1-((S)-2-(11-(2-(4-(4-((4-((3-(N-(tert-Butyl)sulfamoyl)phenyl)amino)-5-methylpyrimidin-2-yl)amino)phenyl)piperazin-1-yl)acetamido)undecanamido)-3,3-dimethylbutanoyl)-4-hydroxy-N-(4-(4-methylthiazol-5-yl)benzyl)pyrrolidine-2-carboxamide (JA-106)

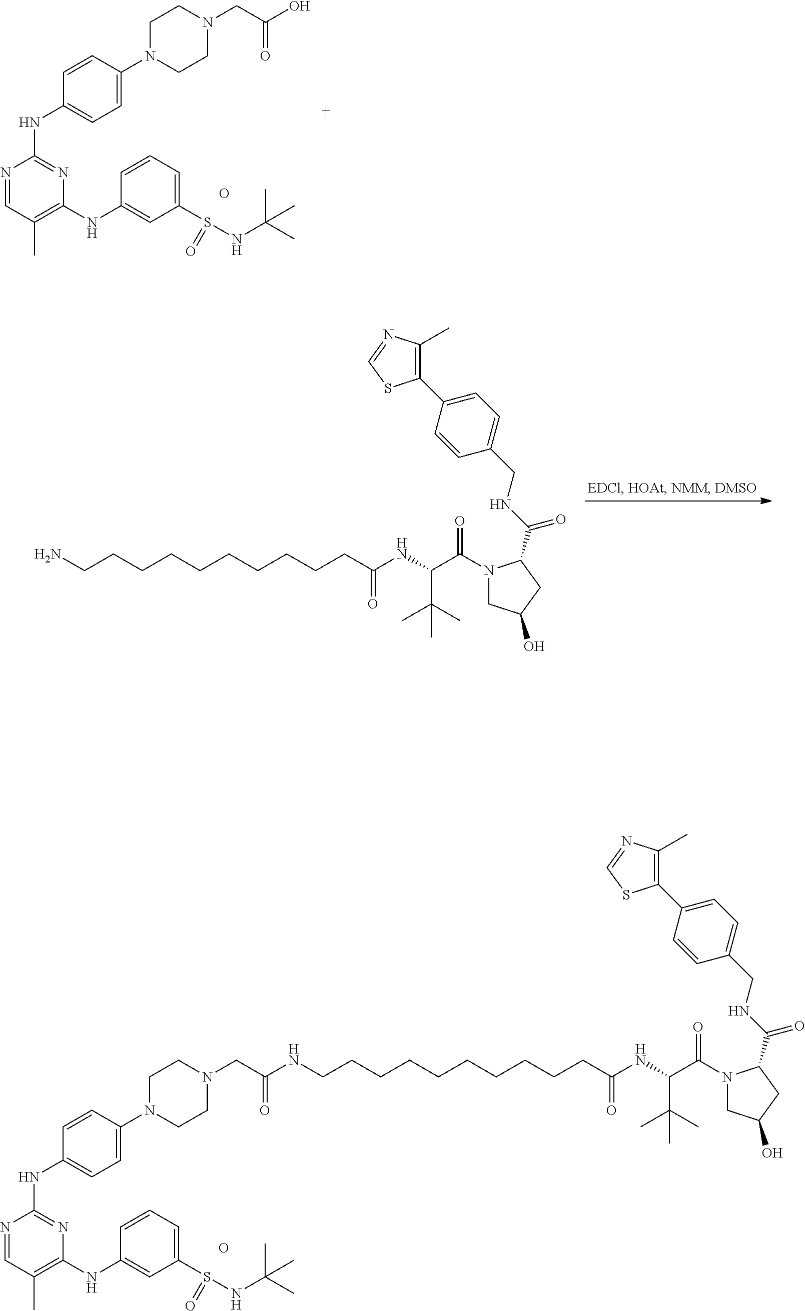

JA-106 was synthesized following the standard procedure for preparing JA-015 (11.4 mg, 55% yield). MS (ESI) m/z: 1150.8 [M+H]⁺.

### Example 195. N-(tert-Butyl)-3-((2-((4-(4-(5-((2-(2,6-dioxopiperidin-3-yl)-1,3-dioxoisoindolin-4-yl)amino)pentanoyl)piperazin-1-yl)phenyl)amino)-5-methylpyrimidin-4-yl)amino)benzenesulfonamide (JA-107)

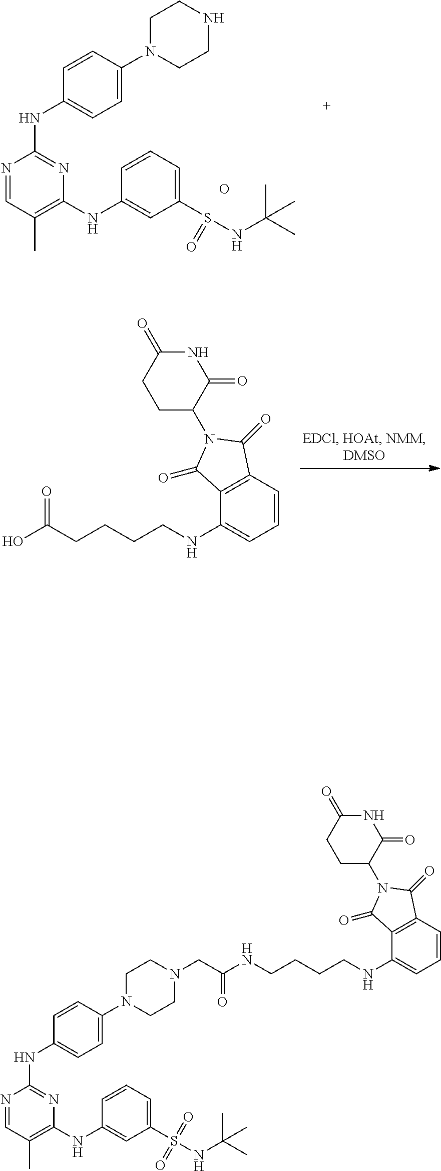

JA-107 was synthesized following the standard procedure for preparing JA-015 (6 mg, 35% yield). MS (ESI) m/z: 851.9 [M+H]⁺.

### Example 196. (2S,4R)-1-((S)-2-(tert-Butyl)-14-(4-(4-((4-((3-(N-(tert-butyl)sulfamoyl)phenyl)amino)-5-methylpyrimidin-2-yl)amino)phenyl)piperazin-1-yl)-4,14-dioxo-6,9,12-trioxa-3-azatetradecanoyl)-4-hydroxy-N-(4-(4-methylthiazol-5-yl)benzyl)pyrrolidine-2-carboxamide (JA-108)

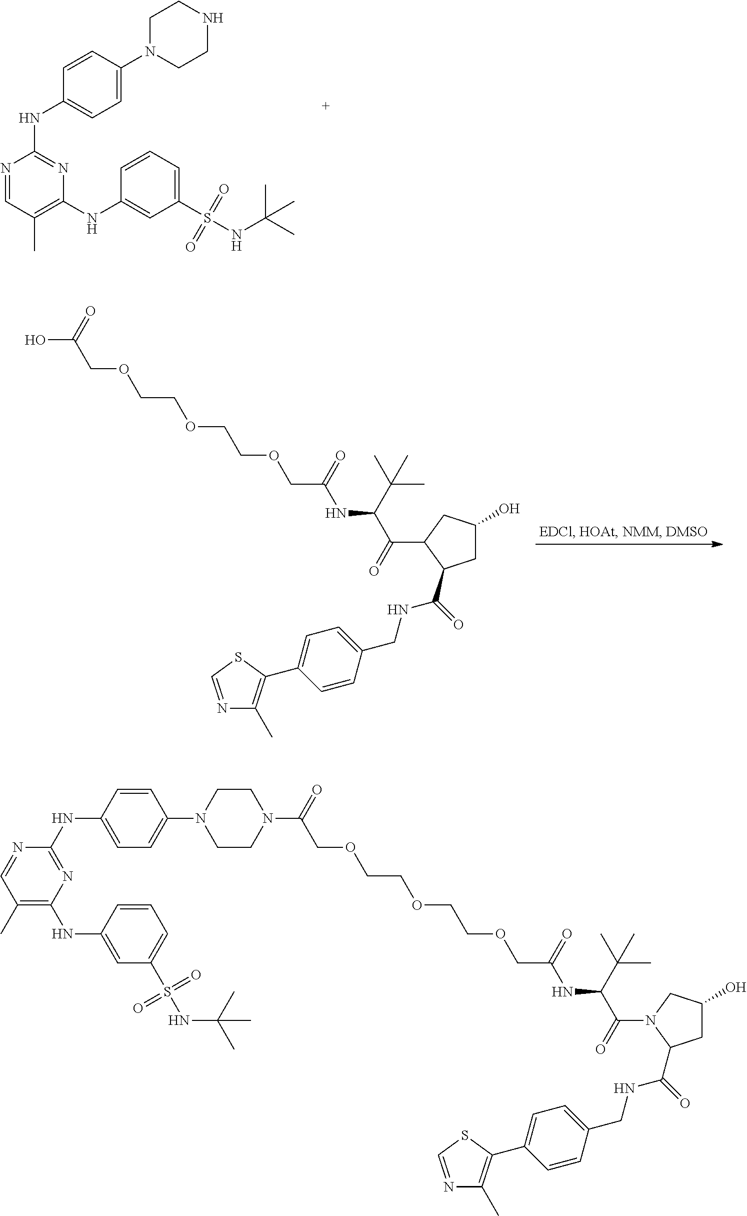 [SMILES: Cc1cnc(Nc2ccc(N3CCNCC3)cc2)nc1Nc1cccc(S(=O)(=O)NC(C)(C)C)c1 ; Cc1ncsc1-c1ccc(CNC(=O)[C@@H]2C[C@@H](O)CC2C(=O)[C@@H](NC(=O)COCCOCCOCC(=O)O)C(C)(C)C)cc1 ; Cc1cnc(Nc2ccc(N3CCN(C(=O)COCCOCCOCC(=O)N[C@H](C(=O)N4C[C@H](O)CC4C(=O)NCc4ccc(-c5scnc5C)cc4)C(C)(C)C)CC3)cc2)nc1Nc1cccc(S(=O)(=O)NC(C)(C)C)c1]

JA-108 was synthesized following the standard procedure for preparing JA-015 (6 mg, 27% yield). MS (ESI) m/z: 1113.0 [M+H]⁺.

### Example 197. N-(4-((2-(2,6-Dioxopiperidin-3-yl)-1,3-dioxoisoindolin-4-yl)amino)butyl)-2-(4-(4-((5-(4-(methylsulfonyl)phenyl)-[1,2,4]triazolo[1,5-a]pyridin-2-yl)amino)phenyl)piperazin-1-yl)acetamide (JA-109)

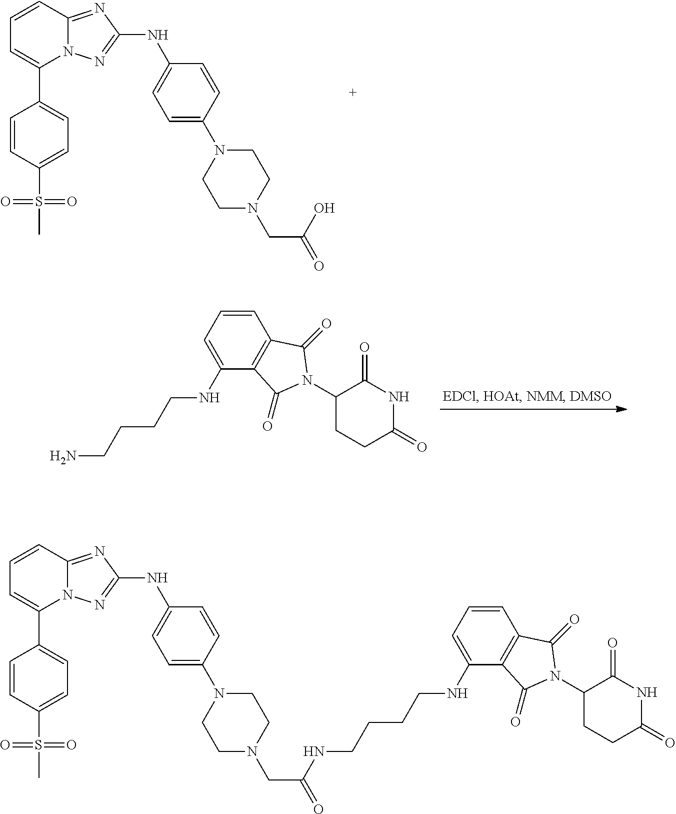 [SMILES: CS(=O)(=O)c1ccc(-c2cccc3nc(Nc4ccc(N5CCN(CC(=O)O)CC5)cc4)nn23)cc1 ; NCCCCNc1cccc2c1C(=O)N(C1CCC(=O)NC1=O)C2=O ; CS(=O)(=O)c1ccc(-c2cccc3nc(Nc4ccc(N5CCN(CC(=O)NCCCCNc6cccc7c6C(=O)N(C6CCC(=O)NC6=O)C7=O)CC5)cc4)nn23)cc1]

JA-109 was synthesized following the standard procedure for preparing JA-010 (7.2 mg, 61% yield). MS (ESI) m/z: 833.3 [M+H]⁺.

### Example 198. N-(2-(2-((2-(2,6-Dioxopiperidin-3-yl)-1,3-dioxoisoindolin-4-yl)amino)ethoxy)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-110)

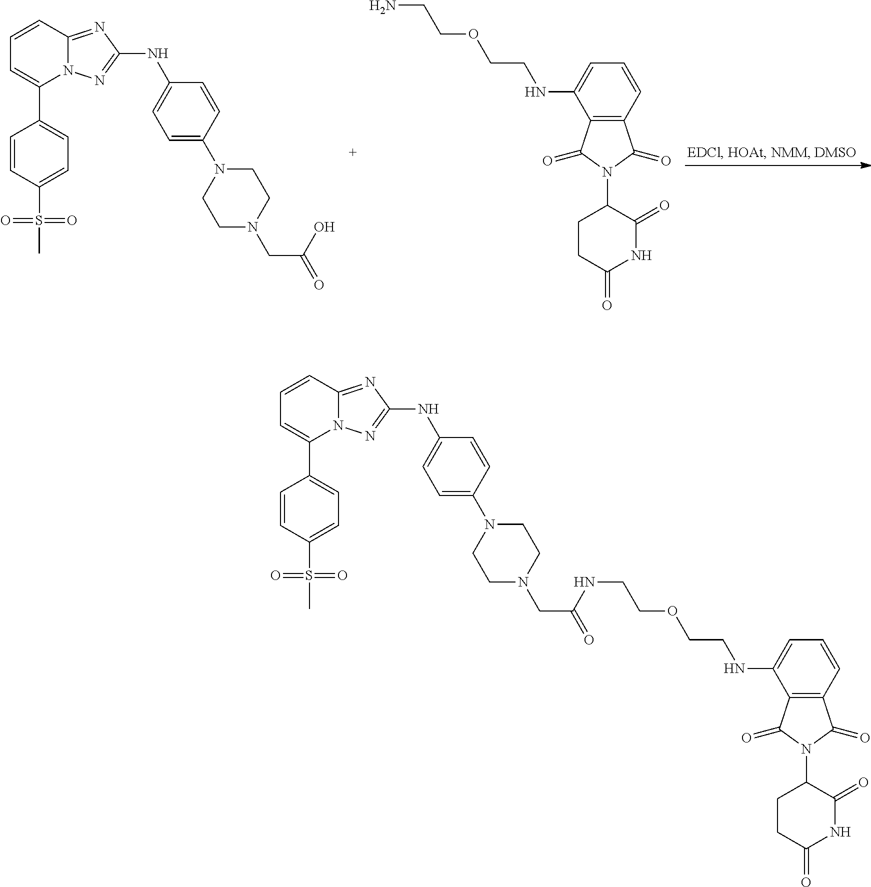 [SMILES: CS(=O)(=O)c1ccc(-c2cccc3nc(Nc4ccc(N5CCN(CC(=O)O)CC5)cc4)nn23)cc1 ; NCCOCCNc1cccc2c1C(=O)N(C1CCC(=O)NC1=O)C2=O ; CS(=O)(=O)c1ccc(-c2cccc3nc(Nc4ccc(N5CCN(CC(=O)NCCOCCNc6cccc7c6C(=O)N(C6CCC(=O)NC6=O)C7=O)CC5)cc4)nn23)cc1]

JA-110 was synthesized following the standard procedure for preparing JA-010 (6.7 mg, 62% yield). MS (ESI) m/z: 849.3 [M+H]⁺.

### Example 199. N-(3-((2-(2,6-Dioxopiperidin-3-yl)-1,3-dioxoisoindolin-4-yl)amino)propyl)-2-(4-(4-((5-(4-(methylsulfonyl)phenyl)-[1,2,4]triazolo[1,5-a]pyridin-2-yl)amino)phenyl)piperazin-1-yl)acetamide (JA-111)

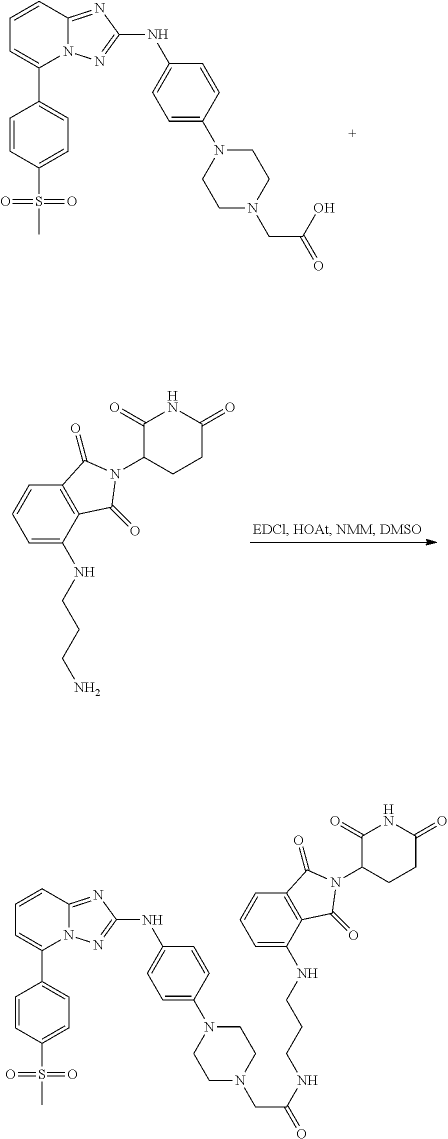 [SMILES: CS(=O)(=O)c1ccc(-c2cccc3nc(Nc4ccc(N5CCN(CC(=O)O)CC5)cc4)nn23)cc1 ; NCCCNc1cccc2c1C(=O)N(C1CCC(=O)NC1=O)C2=O ; CS(=O)(=O)c1ccc(-c2cccc3nc(Nc4ccc(N5CCN(CC(=O)NCCCNc6cccc7c6C(=O)N(C6CCC(=O)NC6=O)C7=O)CC5)cc4)nn23)cc1]

JA-111 was synthesized following the standard procedure for preparing JA-010 (7.0 mg, 70% yield). MS (ESI) m/z: 819.3 [M+H]⁺.

### Example 200. N-(6-((2-(2,6-Dioxopiperidin-3-yl)-1,3-dioxoisoindolin-4-yl)amino)hexyl)-2-(4-(4-((5-(4-(methylsulfonyl)phenyl)-[1,2,4]triazolo[1,5-a]pyridin-2-yl)amino)phenyl)piperazin-1-yl)acetamide (JA-112)

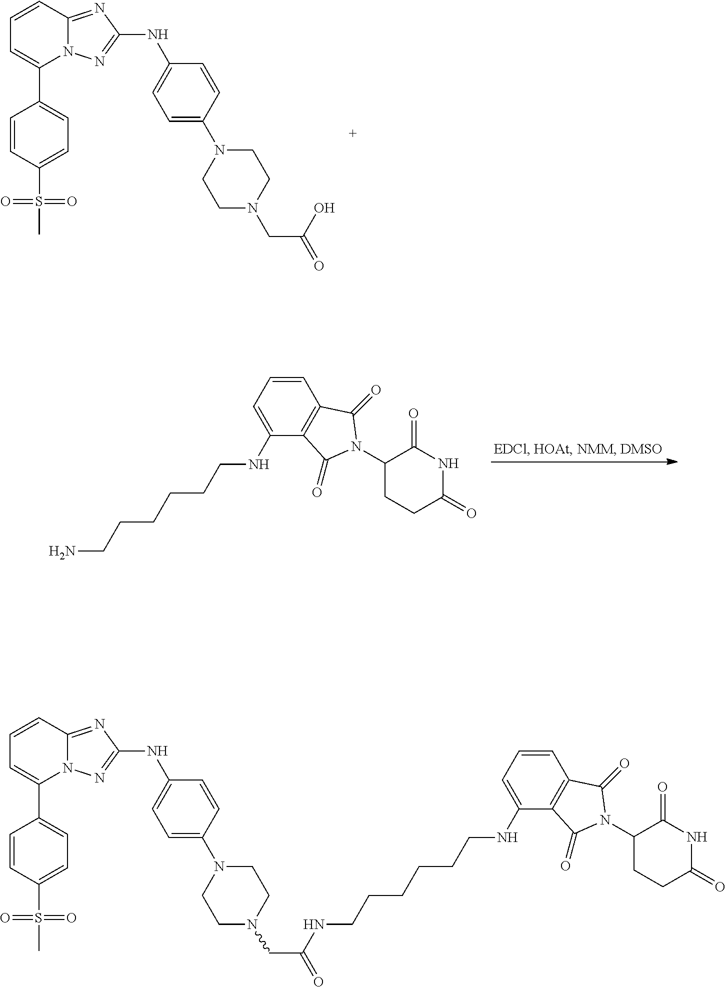 [SMILES: CS(=O)(=O)c1ccc(-c2cccc3nc(Nc4ccc(N5CCN(CC(=O)O)CC5)cc4)nn23)cc1 ; NCCCCCCNc1cccc2c1C(=O)N(C1CCC(=O)NC1=O)C2=O ; CS(=O)(=O)c1ccc(-c2cccc3nc(Nc4ccc(N5CCN(CC(=O)NCCCCCCNc6cccc7c6C(=O)N(C6CCC(=O)NC6=O)C7=O)CC5)cc4)nn23)cc1]

JA-112 was synthesized following the standard procedure for preparing JA-010 (7.1 mg, 71% yield). MS (ESI) m/z: 861.3 [M+H]⁺.

### Example 201. N-(5-((2-(2,6-Dioxopiperidin-3-yl)-1,3-dioxoisoindolin-4-yl)amino)pentyl)-2-(4-(4-((5-(4-(methylsulfonyl)phenyl)-[1,2,4]triazolo[1,5-a]pyridin-2-yl)amino)phenyl)piperazin-1-yl)acetamide (JA-113)

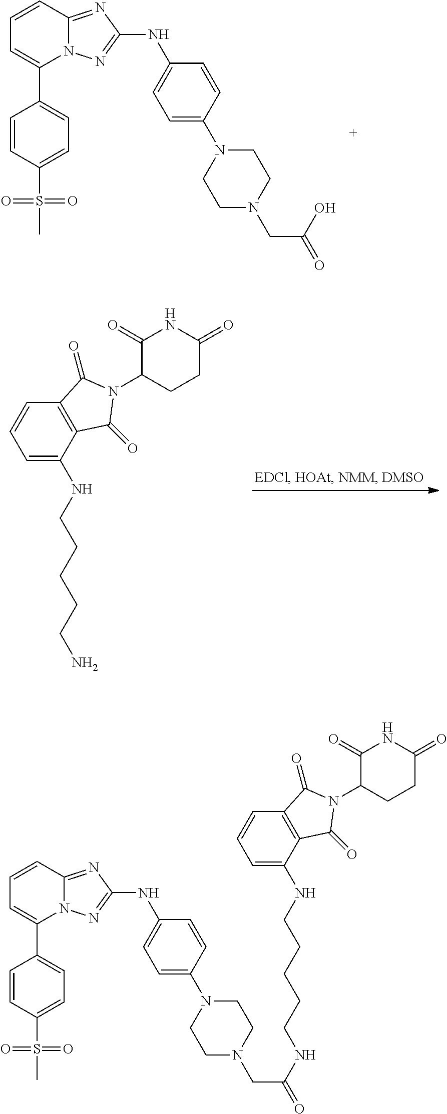 [SMILES: CS(=O)(=O)c1ccc(-c2cccc3nc(Nc4ccc(N5CCN(CC(=O)O)CC5)cc4)nn23)cc1 ; NCCCCCNc1cccc2c1C(=O)N(C1CCC(=O)NC1=O)C2=O ; CS(=O)(=O)c1ccc(-c2cccc3nc(Nc4ccc(N5CCN(CC(=O)NCCCCCNc6cccc7c6C(=O)N(C6CCC(=O)NC6=O)C7=O)CC5)cc4)nn23)cc1]

JA-113 was synthesized following the standard procedure for preparing JA-010 (6.7 mg, 72% yield). MS (ESI) m/z: 847.3 [M+H]⁺.

### Example 202. N-(14-((2-(2,6-Dioxopiperidin-3-yl)-1,3-dioxoisoindolin-4-yl)amino)-3,6,9,12-tetraoxatetradecyl)-2-(4-(4-((5-(4-(methylsulfonyl)phenyl)-[1,2,4]triazolo[1,5-a]pyridin-2-yl)amino)phenyl)piperazin-1-yl)acetamide (JA-114)

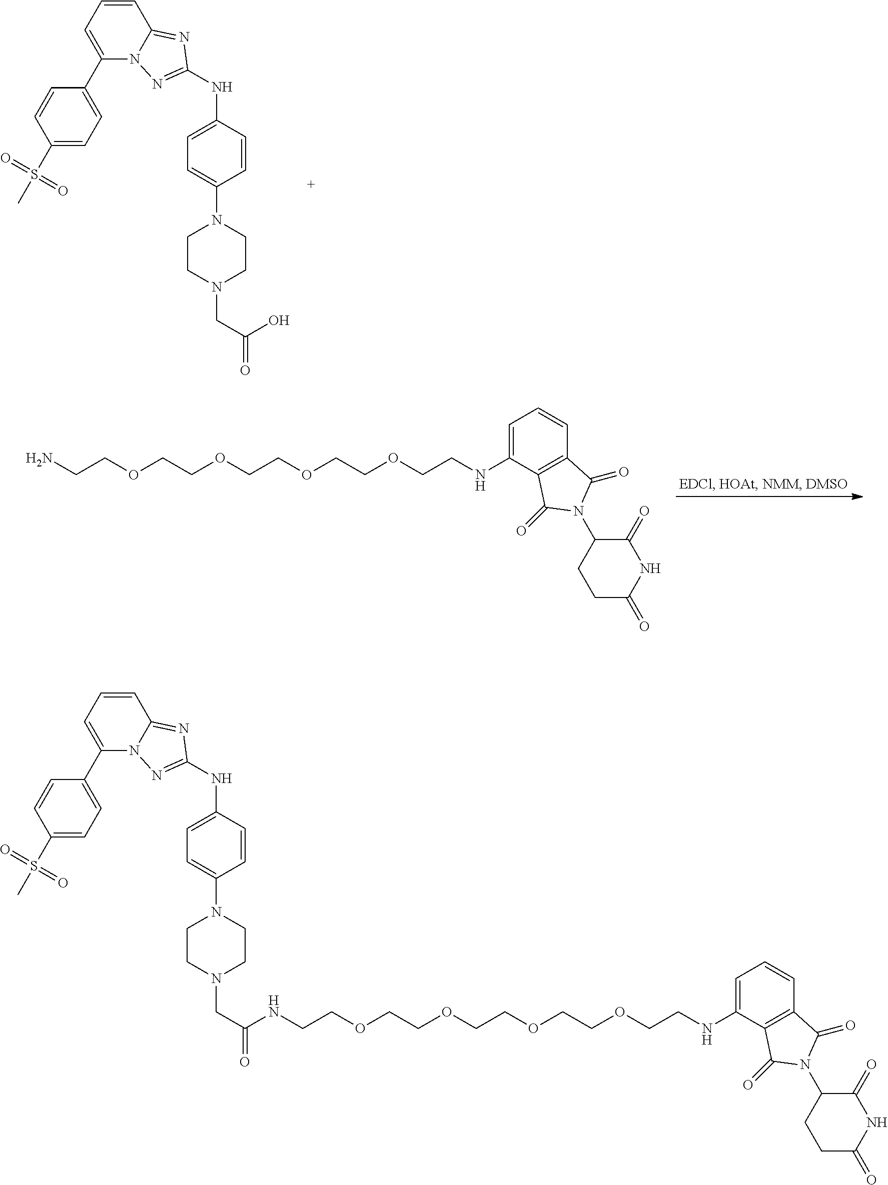 [SMILES: CS(=O)(=O)c1ccc(-c2cccc3nc(Nc4ccc(N5CCN(CC(=O)O)CC5)cc4)nn23)cc1 ; NCCOCCOCCOCCOCCNc1cccc2c1C(=O)N(C1CCC(=O)NC1=O)C2=O ; CS(=O)(=O)c1ccc(-c2cccc3nc(Nc4ccc(N5CCN(CC(=O)NCCOCCOCCOCCOCCNc6cccc7c6C(=O)N(C6CCC(=O)NC6=O)C7=O)CC5)cc4)nn23)cc1]

JA-114 was synthesized following the standard procedure for preparing JA-010 (7.5 mg, 71% yield). MS (ESI) m/z: 982.4 [M+H]⁺.

### Example 203. N-(2-(2-(2-((2-(2,6-Dioxopiperidin-3-yl)-1,3-dioxoisoindolin-4-yl)amino)ethoxy)ethoxy)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-115)

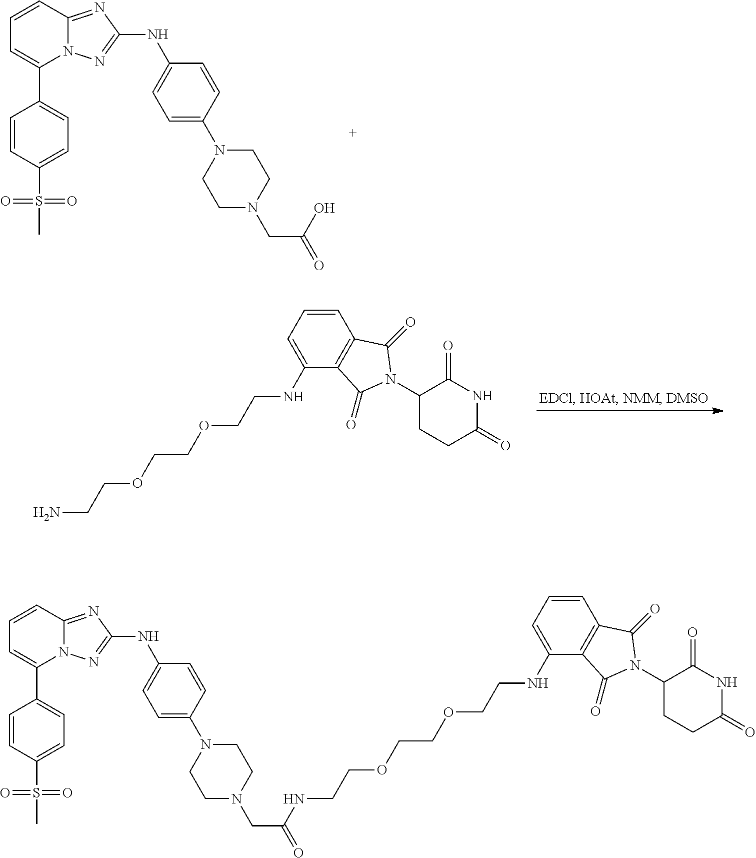 [SMILES: CS(=O)(=O)c1ccc(-c2cccc3nc(Nc4ccc(N5CCN(CC(=O)O)CC5)cc4)nn23)cc1 ; NCCOCCOCCNc1cccc2c1C(=O)N(C1CCC(=O)NC1=O)C2=O ; CS(=O)(=O)c1ccc(-c2cccc3nc(Nc4ccc(N5CCN(CC(=O)NCCOCCOCCNc6cccc7c6C(=O)N(C6CCC(=O)NC6=O)C7=O)CC5)cc4)nn23)cc1]

JA-115 was synthesized following the standard procedure for preparing JA-010 (6.5 mg, 72% yield). MS (ESI) m/z: 893.3 [M+H]⁺.

### Example 204. N-(7-((2-(2,6-Dioxopiperidin-3-yl)-1,3-dioxoisoindolin-4-yl)amino)heptyl)-2-(4-(4-((5-(4-(methylsulfonyl)phenyl)-[1,2,4]triazolo[1,5-a]pyridin-2-yl)amino)phenyl)piperazin-1-yl)acetamide (JA-116)

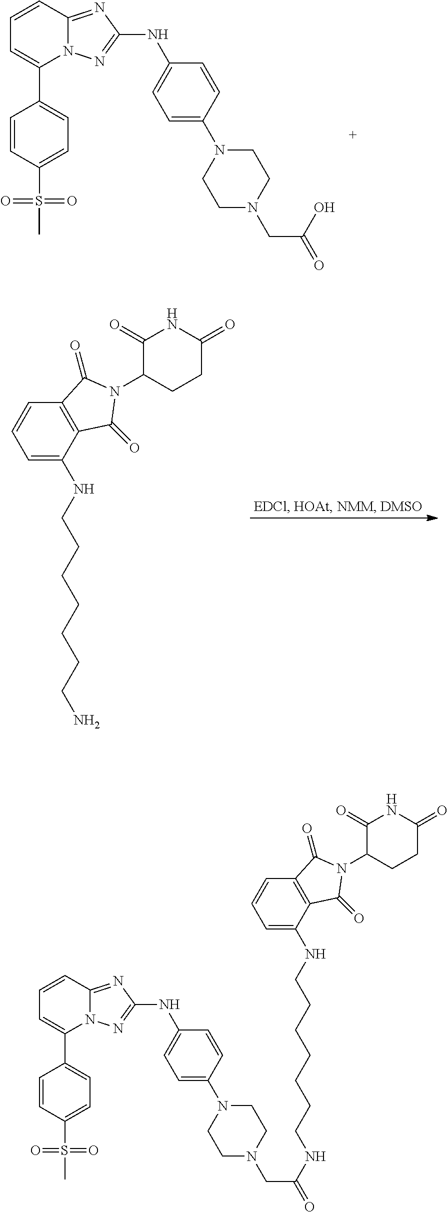 [SMILES: CS(=O)(=O)c1ccc(-c2cccc3nc(Nc4ccc(N5CCN(CC(=O)O)CC5)cc4)nn23)cc1 ; NCCCCCCCNc1cccc2c1C(=O)N(C1CCC(=O)NC1=O)C2=O ; CS(=O)(=O)c1ccc(-c2cccc3nc(Nc4ccc(N5CCN(CC(=O)NCCCCCCCNc6cccc7c6C(=O)N(C6CCC(=O)NC6=O)C7=O)CC5)cc4)nn23)cc1]

JA-116 was synthesized following the standard procedure for preparing JA-010 (6.5 mg, 71% yield). MS (ESI) m/z: 875.4 [M+H]⁺.

### Example 205. N-(2-((2-(2,6-Dioxopiperidin-3-yl)-1,3-dioxoisoindolin-4-yl)amino)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-117)

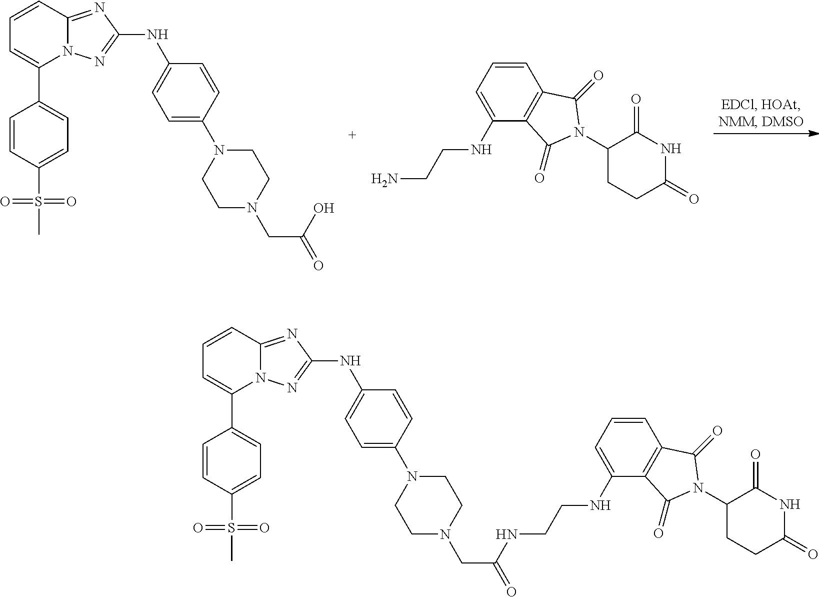 [SMILES: CS(=O)(=O)c1ccc(-c2cccc3nc(Nc4ccc(N5CCN(CC(=O)O)CC5)cc4)nn23)cc1 ; NCCNc1cccc2c1C(=O)N(C1CCC(=O)NC1=O)C2=O ; CS(=O)(=O)c1ccc(-c2cccc3nc(Nc4ccc(N5CCN(CC(=O)NCCNc6cccc7c6C(=O)N(C6CCC(=O)NC6=O)C7=O)CC5)cc4)nn23)cc1]

JA-117 was synthesized following the standard procedure for preparing JA-010 (6.3 mg, 75% yield). MS (ESI) m/z: 805.3 [M+H]⁺.

### Example 206. N-(8-((2-(2,6-Dioxopiperidin-3-yl)-1,3-dioxoisoindolin-4-yl)amino)octyl)-2-(4-(4-((5-(4-(methylsulfonyl)phenyl)-[1,2,4]triazolo[1,5-a]pyridin-2-yl)amino)phenyl)piperazin-1-yl)acetamide (JA-118)

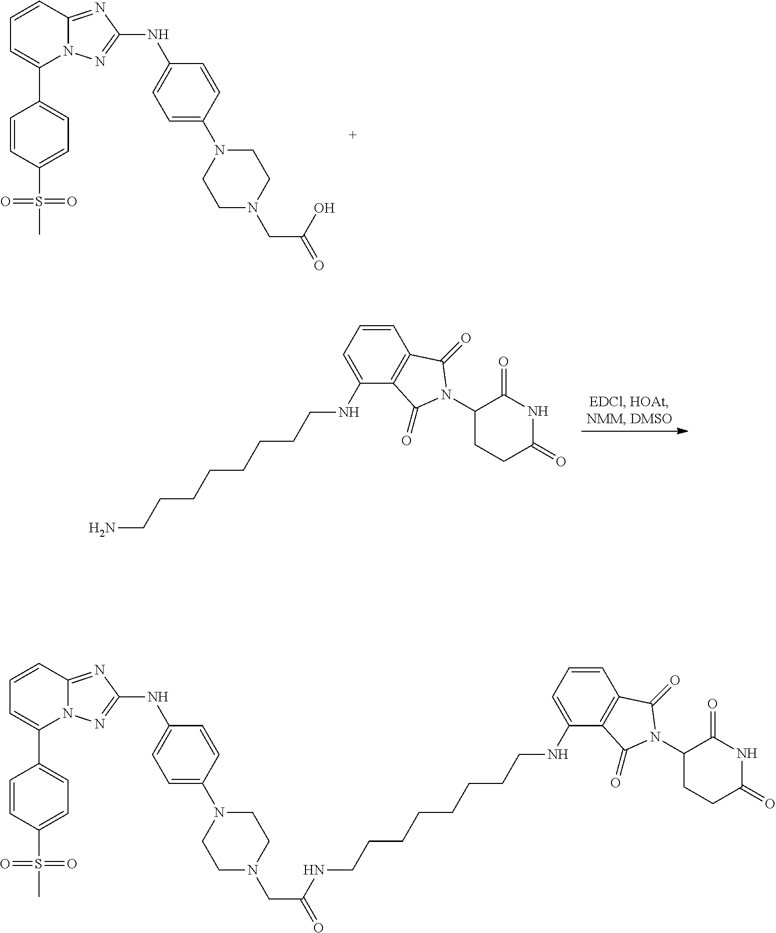 [SMILES: CS(=O)(=O)c1ccc(-c2cccc3nc(Nc4ccc(N5CCN(CC(=O)O)CC5)cc4)nn23)cc1 ; NCCCCCCCCNc1cccc2c1C(=O)N(C1CCC(=O)NC1=O)C2=O ; CS(=O)(=O)c1ccc(-c2cccc3nc(Nc4ccc(N5CCN(CC(=O)NCCCCCCCCNc6cccc7c6C(=O)N(C6CCC(=O)NC6=O)C7=O)CC5)cc4)nn23)cc1]

JA-118 was synthesized following the standard procedure for preparing JA-010 (7.5 mg, 76% yield). MS (ESI) m/z: 889.4 [M+H]⁺.

### Example 207. (2S,4R)-1-((S)-3,3-Dimethyl-2-(3-(2-(4-(4-((5-(4-(methylsulfonyl)phenyl)-[1,2,4]triazolo[1,5-a]pyridin-2-yl)amino)phenyl)piperazin-1-yl)acetamido)propanamido)butanoyl)-4-hydroxy-N-(4-(4-methylthiazol-5-yl)benzyl)pyrrolidine-2-carboxamide (JA-119)

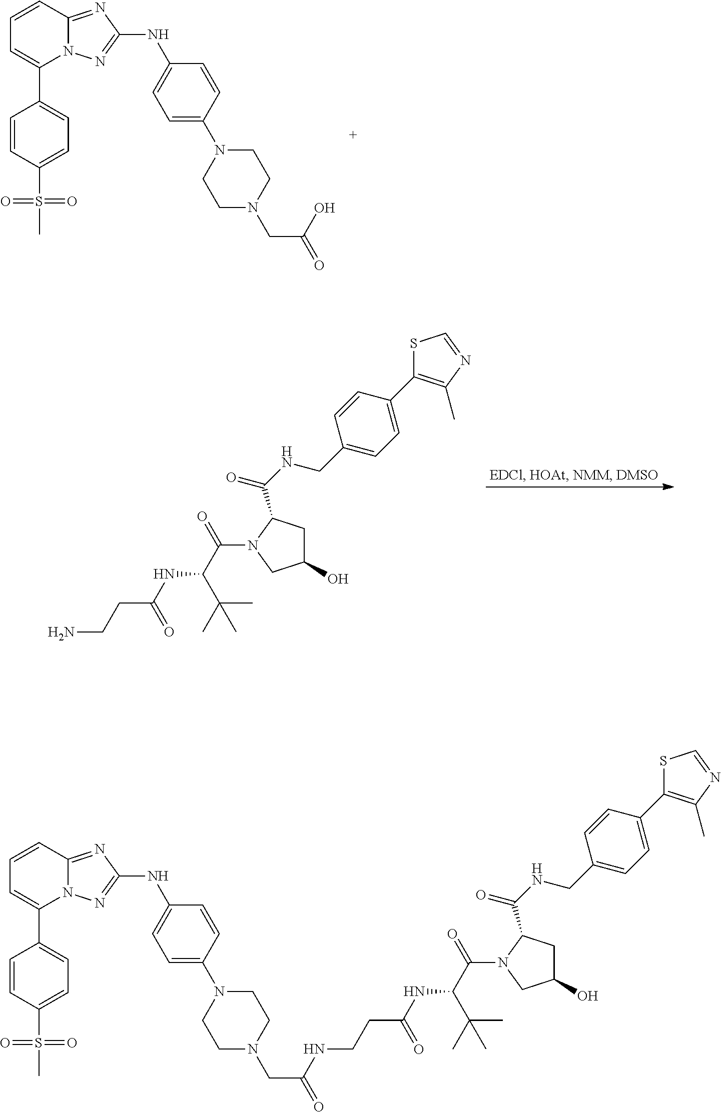 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCN)C(C)(C)C)cc1 ; Cc1ncsc1-c1ccc(CNC(=O)[C@@H]2C[C@@H](O)CN2C(=O)[C@@H](NC(=O)CCNC(=O)CN2CCN(c3ccc(Nc4nc5cccc(-c6ccc(S(C)(=O)=O)cc6)n5n4)cc3)CC2)C(C)(C)C)cc1]

JA-119 was synthesized following the standard procedure for preparing JA-010 (6.3 mg, 60% yield). MS (ESI) m/z: 989.4 [M+H]⁺.

### Example 208. (2S,4R)-1-((S)-3,3-Dimethyl-2-(10-(2-(4-(4-((5-(4-(methylsulfonyl)phenyl)-[1,2,4]triazolo[1,5-a]pyridin-2-yl)amino)phenyl)piperazin-1-yl)acetamido)decanamido)butanoyl)-4-hydroxy-N-(4-(4-methylthiazol-5-yl)benzyl)pyrrolidine-2-carboxamide (JA-120)

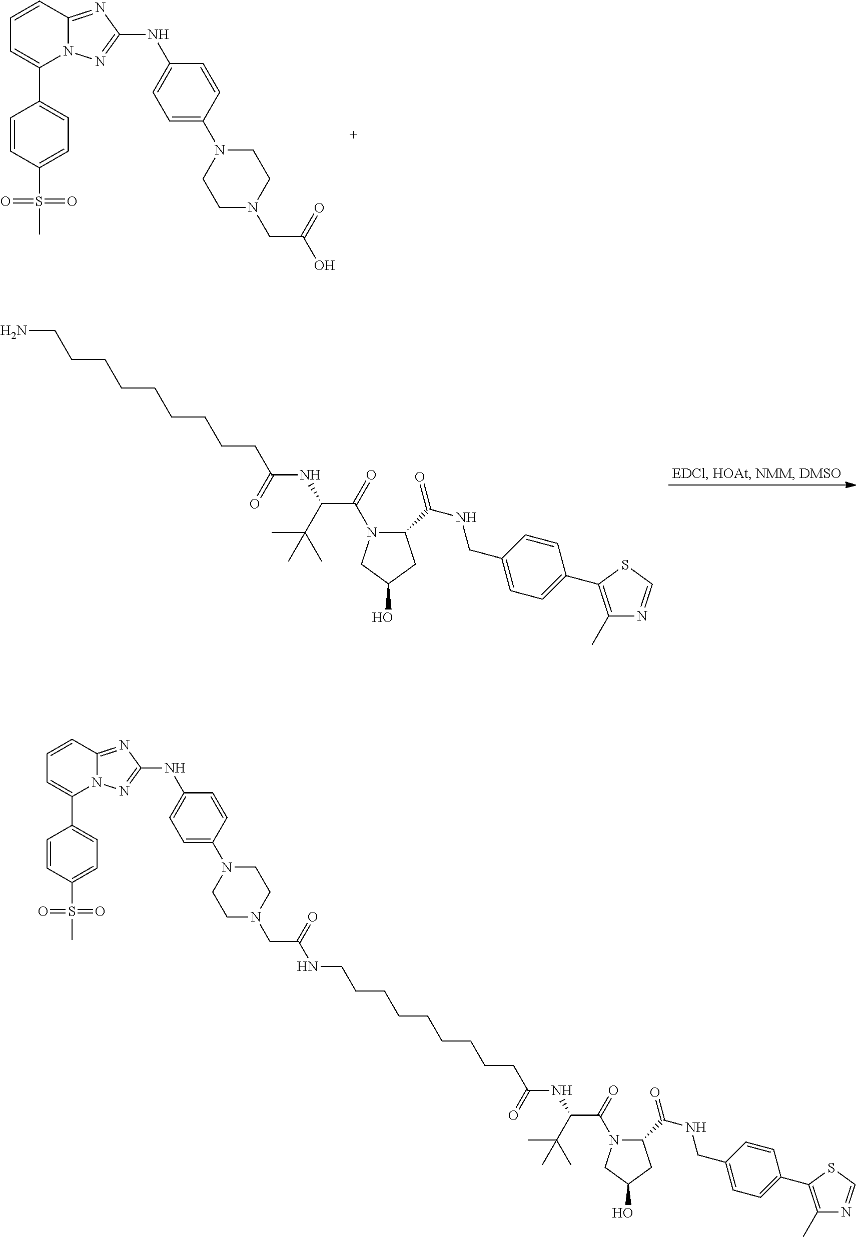 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCCCCCCN)C(C)(C)C)cc1 ; Cc1ncsc1-c1ccc(CNC(=O)[C@@H]2C[C@@H](O)CN2C(=O)[C@@H](NC(=O)CCCCCCCCCNC(=O)CN2CCN(c3ccc(Nc4nc5cccc(-c6ccc(S(C)(=O)=O)cc6)n5n4)cc3)CC2)C(C)(C)C)cc1]

JA-120 was synthesized following the standard procedure for preparing JA-010 (5.3 mg, 61% yield). MS (ESI) m/z: 1088.5 [M+H]⁺.

### Example 209. (2S,4R)-1-((S)-3,3-Dimethyl-2-(9-(2-(4-(4-((5-(4-(methylsulfonyl)phenyl)-[1,2,4]triazolo[1,5-a]pyridin-2-yl)amino)phenyl)piperazin-1-yl)acetamido)nonanamido)butanoyl)-4-hydroxy-N-(4-(4-methylthiazol-5-yl)benzyl)pyrrolidine-2-carboxamide (JA-121)

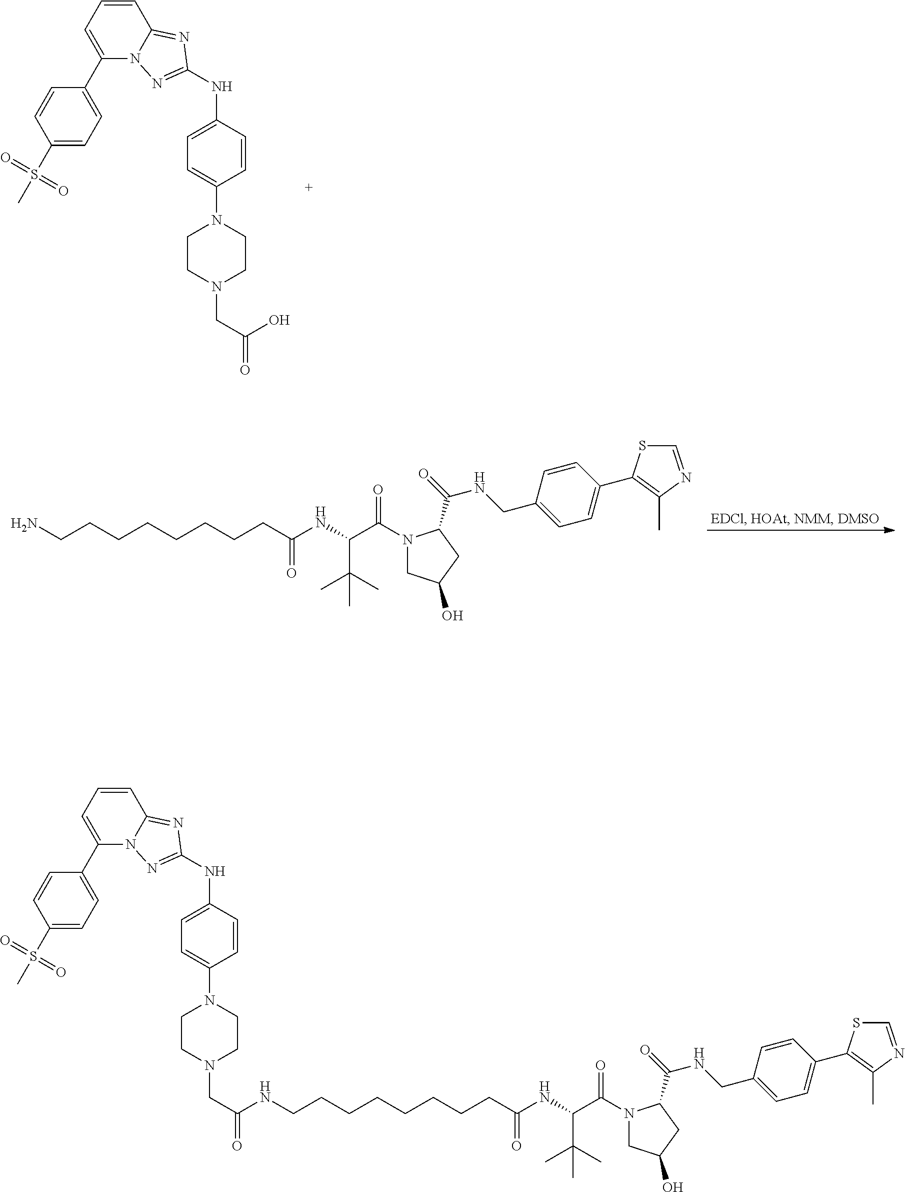 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCCCCCN)C(C)(C)C)cc1 ; Cc1ncsc1-c1ccc(CNC(=O)[C@@H]2C[C@@H](O)CN2C(=O)[C@@H](NC(=O)CCCCCCCCNC(=O)CN2CCN(c3ccc(Nc4nc5cccc(-c6ccc(S(C)(=O)=O)cc6)n5n4)cc3)CC2)C(C)(C)C)cc1]

JA-121 was synthesized following the standard procedure for preparing JA-010 (5.3 mg, 62% yield). MS (ESI) m/z: 1074.5 [M+H]⁺.

### Example 210. (2S,4R)-1-((S)-3,3-Dimethyl-2-(6-(2-(4-(4-((5-(4-(methylsulfonyl)phenyl)-[1,2,4]triazolo[1,5-a]pyridin-2-yl)amino)phenyl)piperazin-1-yl)acetamido)hexanamido)butanoyl)-4-hydroxy-N-(4-(4-methylthiazol-5-yl)benzyl)pyrrolidine-2-carboxamide (JA-122)

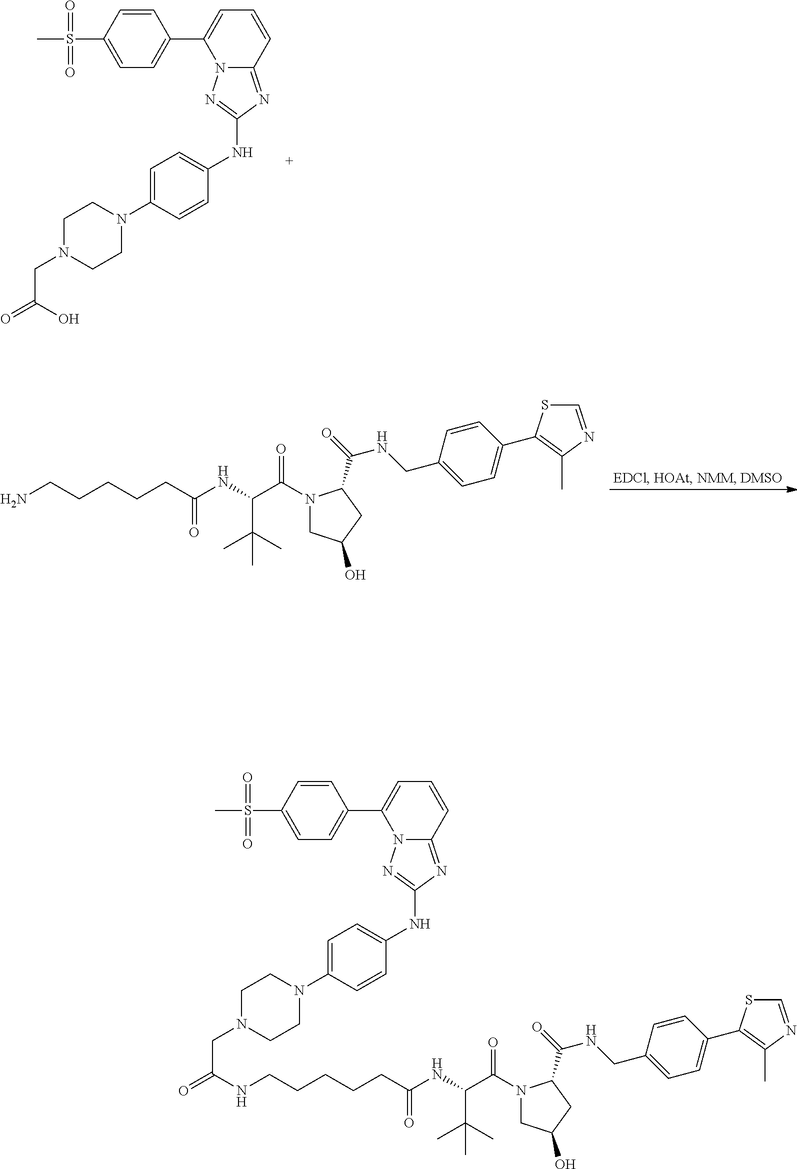 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCCN)C(C)(C)C)cc1 ; Cc1ncsc1-c1ccc(CNC(=O)[C@@H]2C[C@@H](O)CN2C(=O)[C@@H](NC(=O)CCCCCNC(=O)CN2CCN(c3ccc(Nc4nc5cccc(-c6ccc(S(C)(=O)=O)cc6)n5n4)cc3)CC2)C(C)(C)C)cc1]

JA-122 was synthesized following the standard procedure for preparing JA-010 (6.1 mg, 60% yield). MS (ESI) m/z: 1032.5 [M+H]⁺.

### Example 211. (2S,4R)-1-((S)-3,3-Dimethyl-2-(7-(2-(4-(4-((5-(4-(methylsulfonyl)phenyl)-[1,2,4]triazolo[1,5-a]pyridin-2-yl)amino)phenyl)piperazin-1-yl)acetamido)heptanamido)butanoyl)-4-hydroxy-N-(4-(4-methylthiazol-5-yl)benzyl)pyrrolidine-2-carboxamide (JA-123)

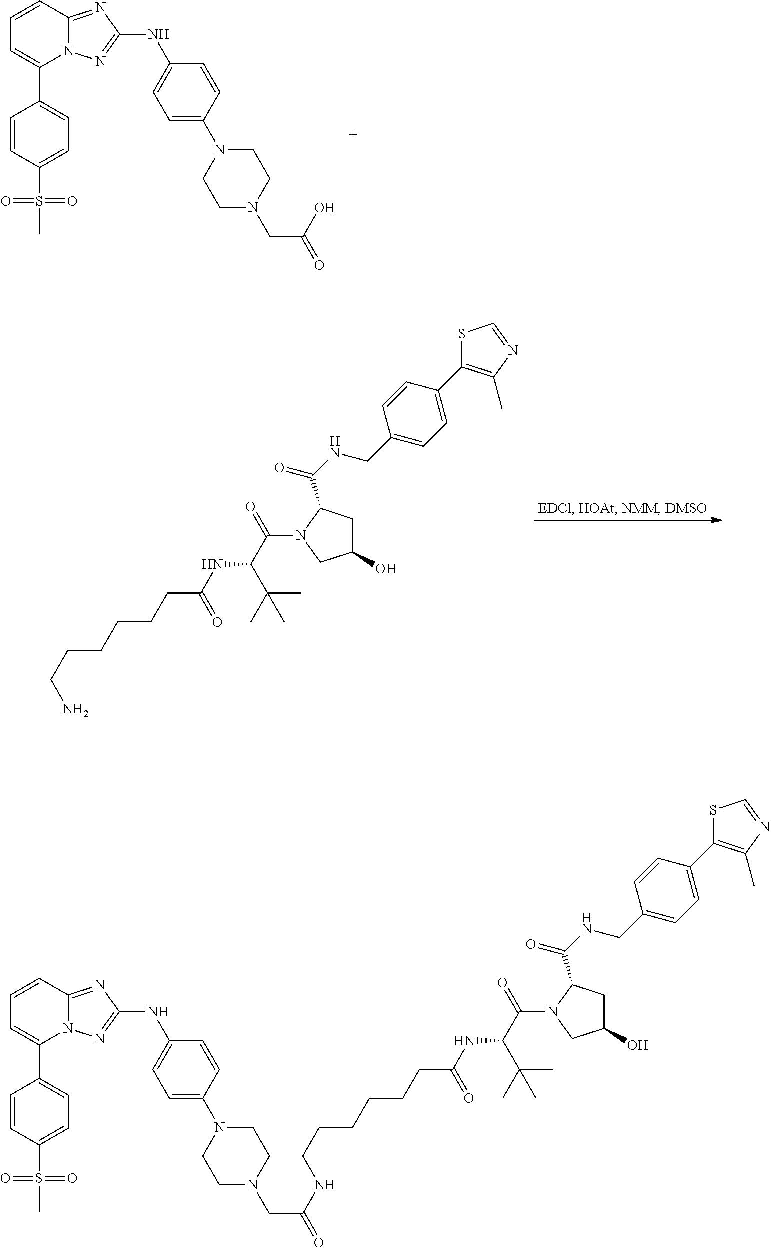 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCCCN)C(C)(C)C)cc1 ; Cc1ncsc1-c1ccc(CNC(=O)[C@@H]2C[C@@H](O)CN2C(=O)[C@@H](NC(=O)CCCCCCNC(=O)CN2CCN(c3ccc(Nc4nc5cccc(-c6ccc(S(C)(=O)=O)cc6)n5n4)cc3)CC2)C(C)(C)C)cc1]

JA-123 was synthesized following the standard procedure for preparing JA-010 (5.6 mg, 50% yield). MS (ESI) m/z: 1046.5 [M+H]⁺.

### Example 212. (2S,4R)-1-((S)-3,3-Dimethyl-2-(8-(2-(4-(4-((5-(4-(methylsulfonyl)phenyl)-[1,2,4]triazolo[1,5-a]pyridin-2-yl)amino)phenyl)piperazin-1-yl)acetamido)octanamido)butanoyl)-4-hydroxy-N-(4-(4-methylthiazol-5-yl)benzyl)pyrrolidine-2-carboxamide (JA-124)

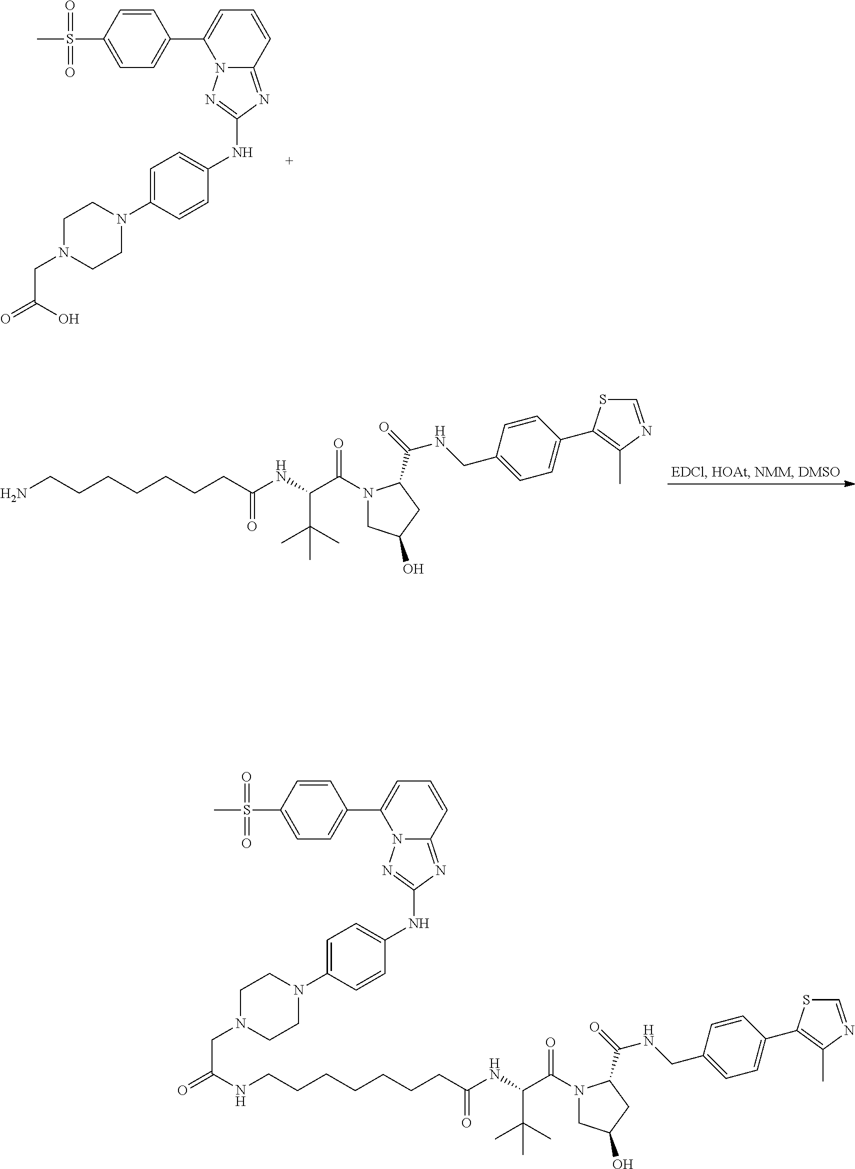 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCCCCN)C(C)(C)C)cc1 ; Cc1ncsc1-c1ccc(CNC(=O)[C@@H]2C[C@@H](O)CN2C(=O)[C@@H](NC(=O)CCCCCCCNC(=O)CN2CCN(c3ccc(Nc4nc5cccc(-c6ccc(S(C)(=O)=O)cc6)n5n4)cc3)CC2)C(C)(C)C)cc1]

JA-124 was synthesized following the standard procedure for preparing JA-110 (5.4 mg, 55% yield). MS (ESI) m/z: 1060.5 [M+H]⁺.

### Example 213. (2S,4R)-1-((S)-3,3-Dimethyl-2-(4-(2-(4-(4-((5-(4-(methylsulfonyl)phenyl)-[1,2,4]triazolo[1,5-a]pyridin-2-yl)amino)phenyl)piperazin-1-yl)acetamido)butanamido)butanoyl)-4-hydroxy-N-(4-(4-methylthiazol-5-yl)benzyl)pyrrolidine-2-carboxamide (JA-125)

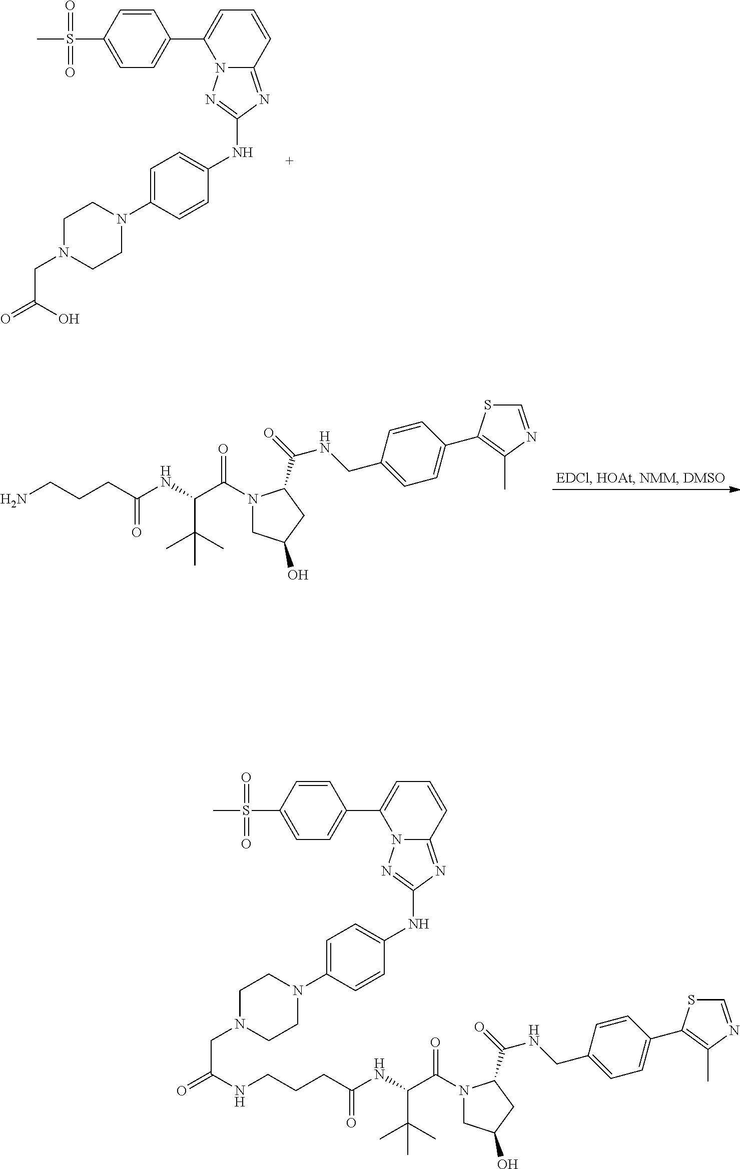 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCN)C(C)(C)C)cc1 ; Cc1ncsc1-c1ccc(CNC(=O)[C@@H]2C[C@@H](O)CN2C(=O)[C@@H](NC(=O)CCCNC(=O)CN2CCN(c3ccc(Nc4nc5cccc(-c6ccc(S(C)(=O)=O)cc6)n5n4)cc3)CC2)C(C)(C)C)cc1]

JA-125 was synthesized following the standard procedure for preparing JA-010 (5.3 mg, 52% yield). MS (ESI) m/z: 1004.4 [M+H]⁺.

### Example 214. (2S,4R)-1-((S)-3,3-Dimethyl-2-(2-(2-(4-(4-((5-(4-(methylsulfonyl)phenyl)-[1,2,4]triazolo[1,5-a]pyridin-2-yl)amino)phenyl)piperazin-1-yl)acetamido)acetamido)butanoyl)-4-hydroxy-N-(4-(4-methylthiazol-5-yl)benzyl)pyrrolidine-2-carboxamide (JA-126)

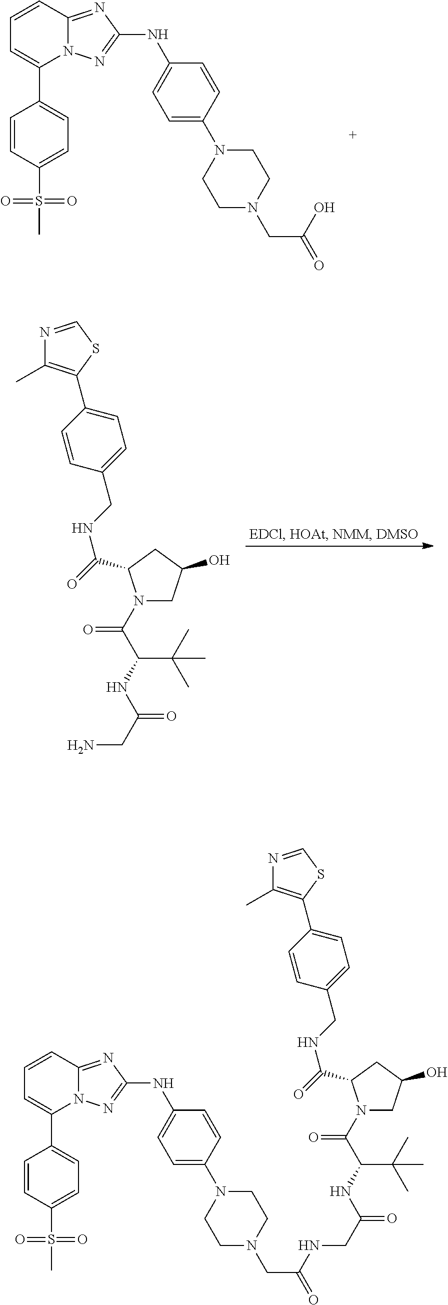 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CN)C(C)(C)C)cc1 ; Cc1ncsc1-c1ccc(CNC(=O)[C@@H]2C[C@@H](O)CN2C(=O)[C@@H](NC(=O)CNC(=O)CN2CCN(c3ccc(Nc4nc5cccc(-c6ccc(S(C)(=O)=O)cc6)n5n4)cc3)CC2)C(C)(C)C)cc1]

JA-126 was synthesized following the standard procedure for preparing JA-010 (5.7 mg, 55% yield). MS (ESI) m/z: 976.4 [M+H]⁺.

### Example 215. N-(2-(2-(2-(2-((2-(2,6-Dioxopiperidin-3-yl)-1,3-dioxoisoindolin-4-yl)amino)ethoxy)ethoxy)ethoxy)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-127)

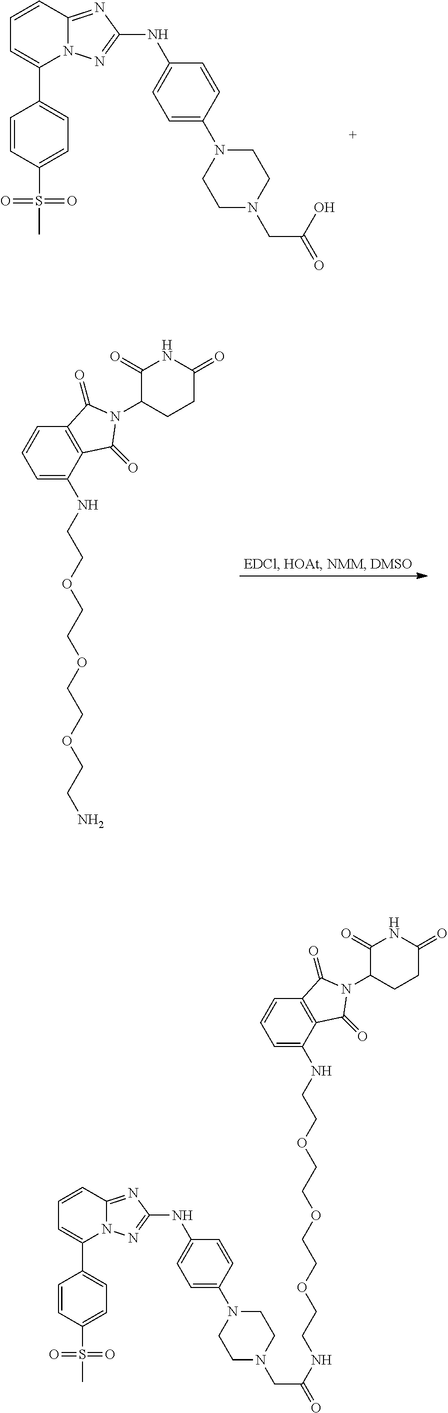 [SMILES: CS(=O)(=O)c1ccc(-c2cccc3nc(Nc4ccc(N5CCN(CC(=O)O)CC5)cc4)nn23)cc1 ; NCCOCCOCCOCCNc1cccc2c1C(=O)N(C1CCC(=O)NC1=O)C2=O ; CS(=O)(=O)c1ccc(-c2cccc3nc(Nc4ccc(N5CCN(CC(=O)NCCOCCOCCOCCNc6cccc7c6C(=O)N(C6CCC(=O)NC6=O)C7=O)CC5)cc4)nn23)cc1]

JA-127 was synthesized following the standard procedure for preparing JA-010 (7.3 mg, 65% yield). MS (ESI) m/z: 937.4 [M+H]⁺.

### Example 216. (2S,4R)-1-((S)-3,3-Dimethyl-2-(11-(2-(4-(4-((5-(4-(methylsulfonyl)phenyl)-[1,2,4]triazolo[1,5-a]pyridin-2-yl)amino)phenyl)piperazin-1-yl)acetamido)undecanamido)butanoyl)-4-hydroxy-N-(4-(4-methylthiazol-5-yl)benzyl)pyrrolidine-2-carboxamide (JA-128)

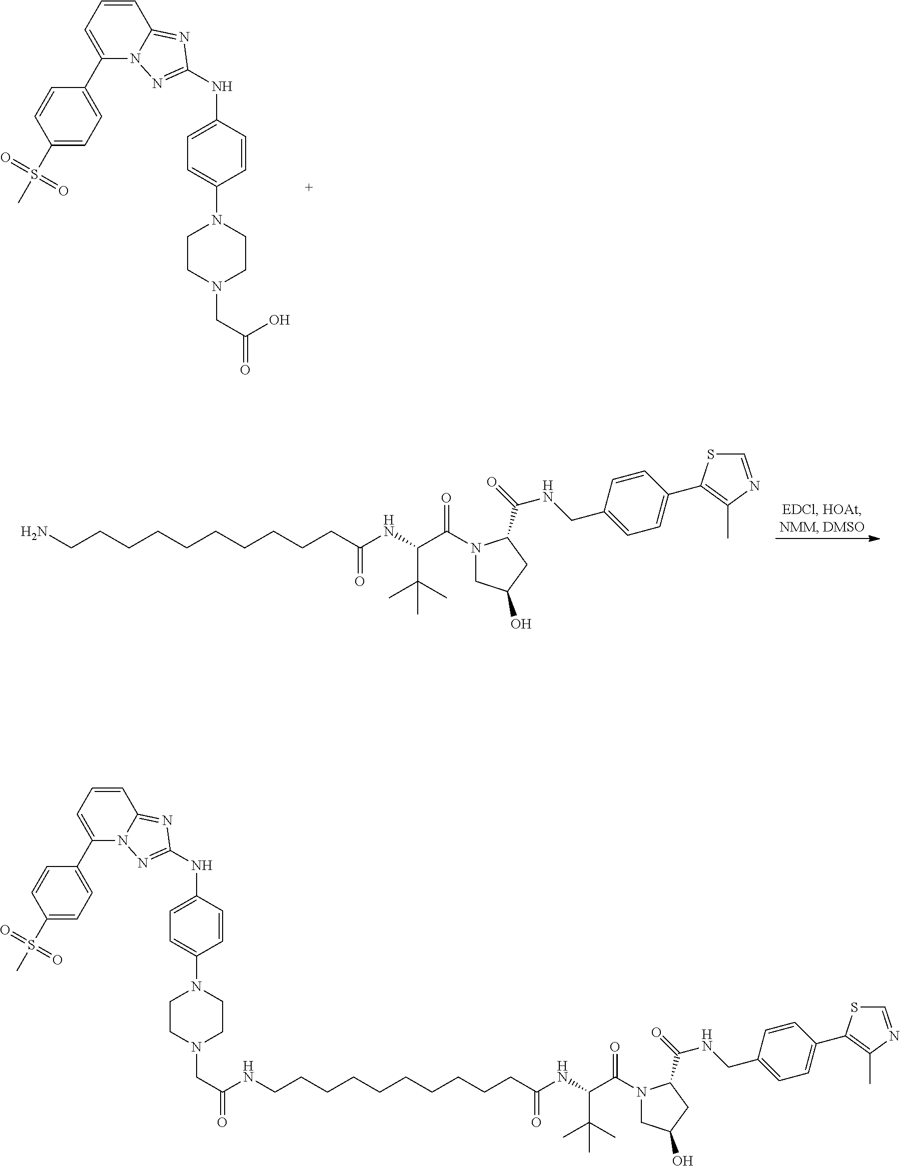 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCCCCCCCN)C(C)(C)C)cc1 ; Cc1ncsc1-c1ccc(CNC(=O)[C@@H]2C[C@@H](O)CN2C(=O)[C@@H](NC(=O)CCCCCCCCCCNC(=O)CN2CCN(c3ccc(Nc4nc5cccc(-c6ccc(S(C)(=O)=O)cc6)n5n4)cc3)CC2)C(C)(C)C)cc1]

JA-128 was synthesized following the standard procedure for preparing JA-010 (5.3 mg, 55% yield). MS (ESI) m/z: 1102.5 [M+H]⁺.

### Example 217. N-(17-((2-(2,6-Dioxopiperidin-3-yl)-1,3-dioxoisoindolin-4-yl)amino)-3,6,9,12,15-pentaoxaheptadecyl)-2-(4-(4-((5-(4-(methylsulfonyl)phenyl)-[1,2,4]triazolo[1,5-a]pyridin-2-yl)amino)phenyl)piperazin-1-yl)acetamide (JA-129)

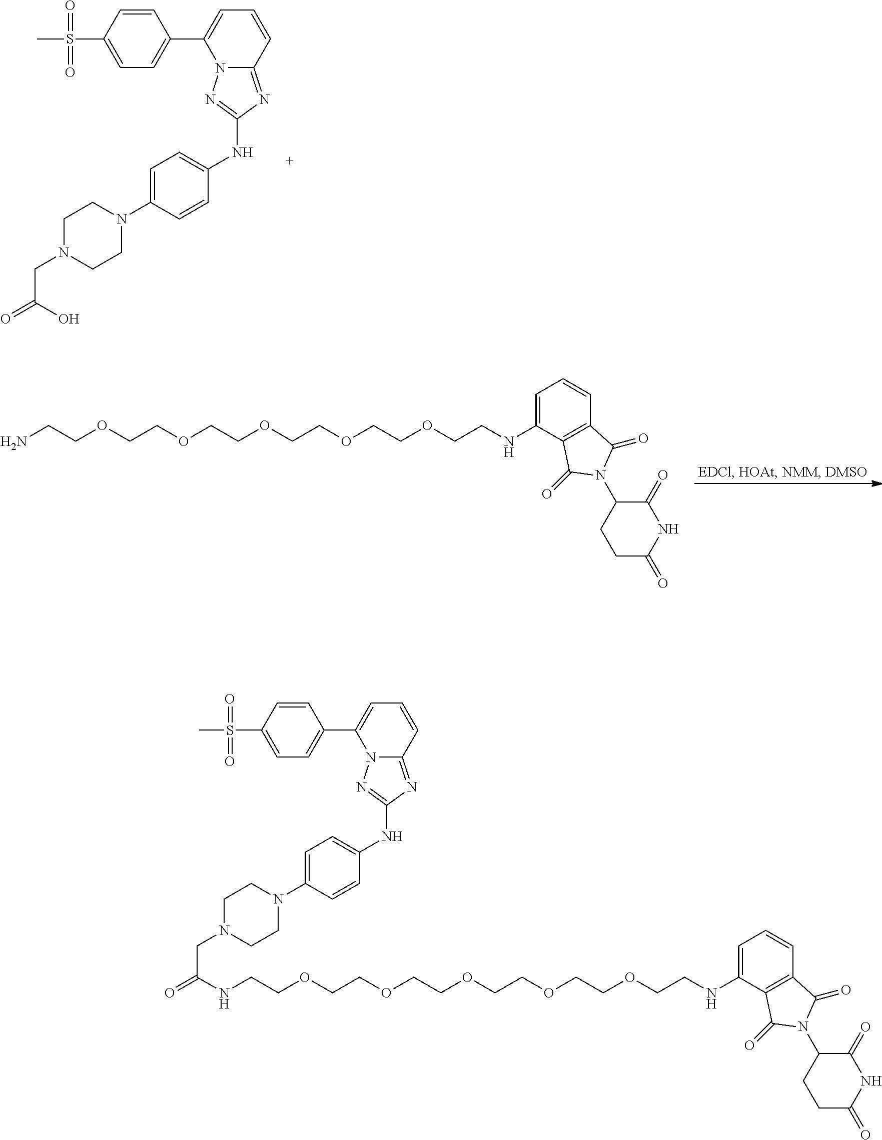 [SMILES: CS(=O)(=O)c1ccc(-c2cccc3nc(Nc4ccc(N5CCN(CC(=O)O)CC5)cc4)nn23)cc1 ; NCCOCCOCCOCCOCCOCCNc1cccc2c1C(=O)N(C1CCC(=O)NC1=O)C2=O ; CS(=O)(=O)c1ccc(-c2cccc3nc(Nc4ccc(N5CCN(CC(=O)NCCOCCOCCOCCOCCOCCNc6cccc7c6C(=O)N(C6CCC(=O)NC6=O)C7=O)CC5)cc4)nn23)cc1]

JA-129 was synthesized following the standard procedure for preparing JA-010 (6.3 mg, 58% yield). MS (ESI) m/z: 1025.4 [M+H]⁺.

### Example 218. (2S,4R)-1-((S)-3,3-Dimethyl-2-(5-(2-(4-(4-((5-(4-(methylsulfonyl)phenyl)-[1,2,4]triazolo[1,5-a]pyridin-2-yl)amino)phenyl)piperazin-1-yl)acetamido)pentanamido)butanoyl)-4-hydroxy-N-(4-(4-methylthiazol-5-yl)benzyl)pyrrolidine-2-carboxamide (JA-130)

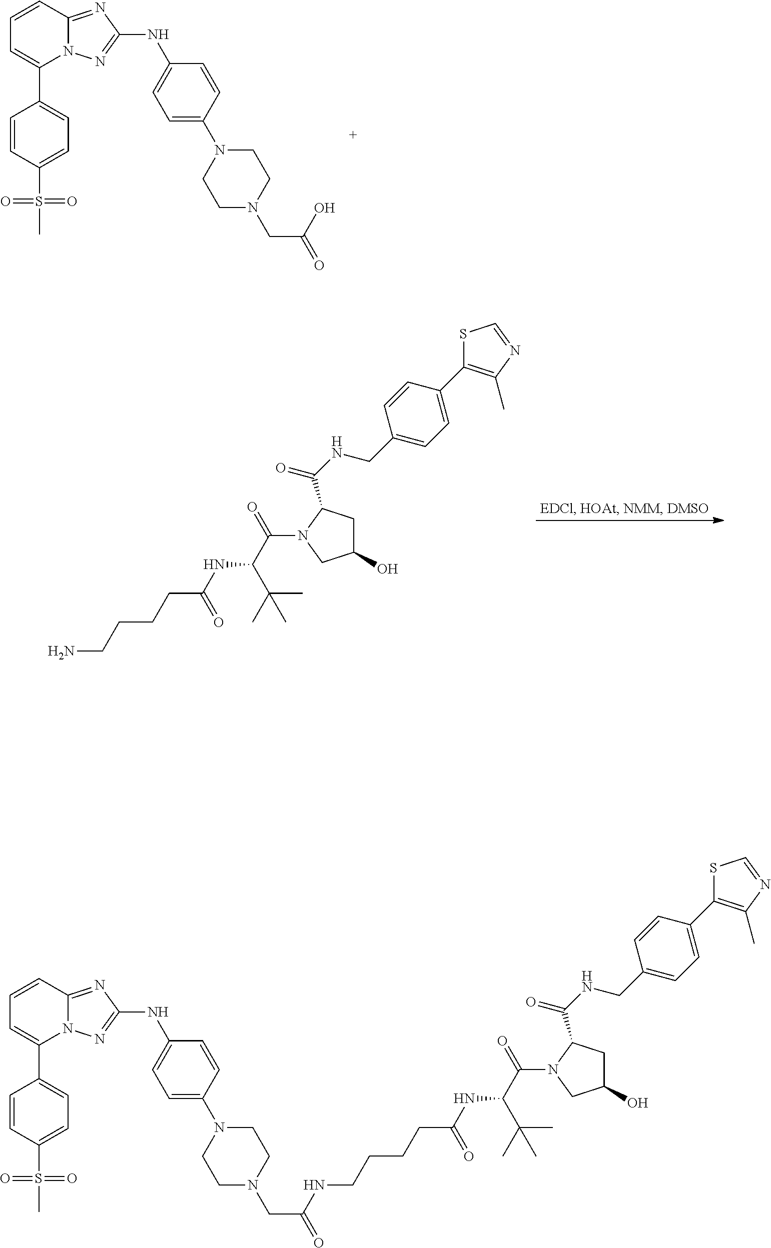 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCCCN)C(C)(C)C)cc1 ; Cc1ncsc1-c1ccc(CNC(=O)[C@@H]2C[C@@H](O)CN2C(=O)[C@@H](NC(=O)CCCCNC(=O)CN2CCN(c3ccc(Nc4nc5cccc(-c6ccc(S(C)(=O)=O)cc6)n5n4)cc3)CC2)C(C)(C)C)cc1]

JA-130 was synthesized following the standard procedure for preparing JA-010 (6.5 mg, 58% yield). MS (ESI) m/z: 1018.4 [M+H]⁺.

### Example 219. (2S,4R)-1-((S)-2-(tert-Butyl)-17-(4-(4-((5-(4-(methylsulfonyl)phenyl)-[1,2,4]triazolo[1,5-a]pyridin-2-yl)amino)phenyl)piperazin-1-yl)-4,16-dioxo-6,9,12-trioxa-3,15-diazaheptadecanoyl)-4-hydroxy-N-(4-(4-methylthiazol-5-yl)benzyl)pyrrolidine-2-carboxamide (JA-131)

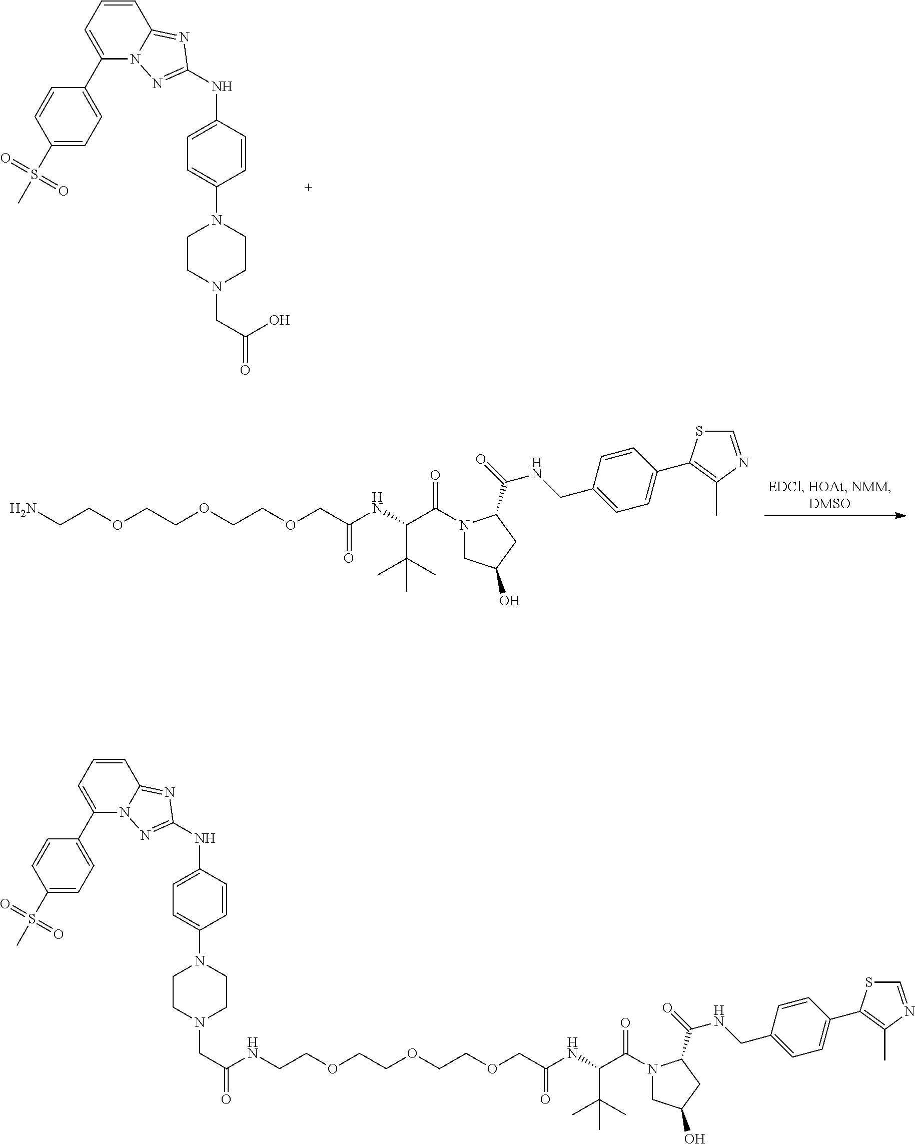 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)COCCOCCOCCN)C(C)(C)C)cc1 ; Cc1ncsc1-c1ccc(CNC(=O)[C@@H]2C[C@@H](O)CN2C(=O)[C@@H](NC(=O)COCCOCCOCCNC(=O)CN2CCN(c3ccc(Nc4nc5cccc(-c6ccc(S(C)(=O)=O)cc6)n5n4)cc3)CC2)C(C)(C)C)cc1]

JA-131 was synthesized following the standard procedure for preparing JA-010 (6.3 mg, 55% yield). MS (ESI) m/z: 1108.5 [M+H]⁺.

### Example 220. (2S,4R)-1-((S)-3,3-Dimethyl-2-(2-(2-(2-(4-(4-((5-(4-(methylsulfonyl)phenyl)-[1,2,4]triazolo[1,5-a]pyridin-2-yl)amino)phenyl)piperazin-1-yl)acetamido)ethoxy)acetamido)butanoyl)-4-hydroxy-N-(4-(4-methylthiazol-5-yl)benzyl)pyrrolidine-2-carboxamide (JA-132)

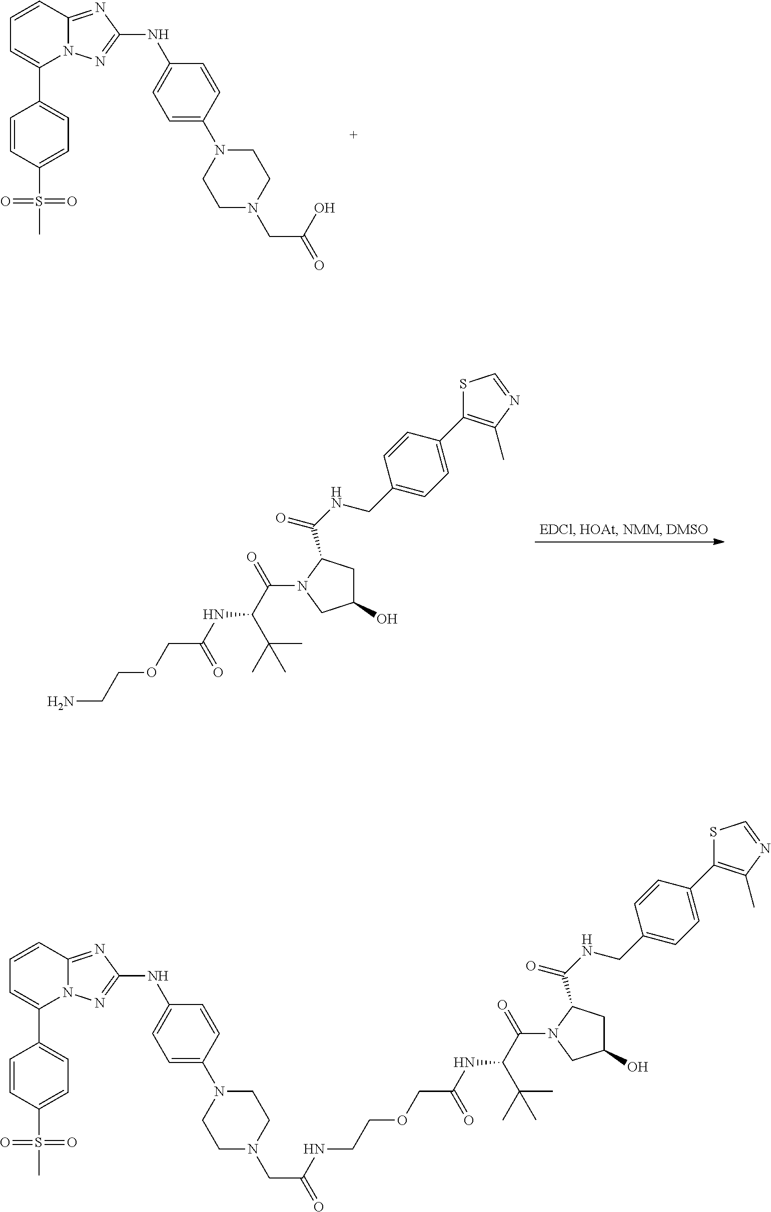 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)COCCN)C(C)(C)C)cc1 ; Cc1ncsc1-c1ccc(CNC(=O)[C@@H]2C[C@@H](O)CN2C(=O)[C@@H](NC(=O)COCCNC(=O)CN2CCN(c3ccc(Nc4nc5cccc(-c6ccc(S(C)(=O)=O)cc6)n5n4)cc3)CC2)C(C)(C)C)cc1]

JA-132 was synthesized following the standard procedure for preparing JA-010 (6.1 mg, 50% yield). MS (ESI) m/z: 1020.4 [M+H]⁺.

### Example 221. (2S,4R)-1-((S)-20-(tert-Butyl)-1-(4-(4-((5-(4-(methylsulfonyl)phenyl)-[1,2,4]triazolo[1,5-a]pyridin-2-yl)amino)phenyl)piperazin-1-yl)-2,18-dioxo-6,9,12,15-tetraoxa-3,19-diazahenicosan-21-oyl)-4-hydroxy-N-(4-(4-methylthiazol-5-yl)benzyl)pyrrolidine-2-carboxamide (JA-133)

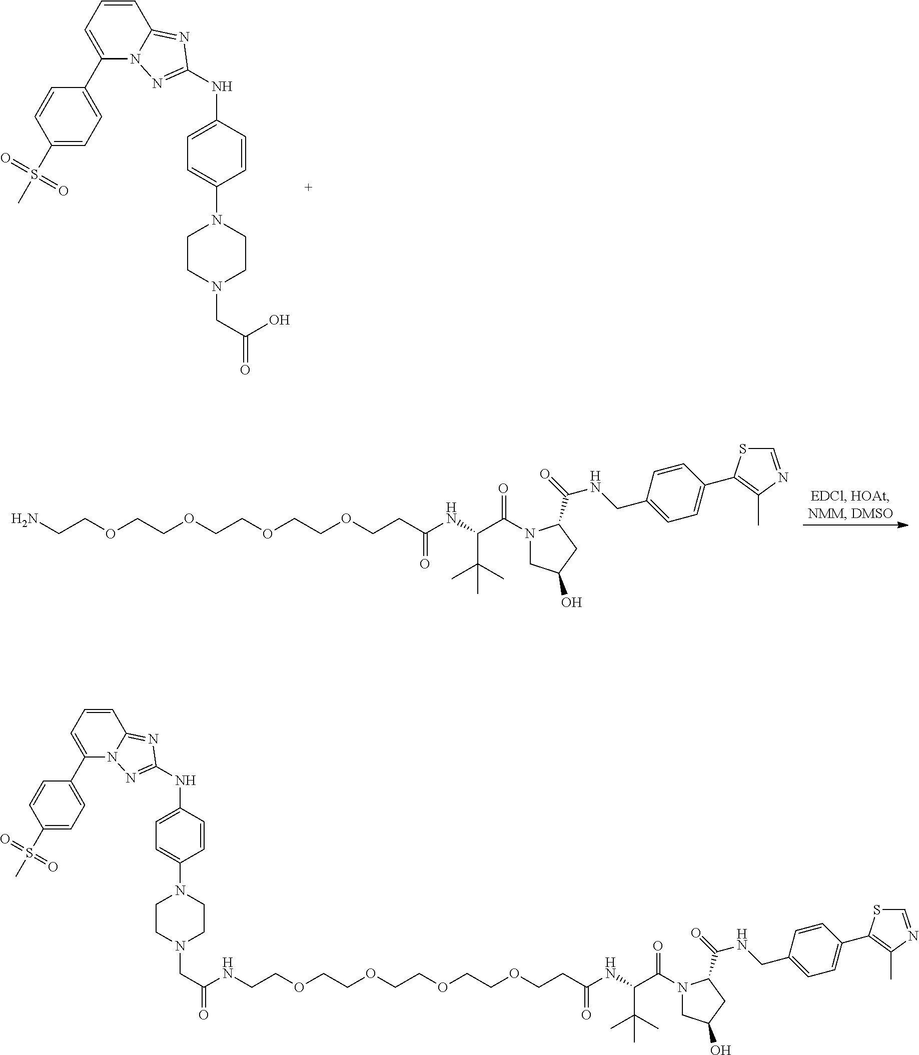 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCOCCOCCNC(=O)CN2CCN(c3ccc(Nc4nc5cccc(-c6ccc(S(C)(=O)=O)cc6)n5n4)cc3)CC2)C(C)(C)C)cc1]

JA-133 was synthesized following the standard procedure for preparing JA-010 (6.1 mg, 52% yield). MS (ESI) m/z: 1166.5 [M+H]⁺.

### Example 222. (2S,4R)-1-((S)-2-(tert-Butyl)-14-(4-(4-((5-(4-(methylsulfonyl)phenyl)-[1,2,4]triazolo[1,5-a]pyridin-2-yl)amino)phenyl)piperazin-1-yl)-4,13-dioxo-6,9-dioxa-3,12-diazatetradecanoyl)-4-hydroxy-N-(4-(4-methylthiazol-5-yl)benzyl)pyrrolidine-2-carboxamide (JA-134)

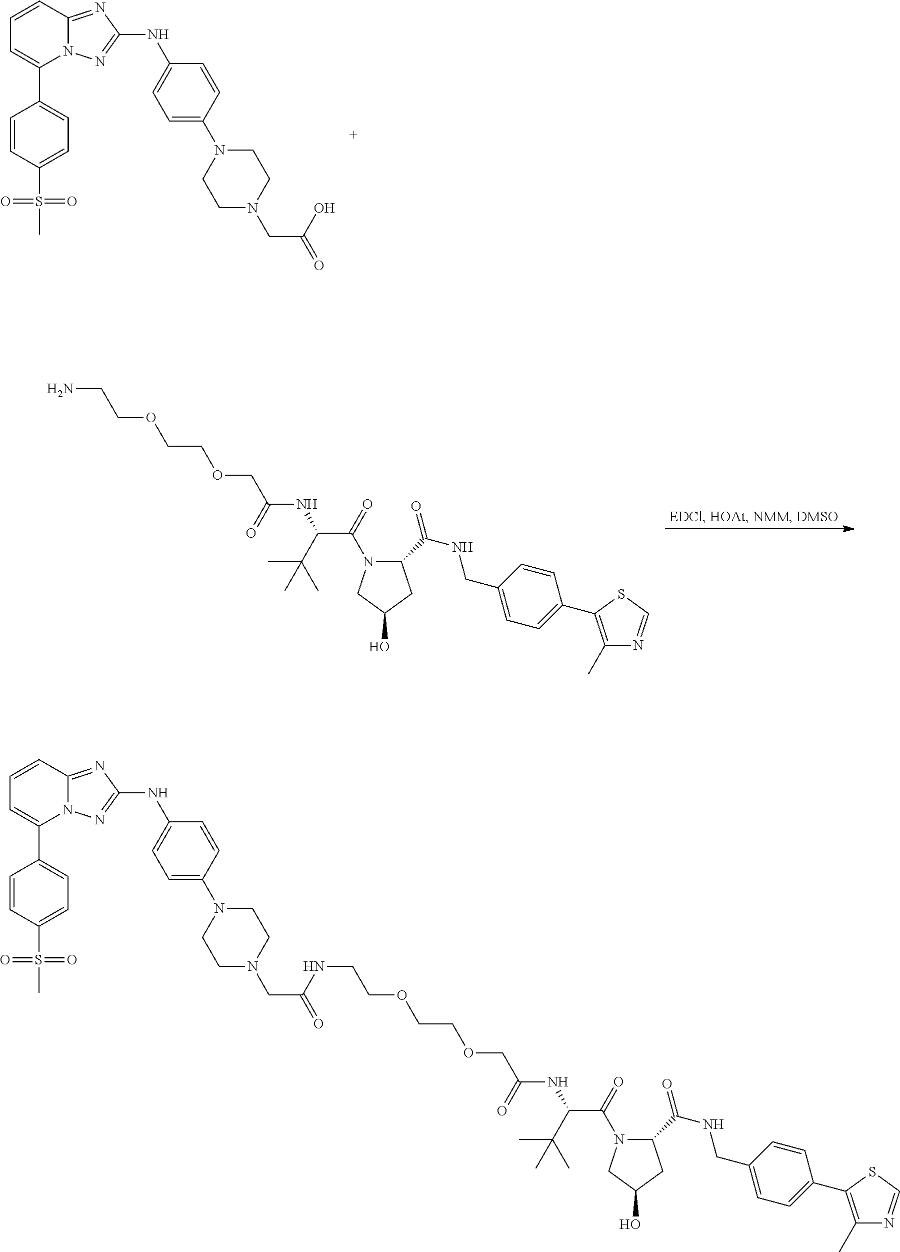 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)COCCOCCN)C(C)(C)C)cc1 ; Cc1ncsc1-c1ccc(CNC(=O)[C@@H]2C[C@@H](O)CN2C(=O)[C@@H](NC(=O)COCCOCCNC(=O)CN2CCN(c3ccc(Nc4nc5cccc(-c6ccc(S(C)(=O)=O)cc6)n5n4)cc3)CC2)C(C)(C)C)cc1]

JA-134 was synthesized following the standard procedure for preparing JA-010 (5.7 mg, 55% yield). MS (ESI) m/z: 1064.5 [M+H]⁺.

### Example 223. (2S,4R)-1-((S)-14-(tert-Butyl)-1-(4-(4-((5-(4-(methylsulfonyl)phenyl)-[1,2,4]triazolo[1,5-a]pyridin-2-yl)amino)phenyl)piperazin-1-yl)-2,12-dioxo-6,9-dioxa-3,13-diazapentadecan-15-oyl)-4-hydroxy-N-(4-(4-methylthiazol-5-yl)benzyl)pyrrolidine-2-carboxamide (JA-135)

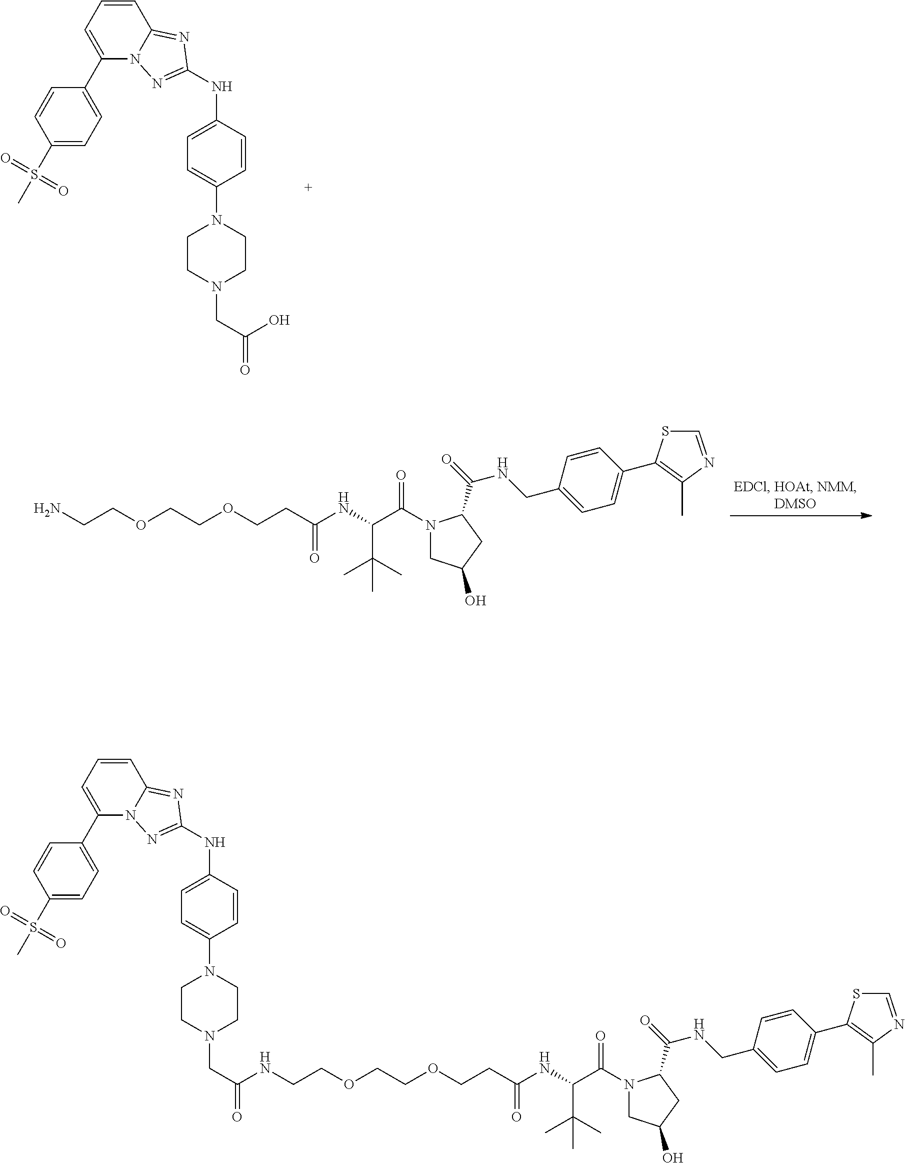 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCNC(=O)CN2CCN(c3ccc(Nc4nc5cccc(-c6ccc(S(C)(=O)=O)cc6)n5n4)cc3)CC2)C(C)(C)C)cc1]

JA-135 was synthesized following the standard procedure for preparing JA-010 (6.7 mg, 60% yield). MS (ESI) m/z: 1078.5 [M+H]⁺.

### Example 224. (2S,4R)-1-((S)-3,3-Dimethyl-2-(3-(2-(2-(4-(4-((5-(4-(methylsulfonyl)phenyl)-[1,2,4]triazolo[1,5-a]pyridin-2-yl)amino)phenyl)piperazin-1-yl)acetamido)ethoxy)propanamido)butanoyl)-4-hydroxy-N-(4-(4-methylthiazol-5-yl)benzyl)pyrrolidine-2-carboxamide (JA-136)

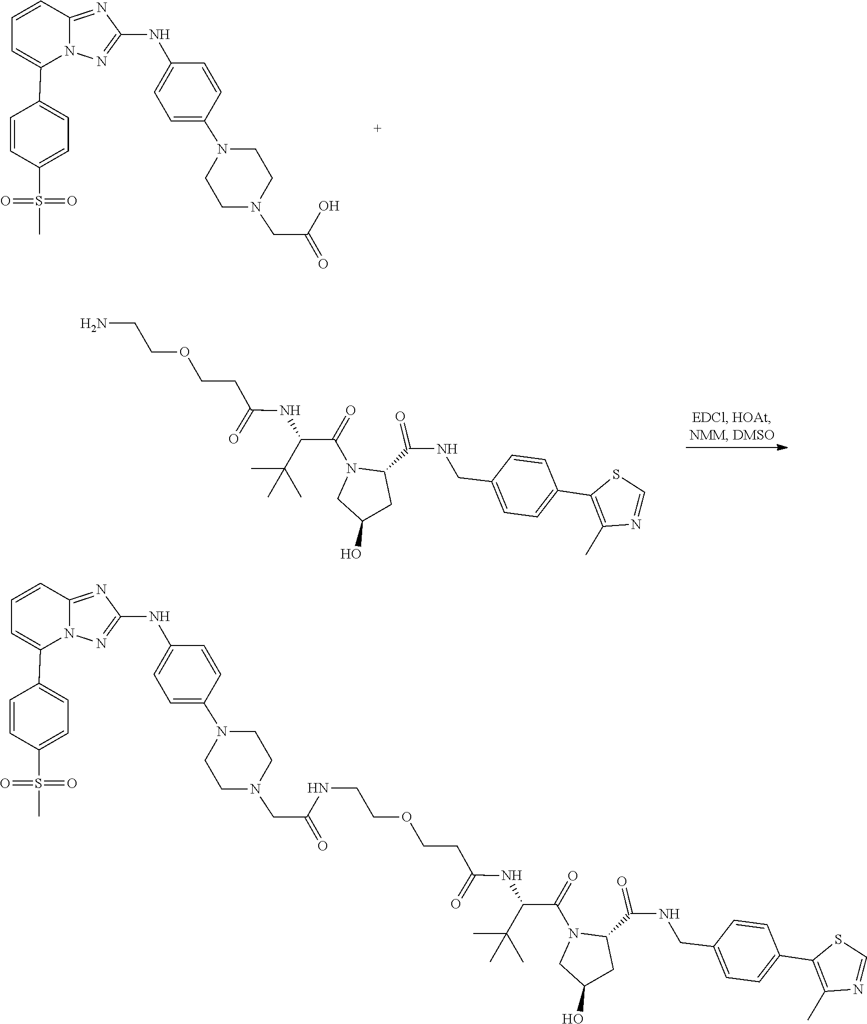 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CN2C(=O)[C@@H](NC(=O)CCOCCN)C(C)(C)C)cc1 ; Cc1ncsc1-c1ccc(CNC(=O)[C@@H]2C[C@@H](O)CN2C(=O)[C@@H](NC(=O)CCOCCNC(=O)CN2CCN(c3ccc(Nc4nc5cccc(-c6ccc(S(C)(=O)=O)cc6)n5n4)cc3)CC2)C(C)(C)C)cc1]

JA-136 was synthesized following the standard procedure for preparing JA-010 (6.0 mg, 60% yield). MS (ESI) m/z: 1034.4 [M+H]⁺.

### Example 225. (2S,4R)-1-((S)-17-(tert-Butyl)-1-(4-(4-((5-(4-(methylsulfonyl)phenyl)-[1,2,4]triazolo[1,5-a]pyridin-2-yl)amino)phenyl)piperazin-1-yl)-2,15-dioxo-6,9,12-trioxa-3,16-diazaoctadecan-18-oyl)-4-hydroxy-N-(4-(4-methylthiazol-5-yl)benzyl)pyrrolidine-2-carboxamide (JA-137)

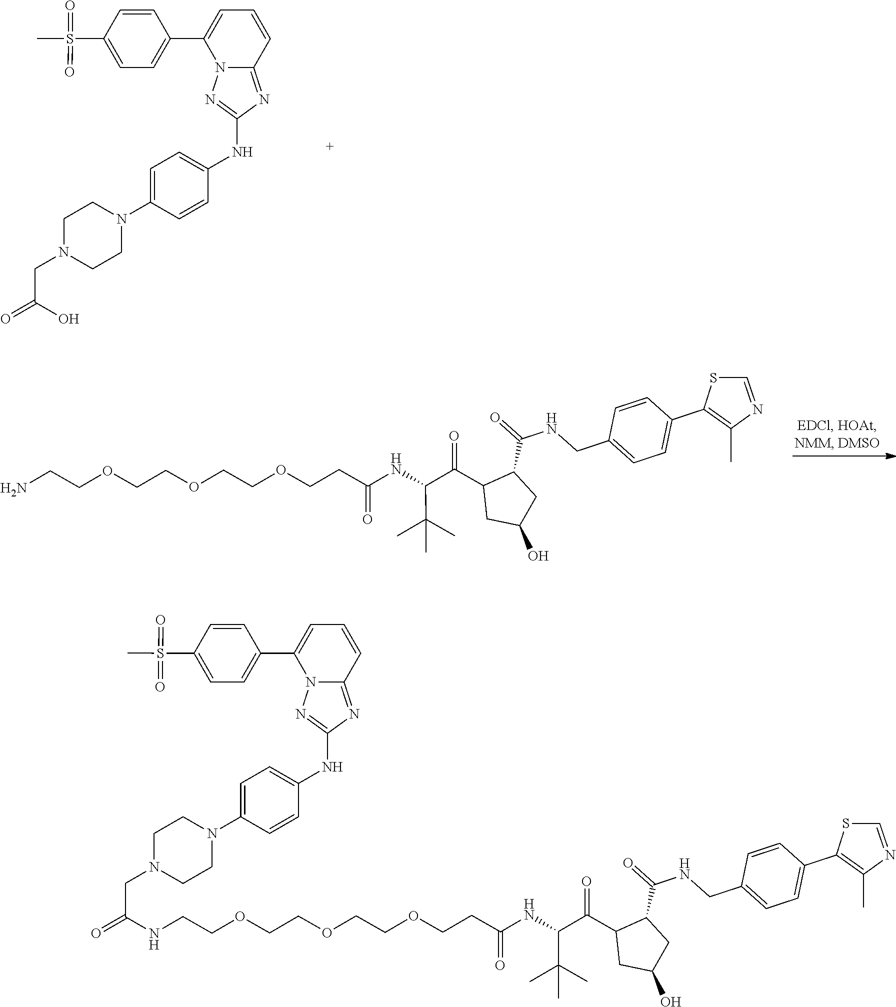 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)[C@@H]2C[C@@H](O)CC2C(=O)[C@@H](NC(=O)CCOCCOCCOCCN)C(C)(C)C)cc1 ; Cc1ncsc1-c1ccc(CNC(=O)[C@@H]2C[C@@H](O)CC2C(=O)[C@@H](NC(=O)CCOCCOCCOCCNC(=O)CN2CCN(c3ccc(Nc4nc5cccc(-c6ccc(S(C)(=O)=O)cc6)n5n4)cc3)CC2)C(C)(C)C)cc1]

JA-137 was synthesized following the standard procedure for preparing JA-010 (5.0 mg, 61% yield). MS (ESI) m/z: 1122.5 [M+H]⁺.

### Example 226. (2S,4R)-1-((S)-23-(tert-Butyl)-1-(4-(4-((5-(4-(methylsulfonyl)phenyl)-[1,2,4]triazolo[1,5-a]pyridin-2-yl)amino)phenyl)piperazin-1-yl)-2,21-dioxo-6,9,12,15,18-pentaoxa-3,22-diazatetracosan-24-oyl)-4-hydroxy-N-(4-(4-methylthiazol-5-yl)benzyl)pyrrolidine-2-carboxamide (JA-138)

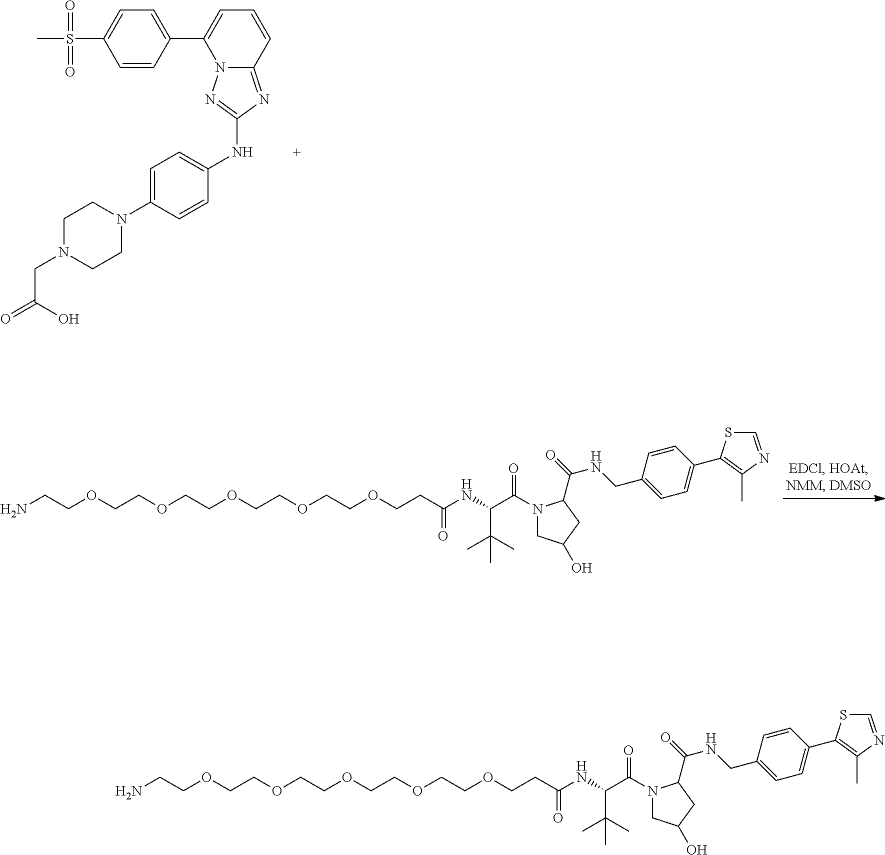 [SMILES: CS(=O)(=O)c1ccc(-c2cccc3nc(Nc4ccc(N5CCN(CC(=O)O)CC5)cc4)nn23)cc1 ; Cc1ncsc1-c1ccc(CNC(=O)C2CC(O)CN2C(=O)[C@@H](NC(=O)CCOCCOCCOCCOCCOCCN)C(C)(C)C)cc1 ; Cc1ncsc1-c1ccc(CNC(=O)C2CC(O)CN2C(=O)[C@@H](NC(=O)CCOCCOCCOCCOCCOCCN)C(C)(C)C)cc1]

JA-138 was synthesized following the standard procedure for preparing JA-010 (6.5 mg, 62% yield). MS (ESI) m/z: 1210.5 [M+H]⁺.

### Example 227. 2-[4-[4-[8-[3,5-Difluoro-4-(morpholinomethyl)phenyl]quinoxalin-2-yl]pyrazol-1-yl]-1-piperidyl]-N-[2-[2-[[2-(2,6-dioxo-3-piperidyl)-1,3-dioxo-isoindolin-4-yl]amino]ethoxy]ethyl]acetamide (JA-139)

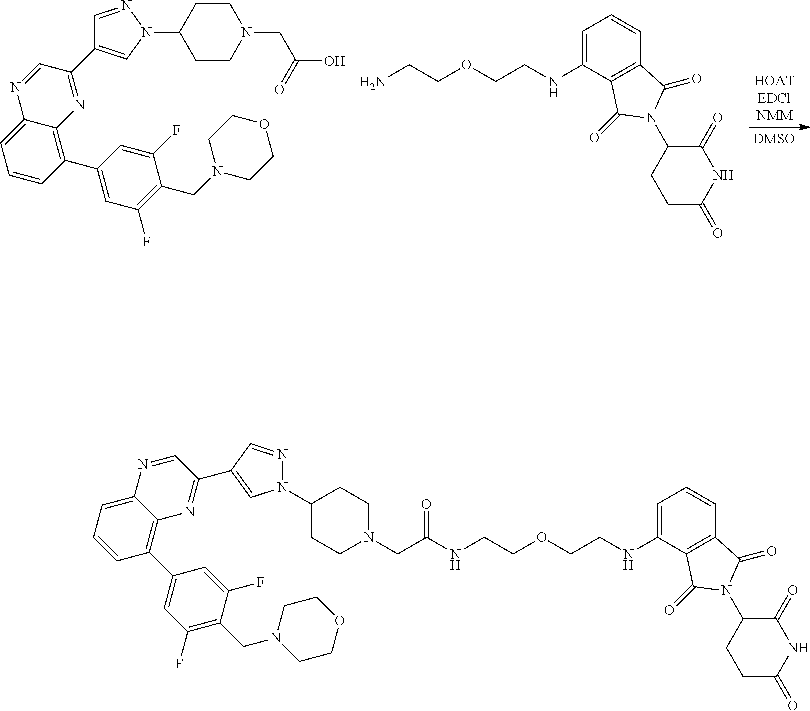 [SMILES: O=C(O)CN1CCC(n2cc(-c3cnc4cccc(-c5cc(F)c(CN6CCOCC6)c(F)c5)c4n3)cn2)CC1 ; NCCOCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCNc1cccc2c1C(=O)N(C1CCC(=O)NC1=O)C2=O]

JA-139 was synthesized following the standard procedure for preparing JA-001 (7.9 mg, 47.4% yield). MS (ESI) m/z: 891.4 [M+H]⁺.

### Example 228. 2-[4-[4-[8-[3,5-Difluoro-4-(morpholinomethyl)phenyl]quinoxalin-2-yl]pyrazol-1-yl]-1-piperidyl]-N-[2-[2-[2-[[2-(2,6-dioxo-3-piperidyl)-1,3-dioxo-isoindolin-4-yl]amino]ethoxy]ethoxy]ethyl]acetamide (JA-140)

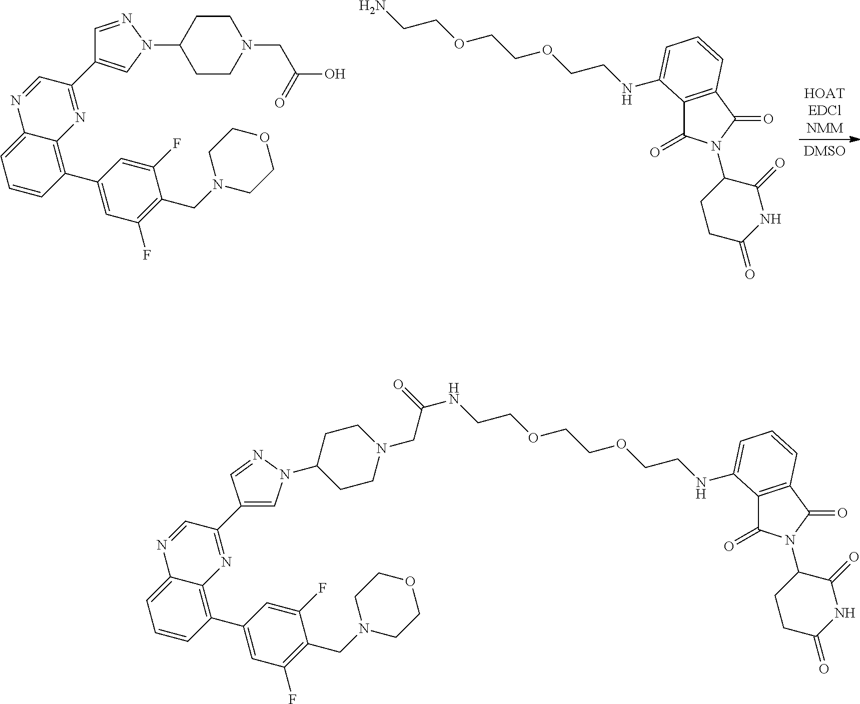 [SMILES: O=C(O)CN1CCC(n2cc(-c3cnc4cccc(-c5cc(F)c(CN6CCOCC6)c(F)c5)c4n3)cn2)CC1 ; NCCOCCOCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCNc1cccc2c1C(=O)N(C1CCC(=O)NC1=O)C2=O]

JA-140 was synthesized following the standard procedure for preparing JA-001 (8.6 mg, 49.1% yield). MS (ESI) m/z: 935.4 [M+H]⁺.

### Example 229. 2-[4-[4-[8-[3,5-Difluoro-4-(morpholinomethyl)phenyl]quinoxalin-2-yl]pyrazol-1-yl]-1-piperidyl]-N-[2-[2-[2-[2-[[2-(2,6-dioxo-3-piperidyl)-1,3-dioxo-isoindolin-4-yl]amino]ethoxy]ethoxy]ethoxy]ethyl]acetamide (JA-141)

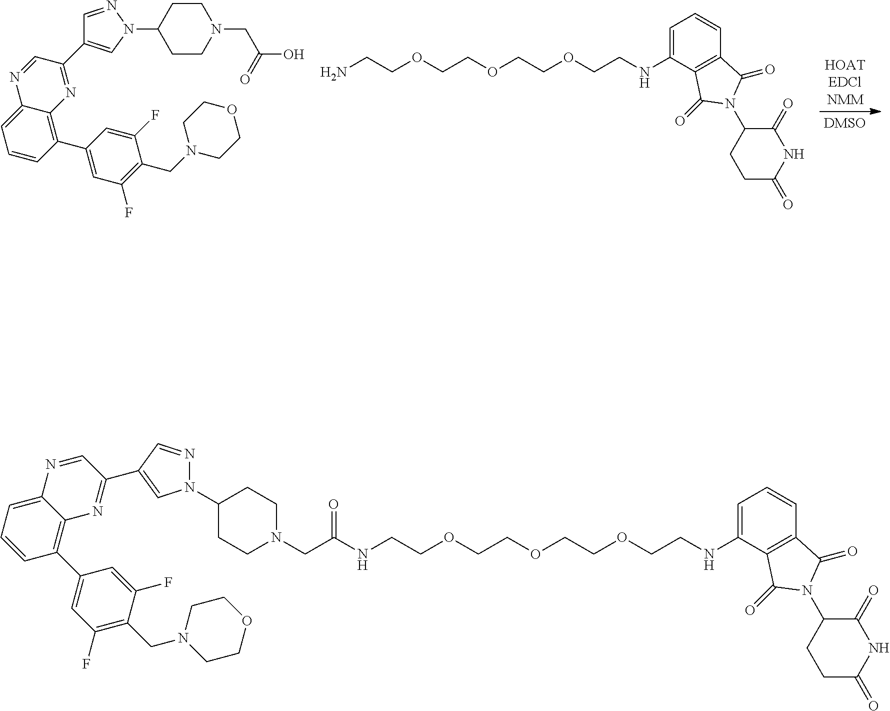 [SMILES: O=C(O)CN1CCC(n2cc(-c3cnc4cccc(-c5cc(F)c(CN6CCOCC6)c(F)c5)c4n3)cn2)CC1 ; NCCOCCOCCOCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCOCCNc1cccc2c1C(=O)N(C1CCC(=O)NC1=O)C2=O]

JA-141 was synthesized following the standard procedure for preparing JA-001 (9.8 mg, 53.5% yield). MS (ESI) m/z: 979.4 [M+H]⁺.

### Example 230. 2-[4-[4-[8-[3,5-Difluoro-4-(morpholinomethyl)phenyl]quinoxalin-2-yl]pyrazol-1-yl]-1-piperidyl]-N-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl]acetamide (JA-142)

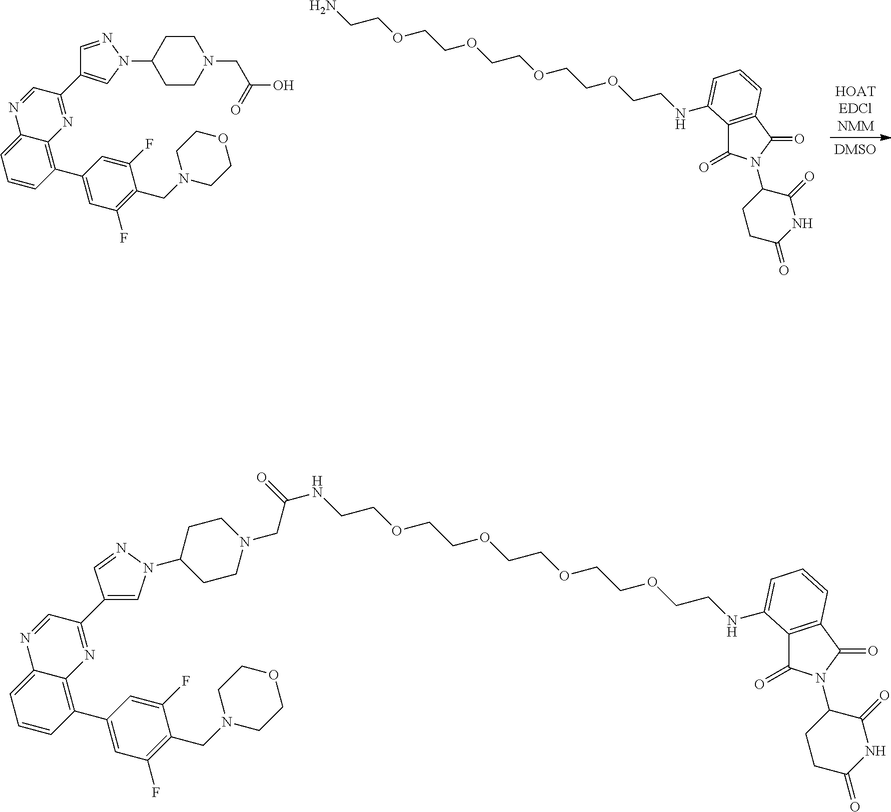 [SMILES: O=C(O)CN1CCC(n2cc(-c3cnc4cccc(-c5cc(F)c(CN6CCOCC6)c(F)c5)c4n3)cn2)CC1 ; NCCOCCOCCOCCOCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCOCCOCCNc1cccc2c1C(=O)N(C1CCC(=O)NC1=O)C2=O]

JA-142 was synthesized following the standard procedure for preparing JA-001 (10.9 mg, 56.9% yield). MS (ESI) m/z: 1023.4 [M+H]⁺.

### Example 231. 2-[4-[4-[8-[3,5-Difluoro-4-(morpholinomethyl)phenyl]quinoxalin-2-yl]pyrazol-1-yl]-1-piperidyl]-N-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethyl]acetamide (JA-143)

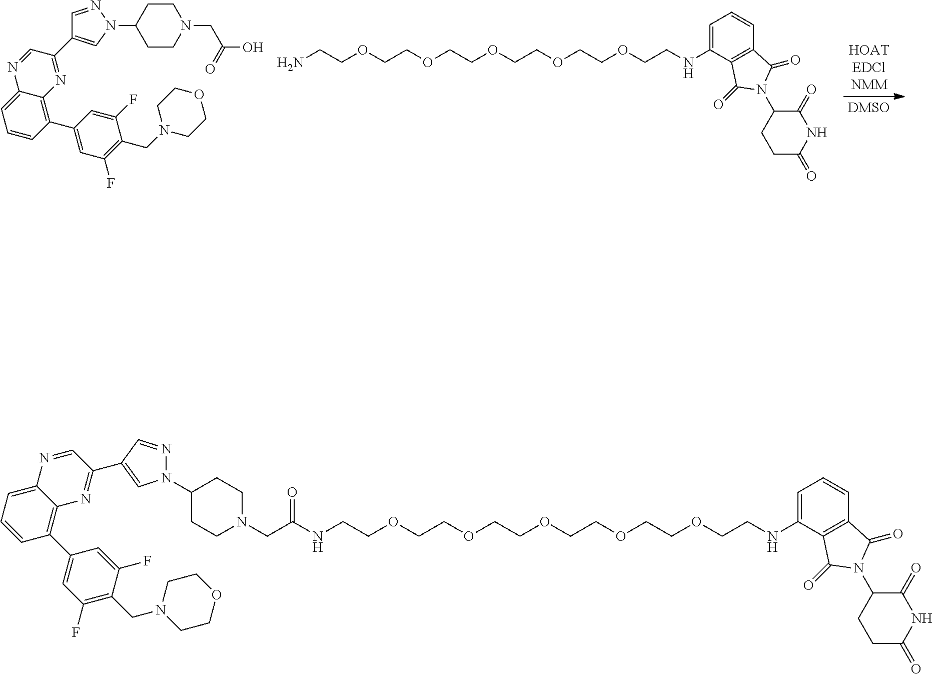 [SMILES: O=C(O)CN1CCC(n2cc(-c3cnc4cccc(-c5cc(F)c(CN6CCOCC6)c(F)c5)c4n3)cn2)CC1 ; NCCOCCOCCOCCOCCOCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCOCCOCCOCCNc1cccc2c1C(=O)N(C1CCC(=O)NC1=O)C2=O]

JA-143 was synthesized following the standard procedure for preparing JA-001 (9.8 mg, 49.1% yield). MS (ESI) m/z: 1067.5 [M+H]⁺.

### Example 232. 2-[4-[4-[8-[3,5-Difluoro-4-(morpholinomethyl)phenyl]quinoxalin-2-yl]pyrazol-1-yl]-1-piperidyl]-N-[2-[[2-(2,6-dioxo-3-piperidyl)-1,3-dioxo-isoindolin-4-yl]amino]ethyl]acetamide (JA-144)

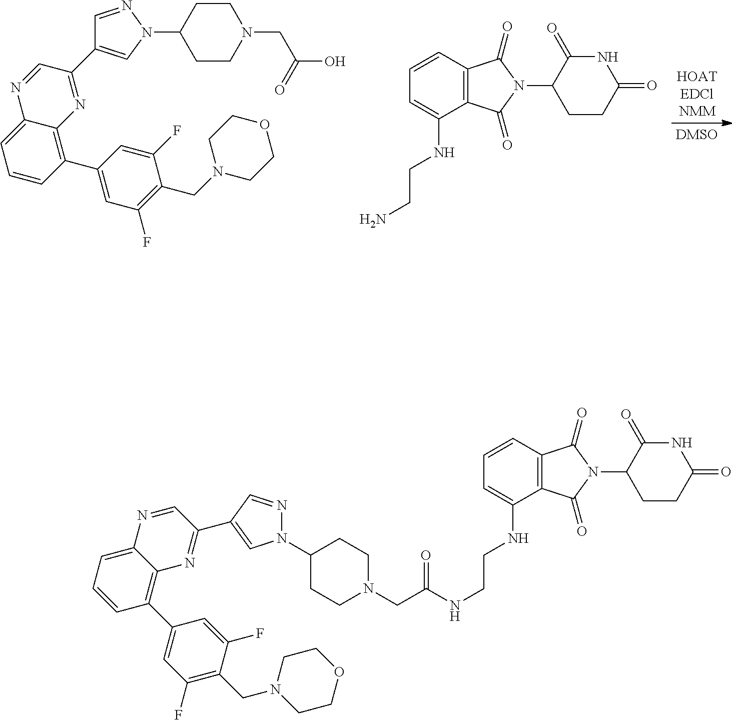 [SMILES: O=C(O)CN1CCC(n2cc(-c3cnc4cccc(-c5cc(F)c(CN6CCOCC6)c(F)c5)c4n3)cn2)CC1 ; NCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCNc1cccc2c1C(=O)N(C1CCC(=O)NC1=O)C2=O]

JA-144 was synthesized following the standard procedure for preparing JA-001 (7.9 mg, 49.8% yield). MS (ESI) m/z: 847.3 [M+H]⁺.

### Example 233. 2-[4-[4-[8-[3,5-Difluoro-4-(morpholinomethyl)phenyl]quinoxalin-2-yl]pyrazol-1-yl]-1-piperidyl]-N-[3-[[2-(2,6-dioxo-3-piperidyl)-1,3-dioxo-isoindolin-4-yl]amino]propyl]acetamide (JA-145)

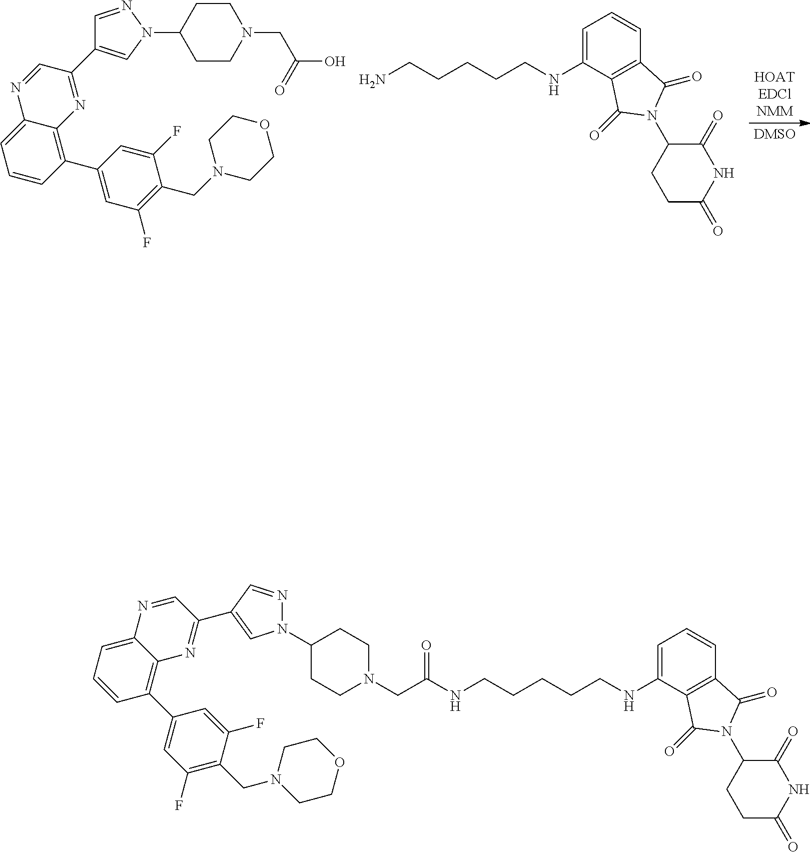 [SMILES: O=C(O)CN1CCC(n2cc(-c3cnc4cccc(-c5cc(F)c(CN6CCOCC6)c(F)c5)c4n3)cn2)CC1 ; NCCCCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCCNc1cccc2c1C(=O)N(C1CCC(=O)NC1=O)C2=O]

JA-145 was synthesized following the standard procedure for preparing JA-001 (8.1 mg, 50.29% yield). MS (ESI) m/z: 889.4 [M+H]⁺.

### Example 234. 2-(4-(4-(8-(3,5-Difluoro-4-(morpholinomethyl)phenyl)quinoxalin-2-yl)-1H-pyrazol-1-yl)piperidin-1-yl)-N-(4-((2-(2,6-dioxopiperidin-3-yl)-1,3-dioxoisoindolin-4-yl)amino)butyl)acetamide (JA-146)

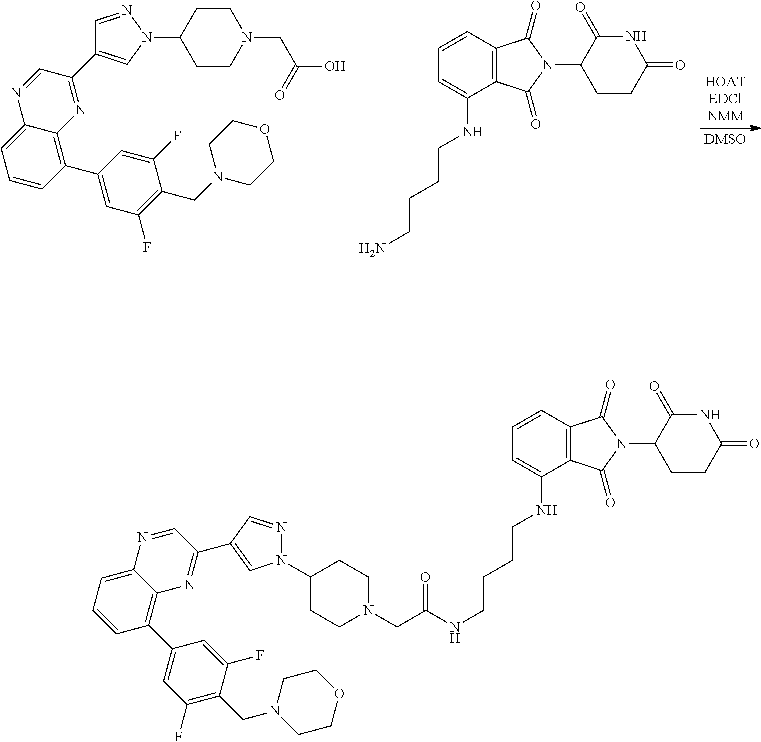 [SMILES: O=C(O)CN1CCC(n2cc(-c3cnc4cccc(-c5cc(F)c(CN6CCOCC6)c(F)c5)c4n3)cn2)CC1 ; NCCCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCNc1cccc2c1C(=O)N(C1CCC(=O)NC1=O)C2=O]

JA-146 was synthesized following the standard procedure for preparing JA-001 (8.0 mg, 48.8% yield). MS (ESI) m/z: 875.4 [M+H]⁺.

### Example 235. 2-(4-(4-(8-(3,5-Difluoro-4-(morpholinomethyl)phenyl)quinoxalin-2-yl)-1H-pyrazol-1-yl)piperidin-1-yl)-N-(6-((2-(2,6-dioxopiperidin-3-yl)-1,3-dioxoisoindolin-4-yl)amino)hexyl)acetamide (JA-147)

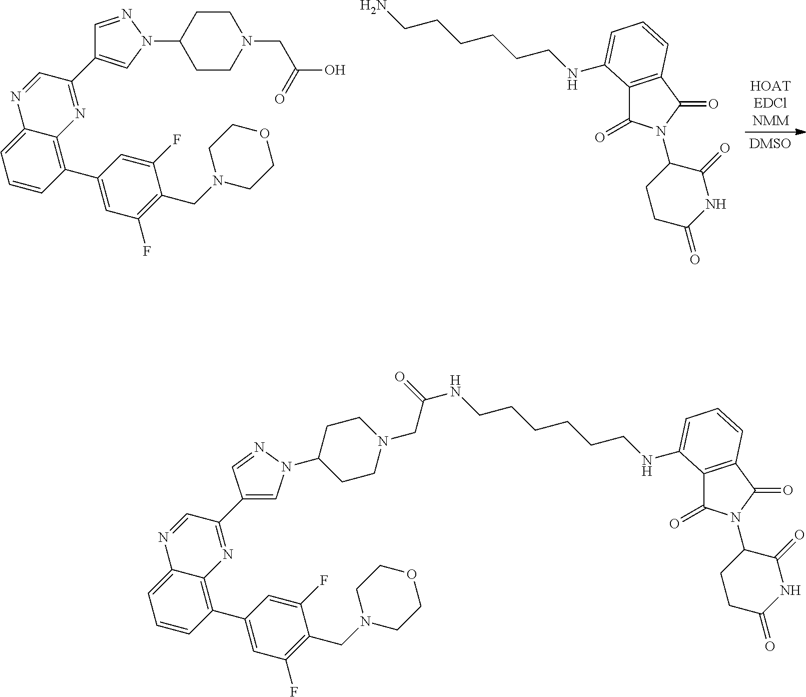 [SMILES: O=C(O)CN1CCC(n2cc(-c3cnc4cccc(-c5cc(F)c(CN6CCOCC6)c(F)c5)c4n3)cn2)CC1 ; NCCCCCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCCCNc1cccc2c1C(=O)N(C1CCC(=O)NC1=O)C2=O]

JA-147 was synthesized following the standard procedure for preparing JA-001 (9.5 mg, 56.2% yield). MS (ESI) m/z: 903.4 [M+H]⁺.

### Example 236

### 2-(4-(4-(8-(3,5-Difluoro-4-(morpholinomethyl)phenyl)quinoxalin-2-yl)-1H-pyrazol-1-yl)piperidin-1-yl)-N-(7-((2-(2,6-dioxopiperidin-3-yl)-1,3-dioxoisoindolin-4-yl)amino)heptyl)acetamide (JA-148)

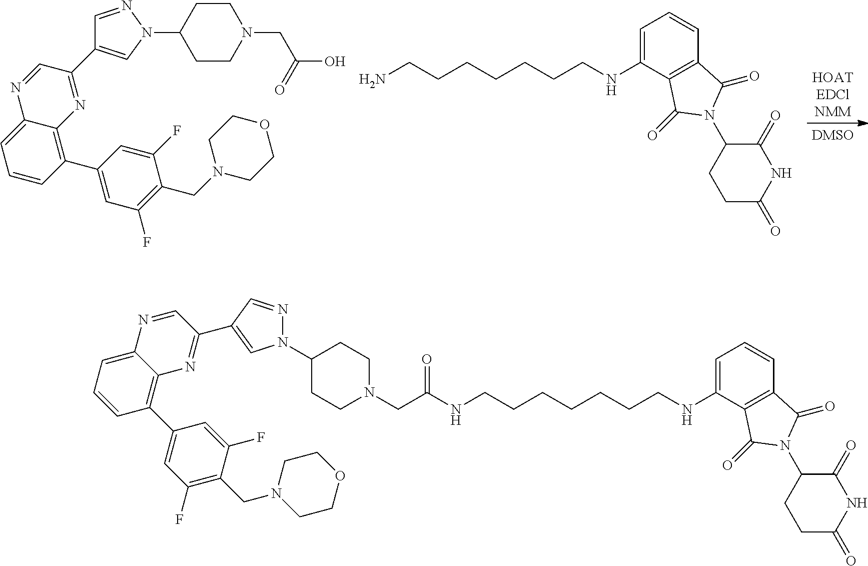 [SMILES: O=C(O)CN1CCC(n2cc(-c3cnc4cccc(-c5cc(F)c(CN6CCOCC6)c(F)c5)c4n3)cn2)CC1 ; NCCCCCCCNc1cccc2c1C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCCCCNc1cccc2c1C(=O)N(C1CCC(=O)NC1=O)C2=O]

JA-148 was synthesized following the standard procedure for preparing JA-001 (8.5 mg, 49.5% yield). MS (ESI) m/z: 917.4 [M+H]⁺.

### Example 237. 2-(4-(4-(8-(3,5-Difluoro-4-(morpholinomethyl)phenyl)quinoxalin-2-yl)-1H-pyrazol-1-yl)piperidin-1-yl)-N-(8-((2-(2,6-dioxopiperidin-3-yl)-1,3-dioxoisoindolin-4-yl)amino)octyl)acetamide (JA-149)

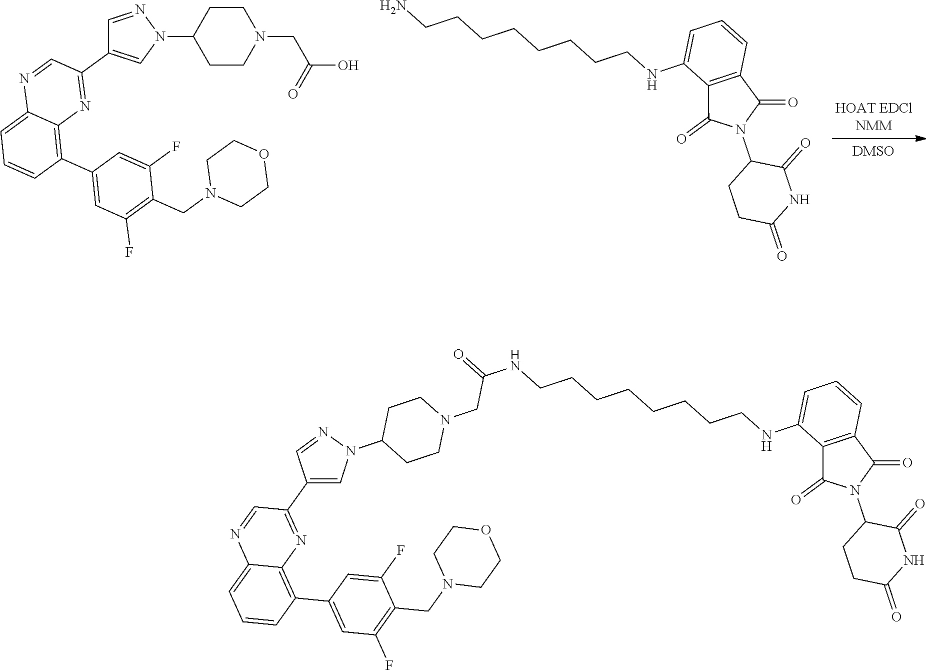

JA-149 was synthesized following the standard procedure for preparing JA-001 (8.9 mg, 51.1% yield). MS (ESI) m/z: 931.4 [M+H]⁺.

### Example 238. (2S,4R)-1-((S)-2-(11-(2-(4-(4-(8-(3,5-Difluoro-4-(morpholinomethyl)phenyl)quinoxalin-2-yl)-1H-pyrazol-1-yl)piperidin-1-yl)acetamido)undecanamido)-3,3-dimethylbutanoyl)-4-hydroxy-N-(4-(4-methylthiazol-5-yl)benzyl)pyrrolidine-2-carboxamide (JA-150)

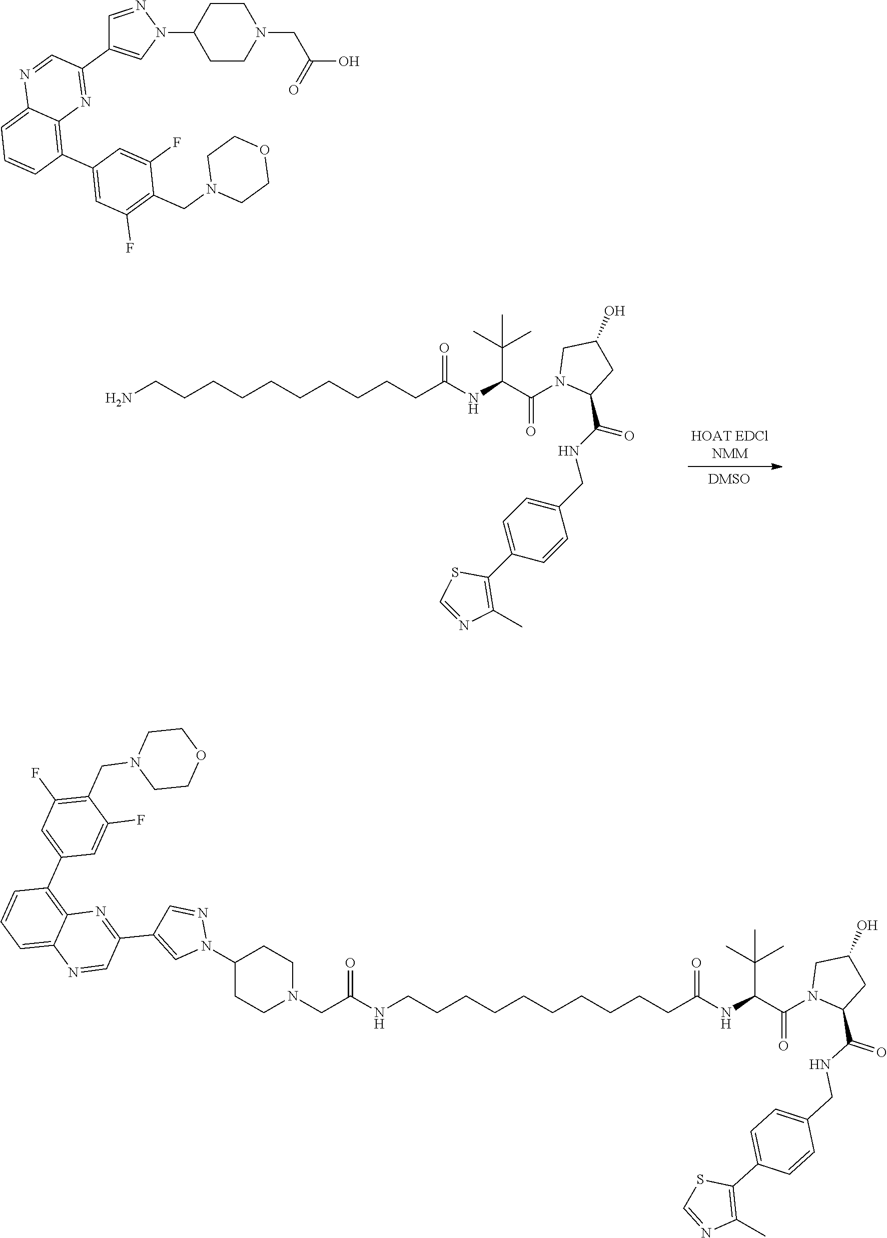 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCCCCCCCCCN)C(C)(C)C)cc1 ; Cc1ncsc1-c1ccc(CNC(=O)[C@@H]2C[C@@H](O)CN2C(=O)[C@@H](NC(=O)CCCCCCCCCCNC(=O)CN2CCC(n3cc(-c4cnc5cccc(-c6cc(F)c(CN7CCOCC7)c(F)c6)c5n4)cn3)CC2)C(C)(C)C)cc1]

JA-150 was synthesized following the standard procedure for preparing JA-001 (10.9 mg, 50.9% yield). MS (ESI) m/z: 1144.6 [M+H]⁺.

### Example 239. (2S,4R)-1-((S)-17-(tert-Butyl)-1-(4-(4-(8-(3,5-difluoro-4-(morpholinomethyl)phenyl)quinoxalin-2-yl)-1H-pyrazol-1-yl)piperidin-1-yl)-2,15-dioxo-6,9,12-trioxa-3,16-diazaoctadecan-18-oyl)-4-hydroxy-N-(4-(4-methylthiazol-5-yl)benzyl)pyrrolidine-2-carboxamide (JA-151)

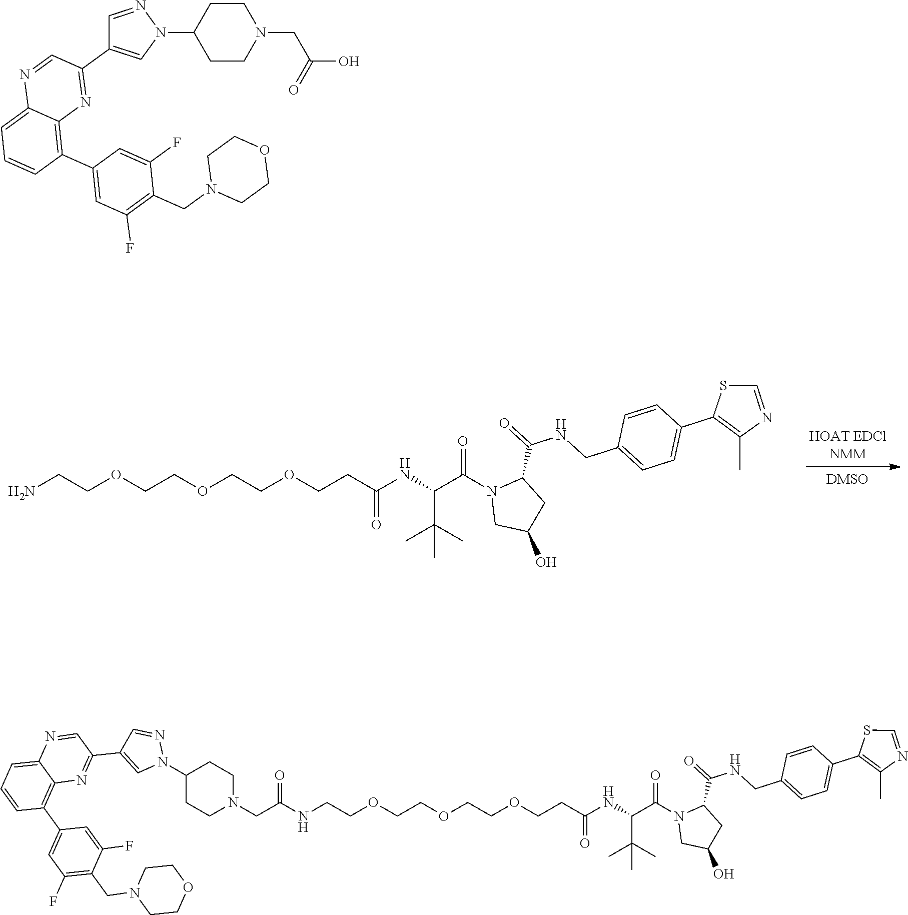 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCOCCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCOCCNC(=O)CN2CCC(n3cc(-c4cnc5cccc(-c6cc(F)c(CN7CCOCC7)c(F)c6)c5n4)cn3)CC2)C(C)(C)C)cc1]

JA-151 was synthesized following the standard procedure for preparing JA-001 (10.2 mg, 46.8% yield). MS (ESI) m/z: 1164.5 [M+H]⁺.

### Example 240. (2S,4R)-1-((S)-2-(4-(2-(4-(4-(8-(3,5-Difluoro-4-(morpholinomethyl)phenyl)quinoxalin-2-yl)-1H-pyrazol-1-yl)piperidin-1-yl)acetamido)butanamido)-3,3-dimethylbutanoyl)-4-hydroxy-N-(4-(4-methylthiazol-5-yl)benzyl)pyrrolidine-2-carboxamide (JA-152)

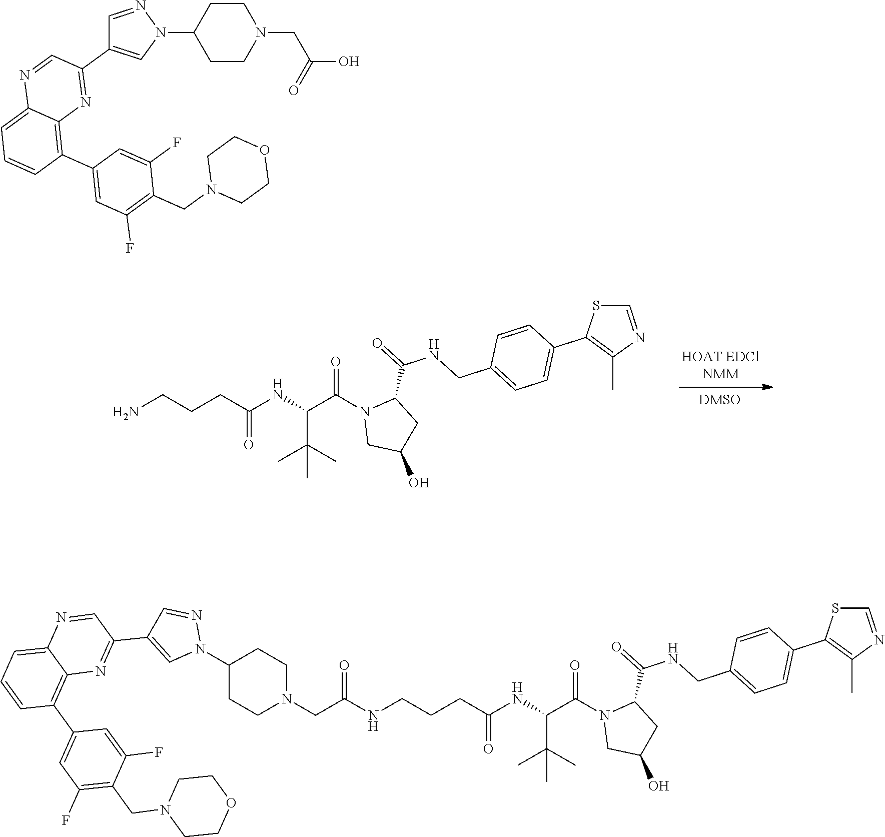 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCCN)C(C)(C)C)cc1 ; Cc1ncsc1-c1ccc(CNC(=O)[C@@H]2C[C@@H](O)CN2C(=O)[C@@H](NC(=O)CCCNC(=O)CN2CCC(n3cc(-c4cnc5cccc(-c6cc(F)c(CN7CCOCC7)c(F)c6)c5n4)cn3)CC2)C(C)(C)C)cc1]

JA-152 was synthesized following the standard procedure for preparing JA-001 (9.4 mg, 48.0% yield). MS (ESI) m/z: 1046.5 [M+H]⁺.

### Example 241. (2S,4R)-1-((S)-2-(2-(2-(2-(4-(4-(8-(3,5-Difluoro-4-(morpholinomethyl)phenyl)quinoxalin-2-yl)-1H-pyrazol-1-yl)piperidin-1-yl)acetamido)ethoxy)acetamido)-3,3-dimethylbutanoyl)-4-hydroxy-N-(4-(4-methylthiazol-5-yl)benzyl)pyrrolidine-2-carboxamide (JA-153)

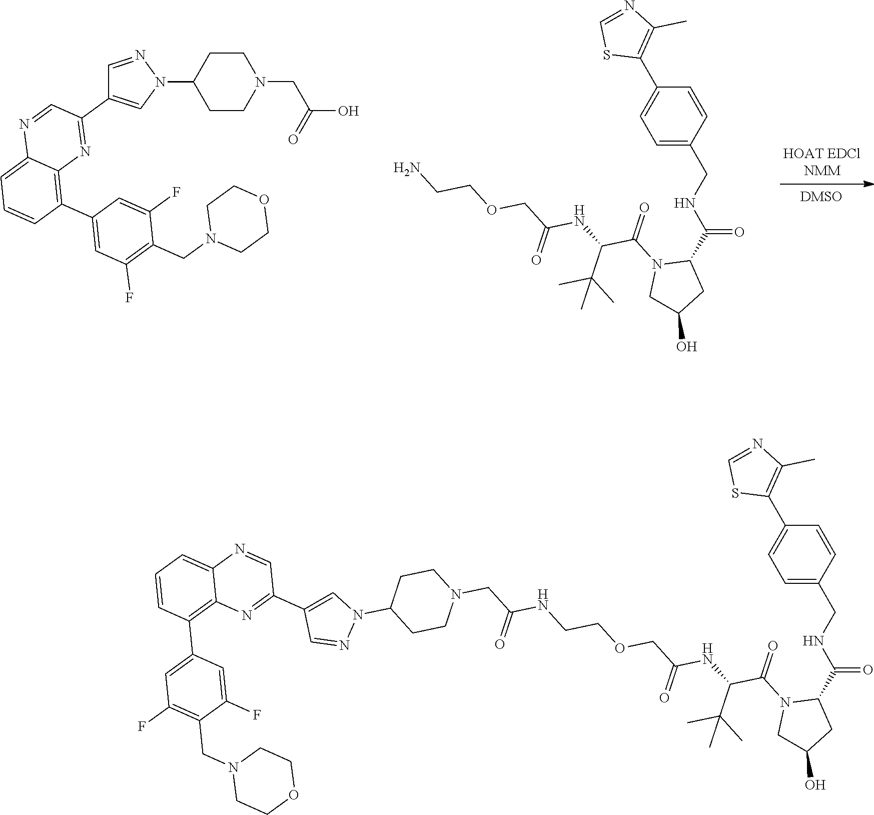

JA-153 was synthesized following the standard procedure for preparing JA-001 (10.3 mg, 51.8% yield). MS (ESI) m/z: 1062.5 [M+H]⁺.

### Example 242. 2-(4-(4-(8-(3,5-Difluoro-4-(morpholinomethyl)phenyl)quinoxalin-2-yl)-1H-pyrazol-1-yl)piperidin-1-yl)-N-(3-((2-(2,6-dioxopiperidin-3-yl)-1,3-dioxoisoindolin-4-yl)amino)propyl)acetamide (JA-154)

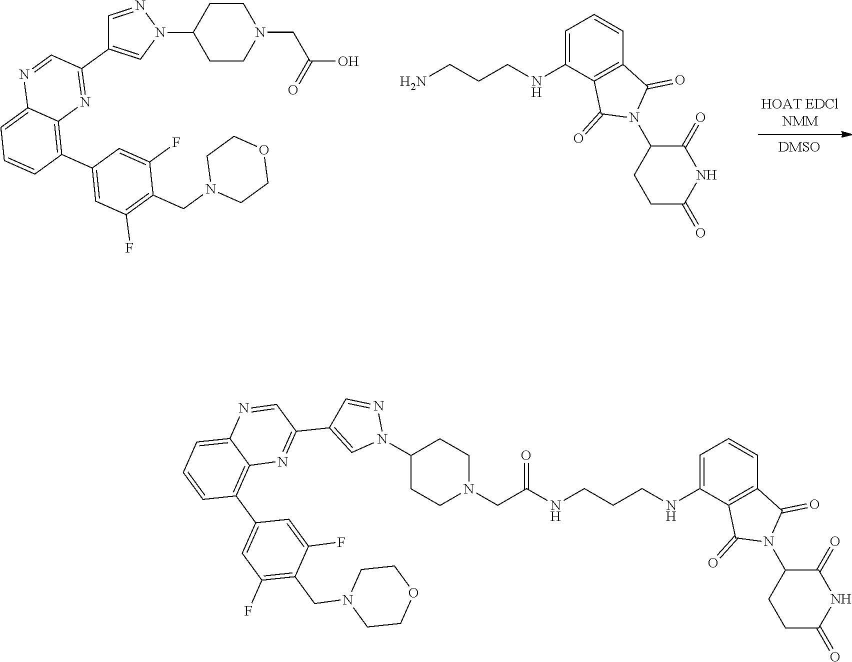

JA-154 was synthesized following the standard procedure for preparing JA-001 (8.9 mg, 55.2% yield). MS (ESI) m/z: 861.4 [M+H]⁺.

### Example 243. (2S,4R)-1-((S)-2-(3-(2-(4-(4-(8-(3,5-Difluoro-4-(morpholinomethyl)phenyl)quinoxalin-2-yl)-1H-pyrazol-1-yl)piperidin-1-yl)acetamido)propanamido)-3,3-dimethylbutanoyl)-4-hydroxy-N-(4-(4-methylthiazol-5-yl)benzyl)pyrrolidine-2-carboxamide (JA-155)

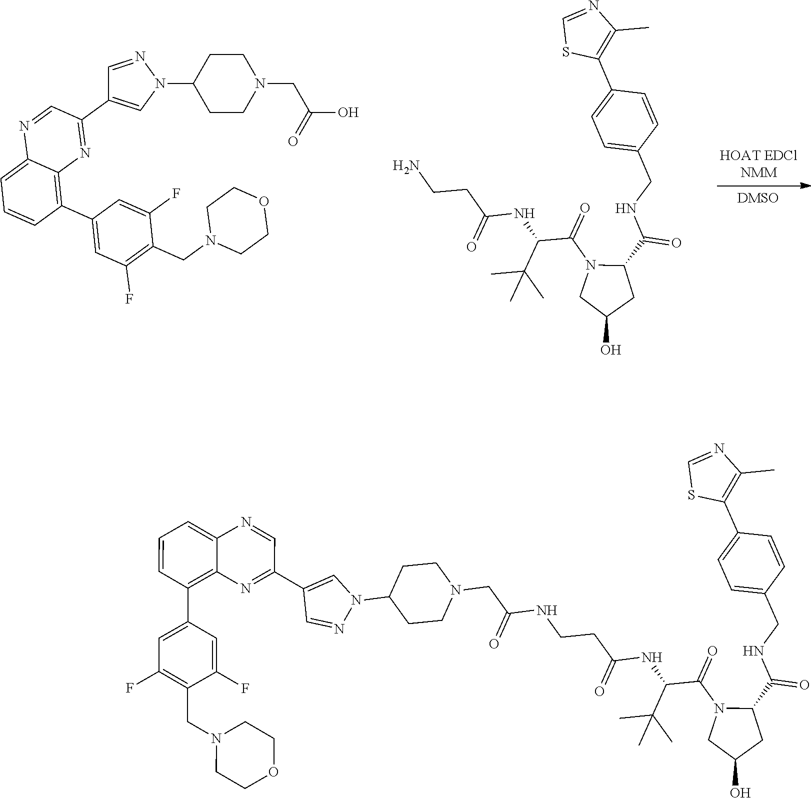

JA-155 was synthesized following the standard procedure for preparing JA-001 (8.5 mg, 44.0% yield). MS (ESI) m/z: 1032.5 [M+H]⁺.

### Example 244. (2S,4R)-1-((S)-2-(8-(2-(4-(4-(8-(3,5-Difluoro-4-(morpholinomethyl)phenyl)quinoxalin-2-yl)-1H-pyrazol-1-yl)piperidin-1-yl)acetamido)octanamido)-3,3-dimethylbutanoyl)-4-hydroxy-N-(4-(4-methylthiazol-5-yl)benzyl)pyrrolidine-2-carboxamide (JA-156)

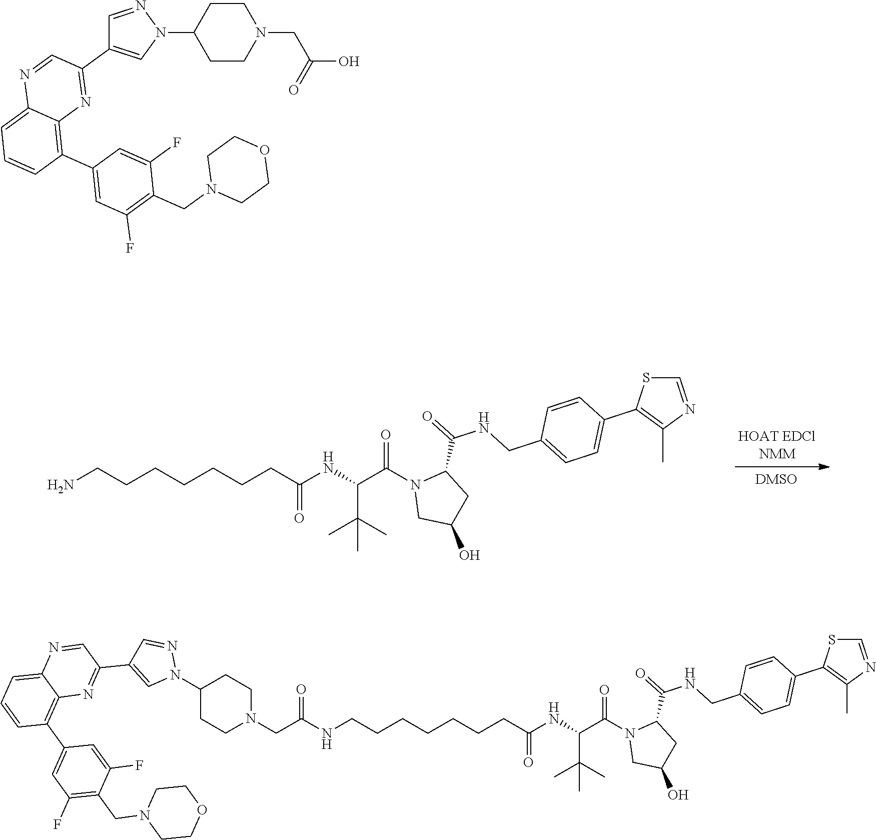 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCCCCCCN)C(C)(C)C)cc1 ; Cc1ncsc1-c1ccc(CNC(=O)[C@@H]2C[C@@H](O)CN2C(=O)[C@@H](NC(=O)CCCCCCCNC(=O)CN2CCC(n3cc(-c4cnc5cccc(-c6cc(F)c(CN7CCOCC7)c(F)c6)c5n4)cn3)CC2)C(C)(C)C)cc1]

JA-156 was synthesized following the standard procedure for preparing JA-001 (10.6 mg, 51.4% yield). MS (ESI) m/z: 1102.5 [M+H]⁺.

### Example 245. (2S,4R)-1-((S)-2-(tert-Butyl)-17-(4-(4-(8-(3,5-Difluoro-4-(morpholinomethyl)phenyl)quinoxalin-2-yl)-1H-pyrazol-1-yl)piperidin-1-yl)-4,16-dioxo-6,9,12-trioxa-3,15-diazaheptadecanoyl)-4-hydroxy-N-(4-(4-methylthiazol-5-yl)benzyl)pyrrolidine-2-carboxamide (JA-157)

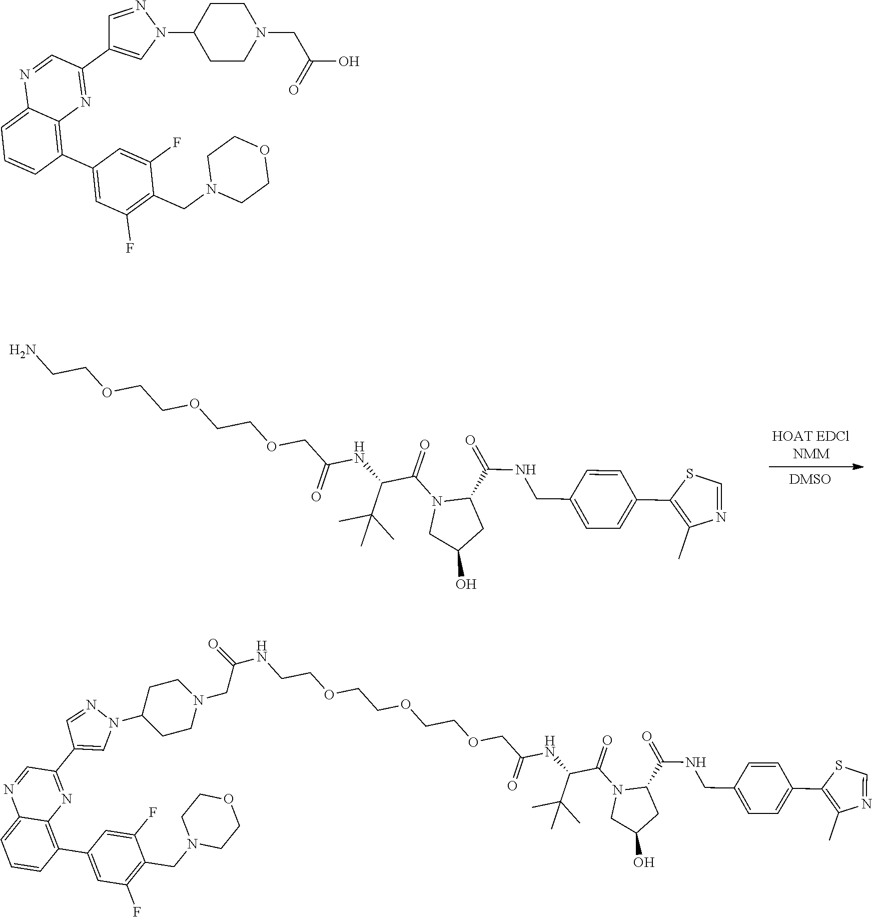 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)COCCOCCOCCN)C(C)(C)C)cc1 ; Cc1ncsc1-c1ccc(CNC(=O)[C@@H]2C[C@@H](O)CN2C(=O)[C@@H](NC(=O)COCCOCCOCCNC(=O)CN2CCC(n3cc(-c4cnc5cccc(-c6cc(F)c(CN7CCOCC7)c(F)c6)c5n4)cn3)CC2)C(C)(C)C)cc1]

JA-157 was synthesized following the standard procedure for preparing JA-001 (10.8 mg, 50.2% yield). MS (ESI) m/z: 1150.5 [M+H]⁺.

### Example 246. (2S,4R)-1-((S)-2-(2-(2-(4-(4-(8-(3,5-Difluoro-4-(morpholinomethyl)phenyl)quinoxalin-2-yl)-1H-pyrazol-1-yl)piperidin-1-yl)acetamido)acetamido)-3,3-dimethylbutanoyl)-4-hydroxy-N-(4-(4-methylthiazol-5-yl)benzyl)pyrrolidine-2-carboxamide (JA-158)

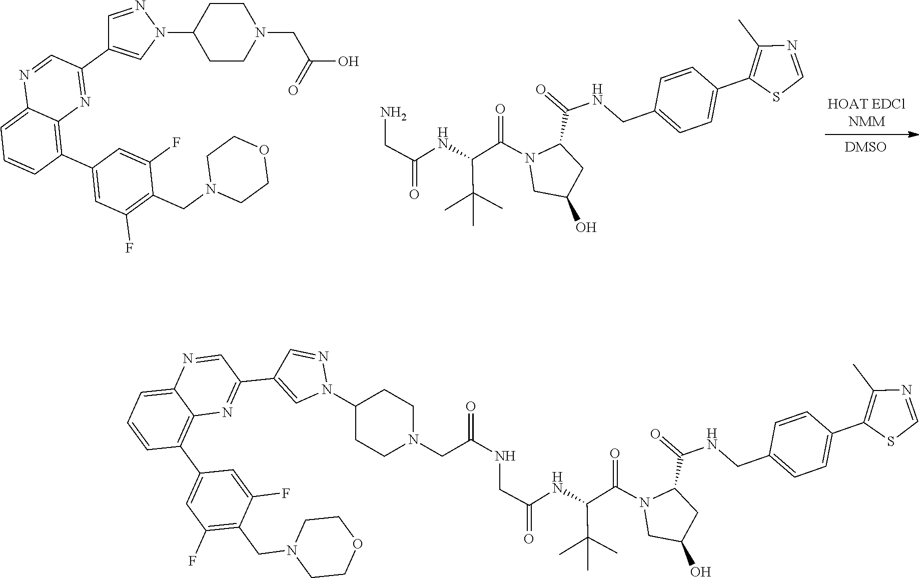 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CN)C(C)(C)C)cc1 ; Cc1ncsc1-c1ccc(CNC(=O)[C@@H]2C[C@@H](O)CN2C(=O)[C@@H](NC(=O)CNC(=O)CN2CCC(n3cc(-c4cnc5cccc(-c6cc(F)c(CN7CCOCC7)c(F)c6)c5n4)cn3)CC2)C(C)(C)C)cc1]

JA-158 was synthesized following the standard procedure for preparing JA-001 (9.2 mg, 48.3% yield). MS (ESI) m/z: 1018.4 [M+H]⁺.

### Example 247. (2S,4R)-1-((S)-23-(tert-Butyl)-1-(4-(4-(8-(3,5-difluoro-4-(morpholinomethyl)phenyl)quinoxalin-2-yl)-1H-pyrazol-1-yl)piperidin-1-yl)-2,21-dioxo-6,9,12,15,18-pentaoxa-3,22-diazatetracosan-24-oyl)-4-hydroxy-N-(4-(4-methylthiazol-5-yl)benzyl)pyrrolidine-2-carboxamide (JA-159)

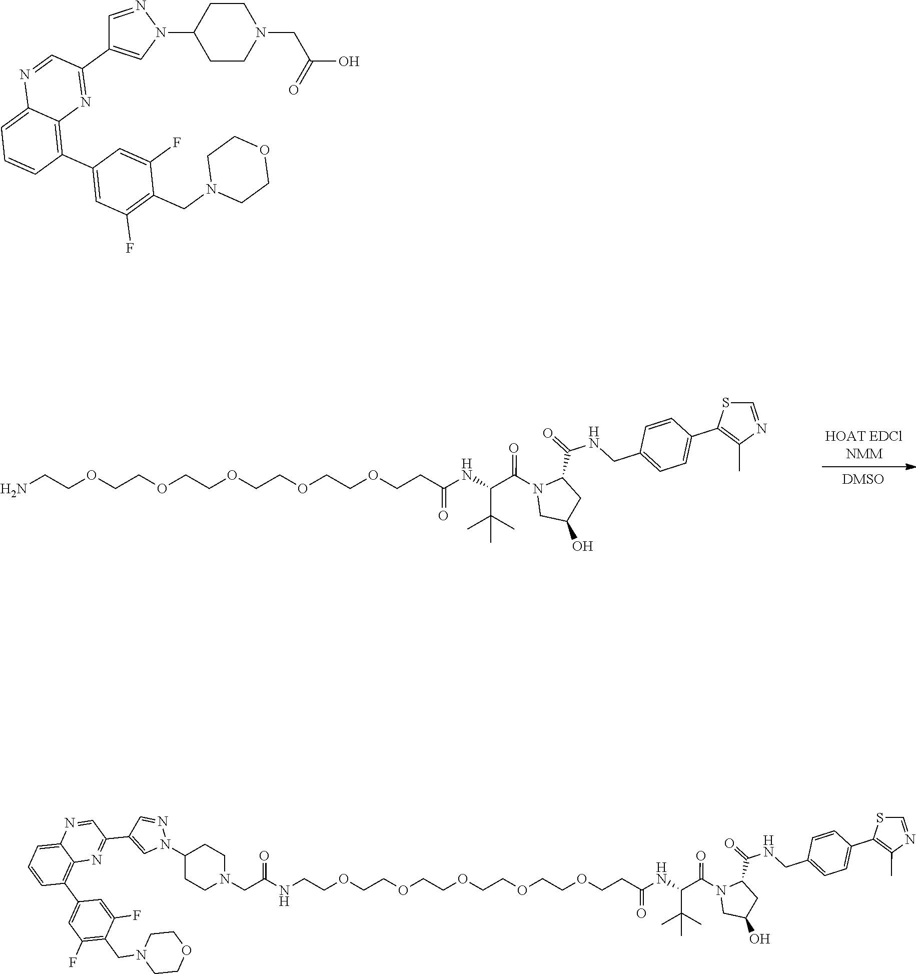 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCOCCOCCOCCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCOCCOCCOCCNC(=O)CN2CCC(n3cc(-c4cnc5cccc(-c6cc(F)c(CN7CCOCC7)c(F)c6)c5n4)cn3)CC2)C(C)(C)C)cc1]

JA-159 was synthesized following the standard procedure for preparing JA-001 (10.6 mg, 45.2% yield). MS (ESI) m/z: 1252.6 [M+H]⁺.

### Example 248. (2S,4R)-1-((S)-2-(3-(2-(2-(4-(4-(8-(3,5-Difluoro-4-(morpholinomethyl)phenyl)quinoxalin-2-yl)-1H-pyrazol-1-yl)piperidin-1-yl)acetamido)ethoxy)propanamido)-3,3-dimethylbutanoyl)-4-hydroxy-N-(4-(4-methylthiazol-5-yl)benzyl)pyrrolidine-2-carboxamide (JA-160)

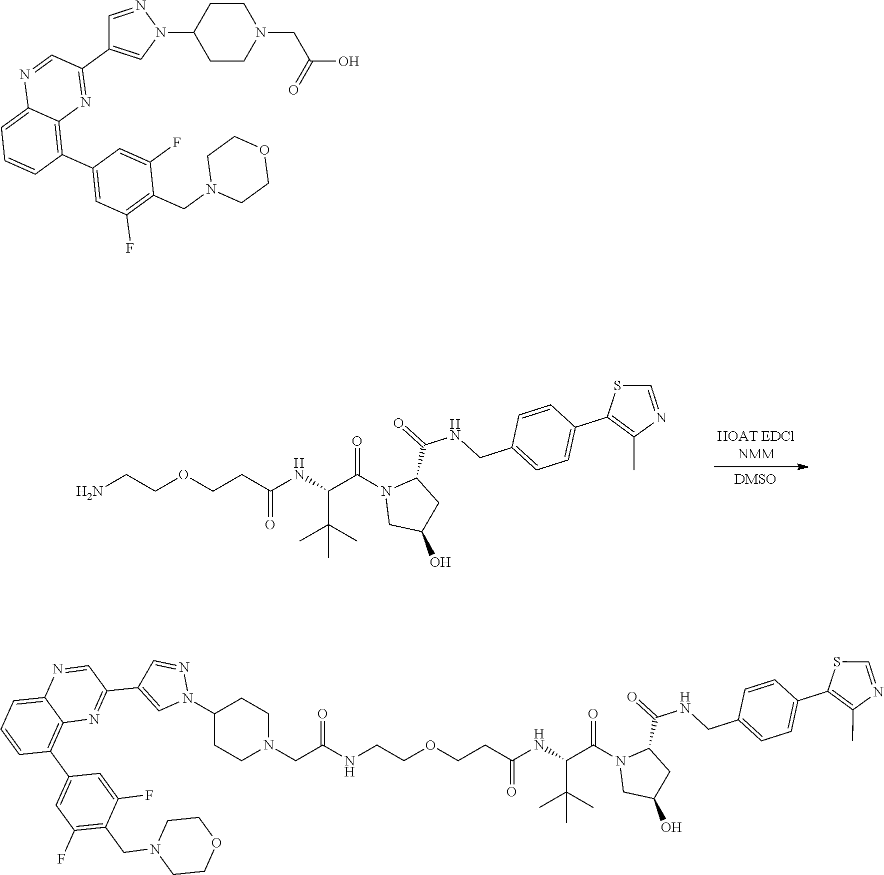 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCOCCN)C(C)(C)C)cc1 ; Cc1ncsc1-c1ccc(CNC(=O)[C@@H]2C[C@@H](O)CN2C(=O)[C@@H](NC(=O)CCOCCNC(=O)CN2CCC(n3cc(-c4cnc5cccc(-c6cc(F)c(CN7CCOCC7)c(F)c6)c5n4)cn3)CC2)C(C)(C)C)cc1]

JA-160 was synthesized following the standard procedure for preparing JA-001 (8.4 mg, 41.7% yield). MS (ESI) m/z: 1076.5 [M+H]⁺.

### Example 249. (2S,4R)-1-((S)-2-(9-(2-(4-(4-(8-(3,5-Difluoro-4-(morpholinomethyl)phenyl)quinoxalin-2-yl)-1H-pyrazol-1-yl)piperidin-1-yl)acetamido)nonanamido)-3,3-dimethylbutanoyl)-4-hydroxy-N-(4-(4-methylthiazol-5-yl)benzyl)pyrrolidine-2-carboxamide (JA-161)

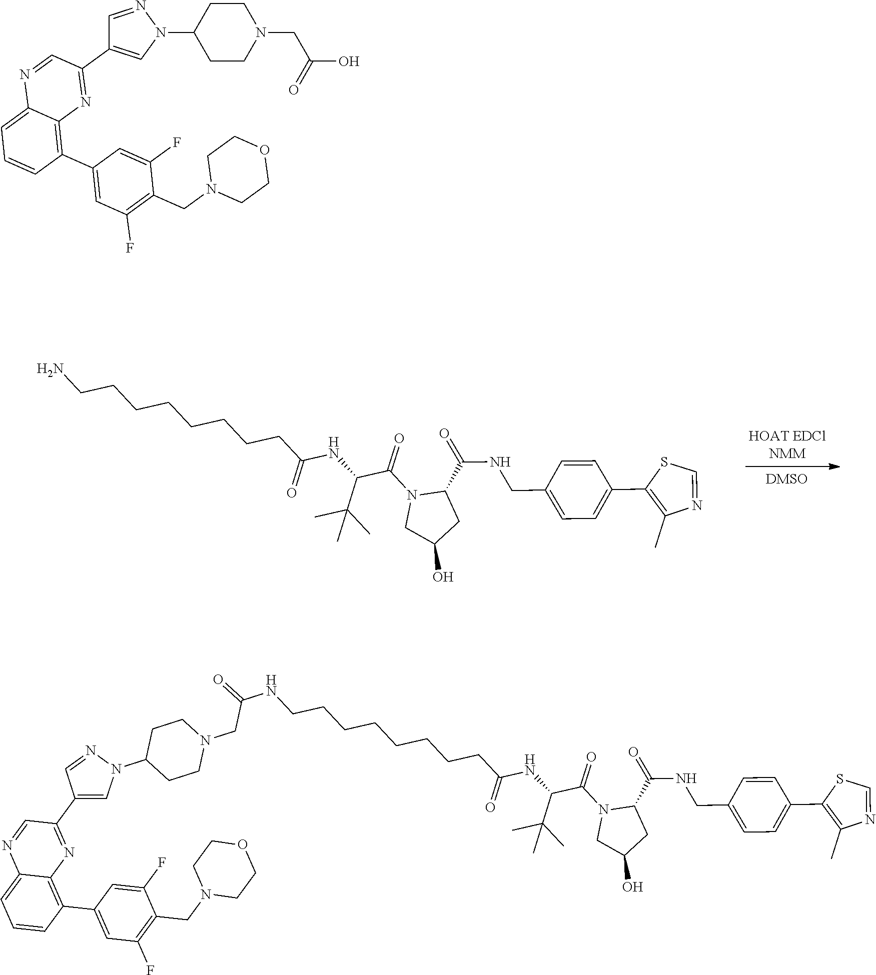 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCCCCCCCN)C(C)(C)C)cc1 ; Cc1ncsc1-c1ccc(CNC(=O)[C@@H]2C[C@@H](O)CN2C(=O)[C@@H](NC(=O)CCCCCCCCNC(=O)CN2CCC(n3cc(-c4cnc5cccc(-c6cc(F)c(CN7CCOCC7)c(F)c6)c5n4)cn3)CC2)C(C)(C)C)cc1]

JA-161 was synthesized following the standard procedure for preparing JA-001 (9.4 mg, 45.0% yield). MS (ESI) m/z: 1116.6 [M+H]⁺.

### Example 250. (2S,4R)-1-((S)-14-(tert-Butyl)-1-(4-(4-(8-(3,5-difluoro-4-(morpholinomethyl)phenyl)quinoxalin-2-yl)-1H-pyrazol-1-yl)piperidin-1-yl)-2,12-dioxo-6,9-dioxa-3,13-diazapentadecan-15-oyl)-4-hydroxy-N-(4-(4-methylthiazol-5-yl)benzyl)pyrrolidine-2-carboxamide (JA-162)

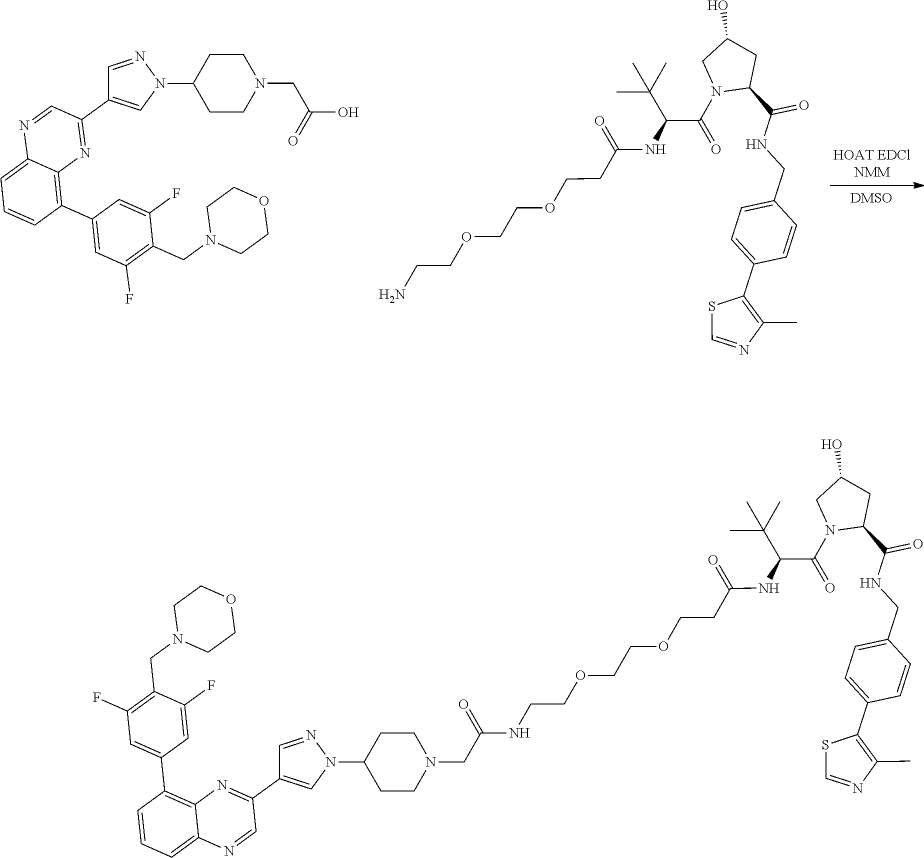

JA-162 was synthesized following the standard procedure for preparing JA-001 (10.6 mg, 50.6% yield). MS (ESI) m/z: 1120.5 [M+H]⁺.

### Example 251. (2S,4R)-1-((S)-2-(tert-Butyl)-14-(4-(4-(8-(3,5-difluoro-4-(morpholinomethyl)phenyl)quinoxalin-2-yl)-1H-pyrazol-1-yl)piperidin-1-yl)-4,13-dioxo-6,9-dioxa-3,12-diazatetradecanoyl)-4-hydroxy-N-(4-(4-methylthiazol-5-yl)benzyl)pyrrolidine-2-carboxamide (JA-163)

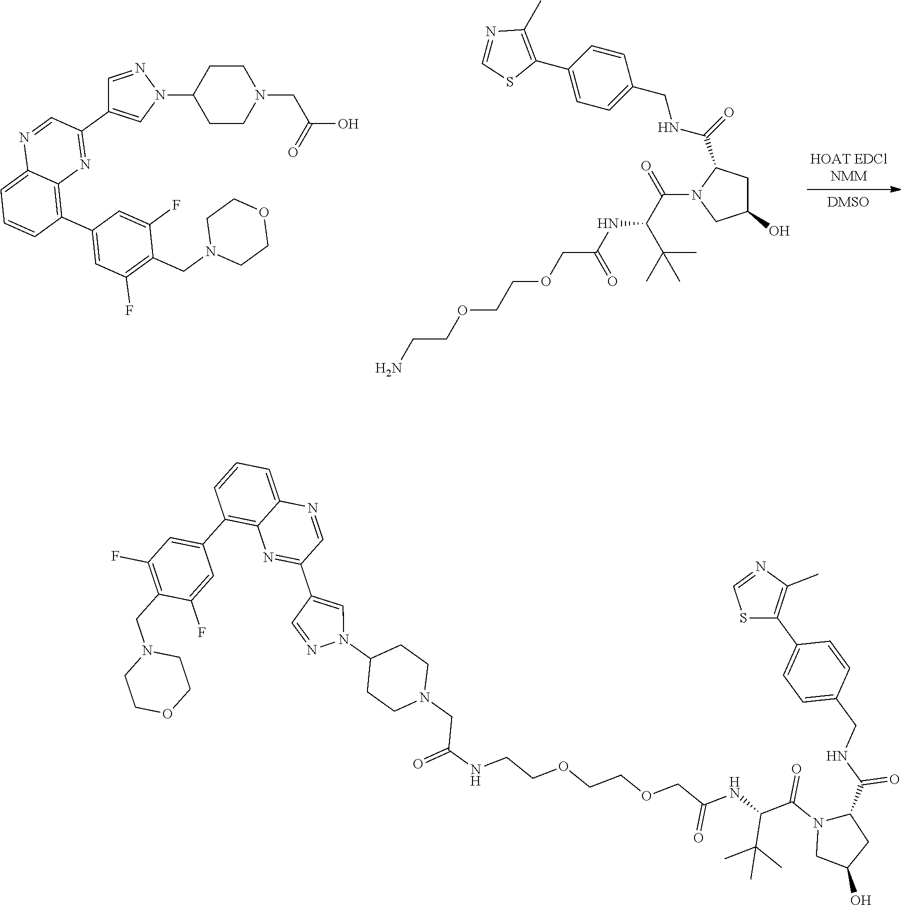 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)COCCOCCN)C(C)(C)C)cc1 ; Cc1ncsc1-c1ccc(CNC(=O)[C@@H]2C[C@@H](O)CN2C(=O)[C@@H](NC(=O)COCCOCCNC(=O)CN2CCC(n3cc(-c4cnc5cccc(-c6cc(F)c(CN7CCOCC7)c(F)c6)c5n4)cn3)CC2)C(C)(C)C)cc1]

JA-163 was synthesized following the standard procedure for preparing JA-001 (10.8 mg, 52.2% yield). MS (ESI) m/z: 1106.5 [M+H]⁺.

### Example 252. (2S,4R)-1-((S)-2-(6-(2-(4-(4-(8-(3,5-Difluoro-4-(morpholinomethyl)phenyl)quinoxalin-2-yl)-1H-pyrazol-1-yl)piperidin-1-yl)acetamido)hexanamido)-3,3-dimethylbutanoyl)-4-hydroxy-N-(4-(4-methylthiazol-5-yl)benzyl)pyrrolidine-2-carboxamide (JA-164)

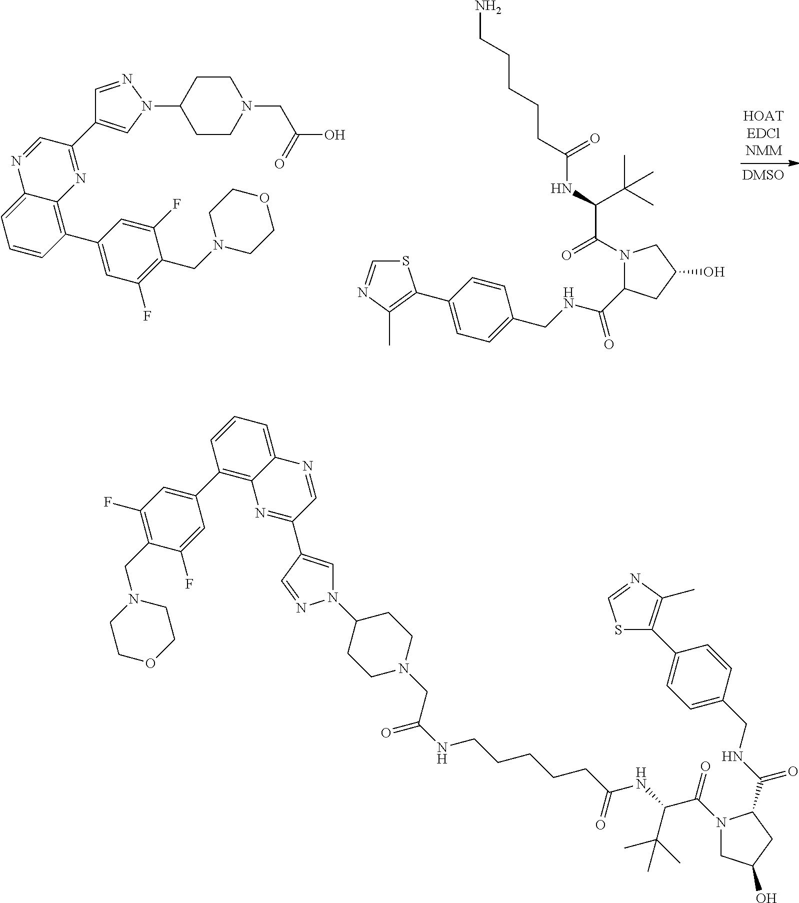 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)C2C[C@@H](O)CN2C(=O)[C@@H](NC(=O)CCCCCN)C(C)(C)C)cc1 ; Cc1ncsc1-c1ccc(CNC(=O)[C@@H]2C[C@@H](O)CN2C(=O)[C@@H](NC(=O)CCCCCNC(=O)CN2CCC(n3cc(-c4cnc5cccc(-c6cc(F)c(CN7CCOCC7)c(F)c6)c5n4)cn3)CC2)C(C)(C)C)cc1]

JA-164 was synthesized following the standard procedure for preparing JA-001 (9.8 mg, 48.7% yield). MS (ESI) m/z: 1074.5 [M+H]⁺.

### Example 253. (2S,4R)-1-((S)-2-(7-(2-(4-(4-(8-(3,5-Difluoro-4-(morpholinomethyl)phenyl)quinoxalin-2-yl)-1H-pyrazol-1-yl)piperidin-1-yl)acetamido)heptanamido)-3,3-dimethylbutanoyl)-4-hydroxy-N-(4-(4-methylthiazol-5-yl)benzyl)pyrrolidine-2-carboxamide (JA-165)

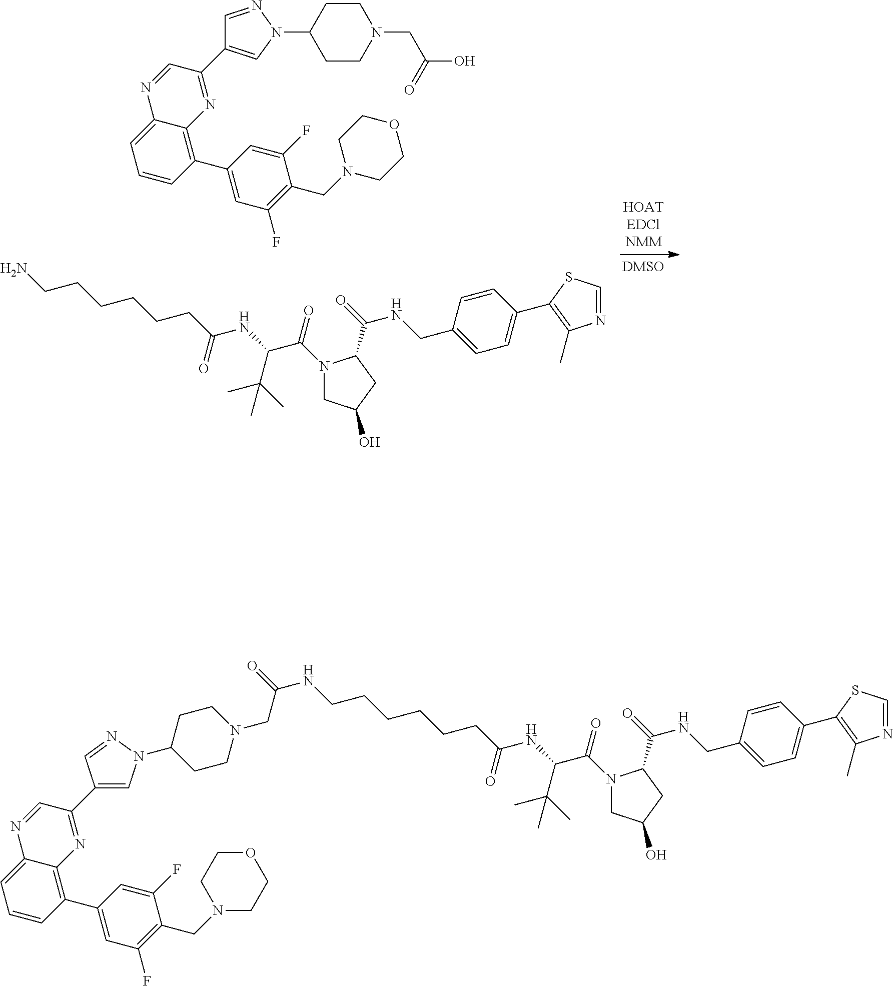 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCCCCCN)C(C)(C)C)cc1 ; Cc1ncsc1-c1ccc(CNC(=O)[C@@H]2C[C@@H](O)CN2C(=O)[C@@H](NC(=O)CCCCCCNC(=O)CN2CCC(n3cc(-c4cnc5cccc(-c6cc(F)c(CN7CCOCC7)c(F)c6)c5n4)cn3)CC2)C(C)(C)C)cc1]

JA-165 was synthesized following the standard procedure for preparing JA-001 (9.5 mg, 46.6% yield). MS (ESI) m/z: 1088.5 [M+H]⁺.

### Example 254. (2S,4R)-1-((S)-2-(10-(2-(4-(4-(8-(3,5-Difluoro-4-(morpholinomethyl)phenyl)quinoxalin-2-yl)-1H-pyrazol-1-yl)piperidin-1-yl)acetamido)decanamido)-3,3-dimethylbutanoyl)-4-hydroxy-N-(4-(4-methylthiazol-5-yl)benzyl)pyrrolidine-2-carboxamide (JA-166)

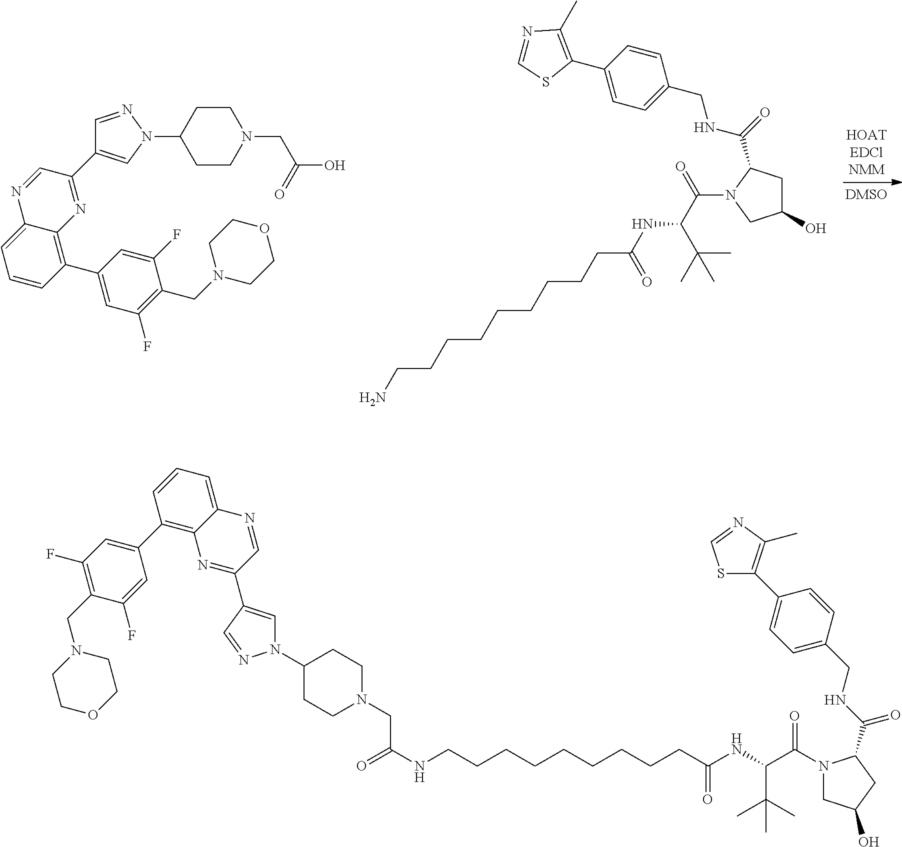 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCCCCCCCCN)C(C)(C)C)cc1 ; Cc1ncsc1-c1ccc(CNC(=O)[C@@H]2C[C@@H](O)CN2C(=O)[C@@H](NC(=O)CCCCCCCCCNC(=O)CN2CCC(n3cc(-c4cnc5cccc(-c6cc(F)c(CN7CCOCC7)c(F)c6)c5n4)cn3)CC2)C(C)(C)C)cc1]

JA-166 was synthesized following the standard procedure for preparing JA-001 (9.6 mg, 45.4% yield). MS (ESI) m/z: 1130.6 [M+H]⁺.

### Example 255. (2S,4R)-1-((S)-20-(tert-Butyl)-1-(4-(4-(8-(3,5-difluoro-4-(morpholinomethyl)phenyl)quinoxalin-2-yl)-1H-pyrazol-1-yl)piperidin-1-yl)-2,18-dioxo-6,9,12,15-tetraoxa-3,19-diazahenicosan-21-oyl)-4-hydroxy-N-(4-(4-methylthiazol-5-yl)benzyl)pyrrolidine-2-carboxamide (JA-167)

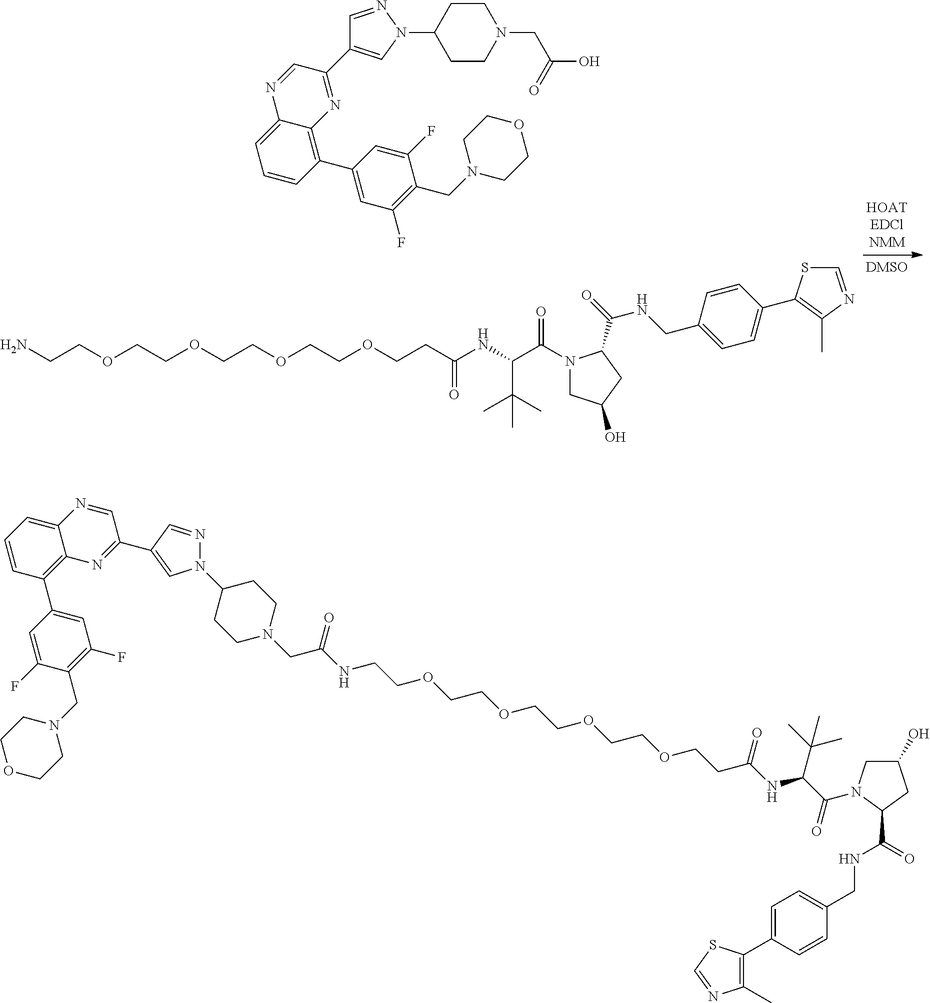 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; Cc1ncsc1-c1ccc(CNC(=O)[C@@H]2C[C@@H](O)CN2C(=O)[C@@H](NC(=O)CCOCCOCCOCCOCCNC(=O)CN2CCC(n3cc(-c4cnc5cccc(-c6cc(F)c(CN7CCOCC7)c(F)c6)c5n4)cn3)CC2)C(C)(C)C)cc1]

JA-167 was synthesized following the standard procedure for preparing JA-001 (9.5 mg, 42.0% yield). MS (ESI) m/z: 1208.6 [M+H]⁺.

### Example 256. (2S,4R)-1-((S)-2-(5-(2-(4-(4-(8-(3,5-Difluoro-4-(morpholinomethyl)phenyl)quinoxalin-2-yl)-1H-pyrazol-1-yl)piperidin-1-yl)acetamido)pentanamido)-3,3-dimethylbutanoyl)-4-hydroxy-N-(4-(4-methylthiazol-5-yl)benzyl)pyrrolidine-2-carboxamide (JA-168)

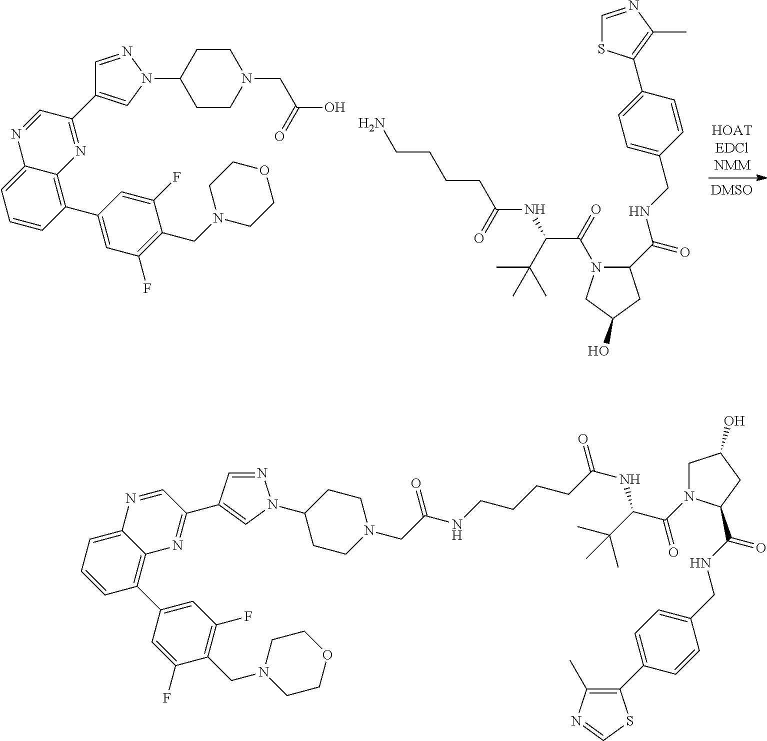 [SMILES: O=C(O)CN1CCC(n2cc(-c3cnc4cccc(-c5cc(F)c(CN6CCOCC6)c(F)c5)c4n3)cn2)CC1 ; Cc1ncsc1-c1ccc(CNC(=O)C2C[C@@H](O)CN2C(=O)[C@@H](NC(=O)CCCCN)C(C)(C)C)cc1 ; Cc1ncsc1-c1ccc(CNC(=O)[C@@H]2C[C@@H](O)CN2C(=O)[C@@H](NC(=O)CCCCNC(=O)CN2CCC(n3cc(-c4cnc5cccc(-c6cc(F)c(CN7CCOCC7)c(F)c6)c5n4)cn3)CC2)C(C)(C)C)cc1]

JA-168 was synthesized following the standard procedure for preparing JA-001 (9.8 mg, 49.4% yield). MS (ESI) m/z: 1060.5 [M+H]⁺.

### Example 257. 2-(4-(4-(8-(3,5-Difluoro-4-(morpholinomethyl)phenyl)quinoxalin-2-yl)-1H-pyrazol-1-yl)piperidin-1-yl)-N-(2-(2-((2-(2,6-dioxopiperidin-3-yl)-1,3-dioxoisoindolin-5-yl)amino)ethoxy)ethyl)acetamide (JA-169)

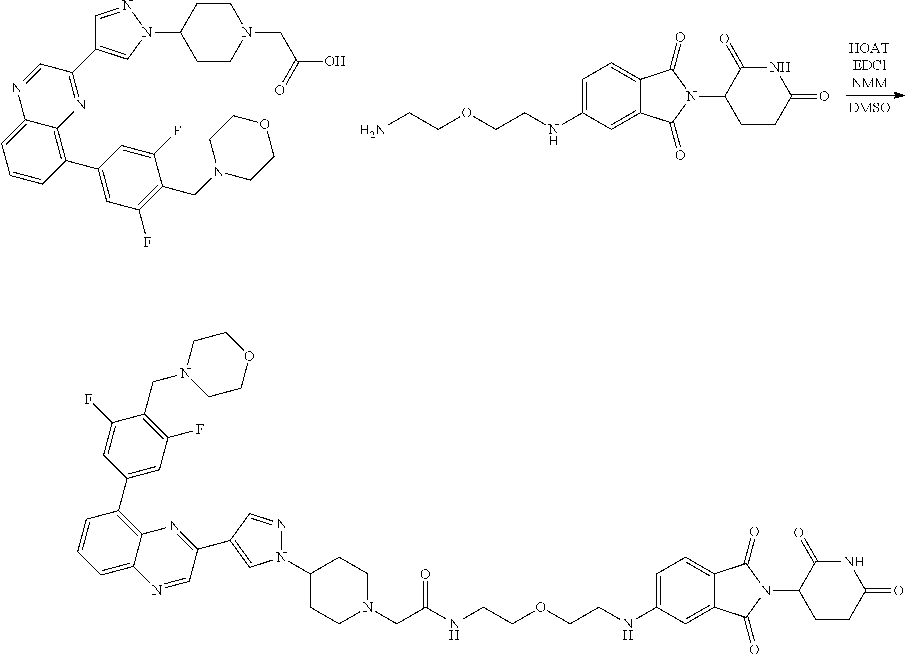 [SMILES: O=C(O)CN1CCC(n2cc(-c3cnc4cccc(-c5cc(F)c(CN6CCOCC6)c(F)c5)c4n3)cn2)CC1 ; NCCOCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCNc1ccc2c(c1)C(=O)N(C1CCC(=O)NC1=O)C2=O]

JA-169 was synthesized following the standard procedure for preparing JA-001 (6.3 mg, 34.1% yield). MS (ESI) m/z: 891.4 [M+H]⁺.

### Example 258. 2-(4-(4-(8-(3,5-Difluoro-4-(morpholinomethyl)phenyl)quinoxalin-2-yl)-1H-pyrazol-1-yl)piperidin-1-yl)-N-(2-(2-(2-((2-(2,6-dioxopiperidin-3-yl)-1,3-dioxoisoindolin-5-yl)amino)ethoxy)ethoxy)ethyl)acetamide (JA-170)

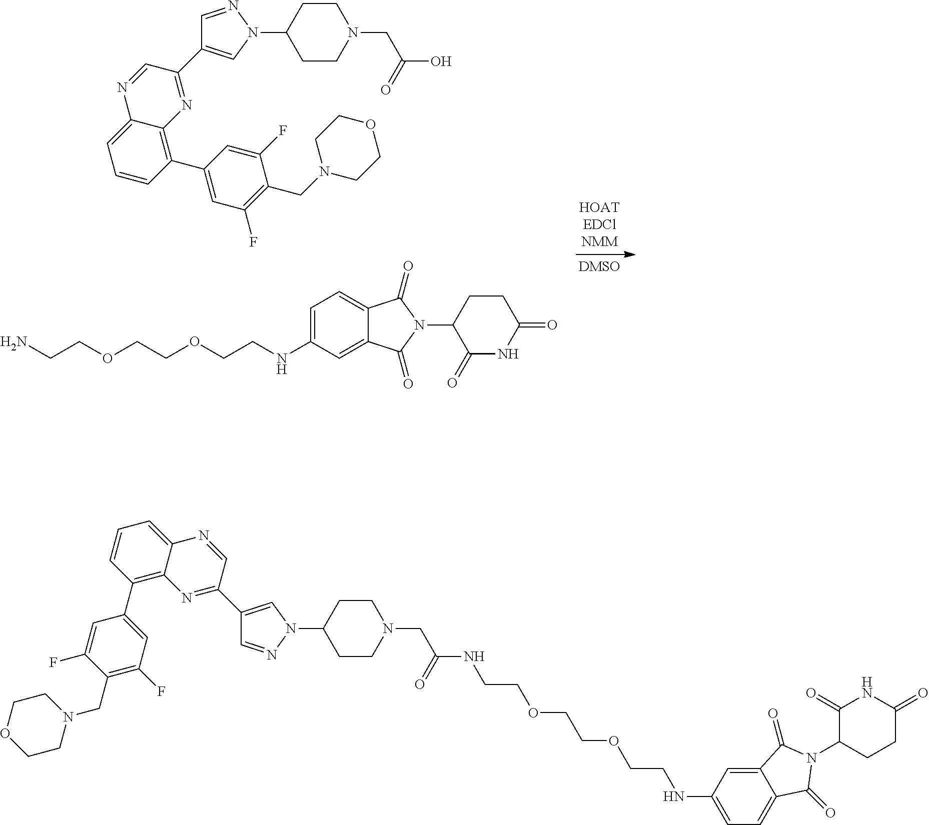 [SMILES: O=C(O)CN1CCC(n2cc(-c3cnc4cccc(-c5cc(F)c(CN6CCOCC6)c(F)c5)c4n3)cn2)CC1 ; NCCOCCOCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCNc1ccc2c(c1)C(=O)N(C1CCC(=O)NC1=O)C2=O]

JA-170 was synthesized following the standard procedure for preparing JA-001 (7.1 mg, 37.6% yield). MS (ESI) m/z: 935.4 [M+H]⁺.

### Example 259. 2-(4-(4-(8-(3,5-Difluoro-4-(morpholinomethyl)phenyl)quinoxalin-2-yl)-1H-pyrazol-1-yl)piperidin-1-yl)-N-(2-(2-(2-(2-((2-(2,6-dioxopiperidin-3-yl)-1,3-dioxoisoindolin-5-yl)amino)ethoxy)ethoxy)ethoxy)ethyl)acetamide (JA-171)

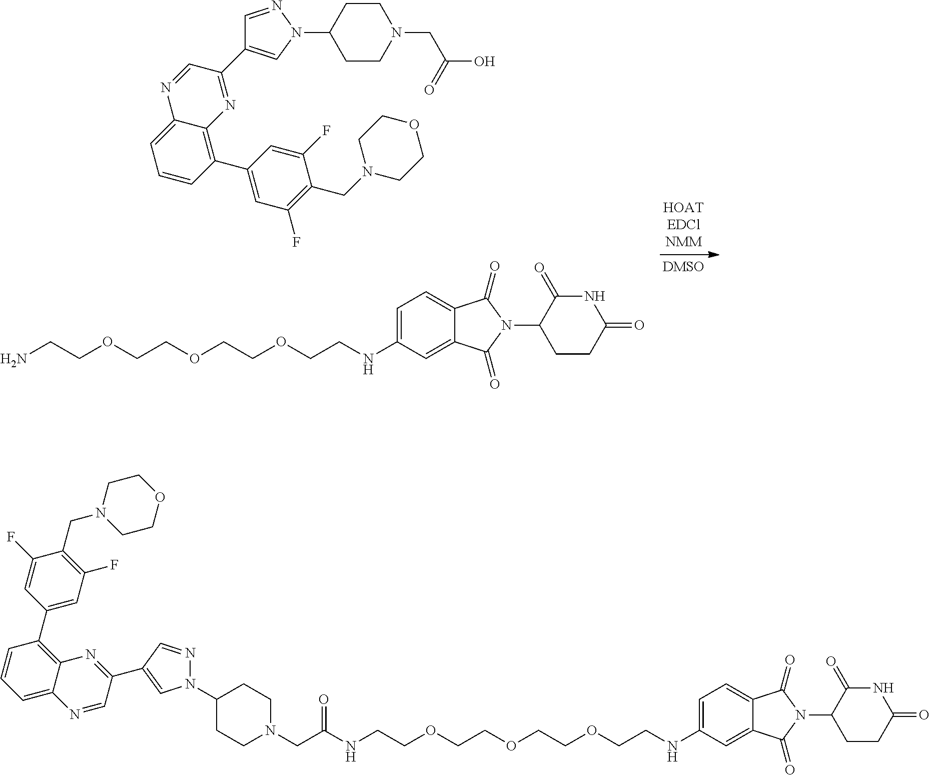 [SMILES: O=C(O)CN1CCC(n2cc(-c3cnc4cccc(-c5cc(F)c(CN6CCOCC6)c(F)c5)c4n3)cn2)CC1 ; NCCOCCOCCOCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCOCCNc1ccc2c(c1)C(=O)N(C1CCC(=O)NC1=O)C2=O]

JA-171 was synthesized following the standard procedure for preparing JA-001 (5.6 mg, 30% yield). MS (ESI) m/z: 979.4 [M+H]⁺.

### Example 260. 2-(4-(4-(8-(3,5-Difluoro-4-(morpholinomethyl)phenyl)quinoxalin-2-yl)-1H-pyrazol-1-yl)piperidin-1-yl)-N-(14-((2-(2,6-dioxopiperidin-3-yl)-1,3-dioxoisoindolin-5-yl)amino)-3,6,9,12-tetraoxatetradecyl)acetamide (JA-172)

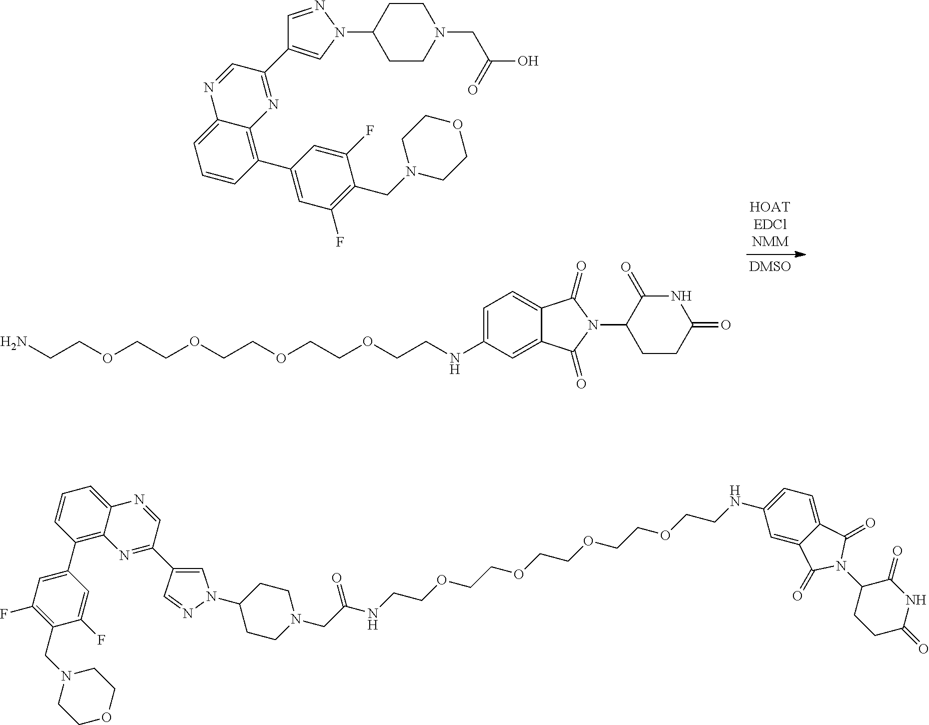 [SMILES: O=C(O)CN1CCC(n2cc(-c3cnc4cccc(-c5cc(F)c(CN6CCOCC6)c(F)c5)c4n3)cn2)CC1 ; NCCOCCOCCOCCOCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCOCCOCCNc1ccc2c(c1)C(=O)N(C1CCC(=O)NC1=O)C2=O]

JA-172 was synthesized following the standard procedure for preparing JA-001 (5.1 mg, 27.2% yield). MS (ESI) m/z: 1023.4 [M+H]⁺.

### Example 261. 2-(4-(4-(8-(3,5-Difluoro-4-(morpholinomethyl)phenyl)quinoxalin-2-yl)-1H-pyrazol-1-yl)piperidin-1-yl)-N-(17-((2-(2,6-dioxopiperidin-3-yl)-1,3-dioxoisoindolin-5-yl)amino)-3,6,9,12,15-pentaoxaheptadecyl)acetamide (JA-173)

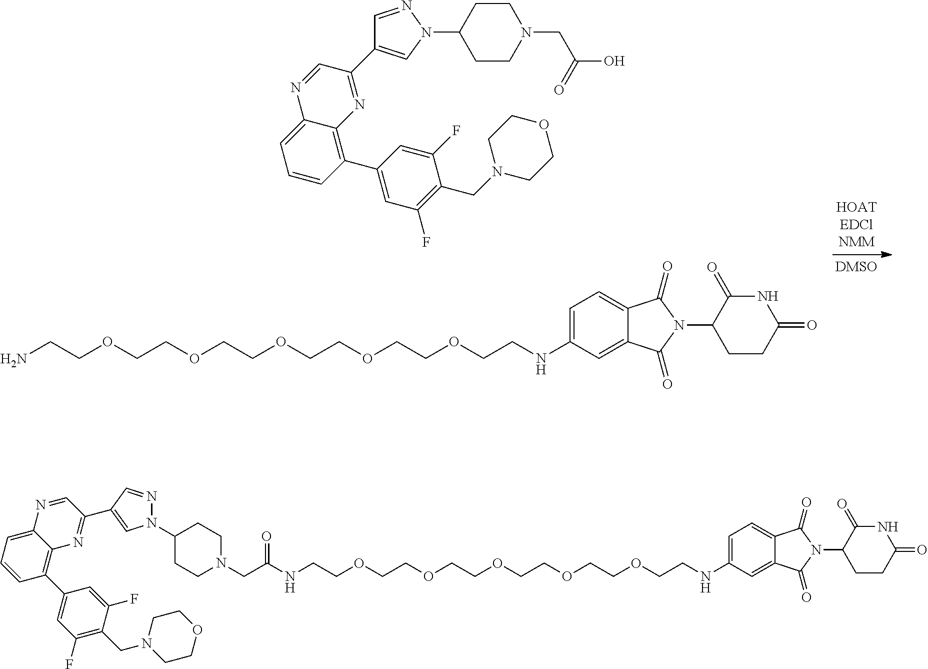 [SMILES: O=C(O)CN1CCC(n2cc(-c3cnc4cccc(-c5cc(F)c(CN6CCOCC6)c(F)c5)c4n3)cn2)CC1 ; NCCOCCOCCOCCOCCOCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCOCCOCCOCCOCCOCCNc1ccc2c(c1)C(=O)N(C1CCC(=O)NC1=O)C2=O]

JA-173 was synthesized following the standard procedure for preparing JA-001 (6.9 mg, 37.7% yield). MS (ESI) m/z: 1067.5 [M+H]⁺.

### Example 262. 2-(4-(4-(8-(3,5-Difluoro-4-(morpholinomethyl)phenyl)quinoxalin-2-yl)-1H-pyrazol-1-yl)piperidin-1-yl)-N-(2-((2-(2,6-dioxopiperidin-3-yl)-1,3-dioxoisoindolin-5-yl)amino)ethyl)acetamide (JA-174)

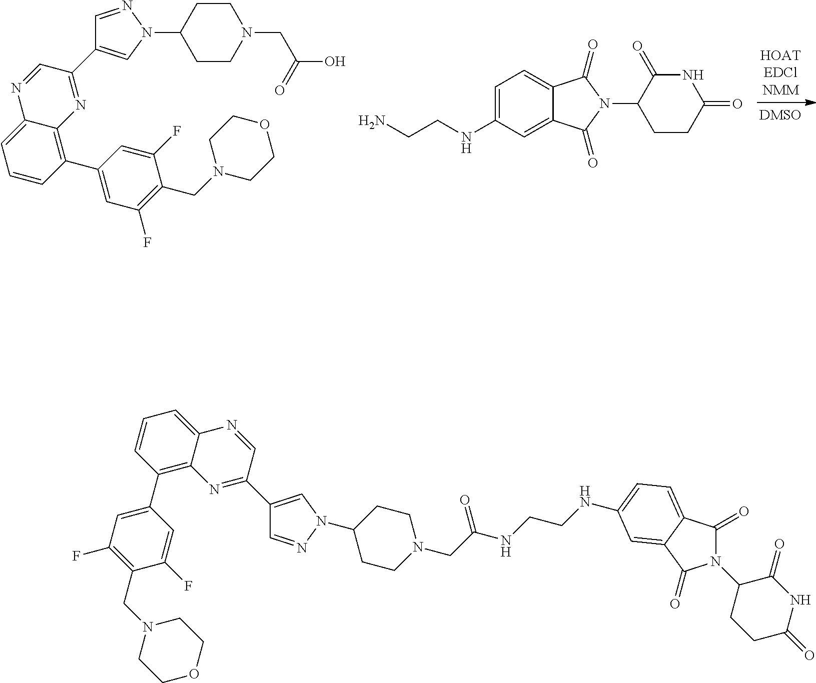 [SMILES: O=C(O)CN1CCC(n2cc(-c3cnc4cccc(-c5cc(F)c(CN6CCOCC6)c(F)c5)c4n3)cn2)CC1 ; NCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCNc1ccc2c(c1)C(=O)N(C1CCC(=O)NC1=O)C2=O]

JA-174 was synthesized following the standard procedure for preparing JA-001 (7.1 mg, 40.4% yield). MS (ESI) m/z: 847.3 [M+H]⁺.

### Example 263. 2-(4-(4-(8-(3,5-Difluoro-4-(morpholinomethyl)phenyl)quinoxalin-2-yl)-1H-pyrazol-1-yl)piperidin-1-yl)-N-(3-((2-(2,6-dioxopiperidin-3-yl)-1,3-dioxoisoindolin-5-yl)amino)propyl)acetamide (JA-175)

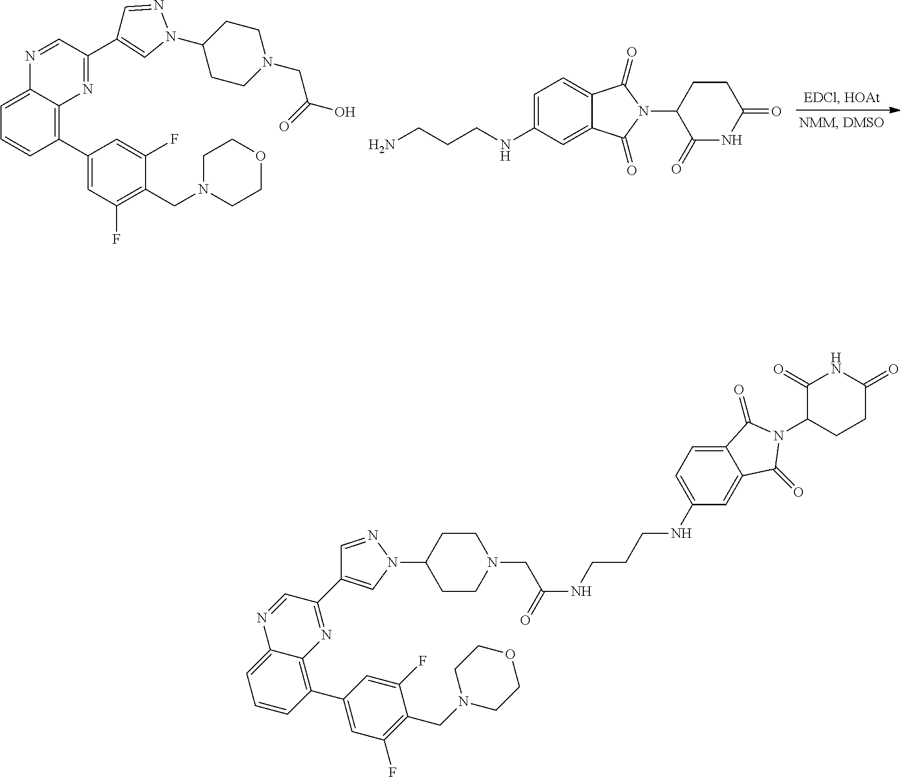 [SMILES: O=C(O)CN1CCC(n2cc(-c3cnc4cccc(-c5cc(F)c(CN6CCOCC6)c(F)c5)c4n3)cn2)CC1 ; NCCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCNc1ccc2c(c1)C(=O)N(C1CCC(=O)NC1=O)C2=O]

JA-175 was synthesized following the standard procedure for preparing JA-001 (5.9 mg, 38.4% yield). MS (ESI) m/z: 861.4 [M+H]⁺.

### Example 264. 2-(4-(4-(8-(3,5-Difluoro-4-(morpholinomethyl)phenyl)quinoxalin-2-yl)-1H-pyrazol-1-yl)piperidin-1-yl)-N-(4-((2-(2,6-dioxopiperidin-3-yl)-1,3-dioxoisoindolin-5-yl)amino)butyl)acetamide (JA-176)

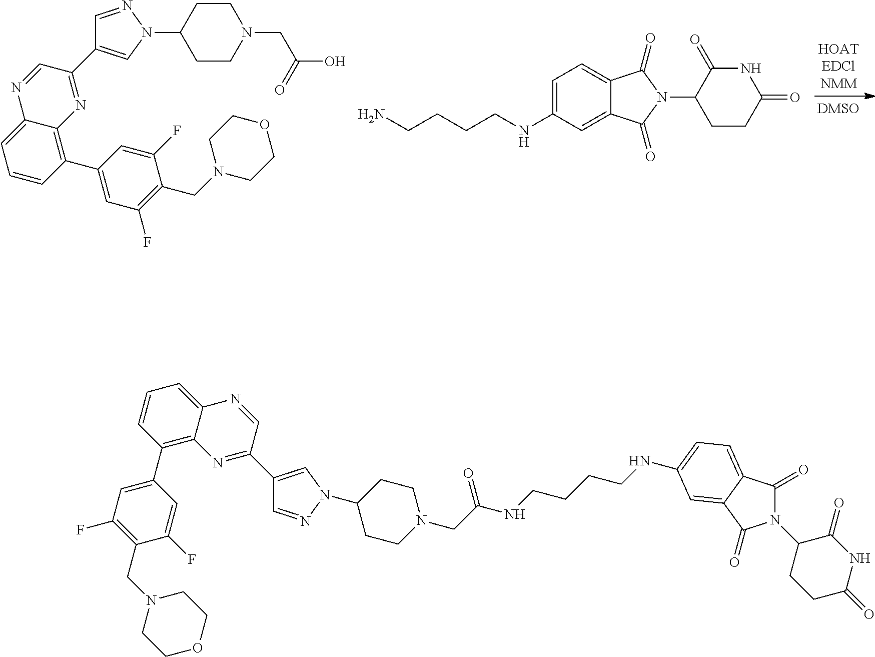 [SMILES: O=C(O)CN1CCC(n2cc(-c3cnc4cccc(-c5cc(F)c(CN6CCOCC6)c(F)c5)c4n3)cn2)CC1 ; NCCCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCNc1ccc2c(c1)C(=O)N(C1CCC(=O)NC1=O)C2=O]

JA-176 was synthesized following the standard procedure for preparing JA-001 (6.9 mg, 40.3% yield). MS (ESI) m/z: 875.4 [M+H]⁺.

### Example 265. 2-(4-(4-(8-(3,5-Difluoro-4-(morpholinomethyl)phenyl)quinoxalin-2-yl)-1H-pyrazol-1-yl)piperidin-1-yl)-N-(5-((2-(2,6-dioxopiperidin-3-yl)-1,3-dioxoisoindolin-5-yl)amino)pentyl)acetamide (JA-177)

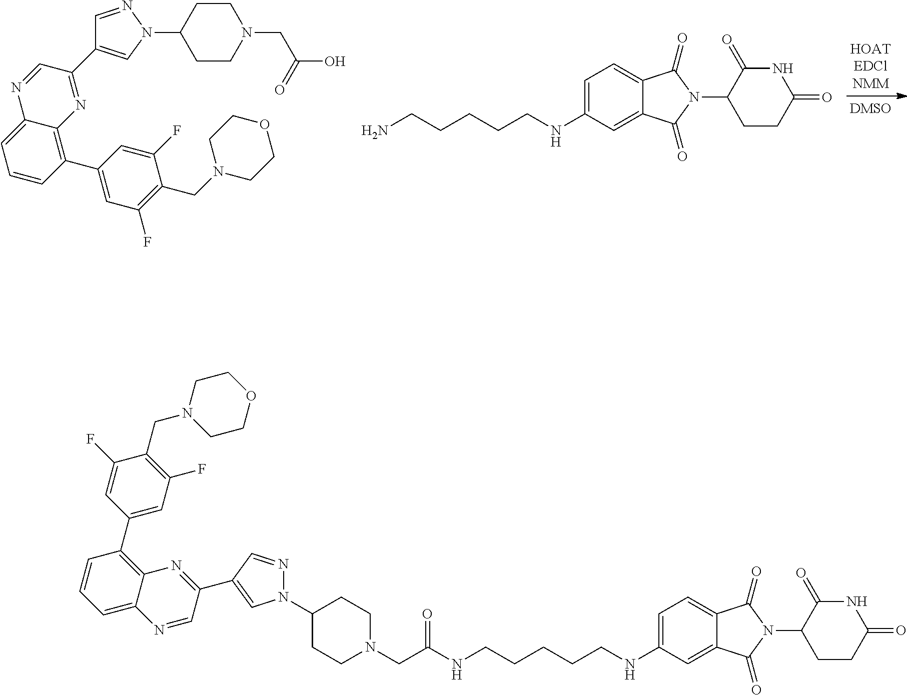 [SMILES: O=C(O)CN1CCC(n2cc(-c3cnc4cccc(-c5cc(F)c(CN6CCOCC6)c(F)c5)c4n3)cn2)CC1 ; NCCCCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCCNc1ccc2c(c1)C(=O)N(C1CCC(=O)NC1=O)C2=O]

JA-177 was synthesized following the standard procedure for preparing JA-001 (5.8 mg, 34.6% yield). MS (ESI) m/z: 889.4 [M+H]⁺.

### Example 266. 2-(4-(4-(8-(3,5-Difluoro-4-(morpholinomethyl)phenyl)quinoxalin-2-yl)-1H-pyrazol-1-yl)piperidin-1-yl)-N-(6-((2-(2,6-dioxopiperidin-3-yl)-1,3-dioxoisoindolin-5-yl)amino)hexyl)acetamide (JA-178)

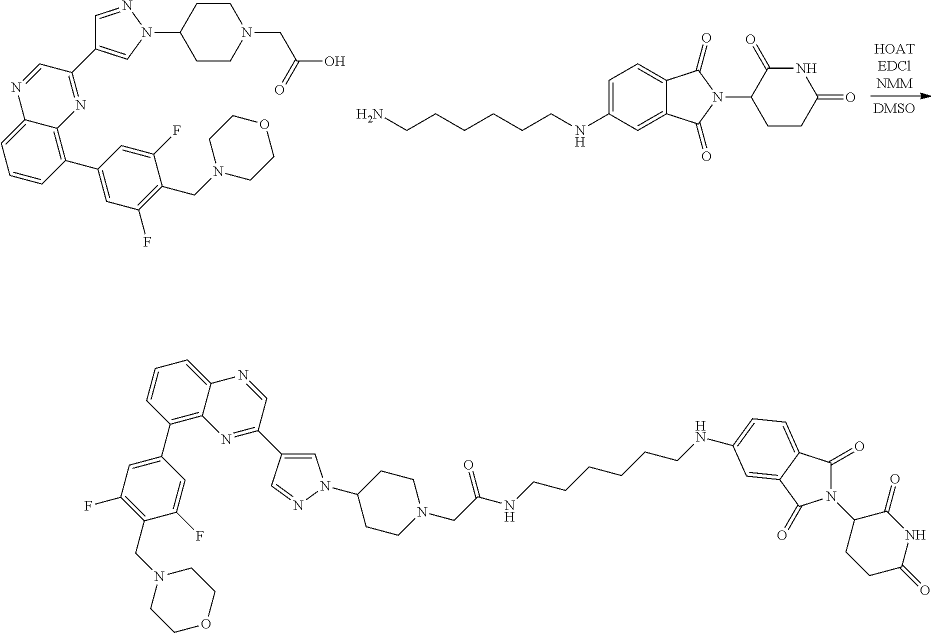 [SMILES: O=C(O)CN1CCC(n2cc(-c3cnc4cccc(-c5cc(F)c(CN6CCOCC6)c(F)c5)c4n3)cn2)CC1 ; NCCCCCCNc1ccc2c(c1)C(=O)N(C1CCC(=O)NC1=O)C2=O ; O=C(CN1CCC(n2cc(-c3cnc4cccc(-c5cc(F)c(CN6CCOCC6)c(F)c5)c4n3)cn2)CC1)NCCCCCCNc1ccc2c(c1)C(=O)N(C1CCC(=O)NC1=O)C2=O]

JA-178 was synthesized following the standard procedure for preparing JA-001 (6.1 mg, 39.5% yield). MS (ESI) m/z: 903.4 [M+H]⁺.

### Example 267. 2-(4-(4-(8-(3,5-Difluoro-4-(morpholinomethyl)phenyl)quinoxalin-2-yl)-1H-pyrazol-1-yl)piperidin-1-yl)-N-(7-((2-(2,6-dioxopiperidin-3-yl)-1,3-dioxoisoindolin-5-yl)amino)heptyl)acetamide (JA-179)

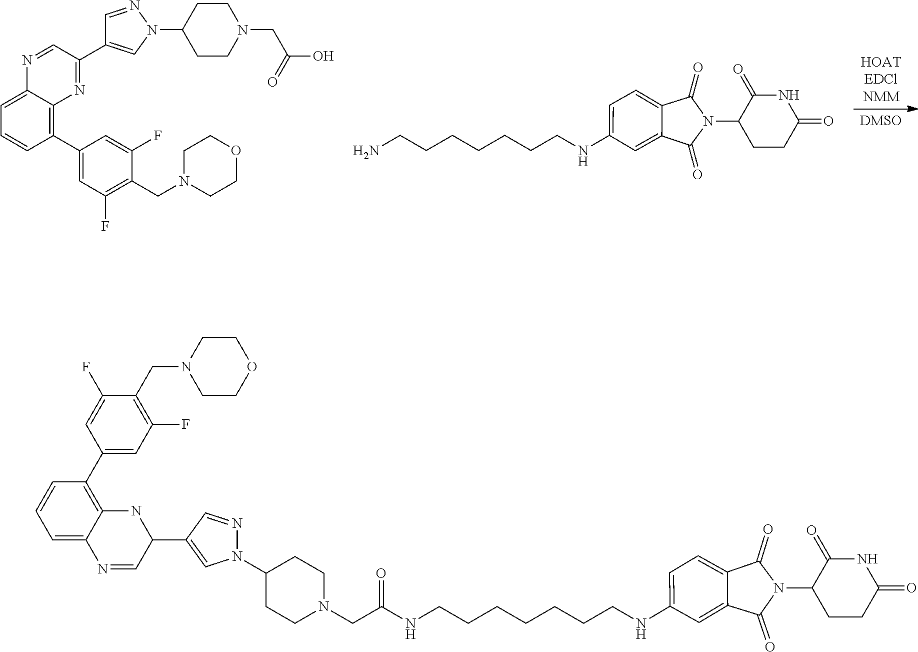

JA-179 was synthesized following the standard procedure for preparing JA-001 (5.4 mg, 35.9% yield). MS (ESI) m/z: 917.4 [M+H]⁺.

### Example 268. 2-(4-(4-(8-(3,5-Difluoro-4-(morpholinomethyl)phenyl)quinoxalin-2-yl)-1H-pyrazol-1-yl)piperidin-1-yl)-N-(8-((2-(2,6-dioxopiperidin-3-yl)-1,3-dioxoisoindolin-5-yl)amino)octyl)acetamide (JA-180)

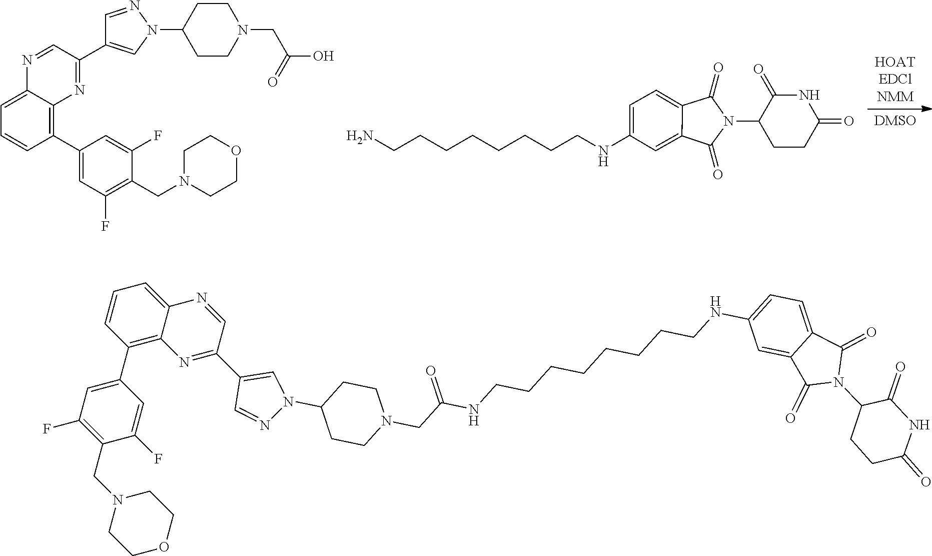

JA-180 was synthesized following the standard procedure for preparing JA-001 (4.7 mg, 32.3% yield). MS (ESI) m/z: 931.4 [M+H]⁺.

### Example 269. 5-((2-(3-(4-(4-(8-(3,5-Difluoro-4-(morpholinomethyl)phenyl)quinoxalin-2-yl)-1H-pyrazol-1-yl)piperidin-1-yl)-3-oxopropoxy)ethyl)amino)-2-(2,6-dioxopiperidin-3-yl)isoindoline-1,3-dione (JA-181)

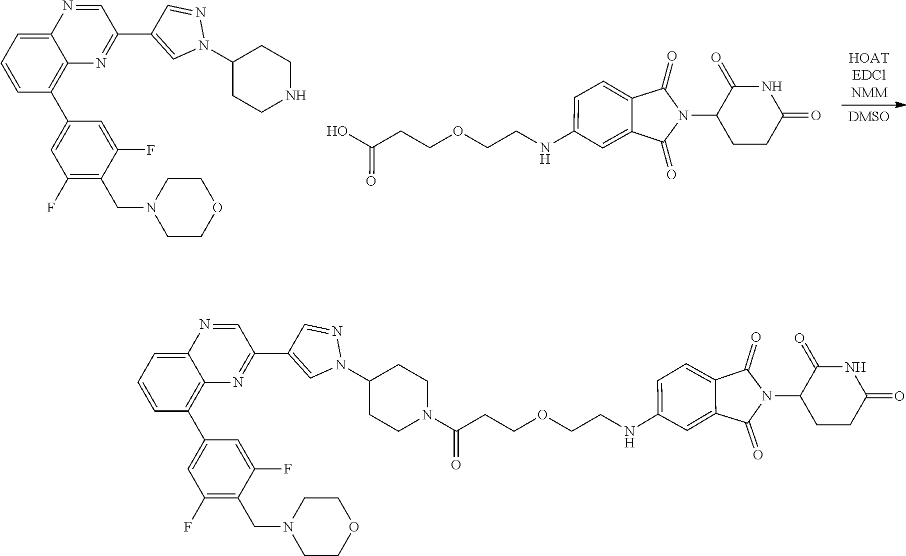 [SMILES: Fc1cc(-c2cccc3ncc(-c4cnn(C5CCNCC5)c4)nc23)cc(F)c1CN1CCOCC1 ; O=C(O)CCOCCNc1ccc2c(c1)C(=O)N(C1CCC(=O)NC1=O)C2=O ; O=C1CCC(N2C(=O)c3ccc(NCCOCCC(=O)N4CCC(n5cc(-c6cnc7cccc(-c8cc(F)c(CN9CCOCC9)c(F)c8)c7n6)cn5)CC4)cc3C2=O)C(=O)N1]

JA-181 was synthesized following the standard procedure for preparing JA-001 (5.1 mg, 33.2% yield). MS (ESI) m/z: 862.3 [M+H]⁺.

### Example 270. 5-((5-(4-(4-(8-(3,5-Difluoro-4-(morpholinomethyl)phenyl)quinoxalin-2-yl)-1H-pyrazol-1-yl)piperidin-1-yl)-5-oxopentyl)amino)-2-(2,6-dioxopiperidin-3-yl)isoindoline-1,3-dione (JA-182)

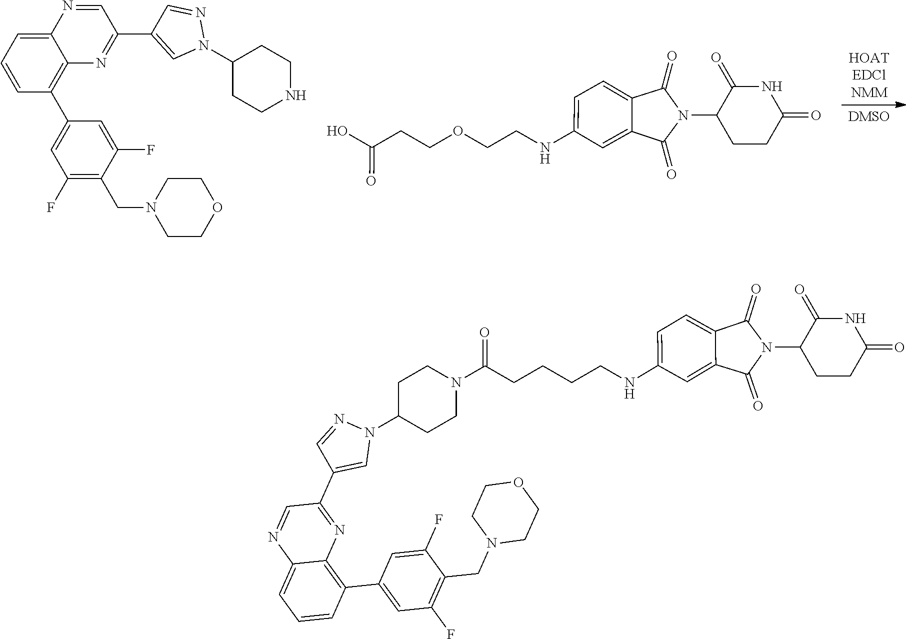 [SMILES: Fc1cc(-c2cccc3ncc(-c4cnn(C5CCNCC5)c4)nc23)cc(F)c1CN1CCOCC1 ; O=C(O)CCOCCNc1ccc2c(c1)C(=O)N(C1CCC(=O)NC1=O)C2=O ; O=C1CCC(N2C(=O)c3ccc(NCCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-182 was synthesized following the standard procedure for preparing JA-001 (5.5 mg, 35.1% yield). MS (ESI) m/z: 846.3 [M+H]⁺.

### Example 271. 5-((2-(2-(3-(4-(4-(8-(3,5-Difluoro-4-(morpholinomethyl)phenyl)quinoxalin-2-yl)-1H-pyrazol-1-yl)piperidin-1-yl)-3-oxopropoxy)ethoxy)ethyl)amino)-2-(2,6-dioxopiperidin-3-yl)isoindoline-1,3-dione (JA-183)

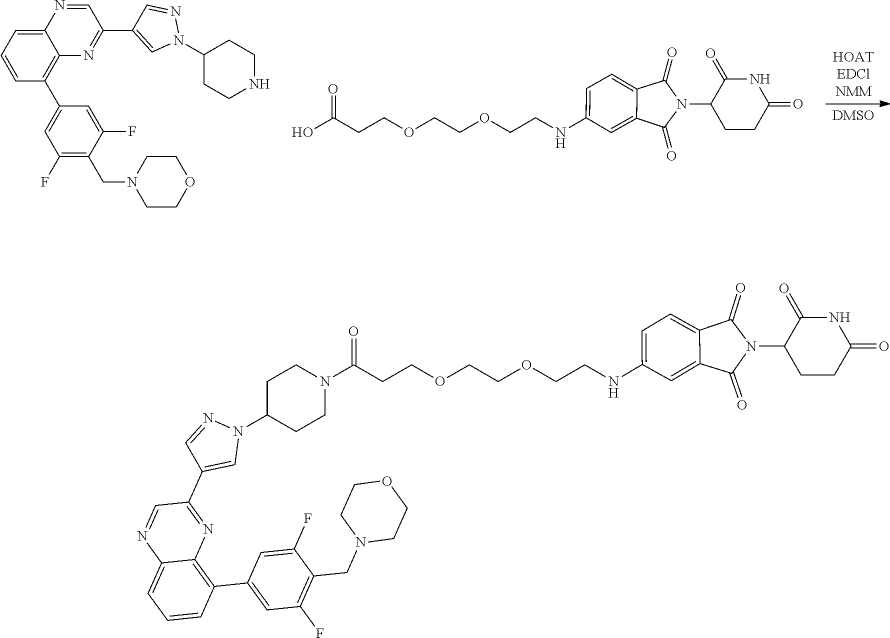 [SMILES: Fc1cc(-c2cccc3ncc(-c4cnn(C5CCNCC5)c4)nc23)cc(F)c1CN1CCOCC1 ; O=C(O)CCOCCOCCNc1ccc2c(c1)C(=O)N(C1CCC(=O)NC1=O)C2=O ; O=C1CCC(N2C(=O)c3ccc(NCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1]

JA-183 was synthesized following the standard procedure for preparing JA-001 (5.8 mg, 36.3% yield). MS (ESI) m/z: 906.4 [M+H]⁺.

### Example 272. 5-((2-(2-(2-(3-(4-(4-(8-(3,5-Difluoro-4-(morpholinomethyl)phenyl)quinoxalin-2-yl)-1H-pyrazol-1-yl)piperidin-1-yl)-3-oxopropoxy)ethoxy)ethoxy)ethyl)amino)-2-(2,6-dioxopiperidin-3-yl)isoindoline-1,3-dione (JA-184)

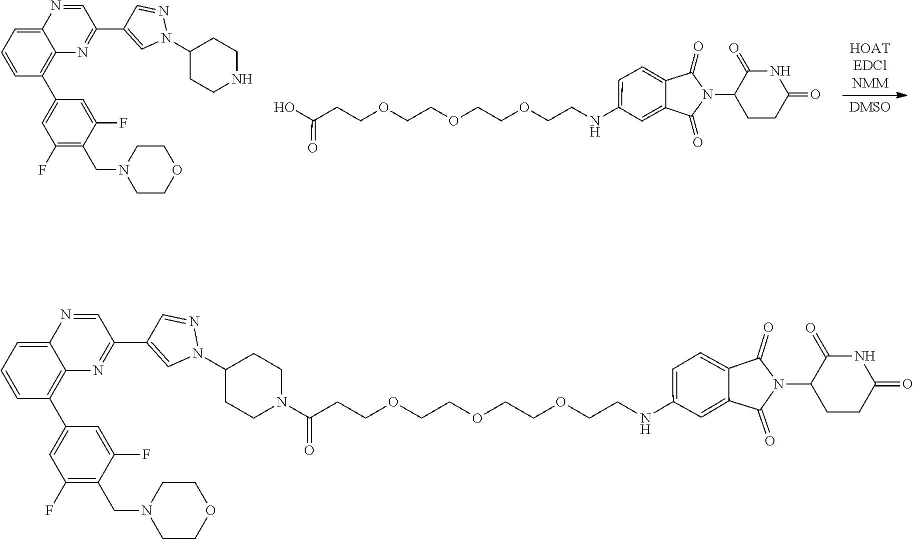 [SMILES: Fc1cc(-c2cccc3ncc(-c4cnn(C5CCNCC5)c4)nc23)cc(F)c1CN1CCOCC1 ; O=C(O)CCOCCOCCOCCNc1ccc2c(c1)C(=O)N(C1CCC(=O)NC1=O)C2=O ; O=C1CCC(N2C(=O)c3ccc(NCCOCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1]

JA-184 was synthesized following the standard procedure for preparing JA-001 (5.9 mg, 37.8% yield). MS (ESI) m/z: 950.4 [M+H]⁺.

### Example 273. 5-((15-(4-(4-(8-(3,5-Difluoro-4-(morpholinomethyl)phenyl)quinoxalin-2-yl)-1H-pyrazol-1-yl)piperidin-1-yl)-15-oxo-3,6,9,12-tetraoxapentadecyl)amino)-2-(2,6-dioxopiperidin-3-yl)isoindoline-1,3-dione (JA-185)

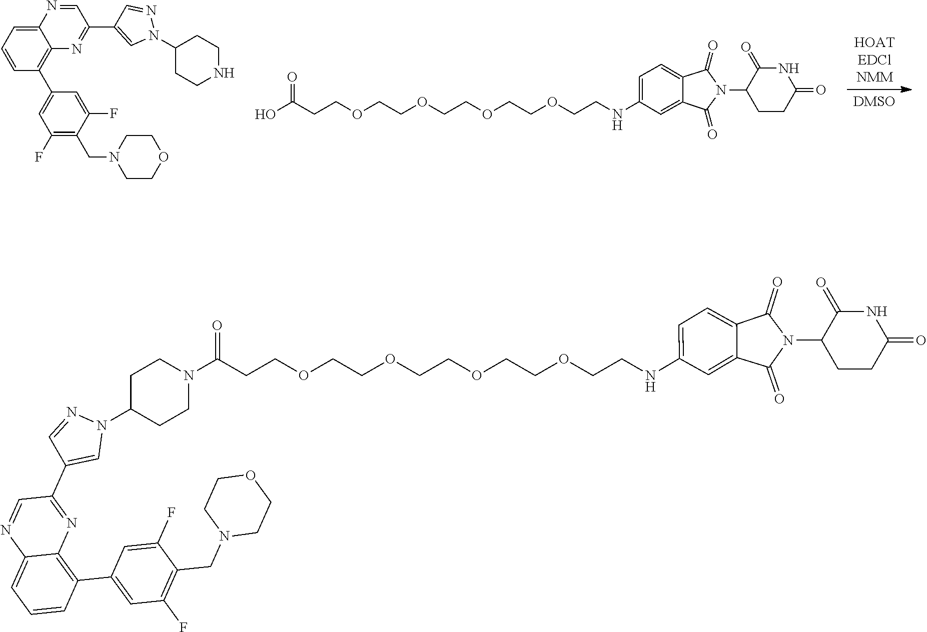 [SMILES: Fc1cc(-c2cccc3ncc(-c4cnn(C5CCNCC5)c4)nc23)cc(F)c1CN1CCOCC1 ; O=C(O)CCOCCOCCOCCOCCNc1ccc2c(c1)C(=O)N(C1CCC(=O)NC1=O)C2=O ; O=C1CCC(N2C(=O)c3ccc(NCCOCCOCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1]

JA-185 was synthesized following the standard procedure for preparing JA-001 (5.2 mg, 33.9% yield). MS (ESI) m/z: 994.4 [M+H]⁺.

### Example 274. 5-((18-(4-(4-(8-(3,5-Difluoro-4-(morpholinomethyl)phenyl)quinoxalin-2-yl)-1H-pyrazol-1-yl)piperidin-1-yl)-18-oxo-3,6,9,12,15-pentaoxaoctadecyl)amino)-2-(2,6-dioxopiperidin-3-yl)isoindoline-1,3-dione (JA-186)

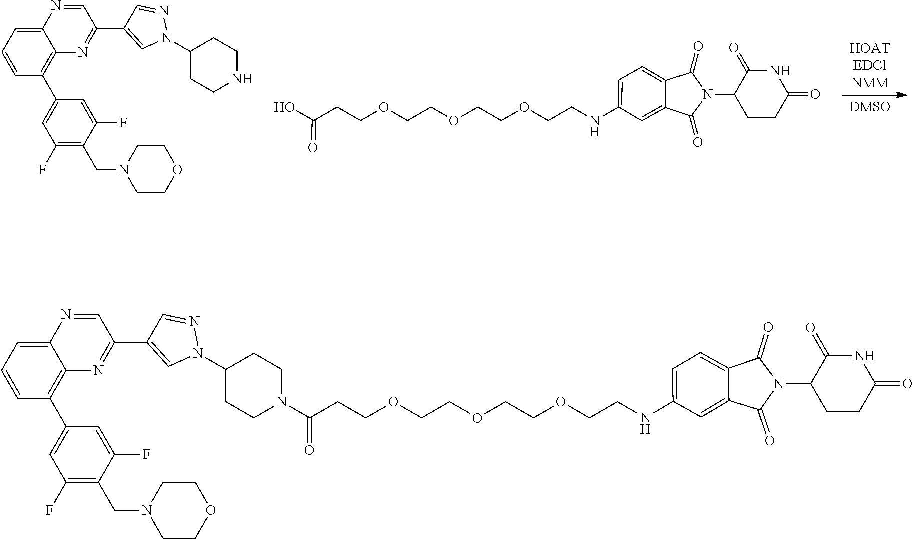 [SMILES: Fc1cc(-c2cccc3ncc(-c4cnn(C5CCNCC5)c4)nc23)cc(F)c1CN1CCOCC1 ; O=C(O)CCOCCOCCOCCNc1ccc2c(c1)C(=O)N(C1CCC(=O)NC1=O)C2=O ; O=C1CCC(N2C(=O)c3ccc(NCCOCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1]

JA-186 was synthesized following the standard procedure for preparing JA-001 (6.3 mg, 36.4% yield). MS (ESI) m/z: 1038.4 [M+H]⁺.

### Example 275. 5-((8-(4-(4-(8-(3,5-Difluoro-4-(morpholinomethyl)phenyl)quinoxalin-2-yl)-1H-pyrazol-1-yl)piperidin-1-yl)-8-oxooctyl)amino)-2-(2,6-dioxopiperidin-3-yl)isoindoline-1,3-dione-(JA-187)

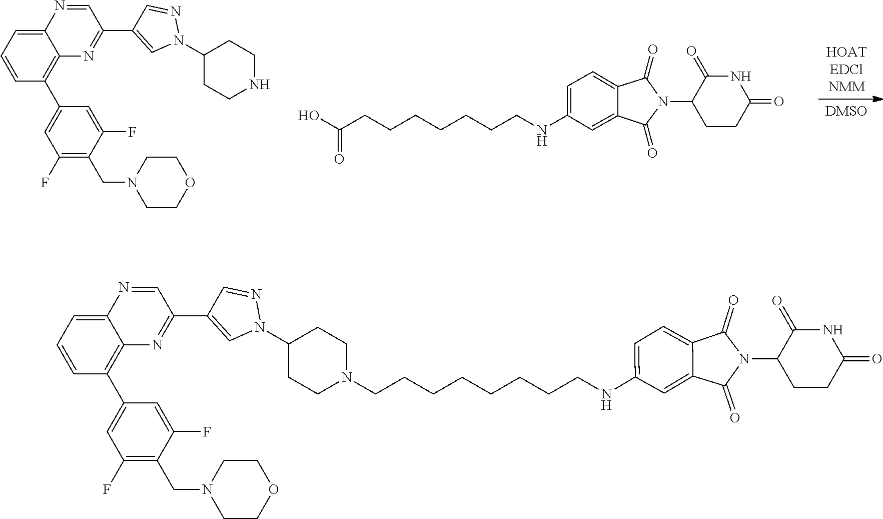 [SMILES: Fc1cc(-c2cccc3ncc(-c4cnn(C5CCNCC5)c4)nc23)cc(F)c1CN1CCOCC1 ; O=C(O)CCCCCCCNc1ccc2c(c1)C(=O)N(C1CCC(=O)NC1=O)C2=O ; O=C1CCC(N2C(=O)c3ccc(NCCCCCCCCN4CCC(n5cc(-c6cnc7cccc(-c8cc(F)c(CN9CCOCC9)c(F)c8)c7n6)cn5)CC4)cc3C2=O)C(=O)N1]

JA-187 was synthesized following the standard procedure for preparing JA-001 (7.2 mg, 44.1% yield). MS (ESI) m/z: 888.4 [M+H]⁺.

### Example 276. 5-((7-(4-(4-(8-(3,5-Difluoro-4-(morpholinomethyl)phenyl)quinoxalin-2-yl)-1H-pyrazol-1-yl)piperidin-1-yl)-7-oxoheptyl)amino)-2-(2,6-dioxopiperidin-3-yl)isoindoline-1,3-dione (JA-188)

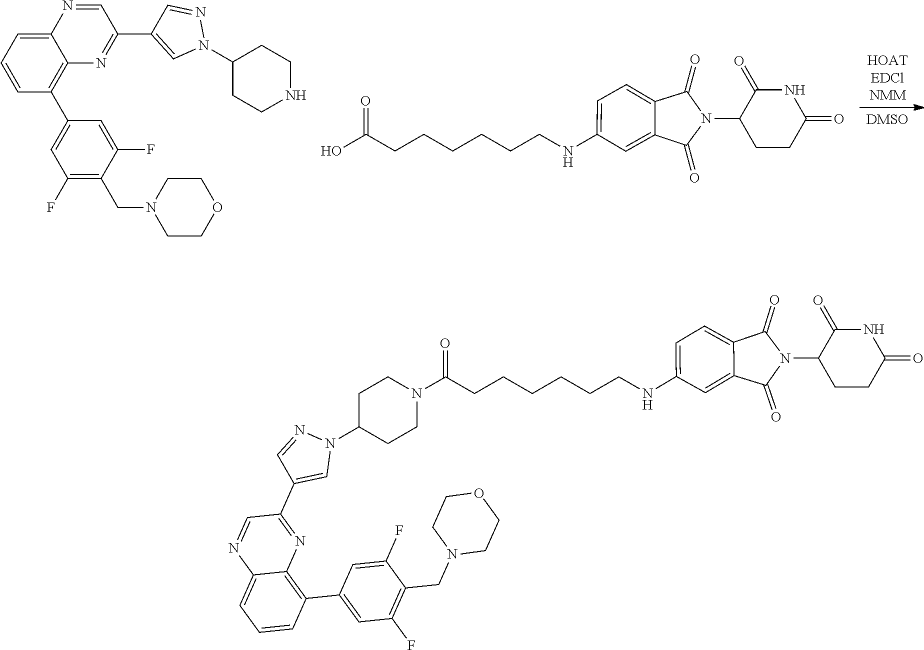 [SMILES: Fc1cc(-c2cccc3ncc(-c4cnn(C5CCNCC5)c4)nc23)cc(F)c1CN1CCOCC1 ; O=C(O)CCCCCCNc1ccc2c(c1)C(=O)N(C1CCC(=O)NC1=O)C2=O ; O=C1CCC(N2C(=O)c3ccc(NCCCCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-188 was synthesized following the standard procedure for preparing JA-001 (7.6 mg, 45.1% yield). MS (ESI) m/z: 874.4 [M+H]⁺.

### Example 277. 5-((6-(4-(4-(8-(3,5-Difluoro-4-(morpholinomethyl)phenyl)quinoxalin-2-yl)-1H-pyrazol-1-yl)piperidin-1-yl)-6-oxohexyl)amino)-2-(2,6-dioxopiperidin-3-yl)isoindoline-1,3-dione (JA-189)

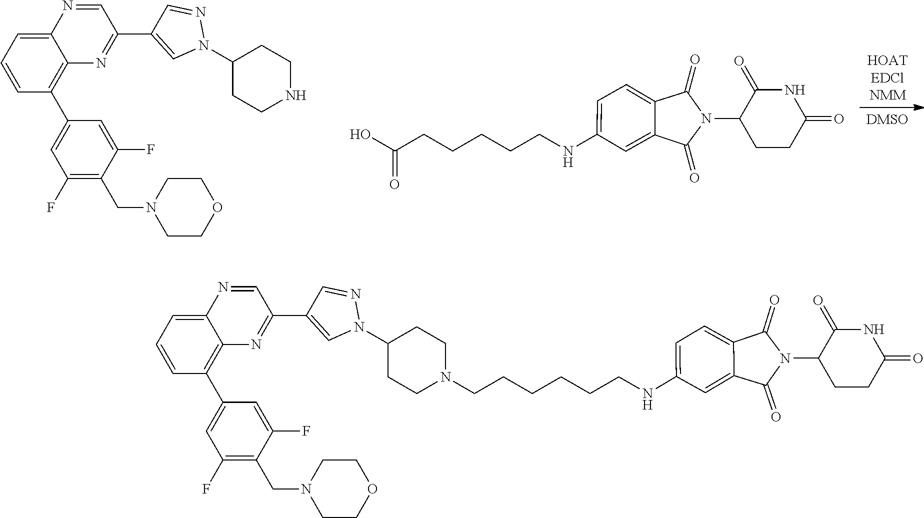 [SMILES: Fc1cc(-c2cccc3ncc(-c4cnn(C5CCNCC5)c4)nc23)cc(F)c1CN1CCOCC1 ; O=C(O)CCCCCNc1ccc2c(c1)C(=O)N(C1CCC(=O)NC1=O)C2=O ; O=C1CCC(N2C(=O)c3ccc(NCCCCCCN4CCC(n5cc(-c6cnc7cccc(-c8cc(F)c(CN9CCOCC9)c(F)c8)c7n6)cn5)CC4)cc3C2=O)C(=O)N1]

JA-189 was synthesized following the standard procedure for preparing JA-001 (6.3 mg, 38.1% yield). MS (ESI) m/z: 860.4 [M+H]⁺.

### Example 278. 5-((3-(4-(4-(8-(3,5-Difluoro-4-(morpholinomethyl)phenyl)quinoxalin-2-yl)-1H-pyrazol-1-yl)piperidin-1-yl)-3-oxopropyl)amino)-2-(2,6-dioxopiperidin-3-yl)isoindoline-1,3-dione (JA-190)

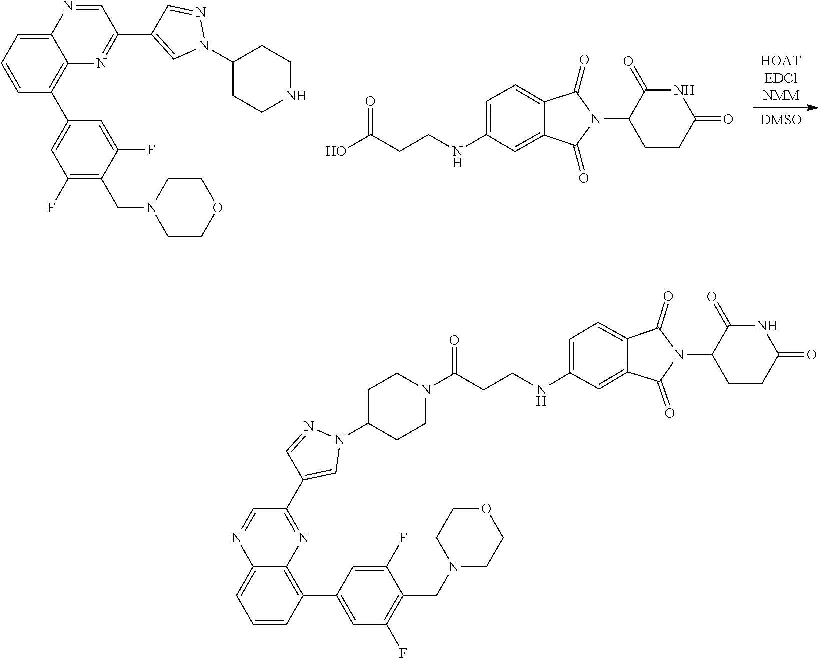 [SMILES: Fc1cc(-c2cccc3ncc(-c4cnn(C5CCNCC5)c4)nc23)cc(F)c1CN1CCOCC1 ; O=C(O)CCNc1ccc2c(c1)C(=O)N(C1CCC(=O)NC1=O)C2=O ; O=C1CCC(N2C(=O)c3ccc(NCCC(=O)N4CCC(n5cc(-c6cnc7cccc(-c8cc(F)c(CN9CCOCC9)c(F)c8)c7n6)cn5)CC4)cc3C2=O)C(=O)N1]

JA-190 was synthesized following the standard procedure for preparing JA-001 (5.7 mg, 40.1% yield). MS (ESI) m/z: 818.3 [M+H]⁺.

### Example 279. 5-((2-(4-(4-(8-(3,5-Difluoro-4-(morpholinomethyl)phenyl)quinoxalin-2-yl)-1H-pyrazol-1-yl)piperidin-1-yl)-2-oxoethyl)amino)-2-(2,6-dioxopiperidin-3-ylisoindoline-13-dione (JA-191)

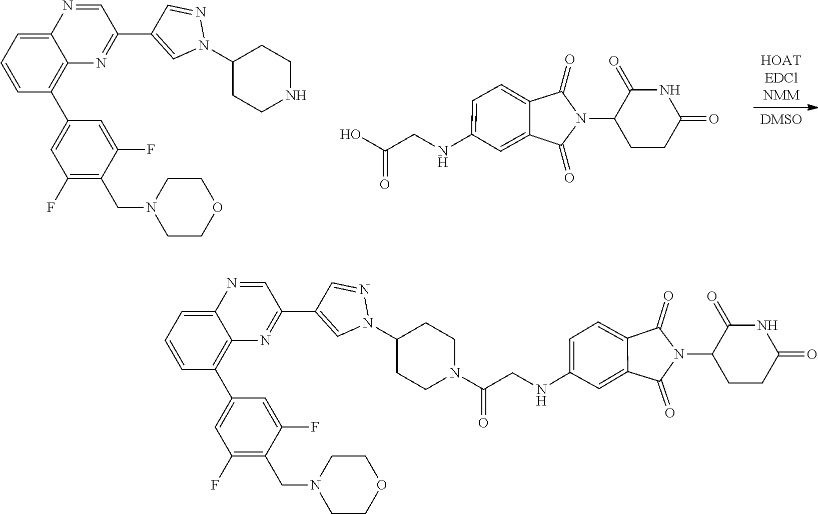 [SMILES: Fc1cc(-c2cccc3ncc(-c4cnn(C5CCNCC5)c4)nc23)cc(F)c1CN1CCOCC1 ; O=C(O)CNc1ccc2c(c1)C(=O)N(C1CCC(=O)NC1=O)C2=O ; O=C1CCC(N2C(=O)c3ccc(NCC(=O)N4CCC(n5cc(-c6cnc7cccc(-c8cc(F)c(CN9CCOCC9)c(F)c8)c7n6)cn5)CC4)cc3C2=O)C(=O)N1]

JA-191 was synthesized following the standard procedure for preparing JA-001 (5.7 mg, 34.9% yield). MS (ESI) m/z: 804.3 [M+H]⁺.

### Example 280. 5-((4-(4-(4-(8-(3,5-Difluoro-4-(morpholinomethyl)phenyl)quinoxalin-2-yl)-1H-pyrazol-1-yl)piperidin-1-yl)-4-oxobutyl)amino)-2-(2,6-dioxopiperidin-3-yl)isoindoline-1,3-dione (JA-192)

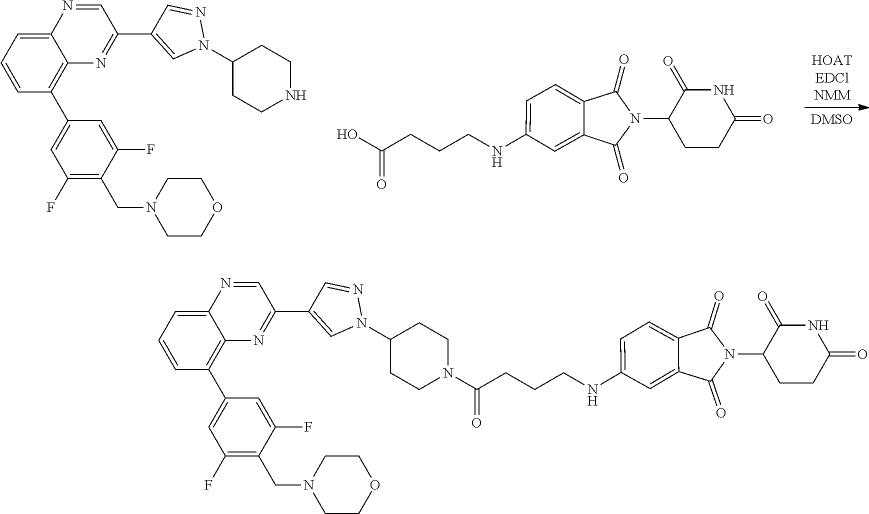 [SMILES: Fc1cc(-c2cccc3ncc(-c4cnn(C5CCNCC5)c4)nc23)cc(F)c1CN1CCOCC1 ; O=C(O)CCCNc1ccc2c(c1)C(=O)N(C1CCC(=O)NC1=O)C2=O ; O=C1CCC(N2C(=O)c3ccc(NCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-192 was synthesized following the standard procedure for preparing JA-001 (6.3 mg, 37.9% yield). MS (ESI) m/z: 832.3 [M+H]⁺.

### Example 281. 2-(2,6-Dioxopiperidin-3-yl)-5-((18-(4-(4-((5-(4-(methylsulfonyl)phenyl)-[1,2,4]triazolo[1,5-a]pyridin-2-yl)amino)phenyl)piperazin-1-yl)-18-oxo-3,6,9,12,15-pentaoxaoctadecyl)amino)isoindoline-1,3-dione (JA-193)

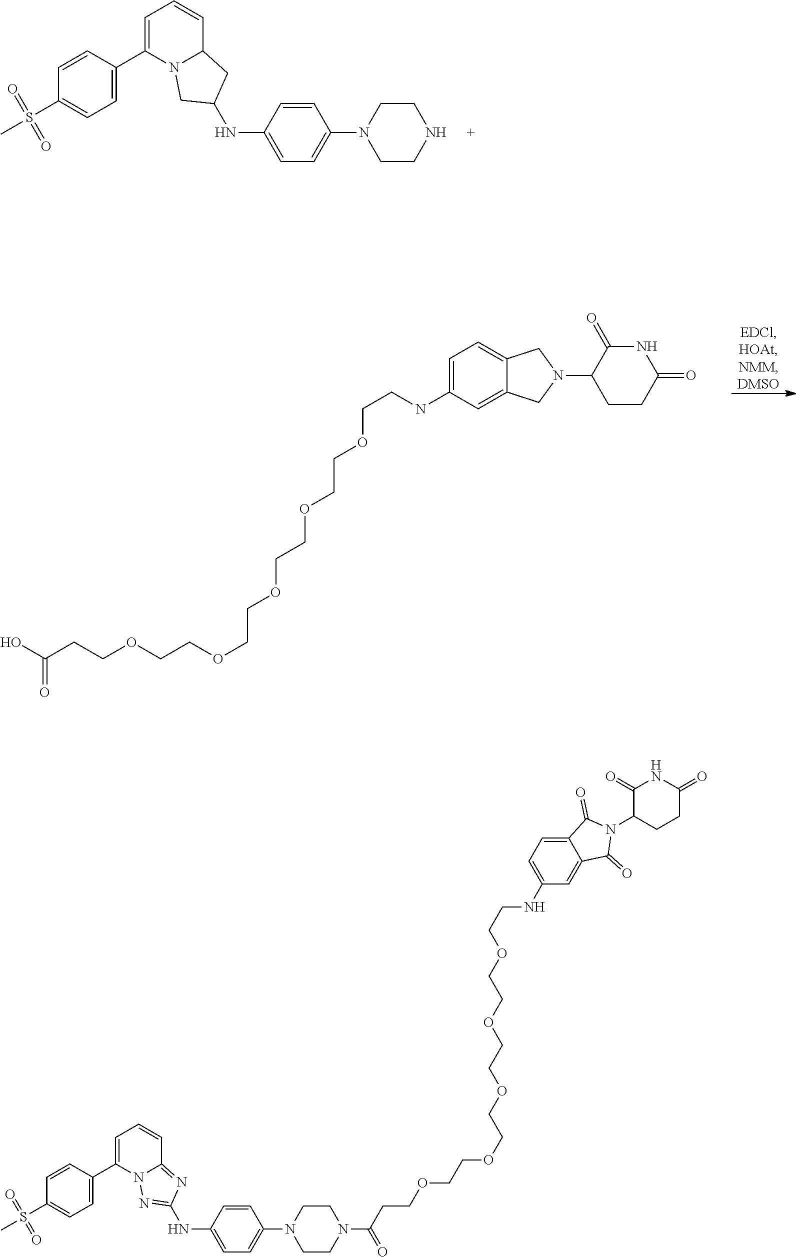

JA-193 was synthesized following the standard procedure for preparing JA-010 (6.9 mg, 56% yield). MS (ESI) m/z: 996.4 [M+H]⁺.

### Example 282. 2-(2,6-Dioxopiperidin-3-yl)-5-((2-(4-(4-((5-(4-(methylsulfonyl)phenyl)-[1,2,4]triazolo[1,5-a]pyridin-2-yl)amino)phenyl)piperazin-1-yl)-2-oxoethyl)amino)isoindoline-1,3-dione (JA-194)

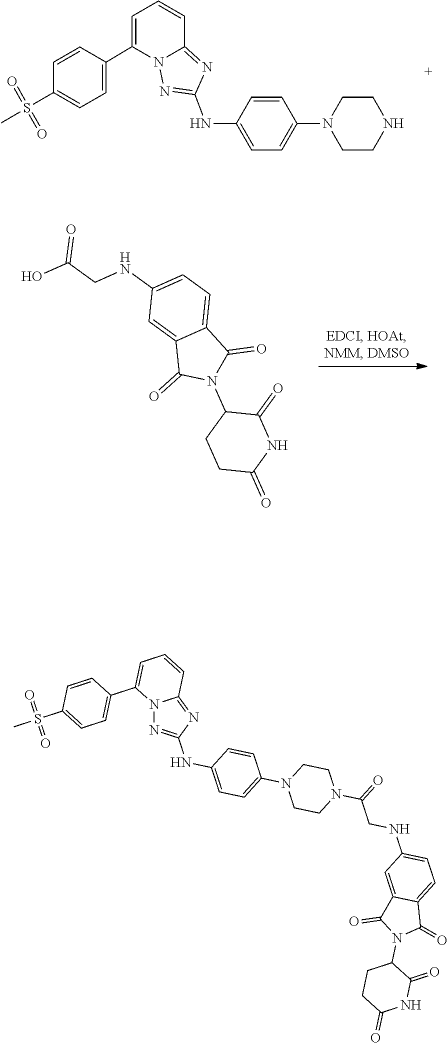 [SMILES: CS(=O)(=O)c1ccc(-c2cccc3nc(Nc4ccc(N5CCNCC5)cc4)nn23)cc1 ; O=C(O)CNc1ccc2c(c1)C(=O)N(C1CCC(=O)NC1=O)C2=O ; CS(=O)(=O)c1ccc(-c2cccc3nc(Nc4ccc(N5CCN(C(=O)CNc6ccc7c(c6)C(=O)N(C6CCC(=O)NC6=O)C7=O)CC5)cc4)nn23)cc1]

JA-194 was synthesized following the standard procedure for preparing JA-010 (7.2 mg, 60% yield). MS (ESI) m/z: 762.2 [M+H]⁺.

### Example 283. 2-(2,6-Dioxopiperidin-3-yl)-5-((2-(3-(4-(4-((5-(4-(methylsulfonyl)phenyl)-[1,2,4]triazolo[1,5-a]pyridin-2-yl)amino)phenyl)piperazin-1-yl)-3-oxopropoxy)ethyl)amino)isoindoline-1,3-dione (JA-195)

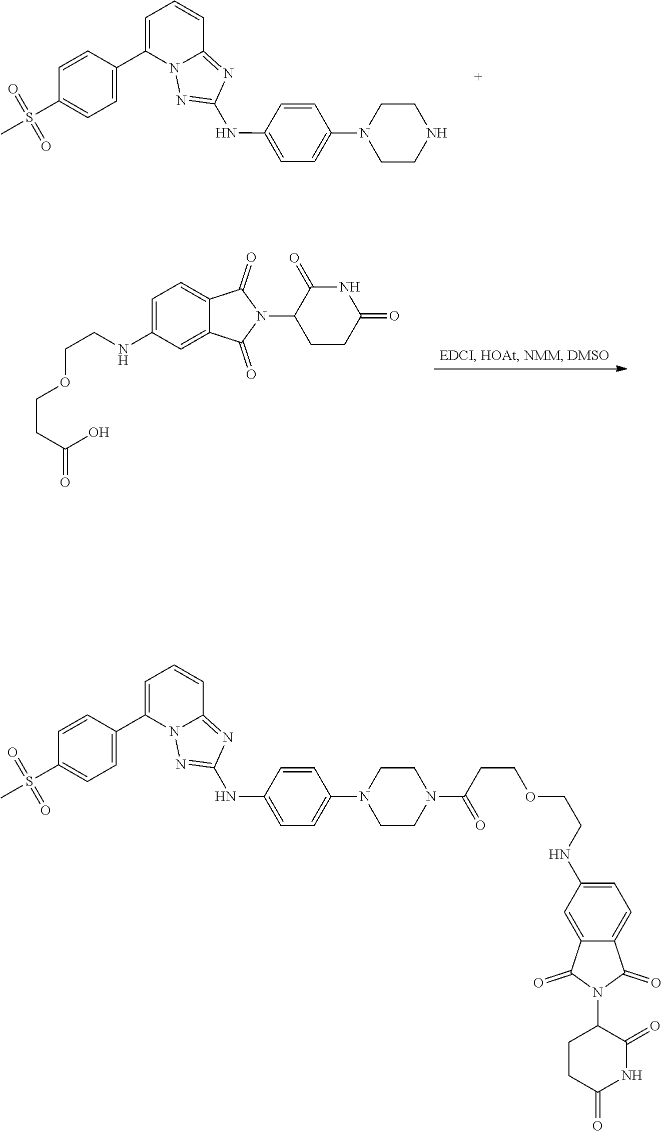 [SMILES: CS(=O)(=O)c1ccc(-c2cccc3nc(Nc4ccc(N5CCNCC5)cc4)nn23)cc1 ; O=C(O)CCOCCNc1ccc2c(c1)C(=O)N(C1CCC(=O)NC1=O)C2=O ; CS(=O)(=O)c1ccc(-c2cccc3nc(Nc4ccc(N5CCN(C(=O)CCOCCNc6ccc7c(c6)C(=O)N(C6CCC(=O)NC6=O)C7=O)CC5)cc4)nn23)cc1]

JA-195 was synthesized following the standard procedure for preparing JA-010 (7.0 mg, 61% yield). MS (ESI) m/z: 820.3 [M+H]⁺.

### Example 284. 2-(2,6-Dioxopiperidin-3-yl)-5-((5-(4-(4-((5-(4-(methylsulfonyl)phenyl)-[1,2,4]triazolo[1,5-a]pyridin-2-yl)amino)phenyl)piperazin-1-yl)-5-oxopentyl)amino)isoindoline-1,3-dione (JA-196)

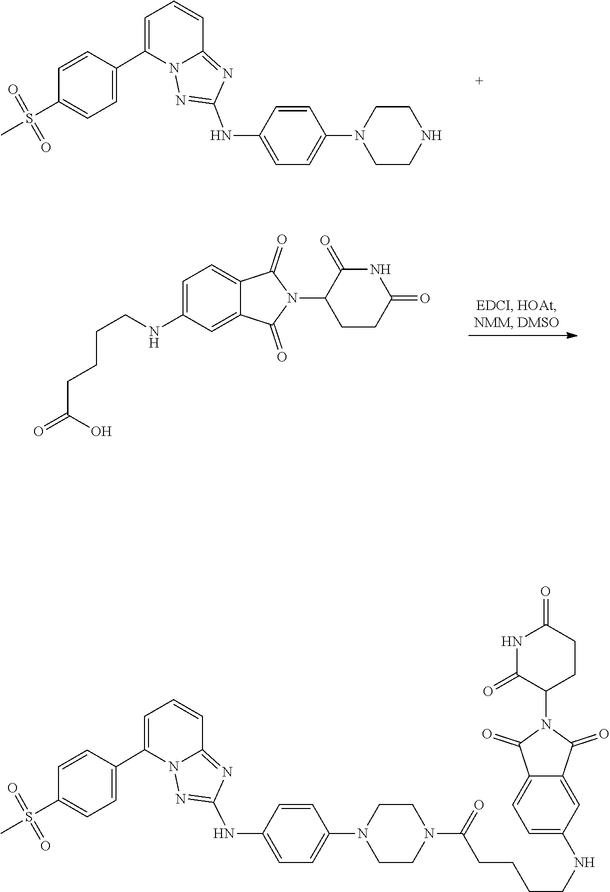 [SMILES: CS(=O)(=O)c1ccc(-c2cccc3nc(Nc4ccc(N5CCNCC5)cc4)nn23)cc1 ; O=C(O)CCCCNc1ccc2c(c1)C(=O)N(C1CCC(=O)NC1=O)C2=O ; CS(=O)(=O)c1ccc(-c2cccc3nc(Nc4ccc(N5CCN(C(=O)CCCCNc6ccc7c(c6)C(=O)N(C6CCC(=O)NC6=O)C7=O)CC5)cc4)nn23)cc1]

JA-196 was synthesized following the standard procedure for preparing JA-010 (7.2 mg, 62% yield). MS (ESI) m/z: 804.3 [M+H]⁺.

### Example 285. 2-(2,6-Dioxopiperidin-3-yl)-5-((15-(4-(4-((5-(4-(methylsulfonyl)phenyl)-[1,2,4]triazolo[1,5-a]pyridin-2-yl)amino)phenyl)piperazin-1-yl)-15-oxo-3,6,9,12-tetraoxapentadecyl)amino)isoindoline-1,3-dione (JA-197)

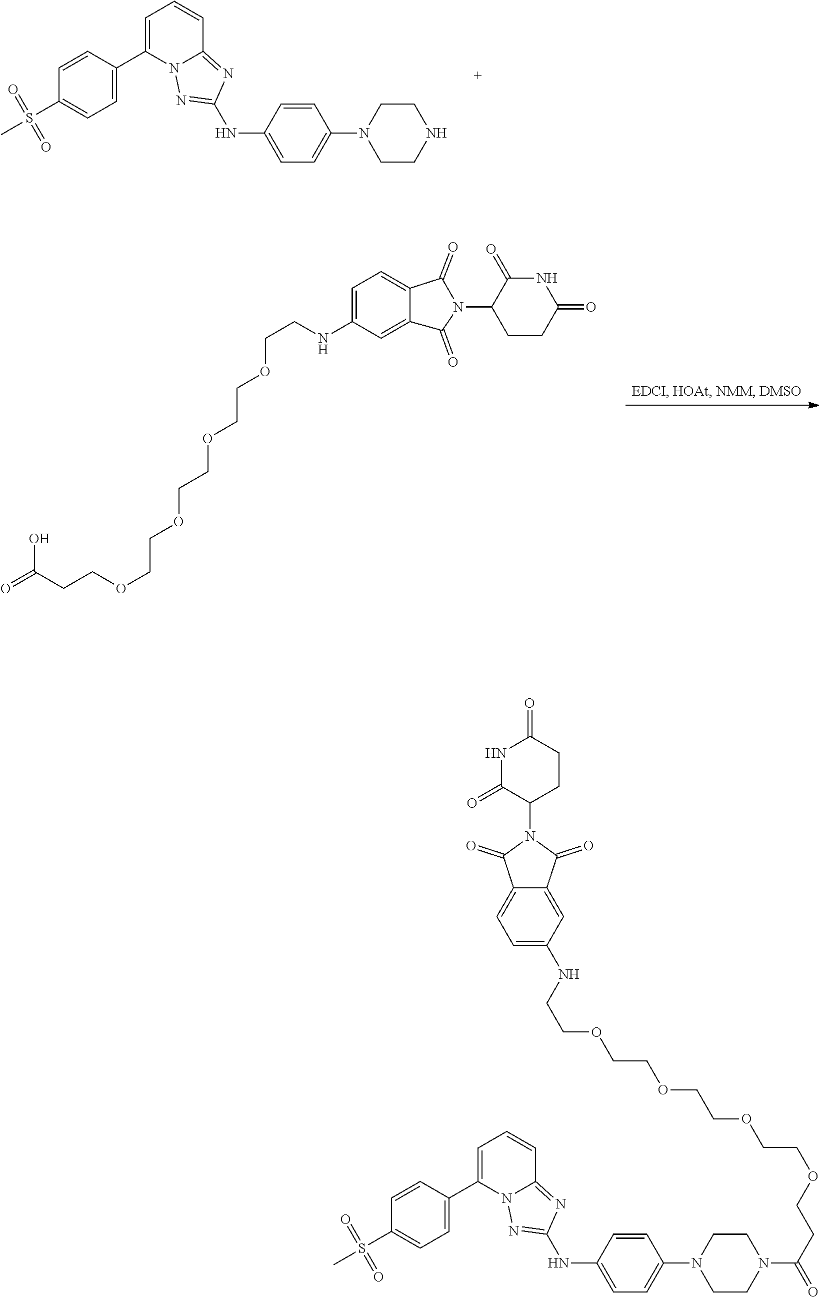 [SMILES: CS(=O)(=O)c1ccc(-c2cccc3nc(Nc4ccc(N5CCNCC5)cc4)nn23)cc1 ; O=C(O)CCOCCOCCOCCOCCNc1ccc2c(c1)C(=O)N(C1CCC(=O)NC1=O)C2=O ; CS(=O)(=O)c1ccc(-c2cccc3nc(Nc4ccc(N5CCN(C(=O)CCOCCOCCOCCOCCNc6ccc7c(c6)C(=O)N(C6CCC(=O)NC6=O)C7=O)CC5)cc4)nn23)cc1]

JA-197 was synthesized following the standard procedure for preparing JA-010 (7.6 mg, 65% yield). MS (ESI) m/z: 952.4 [M+H]⁺.

### Example 286. 2-(2,6-Dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)isoindoline-1,3-dione (JA-198)

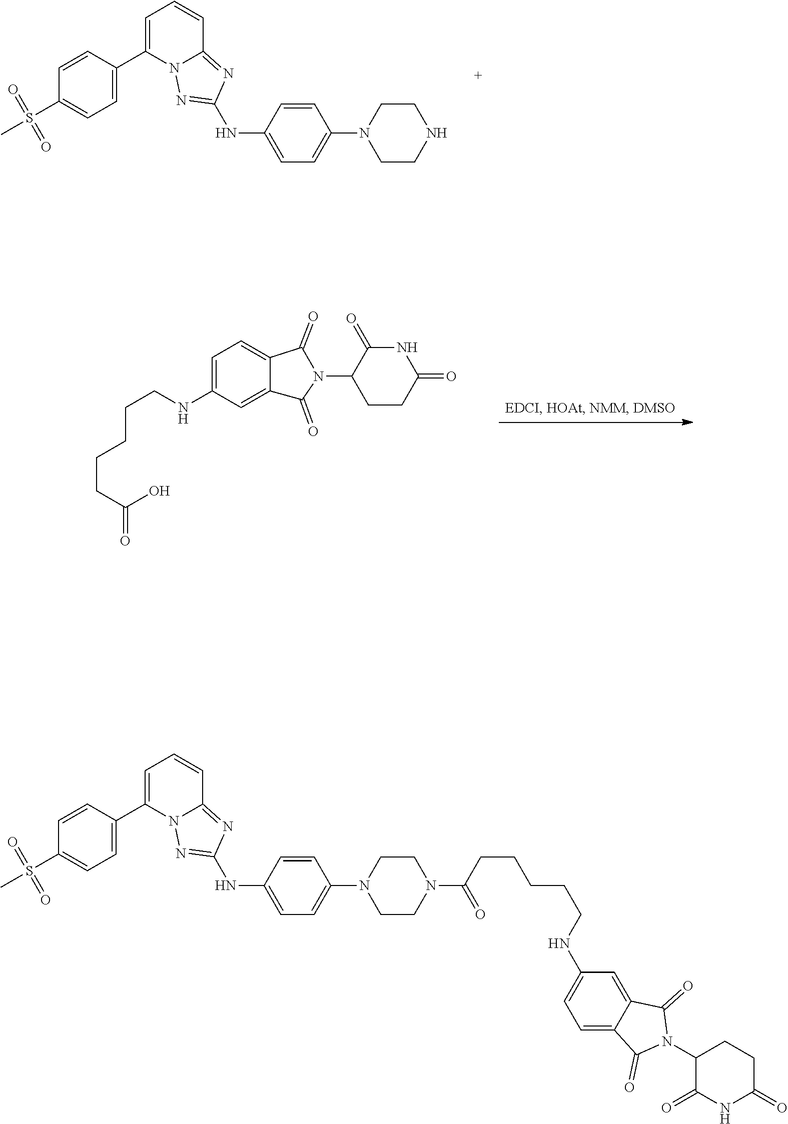

JA-198 was synthesized following the standard procedure for preparing JA-010 (7.1 mg, 64% yield). MS (ESI) m/z: 818.3 [M+H]⁺.

### Example 287. 2-(2,6-Dioxopiperidin-3-yl)-5-((7-(4-(4-((5-(4-(methylsulfonyl)phenyl)-[1,2,4]triazolo[1,5-a]pyridin-2-yl)amino)phenyl)piperazin-1-yl)-7-oxoheptyl)amino)isoindoline-1,3-dione (JA-199)

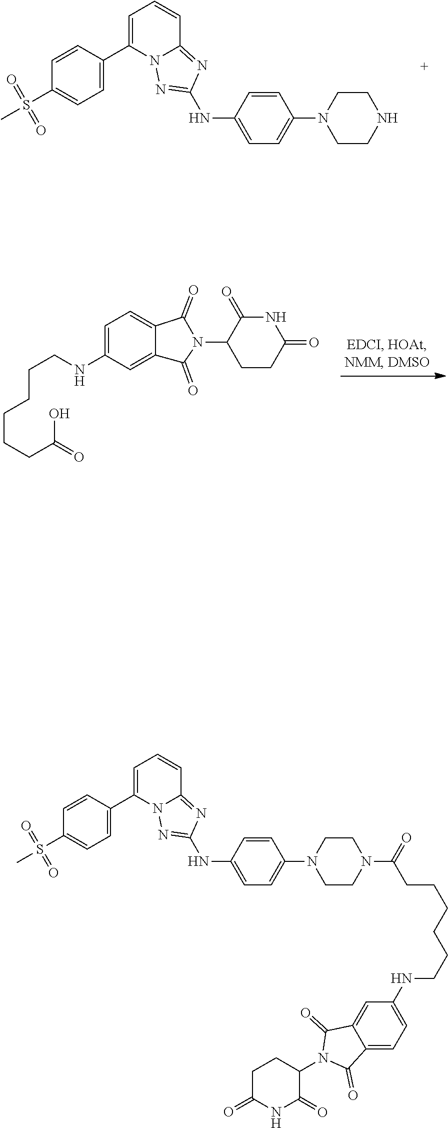

JA-199 was synthesized following the standard procedure for preparing JA-010 (7.1 mg, 64% yield). MS (ESI) m/z: 832.3 [M+H]⁺.

### Example 288. 2-(2,6-Dioxopiperidin-3-yl)-5-((2-(2-(2-(3-(4-(4-((5-(4-(methylsulfonyl)phenyl)-[1,2,4]triazolo[1,5-a]pyridin-2-yl)amino)phenyl)piperazin-1-yl)-3-oxopropoxy)ethoxy)ethoxy)ethyl)amino)isoindoline-1,3-dione (JA-200)

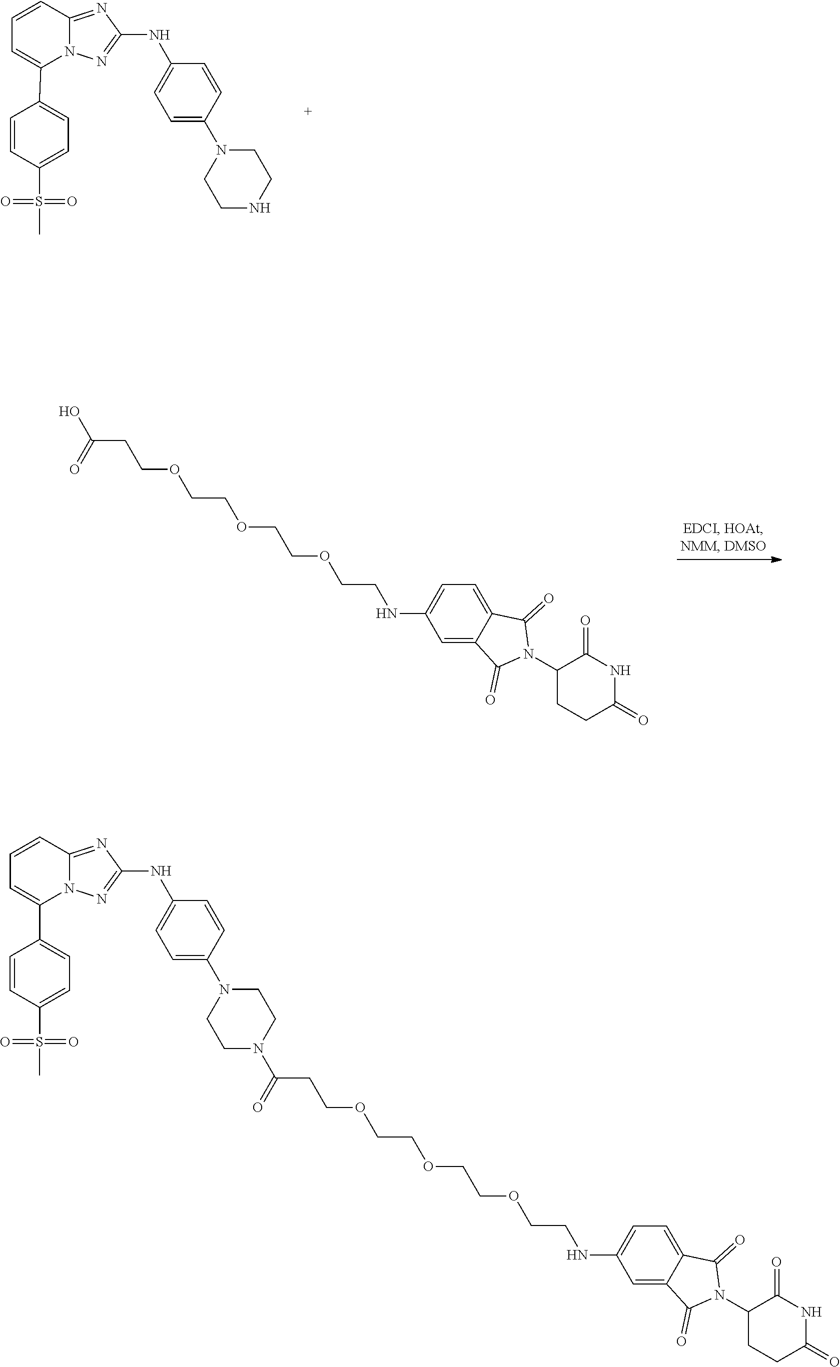 [SMILES: CS(=O)(=O)c1ccc(-c2cccc3nc(Nc4ccc(N5CCNCC5)cc4)nn23)cc1 ; O=C(O)CCOCCOCCOCCNc1ccc2c(c1)C(=O)N(C1CCC(=O)NC1=O)C2=O ; CS(=O)(=O)c1ccc(-c2cccc3nc(Nc4ccc(N5CCN(C(=O)CCOCCOCCOCCNc6ccc7c(c6)C(=O)N(C6CCC(=O)NC6=O)C7=O)CC5)cc4)nn23)cc1]

JA-200 was synthesized following the standard procedure for preparing JA-010 (8.3 mg, 65% yield). MS (ESI) m/z: 908.4 [M+H]⁺.

### Example 289. 2-(2,6-Dioxopiperidin-3-yl)-5-((2-(2-(3-(4-(4-((5-(4-(methylsulfonyl)phenyl)-[1,2,4]triazolo[1,5-a]pyridin-2-yl)amino)phenyl)piperazin-1-yl)-3-oxopropoxy ethoxy)ethyl)amino)isoindoline-1,3-dione (JA-201)

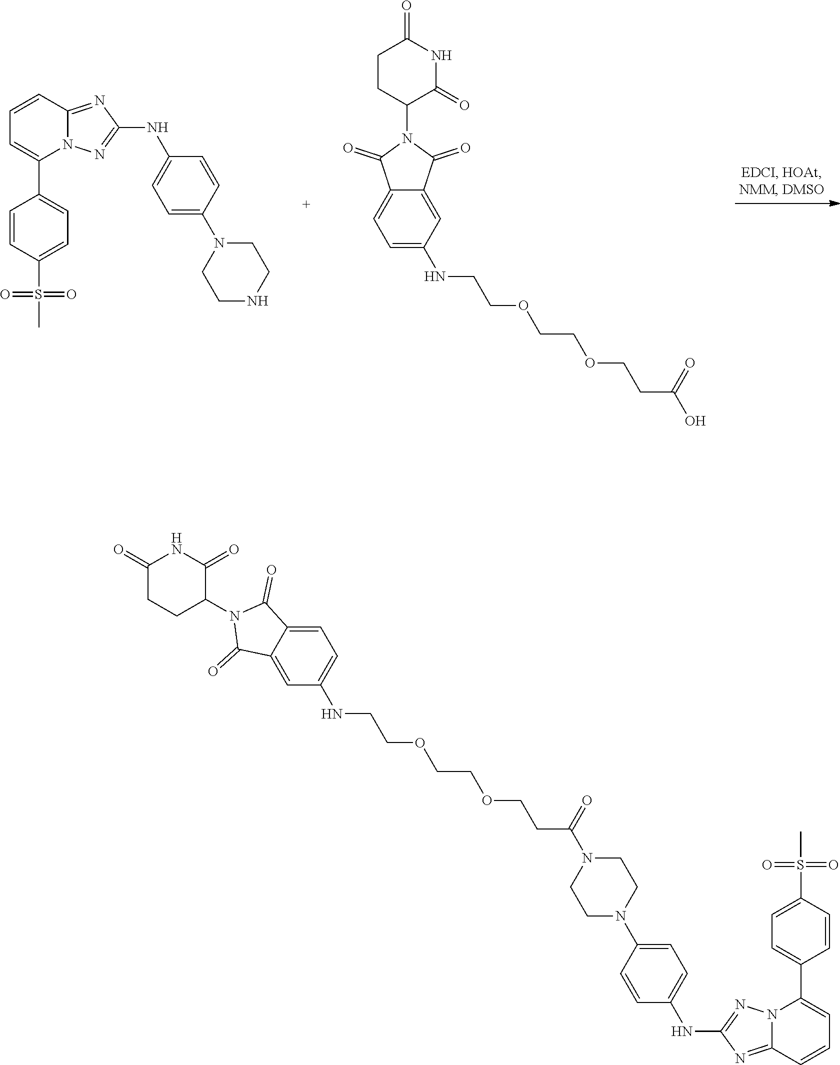 [SMILES: CS(=O)(=O)c1ccc(-c2cccc3nc(Nc4ccc(N5CCNCC5)cc4)nn23)cc1 ; O=C(O)CCOCCOCCNc1ccc2c(c1)C(=O)N(C1CCC(=O)NC1=O)C2=O ; CS(=O)(=O)c1ccc(-c2cccc3nc(Nc4ccc(N5CCN(C(=O)CCOCCOCCNc6ccc7c(c6)C(=O)N(C6CCC(=O)NC6=O)C7=O)CC5)cc4)nn23)cc1]

JA-201 was synthesized following the standard procedure for preparing JA-010 (7.3 mg, 66% yield). MS (ESI) m/z: 864.3 [M+H]⁺.

### Example 290. 2-(2,6-Dioxopiperidin-3-yl)-5-((3-(4-(4-((5-(4-(methylsulfonyl)phenyl)-[1,2,4]triazolo[1,5-a]pyridin-2-yl)amino)phenyl)piperazin-1-yl)-3-oxopropyl)amino)isoindoline-1,3-dione (JA-202)

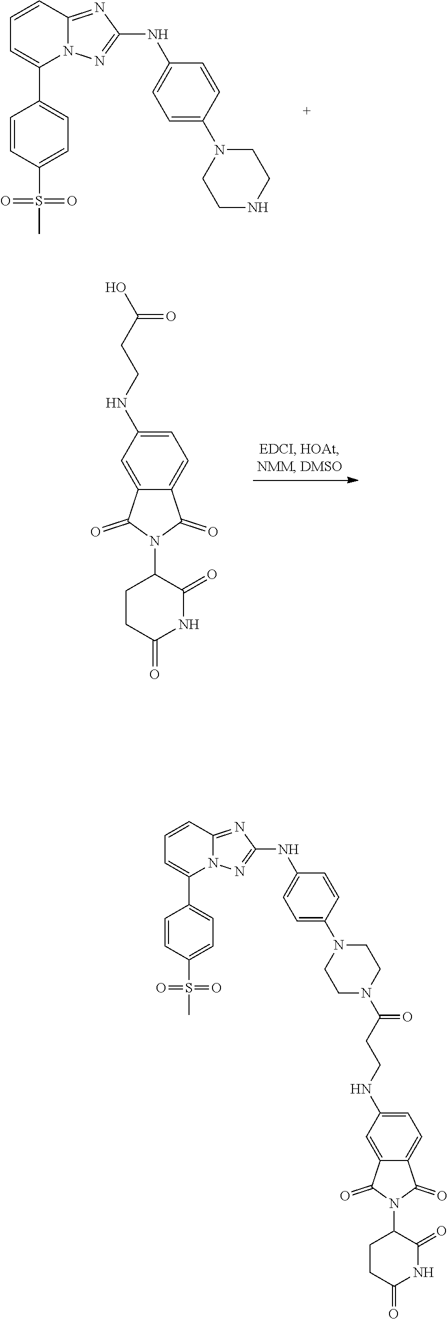 [SMILES: CS(=O)(=O)c1ccc(-c2cccc3nc(Nc4ccc(N5CCNCC5)cc4)nn23)cc1 ; O=C(O)CCNc1ccc2c(c1)C(=O)N(C1CCC(=O)NC1=O)C2=O ; CS(=O)(=O)c1ccc(-c2cccc3nc(Nc4ccc(N5CCN(C(=O)CCNc6ccc7c(c6)C(=O)N(C6CCC(=O)NC6=O)C7=O)CC5)cc4)nn23)cc1]

JA-202 was synthesized following the standard procedure for preparing JA-010 (7.6 mg, 68% yield). MS (ESI) m/z: 776.3 [M+H]⁺.

### Example 291. 2-(2,6-Dioxopiperidin-3-yl)-5-((8-(4-(4-((5-(4-(methylsulfonyl)phenyl)-[1,2,4]triazolo[1,5-a]pyridin-2-yl)amino)phenyl)piperazin-1-yl)-8-oxooctyl)amino)isoindoline-1,3-dione (JA-203)

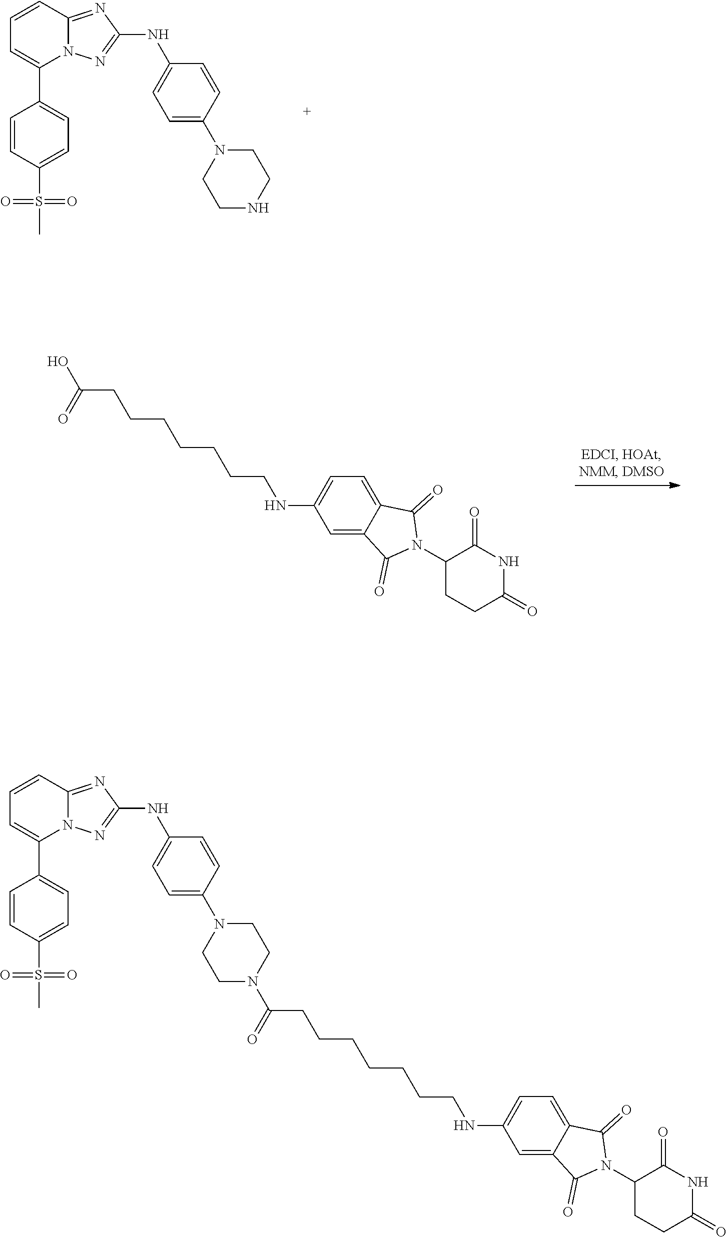 [SMILES: CS(=O)(=O)c1ccc(-c2cccc3nc(Nc4ccc(N5CCNCC5)cc4)nn23)cc1 ; O=C(O)CCCCCCCNc1ccc2c(c1)C(=O)N(C1CCC(=O)NC1=O)C2=O ; CS(=O)(=O)c1ccc(-c2cccc3nc(Nc4ccc(N5CCN(C(=O)CCCCCCCNc6ccc7c(c6)C(=O)N(C6CCC(=O)NC6=O)C7=O)CC5)cc4)nn23)cc1]

JA-203 was synthesized following the standard procedure for preparing JA-010 (7.1 mg, 65% yield). MS (ESI) m/z: 846.4 [M+H]⁺.

### Example 292. N-(5-((2-(2,6-Dioxopiperidin-3-yl)-1,3-dioxoisoindolin-5-yl)amino)pentyl)-2-(4-(4-((5-(4-(methylsulfonyl)phenyl)-[1,2,4]triazolo[1,5-a]pyridin-2-yl)amino)phenyl)piperazin-1-yl)acetamide (JA-204)

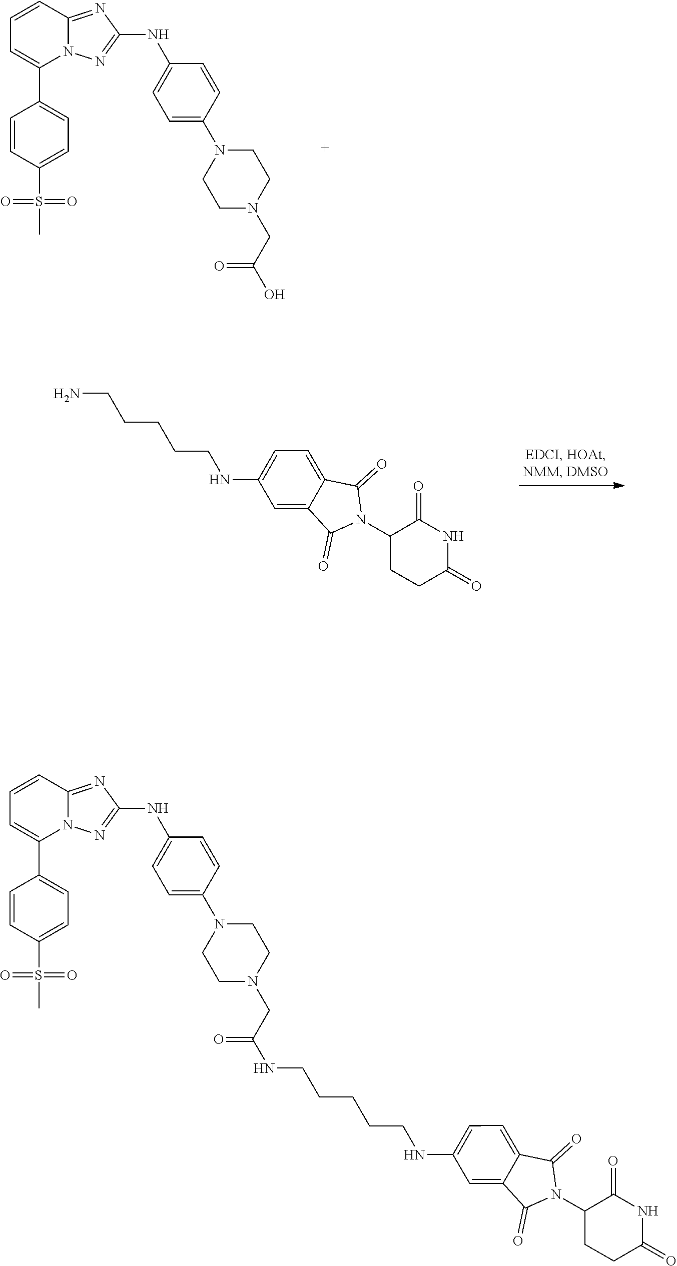 [SMILES: CS(=O)(=O)c1ccc(-c2cccc3nc(Nc4ccc(N5CCN(CC(=O)O)CC5)cc4)nn23)cc1 ; NCCCCCNc1ccc2c(c1)C(=O)N(C1CCC(=O)NC1=O)C2=O ; CS(=O)(=O)c1ccc(-c2cccc3nc(Nc4ccc(N5CCN(CC(=O)NCCCCCNc6ccc7c(c6)C(=O)N(C6CCC(=O)NC6=O)C7=O)CC5)cc4)nn23)cc1]

JA-204 was synthesized following the standard procedure for preparing JA-010 (8.1 mg, 67% yield). MS (ESI) m/z: 847.3 [M+H]⁺.

### Example 293. N-(4-((2-(2,6-Dioxopiperidin-3-yl)-1,3-dioxoisoindolin-5-yl)amino)butyl)-2-(4-(4-((5-(4-(methylsulfonyl)phenyl)-[1,2,4]triazolo[1,5-a]pyridin-2-yl)amino)phenyl)piperazin-1-yl)acetamide (JA-205)

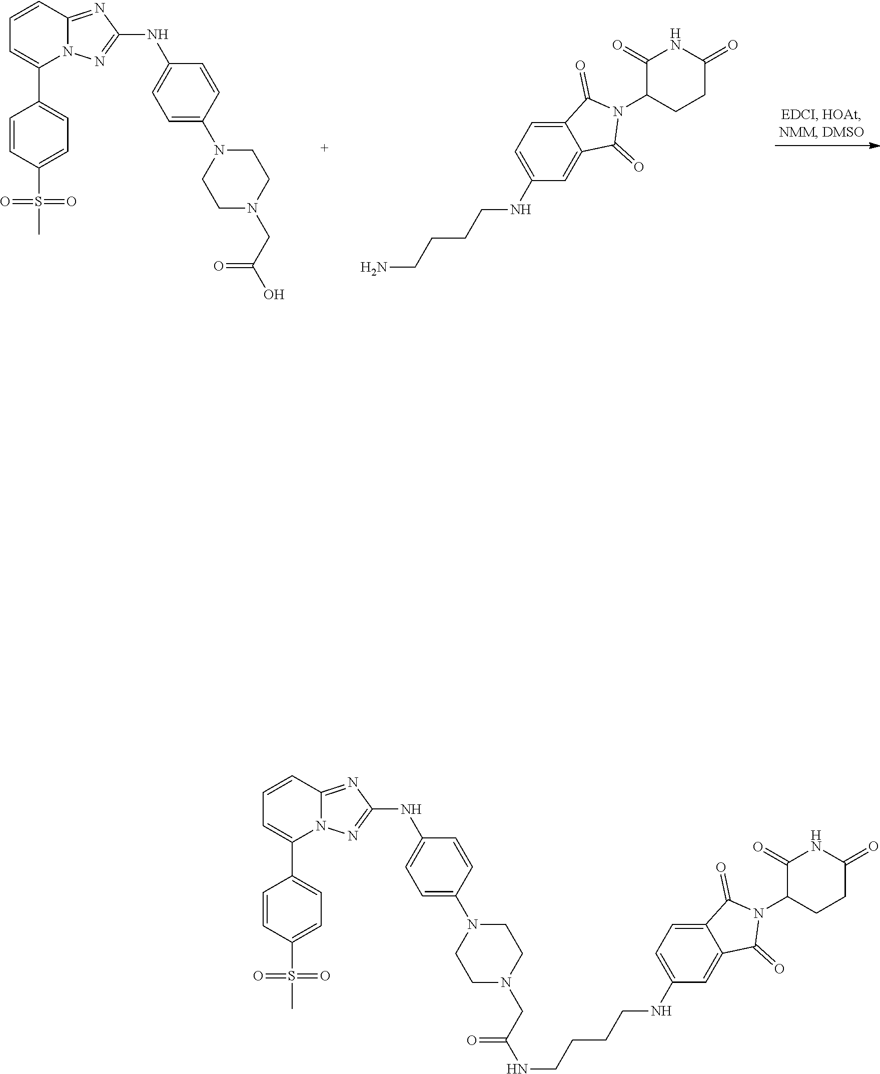 [SMILES: CS(=O)(=O)c1ccc(-c2cccc3nc(Nc4ccc(N5CCN(CC(=O)O)CC5)cc4)nn23)cc1 ; NCCCCNc1ccc2c(c1)C(=O)N(C1CCC(=O)NC1=O)C2=O ; CS(=O)(=O)c1ccc(-c2cccc3nc(Nc4ccc(N5CCN(CC(=O)NCCCCNc6ccc7c(c6)C(=O)N(C6CCC(=O)NC6=O)C7=O)CC5)cc4)nn23)cc1]

JA-205 was synthesized following the standard procedure for preparing JA-010 (8.2 mg, 64% yield). MS (ESI) m/z: 833.3 [M+H]⁺.

### Example 294. N-(14-((2-(2,6-Dioxopiperidin-3-yl)-1,3-dioxoisoindolin-5-yl)amino)-3,6,9,12-tetraoxatetradecyl)-2-(4-(4-((5-(4-(methylsulfonyl)phenyl)-[1,2,4]triazolo[1,5-a]pyridin-2-yl)amino)phenyl)piperazin-1-yl)acetamide (JA-206)

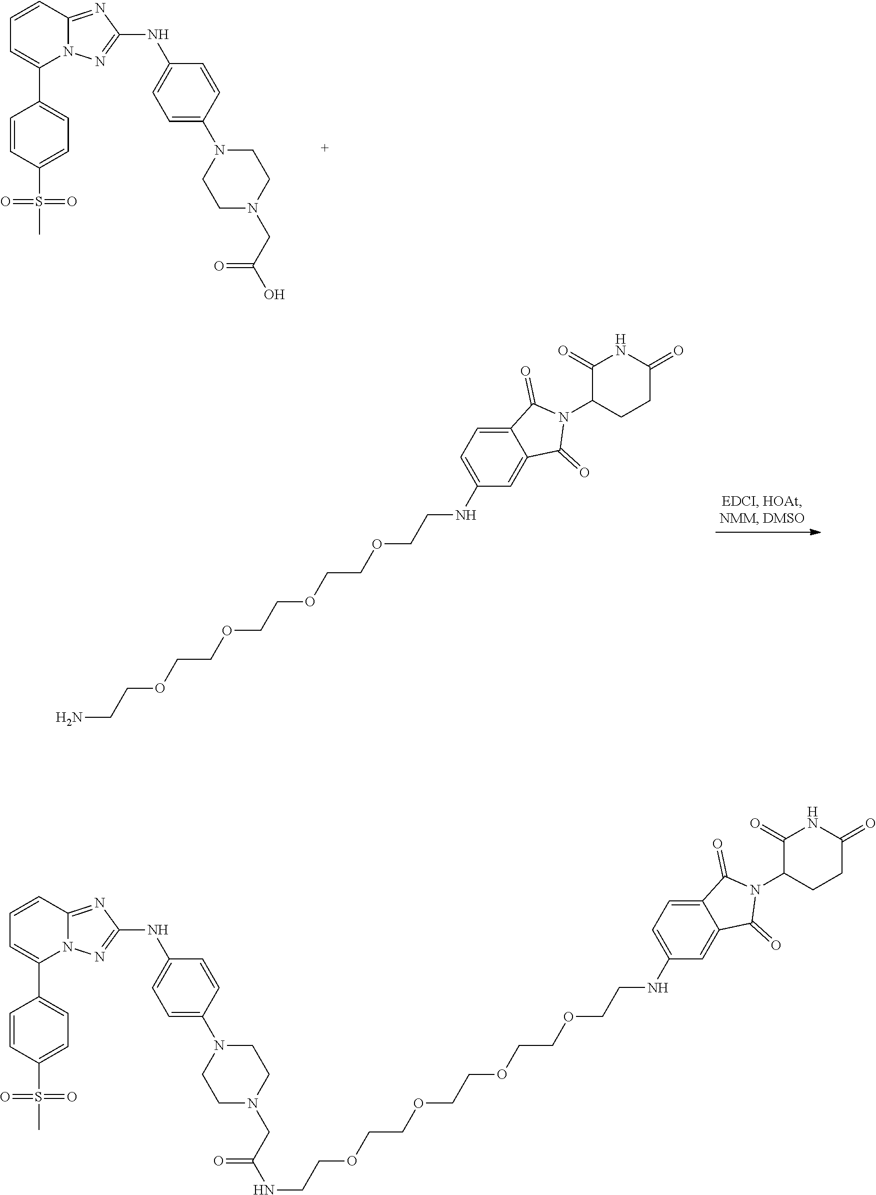 [SMILES: CS(=O)(=O)c1ccc(-c2cccc3nc(Nc4ccc(N5CCN(CC(=O)O)CC5)cc4)nn23)cc1 ; NCCOCCOCCOCCOCCNc1ccc2c(c1)C(=O)N(C1CCC(=O)NC1=O)C2=O ; CS(=O)(=O)c1ccc(-c2cccc3nc(Nc4ccc(N5CCN(CC(=O)NCCOCCOCCOCCOCCNc6ccc7c(c6)C(=O)N(C6CCC(=O)NC6=O)C7=O)CC5)cc4)nn23)cc1]

JA-206 was synthesized following the standard procedure for preparing JA-010 (6.8 mg, 68% yield). MS (ESI) m/z: 981.4 [M+H]⁺.

### Example 295. N-(2-((2-(2,6-Dioxopiperidin-3-yl)-1,3-dioxoisoindolin-5-yl)amino)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-207)

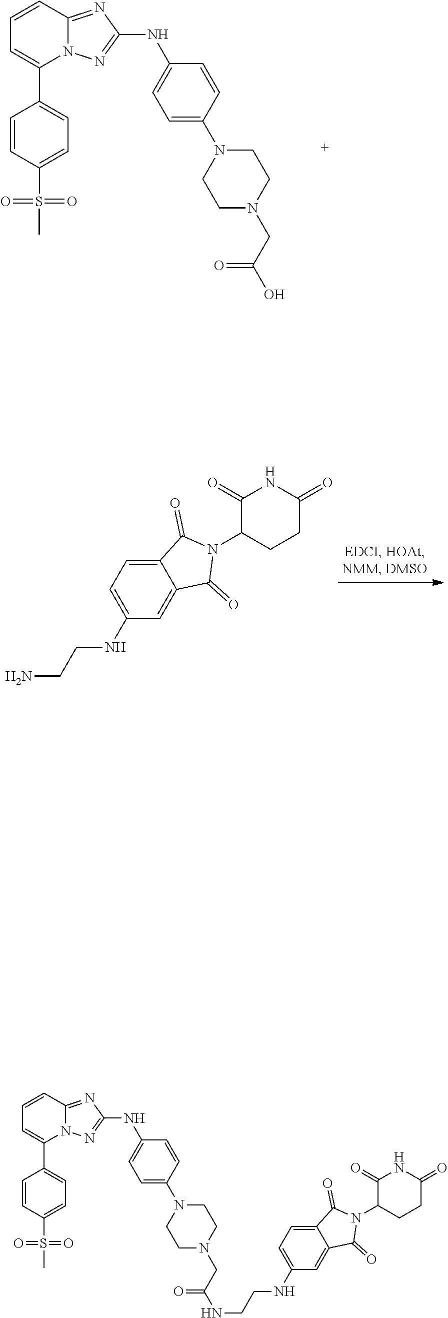 [SMILES: CS(=O)(=O)c1ccc(-c2cccc3nc(Nc4ccc(N5CCN(CC(=O)O)CC5)cc4)nn23)cc1 ; NCCNc1ccc2c(c1)C(=O)N(C1CCC(=O)NC1=O)C2=O ; CS(=O)(=O)c1ccc(-c2cccc3nc(Nc4ccc(N5CCN(CC(=O)NCCNc6ccc7c(c6)C(=O)N(C6CCC(=O)NC6=O)C7=O)CC5)cc4)nn23)cc1]

JA-207 was synthesized following the standard procedure for preparing JA-010 (6.8 mg, 68% yield). MS (ESI) m/z: 805.3 [M+H]⁺.

### Example 296. N-(2-(2-(2-((2-(2,6-Dioxopiperidin-3-yl)-1,3-dioxoisoindolin-5-yl)amino)ethoxy)ethoxy)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-208)

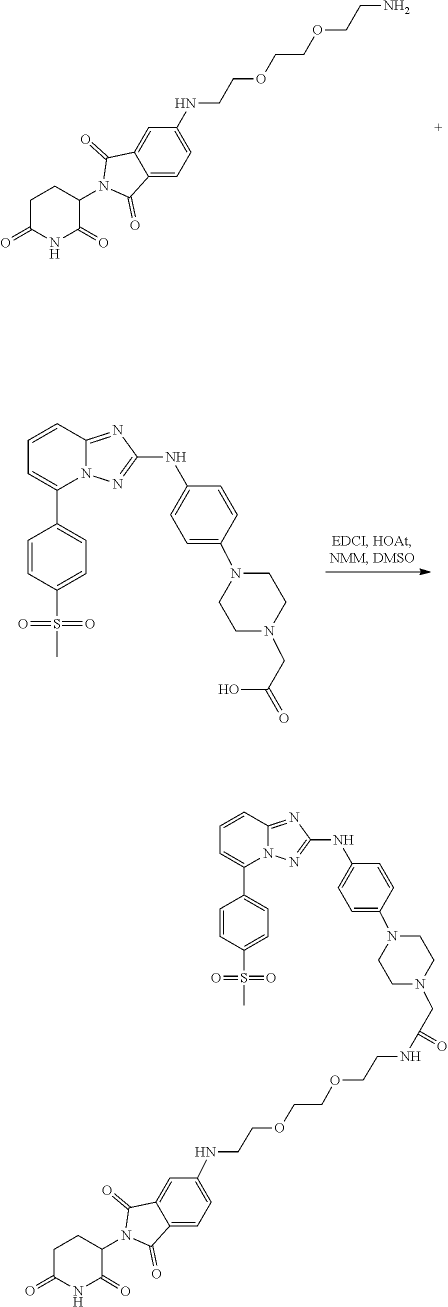 [SMILES: NCCOCCOCCNc1ccc2c(c1)C(=O)N(C1CCC(=O)NC1=O)C2=O ; CS(=O)(=O)c1ccc(-c2cccc3nc(Nc4ccc(N5CCN(CC(=O)O)CC5)cc4)nn23)cc1 ; CS(=O)(=O)c1ccc(-c2cccc3nc(Nc4ccc(N5CCN(CC(=O)NCCOCCOCCNc6ccc7c(c6)C(=O)N(C6CCC(=O)NC6=O)C7=O)CC5)cc4)nn23)cc1]

JA-208 was synthesized following the standard procedure for preparing JA-010 (7.7 mg, 69% yield). MS (ESI) m/z: 893.4 [M+H]⁺.

### Example 297. N-(2-(2-(2-(2-((2-(2,6-Dioxopiperidin-3-yl)-1,3-dioxoisoindolin-5-yl)amino)ethoxy)ethoxy)ethoxy)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-209)

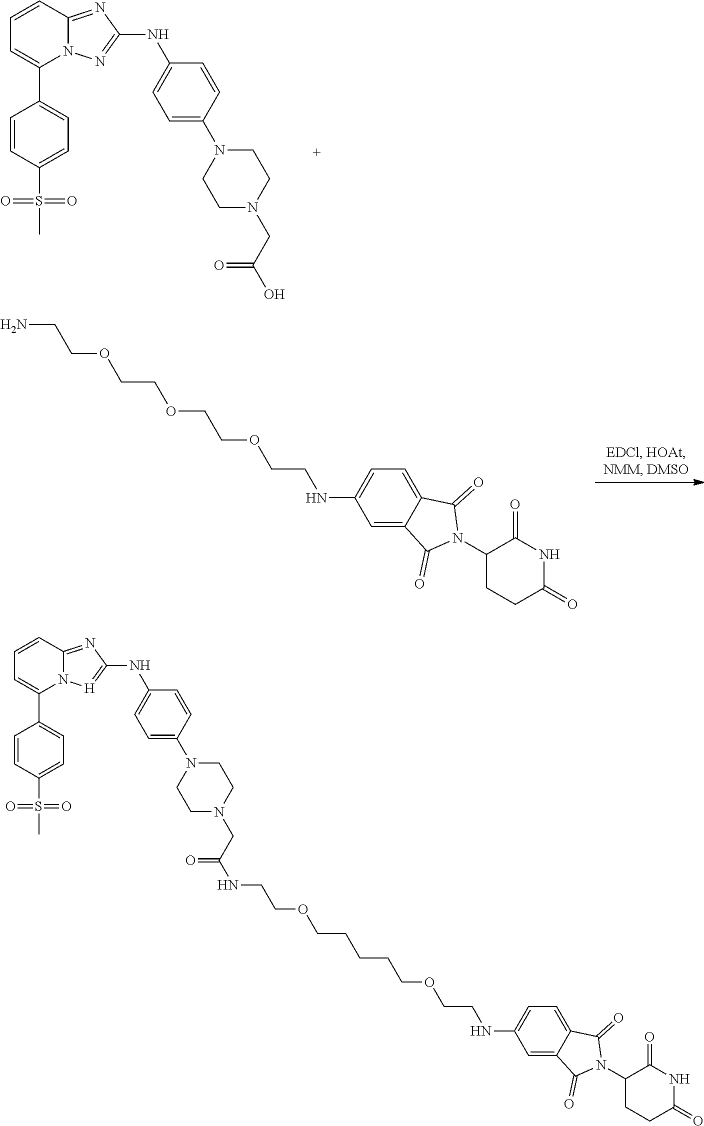

JA-209 was synthesized following the standard procedure for preparing JA-010 (7.7 mg, 64% yield). MS (ESI) m/z: 937.4 [M+H]⁺.

### Example 298. N-(3-((2-(2,6-Dioxopiperidin-3-yl)-1,3-dioxoisoindolin-5-yl)amino)propyl)-2-(4-(4-((5-(4-(methylsulfonyl)phenyl)-[1,2,4]triazolo[1,5-a]pyridin-2-yl)amino)phenyl)piperazin-1-yl)acetamide (JA-210)

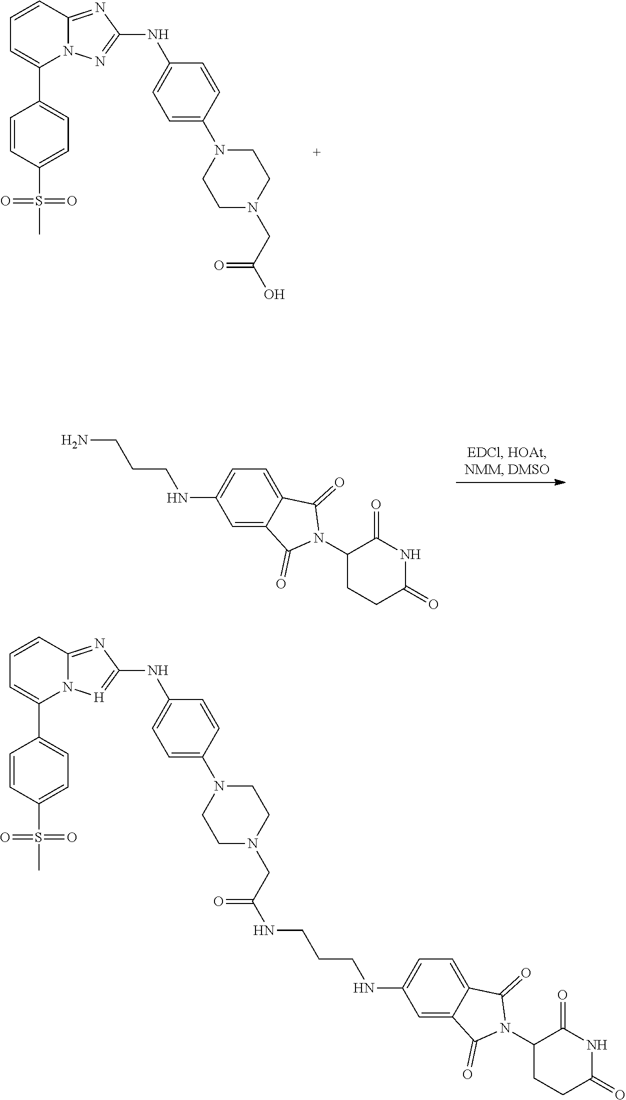

JA-210 was synthesized following the standard procedure for preparing JA-010 (6.9 mg, 71% yield). MS (ESI) m/z: 819.3 [M+H]⁺.

### Example 299. N-(17-((2-(2,6-Dioxopiperidin-3-yl)-1,3-dioxoisoindolin-5-yl)amino)-3,6,9,12,15-pentaoxaheptadecyl)-2-(4-(4-((5-(4-(methylsulfonyl)phenyl)-[1,2,4]triazolo[1,5-a]pyridin-2-yl)amino)phenyl)piperazin-1-yl)acetamide (JA-211)

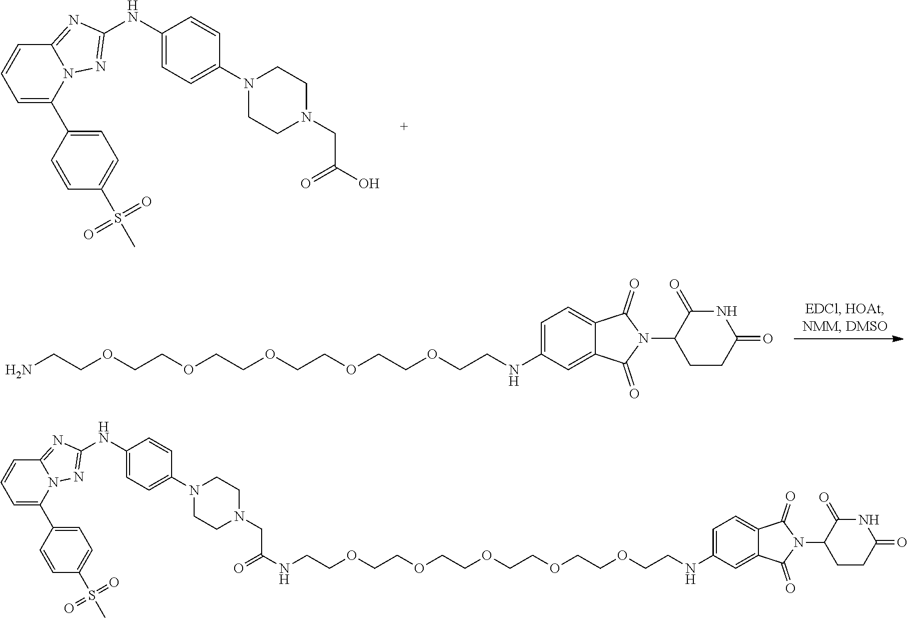 [SMILES: CS(=O)(=O)c1ccc(-c2cccc3nc(Nc4ccc(N5CCN(CC(=O)O)CC5)cc4)nn23)cc1 ; NCCOCCOCCOCCOCCOCCNc1ccc2c(c1)C(=O)N(C1CCC(=O)NC1=O)C2=O ; CS(=O)(=O)c1ccc(-c2cccc3nc(Nc4ccc(N5CCN(CC(=O)NCCOCCOCCOCCOCCOCCNc6ccc7c(c6)C(=O)N(C6CCC(=O)NC6=O)C7=O)CC5)cc4)nn23)cc1]

JA-211 was synthesized following the standard procedure for preparing JA-010 (7.2 mg, 72% yield). MS (ESI) m/z: 1025.3 [M+H]⁺.

### Example 300. N-(2-(2-((2-(2,6-Dioxopiperidin-3-yl)-1,3-dioxoisoindolin-5-yl)amino)ethoxy)ethyl)-2-(4-(4-((5-(4-(methylsulfonyl)phenyl)-[1,2,4]triazolo[1,5-a]pyridin-2-yl)amino)phenyl)piperazin-1-yl)acetamide (JA-212)

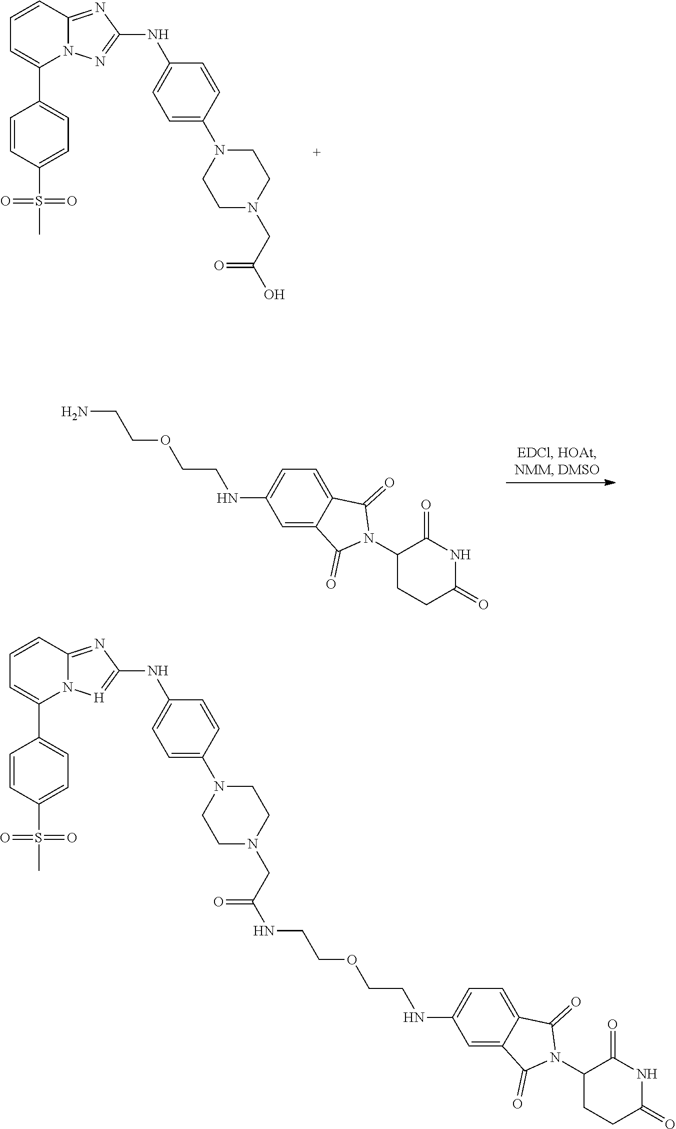

JA-212 was synthesized following the standard procedure for preparing JA-010 (6.2 mg, 72% yield). MS (ESI) m/z: 849.3 [M+H]⁺.

### Example 301. N-(8-((2-(2,6-Dioxopiperidin-3-yl)-1,3-dioxoisoindolin-5-yl)amino)octyl)-2-(4-(4-((5-(4-(methylsulfonyl)phenyl)-[1,2,4]triazolo[1,5-a]pyridin-2-yl)amino)phenyl)piperazin-1-yl)acetamide (JA-213)

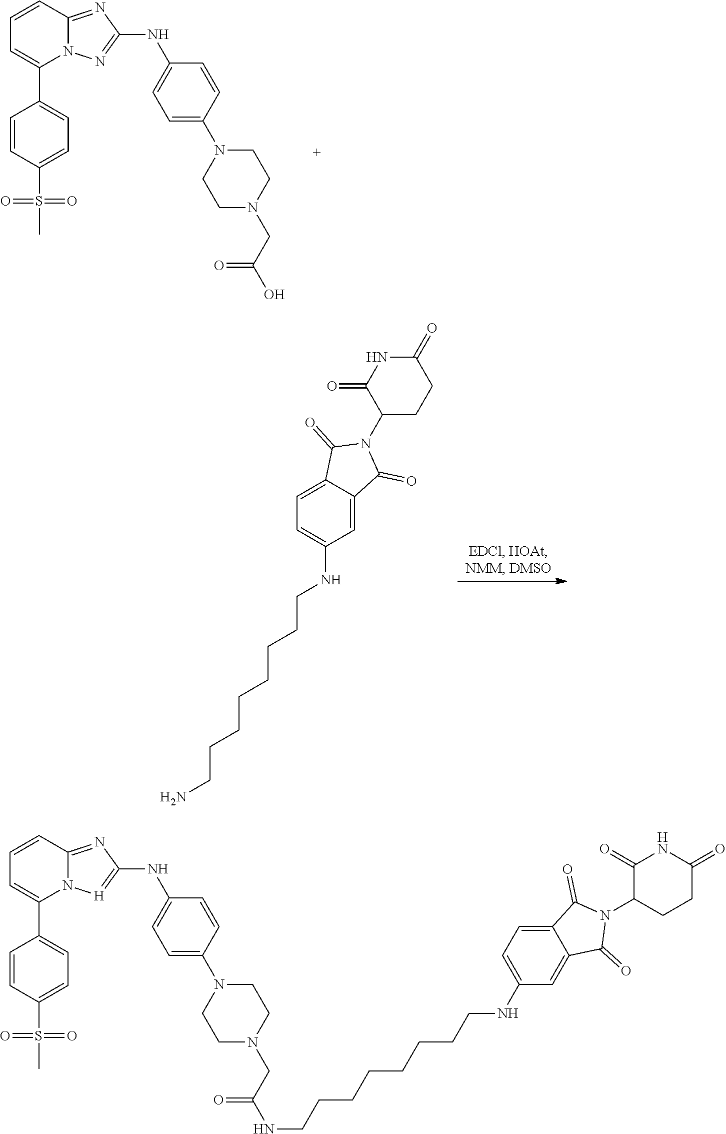

JA-213 was synthesized following the standard procedure for preparing JA-010 (7.3 mg, 62% yield). MS (ESI) m/z: 889.3 [M+H]⁺.

### Example 302. N-(7-((2-(2,6-Dioxopiperidin-3-yl)-1,3-dioxoisoindolin-5-yl)amino)heptyl)-2-(4-(4-((5-(4-(methylsulfonyl)phenyl)-[1,2,4]triazolo[1,5-a]pyridin-2-yl)amino)phenyl)piperazin-1-yl)acetamide (JA-214)

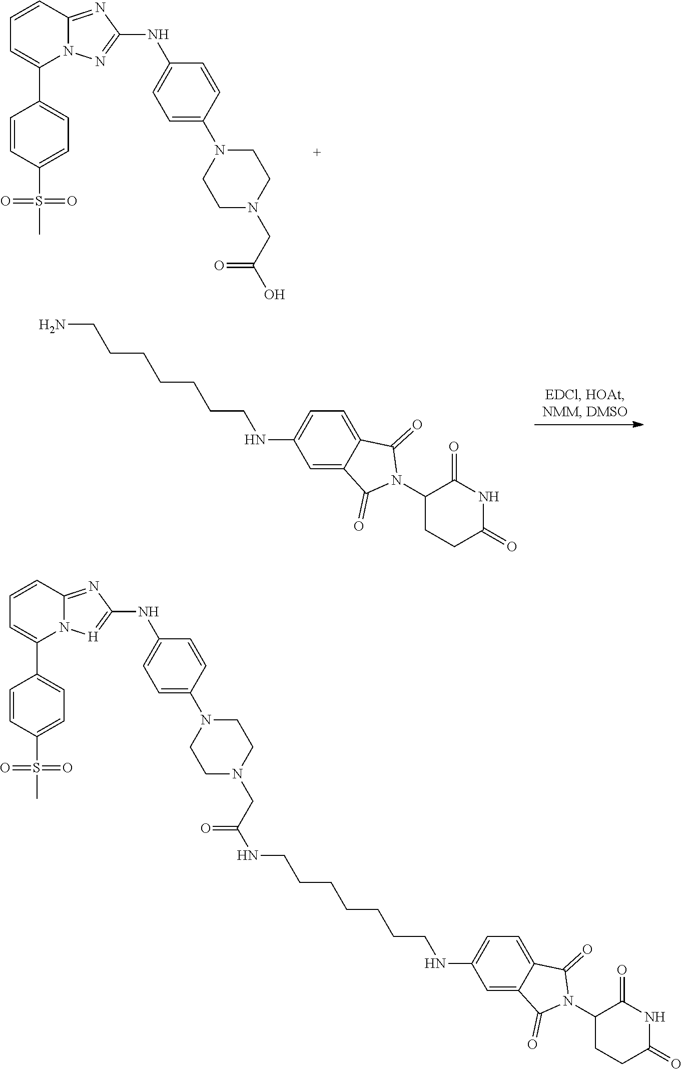

JA-214 was synthesized following the standard procedure for preparing JA-010 (7.1 mg, 66% yield). MS (ESI) m/z: 875.4 [M+H]⁺.

### Example 303. N-(6-((2-(2,6-dioxopiperidin-3-yl)-1,3-dioxoisoindolin-5-yl)amino)hexyl)-2-(4-(4-((5-(4-(methylsulfonyl)phenyl)-[1,2,4]triazolo[1,5-a]pyridin-2-yl)amino)phenyl)piperazin-1-yl)acetamide (JA-215)

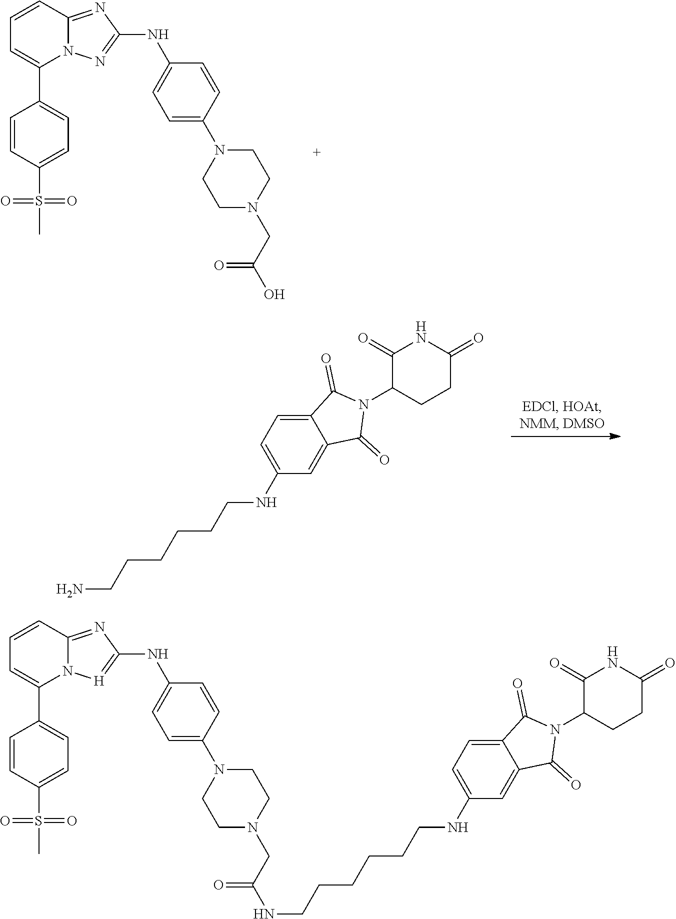

JA-215 was synthesized following the standard procedure for preparing JA-010 (7.3 mg, 67% yield). MS (ESI) m/z: 861.4 [M+H]⁺.

### Example 304. 2-(2,6-Dioxopiperidin-3-yl)-5-((4-(4-(4-((5-(4-(methylsulfonyl)phenyl)-[1,2,4]triazolo[1,5-a]pyridin-2-yl)amino)phenyl)piperazin-1-yl)-4-oxobutyl)amino)isoindoline-1,3-dione (JA-216)

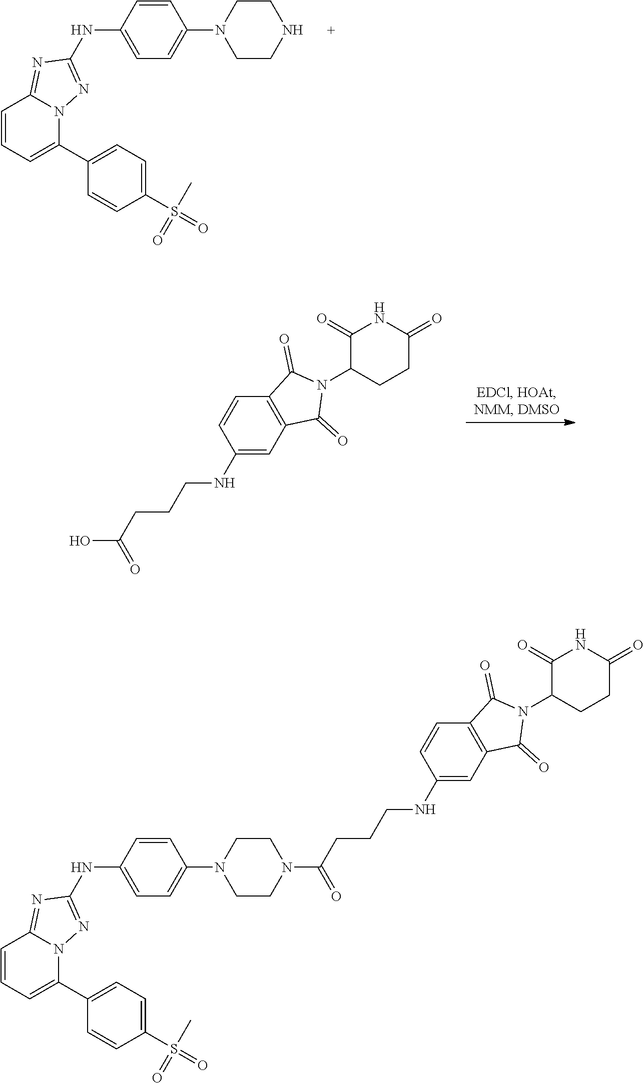 [SMILES: CS(=O)(=O)c1ccc(-c2cccc3nc(Nc4ccc(N5CCNCC5)cc4)nn23)cc1 ; O=C(O)CCCNc1ccc2c(c1)C(=O)N(C1CCC(=O)NC1=O)C2=O ; CS(=O)(=O)c1ccc(-c2cccc3nc(Nc4ccc(N5CCN(C(=O)CCCNc6ccc7c(c6)C(=O)N(C6CCC(=O)NC6=O)C7=O)CC5)cc4)nn23)cc1]

JA-216 was synthesized following the standard procedure for preparing JA-010 (8.4 mg, 69% yield). MS (ESI) m/z: 790.3 [M+H]⁺.

### Example 305. (2S,4R)-1-((S)-2-(2-(2-(2-(4-(4-((4-((3-(N-(tert-butyl)sulfamoyl)phenyl)amino)-5-methylpyrimidin-2-yl)amino)phenyl)piperazin-1-yl)acetamido)ethoxy)acetamido)-3,3-dimethylbutanoyl)-4-hydroxy-N-(4-(4-methylthiazol-5-yl)benzyl)pyrrolidine-2-carboxamide (JA-217)

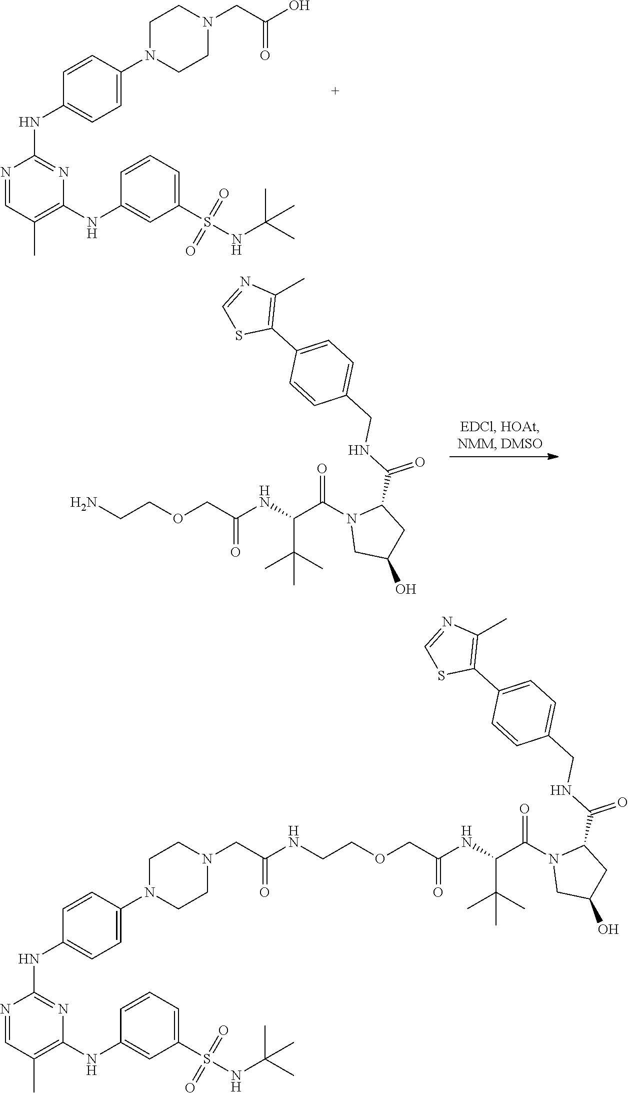

JA-217 was synthesized following the standard procedure for preparing JA-015 (4 mg, 31% yield). MS (ESI) m/z: 1067.9 [M+H]⁺.

### Example 306. (2S,4R)-1-((S)-2-(3-(2-(2-(4-(4-((4-((3-(N-(tert-butyl)sulfamoyl)phenyl)amino)-5-methylpyrimidin-2-yl)amino)phenyl)piperazin-1-yl)acetamido)ethoxy)propanamido)-3,3-dimethylbutanoyl)-4-hydroxy-N-(4-(4-methylthiazol-5-yl)benzyl)pyrrolidine-2-carboxamide (JA-218)

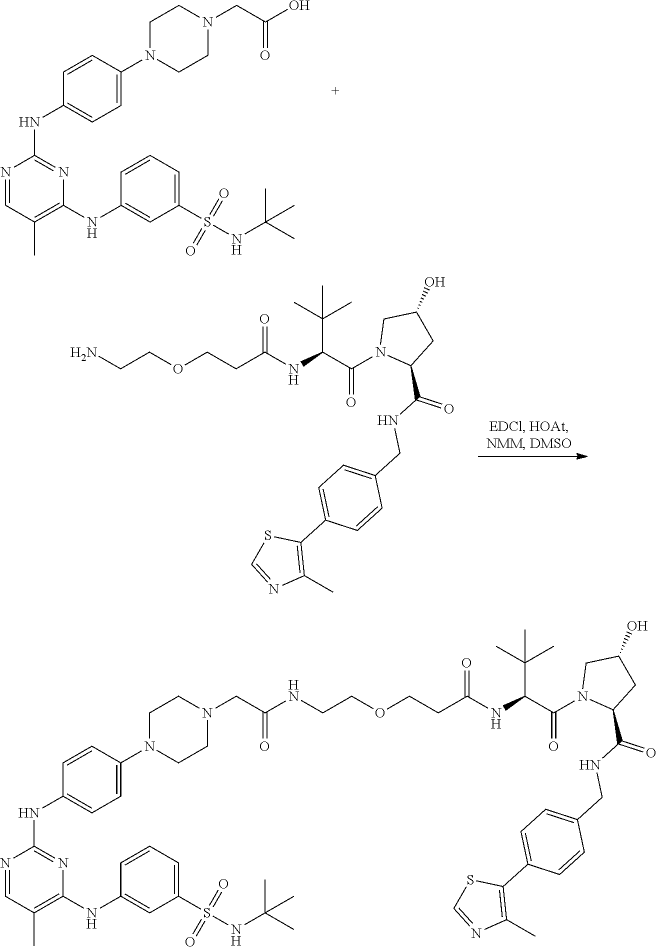 [SMILES: Cc1cnc(Nc2ccc(N3CCN(CC(=O)O)CC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCOCCN)C(C)(C)C)cc1 ; Cc1cnc(Nc2ccc(N3CCN(CC(=O)NCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-218 was synthesized following the standard procedure for preparing JA-015 (5 mg, 38% yield). MS (ESI) m/z: 1082.0 [M+H]⁺.

### Example 307. (2S,4R)-1-((S)-14-(tert-butyl)-1-(4-(4-((4-((3-(N-(tert-butyl)sulfamoyl)phenyl)amino)-5-methylpyrimidin-2-yl)amino)phenyl)piperazin-1-yl)-2,12-dioxo-6,9-dioxa-3,13-diazapentadecan-15-oyl)-4-hydroxy-N-(4-(4-methylthiazol-5-yl)benzyl)pyrrolidine-2-carboxamide (JA-219)

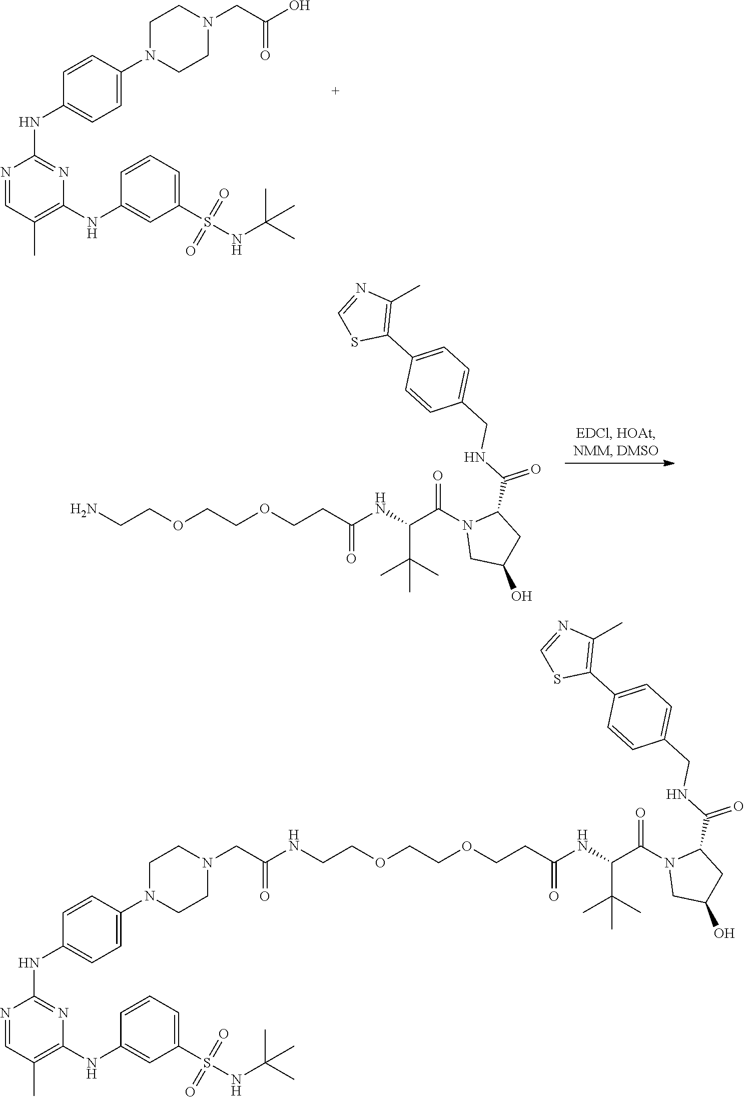 [SMILES: Cc1cnc(Nc2ccc(N3CCN(CC(=O)O)CC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCOCCOCCN)C(C)(C)C)cc1 ; Cc1cnc(Nc2ccc(N3CCN(CC(=O)NCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-219 was synthesized following the standard procedure for preparing JA-015 (3 mg, 21% yield). MS (ESI) m/z: 1126.2 [M+H]⁺.

### Example 308. (2S,4R)-1-((S)-2-(tert-butyl)-17-(4-(4-((4-((3-(N-(tert-butyl)sulfamoyl)phenyl)amino)-5-methylpyrimidin-2-yl)amino)phenyl)piperazin-1-yl)-4,16-dioxo-6,9,12-trioxa-3,15-diazaheptadecanoyl)-4-hydroxy-N-(4-(4-methylthiazol-5-yl)benzyl)pyrrolidine-2-carboxamide (JA-220)

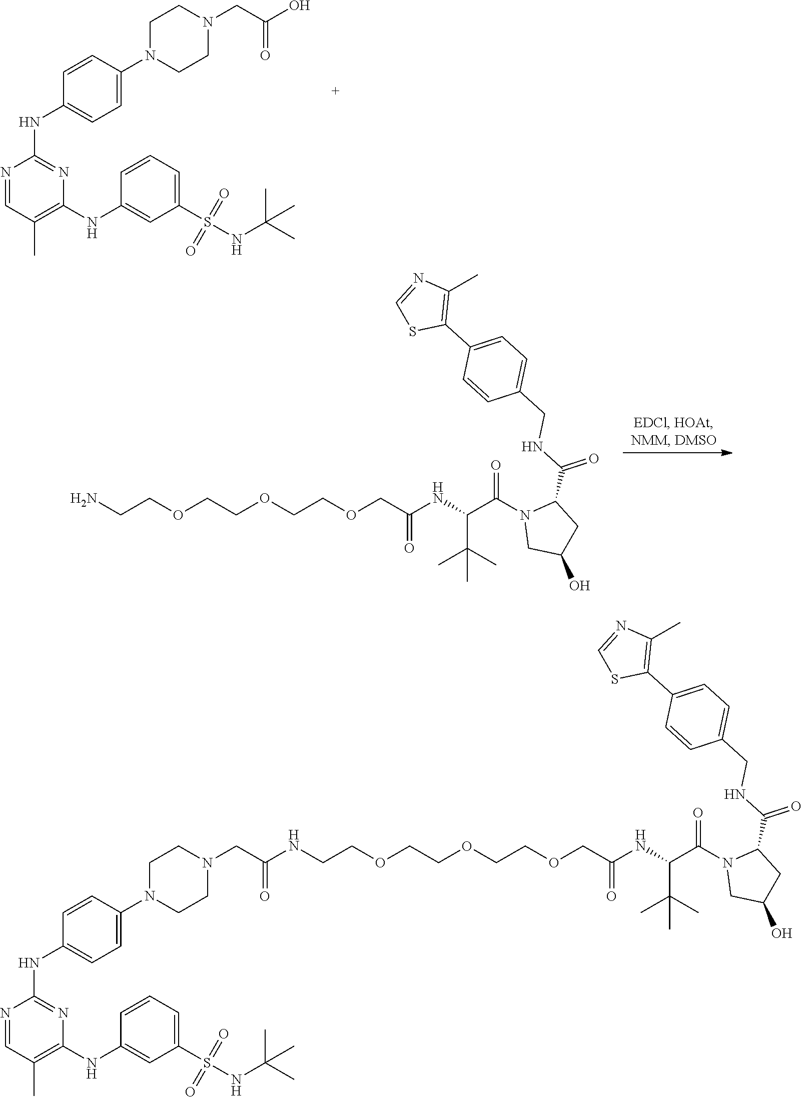 [SMILES: Cc1cnc(Nc2ccc(N3CCN(CC(=O)O)CC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)COCCOCCOCCN)C(C)(C)C)cc1 ; Cc1cnc(Nc2ccc(N3CCN(CC(=O)NCCOCCOCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-220 was synthesized following the standard procedure for preparing JA-015 (3 mg, 22% yield). MS (ESI) m/z: 1156.3 [M+H]⁺.

### Example 309. (2S,4R)-1-((S)-17-(tert-butyl)-1-(4-(4-((4-((3-(N-(tert-butyl)sulfamoyl)phenyl)amino)-5-methylpyrimidin-2-yl)amino)phenyl)piperazin-1-yl)-2,15-dioxo-6,9,12-trioxa-3,16-diazaoctadecan-18-oyl)-4-hydroxy-N-(4-(4-methylthiazol-5-yl)benzyl)pyrrolidine-2-carboxamide (JA-221)

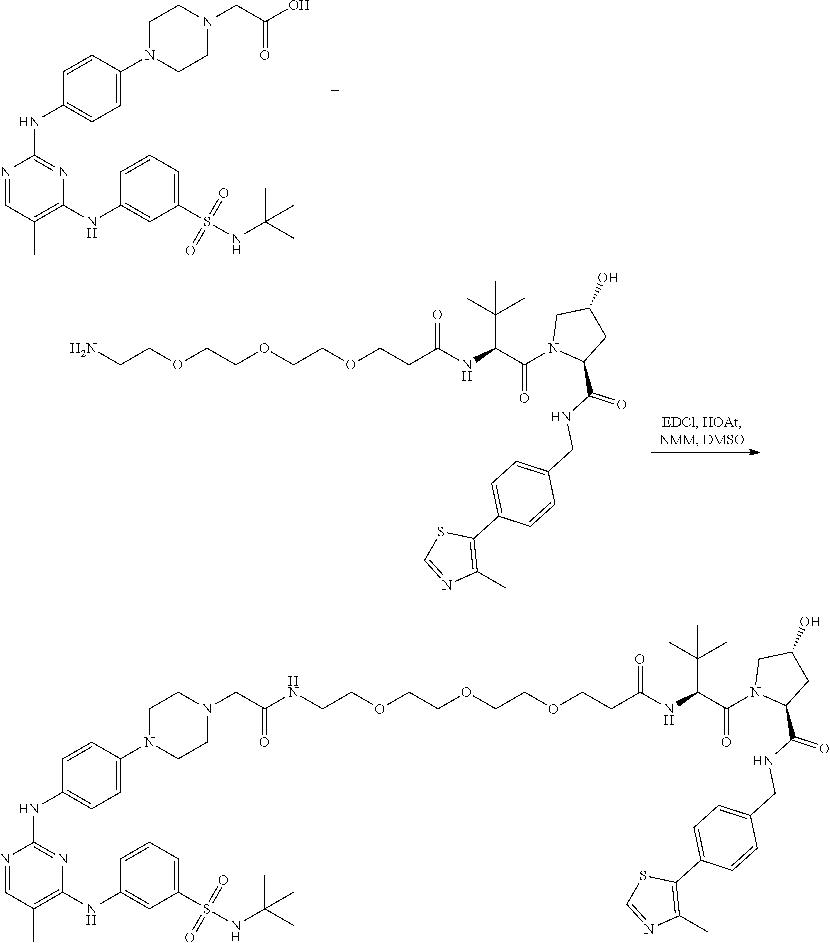 [SMILES: Cc1cnc(Nc2ccc(N3CCN(CC(=O)O)CC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCOCCOCCOCCN)C(C)(C)C)cc1 ; Cc1cnc(Nc2ccc(N3CCN(CC(=O)NCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-221 was synthesized following the standard procedure for preparing JA-015 (6 mg, 43% yield). MS (ESI) m/z: 1170.1 [M+H]⁺.

### Example 310. (2S,4R)-1-((S)-20-(tert-butyl)-1-(4-(4-((4-((3-(N-(tert-butyl)sulfamoyl)phenyl)amino)-5-methylpyrimidin-2-yl)amino)phenyl)piperazin-1-yl)-2,18-dioxo-6,9,12,15-tetraoxa-3,19-diazahenicosan-21-oyl)-4-hydroxy-N-(4-(4-methylthiazol-5-yl)benzyl)pyrrolidine-2-carboxamide (JA-222)

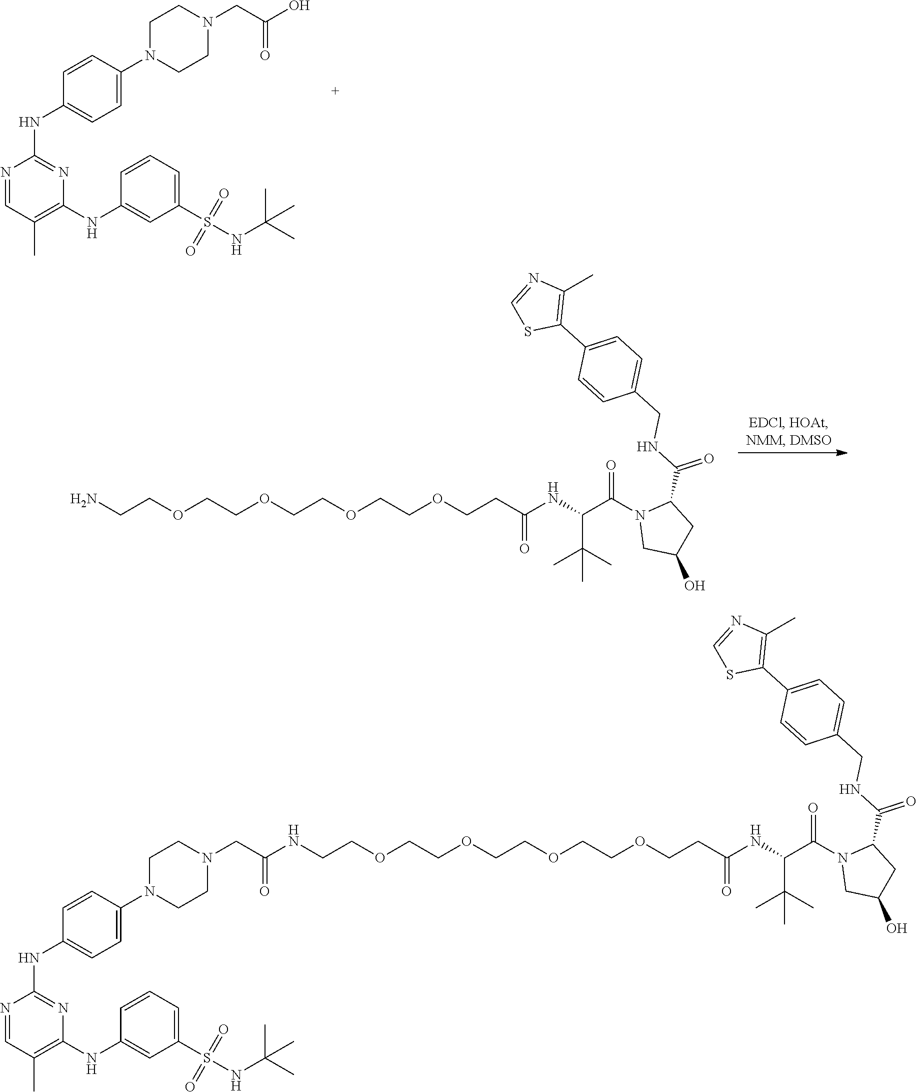 [SMILES: Cc1cnc(Nc2ccc(N3CCN(CC(=O)O)CC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCOCCOCCOCCOCCN)C(C)(C)C)cc1 ; Cc1cnc(Nc2ccc(N3CCN(CC(=O)NCCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-222 was synthesized following the standard procedure for preparing JA-015 (4 mg, 28% yield). MS (ESI) m/z: 1214.3 [M+H]⁺.

### Example 311. (2S,4R)-1-((S)-23-(tert-butyl)-1-(4-(4-((4-((3-(N-(tert-butyl)sulfamoyl)phenyl)amino)-5-methylpyrimidin-2-yl)amino)phenyl)piperazin-1-yl)-2,21-dioxo-6,9,12,15,18-pentaoxa-3,22-diazatetracosan-24-oyl)-4-hydroxy-N-(4-(4-methylthiazol-5-yl)benzyl)pyrrolidine-2-carboxamide (JA-223)

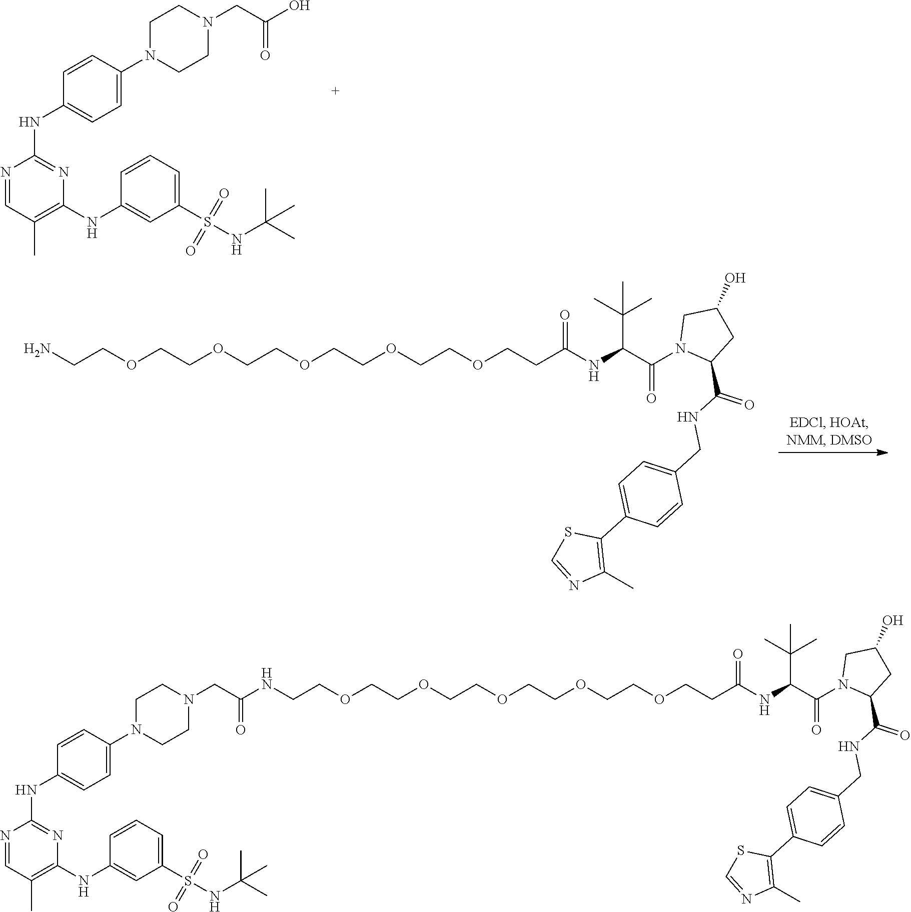 [SMILES: Cc1cnc(Nc2ccc(N3CCN(CC(=O)O)CC3)cc2)nc1Nc1cccc(S(=O)(=O)NC(C)(C)C)c1 ; Cc1ncsc1-c1ccc(CNC(=O)[C@@H]2C[C@@H](O)CN2C(=O)[C@@H](NC(=O)CCOCCOCCOCCOCCOCCN)C(C)(C)C)cc1 ; Cc1cnc(Nc2ccc(N3CCN(CC(=O)NCCOCCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1]

JA-223 was synthesized following the standard procedure for preparing JA-015 (6 mg, 40% yield). MS (ESI) m/z: 1258.4 [M+H]⁺.

### Example 312. 2-(4-(4-((4-((3-(N-(tert-butyl)sulfamoyl)phenyl)amino)-5-methylpyrimidin-2-yl)amino)phenyl)piperazin-1-yl)-N-(17-((2-(2,6-dioxopiperidin-3-yl)-1,3-dioxoisoindolin-4-yl)amino)-3,6,9,12,15-pentaoxaheptadecyl)acetamide (JA-224)

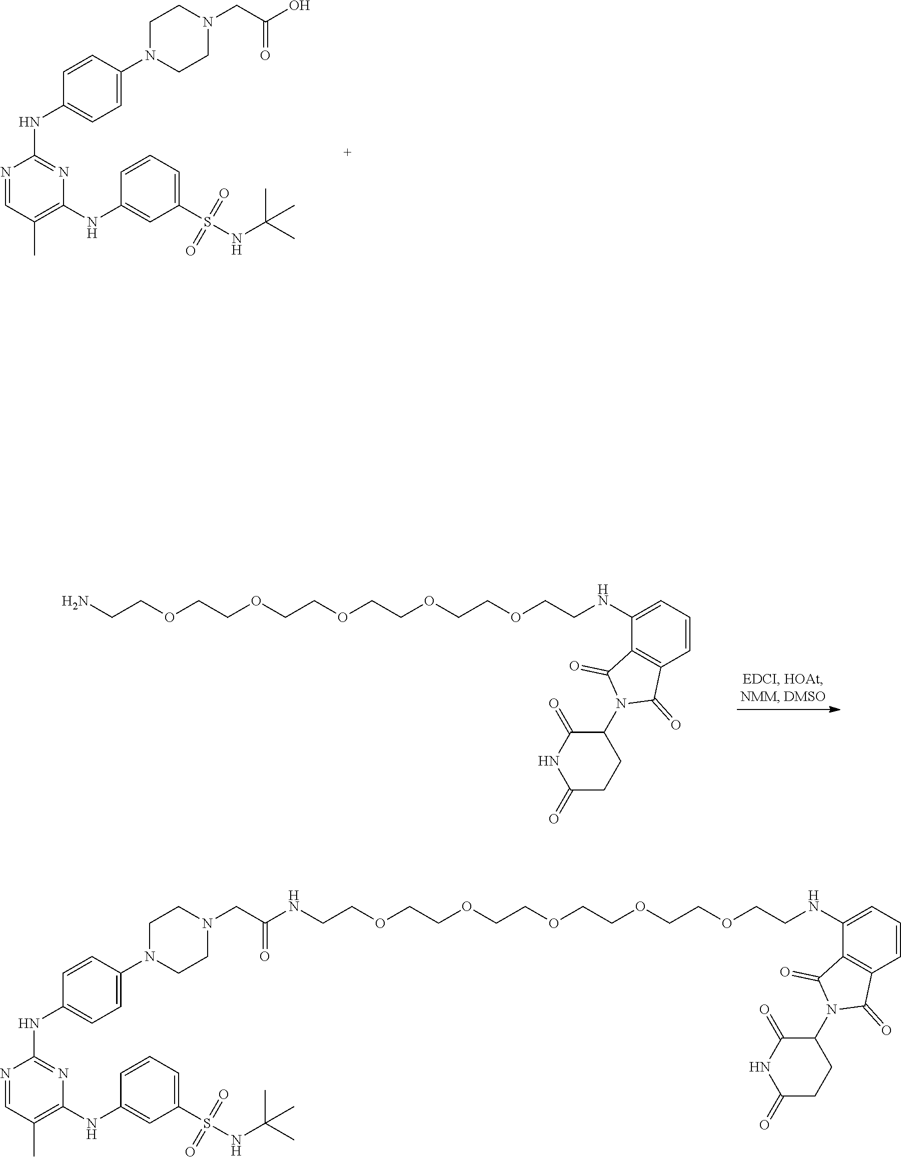 [SMILES: Cc1cnc(Nc2ccc(N3CCN(CC(=O)O)CC3)cc2)nc1Nc1cccc(S(=O)(=O)NC(C)(C)C)c1 ; NCCOCCOCCOCCOCCOCCNc1cccc2c1C(=O)N(C1CCC(=O)NC1=O)C2=O ; Cc1cnc(Nc2ccc(N3CCN(CC(=O)NCCOCCOCCOCCOCCOCCNc4cccc5c4C(=O)N(C4CCC(=O)NC4=O)C5=O)CC3)cc2)nc1Nc1cccc(S(=O)(=O)NC(C)(C)C)c1]

JA-224 was synthesized following the standard procedure for preparing JA-015 (1.3 mg, 6% yield). MS (ESI) m/z: 1073.0 [M+H]⁺.

### Example 313. N-(tert-butyl)-3-((2-((4-(4-((2-(2,6-dioxopiperidin-3-yl)-1,3-dioxoisoindolin-5-yl)glycyl)piperazin-1-yl)phenyl)amino)-5-methylpyrimidin-4-yl)amino)benzenesulfonamide (JA-225)

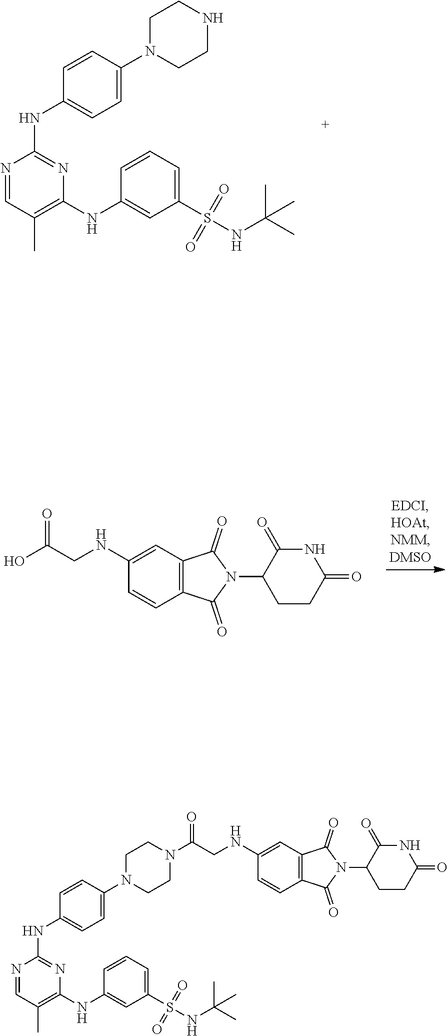 [SMILES: Cc1cnc(Nc2ccc(N3CCNCC3)cc2)nc1Nc1cccc(S(=O)(=O)NC(C)(C)C)c1 ; O=C(O)CNc1ccc2c(c1)C(=O)N(C1CCC(=O)NC1=O)C2=O ; Cc1cnc(Nc2ccc(N3CCN(C(=O)CNc4ccc5c(c4)C(=O)N(C4CCC(=O)NC4=O)C5=O)CC3)cc2)nc1Nc1cccc(S(=O)(=O)NC(C)(C)C)c1]

JA-225 was synthesized following the standard procedure for preparing JA-015 (9 mg, 59% yield). MS (ESI) m/z: 809.7 [M+H]⁺.

### Example 314. N-(tert-butyl)-3-((2-((4-(4-(3-((2-(2,6-dioxopiperidin-3-yl)-1,3-dioxoisoindolin-5-yl)amino)propanoyl)piperazin-1-yl)phenyl)amino)-5-methylpyrimidin-4-yl)amino)benzenesulfonamide (JA-226)

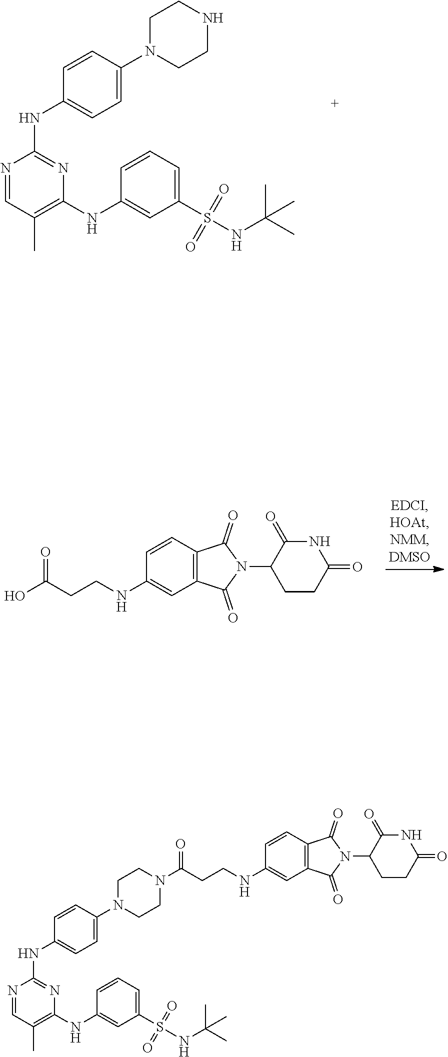 [SMILES: Cc1cnc(Nc2ccc(N3CCNCC3)cc2)nc1Nc1cccc(S(=O)(=O)NC(C)(C)C)c1 ; O=C(O)CCNc1ccc2c(c1)C(=O)N(C1CCC(=O)NC1=O)C2=O ; Cc1cnc(Nc2ccc(N3CCN(C(=O)CCNc4ccc5c(c4)C(=O)N(C4CCC(=O)NC4=O)C5=O)CC3)cc2)nc1Nc1cccc(S(=O)(=O)NC(C)(C)C)c1]

JA-226 was synthesized following the standard procedure for preparing JA-015 (9 mg, 58% yield). MS (ESI) m/z: 823.7 [M+H]⁺.

### Example 315. N-(tert-butyl)-3-((2-((4-(4-(4-((2-(2,6-dioxopiperidin-3-yl)-1,3-dioxoisoindolin-5-yl)amino)butanoyl)piperazin-1-yl)phenyl)amino)-5-methylpyrimidin-4-yl)amino)benzenesulfonamide (JA-227)

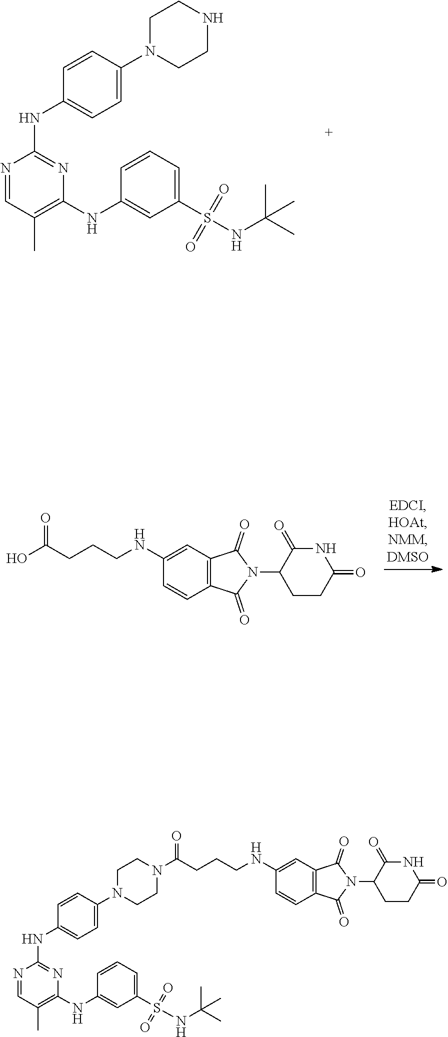 [SMILES: Cc1cnc(Nc2ccc(N3CCNCC3)cc2)nc1Nc1cccc(S(=O)(=O)NC(C)(C)C)c1 ; O=C(O)CCCNc1ccc2c(c1)C(=O)N(C1CCC(=O)NC1=O)C2=O ; Cc1cnc(Nc2ccc(N3CCN(C(=O)CCCNc4ccc5c(c4)C(=O)N(C4CCC(=O)NC4=O)C5=O)CC3)cc2)nc1Nc1cccc(S(=O)(=O)NC(C)(C)C)c1]

JA-227 was synthesized following the standard procedure for preparing JA-015 (9.6 mg, 61% yield). MS (ESI) m/z: 837.7 [M+H]⁺.

### Example 316. N-(tert-butyl)-3-((2-((4-(4-(5-((2-(2,6-dioxopiperidin-3-yl)-1,3-dioxoisoindolin-5-yl)amino)pentanoyl)piperazin-1-yl)phenyl)amino)-5-methylpyrimidin-4-yl)amino)benzenesulfonamide (JA-228)

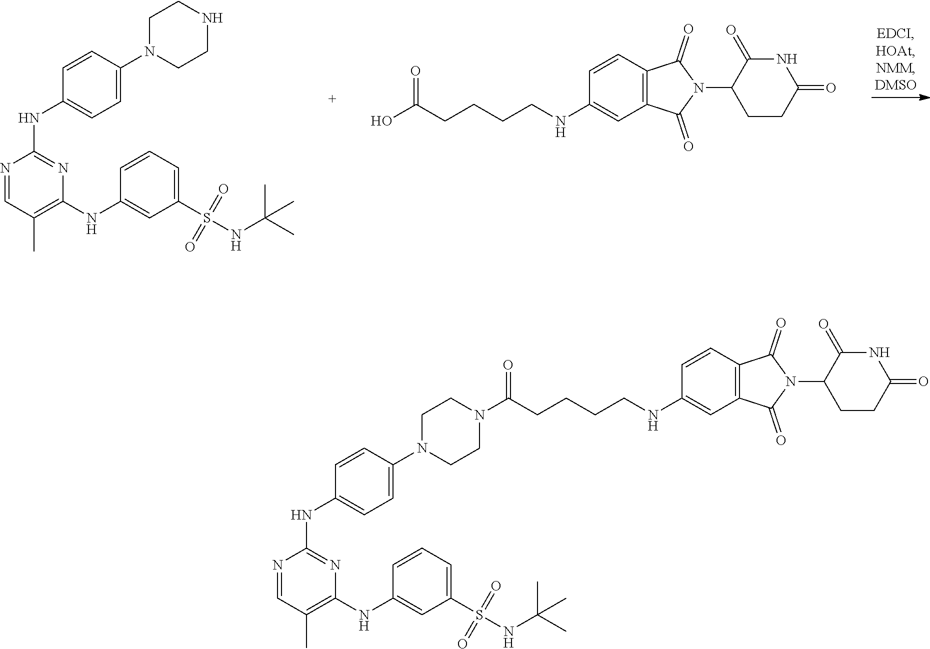 [SMILES: Cc1cnc(Nc2ccc(N3CCNCC3)cc2)nc1Nc1cccc(S(=O)(=O)NC(C)(C)C)c1 ; O=C(O)CCCCNc1ccc2c(c1)C(=O)N(C1CCC(=O)NC1=O)C2=O ; Cc1cnc(Nc2ccc(N3CCN(C(=O)CCCCNc4ccc5c(c4)C(=O)N(C4CCC(=O)NC4=O)C5=O)CC3)cc2)nc1Nc1cccc(S(=O)(=O)NC(C)(C)C)c1]

JA-228 was synthesized following the standard procedure for preparing JA-015 (9.5 mg, 59% yield). MS (ESI) m/z: 851.8 [M+H]⁺.

### Example 317. N-(tert-butyl)-3-((2-((4-(4-(6-((2-(2,6-dioxopiperidin-3-yl)-1,3-dioxoisoindolin-5-yl)amino)hexanoyl)piperazin-1-yl)phenyl)amino)-5-methylpyrimidin-4-yl)amino)benzenesulfonamide (JA-229)

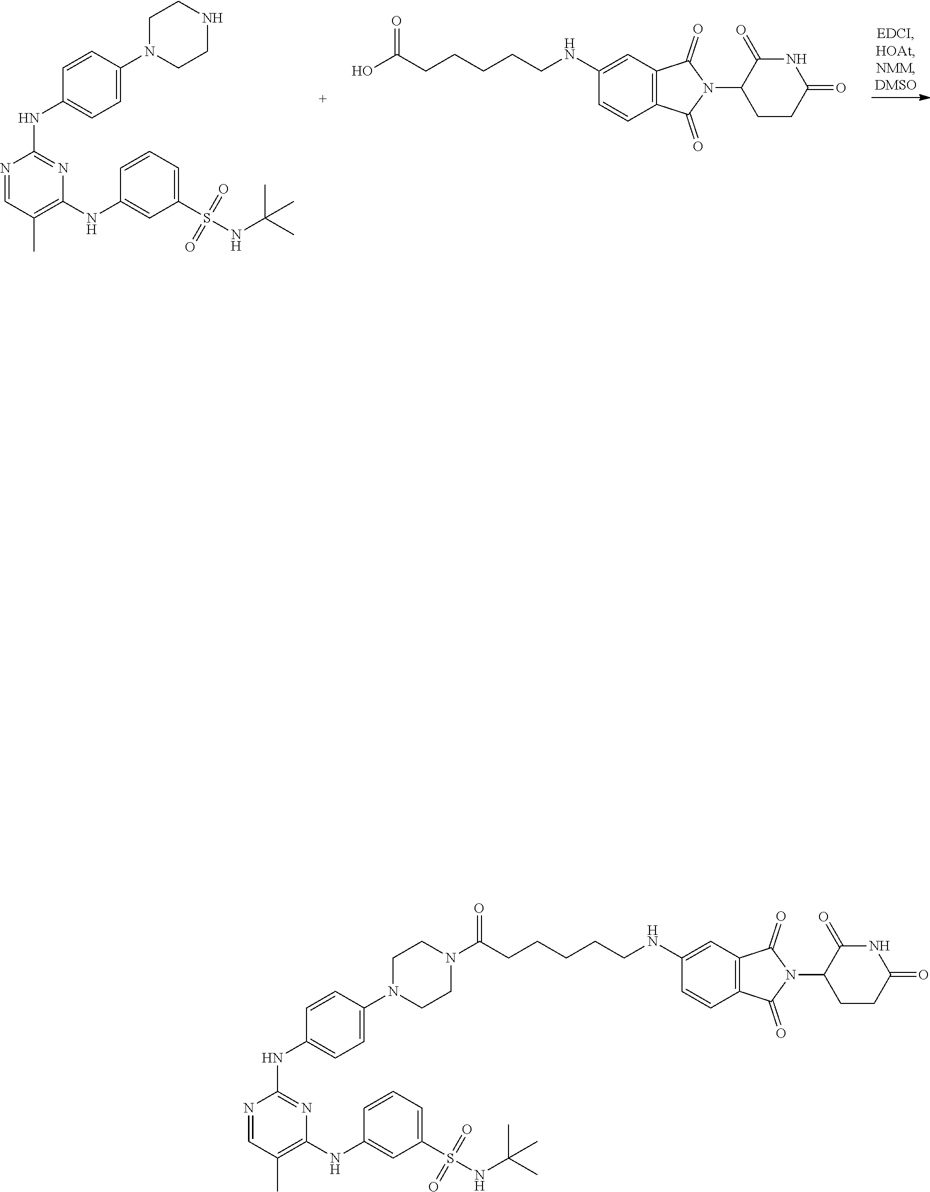 [SMILES: Cc1cnc(Nc2ccc(N3CCNCC3)cc2)nc1Nc1cccc(S(=O)(=O)NC(C)(C)C)c1 ; O=C(O)CCCCCNc1ccc2c(c1)C(=O)N(C1CCC(=O)NC1=O)C2=O ; Cc1cnc(Nc2ccc(N3CCN(C(=O)CCCCCNc4ccc5c(c4)C(=O)N(C4CCC(=O)NC4=O)C5=O)CC3)cc2)nc1Nc1cccc(S(=O)(=O)NC(C)(C)C)c1]

JA-229 was synthesized following the standard procedure for preparing JA-015 (9 mg, 55% yield). MS (ESI) m/z: 865.7 [M+H]⁺.

### Example 318. N-(tert-butyl)-3-((2-((4-(4-(7-((2-(2,6-dioxopiperidin-3-yl)-1,3-dioxoisoindolin-5-yl)amino)heptanoyl)piperazin-1-yl)phenyl)amino)-5-methylpyrimidin-4-yl)amino)benzenesulfonamide (JA-230)

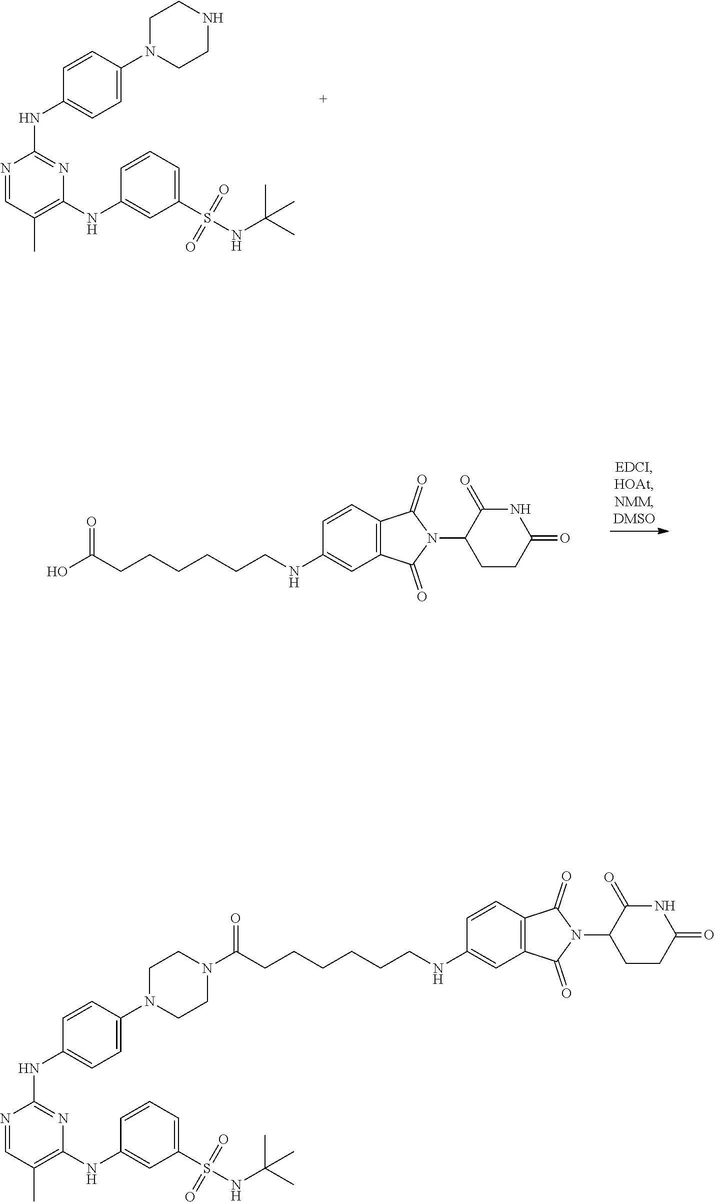 [SMILES: Cc1cnc(Nc2ccc(N3CCNCC3)cc2)nc1Nc1cccc(S(=O)(=O)NC(C)(C)C)c1 ; O=C(O)CCCCCCNc1ccc2c(c1)C(=O)N(C1CCC(=O)NC1=O)C2=O ; Cc1cnc(Nc2ccc(N3CCN(C(=O)CCCCCCNc4ccc5c(c4)C(=O)N(C4CCC(=O)NC4=O)C5=O)CC3)cc2)nc1Nc1cccc(S(=O)(=O)NC(C)(C)C)c1]

JA-230 was synthesized following the standard procedure for preparing JA-015 (9 mg, 54% yield). MS (ESI) m/z: 879.8 [M+H]⁺.

### Example 319. N-(tert-butyl)-3-((2-((4-(4-(8-((2-(2,6-dioxopiperidin-3-yl)-1,3-dioxoisoindolin-5-yl)amino)octanoyl)piperazin-1-yl)phenyl)amino)-5-methylpyrimidin-4-yl)amino)benzenesulfonamide (JA-231)

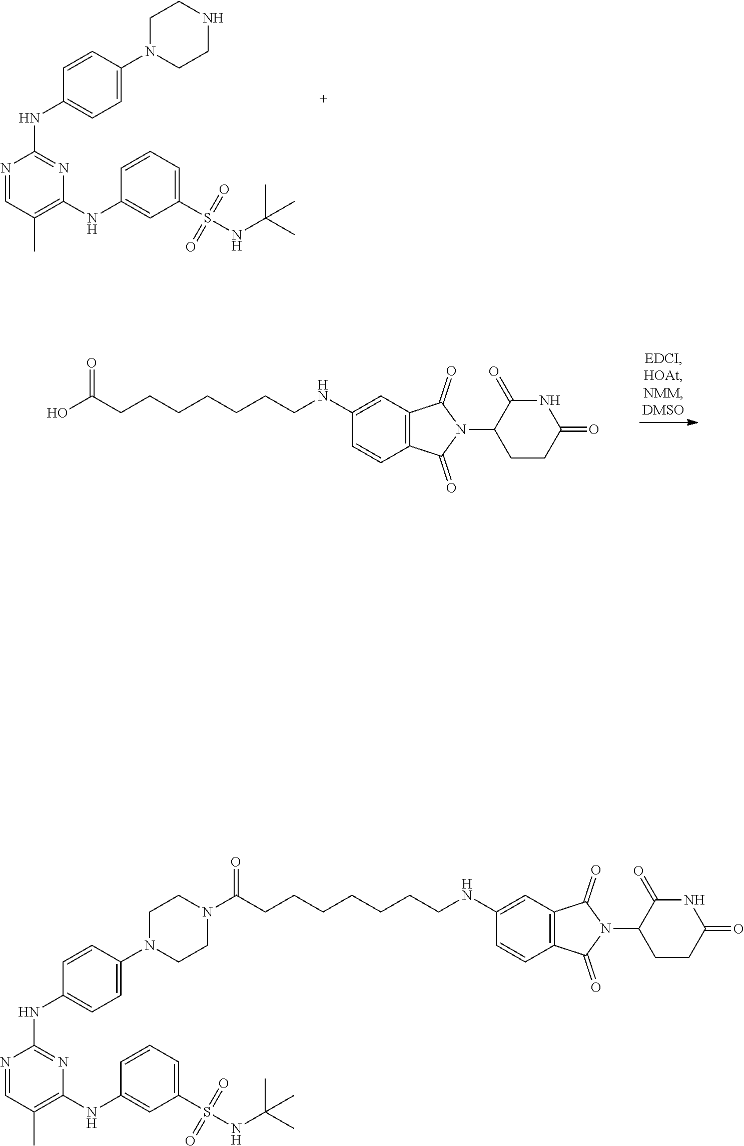 [SMILES: Cc1cnc(Nc2ccc(N3CCNCC3)cc2)nc1Nc1cccc(S(=O)(=O)NC(C)(C)C)c1 ; O=C(O)CCCCCCCNc1ccc2c(c1)C(=O)N(C1CCC(=O)NC1=O)C2=O ; Cc1cnc(Nc2ccc(N3CCN(C(=O)CCCCCCCNc4ccc5c(c4)C(=O)N(C4CCC(=O)NC4=O)C5=O)CC3)cc2)nc1Nc1cccc(S(=O)(=O)NC(C)(C)C)c1]

JA-231 was synthesized following the standard procedure for preparing JA-015 (9.6 mg, 57% yield). MS (ESI) m/z: 893.8 [M+H]⁺.

### Example 320. N-(tert-butyl)-3-((2-((4-(4-(3-(2-((2-(2,6-dioxopiperidin-3-yl)-1,3-dioxoisoindolin-5-yl)amino)ethoxy)propanoyl)piperazin-1-yl)phenyl)amino)-5-methylpyrimidin-4-yl)amino)benzenesulfonamide (JA-232)

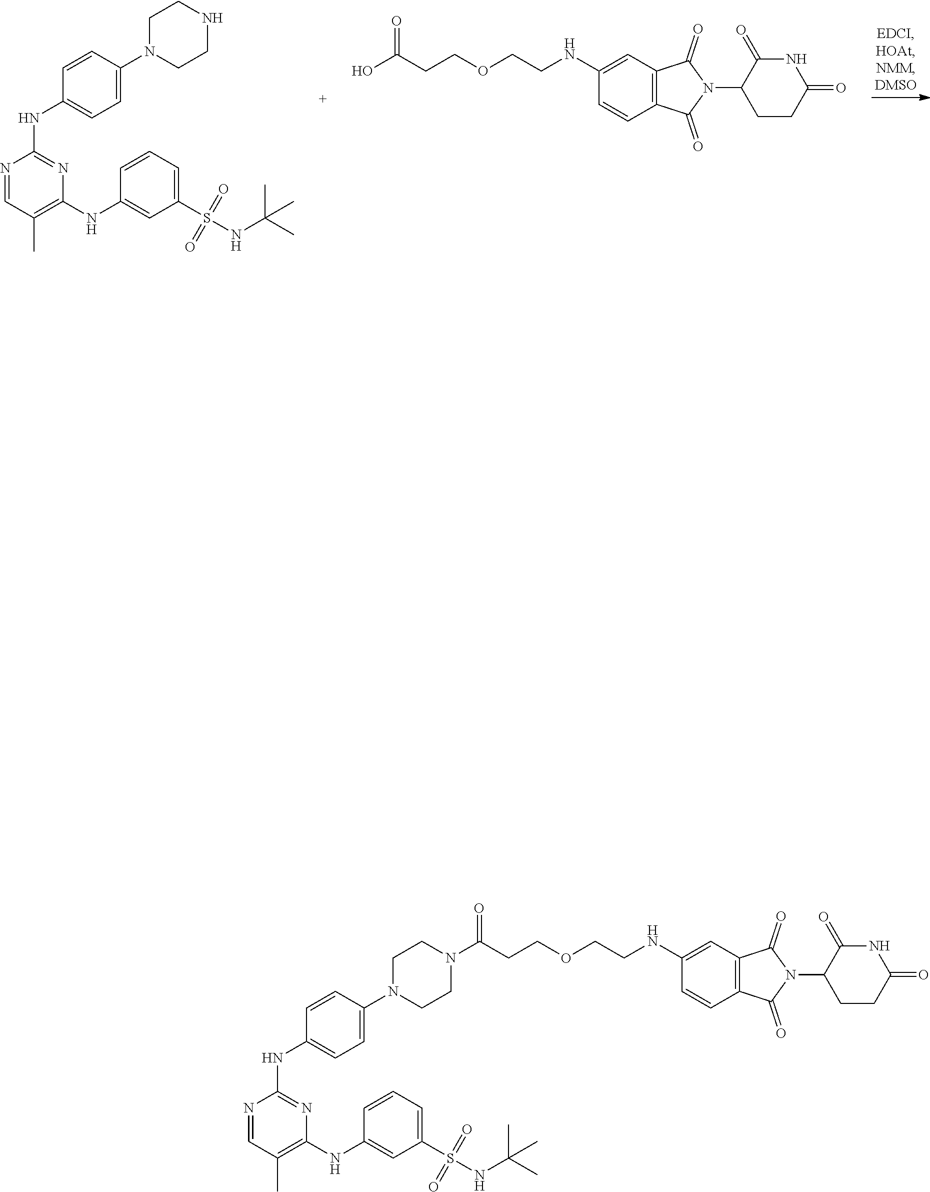 [SMILES: Cc1cnc(Nc2ccc(N3CCNCC3)cc2)nc1Nc1cccc(S(=O)(=O)NC(C)(C)C)c1 ; O=C(O)CCOCCNc1ccc2c(c1)C(=O)N(C1CCC(=O)NC1=O)C2=O ; Cc1cnc(Nc2ccc(N3CCN(C(=O)CCOCCNc4ccc5c(c4)C(=O)N(C4CCC(=O)NC4=O)C5=O)CC3)cc2)nc1Nc1cccc(S(=O)(=O)NC(C)(C)C)c1]

JA-232 was synthesized following the standard procedure for preparing JA-015 (9.9 mg, 61% yield). MS (ESI) m/z: 867.9 [M+H]⁺.

### Example 321. N-(tert-butyl)-3-((2-((4-(4-(3-(2-(2-((2-(2,6-dioxopiperidin-3-yl)-1,3-dioxoisoindolin-5-yl)amino)ethoxy)ethoxy)propanoyl)piperazin-1-yl)phenyl)amino)-5-methylpyrimidin-4-yl)amino)benzenesulfonamide (JA-233)

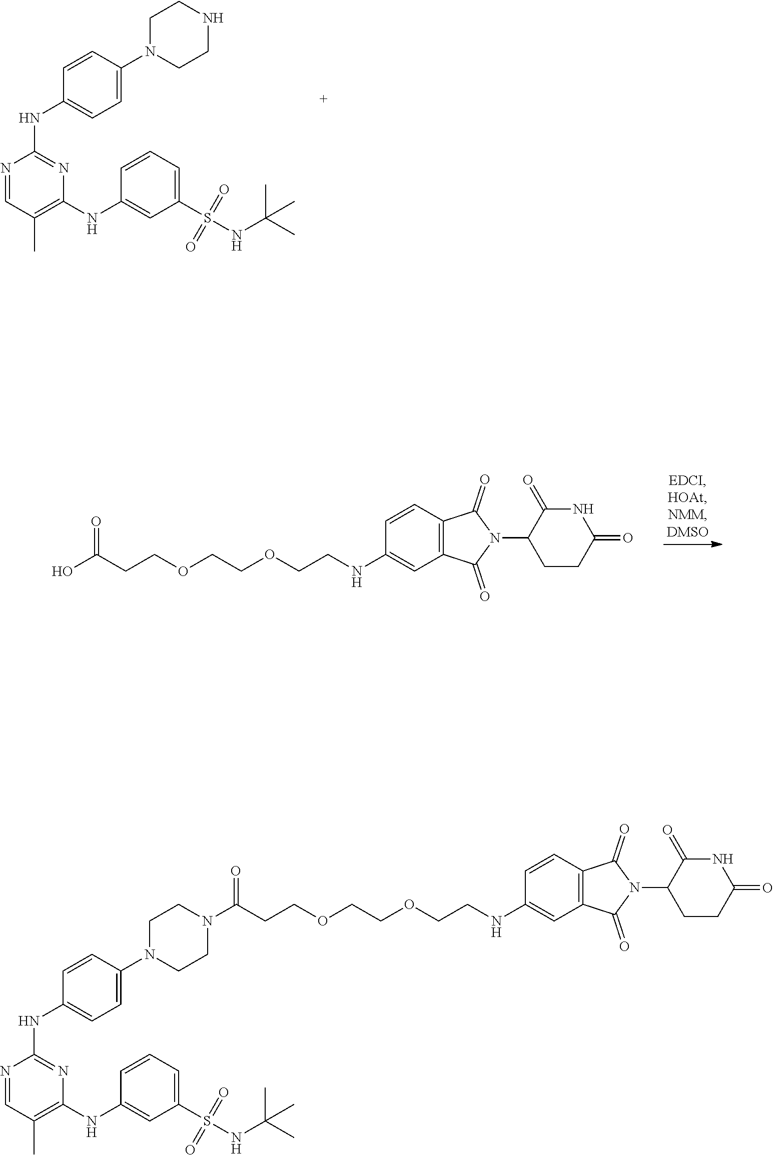 [SMILES: Cc1cnc(Nc2ccc(N3CCNCC3)cc2)nc1Nc1cccc(S(=O)(=O)NC(C)(C)C)c1 ; O=C(O)CCOCCOCCNc1ccc2c(c1)C(=O)N(C1CCC(=O)NC1=O)C2=O ; Cc1cnc(Nc2ccc(N3CCN(C(=O)CCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-233 was synthesized following the standard procedure for preparing JA-015 (10.2 mg, 60% yield). MS (ESI) m/z: 911.8 [M+H]⁺.

### Example 322. N-(tert-butyl)-3-((2-((4-(4-(3-(2-(2-(2-((2-(2,6-dioxopiperidin-3-yl)-1,3-dioxoisoindolin-5-yl)amino)ethoxy)ethoxy)ethoxy)propanoyl)piperazin-1-yl)phenyl)amino)-5-methylpyrimidin-4-yl)amino)benzenesulfonamide (JA-234)

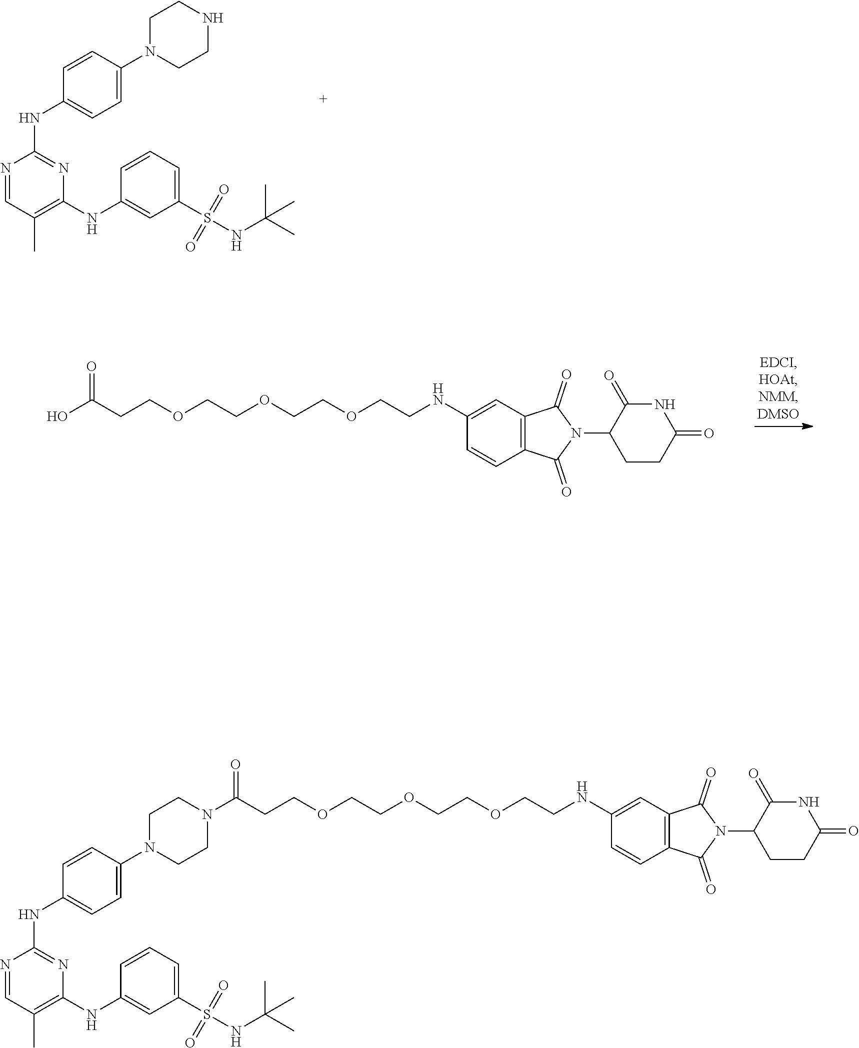 [SMILES: Cc1cnc(Nc2ccc(N3CCNCC3)cc2)nc1Nc1cccc(S(=O)(=O)NC(C)(C)C)c1 ; O=C(O)CCOCCOCCOCCNc1ccc2c(c1)C(=O)N(C1CCC(=O)NC1=O)C2=O ; Cc1cnc(Nc2ccc(N3CCN(C(=O)CCOCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-234 was synthesized following the standard procedure for preparing JA-015 (11 mg, 61% yield). MS (ESI) m/z: 955.8 [M+H]⁺.

### Example 323. N-(tert-butyl)-3-((2-((4-(4-(1-((2-(2,6-dioxopiperidin-3-yl)-1,3-dioxoisoindolin-5-yl)amino)-3,6,9,12-tetraoxapentadecan-15-oyl)piperazin-1-yl)phenyl)amino)-5-methylpyrimidin-4-yl)amino)benzenesulfonamide (JA-235)

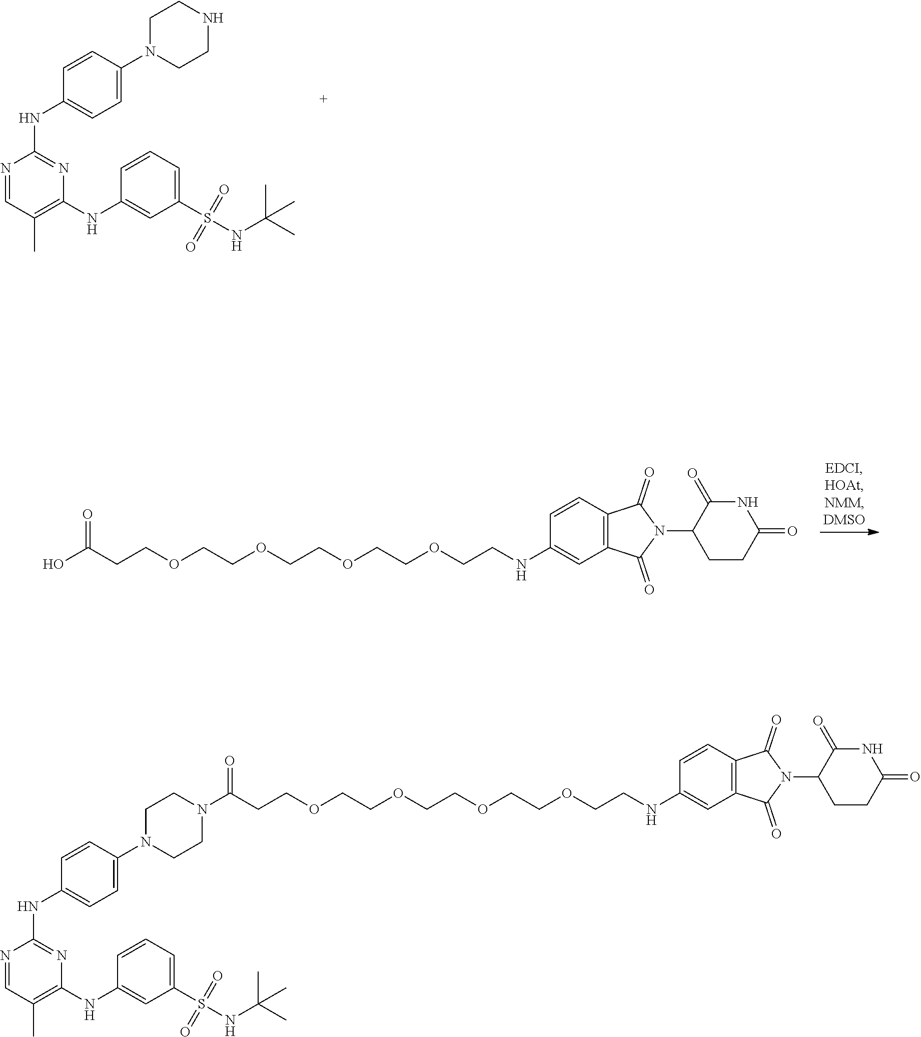 [SMILES: Cc1cnc(Nc2ccc(N3CCNCC3)cc2)nc1Nc1cccc(S(=O)(=O)NC(C)(C)C)c1 ; O=C(O)CCOCCOCCOCCOCCNc1ccc2c(c1)C(=O)N(C1CCC(=O)NC1=O)C2=O ; Cc1cnc(Nc2ccc(N3CCN(C(=O)CCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-235 was synthesized following the standard procedure for preparing JA-015 (11.5 mg, 61% yield). MS (ESI) m/z: 999.9 [M+H]⁺.

### Example 324. N-(tert-butyl)-3-((2-((4-(4-(1-((2-(2,6-dioxopiperidin-3-yl)-1,3-dioxoisoindolin-5-yl)amino)-3,6,9,12,15-pentaoxaoctadecan-18-oyl)piperazin-1-yl)phenyl)amino)-5-methylpyrimidin-4-yl)amino)benzenesulfonamide (JA-236)

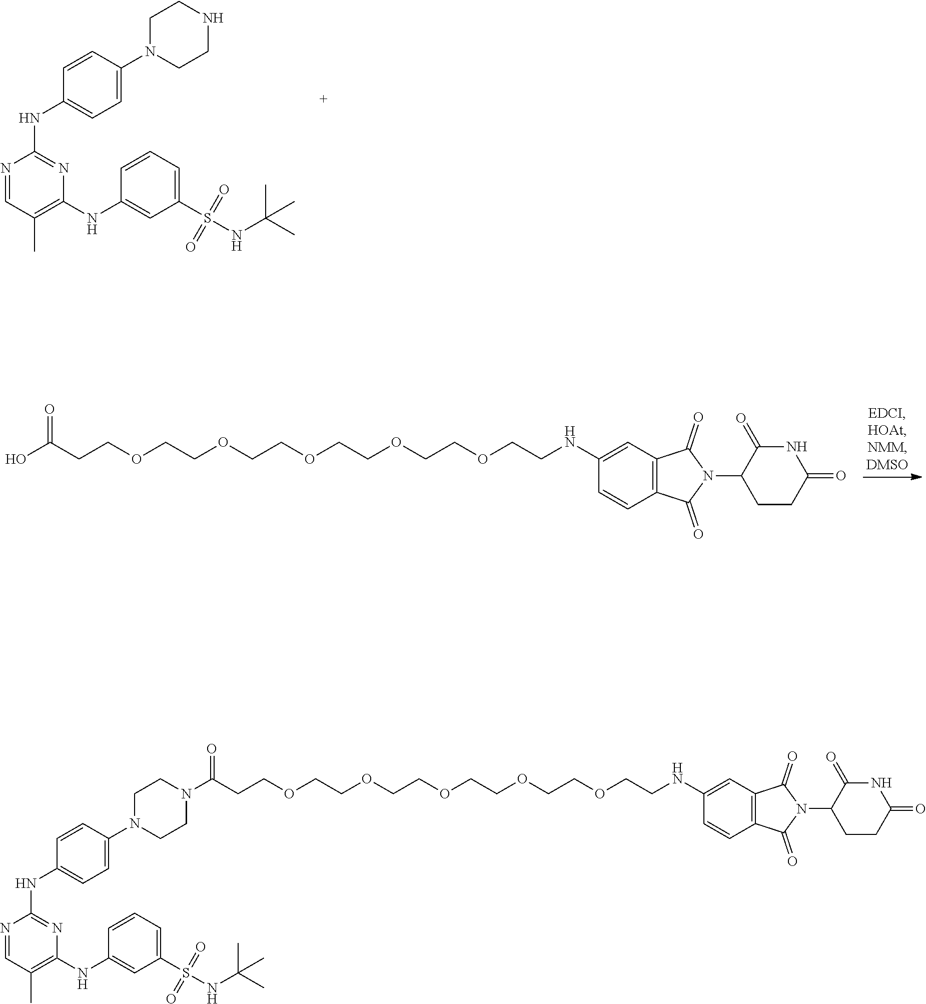 [SMILES: Cc1cnc(Nc2ccc(N3CCNCC3)cc2)nc1Nc1cccc(S(=O)(=O)NC(C)(C)C)c1 ; O=C(O)CCOCCOCCOCCOCCOCCNc1ccc2c(c1)C(=O)N(C1CCC(=O)NC1=O)C2=O ; Cc1cnc(Nc2ccc(N3CCN(C(=O)CCOCCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-236 was synthesized following the standard procedure for preparing JA-015 (10.5 mg, 54% yield). MS (ESI) m/z: 1043.9 [M+H]⁺.

### Example 325. 2-(4-(4-((4-((3-(N-(tert-butyl)sulfamoyl)phenyl)amino)-5-methylpyrimidin-2-yl)amino)phenyl)piperazin-1-yl)-N-(2-(2-((2-(2,6-dioxopiperidin-3-yl)-1,3-dioxoisoindolin-5-yl)amino)ethoxy)ethyl)acetamide (JA-237)

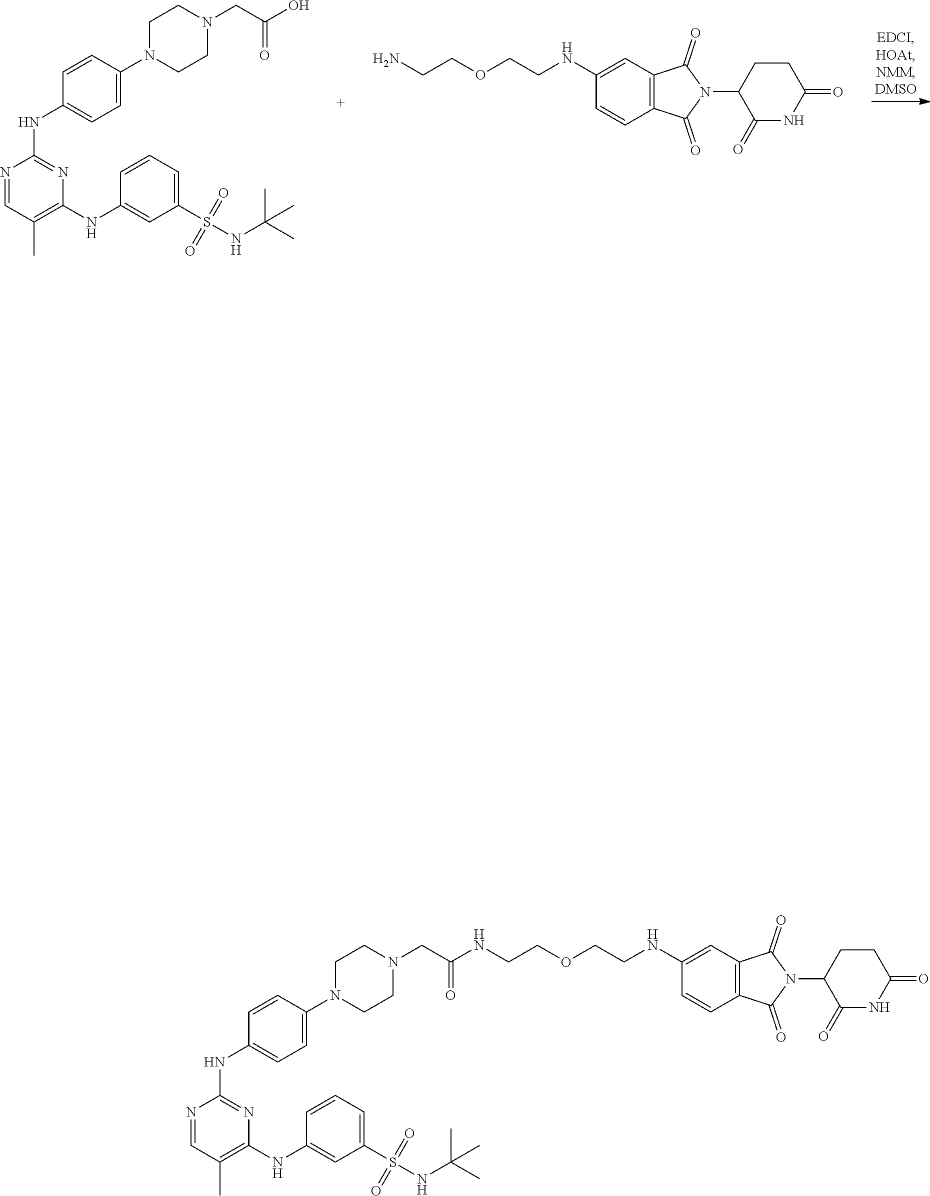 [SMILES: Cc1cnc(Nc2ccc(N3CCN(CC(=O)O)CC3)cc2)nc1Nc1cccc(S(=O)(=O)NC(C)(C)C)c1 ; NCCOCCNc1ccc2c(c1)C(=O)N(C1CCC(=O)NC1=O)C2=O ; Cc1cnc(Nc2ccc(N3CCN(CC(=O)NCCOCCNc4ccc5c(c4)C(=O)N(C4CCC(=O)NC4=O)C5=O)CC3)cc2)nc1Nc1cccc(S(=O)(=O)NC(C)(C)C)c1]

JA-237 was synthesized following the standard procedure for preparing JA-015 (7.6 mg, 47% yield). MS (ESI) m/z: 896.6 [M+H]⁺.

### Example 326. 2-(4-(4-((4-((3-(N-(tert-butyl)sulfamoyl)phenyl)amino)-5-methylpyrimidin-2-yl)amino)phenyl)piperazin-1-yl)-N-(2-(2-(2-((2-(2,6-dioxopiperidin-3-yl)-1,3-dioxoisoindolin-5-yl)amino)ethoxy)ethoxy)ethyl)acetamide (JA-238)

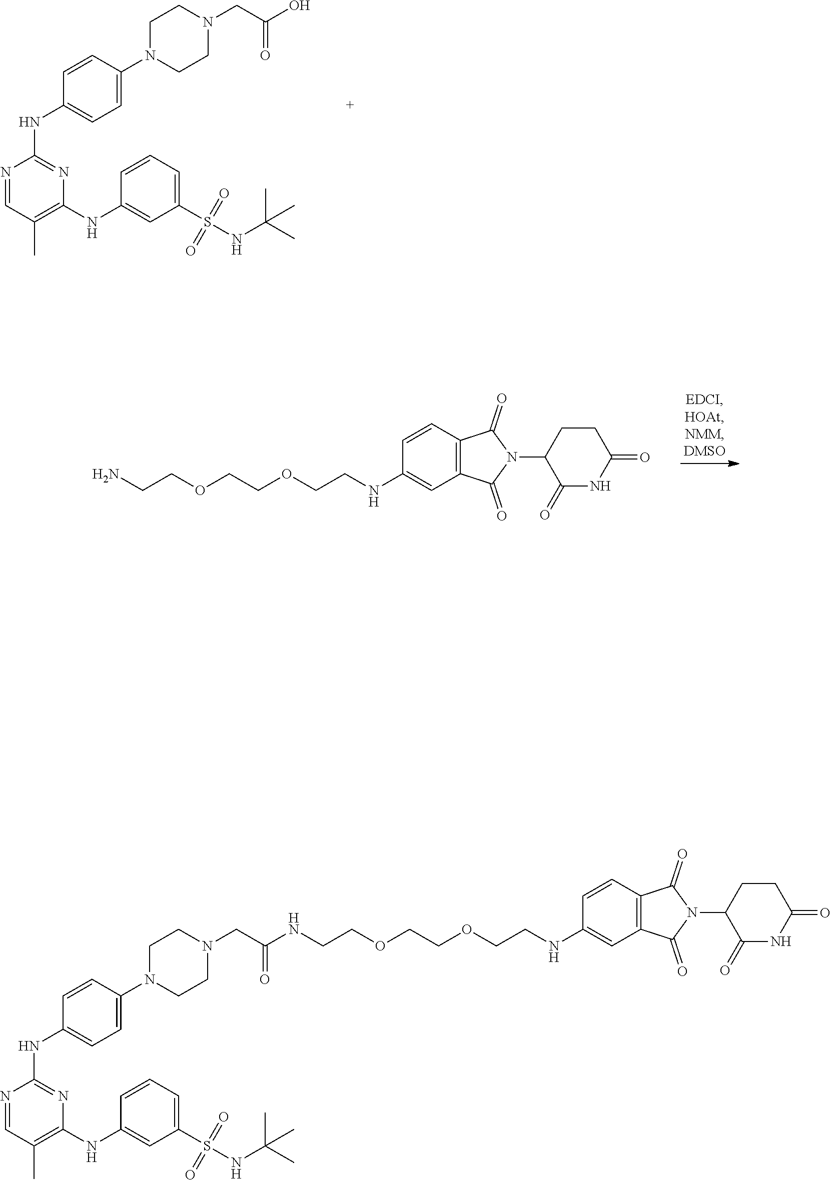 [SMILES: Cc1cnc(Nc2ccc(N3CCN(CC(=O)O)CC3)cc2)nc1Nc1cccc(S(=O)(=O)NC(C)(C)C)c1 ; NCCOCCOCCNc1ccc2c(c1)C(=O)N(C1CCC(=O)NC1=O)C2=O ; Cc1cnc(Nc2ccc(N3CCN(CC(=O)NCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-238 was synthesized following the standard procedure for preparing JA-015 (9.7 mg, 57% yield). MS (ESI) m/z: 940.8 [M+H]⁺.

### Example 327. 2-(4-(4-((4-((3-(N-(tert-butyl)sulfamoyl)phenyl)amino)-5-methylpyrimidin-2-yl)amino)phenyl)piperazin-1-yl)-N-(2-(2-(2-(2-((2-(2,6-dioxopiperidin-3-yl)-1,3-dioxoisoindolin-5-yl)amino)ethoxy)ethoxy)ethoxy)ethyl)acetamide (JA-239)

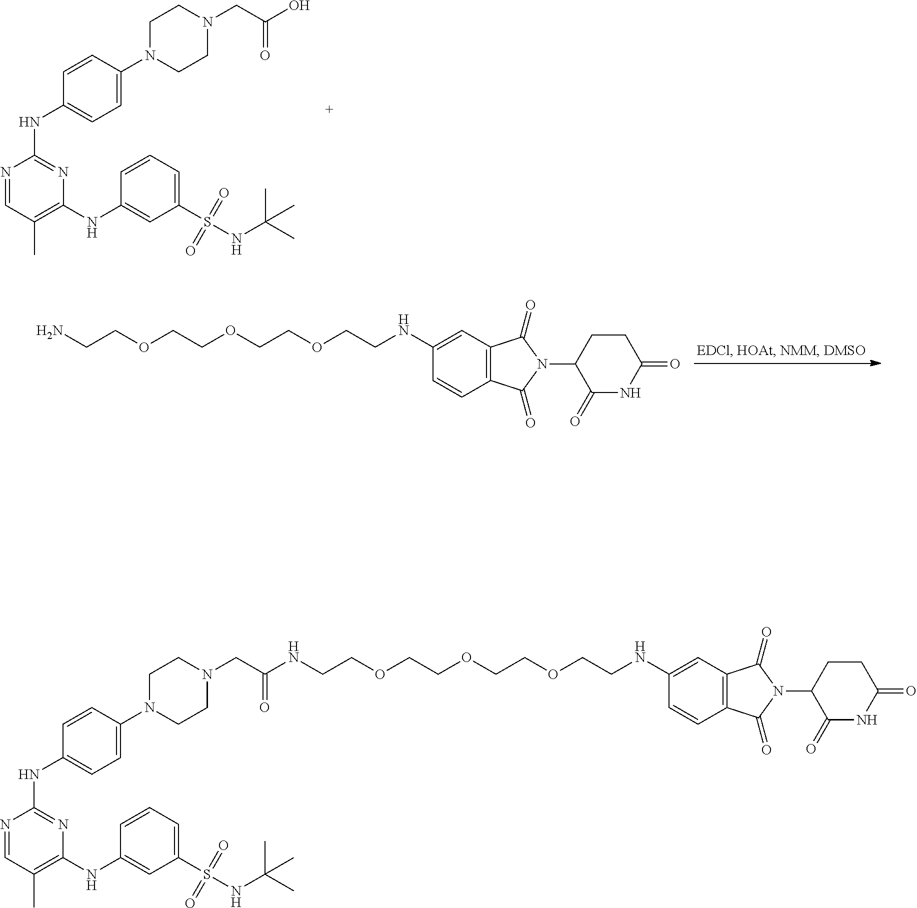 [SMILES: Cc1cnc(Nc2ccc(N3CCN(CC(=O)O)CC3)cc2)nc1Nc1cccc(S(=O)(=O)NC(C)(C)C)c1 ; NCCOCCOCCOCCNc1ccc2c(c1)C(=O)N(C1CCC(=O)NC1=O)C2=O ; Cc1cnc(Nc2ccc(N3CCN(CC(=O)NCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-239 was synthesized following the standard procedure for preparing JA-015 (9.2 mg, 52% yield). MS (ESI) m/z: 984.8 [M+H]⁺.

### Example 328. 2-(4-(4-((4-((3-(N-(tert-butyl)sulfamoyl)phenyl)amino)-5-methylpyrimidin-2-yl)amino)phenyl)piperazin-1-yl)-N-(14-((2-(2,6-dioxopiperidin-3-yl)-1,3-dioxoisoindolin-5-yl)amino)-3,6,9,12-tetraoxatetradecyl)acetamide (JA-240)

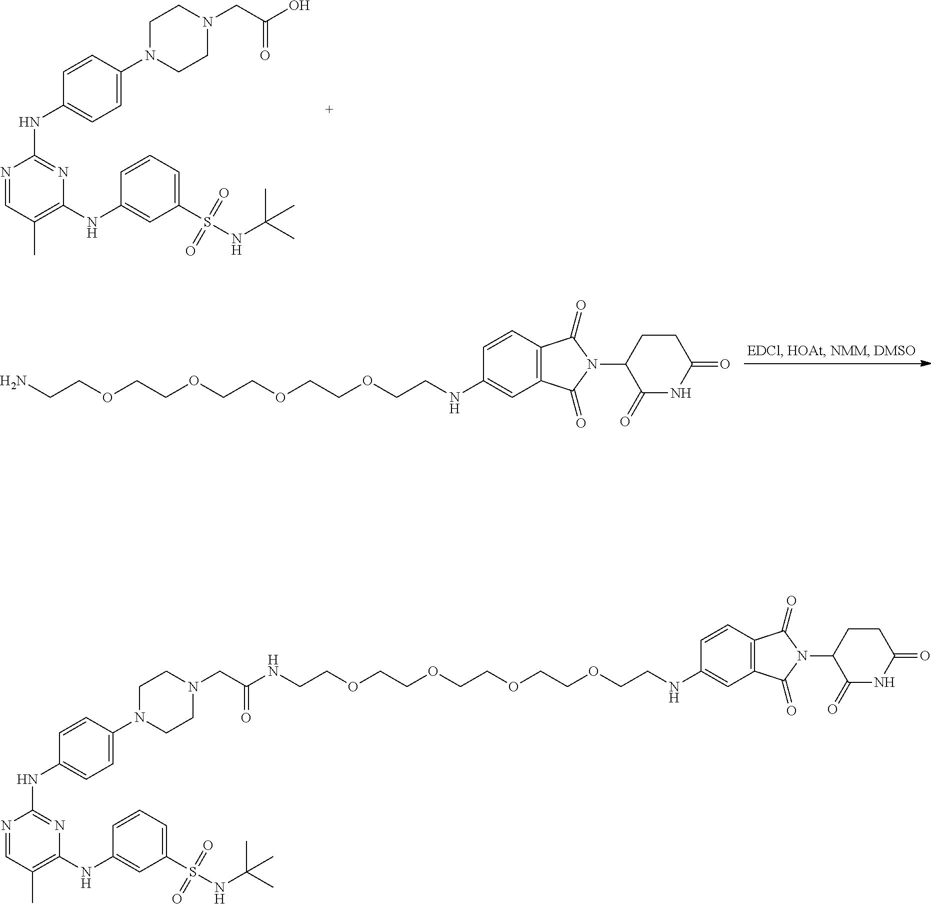 [SMILES: Cc1cnc(Nc2ccc(N3CCN(CC(=O)O)CC3)cc2)nc1Nc1cccc(S(=O)(=O)NC(C)(C)C)c1 ; NCCOCCOCCOCCOCCNc1ccc2c(c1)C(=O)N(C1CCC(=O)NC1=O)C2=O ; Cc1cnc(Nc2ccc(N3CCN(CC(=O)NCCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-240 was synthesized following the standard procedure for preparing JA-015 (9.2 mg, 50% yield). MS (ESI) m/z: 1028.8 [M+H]⁺.

### Example 329. 2-(4-(4-((4-((3-(N-(tert-butyl)sulfamoyl)phenyl)amino)-5-methylpyrimidin-2-yl)amino)phenyl)piperazin-1-yl)-N-(17-((2-(2,6-dioxopiperidin-3-yl)-1,3-dioxoisoindolin-5-yl)amino)-3,6,9,12,15-pentaoxaheptadecyl)acetamide (JA-241)

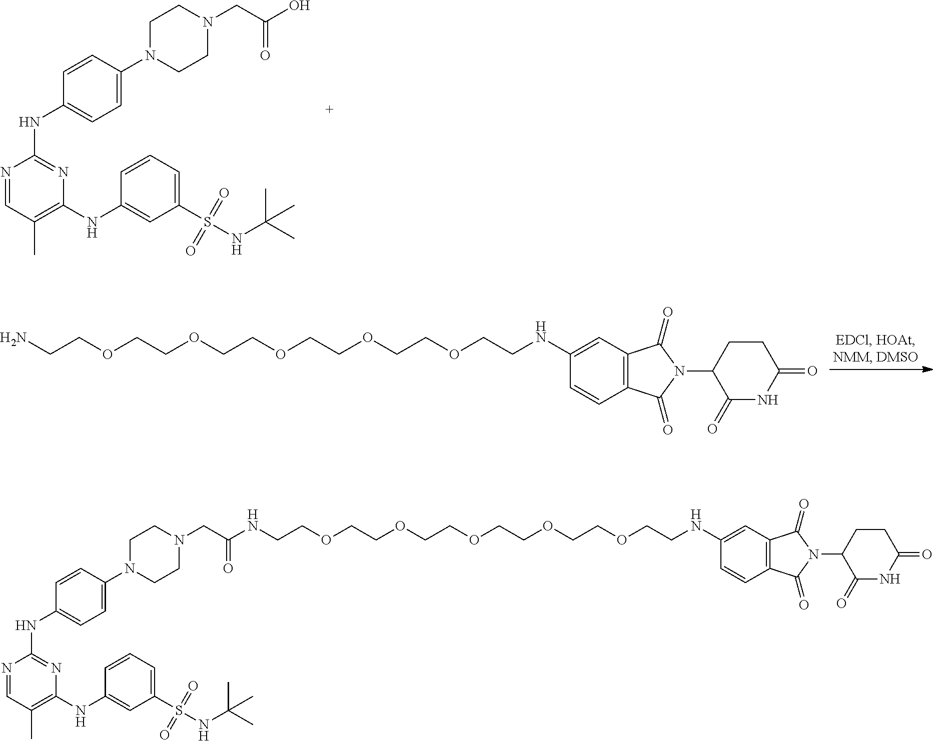 [SMILES: Cc1cnc(Nc2ccc(N3CCN(CC(=O)O)CC3)cc2)nc1Nc1cccc(S(=O)(=O)NC(C)(C)C)c1 ; NCCOCCOCCOCCOCCOCCNc1ccc2c(c1)C(=O)N(C1CCC(=O)NC1=O)C2=O ; Cc1cnc(Nc2ccc(N3CCN(CC(=O)NCCOCCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1]

JA-241 was synthesized following the standard procedure for preparing JA-015 (9.2 mg, 48% yield). MS (ESI) m/z: 1072.9 [M+H]⁺.

### Example 330. 2-(4-(4-((4-((3-(N-(tert-butyl)sulfamoyl)phenyl)amino)-5-methylpyrimidin-2-yl)amino)phenyl)piperazin-1-yl)-N-(2-((2-(2,6-dioxopiperidin-3-yl)-1,3-dioxoisoindolin-5-yl)amino)ethyl)acetamide (JA-242)

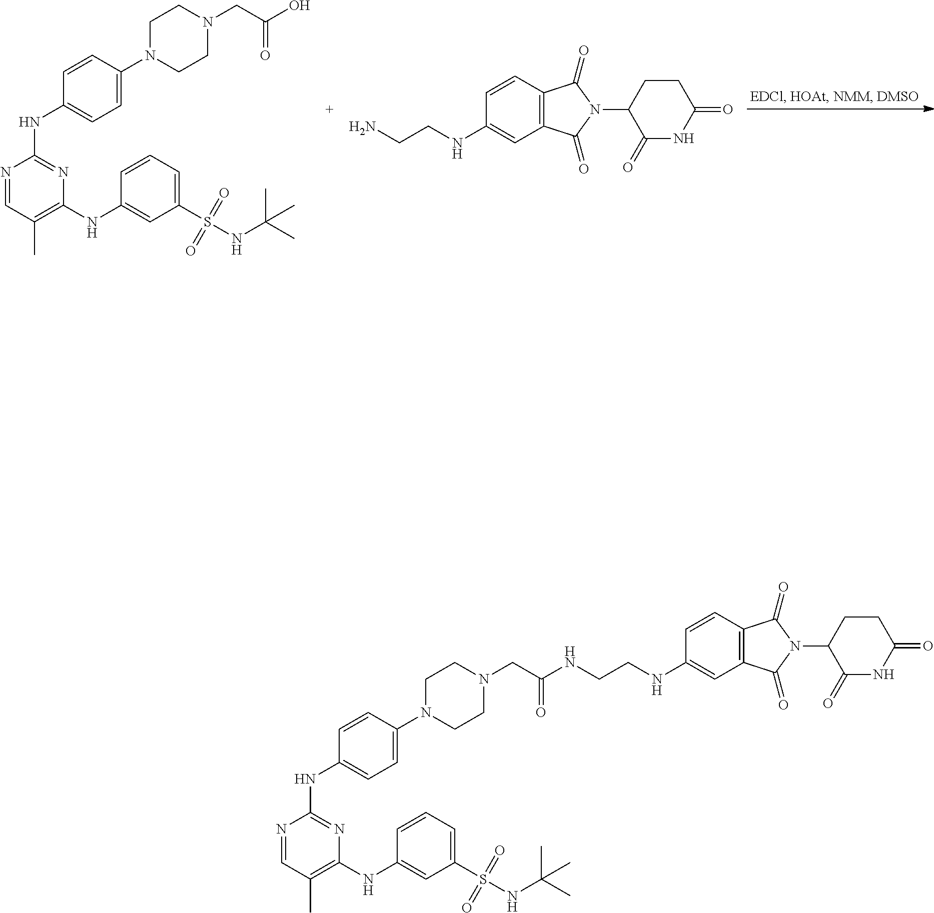 [SMILES: Cc1cnc(Nc2ccc(N3CCN(CC(=O)O)CC3)cc2)nc1Nc1cccc(S(=O)(=O)NC(C)(C)C)c1 ; NCCNc1ccc2c(c1)C(=O)N(C1CCC(=O)NC1=O)C2=O ; Cc1cnc(Nc2ccc(N3CCN(CC(=O)NCCNc4ccc5c(c4)C(=O)N(C4CCC(=O)NC4=O)C5=O)CC3)cc2)nc1Nc1cccc(S(=O)(=O)NC(C)(C)C)c1]

JA-242 was synthesized following the standard procedure for preparing JA-015 (9.2 mg, 60% yield). MS (ESI) m/z: 852.6 [M+H]⁺.

### Example 331. 2-(4-(4-((4-((3-(N-(tert-butyl)sulfamoyl)phenyl)amino)-5-methylpyrimidin-2-yl)amino)phenyl)piperazin-1-yl)-N-(4-((2-(2,6-dioxopiperidin-3-yl)-1,3-dioxoisoindolin-5-yl)amino)butyl)acetamide (JA-243)

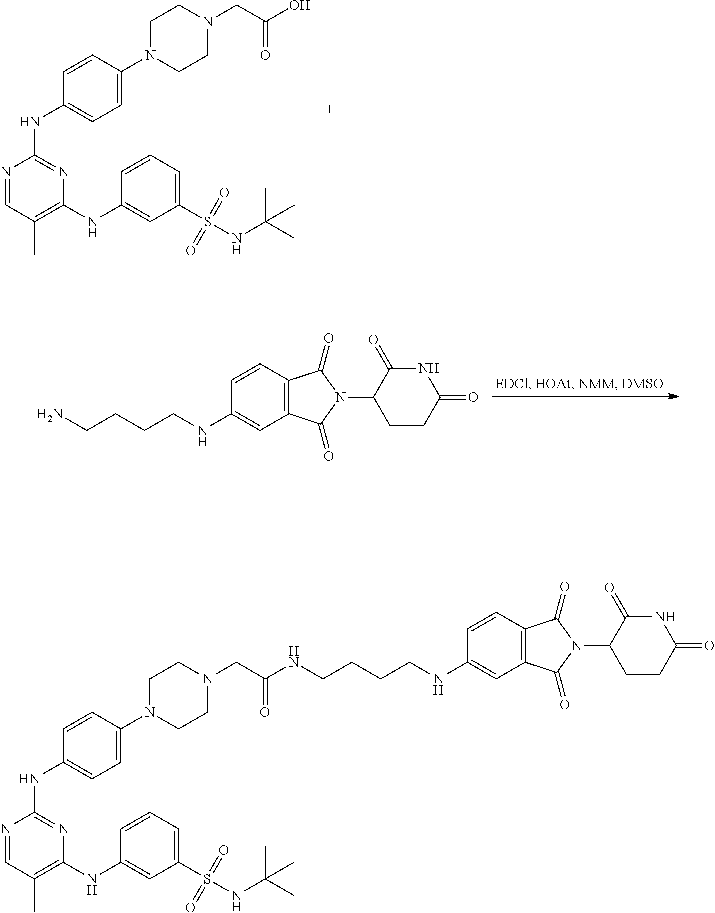 [SMILES: Cc1cnc(Nc2ccc(N3CCN(CC(=O)O)CC3)cc2)nc1Nc1cccc(S(=O)(=O)NC(C)(C)C)c1 ; NCCCCNc1ccc2c(c1)C(=O)N(C1CCC(=O)NC1=O)C2=O ; Cc1cnc(Nc2ccc(N3CCN(CC(=O)NCCCCNc4ccc5c(c4)C(=O)N(C4CCC(=O)NC4=O)C5=O)CC3)cc2)nc1Nc1cccc(S(=O)(=O)NC(C)(C)C)c1]

JA-243 was synthesized following the standard procedure for preparing JA-015 (8.8 mg, 55% yield). MS (ESI) m/z: 880.7 [M+H]⁺.

### Example 332. 2-(4-(4-((4-((3-(N-(tert-butyl)sulfamoyl)phenyl)amino)-5-methylpyrimidin-2-yl)amino)phenyl)piperazin-1-yl)-N-(5-((2-(2,6-dioxopiperidin-3-yl)-1,3-dioxoisoindolin-5-yl)amino)pentyl)acetamide (JA-244)

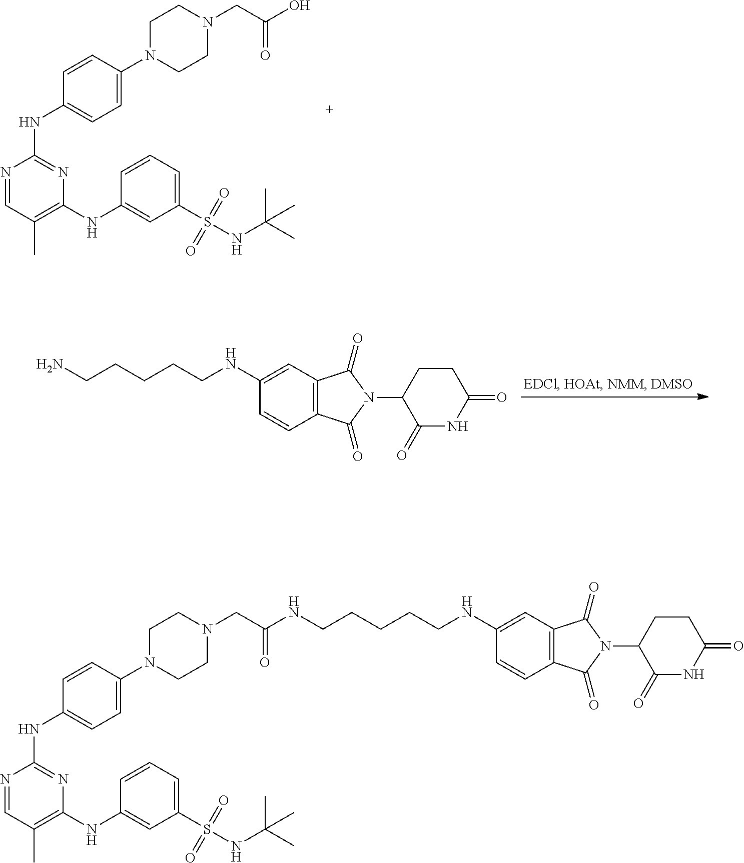 [SMILES: Cc1cnc(Nc2ccc(N3CCN(CC(=O)O)CC3)cc2)nc1Nc1cccc(S(=O)(=O)NC(C)(C)C)c1 ; NCCCCCNc1ccc2c(c1)C(=O)N(C1CCC(=O)NC1=O)C2=O ; Cc1cnc(Nc2ccc(N3CCN(CC(=O)NCCCCCNc4ccc5c(c4)C(=O)N(C4CCC(=O)NC4=O)C5=O)CC3)cc2)nc1Nc1cccc(S(=O)(=O)NC(C)(C)C)c1]

JA-244 was synthesized following the standard procedure for preparing JA-015 (9.3 mg, 58% yield). MS (ESI) m/z: 894.6 [M+H]⁺.

### Example 333. 2-(4-(4-((4-((3-(N-(tert-butyl)sulfamoyl)phenyl)amino)-5-methylpyrimidin-2-yl)amino)phenyl)piperazin-1-yl)-N-(6-((2-(2,6-dioxopiperidin-3-yl)-1,3-dioxoisoindolin-5-yl)amino)hexyl)acetamide (JA-245)

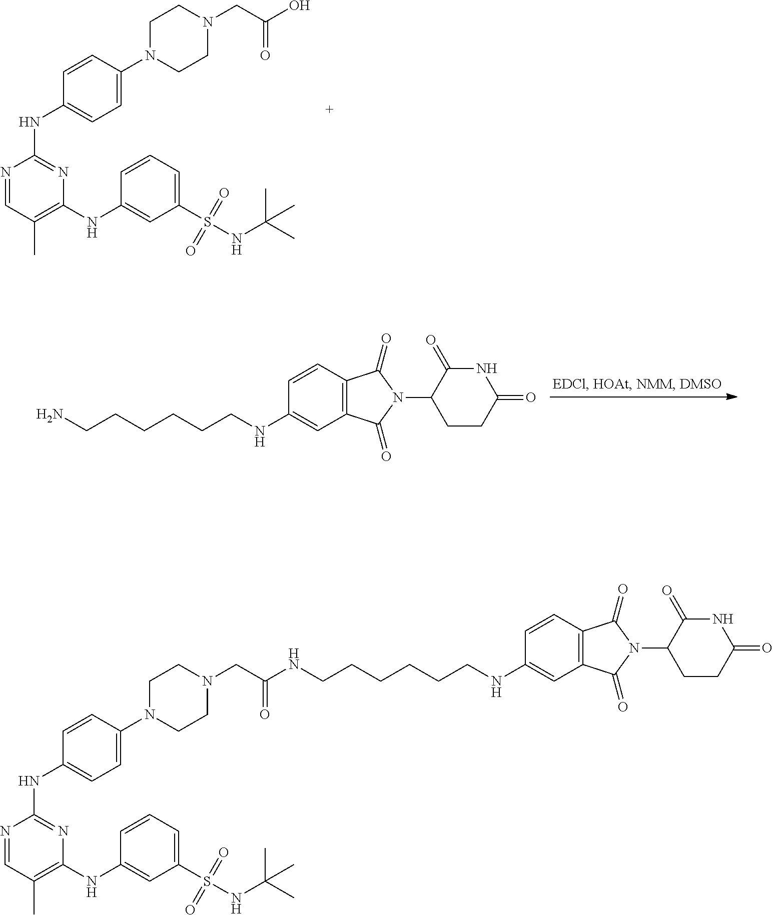 [SMILES: Cc1cnc(Nc2ccc(N3CCN(CC(=O)O)CC3)cc2)nc1Nc1cccc(S(=O)(=O)NC(C)(C)C)c1 ; NCCCCCCNc1ccc2c(c1)C(=O)N(C1CCC(=O)NC1=O)C2=O ; Cc1cnc(Nc2ccc(N3CCN(CC(=O)NCCCCCCNc4ccc5c(c4)C(=O)N(C4CCC(=O)NC4=O)C5=O)CC3)cc2)nc1Nc1cccc(S(=O)(=O)NC(C)(C)C)c1]

JA-245 was synthesized following the standard procedure for preparing JA-015 (11 mg, 67% yield). MS (ESI) m/z: 908.6 [M+H]⁺.

### Example 334. 2-(4-(4-((4-((3-(N-(tert-butyl)sulfamoyl)phenyl)amino)-5-methylpyrimidin-2-yl)amino)phenyl)piperazin-1-yl)-N-(7-((2-(2,6-dioxopiperidin-3-yl)-1,3-dioxoisoindolin-5-yl)amino)heptyl)acetamide (JA-246)

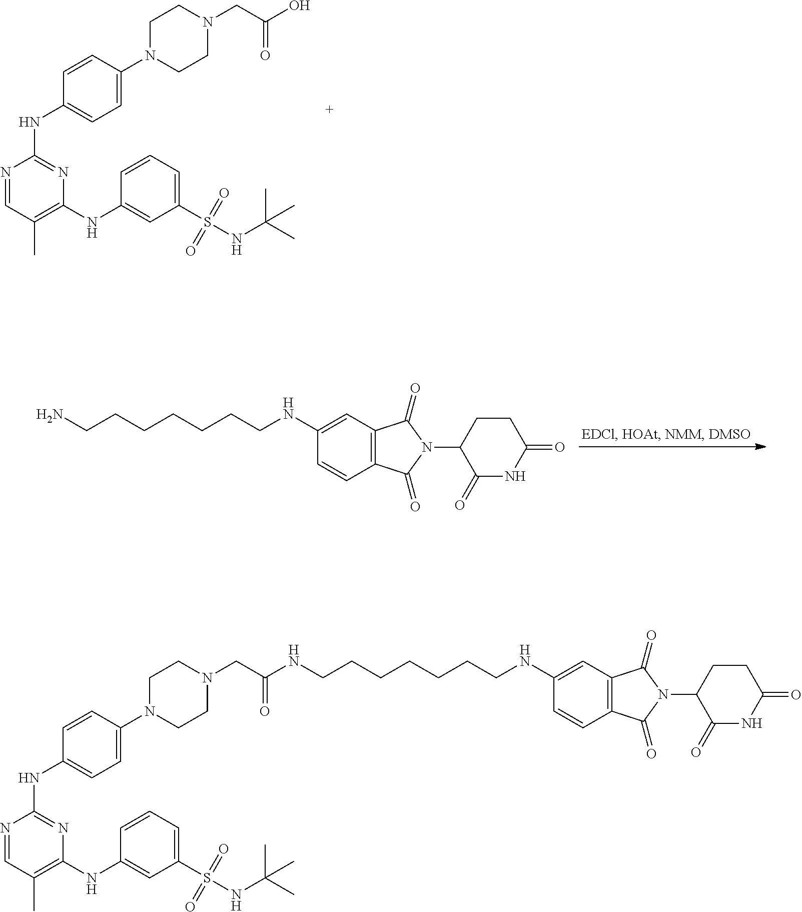 [SMILES: Cc1cnc(Nc2ccc(N3CCN(CC(=O)O)CC3)cc2)nc1Nc1cccc(S(=O)(=O)NC(C)(C)C)c1 ; NCCCCCCCNc1ccc2c(c1)C(=O)N(C1CCC(=O)NC1=O)C2=O ; Cc1cnc(Nc2ccc(N3CCN(CC(=O)NCCCCCCCNc4ccc5c(c4)C(=O)N(C4CCC(=O)NC4=O)C5=O)CC3)cc2)nc1Nc1cccc(S(=O)(=O)NC(C)(C)C)c1]

JA-246 was synthesized following the standard procedure for preparing JA-015 (9.5 mg, 57% yield). MS (ESI) m/z: 922.7 [M+H]⁺.

### Example 335. 2-(4-(4-((4-((3-(N-(tert-butyl)sulfamoyl)phenyl)amino)-5-methylpyrimidin-2-yl)amino)phenyl)piperazin-1-yl)-N-(8-((2-(2,6-dioxopiperidin-3-yl)-1,3-dioxoisoindolin-5-yl)amino)octyl)acetamide (JA-247)

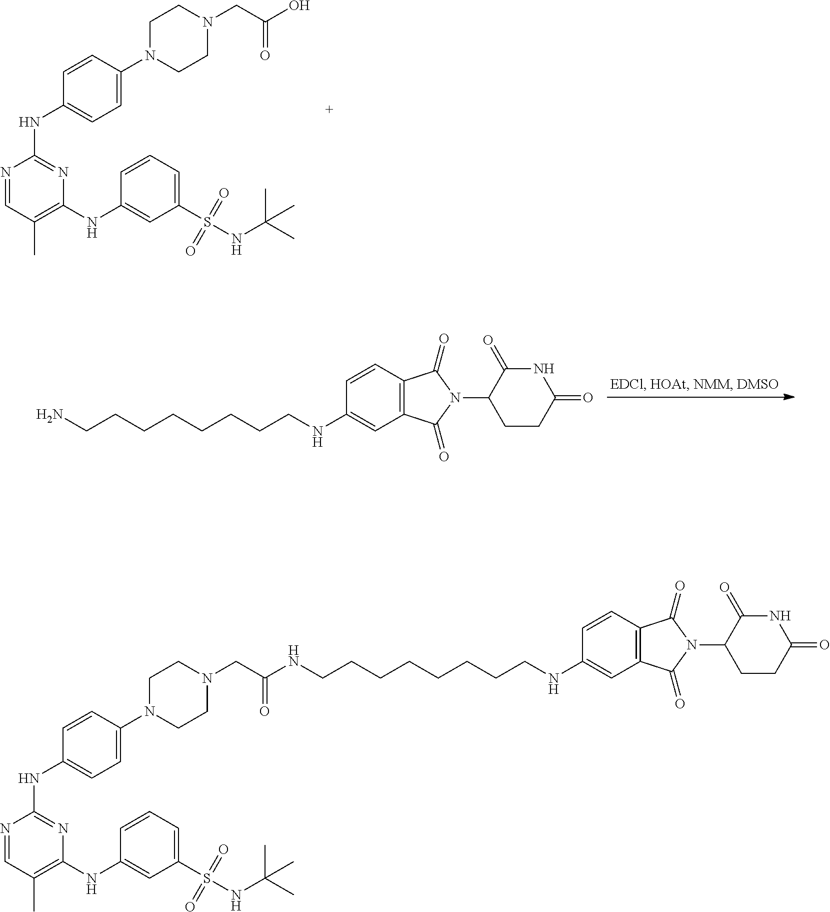 [SMILES: Cc1cnc(Nc2ccc(N3CCN(CC(=O)O)CC3)cc2)nc1Nc1cccc(S(=O)(=O)NC(C)(C)C)c1 ; NCCCCCCCCNc1ccc2c(c1)C(=O)N(C1CCC(=O)NC1=O)C2=O ; Cc1cnc(Nc2ccc(N3CCN(CC(=O)NCCCCCCCCNc4ccc5c(c4)C(=O)N(C4CCC(=O)NC4=O)C5=O)CC3)cc2)nc1Nc1cccc(S(=O)(=O)NC(C)(C)C)c1]

JA-247 was synthesized following the standard procedure for preparing JA-015 (7.8 mg, 46% yield). MS (ESI) m/z: 936.7 [M+H]⁺.

### Example 336. 2-(2,6-Dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)-7-oxohept-1-yn-1-yl)isoindoline-1,3-dione (JA-248)

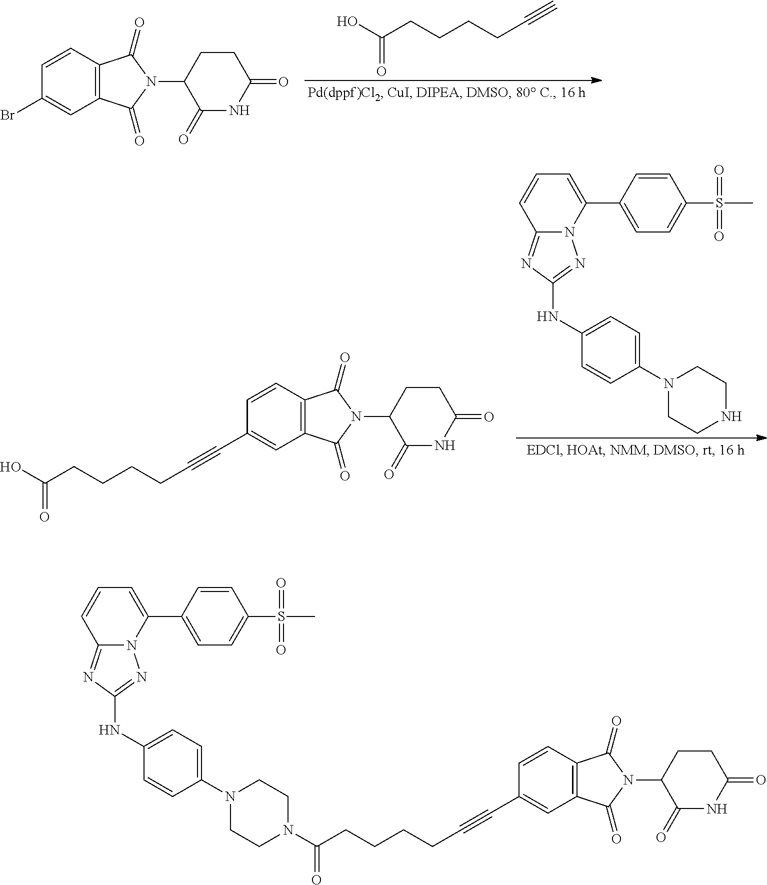

### Step 1: Synthesis of 7-(2-(2,6-dioxopiperidin-3-yl)-1,3-dioxoisoindolin-5-yl)hept-6-ynoic acid

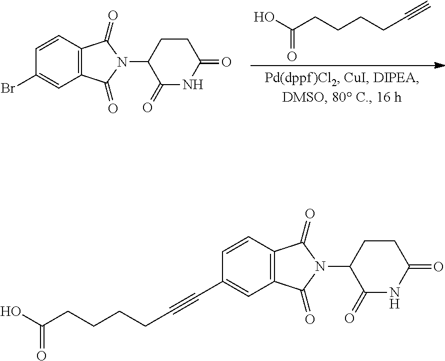

A mixture of 5-bromo-2-(2,6-dioxopiperidin-3-yl)isoindoline-1,3-dione (100 mg, 298 umol), Pd(dppf)Cl₂ (22 mg, 29.8 umol), hept-6-ynoic acid (56 mg, 447 umol), CuI (6 mg, 29.8 umol) and DIPEA (116 mg, 894 umol) in DMSO (6 mL) was stirred at 80° C. for 16 h. The reaction mixture was purified by reverse-phase chromatography to give the desired product (45 mg, 39% yield) as a light yellow solid. MS (ESI) m/z: 381.1 [M−H]⁻.

### Step 2: Synthesis of 2-(2,6-dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)-7-oxohept-1-yn-1-yl)isoindoline-1,3-dione

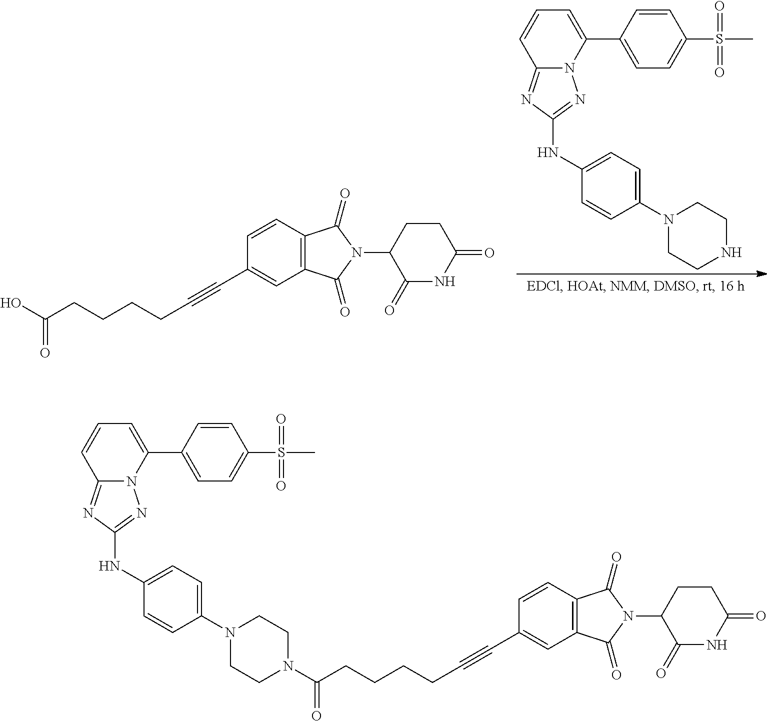 [SMILES: O=C(O)CCCCC#Cc1ccc2c(c1)C(=O)N(C1CCC(=O)NC1=O)C2=O ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCC#Cc6ccc7c(c6)C(=O)N(C6CCC(=O)NC6=O)C7=O)CC5)cc4)nn23)cc1]

JA-248 was synthesized following the standard procedure for preparing JA-001 (8 mg, 38% yield) as a light yellow solid. MS (ESI) m/z: 813.3 [M+H]⁺.

### Example 337. 2-(2,6-Dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)-7-oxoheptyl)isoindoline-1,3-dione (JA-249)

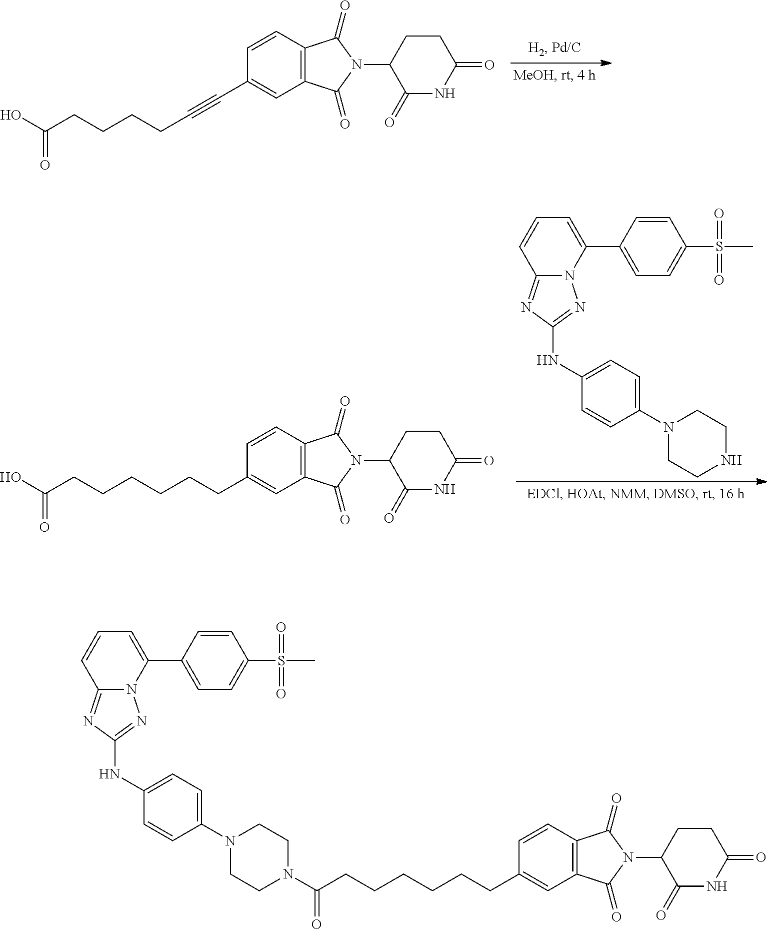 [SMILES: O=C(O)CCCCC#Cc1ccc2c(c1)C(=O)N(C1CCC(=O)NC1=O)C2=O ; O=C(O)CCCCCCc1ccc2c(c1)C(=O)N(C1CCC(=O)NC1=O)C2=O ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCCc6ccc7c(c6)C(=O)N(C6CCC(=O)NC6=O)C7=O)CC5)cc4)nn23)cc1]

### Step 1: Synthesis of 7-(2-(2,6-dioxopiperidin-3-yl)-1,3-dioxoisoindolin-5-yl)heptanoic acid

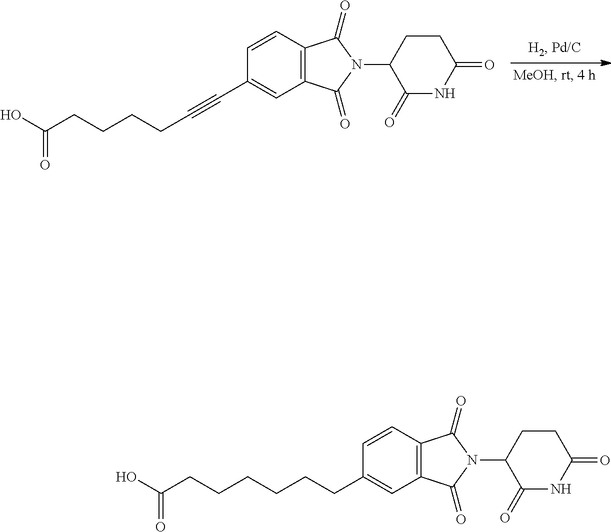 [SMILES: O=C(O)CCCCC#Cc1ccc2c(c1)C(=O)N(C1CCC(=O)NC1=O)C2=O ; O=C(O)CCCCCCc1ccc2c(c1)C(=O)N(C1CCC(=O)NC1=O)C2=O]

To a solution of 7-(2-(2,6-dioxopiperidin-3-yl)-1,3-dioxoisoindolin-5-yl)hept-6-ynoic acid (100 mg, 261 umol) in MeOH (10 mL) was added Pd/C (5.5 mg, 2.6 umol). The mixture was stirred at 25° C. for 16 h under hydrogen atmosphere. The reaction mixture was purified by reverse-phase chromatography to give the desired product (83 mg, 82% yield) as a light yellow solid. MS (ESI) m/z: 385.1 [M−H]⁻.

### Step 2: Synthesis of 2-(2,6-dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)-7-oxoheptyl)isoindoline-1,3-dione

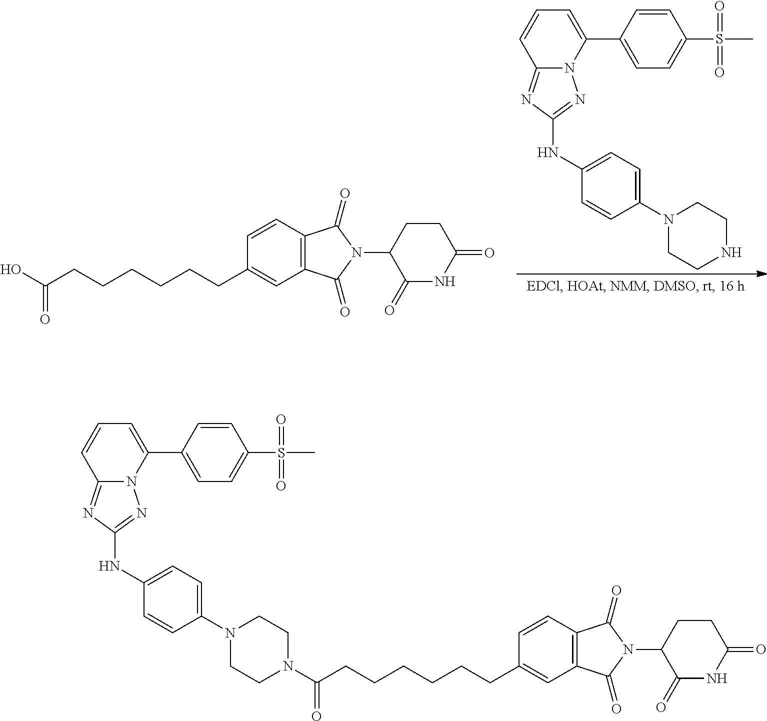 [SMILES: O=C(O)CCCCCCc1ccc2c(c1)C(=O)N(C1CCC(=O)NC1=O)C2=O ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCCc6ccc7c(c6)C(=O)N(C6CCC(=O)NC6=O)C7=O)CC5)cc4)nn23)cc1]

JA-249 was synthesized following the standard procedure for preparing JA-001 (10.2 mg, 40% yield) as a light yellow solid. MS (ESI) m/z: 817.3 [M+H]⁺.

### Example 338. 3-(5-(7-(4-(4-((5-(4-(Methylsulfonyl)phenyl)-[1,2,4]triazolo[1,5-a]pyridin-2-yl)amino)phenyl)piperazin-1-yl)-7-oxohept-1-yn-1-yl)-1-oxoisoindolin-2-yl)piperidine-2,6-dione (JA-250)

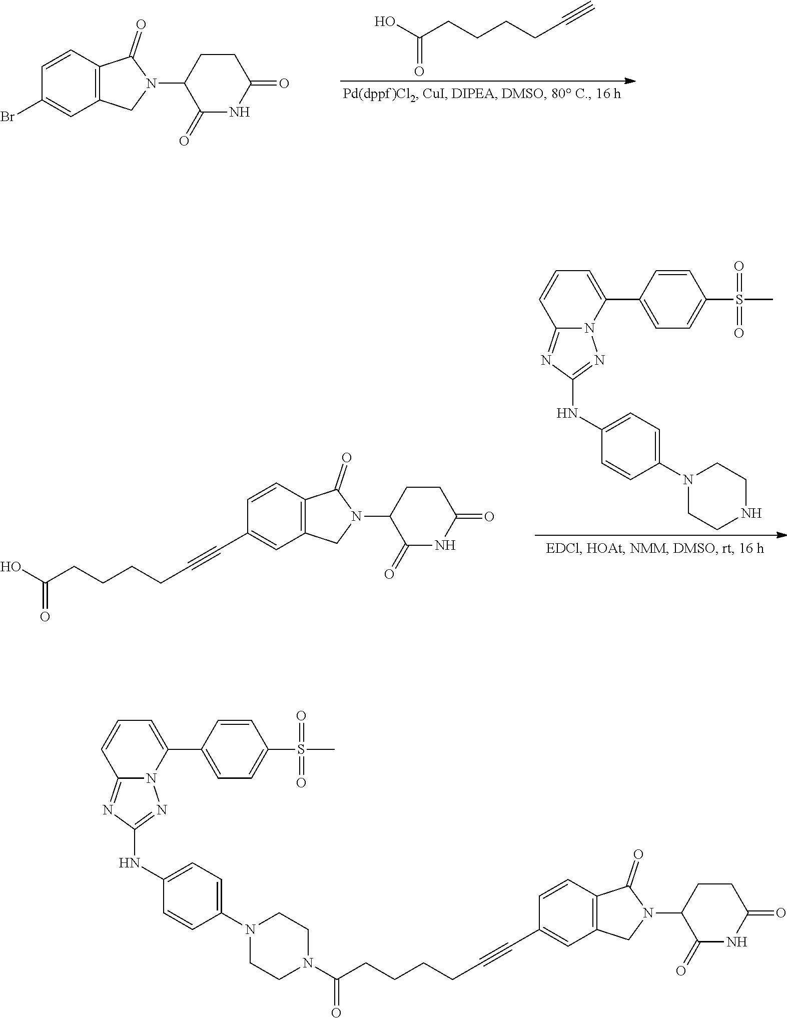

### Step 1: Synthesis of 7-(2-(2,6-dioxopiperidin-3-yl)-1-oxoisoindolin-5-yl)hept-6-ynoic acid

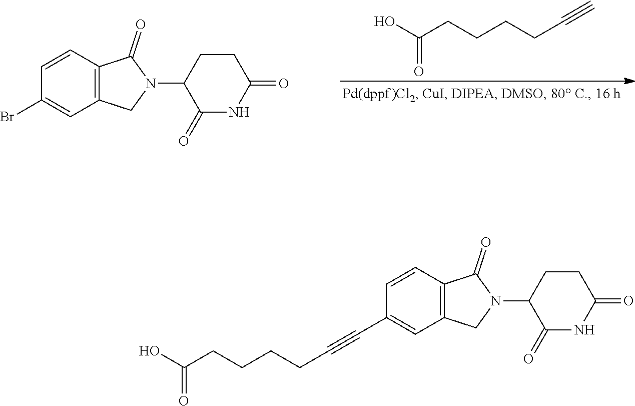

A mixture of 3-(5-bromo-1-oxoisoindolin-2-yl)piperidine-2,6-dione (100 mg, 311 umol), Pd(dppf)Cl₂ (23 mg, 31.1 umol), hept-6-ynoic acid (59 mg, 467 umol), CuI (6 mg, 31.1 umol) and DIPEA (120 mg, 931 umol) in DMSO (6 mL) was stirred at 80° C. for 16 h. The reaction mixture was purified by reverse-phase chromatography to give the desired product (51 mg, 45% yield) as a light yellow solid. MS (ESI) m/z: 367.1 [M−H]⁻.

### Step 2: Synthesis of 3-(5-(7-(4-(4-((5-(4-(methylsulfonyl)phenyl)-[1,2,4]triazolo[1,5-a]pyridin-2-yl)amino)phenyl)piperazin-1-yl)-7-oxohept-1-yn-1-yl)-1-oxoisoindolin-2-yl)piperidine-2,6-dione

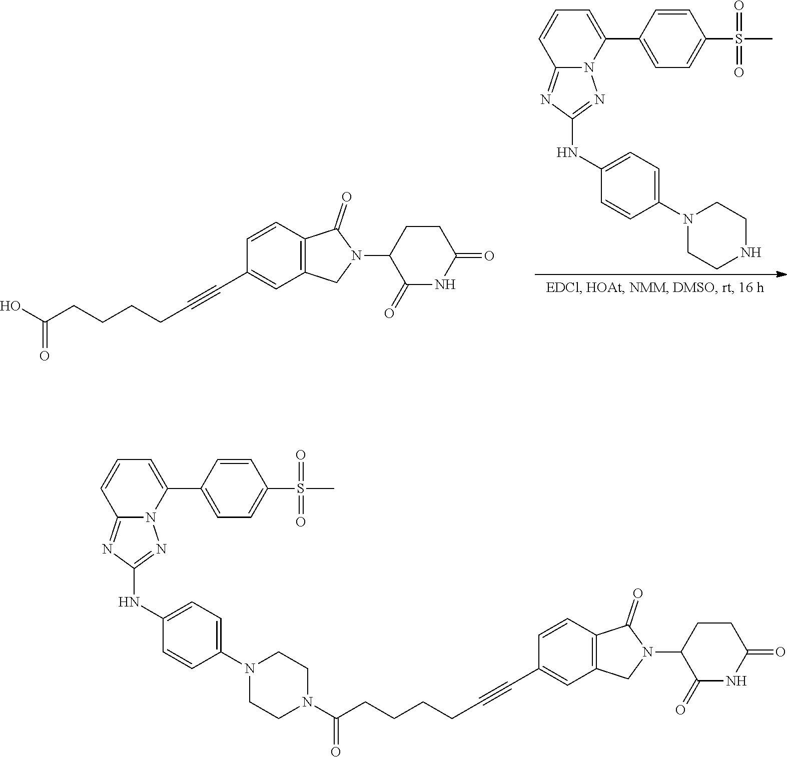

JA-250 was synthesized following the standard procedure for preparing JA-001 (8.6 mg, 35% yield) as a light yellow solid. MS (ESI) m/z: 799.3 [M+H]⁺.

### Example 339. 3-(5-(7-(4-(4-((5-(4-(Methylsulfonyl)phenyl)-[1,2,4]triazolo[1,5-a]pyridin-2-yl)amino)phenyl)piperazin-1-yl)-7-oxoheptyl)-1-oxoisoindolin-2-yl)piperidine-2,6-dione (JA-251)

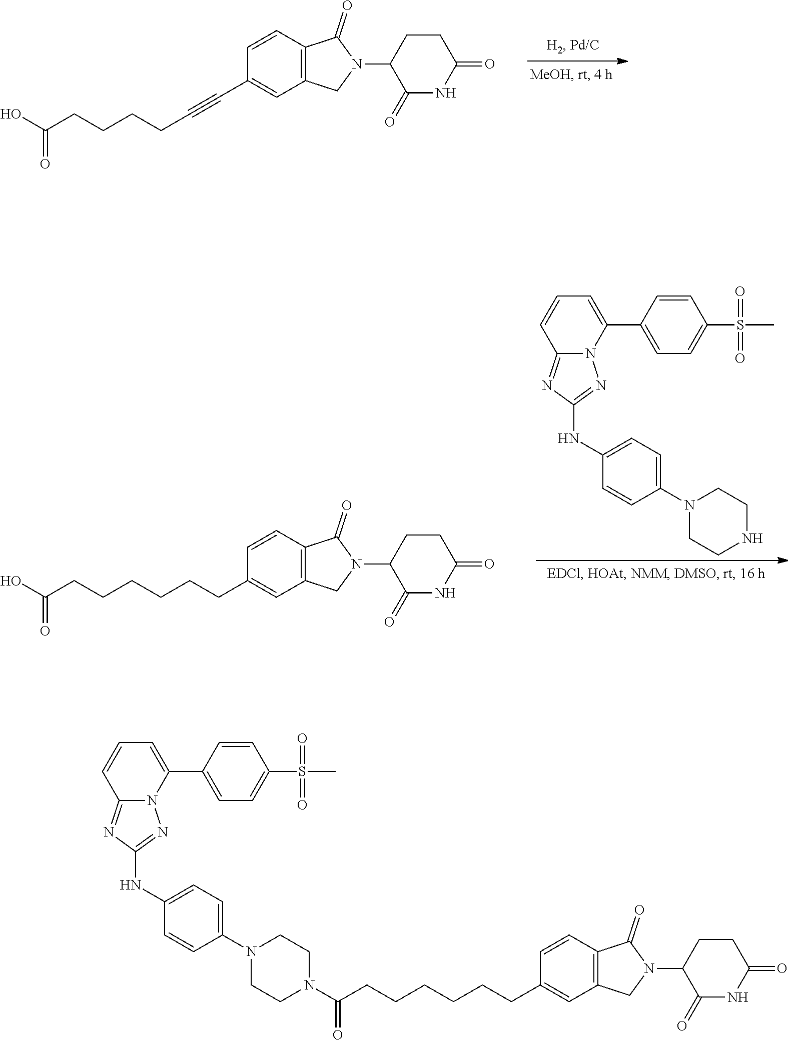

### Step 1: Synthesis of 7-(2-(2,6-dioxopiperidin-3-yl)-1-oxoisoindolin-5-yl)heptanoic acid

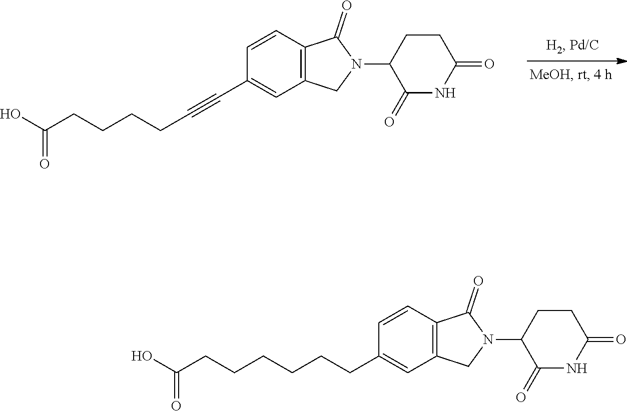 [SMILES: O=C(O)CCCCC#Cc1ccc2c(c1)CN(C1CCC(=O)NC1=O)C2=O ; O=C(O)CCCCCCc1ccc2c(c1)CN(C1CCC(=O)NC1=O)C2=O]

A mixture of 7-(2-(2,6-dioxopiperidin-3-yl)-1-oxoisoindolin-5-yl)hept-6-ynoic acid (100 mg, 261 umol) and Pd/C (5.5 mg, 2.6 umol) in MeOH (10 mL) was stirred at 25° C. for 16 h. The reaction mixture was purified by reverse-phase chromatography to give the desired product (67 mg, 68% yield) as a light yellow solid. MS (ESI) m/z: 371.2 [M−H]⁻.

### Step 2: Synthesis of 3-(5-(7-(4-(4-((5-(4-(methylsulfonyl)phenyl)-[1,2,4]triazolo[1,5-a]pyridin-2-yl)amino)phenyl)piperazin-1-yl)-7-oxoheptyl)-1-oxoisoindolin-2-yl)piperidine-2,6-dione

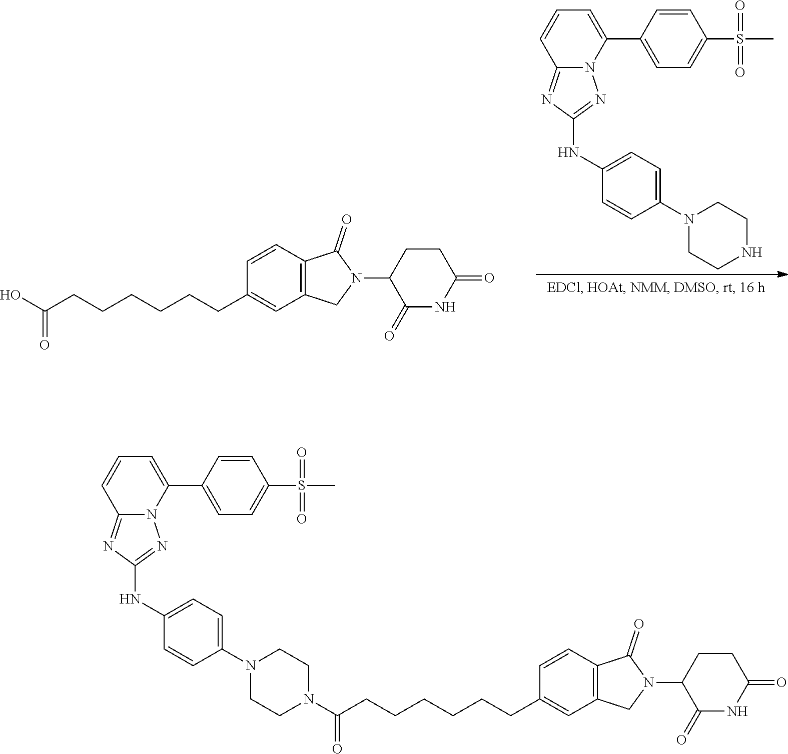

JA-251 was synthesized following the standard procedure for preparing JA-001 (7.3 mg, 34% yield) as a light yellow solid. MS (ESI) m/z: 803.3 [M+H]⁺.

### Example 340. 2-(2,6-Dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)hept-1-yn-1-yl)isoindoline-1,3-dione (JA-252)

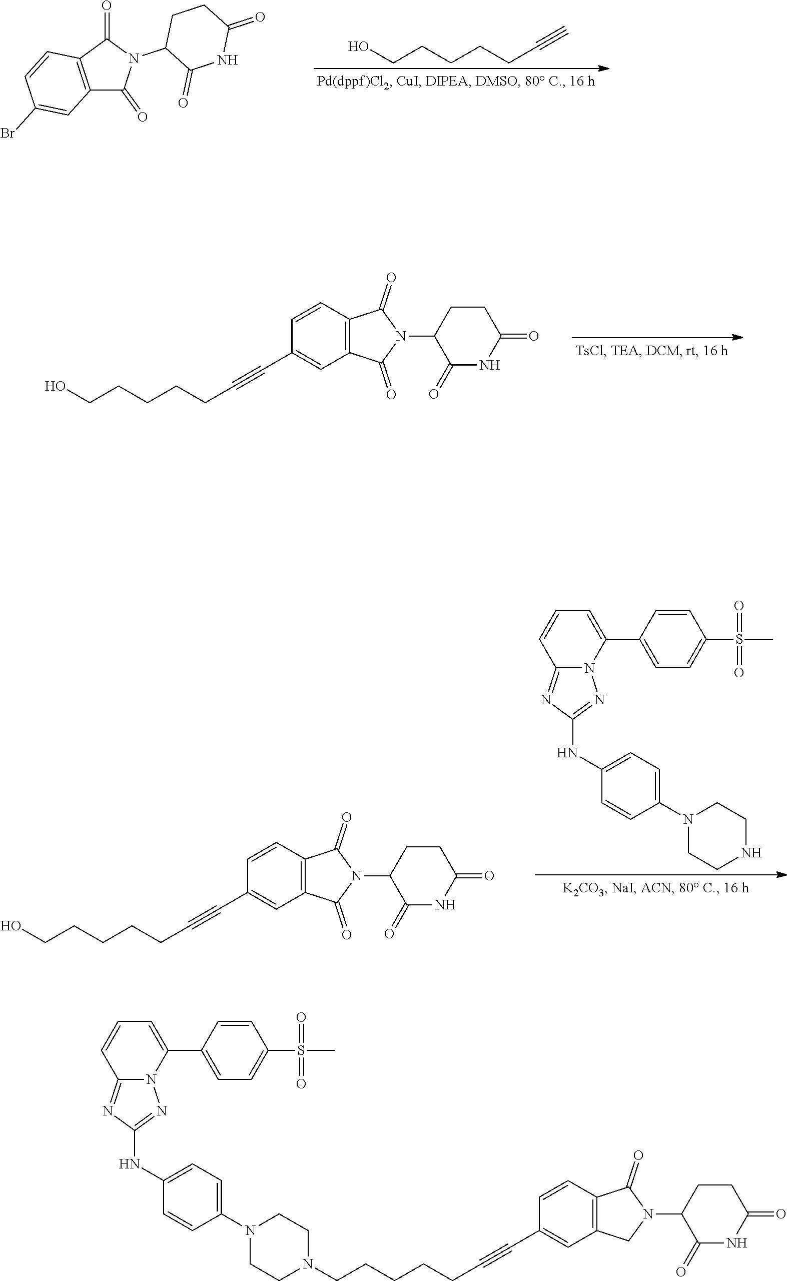

### Step 1: Synthesis of 2-(2,6-dioxopiperidin-3-yl)-5-(7-hydroxyhept-1-yn-1-yl)isoindoline-1,3-dione

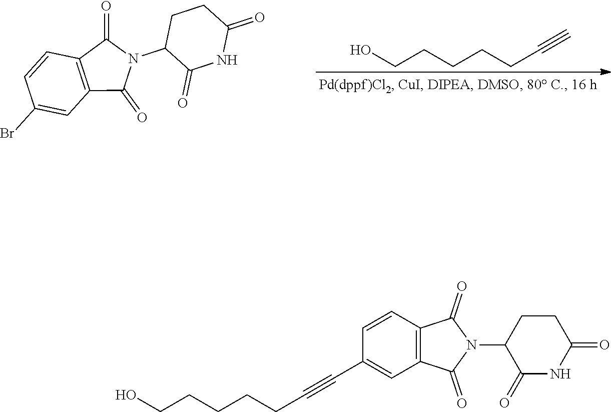

A mixture of 5-bromo-2-(2,6-dioxopiperidin-3-yl)isoindoline-1,3-dione (100 mg, 298 umol), Pd(dppf)Cl₂ (22 mg, 29.8 umol), hept-6-yn-1-ol (56 mg, 447 umol), CuI (6 mg, 29.8 umol) and DIPEA (116 mg, 894 umol) in DMSO (6 mL) was stirred at 80° C. for 16 h. The reaction mixture was purified by reverse-phase chromatography to give the desired product (61 mg, 48% yield) as a light yellow solid. MS (ESI) m/z: 369.1 [M+H]⁺.

### Step 2: Synthesis of 7-(2-(2,6-dioxopiperidin-3-yl)-1,3-dioxoisoindolin-5-yl)hept-6-yn-1-yl 4-methylbenzenesulfonate

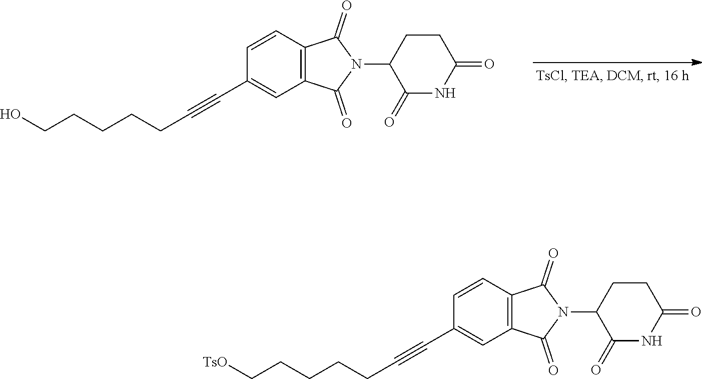 [SMILES: O=C1CCC(N2C(=O)c3ccc(C#CCCCCCO)cc3C2=O)C(=O)N1 ; Cc1ccc(S(=O)(=O)OCCCCCC#Cc2ccc3c(c2)C(=O)N(C2CCC(=O)NC2=O)C3=O)cc1]

To a solution of 2-(2,6-dioxopiperidin-3-yl)-5-(7-hydroxyhept-1-yn-1-yl)isoindoline-1,3-dione (50 mg, 136 umol) and TEA (27 mg, 272 umol) in DCM (5 mL) was added TsCl (39 mg, 204 umol) at room temperature. After the reaction was stirred at room temperature for 16 h, the mixture was concentrated and purified by reverse-phase chromatography to give the desired product (21 mg, 30% yield) as a light yellow solid. MS (ESI) m/z: 523.1 [M+H]⁺.

### Step 3: Synthesis of 2-(2,6-dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)hept-1-yn-1-yl)isoindoline-1,3-dione

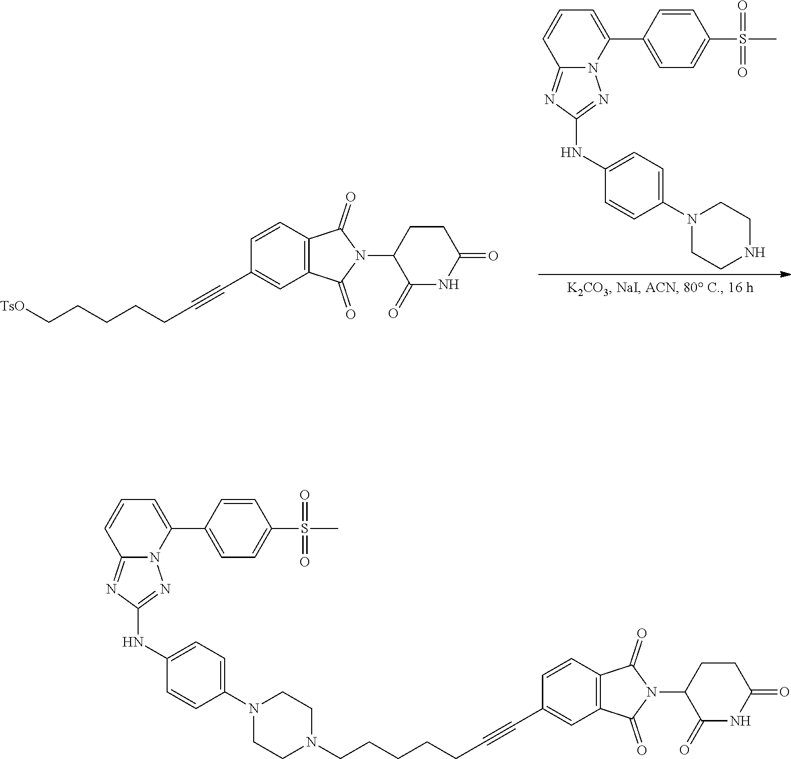

A mixture of 7-(2-(2,6-dioxopiperidin-3-yl)-1,3-dioxoisoindolin-5-yl)hept-6-yn-1-yl 4-methylbenzenesulfonate (10 mg, 12.5 umol), K₂CO₃ (27 mg, 272 umol), 5-(4-(methylsulfonyl)phenyl)-N-(4-(piperazin-1-yl)phenyl)-[1,2,4]triazolo[1,5-a]pyridin-2-amine (39 mg, 204 umol) and NaI (27 mg, 272 umol) in CH₃CN (4 mL) was stirred at 80° C. for 16 h. The mixture was concentrated and purified by reverse-phase chromatography to give the desired product (6 mg, 30% yield) as a light yellow solid. MS (ESI) m/z: 799.3 [M+H]⁺.

### Example 341. 2-(2,6-Dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)heptyl)isoindoline-1,3-dione (JA-253)

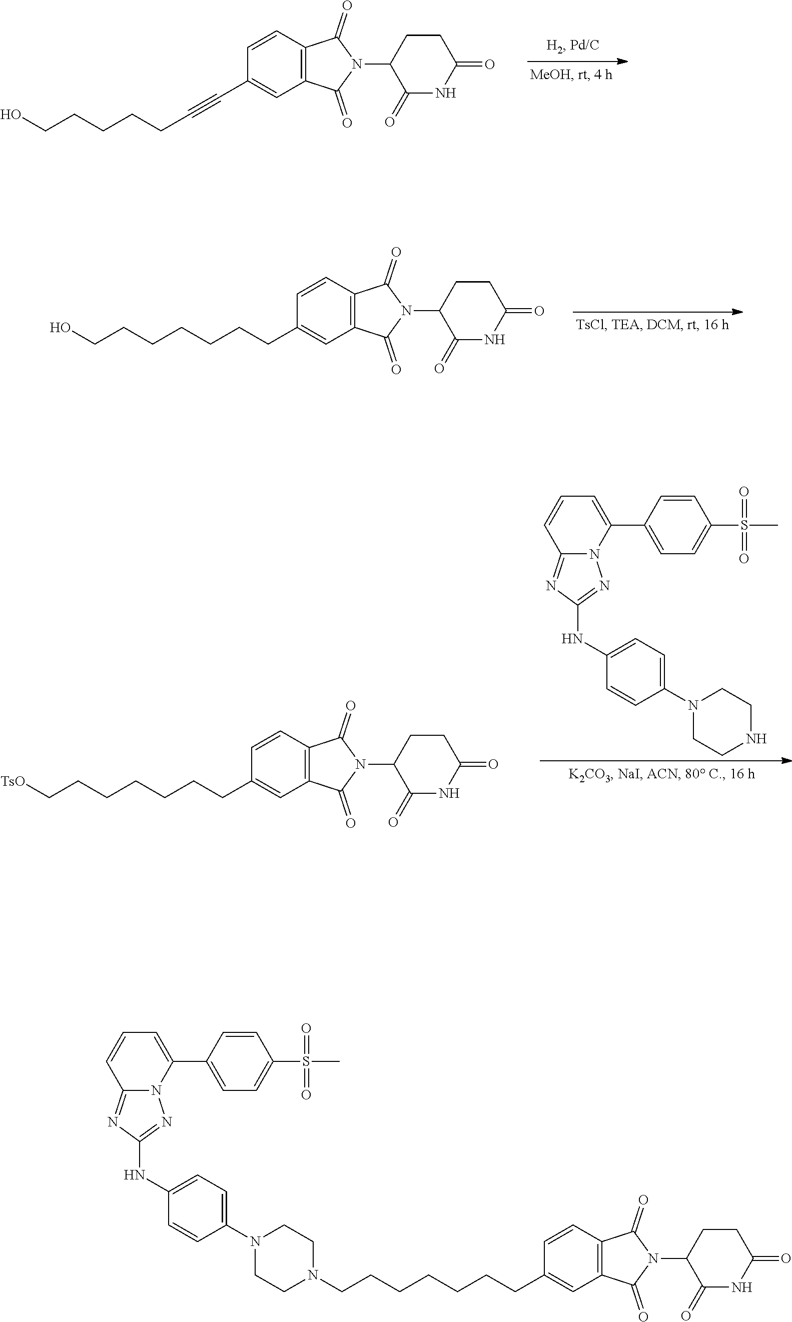

### Step 1: Synthesis of 2-(2,6-dioxopiperidin-3-yl)-5-(7-hydroxyheptyl)isoindoline-1,3-dione

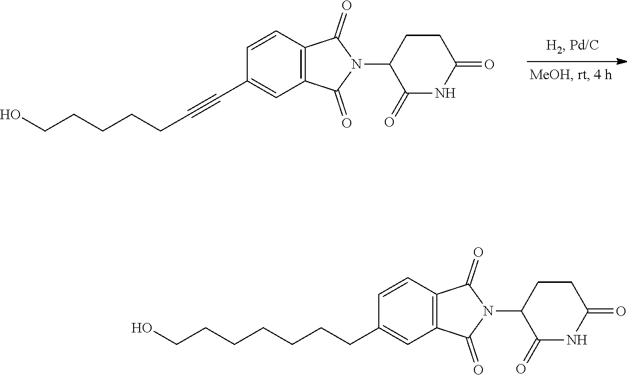 [SMILES: O=C1CCC(N2C(=O)c3ccc(C#CCCCCCO)cc3C2=O)C(=O)N1 ; O=C1CCC(N2C(=O)c3ccc(CCCCCCCO)cc3C2=O)C(=O)N1]

A mixture of 2-(2,6-dioxopiperidin-3-yl)-5-(7-hydroxyhept-1-yn-1-yl)isoindoline-1,3-dione (100 mg, 261 umol) and Pd/C (5.5 mg, 2.6 umol) in MeOH (10 mL) was stirred at 25° C. for 16 h, under hydrogen atmosphere. The reaction mixture was purified by reverse-phase chromatography to give the desired product (76 mg, 80% yield) as a light yellow solid. MS (ESI) m/z: 373.2 [M+H]⁺.

### Step 2: Synthesis of 7-(2-(2,6-dioxopiperidin-3-yl)-1,3-dioxoisoindolin-5-yl)heptyl 4-methylbenzenesulfonate

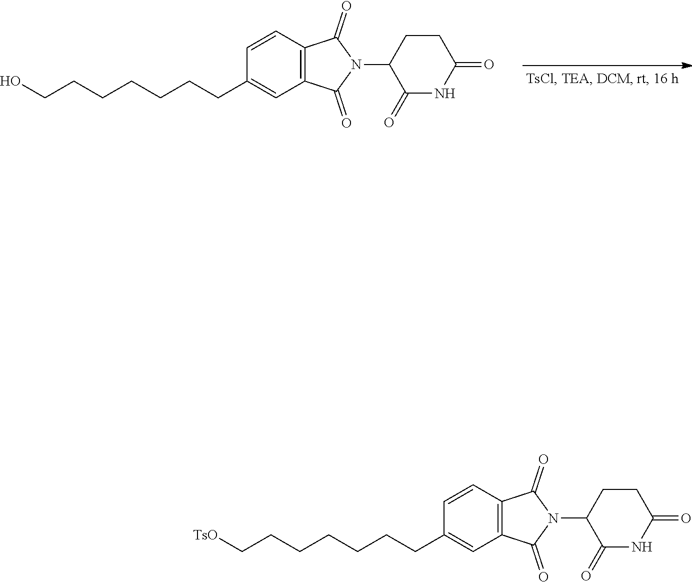 [SMILES: O=C1CCC(N2C(=O)c3ccc(CCCCCCCO)cc3C2=O)C(=O)N1 ; Cc1ccc(S(=O)(=O)OCCCCCCCc2ccc3c(c2)C(=O)N(C2CCC(=O)NC2=O)C3=O)cc1]

To a solution of 2-(2,6-dioxopiperidin-3-yl)-5-(7-hydroxyheptyl)isoindoline-1,3-dione (50 mg, 136 umol) and TEA (27 mg, 272 umol) in DCM (5 mL) was added TsCl (39 mg, 204 umol) at room temperature. After the reaction was stirred at room temperature for 16 h, the mixture was concentrated and purified by reverse-phase chromatography to give the desired product (24 mg, 32% yield) as a light yellow solid. MS (ESI) m/z: 527.2 [M+H]⁺.

### Step 3: Synthesis of 2-(2,6-dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)heptyl)isoindoline-1,3-dione

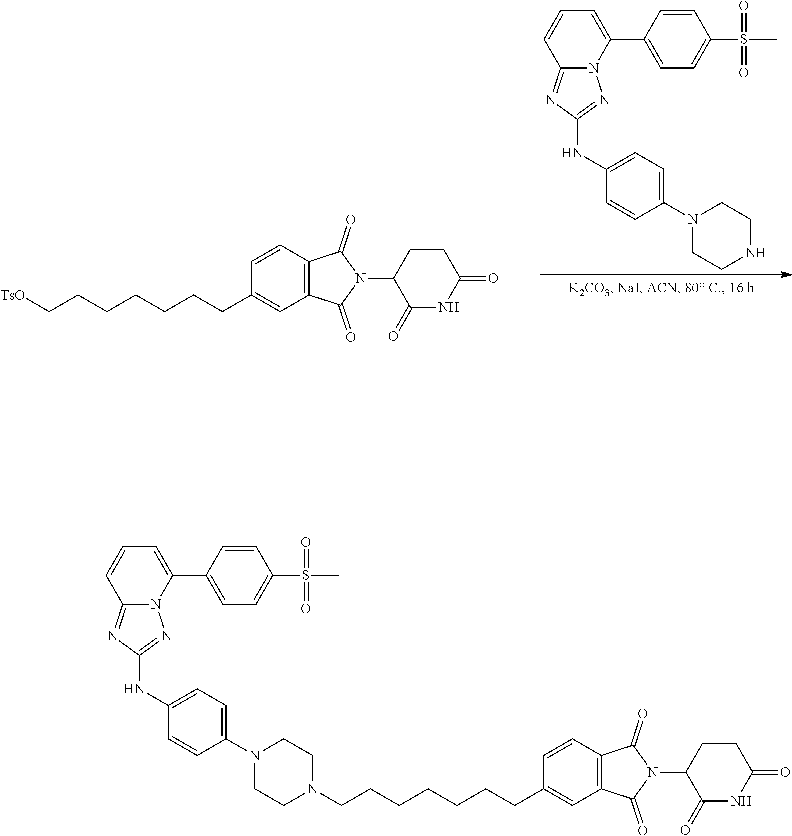

JA-253 was synthesized following the standard procedure for preparing JA-252 (7.5 mg, 36% yield) as a light yellow solid. MS (ESI) m/z: 803.3 [M+H]⁺.

### Example 342. 2-(2,6-Dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)hexyl)amino)isoindoline-1,3-dione (JA-254)

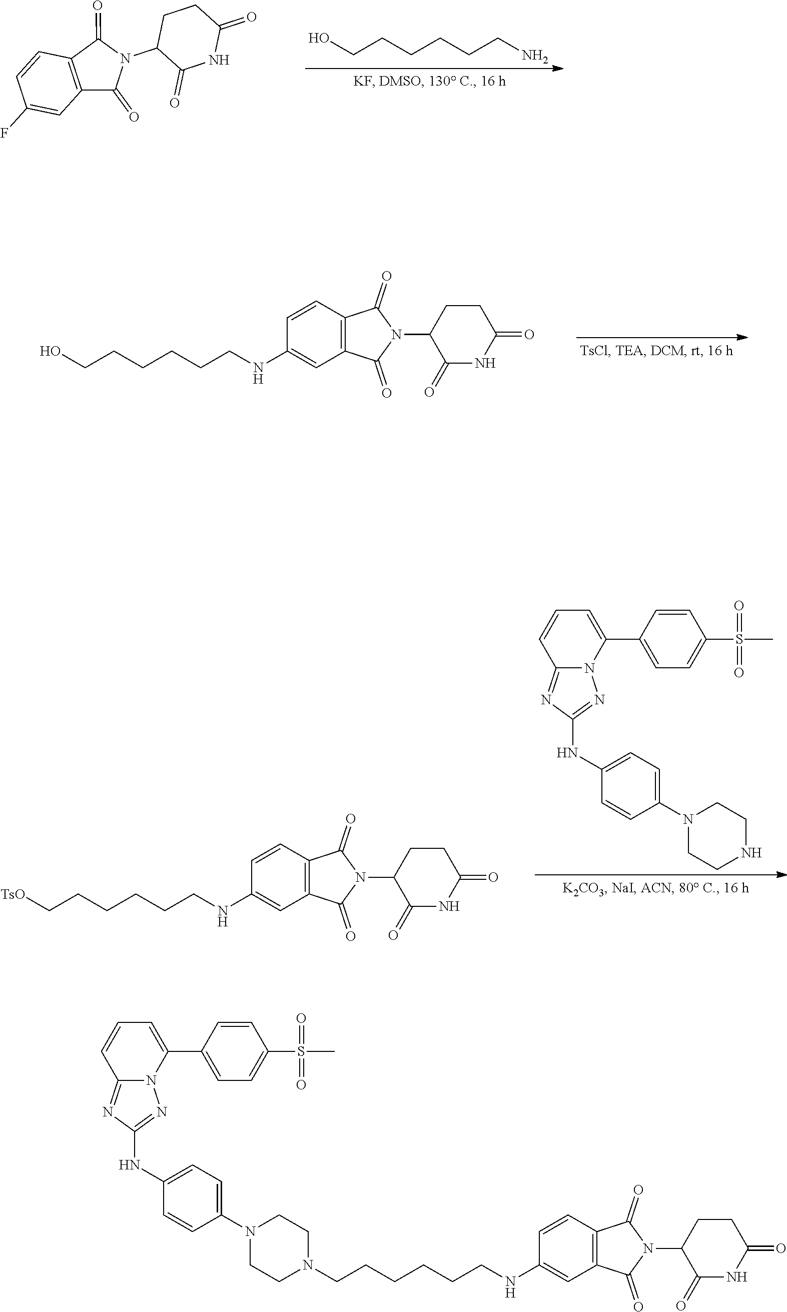

### Step 1: Synthesis of 2-(2,6-dioxopiperidin-3-yl)-5-((6-hydroxyhexyl)amino)isoindoline-1,3-dione

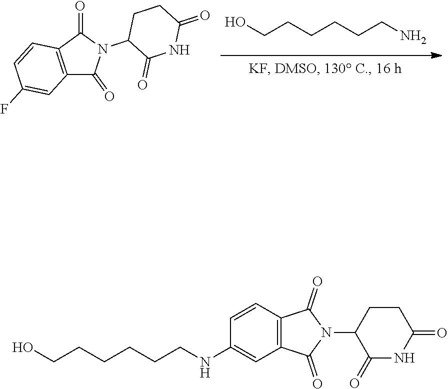

A mixture of 2-(2,6-dioxopiperidin-3-yl)-5-fluoroisoindoline-1,3-dione (100 mg, 0.36 mmol), KF (61 mg, 1.08 mmol) and 6-aminohexan-1-ol (64 mg, 0.54 mmol) in DMSO (10 mL) was stirred at 130° C. for 16 h. The reaction mixture was purified by reverse-phase chromatography to give the desired product (58 mg, 43% yield) as a little yellow solid. MS (ESI) m/z: 374.2 [M+H]⁺.

### Step 2: Synthesis of 6-((2-(2,6-dioxopiperidin-3-yl)-1,3-dioxoisoindolin-5-yl)amino)hexyl 4-methylbenzenesulfonate

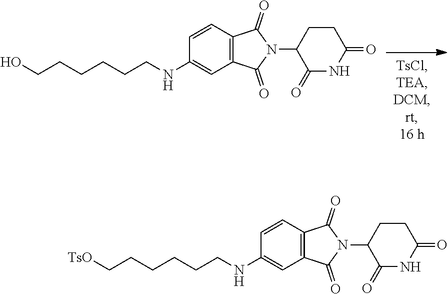 [SMILES: O=C1CCC(N2C(=O)c3ccc(NCCCCCCO)cc3C2=O)C(=O)N1 ; Cc1ccc(S(=O)(=O)OCCCCCCNc2ccc3c(c2)C(=O)N(C2CCC(=O)NC2=O)C3=O)cc1]

To a solution of 2-(2,6-dioxopiperidin-3-yl)-5-((6-hydroxyhexyl)amino)isoindoline-1,3-dione (50 mg, 136 umol) and TEA (27 mg, 272 umol) in DCM (5 mL) was added TsCl (39 mg, 204 umol) at room temperature. After the reaction was stirred at room temperature for 16 h, the mixture was concentrated and purified by reverse-phase chromatography to give the desired product (31 mg, 46% yield) as a light yellow solid. MS (ESI) m/z: 528.2 [M+H]⁺.

### Step 3: Synthesis 2-(2,6-dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)hexyl)amino)isoindoline-1,3-dione

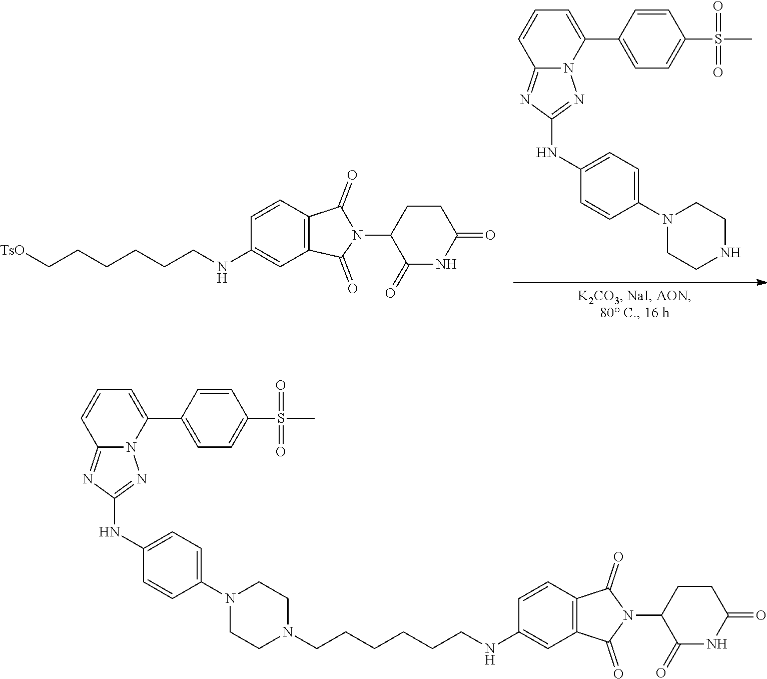

JA-254 was synthesized following the standard procedure for preparing JA-252 (8.9 mg, 39% yield) as a light yellow solid. MS (ESI) m/z: 804.3 [M+H]⁺.

### Example 343. 2-(2,6-Dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)-6-oxohexyl)oxy)isoindoline-1,3-dione (JA-255)

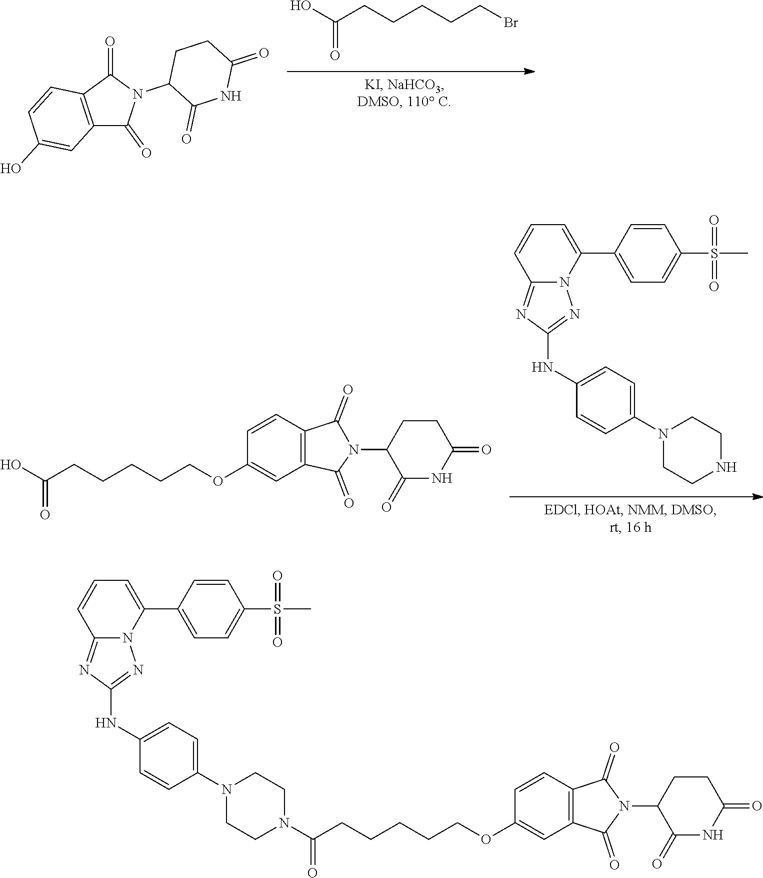

### Step 1: Synthesis of 6-((2-(2,6-dioxopiperidin-3-yl)-1,3-dioxoisoindolin-5-yl)oxy)hexanoic acid

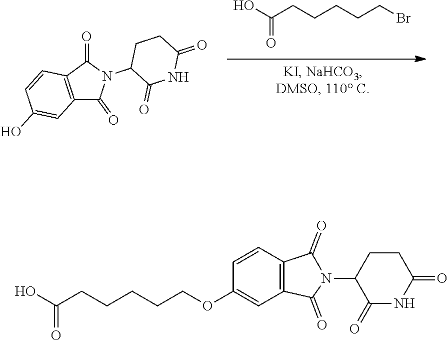

A mixture of 2-(2,6-dioxopiperidin-3-yl)-5-hydroxyisoindoline-1,3-dione (100 mg, 385 umol), NaHCO₃ (81 mg, 769 umol), 6-bromohexanoic acid (74 mg, 769 umol) and KI (64 mg, 385 umol) in DMSO (10 mL) was stirred at 110° C. for 16 h. The reaction mixture was purified by reverse-phase chromatography to give the desired product (65 mg, 44% yield) as a light yellow solid. MS (ESI) m/z: 387.1 [M−H]⁻.

### Step 2: Synthesis of 2-(2,6-dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)-6-oxohexyl)oxy)isoindoline-1,3-dione

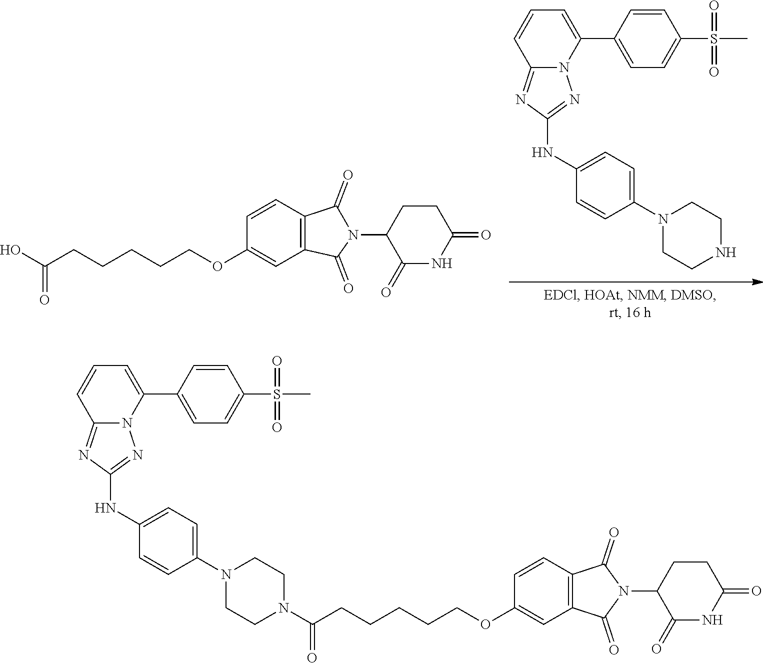

JA-255 was synthesized following the standard procedure for preparing JA-252 (7.5 mg, 35% yield) as a light yellow solid. MS (ESI) m/z: 819.3 [M+H]⁺.

### Example 344. 2-(2,6-Dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)hexyl)oxy)isoindoline-1,3-dione (JA-256)

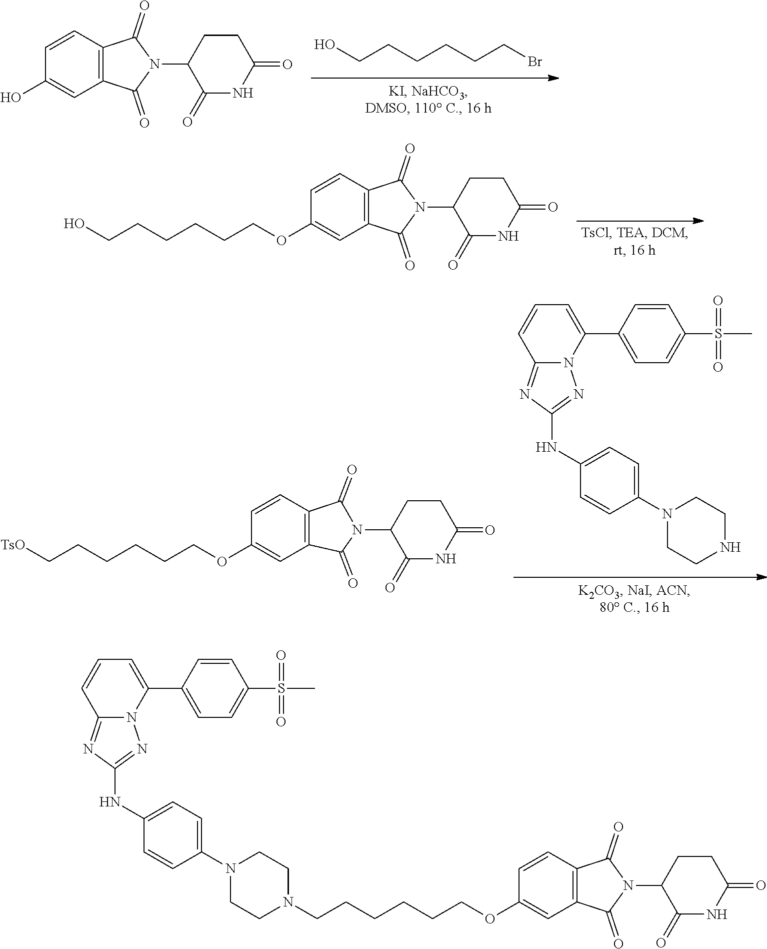

### Step 1: Synthesis of 2-(2,6-dioxopiperidin-3-yl)-5-((6-hydroxyhexyl)oxy)isoindoline-1,3-dione

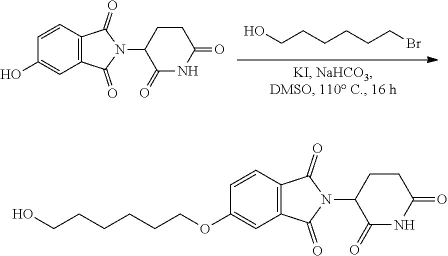 [SMILES: O=C1CCC(N2C(=O)c3ccc(O)cc3C2=O)C(=O)N1 ; OCCCCCCBr ; O=C1CCC(N2C(=O)c3ccc(OCCCCCCO)cc3C2=O)C(=O)N1]

A mixture of 2-(2,6-dioxopiperidin-3-yl)-5-hydroxyisoindoline-1,3-dione (100 mg, 385 umol), NaHCO₃ (81 mg, 769 umol), 6-bromohexan-1-ol (74 mg, 769 umol) and KI (64 mg, 385 umol) in DMSO (10 mL) was stirred at 110° C. for 16 h. The reaction mixture was purified by reverse-phase chromatography to give the desired product (52 mg, 40% yield) as a light yellow solid. MS (ESI) m/z: 375.1 [M+H]⁺.

### Step 2: Synthesis of 6-((2-(2,6-dioxopiperidin-3-yl)-1,3-dioxoisoindolin-5-yl)oxy)hexyl 4-methylbenzenesulfonate

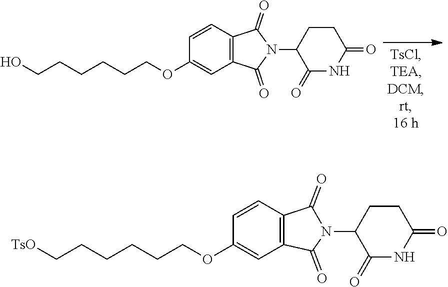

To a solution of 2-(2,6-dioxopiperidin-3-yl)-5-((6-hydroxyhexyl)oxy)isoindoline-1,3-dione (50 mg, 136 umol) and TEA (27 mg, 272 umol) in DCM (5 mL) was added TsCl (39 mg, 204 umol) at room temperature. After the reaction was stirred at room temperature for 16 h, the mixture was concentrated and purified by reverse-phase chromatography to give the desired product (31 mg, 46% yield) as a light yellow solid. MS (ESI) m/z: 529.2 [M+H]⁺.

### Step 3: Synthesis of 2-(2,6-dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)hexyl)oxy)isoindoline-1,3-dione

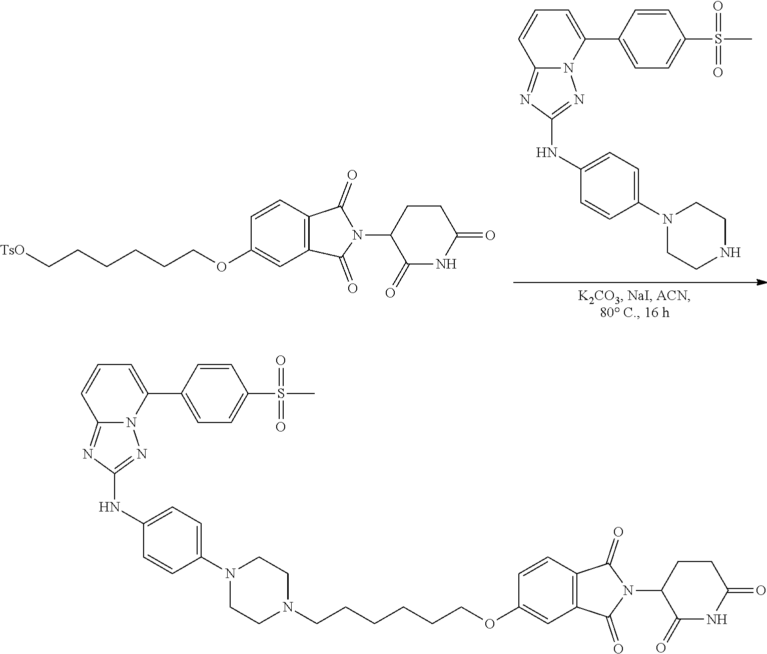

JA-256 was synthesized following the standard procedure for preparing JA-252 (10.5 mg, 43% yield) as a light yellow solid. MS (ESI) m/z: 805.3 [M+H]⁺.

### Example 345. 5-(7-(4-(4-(8-(3,5-Difluoro-4-(morpholinomethyl)phenyl)quinoxalin-2-yl)-1H-pyrazol-1-yl)piperidin-1-yl)-7-oxoheptyl)-2-(2,6-dioxopiperidin-3-yl)isoindoline-1,3-dione (JA-257)

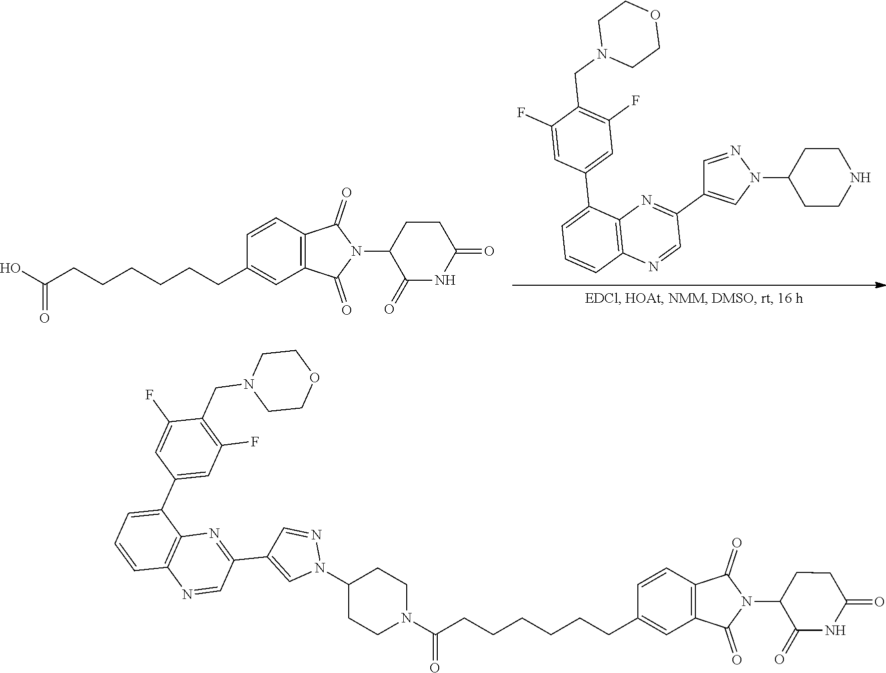

JA-257 was synthesized following the standard procedure for preparing JA-001 (7.4 mg, 34% yield) as a light yellow solid. MS (ESI) m/z: 859.4 [M+H]⁺.

### Example 346. 5-(7-(4-(4-(8-(3,5-Difluoro-4-(morpholinomethyl)phenyl)quinoxalin-2-yl)-1H-pyrazol-1-yl)piperidin-1-yl)-7-oxohept-1-yn-1-yl)-2-(2,6-dioxopiperidin-3-yl)isoindoline-1,3-dione (JA-258)

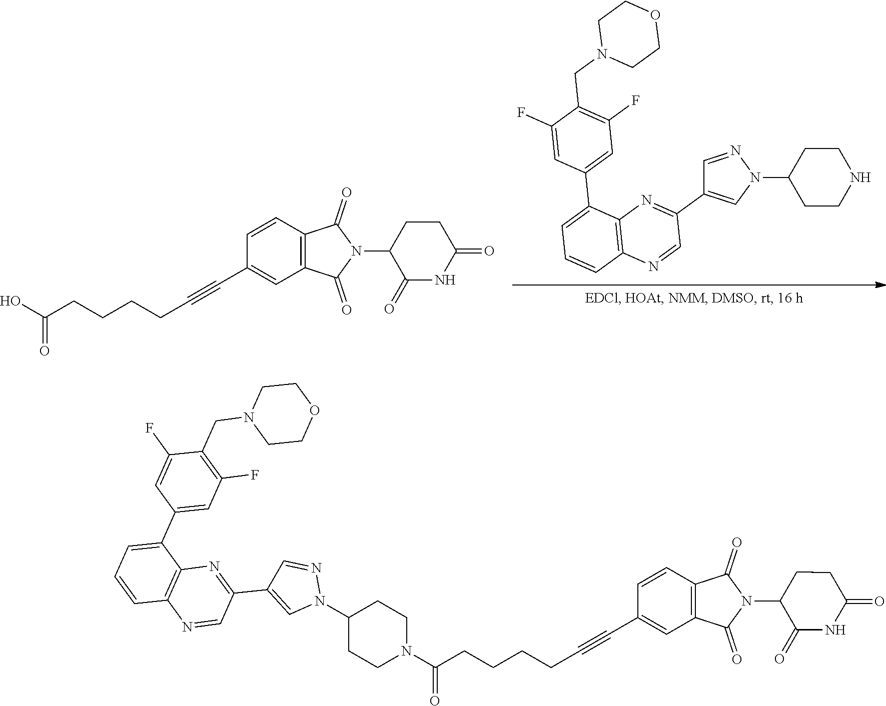

JA-258 was synthesized following the standard procedure for preparing JA-001 (7.9 mg, 37% yield) as a light yellow solid. MS (ESI) m/z: 855.3 [M+H]⁺.

### Example 347. 3-(6-(7-(4-(4-(8-(3,5-Difluoro-4-(morpholinomethyl)phenyl)quinoxalin-2-yl)-1H-pyrazol-1-yl)piperidin-1-yl)-7-oxohept-1-yn-1-yl)-1-oxoisoindolin-2-yl)piperidine-2,6-dione (JA-259)

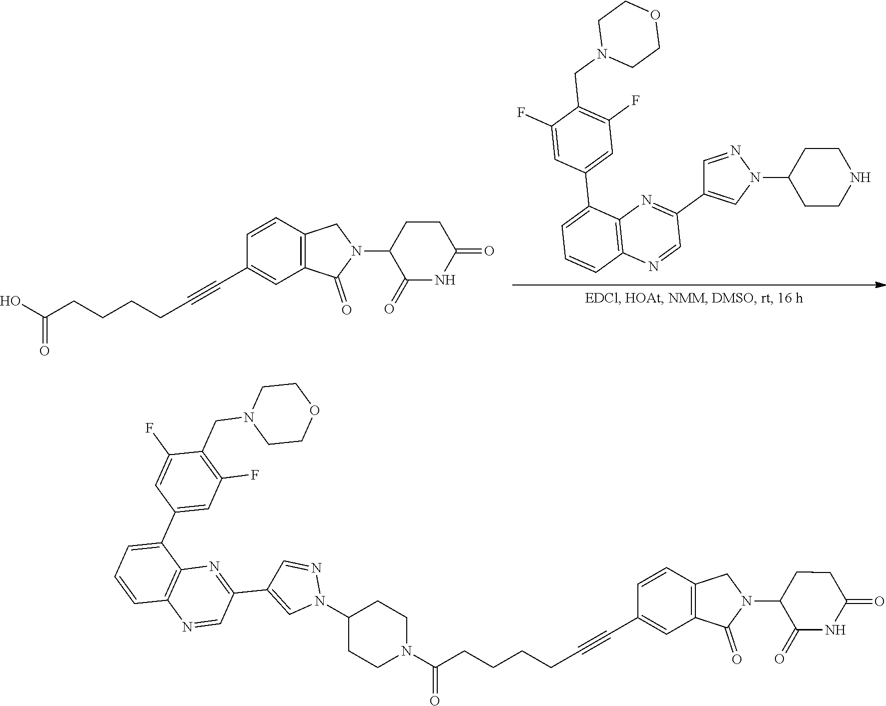

JA-259 was synthesized following the standard procedure for preparing JA-001 (8.3 mg, 37% yield) as a light yellow solid. MS (ESI) m/z: 841.4 [M+H]⁺.

### Example 348. 5-((6-(4-(4-(8-(3,5-Difluoro-4-(morpholinomethyl)phenyl)quinoxalin-2-yl)-1H-pyrazol-1-yl)piperidin-1-yl)-6-oxohexyl)oxy)-2-(2,6-dioxopiperidin-3-yl)isoindoline-1,3-dione (JA-260)

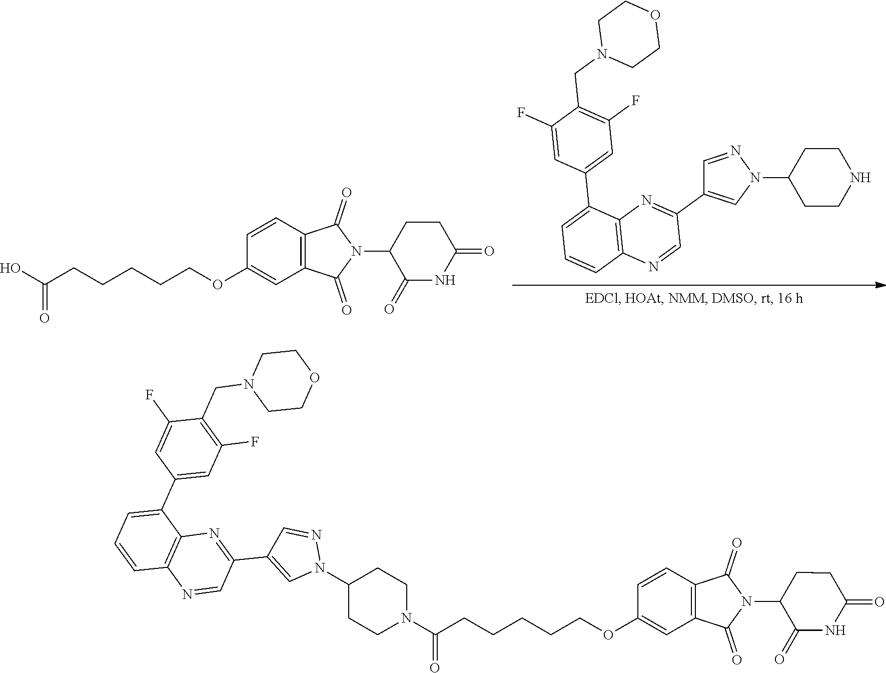

JA-260 was synthesized following the standard procedure for preparing JA-001 (9.2 mg, 41% yield) as a light yellow solid. MS (ESI) m/z: 861.3 [M+H]⁺.

### Example 349. 3-(5-((6-(4-(4-((5-(4-(Methylsulfonyl)phenyl)-[1,2,4]triazolo[1,5-a]pyridin-2-yl)amino)phenyl)piperazin-1-yl)-6-oxohexyl)oxy)-1-oxoisoindolin-2-yl)piperidine-2,6-dione (JA-261)

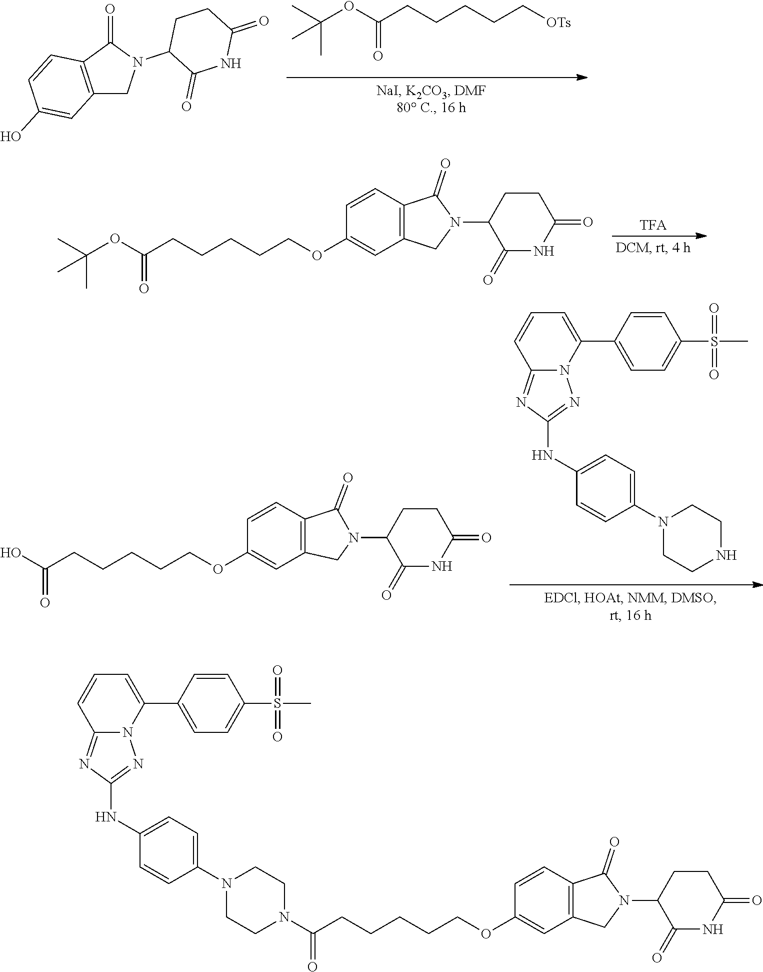

### Step 1: Synthesis of tert-butyl 6-((2-(2,6-dioxopiperidin-3-yl)-1-oxoisoindolin-5-yl)oxy)hexanoate

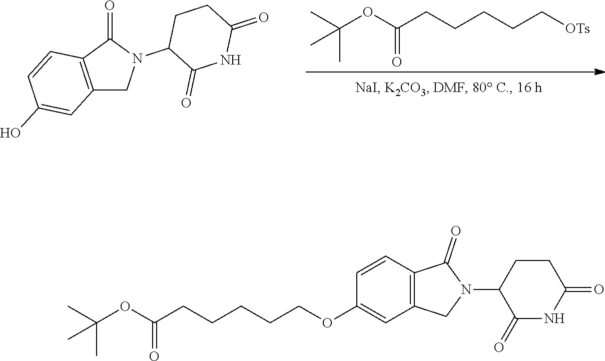 [SMILES: O=C1CCC(N2Cc3cc(O)ccc3C2=O)C(=O)N1 ; Cc1ccc(S(=O)(=O)OCCCCCC(=O)OC(C)(C)C)cc1 ; CC(C)(C)OC(=O)CCCCCOc1ccc2c(c1)CN(C1CCC(=O)NC1=O)C2=O]

A mixture of 3-(5-hydroxy-1-oxoisoindolin-2-yl)piperidine-2,6-dione (100 mg, 385 umol), K₂CO₃ (106 mg, 769 umol), NaI (22 mg, 385 umol) and tert-butyl 6-(tosyloxy) hexanoate (263 mg, 769 umol) in DMF (10 mL) was stirred at 80° C. for 16 h. The mixture was concentrated and purified by reverse-phase chromatography to give the desired product (32 mg, 22% yield) as a light yellow solid. MS (ESI) m/z: 431.2 [M+H]⁺.

### Step 2: Synthesis of 6-((2-(2,6-dioxopiperidin-3-yl)-1-oxoisoindolin-5-yl)oxy)hexanoic acid

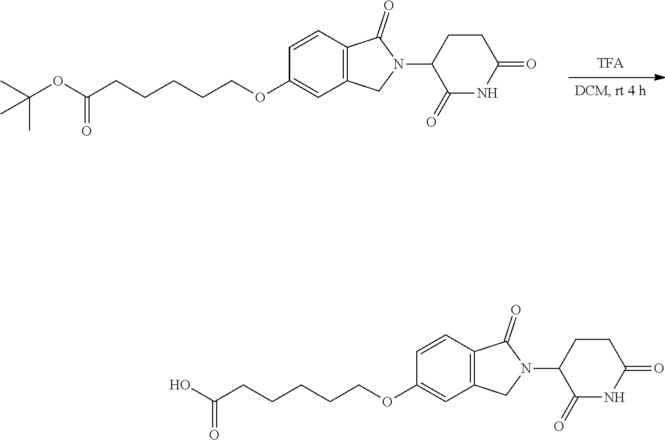 [SMILES: CC(C)(C)OC(=O)CCCCCOc1ccc2c(c1)CN(C1CCC(=O)NC1=O)C2=O ; O=C(O)CCCCCOc1ccc2c(c1)CN(C1CCC(=O)NC1=O)C2=O]

To a solution of tert-butyl 6-((2-(2,6-dioxopiperidin-3-yl)-1-oxoisoindolin-5-yl)oxy)hexanoate (30 mg, 69.7 umol) in DCM (5 mL) was added TFA (4 mL). After the reaction was stirred at room temperature for 2 h, the mixture was concentrated and purified by reverse-phase chromatography to give the desired product (24 mg, 92% yield) as a light yellow solid. MS (ESI) m/z: 372.1 [M−H]⁻.

### Step 3: Synthesis of 3-(5-((6-(4-(4-((5-(4-(methylsulfonyl)phenyl)-[1,2,4]triazolo[1,5-a]pyridin-2-yl)amino)phenyl)piperazin-1-yl)-6-oxohexyl)oxy)-1-oxoisoindolin-2-yl)piperidine-2,6-dione

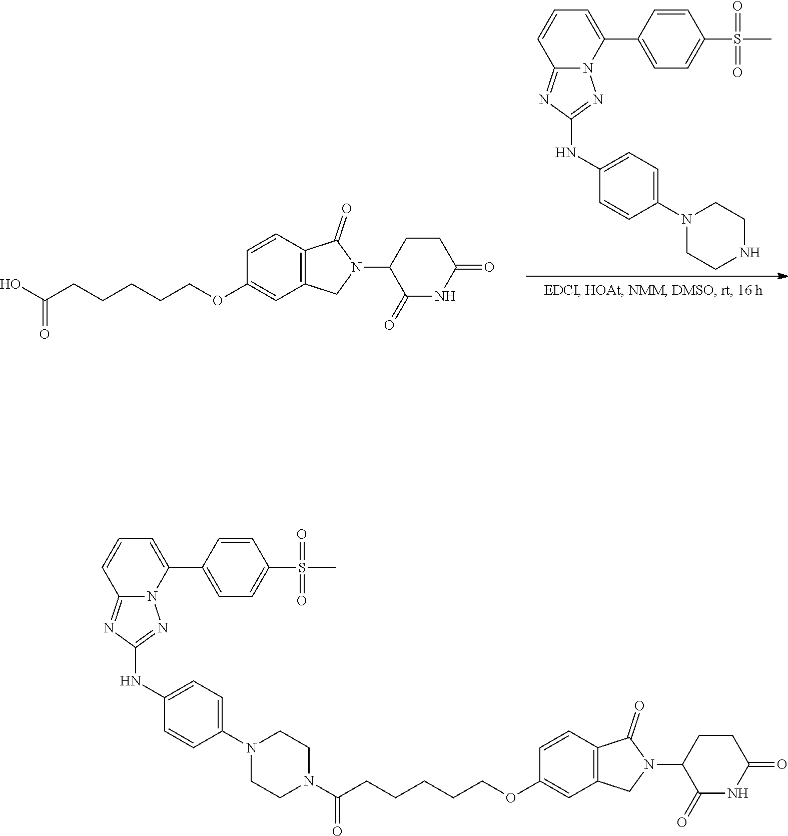 [SMILES: O=C(O)CCCCCOc1ccc2c(c1)CN(C1CCC(=O)NC1=O)C2=O ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCOc6ccc7c(c6)CN(C6CCC(=O)NC6=O)C7=O)CC5)cc4)nn23)cc1]

JA-261 was synthesized following the standard procedure for preparing JA-001 (11 mg, 42% yield) as a light yellow solid. MS (ESI) m/z: 805.3 [M+H]⁺.

### Example 350. 3-(6-(7-(4-(4-((5-(4-(Methylsulfonyl)phenyl)-[1,2,4]triazolo[1,5-a]pyridin-2-yl)amino)phenyl)piperazin-1-yl)-7-oxohept-1-yn-1-yl)-1-oxoisoindolin-2-yl)piperidine-2,6-dione (JA-262)

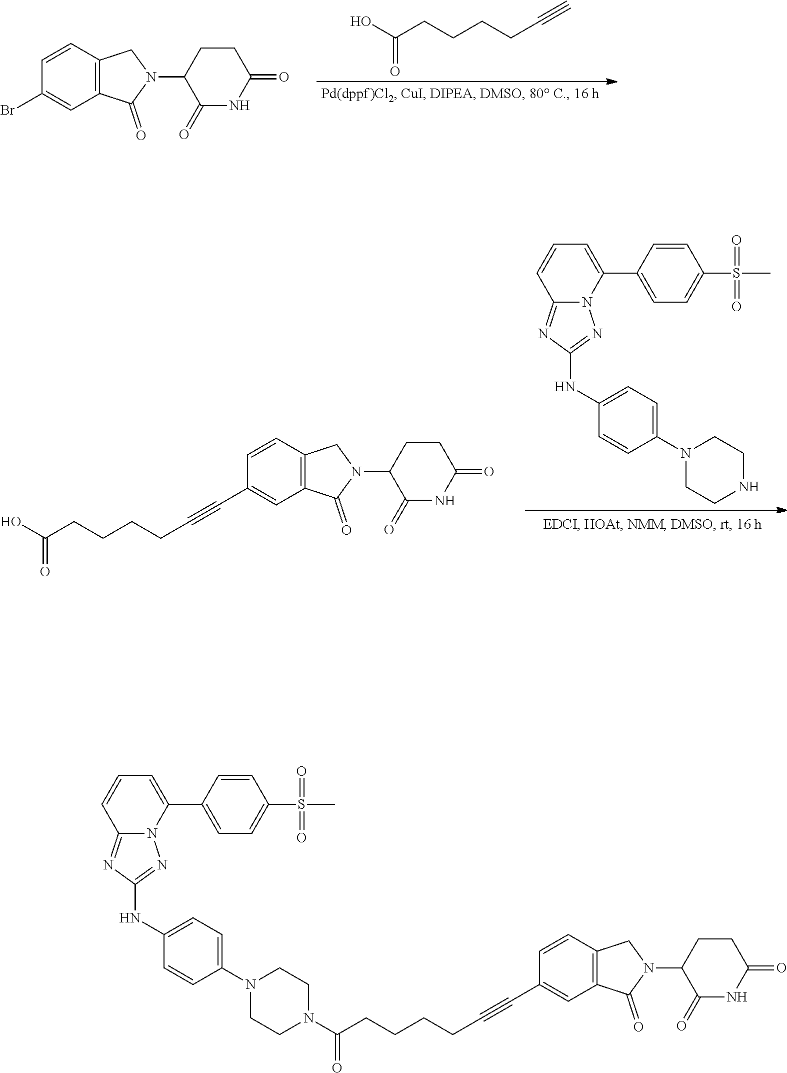

### Step 1: Synthesis of 7-(2-(2,6-dioxopiperidin-3-yl)-3-oxoisoindolin-5-yl)hept-6-ynoic acid

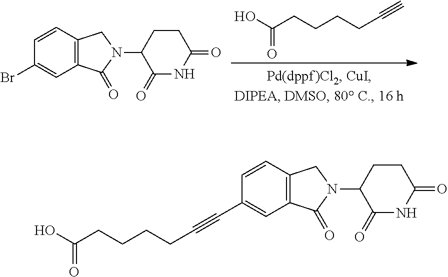

A mixture of 3-(6-bromo-1-oxoisoindolin-2-yl)piperidine-2,6-dione (100 mg, 298 umol), Pd(dppf)Cl₂ (22 mg, 29.8 umol), hept-6-ynoic acid (56 mg, 447 umol), DIPEA (116 mg, 894 umol) and CuI (6 mg, 29.8 umol) in DMSO (6 mL) was stirred at 80° C. for 16 h. The reaction mixture was purified by reverse-phase chromatography to give the desired product (49 mg, 45% yield) as a light yellow solid. MS (ESI) m/z: 367.1 [M−H]⁻.

### Step 2: Synthesis of 3-(6-(7-(4-(4-((5-(4-(methylsulfonyl)phenyl)-[1,2,4]triazolo[1,5-a]pyridin-2-yl)amino)phenyl)piperazin-1-yl)-7-oxohept-1-yn-1-yl)-1-oxoisoindolin-2-yl)piperidine-2,6-dione

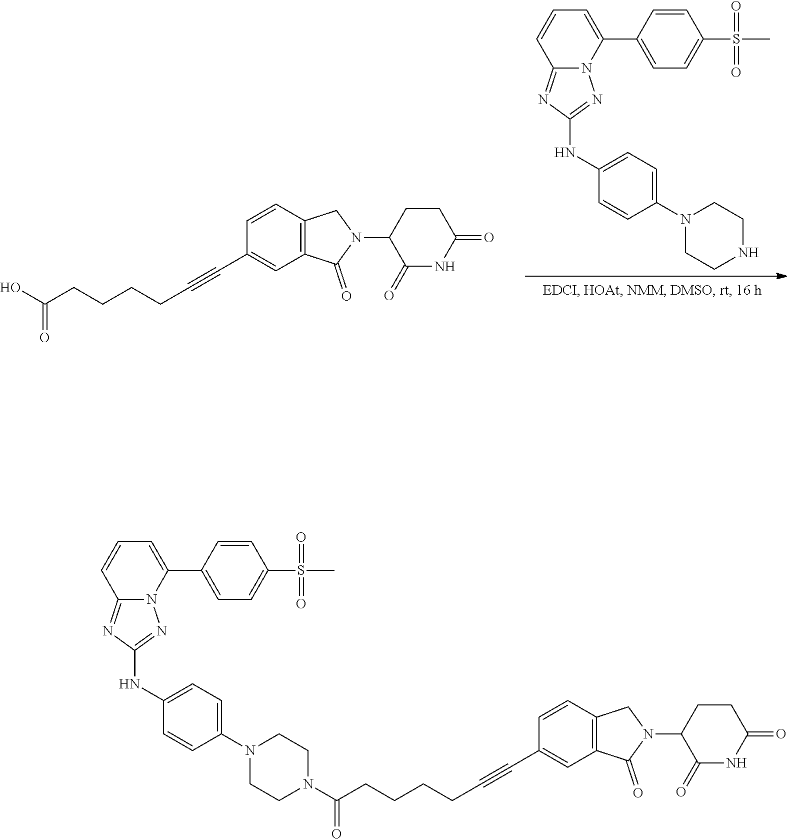 [SMILES: O=C(O)CCCCC#Cc1ccc2c(c1)C(=O)N(C1CCC(=O)NC1=O)C2 ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCC#Cc6ccc7c(c6)C(=O)N(C6CCC(=O)NC6=O)C7)CC5)cc4)nn23)cc1]

JA-262 was synthesized following the standard procedure for preparing JA-001 (10 mg, 40% yield) as a light yellow solid. MS (ESI) m/z: 799.3 [M+H]⁺.

### Example 351. 3-(5-((6-(4-(4-(8-(3,5-Difluoro-4-(morpholinomethyl)phenyl)quinoxalin-2-yl)-1H-pyrazol-1-yl)piperidin-1-yl)-6-oxohexyl)oxy)-1-oxoisoindolin-2-yl)piperidine-2,6-dione (JA-263)

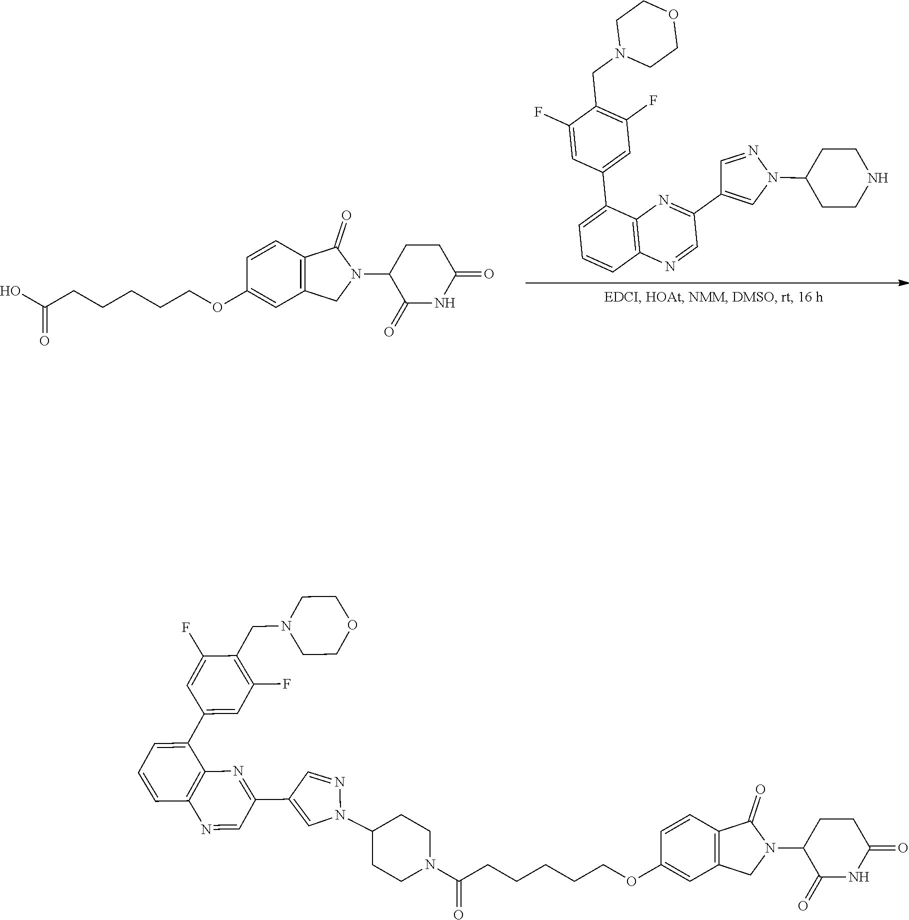 [SMILES: O=C(O)CCCCCOc1ccc2c(c1)CN(C1CCC(=O)NC1=O)C2=O ; Fc1cc(-c2cccc3ncc(-c4cnn(C5CCNCC5)c4)nc23)cc(F)c1CN1CCOCC1 ; O=C1CCC(N2Cc3cc(OCCCCCC(=O)N4CCC(n5cc(-c6cnc7cccc(-c8cc(F)c(CN9CCOCC9)c(F)c8)c7n6)cn5)CC4)ccc3C2=O)C(=O)N1]

JA-263 was synthesized following the standard procedure for preparing JA-001 (13 mg, 38% yield) as a light yellow solid. MS (ESI) m/z: 847.4 [M+H]⁺.

### Example 352. 5-(7-(4-(4-(8-(3,5-Difluoro-4-(morpholinomethyl)phenyl)quinoxalin-2-yl)-1H-pyrazol-1-yl)piperidin-1-yl)hept-1-yn-1-yl)-2-(2,6-dioxopiperidin-3-yl)isoindoline-1,3-dione (JA-264)

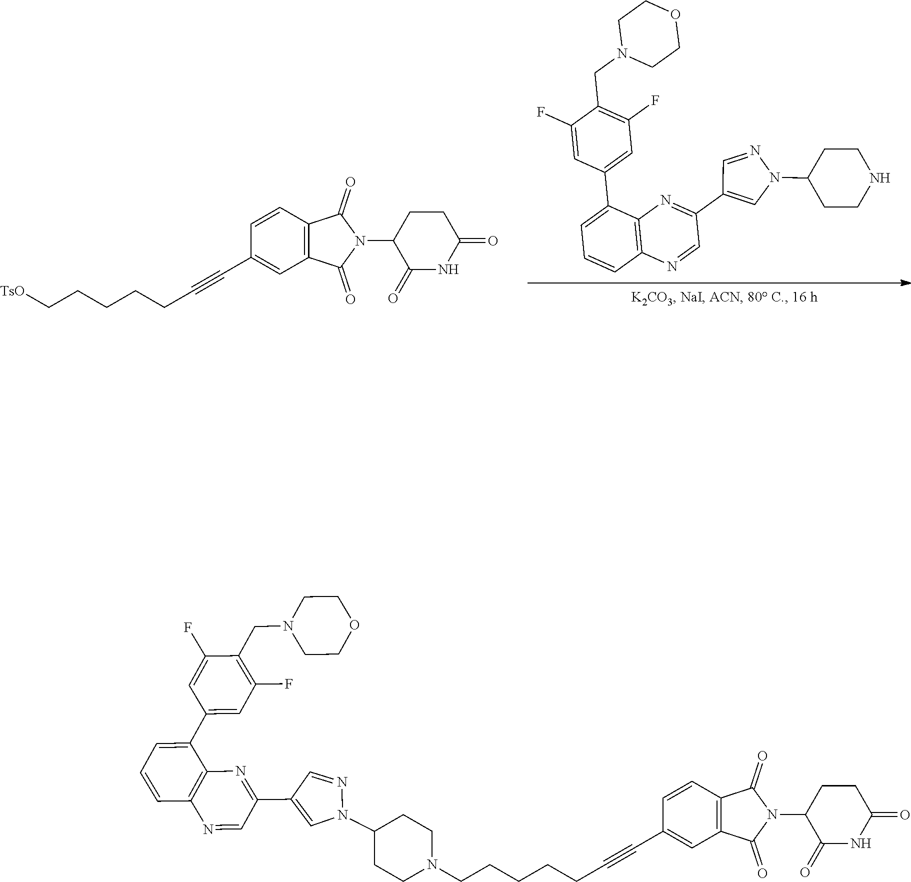 [SMILES: Cc1ccc(S(=O)(=O)OCCCCCC#Cc2ccc3c(c2)C(=O)N(C2CCC(=O)NC2=O)C3=O)cc1 ; Fc1cc(-c2cccc3ncc(-c4cnn(C5CCNCC5)c4)nc23)cc(F)c1CN1CCOCC1 ; O=C1CCC(N2C(=O)c3ccc(C#CCCCCCN4CCC(n5cc(-c6cnc7cccc(-c8cc(F)c(CN9CCOCC9)c(F)c8)c7n6)cn5)CC4)cc3C2=O)C(=O)N1]

A mixture of 7-(2-(2,6-dioxopiperidin-3-yl)-1,3-dioxoisoindolin-5-yl)hept-6-yn-1-yl 4-methylbenzenesulfonate (15 mg, 28 umol), K₂CO₃ (12 mg, 84 umol), NaI (4 mg, 28 umol) and 4-(2,6-difluoro-4-(3-(1-(piperidin-4-yl)-1H-pyrazol-4-yl)quinoxalin-5-yl)benzyl)morpholine (14 mg, 28 umol) in DMF (10 mL) was stirred at 80° C. for 16 h. The mixture was concentrated and purified by reverse-phase chromatography to give the desired product (7.6 mg, 32% yield) as a light yellow solid. MS (ESI) m/z: 841.4 [M+H]⁺.

### Example 353. 5-(7-(4-(4-(8-(3,5-Difluoro-4-(morpholinomethyl)phenyl)quinoxalin-2-yl)-1H-pyrazol-1-yl)piperidin-1-yl)heptyl)-2-(2,6-dioxopiperidin-3-yl)isoindoline-1,3-dione (JA-265)

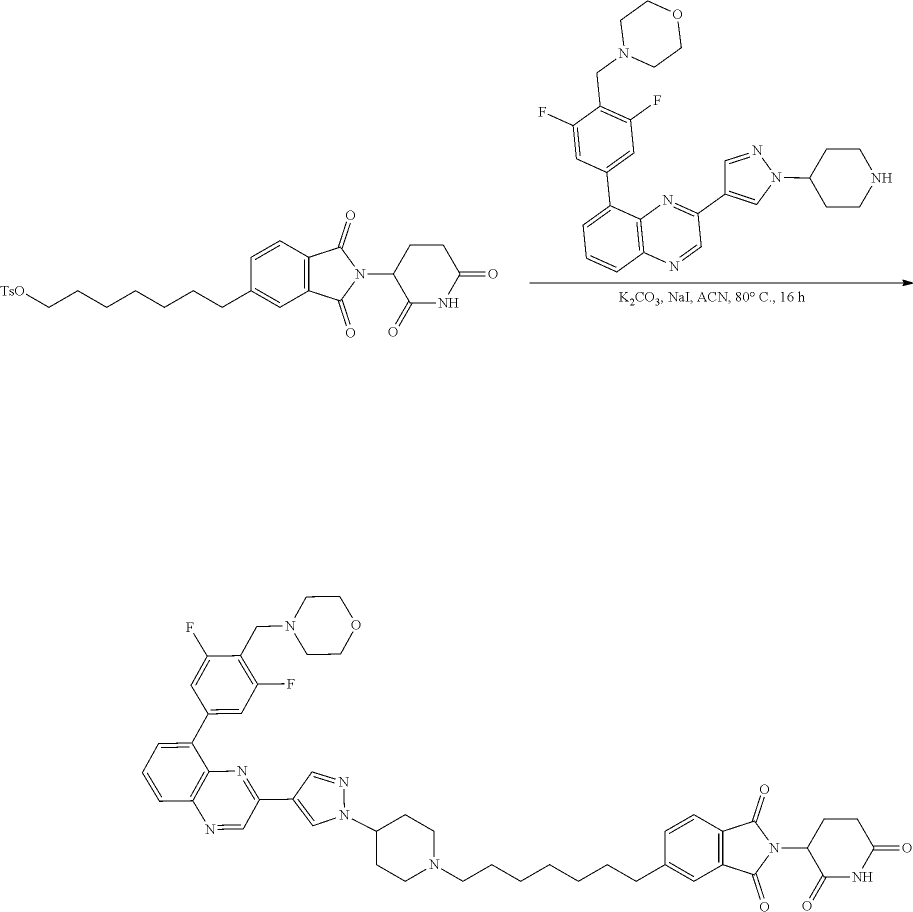 [SMILES: Cc1ccc(S(=O)(=O)OCCCCCCCc2ccc3c(c2)C(=O)N(C2CCC(=O)NC2=O)C3=O)cc1 ; Fc1cc(-c2cccc3ncc(-c4cnn(C5CCNCC5)c4)nc23)cc(F)c1CN1CCOCC1 ; O=C1CCC(N2C(=O)c3ccc(CCCCCCCN4CCC(n5cc(-c6cnc7cccc(-c8cc(F)c(CN9CCOCC9)c(F)c8)c7n6)cn5)CC4)cc3C2=O)C(=O)N1]

JA-265 was synthesized following the standard procedure for preparing JA-264 (8.3 mg, 38% yield) as a light yellow solid. MS (ESI) m/z: 845.4 [M+H]⁺.

### Example 354. 5-((6-(4-(4-(8-(3,5-Difluoro-4-(morpholinomethyl)phenyl)quinoxalin-2-yl)-1H-pyrazol-1-yl)piperidin-1-yl)hexyl)amino)-2-(2,6-dioxopiperidin-3-yl)isoindoline-1,3-dione (JA-266)

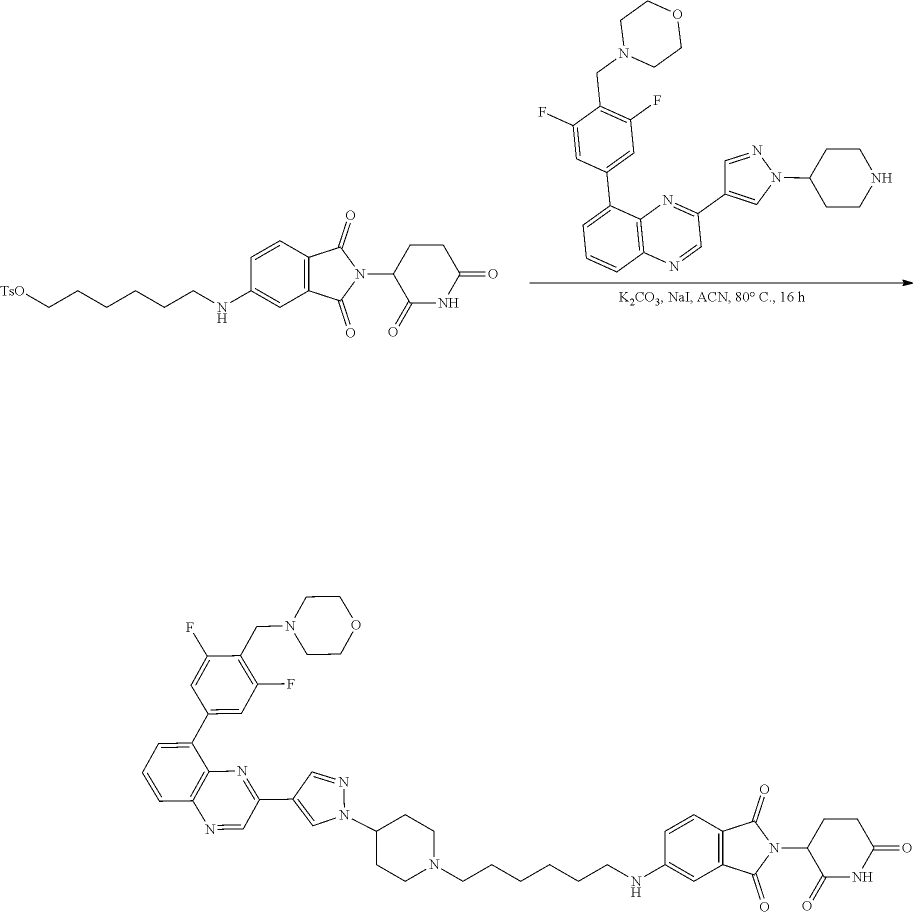 [SMILES: Cc1ccc(S(=O)(=O)OCCCCCCNc2ccc3c(c2)C(=O)N(C2CCC(=O)NC2=O)C3=O)cc1 ; Fc1cc(-c2cccc3ncc(-c4cnn(C5CCNCC5)c4)nc23)cc(F)c1CN1CCOCC1 ; O=C1CCC(N2C(=O)c3ccc(NCCCCCCN4CCC(n5cc(-c6cnc7cccc(-c8cc(F)c(CN9CCOCC9)c(F)c8)c7n6)cn5)CC4)cc3C2=O)C(=O)N1]

JA-266 was synthesized following the standard procedure for preparing JA-264 (6.8 mg, 32% yield) as a light yellow solid. MS (ESI) m/z: 846.4 [M+H]⁺.

### Example 355. 5-((6-(4-(4-(8-(3,5-Difluoro-4-(morpholinomethyl)phenyl)quinoxalin-2-yl)-1H-pyrazol-1-yl)piperidin-1-yl)hexyl)oxy)-2-(2,6-dioxopiperidin-3-yl)isoindoline-1,3-dione (JA-267)

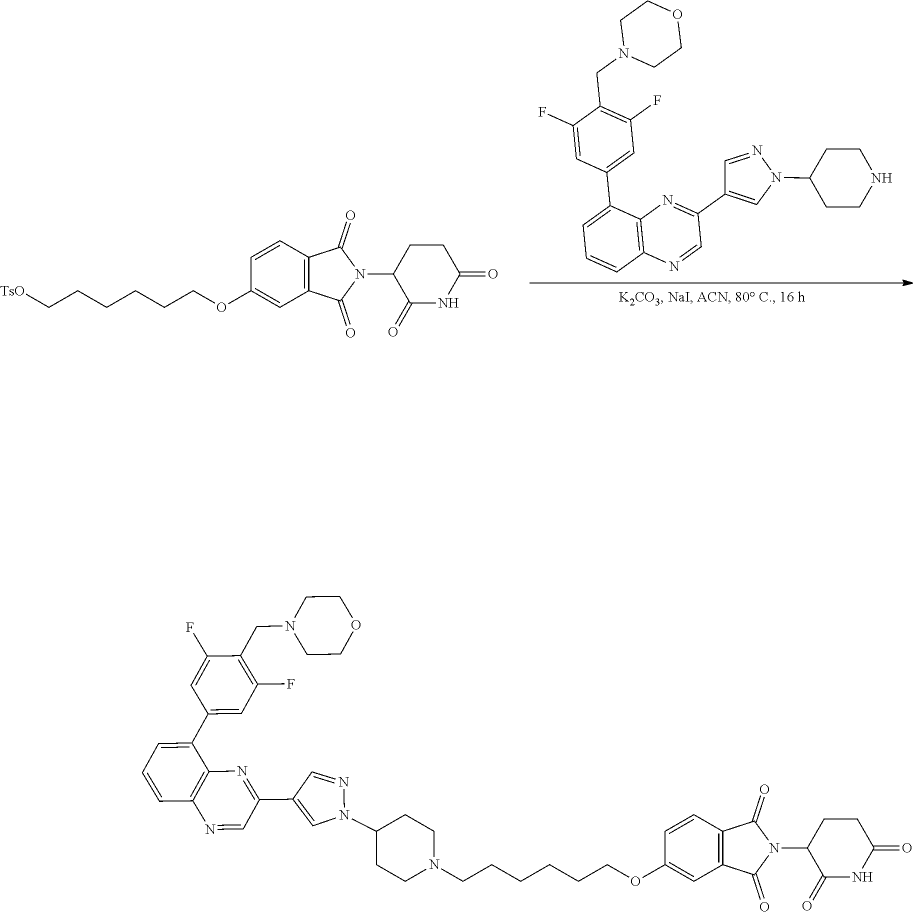 [SMILES: Cc1ccc(S(=O)(=O)OCCCCCCOc2ccc3c(c2)C(=O)N(C2CCC(=O)NC2=O)C3=O)cc1 ; Fc1cc(-c2cccc3ncc(-c4cnn(C5CCNCC5)c4)nc23)cc(F)c1CN1CCOCC1 ; O=C1CCC(N2C(=O)c3ccc(OCCCCCCN4CCC(n5cc(-c6cnc7cccc(-c8cc(F)c(CN9CCOCC9)c(F)c8)c7n6)cn5)CC4)cc3C2=O)C(=O)N1]

JA-267 was synthesized following the standard procedure for preparing JA-264 (9.2 mg, 38% yield) as a light yellow solid. MS (ESI) m/z: 847.4 [M+H]⁺.

### Example 356. 3-(6-(7-(4-(4-((5-(4-(Methylsulfonyl))phenyl)-[1,2,4]triazolo[1,5-a]pyridin-2-yl)amino)phenyl)piperazin-1-yl)-7-oxoheptyl)-1-oxoisoindolin-2-yl)piperidine-2,6-dione (JA-268)

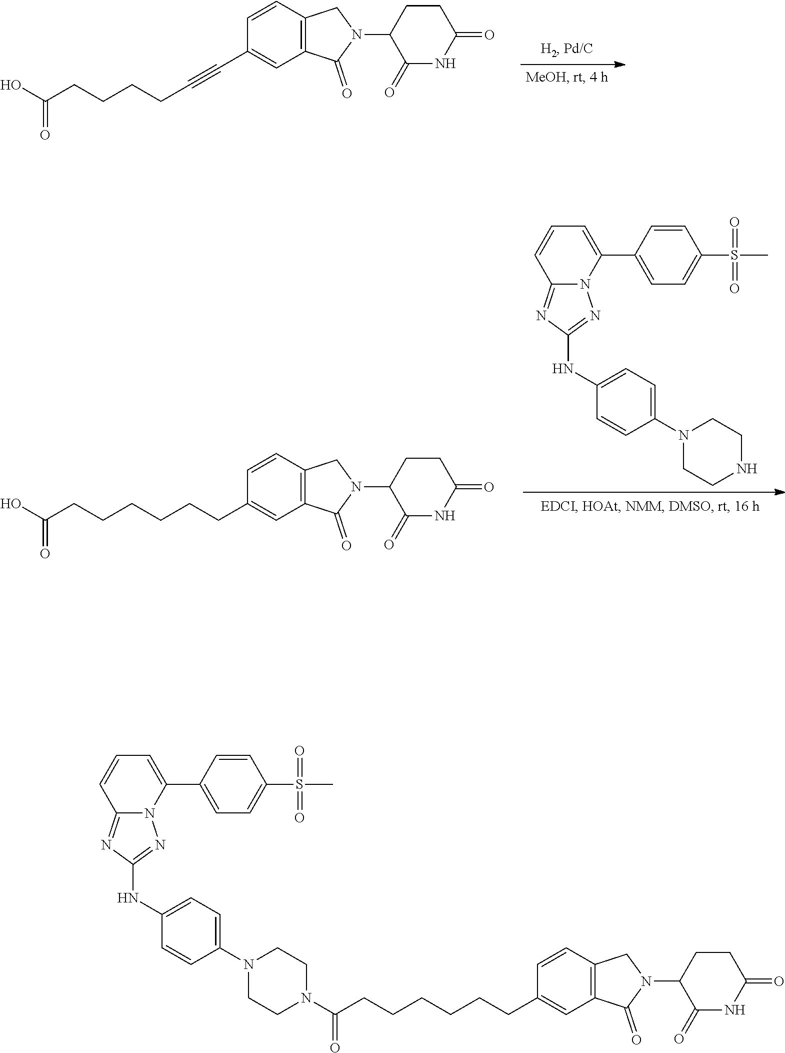 [SMILES: O=C(O)CCCCC#Cc1ccc2c(c1)C(=O)N(C1CCC(=O)NC1=O)C2 ; O=C(O)CCCCCCc1ccc2c(c1)C(=O)N(C1CCC(=O)NC1=O)C2 ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCCc6ccc7c(c6)C(=O)N(C6CCC(=O)NC6=O)C7)CC5)cc4)nn23)cc1]

### Step 1: Synthesis of 7-(2-(2,6-dioxopiperidin-3-yl)-3-oxoisoindolin-5-yl)heptanoic acid

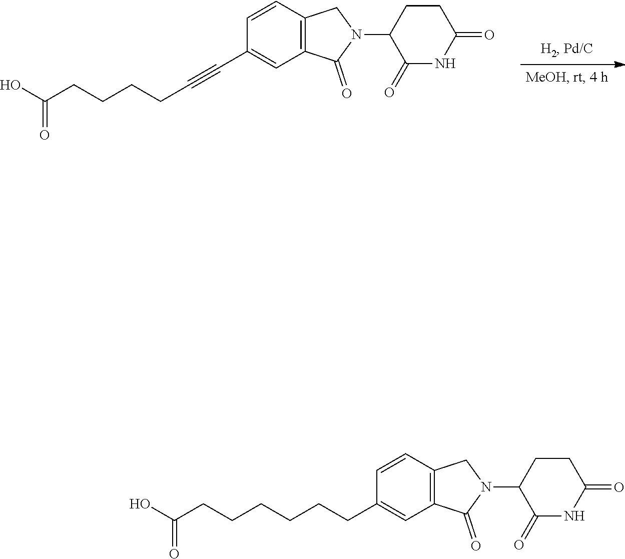 [SMILES: O=C(O)CCCCC#Cc1ccc2c(c1)C(=O)N(C1CCC(=O)NC1=O)C2 ; O=C(O)CCCCCCc1ccc2c(c1)C(=O)N(C1CCC(=O)NC1=O)C2]

To a solution of 7-(2-(2,6-dioxopiperidin-3-yl)-3-oxoisoindolin-5-yl)hept-6-ynoic acid (100 mg, 261 umol) in MeOH (10 mL) was added Pd/C (5.5 mg, 2.6 umol). After the reaction was stirred at 25° C. for 16 h, the reaction mixture was purified by reverse-phase chromatography to give the desired product (77 mg, 78.4% yield) as a light yellow solid. MS (ESI) m/z: 371.2 [M−H]⁻.

### Step 2: Synthesis of 3-(6-(7-(4-(4-((5-(4-(methylsulfonyl)phenyl)-[1,2,4]triazolo[1,5-a]pyridin-2-yl)amino)phenyl)piperazin-1-yl)-7-oxoheptyl)-1-oxoisoindolin-2-yl)piperidine-2,6-dione

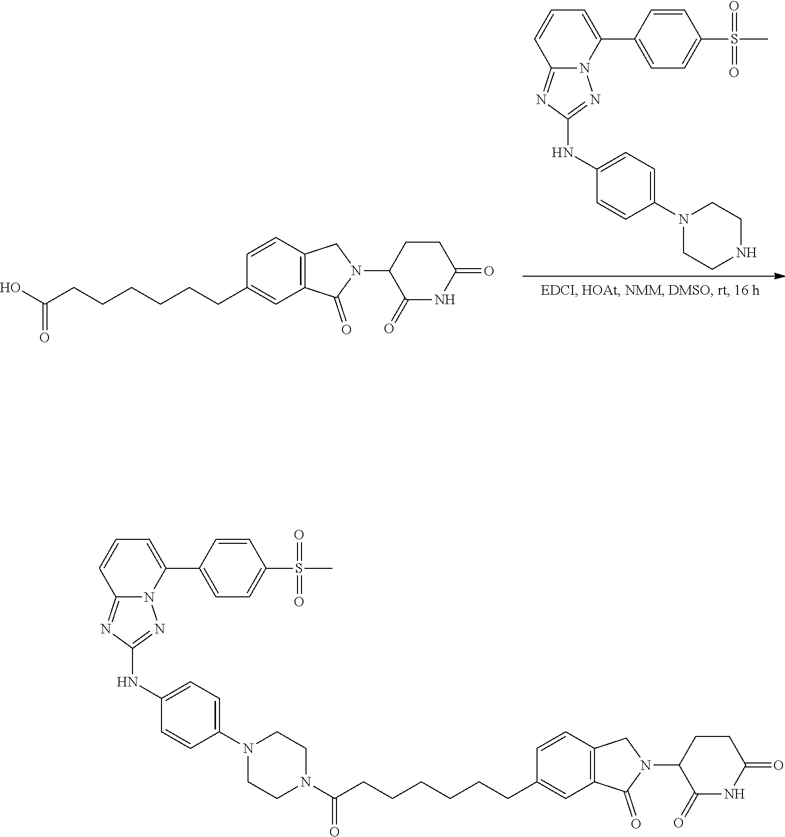 [SMILES: O=C(O)CCCCCCc1ccc2c(c1)C(=O)N(C1CCC(=O)NC1=O)C2 ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCCc6ccc7c(c6)C(=O)N(C6CCC(=O)NC6=O)C7)CC5)cc4)nn23)cc1]

JA-268 was synthesized following the standard procedure for preparing JA-001 (11 mg, 42% yield) as a light yellow solid. MS (ESI) m/z: 803.3 [M+H]⁺.

### Example 357. 3-(6-(7-(4-(4-(8-(3,5-Difluoro-4-(morpholinomethyl)phenyl)quinoxalin-2-yl)-1H-pyrazol-1-yl)piperidin-1-yl)-7-oxoheptyl)-1-oxoisoindolin-2-yl)piperidine-2,6-dione (JA-269)

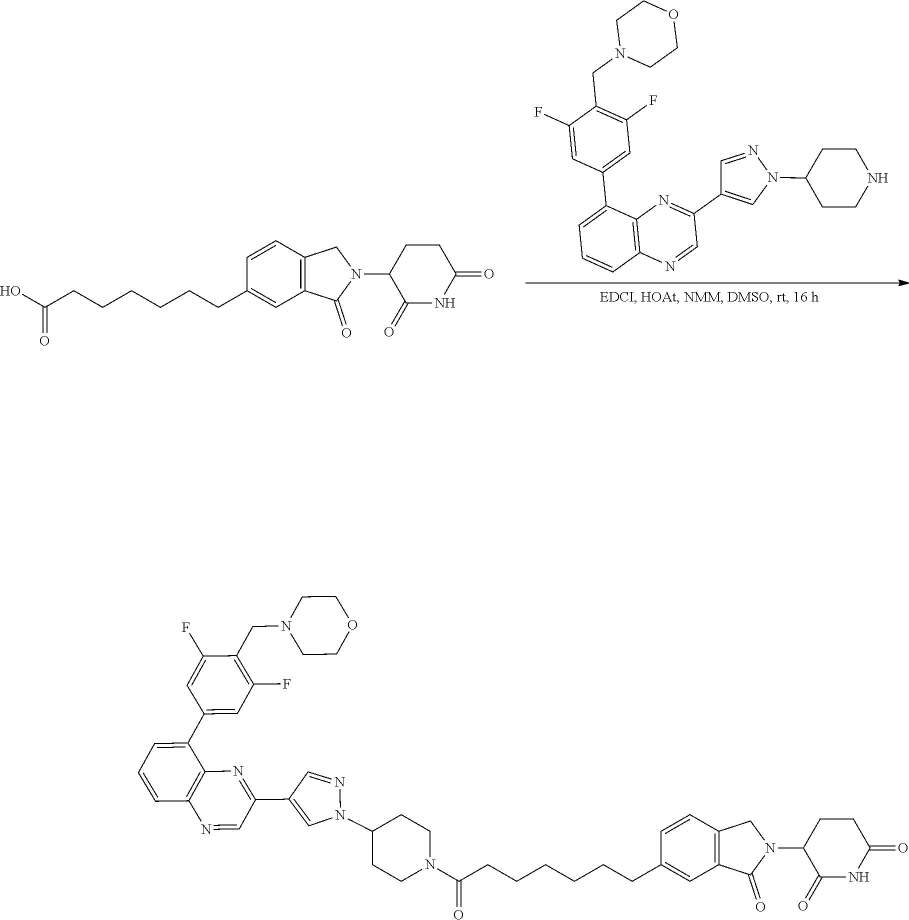 [SMILES: O=C(O)CCCCCCc1ccc2c(c1)C(=O)N(C1CCC(=O)NC1=O)C2 ; Fc1cc(-c2cccc3ncc(-c4cnn(C5CCNCC5)c4)nc23)cc(F)c1CN1CCOCC1 ; O=C1CCC(N2Cc3ccc(CCCCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-269 was synthesized following the standard procedure for preparing JA-001 (7.2 mg, 35% yield) as a light yellow solid. MS (ESI) m/z: 845.4 [M+H]⁺.

### Example 358. 3-(5-(7-(4-(4-(8-(3,5-Difluoro-4-(morpholinomethyl)phenyl)quinoxalin-2-yl)-1H-pyrazol-1-yl)piperidin-1-yl)-7-oxoheptyl)-1-oxoisoindolin-2-yl)piperidine-2,6-dione (JA-270)

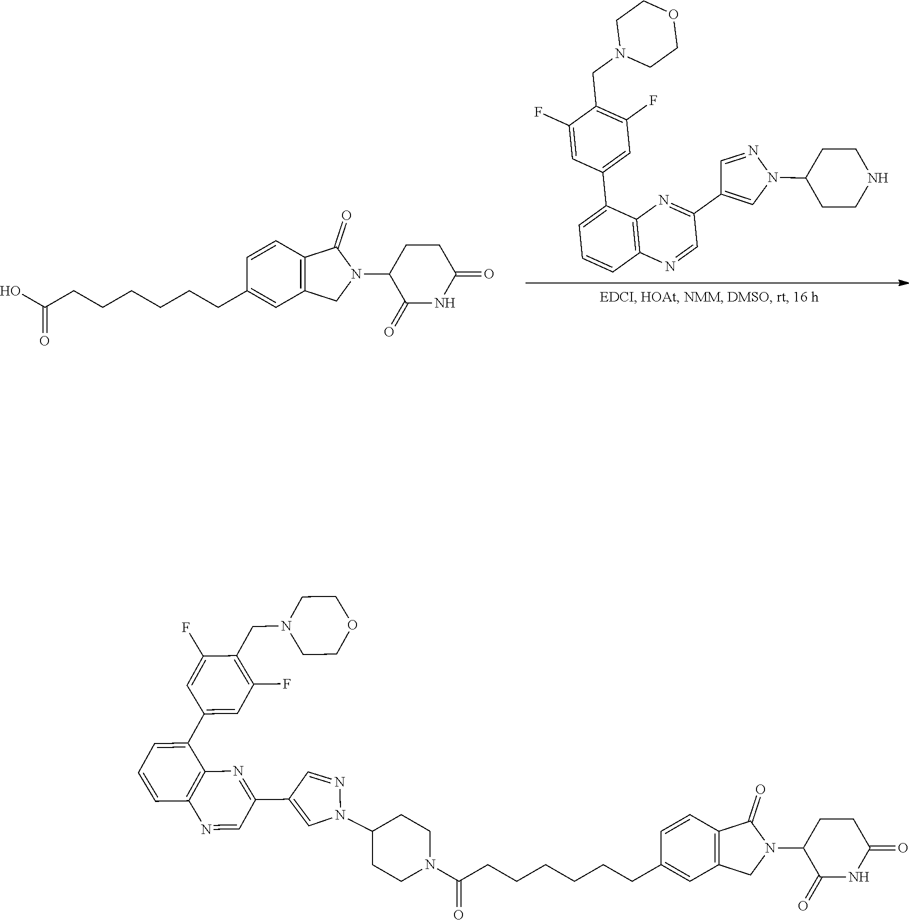 [SMILES: O=C(O)CCCCCCc1ccc2c(c1)CN(C1CCC(=O)NC1=O)C2=O ; Fc1cc(-c2cccc3ncc(-c4cnn(C5CCNCC5)c4)nc23)cc(F)c1CN1CCOCC1 ; O=C1CCC(N2Cc3cc(CCCCCCC(=O)N4CCC(n5cc(-c6cnc7cccc(-c8cc(F)c(CN9CCOCC9)c(F)c8)c7n6)cn5)CC4)ccc3C2=O)C(=O)N1]

JA-270 was synthesized following the standard procedure for preparing JA-001 (8.7 mg, 37% yield) as a light yellow solid. MS (ESI) m/z: 845.4 [M+H]⁺.

### Example 359. 3-(5-(7-(4-(4-(8-(3,5-Difluoro-4-(morpholinomethyl)phenyl)quinoxalin-2-yl)-1H-pyrazol-1-yl)piperidin-1-yl)-7-oxohept-1-yn-1-yl)-1-oxoisoindolin-2-yl)piperidine-2,6-dione (JA-271)

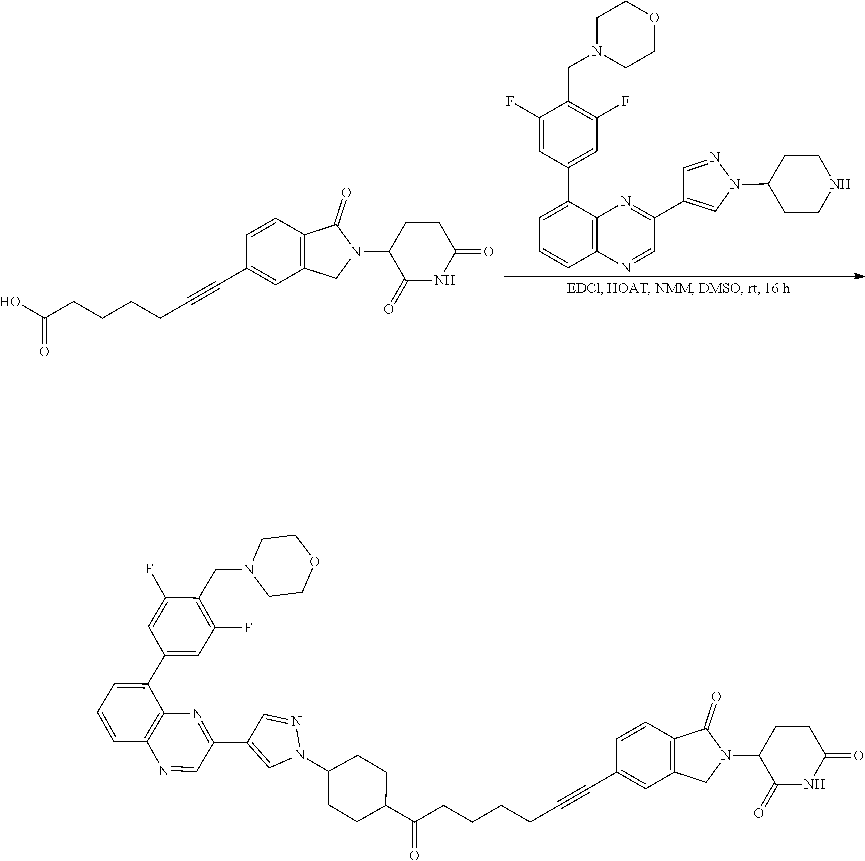 [SMILES: O=C(O)CCCCC#Cc1ccc2c(c1)CN(C1CCC(=O)NC1=O)C2=O ; Fc1cc(-c2cccc3ncc(-c4cnn(C5CCNCC5)c4)nc23)cc(F)c1CN1CCOCC1 ; O=C1CCC(N2Cc3cc(C#CCCCCC(=O)C4CCC(n5cc(-c6cnc7cccc(-c8cc(F)c(CN9CCOCC9)c(F)c8)c7n6)cn5)CC4)ccc3C2=O)C(=O)N1]

JA-271 was synthesized following the standard procedure for preparing JA-001 (9.3 mg, 39% yield) as a light yellow solid. MS (ESI) m/z: 841.4 [M+H]⁺.

### Example 360. 3-(6-((6-(4-(4-(8-(3,5-Difluoro-4-(morpholinomethyl)phenyl)quinoxalin-2-yl)-1H-pyrazol-1-yl)piperidin-1-yl)-6-oxohexyl)amino)-1-oxoisoindolin-2-yl)piperidine-2,6-dione (JA-272)

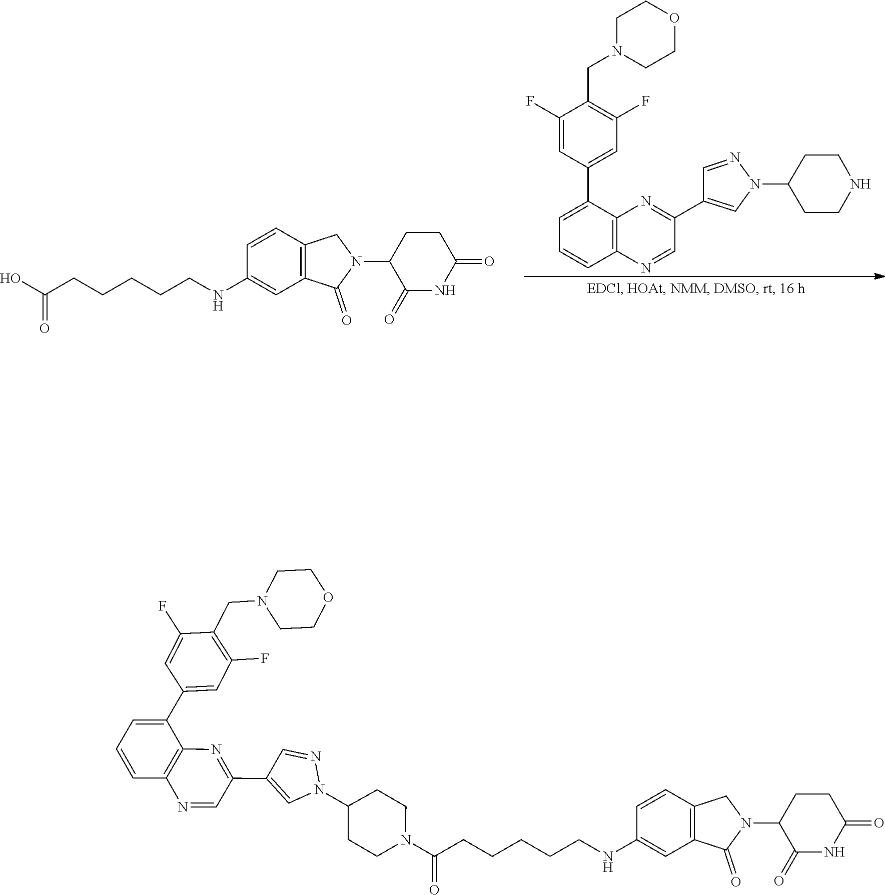 [SMILES: O=C(O)CCCCCNc1ccc2c(c1)C(=O)N(C1CCC(=O)NC1=O)C2 ; Fc1cc(-c2cccc3ncc(-c4cnn(C5CCNCC5)c4)nc23)cc(F)c1CN1CCOCC1 ; O=C1CCC(N2Cc3ccc(NCCCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-272 was synthesized following the standard procedure for preparing JA-001 (7.8 mg, 34% yield) as a light yellow solid. MS (ESI) m/z: 846.4 [M+H]⁺.

### Example 361. 3-(5-((6-(4-(4-(8-(3,5-Difluoro-4-(morpholinomethyl)phenyl)quinoxalin-2-yl)-1H-pyrazol-1-yl)piperidin-1-yl)-6-oxohexyl)amino)-1-oxoisoindolin-2-yl)piperidine-2,6-dione (JA-273)

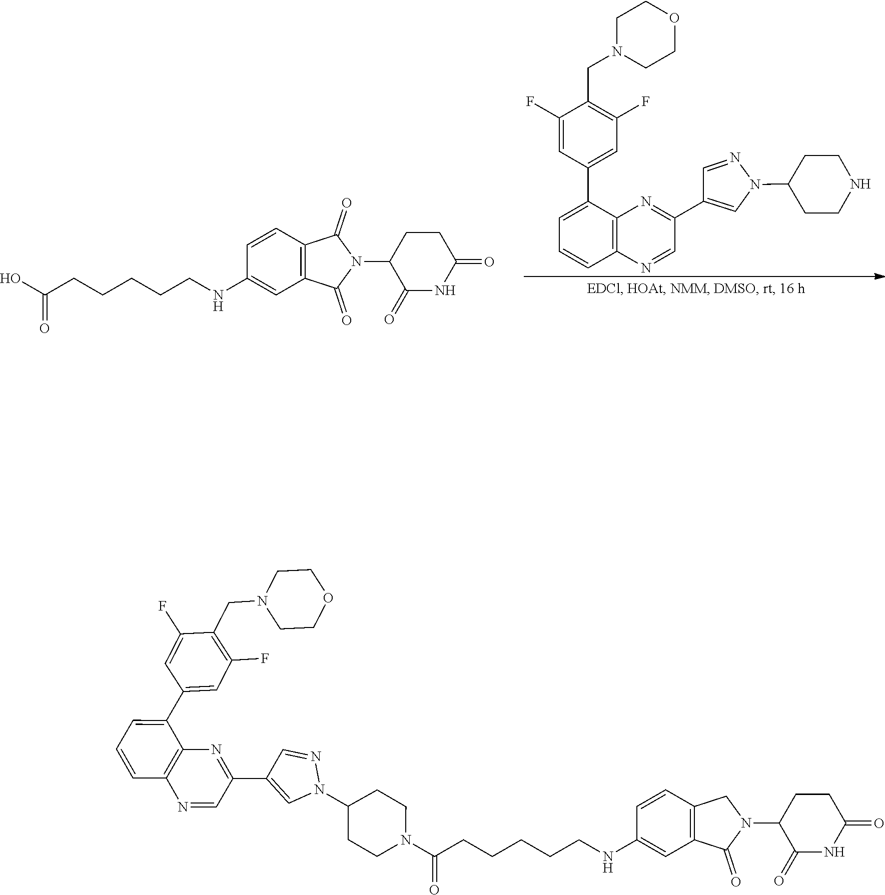 [SMILES: O=C(O)CCCCCNc1ccc2c(c1)C(=O)N(C1CCC(=O)NC1=O)C2=O ; Fc1cc(-c2cccc3ncc(-c4cnn(C5CCNCC5)c4)nc23)cc(F)c1CN1CCOCC1 ; O=C1CCC(N2Cc3ccc(NCCCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-273 was synthesized following the standard procedure for preparing JA-001 (9.1 mg, 37% yield) as a light yellow solid. MS (ESI) m/z: 846.4 [M+H]⁺.

### Example 362. 3-(6-((6-(4-(4-(8-(3,5-Difluoro-4-(morpholinomethyl)phenyl)quinoxalin-2-yl)-1H-pyrazol-1-yl)piperidin-1-yl)-6-oxohexyl)oxy)-1-oxoisoindolin-2-yl)piperidine-2,6-dione (JA-274)

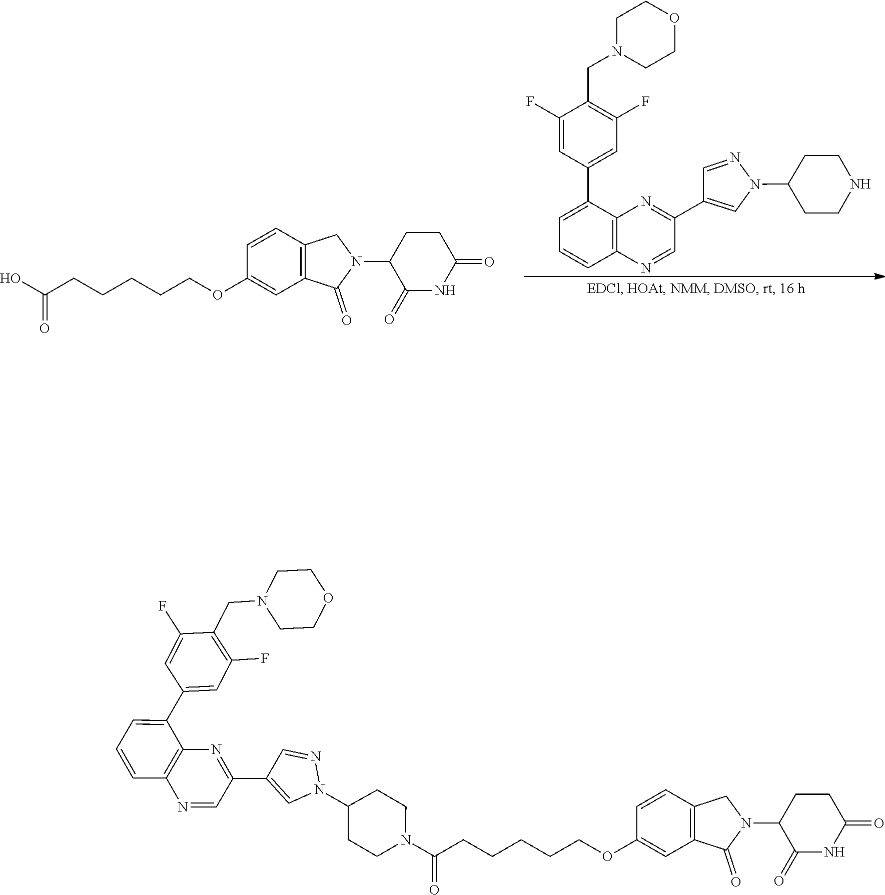 [SMILES: O=C(O)CCCCCOc1ccc2c(c1)C(=O)N(C1CCC(=O)NC1=O)C2 ; Fc1cc(-c2cccc3ncc(-c4cnn(C5CCNCC5)c4)nc23)cc(F)c1CN1CCOCC1 ; O=C1CCC(N2Cc3ccc(OCCCCCC(=O)N4CCC(n5cc(-c6cnc7cccc(-c8cc(F)c(CN9CCOCC9)c(F)c8)c7n6)cn5)CC4)cc3C2=O)C(=O)N1]

JA-274 was synthesized following the standard procedure for preparing JA-001 (8.5 mg, 35% yield) as a light yellow solid. MS (ESI) m/z: 847.4 [M+H]⁺.

### Example 363. 3-(5-((6-(4-(4-((5-(4-(Methylsulfonyl)phenyl)-[1,2,4]triazolo[1,5-a]pyridin-2-yl)amino)phenyl)piperazin-1-yl)-6-oxohexyl)amino)-1-oxoisoindolin-2-yl)piperidine-2,6-dione (JA-275)

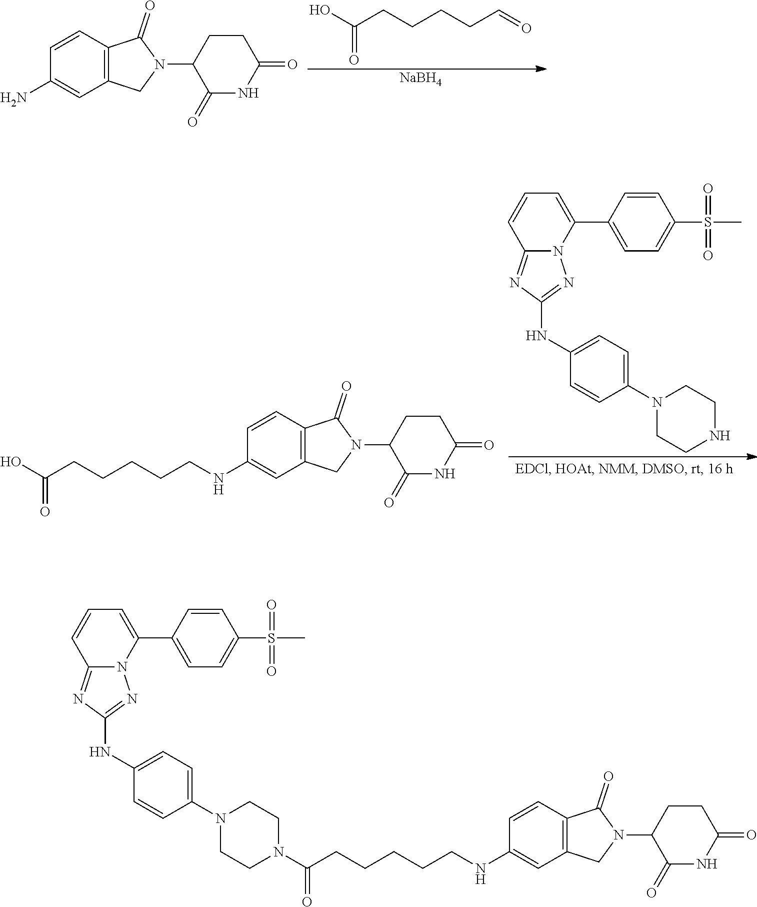 [SMILES: Nc1ccc2c(c1)CN(C1CCC(=O)NC1=O)C2=O ; O=CCCCCC(=O)O ; "NaBH4" ; O=C(O)CCCCCNc1ccc2c(c1)CN(C1CCC(=O)NC1=O)C2=O ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCNc6ccc7c(c6)CN(C6CCC(=O)NC6=O)C7=O)CC5)cc4)nn23)cc1]

### Step 1: Synthesis of 6-((2-(2,6-dioxopiperidin-3-yl)-1-oxoisoindolin-5-yl)amino)hexanoic acid

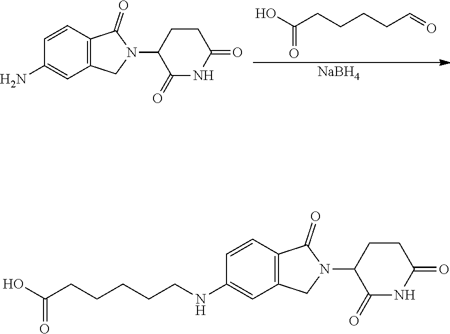

To a solution of 6-oxohexanoic acid (75 mg, 0.58 mmol) and 3-(5-amino-1-oxoisoindolin-2-yl)piperidine-2,6-dione (100 mg, 0.38 mmol) in DMF (6 mL) was added TMSCl (83 mg, 0.77 mmol) at 0° C. After the reaction was stirred at 0° C. for 30 min, NaBH₄ (44 mg, 1.16 mmol) was added to the mixture in two portions. After the reaction was stirred at 0° C. for 4 h, the reaction was poured into water (50 mL) and extracted with ethyl acetate (3×20 mL). The combined organic layers were washed with saturated brine (50 mL), dried over anhydrous sodium sulfate, filtered and evaporated under reduced pressure. The resulting residue was concentrated and purified by reverse-phase chromatography to give the desired product (23 mg, 10% yield) as a light yellow solid. MS (ESI) m/z: 372.2 [M−H]⁻.

### Step 2: Synthesis of 3-(5-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)-1-oxoisoindolin-2-yl)piperidine-2,6-dione

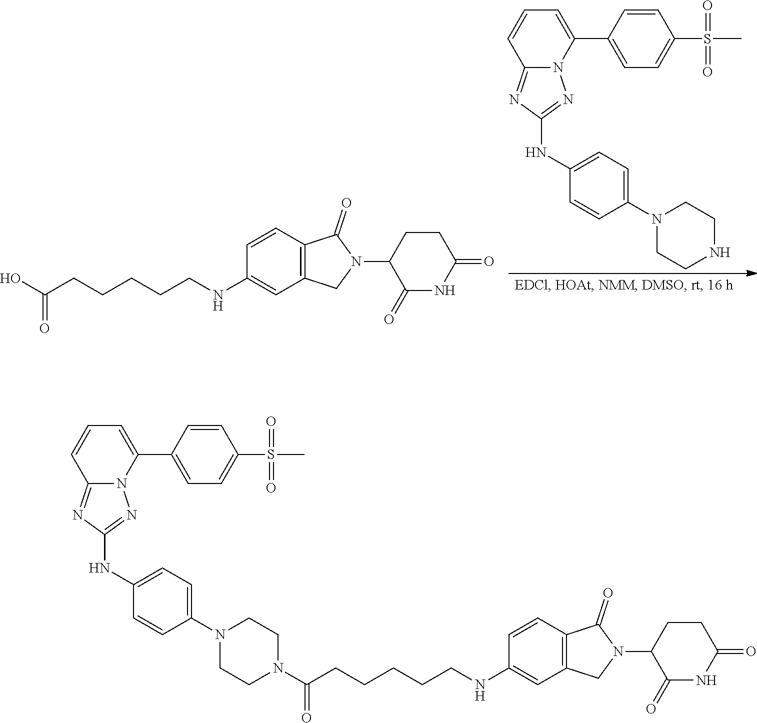 [SMILES: O=C(O)CCCCCNc1ccc2c(c1)CN(C1CCC(=O)NC1=O)C2=O ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCNc6ccc7c(c6)CN(C6CCC(=O)NC6=O)C7=O)CC5)cc4)nn23)cc1]

JA-275 was synthesized following the standard procedure for preparing JA-001 (9.8 mg, 38% yield) as a light yellow solid. MS (ESI) m/z: 804.3 [M+H]⁺.

### Example 364. 3-(6-((6-(4-(4-((5-(4-(Methylsulfonyl)phenyl)-[1,2,4]triazolo[1,5-a]pyridin-2-yl)amino)phenyl)piperazin-1-yl)-6-oxohexyl)amino)-1-oxoisoindolin-2-yl)piperidine-2,6-dione (JA-276)

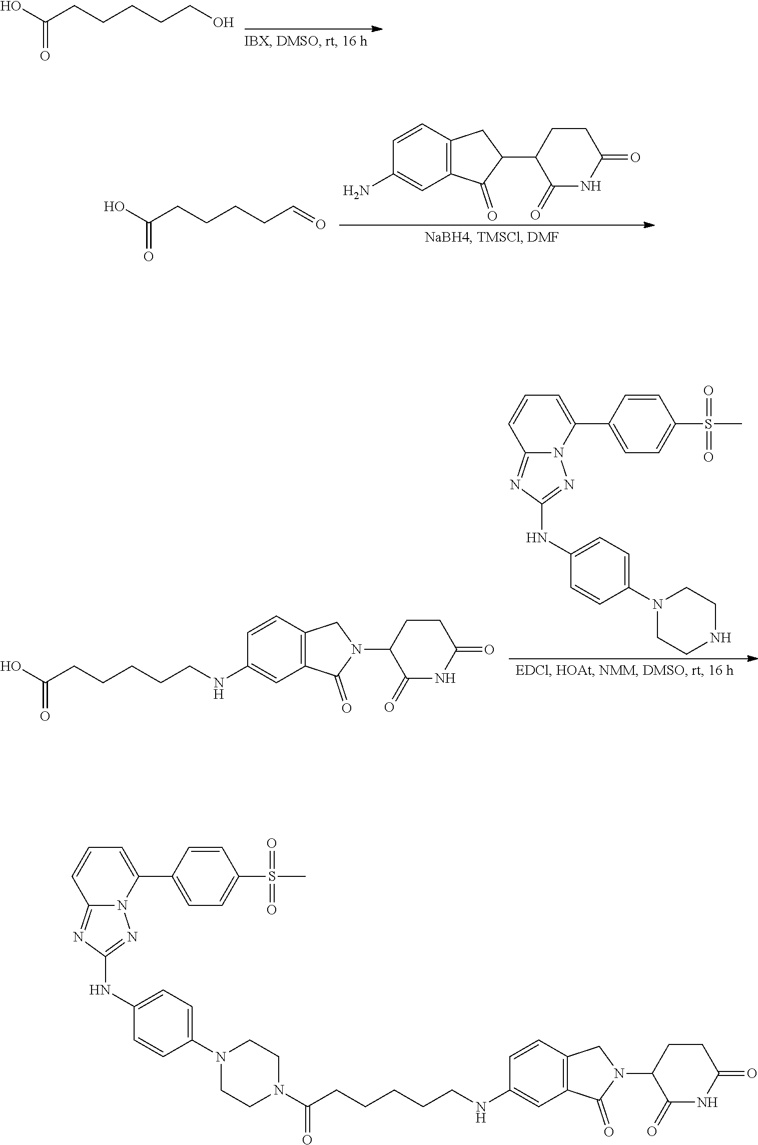 [SMILES: O=C(O)CCCCCO ; O=CCCCCC(=O)O ; Nc1ccc2c(c1)C(=O)C(C1CCC(=O)NC1=O)C2 ; O=C(O)CCCCCNc1ccc2c(c1)C(=O)N(C1CCC(=O)NC1=O)C2 ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCNc6ccc7c(c6)C(=O)N(C6CCC(=O)NC6=O)C7)CC5)cc4)nn23)cc1]

### Step 1: Synthesis of 6-oxohexanoic acid

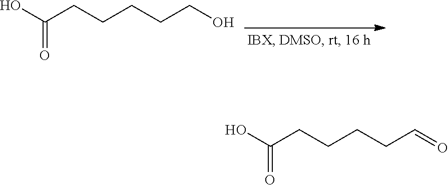 [SMILES: O=C(O)CCCCCO ; O=CCCCCC(=O)O]

To a solution of 6-hydroxyhexanoic acid (500 mg, 3.8 mmol) in DMSO (10 mL) was added IBX (2.1 g, 7.6 mmol). After the reaction was stirred at room temperature for 16 h, the reaction was poured into water (50 mL) and extracted with ethyl acetate (3×20 mL). The combined organic layers were washed with saturated brine (50 mL), dried over anhydrous sodium sulfate, filtered and evaporated under reduced pressure. The crude product (486 mg, 98% yield) was used directly in the next step. MS (ESI) m/z: 129.1 [M−H]⁻.

### Step 2: Synthesis of 6-((2-(2,6-dioxopiperidin-3-yl)-3-oxoisoindolin-5-yl)amino)hexanoic acid

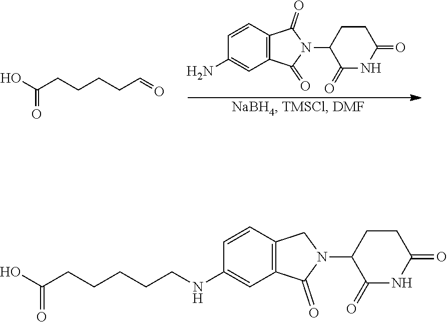

To a solution of 6-oxohexanoic acid (75 mg, 0.58 mmol) and 3-(6-amino-1-oxoisoindolin-2-yl)piperidine-2,6-dione (100 mg, 0.38 mmol) in DMF (6 mL) was added TMSCl (83 mg, 0.77 mmol) at 0° C. After the reaction was stirred at 0° C. for 30 min, NaBH₄ (44 mg, 1.16 mmol) was added to the mixture. After the resulting reaction mixture was stirred at 0° C. for 4 h, the reaction mixture was poured into water (50 mL) and extracted with ethyl acetate (3×20 mL). The combined organic layers were washed with saturated brine (50 mL), dried over anhydrous sodium sulfate, filtered and evaporated under reduced pressure. The resulting residue was purified by reverse-phase chromatography to give the desired product (18 mg, 8% yield) as a light yellow solid. MS (ESI) m/z: 372.2 [M−H]⁻.

### Step 3: Synthesis of 3-(6-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)-1-oxoisoindolin-2-yl)piperidine-2,6-dione

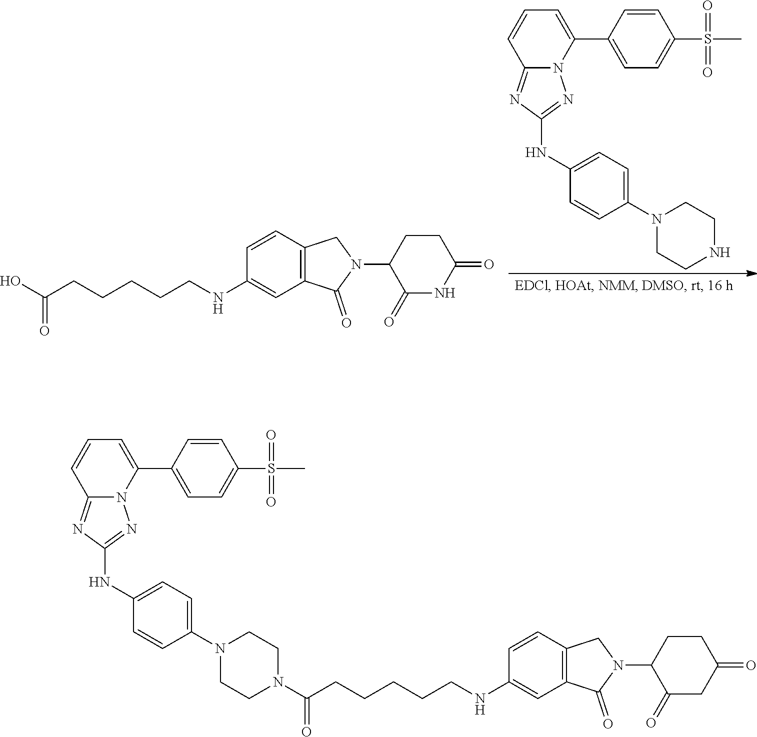 [SMILES: O=C(O)CCCCCNc1ccc2c(c1)C(=O)N(C1CCC(=O)NC1=O)C2 ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCNc6ccc7c(c6)C(=O)N(C6CCC(=O)CC6=O)C7)CC5)cc4)nn23)cc1]

JA-276 was synthesized following the standard procedure for preparing JA-001 (11 mg, 42% yield) as a light yellow solid. MS (ESI) m/z: 804.3 [M+H]⁺.

### Example 365. 3-(6-((6-(4-(4-((5-(4-(Methylsulfonyl)phenyl)-[1,2,4]triazolo[1,5-a]pyridin-2-yl)amino)phenyl)piperazin-1-yl)-6-oxohexyl)oxy)-1-oxoisoindolin-2-yl)piperidine-2,6-dione (JA-277)

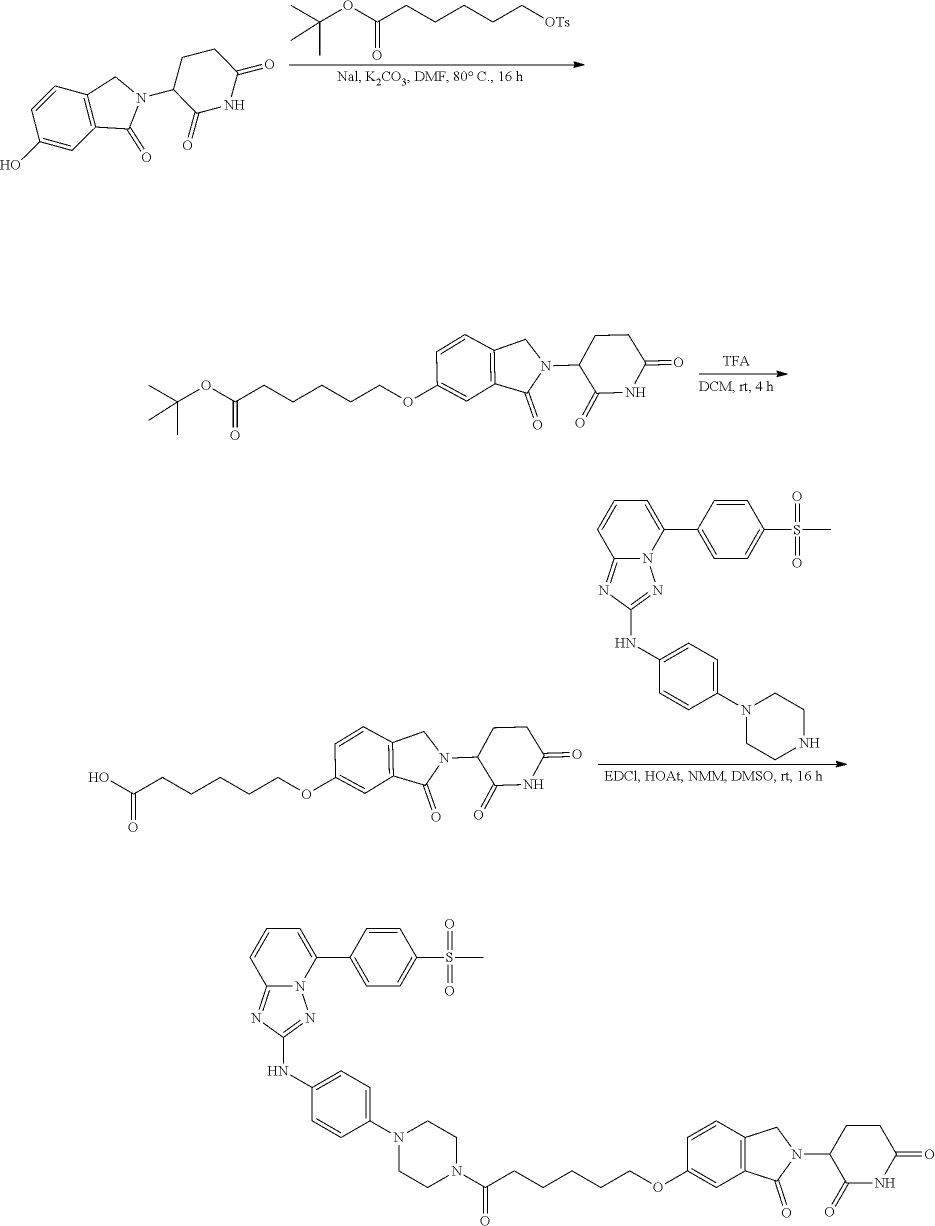

### Step 1: Synthesis of tert-butyl 6-((2-(2,6-dioxopiperidin-3-yl)-3-oxoisoindolin-5-yl)oxy)hexanoate

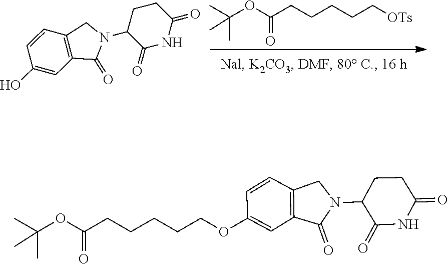

A mixture of 3-(6-hydroxy-1-oxoisoindolin-2-yl)piperidine-2,6-dione (100 mg, 385 umol), K₂CO₃ (106 mg, 769 umol), NaI (22 mg, 385 umol), tert-butyl 6-(tosyloxy)hexanoate (263 mg, 769 umol) and DMF (10 mL) was stirred at 80° C. for 16 h. The reaction mixture was purified by reverse-phase chromatography to give the desired product (35 mg, 23% yield) as a light yellow solid. MS (ESI) m/z: 431.2 [M+H]⁺.

### Step 2: Synthesis of 6-((2-(2,6-dioxopiperidin-3-yl)-3-oxoisoindolin-5-yl)oxy)hexanoic acid

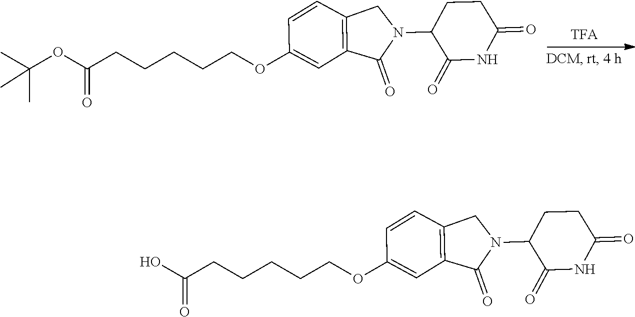 [SMILES: CC(C)(C)OC(=O)CCCCCOc1ccc2c(c1)C(=O)N(C1CCC(=O)NC1=O)C2 ; O=C(O)CCCCCOc1ccc2c(c1)C(=O)N(C1CCC(=O)NC1=O)C2]

To a solution of tert-butyl 6-((2-(2,6-dioxopiperidin-3-yl)-3-oxoisoindolin-5-yl)oxy)hexanoate (30 mg, 69.7 umol) in DCM (5 mL) was added TFA (4 mL), before it was stirred at room temperature for 2 h. The mixture was concentrated and purified by reverse-phase chromatography to give the desired product (25 mg, 90% yield) as a light yellow solid. MS (ESI) m/z: 373.1 [M−H]⁻.

### Step 3: Synthesis of 3-(6-((6-(4-(4-((5-(4-(methylsulfonyl)phenyl)-[1,2,4]triazolo[1,5-a]pyridin-2-yl)amino)phenyl)piperazin-1-yl)-6-oxohexyl)oxy)-1-oxoisoindolin-2-yl)piperidine-2,6-dione

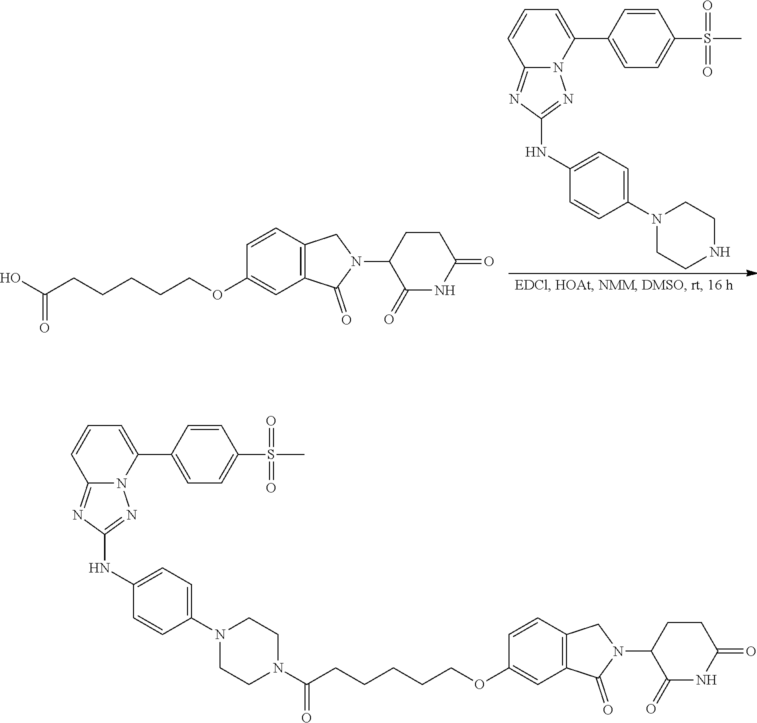 [SMILES: O=C(O)CCCCCOc1ccc2c(c1)C(=O)N(C1CCC(=O)NC1=O)C2 ; CS(=O)(=O)c1ccc(-c2cccc3nc(Nc4ccc(N5CCNCC5)cc4)nn23)cc1 ; CS(=O)(=O)c1ccc(-c2cccc3nc(Nc4ccc(N5CCN(C(=O)CCCCCOc6ccc7c(c6)C(=O)N(C6CCC(=O)NC6=O)C7)CC5)cc4)nn23)cc1]

JA-277 was synthesized following the standard procedure for preparing JA-001 (11 mg, 42% yield) as a light yellow solid. MS (ESI) m/z: 805.3 [M+H]⁺.

### Example 366. 3-(3-(6-(4-(4-(8-(3,5-Difluoro-4-(morpholinomethyl)phenyl)quinoxalin-2-yl)-1H-pyrazol-1-yl)piperidin-1-yl)-6-oxohexyl)-2-oxo-2,3-dihydro-1H-benzo[d]imidazol-1-yl)piperidine-2,6-dione (JA-278)

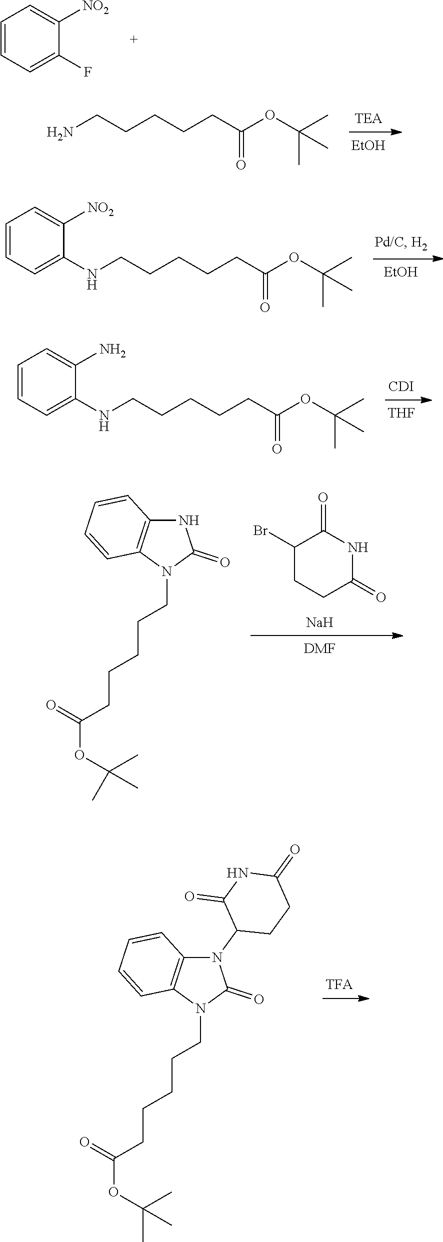

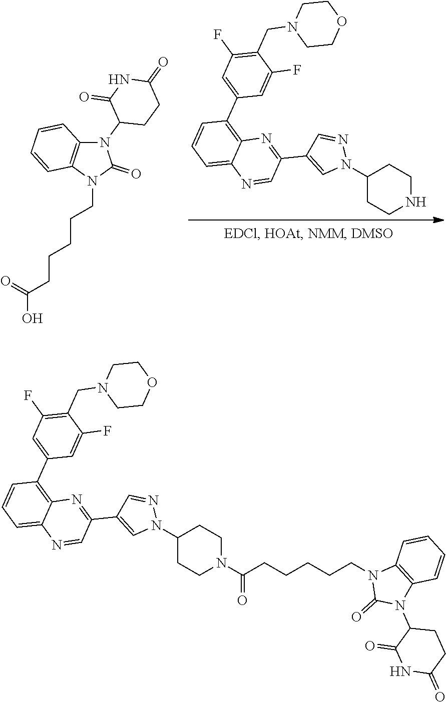 [SMILES: O=C(O)CCCCCn1c(=O)n(C2CCC(=O)NC2=O)c2ccccc21 ; Fc1cc(-c2cccc3ncc(-c4cnn(C5CCNCC5)c4)nc23)cc(F)c1CN1CCOCC1 ; O=C1CCC(n2c(=O)n(CCCCCC(=O)N3CCC(n4cc(-c5cnc6cccc(-c7cc(F)c(CN8CCOCC8)c(F)c7)c6n5)cn4)CC3)c3ccccc32)C(=O)N1]

### Step 1: Synthesis of tert-butyl 6-((2-nitrophenyl)amino)hexanoate

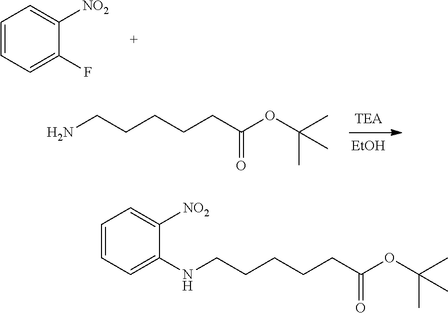

A mixture of 1-fluoro-2-nitrobenzene (528 mg, 3.74 mmol), tert-butyl 6-aminohexanoate (841 mg, 4.49 mmol) and TEA (1.14 g, 11.23 mmol) in EtOH (10 mL) was stirred at 85° C. overnight. The reaction mixture was concentrated to afford the crude product which was used directly in the next step without further purification.

### Step 2: Synthesis of tert-butyl 6-((2-aminophenyl)amino)hexanoate

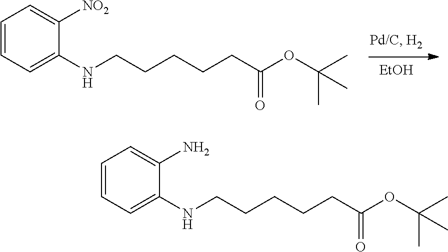 [SMILES: "EtOH" ; CC(C)(C)OC(=O)CCCCCNc1ccccc1[N+](=O)[O-] ; CC(C)(C)OC(=O)CCCCCNc1ccccc1N]

The mixture of tert-butyl 6-((2-nitrophenyl)amino)hexanoate (1.2 g, 3.89 mmol), Pd/C (100 mg) in EtOH (30 mL) was stirred under H₂ at room temperature for 1.5 h. After the reaction mixture was filtered, the filtrate was concentrated and the residue was purified by silica gel column chromatography (petroleum ether/EtOAc=8:1 to 5:1) to give desired product (690 mg, yield 64%). (ESI) m/z: 279.7 [M+H]⁺.

### Step 3: Synthesis of tert-butyl 6-(2-oxo-2,3-dihydro-1H-benzo[d]imidazol-1-yl)hexanoate

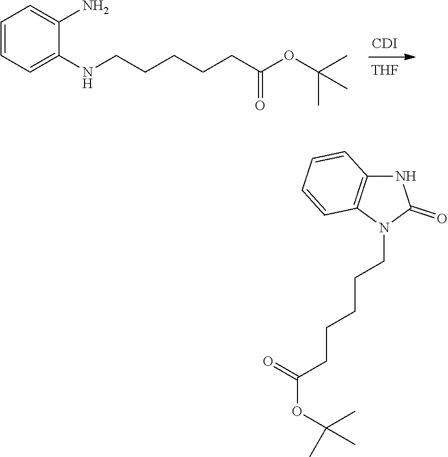

To a solution of tert-butyl 6-((2-aminophenyl)amino)hexanoate (592 mg, 2.13 mmol) in THF (40 mL) was added CDI (517 mg, 3.19 mmol). After the resulting mixture was stirred at room temperature for 16 h, the reaction mixture was concentrated and diluted with EtOAc, washed with water. The organic layer was dried over Na₂SO₄, filtered and concentrated. The residue was purified by silica gel column chromatography (petroleum ether/EtOAc=3:1 to 2:1) to give desired product (579 mg, yield 89%). (ESI) m/z: 305.5 [M+H]⁺.

### Step 4: Synthesis of tert-butyl 6-(3-(2,6-dioxopiperidin-3-yl)-2-oxo-2,3-dihydro-1H-benzo[d]imidazol-1-yl)hexanoate

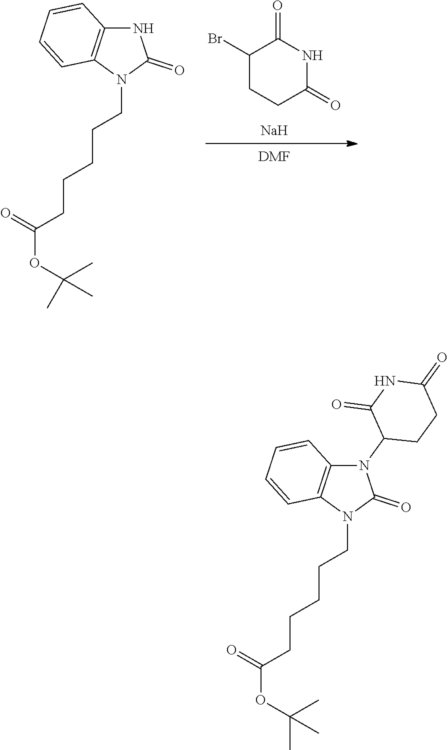

To a solution of tert-butyl 6-(2-oxo-2,3-dihydro-1H-benzo[d]imidazol-1-yl)hexanoate (550 mg, 1.81 mmol) in anhydrous DMF (2.2 ml) was added NaH (56 mg, 2.35 mmol) at 0° C. under N₂. The resulting mixture was stirred at 0° C. for 15 min, before it was added dropwise a solution of 3-bromopiperidine-2,6-dione (173 mg, 0.90 mmol) in anhydrous DMF (2.2 mL) over 10 min. The reaction mixture was stirred at room temperature overnight, before it was quenched with water and extracted with EtOAc. The aqeuous phase was adjusted to pH=5˜6 with citric acid and extracted with EtOAc. The combined organic layers were dried over Na₂SO₄, filtered and concentrated. The residue was purified by silica gel column chromatography (petroleum ether/EtOAc=2:1 to 1:1) to give desired product (170 mg, yield 23%). (ESI) m/z: 416.6 [M+H]⁺.

### Step 5: Synthesis of 6-(3-(2,6-dioxopiperidin-3-yl)-2-oxo-2,3-dihydro-1H-benzo[d]imidazol-1-yl)hexanoic acid

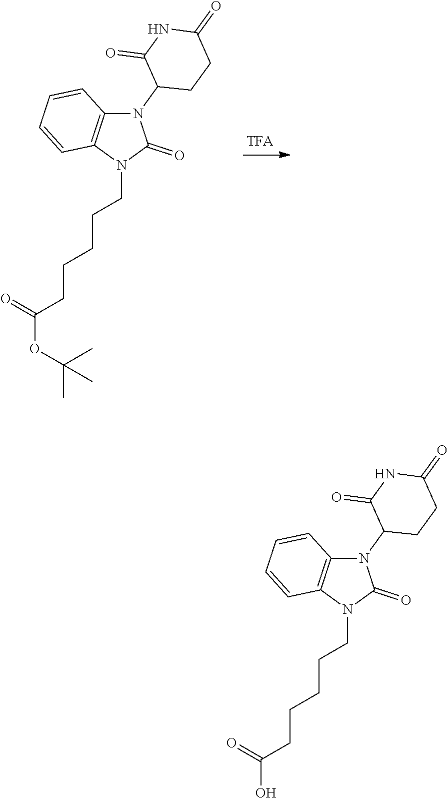 [SMILES: CC(C)(C)OC(=O)CCCCCn1c(=O)n(C2CCC(=O)NC2=O)c2ccccc21 ; O=C(O)CCCCCn1c(=O)n(C2CCC(=O)NC2=O)c2ccccc21]

A mixture of tert-butyl 6-(3-(2,6-dioxopiperidin-3-yl)-2-oxo-2,3-dihydro-1H-benzo[d]imidazol-1-yl)hexanoate (170 mg, 0.41 mmol) in DCM (3 mL) and TFA (3 mL) was stirred at room temperature for 0.5 h. The solvent was removed to afford the desired product (147 mg, yield 99%). (ESI) m/z: 360.6 [M+H]⁺.

### Step 6: Synthesis of 3-(3-(6-(4-(4-(8-(3,5-difluoro-4-(morpholinomethyl)phenyl)quinoxalin-2-yl)-1H-pyrazol-1-yl)piperidin-1-yl)-6-oxohexyl)-2-oxo-2,3-dihydro-1H-benzo[d]imidazol-1-yl)piperidine-2,6-dione (JA-278)

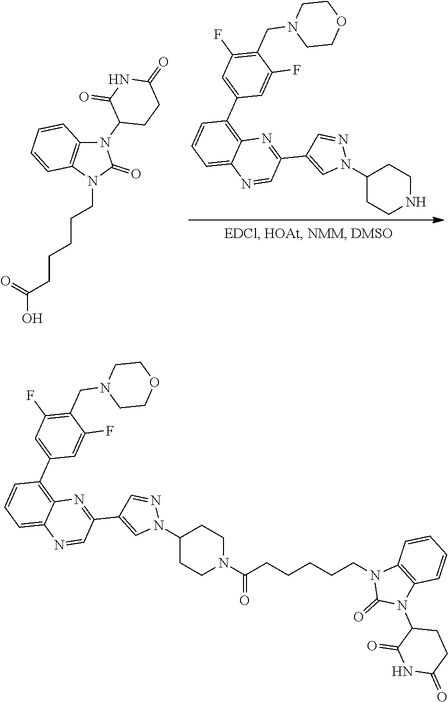 [SMILES: O=C(O)CCCCCn1c(=O)n(C2CCC(=O)NC2=O)c2ccccc21 ; Fc1cc(-c2cccc3ncc(-c4cnn(C5CCNCC5)c4)nc23)cc(F)c1CN1CCOCC1 ; O=C1CCC(n2c(=O)n(CCCCCC(=O)N3CCC(n4cc(-c5cnc6cccc(-c7cc(F)c(CN8CCOCC8)c(F)c7)c6n5)cn4)CC3)c3ccccc32)C(=O)N1]

JA-278 was synthesized following the standard procedure for preparing JA-001 (7.4 mg, yield 38%). MS (ESI) m/z: 833.1 [M+H]⁺.

### Example 367. 3-(3-(7-(4-(4-(8-(3,5-Difluoro-4-(morpholinomethyl)phenyl)quinoxalin-2-yl)-1H-pyrazol-1-yl)piperidin-1-yl)-7-oxoheptyl)-2-oxo-2,3-dihydro-1H-benzo[d]imidazol-1-yl)piperidine-2,6-dione (JA-279)

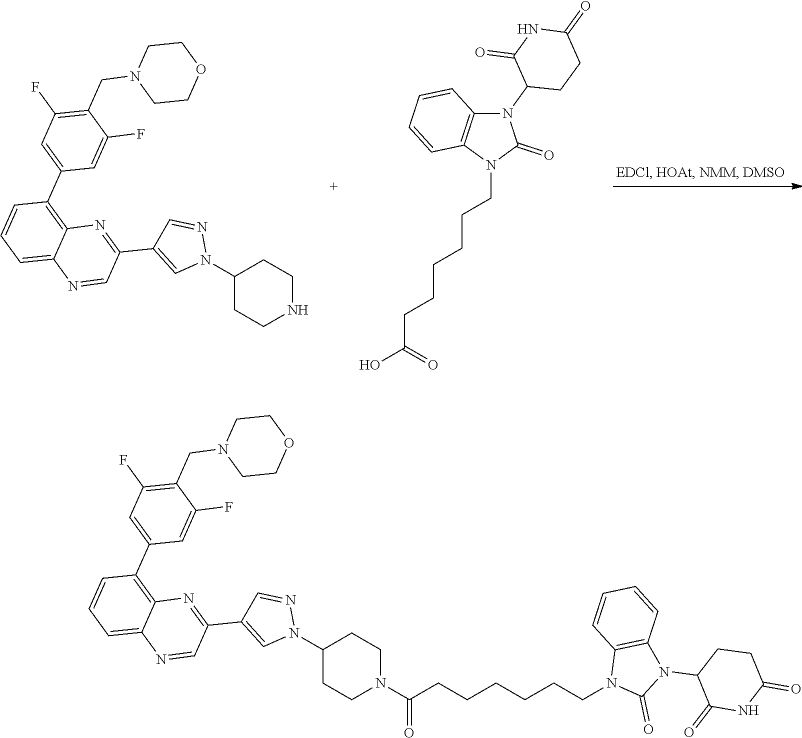 [SMILES: Fc1cc(-c2cccc3ncc(-c4cnn(C5CCNCC5)c4)nc23)cc(F)c1CN1CCOCC1 ; O=C(O)CCCCCCn1c(=O)n(C2CCC(=O)NC2=O)c2ccccc21 ; O=C1CCC(n2c(=O)n(CCCCCCC(=O)N3CCC(n4cc(-c5cnc6cccc(-c7cc(F)c(CN8CCOCC8)c(F)c7)c6n5)cn4)CC3)c3ccccc32)C(=O)N1]

JA-279 was synthesized following the standard procedure for preparing JA-278 (9.3 mg, 48% yield). MS (ESI) m/z: 847.1 [M+H]⁺.

### Example 368. 3-(3-(6-(4-(4-((5-(4-(Methylsulfonyl)phenyl)-[1,2,4]triazolo[1,5-a]pyridin-2-yl)amino)phenyl)piperazin-1-yl)-6-oxohexyl)-2-oxo-2,3-dihydro-1H-benzo[d]imidazol-1-yl)piperidine-2,6-dione (JA-280)

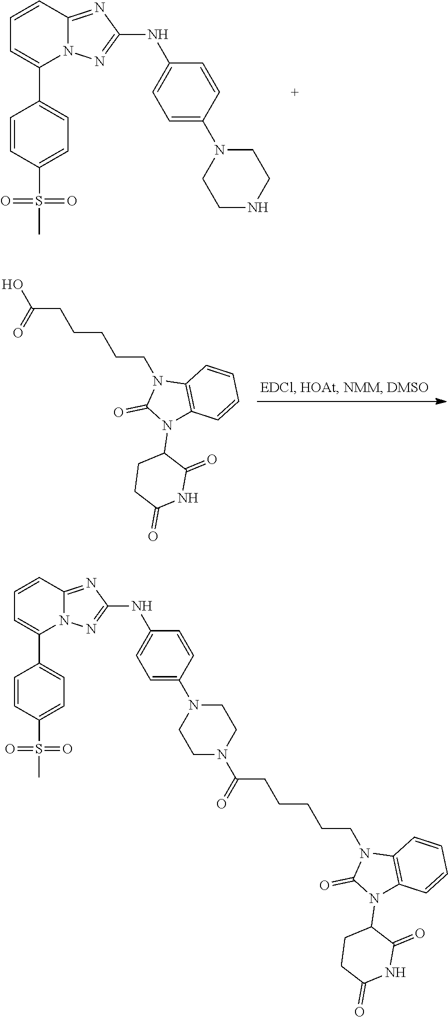 [SMILES: CS(=O)(=O)c1ccc(-c2cccc3nc(Nc4ccc(N5CCNCC5)cc4)nn23)cc1 ; O=C(O)CCCCCn1c(=O)n(C2CCC(=O)NC2=O)c2ccccc21 ; CS(=O)(=O)c1ccc(-c2cccc3nc(Nc4ccc(N5CCN(C(=O)CCCCCn6c(=O)n(C7CCC(=O)NC7=O)c7ccccc76)CC5)cc4)nn23)cc1]

JA-280 was synthesized following the standard procedure for preparing JA-278 (16 mg, 79% yield). MS (ESI) m/z: 791.0 [M+H]⁺.

### Example 369. 3-(3-(7-(4-(4-((5-(4-(Methylsulfonyl)phenyl)-[1,2,4]triazolo[1,5-a]pyridin-2-yl)amino)phenyl)piperazin-1-yl)-7-oxoheptyl)-2-oxo-2,3-dihydro-1H-benzo[d]imidazol-1-yl)piperidine-2,6-dione (JA-281)

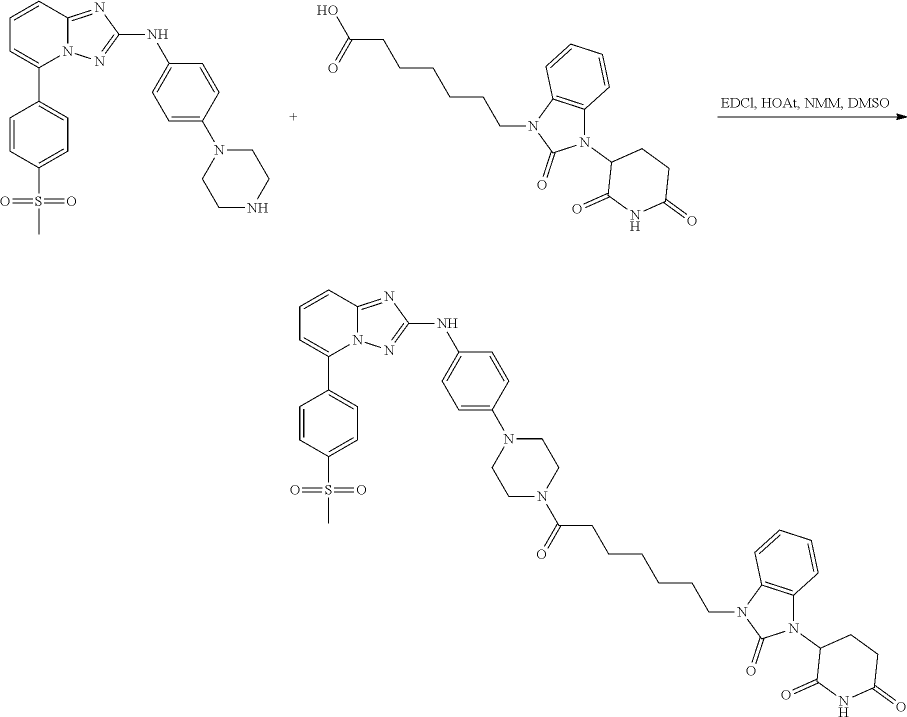 [SMILES: CS(=O)(=O)c1ccc(-c2cccc3nc(Nc4ccc(N5CCNCC5)cc4)nn23)cc1 ; O=C(O)CCCCCCn1c(=O)n(C2CCC(=O)NC2=O)c2ccccc21 ; CS(=O)(=O)c1ccc(-c2cccc3nc(Nc4ccc(N5CCN(C(=O)CCCCCCn6c(=O)n(C7CCC(=O)NC7=O)c7ccccc76)CC5)cc4)nn23)cc1]

JA-281 was synthesized following the standard procedure for preparing JA-278 (13 mg, 64% yield). MS (ESI) m/z: 805.1 [M+H]⁺.

### Example 370. 3-(4-((6-(4-(4-(8-(3,5-Difluoro-4-(morpholinomethyl)phenyl)quinoxalin-2-yl)-1H-pyrazol-1-yl)piperidin-1-yl)-6-oxohexyl)amino)-3-methyl-2-oxo-2,3-dihydro-1H-benzo[d]imidazol-1-yl)piperidine-2,6-dione (JA-282)

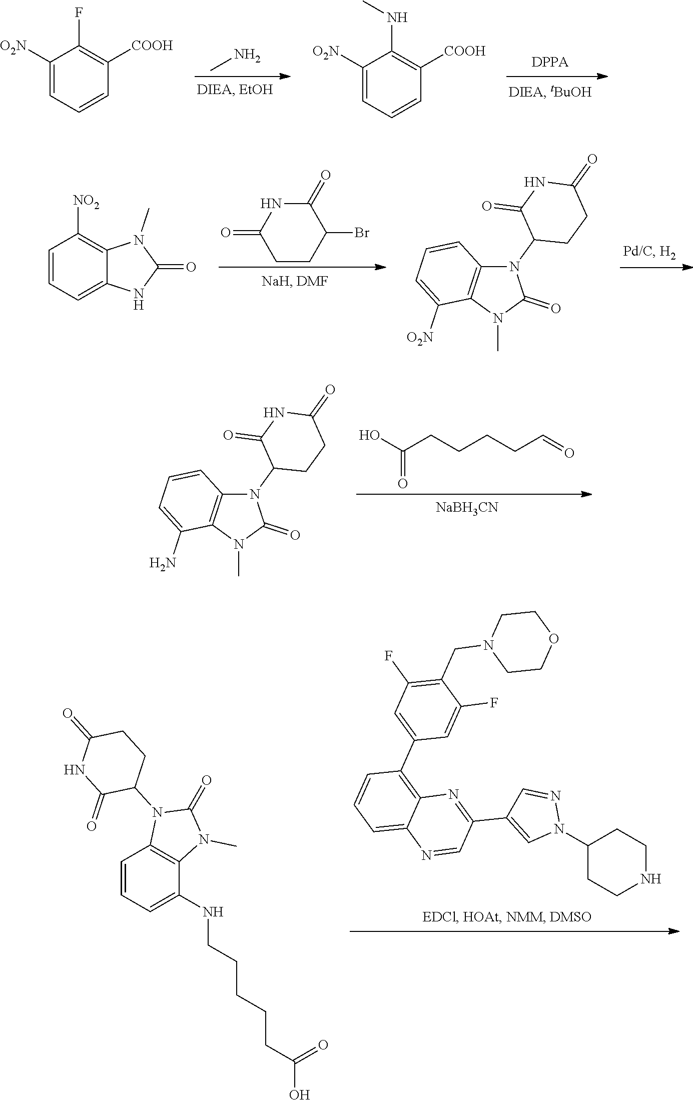

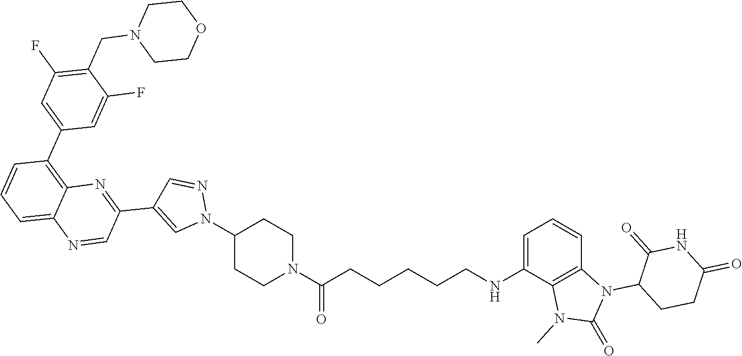 [SMILES: Cn1c(=O)n(C2CCC(=O)NC2=O)c2cccc(NCCCCCC(=O)N3CCC(n4cc(-c5cnc6cccc(-c7cc(F)c(CN8CCOCC8)c(F)c7)c6n5)cn4)CC3)c21]

### Step 1: Synthesis of 2-(methylamino)-3-nitrobenzoic acid

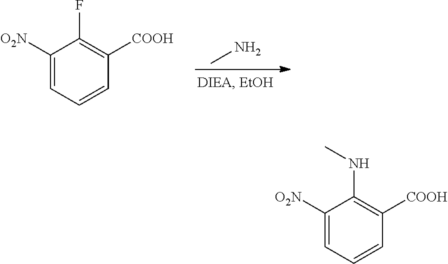

A mixture of 2-fluoro-3-nitrobenzoic acid (20 g, 108 mmol), methylamine hydrochloride (36.47 g, 540 mmol) and DIEA (167.25 g, 1.30 mol) in EtOH (300 mL) was stirred at 80° C. for 2 h. After the reaction was concentrated, the residue was poured into ice water, and the pH was adjusted to -3 with aq. HCl. The aqueous phase was extracted with EtOAc. And the combined organic layers were washed with brine, dried over Na₂SO₄, filtered and concentrated to give the desired product (24 g, 99% yield) as a yellow solid which was used directly in the next step.

### Step 2: Synthesis of 1-methyl-7-nitro-1,3-dihydro-2H-benzo[d]imidazol-2-one

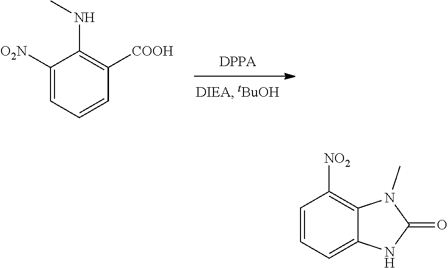

A solution of 2-(methylamino)-3-nitrobenzoic acid (24 g, 122.35 mmol), DPPA (35.71 g, 146.82 mmol) and DIEA (31.63 g, 244.70 mmol) in ^{t}BuOH (250 mL) was stirred at 90° C. overnight. After concentration, the residue was poured into ice water. The precipitate was collected by filtration, washed with water and EtOAc, dried under vacuum to give the desired product (22 g, yield 93%) as a yellow solid. (ESI) m/z: 194.1 [M+H]⁺.

### Step 3: Synthesis of 3-(3-methyl-4-nitro-2-oxo-2,3-dihydro-1H-benzo[d]imidazol-1-yl)piperidine-2,6-dione

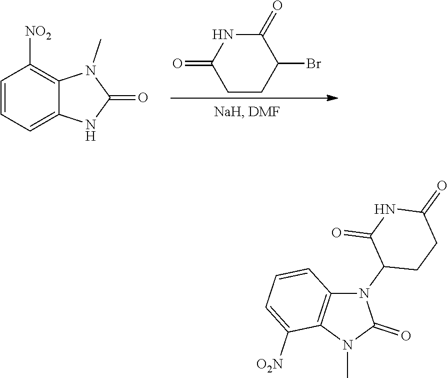

To a suspension of NaH (48 mg, 1.20 mmol) in DMF (5 mL) was added 3-methyl-5-nitro-1H-benzimidazol-2-one (193 mg, 999.18 umol) at 0° C. The reaction mixture was stirred at 0° C. for 0.5 h, before a solution of 3-bromopiperidine-2,6-dione (383.70 mg, 2.00 mmol) in DMF (5 mL) was added dropwise. After the completion of addition, the reaction mixture was stirred at 80° C. for 2 h. After concentration, the resulting residue was purified by prep-HPLC to give the desired product (80 mg, yield 26%) as a black solid. MS (ESI) m/z: 305.3 [M+H]⁺.

### Step 4: Synthesis of 3-(4-amino-3-methyl-2-oxo-2,3-dihydro-1H-benzo[d]imidazol-1-yl)piperidine-2,6-dione

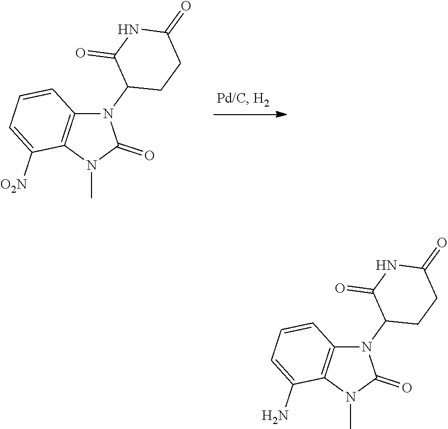 [SMILES: Cn1c(=O)n(C2CCC(=O)NC2=O)c2cccc([N+](=O)[O-])c21 ; Cn1c(=O)n(C2CCC(=O)NC2=O)c2cccc(N)c21]

To a solution of 3-(3-methyl-4-nitro-2-oxo-benzimidazol-1-yl)piperidine-2,6-dione (80 mg, 262.93 umol) in MeOH (10 mL) and THF (10 mL) was added Pd/C (20 mg) at room temperature. After the reaction mixture was stirred at room temperature for 1 h under hydrogen atmosphere, the reaction was filtered and concentrated to give the desired product (70 mg, yield 97%) as a brown solid, which was used in the next step directly without further purification. MS (ESI) m/z: 275.3 [M+H]⁺.

### Step 5: Synthesis of 6-((1-(2,6-dioxopiperidin-3-yl)-3-methyl-2-oxo-2,3-dihydro-1H-benzo[d]imidazol-4-yl)amino)hexanoic acid

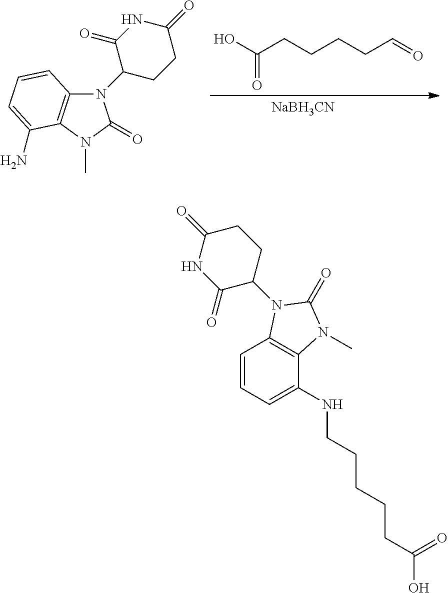

A mixture of 3-(4-amino-3-methyl-2-oxo-2,3-dihydro-1H-benzo[d]imidazol-1-yl)piperidine-2,6-dione (5 mg, 0.018 mmol) and 6-oxohexanoic acid (4.74 mg, 0.036 mmol) in ⁱPrOH (2 mL) and AcOH (2 mL) was stirred at 90° C. for 4 h. After the reaction was cooled to room temperature, NaBH₃CN (2.3 mg, 0.036 mmol) was added. The reaction mixture was stirred at room temperature for another 2 h, before it was purified by prep-TLC (DCM/MeOH=10:1) to give the desired product (3 mg, yield 42%) as a white solid. MS (ESI) m/z: 389.7 [M+H]⁺.

### Step 6: Synthesis of 3-(4-((6-(4-(4-(8-(3,5-difluoro-4-(morpholinomethyl)phenyl)quinoxalin-2-yl)-1H-pyrazol-1-yl)piperidin-1-yl)-6-oxohexyl)amino)-3-methyl-2-oxo-2,3-dihydro-1H-benzo[d]imidazol-1-yl)piperidine-2,6-dione (JA-282)

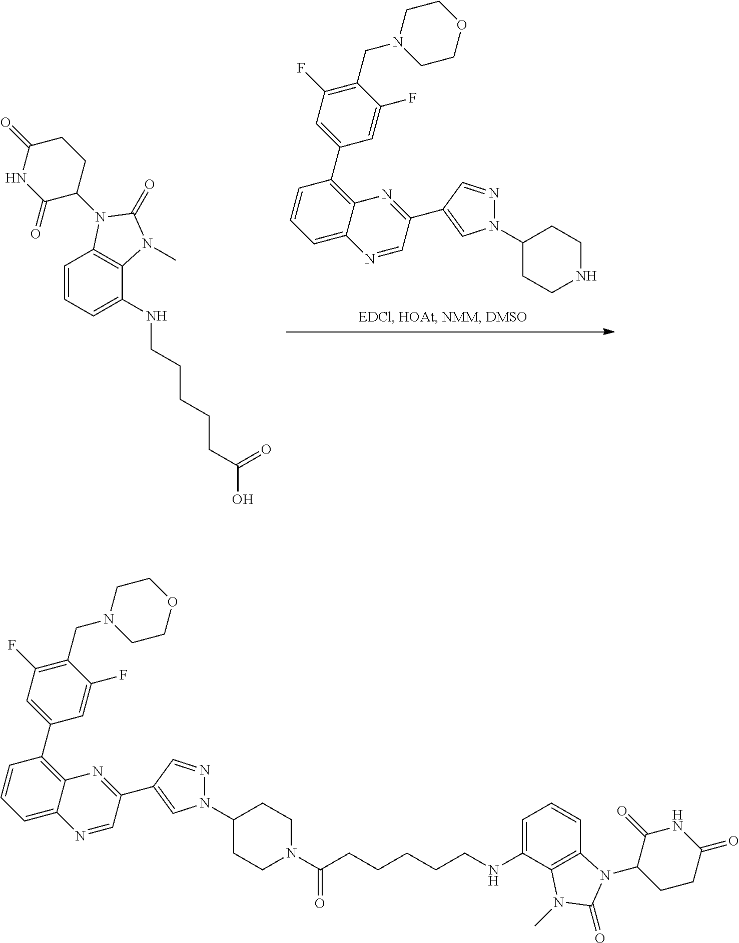 [SMILES: Cn1c(=O)n(C2CCC(=O)NC2=O)c2cccc(NCCCCCC(=O)O)c21 ; Fc1cc(-c2cccc3ncc(-c4cnn(C5CCNCC5)c4)nc23)cc(F)c1CN1CCOCC1 ; Cn1c(=O)n(C2CCC(=O)NC2=O)c2cccc(NCCCCCC(=O)N3CCC(n4cc(-c5cnc6cccc(-c7cc(F)c(CN8CCOCC8)c(F)c7)c6n5)cn4)CC3)c21]

JA-282 was synthesized following the standard procedure for preparing JA-001 (2.0 mg, 23% yield). MS (ESI) m/z: 862.1 [M+H]⁺.

### Example 371. 3-(4-((7-(4-(4-(8-(3,5-Difluoro-4-(morpholinomethyl)phenyl)quinoxalin-2-yl)-1H-pyrazol-1-yl)piperidin-1-yl)-7-oxoheptyl)amino)-3-methyl-2-oxo-2,3-dihydro-1H-benzo[d]imidazol-1-yl)piperidine-2,6-dione (JA-283)

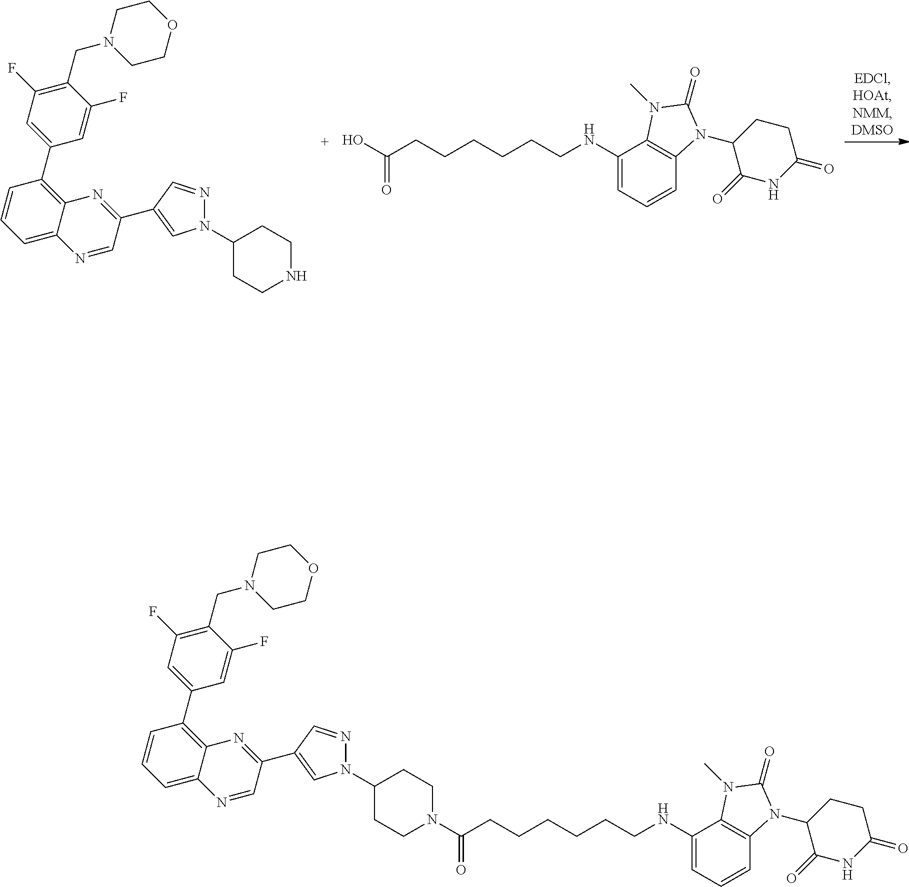 [SMILES: Fc1cc(-c2cccc3ncc(-c4cnn(C5CCNCC5)c4)nc23)cc(F)c1CN1CCOCC1 ; Cn1c(=O)n(C2CCC(=O)NC2=O)c2cccc(NCCCCCCC(=O)O)c21 ; Cn1c(=O)n(C2CCC(=O)NC2=O)c2cccc(NCCCCCCC(=O)N3CCC(n4cc(-c5cnc6cccc(-c7cc(F)c(CN8CCOCC8)c(F)c7)c6n5)cn4)CC3)c21]

JA-283 was synthesized following the standard procedure for preparing JA-282 (2.0 mg, 15% yield). MS (ESI) m/z: 876.2 [M+H]⁺.

### Example 372. 3-(3-Methyl-4-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)-2-oxo-2,3-dihydro-1H-benzo[d]imidazol-1-yl)piperidine-2,6-dione (JA-284)

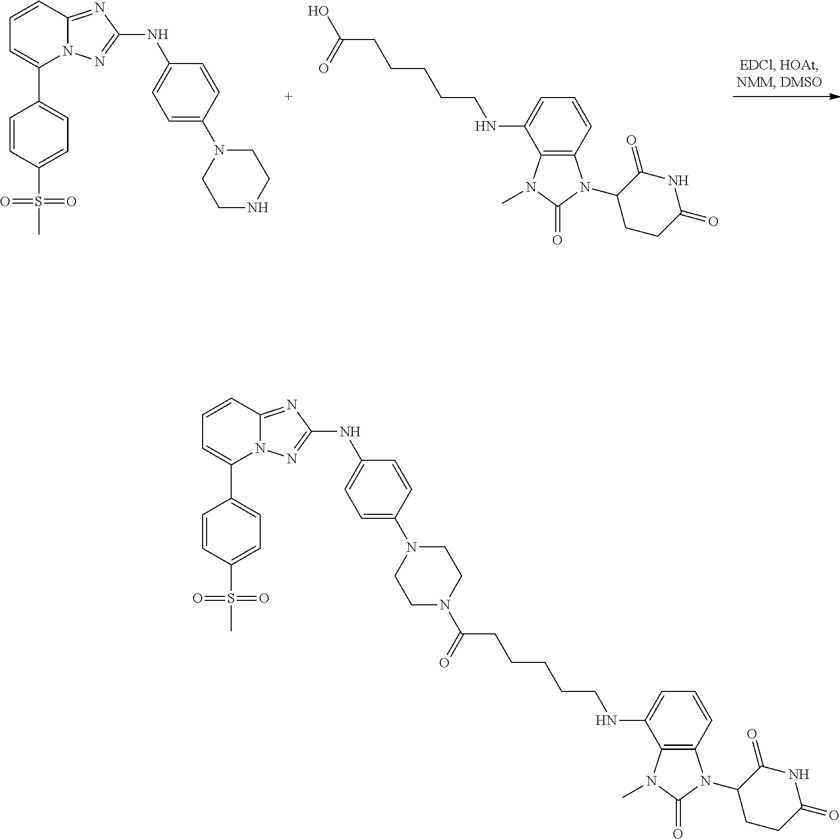 [SMILES: CS(=O)(=O)c1ccc(-c2cccc3nc(Nc4ccc(N5CCNCC5)cc4)nn23)cc1 ; Cn1c(=O)n(C2CCC(=O)NC2=O)c2cccc(NCCCCCC(=O)O)c21 ; Cn1c(=O)n(C2CCC(=O)NC2=O)c2cccc(NCCCCCC(=O)N3CCN(c4ccc(Nc5nc6cccc(-c7ccc(S(C)(=O)=O)cc7)n6n5)cc4)CC3)c21]

JA-284 was synthesized following the standard procedure for preparing JA-282 (2.0 mg, 24% yield). MS (ESI) m/z: 820.0 [M+H]⁺.

### Example 373. 3-(3-Methyl-4-((7-(4-(4-((5-(4-(methylsulfonyl)phenyl)-[1,2,4]triazolo[1,5-a]pyridin-2-yl)amino)phenyl)piperazin-1-yl)-7-oxoheptyl)amino)-2-oxo-2,3-dihydro-1H-benzo[d]imidazol-1-yl)piperidine-2,6-dione (JA-285)

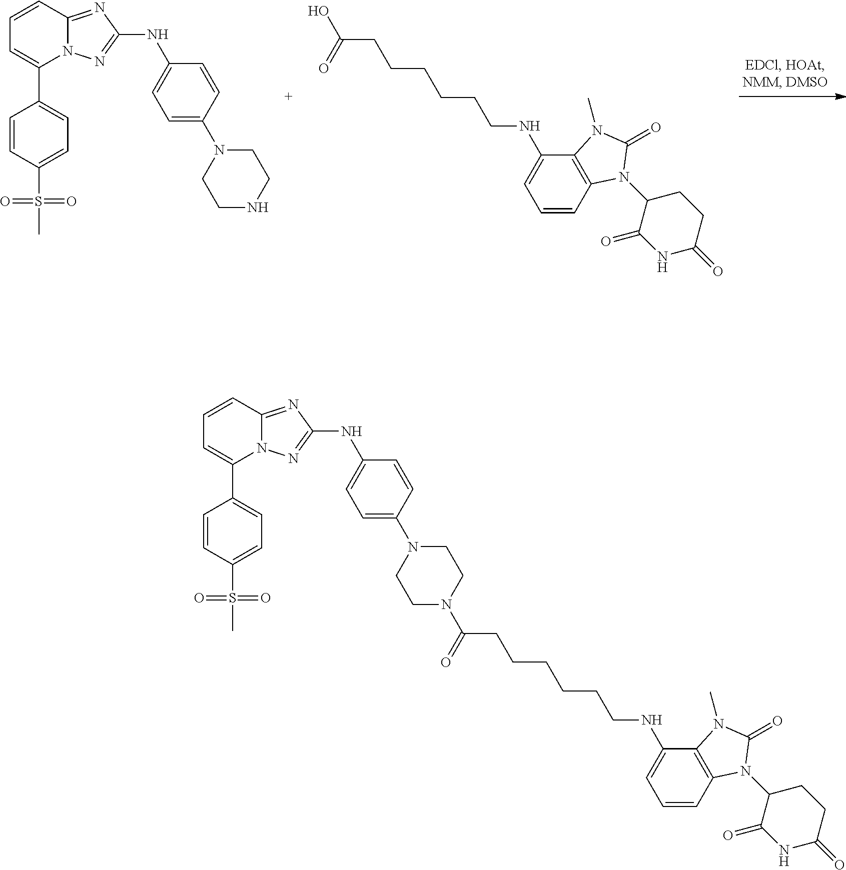 [SMILES: CS(=O)(=O)c1ccc(-c2cccc3nc(Nc4ccc(N5CCNCC5)cc4)nn23)cc1 ; Cn1c(=O)n(C2CCC(=O)NC2=O)c2cccc(NCCCCCCC(=O)O)c21 ; Cn1c(=O)n(C2CCC(=O)NC2=O)c2cccc(NCCCCCCC(=O)N3CCN(c4ccc(Nc5nc6cccc(-c7ccc(S(C)(=O)=O)cc7)n6n5)cc4)CC3)c21]

JA-285 was synthesized following the standard procedure for preparing JA-282 (2.0 mg, 16% yield). MS (ESI) m/z: 834.1 [M+H]⁺.

### Example 374. 3-(4-((5-(4-(4-(8-(3,5-Difluoro-4-(morpholinomethyl)phenyl)quinoxalin-2-yl)-1H-pyrazol-1-yl)piperidin-1-yl)-5-oxopentyl)amino)-3-methyl-2-oxo-2,3-dihydro-1H-benzo[d]imidazol-1-yl)piperidine-2,6-dione (JA-286)

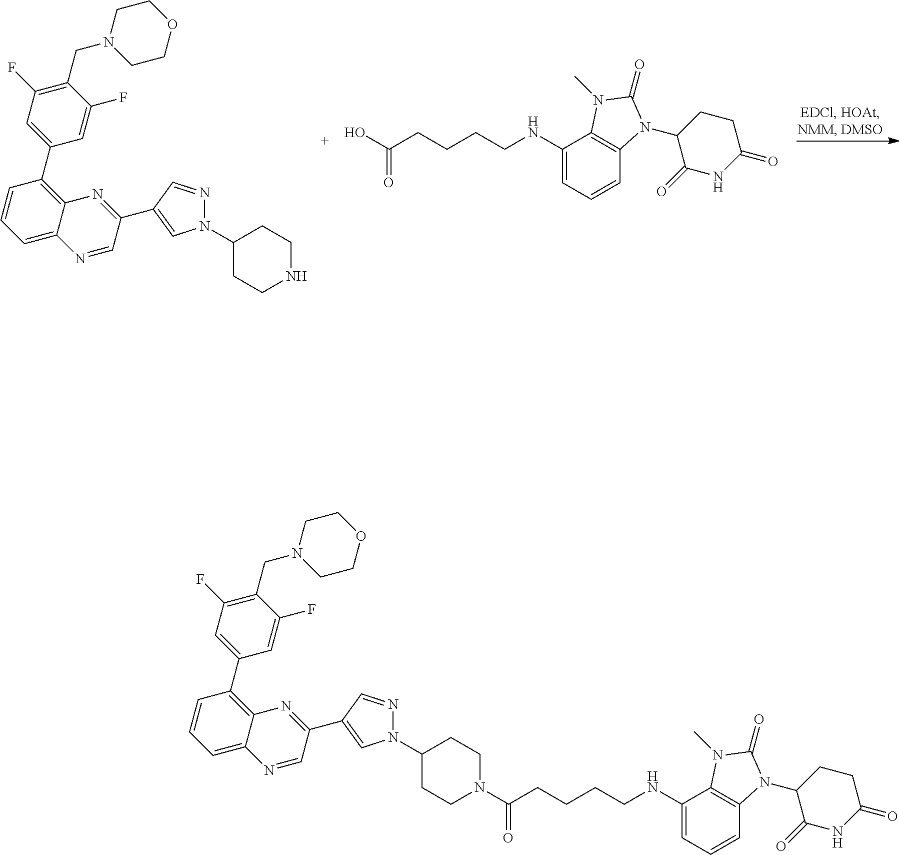 [SMILES: Fc1cc(-c2cccc3ncc(-c4cnn(C5CCNCC5)c4)nc23)cc(F)c1CN1CCOCC1 ; Cn1c(=O)n(C2CCC(=O)NC2=O)c2cccc(NCCCCC(=O)O)c21 ; Cn1c(=O)n(C2CCC(=O)NC2=O)c2cccc(NCCCCC(=O)N3CCC(n4cc(-c5cnc6cccc(-c7cc(F)c(CN8CCOCC8)c(F)c7)c6n5)cn4)CC3)c21]

JA-286 was synthesized following the standard procedure for preparing JA-282 (4.3 mg, 19% yield). MS (ESI) m/z: 848.0 [M+H]⁺.

### Example 375. 3-(5-((5-(4-(4-(8-(3,5-Difluoro-4-(morpholinomethyl)phenyl)quinoxalin-2-yl)-1H-pyrazol-1-yl)piperidin-1-yl)-5-oxopentyl)amino)-3-methyl-2-oxo-2,3-dihydro-1H-benzo[d]imidazol-1-yl)piperidine-2,6-dione (JA-287)

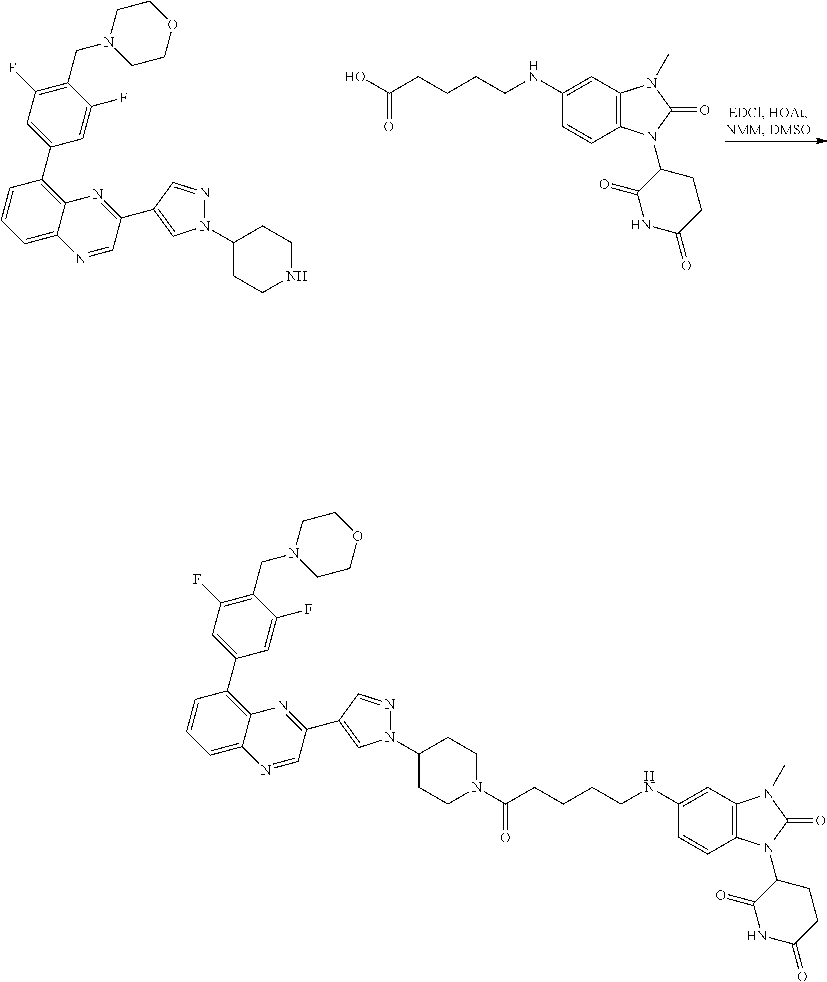 [SMILES: Fc1cc(-c2cccc3ncc(-c4cnn(C5CCNCC5)c4)nc23)cc(F)c1CN1CCOCC1 ; Cn1c(=O)n(C2CCC(=O)NC2=O)c2ccc(NCCCCC(=O)O)cc21 ; Cn1c(=O)n(C2CCC(=O)NC2=O)c2ccc(NCCCCC(=O)N3CCC(n4cc(-c5cnc6cccc(-c7cc(F)c(CN8CCOCC8)c(F)c7)c6n5)cn4)CC3)cc21]

JA-287 was synthesized following the standard procedure for preparing JA-282 (2.4 mg, 11% yield). MS (ESI) m/z: 848.0 [M+H]⁺.

### Example 376. 3-(5-((6-(4-(4-(8-(3,5-Difluoro-4-(morpholinomethyl)phenyl)quinoxalin-2-yl)-1H-pyrazol-1-yl)piperidin-1-yl)-6-oxohexyl)amino)-3-methyl-2-oxo-2,3-dihydro-1H-benzo[d]imidazol-1-yl)piperidine-2,6-dione (JA-288)

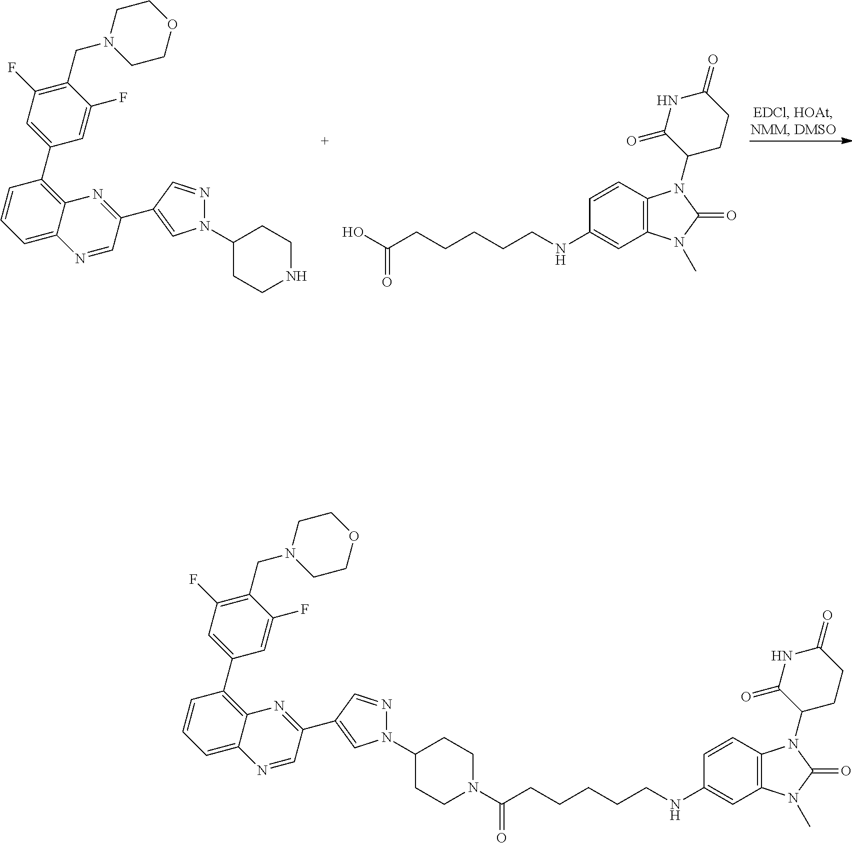 [SMILES: Fc1cc(-c2cccc3ncc(-c4cnn(C5CCNCC5)c4)nc23)cc(F)c1CN1CCOCC1 ; Cn1c(=O)n(C2CCC(=O)NC2=O)c2ccc(NCCCCCC(=O)O)cc21 ; Cn1c(=O)n(C2CCC(=O)NC2=O)c2ccc(NCCCCCC(=O)N3CCC(n4cc(-c5cnc6cccc(-c7cc(F)c(CN8CCOCC8)c(F)c7)c6n5)cn4)CC3)cc21]

JA-288 was synthesized following the standard procedure for preparing JA-282 (5.1 mg, 26% yield). MS (ESI) m/z: 861.9 [M+H]⁺.

### Example 377. 3-(5-((7-(4-(4-(8-(3,5-Difluoro-4-(morpholinomethyl)phenyl)quinoxalin-2-yl)-1H-pyrazol-1-yl)piperidin-1-yl)-7-oxoheptyl)amino)-3-methyl-2-oxo-2,3-dihydro-1H-benzo[d]imidazol-1-yl)piperidine-2,6-dione (JA-289)

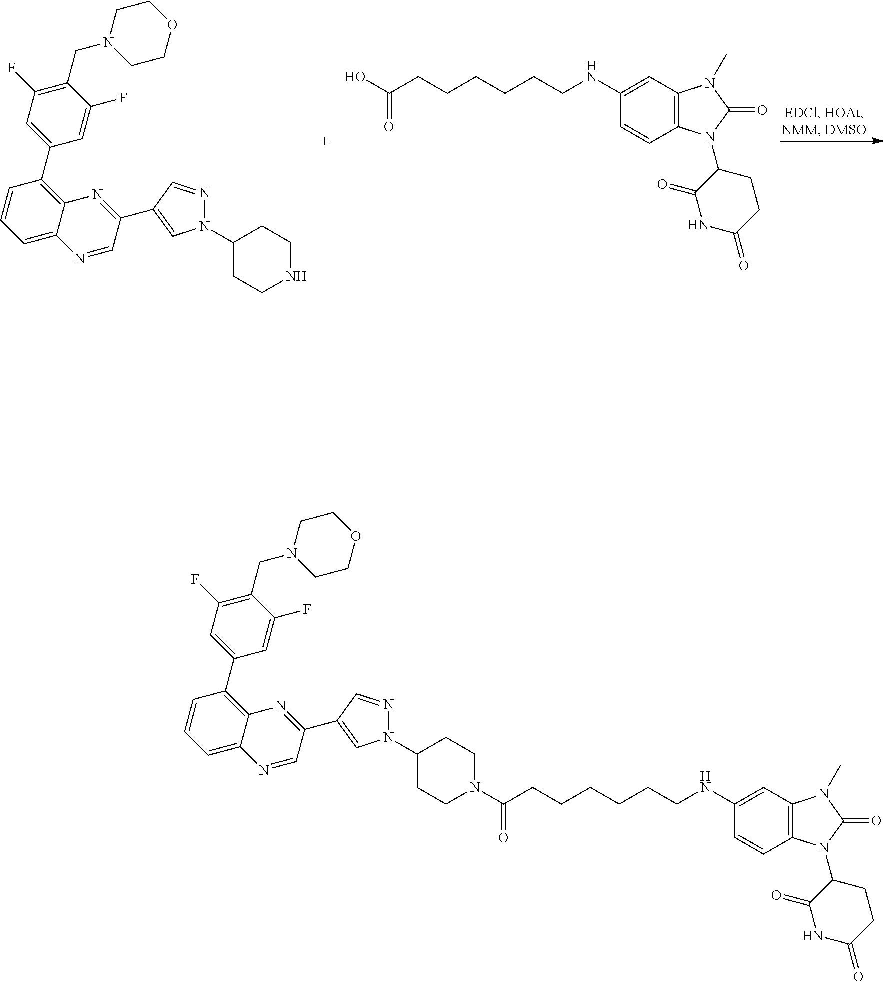 [SMILES: Fc1cc(-c2cccc3ncc(-c4cnn(C5CCNCC5)c4)nc23)cc(F)c1CN1CCOCC1 ; Cn1c(=O)n(C2CCC(=O)NC2=O)c2ccc(NCCCCCCC(=O)O)cc21 ; Cn1c(=O)n(C2CCC(=O)NC2=O)c2ccc(NCCCCCCC(=O)N3CCC(n4cc(-c5cnc6cccc(-c7cc(F)c(CN8CCOCC8)c(F)c7)c6n5)cn4)CC3)cc21]

JA-289 was synthesized following the standard procedure for preparing JA-282 (3.9 mg, 36% yield). MS (ESI) m/z: 876.1 [M+H]⁺.

### Example 378. 3-(3-Methyl-4-((5-(4-(4-((5-(4-(methylsulfonyl)phenyl)-[1,2,4]triazolo[1,5-a]pyridin-2-yl)amino)phenyl)piperazin-1-yl)-5-oxopentyl)amino)-2-oxo-2,3-dihydro-1H-benzo[d]imidazol-1-yl)piperidine-2,6-dione (JA-290)

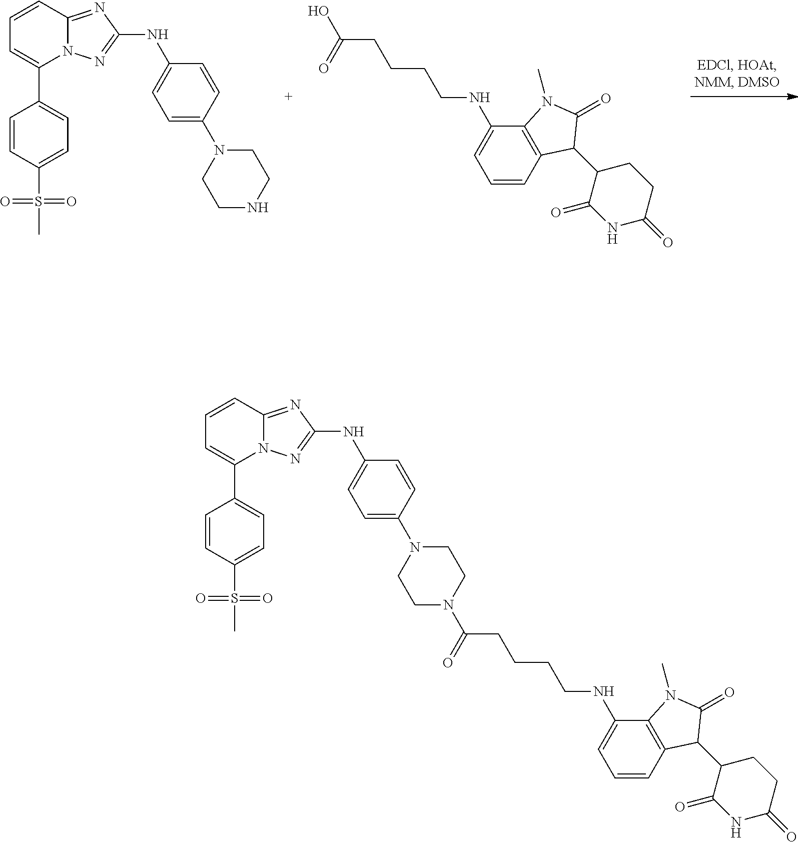 [SMILES: CS(=O)(=O)c1ccc(-c2cccc3nc(Nc4ccc(N5CCNCC5)cc4)nn23)cc1 ; CN1C(=O)C(C2CCC(=O)NC2=O)c2cccc(NCCCCC(=O)O)c21 ; CN1C(=O)C(C2CCC(=O)NC2=O)c2cccc(NCCCCC(=O)N3CCN(c4ccc(Nc5nc6cccc(-c7ccc(S(C)(=O)=O)cc7)n6n5)cc4)CC3)c21]

JA-290 was synthesized following the standard procedure for preparing JA-282 (2.1 mg, 10% yield). MS (ESI) m/z: 805.9 [M+H]⁺.

### Example 379. 3-(3-Methyl-5-((5-(4-(4-((5-(4-(methylsulfonyl)phenyl)-[1,2,4]triazolo[1,5-a]pyridin-2-yl)amino)phenyl)piperazin-1-yl)-5-oxopentyl)amino)-2-oxo-2,3-dihydro-1H-benzo[d]imidazol-1-yl)piperidine-2,6-dione (JA-291)

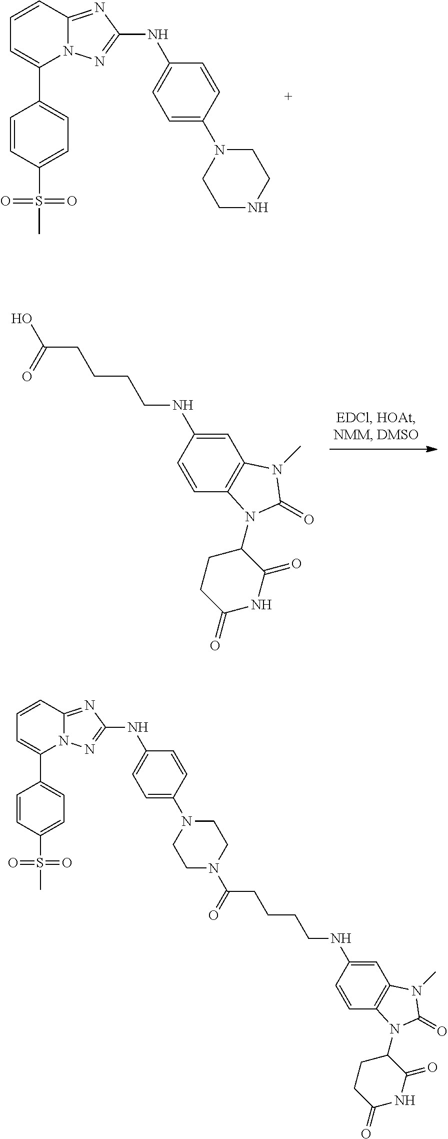 [SMILES: CS(=O)(=O)c1ccc(-c2cccc3nc(Nc4ccc(N5CCNCC5)cc4)nn23)cc1 ; Cn1c(=O)n(C2CCC(=O)NC2=O)c2ccc(NCCCCC(=O)O)cc21 ; Cn1c(=O)n(C2CCC(=O)NC2=O)c2ccc(NCCCCC(=O)N3CCN(c4ccc(Nc5nc6cccc(-c7ccc(S(C)(=O)=O)cc7)n6n5)cc4)CC3)cc21]

JA-291 was synthesized following the standard procedure for preparing JA-282 (1.8 mg, 8% yield). MS (ESI) m/z: 805.8 [M+H]⁺.

### Example 380. 3-(3-Methyl-5-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)-2-oxo-2,3-dihydro-1H-benzo[d]imidazol-1-yl)piperidine-2,6-dione (JA-292)

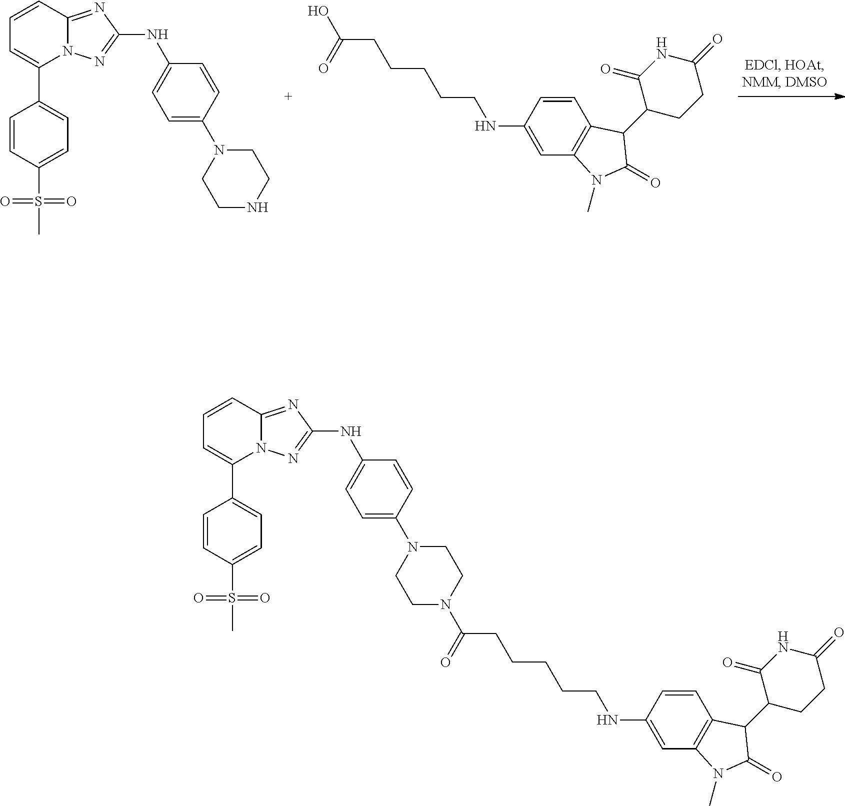 [SMILES: CS(=O)(=O)c1ccc(-c2cccc3nc(Nc4ccc(N5CCNCC5)cc4)nn23)cc1 ; CN1C(=O)C(C2CCC(=O)NC2=O)c2ccc(NCCCCCC(=O)O)cc21 ; CN1C(=O)C(C2CCC(=O)NC2=O)c2ccc(NCCCCCC(=O)N3CCN(c4ccc(Nc5nc6cccc(-c7ccc(S(C)(=O)=O)cc7)n6n5)cc4)CC3)cc21]

JA-292 was synthesized following the standard procedure for preparing JA-282 (4.7 mg, 25% yield). MS (ESI) m/z: 819.9 [M+H]⁺.

### Example 381. 3-(3-Methyl-5-((7-(4-(4-((5-(4-(methylsulfonyl)phenyl)-[1,2,4]triazolo[1,5-a]pyridin-2-yl)amino)phenyl)piperazin-1-yl)-7-oxoheptyl)amino)-2-oxo-2,3-dihydro-1H-benzo[d]imidazol-1-yl)piperidine-2,6-dione (JA-293)

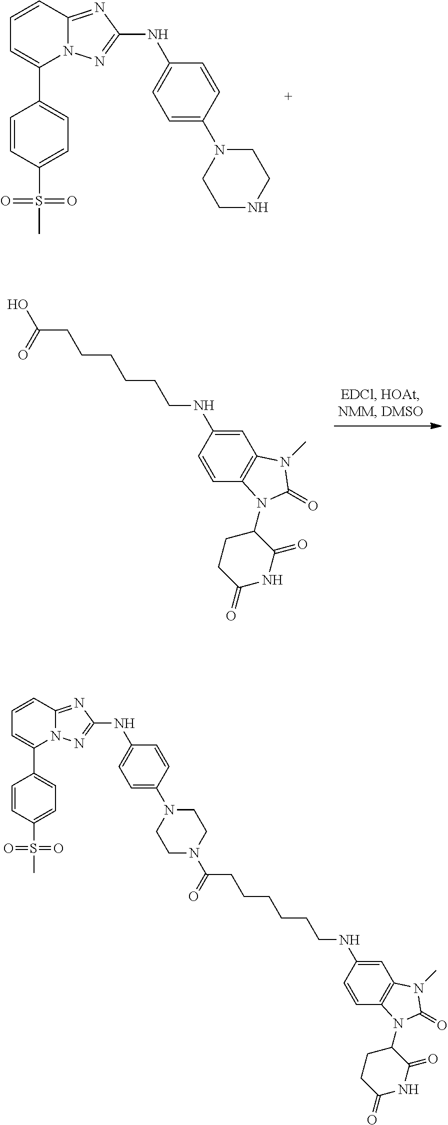 [SMILES: CS(=O)(=O)c1ccc(-c2cccc3nc(Nc4ccc(N5CCNCC5)cc4)nn23)cc1 ; Cn1c(=O)n(C2CCC(=O)NC2=O)c2ccc(NCCCCCCC(=O)O)cc21 ; Cn1c(=O)n(C2CCC(=O)NC2=O)c2ccc(NCCCCCCC(=O)N3CCN(c4ccc(Nc5nc6cccc(-c7ccc(S(C)(=O)=O)cc7)n6n5)cc4)CC3)cc21]

JA-293 was synthesized following the standard procedure for preparing JA-282 (4.6 mg, 44% yield). MS (ESI) m/z: 833.9 [M+H]⁺.

Certain compounds disclosed herein have the structures shown in Table 1.

TABLE 1

| Cpd. | | |
|---|---|---|
| Code | Structure | Chemical Name |
| JA-001 | 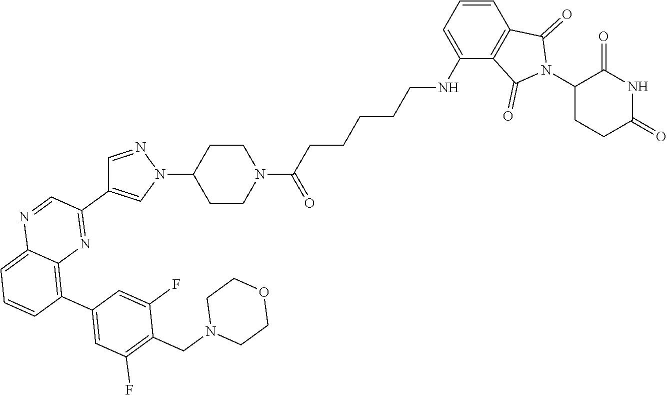 [SMILES: O=C1CCC(N2C(=O)c3cccc(NCCCCCC(=O)N4CCC(n5cc(-c6cnc7cccc(-c8cc(F)c(CN9CCOCC9)c(F)c8)c7n6)cn5)CC4)c3C2=O)C(=O)N1] | 4-[[6-[4-[4-[8-[3,5-difluoro- 4- (morpholinomethyl)phenyl] quinoxalin-2-yl]pyrazol-1-yl]- 1-piperidyl]-6-oxo- hexyl]amino]-2-(2,6-dioxo-3- piperidyl)isoindoline-1,3- dione |
| JA-002 | 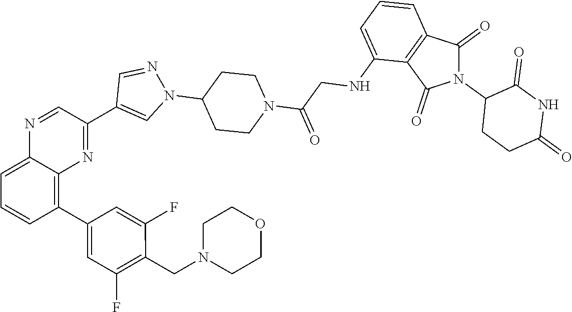 [SMILES: O=C1CCC(N2C(=O)c3cccc(NCC(=O)N4CCC(n5cc(-c6cnc7cccc(-c8cc(F)c(CN9CCOCC9)c(F)c8)c7n6)cn5)CC4)c3C2=O)C(=O)N1] | 4-[[2-[4-[4-[8-[3,5-difluoro- 4- (morpholinomethyl)phenyl] quinoxalin-2-yl]pyrazol-1-yl]- 1-piperidyl]-2-oxo- ethyl]amino]-2-(2,6-dioxo-3- piperidyl)isoindoline-1,3- dione |
| JA-003 | 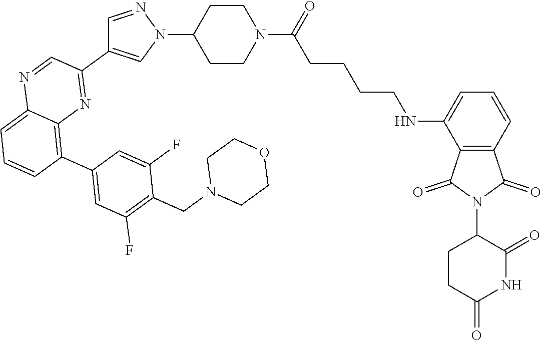 [SMILES: O=C1CCC(N2C(=O)c3cccc(NCCCCC(=O)N4CCC(n5cc(-c6cnc7cccc(-c8cc(F)c(CN9CCOCC9)c(F)c8)c7n6)cn5)CC4)c3C2=O)C(=O)N1] | 4-[[5-[4-[4-[8-[3,5-difluoro- 4- (morpholinomethyl)phenyl] quinoxalin-2-yl]pyrazol-1-yl]- 1-piperidyl]-5-oxo- pentyl]amino]-2-(2,6-dioxo- 3-piperidyl)isoindoline-1,3- dione |
| JA-004 | 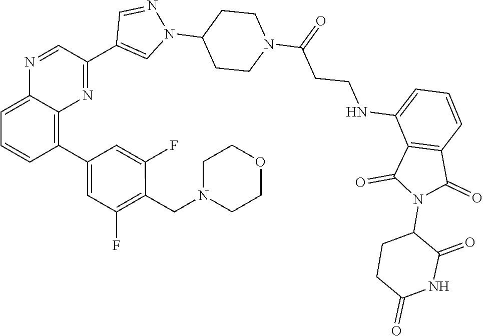 [SMILES: O=C1CCC(N2C(=O)c3cccc(NCCC(=O)N4CCC(n5cc(-c6cnc7cccc(-c8cc(F)c(CN9CCOCC9)c(F)c8)c7n6)cn5)CC4)c3C2=O)C(=O)N1] | 4-[[3-[4-[4-[8-[3,5-difluoro- 4- (morpholinomethyl)phenyl] quinoxalin-2-yl]pyrazol-1-yl]- 1-piperidyl]-3-oxo- propyl]amino]-2-(2,6-dioxo- 3-piperidyl)isoindoline-1,3- dione |
| JA-005 | 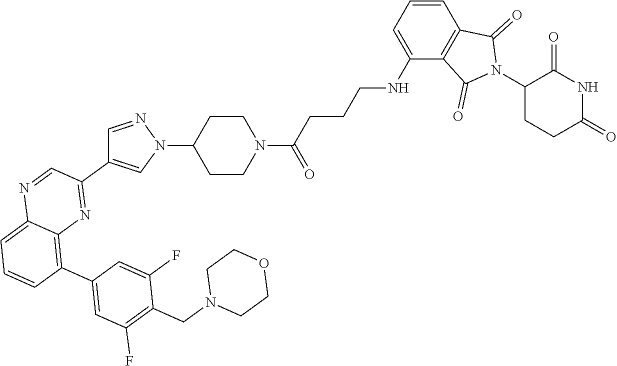 [SMILES: O=C1CCC(N2C(=O)c3cccc(NCCCC(=O)N4CCC(n5cc(-c6cnc7cccc(-c8cc(F)c(CN9CCOCC9)c(F)c8)c7n6)cn5)CC4)c3C2=O)C(=O)N1] | 4-[[4-[4-[4-[8-[3,5-difluoro- 4- (morpholinomethyl)phenyl] quinoxalin-2-yl]pyrazol-1-yl]- 1-piperidyl]-4-oxo- butyl]amino]-2-(2,6-dioxo-3- piperidyl)isoindoline-1,3- dione |
| JA-006 | 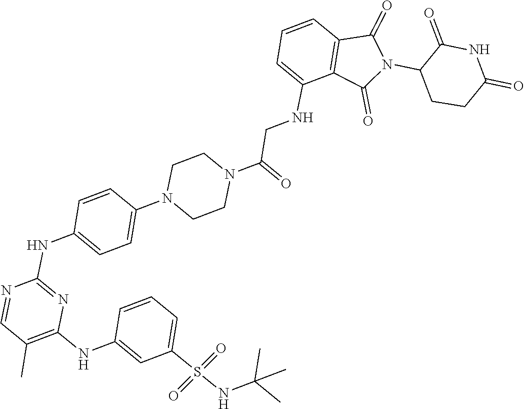 [SMILES: Cc1cnc(Nc2ccc(N3CCN(C(=O)CNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4- ((2-(2,6-dioxopiperidin-3-yl)- 1,3-dioxoisoindolin-4- yl)glycyl)piperazin-1- yl)phenyl)amino)-5- methylpyrimidin-4- yl)amino)benzenesulfonamide |
| JA-007 | 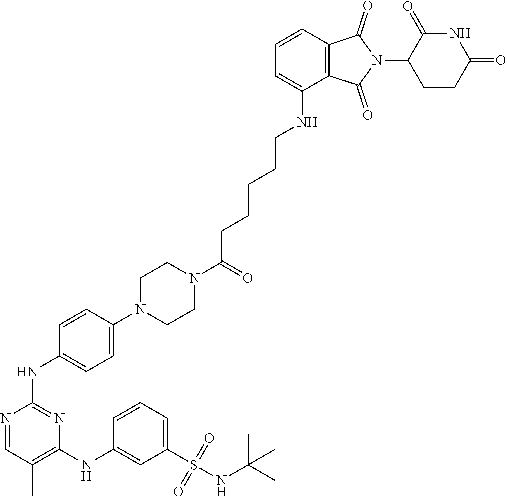 [SMILES: Cc1cnc(Nc2ccc(N3CCN(C(=O)CCCCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(6- ((2-(2,6-dioxopiperidin-3-yl)- 1,3-dioxoisoindolin-4- yl)amino)hexanoyl)piperazin- 1-yl)phenyl)amino)-5- methylpyrimidin-4- yl)amino)benzenesulfonamide |
| JA-008 | 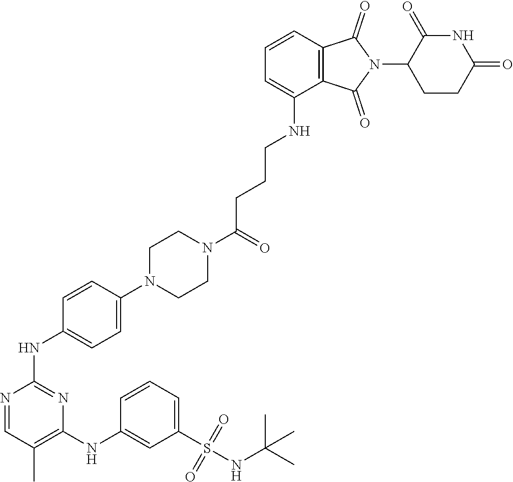 [SMILES: Cc1cnc(Nc2ccc(N3CCN(C(=O)CCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(4- ((2-(2,6-dioxopiperidin-3-yl)- 1,3-dioxoisoindolin-4- yl)amino)butanoyl)piperazin- 1-yl)phenyl)amino)-5- methylpyrimidin-4- yl)amino)benzenesulfonamide |
| JA-009 | 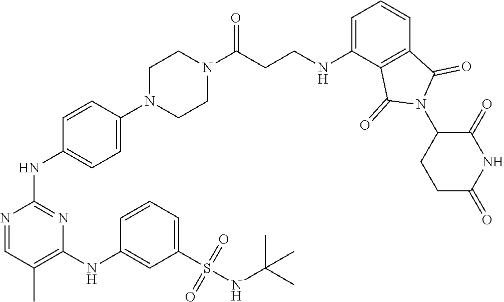 [SMILES: Cc1cnc(Nc2ccc(N3CCN(C(=O)CCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- ((2-(2,6-dioxopiperidin-3-yl)- 1,3-dioxoisoindolin-4- yl)amino)propanoyl)piperazin- 1-yl)phenyl)amino)-5- methylpyrimidin-4- yl)amino)benzenesulfonamide |
| JA-010 | 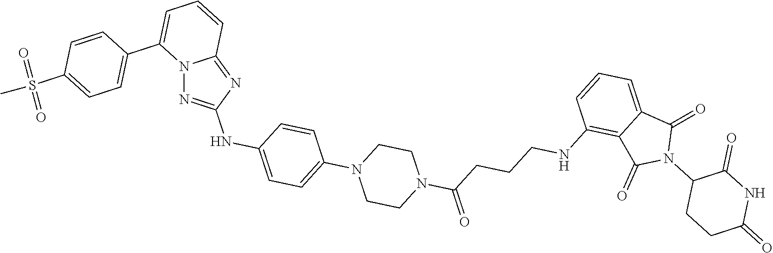 [SMILES: CS(=O)(=O)c1ccc(-c2cccc3nc(Nc4ccc(N5CCN(C(=O)CCCNc6cccc7c6C(=O)N(C6CCC(=O)NC6=O)C7=O)CC5)cc4)nn23)cc1] | 2-(2,6-dioxopiperidin-3-yl)- 4-((4-(4-(4-((5-(4- (methylsulfonyl)phenyl)- [1,2,4]triazolo[1,5-a]pyridin- 2-yl)amino)phenyl)piperazin- 1-yl)-4- oxobutyl)amino)isoindoline- 1,3-dione |
| JA-011 | 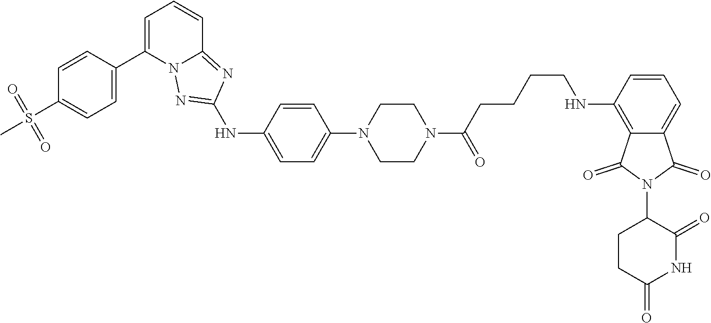 [SMILES: CS(=O)(=O)c1ccc(-c2cccc3nc(Nc4ccc(N5CCN(C(=O)CCCCNc6cccc7c6C(=O)N(C6CCC(=O)NC6=O)C7=O)CC5)cc4)nn23)cc1] | 2-(2,6-Dioxo-piperidin-3-yl)- 4-[5-(4-{4-[5-(4- methanesulfonyl-phenyl)- [1,2,4]triazolo[1,5-a]pyridin- 2-ylamino]-phenyl}- piperazin-1-yl)-5-oxo- pentylamino]-isoindole-1,3- dione |
| JA-012 | 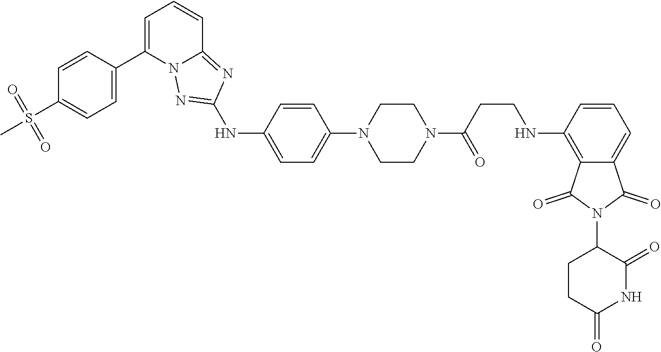 [SMILES: CS(=O)(=O)c1ccc(-c2cccc3nc(Nc4ccc(N5CCN(C(=O)CCNc6cccc7c6C(=O)N(C6CCC(=O)NC6=O)C7=O)CC5)cc4)nn23)cc1] | 2-(2,6-Dioxo-piperidin-3-yl)- 4-[3-(4-{4-[5-(4- methanesulfonyl-phenyl)- [1,2,4]triazolo[1,5-a]pyridin- 2-ylamino]-phenyl}- piperazin-1-yl)-3-oxo- propylamino]-isoindole-1,3- dione |
| JA-013 | 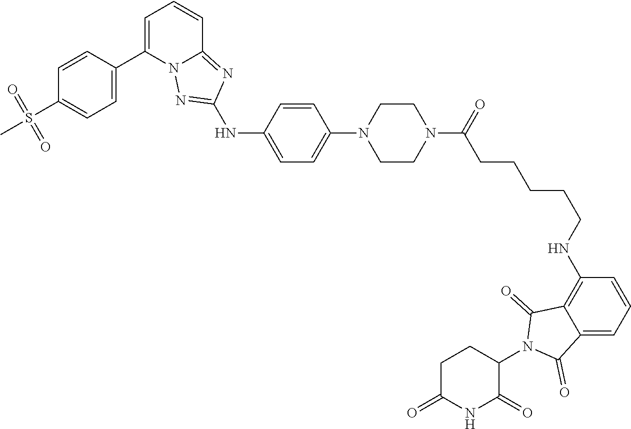 [SMILES: CS(=O)(=O)c1ccc(-c2cccc3nc(Nc4ccc(N5CCN(C(=O)CCCCCNc6cccc7c6C(=O)N(C6CCC(=O)NC6=O)C7=O)CC5)cc4)nn23)cc1] | 2-(2,6-Dioxo-piperidin-3-yl)- 4-[6-(4-{4-[5-(4- methanesulfonyl-phenyl)- [1,2,4]triazolo[1,5-a]pyridin- 2-ylamino]-phenyl}- piperazin-1-yl)-6-oxo- hexylamino]-isoindole-1,3- dione |
| JA-014 | 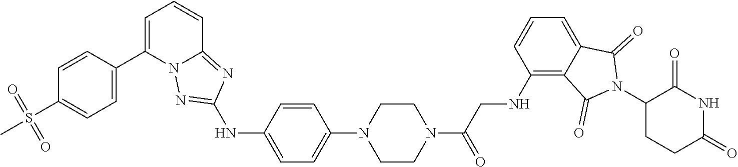 [SMILES: CS(=O)(=O)c1ccc(-c2cccc3nc(Nc4ccc(N5CCN(C(=O)CNc6cccc7c6C(=O)N(C6CCC(=O)NC6=O)C7=O)CC5)cc4)nn23)cc1] | 2-(2,6-Dioxo-piperidin-3-yl)- 4-[2-(4-{4-[5-(4- methanesulfonyl-phenyl)- [1,2,4]triazolo[1,5-a]pyridin- 2-ylamino]-phenyl}- piperazin-1-yl)-2-oxo- ethylamino]-isoindole-1,3- dione |
| JA-015 | 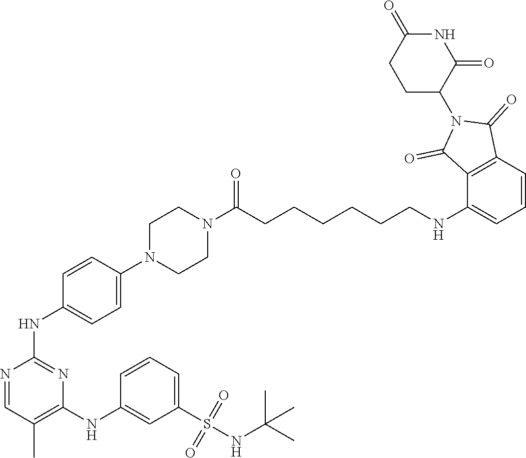 [SMILES: Cc1cnc(Nc2ccc(N3CCN(C(=O)CCCCCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(7- ((2-(2,6-dioxopiperidin-3-yl)- 1,3-dioxoisoindolin-4- yl)amino)heptanoyl)piperazin- yl)phenyl)amino)-5- methylpyrimidin-4- yl)amino)benzenesulfonamide |
| JA-016 | 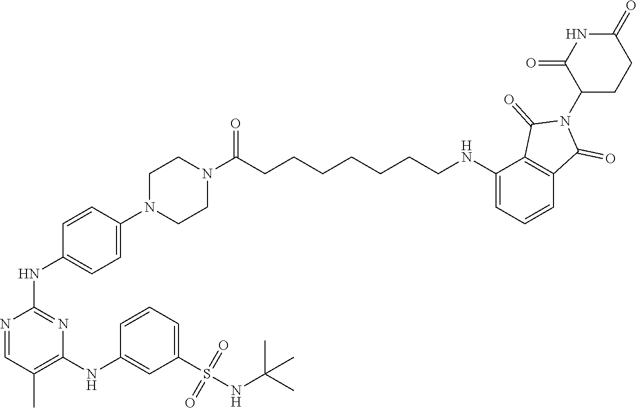 [SMILES: Cc1cnc(Nc2ccc(N3CCN(C(=O)CCCCCCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(8- ((2-(2,6-dioxopiperidin-3-yl)- 1,3-dioxoisoindolin-4- yl)amino)octanoyl)piperazin- 1-yl)phenyl)amino)-5- methylpyrimidin-4- yl)amino)benzenesulfonamide |
| JA-017 | 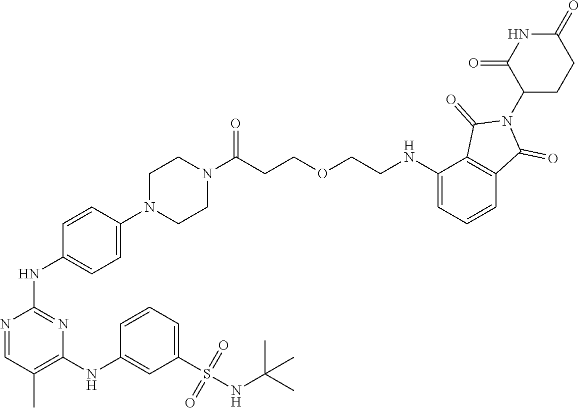 [SMILES: Cc1cnc(Nc2ccc(N3CCN(C(=O)CCOCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- (2-((2-(2,6-dioxoisoindolin-4- yl)amino)ethoxy)propanoyl) piperazin-1-yl)phenyl)amino)- 5-methylpyrimidin-4- yl)amino)benzenesulfonamide |
| JA-018 | 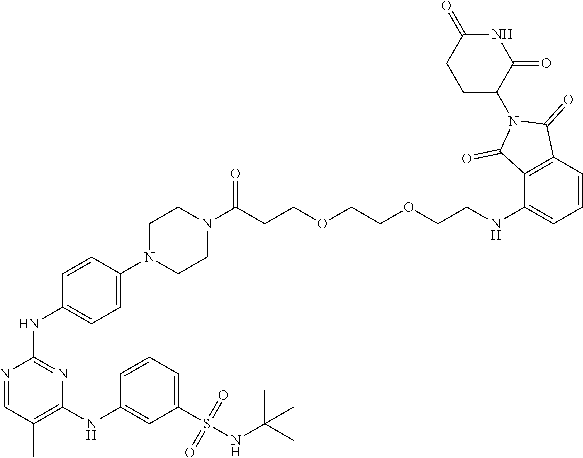 [SMILES: Cc1cnc(Nc2ccc(N3CCN(C(=O)CCOCCOCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- (2-(2-((2-(2,6- dioxopiperidin-3-yl)-1,3- dioxoisoindolin-4- yl)amino)ethoxy)ethoxy) propanoyl)piperazin-1- yl)phenyl)amino)-5- methylpyrimidin-4- yl)amino)benzenesulfonamide |
| JA-019 | 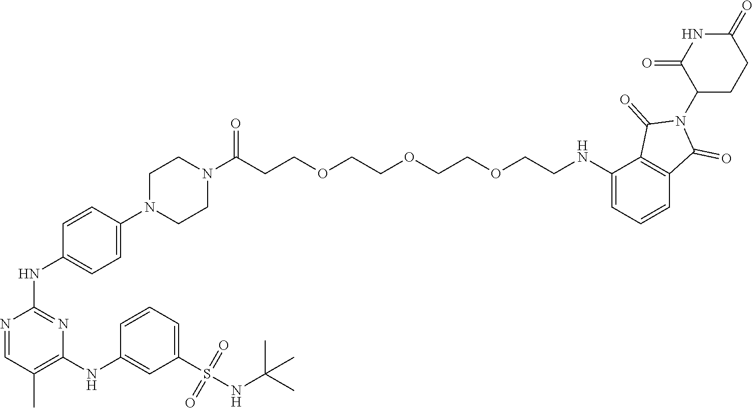 [SMILES: Cc1cnc(Nc2ccc(N3CCN(C(=O)CCOCCOCCOCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- (2-(2-(2-((2-(2,6- dioxopiperidin-3-yl)-1,3- dioxoisoindolin-4- yl)amino)ethoxy)ethoxy) ethoxy)propanoyl)piperazin-1- yl)phenyl)amino)-5- methylpyrimidin-4- yl)amino)benzenesulfonamide |
| JA-020 | 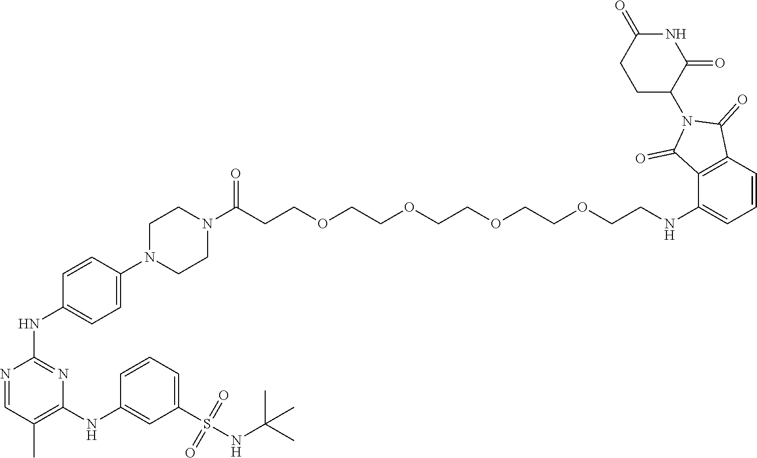 [SMILES: Cc1cnc(Nc2ccc(N3CCN(C(=O)CCOCCOCCOCCOCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(1- ((2-(2,6-dioxopiperidin-3-yl)- 1,3-dioxoisoindolin-4- yl)amino)-3,6,9,12- tetraoxapentadecan-15- oyl)piperazin-1- yl)phenyl)amino)-5- methylpyrimidin-4- yl)amino)benzenesulfonamide |
| JA-021 | 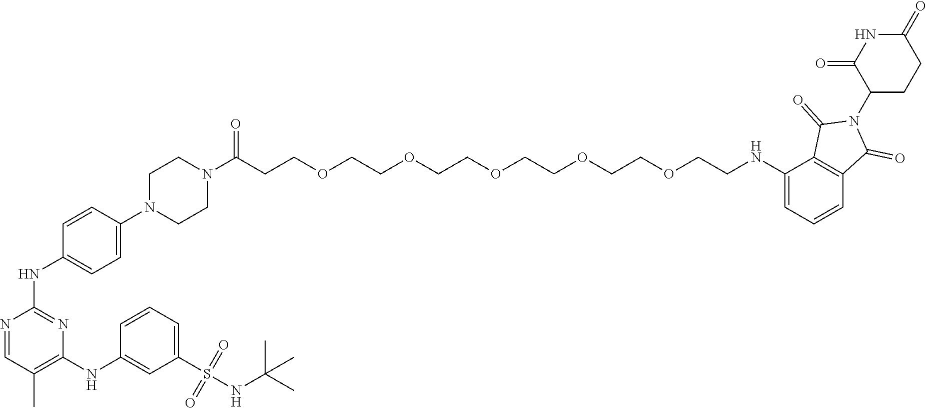 [SMILES: Cc1cnc(Nc2ccc(N3CCN(C(=O)CCOCCOCCOCCOCCOCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(1- ((2-(2,6-dioxopiperidin-3-yl)- 1,3-dioxoisoindolin-4- yl)amino)-3,6,9,12,15- pentaoxaoctadecan-18- oyl)piperazin-1- yl)phenyl)amino)-5- methylpyrimidin-4- yl)amino)benzenesulfonamide |
| JA-022 | 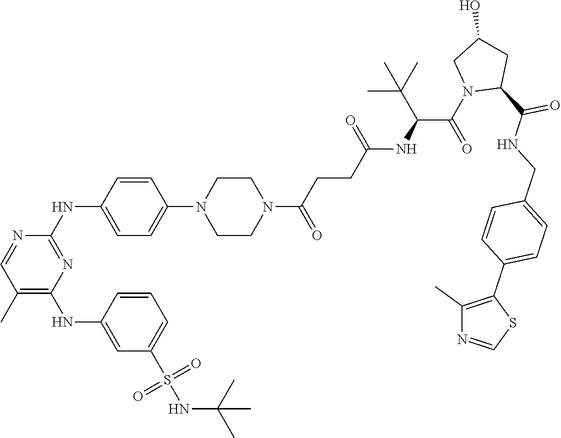 [SMILES: Cc1cnc(Nc2ccc(N3CCN(C(=O)CCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(4-(4-(4-((4- ((3-(N-(tert- butyl)sulfamoyl)phenyl) amino)-5-methylpyrimidin-2- yl)amino)phenyl)piperazin-1- yl)-4-oxobutanamido)-3,3- dimethylbutanoyl)-4- hydroxy-N-(4-(4- methylthiazol-5- yl)benzyl)pyrrolidine-2- carboxamide |
| JA-023 | 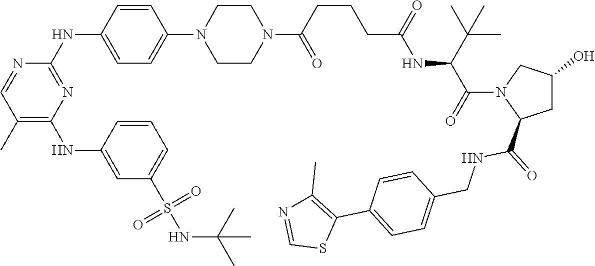 [SMILES: Cc1cnc(Nc2ccc(N3CCN(C(=O)CCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(5-(4-(4-((4- ((3-(N-(tert- butyl)sulfamoyl)phenyl) amino)-5-methylpyrimidin-2- yl)amino)phenyl)piperazin-1- yl)-5-oxopentanamido)-3,3- dimethylbutanoyl)-4- hydroxy-N-(4-(4- methylthiazol-5- yl)benzyl)pyrrolidine-2- carboxamide |
| JA-024 | 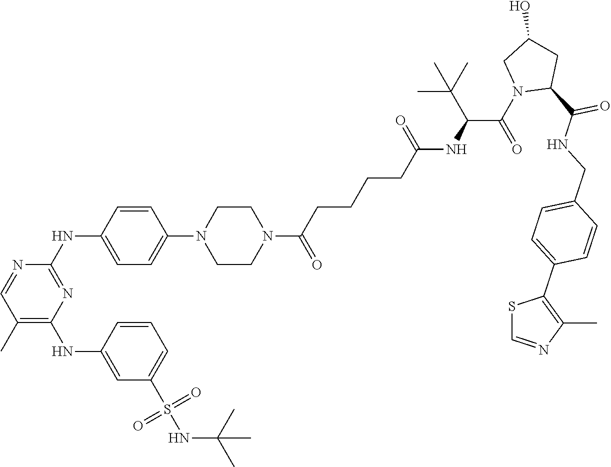 [SMILES: Cc1cnc(Nc2ccc(N3CCN(C(=O)CCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(6-(4-(4-((4- ((3-(N-(tert- butyl)sulfamoyl)phenyl) amino)-5-methylpyrimidin-2- yl)amino)phenyl)piperazin-1- yl)-6-oxohexanamido)-3,3- dimethylbutanoyl)-4- hydroxy-N-(4-(4- methylthiazol-5- yl)benzyl)pyrrolidine-2- carboxamide |
| JA-025 | 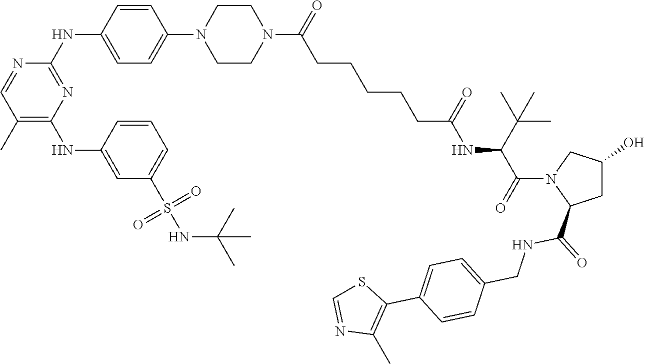 [SMILES: Cc1cnc(Nc2ccc(N3CCN(C(=O)CCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(7-(4-(4-((4- ((3-(N-(tert- butyl)sulfamoyl)phenyl) amino)-5-methylpyrimidin-2- yl)amino)phenyl)piperazin-1- yl)-7-oxoheptanamido)-3,3- dimethylbutanoyl)-4- hydroxy-N-(4-(4- methylthiazol-5- yl)benzyl)pyrrolidine-2- carboxamide |
| JA-026 | 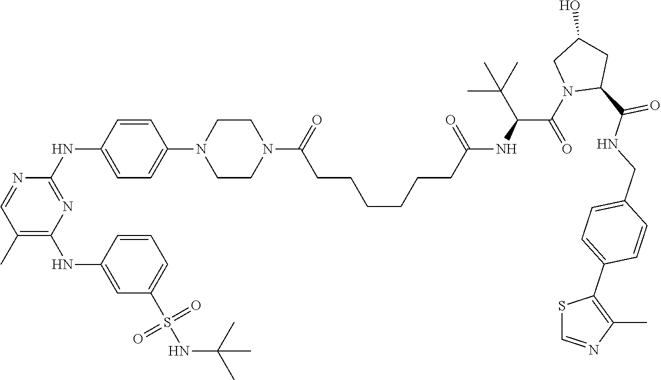 [SMILES: Cc1cnc(Nc2ccc(N3CCN(C(=O)CCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(8-(4-(4-((4- ((3-(N-(tert- butyl)sulfamoyl)phenyl) amino)-5-methylpyrimidin-2- yl)amino)phenyl)piperazin-1- yl)-8-oxooctanamido)-3,3- dimethylbutanoyl)-4- hydroxy-N-(4-(4- methylthiazol-5- yl)benzyl)pyrrolidine-2- carboxamide |
| JA-027 | 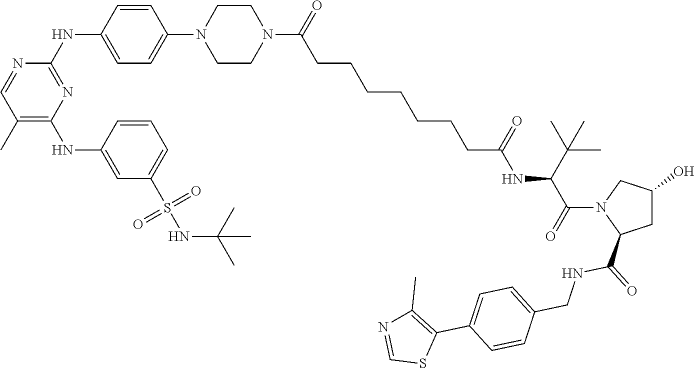 [SMILES: Cc1cnc(Nc2ccc(N3CCN(C(=O)CCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(9-(4-(4-((4- ((3-(N-(tert- butyl)sulfamoyl)phenyl) amino)-5-methylpyrimidin-2- yl)amino)phenyl)piperazin-1- yl)-9-oxononanamido)-3,3- dimethylbutanoyl)-4- hydroxy-N-(4-(4- methylthiazol-5- yl)benzyl)pyrrolidine-2- carboxamide |
| JA-028 | 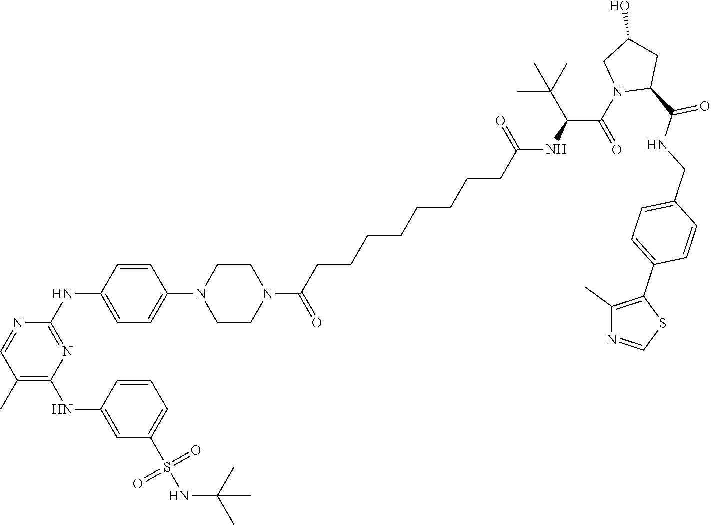 [SMILES: Cc1cnc(Nc2ccc(N3CCN(C(=O)CCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(10-(4-(4- ((4-((3-(N-(tert- butyl)sulfamoyl)phenyl) amino)-5-methylpyrimidin-2- yl)amino)phenyl)piperazin-1- yl)-10-oxodecanamido)-3,3- dimethylbutanoyl)-4- hydroxy-N-(4-(4- methylthiazol-5- yl)benzyl)pyrrolidine-2- carboxamide |
| JA-029 | 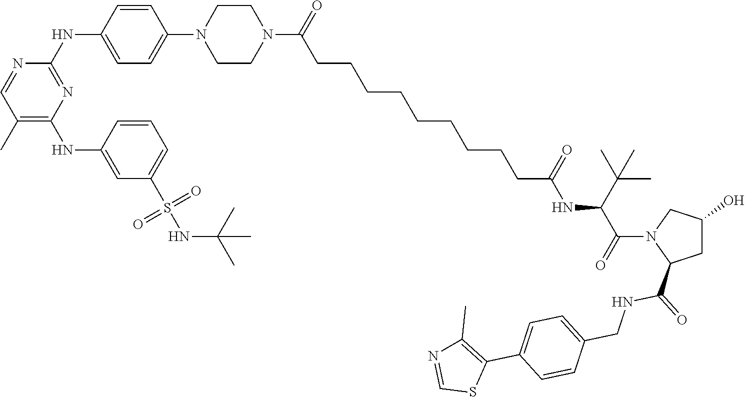 [SMILES: Cc1cnc(Nc2ccc(N3CCN(C(=O)CCCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(11-(4-(4- ((4-((3-(N-(tert- butyl)sulfamoyl)phenyl) amino)-5-methylpyrimidin-2- yl)amino)phenyl)piperazin-1- yl)-11-oxoundecanamido)- 3,3-dimethylbutanoyl)-4- hydroxy-N-(4-(4- methylthiazol-5- yl)benzyl)pyrrolidine-2- carboxamide |
| JA-030 | 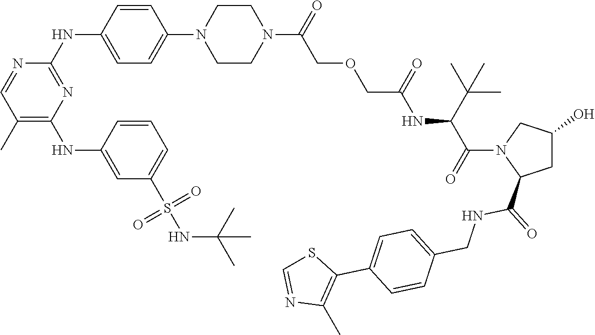 [SMILES: Cc1cnc(Nc2ccc(N3CCN(C(=O)COCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(2-(2-(4-(4- ((4-((3-(N-(tert- butyl)sulfamoyl)phenyl) amino)-5-methylpyrimidin-2- yl)amino)phenyl)piperazin-1- yl)-2-oxoethoxy)acetamido)- 3,3-dimethylbutanoyl)-4- hydroxy-N-(4-(4- methylthiazol-5- yl)benzyl)pyrrolidine-2- carboxamide |
| JA-031 | 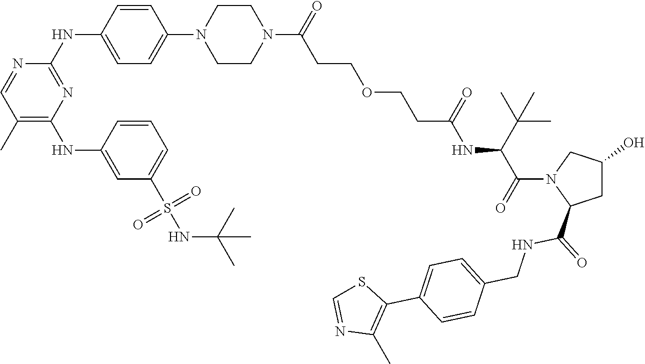 [SMILES: Cc1cnc(Nc2ccc(N3CCN(C(=O)CCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(3-(3-(4-(4- ((4-((3-(N-(tert- butyl)sulfamoyl)phenyl) amino)-5-methylpyrimidin-2- yl)amino)phenyl)piperazin-1- yl)-3- oxopropoxy)propanamido)- 3,3-dimethylbutanoyl)-4- hydroxy-N-(4-(4- methylthiazol-5- yl)benzyl)pyrrolidine-2- carboxamide |
| JA-032 | 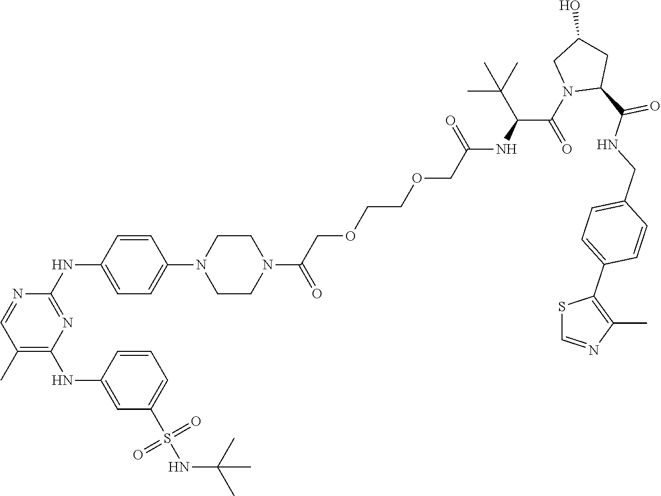 [SMILES: Cc1cnc(Nc2ccc(N3CCN(C(=O)COCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(2-(2-(2-(4- (4-((4-((3-(N-(tert- butyl)sulfamoyl)phenyl) amino)-5-methylpyrimidin-2- yl)amino)phenyl)piperazin-1- yl)-2- oxoethoxy)ethoxy)acetamido)- 3,3-dimethylbutanoyl)-4- hydroxy-N-(4-(4- methylthiazol-5- yl)benzyl)pyrrolidine-2- carboxamide |
| JA-033 | 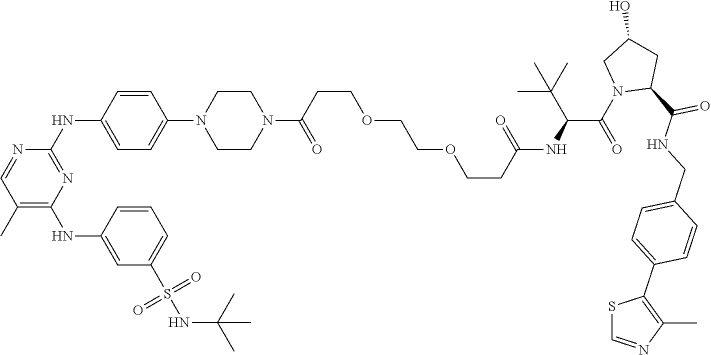 [SMILES: Cc1cnc(Nc2ccc(N3CCN(C(=O)CCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(3-(2-(3-(4- (4-((4-((3-(N-(tert- butyl)sulfamoyl)phenyl) amino)-5-methylpyrimidin-2- yl)amino)phenyl)piperazin-1- yl)-3- oxopropoxy)ethoxy) propanamido)-3,3- dimethylbutanoyl)-4- hydroxy-N-(4-(4- methylthiazol-5- yl)benzyl)pyrrolidine-2- carboxamide |
| JA-034 | 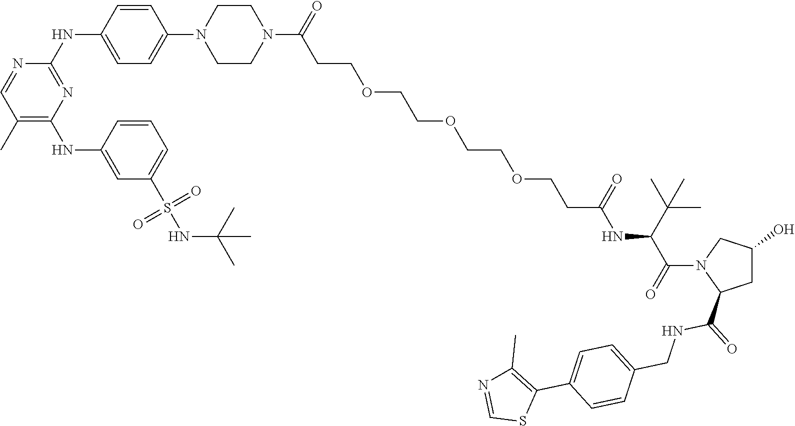 [SMILES: Cc1cnc(Nc2ccc(N3CCN(C(=O)CCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 16-(4-(4-((4-((3-(N-(tert- butyl)sulfamoyl)phenyl) amino)-5-methylpyrimidin-2- yl)amino)phenyl)piperazin-1- yl)-4,16-dioxo-7,10,13- trioxa-3-azahexadecanoyl)-4- hydroxy-N-(4-(4- methylthiazol-5- yl)benzyl)pyrrolidine-2- carboxamide |
| JA-035 | 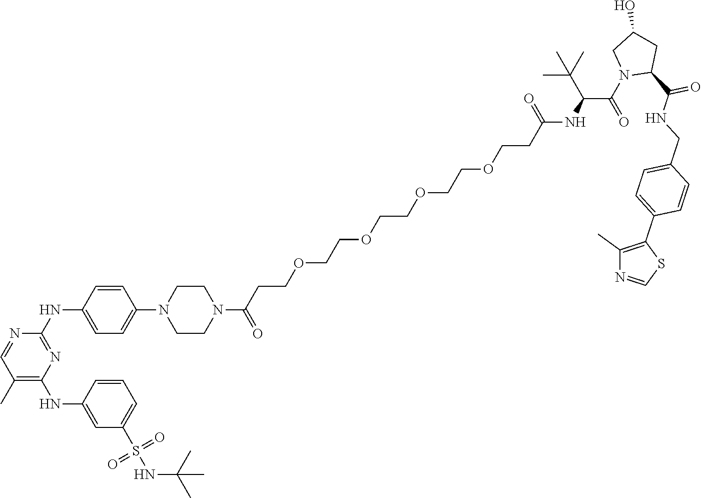 [SMILES: Cc1cnc(Nc2ccc(N3CCN(C(=O)CCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 19-(4-(4-((4-((3-(N-(tert- butyl)sulfamoyl)phenyl) amino)-5-methylpyrimidin-2- yl)amino)phenyl)piperazin-1- yl)-4,19-dioxo-7,10,13,16- tetraoxa-3-azanonadecanoyl)- 4-hydroxy-N-(4-(4- methylthiazol-5- yl)benzyl)pyrrolidine-2- carboxamide |
| JA-036 | 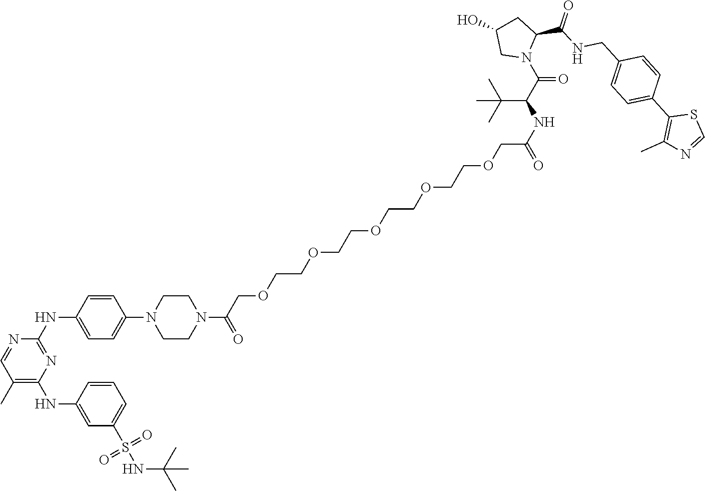 [SMILES: Cc1cnc(Nc2ccc(N3CCN(C(=O)COCCOCCOCCOCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 20-(4-(4-((4-((3-(N-(tert- butyl)sulfamoyl)phenyl) amino)-5-methylpyrimidin-2- yl)amino)phenyl)piperazin-1- yl)-4,20-dioxo-6,9,12,15,18- pentaoxa-3-azaicosanoyl)-4- hydroxy-N-(4-(4- methylthiazol-5- yl)benzyl)pyrrolidine-2- carboxamide |
| JA-037 | 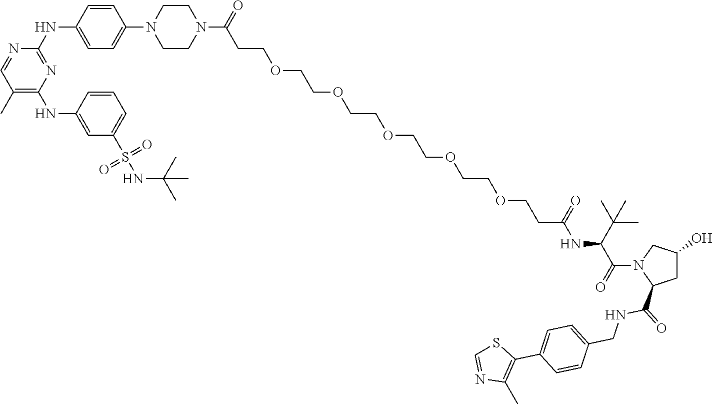 [SMILES: Cc1cnc(Nc2ccc(N3CCN(C(=O)CCOCCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 22-(4-(4-((4-((3-(N-(tert- butyl)sulfamoyl)phenyl) amino)-5-methylpyrimidin-2- yl)amino)phenyl)piperazin-1- yl)-4,22-dioxo- 7,10,13,16,19-pentaoxa-3- azadocosanoyl)-4-hydroxy- N-(4-(4-methylthiazol-5- yl)benzyl)pyrrolidine-2- carboxamide |
| JA-038 | 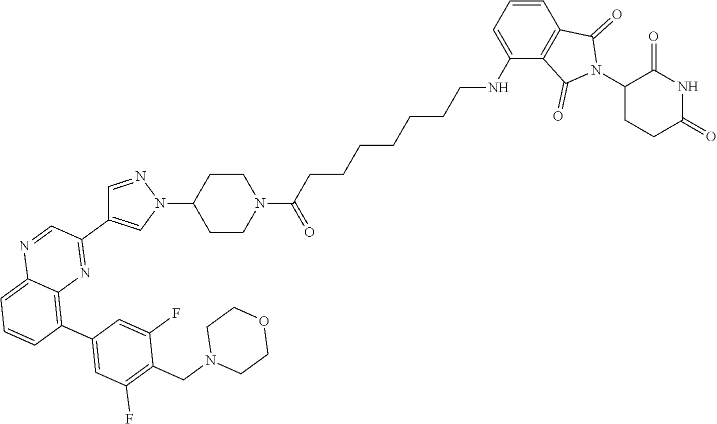 [SMILES: O=C1CCC(N2C(=O)c3cccc(NCCCCCCCC(=O)N4CCC(n5cc(-c6cnc7cccc(-c8cc(F)c(CN9CCOCC9)c(F)c8)c7n6)cn5)CC4)c3C2=O)C(=O)N1] | 4-[[8-[4-[4-[8-[3,5-difluoro- 4- (morpholinomethyl)phenyl] quinoxalin-2-yl]pyrazol-1-yl]- 1-piperidyl]-8-oxo- octyl]amino]-2-(2,6-dioxo-3- piperidyl)isoindoline-1,3- dione |
| JA-039 | 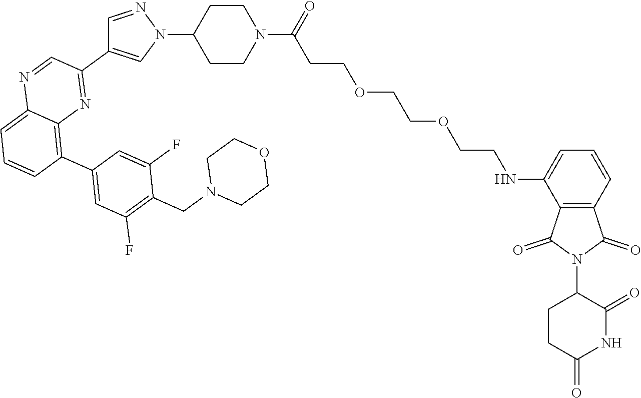 [SMILES: O=C1CCC(N2C(=O)c3cccc(NCCOCCOCCC(=O)N4CCC(n5cc(-c6cnc7cccc(-c8cc(F)c(CN9CCOCC9)c(F)c8)c7n6)cn5)CC4)c3C2=O)C(=O)N1] | 4-[2-[2-[3-[4-[4-[8-[3,5- difluoro-4- (morpholinomethyl)phenyl] quinoxalin-2-yl]pyrazol-1-yl]- 1-piperidyl]-3-oxo- propoxy]ethoxy]ethylamino]- 2-(2,6-dioxo-3- piperidyl)isoindoline-1,3- dione |
| JA-040 | 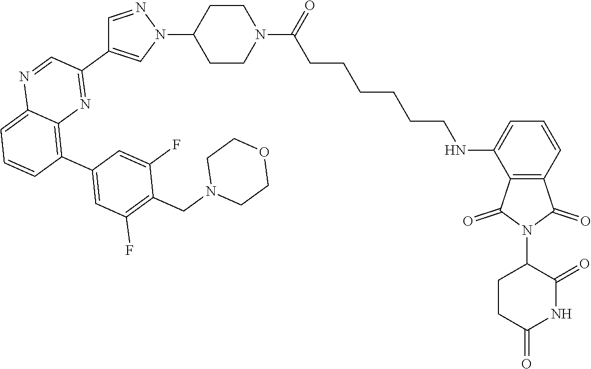 [SMILES: O=C1CCC(N2C(=O)c3cccc(NCCCCCCC(=O)N4CCC(n5cc(-c6cnc7cccc(-c8cc(F)c(CN9CCOCC9)c(F)c8)c7n6)cn5)CC4)c3C2=O)C(=O)N1] | 4-[[7-[4-[4-[8-[3,5-difluoro- 4- (morpholinomethyl)phenyl] quinoxalin-2-yl]pyrazol-1-yl]- 1-piperidyl]-7-oxo- heptyl]amino]-2-(2,6-dioxo- 3-piperidyl)isoindoline-1,3- dione |
| JA-041 | 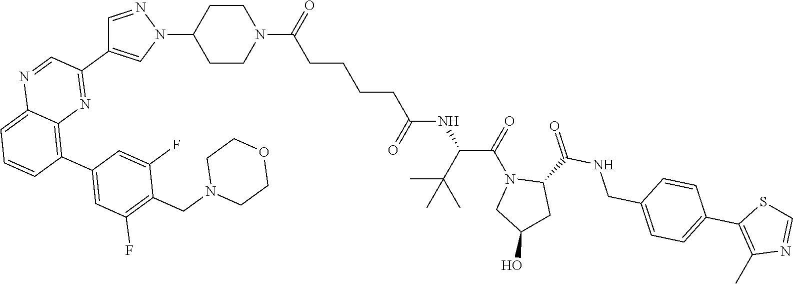 [SMILES: Cc1ncsc1-c1ccc(CNC(=O)[C@@H]2C[C@@H](O)CN2C(=O)[C@@H](NC(=O)CCCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[6-[4-[4- [8-[3,5-difluoro-4- (morpholinomethyl)phenyl] quinoxalin-2-yl]pyrazol-1-yl]- 1-piperidyl]-6-oxo- hexanoyl]amino]-3,3- dimethyl-butanoyl]-4- hydroxy-N-[[4-(4- methylthiazol-5- yl)phenyl]methyl]pyrrolidine- 2-carboxamide |
| JA-042 | 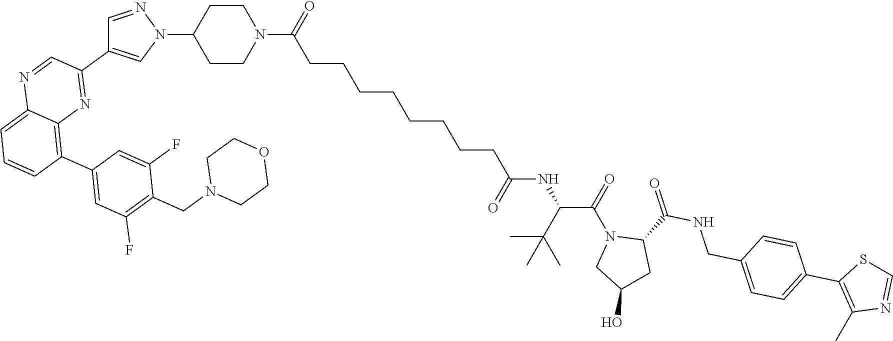 [SMILES: Cc1ncsc1-c1ccc(CNC(=O)[C@@H]2C[C@@H](O)CN2C(=O)[C@@H](NC(=O)CCCCCCCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[10-[4-[4- [8-[3,5-difluoro-4- (morpholinomethyl)phenyl] quinoxalin-2-yl]pyrazol-1-yl]- 1-piperidyl]-10-oxo- decanoyl]amino]-3,3- dimethyl-butanoyl]-4- hydroxy-N-[[4-(4- methylthiazol-5- yl)phenyl]methyl]pyrrolidine- 2-carboxamide |
| JA-043 | 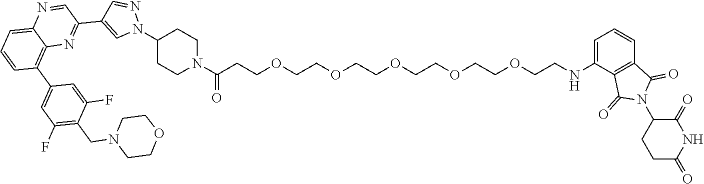 [SMILES: O=C1CCC(N2C(=O)c3cccc(NCCOCCOCCOCCOCCOCCC(=O)N4CCC(n5cc(-c6cnc7cccc(-c8cc(F)c(CN9CCOCC9)c(F)c8)c7n6)cn5)CC4)c3C2=O)C(=O)N1] | 4-[2-[2-[2-[2-[2-[3-[4-[4-[8- [3,5-difluoro-4- (morpholinomethyl)phenyl] quinoxalin-2-yl]pyrazol-1-yl]- 1-piperidyl]-3-oxo- propoxy]ethoxy]ethoxy] ethoxy]ethoxy]ethylamino]-2- (2,6-dioxo-3- piperidyl)isoindoline-1,3- dione |
| JA-044 | 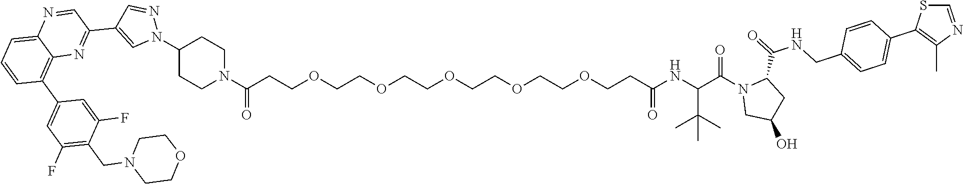 [SMILES: Cc1ncsc1-c1ccc(CNC(=O)[C@@H]2C[C@@H](O)CN2C(=O)C(NC(=O)CCOCCOCCOCCOCCOCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[3-[2-[2- [2-[2-[3-[4-[4-[8-[3,5- difluoro-4- (morpholinomethyl)phenyl] quinoxalin-2-yl]pyrazol-1-yl]- 1-piperidyl]-3-oxo- propoxy]ethoxy]ethoxy] ethoxy]ethoxy]propanoylamino]- 3,3-dimethyl-butanoyl]-4- hydroxy-N-[[4-(4- methylthiazol-5- yl)phenyl]methyl]pyrrolidine- |
| | | 2-carboxamide |
| JA-045 | 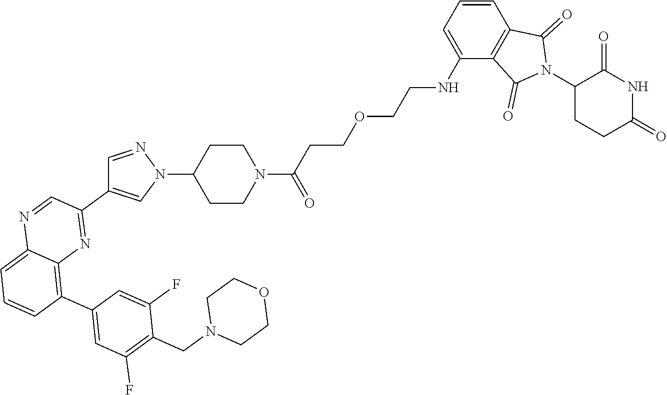 [SMILES: O=C1CCC(N2C(=O)c3cccc(NCCOCCC(=O)N4CCC(n5cc(-c6cnc7cccc(-c8cc(F)c(CN9CCOCC9)c(F)c8)c7n6)cn5)CC4)c3C2=O)C(=O)N1] | 4-[2-[3-[4-[4-[8-[3,5- difluoro-4- (morpholinomethyl)phenyl] quinoxalin-2-yl]pyrazol-1-yl]- 1-piperidyl]-3-oxo- propoxy]ethylamino]-2-(2,6- dioxo-3- piperidyl)isoindoline-1,3- dione |
| JA-046 | 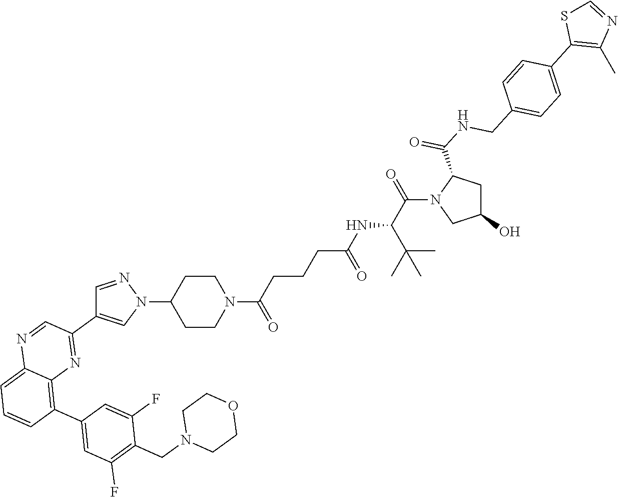 [SMILES: Cc1ncsc1-c1ccc(CNC(=O)[C@@H]2C[C@@H](O)CN2C(=O)[C@@H](NC(=O)CCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[5-[4-[4- [8-[3,5-difluoro-4- (morpholinomethyl)phenyl] quinoxalin-2-yl]pyrazol-1-yl]- 1-piperidyl]-5-oxo- pentanoyl]amino]-3,3- dimethyl-butanoyl]-4- hydroxy-N-[[4-(4- methylthiazol-5- yl)phenyl]methyl]pyrrolidine- 2-carboxamide |
| JA-047 | 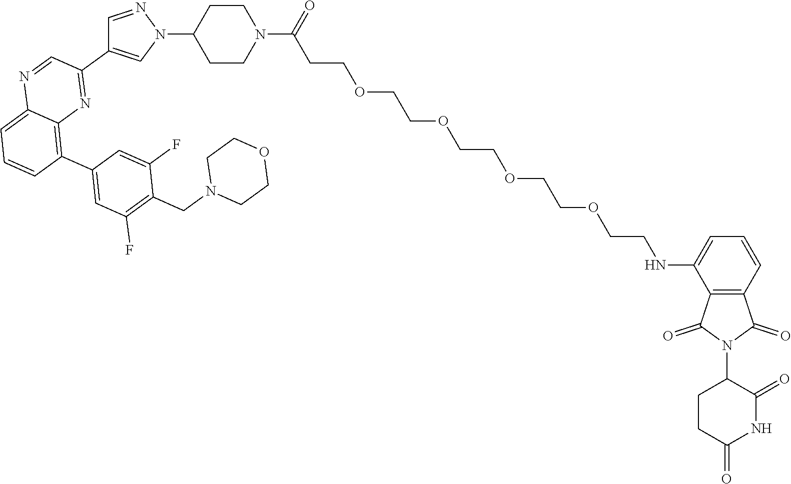 [SMILES: O=C1CCC(N2C(=O)c3cccc(NCCOCCOCCOCCOCCC(=O)N4CCC(n5cc(-c6cnc7cccc(-c8cc(F)c(CN9CCOCC9)c(F)c8)c7n6)cn5)CC4)c3C2=O)C(=O)N1] | 4-[2-[2-[2-[2-[3-[4-[4-[8- [3,5-difluoro-4- (morpholinomethyl)phenyl] quinoxalin-2-yl]pyrazol-1-yl]- 1-piperidyl]-3-oxo- propoxy]ethoxy]ethoxy] ethoxy]ethylamino]-2-(2,6-dioxo- 3-piperidyl)isoindoline-1,3- dione |
| JA-048 | 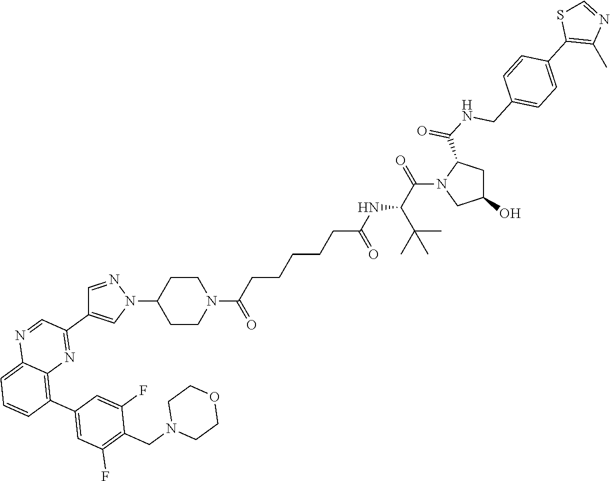 [SMILES: Cc1ncsc1-c1ccc(CNC(=O)[C@@H]2C[C@@H](O)CN2C(=O)[C@@H](NC(=O)CCCCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[7-[4-[4- [8-[3,5-difluoro-4- (morpholinomethyl)phenyl] quinoxalin-2-yl]pyrazol-1-yl]- 1-piperidyl]-7-oxo- heptanoyl]amino]-3,3- dimethyl-butanoyl]-4- hydroxy-N-[[4-(4- methylthiazol-5- yl)phenyl]methyl]pyrrolidine- 2-carboxamide |
| JA-049 | 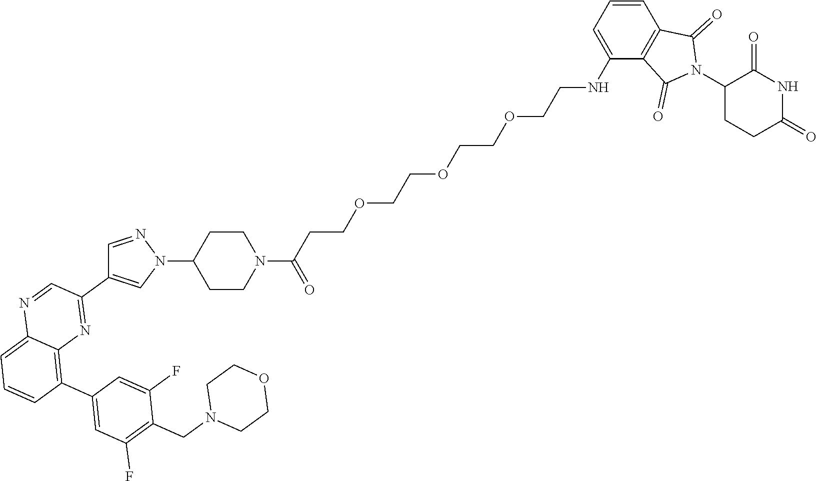 [SMILES: O=C1CCC(N2C(=O)c3cccc(NCCOCCOCCOCCC(=O)N4CCC(n5cc(-c6cnc7cccc(-c8cc(F)c(CN9CCOCC9)c(F)c8)c7n6)cn5)CC4)c3C2=O)C(=O)N1] | 4-[2-[2-[2-[3-[4-[4-[8-[3,5- difluoro-4- (morpholinomethyl)phenyl] quinoxalin-2-yl]pyrazol-1-yl]- 1-piperidyl]-3-oxo- propoxy]ethoxy]ethoxy]ethyl amino]-2-(2,6-dioxo-3- piperidyl)isoindoline-1,3- dione |
| JA-050 | 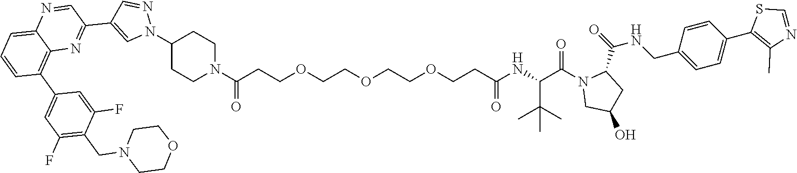 [SMILES: Cc1ncsc1-c1ccc(CNC(=O)[C@@H]2C[C@@H](O)CN2C(=O)[C@@H](NC(=O)CCOCCOCCOCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[3-[2-[2- [3-[4-[4-[8-[3,5-difluoro-4- (morpholinomethyl)phenyl] quinoxalin-2-yl]pyrazol-1-yl]- 1-piperidyl]-3-oxo- propoxy]ethoxy]ethoxy] propanoylamino]-3,3-dimethyl- butanoyl]-4-hydroxy-N-[[4- (4-methylthiazol-5- yl)phenyl]methyl]pyrrolidine- 2-carboxamide |
| JA-051 | 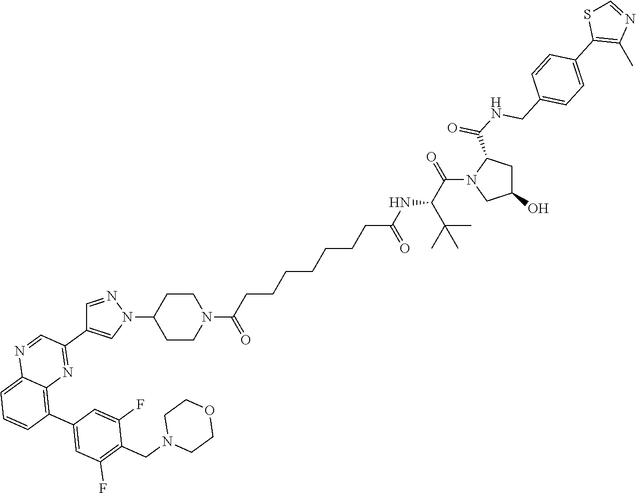 [SMILES: Cc1ncsc1-c1ccc(CNC(=O)[C@@H]2C[C@@H](O)CN2C(=O)[C@@H](NC(=O)CCCCCCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[9-[4-[4- [8-[3,5-difluoro-4- (morpholinomethyl)phenyl] quinoxalin-2-yl]pyrazol-1-yl]- 1-piperidyl]-9-oxo- nonanoyl]amino]-3,3- dimethyl-butanoyl]-4- hydroxy-N-[[4-(4- methylthiazol-5- yl)phenyl]methyl]pyrrolidine- 2-carboxamide |
| JA-052 | 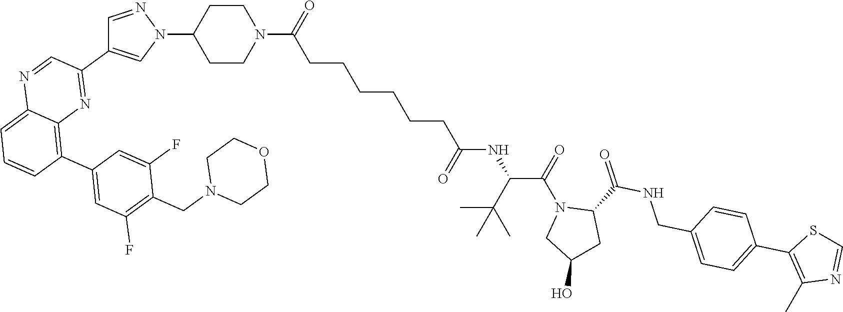 [SMILES: Cc1ncsc1-c1ccc(CNC(=O)[C@@H]2C[C@@H](O)CN2C(=O)[C@@H](NC(=O)CCCCCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[8-[4-[4- [8-[3,5-difluoro-4- (morpholinomethyl)phenyl] quinoxalin-2-yl]pyrazol-1-yl]- 1-piperidyl]-8-oxo- octanoyl]amino]-3,3- dimethyl-butanoyl]-4- hydroxy-N-[[4-(4- methylthiazol-5- yl)phenyl]methyl]pyrrolidine- 2-carboxamide |
| JA-053 | 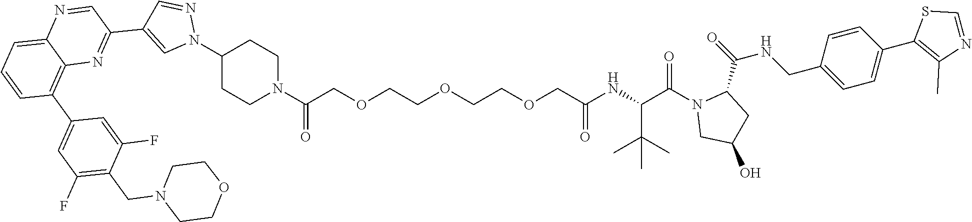 [SMILES: Cc1ncsc1-c1ccc(CNC(=O)[C@@H]2C[C@@H](O)CN2C(=O)[C@@H](NC(=O)COCCOCCOCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[2-[2-[2- [2-[4-[4-[8-[3,5-difluoro-4- (morpholinomethyl)phenyl] quinoxalin-2-yl]pyrazol-1-yl]- 1-piperidyl]-2-oxo- ethoxy]ethoxy]ethoxy]acetyl] amino]-3,3-dimethyl- butanoyl]-4-hydroxy-N-[[4- (4-methylthiazol-5- yl)phenyl]methyl]pyrrolidine- 2-carboxamide |
| JA-054 | 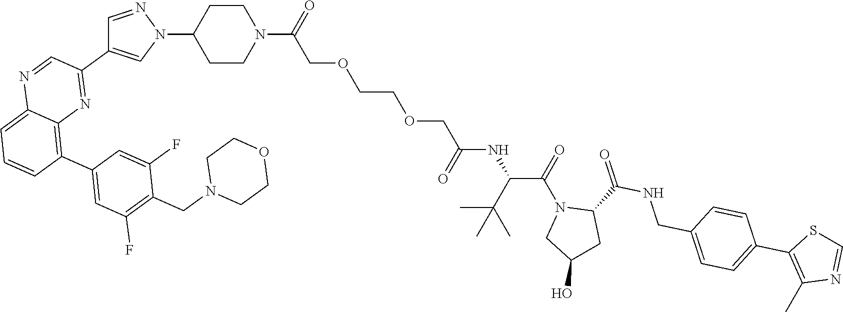 [SMILES: Cc1ncsc1-c1ccc(CNC(=O)[C@@H]2C[C@@H](O)CN2C(=O)[C@@H](NC(=O)COCCOCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[2-[2-[2- [4-[4-[8-[3,5-difluoro-4- (morpholinomethyl)phenyl] quinoxalin-2-yl]pyrazol-1-yl]- 1-piperidyl]-2-oxo- ethoxy]ethoxy]acetyl]amino]- 3,3-dimethyl-butanoyl]-4- hydroxy-N-[[4-(4- methylthiazol-5- yl)phenyl]methyl]pyrrolidine- 2-carboxamide |
| JA-055 | 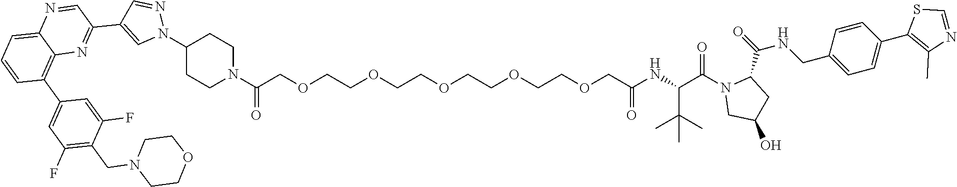 [SMILES: Cc1ncsc1-c1ccc(CNC(=O)[C@@H]2C[C@@H](O)CN2C(=O)[C@@H](NC(=O)COCCOCCOCCOCCOCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[2-[2-[2- [2-[2-[2-[4-[4-[8-[3,5- difluoro-4- (morpholinomethyl)phenyl] quinoxalin-2-yl]pyrazol-1-yl]- 1-piperidyl]-2-oxo- ethoxy]ethoxy]ethoxy]ethoxy] ethoxy]acetyl]amino]-3,3- dimethyl-butanoyl]-4- hydroxy-N-[[4-(4- methylthiazol-5- yl)phenyl]methyl]pyrrolidine- |
| | | 2-carboxamide |
| JA-056 | 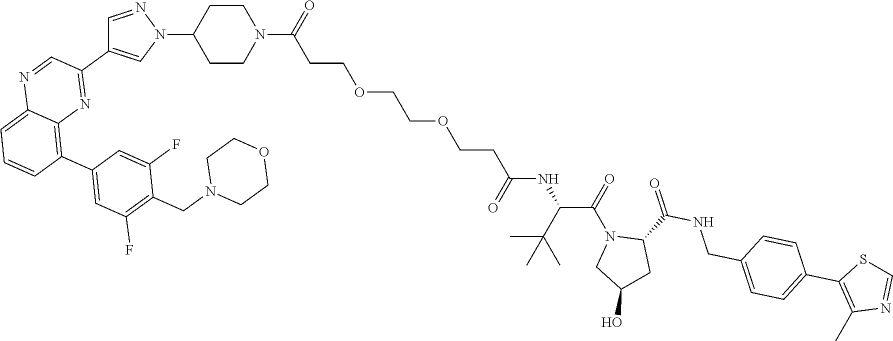 [SMILES: Cc1ncsc1-c1ccc(CNC(=O)[C@@H]2C[C@@H](O)CN2C(=O)[C@@H](NC(=O)CCOCCOCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[3-[2-[3- [4-[4-[8-[3,5-difluoro-4- (morpholinomethyl)phenyl] quinoxalin-2-yl]pyrazol-1-yl]- 1-piperidyl]-3-oxo- propoxy]ethoxy]propanoylamino]- 3,3-dimethyl- butanoyl]-4-hydroxy-N-[[4- (4-methylthiazol-5- yl)phenyl]methyl]pyrrolidine- 2-carboxamide |
| JA-057 | 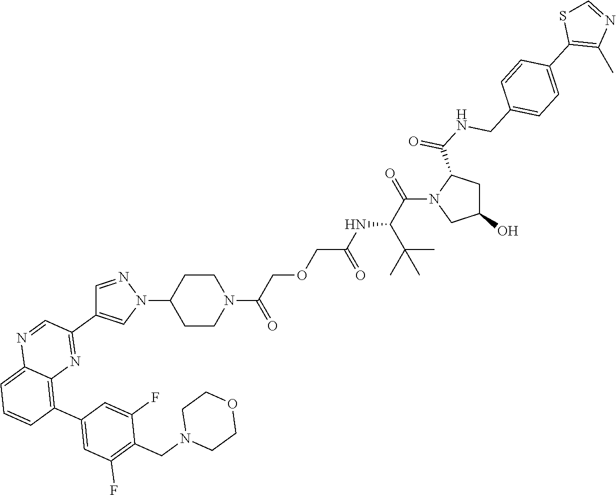 [SMILES: Cc1ncsc1-c1ccc(CNC(=O)[C@@H]2C[C@@H](O)CN2C(=O)[C@@H](NC(=O)COCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[2-[2-[4- [4-[8-[3,5-difluoro-4- (morpholinomethyl)phenyl] quinoxalin-2-yl]pyrazol-1-yl]- 1-piperidyl]-2-oxo- ethoxy]acetyl]amino]-3,3- dimethyl-butanoyl]-4- hydroxy-N-[[4-(4- methylthiazol-5- yl)phenyl]methyl]pyrrolidine- 2-carboxamide |
| JA-058 | 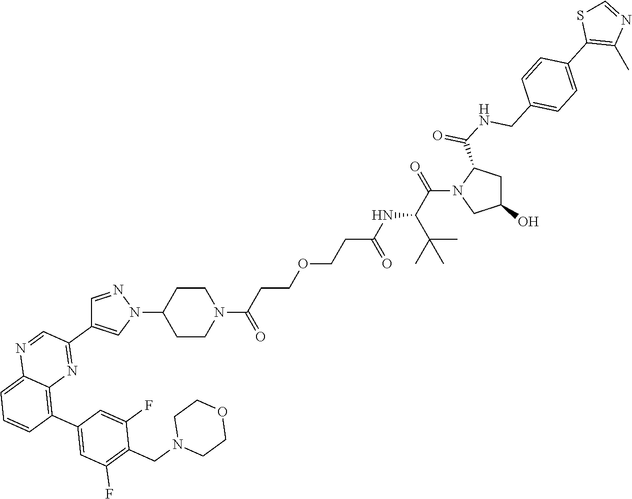 [SMILES: Cc1ncsc1-c1ccc(CNC(=O)[C@@H]2C[C@@H](O)CN2C(=O)[C@@H](NC(=O)CCOCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[3-[3-[4- [4-[8-[3,5-difluoro-4- (morpholinomethyl)phenyl] quinoxalin-2-yl]pyrazol-1-yl]- 1-piperidyl]-3-oxo- propoxy]propanoylamino]- 3,3-dimethyl-butanoyl]-4- hydroxy-N-[[4-(4- methylthiazol-5- yl)phenyl]methyl]pyrrolidine- 2-carboxamide |
| JA-059 | 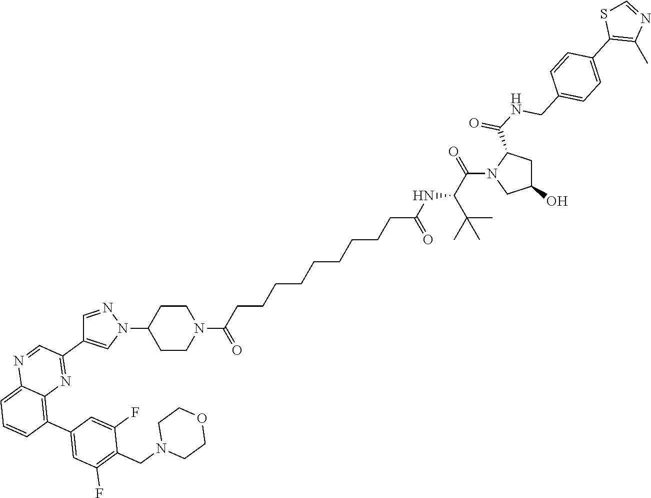 [SMILES: Cc1ncsc1-c1ccc(CNC(=O)[C@@H]2C[C@@H](O)CN2C(=O)[C@@H](NC(=O)CCCCCCCCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[11-[4-[4- [8-[3,5-difluoro-4- (morpholinomethyl)phenyl] quinoxalin-2-yl]pyrazol-1-yl]- 1-piperidyl]-11-oxo- undecanoyl]amino]-3,3- dimethyl-butanoyl]-4- hydroxy-N-[[4-(4- methylthiazol-5- yl)phenyl]methyl]pyrrolidine- 2-carboxamide |
| JA-060 | 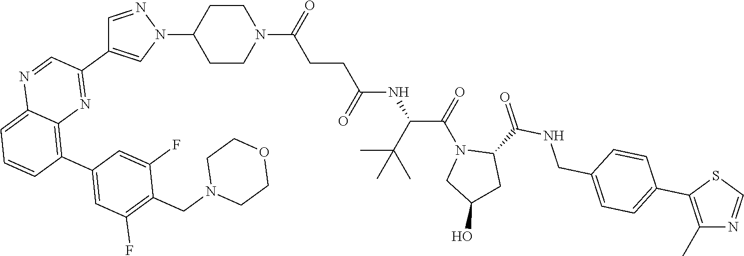 [SMILES: Cc1ncsc1-c1ccc(CNC(=O)[C@@H]2C[C@@H](O)CN2C(=O)[C@@H](NC(=O)CCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[[4-[4-[4- [8-[3,5-difluoro-4- (morpholinomethyl)phenyl] quinoxalin-2-yl]pyrazol-1-yl]- 1-piperidyl]-4-oxo- butanoyl]amino]-3,3- dimethyl-butanoyl]-4- hydroxy-N-[[4-(4- methylthiazol-5- yl)phenyl]methyl]pyrrolidine- 2-carboxamide |
| JA-061 | 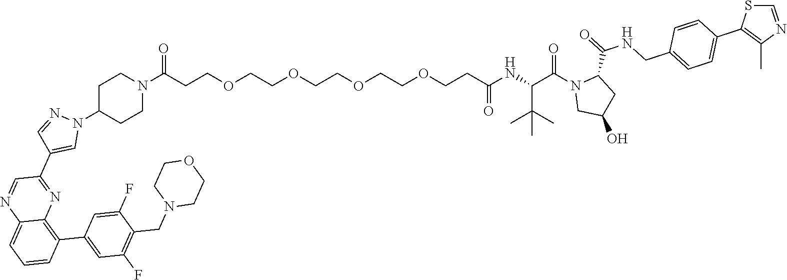 [SMILES: Cc1ncsc1-c1ccc(CNC(=O)[C@@H]2C[C@@H](O)CN2C(=O)[C@@H](NC(=O)CCOCCOCCOCCOCCC(=O)N2CCC(n3cc(-c4cnc5cccc(-c6cc(F)c(CN7CCOCC7)c(F)c6)c5n4)cn3)CC2)C(C)(C)C)cc1] | (2S,4R)-1-[(2S)-2-[3-[2-[2- [2-[3-[4-[4-[8-[3,5-difluoro- 4- (morpholinomethyl)phenyl] quinoxalin-2-yl]pyrazol-1-yl]- 1-piperidyl]-3-oxo- propoxy]ethoxy] ethoxy]ethoxy]propanoylamino]- 3,3-dimethyl-butanoyl]- 4-hydroxy-N-[[4-(4- methylthiazol-5- yl)phenyl]methyl] pyrrolidine-2-carboxamide |
| JA-062 | 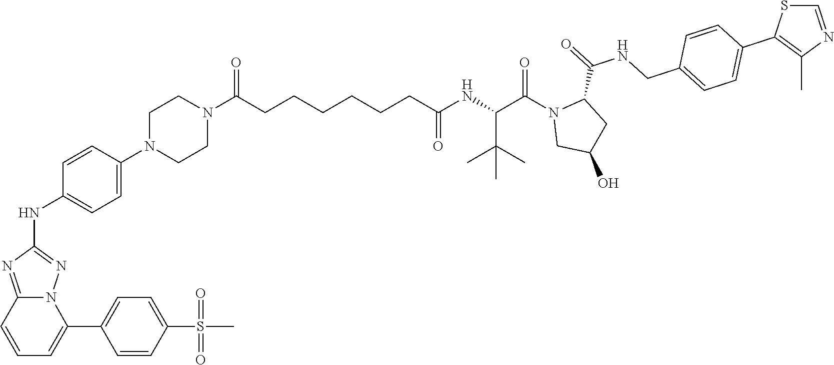 [SMILES: Cc1ncsc1-c1ccc(CNC(=O)[C@@H]2C[C@@H](O)CN2C(=O)[C@@H](NC(=O)CCCCCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(8-(4-(4-((5-(4- (methylsulfonyl)phenyl)- [1,2,4]triazolo[1,5-a]pyridin- 2-yl)amino)phenyl)piperazin- 1-yl)-8- oxooctanamido)butanoyl)-4- hydroxy-N-(4-(4- methylthiazol-5- yl)benzyl)pyrrolidine-2- carboxamide |
| JA-063 | 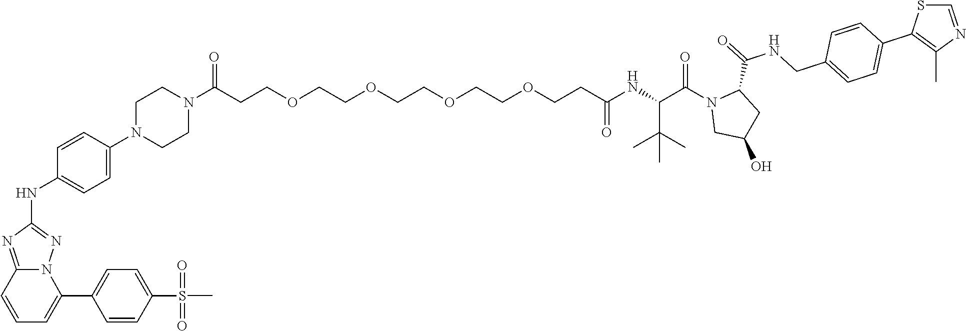 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 19-(4-(4-((5-(4- (methylsulfonyl)phenyl)- [1,2,4]triazolo[1,5-a]pyridin- 2-yl)amino)phenyl)piperazin- 1-yl)-4,19-dioxo-7,10,13,16- tetraoxa-3-azanonadecanoyl)- 4-hydroxy-N-(4-(4- methylthiazol-5- yl)benzyl)pyrrolidine-2- carboxamide |
| JA-064 | 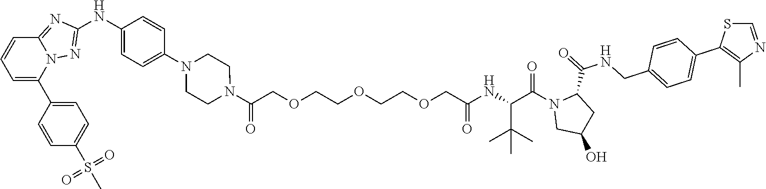 [SMILES: Cc1ncsc1-c1ccc(CNC(=O)[C@@H]2C[C@@H](O)CN2C(=O)[C@@H](NC(=O)COCCOCCOCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 14-(4-(4-((5-(4- (methylsulfonyl)phenyl)- [1,2,4]triazolo[1,5-a]pyridin- 2-yl)amino)phenyl)piperazin- 1-yl)-4,14-dioxo-6,9,12- trioxa-3-azatetradecanoyl)-4- hydroxy-N-(4-(4- methylthiazol-5- yl)benzyl)pyrrolidine-2- carboxamide |
| JA-065 | 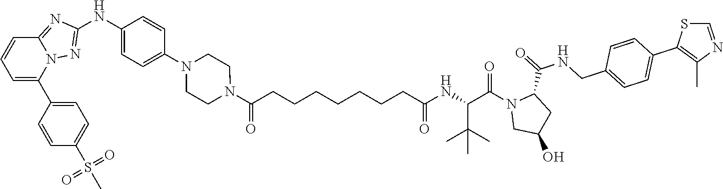 [SMILES: Cc1ncsc1-c1ccc(CNC(=O)[C@@H]2C[C@@H](O)CN2C(=O)[C@@H](NC(=O)CCCCCCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(9-(4-(4-((5-(4- (methylsulfonyl)phenyl)- [1,2,4]triazolo[1,5-a]pyridin- 2-yl)amino)phenyl)piperazin- 1-yl)-9- oxononanamido)butanoyl)-4- hydroxy-N-(4-(4- methylthiazol-5- yl)benzyl)pyrrolidine-2- carboxamide |
| JA-066 | 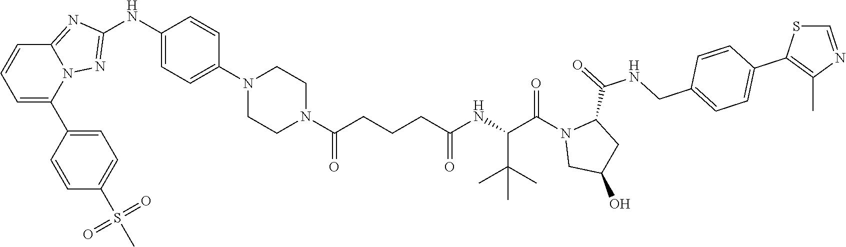 [SMILES: Cc1ncsc1-c1ccc(CNC(=O)[C@@H]2C[C@@H](O)CN2C(=O)[C@@H](NC(=O)CCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(5-(4-(4-((5-(4- (methylsulfonyl)phenyl)- [1,2,4]triazolo[1,5-a]pyridin- 2-yl)amino)phenyl)piperazin- 1-yl)-5- oxopentanamido)butanoyl)- 4-hydroxy-N-(4-(4- methylthiazol-5- yl)benzyl)pyrrolidine-2- carboxamide |
| JA-067 | 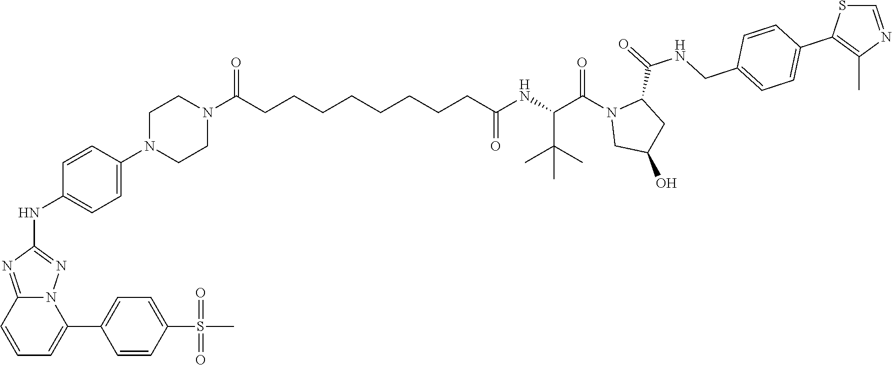 [SMILES: Cc1ncsc1-c1ccc(CNC(=O)[C@@H]2C[C@@H](O)CN2C(=O)[C@@H](NC(=O)CCCCCCCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(10-(4-(4-((5-(4- (methylsulfonyl)phenyl)- [1,2,4]triazolo[1,5-a]pyridin- 2-yl)amino)phenyl)piperazin- 1-yl)-10- oxodecanamido)butanoyl)-4- hydroxy-N-(4-(4- methylthiazol-5- yl)benzyl)pyrrolidine-2- carboxamide |
| JA-068 | 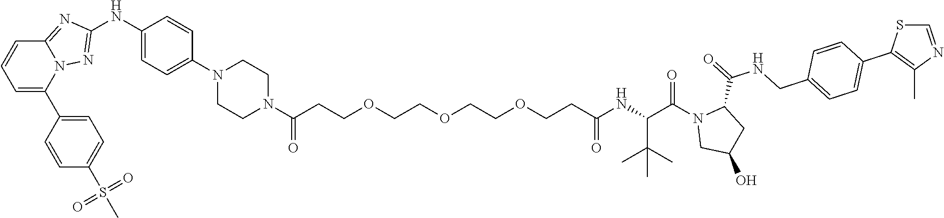 [SMILES: Cc1ncsc1-c1ccc(CNC(=O)[C@@H]2C[C@@H](O)CN2C(=O)[C@@H](NC(=O)CCOCCOCCOCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 16-(4-(4-((5-(4- (methylsulfonyl)phenyl)- [1,2,4]triazolo[1,5-a]pyridin- 2-yl)amino)phenyl)piperazin- 1-yl)-4,16-dioxo-7,10,13- trioxa-3-azahexadecanoyl)-4- hydroxy-N-(4-(4- methylthiazol-5- yl)benzyl)pyrrolidine-2- carboxamide |
| JA-069 | 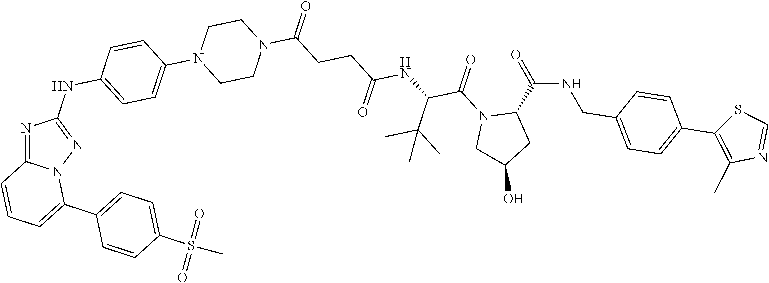 [SMILES: Cc1ncsc1-c1ccc(CNC(=O)[C@@H]2C[C@@H](O)CN2C(=O)[C@@H](NC(=O)CCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(4-(4-(4-((5-(4- (methylsulfonyl)phenyl)- [1,2,4]triazolo[1,5-a]pyridin- 2-yl)amino)phenyl)piperazin- 1-yl)-4- oxobutanamido)butanoyl)-4- hydroxy-N-(4-(4- methylthiazol-5- yl)benzyl)pyrrolidine-2- carboxamide |
| JA-070 | 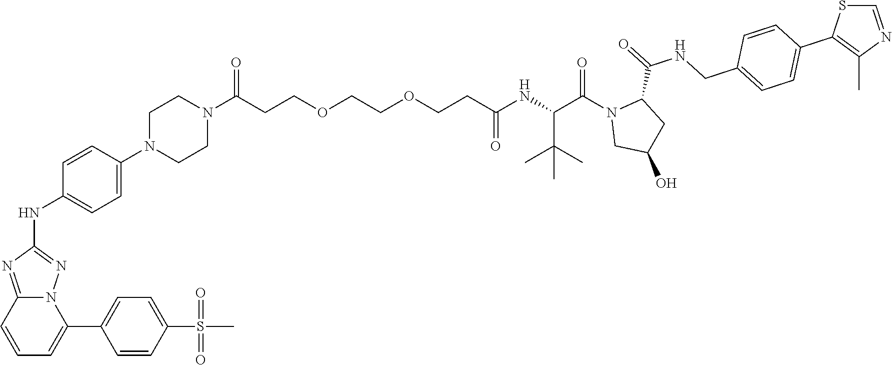 [SMILES: Cc1ncsc1-c1ccc(CNC(=O)[C@@H]2C[C@@H](O)CN2C(=O)[C@@H](NC(=O)CCOCCOCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(3-(2-(3-(4-(4-((5-(4- (methylsulfonyl)phenyl)- [1,2,4]triazolo[1,5-a]pyridin- 2-yl)amino)phenyl)piperazin- 1-yl)-3- oxopropoxy)ethoxy)propanamido) butanoyl)-4-hydroxy- N-(4-(4-methylthiazol-5- yl)benzyl)pyrrolidine-2- carboxamide |
| JA-071 | 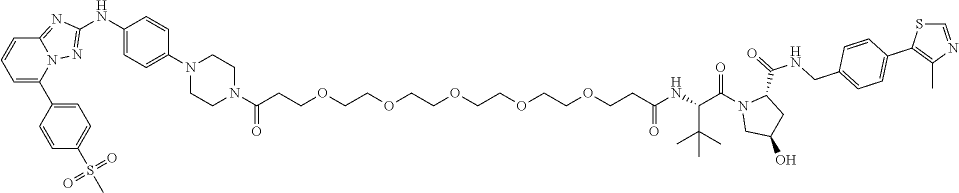 [SMILES: Cc1ncsc1-c1ccc(CNC(=O)[C@@H]2C[C@@H](O)CN2C(=O)[C@@H](NC(=O)CCOCCOCCOCCOCCOCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 22-(4-(4-((5-(4- (methylsulfonyl)phenyl)- [1,2,4]triazolo[1,5-a]pyridin- 2-yl)amino)phenyl)piperazin- 1-yl)-4,22-dioxo- 7,10,13,16,19-pentaoxa-3- azadocosoanoyl)-4-hydroxy- N-(4-(4-methylthiazol-5- yl)benzyl)pyrrolidine-2- carboxamide |
| JA-072 | 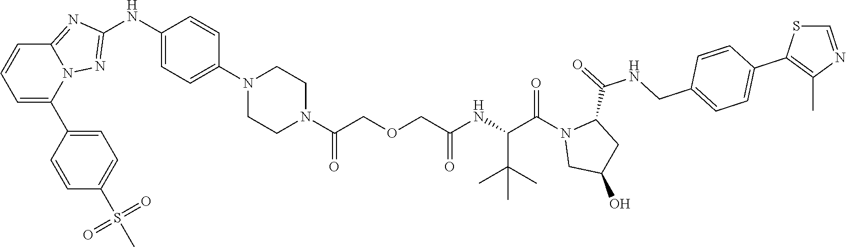 [SMILES: Cc1ncsc1-c1ccc(CNC(=O)[C@@H]2C[C@@H](O)CN2C(=O)[C@@H](NC(=O)COCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(2-(2-(4-(4-((5-(4- (methylsulfonyl)phenyl)- [1,2,4]triazolo[1,5-a]pyridin- 2-yl)amino)phenyl)piperazin- 1-yl)-2- oxoethoxy)acetamido)butanoyl)- 4-hydroxy-N-(4-(4- methylthiazol-5- yl)benzyl)pyrrolidine-2- carboxamide |
| JA-073 | 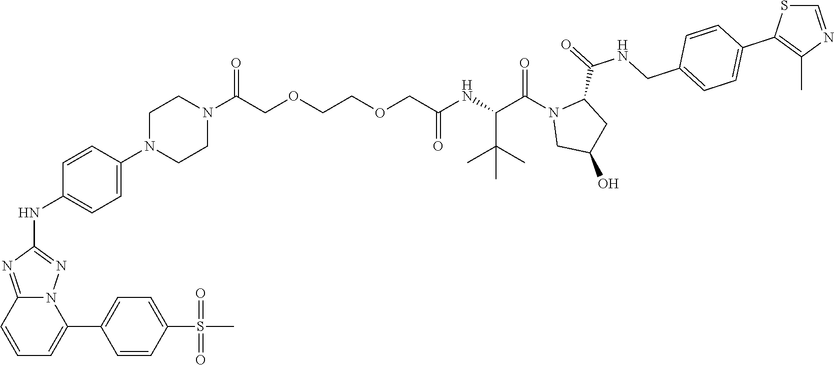 [SMILES: Cc1ncsc1-c1ccc(CNC(=O)[C@@H]2C[C@@H](O)CN2C(=O)[C@@H](NC(=O)COCCOCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(2-(2-(4-(4-((5-(4- (methylsulfonyl)phenyl)- [1,2,4]triazolo[1,5-a]pyridin- 2-yl)amino)phenyl)piperazin- 1-yl)-2- oxoethoxy)ethoxy)acetamido) butanoyl)-4-hydroxy-N-(4- (4-methylthiazol-5- yl)benzyl)pyrrolidine-2- carboxamide |
| JA-074 | 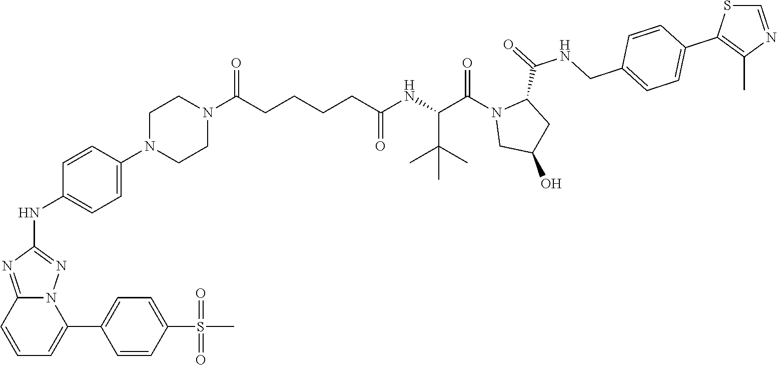 [SMILES: Cc1ncsc1-c1ccc(CNC(=O)[C@@H]2C[C@@H](O)CN2C(=O)[C@@H](NC(=O)CCCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(6-(4-(4-((5-(4- (methylsulfonyl)phenyl)- [1,2,4]triazolo[1,5-a]pyridin- 2-yl)amino)phenyl)piperazin- 1-yl)-6- oxohexanamido)butanoyl)-4- hydroxy-N-(4-(4- methylthiazol-5- yl)benzyl)pyrrolidine-2- carboxamide |
| JA-075 | 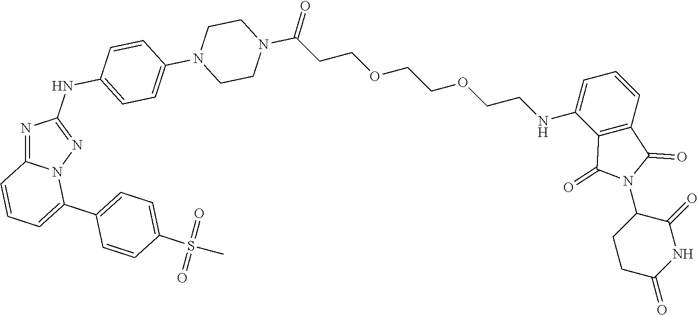 [SMILES: CS(=O)(=O)c1ccc(-c2cccc3nc(Nc4ccc(N5CCN(C(=O)CCOCCOCCNc6cccc7c6C(=O)N(C6CCC(=O)NC6=O)C7=O)CC5)cc4)nn23)cc1] | 2-(2,6-dioxopiperidin-3-yl)- 4-((2-(2-(3-(4-(4-((5-(4- (methylsulfonyl)phenyl)- [1,2,4]triazolo[1,5-a]pyridin- 2-yl)amino)phenyl)piperazin- 1-yl)-3- oxopropoxy)ethoxy)ethyl)amino) isoindoline-1,3-dione |
| JA-076 | 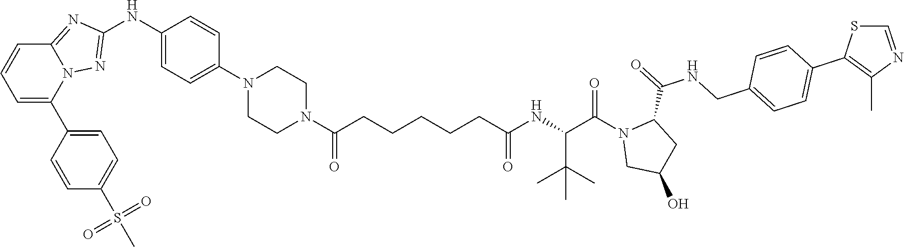 [SMILES: Cc1ncsc1-c1ccc(CNC(=O)[C@@H]2C[C@@H](O)CN2C(=O)[C@@H](NC(=O)CCCCCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(7-(4-(4-((5-(4- (methylsulfonyl)phenyl)- [1,2,4]triazolo[1,5-a]pyridin- 2-yl)amino)phenyl)piperazin- 1-yl)-7- oxoheptanamido)butanoyl)- 4-hydroxy-N-(4-(4- methylthiazol-5- yl)benzyl)pyrrolidine-2- carboxamide |
| JA-077 | 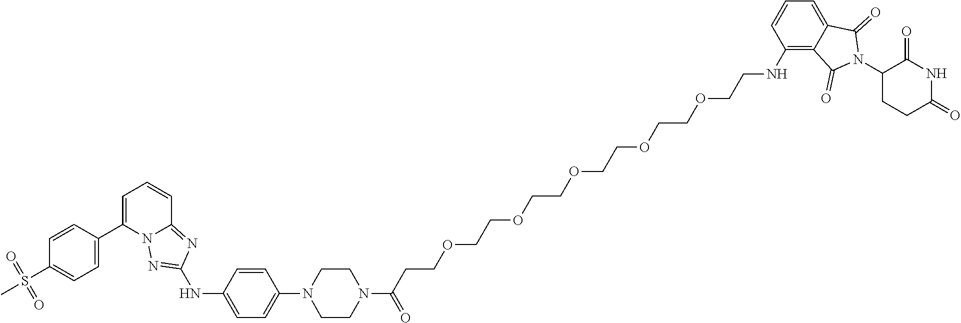 [SMILES: CS(=O)(=O)c1ccc(-c2cccc3nc(Nc4ccc(N5CCN(C(=O)CCOCCOCCOCCOCCOCCNc6cccc7c6C(=O)N(C6CCC(=O)NC6=O)C7=O)CC5)cc4)nn23)cc1] | 2-(2,6-Dioxo-piperidin-3-yl)- 4-(2-{2-[2-(2-{2-[3-(4-{4-[5- (4-methanesulfonyl-phenyl)- [1,2,4]triazolo[1,5-a]pyridin- 2-ylamino]-phenyl}- piperazin-1-yl)-3-oxo- propoxy]-ethoxy}-ethoxy)- ethoxy]-ethoxy}- ethylamino)-isoindole-1,3- dione |
| JA-078 | 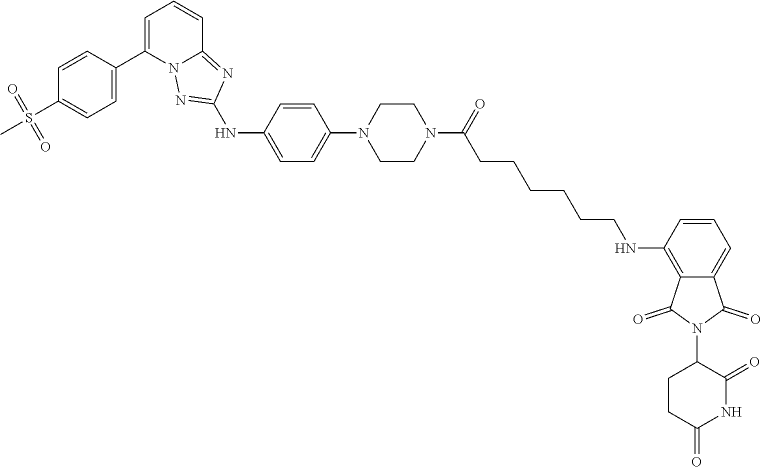 [SMILES: CS(=O)(=O)c1ccc(-c2cccc3nc(Nc4ccc(N5CCN(C(=O)CCCCCCNc6cccc7c6C(=O)N(C6CCC(=O)NC6=O)C7=O)CC5)cc4)nn23)cc1] | 2-(2,6-Dioxo-piperidin-3-yl)- 4-[7-(4-{4-[5-(4- methanesulfonyl-phenyl)- [1,2,4]triazolo[1,5-a]pyridin- 2-ylamino]-phenyl}- piperazin-1-yl)-7-oxo- heptylamino]-isoindole-1,3- dione |
| JA-079 | 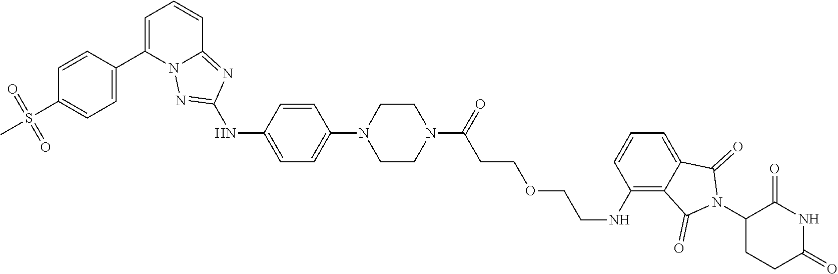 [SMILES: CS(=O)(=O)c1ccc(-c2cccc3nc(Nc4ccc(N5CCN(C(=O)CCOCCNc6cccc7c6C(=O)N(C6CCC(=O)NC6=O)C7=O)CC5)cc4)nn23)cc1] | 2-(2,6-Dioxo-piperidin-3-yl)- 4-{2-[3-(4-{4-[5-(4- methanesulfonyl-phenyl)- [1,2,4]triazolo[1,5-a]pyridin- 2-ylamino]-phenyl}- piperazin-1-yl)-3-oxo- propoxy]-ethylamino}- isoindole-1,3-dione |
| JA-080 | 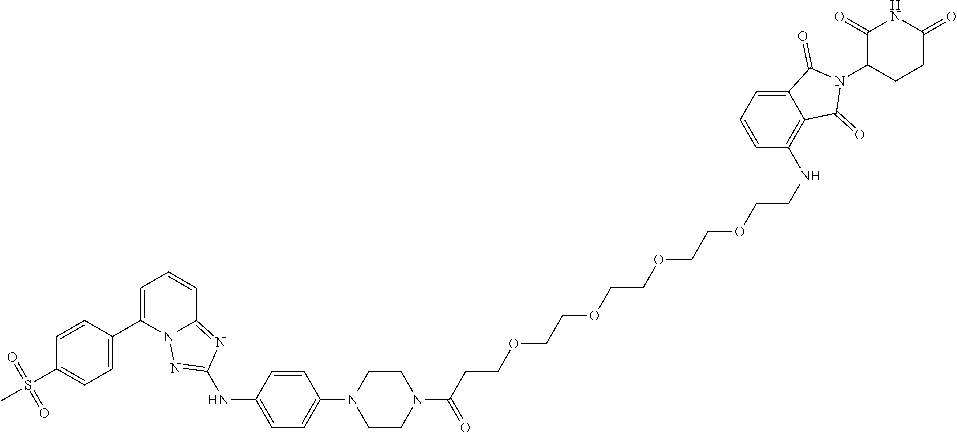 [SMILES: CS(=O)(=O)c1ccc(-c2cccc3nc(Nc4ccc(N5CCN(C(=O)CCOCCOCCOCCOCCNc6cccc7c6C(=O)N(C6CCC(=O)NC6=O)C7=O)CC5)cc4)nn23)cc1] | 2-(2,6-Dioxo-piperidin-3-yl)- 4-{2-[2-(2-{2-[3-(4-{4-[5-(4- methanesulfonyl-phenyl)- [1,2,4]triazolo[1,5-a]pyridin- 2-ylamino]-phenyl}- piperazin-1-yl)-3-oxo- propoxy]-ethoxy}-ethoxy)- ethoxy]-ethylamino}- isoindole-1,3-dione |
| JA-081 | 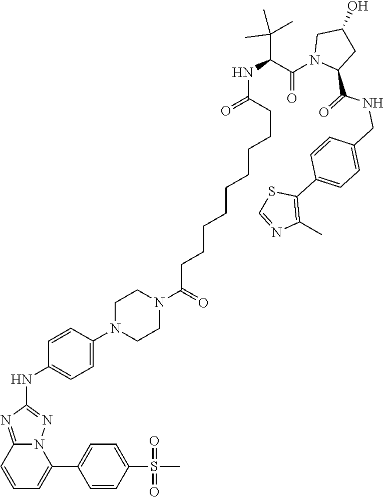 [SMILES: Cc1ncsc1-c1ccc(CNC(=O)[C@@H]2C[C@@H](O)CN2C(=O)[C@@H](NC(=O)CCCCCCCCCC(=O)N2CCN(c3ccc(Nc4nc5cccc(-c6ccc(S(C)(=O)=O)cc6)n5n4)cc3)CC2)C(C)(C)C)cc1] | 4-Hydroxy-1-{2-[11-(4-{4- [5-(4-methanesulfonyl- phenyl)-[1,2,4]triazolo[1,5- a]pyridin-2-ylamino]- phenyl}-piperazin-1-yl)-11- oxo-undecanoylamino]-3,3- dimethyl-butyryl}- pyrrolidine-2-carboxylic acid 4-(4-methyl-thiazol-5-yl)- benzylamide |
| JA-082 | 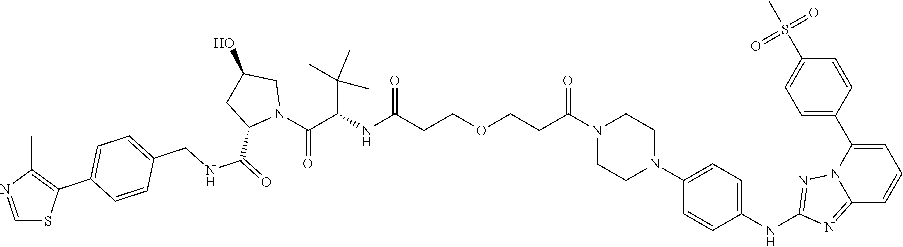 [SMILES: Cc1ncsc1-c1ccc(CNC(=O)[C@@H]2C[C@@H](O)CN2C(=O)[C@@H](NC(=O)CCOCCC(=O)N2CCN(c3ccc(Nc4nc5cccc(-c6ccc(S(C)(=O)=O)cc6)n5n4)cc3)CC2)C(C)(C)C)cc1] | 4-Hydroxy-1-(2-{3-[3-(4-{4- [5-(4-methanesulfonyl- phenyl)-[1,2,4]triazolo[1,5- a]pyridin-2-ylamino]- phenyl}-piperazin-1-yl)-3- oxo-propoxy]- propionylamino}-3,3- dimethyl-butyryl)- pyrrolidine-2-carboxylic acid 4-(4-methyl-thiazol-5-yl)- benzylamide |
| JA-083 | 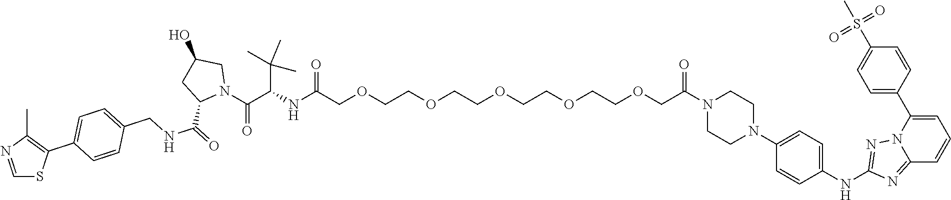 [SMILES: Cc1ncsc1-c1ccc(CNC(=O)[C@@H]2C[C@@H](O)CN2C(=O)[C@@H](NC(=O)COCCOCCOCCOCCOCC(=O)N2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 20-(4-(4-((5-(4- (methylsulfonyl)phenyl)- [1,2,4]triazolo[1,5-a]pyridin- 2-yl)amino)phenyl)piperazin- 1-yl)-4,20-dioxo- 6,9,12,15,18-pentaoxa-3- azaicosanoyl)-4-hydroxy-N- (4-(4-methylthiazol-5- yl)benzyl)pyrrolidine-2- carboxamide |
| JA-084 | 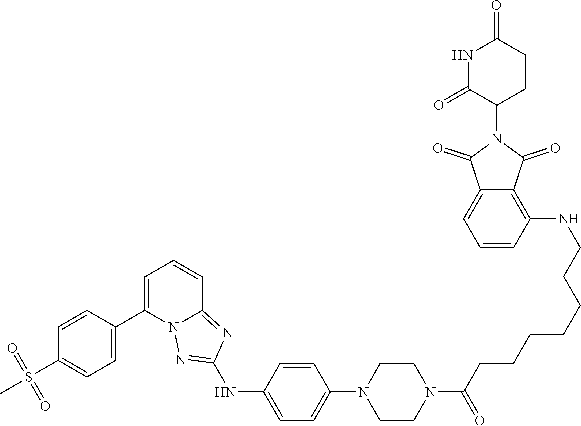 [SMILES: CS(=O)(=O)c1ccc(-c2cccc3nc(Nc4ccc(N5CCN(C(=O)CCCCCCCNc6cccc7c6C(=O)N(C6CCC(=O)NC6=O)C7=O)CC5)cc4)nn23)cc1] | 2-(2,6-Dioxo-piperidin-3-yl)- 4-[8-(4-{4-[5-(4- methanesulfonyl-phenyl)- [1,2,4]triazolo[1,5-a]pyridin- 2-ylamino]-phenyl}- piperazin-1-yl)-8-oxo- octylamino]-isoindole-1,3- dione |
| JA-085 | 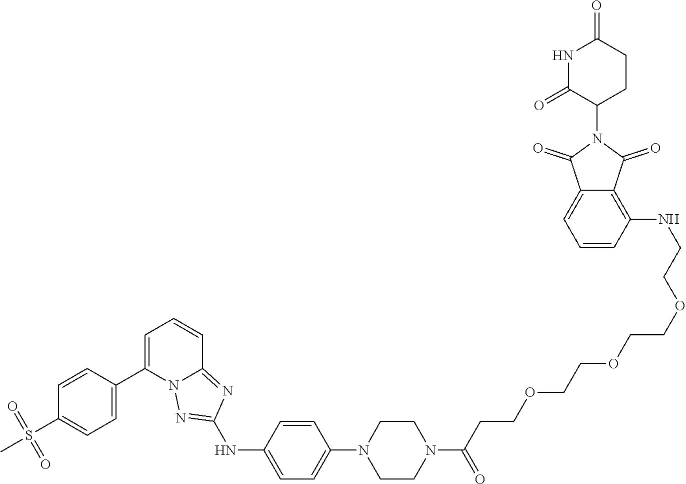 [SMILES: CS(=O)(=O)c1ccc(-c2cccc3nc(Nc4ccc(N5CCN(C(=O)CCOCCOCCOCCNc6cccc7c6C(=O)N(C6CCC(=O)NC6=O)C7=O)CC5)cc4)nn23)cc1] | 2-(2,6-Dioxo-piperidin-3-yl)- 4-[2-(2-{2-[3-(4-{4-[5-(4- methanesulfonyl-phenyl)- [1,2,4]triazolo[1,5-a]pyridin- 2-ylamino]-phenyl}- piperazin-1-yl)-3-oxo- propoxy]-ethoxy}-ethoxy)- ethylamino]-isoindole-1,3- dione |
| JA-086 | 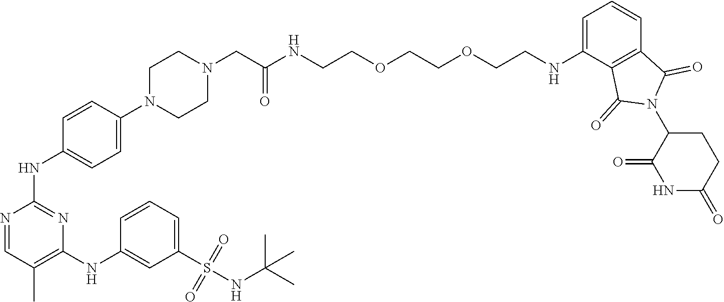 [SMILES: Cc1cnc(Nc2ccc(N3CCN(CC(=O)NCCOCCOCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-(2-(2-((2-(2,6- dioxopiperidin-3-yl)-1,3- dioxoisoindolin-4- yl)amino)ethoxy)ethoxy) ethyl)acetamide |
| JA-087 | 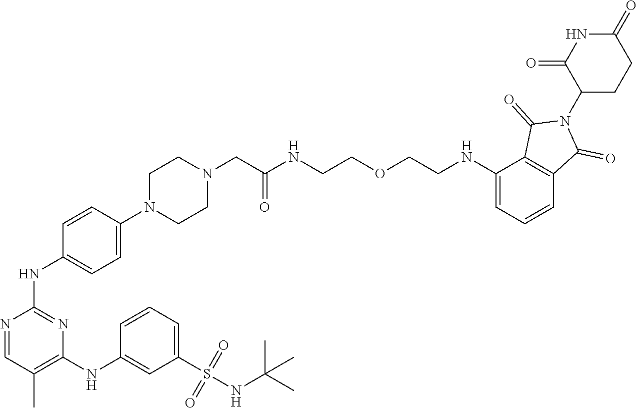 [SMILES: Cc1cnc(Nc2ccc(N3CCN(CC(=O)NCCOCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-(2-((2-(2,6- dioxopiperidin-3-yl)-1,3- dioxoisoindolin-4- yl)amino)ethoxy)ethyl) acetamide |
| JA-088 | 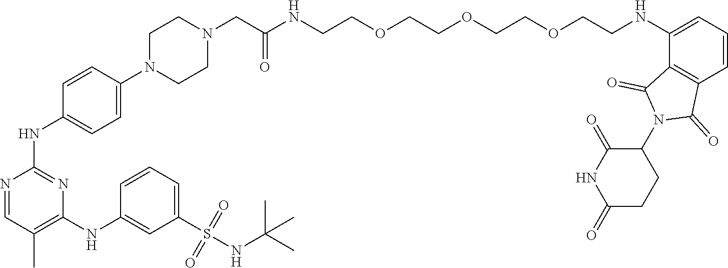 [SMILES: Cc1cnc(Nc2ccc(N3CCN(CC(=O)NCCOCCOCCOCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-(2-(2-(2-((2-(2,6- dioxopiperidin-3-yl)-1,3- dioxoisoindolin-4- yl)amino)ethoxy)ethoxy)ethoxy) ethyl)acetamide |
| JA-089 | 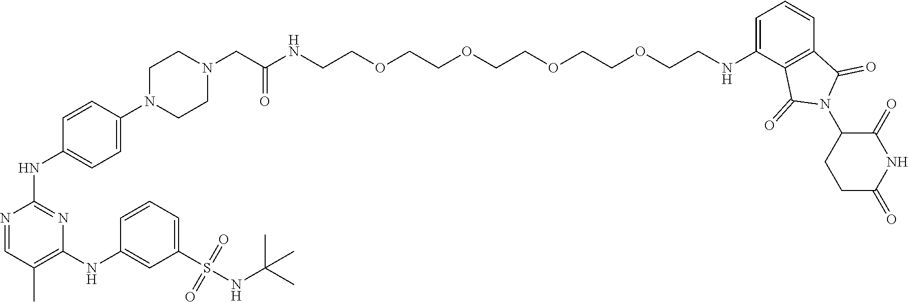 [SMILES: Cc1cnc(Nc2ccc(N3CCN(CC(=O)NCCOCCOCCOCCOCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(14-((2-(2,6- dioxopiperidin-3-yl)-1,3- dioxoisoindolin-4-yl)amino) 3,6,9,12- tetraoxatetradecyl)acetamide |
| JA-090 | 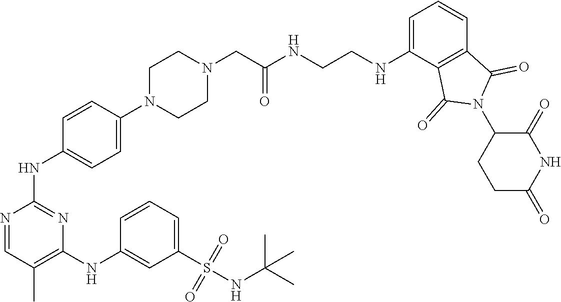 [SMILES: Cc1cnc(Nc2ccc(N3CCN(CC(=O)NCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-((2-(2,6- dioxopiperidin-3-yl)-1,3- dioxoisoindolin-4- yl)amino)ethyl)acetamide |
| JA-091 | 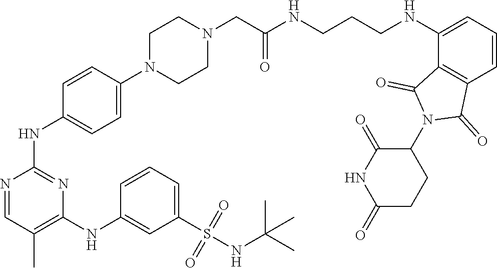 [SMILES: Cc1cnc(Nc2ccc(N3CCN(CC(=O)NCCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(3-((2-(2,6- dioxopiperidin-3-yl)-1,3- dioxoisoindolin-4- yl)amino)propyl)acetamide |
| JA-092 | 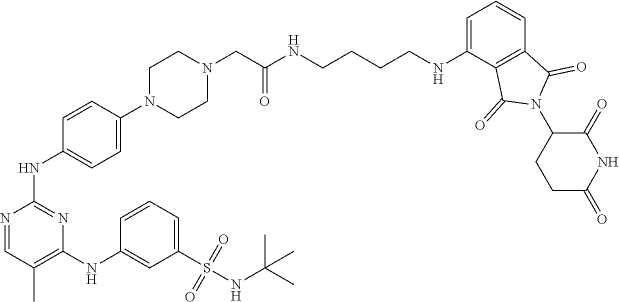 [SMILES: Cc1cnc(Nc2ccc(N3CCN(CC(=O)NCCCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(4-((2-(2,6- dioxopiperidin-3-yl)-1,3- dioxoisoindolin-4- yl)amino)butyl)acetamide |
| JA-093 | 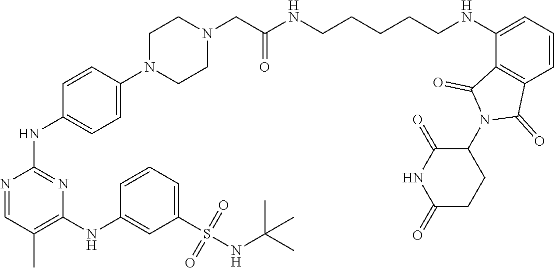 [SMILES: Cc1cnc(Nc2ccc(N3CCN(CC(=O)NCCCCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(5-((2-(2,6- dioxopiperidin-3-yl)-1,3- dioxoisoindolin-4- yl)amino)pentyl)acetamide |
| JA-094 | 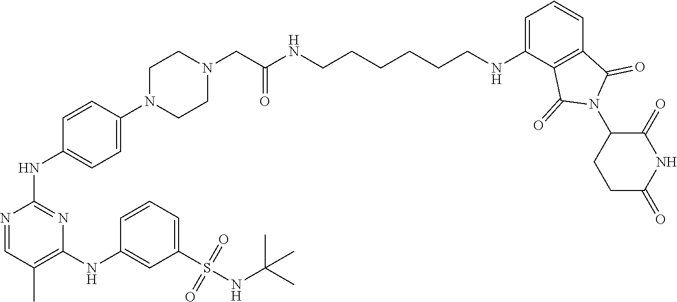 [SMILES: Cc1cnc(Nc2ccc(N3CCN(CC(=O)NCCCCCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(6-((2-(2,6- dioxopiperidin-3-yl)-1,3- dioxoisoindolin-4- yl)amino)hexyl)acetamide |
| JA-095 | 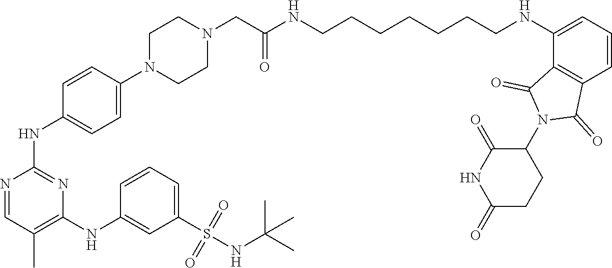 [SMILES: Cc1cnc(Nc2ccc(N3CCN(CC(=O)NCCCCCCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(7-((2-(2,6- dioxopiperidin-3-yl)-1,3- dioxoisoindolin-4- yl)amino)heptyl)acetamide |
| JA-096 | 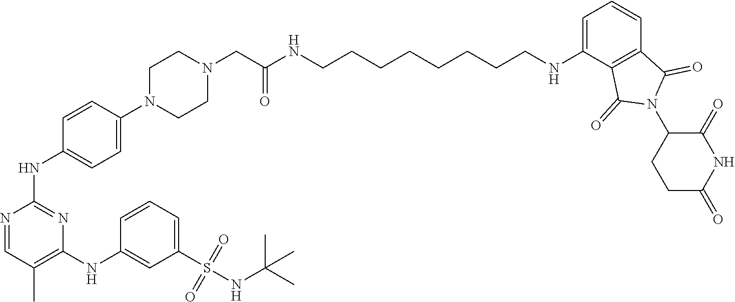 [SMILES: Cc1cnc(Nc2ccc(N3CCN(CC(=O)NCCCCCCCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(8-((2-(2,6- dioxopiperidin-3-yl)-1,3- dioxoisoindolin-4- yl)amino)octyl)acetamide |
| JA-097 | 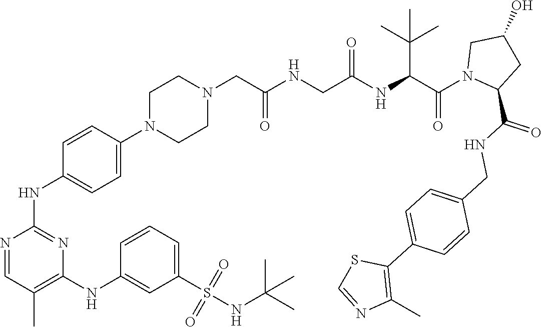 [SMILES: Cc1cnc(Nc2ccc(N3CCN(CC(=O)NCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(2-(2-(4-(4- ((4-((3-(N-(tert- butyl)sulfamoyl)phenyl)amino)- 5-methylpyrimidin-2- yl)amino)phenyl)piperazin-1- yl)acetamido)acetamido)-3,3- dimethylbutanoyl)-4- hydroxy-N-(4-(4- methylthiazol-5- yl)benzyl)pyrrolidine-2- carboxamide |
| JA-098 | 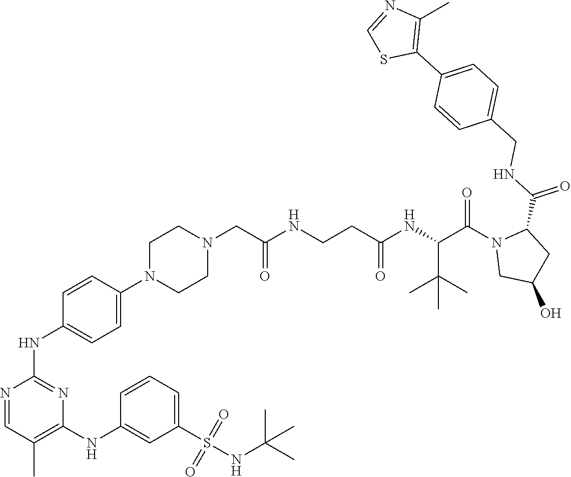 [SMILES: Cc1cnc(Nc2ccc(N3CCN(CC(=O)NCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(3-(2-(4-(4- ((4-((3-(N-(tert- butyl)sulfamoyl)phenyl)amino)- 5-methylpyrimidin-2- yl)amino)phenyl)piperazin-1- yl)acetamido)acetamido)- 3,3-dimethylbutanoyl)-4- hydroxy-N-(4-(4- methylthiazol-5- yl)benzyl)pyrrolidine-2- carboxamide |
| JA-099 | 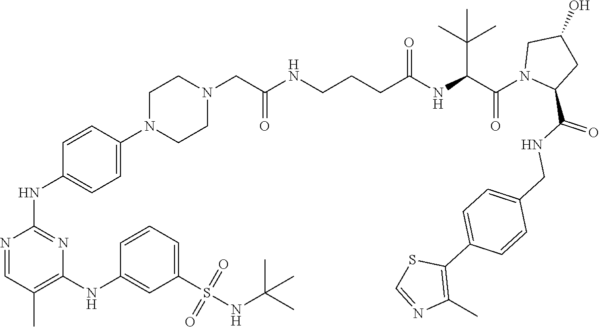 [SMILES: Cc1cnc(Nc2ccc(N3CCN(CC(=O)NCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(4-(2-(4-(4- ((4-((3-(N-(tert- butyl)sulfamoyl)phenyl)amino)- 5-methylpyrimidin-2- yl)amino)phenyl)piperazin-1- yl)acetamido)butanamido)- 3,3-dimethylbutanoyl)-4- hydroxy-N-(4-(4- methylthiazol-5- yl)benzyl)pyrrolidine-2- carboxamide |
| JA-100 | 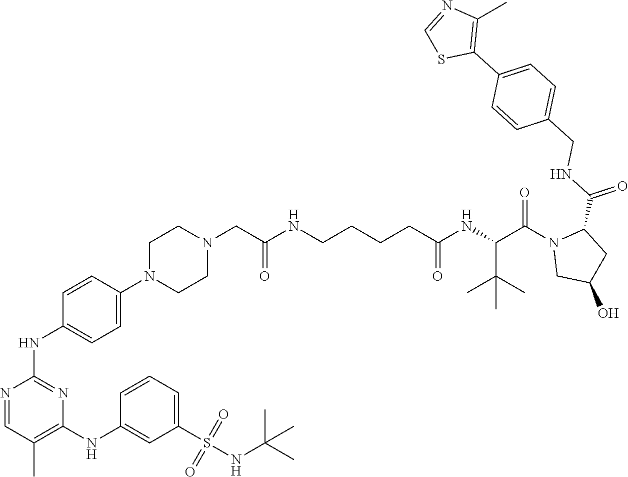 [SMILES: Cc1cnc(Nc2ccc(N3CCN(CC(=O)NCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(5-(2-(4-(4- ((4-((3-(N-(tert- butyl)sulfamoyl)phenyl)amino)- 5-methylpyrimidin-2- yl)amino)phenyl)piperazin-1- yl)acetamido)pentanamido)- 3,3-dimethylbutanoyl)-4- hydroxy-N-(4-(4- methylthiazol-5- yl)benzyl)pyrrolidine-2- carboxamide |
| JA-101 | 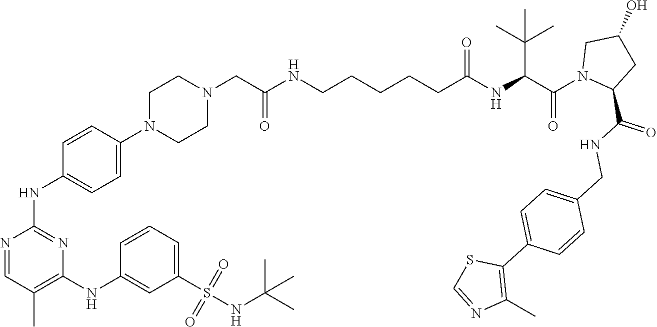 [SMILES: Cc1cnc(Nc2ccc(N3CCN(CC(=O)NCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(6-(2-(4-(4- ((4-((3-(N-(tert- butyl)sulfamoyl)phenyl)amino)- 5-methylpyrimidin-2- yl)amino)phenyl)piperazin-1- yl)acetamido)hexanamido)- 3,3-dimethylbutanoyl)-4- hydroxy-N-(4-(4- methylthiazol-5- yl)benzyl)pyrrolidine-2- carboxamide |
| JA-102 | 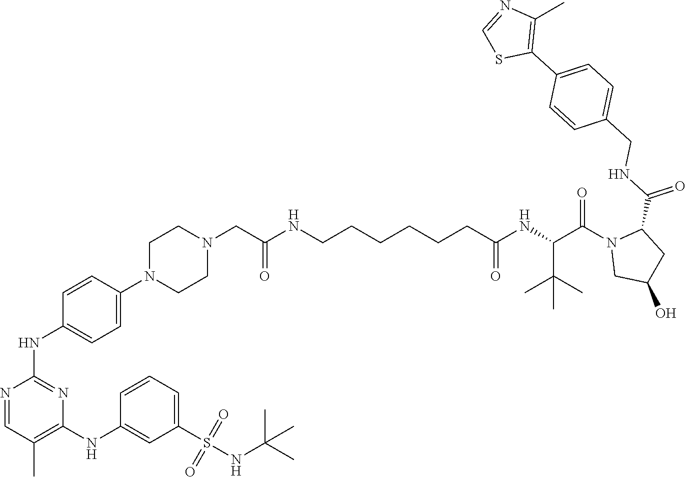 [SMILES: Cc1cnc(Nc2ccc(N3CCN(CC(=O)NCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(7-(2-(4-(4- ((4-((3-(N-(tert- butyl)sulfamoyl)phenyl)amino)- 5-methylpyrimidin-2- yl)amino)phenyl)piperazin-1- yl)acetamido)heptanamido)- 3,3-dimethylbutanoyl)-4- hydroxy-N-(4-(4- methylthiazol-5- yl)benzyl)pyrrolidine-2- carboxamide |
| JA-103 | 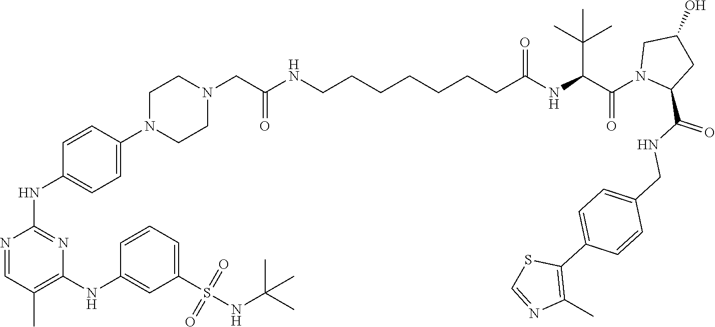 [SMILES: Cc1cnc(Nc2ccc(N3CCN(CC(=O)NCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(8-(2-(4-(4- ((4-((3-(N-(tert- butyl)sulfamoyl)phenyl)amino)- 5-methylpyrimidin-2- yl)amino)phenyl)piperazin-1- yl)acetamido)octanamido)- 3,3-dimethylbutanoyl)-4- hydroxy-N-(4-(4- methylthiazol-5- yl)benzyl)pyrrolidine-2- carboxamide |
| JA-104 | 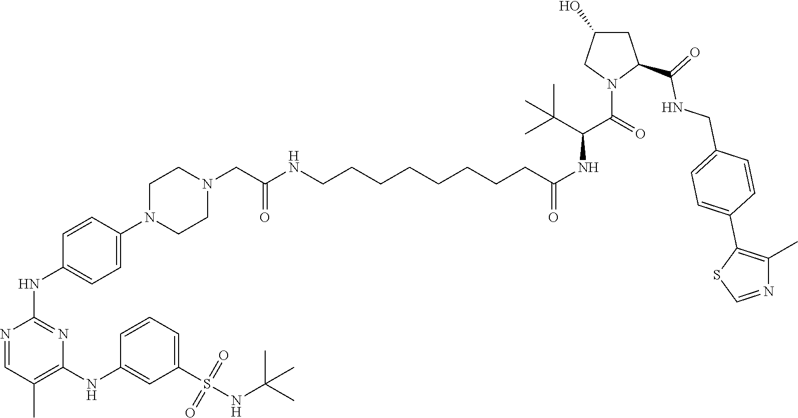 [SMILES: Cc1cnc(Nc2ccc(N3CCN(CC(=O)NCCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(9-(2-(4-(4- ((4-((3-(N-(tert- butyl)sulfamoyl)phenyl)amino)- 5-methylpyrimidin-2- yl)amino)phenyl)piperazin-1- yl)acetamido)nonanamido)- 3,3-dimethylbutanoyl)-4- hydroxy-N-(4-(4- methylthiazol-5- yl)benzyl)pyrrolidine-2- carboxamide |
| JA-105 | 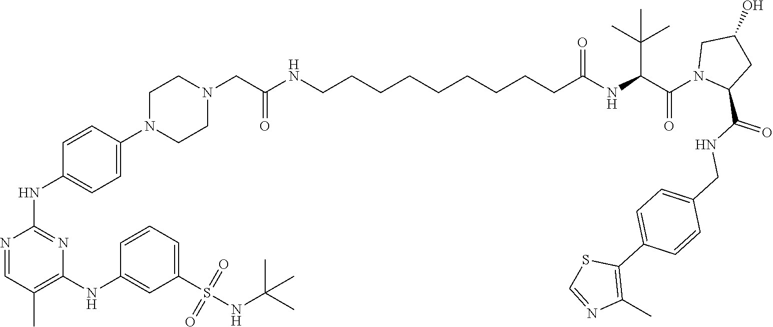 [SMILES: Cc1cnc(Nc2ccc(N3CCN(CC(=O)NCCCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(10-(2-(4- ((4-((3-(N-(tert- butyl)sulfamoyl)phenyl)amino)- 5-methylpyrimidin-2- yl)amino)phenyl)piperazin-1- yl)acetamido)decanamido)- 3,3-dimethylbutanoyl)-4- hydroxy-N-(4-(4- methylthiazol-5- yl)benzyl)pyrrolidine-2- carboxamide |
| JA-106 | 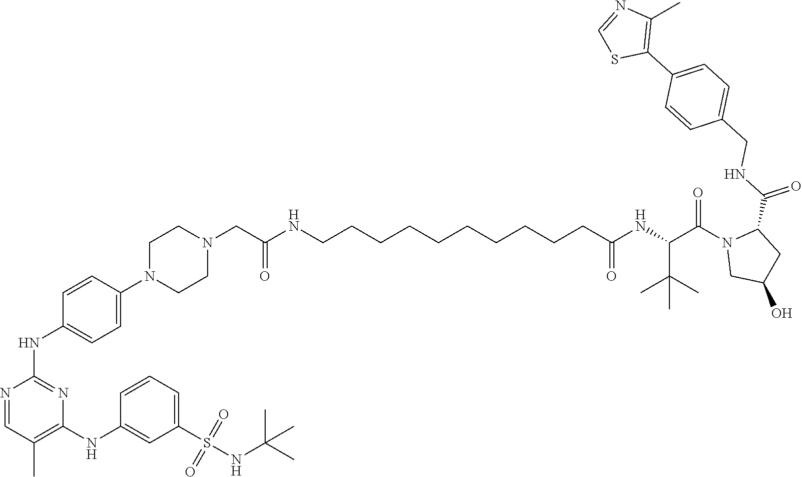 [SMILES: Cc1cnc(Nc2ccc(N3CCN(CC(=O)NCCCCCCCCCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(11-(2-(4- ((4-((3-(N-(tert- butyl)sulfamoyl)phenyl)amino)- 5-methylpyrimidin-2- yl)amino)phenyl)piperazin-1- yl)acetamido)undecanaamido)- 3,3-dimethylbutanoyl)-4- hydroxy-N-(4-(4- methylthiazol-5- yl)benzyl)pyrrolidine-2- carboxamide |
| JA-107 | 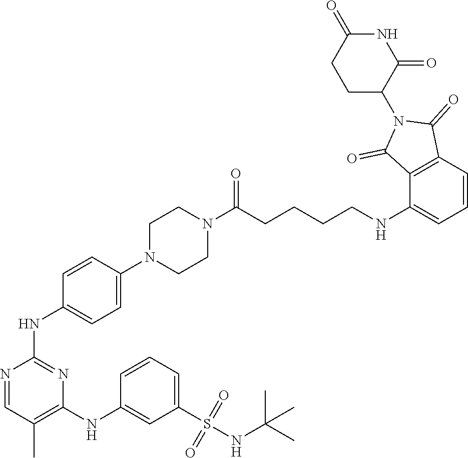 [SMILES: Cc1cnc(Nc2ccc(N3CCN(C(=O)CCCCNc4cccc5c4C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(5- ((2-(2,6-dioxopiperidin-3-yl)- 1,3-dioxoisoindolin-4- yl)amino)pentanoyl)piperazin- 1-yl)phenyl)amino)-5- methylpyrimidin-4- yl)amino)benzenesulfonamide |
| JA-108 | 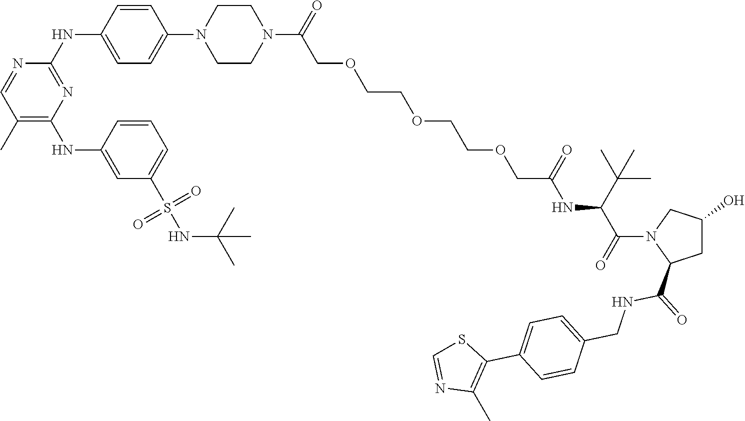 [SMILES: Cc1cnc(Nc2ccc(N3CCN(C(=O)COCCOCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 14-(4-(4-((4-((3-(N-(tert- butyl)sulfamoyl)phenyl)amino)- 5-methylpyrimidin-2- yl)amino)phenyl)piperazin-1- yl)-4,14-dioxo-6,9,12-trioxa- 3-azatetradecanoyl)-4- hydroxy-N-(4-(4- methylthiazol-5- yl)benzyl)pyrrolidine-2- carboxamide |
| JA-109 | 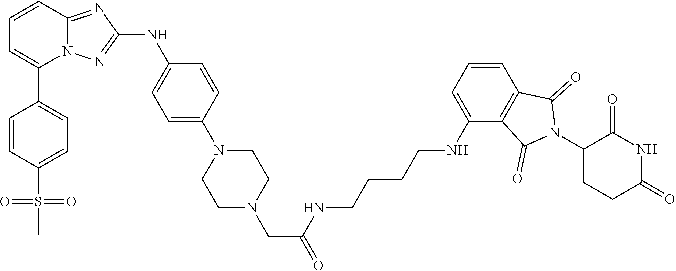 [SMILES: CS(=O)(=O)c1ccc(-c2cccc3nc(Nc4ccc(N5CCN(CC(=O)NCCCCNc6cccc7c6C(=O)N(C6CCC(=O)NC6=O)C7=O)CC5)cc4)nn23)cc1] | N-(4-((2-(2,6-dioxopiperidin- 3-yl)-1,3-dioxoisoindolin-4- yl)amino)butyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-110 | 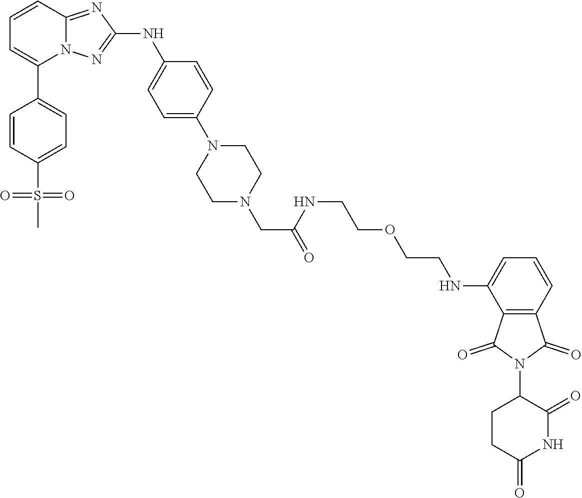 [SMILES: CS(=O)(=O)c1ccc(-c2cccc3nc(Nc4ccc(N5CCN(CC(=O)NCCOCCNc6cccc7c6C(=O)N(C6CCC(=O)NC6=O)C7=O)CC5)cc4)nn23)cc1] | N-(2-(2-((2-(2,6- dioxopiperidin-3-yl)-1,3- dioxoisoindolin-4- yl)amino)ethoxy)ethyl)-2-(4- (4-((5-(4- (methylsulfonyl)phenyl)- [1,2,4]triazolo[1,5-a]pyridin- 2-yl)amino)phenyl)piperazin- 1-yl)acetamide |
| JA-111 | 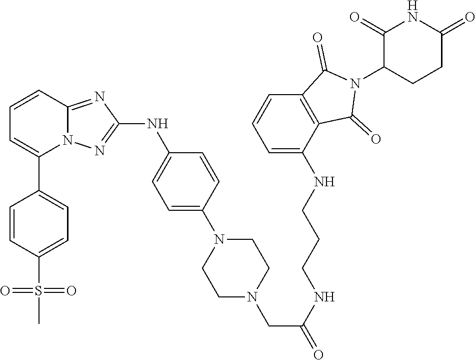 [SMILES: CS(=O)(=O)c1ccc(-c2cccc3nc(Nc4ccc(N5CCN(CC(=O)NCCCNc6cccc7c6C(=O)N(C6CCC(=O)NC6=O)C7=O)CC5)cc4)nn23)cc1] | N-(3-((2-(2,6-dioxopiperidin- 3-yl)-1,3-dioxoisoindolin-4- yl)amino)propyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-112 | 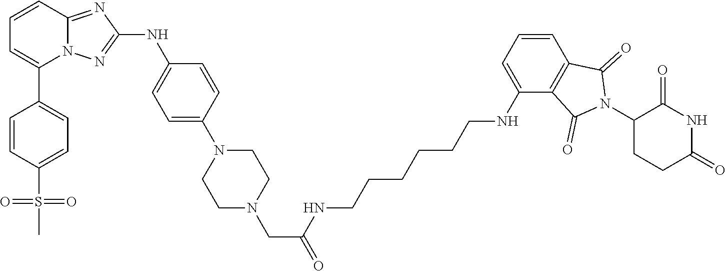 [SMILES: CS(=O)(=O)c1ccc(-c2cccc3nc(Nc4ccc(N5CCN(CC(=O)NCCCCCCNc6cccc7c6C(=O)N(C6CCC(=O)NC6=O)C7=O)CC5)cc4)nn23)cc1] | N-(6-((2-(2,6-dioxopiperidin- 3-yl)-1,3-dioxoisoindolin-4- yl)amino)hexyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-113 | 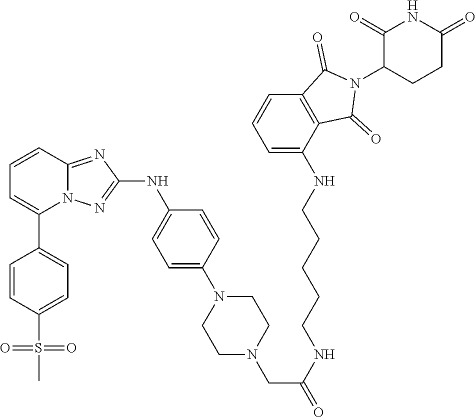 [SMILES: CS(=O)(=O)c1ccc(-c2cccc3nc(Nc4ccc(N5CCN(CC(=O)NCCCCCNc6cccc7c6C(=O)N(C6CCC(=O)NC6=O)C7=O)CC5)cc4)nn23)cc1] | N-(5-((2-(2,6-dioxopiperidin- 3-yl)-1,3-dioxoisoindolin-4- yl)amino)pentyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-114 | 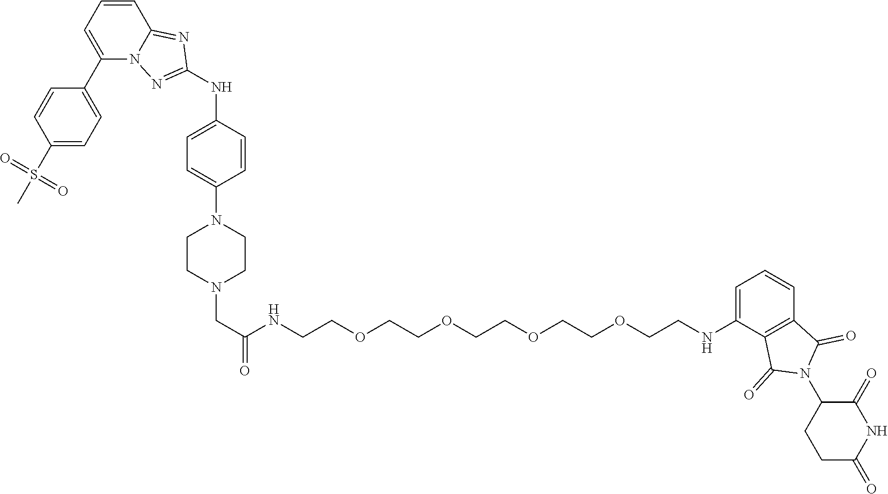 [SMILES: CS(=O)(=O)c1ccc(-c2cccc3nc(Nc4ccc(N5CCN(CC(=O)NCCOCCOCCOCCOCCNc6cccc7c6C(=O)N(C6CCC(=O)NC6=O)C7=O)CC5)cc4)nn23)cc1] | N-(14-((2-(2,6- dioxopiperidin-3-yl)-1,3- dioxoisoindolin-4-yl)amino)- 3,6,9,12-tetraoxatetradecyl)- 2-(4-(4-((5-(4- (methylsulfonyl)phenyl)- [1,2,4]triazolo[1,5-a]pyridin- 2-yl)amino)phenyl)piperazin- 1-yl)acetamide |
| JA-115 | 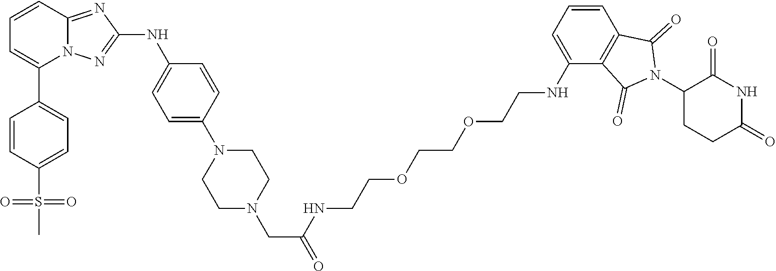 [SMILES: CS(=O)(=O)c1ccc(-c2cccc3nc(Nc4ccc(N5CCN(CC(=O)NCCOCCOCCNc6cccc7c6C(=O)N(C6CCC(=O)NC6=O)C7=O)CC5)cc4)nn23)cc1] | N-(2-(2-(2-((2-(2,6- dioxopiperidin-3-yl)-1,3- dioxoisoindolin-4- yl)amino)ethoxy)ethoxy)ethyl)- 2-(4-(4-((5-(4- (methylsulfonyl)phenyl)- [1,2,4]triazolo[1,5-a]pyridin- 2-yl)amino)phenyl)piperazin- 1-yl)acetamide |
| JA-116 | 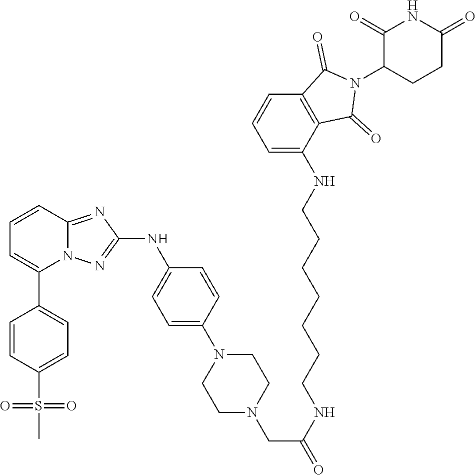 [SMILES: CS(=O)(=O)c1ccc(-c2cccc3nc(Nc4ccc(N5CCN(CC(=O)NCCCCCCCNc6cccc7c6C(=O)N(C6CCC(=O)NC6=O)C7=O)CC5)cc4)nn23)cc1] | N-(7-((2-(2,6-dioxopiperidin- 3-yl)-1,3-dioxoisoindolin-4- yl)amino)heptyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-117 | 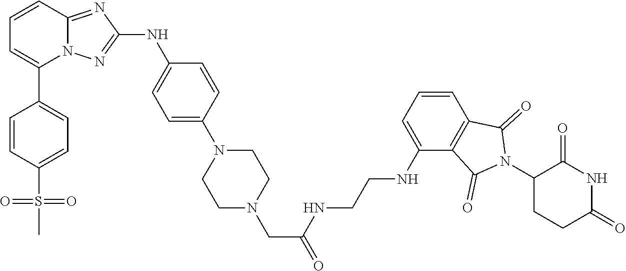 [SMILES: CS(=O)(=O)c1ccc(-c2cccc3nc(Nc4ccc(N5CCN(CC(=O)NCCNc6cccc7c6C(=O)N(C6CCC(=O)NC6=O)C7=O)CC5)cc4)nn23)cc1] | N-(2-((2-(2,6-dioxopiperidin- 3-yl)-1,3-dioxoisoindolin-4- yl)amino)ethyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-118 | 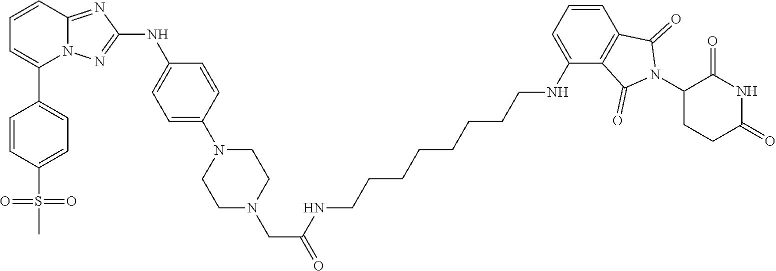 [SMILES: CS(=O)(=O)c1ccc(-c2cccc3nc(Nc4ccc(N5CCN(CC(=O)NCCCCCCCCNc6cccc7c6C(=O)N(C6CCC(=O)NC6=O)C7=O)CC5)cc4)nn23)cc1] | N-(8-((2-(2,6-dioxopiperidin- 3-yl)-1,3-dioxoisoindolin-4- yl)amino)octyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-119 | 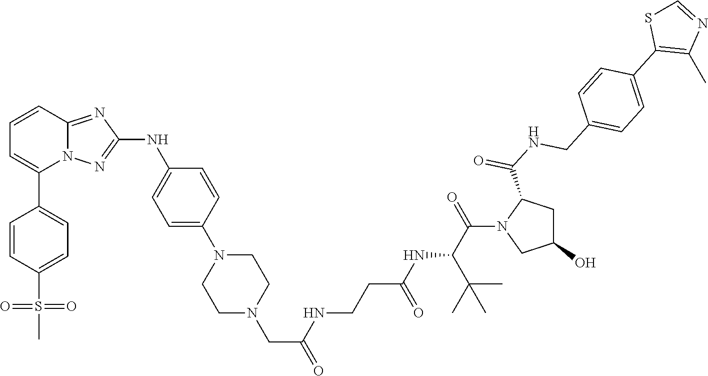 [SMILES: Cc1ncsc1-c1ccc(CNC(=O)[C@@H]2C[C@@H](O)CN2C(=O)[C@@H](NC(=O)CCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(3-(2-(4-(4-((5-(4- (methylsulfonyl)phenyl)- [1,2,4]triazolo[1,5-a]pyridin- 2-yl)amino)phenyl)piperazin- 1- yl)acetamido)propanamido) butanoyl)-4-hydroxy-N-(4-(4- methylthiazol-5- yl)benzyl)pyrrolidine-2- carboxamide |
| JA-120 | 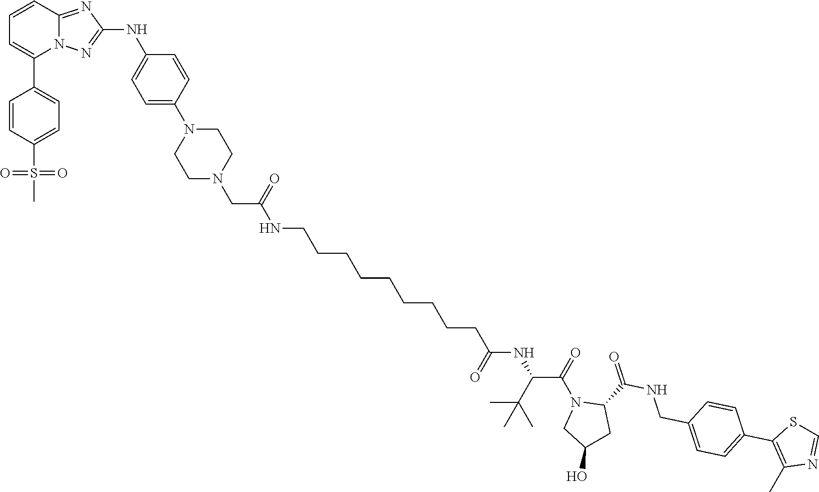 [SMILES: Cc1ncsc1-c1ccc(CNC(=O)[C@@H]2C[C@@H](O)CN2C(=O)[C@@H](NC(=O)CCCCCCCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(10-(2-(4-(4-((5-(4- (methylsulfonyl)phenyl)- [1,2,4]triazolo[1,5-a]pyridin- 2-yl)amino)phenyl)piperazin- 1- yl)acetamido)decanamido) butanoyl)-4-hydroxy-N-(4-(4- methylthiazol-5- yl)benzyl)pyrrolidine-2- carboxamide |
| JA-121 | 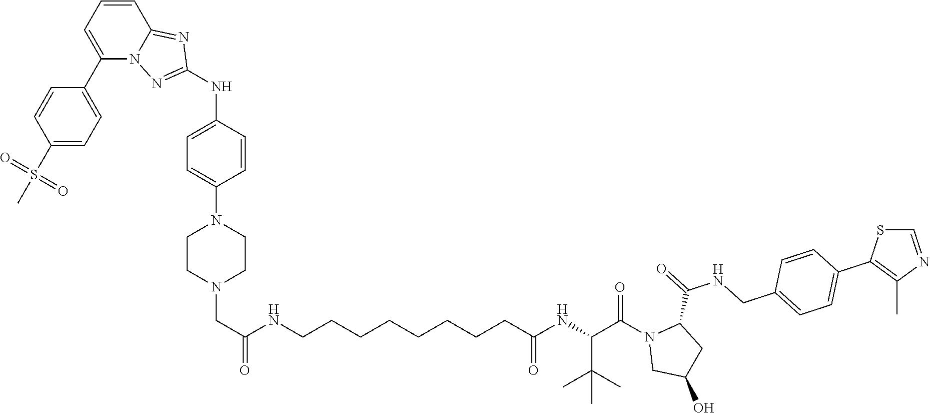 [SMILES: Cc1ncsc1-c1ccc(CNC(=O)[C@@H]2C[C@@H](O)CN2C(=O)[C@@H](NC(=O)CCCCCCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(9-(2-(4-(4-((5-(4- (methylsulfonyl)phenyl)- [1,2,4]triazolo[1,5-a]pyridin- 2-yl)amino)phenyl)piperazin- 1- yl)acetamido)nonanamido) butanoyl)-4-hydroxy-N-(4-(4- methylthiazol-5- yl)benzyl)pyrrolidine-2- carboxamide |
| JA-122 | 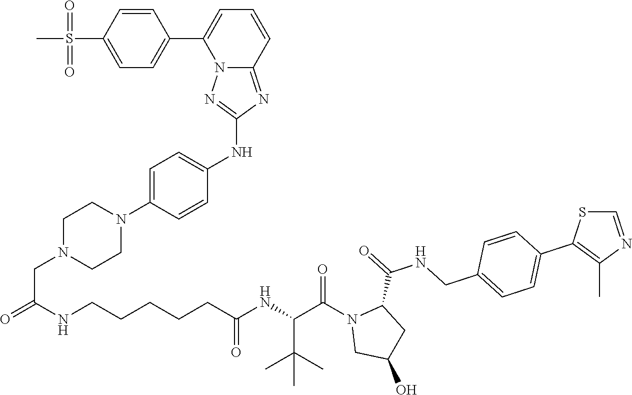 [SMILES: Cc1ncsc1-c1ccc(CNC(=O)[C@@H]2C[C@@H](O)CN2C(=O)[C@@H](NC(=O)CCCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(6-(2-(4-(4-((5-(4- (methylsulfonyl)phenyl)- [1,2,4]triazolo[1,5-a]pyridin- 2-yl)amino)phenyl)piperazin- 1- yl)acetamido)hexanamido) butanoyl)-4-hydroxy-N-(4-(4- methylthiazol-5- yl)benzyl)pyrrolidine-2- carboxamide |
| JA-123 | 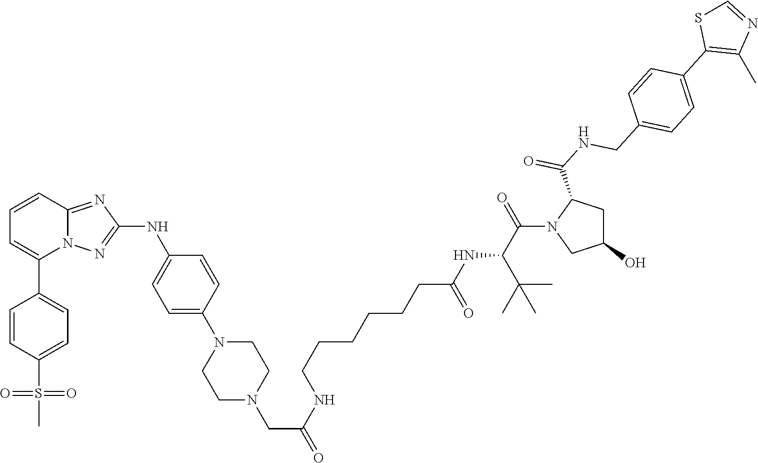 [SMILES: Cc1ncsc1-c1ccc(CNC(=O)[C@@H]2C[C@@H](O)CN2C(=O)[C@@H](NC(=O)CCCCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(7-(2-(4-(4-((5-(4- (methylsulfonyl)phenyl)- [1,2,4]triazolo[1,5-a]pyridin- 2-yl)amino)phenyl)piperazin- 1- yl)acetamido)heptanamido) butanoyl)-4-hydroxy-N-(4-(4- methylthiazol-5- yl)benzyl)pyrrolidine-2- carboxamide |
| JA-124 | 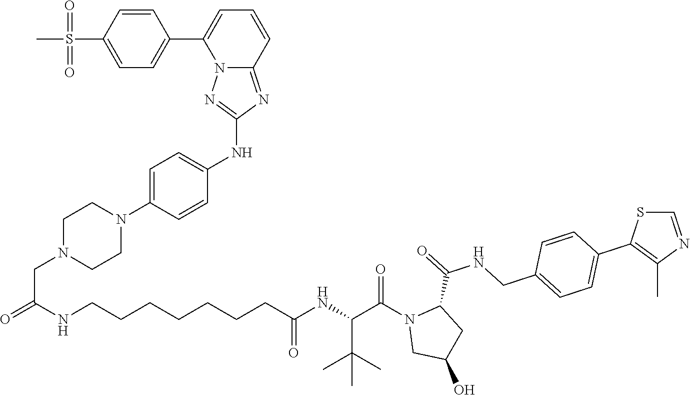 [SMILES: Cc1ncsc1-c1ccc(CNC(=O)[C@@H]2C[C@@H](O)CN2C(=O)[C@@H](NC(=O)CCCCCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(8-(2-(4-(4-((5-(4- (methylsulfonyl)phenyl)- [1,2,4]triazolo[1,5-a]pyridin- 2-yl)amino)phenyl)piperazin- 1- yl)acetamido)octanamido) butanoyl)-4-hydroxy-N-(4-(4- methylthiazol-5- yl)benzyl)pyrrolidine-2- carboxamide |
| JA-125 | 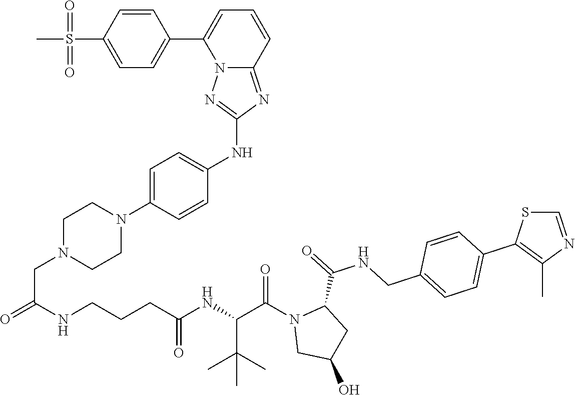 [SMILES: Cc1ncsc1-c1ccc(CNC(=O)[C@@H]2C[C@@H](O)CN2C(=O)[C@@H](NC(=O)CCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(4-(2-(4-(4-((5-(4- (methylsulfonyl)phenyl)- [1,2,4]triazolo[1,5-a]pyridin- 2-yl)amino)phenyl)piperazin- 1- yl)acetamido)butanamido) butanoyl)-4-hydroxy-N-(4-(4- methylthiazol-5- yl)benzyl)pyrrolidine-2- carboxamide |
| JA-126 | 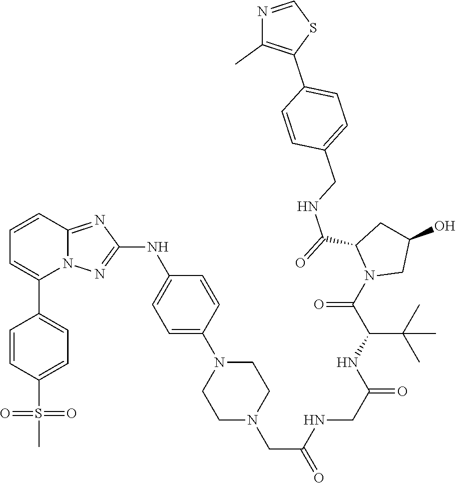 [SMILES: Cc1ncsc1-c1ccc(CNC(=O)[C@@H]2C[C@@H](O)CN2C(=O)[C@@H](NC(=O)CNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(2-(2-(4-(4-((5-(4- (methylsulfonyl)phenyl)- [1,2,4]triazolo[1,5-a]pyridin- 2-yl)amino)phenyl)piperazin- 1- yl)acetamido)acetamido) butanoyl)-4-hydroxy-N-(4-(4- methylthiazol-5- yl)benzyl)pyrrolidine-2- carboxamide |
| JA-127 | 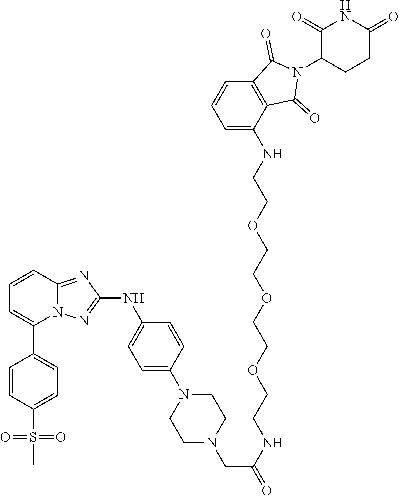 [SMILES: CS(=O)(=O)c1ccc(-c2cccc3nc(Nc4ccc(N5CCN(CC(=O)NCCOCCOCCOCCNc6cccc7c6C(=O)N(C6CCC(=O)NC6=O)C7=O)CC5)cc4)nn23)cc1] | N-(2-(2-(2-(2-((2-(2,6- dioxopiperidin-3-yl)-1,3- dioxoisoindolin-4- yl)amino)ethoxy)ethoxy)ethoxy) ethyl)-2-(4-(4-((5-(4- (methylsulfonyl)phenyl)- [1,2,4]triazolo[1,5-a]pyridin- 2-yl)amino)phenyl)piperazin- 1-yl)acetamide |
| JA-128 | 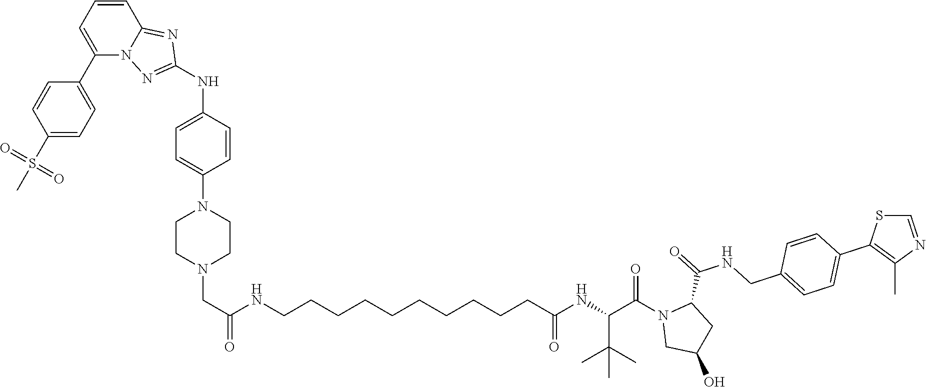 [SMILES: Cc1ncsc1-c1ccc(CNC(=O)[C@@H]2C[C@@H](O)CN2C(=O)[C@@H](NC(=O)CCCCCCCCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(11-(2-(4-(4-((5-(4- (methylsulfonyl)phenyl)- [1,2,4]triazolo[1,5-a]pyridin- 2-yl)amino)phenyl)piperazin- 1- yl)acetamido)undecanamido) butanoyl)-4-hydroxy-N-(4- (4-methylthiazol-5- yl)benzyl)pyrrolidine-2- carboxamide |
| JA-129 | 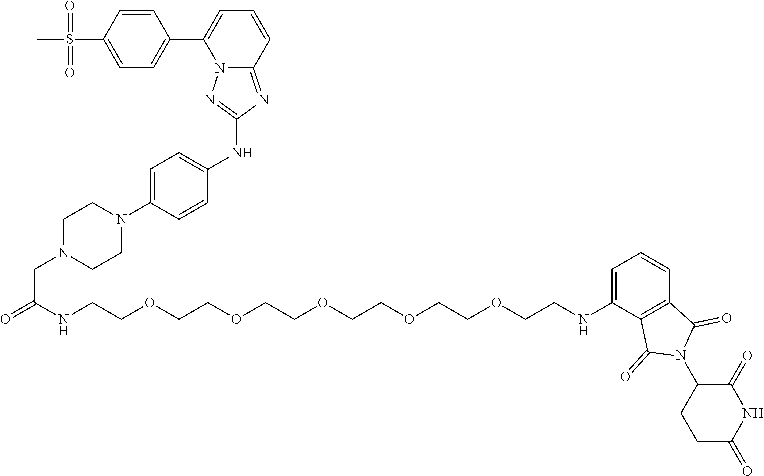 [SMILES: CS(=O)(=O)c1ccc(-c2cccc3nc(Nc4ccc(N5CCN(CC(=O)NCCOCCOCCOCCOCCOCCNc6cccc7c6C(=O)N(C6CCC(=O)NC6=O)C7=O)CC5)cc4)nn23)cc1] | N-(17-((2-(2,6- dioxopiperidin-3-yl)-1,3- dioxoisoindolin-4-yl)amino)- 3,6,12,15- pentaoxaheptadecyl)-2-(4-(4- ((5-(4- (methylsulfonyl)phenyl)- [1,2,4]triazolo[1,5-a]pyridin- 2-yl)amino)phenyl)piperazin- 1-yl)acetamide |
| JA-130 | 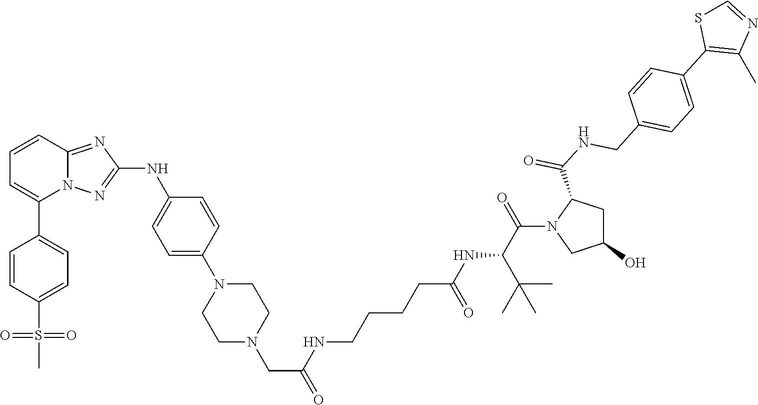 [SMILES: Cc1ncsc1-c1ccc(CNC(=O)[C@@H]2C[C@@H](O)CN2C(=O)[C@@H](NC(=O)CCCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(5-(2-(4-(4-((5-(4- (methylsulfonyl)phenyl)- [1,2,4]triazolo[1,5-a]pyridin- 2-yl)amino)phenyl)piperazin- 1- yl)acetamido)pentanamido) butanoyl)-4-hydroxy-N-(4-(4- methylthiazol-5- yl)benzyl)pyrrolidine-2- carboxamide |
| JA-131 | 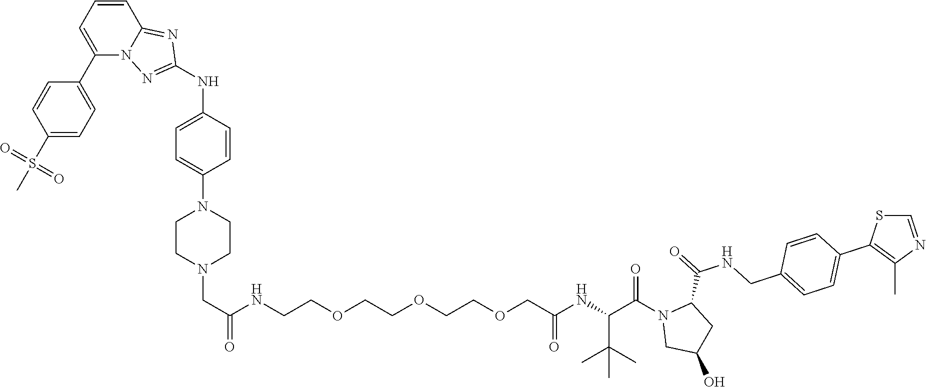 [SMILES: Cc1ncsc1-c1ccc(CNC(=O)[C@@H]2C[C@@H](O)CN2C(=O)[C@@H](NC(=O)COCCOCCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 17-(4-(4-((5-(4- (methylsulfonyl)phenyl)- [1,2,4]triazolo[1,5-a]pyridin- 2-yl)amino)phenyl)piperazin- 1-yl)-4,16-dioxo-6,9,12- trioxa-3,15- diazaheptadecanoyl)-4- hydroxy-N-(4-(4- methylthiazol-5- yl)benzyl)pyrrolidine-2- carboxamide |
| JA-132 | 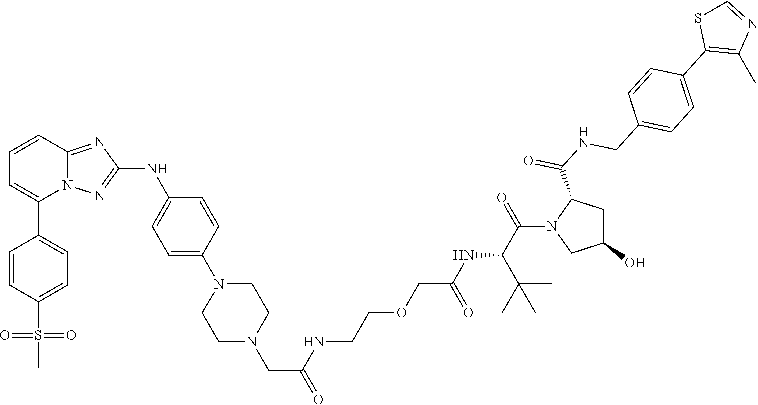 [SMILES: Cc1ncsc1-c1ccc(CNC(=O)[C@@H]2C[C@@H](O)CN2C(=O)[C@@H](NC(=O)COCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(2-(2-(2-(4-(4-((5-(4- (methylsulfonyl)phenyl)- [1,2,4]triazolo[1,5-a]pyridin- 2-yl)amino)phenyl)piperazin- 1- yl)acetamido)ethoxy)acetamido) butanoyl)-4-hydroxy-N- (4-(4-methylthiazol-5- yl)benzyl)pyrrolidine-2- carboxamide |
| JA-133 | 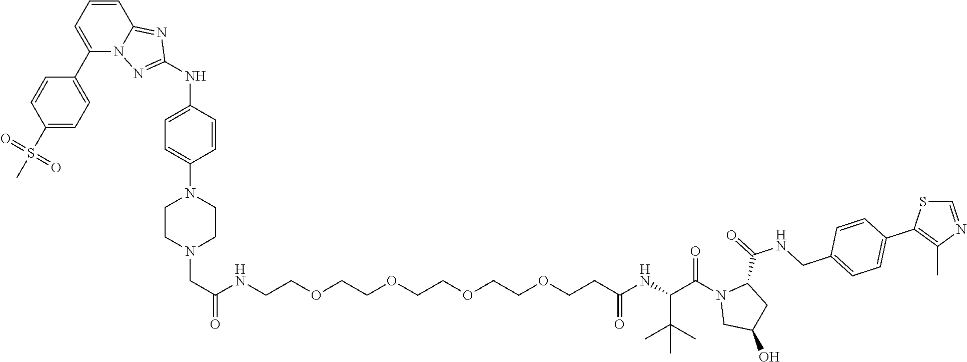 [SMILES: Cc1ncsc1-c1ccc(CNC(=O)[C@@H]2C[C@@H](O)CN2C(=O)[C@@H](NC(=O)CCOCCOCCOCCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-20-(tert- butyl)-1-(4-(4-((5-(4- (methylsulfonyl)phenyl)- [1,2,4]triazolo[1,5-a]pyridin- 2-yl)amino)phenyl)piperazin- 1-yl)-2,18-dioxo-6,9,12,15- tetraoxa-3,19- diazahenicosan-21-oyl)-4- hydroxy-N-(4-(4- methylthiazol-5- yl)benzyl)pyrrolidine-2- carboxamide |
| JA-134 | 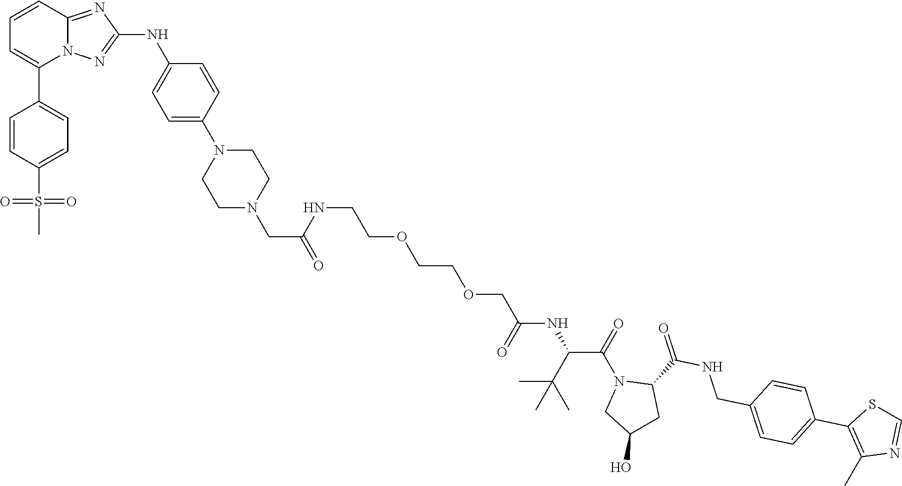 [SMILES: Cc1ncsc1-c1ccc(CNC(=O)[C@@H]2C[C@@H](O)CN2C(=O)[C@@H](NC(=O)COCCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-2-(tert-butyl)- 14-(4-(4-((5-(4- (methylsulfonyl)phenyl)- [1,2,4]triazolo[1,5-a]pyridin- 2-yl)amino)phenyl)piperazin- 1-yl)-4,13-dioxo-6,9-dioxa- 3,12-diazatetradecanoyl)-4- hydroxy-N-(4-(4- methylthiazol-5- yl)benzyl)pyrrolidine-2- carboxamide |
| JA-135 | 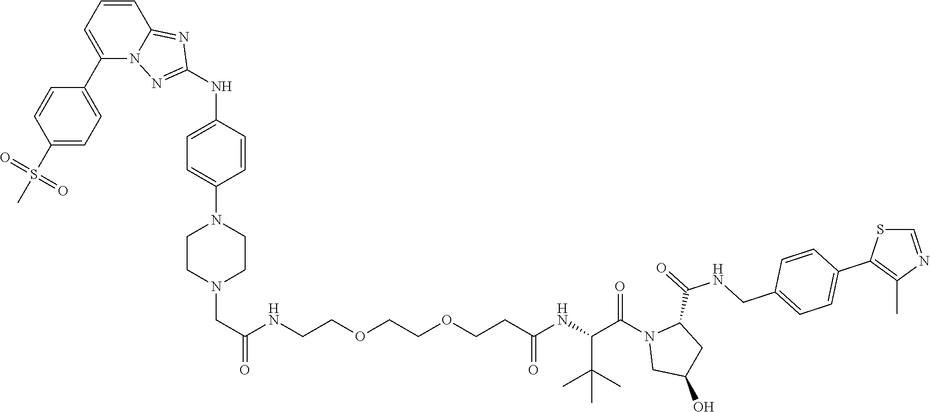 [SMILES: Cc1ncsc1-c1ccc(CNC(=O)[C@@H]2C[C@@H](O)CN2C(=O)[C@@H](NC(=O)CCOCCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-14-(tert- butyl)-1-(4-(4-((5-(4- (methylsulfonyl)phenyl)- [1,2,4]triazolo[1,5-a]pyridin- 2-yl)amino)phenyl)piperazin- 1-yl)-2,12-dioxo-6,9-dioxa- 3,13-diazapentadecan-15- oyl)-4-hydroxy-N-(4-(4- methylthiazol-5- yl)benzyl)pyrrolidine-2- carboxamide |
| JA-136 | 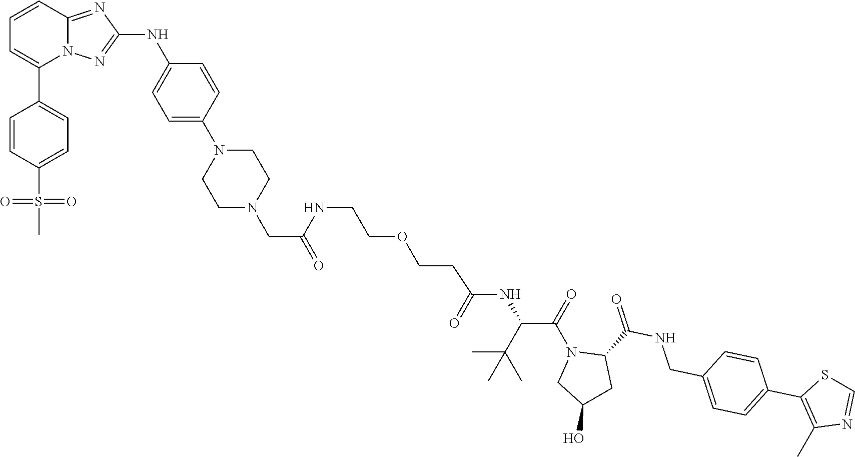 [SMILES: Cc1ncsc1-c1ccc(CNC(=O)[C@@H]2C[C@@H](O)CN2C(=O)[C@@H](NC(=O)CCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-3,3-dimethyl- 2-(3-(2-(2-(4-(4-((5-(4- (methylsulfonyl)phenyl)- [1,2,4]triazolo[1,5-a]pyridin- 2-yl)amino)phenyl)piperazin- 1- yl)acetamido)ethoxy) propanamido) butanoyl)-4-hydroxy- N-(4-(4-methylthiazol-5- yl)benzyl)pyrrolidine-2- carboxamide |
| JA-137 | 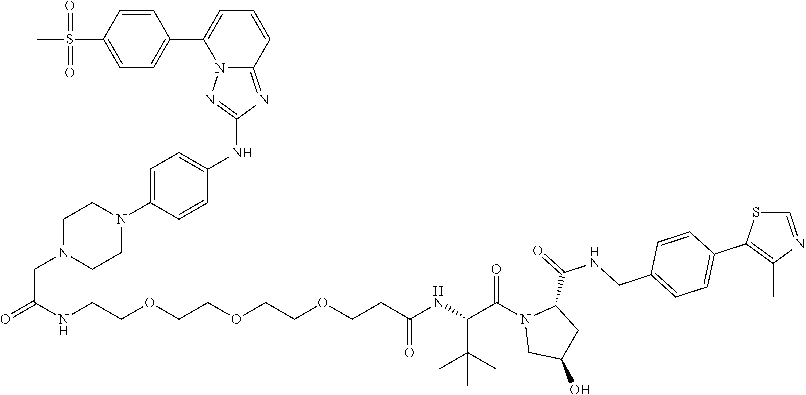 [SMILES: Cc1ncsc1-c1ccc(CNC(=O)[C@@H]2C[C@@H](O)CN2C(=O)[C@@H](NC(=O)CCOCCOCCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-17-(tert- butyl)-1-(4-(4-((5-(4- (methylsulfonyl)phenyl)- [1,2,4]triazolo[1,5-a]pyridin- 2-yl)amino)phenyl)piperazin- 1-yl)-2,15-dioxo-6,9,12- trioxa-3,16-diazapentadecan- 18-oyl)-4-hydroxy-N-(4-(4- methylthiazol-5- yl)benzyl)pyrrolidine-2- carboxamide |
| JA-138 | 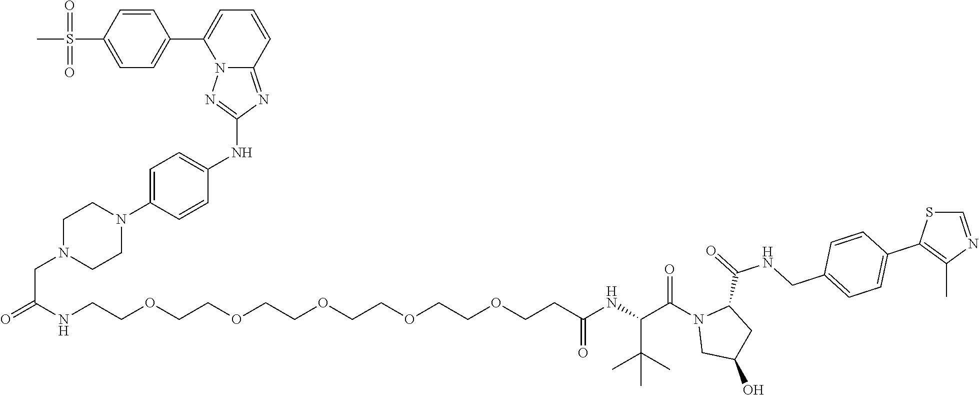 [SMILES: Cc1ncsc1-c1ccc(CNC(=O)[C@@H]2C[C@@H](O)CN2C(=O)[C@@H](NC(=O)CCOCCOCCOCCOCCOCCNC(=O)CN2CCN(c3ccc(Nc4nc5cccc(-c6ccc(S(C)(=O)=O)cc6)n5n4)cc3)CC2)C(C)(C)C)cc1] | (2S,4R)-1-((S)-23-(tert- butyl)-1-(4-(4-((5-(4- (methylsulfonyl)phenyl)- [1,2,4]triazolo[1,5-a]pyridin- 2-yl)amino)phenyl)piperazin- 1-yl)-2,21-dioxo- 6,9,12,15,18-pentaoxa-3,22- diazatetracosan-24-oyl)-4- hydroxy-N-(4-(4- methylthiazol-5- yl)benzyl)pyrrolidine-2- carboxamide |
| JA-139 | 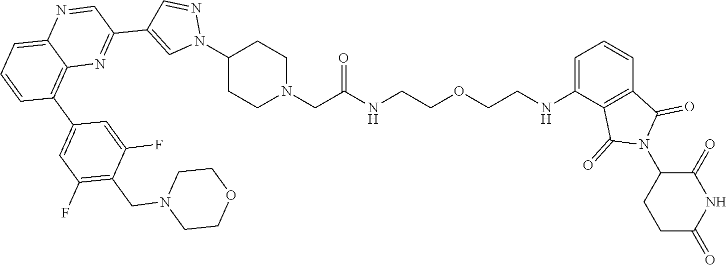 [SMILES: O=C(CN1CCC(n2cc(-c3cnc4cccc(-c5cc(F)c(CN6CCOCC6)c(F)c5)c4n3)cn2)CC1)NCCOCCNc1cccc2c1C(=O)N(C1CCC(=O)NC1=O)C2=O] | 2-[4-[4-[8-[3,5-difluoro-4- (morpholinomethyl)phenyl] quinoxalin-2-yl]pyrazol-1-yl]- 1-piperidyl]-N-[2-[2-[[2-(2,6- dioxo-3-piperidyl)-1,3-dioxo- isoindolin-4- yl]amino]ethoxy]ethyl]acetamide |
| JA-140 | 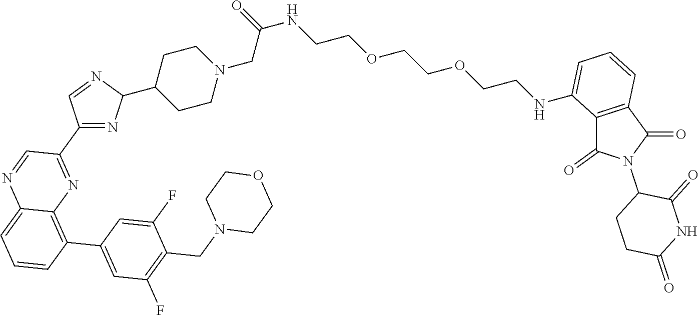 [SMILES: O=C(CN1CCC(C2N=CC(c3cnc4cccc(-c5cc(F)c(CN6CCOCC6)c(F)c5)c4n3)=N2)CC1)NCCOCCOCCNc1cccc2c1C(=O)N(C1CCC(=O)NC1=O)C2=O] | 2-[4-[4-[8-[3,5-difluoro-4- (morpholinomethyl)phenyl] quinoxalin-2-yl]pyrazol-1-yl]- 1-piperidyl]-N-[2-[2-[[2- (2,6-dioxo-3-piperidyl)-1,3- dioxo-isoindolin-4- yl]amino]ethoxy]ethoxy] ethyl]acetamide |
| JA-141 | 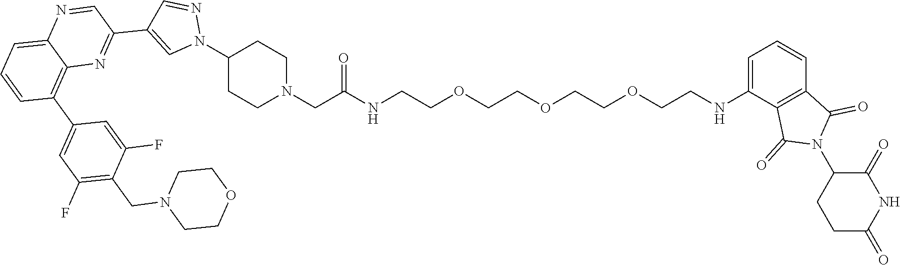 [SMILES: O=C(CN1CCC(n2cc(-c3cnc4cccc(-c5cc(F)c(CN6CCOCC6)c(F)c5)c4n3)cn2)CC1)NCCOCCOCCOCCNc1cccc2c1C(=O)N(C1CCC(=O)NC1=O)C2=O] | 2-[4-[4-[8-[3,5-difluoro-4- (morpholinomethyl)phenyl] quinoxalin-2-yl]pyrazol-1-yl]- 1-piperidyl]-N-[2-[2-[2-[2- [[2-(2,6-dioxo-3-piperidyl)- 1,3-dioxo-isoindolin-4- yl]amino]ethoxy]ethoxy]ethoxy] ethyl]acetamide |
| JA-142 | 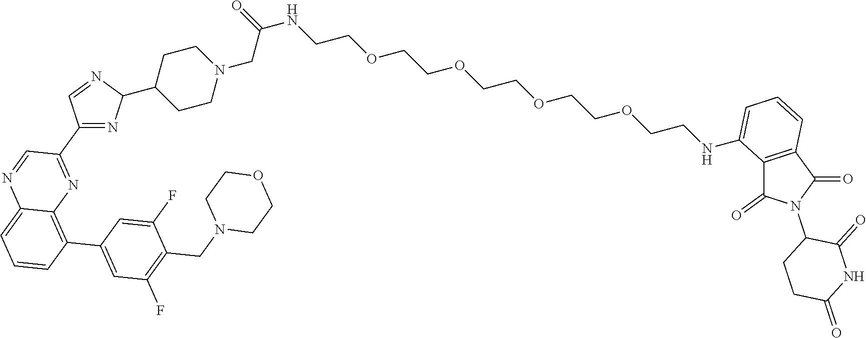 [SMILES: O=C(CN1CCC(C2N=CC(c3cnc4cccc(-c5cc(F)c(CN6CCOCC6)c(F)c5)c4n3)=N2)CC1)NCCOCCOCCOCCOCCNc1cccc2c1C(=O)N(C1CCC(=O)NC1=O)C2=O] | 2-[4-[4-[8-[3,5-difluoro-4- (morpholinomethyl)phenyl] quinoxalin-2-yl]pyrazol-1-yl]- 1-piperidyl]-N-[2-[2-[2-[2-[2- [[2-(2,6-dioxo-3-piperidyl)- 1,3-dioxo-isoindolin-4- yl]amino]ethoxy]ethoxy]ethoxy] ethoxy]ethyl]acetamide |
| JA-143 | 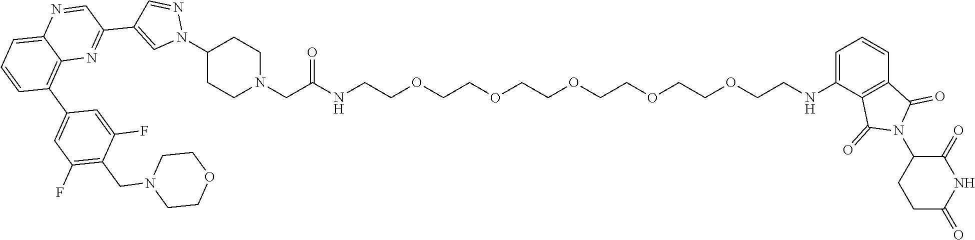 [SMILES: O=C(CN1CCC(n2cc(-c3cnc4cccc(-c5cc(F)c(CN6CCOCC6)c(F)c5)c4n3)cn2)CC1)NCCOCCOCCOCCOCCOCCNc1cccc2c1C(=O)N(C1CCC(=O)NC1=O)C2=O] | 2-[4-[4-[8-[3,5-difluoro-4- (morpholinomethyl)phenyl] quinoxalin-2-yl]pyrazol-1-yl]- 1-piperidyl]-N-[2-[2-[2-[2-[2- [2-[[2-(2,6-dioxo-3- piperidyl)-1,3-dioxo- isoindolin-4- yl]amino]ethoxy]ethoxy]ethoxy] ethoxy]ethoxy]ethyl]acetamide |
| JA-144 | 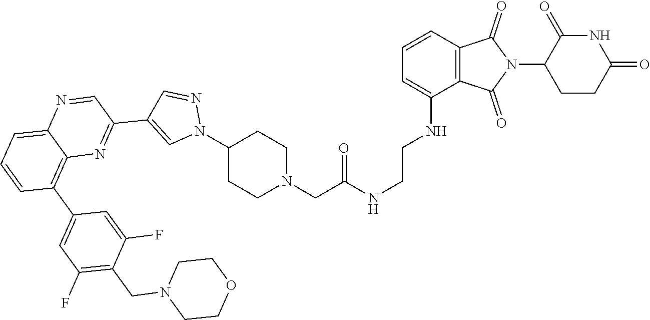 [SMILES: O=C(CN1CCC(n2cc(-c3cnc4cccc(-c5cc(F)c(CN6CCOCC6)c(F)c5)c4n3)cn2)CC1)NCCNc1cccc2c1C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(2-((2- (2,6-dioxopiperidin-3-yl)- 1,3-dioxoisoindolin-4- yl)amino)ethyl)acetamide |
| JA-145 | 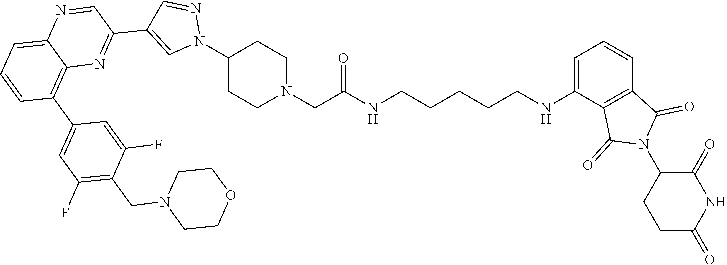 [SMILES: O=C(CN1CCC(n2cc(-c3cnc4cccc(-c5cc(F)c(CN6CCOCC6)c(F)c5)c4n3)cn2)CC1)NCCCCCNc1cccc2c1C(=O)N(C1CCC(=O)NC1=O)C2=O] | 2-[4-[4-[8-[3,5-difluoro-4- (morpholinomethyl)phenyl] quinoxalin-2-yl]pyrazol-1-yl]- 1-piperidyl]-N-[3-[[2-(2,6- dioxo-3-piperidyl)-1,3-dioxo- isoindolin-4- yl]amino]propyl]acetamide |
| JA-146 | 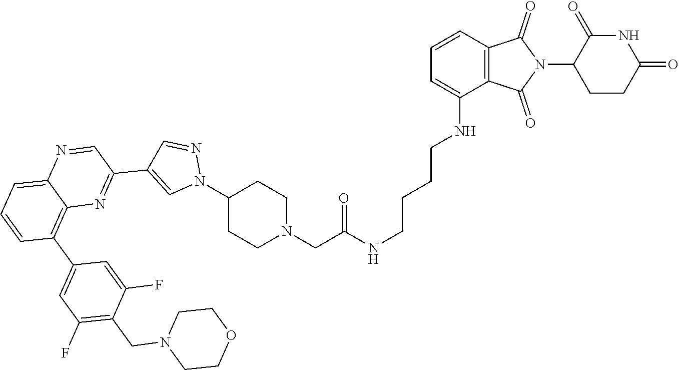 [SMILES: O=C(CN1CCC(n2cc(-c3cnc4cccc(-c5cc(F)c(CN6CCOCC6)c(F)c5)c4n3)cn2)CC1)NCCCCNc1cccc2c1C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(4-((2- (2,6-dioxopiperidin-3-yl)- 1,3-dioxoisoindolin-4- yl)amino)butyl)acetamide |
| JA-147 | 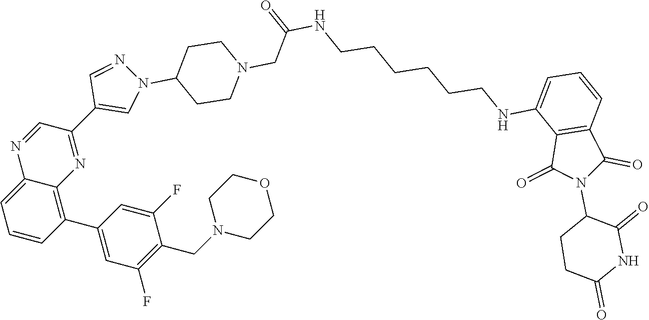 [SMILES: O=C(CN1CCC(n2cc(-c3cnc4cccc(-c5cc(F)c(CN6CCOCC6)c(F)c5)c4n3)cn2)CC1)NCCCCCCNc1cccc2c1C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(6-((2- (2,6-dioxopiperidin-3-yl)- 1,3-dioxoisoindolin-4- yl)amino)hexyl)acetamide |
| JA-148 | 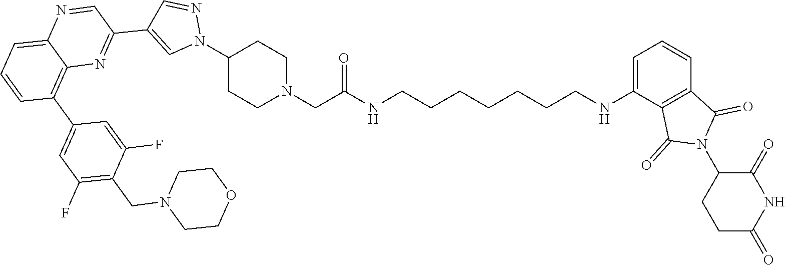 [SMILES: O=C(CN1CCC(n2cc(-c3cnc4cccc(-c5cc(F)c(CN6CCOCC6)c(F)c5)c4n3)cn2)CC1)NCCCCCCCNc1cccc2c1C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(7-((2- (2,6-dioxopiperidin-3-yl)- 1,3-dioxoisoindolin-4- yl)amino)heptyl)acetamide |
| JA-149 | 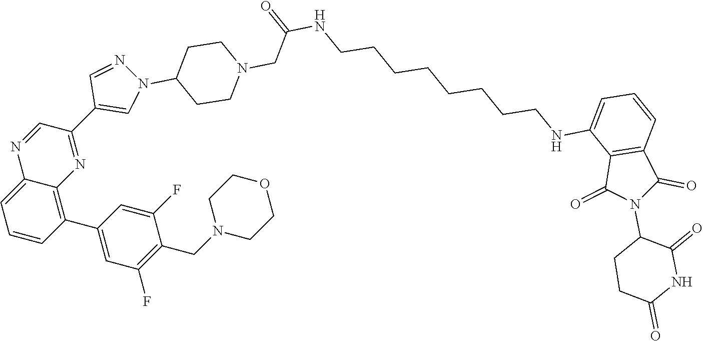 [SMILES: O=C(CN1CCC(n2cc(-c3cnc4cccc(-c5cc(F)c(CN6CCOCC6)c(F)c5)c4n3)cn2)CC1)NCCCCCCCCNc1cccc2c1C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(8-((2- (2,6-dioxopiperidin-3-yl)- 1,3-dioxoisoindolin-4- yl)amino)octyl)acetamide |
| JA-150 | 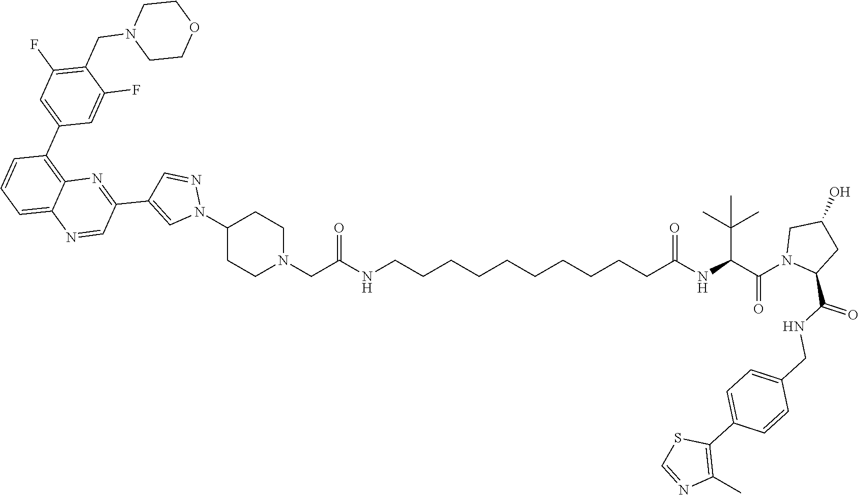 [SMILES: Cc1ncsc1-c1ccc(CNC(=O)[C@@H]2C[C@@H](O)CN2C(=O)[C@@H](NC(=O)CCCCCCCCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(11-(2-(4- (4-(8-(3,5-difluoro-4- (morpholinomethyl)phenyl) quinoxalin-2-yl)-1H-pyrazol- 1-yl)piperidin-1- yl)acetamido)undecanamido)- 3,3-dimethylbutanoyl)-4- hydroxy-N-(4-(4- methylthiazol-5- yl)benzyl)pyrrolidine-2- carboxamide |
| JA-151 | 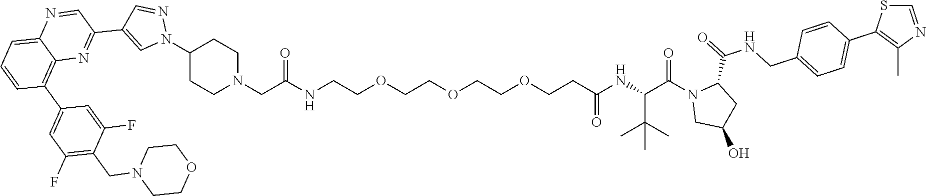 [SMILES: Cc1ncsc1-c1ccc(CNC(=O)[C@@H]2C[C@@H](O)CN2C(=O)[C@@H](NC(=O)CCOCCOCCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-17-(tert- butyl)-1-(4-(4-(8-(3,5- difluoro-4- (morpholinomethyl)phenyl) quinoxalin-2-yl)-1H-pyrazol- 1-yl)piperidin-1-yl)-2,15- dioxo-6,9,12-trioxa-3,16- diazaoctadecan-18-oyl)-4- hydroxy-N-(4-(4- methylthiazol-5- yl)benzyl)pyrrolidine-2- carboxamide |
| JA-152 | 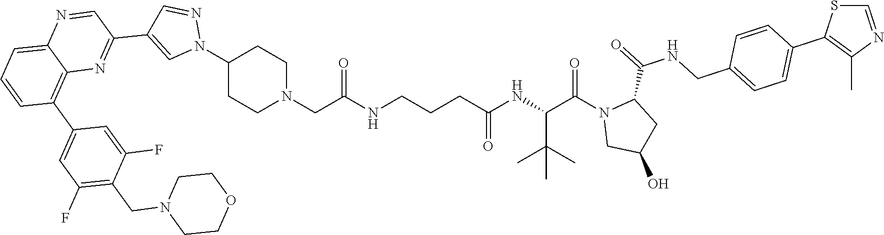 [SMILES: Cc1ncsc1-c1ccc(CNC(=O)[C@@H]2C[C@@H](O)CN2C(=O)[C@@H](NC(=O)CCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(4-(2-(4-(4- (8-(3,5-difluoro-4- (morpholinomethyl)phenyl) quinoxalin-2-yl)-1H-pyrazol- 1-yl)piperidin-1- yl)acetamido)butanamido)- 3,3-dimethylbutanoyl)-4- hydroxy-N-(4-(4- methylthiazol-5- yl)benzyl)pyrrolidine-2- carboxamide |
| JA-153 | 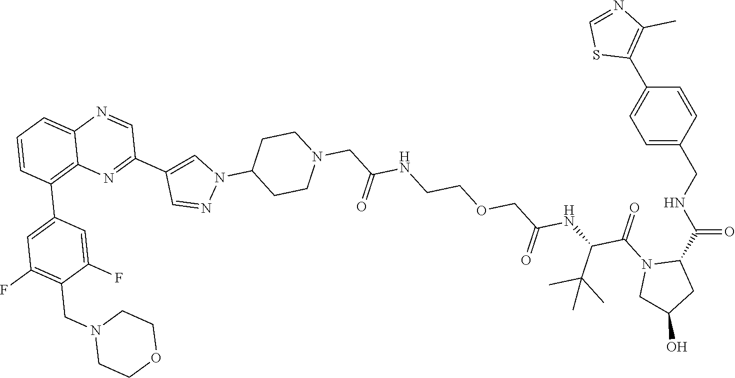 [SMILES: Cc1ncsc1-c1ccc(CNC(=O)[C@@H]2C[C@@H](O)CN2C(=O)[C@@H](NC(=O)COCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(2-(2-(2-(4- (4-(8-(3,5-difluoro-4- (morpholinomethyl)phenyl) quinoxalin-2-yl)-1H-pyrazol- 1-yl)piperidin-1- yl)acetamido)ethoxy)acetamido)- 3,3-dimethylbutanoyl)-4- hydroxy-N-(4-(4- methylthiazol-5- yl)benzyl)pyrrolidine-2- carboxamide |
| JA-154 | 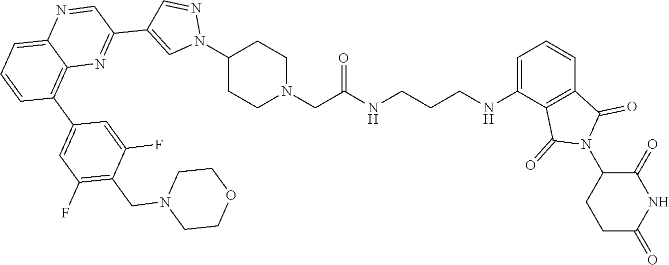 [SMILES: O=C(CN1CCC(n2cc(-c3cnc4cccc(-c5cc(F)c(CN6CCOCC6)c(F)c5)c4n3)cn2)CC1)NCCCNc1cccc2c1C(=O)N(C1CCC(=O)NC1=O)C2=O] | 2-(4-(4-8-(3,5-difluoro-4- (morpholinomethyl)phenyl) quinoxalin-2-yl)-1H-pyrazol- 1-yl)piperidin-1-yl)-N-(3-((2- (2,6-dioxopiperidin-3-yl)- 1,3-dioxoisoindolin-4- yl)amino)propyl)acetamide |
| JA-155 | 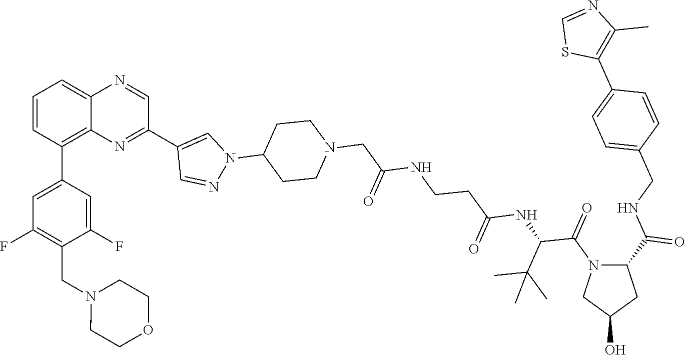 [SMILES: Cc1ncsc1-c1ccc(CNC(=O)[C@@H]2C[C@@H](O)CN2C(=O)[C@@H](NC(=O)CCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(3-(2-(4-(4- (8-(3,5-difluoro-4- (morpholinomethyl)phenyl) quinoxalin-2-yl)-1H-pyrazol- 1-yl)piperidin-1- yl)acetamido)propanamido)- 3,3-dimethylbutanoyl)-4- hydroxy-N-(4-(4- methylthiazol-5- yl)benzyl)pyrrolidine-2- carboxamide |
| JA-156 | 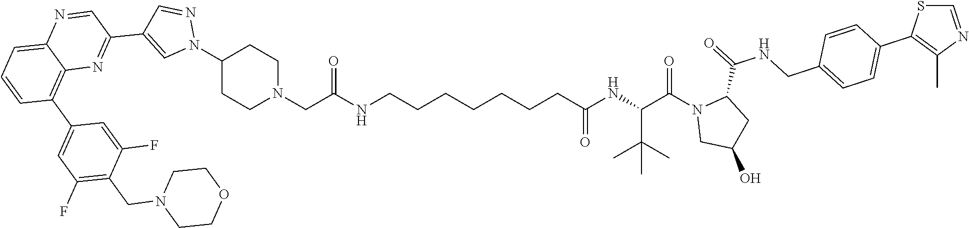 [SMILES: Cc1ncsc1-c1ccc(CNC(=O)[C@@H]2C[C@@H](O)CN2C(=O)[C@@H](NC(=O)CCCCCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(8-(2-(4-(4- (4-(8-(3,5-difluoro-4- (morpholinomethyl)phenyl) quinoxalin-2-yl)-1H-pyrazol- 1-yl)piperidin-1- yl)acetamido)octanamido)- 3,3-dimethylbutanoyl)-4- hydroxy-N-(4-(4- methylthiazol-5- yl)benzyl)pyrrolidine-2- carboxamide |
| JA-157 | 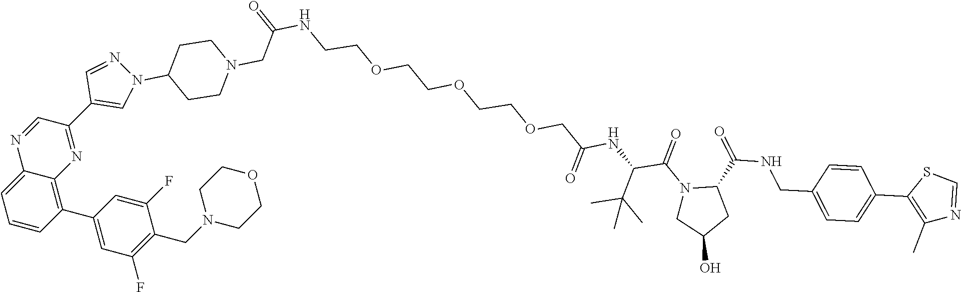 [SMILES: Cc1ncsc1-c1ccc(CNC(=O)[C@@H]2C[C@@H](O)CN2C(=O)[C@@H](NC(=O)COCCOCCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(tert-butyl)- 17-(4-(4-(8-(3,5-Difluoro-4- (morpholinomethyl)phenyl) quinoxalin-2-yl)-1H-pyrazol- 1-yl)piperidin-1-yl)-4,16- dioxo-6,9,12-trioxa-3,15- diazaheptadecanoyl)-4- hydroxy-N-(4-(4- methylthiazol-5- yl)benzyl)pyrrolidine-2- carboxamide |
| JA-158 | 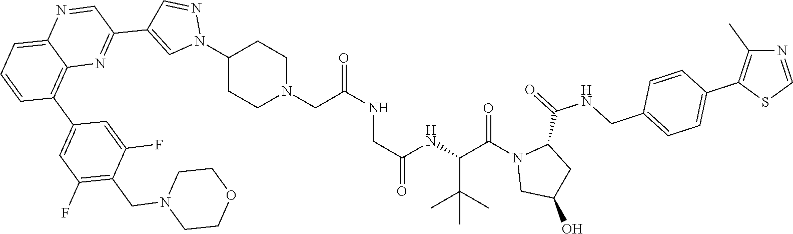 [SMILES: Cc1ncsc1-c1ccc(CNC(=O)[C@@H]2C[C@@H](O)CN2C(=O)[C@@H](NC(=O)CNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(2-(2-(4-(4- (8-(3,5-difluoro-4- (morpholinomethyl)phenyl) quinoxalin-2-yl)-1H-pyrazol- 1-yl)piperidin-1- yl)acetamido)acetamido)-3,3- dimethylbutanoyl)-4- hydroxy-N-(4-(4- methylthiazol-5- yl)benzyl)pyrrolidine-2- carboxamide |
| JA-159 | 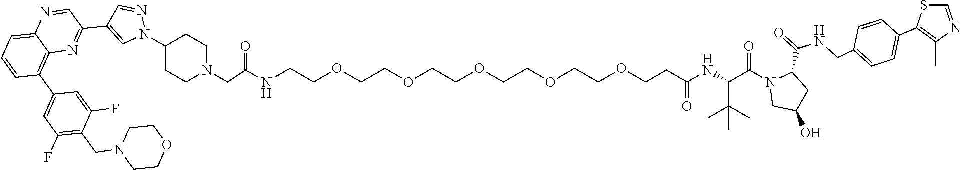 [SMILES: Cc1ncsc1-c1ccc(CNC(=O)[C@@H]2C[C@@H](O)CN2C(=O)[C@@H](NC(=O)CCOCCOCCOCCOCCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-23-(tert- butyl)-1-(4-(4-(8-(3,5- difluoro-4- (morpholinomethyl)phenyl) quinoxalin-2-yl)-1H-pyrazol- 1-yl)piperidin-1-yl)-2,21- dioxo-6,9,12,15,18-pentaoxa- 3,22-diazatetracosan-24-oyl)- 4-hydroxy-N-(4-(4- methylthiazol-5- yl)benzyl)pyrrolidine-2- |
| | | carboxamide |
| JA-160 | 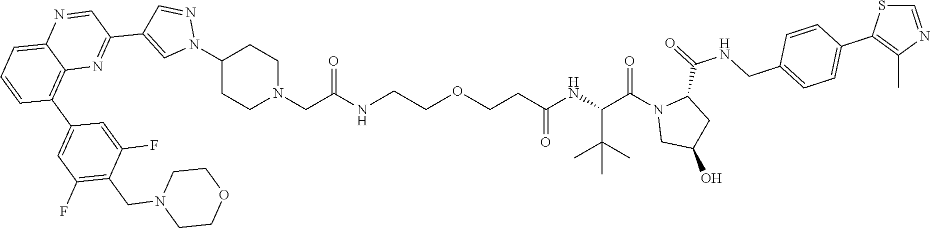 [SMILES: Cc1ncsc1-c1ccc(CNC(=O)[C@@H]2C[C@@H](O)CN2C(=O)[C@@H](NC(=O)CCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(3-(2-(2-(4- (4-(8-(3,5-difluoro-4- (morpholinomethyl)phenyl) quinoxalin-2-yl)-1H-pyrazol- 1-yl)piperidin-1- yl)acetamido)ethoxy) propanamido)-3,3- dimethylbutanoyl)-4- hydroxy-N-(4-(4- methylthiazol-5- yl)benzyl)pyrrolidine-2- carboxamide |
| JA-161 | 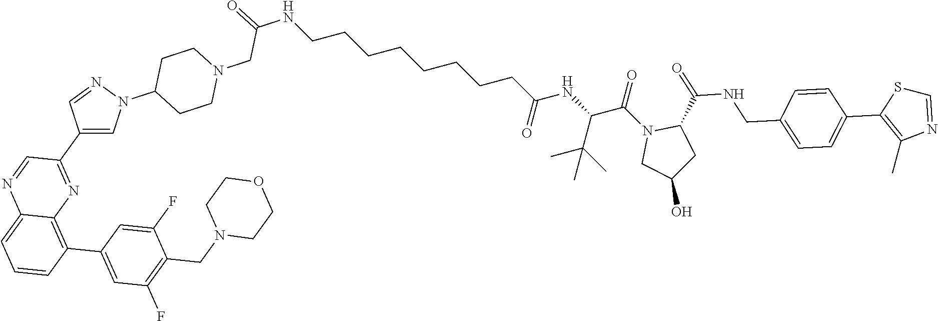 [SMILES: Cc1ncsc1-c1ccc(CNC(=O)[C@@H]2C[C@@H](O)CN2C(=O)[C@@H](NC(=O)CCCCCCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(9-(2-(4-(4- (8-(3,5-difluoro-4- (morpholinomethyl)phenyl) quinoxalin-2-yl)-1H-pyrazol- 1-yl)piperidin-1- yl)acetamido)nonanamido)- 3,3-dimethylbutanoyl)-4- hydroxy-N-(4-(4- methylthiazol-5- yl)benzyl)pyrrolidine-2- carboxamide |
| JA-162 | 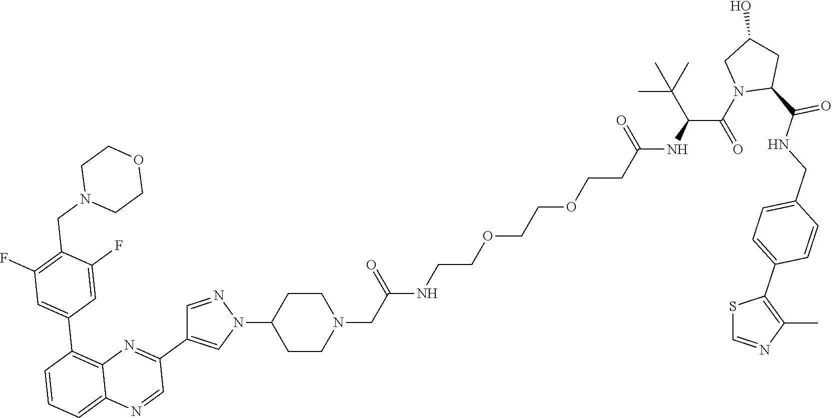 [SMILES: Cc1ncsc1-c1ccc(CNC(=O)[C@@H]2C[C@@H](O)CN2C(=O)[C@@H](NC(=O)CCOCCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-14-(tert- butyl)-1-(4-(4-(8-(3,5- difluoro-4- (morpholinomethyl)phenyl) quinoxalin-2-yl)-1H-pyrazol- 1-yl)piperidin-1-yl)-2,12- dioxo-6,9-dioxa-3,13- diazapentadecan-15-oyl)-4- hydroxy-N-(4-(4- methylthiazol-5- yl)benzyl)pyrrolidine-2- carboxamide |
| JA-163 | 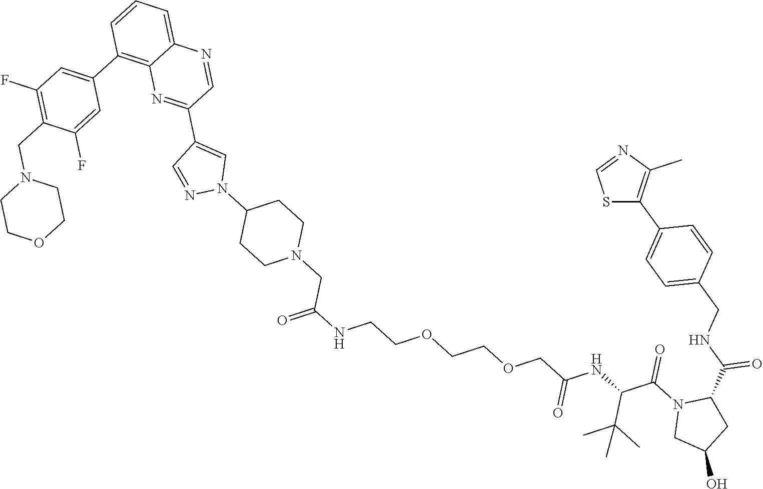 [SMILES: Cc1ncsc1-c1ccc(CNC(=O)[C@@H]2C[C@@H](O)CN2C(=O)[C@@H](NC(=O)COCCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(tert-butyl)- 14-(4-(4-(8-(3,5-difluoro-4- (morpholinomethyl)phenyl) quinoxalin-2-yl)-1H-pyrazol 1-yl)piperidin-1-yl)-4,13- dioxo-6,9-dioxa-3,12- diazatetradecanoyl)-4- hydroxy-N-(4-(4- methylthiazol-5- yl)benzyl)pyrrolidine-2- carboxamide |
| JA-164 | 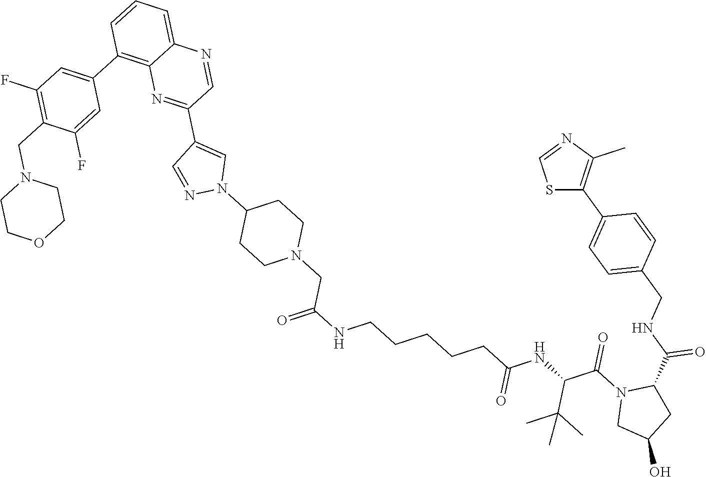 [SMILES: Cc1ncsc1-c1ccc(CNC(=O)[C@@H]2C[C@@H](O)CN2C(=O)[C@@H](NC(=O)CCCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(6-(2-(4-(4- (8-(3,5-difluoro-4- (morpholinomethyl)phenyl) quinoxalin-2-yl)-1H-pyrazol- 1-yl)piperidin-1- yl)acetamido)hexanamido)- 3,3-dimethylbutanoyl)-4- hydroxy-N-(4-(4- methylthiazol-5- yl)benzyl)pyrrolidine-2- carboxamide |
| JA-165 | 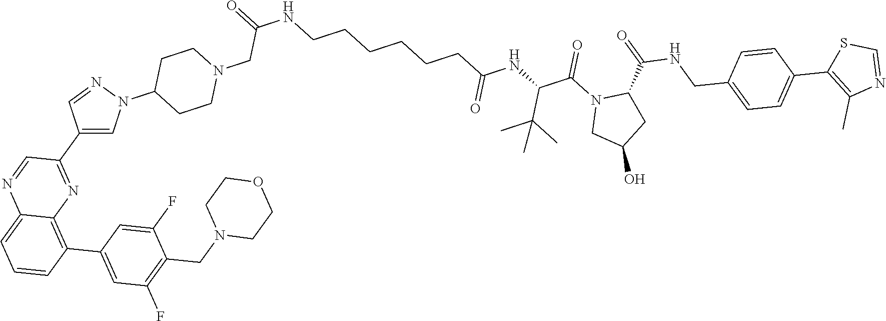 [SMILES: Cc1ncsc1-c1ccc(CNC(=O)[C@@H]2C[C@@H](O)CN2C(=O)[C@@H](NC(=O)CCCCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(7-(2-(4-(4- (8-(3,5-difluoro-4- (morpholinomethyl)phenyl) quinoxalin-2-yl)-1H-pyrazol- 1-yl)piperidin-1- yl)acetamido)heptanamido)- 3,3-dimethylbutanoyl)-4- hydroxy-N-(4-(4- methylthiazol-5- yl)benzyl)pyrrolidine-2- carboxamide |
| JA-166 | 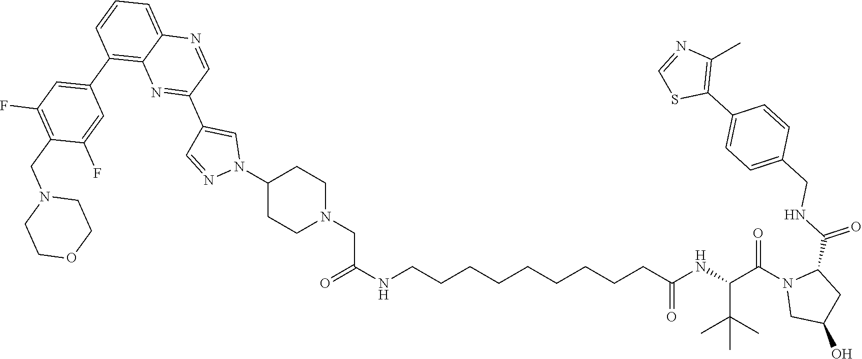 [SMILES: Cc1ncsc1-c1ccc(CNC(=O)[C@@H]2C[C@@H](O)CN2C(=O)[C@@H](NC(=O)CCCCCCCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(10-(2-(4- (4-(8-(3,5-difluoro-4- (morpholinomethyl)phenyl) quinoxalin-2-yl)-1H-pyrazol- 1-yl)piperidin-1- yl)acetamido)decanamido)- 3,3-dimethylbutanoyl)-4- hydroxy-N-(4-(4- methylthiazol-5- yl)benzyl)pyrrolidine-2- carboxamide |
| JA-167 | 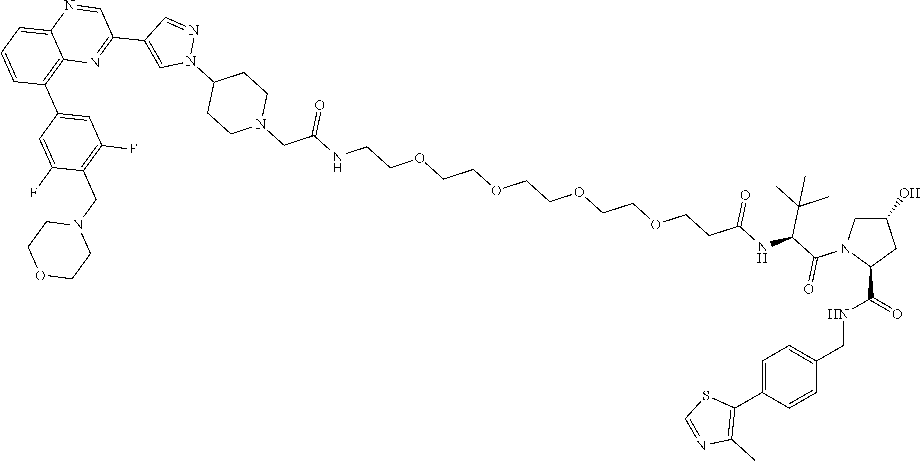 [SMILES: Cc1ncsc1-c1ccc(CNC(=O)[C@@H]2C[C@@H](O)CN2C(=O)[C@@H](NC(=O)CCOCCOCCOCCOCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-20-(tert- butyl)-1-(4-(4-(8-(3,5- difluoro-4- (morpholinomethyl)phenyl) quinoxalin-2-yl)-1H-pyrazol- 1-yl)piperidin-1-yl)-2,18- dioxo-6,9,12,15-tetraoxa- 3,19-diazahenicosan-21-oyl)- 4-hydroxy-N-(4-(4- methylthiazol-5- yl)benzyl)pyrrolidine-2- carboxamide |
| JA-168 | 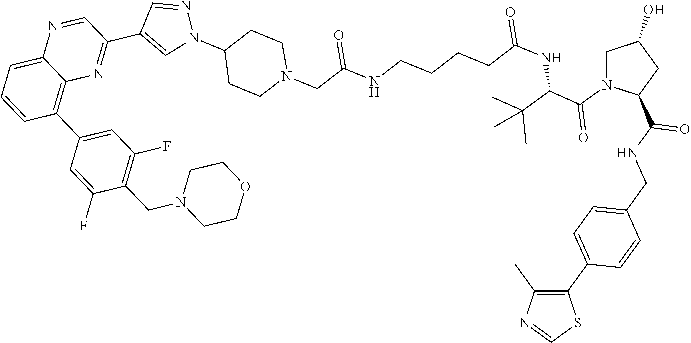 [SMILES: Cc1ncsc1-c1ccc(CNC(=O)[C@@H]2C[C@@H](O)CN2C(=O)[C@@H](NC(=O)CCCCNC(=O)CN2CCC(n3cc(-c4cnc5cccc(-c6cc(F)c(CN7CCOCC7)c(F)c6)c5n4)cn3)CC2)C(C)(C)C)cc1] | (2S,4R)-1-((S)-2-(5-(2-(4-(4- (8-(3,5-difluoro-4- (morpholinomethyl)phenyl) quinoxalin-2-yl)-1H-pyrazol- 1-yl)piperidin-1- yl)acetamido)pentanamido)- 3,3-dimethylbutanoyl)-4- hydroxy-N-(4-(4- methylthiazol-5- yl)benzyl)pyrrolidine-2- carboxamide |
| JA-169 | 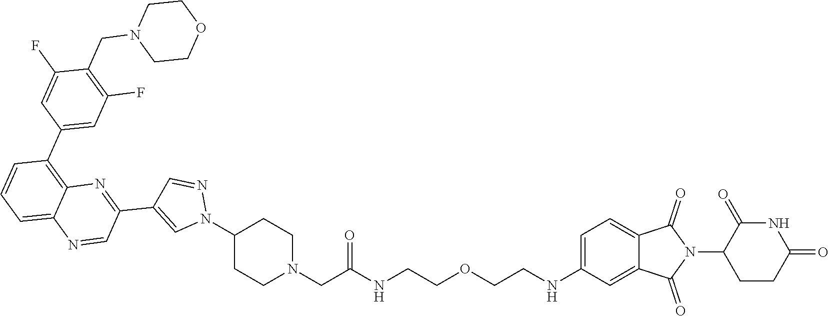 [SMILES: O=C(CN1CCC(n2cc(-c3cnc4cccc(-c5cc(F)c(CN6CCOCC6)c(F)c5)c4n3)cn2)CC1)NCCOCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(2-(2- ((2-(2,6-dioxopiperidin-3-yl)- 1,3-dioxoisoindolin-5- yl)amino)ethoxy)ethyl)acetamide |
| JA-170 | 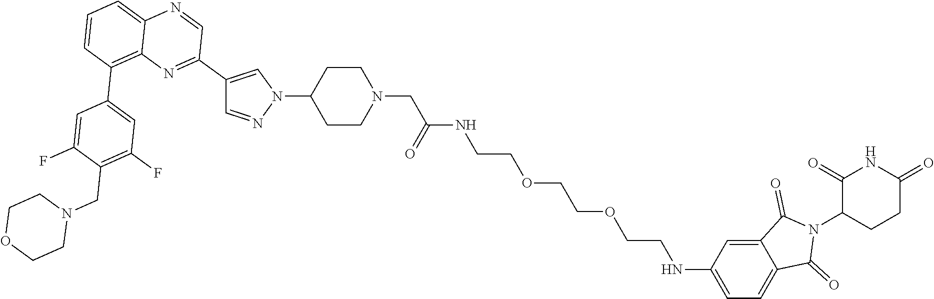 [SMILES: O=C(CN1CCC(n2cc(-c3cnc4cccc(-c5cc(F)c(CN6CCOCC6)c(F)c5)c4n3)cn2)CC1)NCCOCCOCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(2-(2- (2-((2-(2,6-dioxopiperidin-3- yl)-1,3-dioxoisoindolin-5- yl)amino)ethoxy)ethoxy)ethyl) acetamide |
| JA-171 | 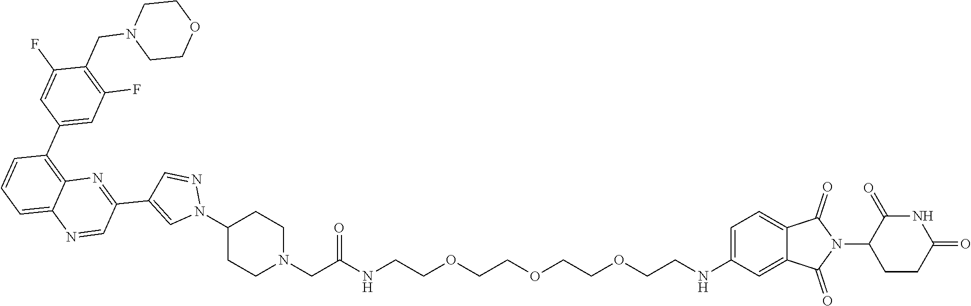 [SMILES: O=C(CN1CCC(n2cc(-c3cnc4cccc(-c5cc(F)c(CN6CCOCC6)c(F)c5)c4n3)cn2)CC1)NCCOCCOCCOCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(2-(2- (2-((2-(2,6- dioxopiperidin-3-yl)-1,3- dioxoisoindolin-5- yl)amino)ethoxy)ethoxy)ethoxy) ethyl)acetamide |
| JA-172 | 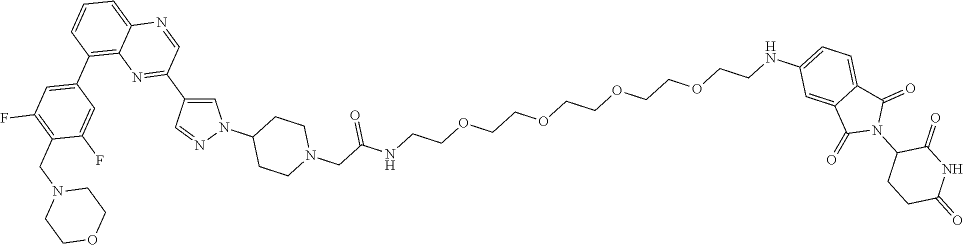 [SMILES: O=C(CN1CCC(n2cc(-c3cnc4cccc(-c5cc(F)c(CN6CCOCC6)c(F)c5)c4n3)cn2)CC1)NCCOCCOCCOCCOCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(14- ((2-(2,6-dioxopiperidin-3-yl)- 1,3-dioxoisoindolin-5- yl)amino)-3,6,9,12- tetraoxatetradecyl)acetamide |
| JA-173 | 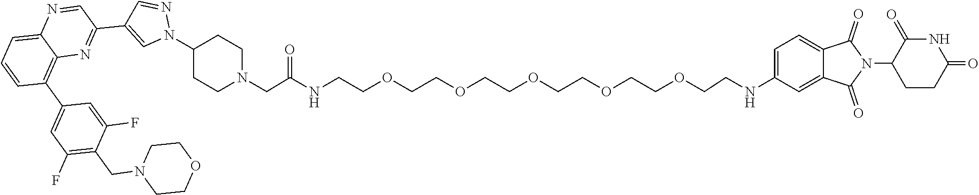 [SMILES: O=C(CN1CCC(n2cc(-c3cnc4cccc(-c5cc(F)c(CN6CCOCC6)c(F)c5)c4n3)cn2)CC1)NCCOCCOCCOCCOCCOCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(17- ((2-(2,6-dioxopiperidin-3-yl)- 1,3-dioxoisoindolin-5- yl)amino)-3,6,9,12,15- pentaoxaheptadecyl)acetamide |
| JA-174 | 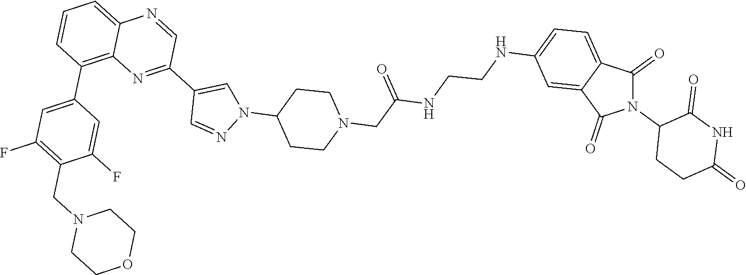 [SMILES: O=C(CN1CCC(n2cc(-c3cnc4cccc(-c5cc(F)c(CN6CCOCC6)c(F)c5)c4n3)cn2)CC1)NCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(2-((2- (2,6-dioxopiperidin-3-yl)- 1,3-dioxoisoindolin-5- yl)amino)ethyl)acetamide |
| JA-175 | 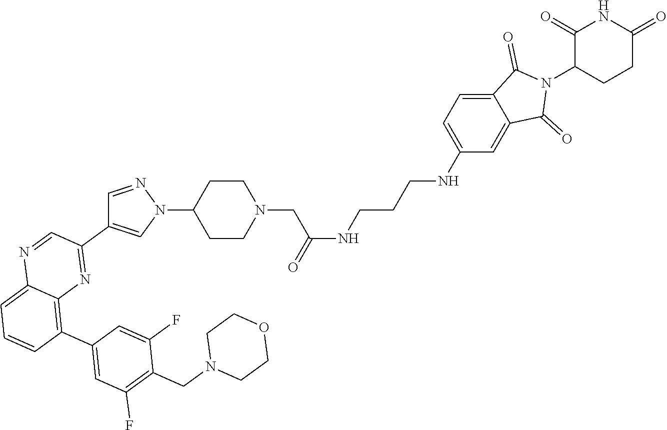 [SMILES: O=C(CN1CCC(n2cc(-c3cnc4cccc(-c5cc(F)c(CN6CCOCC6)c(F)c5)c4n3)cn2)CC1)NCCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(3-((2- (2,6-dioxopiperidin-3-yl)- 1,3-dioxoisoindolin-5- yl)amino)propyl)acetamide |
| JA-176 | 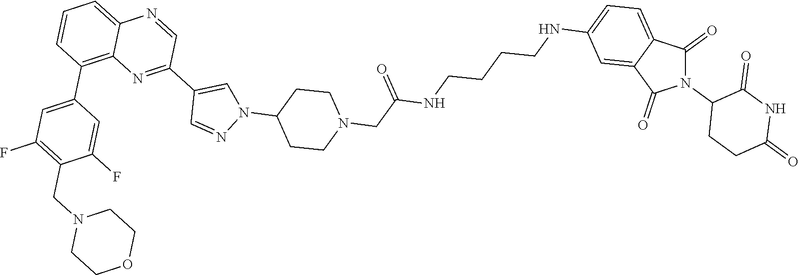 [SMILES: O=C(CN1CCC(n2cc(-c3cnc4cccc(-c5cc(F)c(CN6CCOCC6)c(F)c5)c4n3)cn2)CC1)NCCCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(4-((2- (2,6-dioxopiperidin-3-yl)- 1,3-dioxoisoindolin-5- yl)amino)butyl)acetamide |
| JA-177 | 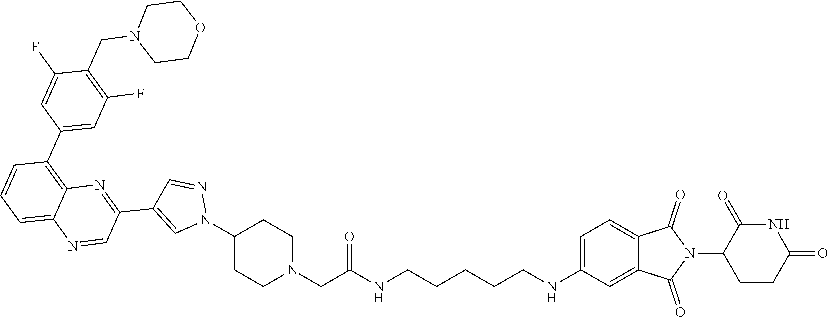 [SMILES: O=C(CN1CCC(n2cc(-c3cnc4cccc(-c5cc(F)c(CN6CCOCC6)c(F)c5)c4n3)cn2)CC1)NCCCCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(5-((2- (2,6-dioxopiperidin-3-yl)- 1,3-dioxoisoindolin-5- yl)amino)pentyl)acetamide |
| JA-178 | 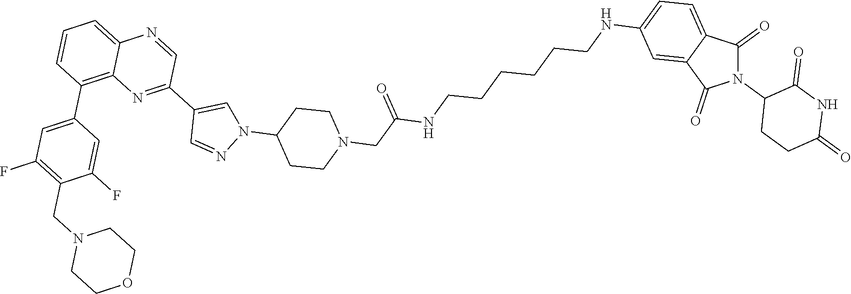 [SMILES: O=C(CN1CCC(n2cc(-c3cnc4cccc(-c5cc(F)c(CN6CCOCC6)c(F)c5)c4n3)cn2)CC1)NCCCCCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(6-((2- (2,6-dioxopiperidin-3-yl)- 1,3-dioxoisoindolin-5- yl)amino)hexyl)acetamide |
| JA-179 | 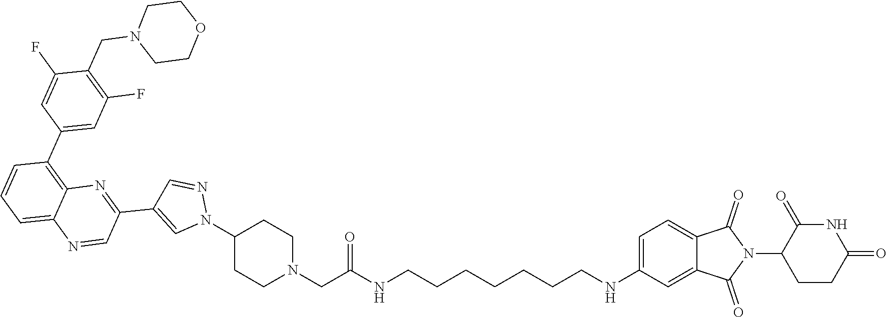 [SMILES: O=C(CN1CCC(n2cc(-c3cnc4cccc(-c5cc(F)c(CN6CCOCC6)c(F)c5)c4n3)cn2)CC1)NCCCCCCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(7-((2- (2,6-dioxopiperidin-3-yl)- 1,3-dioxoisoindolin-5- yl)amino)heptyl)acetamide |
| JA-180 | 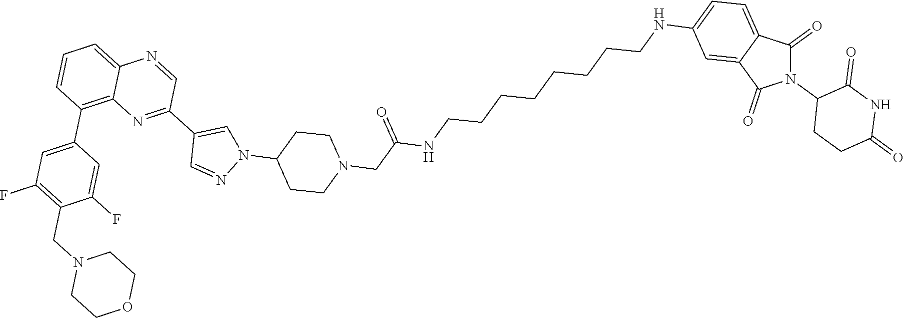 [SMILES: O=C(CN1CCC(n2cc(-c3cnc4cccc(-c5cc(F)c(CN6CCOCC6)c(F)c5)c4n3)cn2)CC1)NCCCCCCCCNc1ccc2c(c1)C(=O)N(C1CCC(=O)NC1=O)C2=O] | 2-(4-(4-(8-(3,5-difluoro-4- (morpholinomethyl)phenyl) quinoxalin-2-yl)-1H-pyrazol- 1-yl)piperidin-1-yl)-N-(8-((2- (2,6-dioxopiperidin-3-yl)- 1,3-dioxoisoindolin-5- yl)amino)octyl)acetamide |
| JA-181 | 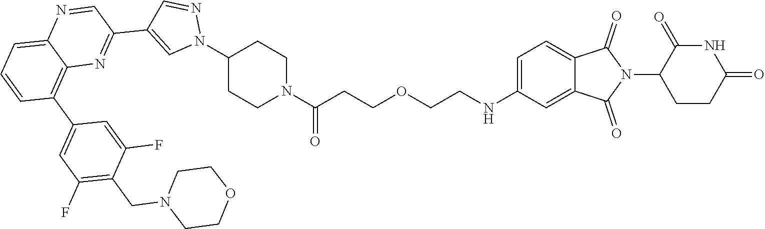 [SMILES: O=C1CCC(N2C(=O)c3ccc(NCCOCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((2-(3-(4-(4-(8-(3,5- difluoro-4- (morpholinomethyl)phenyl) quinoxalin-2-yl)-1H-pyrazol- 1-yl)piperidin-1-yl)-3- oxopropoxy)ethyl)amino)-2- (2,6-dioxopiperidin-3- yl)isoindoline-1,3-dione |
| JA-182 | 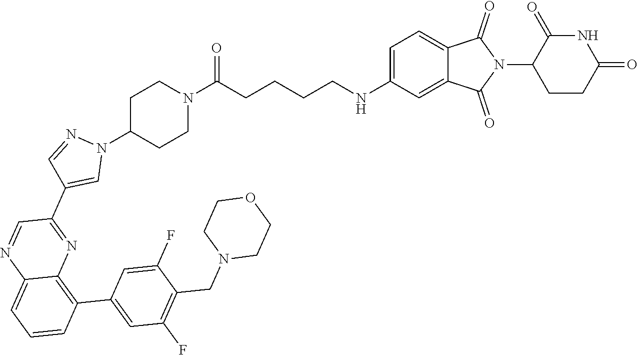 [SMILES: O=C1CCC(N2C(=O)c3ccc(NCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((5-(4-(4-(8-(3,5-difluoro- 4- (morpholinomethyl)phenyl) quinoxalin-2-yl)-1H-pyrazol- 1-yl)piperidin-1-yl)-5- oxopentyl)amino)-2-(2,6- dioxopiperidin-3- yl)isoindoline-1,3-dione |
| JA-183 | 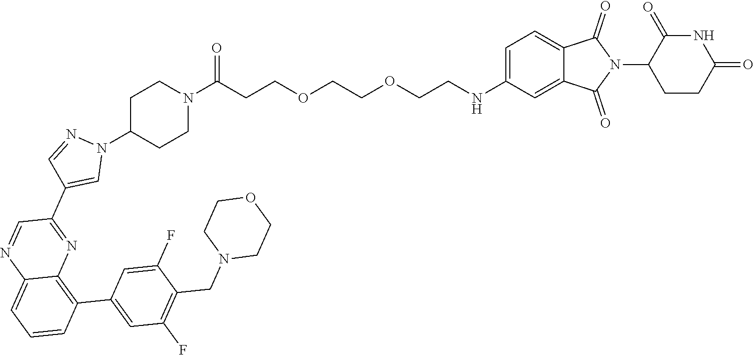 [SMILES: O=C1CCC(N2C(=O)c3ccc(NCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((2-(2-(3-(4-(4-(8-(3,5- difluoro-4- (morpholinomethyl)phenyl) quinoxalin-2-yl)-1H-pyrazol- 1-yl)piperidin-1-yl)-3- oxopropoxy)ethoxy)ethyl) amino)-2-(2,6-dioxopiperidin-3- yl)isoindoline-1,3-dione |
| JA-184 | 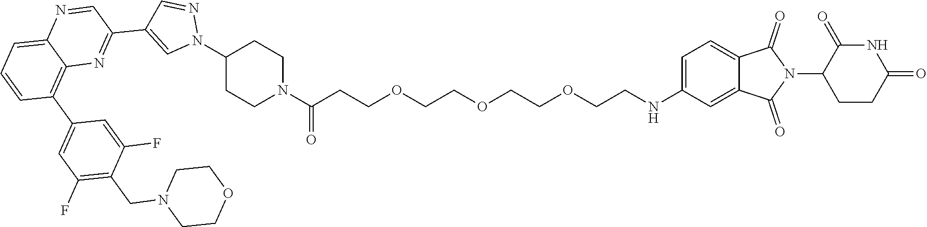 [SMILES: O=C1CCC(N2C(=O)c3ccc(NCCOCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((2-(2-(2-(3-(4-(4-(8-(3,5- difluoro-4- (morpholinomethyl)phenyl) quinoxalin-2-yl)-1H-pyrazol- 1-yl)piperidin-1-yl)-3- oxopropoxy)ethoxy)ethoxy) ethyl)amino)-2-(2,6- dioxopiperidin-3- yl)isoindoline-1,3-dione |
| JA-185 | 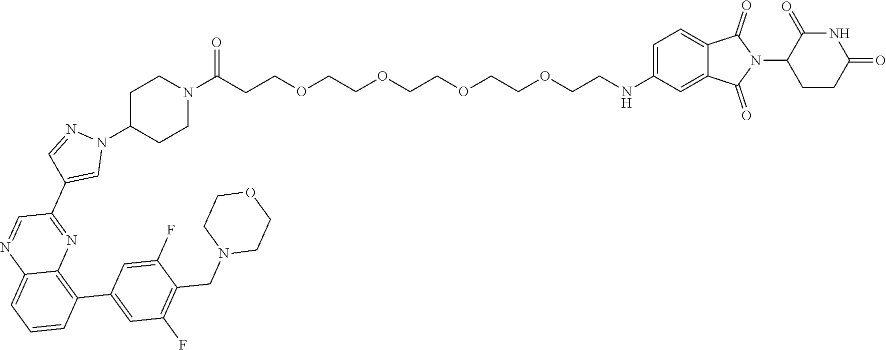 [SMILES: O=C1CCC(N2C(=O)c3ccc(NCCOCCOCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((15-(4-(4-(8-(3,5-difluoro- 4- (morpholinomethyl)phenyl) quinoxalin-2-yl)-1H-pyrazol- 1-yl)piperidin-1-yl)-15-oxo- 3,6,9,12- tetraoxapentadecyl)amino)-2- (2,6-dioxopiperidin-3- yl)isoindoline-1,3-dione |
| JA-186 | 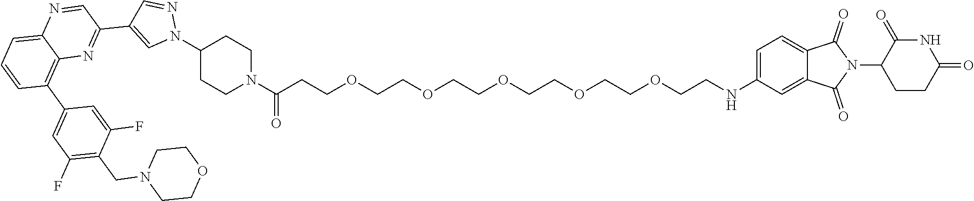 [SMILES: O=C1CCC(N2C(=O)c3ccc(NCCOCCOCCOCCOCCOCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((18-(4-(4-(8-(3,5-difluoro- 4- (morpholinomethyl)phenyl) quinoxalin-2-yl)-1H-pyrazol- 1-yl)piperidin-1-yl)-18-oxo- 3,6,9,12,15- pentaoxaoctadecyl)amino)-2- (2,6-dioxopiperidin-3- yl)isoindoline-1,3-dione |
| JA-187 | 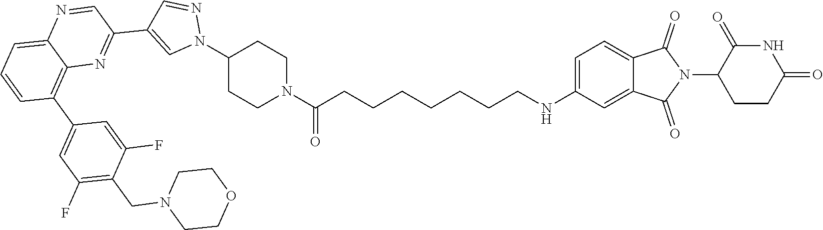 [SMILES: O=C1CCC(N2C(=O)c3ccc(NCCCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((8-(4-(4-(8-(3,5-difluoro- 4- (morpholinomethyl)phenyl) quinoxalin-2-yl)-1H-pyrazol- 1-yl)piperidin-1-yl)-8- oxooctyl)amino)-2-(2,6- dioxopiperidin-3- yl)isoindoline-1,3-dione |
| JA-188 | 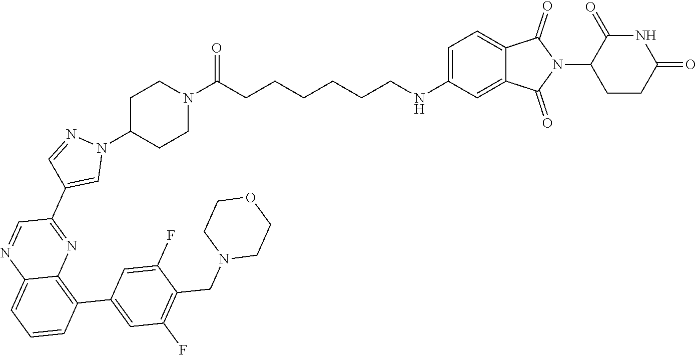 [SMILES: O=C1CCC(N2C(=O)c3ccc(NCCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((7-(4-(4-(8-(3,5-difluoro- 4- (morpholinomethyl)phenyl) quinoxalin-2-yl)-1H-pyrazol- 1-yl)piperidin-1-yl)-7- oxoheptyl)amino)-2-(2,6- dioxopiperidin-3- yl)isoindoline-1,3-dione |
| JA-189 | 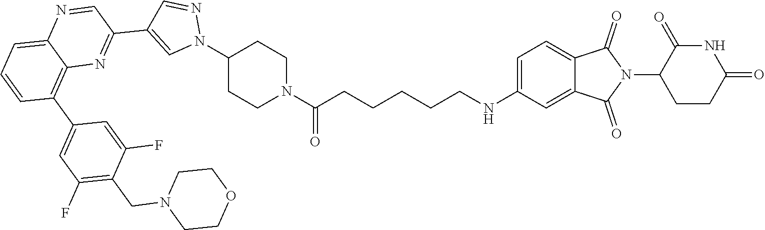 [SMILES: O=C1CCC(N2C(=O)c3ccc(NCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((6-(4-(4-(8-(3,5-difluoro- 4- (morpholinomethyl)phenyl) quinoxalin-2-yl)-1H-pyrazol- 1-yl)piperidin-1-yl)-6- oxohexyl)amino)-2-(2,6- dioxopiperidin-3- yl)isoindoline-1,3-dione |
| JA-190 | 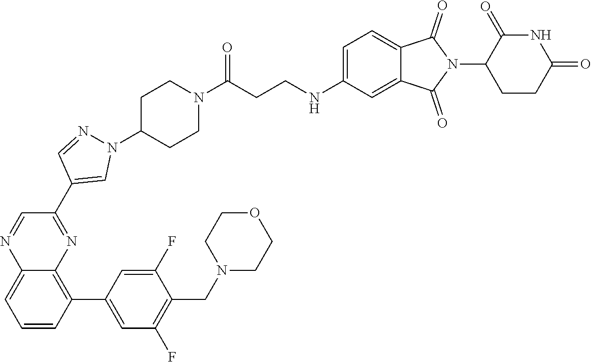 [SMILES: O=C1CCC(N2C(=O)c3ccc(NCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((3-(4-(4-(8-(3,5-difluoro- 4- (morpholinomethyl)phenyl) quinoxalin-2-yl)-1H-pyrazol- 1-yl)piperidin-1-yl)-3- oxopropyl)amino)-2-(2,6- dioxopiperidin-3- yl)isoindoline-1,3-dione |
| JA-191 | 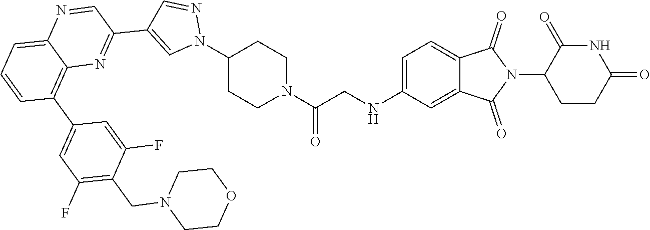 [SMILES: O=C1CCC(N2C(=O)c3ccc(NCC(=O)N4CCC(n5cc(-c6cnc7cccc(-c8cc(F)c(CN9CCOCC9)c(F)c8)c7n6)cn5)CC4)cc3C2=O)C(=O)N1] | 5-((2-(4-(4-(8-(3,5-difluoro- 4- (morpholinomethyl)phenyl) quinoxalin-2-yl)-1H-pyrazol- 1-yl)piperidin-1-yl)-2- oxoethyl)amino)-2-(2,6- dioxopiperidin-3- yl)isoindoline-1,3-dione |
| JA-192 | 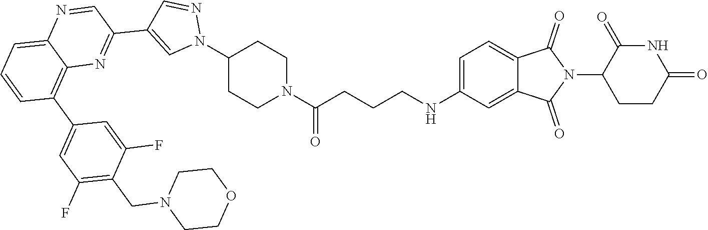 [SMILES: O=C1CCC(N2C(=O)c3ccc(NCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((4-(4-(4-(8-(3,5-difluoro- 4- (morpholinomethyl)phenyl) quinoxalin-2-yl)-1H-pyrazol- 1-yl)piperidin-1-yl)-4- oxobutyl)amino)-2-(2,6- dioxopiperidin-3- yl)isoindoline-1,3-dione |
| JA-193 | 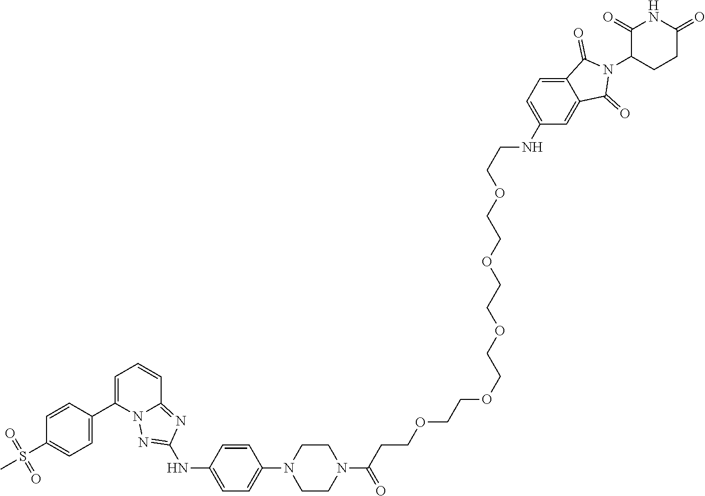 [SMILES: CS(=O)(=O)c1ccc(-c2cccc3nc(Nc4ccc(N5CCN(C(=O)CCOCCOCCOCCOCCOCCNc6ccc7c(c6)C(=O)N(C6CCC(=O)NC6=O)C7=O)CC5)cc4)nn23)cc1] | 2-(2,6-dioxopiperidin-3-yl)- 5-((18-(4-(4-((5-(4- (methylsulfonyl)phenyl)- [1,2,4]triazolo[1,5-a]pyridin- 2-yl)amino)phenyl)piperazin- 1-yl)-18-oxo-3,6,9,12,15- pentaoxaoctadecyl)amino) isoindoline-1,3-dione |
| JA-194 | 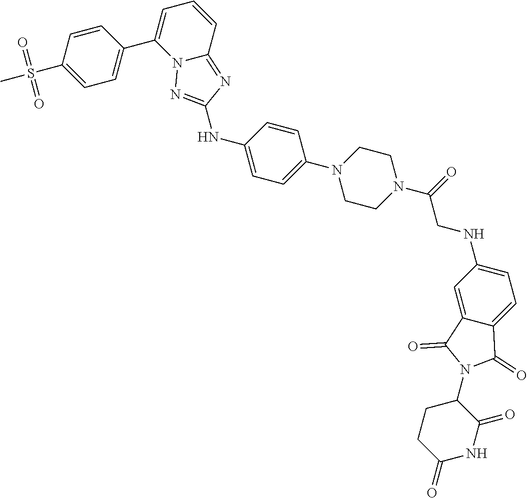 [SMILES: CS(=O)(=O)c1ccc(-c2cccc3nc(Nc4ccc(N5CCN(C(=O)CNc6ccc7c(c6)C(=O)N(C6CCC(=O)NC6=O)C7=O)CC5)cc4)nn23)cc1] | 2-(2,6-Dioxopiperidin-3-yl)- 5-((2-(4-(4-((5-(4- (methylsulfonyl)phenyl)- [1,2,4]triazolo[1,5-a]pyridin- 2-yl)amino)phenyl)piperazin- 1-yl)-2- oxoethyl)amino)isoindoline- 1,3-dione |
| JA-195 | 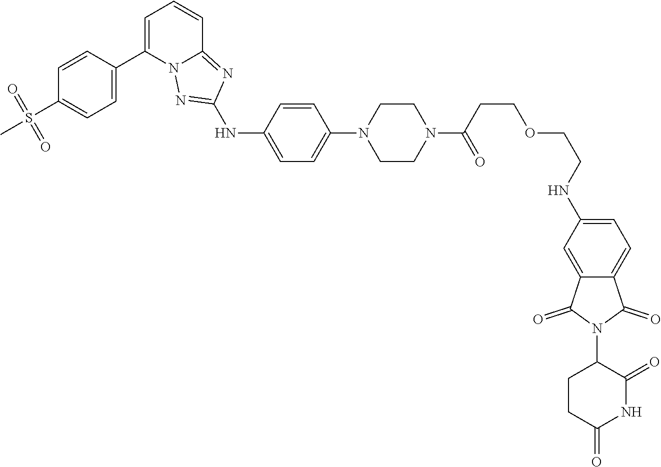 [SMILES: CS(=O)(=O)c1ccc(-c2cccc3nc(Nc4ccc(N5CCN(C(=O)CCOCCNc6ccc7c(c6)C(=O)N(C6CCC(=O)NC6=O)C7=O)CC5)cc4)nn23)cc1] | 2-(2,6-Dioxopiperidin-3-yl)- 5-((2-(3-(4-(4-((5-(4- (methylsulfonyl)phenyl)- [1,2,4]triazolo[1,5-a]pyridin- 2-yl)amino)phenyl)piperazin- 1-yl)-3- oxopropoxy)ethyl)amino) isoindoline-1,3-dione |
| JA-196 | 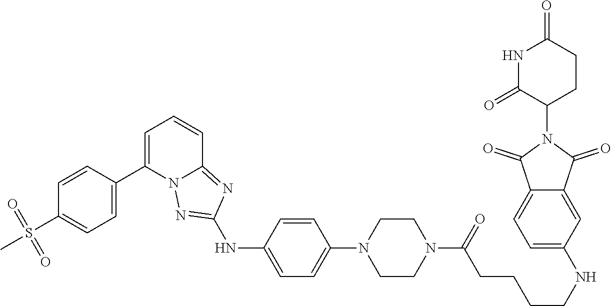 [SMILES: CS(=O)(=O)c1ccc(-c2cccc3nc(Nc4ccc(N5CCN(C(=O)CCCCNc6ccc7c(c6)C(=O)N(C6CCC(=O)NC6=O)C7=O)CC5)cc4)nn23)cc1] | 2-(2,6-Dioxopiperidin-3-yl)- 5-((5-(4-(4-((5-(4- (methylsulfonyl)phenyl)- [1,2,4]triazolo[1,5-a]pyridin- 2-yl)amino)phenyl)piperazin- 1-yl)-5- oxopentyl)amino)isoindoline- 1,3-dione |
| JA-197 | 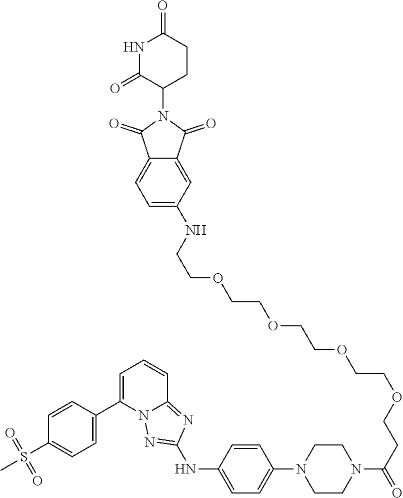 [SMILES: CS(=O)(=O)c1ccc(-c2cccc3nc(Nc4ccc(N5CCN(C(=O)CCOCCOCCOCCOCCNc6ccc7c(c6)C(=O)N(C6CCC(=O)NC6=O)C7=O)CC5)cc4)nn23)cc1] | 2-(2,6-Dioxopiperidin-3-yl)- 5-((15-(4-(4-((5-(4- (methylsulfonyl)phenyl)- [1,2,4]triazolo[1,5-a]pyridin- 2-yl)amino)phenyl)piperazin- 1-yl)-15-oxo-3,6,9,12- tetraoxapentadecyl)amino) isoindoline-1,3-dione |
| JA-198 | 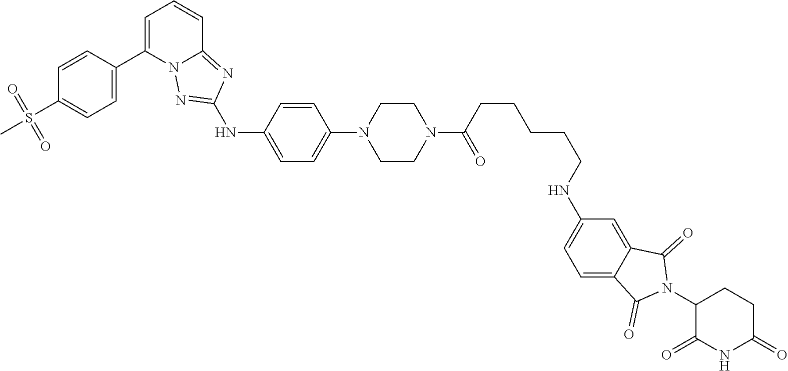 [SMILES: CS(=O)(=O)c1ccc(-c2cccc3nc(Nc4ccc(N5CCN(C(=O)CCCCCNc6ccc7c(c6)C(=O)N(C6CCC(=O)NC6=O)C7=O)CC5)cc4)nn23)cc1] | 2-(2,6-Dioxopiperidin-3-yl)- 5-((6-(4-(4-((5-(4- (methylsulfonyl)phenyl)- [1,2,4]triazolo[1,5-a]pyridin- 2-yl)amino)phenyl)piperazin- 1-yl)-6- oxoethyl)amino)isoindoline- 1,3-dione |
| JA-199 | 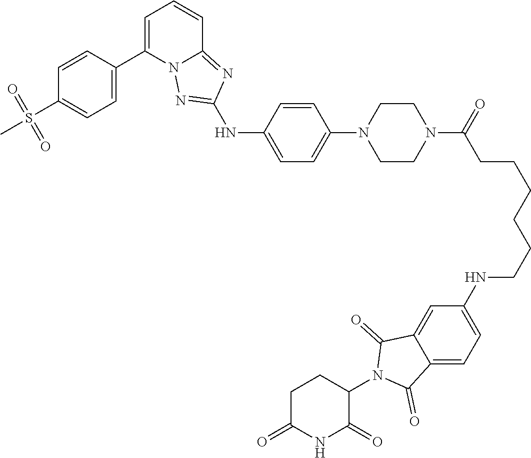 [SMILES: CS(=O)(=O)c1ccc(-c2cccc3nc(Nc4ccc(N5CCN(C(=O)CCCCCCNc6ccc7c(c6)C(=O)N(C6CCC(=O)NC6=O)C7=O)CC5)cc4)nn23)cc1] | 2-(2,6-Dioxopiperidin-3-yl)- 5-((7-(4-(4-((5-(4- (methylsulfonyl)phenyl)- [1,2,4]triazolo[1,5-a]pyridin- 2-yl)amino)phenyl)piperazin- 1-yl)-2- oxoheptyl)amino)isoindoline- 1,3-dione |
| JA-200 | 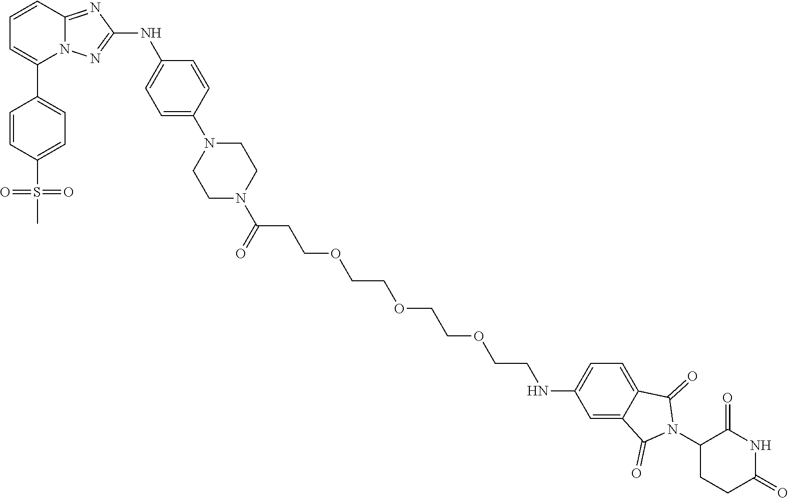 [SMILES: CS(=O)(=O)c1ccc(-c2cccc3nc(Nc4ccc(N5CCN(C(=O)CCOCCOCCOCCNc6ccc7c(c6)C(=O)N(C6CCC(=O)NC6=O)C7=O)CC5)cc4)nn23)cc1] | 2-(2,6-dioxopiperidin-3-yl)- 5-((2-(2-(2-(3-(4-(4-((5-(4- (methylsulfonyl)phenyl)- [1,2,4]triazolo[1,5-a]pyridin- 2-yl)amino)phenyl)piperazin- 1-yl)-3- oxopropoxy)ethoxy)ethoxy) ethyl)amino)isoindoline-1,3- dione |
| JA-201 | 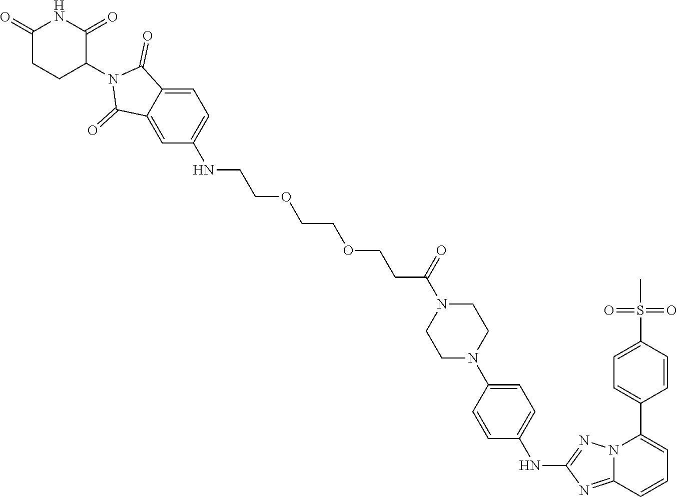 [SMILES: CS(=O)(=O)c1ccc(-c2cccc3nc(Nc4ccc(N5CCN(C(=O)CCOCCOCCNc6ccc7c(c6)C(=O)N(C6CCC(=O)NC6=O)C7=O)CC5)cc4)nn23)cc1] | 2-(2,6-dioxopiperidin-3-yl)- 5-((2-(2-(2-(3-(4-(4-((5-(4- (methylsulfonyl)phenyl)- [1,2,4]triazolo[1,5-a]pyridin- 2-yl)amino)phenyl)piperazin- 1-yl)-3- oxopropoxy)ethoxy)ethyl)amino) isoindoline-1,3-dione |
| JA-202 | 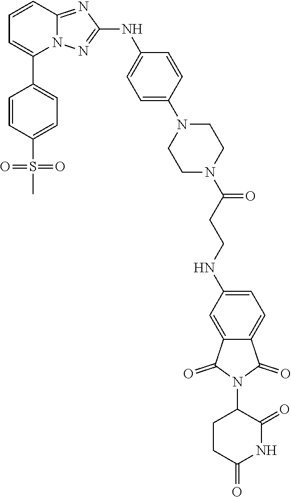 [SMILES: CS(=O)(=O)c1ccc(-c2cccc3nc(Nc4ccc(N5CCN(C(=O)CCNc6ccc7c(c6)C(=O)N(C6CCC(=O)NC6=O)C7=O)CC5)cc4)nn23)cc1] | 2-(2,6-dioxopiperidin-3-yl)- 5-((3-(4-(4-((5-(4- (methylsulfonyl)phenyl)- [1,2,4]triazolo[1,5-a]pyridin- 2-yl)amino)phenyl)piperazin- 1-yl)-3- oxopropyl)amino)isoindoline- 1,3-dione |
| JA-203 | 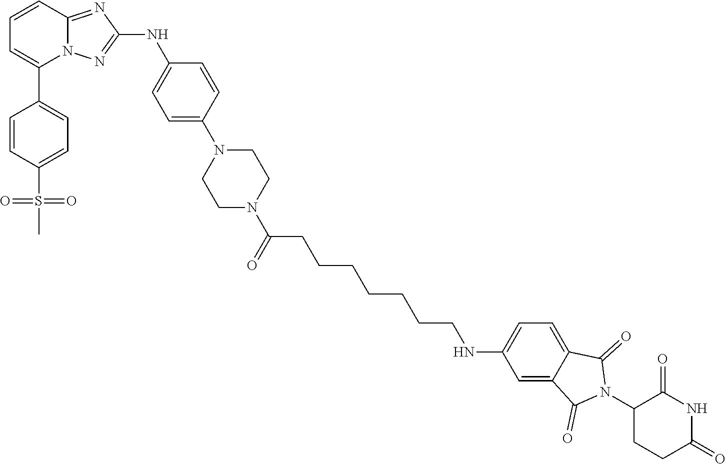 [SMILES: CS(=O)(=O)c1ccc(-c2cccc3nc(Nc4ccc(N5CCN(C(=O)CCCCCCCNc6ccc7c(c6)C(=O)N(C6CCC(=O)NC6=O)C7=O)CC5)cc4)nn23)cc1] | 2-(2,6-dioxopiperidin-3-yl)- 5-((8-(4-(4-((5-(4- (methylsulfonyl)phenyl)- [1,2,4]triazolo[1,5-a]pyridin- 2-yl)amino)phenyl)piperazin- 1-yl)-8- oxooctyl)amino)isoindoline- 1,3-dione |
| JA-204 | 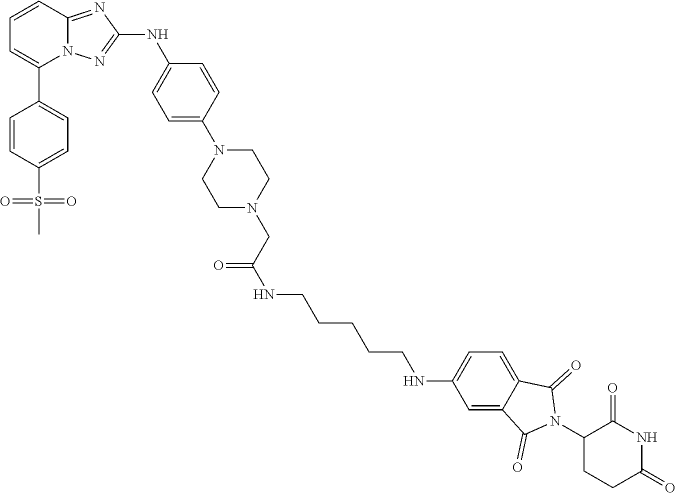 [SMILES: CS(=O)(=O)c1ccc(-c2cccc3nc(Nc4ccc(N5CCN(CC(=O)NCCCCCNc6ccc7c(c6)C(=O)N(C6CCC(=O)NC6=O)C7=O)CC5)cc4)nn23)cc1] | N-(5-((2-(2,6-dioxopiperidin- 3-yl)-1,3-dioxoisoindolin-5- yl)amino)pentyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-205 | 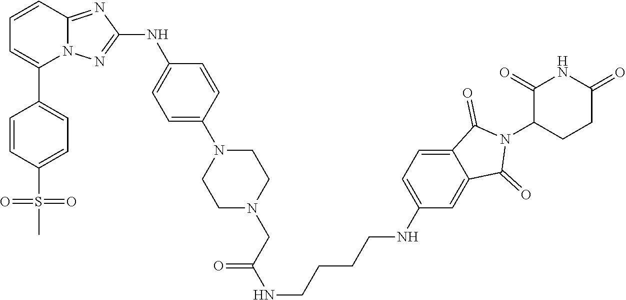 [SMILES: CS(=O)(=O)c1ccc(-c2cccc3nc(Nc4ccc(N5CCN(CC(=O)NCCCCNc6ccc7c(c6)C(=O)N(C6CCC(=O)NC6=O)C7=O)CC5)cc4)nn23)cc1] | N-(4-((2-(2,6-dioxopiperidin- 3-yl)-1,3-dioxoisoindolin-5- yl)amino)butyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-206 | 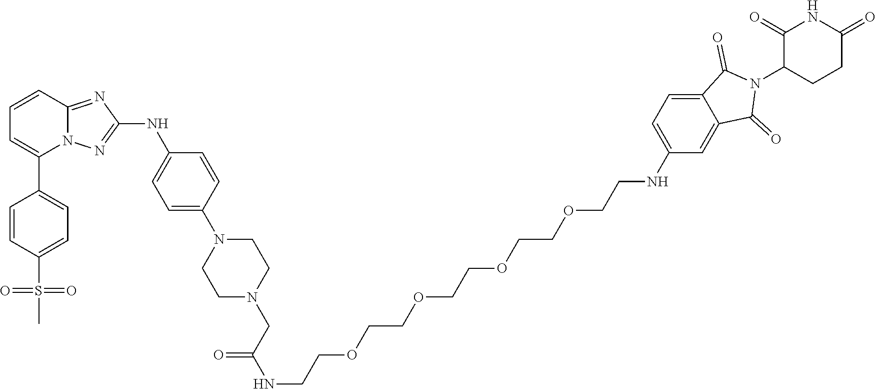 [SMILES: CS(=O)(=O)c1ccc(-c2cccc3nc(Nc4ccc(N5CCN(CC(=O)NCCOCCOCCOCCOCCNc6ccc7c(c6)C(=O)N(C6CCC(=O)NC6=O)C7=O)CC5)cc4)nn23)cc1] | N-(14-((2-(2,6- dioxopiperidin-3-yl)-1,3- dioxoisoindolin-5-yl)amino)- 3,6,9,12-tetraoxatetradecyl)- 2-(4-(4-((5-(4- (methylsulfonyl)phenyl)- [1,2,4]triazolo[1,5-a]pyridin- 2-yl)amino)phenyl)piperazin- 1-yl)acetamide |
| JA-207 | 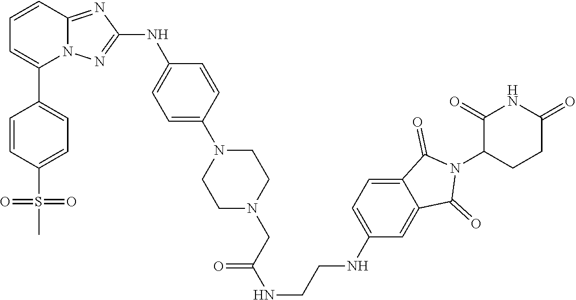 [SMILES: CS(=O)(=O)c1ccc(-c2cccc3nc(Nc4ccc(N5CCN(CC(=O)NCCNc6ccc7c(c6)C(=O)N(C6CCC(=O)NC6=O)C7=O)CC5)cc4)nn23)cc1] | N-(2-((2-(2,6-dioxopiperidin- 3-yl)-1,3-dioxoisoindolin-5- yl)amino)ethyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-208 | 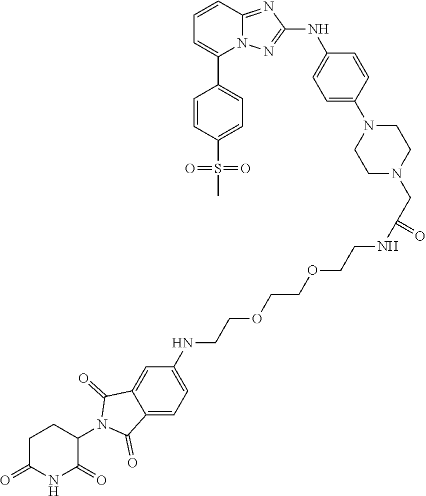 [SMILES: CS(=O)(=O)c1ccc(-c2cccc3nc(Nc4ccc(N5CCN(CC(=O)NCCOCCOCCNc6ccc7c(c6)C(=O)N(C6CCC(=O)NC6=O)C7=O)CC5)cc4)nn23)cc1] | N-(2-(2-(2-((2-(2,6- dioxopiperidin-3-yl)-1,3- dioxoisoindolin-5- yl)amino)ethoxy)ethoxy)ethyl)- 2-(4-(4-((5-(4- (methylsulfonyl)phenyl)- [1,2,4]triazolo[1,5-a]pyridin- 2-yl)amino)phenyl)piperazin- 1-yl)acetamide |
| JA-209 | 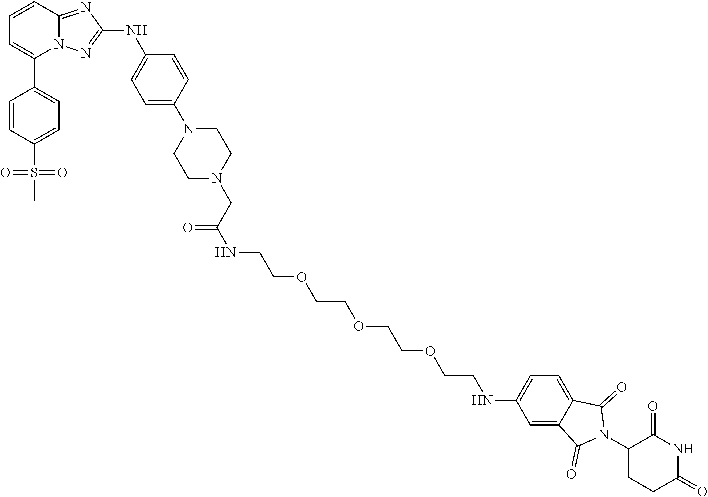 [SMILES: CS(=O)(=O)c1ccc(-c2cccc3nc(Nc4ccc(N5CCN(CC(=O)NCCOCCOCCOCCNc6ccc7c(c6)C(=O)N(C6CCC(=O)NC6=O)C7=O)CC5)cc4)nn23)cc1] | N-(2-(2-(2-(2-((2-(2,6- dioxopiperidin-3-yl)-1,3- dioxoisoidolin-5- yl)amino)ethoxy)ethoxy)ethoxy) ethyl)-2-(4-(4-((5-(4- (methylsulfonyl)phenyl)- [1,2,4]triazolo[1,5-a]pyridin- 2-yl)amino)phenyl)piperazin- 1-yl)acetamide |
| JA-210 | 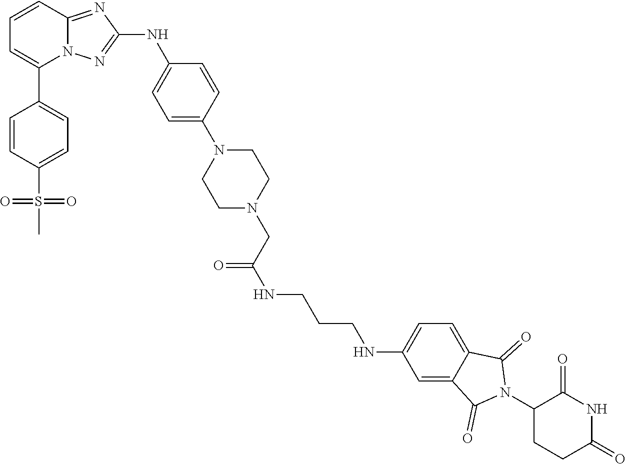 [SMILES: CS(=O)(=O)c1ccc(-c2cccc3nc(Nc4ccc(N5CCN(CC(=O)NCCCNc6ccc7c(c6)C(=O)N(C6CCC(=O)NC6=O)C7=O)CC5)cc4)nn23)cc1] | N-(3-((2-(2,6-dioxopiperidin- 3-yl)-1,3-dioxoisoindolin-5- yl)amino)propyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-211 | 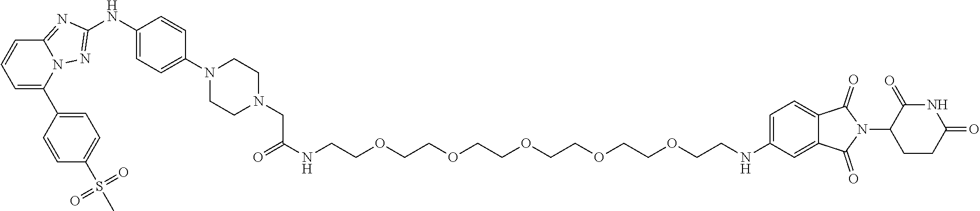 [SMILES: CS(=O)(=O)c1ccc(-c2cccc3nc(Nc4ccc(N5CCN(CC(=O)NCCOCCOCCOCCOCCOCCNc6ccc7c(c6)C(=O)N(C6CCC(=O)NC6=O)C7=O)CC5)cc4)nn23)cc1] | N-(17-((2-(2,6- dioxopiperidin-3-yl)-1,3- dioxoisoindolin-5-yl)amino)- 3,6,9,12,15- pentaoxaheptadecyl)-2-(4-(4- ((5-(4- (methylsulfonyl)phenyl)- [1,2,4]triazolo[1,5-a]pyridin- 2-yl)amino)phenyl)piperazin- 1-yl)acetamide |
| JA-212 | 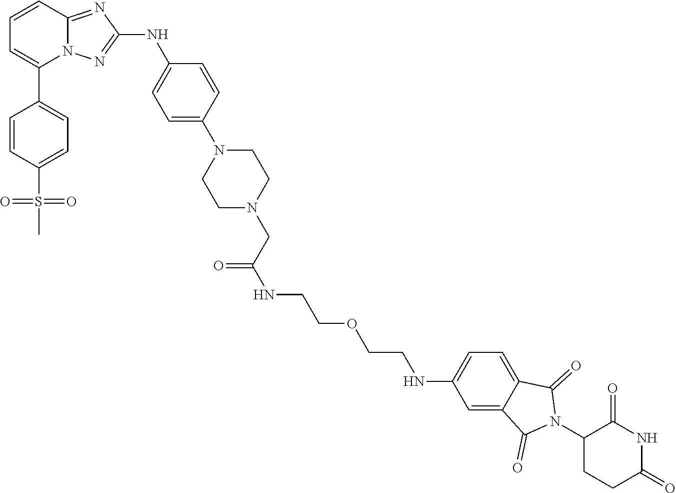 [SMILES: CS(=O)(=O)c1ccc(-c2cccc3nc(Nc4ccc(N5CCN(CC(=O)NCCOCCNc6ccc7c(c6)C(=O)N(C6CCC(=O)NC6=O)C7=O)CC5)cc4)nn23)cc1] | N-(2-(2-((2-(2,6- dioxopiperidin-3-yl)-1,3- dioxoisoindolin-5- yl)amino)ethoxy)ethyl)-2-(4- (4-((5-(4- (methylsulfonyl)phenyl)- [1,2,4]triazolo[1,5-a]pyridin- 2-yl)amino)phenyl)piperazin- 1-yl)acetamide |
| JA-213 | 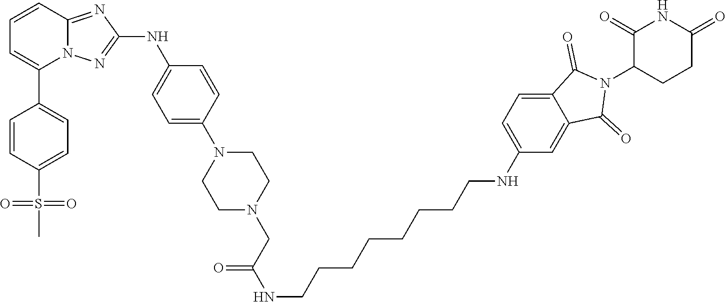 [SMILES: CS(=O)(=O)c1ccc(-c2cccc3nc(Nc4ccc(N5CCN(CC(=O)NCCCCCCCCNc6ccc7c(c6)C(=O)N(C6CCC(=O)NC6=O)C7=O)CC5)cc4)nn23)cc1] | N-(8-((2-(2,6-dioxopiperidin- 3-yl)-1,3-dioxoisoindolin-5- yl)amino)octyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-214 | 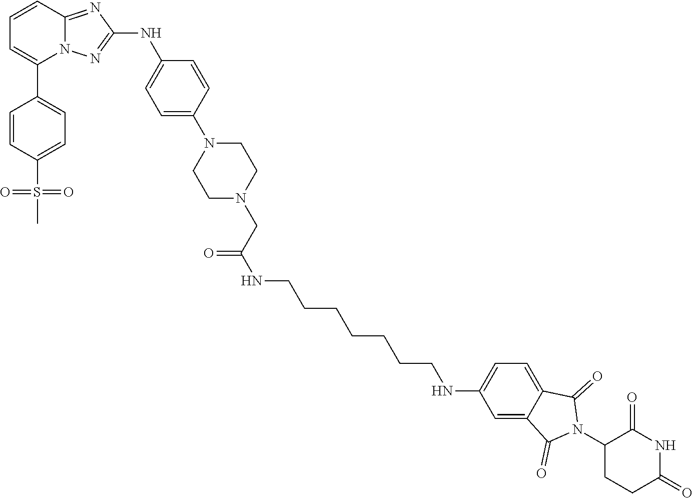 [SMILES: CS(=O)(=O)c1ccc(-c2cccc3nc(Nc4ccc(N5CCN(CC(=O)NCCCCCCCNc6ccc7c(c6)C(=O)N(C6CCC(=O)NC6=O)C7=O)CC5)cc4)nn23)cc1] | N-(7-((2-(2,6-dioxopiperidin- 3-yl)-1,3-dioxoisoindolin-5- yl)amino)heptyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-215 | 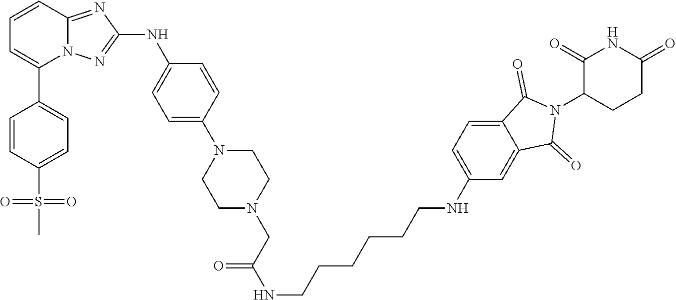 [SMILES: CS(=O)(=O)c1ccc(-c2cccc3nc(Nc4ccc(N5CCN(CC(=O)NCCCCCCNc6ccc7c(c6)C(=O)N(C6CCC(=O)NC6=O)C7=O)CC5)cc4)nn23)cc1] | N-(6-((2-(2,6-dioxopiperidin- 3-yl)-1,3-dioxoisoindolin-5- yl)amino)hexyl)-2-(4-(4-((5- (4-(methylsulfonyl)phenyl)- [1,2,4]triazolo[1,5-a]pyridin- 2-yl)amino)phenyl)piperazin- 1-yl)acetamide |
| JA-216 | 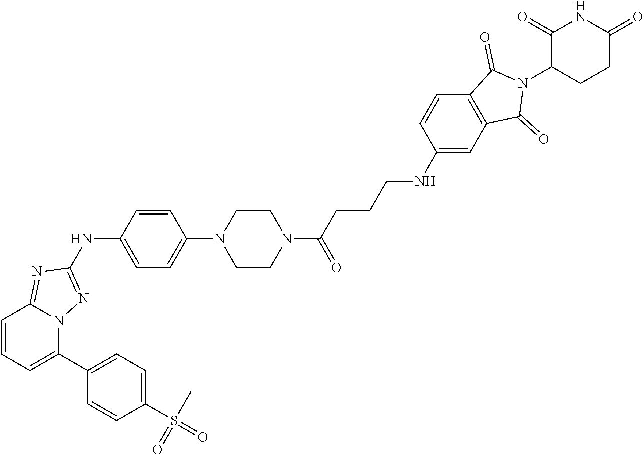 [SMILES: CS(=O)(=O)c1ccc(-c2cccc3nc(Nc4ccc(N5CCN(C(=O)CCCNc6ccc7c(c6)C(=O)N(C6CCC(=O)NC6=O)C7=O)CC5)cc4)nn23)cc1] | 2-(2,6-dioxopiperidin-3-yl)- 5-((4-(4-(4-((5-(4- (methylsulfonyl)phenyl)- [1,2,4]triazolo[1,5-a]pyridin- 2-yl)amino)phenyl)piperazin- 1-yl)-4- oxobutyl)amino)isoindoline- 1,3-dione |
| JA-217 | 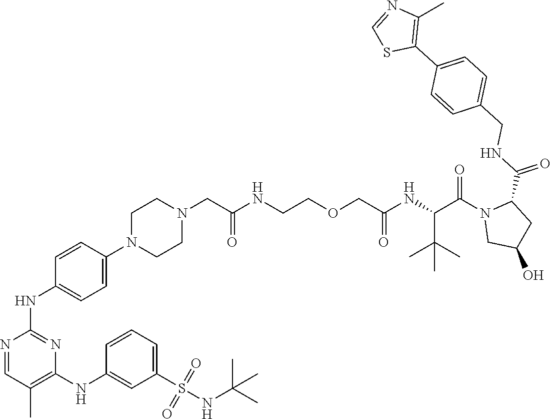 [SMILES: Cc1cnc(Nc2ccc(N3CCN(CC(=O)NCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(2-(2-(2-(4- (4-((4-((3-(N-(tert- butyl)sulfamoyl)phenyl)amino)- 5-methylpyrimidin-2- yl)amino)phenyl)piperazin-1- yl)acetamido)ethoxy)acetamido)- 3,3-dimethylbutanoyl)-4- hydroxy-N-(4-(4- methylthiazol-5- yl)benzyl)pyrrolidine-2- carboxamide |
| JA-218 | 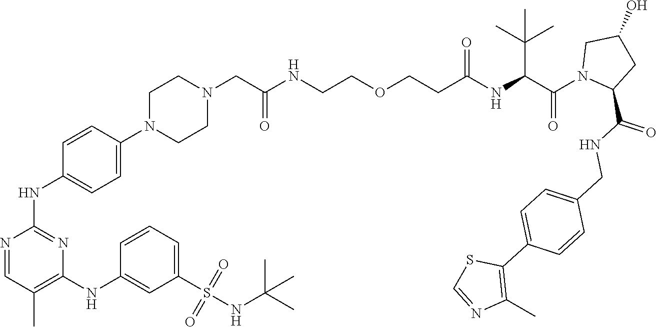 [SMILES: Cc1cnc(Nc2ccc(N3CCN(CC(=O)NCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(3-(2-(2-(4- (4-((4-((3-(N-(tert- butyl)sulfamoyl)phenyl)amino)- 5-methylpyrimidin-2- yl)amino)phenyl)piperazin-1- yl)acetamido)ethoxy) propanamido)-3,3- dimethylbutanoyl)-4- hydroxy-N-(4-(4- methylthiazol-5- yl)benzyl)pyrrolidine-2- carboxamide |
| JA-219 | 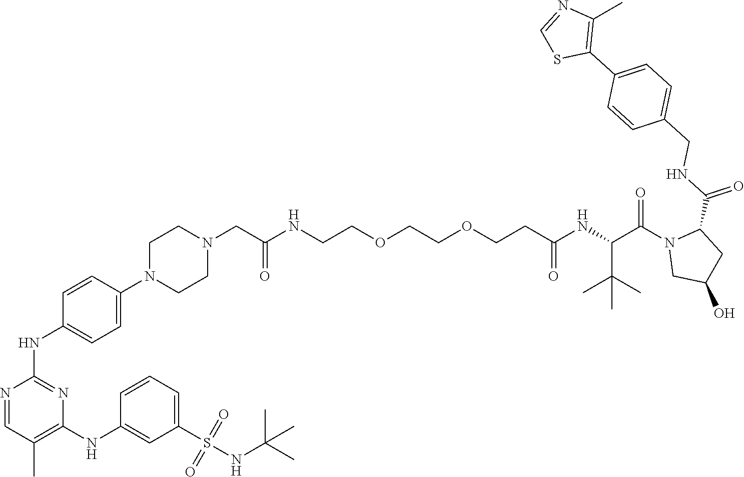 [SMILES: Cc1cnc(Nc2ccc(N3CCN(CC(=O)NCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-14-(tert- butyl)-1-(4-(4-((4-((3-(N- (tert- butyl)sulfamoyl)phenyl)amino)- 5-methylpyrimidin-2- yl)amino)phenyl)piperazin-1- yl)-2,12-dioxo-6,9-dioxa- 3,13-diazapentadecan-15- oyl)-4-hydroxy-N-(4-(4- methylthiazol-5- yl)benzyl)pyrrolidine-2- carboxamide |
| JA-220 | 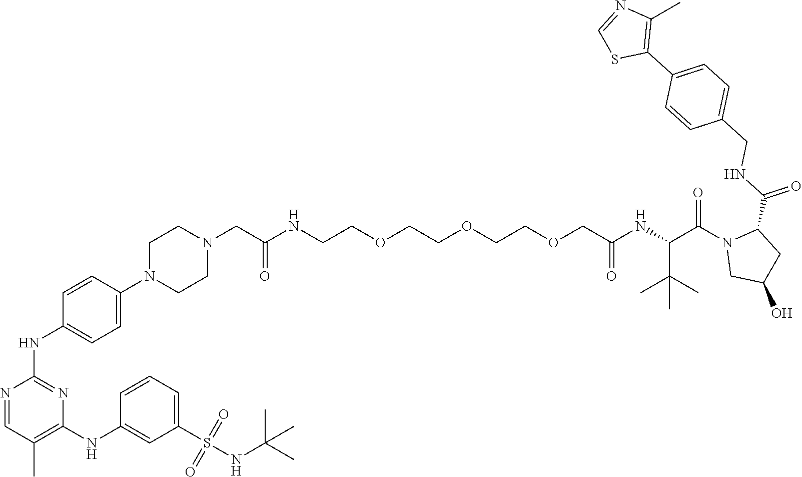 [SMILES: Cc1cnc(Nc2ccc(N3CCN(CC(=O)NCCOCCOCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 17-(4-(4-((4-((3-(N-(tert- butyl)sulfamoyl)phenyl)amino)- 5-methylpyrimidin-2- yl)amino)phenyl)piperazin-1- yl)-4,16-dioxo-6,9,12-trioxa- 3,15-diazaheptadecanoyl)-4- hydroxy-N-(4-(4- methylthiazol-5- yl)benzyl)pyrrolidine-2- carboxamide |
| JA-221 | 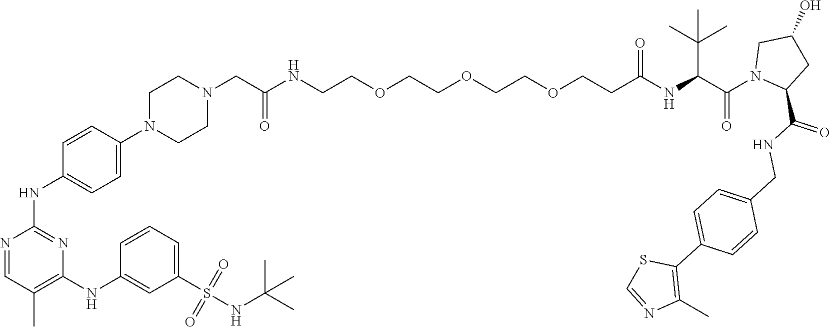 [SMILES: Cc1cnc(Nc2ccc(N3CCN(CC(=O)NCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-17-(tert- butyl)-1-(4-(4-((4-((3-(N- (tert- butyl)sulfamoyl)phenyl)amino)- 5-methylpyrimidin-2- yl)amino)phenyl)piperazin-1- yl)-2,15-dioxo-6,9,12-trioxa- 3,16-diazaoctadecan-18-oyl)- 4-hydroxy-N-(4-(4- methylthiazol-5- yl)benzyl)pyrrolidine-2- carboxamide |
| JA-222 | 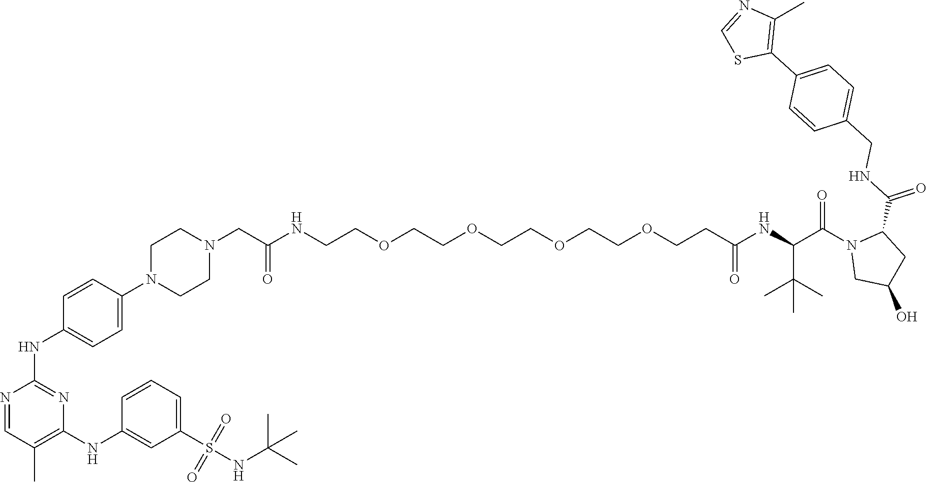 [SMILES: Cc1cnc(Nc2ccc(N3CCN(CC(=O)NCCOCCOCCOCCOCCC(=O)N[C@@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-20-(tert- butyl)-1-(4-(4-((4-((3-(N- (tert- butyl)sulfamoyl)phenyl)amino)- 5-methylpyrimidin-2- yl)amino)phenyl)piperazin-1- yl)-2,18-dioxo-6,9,12,15- tetraoxa-3,19- diazahenicosan-21-oyl)-4- hydroxy-N-(4-(4- methylthiazol-5- yl)benzyl)pyrrolidine-2- carboxamide |
| JA-223 | 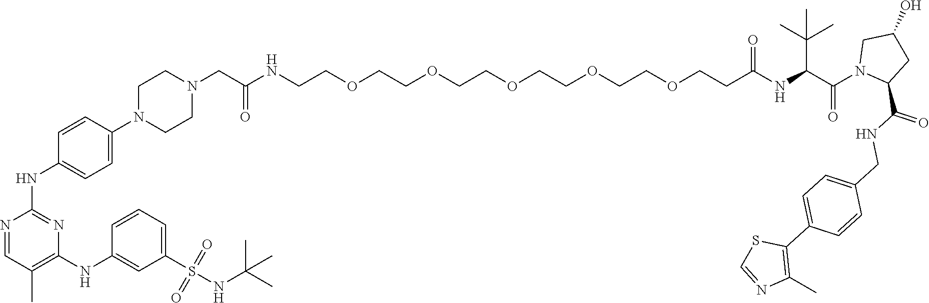 [SMILES: Cc1cnc(Nc2ccc(N3CCN(CC(=O)NCCOCCOCCOCCOCCOCCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-23-(tert- butyl)-1-(4-(4-((4-((3-(N- (tert- butyl)sulfamoyl)phenyl)amino)- 5-methylpyrimidin-2- yl)amino)phenyl)piperazin-1- yl)-2,21-dioxo-6,9,12,15,18- pentaoxa-3,22- diazatetracosan-24-oyl)-4- hydroxy-N-(4-(4- methylthiazol-5- yl)benzyl)pyrrolidine-2- carboxamide |
| JA-224 | 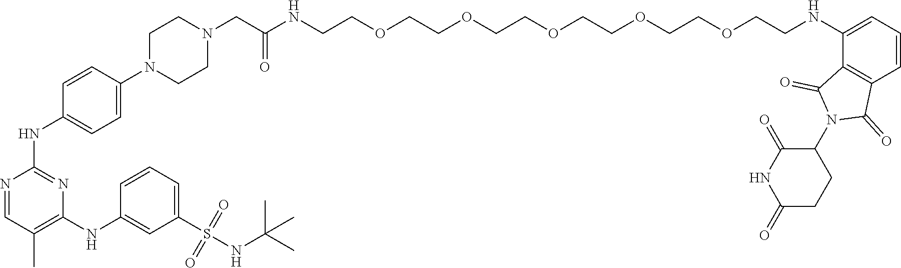 [SMILES: Cc1cnc(Nc2ccc(N3CCN(CC(=O)NCCOCCOCCOCCOCCOCCNc4cccc5c4C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(17-((2-(2,6- dioxopiperidin-3-yl)-1,3- dioxoisoindolin-4-yl)amino)- 3,6,9,12,15- pentaoxaheptadecyl)acetamide |
| JA-225 | 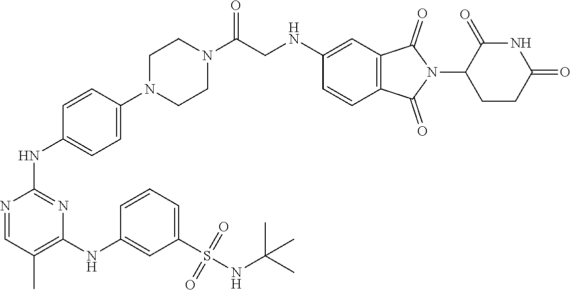 [SMILES: Cc1cnc(Nc2ccc(N3CCN(C(=O)CNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4- ((2-(2,6-dioxopiperidin-3-yl)- 1,3-dioxoisoindolin-5- yl)glycyl)piperazin-1- yl)phenyl)amino)-5- methylpyrimidin-4- yl)amino)benzenesulfonamide |
| JA-226 | 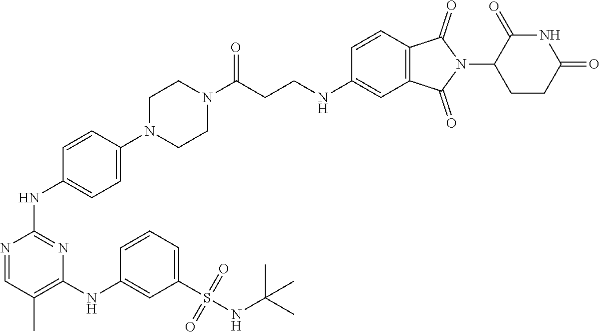 [SMILES: Cc1cnc(Nc2ccc(N3CCN(C(=O)CCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- ((2-(2,6-dioxopiperidin-3-yl)- 1,3-dioxoisoindolin-5- yl)amino)propanoyl) piperazin-1-yl)phenyl)amino)-5- methylpyrimidin-4- yl)amino)benzenesulfonamide |
| JA-227 | 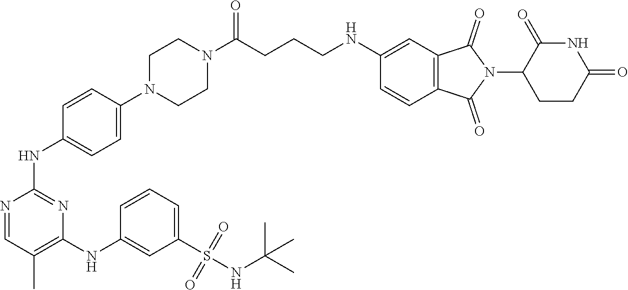 [SMILES: Cc1cnc(Nc2ccc(N3CCN(C(=O)CCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(4- ((2-(2,6-dioxopiperidin-3-yl)- 1,3-dioxoisoindolin-5- yl)amino)butanoyl) piperazin-1-yl)phenyl)amino)-5- methylpyrimidin-4- yl)amino)benzenesulfonamide |
| JA-228 | 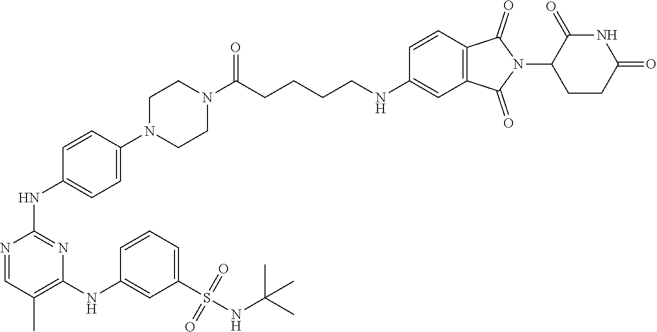 [SMILES: Cc1cnc(Nc2ccc(N3CCN(C(=O)CCCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(5- ((2-(2,6-dioxopiperidin-3-yl)- 1,3-dioxoisoindolin-5- yl)amino)pentanoyl) piperazin-1-yl)phenyl)amino)-5- methylpyrimidin-4- yl)amino)benzenesulfonamide |
| JA-229 | 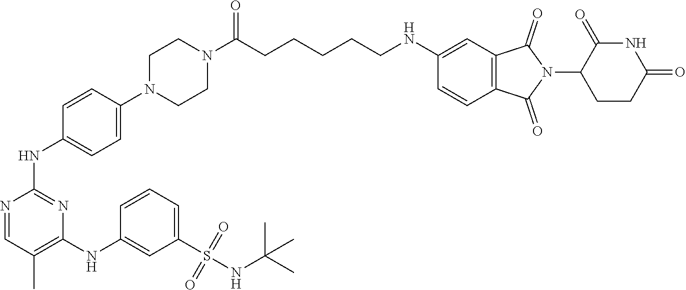 [SMILES: Cc1cnc(Nc2ccc(N3CCN(C(=O)CCCCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(6- ((2-(2,6-dioxopiperidin-3-yl)- 1,3-dioxoisoindolin-5- yl)amino)hexanoyl) piperazin-1-yl)phenyl)amino)-5- methylpyrimidin-4- yl)amino)benzenesulfonamide |
| JA-230 | 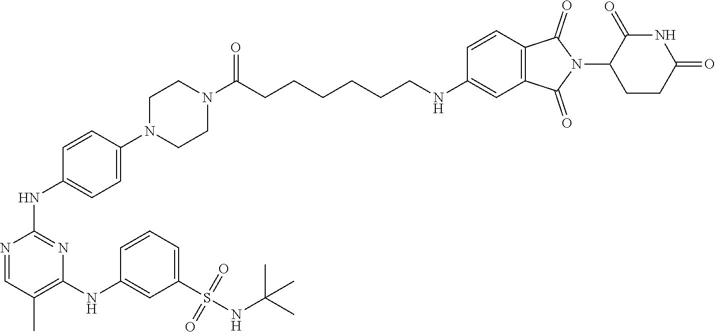 [SMILES: Cc1cnc(Nc2ccc(N3CCN(C(=O)CCCCCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(7- ((2-(2,6-dioxopiperidin-3-yl)- 1,3-dioxoisoindolin-5- yl)amino)heptanoyl) piperazin-1-yl)phenyl)amino)-5- methylpyrimidin-4- yl)amino)benzenesulfonamide |
| JA-231 | 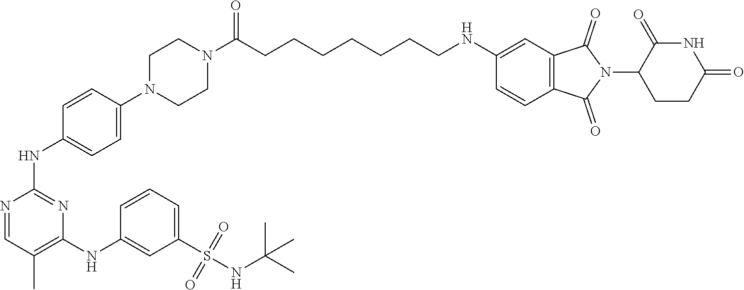 [SMILES: Cc1cnc(Nc2ccc(N3CCN(C(=O)CCCCCCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(8- ((2-(2,6-dioxopiperidin-3-yl)- 1,3-dioxoisoindolin-5- yl)amino)octanoyl) piperazin-1-yl)phenyl)amino)-5- methylpyrimidin-4- yl)amino)benzenesulfonamide |
| JA-232 | 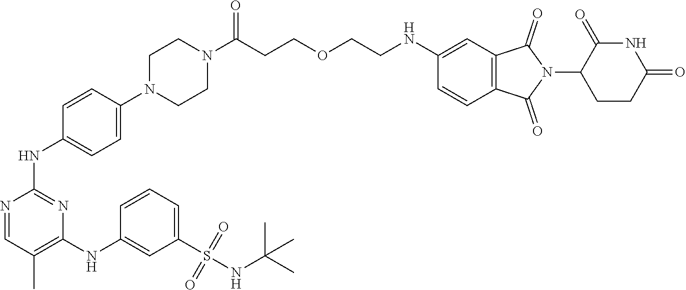 [SMILES: Cc1cnc(Nc2ccc(N3CCN(C(=O)CCOCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- (2-((2-(2,6-dioxopiperidin-3- yl)-1,3-dioxoisoindolin-5- yl)amino)ethoxy)propanoyl) piperazin-1-yl)phenyl)amino)- 5-methylpyrimidin-4- yl)amino)benzenesulfonamide |
| JA-233 | 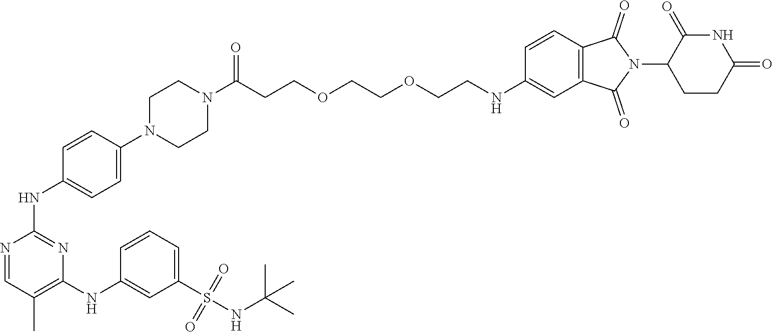 [SMILES: Cc1cnc(Nc2ccc(N3CCN(C(=O)CCOCCOCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- (2-(2-((2-(2,6- dioxopiperidin-3-yl)-1,3- dioxoisoindolin-5- yl)amino)ethoxy)ethoxy) propanoyl)piperazin-1- yl)phenyl)amino)-5- methylpyrimidin-4- yl)amino)benzenesulfonamide |
| JA-234 | 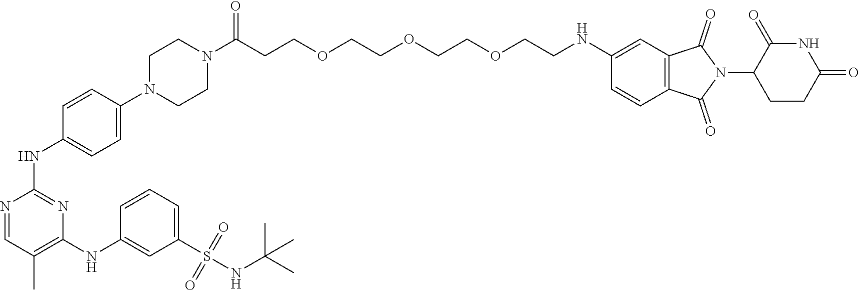 [SMILES: Cc1cnc(Nc2ccc(N3CCN(C(=O)CCOCCOCCOCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(3- (2-(2-(2-((2-(2,6- dioxopiperidin-3-yl)-1,3- dioxoisoindolin-5- yl)amino)ethoxy)ethoxy)ethoxy) propanoyl)piperazin-1- yl)phenyl)amino)-5- methylpyrimidin-4- yl)amino)benzenesulfonamide |
| JA-235 | 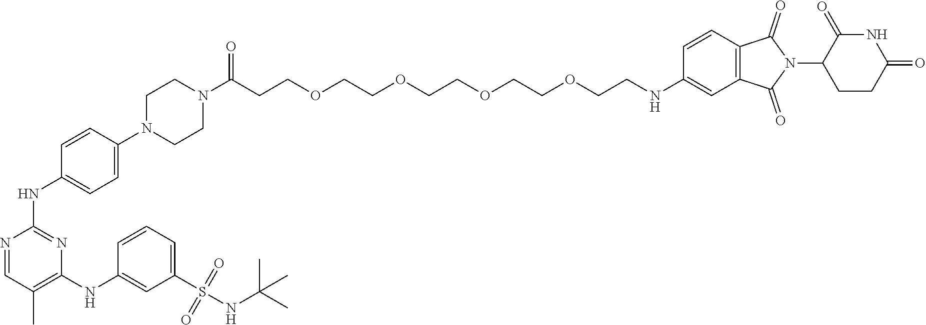 [SMILES: Cc1cnc(Nc2ccc(N3CCN(C(=O)CCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(1- ((2-(2,6-dioxopiperidin-3-yl)- 1,3-dioxoisoindolin-5- yl)amino)-3,6,9,12- tetraoxapentadecan-15- oyl)piperazin-1- yl)phenyl)amino)-5- methylpyrimidin-4- yl)amino)benzenesulfonamide |
| JA-236 | 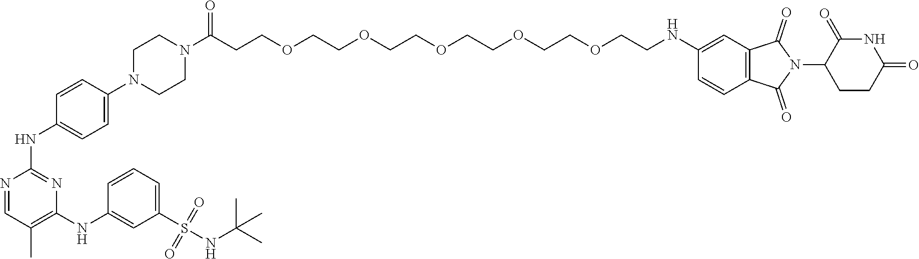 [SMILES: Cc1cnc(Nc2ccc(N3CCN(C(=O)CCOCCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1] | N-(tert-butyl)-3-((2-((4-(4-(1- ((2-(2,6-dioxopiperidin-3-yl)- 1,3-dioxoisoindolin-5- yl)amino)-3,6,9,12,15- pentaoxaoctadecan-18- oyl)piperazin-1- yl)phenyl)amino)-5- methylpyrimidin-4- yl)amino)benzenesulfonamide |
| JA-237 | 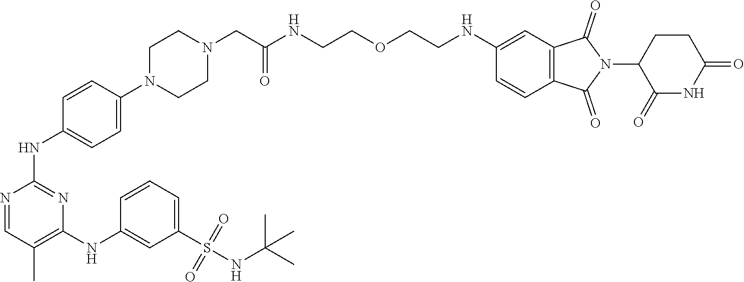 [SMILES: Cc1cnc(Nc2ccc(N3CCN(CC(=O)NCCOCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-(2-((2-(2,6- dioxopiperidin-3-yl)-1,3- dioxoisoindolin-5- yl)amino)ethoxy)ethyl) acetamide |
| JA-238 | 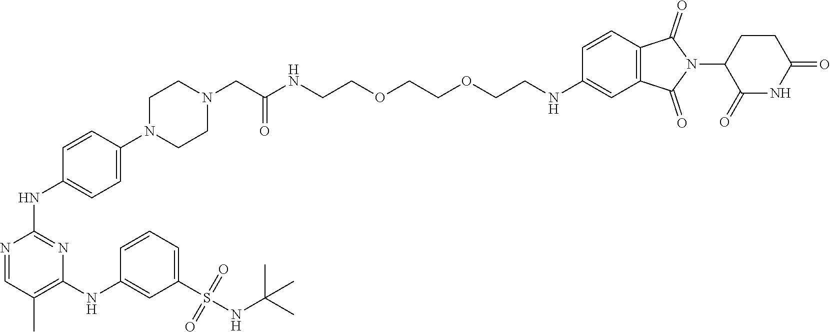 [SMILES: Cc1cnc(Nc2ccc(N3CCN(CC(=O)NCCOCCOCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-(2-(2-((2-(2,6- dioxopiperidin-3-yl)-1,3- dioxoisoindolin-5- yl)amino)ethoxy)ethoxy)ethyl) acetamide |
| JA-239 | 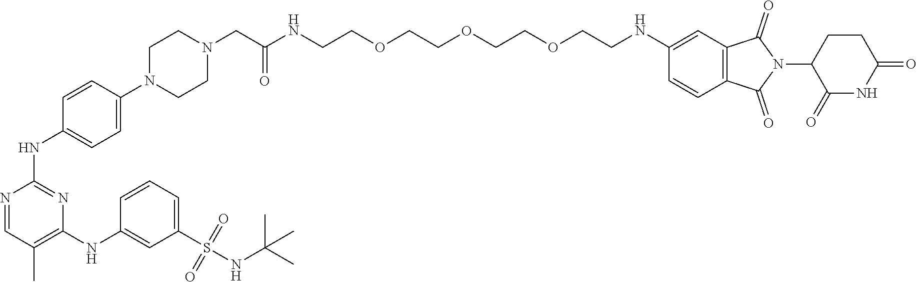 [SMILES: Cc1cnc(Nc2ccc(N3CCN(CC(=O)NCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-(2-(2-(2-((2-(2,6- dioxopiperidin-3-yl)-1,3- dioxoisoindolin-5- yl)amino)ethoxy)ethoxy)ethoxy) ethyl)acetamide |
| JA-240 | 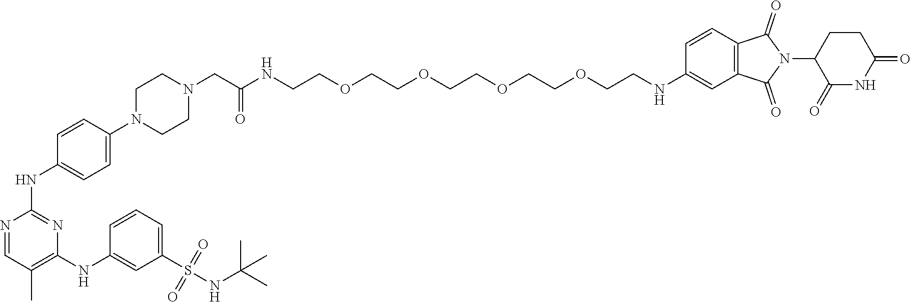 [SMILES: Cc1cnc(Nc2ccc(N3CCN(CC(=O)NCCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(14-((2-(2,6- dioxopiperidin-3-yl)-1,3- dioxoisoindolin-5-yl)amino)- 3,6,9,12- tetraoxatetradecyl)acetamide |
| JA-241 | 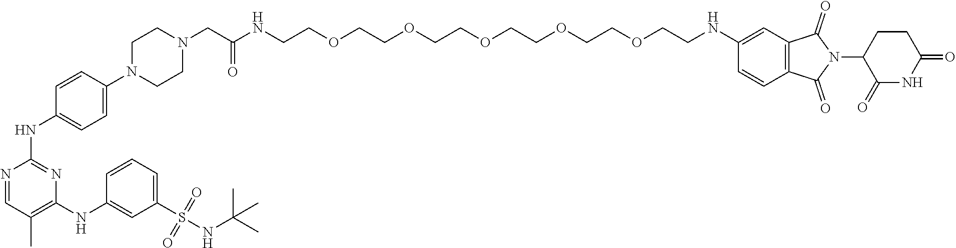 [SMILES: Cc1cnc(Nc2ccc(N3CCN(CC(=O)NCCOCCOCCOCCOCCOCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(17-((2-(2,6- dioxopiperidin-3-yl)-1,3- dioxoisoindolin-5-yl)amino)- 3,6,9,12,15- pentaoxaheptadecyl)acetamide |
| JA-242 | 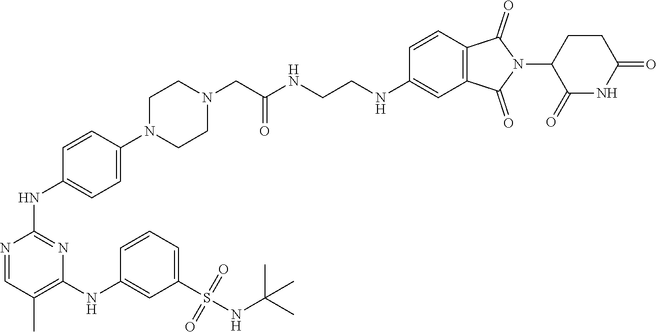 [SMILES: Cc1cnc(Nc2ccc(N3CCN(CC(=O)NCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(2-((2-(2,6- dioxopiperidin-3-yl)-1,3- dioxoisoindolin-5- yl)amino)ethyl)acetamide |
| JA-243 | 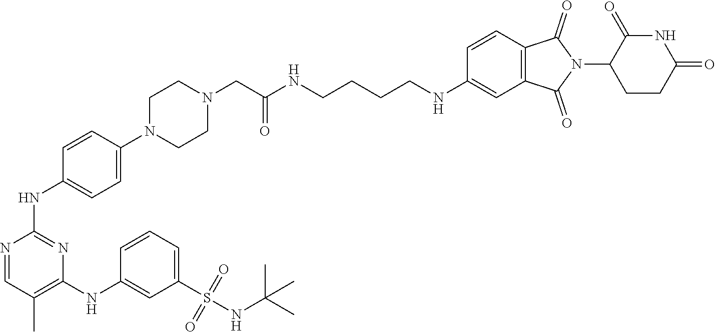 [SMILES: Cc1cnc(Nc2ccc(N3CCN(CC(=O)NCCCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(4-((2-(2,6- dioxopiperidin-3-yl)-1,3- dioxoisoindolin-5- yl)amino)butyl)acetamide |
| JA-244 | 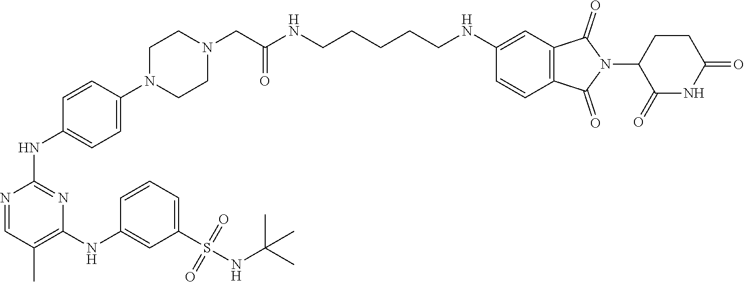 [SMILES: Cc1cnc(Nc2ccc(N3CCN(CC(=O)NCCCCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(5-((2-(2,6- dioxopiperidin-3-yl)-1,3- dioxoisoindolin-5- yl)amino)pentyl)acetamide |
| JA-245 | 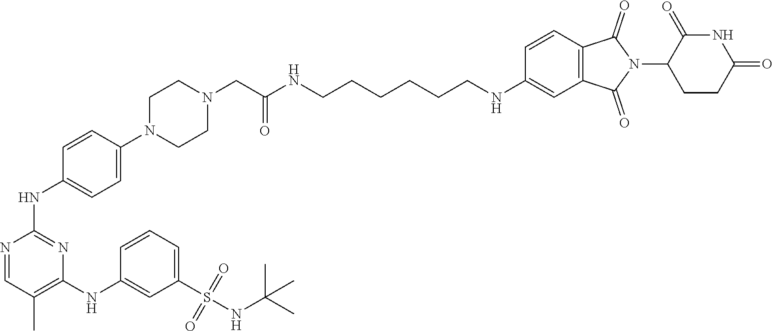 [SMILES: Cc1cnc(Nc2ccc(N3CCN(CC(=O)NCCCCCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(6-((2-(2,6- dioxopiperidin-3-yl)-1,3- dioxoisoindolin-5- yl)amino)hexyl)acetamide |
| JA-246 | 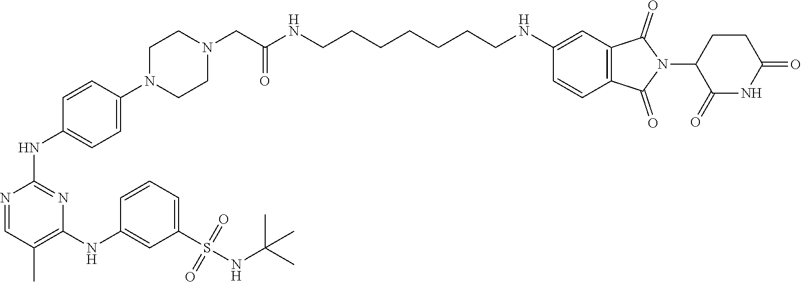 [SMILES: Cc1cnc(Nc2ccc(N3CCN(CC(=O)NCCCCCCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(7-((2-(2,6- dioxopiperidin-3-yl)-1,3- dioxoisoindolin-5- yl)amino)heptyl)acetamide |
| JA-247 | 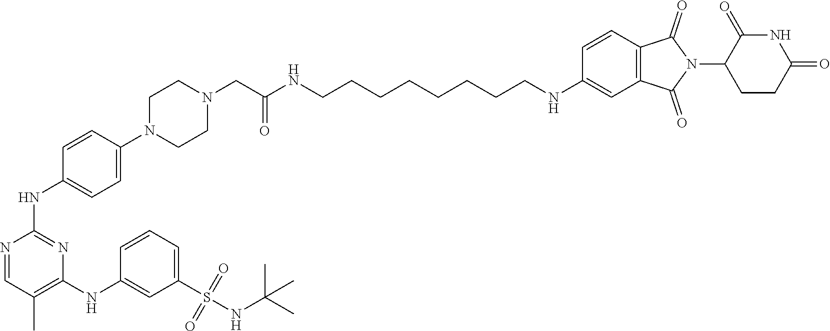 [SMILES: Cc1cnc(Nc2ccc(N3CCN(CC(=O)NCCCCCCCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(8-((2-(2,6- dioxopiperidin-3-yl)-1,3- dioxoisoindolin-5- yl)amino)octyl)acetamide |
| JA-248 | 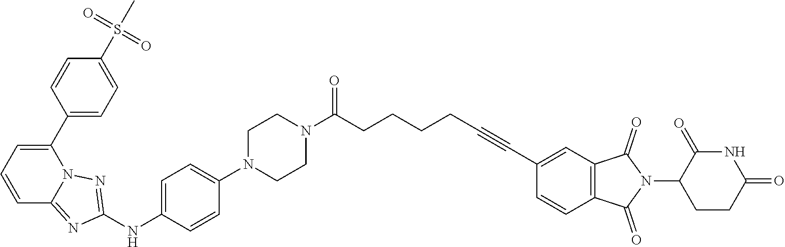 [SMILES: CS(=O)(=O)c1ccc(-c2cccc3nc(Nc4ccc(N5CCN(C(=O)CCCCC#Cc6ccc7c(c6)C(=O)N(C6CCC(=O)NC6=O)C7=O)CC5)cc4)nn23)cc1] | 2-(2,6-dioxopiperidin-3-yl)- 5-(7-(4-(4-((5-(4- (methylsulfonyl)phenyl)- [1,2,4]triazolo[1,5-a]pyridin- 2-yl)amino)phenyl)piperazin- 1-yl)-7-oxohept-1-yn-1- yl)isoindoline-1,3-dione |
| JA-249 | 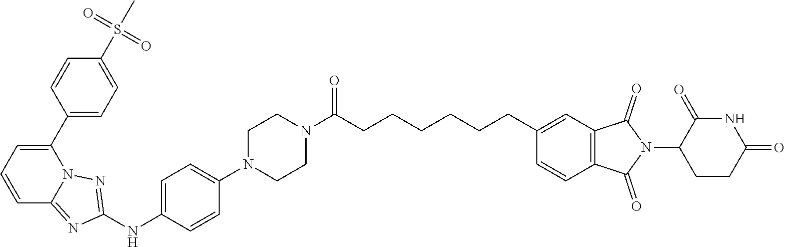 [SMILES: CS(=O)(=O)c1ccc(-c2cccc3nc(Nc4ccc(N5CCN(C(=O)CCCCCCc6ccc7c(c6)C(=O)N(C6CCC(=O)NC6=O)C7=O)CC5)cc4)nn23)cc1] | 2-(2,6-dioxopiperidin-3-yl)- 5-(7-(4-(4-((5-(4- (methylsulfonyl)phenyl)- [1,2,4]triazolo[1,5-a]pyridin- 2-yl)amino)phenyl)piperazin- 1-yl)-7- oxoheptyl)isoindoline-1,3- dione |
| JA-250 | 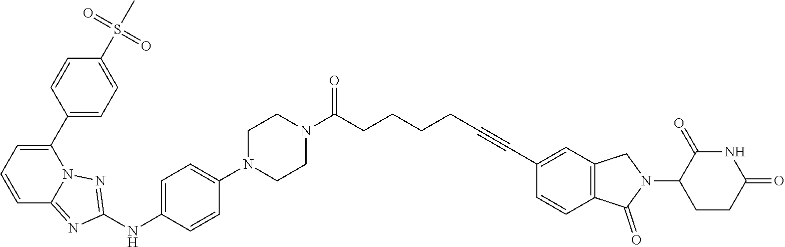 [SMILES: CS(=O)(=O)c1ccc(-c2cccc3nc(Nc4ccc(N5CCN(C(=O)CCCCC#Cc6ccc7c(c6)CN(C6CCC(=O)NC6=O)C7=O)CC5)cc4)nn23)cc1] | 3-(5-(7-(4-(4-((5-(4- (methylsulfonyl)phenyl)- [1,2,4]triazolo[1,5-a]pyridin- 2-yl)amino)phenyl)piperazin- 1-yl)-7-oxohept-1-yn-1-yl)-1- oxoisoindolin-2- yl)piperidine-2,6-dione |
| JA-251 | 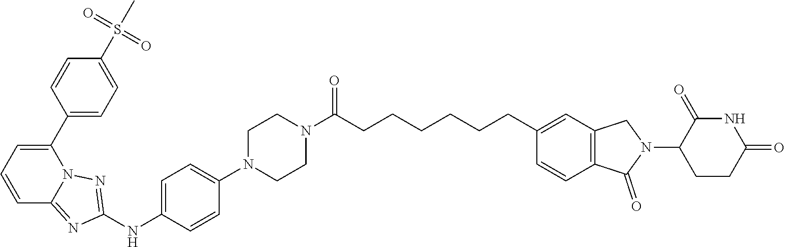 [SMILES: CS(=O)(=O)c1ccc(-c2cccc3nc(Nc4ccc(N5CCN(C(=O)CCCCCCc6ccc7c(c6)CN(C6CCC(=O)NC6=O)C7=O)CC5)cc4)nn23)cc1] | 3-(5-(7-(4-(4-((5-(4- (methylsulfonyl)phenyl)- [1,2,4]triazolo[1,5-a]pyridin- 2-yl)amino)phenyl)piperazin- 1-yl)-7-oxoheptyl)-1- oxoisoindolin-2- yl)piperidine-2,6-dione |
| JA-252 | 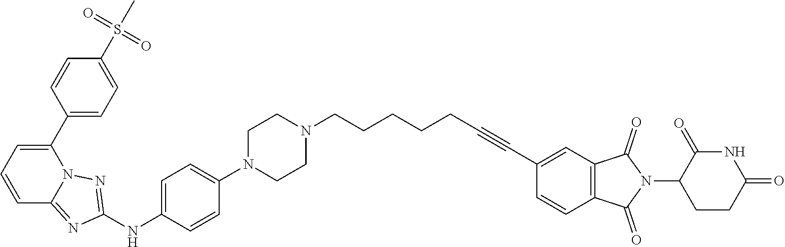 [SMILES: CS(=O)(=O)c1ccc(-c2cccc3nc(Nc4ccc(N5CCN(CCCCCC#Cc6ccc7c(c6)C(=O)N(C6CCC(=O)NC6=O)C7=O)CC5)cc4)nn23)cc1] | 2-(2,6-dioxopiperidin-3-yl)- 5-(7-(4-(4-((5-(4- (methylsulfonyl)phenyl)- [1,2,4]triazolo[1,5-a]pyridin- 2-yl)amino)phenyl)piperazin- 1-yl)hept-1-yn-1- yl)isoindoline-1,3-dione |
| JA-253 | 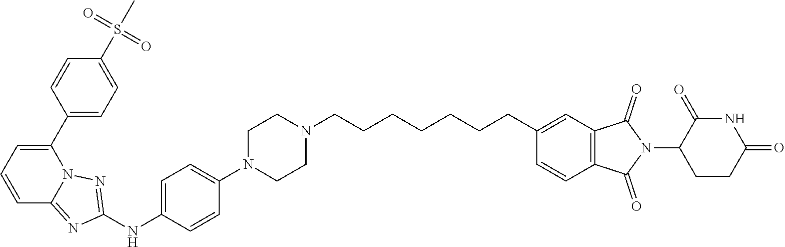 [SMILES: CS(=O)(=O)c1ccc(-c2cccc3nc(Nc4ccc(N5CCN(CCCCCCCc6ccc7c(c6)C(=O)N(C6CCC(=O)NC6=O)C7=O)CC5)cc4)nn23)cc1] | 2-(2,6-dioxopiperidin-3-yl)- 5-(7-(4-(4-((5-(4- (methylsulfonyl)phenyl)- [1,2,4]triazolo[1,5-a]pyridin- 2-yl)amino)phenyl)piperazin- 1-yl)heptyl)isoindoline-1,3- dione |
| JA-254 | 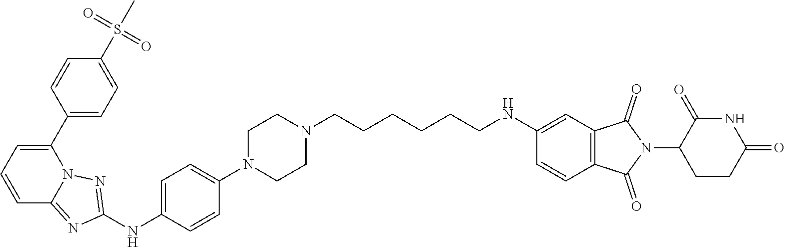 [SMILES: CS(=O)(=O)c1ccc(-c2cccc3nc(Nc4ccc(N5CCN(CCCCCCNc6ccc7c(c6)C(=O)N(C6CCC(=O)NC6=O)C7=O)CC5)cc4)nn23)cc1] | 2-(2,6-dioxopiperidin-3-yl)- 5-((6-(4-(4-((5-(4- (methylsulfonyl)phenyl)- [1,2,4]triazolo[1,5-a]pyridin- 2-yl)amino)phenyl)piperazin- 1- yl)hexyl)amino)isoindoline- 1,3-dione |
| JA-255 | 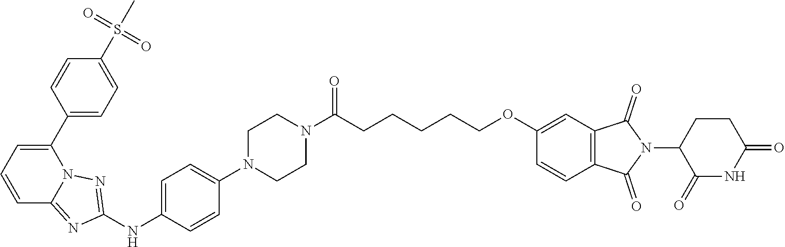 [SMILES: CS(=O)(=O)c1ccc(-c2cccc3nc(Nc4ccc(N5CCN(C(=O)CCCCCOc6ccc7c(c6)C(=O)N(C6CCC(=O)NC6=O)C7=O)CC5)cc4)nn23)cc1] | 2-(2,6-dioxopiperidin-3-yl)- 5-((6-(4-(4-((5-(4- (methylsulfonyl)phenyl)- [1,2,4]triazolo[1,5-a]pyridin- 2-yl)amino)phenyl)piperazin- 1-yl)-6- oxohexyl)oxy)isoindoline- 1,3-dione |
| JA-256 | 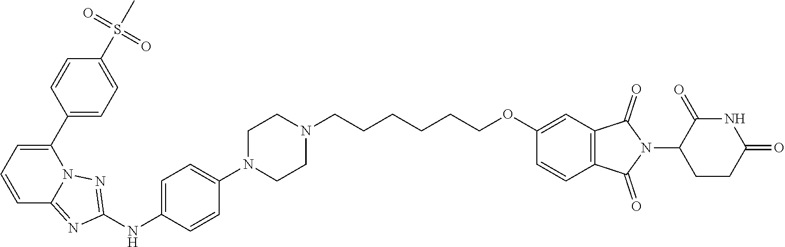 [SMILES: CS(=O)(=O)c1ccc(-c2cccc3nc(Nc4ccc(N5CCN(CCCCCCOc6ccc7c(c6)C(=O)N(C6CCC(=O)NC6=O)C7=O)CC5)cc4)nn23)cc1] | 2-(2,6-dioxopiperidin-3-yl)- 5-((6-(4-(4-((5-(4- (methylsulfonyl)phenyl)- [1,2,4]triazolo[1,5-a]pyridin- 2-yl)amino)phenyl)piperazin- 1-yl)hexyl)oxy)isodoline- 1,3-dione |
| JA-257 | 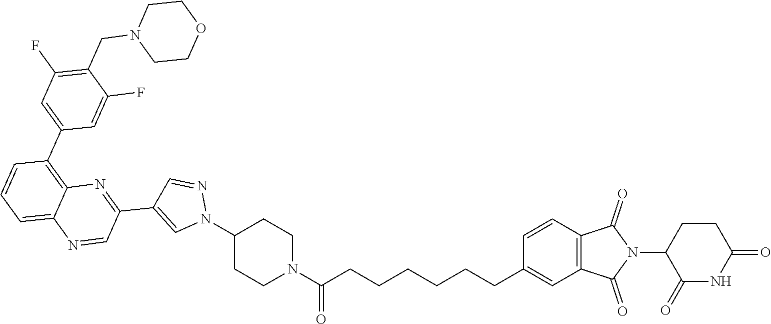 [SMILES: O=C1CCC(N2C(=O)c3ccc(CCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-(7-(4-(4-(8-(3,5-difluoro-4- (morpholinomethyl)phenyl) quinoxalin-2-yl)-1H-pyrazol- 1-yl)piperidin-1-yl)-7- oxoheptyl)-2-(2,6- dioxopiperidin-3- yl)isoindoline-1,3-dione |
| JA-258 | 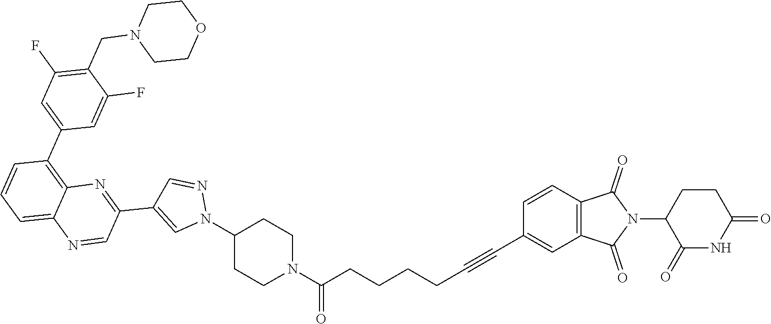 [SMILES: O=C1CCC(N2C(=O)c3ccc(C#CCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-(7-(4-(4-(8-(3,5-difluoro-4- (morpholinomethyl)phenyl) quinoxalin-2-yl)-1H-pyrazol- 1-yl)piperidin-1-yl)-7- oxohept-1-yn-1-yl)-2-(2,6- dioxopiperidin-3- yl)isoindoline-1,3-dione |
| JA-259 | 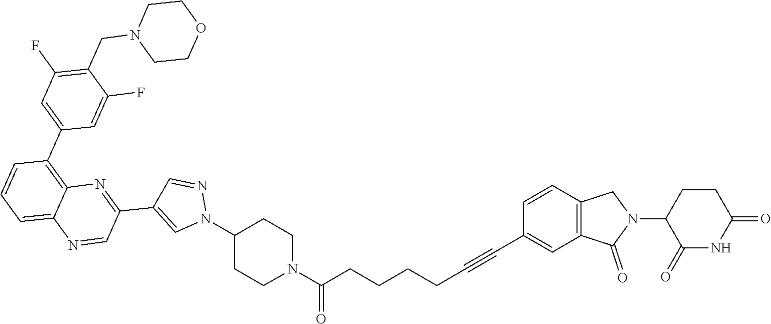 [SMILES: O=C1CCC(N2Cc3ccc(C#CCCCCC(=O)N4CCC(n5cc(-c6cnc7cccc(-c8cc(F)c(CN9CCOCC9)c(F)c8)c7n6)cn5)CC4)cc3C2=O)C(=O)N1] | 3-(6-(7-(4-(4-(8-(3,5- difluoro-4- (morpholinomethyl)phenyl) quinoxalin-2-yl)-1H-pyrazol- 1-yl)piperidin-1-yl)-7- oxohept-1-yn-1-yl)-1- oxoisoindolin-2- yl)piperidine-2,6-dione |
| JA-260 | 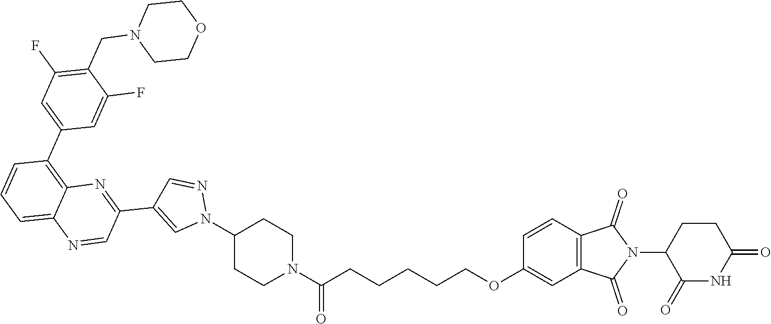 [SMILES: O=C1CCC(N2C(=O)c3ccc(OCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 5-((6-(4-(4-(8-(3,5-difluoro- 4- (morpholinomethyl)phenyl) quinoxalin-2-yl)-1H-pyrazol- 1-yl)piperidin-1-yl)-6- oxohexyl)oxy)-2-(2,6- dioxopiperidin-3- yl)isoindoline-1,3-dione |
| JA-261 | 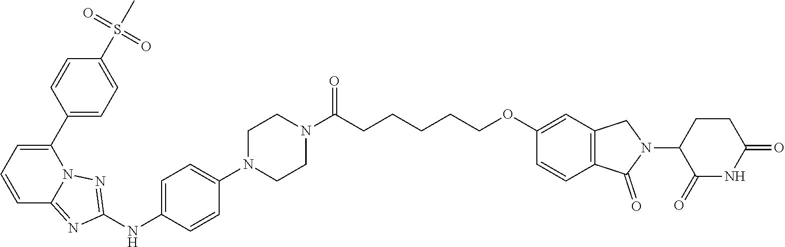 [SMILES: CS(=O)(=O)c1ccc(-c2cccc3nc(Nc4ccc(N5CCN(C(=O)CCCCCOc6ccc7c(c6)CN(C6CCC(=O)NC6=O)C7=O)CC5)cc4)nn23)cc1] | 3-(5-((6-(4-(4-((5-(4- (methylsulfonyl)phenyl)- [1,2,4]triazolo[1,5-a]pyridin- 2-yl)amino)phenyl)piperazin- 1-yl)-6-oxohexyl)oxy)-1- oxoisoindolin-2- yl)piperidine-2,6-dione |
| JA-262 | 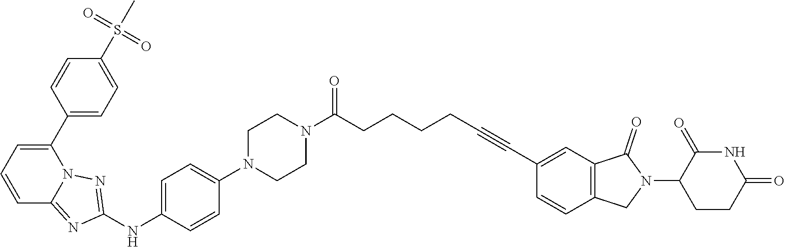 [SMILES: CS(=O)(=O)c1ccc(-c2cccc3nc(Nc4ccc(N5CCN(C(=O)CCCCC#Cc6ccc7c(c6)C(=O)N(C6CCC(=O)NC6=O)C7)CC5)cc4)nn23)cc1] | 3-(6-(7-(4-(4-((5-(4- (methylsulfonyl)phenyl)- [1,2,4]triazolo[1,5-a]pyridin- 2-yl)amino)phenyl)piperazin- 1-yl)-7-oxohept-1-yn-1-yl)-1- oxoisoindolin-2- yl)piperidine-2,6-dione |
| JA-263 | 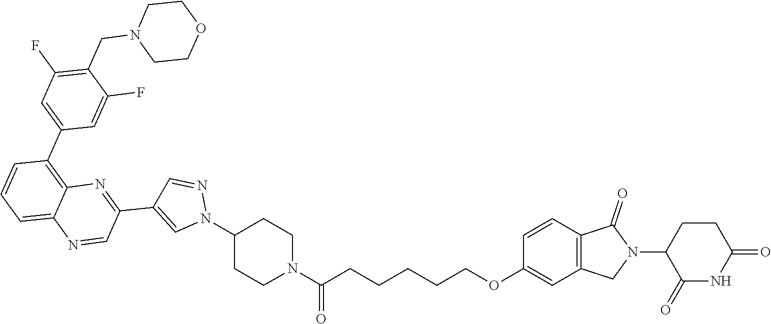 [SMILES: O=C1CCC(N2Cc3cc(OCCCCCC(=O)N4CCC(n5cc(-c6cnc7cccc(-c8cc(F)c(CN9CCOCC9)c(F)c8)c7n6)cn5)CC4)ccc3C2=O)C(=O)N1] | 3-(5-((6-(4-(4-(8-(3,5- difluoro-4- (morpholinomethyl)phenyl) quinoxalin-2-yl)-1H-pyrazol- 1-yl)piperidin-1-yl)-6- oxohexyl)oxy)-1- oxoisoindolin-2- yl)piperidine-2,6-dione |
| JA-264 | 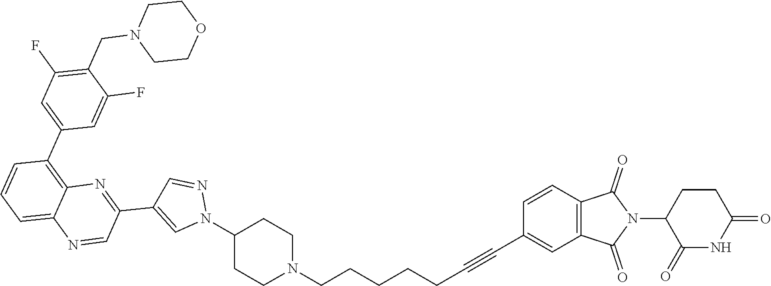 [SMILES: O=C1CCC(N2C(=O)c3ccc(C#CCCCCCN4CCC(n5cc(-c6cnc7cccc(-c8cc(F)c(CN9CCOCC9)c(F)c8)c7n6)cn5)CC4)cc3C2=O)C(=O)N1] | 5-(7-(4-(4-(8-(3,5-difluoro-4- (morpholinomethyl)phenyl) quinoxalin-2-yl)-1H-pyrazol- 1-yl)piperidin-1-yl)hept-1- yn-1-yl)-2-(2,6- dioxopiperidin-3- yl)isoindoline-1,3-dione |
| JA-265 | 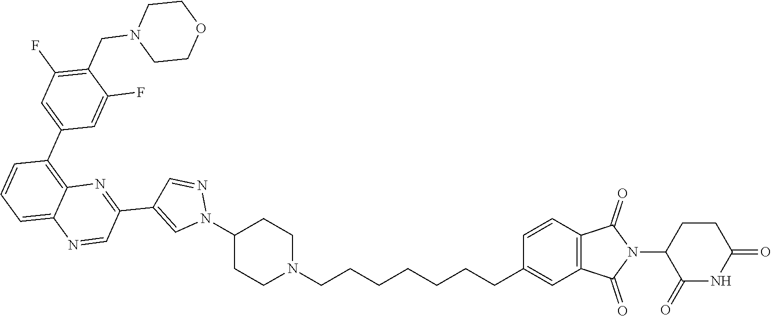 [SMILES: O=C1CCC(N2C(=O)c3ccc(CCCCCCCN4CCC(n5cc(-c6cnc7cccc(-c8cc(F)c(CN9CCOCC9)c(F)c8)c7n6)cn5)CC4)cc3C2=O)C(=O)N1] | 5-(7-(4-(4-(8-(3,5-difluoro-4- (morpholinomethyl)phenyl) quinoxalin-2-yl)-1H-pyrazol- 1-yl)piperidin-1-yl)heptyl)-2- (2,6-dioxopiperidin-3- yl)isoindoline-1,3-dione |
| JA-266 | 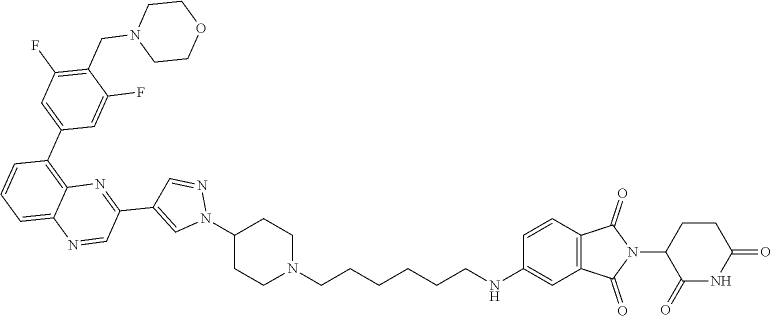 [SMILES: O=C1CCC(N2C(=O)c3ccc(NCCCCCCN4CCC(n5cc(-c6cnc7cccc(-c8cc(F)c(CN9CCOCC9)c(F)c8)c7n6)cn5)CC4)cc3C2=O)C(=O)N1] | 5-((6-(4-(4-(8-(3,5-difluoro- 4- (morpholinomethyl)phenyl) quinoxalin-2-yl)-1H-pyrazol- 1-yl)piperidin-1- yl)hexyl)amino)-2-(2,6- dioxopiperidin-3- yl)isoindoline-1,3-dione |
| JA-267 | 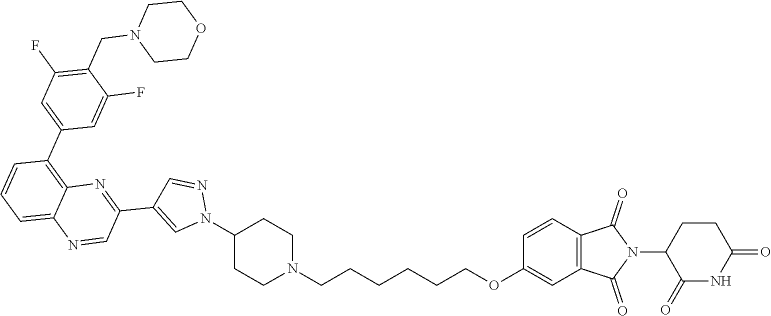 [SMILES: O=C1CCC(N2C(=O)c3ccc(OCCCCCCN4CCC(n5cc(-c6cnc7cccc(-c8cc(F)c(CN9CCOCC9)c(F)c8)c7n6)cn5)CC4)cc3C2=O)C(=O)N1] | 5-((6-(4-(4-(8-(3,5-difluoro- 4- (morpholinomethyl)phenyl) quinoxalin-2-yl)-1H-pyrazol- 1-yl)piperidin-1- yl)hexyl)oxy)-2-(2,6- dioxopiperidin-3- yl)isoindoline-1,3-dione |
| JA-268 | 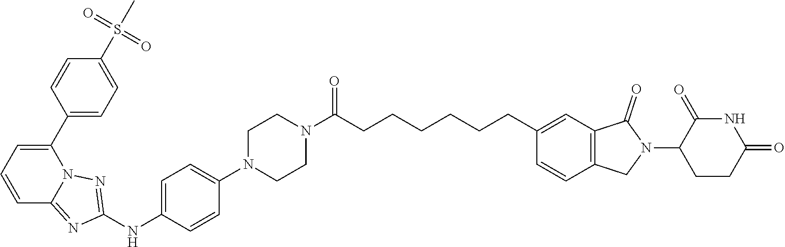 [SMILES: CS(=O)(=O)c1ccc(-c2cccc3nc(Nc4ccc(N5CCN(C(=O)CCCCCCc6ccc7c(c6)C(=O)N(C6CCC(=O)NC6=O)C7)CC5)cc4)nn23)cc1] | 3-(6-(7-(4-(4-((5-(4- (methylsulfonyl)phenyl)- [1,2,4]triazolo[1,5-a]pyridin- 2-yl)amino)phenyl)piperazin- 1-yl)-7-oxoheptyl)-1- oxoisoindolin-2- yl)piperidine-2,6-dione |
| JA-269 | 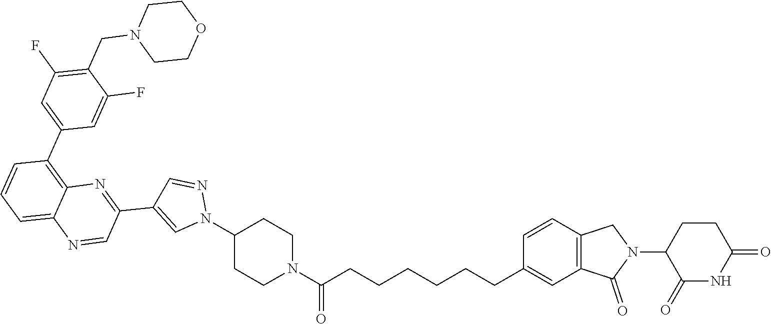 [SMILES: O=C1CCC(N2Cc3ccc(CCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 3-(6-(7-(4-(4-(8-(3,5- difluoro-4- (morpholinomethyl)phenyl) quinoxalin-2-yl)-1H-pyrazol- 1-yl)piperidin-1-yl)-7- oxoheptyl)-1-oxoisoindolin- 2-yl)piperidine-2,6-dione |
| JA-270 | 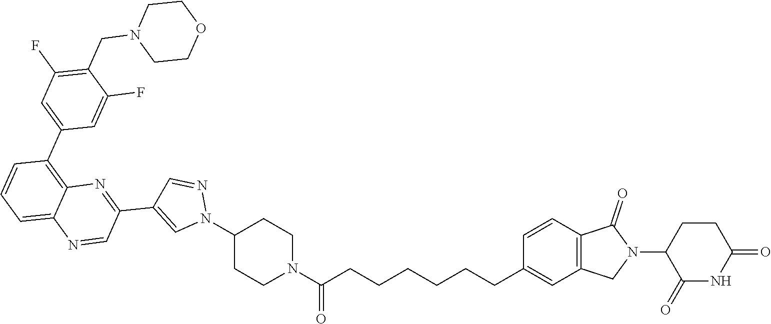 [SMILES: O=C1CCC(N2Cc3cc(CCCCCCC(=O)N4CCC(n5cc(-c6cnc7cccc(-c8cc(F)c(CN9CCOCC9)c(F)c8)c7n6)cn5)CC4)ccc3C2=O)C(=O)N1] | 3-(5-(7-(4-(4-(8-(3,5- difluoro-4- (morpholinomethyl)phenyl) quinoxalin-2-yl)-1H-pyrazol- 1-yl)piperidin-1-yl)-7- oxoheptyl)-1-oxoisoindolin- 2-yl)piperidine-2,6-dione |
| JA-271 | 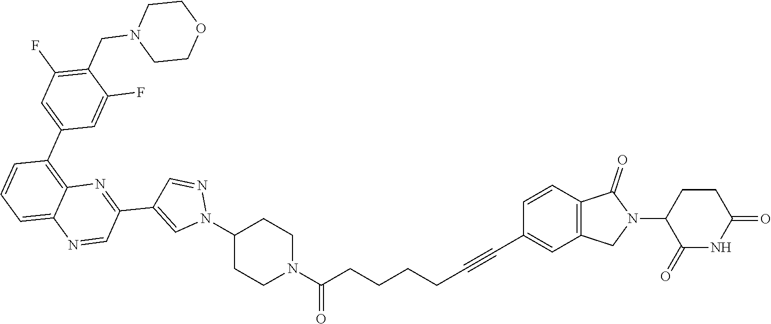 [SMILES: O=C1CCC(N2Cc3cc(C#CCCCCC(=O)N4CCC(n5cc(-c6cnc7cccc(-c8cc(F)c(CN9CCOCC9)c(F)c8)c7n6)cn5)CC4)ccc3C2=O)C(=O)N1] | 3-(5-(7-(4-(4-(8-(3,5- difluoro-4- (morpholinomethyl)phenyl) quinoxalin-2-yl)-1H-pyrazol- 1-yl)piperidin-1-yl)-7- oxohept-1-yn-1-yl)-1- oxoisoindolin-2- yl)piperidine-2,6-dione |
| JA-272 | 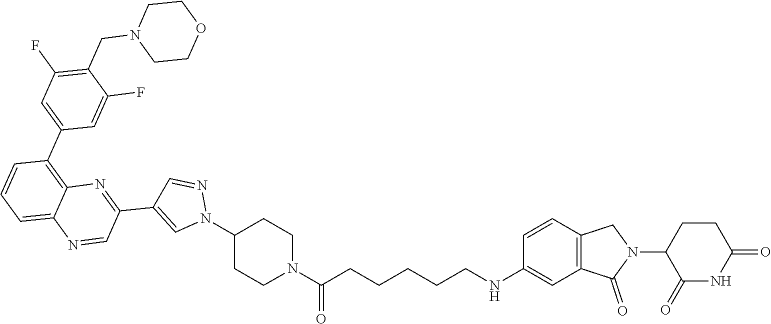 [SMILES: O=C1CCC(N2Cc3ccc(NCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 3-(6-((6-(4-(4-(8-(3,5- difluoro-4- (morpholinomethyl)phenyl) quinoxalin-2-yl)-1H-pyrazol- 1-yl)piperidin-1-yl)-6- oxohexyl)amino)-1- oxoisoindolin-2- yl)piperidine-2,6-dione |
| JA-273 | 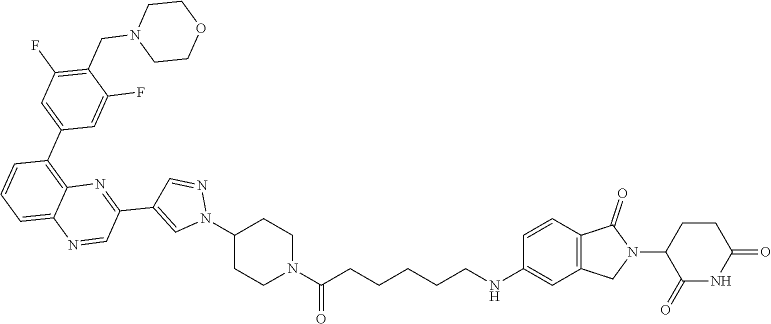 [SMILES: O=C1CCC(N2Cc3cc(NCCCCCC(=O)N4CCC(n5cc(-c6cnc7cccc(-c8cc(F)c(CN9CCOCC9)c(F)c8)c7n6)cn5)CC4)ccc3C2=O)C(=O)N1] | 3-(5-((6-(4-(4-(8-(3,5- difluoro-4- (morpholinomethyl)phenyl) quinoxalin-2-yl)-1H-pyrazol- 1-yl)piperidin-1-yl)-6- oxohexyl)amino)-1- oxoisoindolin-2- yl)piperidine-2,6-dione |
| JA-274 | 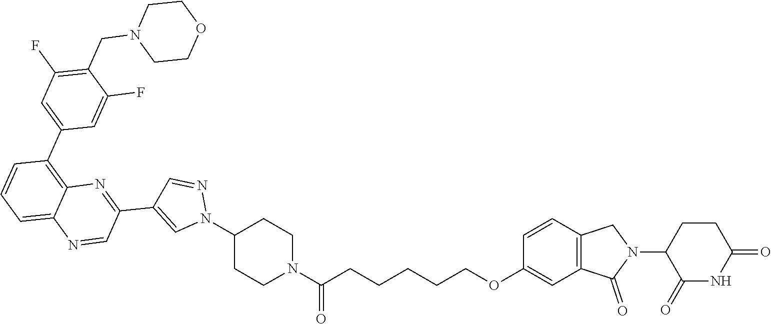 [SMILES: O=C1CCC(N2Cc3ccc(OCCCCCC(=O)N4CCC(n5cc(-c6cnc7cccc(-c8cc(F)c(CN9CCOCC9)c(F)c8)c7n6)cn5)CC4)cc3C2=O)C(=O)N1] | 3-(6-((6-(4-(4-(8-(3,5- difluoro-4- (morpholinomethyl)phenyl) quinoxalin-2-yl)-1H-pyrazol- 1-yl)piperidin-1-yl)-6- oxohexyl)oxy)-1- oxoisoindolin-2- yl)piperidine-2,6-dione |
| JA-275 | 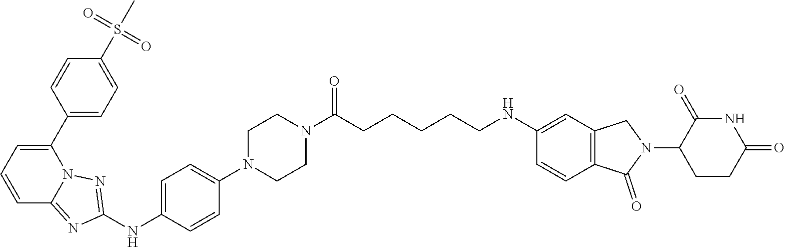 [SMILES: CS(=O)(=O)c1ccc(-c2cccc3nc(Nc4ccc(N5CCN(C(=O)CCCCCNc6ccc7c(c6)CN(C6CCC(=O)NC6=O)C7=O)CC5)cc4)nn23)cc1] | 3-(5-((6-(4-(4-((5-(4- (methylsulfonyl)phenyl)- [1,2,4]triazolo[1,5-a]pyridin- 2-yl)amino)phenyl)piperazin- 1-yl)-6-oxohexyl)amino)-1- oxoisoindolin-2- yl)piperidine-2,6-dione |
| JA-276 | 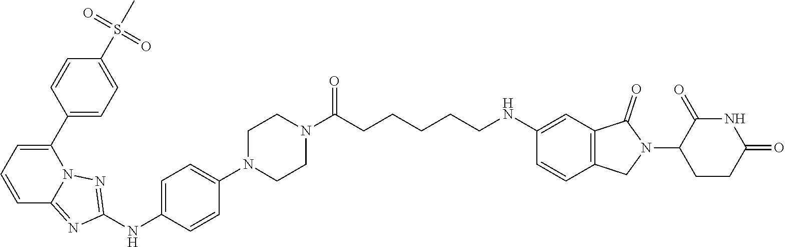 [SMILES: CS(=O)(=O)c1ccc(-c2cccc3nc(Nc4ccc(N5CCN(C(=O)CCCCCNc6ccc7c(c6)C(=O)N(C6CCC(=O)NC6=O)C7)CC5)cc4)nn23)cc1] | 3-(6-((6-(4-(4-((5-(4- (methylsulfonyl)phenyl)- [1,2,4]triazolo[1,5-a]pyridin- 2-yl)amino)phenyl)piperazin- 1-yl)-6-oxohexyl)amino)-1- oxoisoindolin-2- yl)piperidine-2,6-dione |
| JA-277 | 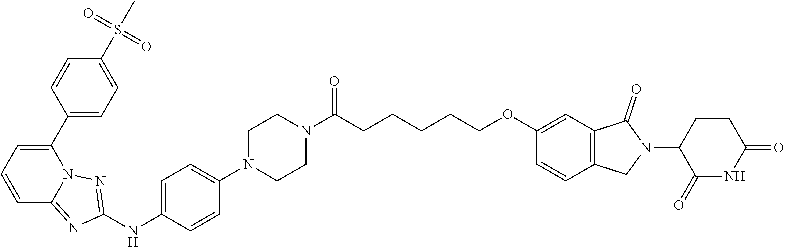 [SMILES: CS(=O)(=O)c1ccc(-c2cccc3nc(Nc4ccc(N5CCN(C(=O)CCCCCOc6ccc7c(c6)C(=O)N(C6CCC(=O)NC6=O)C7)CC5)cc4)nn23)cc1] | 3-(6-((6-(4-(4-((5-(4- (methylsulfonyl)phenyl)- [1,2,4]triazolo[1,5-a]pyridin- 2-yl)amino)phenyl)piperazin- 1-yl)-6-oxohexyl)oxy)-1- oxoisoindolin-2- yl)piperidine-2,6-dione |
| JA-278 | 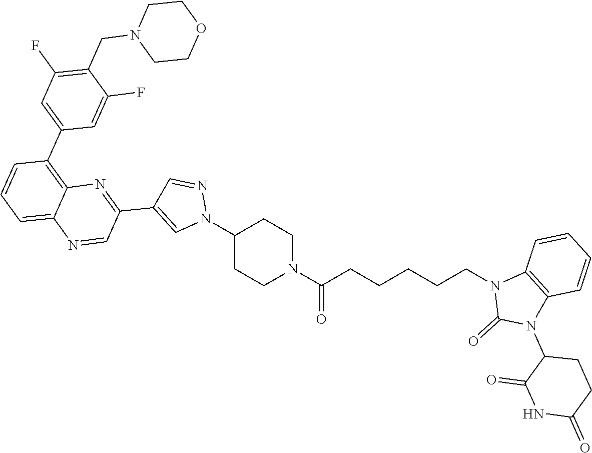 [SMILES: O=C1CCC(n2c(=O)n(CCCCCC(=O)N3CCC(n4cc(-c5cnc6cccc(-c7cc(F)c(CN8CCOCC8)c(F)c7)c6n5)cn4)CC3)c3ccccc32)C(=O)N1] | 3-(3-(6-(4-(4-(8-(3,5- difluoro-4- (morpholinomethyl)phenyl) quinoxalin-2-yl)-1H-pyrazol- 1-yl)piperidin-1-yl)-6- oxohexyl)-2-oxo-2,3- dihydro-1H- benzo[d]imidazol-1- yl)piperidine-2,6-dione |
| JA-279 | 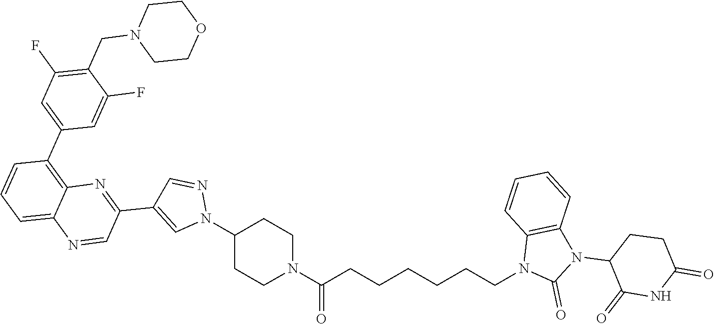 [SMILES: O=C1CCC(n2c(=O)n(CCCCCCC(=O)N3CCC(n4cc(-c5cnc6cccc(-c7cc(F)c(CN8CCOCC8)c(F)c7)c6n5)cn4)CC3)c3ccccc32)C(=O)N1] | 3-(3-(7-(4-(4-(8-(3,5- difluoro-4- (morpholinomethyl)phenyl) quinoxalin-2-yl)-1H-pyrazol- 1-yl)piperidin-1-yl)-7- oxoheptyl)-2-oxo-2,3- dihydro-1H- benzo[d]imidazol-1- yl)piperidine-2,6-dione |
| JA-280 | 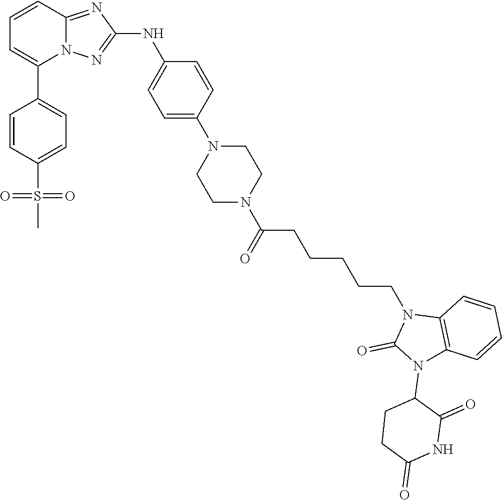 [SMILES: CS(=O)(=O)c1ccc(-c2cccc3nc(Nc4ccc(N5CCN(C(=O)CCCCCn6c(=O)n(C7CCC(=O)NC7=O)c7ccccc76)CC5)cc4)nn23)cc1] | 3-(3-(6-(4-(4-((5-(4- (methylsulfonyl)phenyl)- [1,2,4]triazolo[1,5-a]pyridin- 2-yl)amino)phenyl)piperazin- 1-yl)-6-oxohexyl)-2-oxo-2,3- dihydro-1H- benzo[d]imidazol-1- yl)piperidine-2,6-dione |
| JA-281 | 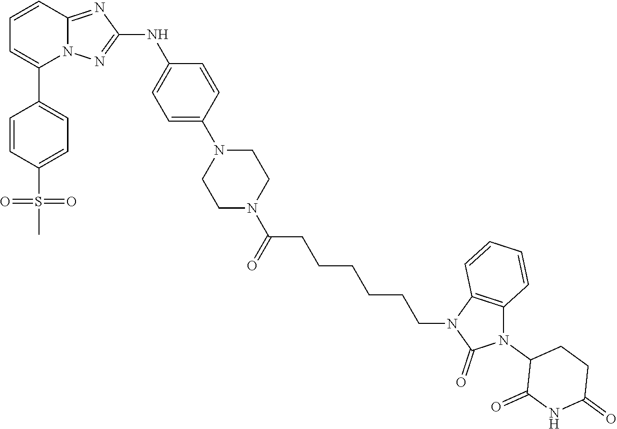 [SMILES: CS(=O)(=O)c1ccc(-c2cccc3nc(Nc4ccc(N5CCN(C(=O)CCCCCCn6c(=O)n(C7CCC(=O)NC7=O)c7ccccc76)CC5)cc4)nn23)cc1] | 3-(3-(7-(4-(4-((5-(4- (methylsulfonyl)phenyl)- [1,2,4]triazolo[1,5-a]pyridin- 2-yl)amino)phenyl)piperazin- 1-yl)-7-oxoheptyl)-2-oxo- 2,3-dihydro-1H- benzo[d]imidazol-1- yl)piperidine-2,6-dione |
| JA-282 | 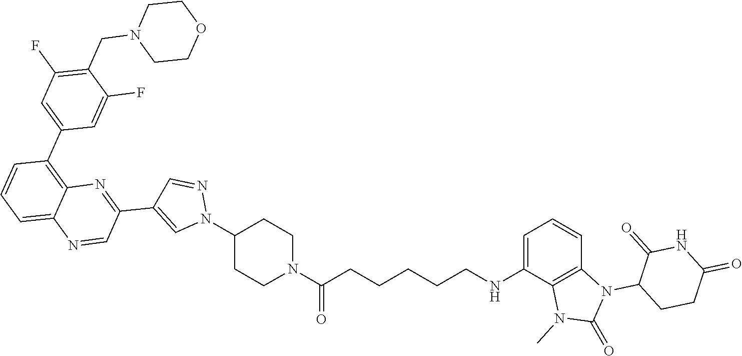 [SMILES: Cn1c(=O)n(C2CCC(=O)NC2=O)c2cccc(NCCCCCC(=O)N3CCC(n4cc(-c5cnc6cccc(-c7cc(F)c(CN8CCOCC8)c(F)c7)c6n5)cn4)CC3)c21] | 3-(4-((6-(4-(4-(8-(3,5- difluoro-4- (morpholinomethyl)phenyl) quinoxalin-2-yl)-1H-pyrazol- 1-yl)piperidin-1-yl)-6- oxohexyl)amino)-3-methyl- 2-oxo-2,3-dihydro-1H- benzo[d]imidazol-1- yl)piperidine-2,6-dione |
| JA-283 | 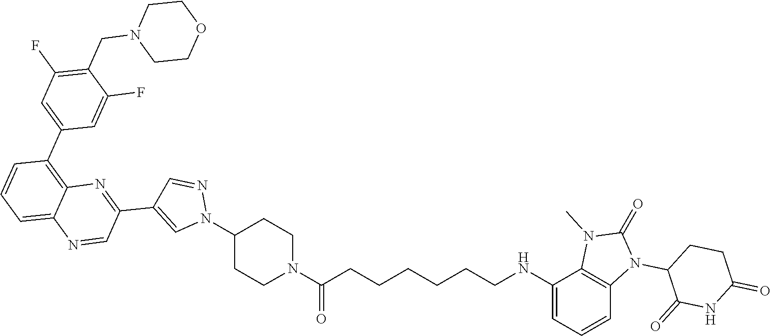 [SMILES: Cn1c(=O)n(C2CCC(=O)NC2=O)c2cccc(NCCCCCCC(=O)N3CCC(n4cc(-c5cnc6cccc(-c7cc(F)c(CN8CCOCC8)c(F)c7)c6n5)cn4)CC3)c21] | 3-(4-((7-(4-(4-(8-(3,5- difluoro-4- (morpholinomethyl)phenyl) quinoxalin-2-yl)-1H-pyrazol- 1-yl)piperidin-1-yl)-7- oxoheptyl)amino)-3-methyl- 2-oxo-2,3-dihydro-1H- benzo[d]imidazol-1- yl)piperidine-2,6-dione |
| JA-284 | 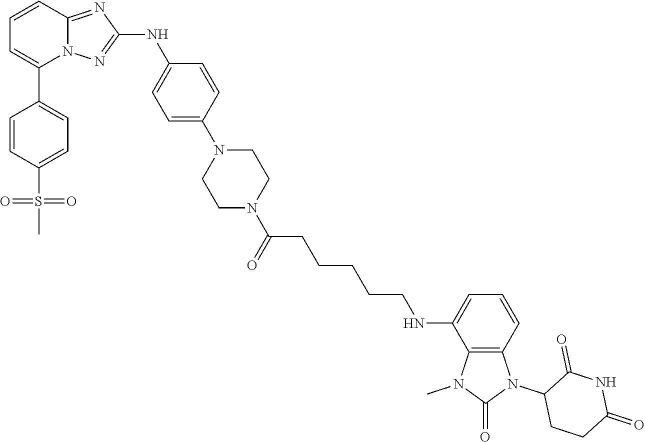 [SMILES: Cn1c(=O)n(C2CCC(=O)NC2=O)c2cccc(NCCCCCC(=O)N3CCN(c4ccc(Nc5nc6cccc(-c7ccc(S(C)(=O)=O)cc7)n6n5)cc4)CC3)c21] | 3-(3-methyl-4-((6-(4-(4-((5- (4-(methylsulfonyl)phenyl)- [1,2,4]triazolo[1,5-a]pyridin- 2-yl)amino)phenyl)piperazin- 1-yl)-6-oxohexyl)amino)-2- oxo-2,3-dihydro-1H- benzo[d]imidazol-1- yl)piperidine-2,6-dione |
| JA-285 | 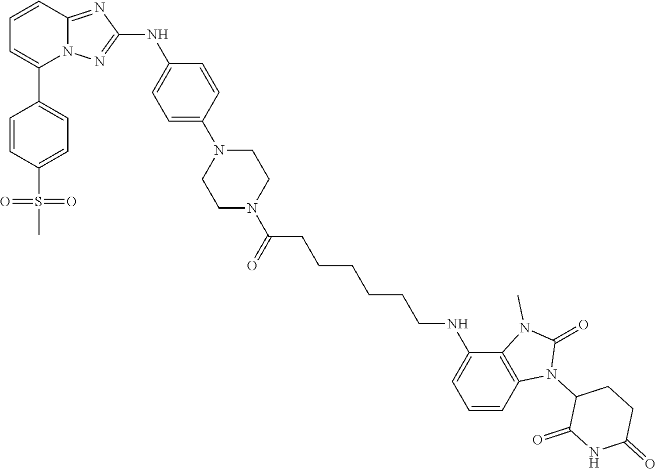 [SMILES: Cn1c(=O)n(C2CCC(=O)NC2=O)c2cccc(NCCCCCCC(=O)N3CCN(c4ccc(Nc5nc6cccc(-c7ccc(S(C)(=O)=O)cc7)n6n5)cc4)CC3)c21] | 3-(3-methyl-4-((7-(4-(4-((5- (4-(methylsulfonyl)phenyl)- [1,2,4]triazolo[1,5-a]pyridin- 2-yl)amino)phenyl)piperazin- 1-yl)-7-oxoheptyl)amino)-2- oxo-2,3-dihydro-1H- benzo[d]imidazol-1- yl)piperidine-2,6-dione |
| JA-286 | 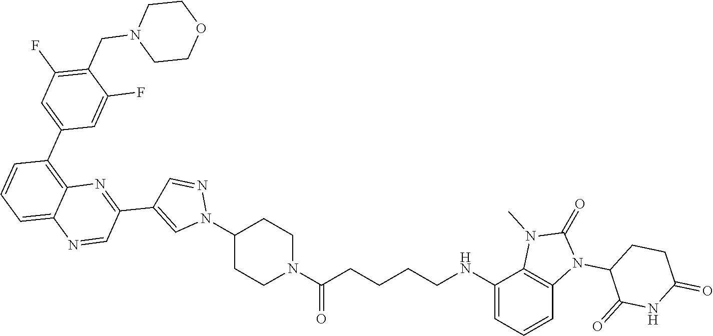 [SMILES: Cn1c(=O)n(C2CCC(=O)NC2=O)c2cccc(NCCCCC(=O)N3CCC(n4cc(-c5cnc6cccc(-c7cc(F)c(CN8CCOCC8)c(F)c7)c6n5)cn4)CC3)c21] | 3-(4-((5-(4-(4-(8-(3,5- difluoro-4- (morpholinomethyl)phenyl) quinoxalin-2-yl)-1H-pyrazol- 1-yl)piperidin-1-yl)-5- oxopentyl)amino)-3-methyl- 2-oxo-2,3-dihydro-1H- benzo[d]imidazol-1- yl)piperidine-2,6-dione |
| JA-287 | 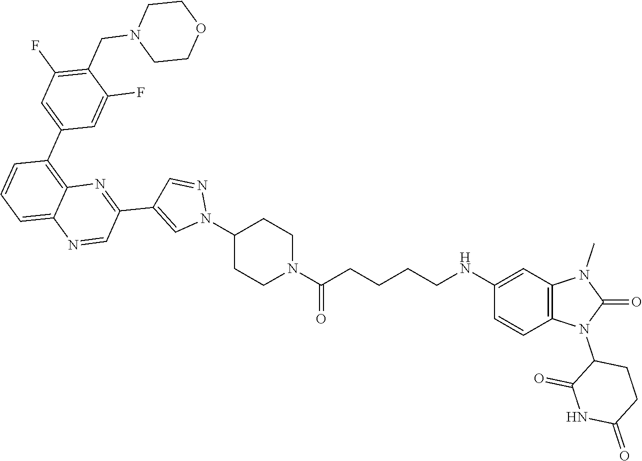 [SMILES: Cn1c(=O)n(C2CCC(=O)NC2=O)c2ccc(NCCCCC(=O)N3CCC(n4cc(-c5cnc6cccc(-c7cc(F)c(CN8CCOCC8)c(F)c7)c6n5)cn4)CC3)cc21] | 3-(5-((5-(4-(4-(8-(3,5- difluoro-4- (morpholinomethyl)phenyl) quinoxalin-2-yl)-1H-pyrazol- 1-yl)piperidin-1-yl)-5- oxopentyl)amino)-3-methyl- 2-oxo-2,3-dihydro-1H- benzo[d]imidazol-1- yl)piperidine-2,6-dione |
| JA-288 | 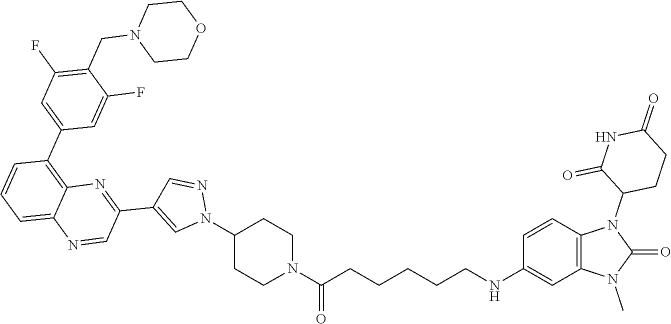 [SMILES: Cn1c(=O)n(C2CCC(=O)NC2=O)c2ccc(NCCCCCC(=O)N3CCC(n4cc(-c5cnc6cccc(-c7cc(F)c(CN8CCOCC8)c(F)c7)c6n5)cn4)CC3)cc21] | 3-(5-((6-(4-(4-(8-(3,5- difluoro-4- (morpholinomethyl)phenyl) quinoxalin-2-yl)-1H-pyrazol- 1-yl)piperidin-1-yl)-6- oxohexyl)amino)-3-methyl- 2-oxo-2,3-dihydro-1H- benzo[d]imidazol-1- yl)piperidine-2,6-dione |
| JA-289 | 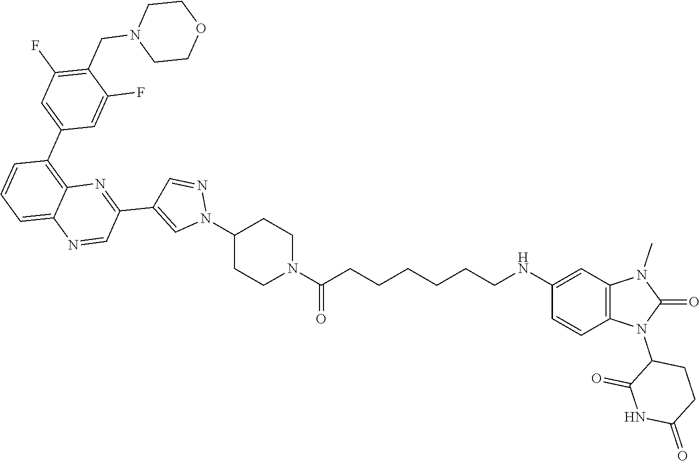 [SMILES: Cn1c(=O)n(C2CCC(=O)NC2=O)c2ccc(NCCCCCCC(=O)N3CCC(n4cc(-c5cnc6cccc(-c7cc(F)c(CN8CCOCC8)c(F)c7)c6n5)cn4)CC3)cc21] | 3-(5-((7-(4-(4-(8-(3,5- difluoro-4- (morpholinomethyl)phenyl) quinoxalin-2-yl)-1H-pyrazol- 1-yl)piperidin-1-yl)-7- oxoheptyl)amino)-3-methyl- 2-oxo-2,3-dihydro-1H- benzo[d]imidazol-1- yl)piperidine-2,6-dione |
| JA-290 | 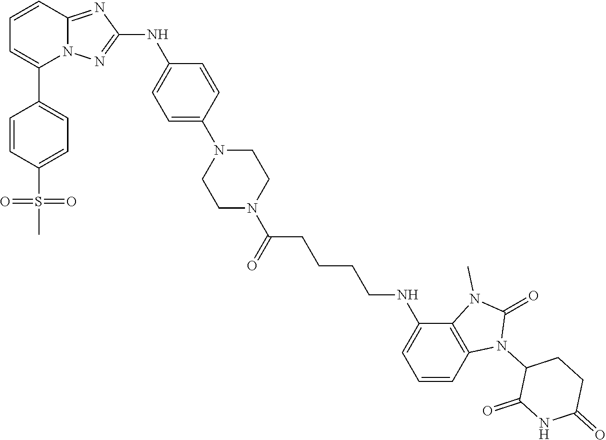 [SMILES: Cn1c(=O)n(C2CCC(=O)NC2=O)c2cccc(NCCCCC(=O)N3CCN(c4ccc(Nc5nc6cccc(-c7ccc(S(C)(=O)=O)cc7)n6n5)cc4)CC3)c21] | 3-(3-methyl-4-((5-(4-(4-((5- (4-(methylsulfonyl)phenyl)- [1,2,4]triazolo[1,5-a]pyridin- 2-yl)amino)phenyl)piperazin- 1-yl)-5-oxopentyl)amino)-2- oxo-2,3-dihydro-1H- benzo[d]imidazol-1- yl)piperidine-2,6-dione |
| JA-291 | 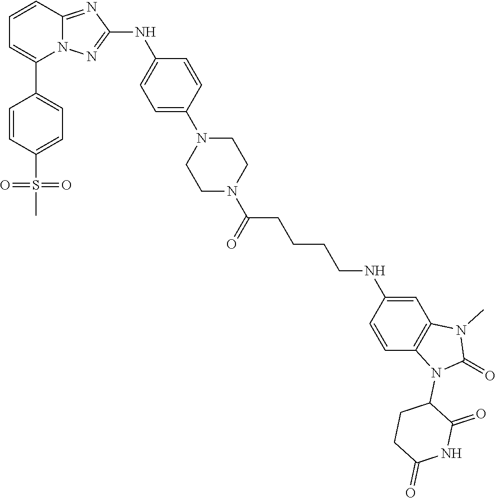 [SMILES: Cn1c(=O)n(C2CCC(=O)NC2=O)c2ccc(NCCCCC(=O)N3CCN(c4ccc(Nc5nc6cccc(-c7ccc(S(C)(=O)=O)cc7)n6n5)cc4)CC3)cc21] | 3-(3-methyl-5-((5-(4-(4-((5- (4-(methylsulfonyl)phenyl)- [1,2,4]triazolo[1,5-a]pyridin- 2-yl)amino)phenyl)piperazin- 1-yl)-5-oxopentyl)amino)-2- oxo-2,3-dihydro-1H- benzo[d]imidazol-1- yl)piperidine-2,6-dione |
| JA-292 | 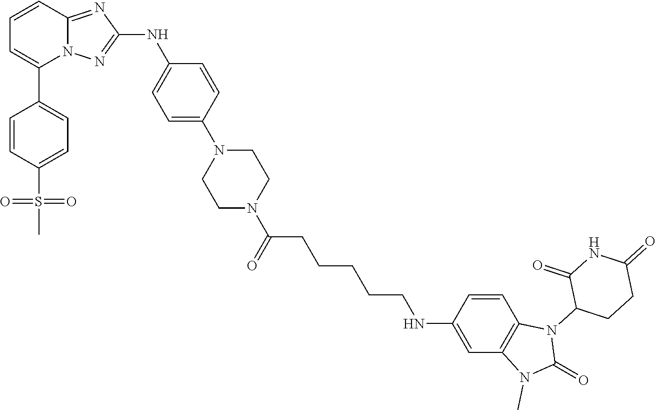 [SMILES: Cn1c(=O)n(C2CCC(=O)NC2=O)c2ccc(NCCCCCC(=O)N3CCN(c4ccc(Nc5nc6cccc(-c7ccc(S(C)(=O)=O)cc7)n6n5)cc4)CC3)cc21] | 3-(3-methyl-5-((6-(4-(4-((5- (4-(methylsulfonyl)phenyl)- [1,2,4]triazolo[1,5-a]pyridin- 2-yl)amino)phenyl)piperazin- 1-yl)-6-oxohexyl)amino)-2- oxo-2,3-dihydro-1H- benzo[d]imidazol-1- yl)piperidine-2,6-dione |
| JA-293 | 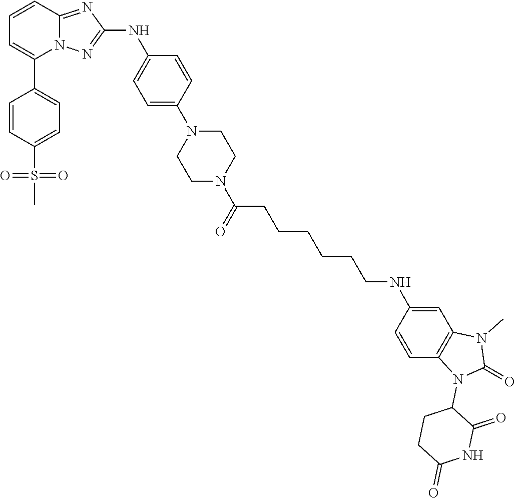 [SMILES: Cn1c(=O)n(C2CCC(=O)NC2=O)c2ccc(NCCCCCCC(=O)N3CCN(c4ccc(Nc5nc6cccc(-c7ccc(S(C)(=O)=O)cc7)n6n5)cc4)CC3)cc21] | 3-(3-methyl-5-((7-(4-(4-((5- (4-(methylsulfonyl)phenyl)- [1,2,4]triazolo[1,5-a]pyridin- 2-yl)amino)phenyl)piperazin- 1-yl)-7-oxoheptyl)amino)-2- oxo-2,3-dihydro-1H- benzo[d]imidazol-1- yl)piperidine-2,6-dione |
| JA-294 | 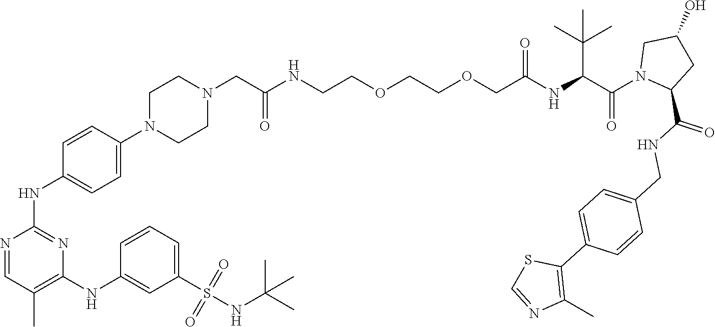 [SMILES: Cc1cnc(Nc2ccc(N3CCN(CC(=O)NCCOCCOCC(=O)N[C@H](C(=O)N4C[C@H](O)C[C@H]4C(=O)NCc4ccc(-c5scnc5C)cc4)C(C)(C)C)CC3)cc2)nc1Nc1cccc(S(=O)(=O)NC(C)(C)C)c1] | (2S,4R)-1-((S)-2-(tert-butyl)- 14-(4-(4-((4-((3-(N-(tert- butyl)sulfamoyl)phenyl)amino)- 5-methylpyrimidin-2- yl)amino)phenyl)piperazin-1- yl)-4,13-dioxo-6,9-dioxa- 3,12-diazatetradecanoyl)-4- hydroxy-N-(4-(4- methylthiazol-5- yl)benzyl)pyrrolidine-2- carboxamide |
| JA-295 | 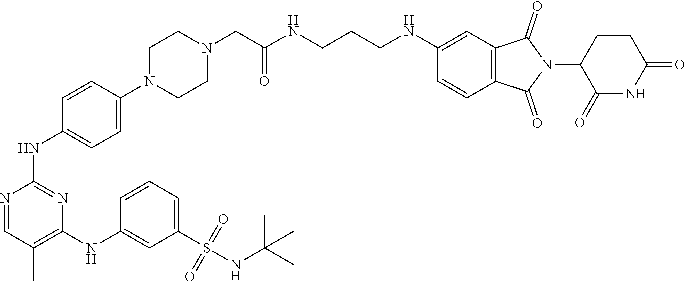 [SMILES: Cc1cnc(Nc2ccc(N3CCN(CC(=O)NCCCNc4ccc5c(c4)C(=O)N(C4CCC(=O)NC4=O)C5=O)CC3)cc2)nc1Nc1cccc(S(=O)(=O)NC(C)(C)C)c1] | 2-(4-(4-((4-((3-(N-(tert- butyl)sulfamoyl)phenyl)amino)- 5-methylpyrimidin-2- yl)amino)phenyl)piperazin-1- yl)-N-(3-((2-(2,6- dioxopiperidin-3-yl)-1,3- dioxoisoindolin-5- yl)amino)propyl)acetamide |

As used herein, in case of discrepancy between the structure and chemical name provided for a particular compound, the structure shall control.

### Example 382. Selected JAK Degraders Concentration-Dependently Reduced JAK2 and JAK1 Protein Levels in HEL Cells (FIG. 1)

HEL cells harboring JAK2-V617F mutation were treated with compounds at indicated concentrations for 24 hours. Data showed that JAK2-V617F and JAK1 proteins levels were reduced in a concentration-dependent manner. The concentrations required to reduce JAK2-V617 by 50% (DC₅₀) were below 60 nM for compound JA-213.

### Example 383. Selected JAK Degraders Concentration-Dependently Reduced JAK1 Protein Levels in RS4;11 Cells (FIG. 2)

RS4;11 cells were treated with compounds at indicated concentrations for 24 hours. Data showed that JAK1 proteins levels were reduced in a concentration-dependent manner. The concentrations required to reduce JAK1 by 50% (DC₅₀) were below 5 nM and 35 nM for compound JA-189 and JA213, respectively.

### Example 384. Selected JAK Degraders Suppressed Viability of Some Leukemia Cells (FIG. 3 & Table. 3)

MV4;11, RS4;11, Kasumi-1, HEL and other cells were treated with NVP-BSK805, TG101209 or selected degraders for 3 days at indicated concentrations following a 3-fold serial dilution. Data indicated that JAK degraders significantly and selectively suppressed viability of some leukemia cells while their warhead, NVP-BSK805 or TG101209 has none or less effect.

### Example 385. Selected JAK Degraders Caused Cell Viability Inhibition is CRBN Dependent (FIG. 4)

MV4;11, RS4;11, Kasumi-1 and HEL were treated with NVP-BSK805, TG101209 or selected degraders for 3 days at indicated concentrations in the presence or absence of pomalidomide at 10 μM. Data showed that JAK degraders caused cell viability inhibition was completely compromised by high concentration of pomalidomide, which competes JAK degraders off from Cereblon (CRBN) E3 ligase. Taking together, data indicated that JAK degraders caused cell viability inhibition is CRBN dependent.

### Example 386. Selected JAK Degraders Concentration-Dependently Reduced GSPT1 Protein Levels in RS4;11 Cells (FIG. 5)

RS4;11 cells were treated with compounds at indicated concentrations for 16 hours. Data showed that GSPT1 proteins levels were reduced in a concentration-dependent manner. The concentrations required to reduce GSPT1 by 50% (DC₅₀) were below 2 nM for compound JA-189 and 10 nM JA213.

### Example 387. Non-Cancerous Human Cells are Resistant to Selected JAK Degraders (FIG. 6)

Immortalized human lung fibroblast IMR-90 and keratinocyte HACAT cells were treated with JA-189 or JA-213 for 3 days at indicated concentrations following a 3-fold serial dilution. Data indicate that these non-cancerous human cells were not sensitive to these compounds, suggesting potential therapeutic windows.

Materials and Methods:

General Chemistry Methods:

All chemicals and reagents were purchased from commercial suppliers and used without further purification. LCMS spectra for all compounds were acquired using a Shimadzu LC-MS 2020 system or a Waters UPLC-MS H class system. The Shimadzu LC-MS 2020 system comprising a pump (LC-20AD) with degasser (DGU-20A3), an autosampler (SIL-20AHT), a column oven (CTO-20A) (set at 40° C., unless otherwise indicated), a photo-diode array (PDA) (SPD-M20A) detector, an evaporative light-scattering (ELSD) (Alltech 3300ELSD) detector. Chromatography was performed on a Shimadzu SunFire C18 (5 μm 50*4.6 mm) with water containing 0.1% formic acid as solvent A and acetonitrile containing 0.1% formic acid as solvent B at a flow rate of 2.0 ml/min. Flow from the column was split to a MS spectrometer. The MS detector was configured with an electrospray ionization source. Nitrogen was used as the nebulizer gas. Data acquisition was performed with a Labsolution data system. The Waters UPLC-MS H class system comprising a pump (Quaternary Solvent Manager) with degasser, an autosampler (FTN), a column oven (set at 40° C., unless otherwise indicated), a photo-diode array PDA detector. Chromatography was performed on a AcQuity UPLC BEH C18 (1.7 m 50*2.1 mm) with water containing 0.1% formic acid as solvent A and acetonitrile containing 0.1% formic acid as solvent B at a flow rate of 0.6 m/min. Flow from the column was split to a MS spectrometer. The MS detector was configured with an electrospray ionization source. Nitrogen was used as the nebulizer gas. Data acquisition was performed with a MassLynx data system. Proton Nuclear Magnetic Resonance (¹H-NMR) spectra were recorded on a Bruker Avance 111400 spectrometer. Chemical shifts are expressed in parts per million (ppm) and reported as δ value (chemical shift δ). Coupling constants are reported in units of hertz (J value, Hz; Integration and splitting patterns: where s=singlet, d=double, t=triplet, q=quartet, brs=broad singlet, m=multiple). Preparative HPLC was performed on Agilent Prep 1260 series with UV detector set to 254 nm or 220 nm. Samples were injected onto a Phenomenex Luna 75×30 mm, 5 μm, C18 column at room temperature. The flow rate was 40 mL/min. A linear gradient was used with 10% (or 50%) of MeOH (A) in H₂O (with 0.1% TFA) (B) to 100% of MeOH (A). All compounds showed >90% purity using the LCMS methods described above.

Cell Culture

HEL, RS4;11, MV4;11, Kasumi-1 and other cells were cultured at 37° C. with 5% CO₂ in RPMI 1640 or DMEM Medium supplemented with 10% fetal bovine serum. Cells were authenticated using the short tandem repeat (STR) assays. Mycoplasma test results were negative.

Antibodies and Reagents

Rabbit anti-JAK1 antibody (3344S), anti-JAK2 antibody (3230S), anti-JAK3 antibody (8827S), anti-TYK2 antibody (14193S), anti-FLT3 antibody (3462S), anti-RET antibody (14556S), anti-phospho-STAT3 (Tyr705) antibody (9145S) and anti-phospho-STAT5 (Tyr694) antibody (4322S) were purchased from Cell Signaling Technology. Rabbit anti-GSPT1 antibody (ab126090) was purchased from Abcam. HRP-conjugated anti-α-tubulin antibody was produced in house. Media and other cell culture reagents were purchased from Thermo Fisher. The CellTiter-Glo Luminescent Assay kit was purchased from Promega.

Immunoblotting

Cultured cells were washed with cold PBS once and lysed in cold RIPA buffer supplemented with protease inhibitors and phosphatase inhibitors (Beyotime Biotechnology). The solutions were then incubated at 4° C. for 30 minutes with gentle agitation to fully lyse cells. Cell lysates were centrifuged at 13,000 rpm for 10 minutes at 4° C. and pellets were discarded. Total protein concentrations in the lysates were determined by BCA assays (Beyotime Biotechnology). Cell lysates were mixed with Laemmli loading buffer to 1× and heated at 99° C. for 5 min. Proteins were resolved on SDS-PAGE and visualized by chemiluminescence. Images were taken by a ChemiDoc MP Imaging system (Bio-Rad). Protein bands were quantitated using the accompanied software provided by Bio-Rad.

Cell Viability Assays

Cells were seeded at a density of 5000 cells per well in 96-well assay plates and treated with test compounds following a 8-point or 12-point 3-fold serial dilution. Three days later, cell viability was determined using the CellTiter-Glo assay kit according to the manufacturer's instructions. The dose-response curves were determined and IC₅₀ values were calculated using the GraphPad Prism software following a nonlinear regression (least squares fit) method.

The cell viability inhibition results of selected heterobifunctional compounds are set forth in Table 2 and Table 3 below.

TABLE 2

| ID | IC₅₀ |
|---|---|
| JA-001 | 3169 |
| JA-002 | 5440 |
| JA-003 | 6592 |
| JA-004 | 3348 |
| JA-005 | 510.3 |
| JA-006 | >10000 |
| JA-007 | >10000 |
| JA-008 | 1465 |
| JA-009 | 2580 |
| JA-010 | >10000 |
| JA-011 | 3042 |
| JA-012 | 4701 |
| JA-013 | >10000 |
| JA-014 | 3336 |
| JA-015 | >10000 |
| JA-016 | >10000 |
| JA-017 | >10000 |
| JA-018 | >10000 |
| JA-019 | 357.1 |
| JA-020 | 632.9 |
| JA-021 | 939.7 |
| JA-022 | >10000 |
| JA-023 | >10000 |
| JA-024 | >10000 |
| JA-025 | >10000 |
| JA-026 | >10000 |
| JA-027 | >10000 |
| JA-028 | >10000 |
| JA-029 | >10000 |
| JA-030 | >10000 |
| JA-031 | >10000 |
| JA-032 | >10000 |
| JA-033 | >10000 |
| JA-034 | >10000 |
| JA-035 | >10000 |
| JA-036 | >10000 |
| JA-037 | >10000 |
| JA-038 | >10000 |
| JA-039 | >10000 |
| JA-040 | >10000 |
| JA-041 | >10000 |
| JA-042 | >10000 |
| JA-043 | 7545 |
| JA-044 | >10000 |
| JA-045 | 1262 |
| JA-046 | >10000 |
| JA-047 | 3443 |
| JA-048 | >10000 |
| JA-049 | 1272 |
| JA-050 | >10000 |
| JA-051 | 4637 |
| JA-052 | >10000 |
| JA-053 | >10000 |
| JA-054 | >10000 |
| JA-055 | >10000 |
| JA-056 | >10000 |
| JA-057 | >10000 |
| JA-058 | 2519 |
| JA-059 | >10000 |
| JA-060 | >10000 |
| JA-061 | >10000 |
| JA-062 | >10000 |
| JA-063 | >10000 |
| JA-064 | >10000 |
| JA-065 | 871.6 |
| JA-066 | >10000 |
| JA-067 | 3855 |
| JA-068 | >10000 |
| JA-069 | >10000 |
| JA-070 | 2752 |
| JA-071 | 959.8 |
| JA-072 | 6460 |
| JA-073 | 4589 |
| JA-074 | >10000 |
| JA-075 | 2434 |
| JA-076 | >10000 |
| JA-077 | 406.5 |
| JA-078 | >10000 |
| JA-079 | 478.6 |
| JA-080 | 573.2 |
| JA-081 | >10000 |
| JA-082 | 6433 |
| JA-083 | >10000 |
| JA-084 | >10000 |
| JA-085 | 3954 |
| JA-086 | 563.5 |
| JA-087 | 142.7 |
| JA-088 | 368.9 |
| JA-089 | 974.9 |
| JA-090 | 3483 |
| JA-091 | 2980 |
| JA-092 | 2832 |
| JA-093 | 73.34 |
| JA-094 | 74.43 |
| JA-095 | 121.5 |
| JA-096 | 158.5 |
| JA-097 | >10000 |
| JA-098 | >10000 |
| JA-099 | 3700 |
| JA-100 | >10000 |
| JA-101 | >10000 |
| JA-102 | >10000 |
| JA-103 | 8959 |
| JA-104 | >10000 |
| JA-105 | >10000 |
| JA-106 | >10000 |
| JA-107 | 2270 |
| JA-108 | >10000 |
| JA-109 | >10000 |
| JA-110 | >10000 |
| JA-111 | >10000 |
| JA-112 | >10000 |
| JA-113 | >10000 |
| JA-114 | >10000 |
| JA-115 | 2143 |
| JA-116 | >10000 |
| JA-117 | 1435 |
| JA-118 | >10000 |
| JA-119 | >10000 |
| JA-120 | >10000 |
| JA-121 | >10000 |
| JA-122 | >10000 |
| JA-123 | >10000 |
| JA-124 | >10000 |
| JA-125 | >10000 |
| JA-126 | >10000 |
| JA-127 | >10000 |
| JA-128 | >10000 |
| JA-129 | >10000 |
| JA-130 | >10000 |
| JA-131 | >10000 |
| JA-132 | >10000 |
| JA-133 | >10000 |
| JA-134 | 4309 |
| JA-135 | >10000 |
| JA-136 | >10000 |
| JA-137 | >10000 |
| JA-138 | >10000 |
| JA-139 | >10000 |
| JA-140 | >10000 |
| JA-141 | >10000 |
| JA-142 | >10000 |
| JA-143 | >10000 |
| JA-144 | 2388 |
| JA-145 | >10000 |
| JA-146 | >10000 |
| JA-147 | >10000 |
| JA-148 | >10000 |
| JA-149 | >10000 |
| JA-150 | 2250 |
| JA-151 | >10000 |
| JA-152 | >10000 |
| JA-153 | >10000 |
| JA-154 | 1412 |
| JA-155 | >10000 |
| JA-156 | 3657 |
| JA-157 | >10000 |
| JA-158 | >10000 |
| JA-159 | >10000 |
| JA-160 | >10000 |
| JA-161 | 2371 |
| JA-162 | >10000 |
| JA-163 | >10000 |
| JA-164 | >10000 |
| JA-165 | >10000 |
| JA-166 | 4454 |
| JA-167 | >10000 |
| JA-168 | >10000 |
| JA-169 | 2031 |
| JA-170 | >10000 |
| JA-171 | >10000 |
| JA-172 | >10000 |
| JA-173 | >10000 |
| JA-174 | 2448 |
| JA-175 | 128.3 |
| JA-176 | 2791 |
| JA-177 | 890 |
| JA-178 | 1901 |
| JA-179 | 78.3 |
| JA-180 | 88.6 |
| JA-181 | 1693 |
| JA-182 | 13.8 |
| JA-183 | >10000 |
| JA-184 | 9991 |
| JA-185 | >10000 |
| JA-186 | >10000 |
| JA-187 | 54.4 |
| JA-188 | 69.7 |
| JA-189 | 2.03 |
| JA-190 | 5159 |
| JA-191 | 9496 |
| JA-192 | >10000 |
| JA-193 | >10000 |
| JA-194 | >10000 |
| JA-195 | >10000 |
| JA-196 | 106.8 |
| JA-197 | 5553 |
| JA-198 | 7.3 |
| JA-199 | 32.5 |
| JA-200 | 403.4 |
| JA-201 | 241.2 |
| JA-202 | 108.1 |
| JA-203 | 22.8 |
| JA-204 | >10000 |
| JA-205 | 676 |
| JA-206 | >10000 |
| JA-207 | 1237 |
| JA-208 | 3346 |
| JA-209 | >10000 |
| JA-210 | >10000 |
| JA-211 | >10000 |
| JA-212 | >10000 |
| JA-213 | 8.45 |
| JA-214 | 63.5 |
| JA-215 | >10000 |
| JA-216 | 3409 |
| JA-217 | 5598 |
| JA-218 | >10000 |
| JA-219 | >10000 |
| JA-220 | >10000 |
| JA-221 | >10000 |
| JA-222 | >10000 |
| JA-223 | 9377 |
| JA-224 | 105 |
| JA-225 | 65.8 |
| JA-226 | 365.1 |
| JA-227 | 3112 |
| JA-228 | 2012 |
| JA-229 | >10000 |
| JA-230 | >10000 |
| JA-231 | 80.2 |
| JA-232 | >10000 |
| JA-233 | 287.3 |
| JA-234 | 1587 |
| JA-235 | 848.5 |
| JA-236 | 2639 |
| JA-237 | >10000 |
| JA-238 | 722.6 |
| JA-239 | 743.1 |
| JA-240 | 638.3 |
| JA-241 | 1040 |
| JA-242 | 543.2 |
| JA-243 | 1981 |
| JA-244 | >10000 |
| JA-245 | 150 |
| JA-246 | 228.6 |
| JA-247 | 483.8 |
| JA-248 | 246.6 |
| JA-249 | 103.6 |
| JA-250 | 180.8 |
| JA-251 | 336.7 |
| JA-252 | 24.6 |
| JA-253 | 298 |
| JA-254 | 1149 |
| JA-255 | 259 |
| JA-256 | 1898 |
| JA-257 | 137.2 |
| JA-258 | 647.4 |
| JA-259 | >10000 |
| JA-260 | 164.3 |
| JA-261 | 32.8 |
| JA-262 | >10000 |
| JA-263 | 3.6 |
| JA-264 | 98.6 |
| JA-265 | 1354 |
| JA-266 | 300.9 |
| JA-267 | 426.4 |
| JA-268 | 5.4 |
| JA-269 | 3.8 |
| JA-270 | 561.8 |
| JA-271 | 157 |
| JA-272 | 520 |
| JA-273 | 66 |
| JA-274 | 1667 |
| JA-275 | 175.9 |
| JA-276 | >10000 |
| JA-277 | 446.8 |
| JA-278 | >500 |
| JA-279 | >500 |
| JA-280 | >500 |
| JA-281 | >500 |
| JA-282 | >500 |
| JA-283 | >500 |
| JA-284 | >500 |
| JA-285 | >500 |
| JA-286 | >500 |
| JA-287 | >500 |
| JA-288 | >500 |
| JA-289 | >500 |
| JA-290 | >500 |
| JA-291 | >500 |
| JA-292 | >500 |
| JA-293 | >500 |
| TG101209 | 1839 |
| NVP-BSK805 | >10000 |

The IC₅₀ value (nM) of each compound was determined in RS4; 11 cells as described in Methods and calculated using the GraphPad Prism 5.0 software.

TABLE 3

| | | JA- | JA- | NVP- | |
|---|---|---|---|---|---|
| Cell line | Disease | 189 | 213 | BSK805 | TG101209 |
| HEL | erythro- | 177.4 | 82.12 | 1045 | 1195 |
| | leukemia | | | | |
| RS4; 11 | B-ALL | 2.03 | 8.45 | >10000 | 1839 |
| MOLT-4 | T-ALL | 6.36 | 16.5 | >10000 | 2504 |
| CCRF-CEM | T-ALL | 3.84 | 19.05 | >10000 | 3959 |
| Jurkat | T-ALL | 34.2 | 62.7 | 2515 | 550 |
| MV4; 11 | AML | 7.93 | 19.55 | 390.5 | 193.6 |
| Kasumi-1 | AML | 86.34 | 69.14 | 757.6 | 529.3 |
| NOMO-1 | AML | 53.16 | 81.68 | 6275 | 1303 |
| MOLM-13 | AML | 20.8 | 27.4 | 201.1 | 48.2 |
| HL-60 | APL | 2.54 | 19.2 | >10000 | >10000 |
| MM.1S | Multiple | 2.27 | 23.05 | >10000 | 6110 |
| | Myeloma | | | | |
| AMO-1 | Multiple | 612.2 | 721.8 | >10000 | 4775 |
| | Myeloma | | | | |
| NCI-H929 | Multiple | 105.8 | 123.7 | >10000 | >10000 |
| | Myeloma | | | | |
| MEG-01 | CML | 89.25 | 74.04 | 8871 | 7116 |
| WSU- | DLBCL | 106.1 | 167.9 | >10000 | 1068 |
| DLCL2 | | | | | |
| Pfeiffer | DLBCL | 320.2 | 266.3 | >10000 | >10000 |
| SU-DHL-1 | large cell | 221.9 | 318.6 | >10000 | >10000 |
| | lymphoma | | | | |
| KM12 | colorectal | 2370 | 678.6 | 2863 | |
| | carcinoma | | | | |

The IC₅₀ values (nM) of selected compounds were determined in indicated cells as described in Methods and calculated using the GraphPad Prism 5.0 software.

### REFERENCES

Bondeson, D. P., A. Mares, I. E. Smith, E. Ko, S. Campos, A. H. Miah, K. E. Mulholland, N. Routly, D. L. Buckley, J. L. Gustafson, N. Zinn, P. Grandi, S. Shimamura, G. Bergamini, M. Faelth-Savitski, M. Bantscheff, C. Cox, D. A. Gordon, R. R. Willard, J. J. Flanagan, L. N. Casillas, B. J. Votta, W. den Besten, K. Famm, L. Kruidenier, P. S. Carter, J. D. Harling, I. Churcher and C. M. Crews (2015). “Catalytic in vivo protein knockdown by small-molecule PROTACs.” Nat Chem Biol 11(8): 611-617.
Buckley, D. L. and C. M. Crews (2014). “Small-molecule control of intracellular protein levels through modulation of the ubiquitin proteasome system.” Angew Chem Int Ed Engl 53(9): 2312-2330.
Buckley, D. L., J. L. Gustafson, I. Van Molle, A. G. Roth, H. S. Tae, P. C. Gareiss, W. L. Jorgensen, A. Ciulli and C. M. Crews (2012). “Small-molecule inhibitors of the interaction between the E3 ligase VHL and HIF1alpha.” Angew Chem Int Ed Engl 51(46): 11463-11467.
Buckley, D. L., K. Raina, N. Darricarrere, J. Hines, J. L. Gustafson, I. E. Smith, A. H. Miah, J. D. Harling and C. M. Crews (2015). “HaloPROTACS: Use of Small Molecule PROTACs to Induce Degradation of HaloTag Fusion Proteins.” ACS Chem Biol 10(8): 1831-1837.
Buckley, D. L., I. Van Molle, P. C. Gareiss, H. S. Tae, J. Michel, D. J. Noblin, W. L. Jorgensen, A. Ciulli and C. M. Crews (2012). “Targeting the von Hippel-Lindau E3 ubiquitin ligase using small molecules to disrupt the VHL/HIF-1alpha interaction.” J Am Chem Soc 134(10): 4465-4468.
Chamberlain, P. P., A. Lopez-Girona, K. Miller, G. Carmel, B. Pagarigan, B. Chie-Leon, E. Rychak, L. G. Corral, Y. J. Ren, M. Wang, M. Riley, S. L. Delker, T. Ito, H. Ando, T. Mori, Y. Hirano, H. Handa, T. Hakoshima, T. O. Daniel and B. E. Cathers (2014). “Structure of the human Cereblon-DDB1-lenalidomide complex reveals basis for responsiveness to thalidomide analogs.” Nat Struct Mol Biol 21(9): 803-809.
Chauvin, C., S. Salhi, C. Le Goff, W. Viranaicken, D. Diop and O. Jean-Jean (2005). “Involvement of human release factors eRF3a and eRF3b in translation termination and regulation of the termination complex formation.” Mol Cell Biol 25(14): 5801-5811.
Colligris, B., H. A. Alkozi and J. Pintor (2014). “Recent developments on dry eye disease treatment compounds.” Saudi J Ophthalmol 28(1): 19-30.
Damsky, W. and B. A. King (2017). “JAK inhibitors in dermatology: The promise of a new drug class.” J Am Acad Dermatol 76(4): 736-744.
Davies, T. G., W. E. Wixted, J. E. Coyle, C. Griffiths-Jones, K. Hearn, R. McMenamin, D. Norton, S. J. Rich, C. Richardson, G. Saxty, H. M. Willems, A. J. Woolford, J. E. Cottom, J. P. Kou, J. G. Yonchuk, H. G. Feldser, Y. Sanchez, J. P. Foley, B. J. Bolognese, G. Logan, P. L. Podolin, H. Yan, J. F. Callahan, T. D. Heightman and J. K. Kerns (2016). “Monoacidic Inhibitors of the Kelch-like ECH-Associated Protein 1: Nuclear Factor Erythroid 2-Related Factor 2 (KEAP1:NRF2) Protein-Protein Interaction with High Cell Potency Identified by Fragment-Based Discovery.” J Med Chem 59(8): 3991-4006.
Fischer, E. S., K. Bohm, J. R. Lydeard, H. Yang, M. B. Stadler, S. Cavadini, J. Nagel, F. Serluca, V. Acker, G. M. Lingaraju, R. B. Tichkule, M. Schebesta, W. C. Forrester, M. Schirle, U. Hassiepen, J. Ottl, M. Hild, R. E. Beckwith, J. W. Harper, J. L. Jenkins and N. H. Thoma (2014). “Structure of the DDB1-CRBN E3 ubiquitin ligase in complex with thalidomide.” Nature 512(7512): 49-53.
Fleming, S. B. (2016). “Viral Inhibition of the IFN-Induced JAK/STAT Signalling Pathway: Development of Live Attenuated Vaccines by Mutation of Viral-Encoded IFN-Antagonists.” Vaccines (Basel) 4 (3).
Frolova, L., X. Le Goff, G. Zhouravleva, E. Davydova, M. Philippe and L. Kisselev (1996). “Eukaryotic polypeptide chain release factor eRF3 is an eRF1- and ribosome-dependent guanosine triphosphatase.” RNA 2(4): 334-341.
Galdeano, C., M. S. Gadd, P. Soares, S. Scaffidi, I. Van Molle, I. Birced, S. Hewitt, D. M. Dias and A. Ciulli (2014). “Structure-guided design and optimization of small molecules targeting the protein-protein interaction between the von Hippel-Lindau (VHL) E3 ubiquitin ligase and the hypoxia inducible factor (HIF) alpha subunit with in vitro nanomolar affinities.” J Med Chem 57(20): 8657-8663.
Griesshammer, M. and P. Sadjadian (2017). “The BCR-ABL1-negative myeloproliferative neoplasms: a review of JAK inhibitors in the therapeutic armamentarium.” Expert Opin Pharmacother 18(18): 1929-1938.
Hansen, J. D., M. Correa, M. A. Nagy, M. Alexander, V. Plantevin, V. Grant, B. Whitefield, D. Huang, T. Kercher, R. Harris, R. K. Narla, J. Leisten, Y. Tang, M. Moghaddam, K. Ebinger, J. Piccotti, C. G. Havens, B. Cathers, J. Carmichael, T. Daniel, R. Vessey, L. G. Hamann, K. Leftheris, D. Mendy, F. Baculi, L. A. LeBrun, G. Khambatta and A. Lopez-Girona (2020). “Discovery of CRBN E3 Ligase Modulator CC-92480 for the Treatment of Relapsed and Refractory Multiple Myeloma.” J Med Chem.
Ishoey, M., S. Chom, N. Singh, M. G. Jaeger, M. Brand, J. Paulk, S. Bauer, M. A. Erb, K. Parapatics, A. C. Muller, K. L. Bennett, G. F. Ecker, J. E. Bradner and G. E. Winter (2018). “Translation Termination Factor GSPT1 Is a Phenotypically Relevant Off-Target of Heterobifunctional Phthalimide Degraders.” ACS Chem Biol 13(3): 553-560.
Ito, T., H. Ando, T. Suzuki, T. Ogura, K. Hotta, Y. Imamura, Y. Yamaguchi and H. Handa (2010). “Identification of a primary target of thalidomide teratogenicity.” Science 327(5971): 1345-1350.
Keil, E., D. Finkenstadt, C. Wufka, M. Trilling, P. Liebfried, B. Strobl, M. Muller and K. Pfeffer (2014). “Important scaffold function of the Janus kinase 2 uncovered by a novel mouse model harboring a Jak2 activation-loop mutation.” Blood 123(4): 520-529.
Kilpivaara, O. and R. L. Levine (2008). “JAK2 and MPL mutations in myeloproliferative neoplasms: discovery and science.” Leukemia 22(10): 1813-1817.
Koppikar, P., N. Bhagwat, O. Kilpivaara, T. Manshouri, M. Adli, T. Hricik, F. Liu, L. M. Saunders, A. Mullally, O. Abdel-Wahab, L. Leung, A. Weinstein, S. Marubayashi, A. Goel, M. Gonen, Z. Estrov, B. L. Ebert, G. Chiosis, S. D. Nimer, B. E. Bernstein, S. Verstovsek and R. L. Levine (2012). “Heterodimeric JAK-STAT activation as a mechanism of persistence to JAK2 inhibitor therapy.” Nature 489(7414): 155-159.
LaFave, L. M. and R. L. Levine (2012). “JAK2 the future: therapeutic strategies for JAK-dependent malignancies.” Trends Pharmacol Sci 33(11): 574-582.
Lai, A. C., M. Toure, D. Hellerschmied, J. Salami, S. Jaime-Figueroa, E. Ko, J. Hines and C. M. Crews (2016). “Modular PROTAC Design for the Degradation of Oncogenic BCR-ABL.” Angew Chem Int Ed Engl 55(2): 807-810.
Langabeer, S. E. (2014). “JAK2 mutations to the fore in hereditary thrombocythemia.” JAKSTAT 3 (3): e957618.
Levine, R. L., M. Wadleigh, J. Cools, B. L. Ebert, G. Wernig, B. J. Huntly, T. J. Boggon, I. Wlodarska, J. J. Clark, S. Moore, J. Adelsperger, S. Koo, J. C. Lee, S. Gabriel, T. Mercher, A. D'Andrea, S. Frohling, K. Dohner, P. Marynen, P. Vandenberghe, R. A. Mesa, A. Tefferi, J. D. Griffin, M. J. Eck, W. R. Sellers, M. Meyerson, T. R. Golub, S. J. Lee and D. G. Gilliland (2005). “Activating mutation in the tyrosine kinase JAK2 in polycythemia vera, essential thrombocythemia, and myeloid metaplasia with myelofibrosis.” Cancer Cell 7(4): 387-397.
Li, J. (2013). “JAK-STAT and bone metabolism.” JAKSTAT 2 (3): e23930.
Lu, J., Y. Qian, M. Altieri, H. Dong, J. Wang, K. Raina, J. Hines, J. D. Winkler, A. P. Crew, K. Coleman and C. M. Crews (2015). “Hijacking the E3 Ubiquitin Ligase Cereblon to Efficiently Target BRD4.” Chem Biol 22(6): 755-763.
Malta-Vacas, J., C. Aires, P. Costa, A. R. Conde, S. Ramos, A. P. Martins, C. Monteiro and M. Brito (2005). “Differential expression of the eukaryotic release factor 3 (eRF3/GSPT1) according to gastric cancer histological types.” J Clin Pathol 58(6): 621-625.
Matyskiela, M. E., G. Lu, T. Ito, B. Pagarigan, C. C. Lu, K. Miller, W. Fang, N. Y. Wang, D. Nguyen, J. Houston, G. Carmel, T. Tran, M. Riley, L. Nosaka, G. C. Lander, S. Gaidarova, S. Xu, A. L. Ruchelman, H. Handa, J. Carmichael, T. O. Daniel, B. E. Cathers, A. Lopez-Girona and P. P. Chamberlain (2016). “A novel cereblon modulator recruits GSPT1 to the CRL4 (CRBN) ubiquitin ligase.” Nature 535(7611): 252-257.
Maxson, J. E., J. Gotlib, D. A. Pollyea, A. G. Fleischman, A. Agarwal, C. A. Eide, D. Bottomly, B. Wilmot, S. K. McWeeney, C. E. Tognon, J. B. Pond, R. H. Collins, B. Goueli, S. T. Oh, M. W. Deininger, B. H. Chang, M. M. Loriaux, B. J. Druker and J. W. Tyner (2013). “Oncogenic CSF3R mutations in chronic neutrophilic leukemia and atypical CML.” N Engl J Med 368(19): 1781-1790.
Moore, C. A., C. J. Iasella, R. Venkataramanan, F. G. Lakkis, R. B. Smith, J. F. McDyer, A. Zeevi and C. R. Ensor (2017). “Janus kinase inhibition for immunosuppression in solid organ transplantation: Is there a role in complex immunologic challenges?” Hum Immunol 78(2): 64-71.
Neubauer, H., A. Cumano, M. Muller, H. Wu, U. Huffstadt and K. Pfeffer (1998). “Jak2 deficiency defines an essential developmental checkpoint in definitive hematopoiesis.” Cell 93(3): 397-409.
O'Shea, J. J., S. M. Holland and L. M. Staudt (2013). “JAKs and STATs in immunity, immunodeficiency, and cancer.” N Engl J Med 368(2): 161-170.
O'Shea, J. J., A. Kontzias, K. Yamaoka, Y. Tanaka and A. Laurence (2013). “Janus kinase inhibitors in autoimmune diseases.” Ann Rheum Dis 72 Suppl 2: ii111-115.
O'Shea, J. J., D. M. Schwartz, A. V. Villarino, M. Gadina, I. B. McInnes and A. Laurence (2015). “The JAK-STAT pathway: impact on human disease and therapeutic intervention.” Annu Rev Med 66: 311-328.
Ohoka, N., K. Okuhira, M. Ito, K. Nagai, N. Shibata, T. Hattori, O. Ujikawa, K. Shimokawa, O. Sano, R. Koyama, H. Fujita, M. Teratani, H. Matsumoto, Y. Imaeda, H. Nara, N. Cho and M. Naito (2017). “In Vivo Knockdown of Pathogenic Proteins via Specific and Nongenetic Inhibitor of Apoptosis Protein (IAP)-dependent Protein Erasers (SNIPERs).” J Biol Chem 292(11): 4556-4570.
Okuhira, K., N. Ohoka, K. Sai, T. Nishimaki-Mogami, Y. Itoh, M. Ishikawa, Y. Hashimoto and M. Naito (2011). “Specific degradation of CRABP-II via cIAP1-mediated ubiquitylation induced by hybrid molecules that crosslink cIAP1 and the target protein.” FEBS Lett 585(8): 1147-1152.
Rumi, E., D. Pietra, C. Pascutto, P. Guglielmelli, A. Martinez-Trillos, I. Casetti, D. Colomer, L. Pieri, M. Pratcorona, G. Rotunno, E. Sant'Antonio, M. Bellini, C. Cavalloni, C. Mannarelli, C. Milanesi, E. Boveri, V. Ferretti, C. Astori, V. Rosti, F. Cervantes, G. Barosi, A. M. Vannucchi, M. Cazzola and I. Associazione Italiana per la Ricerca sul Cancro Gruppo Italiano Malattie Mieloproliferative (2014). “Clinical effect of driver mutations of JAK2, CALR, or MPL in primary myelofibrosis.” Blood 124(7): 1062-1069.
Schwartz, D. M., Y. Kanno, A. Villarino, M. Ward, M. Gadina and J. J. O'Shea (2017). “JAK inhibition as a therapeutic strategy for immune and inflammatory diseases.” Nat Rev Drug Discov 16(12): 843-862.
Shibata, N., N. Miyamoto, K. Nagai, K. Shimokawa, T. Sameshima, N. Ohoka, T. Hattori, Y. Imaeda, H. Nara, N. Cho and M. Naito (2017). “Development of protein degradation inducers of oncogenic BCR-ABL protein by conjugation of ABL kinase inhibitors and IAP ligands.” Cancer Sci 108(8): 1657-1666.
Sun, D., Z. Li, Y. Rew, M. Gribble, M. D. Bartberger, H. P. Beck, J. Canon, A. Chen, X. Chen, D. Chow, J. Deignan, J. Duquette, J. Eksterowicz, B. Fisher, B. M. Fox, J. Fu, A. Z. Gonzalez, F. Gonzalez-Lopez De Turiso, J. B. Houze, X. Huang, M. Jiang, L. Jin, F. Kayser, J. J. Liu, M. C. Lo, A. M. Long, B. Lucas, L. R. McGee, J. McIntosh, J. Mihalic, J. D. Oliner, T. Osgood, M. L. Peterson, P. Roveto, A. Y. Saiki, P. Shaffer, M. Toteva, Y. Wang, Y. C. Wang, S. Wortman, P. Yakowec, X. Yan, Q. Ye, D. Yu, M. Yu, X. Zhao, J. Zhou, J. Zhu, S. H. Olson and J. C. Medina (2014). “Discovery of AMG 232, a potent, selective, and orally bioavailable MDM2-p53 inhibitor in clinical development.” J Med Chem 57(4): 1454-1472.
Sun, W., L. Zhang, R. Yan, Y. Yang and X. Meng (2019). “LncRNA DLX6-AS1 promotes the proliferation, invasion, and migration of non-small cell lung cancer cells by targeting the miR-27b-3p/GSPT1 axis.” Onco Targets Ther 12: 3945-3954.
Tefferi, A. (2012). “JAK inhibitors for myeloproliferative neoplasms: clarifying facts from myths.” Blood 119(12): 2721-2730.
Tian, Q. G., R. C. Tian, Y. Liu, A. Y. Niu, J. Zhang and W. F. Gao (2018). “The role of miR-144/GSPT1 axis in gastric cancer.” Eur Rev Med Pharmacol Sci 22(13): 4138-4145.
Villarino, A. V., Y. Kanno, J. R. Ferdinand and J. J. O'Shea (2015). “Mechanisms of Jak/STAT signaling in immunity and disease.” J Immunol 194(1): 21-27.
Winter, G. E., D. L. Buckley, J. Paulk, J. M. Roberts, A. Souza, S. Dhe-Paganon and J. E. Bradner (2015). “Phthalimide conjugation as a strategy for in vivo target protein degradation.” Science 348(6241): 1376-1381.
Xie, T., S. M. Lim, K. D. Westover, M. E. Dodge, D. Ercan, S. B. Ficarro, D. Udayakumar, D. Gurbani, H. S. Tae, S. M. Riddle, T. Sim, J. A. Marto, P. A. Janne, C. M. Crews and N. S. Gray (2014). “Pharmacological targeting of the pseudokinase Her3.” Nat Chem Biol 10(12): 1006-1012.
Zengerle, M., K. H. Chan and A. Ciulli (2015). “Selective Small Molecule Induced Degradation of the BET Bromodomain Protein BRD4.” ACS Chem Biol 10(8): 1770-1777.
Zhang, C., Y. Zou and D. Q. Dai (2019). “Downregulation of microRNA-27b-3p via aberrant DNA methylation contributes to malignant behavior of gastric cancer cells by targeting GSPT1.” Biomed Pharmacother 119: 109417.
Zhouravleva, G., L. Frolova, X. Le Goff, R. Le Guellec, S. Inge-Vechtomov, L. Kisselev and M. Philippe (1995). “Termination of translation in eukaryotes is governed by two interacting polypeptide chain release factors, eRF1 and eRF3.” EMBO J 14(16): 4065-4072.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A heterobifunctional compound comprising a Janus kinase ligand (JAK ligand) conjugated to a degradation tag, or a pharmaceutically acceptable salt or analog thereof, wherein the JAK ligand is conjugated to the degradation tag via a linker moiety; and
(I) wherein the JAK ligand comprises a moiety of FORMULA 1: 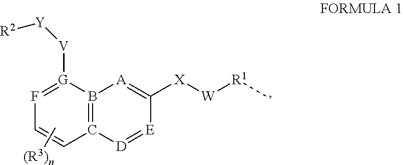
wherein
the linker moiety of the heterobifunctional compound is attached to R¹;
A and D are independently selected from CR⁴ and N, wherein
R⁴ is selected from hydrogen, halogen, optionally substituted C₁-C₈ alkyl, and optionally substituted 3-10 membered carbocyclyl;
B, C and G are independently selected from C and N; with the proviso that at most only one of B, C and G is N;
E and F are independently selected from null, CR⁵ and N, wherein
R⁵ is selected from hydrogen, halogen, optionally substituted C₁-C₈ alkyl, and optionally substituted 3-10 membered carbocyclyl;
X and Y are independently selected from null, or a bivalent moiety selected from null, CR⁶R⁷, CO, CO₂, CONR⁶, NR⁶, NR⁶CO, NR⁶CO₂, NR⁶C(O)NR⁷, NR⁶SO, NR⁶SO₂, NR⁶SO₂NR⁷, O, OC(O), OCO₂, OCONR⁶, S, SO, SO₂, and SO₂NR⁶, wherein
R⁶ and R⁷ are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkylamino, optionally substituted 3-10 membered carbocyclyl, optionally substituted 4-10 membered heterocyclyl, optionally substituted aryl, and optionally substituted heteroaryl, or
R⁶ and R⁷ together with the atom or atoms to which they are connected form a 3-20 membered carbocyclyl ring or 4-20 membered heterocyclyl ring;
V and W are independently selected from null, carbocyclyl, heterocyclyl, aryl, and heteroaryl, which are optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein
R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or
R⁸ and R⁹, R⁸ and R¹⁰ together with the atom to which they are connected form a 4-20 membered heterocyclyl ring;
R¹ is connected to the “linker” moiety of the heterobifunctional compound, and is selected from null, R′—R″, R′OR″, R′SR″, R′N(R¹¹)R″, R′OC(O)R″, R′OC(O)OR″, R′OCON(R¹¹)R″, R′C(O)R″, R′C(O)OR″, R′CON(R¹¹)R″, R′S(O)R″, R′S(O)₂R″, R′SO₂N(R¹¹)R″, R′NR¹²C(O)OR″, R′NR¹²C(O)R″, R′NR¹²C(O)N(R¹¹)R″, R′NR¹²S(O)R″, R′NR¹²S(O)₂R″, and R′NR¹²S(O)₂N(R¹¹)R″, wherein
R′ and R″ are independently selected from null, optionally substituted C₁-C₈ alkyl, optionally substituted C₂-C₈ alkenyl, optionally substituted C₂-C₈ alkynyl, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted C₃-C₁₃ fused carbocyclyl, optionally substituted C₄-C₁₃ fused heterocyclyl, optionally substituted C₃-C₁₃ bridged carbocyclyl, optionally substituted C₄-C₁₃ bridged heterocyclyl, optionally substituted C₃-C₁₃ spiro carbocyclyl, optionally substituted C₄-C₁₃ spiro heterocyclyl, optionally substituted aryl, and optionally substituted heteroaryl,
R¹¹ and R¹² are independently selected from optionally substituted C₁-C₈ alkyl, optionally substituted C₂-C₈ alkenyl, optionally substituted C₂-C₈ alkynyl, optionally substituted 3-10 membered carbocyclyl, optionally substituted 4-10 membered heterocyclyl, optionally substituted aryl, and optionally substituted heteroaryl; or
R′ and R″, R¹¹ and R¹², R′ and R¹¹, R′ and R¹², R″ and R¹¹, R″ and R¹², together with the atom to which they are connected, form a 3-20 membered carbocyclyl or 4-20 membered heterocyclyl ring;
R² is selected from hydrogen, halogen, hydroxyl, amino, cyano, nitro, optionally substituted C₁-C₈ alkyl, optionally substituted C₂-C₈ alkenyl, optionally substituted C₂-C₈ alkenyl, optionally substituted 3-10 membered carbocyclyl, optionally substituted 4-10 membered heterocyclyl, optionally substituted aryl, and optionally substituted heteroaryl;
R³, at each occurrence, is selected from hydrogen, halogen, optionally substituted C₁-C₈ alkyl, and optionally substituted 3-10 membered carbocyclyl; and
n is selected from 1 or 2.

2-4. (canceled)

5. The heterobifunctional compound of claim 1, wherein the degradation tag binds to a ubiquitin ligase, or is a hydrophobic group or a tag that leads to misfolding of the JAK protein.

6. The heterobifunctional compound of claim 5, wherein the ubiquitin ligase is an E3 ligase.

7. The heterobifunctional compound of claim 5, wherein the E3 ligase is selected from the group consisting of a cereblon E3 ligase, an IAP ligase, a VHL E3 ligase, a MDM2 ligase, a TRIM24 ligase, a TRIM21 ligase, a KEAP1 ligase, DCAF16 ligase, RNF4 ligase, RNF114 ligase, and AhR ligase.

8. The heterobifunctional compound of claim 5, wherein the degradation tag is selected from the group consisting of pomalidomide, thalidomide, lenalidomide, VHL-1, adamantane, 1-((4,4,5,5,5-pentafluoropentyl)sulfinyl)nonane, nutlin-3a, RG7112, RG7338, AMG232, AA-115, bestatin, MV-1, LCL161, CPD36, GDC-0152, CRBN-1, CRBN-2, CRBN-3, CRBN-4, CRBN-5, CRBN-6, CRBN-7, CRBN-8, CRBN-9, CRBN-10, CRBN-11, and analogs thereof.

9-10. (canceled)

11. The heterobifunctional compound of claim 1, wherein V is Ar²; and the JAK ligand comprises a moiety of FORMULA 1A: 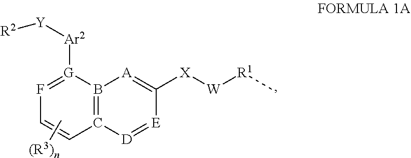
wherein
the linker moiety of the heterobifunctional compound is attached to R¹;
A, B, C, D, E, F, G, X, Y, W, R¹, R², R³, and n are the same as defined in FORMULA 1; and
Ar² is selected from null, aryl, and heteroaryl, each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein
R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or
R⁸ and R⁹, R⁸ and R¹⁰ together with the atom to which they are connected form a 4-20 membered heterocyclyl ring.

12. The heterobifunctional compound of claim 11, wherein
W is Ar¹; and the JAK ligand comprises a moiety of FORMULA 1B: 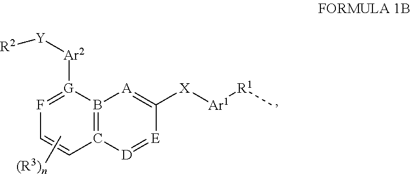
wherein
the linker moiety of the heterobifunctional compound is attached to R¹;
A, B, C, D, E, F, G, X, Y, R¹, R², R³, and n are the same as defined in FORMULA 1; and
Ar¹ and Ar² are independently selected from null, aryl, and heteroaryl, each of which is optionally substituted with one or more substituents independently selected from hydrogen, halogen, oxo, CN, NO₂, OR⁸, SR⁸, NR⁸R⁹, OCOR⁸, OCO₂R⁸, OCONR⁸R⁹, COR⁸, CO₂R⁸, CONR⁸R⁹, SOR⁸, SO₂R⁸, SO₂NR⁸R⁹, NR¹⁰CO₂R⁸, NR¹⁰COR⁸, NR¹⁰C(O)NR⁸R⁹, NR¹⁰SOR⁸, NR¹⁰SO₂R⁸, NR¹⁰SO₂NR⁸R⁹, optionally substituted C₁-C₈ alkyl, optionally substituted C₂-C₈ alkenyl, optionally substituted C₂-C₈ alkynyl, optionally substituted C₁-C₈alkoxyC₁-C₈alkyl, optionally substituted C₁-C₈alkylaminoC₁-C₈alkyl, optionally substituted 4-10 membered heterocyclylC₁-C₈alkyl, optionally substituted 3-10 membered carbocyclylC₁-C₈alkyl, optionally substituted 3-10 membered carbocyclyl, optionally substituted 4-10 membered heterocyclyl, optionally substituted aryl, and optionally substituted heteroaryl, wherein
R⁸, R⁹, and R¹⁰ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈alkoxyC₁-C₈alkyl, optionally substituted C₁-C₈alkylaminoC₁-C₈alkyl, optionally substituted 3-10 membered carbocyclylC₁-C₈alkyl, optionally substituted 4-10 membered heterocyclylC₁-C₈alkyl, optionally substituted 3-10 membered carbocyclyl, optionally substituted 4-10 membered heterocyclyl, optionally substituted aryl, and optionally substituted heteroaryl, or
R⁸ and R⁹, R⁸ and R¹⁰ together with the atom to which they are connected form a 4-20 membered heterocyclyl ring.

13. The heterobifunctional compound of claim 1, wherein
A is N; and the JAK ligand comprises a moiety of FORMULA 1C: 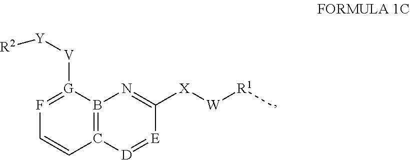
wherein
the linker moiety of the heterobifunctional compound is attached to R¹; and
B, C, D, E, F, G, X, Y, V, W, R¹, R², R³, and n are the same as defined in FORMULA 1.

14. The heterobifunctional compound of claim 13, wherein
V is Ar²; and the JAK ligand comprises a moiety of FORMULA 1D: 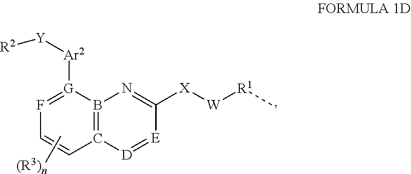
wherein
the linker moiety of the heterobifunctional compound is attached to R¹;
B, C, D, E, F, G, W, X, Y, R¹, R², R³, and n are the same as defined in FORMULA 1; and
Ar² is the same as defined in FORMULA 1A.

15. The heterobifunctional compound of claim 14, wherein
W is Ar¹; and the JAK ligand comprises a moiety of FORMULA 1E: 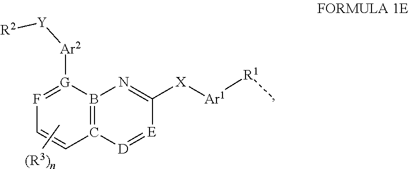
wherein
the linker moiety of the heterobifunctional compound is attached to R¹;
B, C, D, E, F, G, X, Y, R¹, R², R³, and n are the same as defined in FORMULA 1, and
Ar¹ and Ar² are the same as defined in FORMULA 1B.

16. The heterobifunctional compound of claim 1, wherein
the JAK ligand comprises a moiety of FORMULAE 1F, 1G, 1H, or 1I: 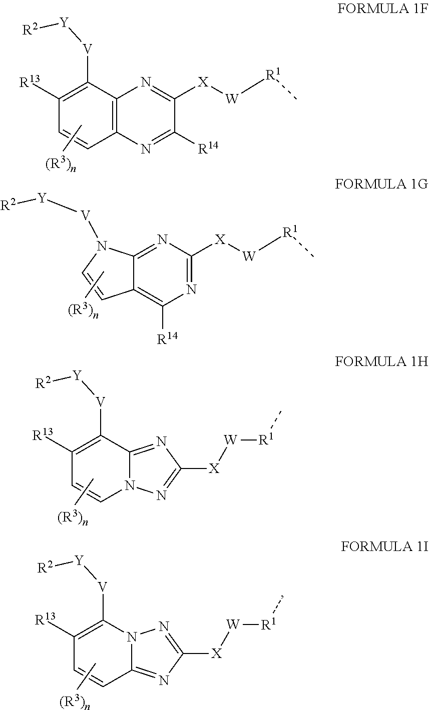
wherein
the linker moiety of the heterobifunctional compound is attached to R¹;
V, W, X, Y, R¹, R², R³, and n are the same as defined in FORMULA 1; and
R¹³ and R¹⁴ are selected from hydrogen, halogen, optionally substituted C₁-C₈ alkyl, and optionally substituted 3-10 membered carbocyclyl.

17. The heterobifunctional compound of claim 16, wherein
V is Ar²; and the JAK ligand comprises a moiety of FORMULAE 1J, 1K, 1L, or 1M: 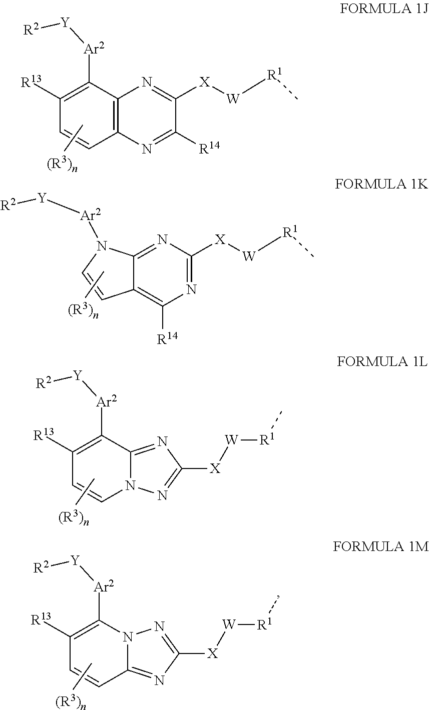
wherein
the linker moiety of the heterobifunctional compound is attached to R¹;
W, X, Y, R¹, R², R³, and n are the same as defined in FORMULA 1;
Ar² is the same as defined in FORMULA 1A; and
R¹³ and R¹⁴ are the same as defined in FORMULAE 1F, 1G, 1H, or 1I.

18. The heterobifunctional compound of claim 17, wherein
W is Ar¹; and the JAK ligand comprises a moiety of FORMULAE 1N, 1O, 1P, or 1Q: 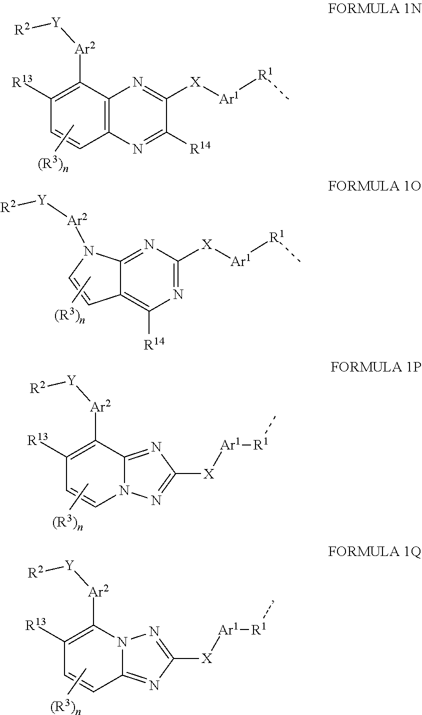
wherein
the linker moiety of the heterobifunctional compound is attached to R¹;
X, Y, R¹, R², R³, and n are the same as defined in FORMULA 1;
Ar¹ and Ar² are the same as defined in FORMULA 1B; and
R¹³ and R¹⁴ are the same as defined in FORMULAE 1F, 1G, 1H, or 1I.

19. The heterobifunctional compound of claim 1, wherein
X is selected from null, O, and NR⁶, wherein
R⁶ is selected from hydrogen, optionally substituted C₁-C₈ alkyl, and optionally substituted 3-10 membered carbocyclyl;
and/or
Y is selected from null, CR^{6′}R⁷, CO, CO₂, O, SO, SO₂, and NR^{6′}, wherein
R^{6′} and R⁷ are independently selected from hydrogen, optionally substituted C₁-C₈ alkyl, optionally substituted 3-10 membered carbocyclyl, and optionally substituted 3-10 membered heterocyclyl;
and/or
R¹ is selected from null, O, NH, CO, CONH, optionally substituted C₁-C₈ alkylene, optionally substituted C₂-C₈ alkenylene, optionally substituted C₂-C₈ alkynylene, optionally substituted 3-10 membered carbocyclyl, optionally substituted 4-10 membered heterocyclyl, optionally substituted aryl, and optionally substituted heteroaryl;
and/or
R² is selected from hydrogen, halogen, optionally substituted C₁-C₈ alkyl, optionally substituted C₂-C₈ alkenyl, optionally substituted C₂-C₈ alkenyl, optionally substituted 3-10 membered carbocyclyl, and optionally substituted 4-10 membered heterocyclyl.

20-22. (canceled)

23. The heterobifunctional compound of claim 15, wherein
Ar¹ and Ar² are independently selected from null, aryl and heteroaryl, each of which is optionally substituted with one or more substituents independently selected from hydrogen, halogen, oxo, CN, NO₂, OR⁸, SR⁸, NR⁸R⁹, COR⁸, CO₂R⁸, CONR⁸R⁹, SOR⁸, SO₂R⁸, SO₂NR⁸R⁹, NR¹⁰COR⁸, NR¹⁰SOR⁸, NR¹⁰SO₂R⁸, optionally substituted C₁-C₈ alkyl, optionally substituted 3-10 membered carbocyclyl, and optionally substituted 4-10 membered heterocyclyl, wherein
R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or
R⁸ and R⁹, R⁸ and R¹⁰ together with the atom to which they are connected form a 4-10 membered heterocyclyl ring.

24. The heterobifunctional compound of claim 23, wherein
Ar¹ and Ar² are independently selected from null, aryl and heteroaryl, each of which is optionally substituted with one or more substituents independently selected from hydrogen, halogen, oxo, CN, NO₂, OR⁸, NR⁸R⁹, NR¹⁰COR⁸, optionally substituted C₁-C₈ alkyl, optionally substituted 3-10 membered carbocyclyl, and optionally substituted 4-10 membered heterocyclyl, wherein
R⁸, R⁹, and R¹⁰ are independently selected from hydrogen, optionally substituted C₁-C₈ alkyl, optionally substituted 3-10 membered carbocyclyl, and optionally substituted 4-10 membered heterocyclyl, or
R⁸ and R⁹, R⁸ and R¹⁰ together with the atom to which they are connected form a 4-10 membered heterocyclyl ring.

25. The heterobifunctional compound of claim 24, wherein
Ar¹ and Ar² are independently selected from null, aryl and heteroaryl, each of which is optionally substituted with one or more substituents independently selected from hydrogen, CH₃, CF₃, iPr, cPr, OCH₃, OCF₃, OiPr, OcPr, F, Cl, and Br.

26. The heterobifunctional compound of claim 25, wherein
Ar¹ and Ar² are independently selected from null, monocyclic aryl, monocyclic heteroaryl, bicyclic aryl, bicyclic heteroaryl, tricyclic aryl, and tricyclic heteroaryl, each of which is optionally substituted with one or more substituents independently selected from H and F.

27. (canceled)

28. The heterobifunctional compound of claim 1, wherein
R¹ is selected from null, optionally substituted 3-10 membered carbocyclyl, and optionally substituted 4-10 membered heterocyclyl.

29. The heterobifunctional compound of claim 1, wherein
R¹ is selected from null and optionally substituted 4-10 membered heterocyclyl, which contains at least one of O or N; and/or
R² is selected from hydrogen, halogen, optionally substituted C₁-C₈ alkyl, optionally substituted 3-10 membered carbocyclyl, and optionally substituted 4-10 membered heterocyclyl.

30-33. (canceled)

34. The heterobifunctional compound of claim 1, wherein
X is selected from null and NH; and/or
Y is selected from null, CH₂, CO, and SO₂; and/or
R¹ is selected from null, optionally substituted piperidinyl, optionally substituted piperazinyl, optionally substituted morpholinyl, optionally substituted pyrrolidinyl, optionally substituted tetrahydrofuranyl, optionally substituted azetidinyl, and optionally substituted oxetanyl; and/or
R² is selected from CH₃, CF₃, iPr, cPr, F, Cl, Br, optionally substituted piperidinyl, optionally substituted optionally substituted piperazinyl, optionally substituted morpholinyl, optionally substituted pyrrolidinyl, optionally substituted tetrahydrofuranyl, optionally substituted azetidinyl, and optionally substituted oxetanyl; and/or
R³, at each occurrence, R¹³ and R¹⁴ are independently selected from hydrogen, CH₃, CF₃, iPr, cPr, tBu, CNCH₂, F, Cl, Br, OH, NH₂, CN, CH₃, and CONH₂.

35-53. (canceled)

54. The heterobifunctional compound of claim 1, wherein the JAK ligand is derived from any of the following:

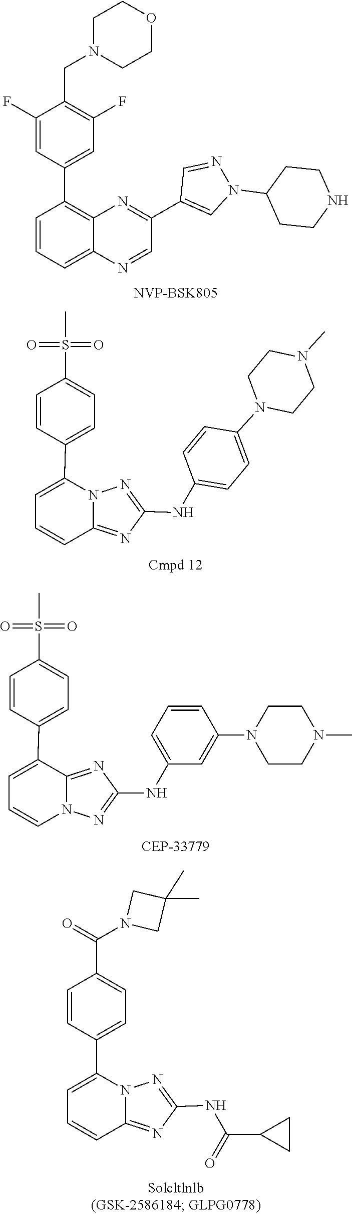

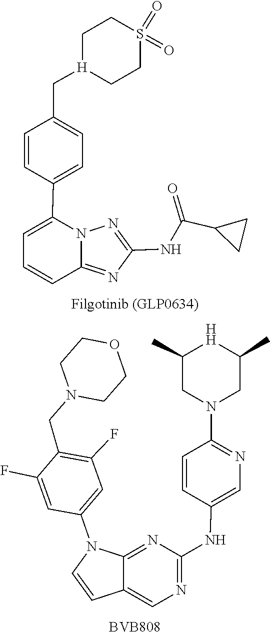

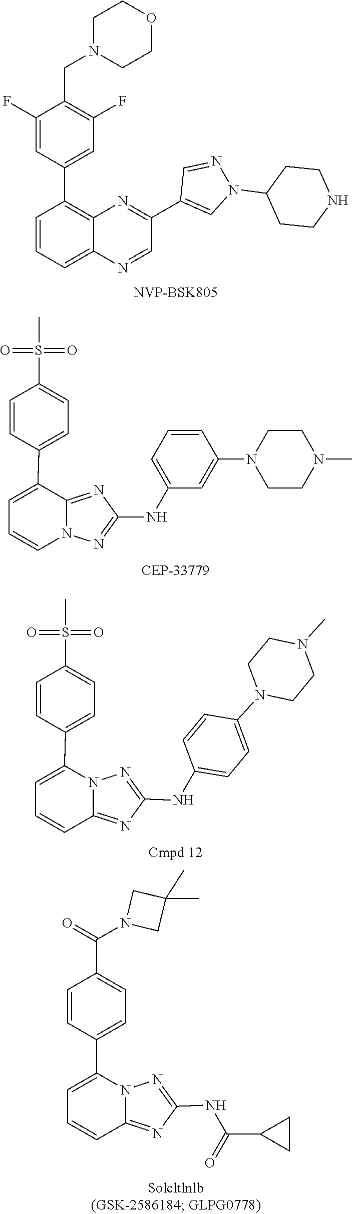

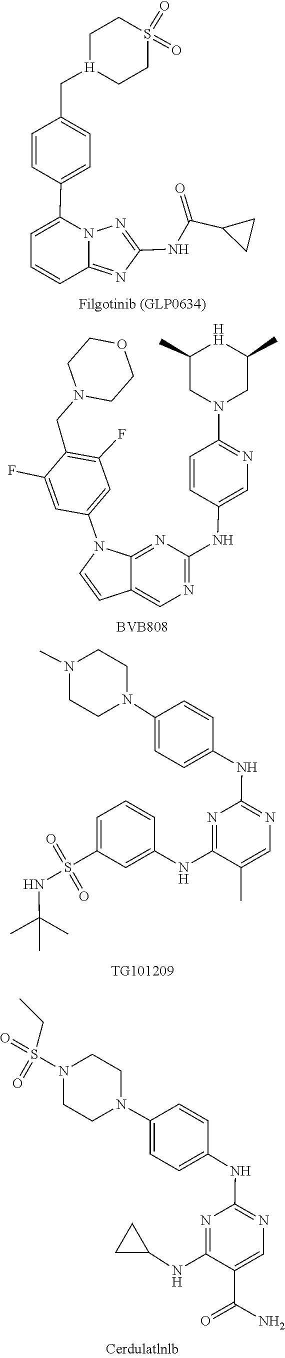

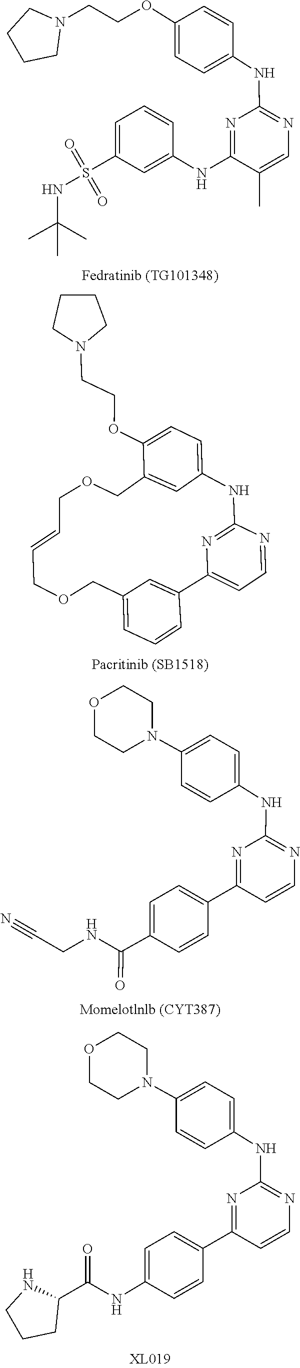

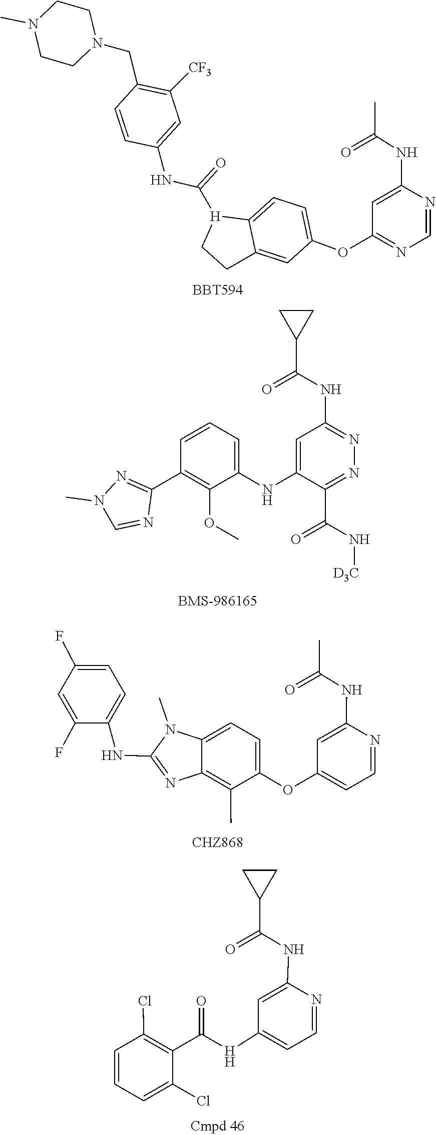

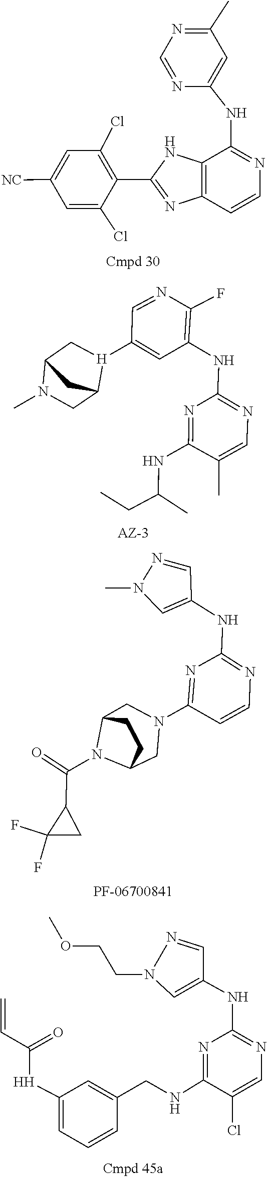

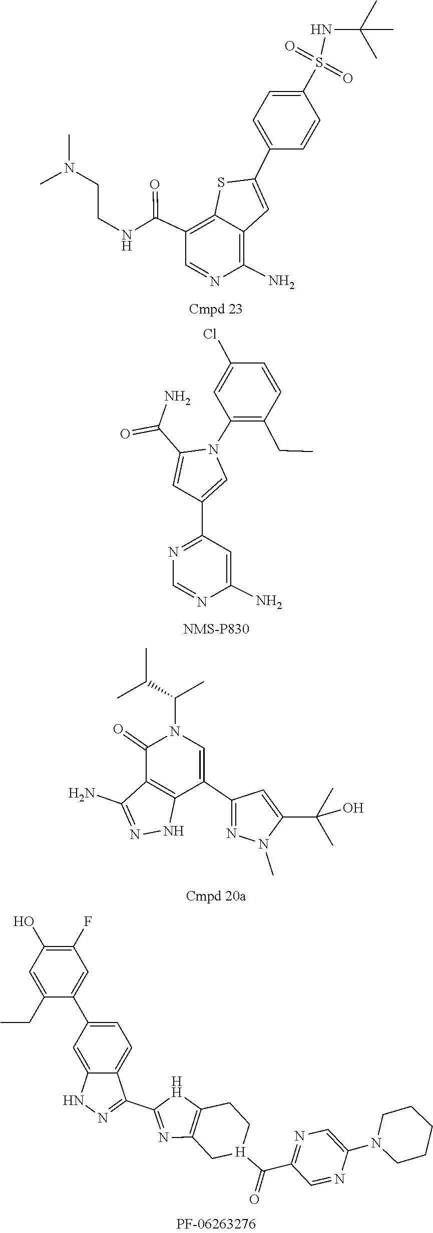

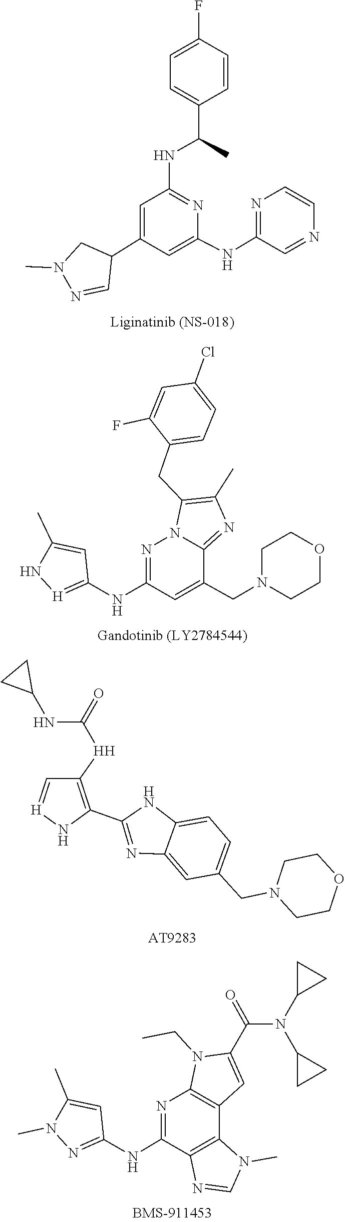

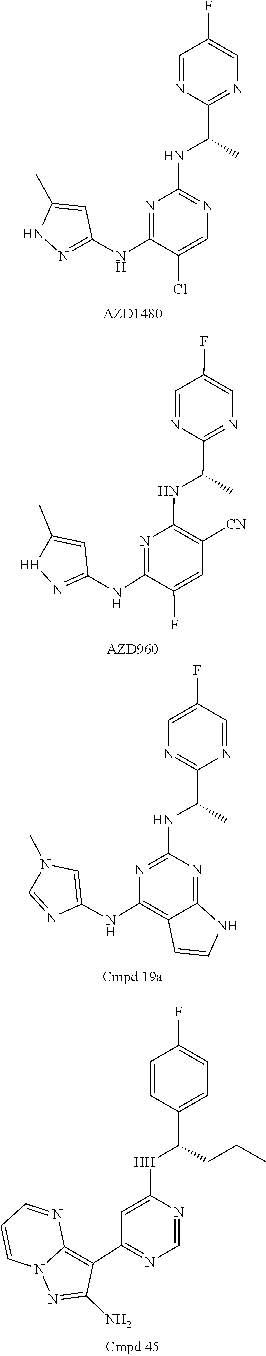

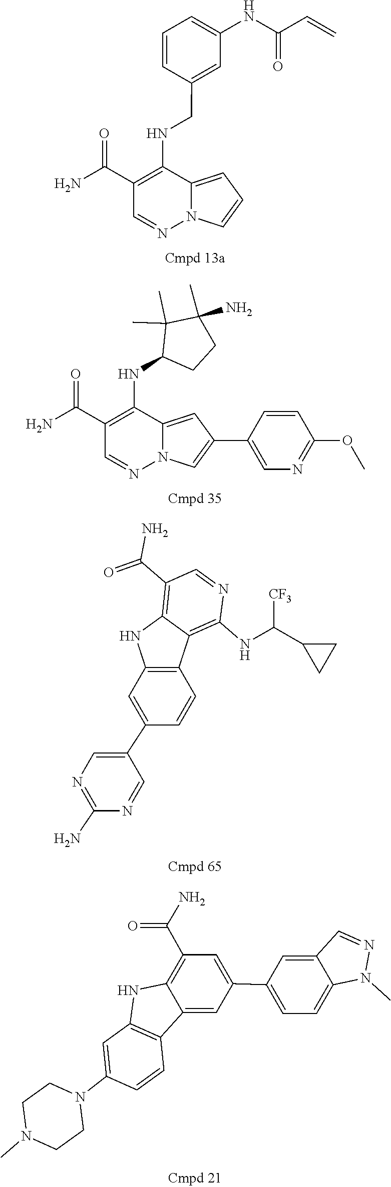

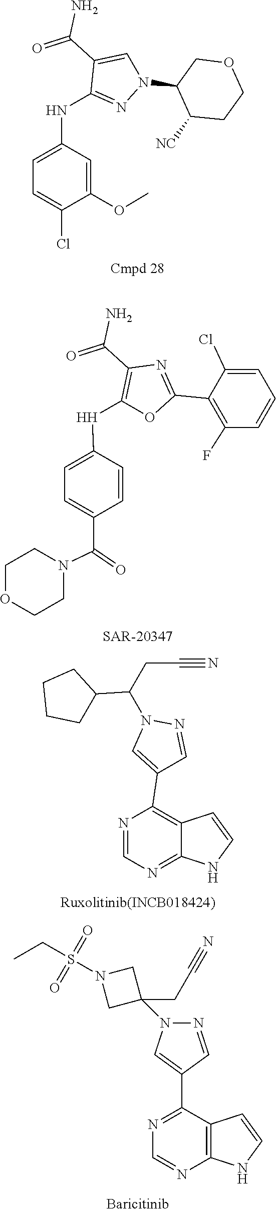

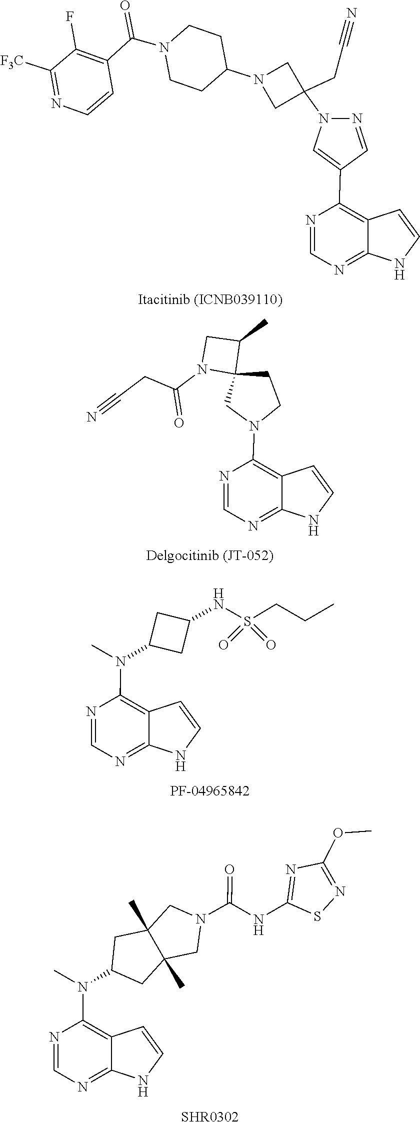

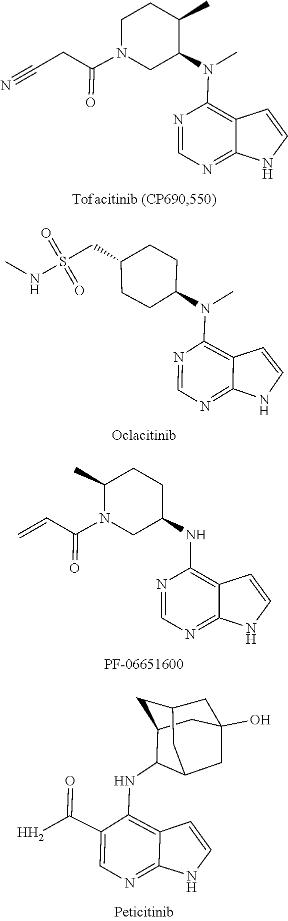 [SMILES: C[C@@H]1CCN(C(=O)CC#N)C[C@@H]1N(C)c1ncnc2[nH]ccc12 ; CNS(=O)(=O)C[C@H]1CC[C@H](N(C)c2ncnc3[nH]ccc23)CC1 ; C=CC(=O)N1C[C@H](Nc2ncnc3[nH]ccc23)CC[C@@H]1C ; O=Cc1cnc2[nH]ccc2c1NC1C2CC3C[C@H]1CC(O)(C3)C2]

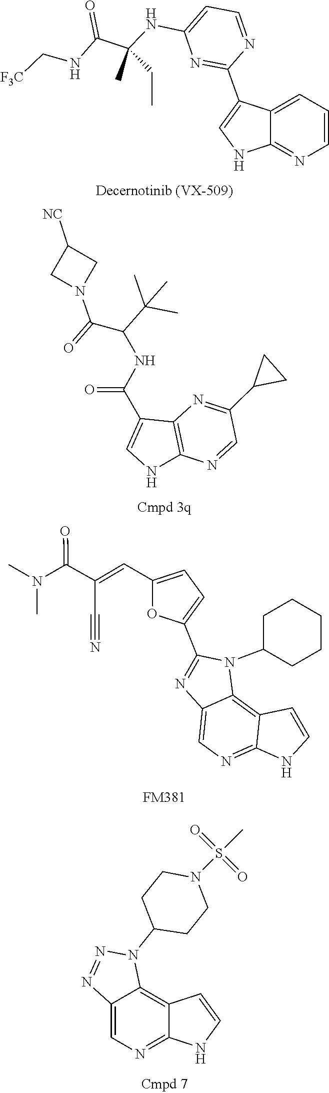

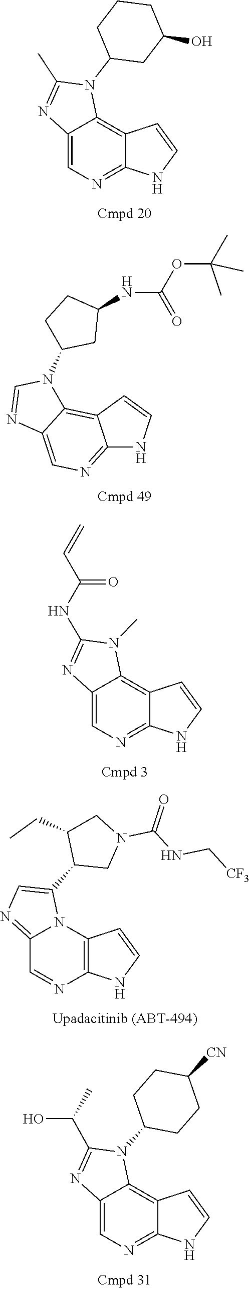 [SMILES: Cc1nc2cnc3[nH]ccc3c2n1C1CCC[C@@H](O)C1 ; CC(C)(C)OC(=O)N[C@@H]1CC[C@@H](n2cnc3cnc4[nH]ccc4c32)C1 ; C=CC(=O)Nc1nc2cnc3[nH]ccc3c2n1C ; CC[C@@H]1CN(C(=O)NCC(F)(F)F)C[C@@H]1c1cnc2cnc3[nH]ccc3n12 ; C[C@@H](O)c1nc2cnc3[nH]ccc3c2n1[C@H]1CC[C@H](C#N)CC1]

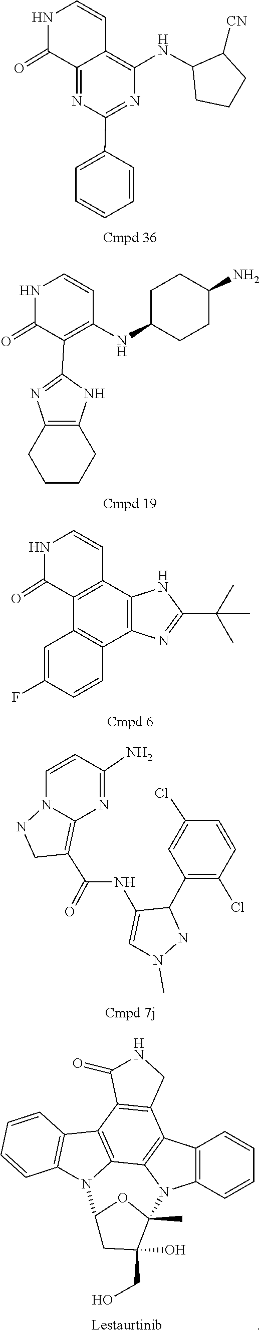

55. (canceled)

56. The heterobifunctional compound of claim 19, wherein the JAK ligand is selected from the group consisting of:

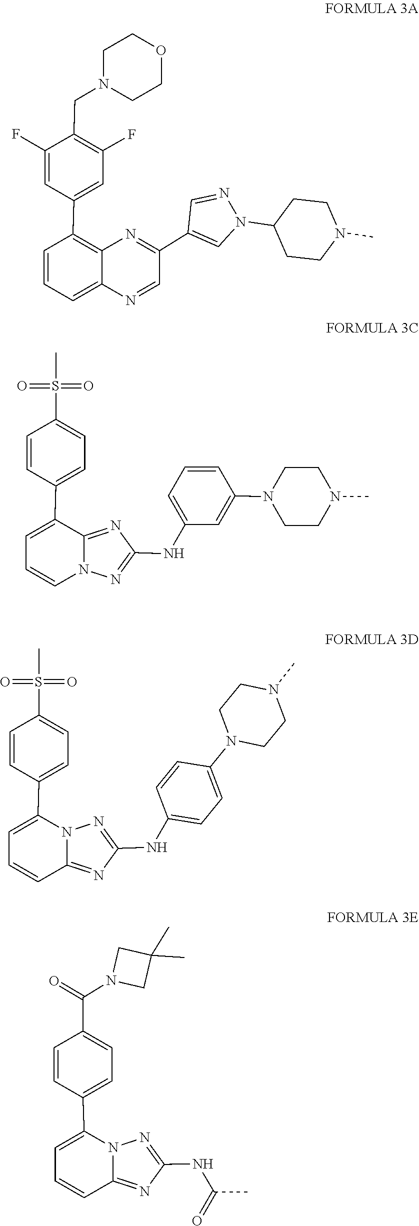

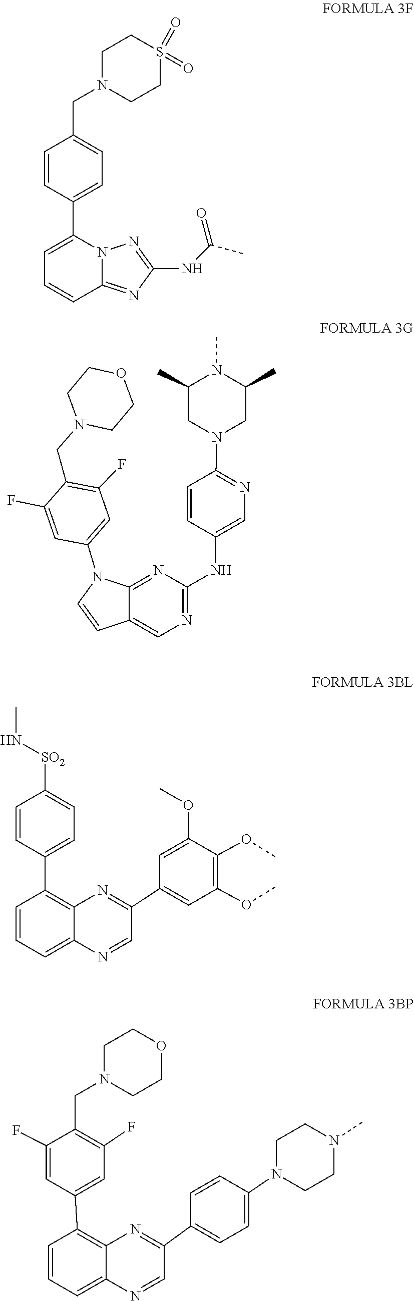

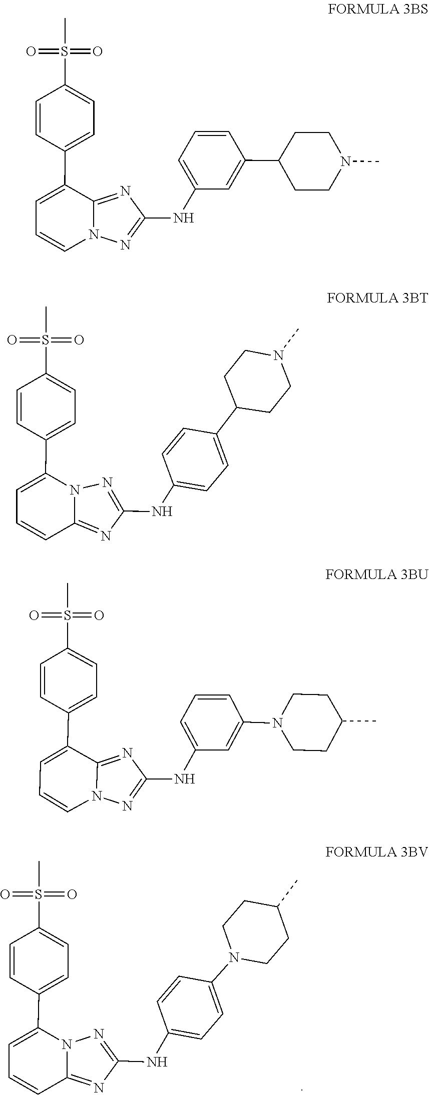

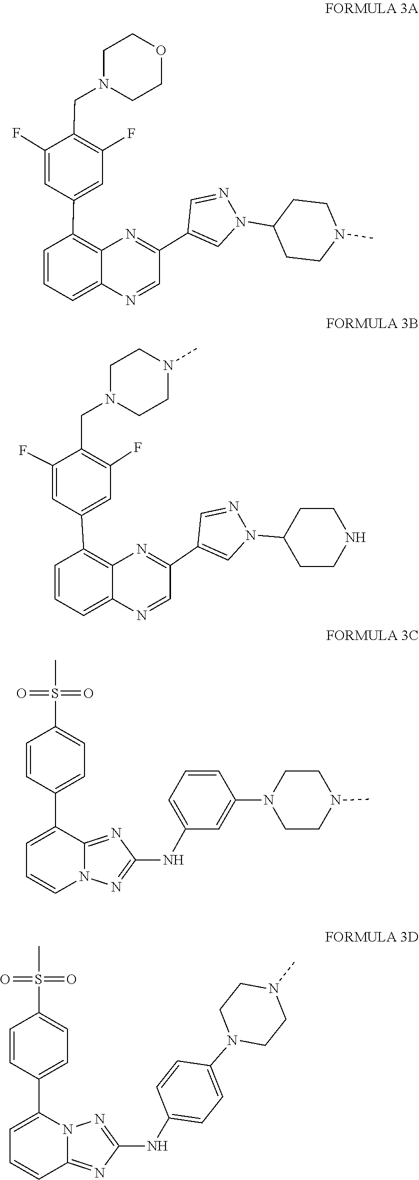

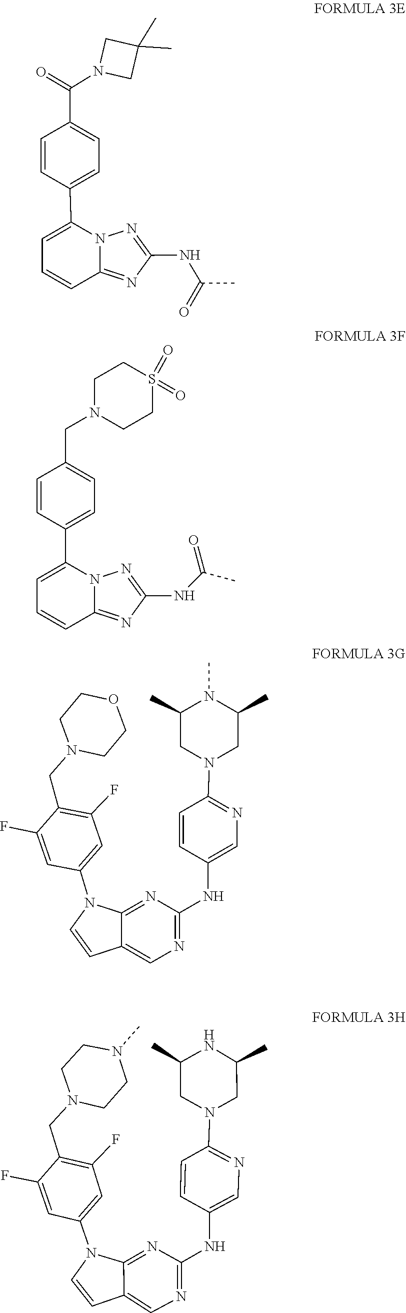

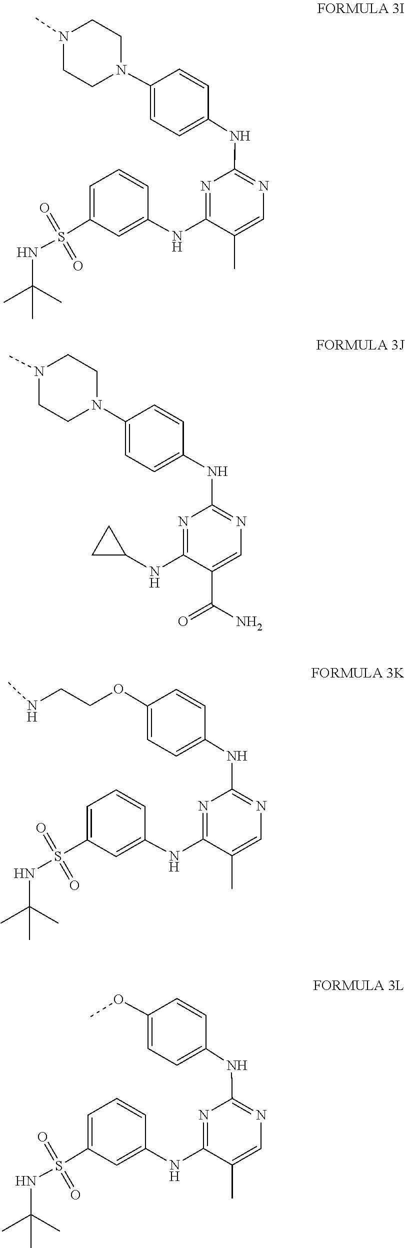

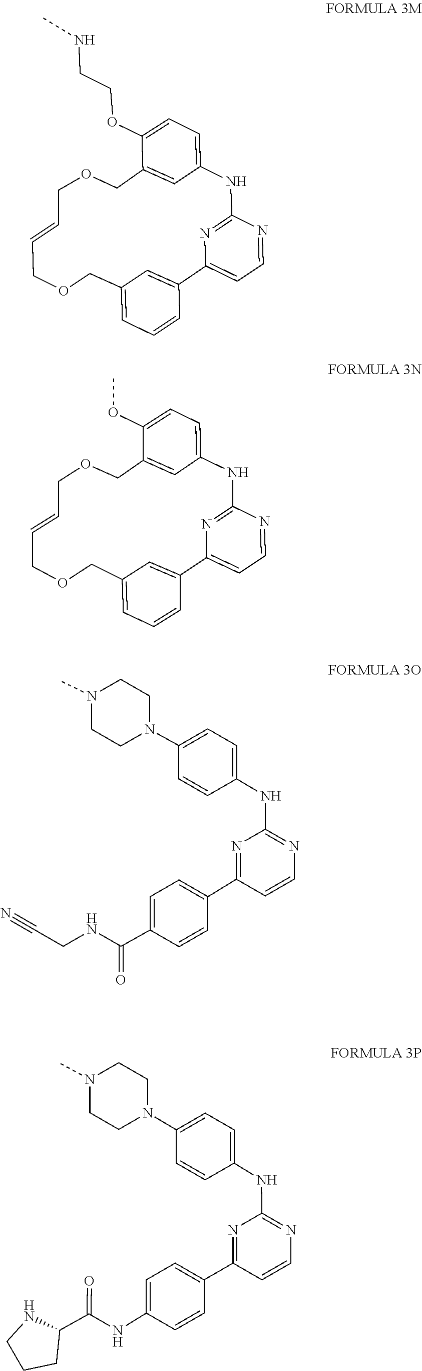

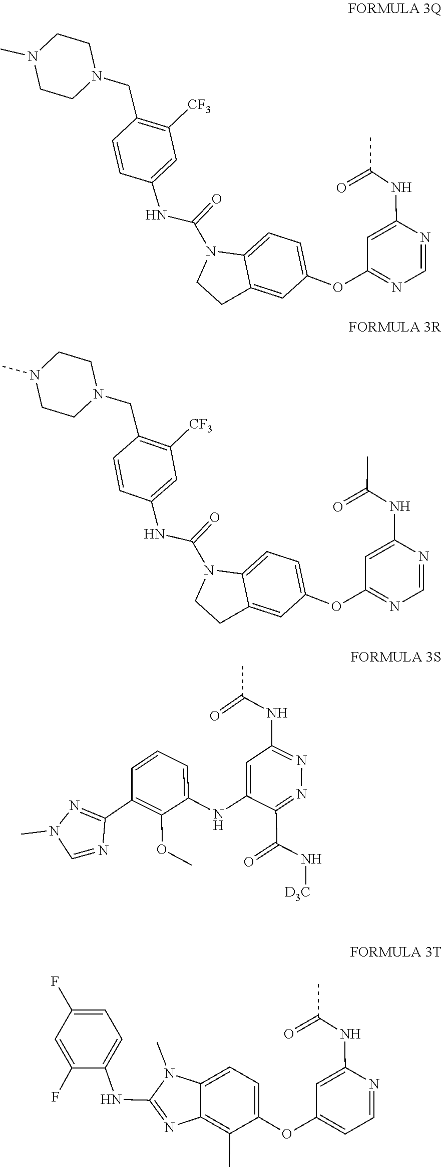

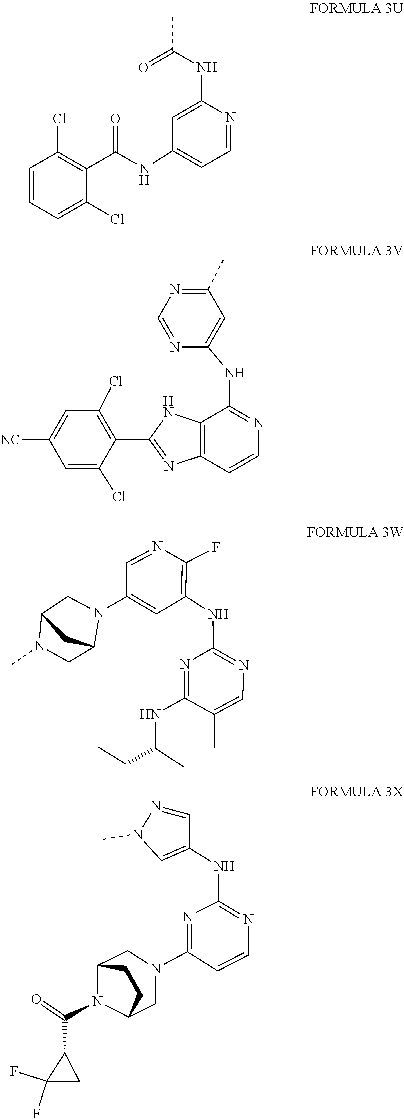

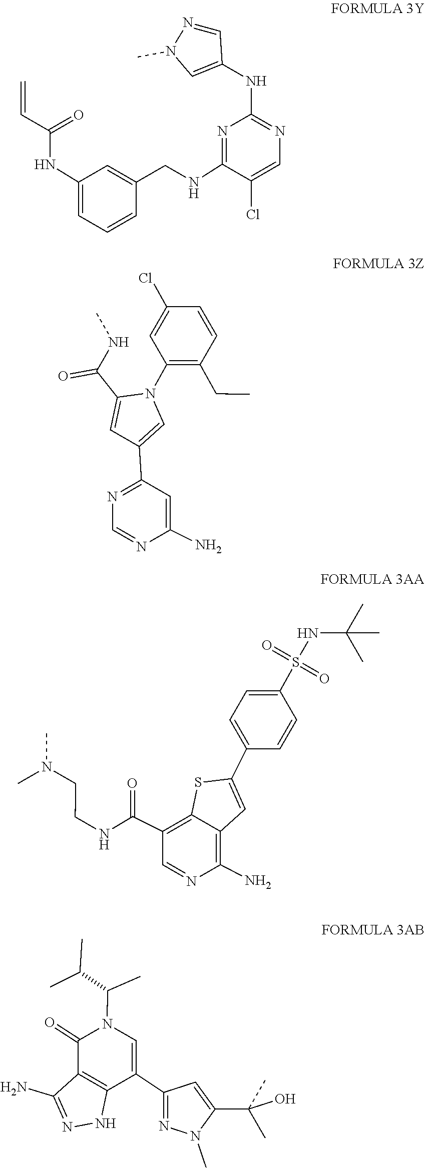

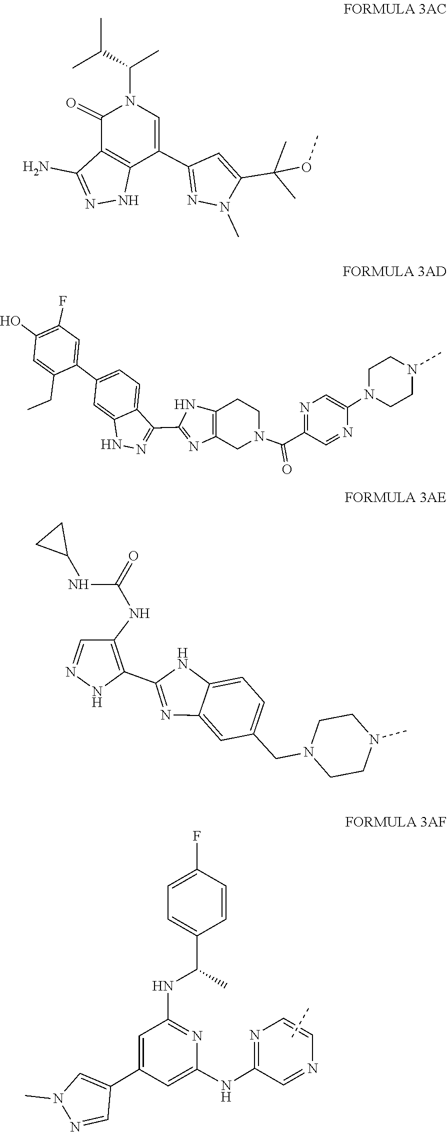

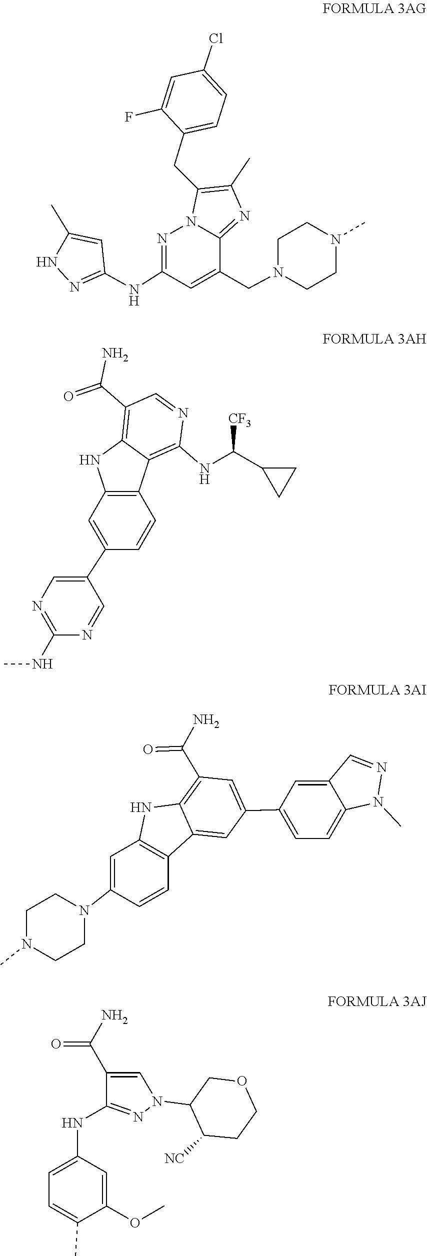

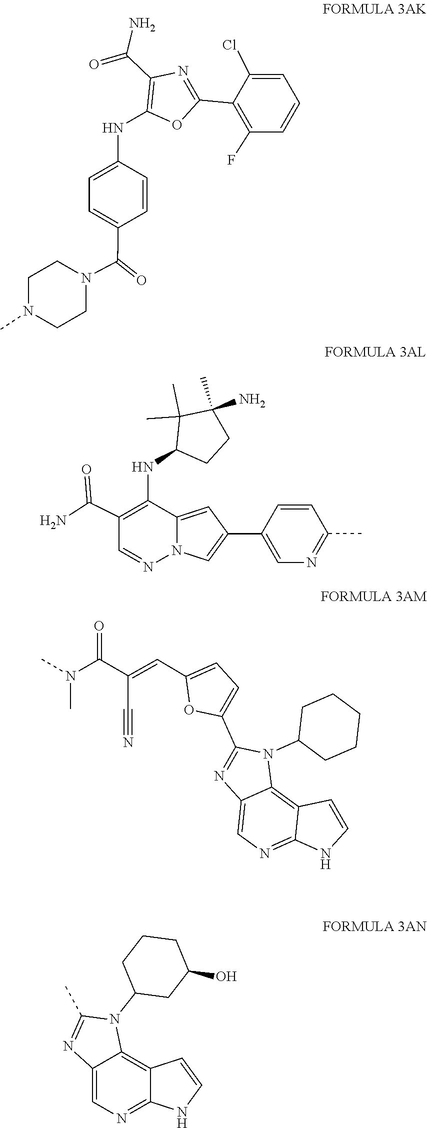

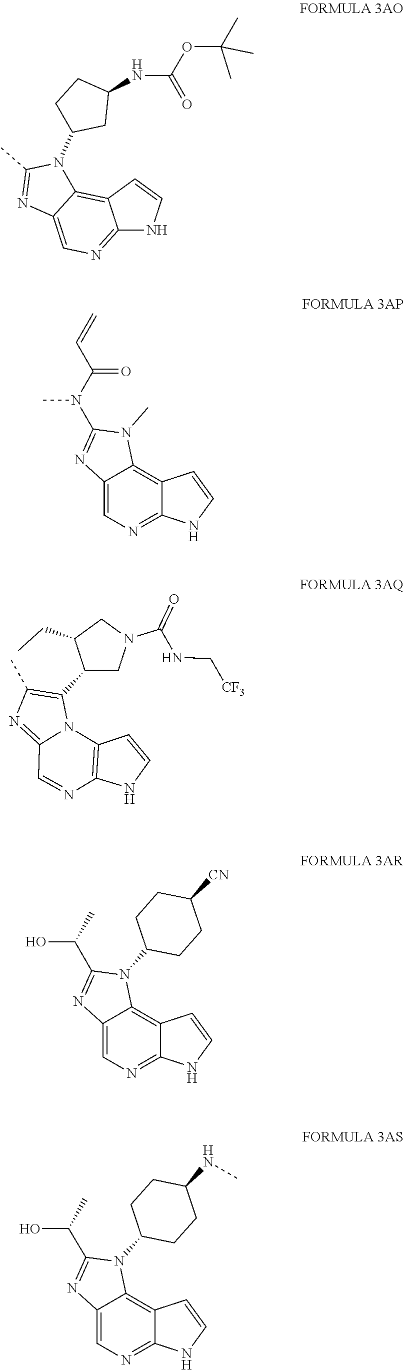

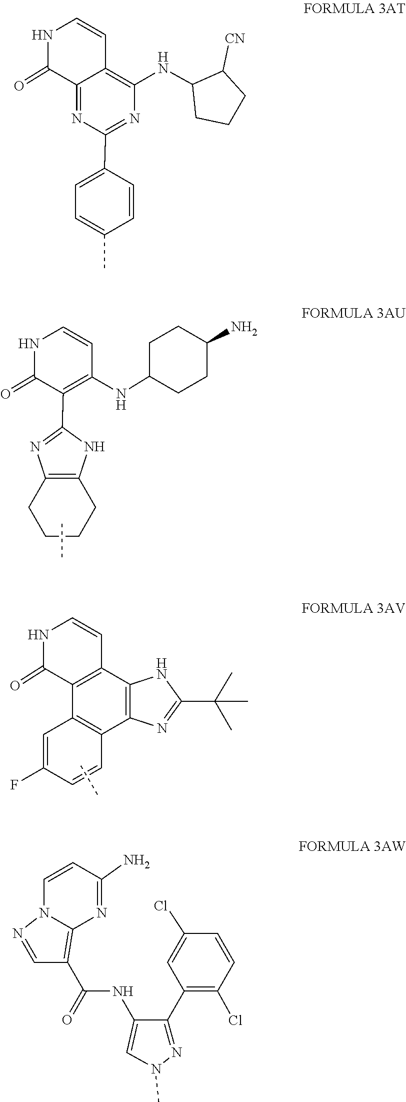

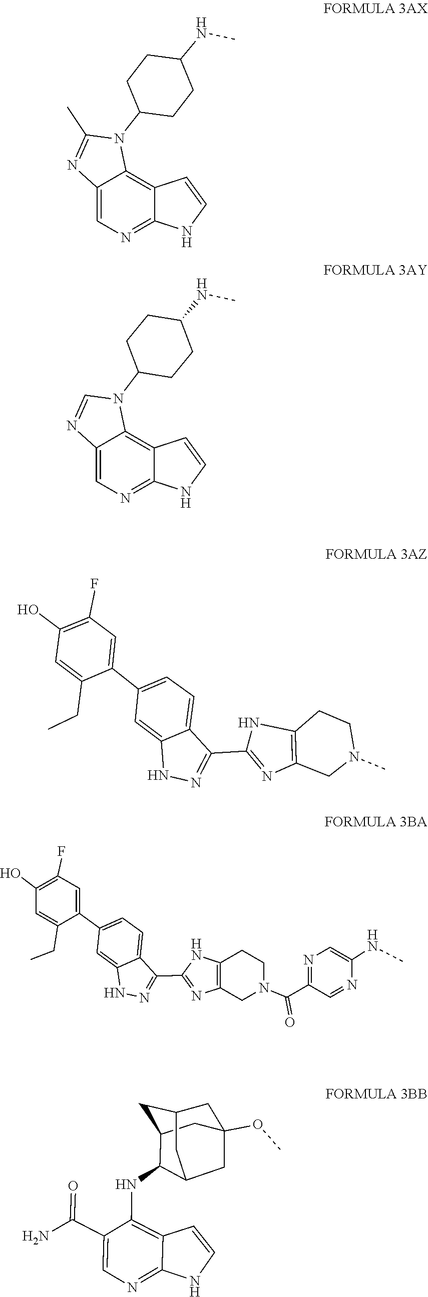

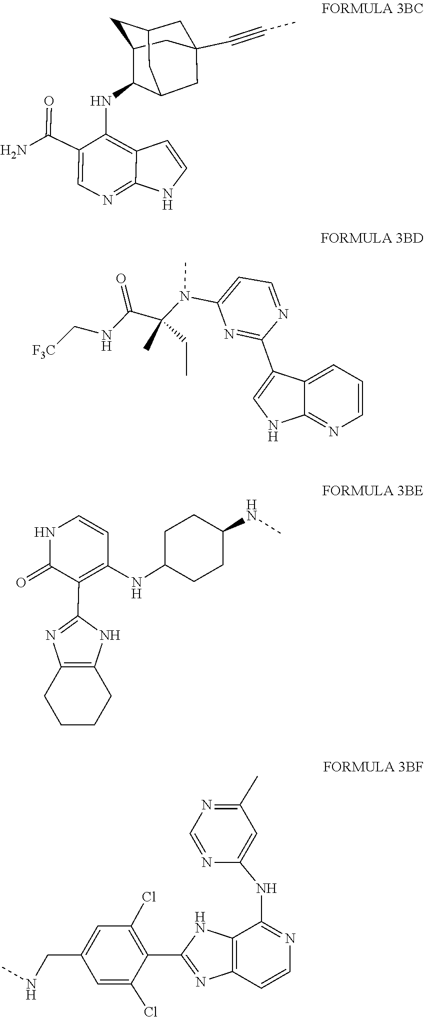

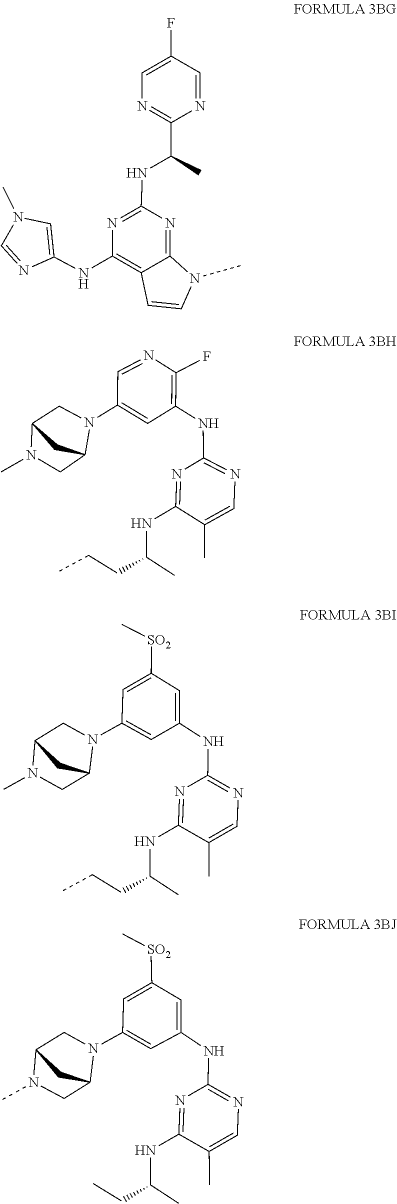

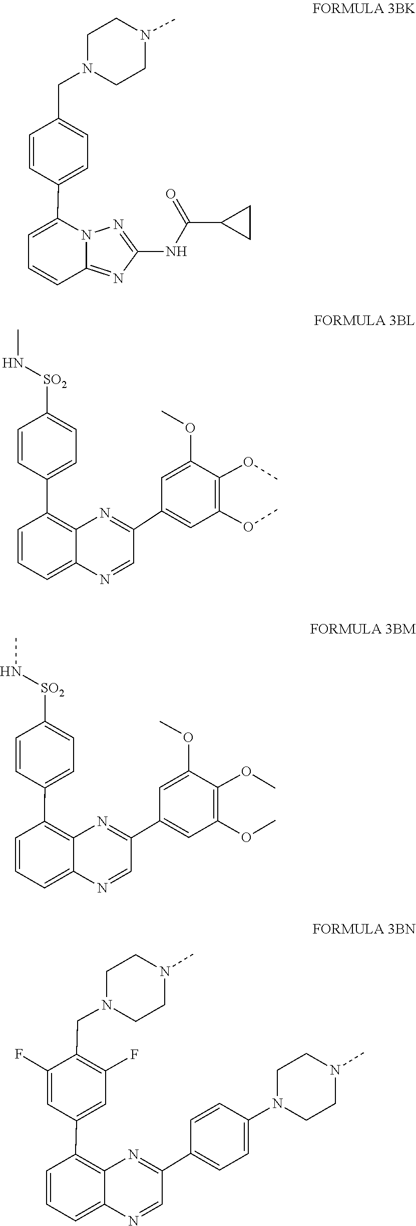

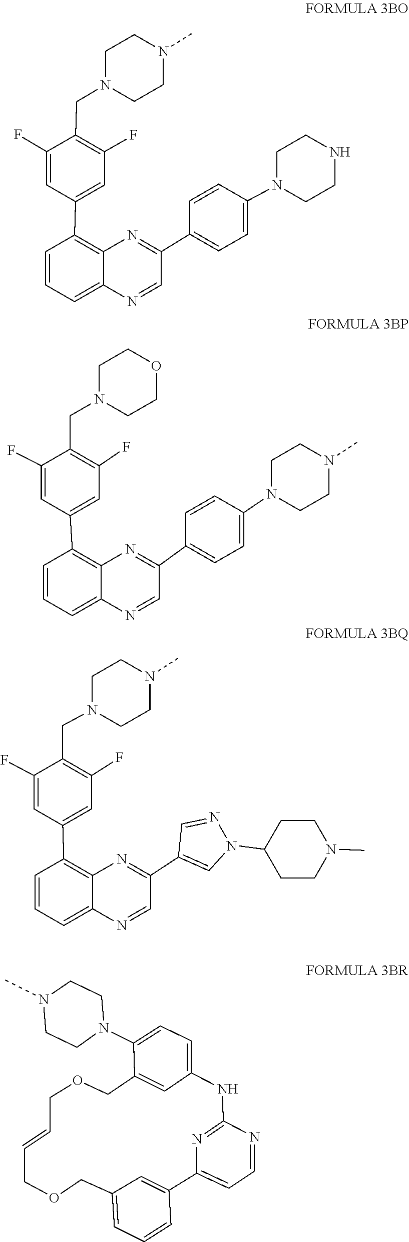

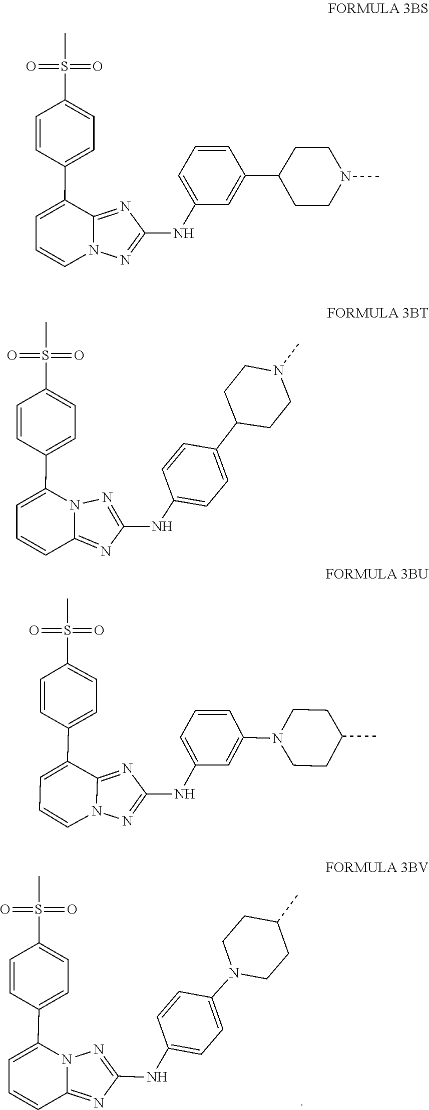

57. The heterobifunctional compound of claim 1, wherein
(II-1) the degradation tag is a moiety selected from the group consisting of FORMULAE 5A, 5B, 5C, and 5D: 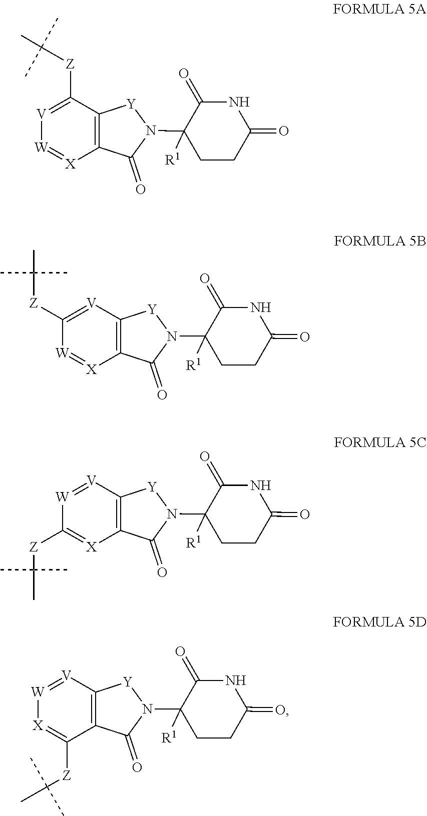
wherein
V, W, and X are independently selected from CR² and N;
Y is selected from —CO—, —CR³R⁴—, —N═CR³—, and —N═N—;
Z is selected from null, CO, CR⁵R⁶, NR⁵, O, C≡C, optionally substituted C₁-C₁₀ alkylene, optionally substituted C₂-C₁₀ alkenyl, and optionally substituted C₂-C₁₀ alkynyl, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R¹, R², R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl, or
R³ and R⁴ together with the atom to which they are connected form a 3-6 membered carbocyclyl, or 4-6 membered heterocyclyl; and
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl, or R⁵ and R⁶ together with the atom to which they are connected form a 3-6 membered carbocyclyl, or 4-6 membered heterocyclyl;
or
(II-2) the degradation tag is a moiety selected from the group consisting of FORMULAE 5E, 5F, 5G, 5H, 5I, 5J, 5K, 5L, 5M, 5N, 5O, 5P, and 5: 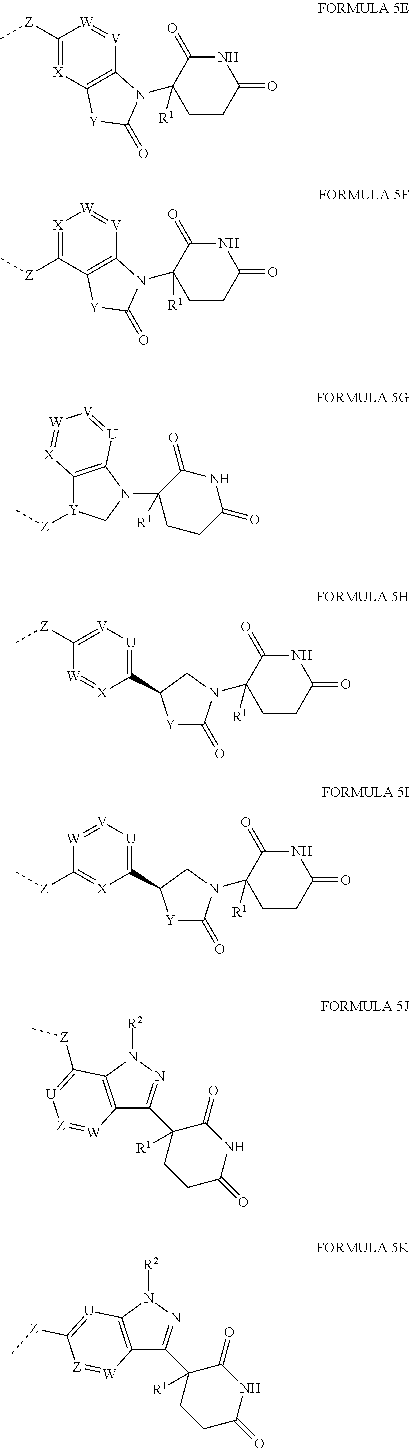 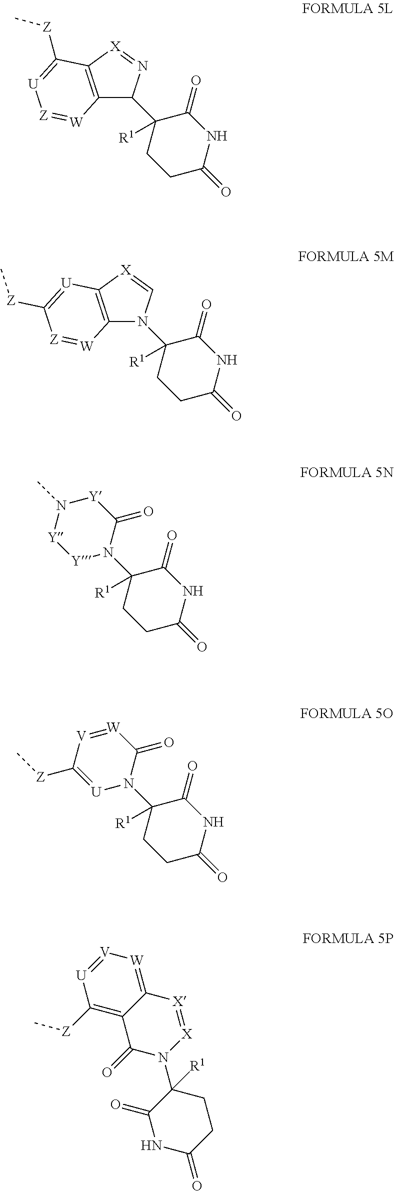 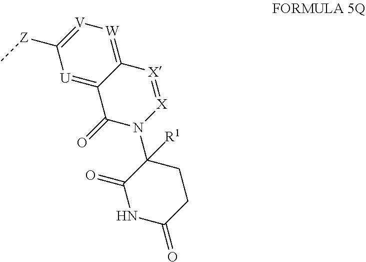
wherein
U, V, W, X, and X′ are independently selected from CR² and N;
Y is selected from —N—, —CR³═, —CR³R⁴—, —NR³— and —O—; preferably, Y is selected from —N—, —CH₂—, —NH—, —N(CH₃)— and —O—;
Y′, Y″, and Y′″ are independently selected from CR³R⁴;
Z is selected from null, CO, CR⁵R⁶, NR⁵, O, optionally substituted C₁-C₁₀ alkylene, optionally substituted C₁-C₁₀ alkenylene, optionally substituted C₁-C₁₀ alkynylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl; preferably, Z is selected from null, CH₂, CH═CH, C≡C, NH and O;
R¹, and R² are independently selected from hydrogen, halogen, cyano, nitro, optionally substituted C₁-C₆ alkyl, optionally substituted 3 to 6 membered carbocyclyl, and optionally substituted 4 to 6 membered heterocyclyl;
R³, and R⁴ are independently selected from hydrogen, halogen, cyano, nitro, optionally substituted C₁-C₆ alkyl, optionally substituted 3 to 6 membered carbocyclyl, and optionally substituted 4 to 6 membered heterocyclyl; or R³ and R⁴ together with the atom to which they are connected form a 3-6 membered carbocyclyl, or 4-6 membered heterocyclyl;
R⁵ and R⁶ are independently selected from null, hydrogen, halogen, oxo, hydroxyl, amino, cyano, nitro, optionally substituted C₁-C₆ alkyl, optionally substituted 3 to 6 membered carbocyclyl, and optionally substituted 4 to 6 membered heterocyclyl; or R⁵ and R⁶ together with the atom to which they are connected form a 3-6 membered carbocyclyl, or 4-6 membered heterocyclyl; and
R′ is selected from hydrogen, optionally substituted C₁-C₆ alkyl, optionally substituted 3 to 6 membered carbocyclyl, and optionally substituted 4 to 6 membered heterocyclyl;
or
(II-3) the degradation tag is a moiety of FORMULA 6A: 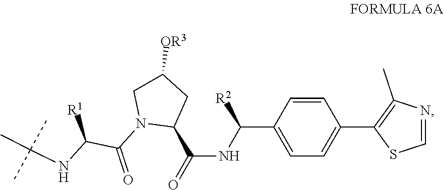
wherein
R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl; and
R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-C₈alkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-C₈alkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂;
or
(II-4) the degradation tag is a moiety of FORMULAE 6B, 6C, and 6D: 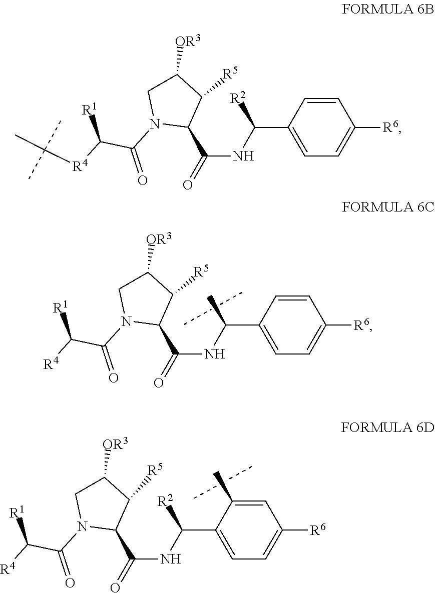
wherein
R¹ and R² are independently selected from hydrogen, hydroxyl, amino, cyano, nitro, optionally substituted C₁-C₈ alkyl, optionally substituted C₂-C₈ alkenyl, optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈ aminoalkyl, optionally substituted C₁-C₈alkylaminoC₁-C₈alkyl, optionally substituted 3-10 membered carbocyclyl, and optionally substituted 4-10 membered heterocyclyl;
R³ is selected from hydrogen, optionally substituted C(O)C₁-C₈ alkyl, optionally substituted C(O)C₁-C₈alkoxyC₁-C₈alkyl, optionally substituted C(O)C₁-C₈ haloalkyl, optionally substituted C(O)C₁-C₈ hydroxyalkyl, optionally substituted C(O)C₁-C₈ aminoalkyl, optionally substituted C(O)C₁-C₈alkylaminoC₁-C₈alkyl, optionally substituted C(O)(3-10 membered carbocyclyl), optionally substituted C(O)(4-10 membered heterocyclyl), optionally substituted C(O)C₂-C₈ alkenyl, optionally substituted C(O)C₂-C₈ alkynyl, optionally substituted C(O)OC₁-C₈alkoxyC₁-C₈alkyl, optionally substituted C(O)OC₁-C₈ haloalkyl, optionally substituted C(O)OC₁-C₈ hydroxyalkyl, optionally substituted C(O)OC₁-C₈ aminoalkyl, optionally substituted C(O)OC₁-CsalkylaminoC₁-C₈alkyl, optionally substituted C(O)O(3-10 membered carbocyclyl), optionally substituted C(O)O(4-10 membered heterocyclyl), optionally substituted C(O)OC₂-C₈ alkenyl, optionally substituted C(O)OC₂-C₈ alkynyl, optionally substituted C(O)NC₁-C₈alkoxyC₁-C₈alkyl, optionally substituted C(O)NC₁-C₈ haloalkyl, optionally substituted C(O)NC₁-C₈ hydroxyalkyl, optionally substituted C(O)NC₁-C₈ aminoalkyl, optionally substituted C(O)NC₁-CsalkylaminoC₁-C₈alkyl, optionally substituted C(O)N(3-10 membered carbocyclyl), optionally substituted C(O)N(4-10 membered heterocyclyl), optionally substituted C(O)NC₂-C₈ alkenyl, optionally substituted C(O)NC₂-C₈ alkynyl, optionally substituted P(O)(OH)₂, optionally substituted P(O)(OC₁-C₈ alkyl)₂, and optionally substituted P(O)(OC₁-C₈ aryl)₂, and
R⁴ is selected from NR⁷R⁸, 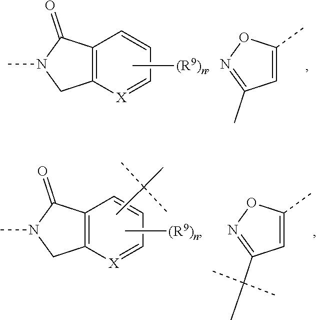
optionally substituted C₁-C₈alkoxy, optionally substituted 3-10 membered carbocyclyl, optionally substituted 4-10 membered heterocyclyl, optionally substituted aryl, and optionally substituted heteraryl, in which
R⁷ is selected from null, hydrogen, optionally substituted C₁-C₈alkyl, optionally substituted C₁-C₈cycloalkyl, optionally substituted C₁-C₈alkyl-CO, optionally substituted C₁-C₈cycloalkyl-CO, optionally substituted C₁-C₈cycloalkyl-C₁-C₈alkyl-CO, optionally substituted 4-10 membered heterocyclyl-CO, optionally substituted 4-10 membered heterocyclyl-C₁-C₈alkyl-CO, optionally substituted aryl-CO, optionally substituted aryl-C₁-C₈alkyl-CO, optionally substituted heteroaryl-CO, optionally substituted heteroaryl-C₁-C₈alkyl-CO, optionally substituted aryl, and optionally substituted heteroaryl;
R⁸ is selected from null, hydrogen, optionally substituted C₁-C₈alkyl, and optionally substituted C₁-C₈cycloalkyl;
R⁹, at each occurrence, is independently selected from hydrogen, halogen, cyano, optionally substituted C₁-C₈alkyl, optionally substituted C₁-C₈cycloalkyl, optionally substituted C₁-C₈heterocycloalkyl, optionally substituted C₁-C₈alkoxy, optionally substituted C₁-C₈cycloalkoxy, halo substituted C₁-C₈alkyl, halo substituted C₁-C₈cycloalkyl, halo substituted C₁-C₈alkoxl, halo substituted C₁-C₈cycloalkoxy, and halo substituted C₁-C₈heterocycloalkyl;
X is selected from CH and N; and
n is 0, 1, 2, 3, or 4;
R⁶ is selected from hydrogen, halogen, hydroxyl, amino, cyano, nitro, optionally substituted C₁-C₈alkyl, optionally substituted C₁-C₈cycloalkyl, optionally substituted C₁-C₈alkoxy, and optionally substituted C₁-C₈cycloalkoxy, optionally substituted C₁-C₈heterocycloalkyl, optionally substituted aryl, and optionally substituted heteroaryl, preferably, halogen, cyano, optionally substituted imidazole, optionally substituted pyrazole, optionally substituted oxadiazole, optionally substituted triazole, 4-methylthiazol-5-yl, or oxazol-5-yl group;
or
(II-5) the degradation tag is a moiety of FORMULA 7A: 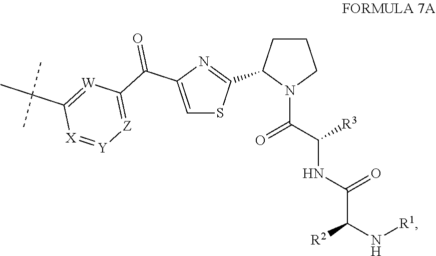
wherein
V, W, X, and Z are independently selected from CR⁴ and N; and
R¹, R², R³, and R⁴ are independently selected from hydrogen, halogen, cyano, nitro, optionally substituted C₁-C₈ alkyl, optionally substituted C₂-C₈ alkenyl, and optionally substituted C₂-C₈ alkynyl; optionally substituted C₁-C₈alkoxyC₁-C₈alkyl, optionally substituted C₁-C₈ haloalkyl, optionally substituted C₁-C₈ hydroxyalkyl, optionally substituted C₁-C₈alkoxy, optionally substituted C₁-C₈alkylamino, optionally substituted 3-10 membered carbocyclyl, and optionally substituted 4-10 membered heterocyclyl;
or
(II-6) the degradation tag is a moiety of FORMULA 7B: 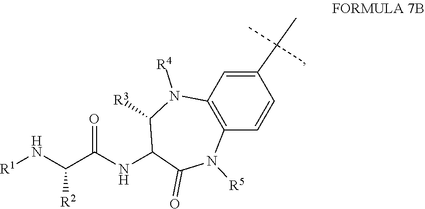
wherein
R¹, R², and R³ are independently selected from hydrogen, halogene, optionally substituted C₁-C₈ alkyl, optionally substituted C₁-C₈alkoxyC₁-C₈alkyl, optionally substituted C₁-C₈ haloalkyl, optionally substituted C₁-C₈ hydroxyalkyl, optionally substituted C₃-C₇ cycloalkyl, optionally substituted 3-7 membered heterocyclyl, optionally substituted C₂-C₈ alkenyl, and optionally substituted C₂-C₈ alkynyl;
R⁴ and R⁵ are independently selected from hydrogen, COR⁶, CO₂R⁶, CONR⁶R⁷, SOR⁶, SO₂R⁶, SO₂NR⁶R⁷, optionally substituted C₁-C₈ alkyl, optionally substituted C₁-C₈alkoxyC₁-C₈alkyl, optionally substituted C₁-C₈alkylaminoC₁-C₈alkyl, optionally substituted aryl-C₁-C₈alkyl, optionally substituted 3-8 membered cycloalkyl, optionally substituted 3-8 membered heterocyclyl, optionally substituted aryl, and optionally substituted heteroaryl, wherein
R⁶ and R⁷ are independently selected from hydrogen, optionally substituted C₁-C₈ alkyl, optionally substituted C₁-C₈alkoxyC₁-C₈alkyl, optionally substituted C₁-C₈alkylaminoC₁-C₈alkyl, optionally substituted 3-8 membered cycloalkyl, optionally substituted 3-8 membered heterocyclyl, optionally substituted aryl, and optionally substituted heteroaryl, or
R⁶ and R⁷ together with the atom to which they are connected form a 4-8 membered cycloalkyl or heterocyclyl ring.

58. The heterobifunctional compound claim 57, wherein in FORMULAE 5A, 5B, 5C, or 5D, Y is selected from —CO—, —CH₂—, and —N═N—.

59-63. (canceled)

64. The heterobifunctional compound of claim 1, wherein the degradation tag is derived from any of the following:

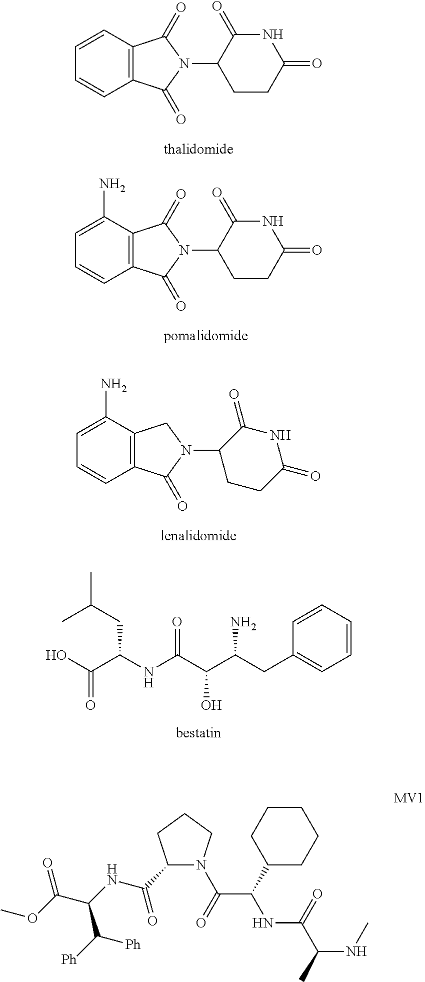

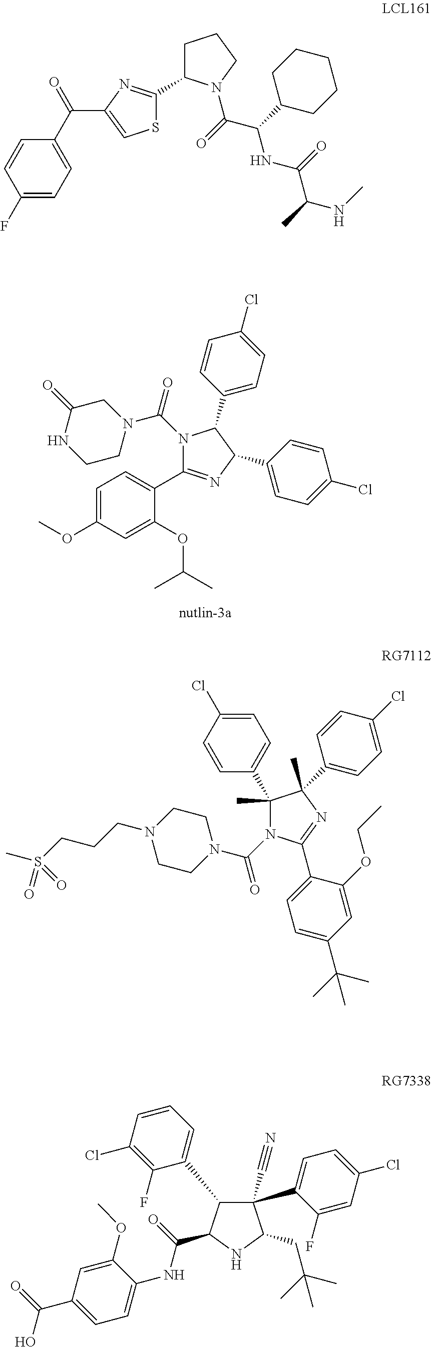

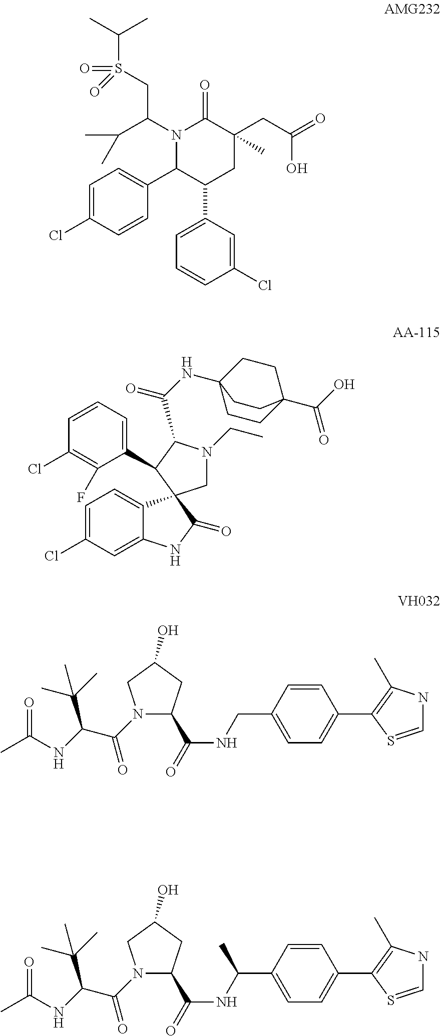

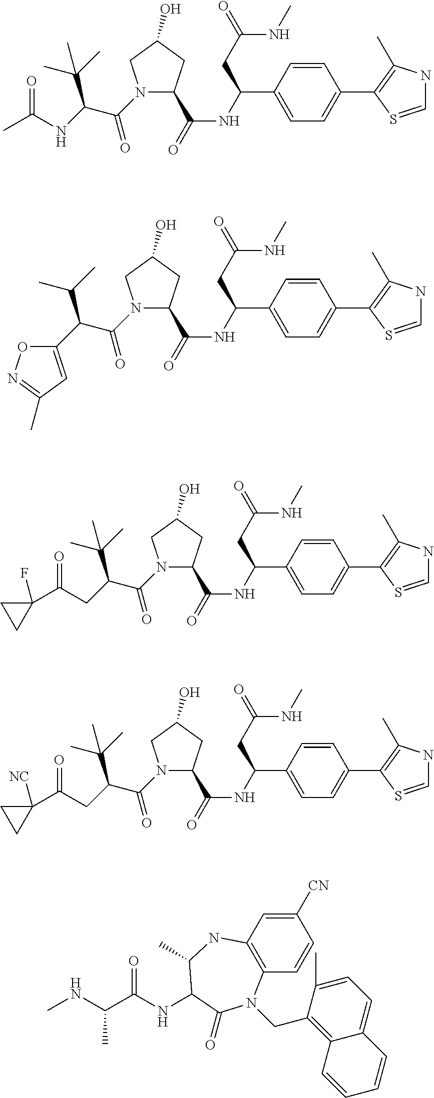

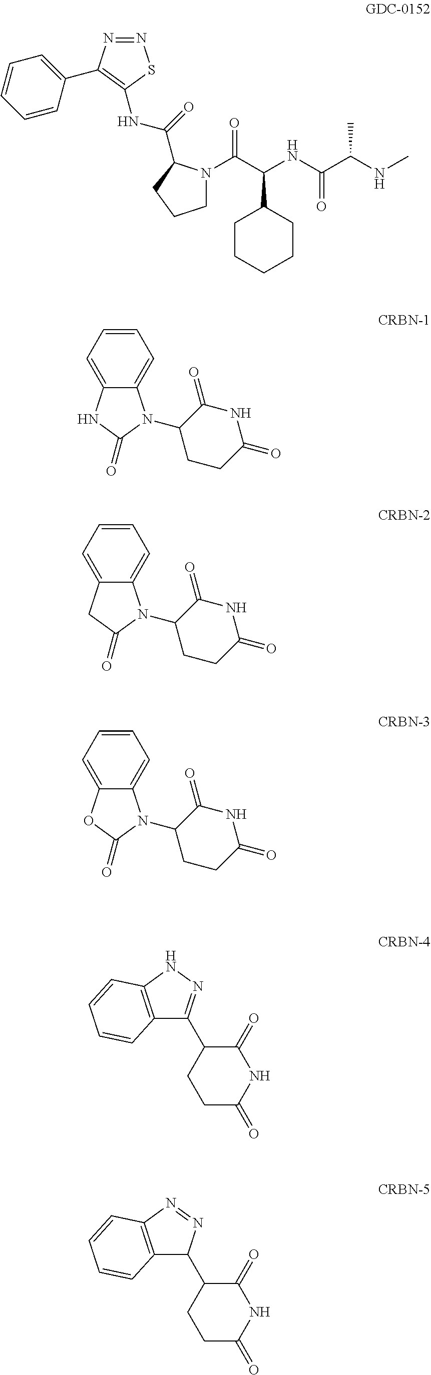 [SMILES: CN[C@@H](C)C(=O)N[C@H](C(=O)N1CCC[C@H]1C(=O)Nc1snnc1-c1ccccc1)C1CCCCC1 ; O=C1CCC(n2c(=O)[nH]c3ccccc32)C(=O)N1 ; O=C1CCC(N2C(=O)Cc3ccccc32)C(=O)N1 ; O=C1CCC(n2c(=O)oc3ccccc32)C(=O)N1 ; O=C1CCC(c2n[nH]c3ccccc23)C(=O)N1 ; O=C1CCC(C2N=Nc3ccccc32)C(=O)N1]

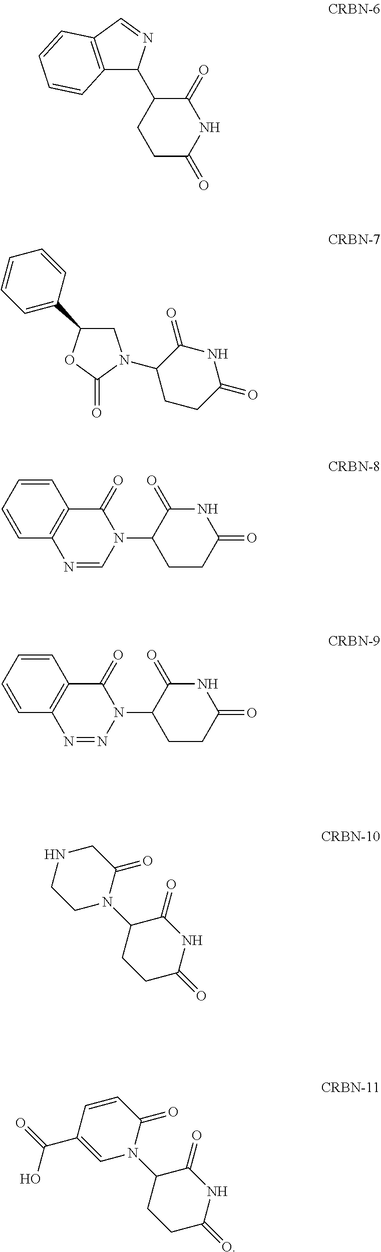 [SMILES: O=C1CCC(C2N=Cc3ccccc32)C(=O)N1 ; O=C1CCC(N2C[C@H](c3ccccc3)OC2=O)C(=O)N1 ; O=C1CCC(n2cnc3ccccc3c2=O)C(=O)N1 ; O=C1CCC(n2nnc3ccccc3c2=O)C(=O)N1 ; O=C1CCC(N2CCNCC2=O)C(=O)N1 ; O=C1CCC(n2cc(C(=O)O)ccc2=O)C(=O)N1]

65. The heterobifunctional compound of claim 1, wherein the degradation tag is selected from the group consisting of:

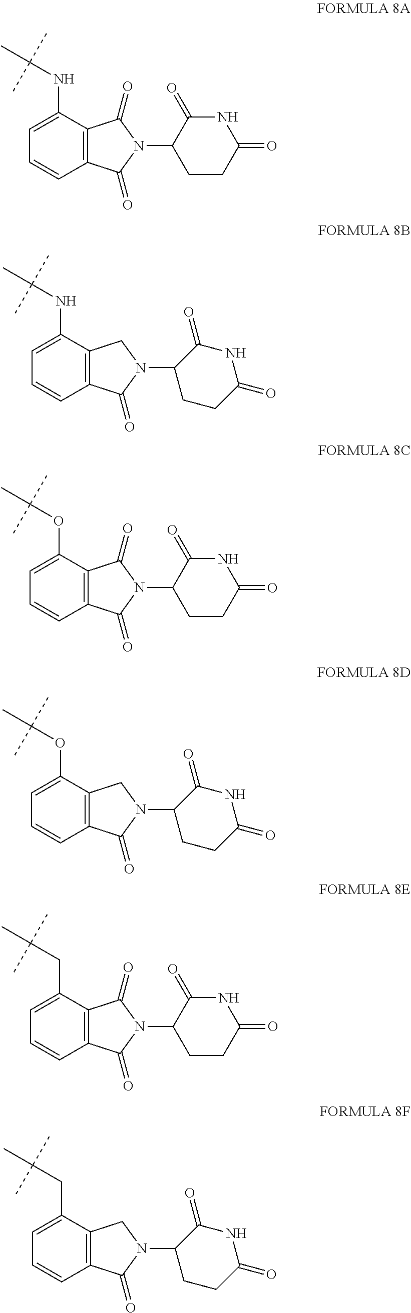

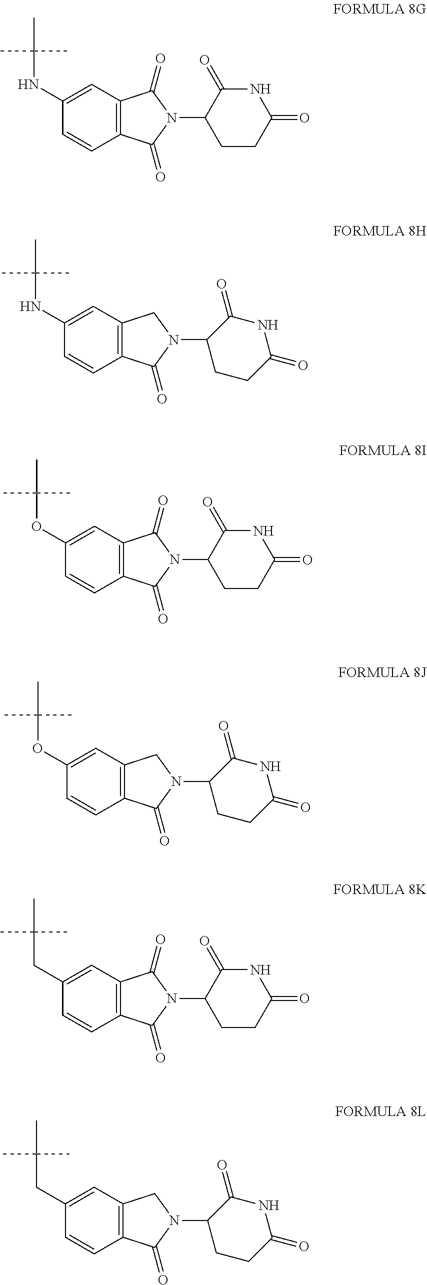

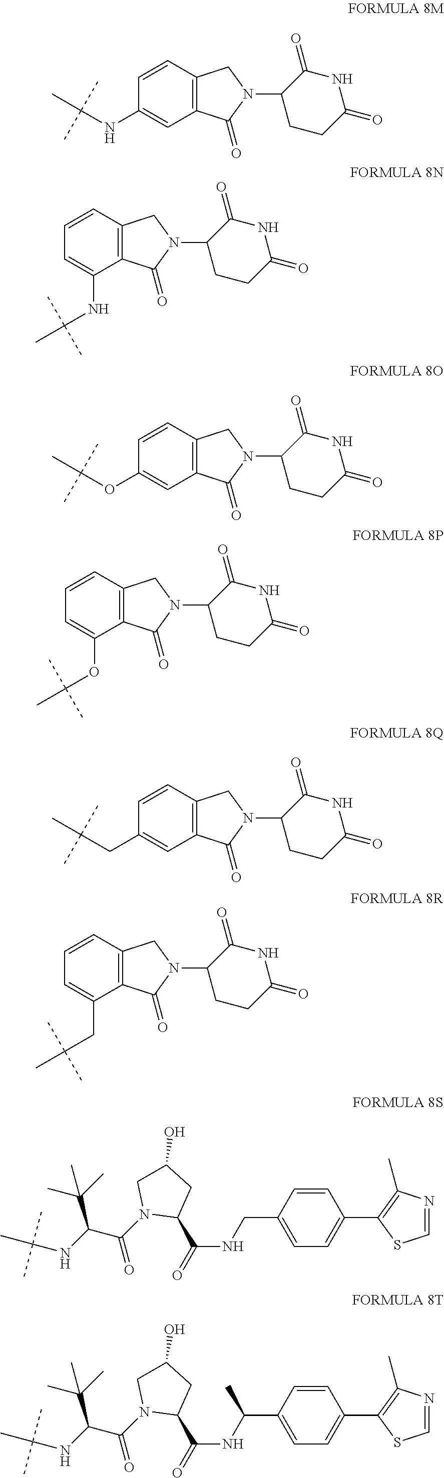

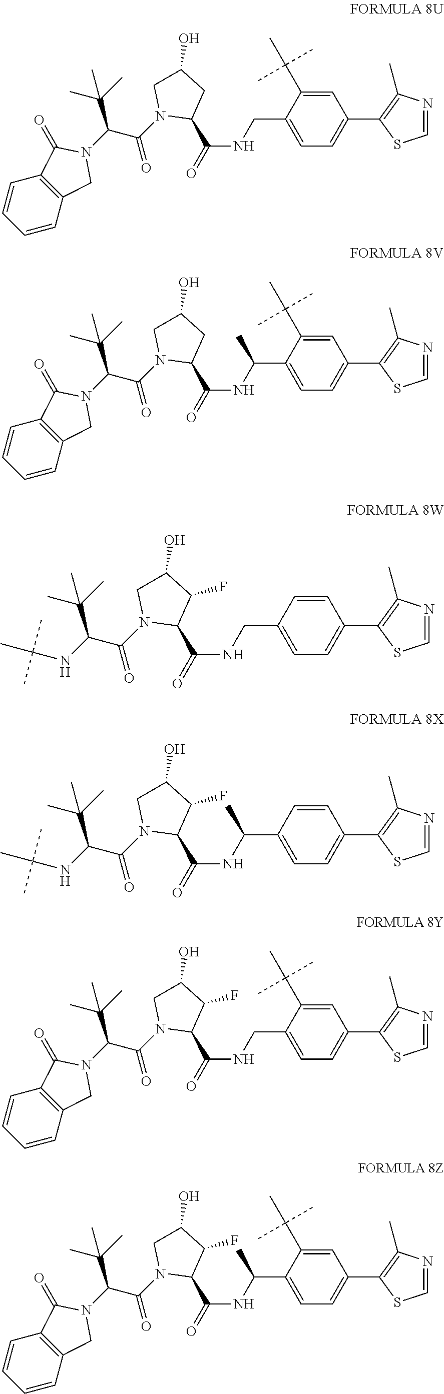

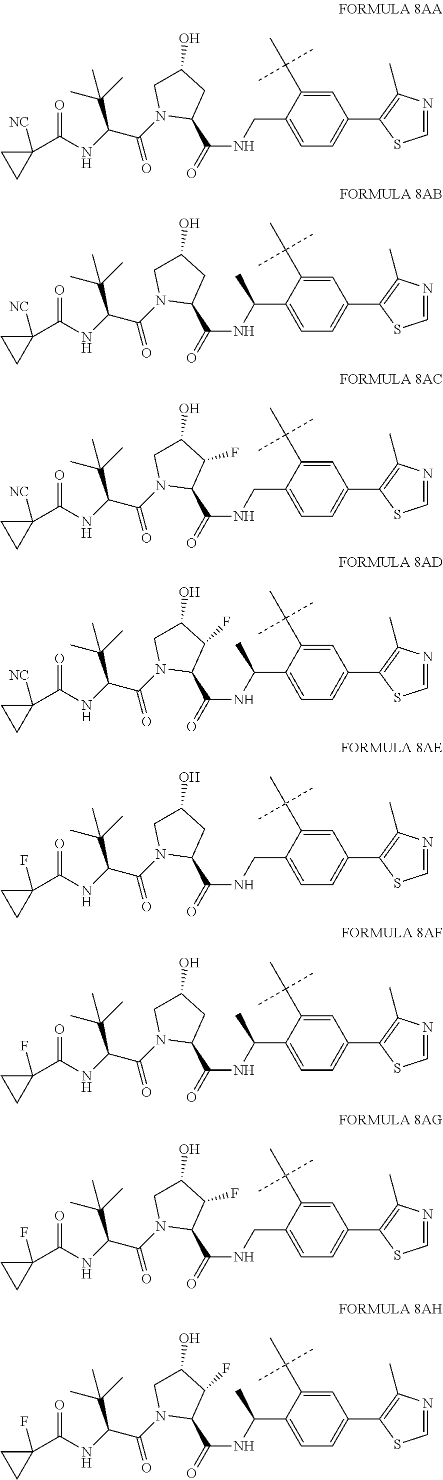

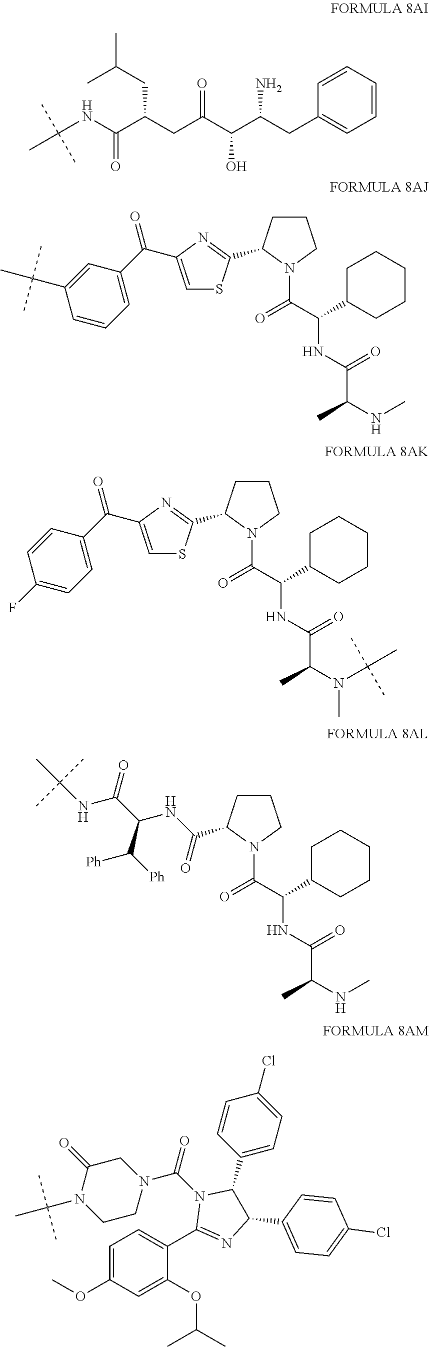

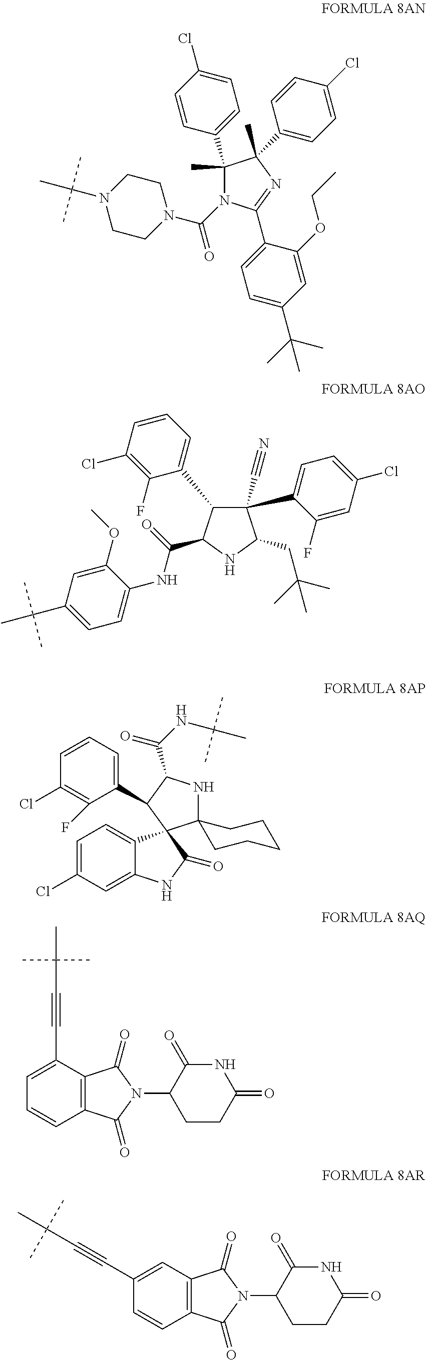

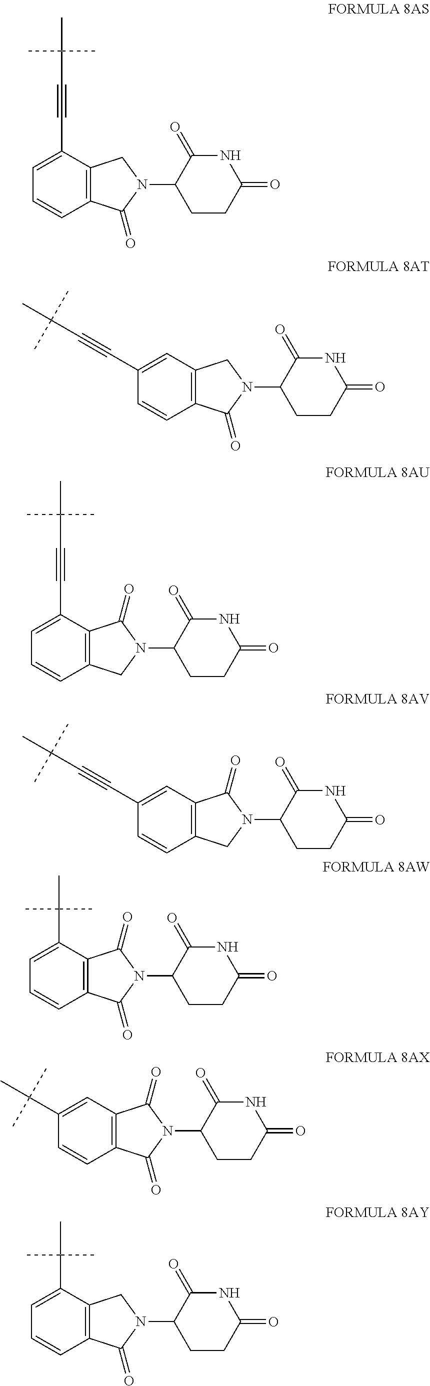

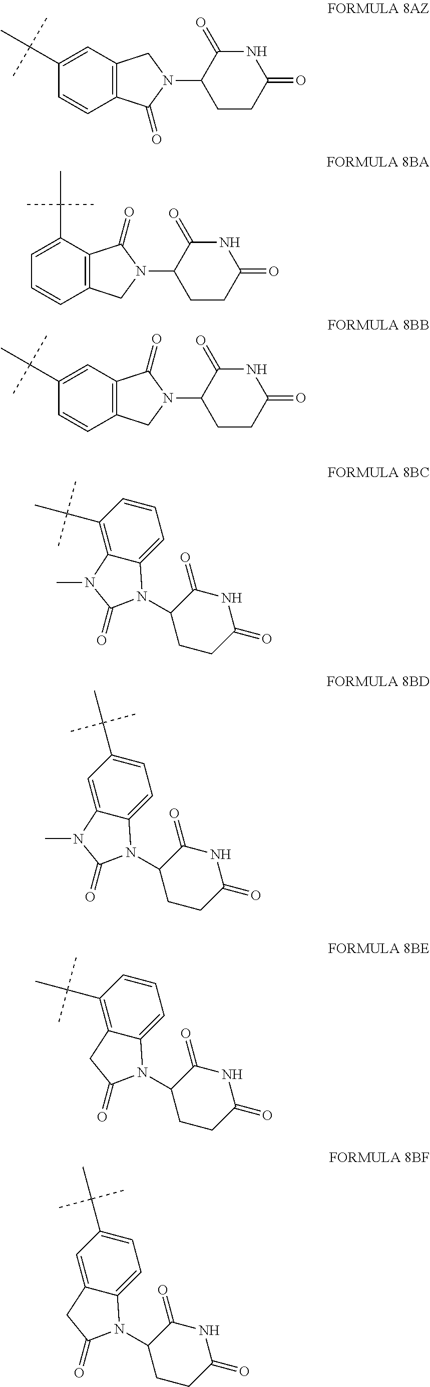

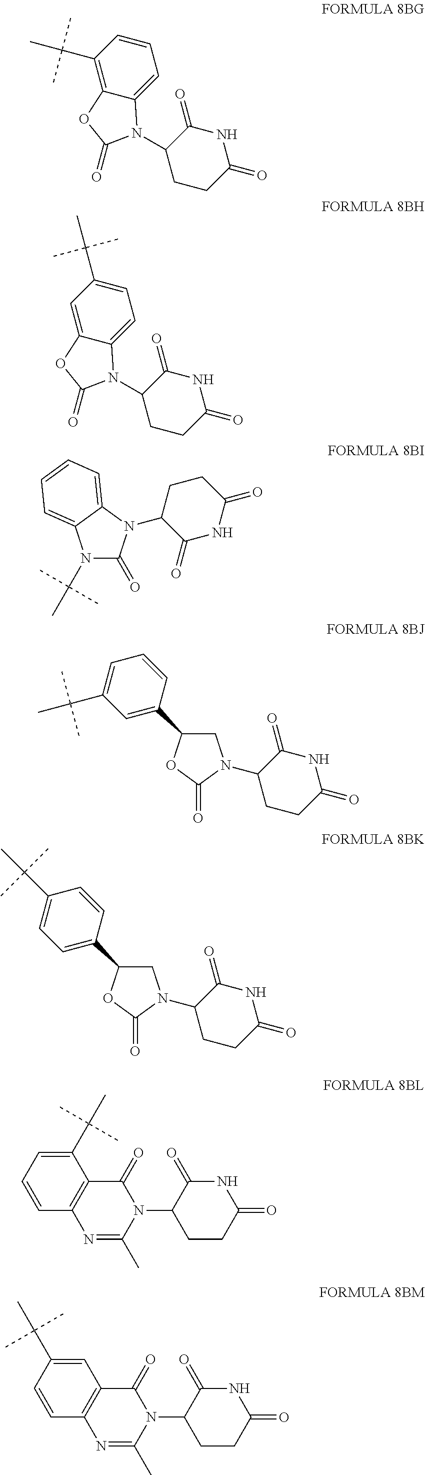

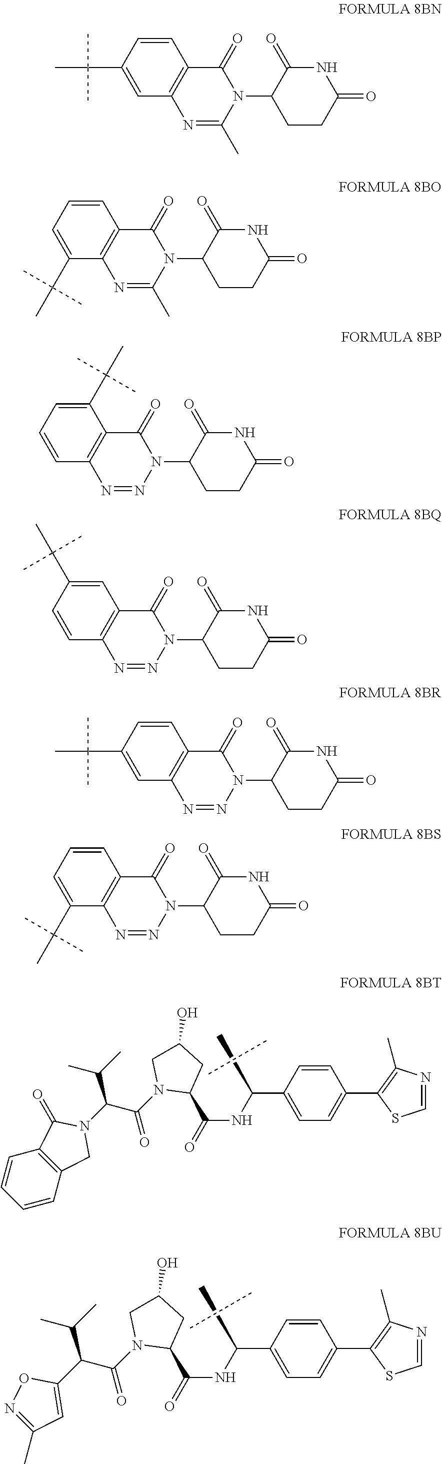

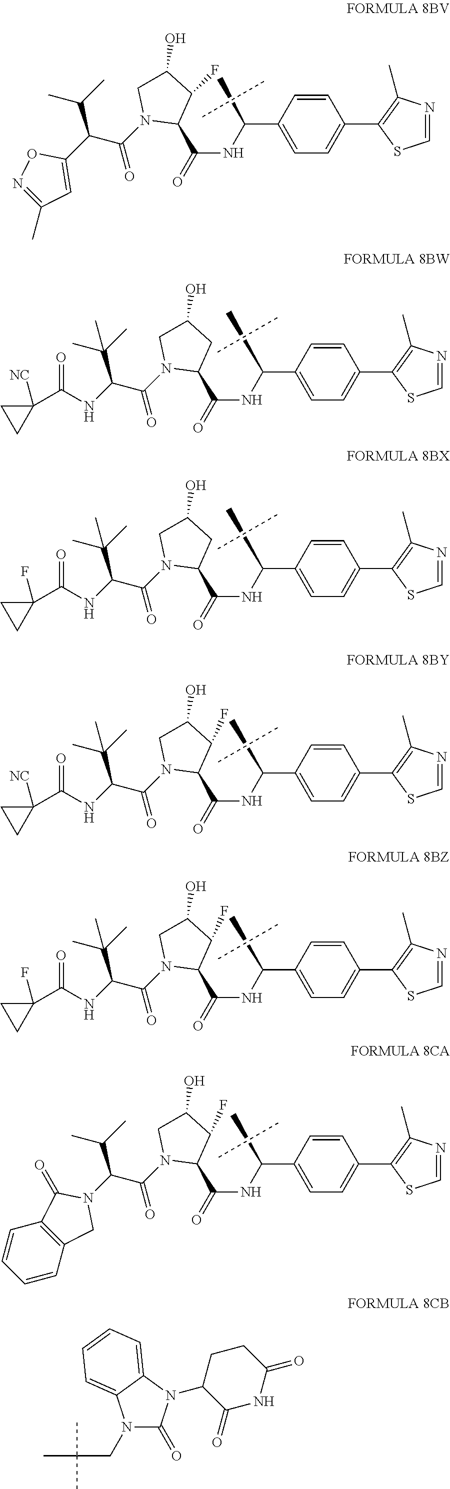

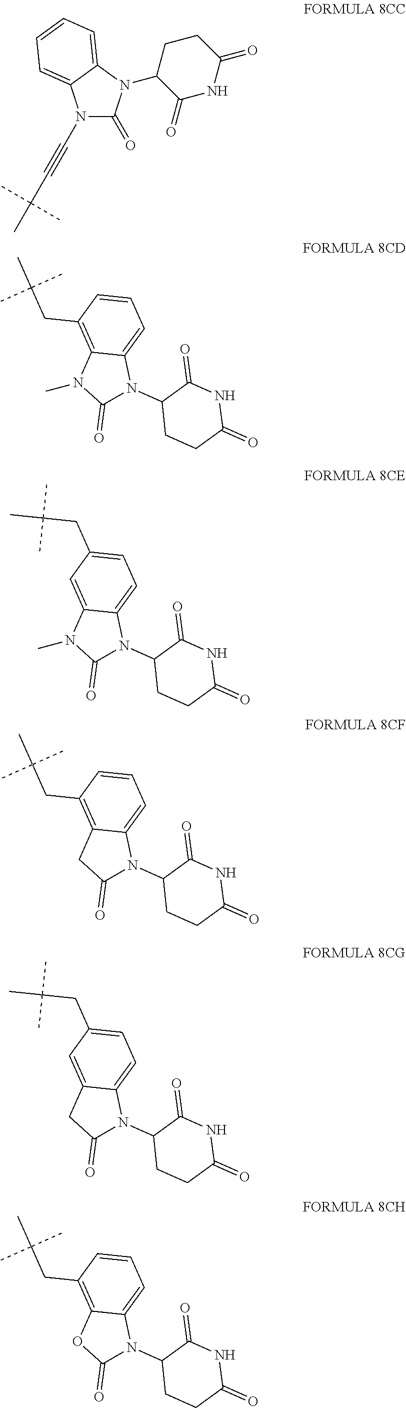

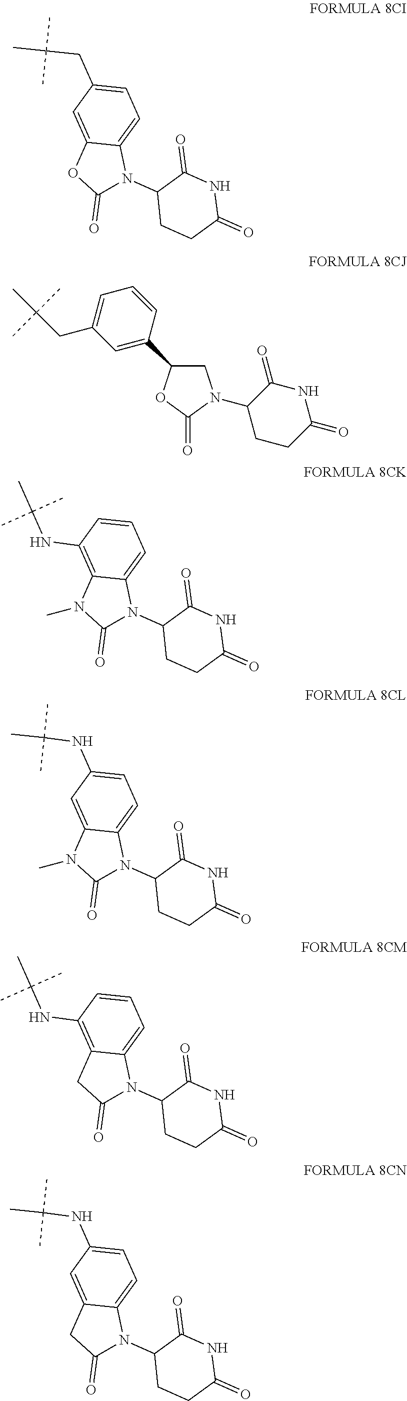

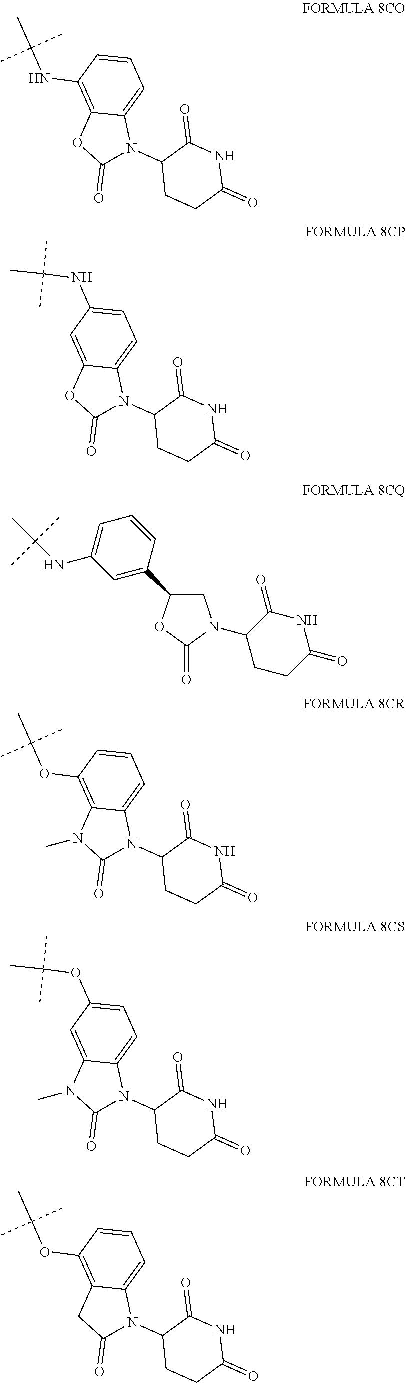

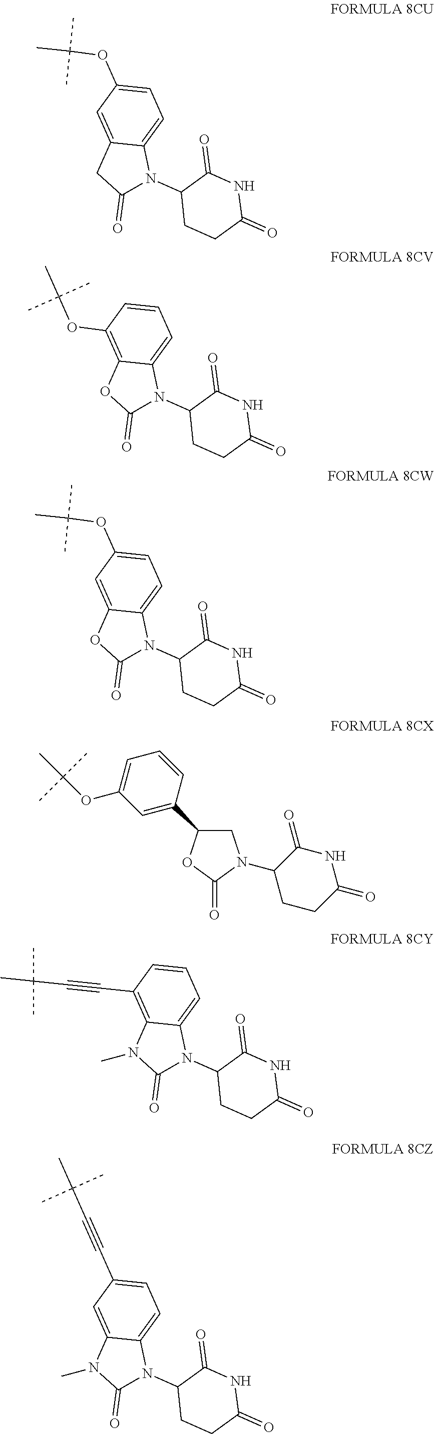

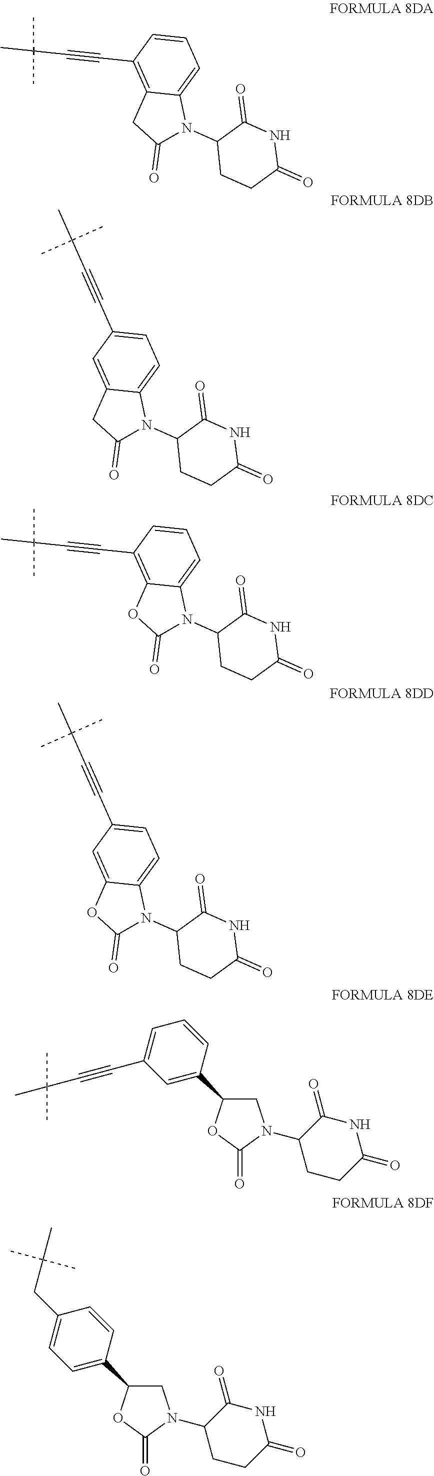

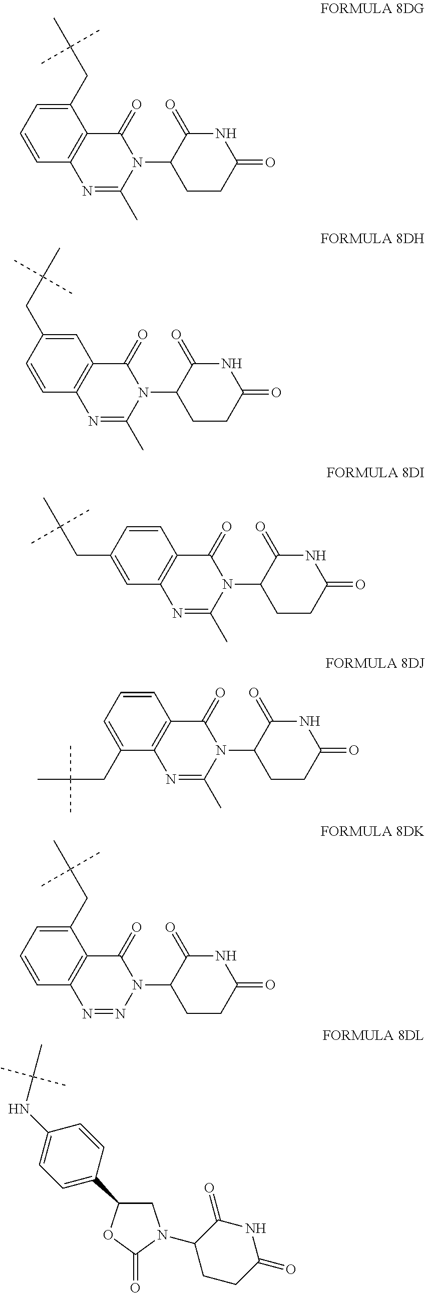

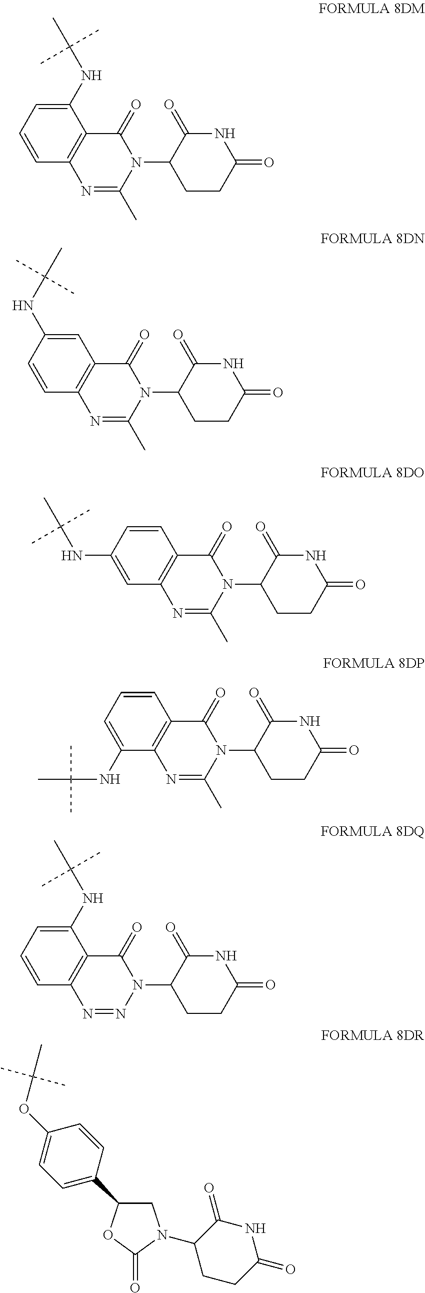

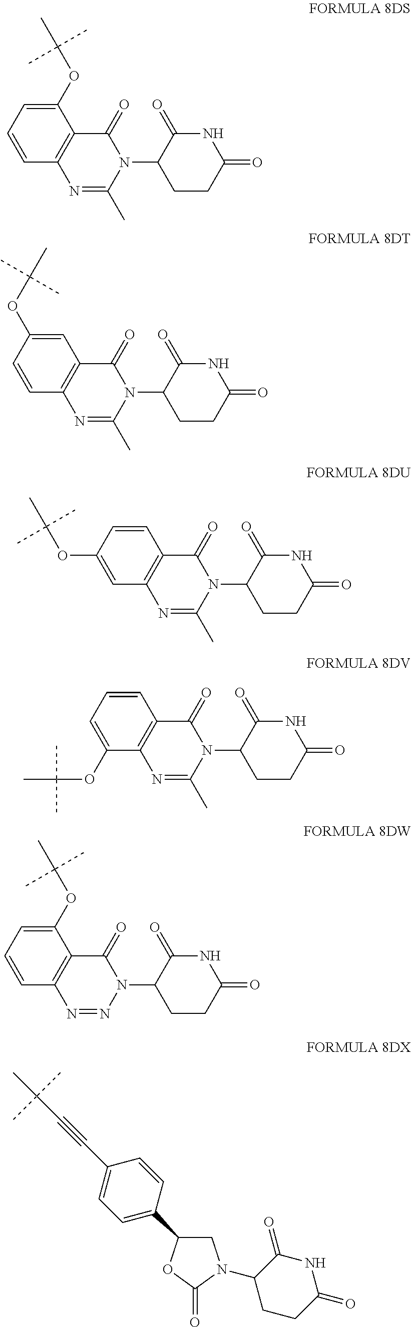

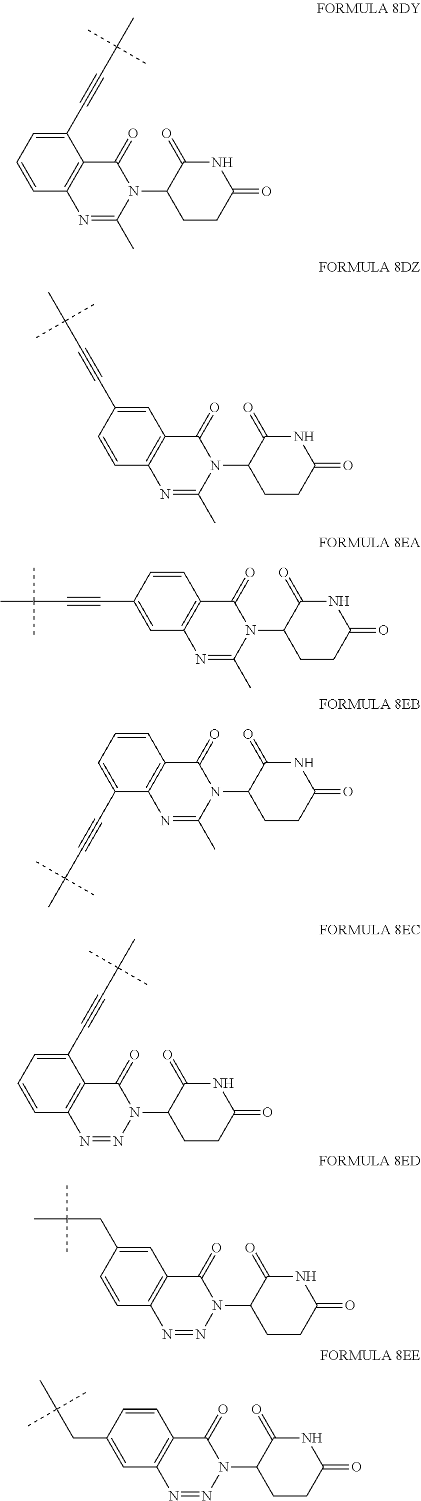

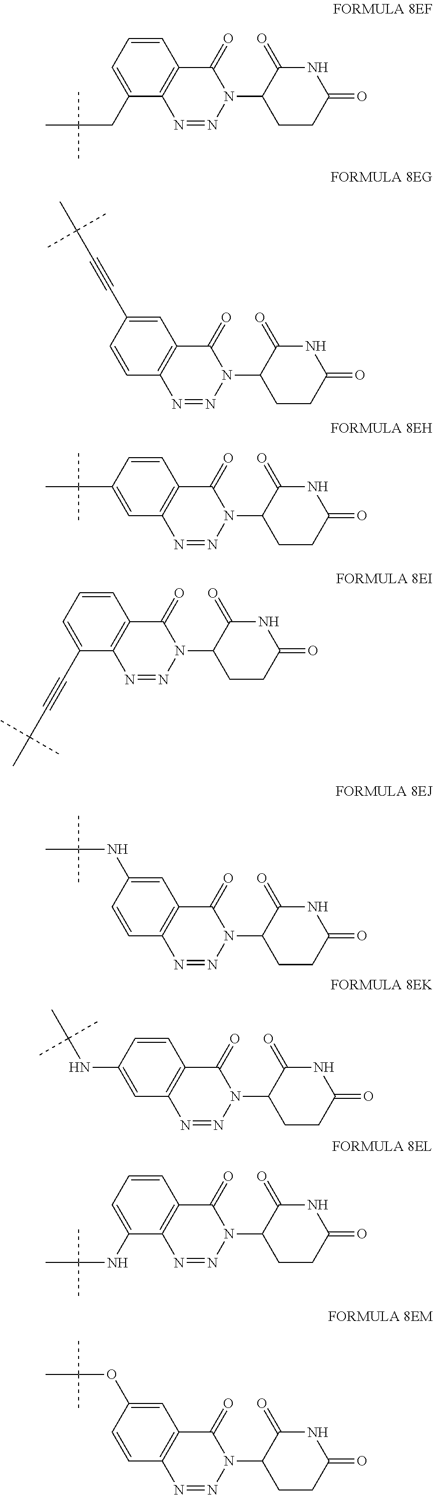

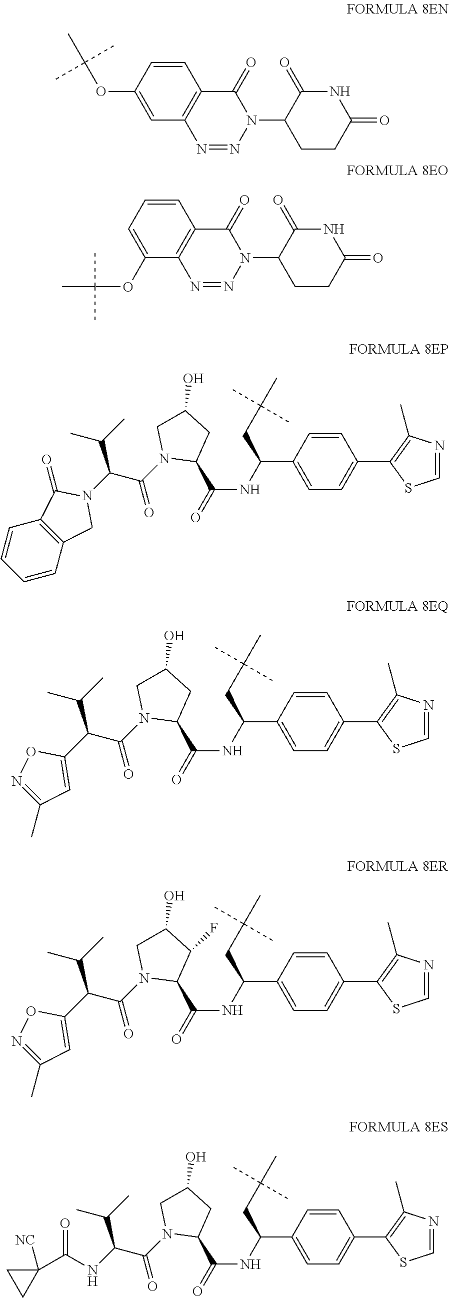

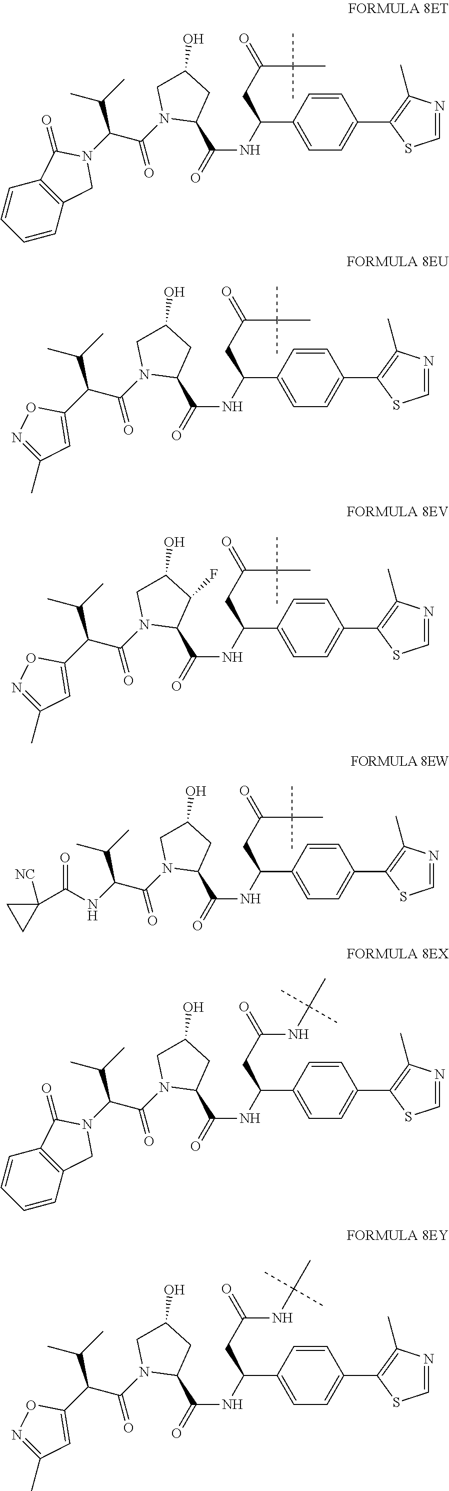

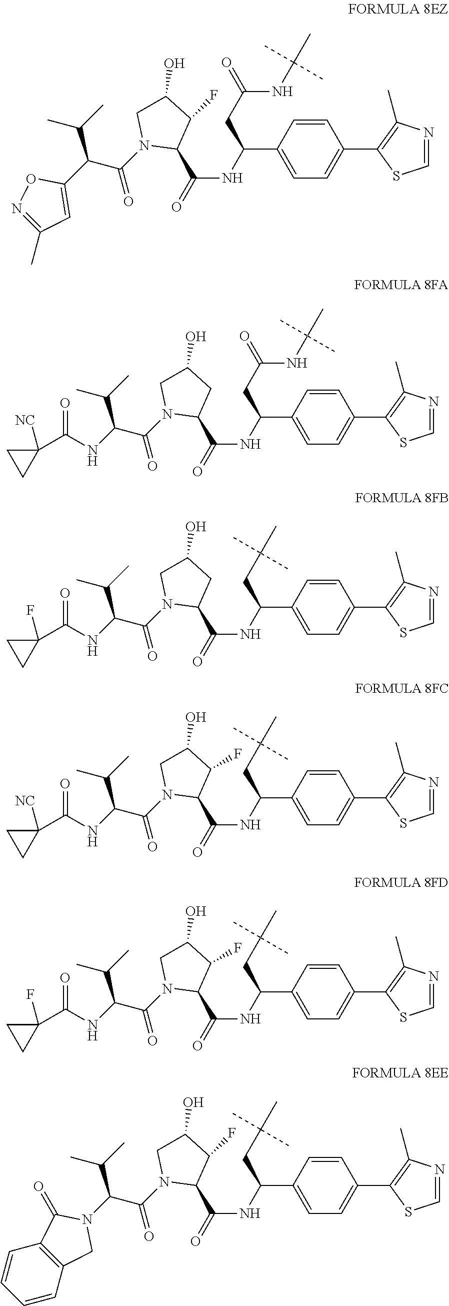

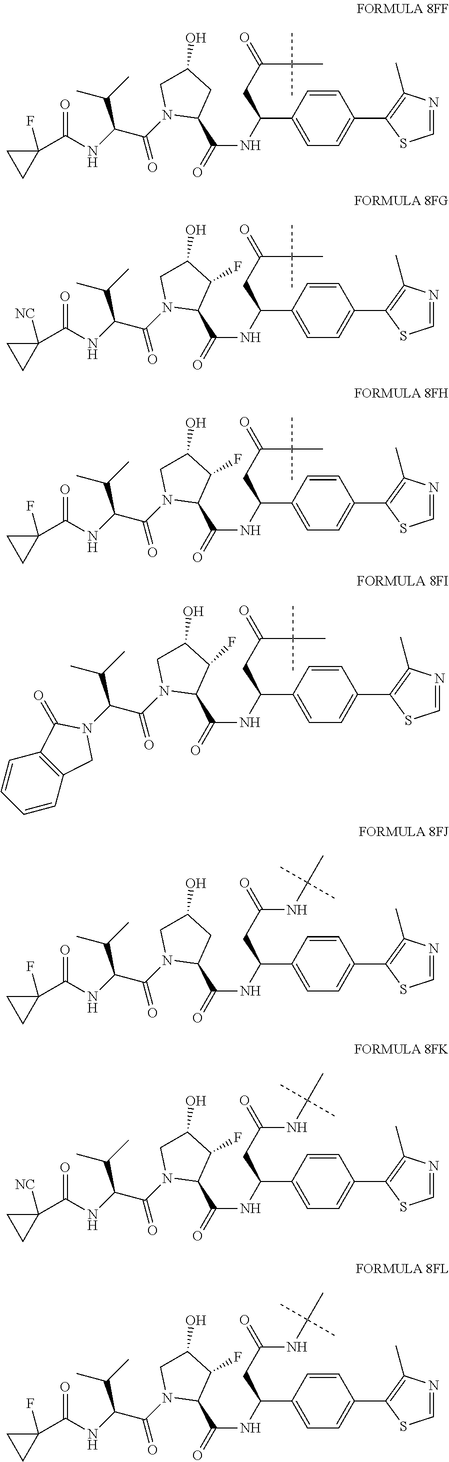

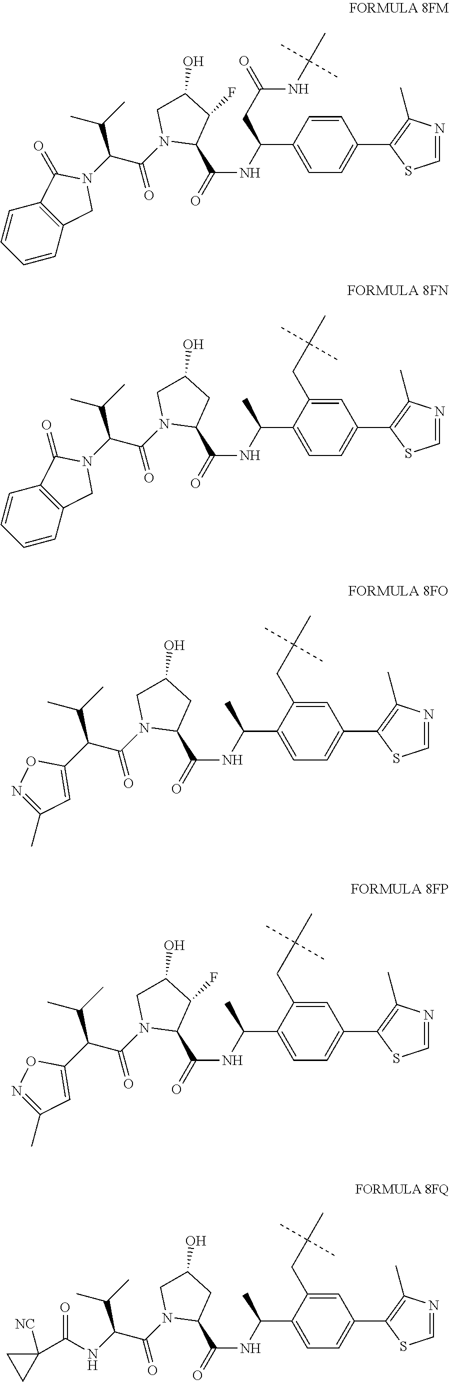

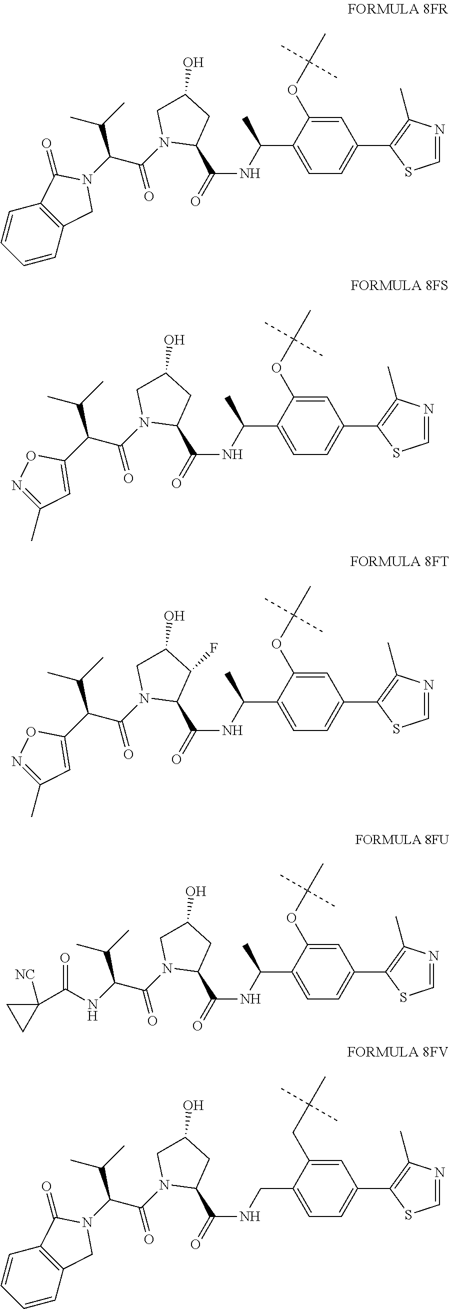

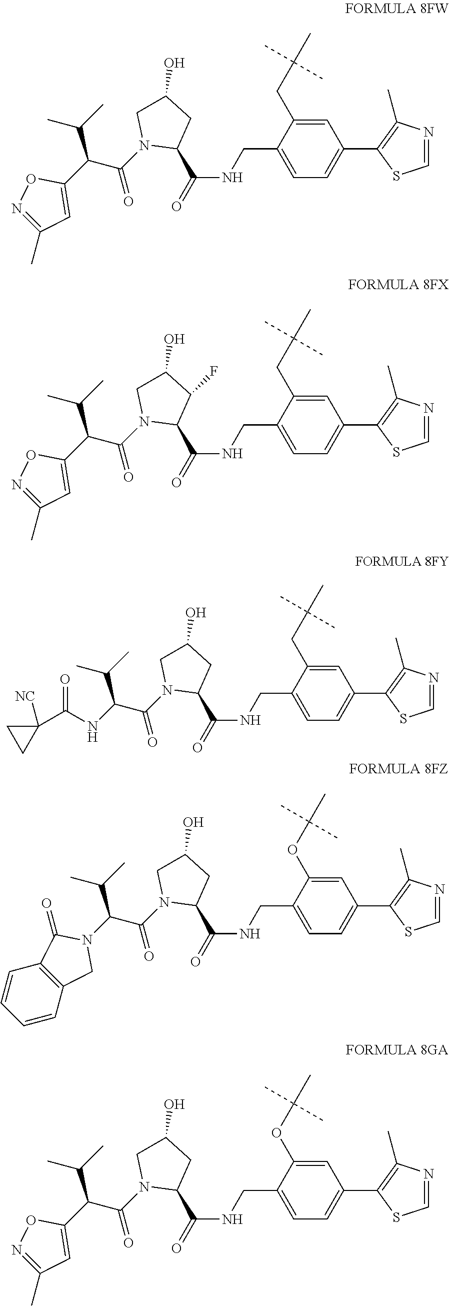

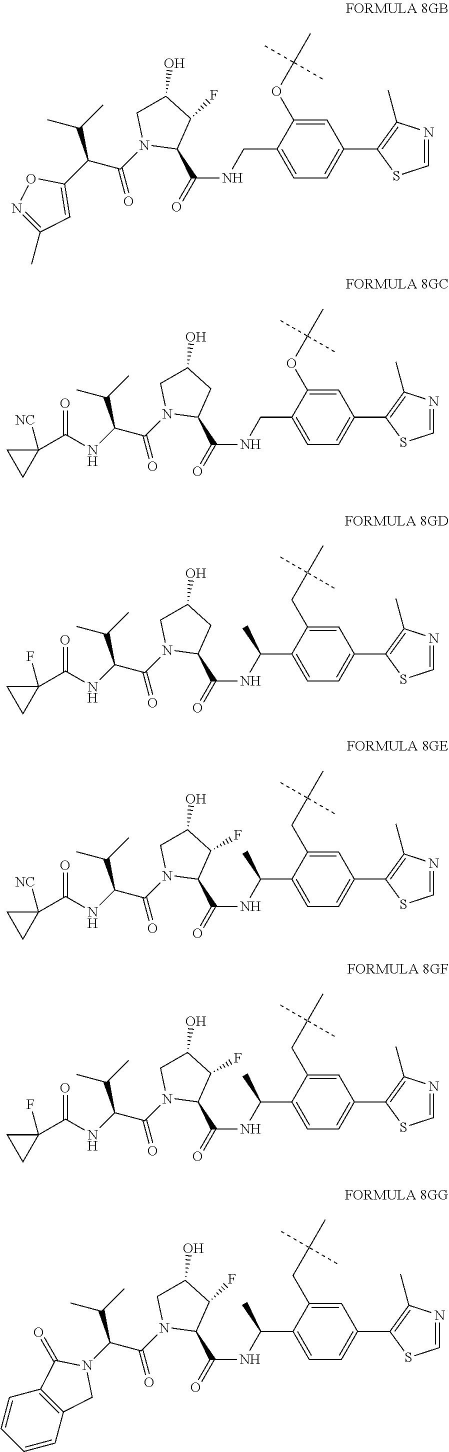

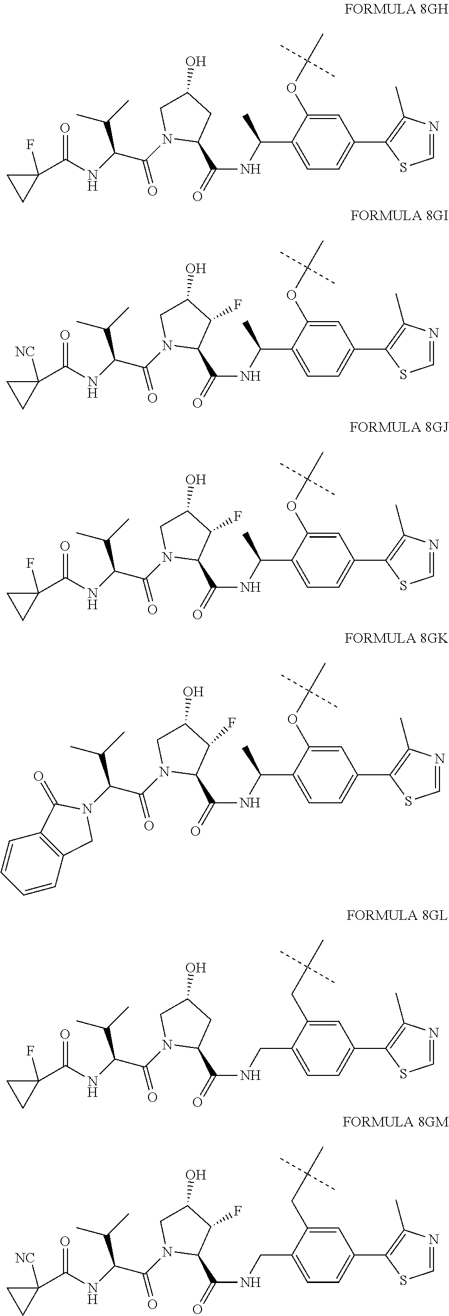

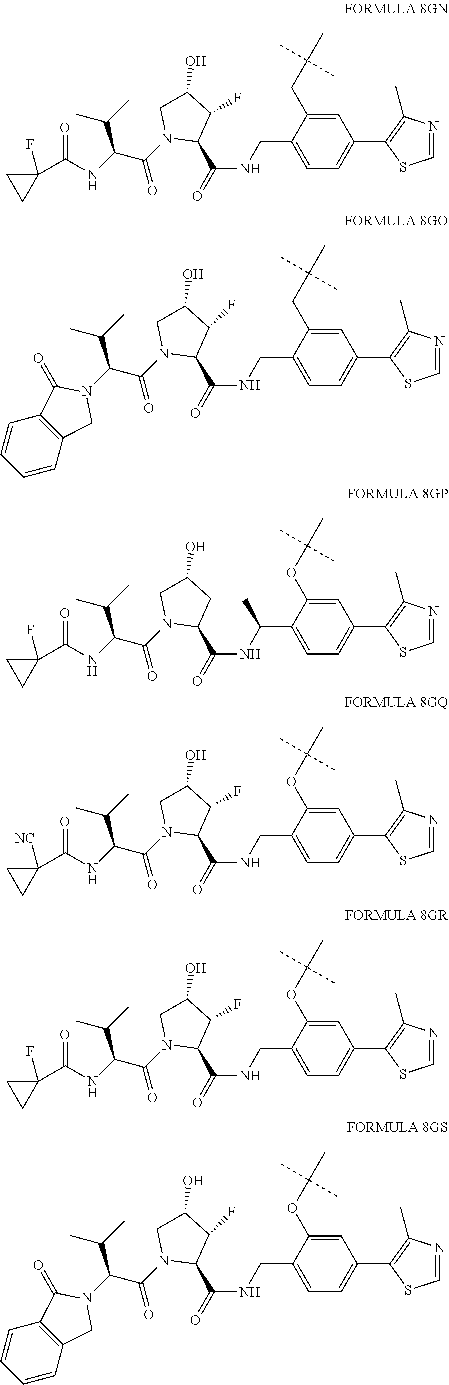

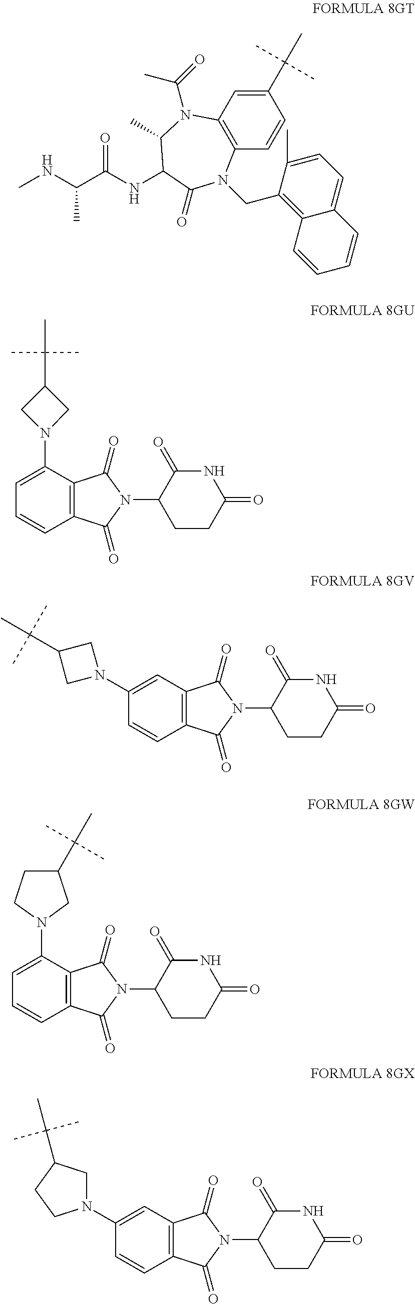

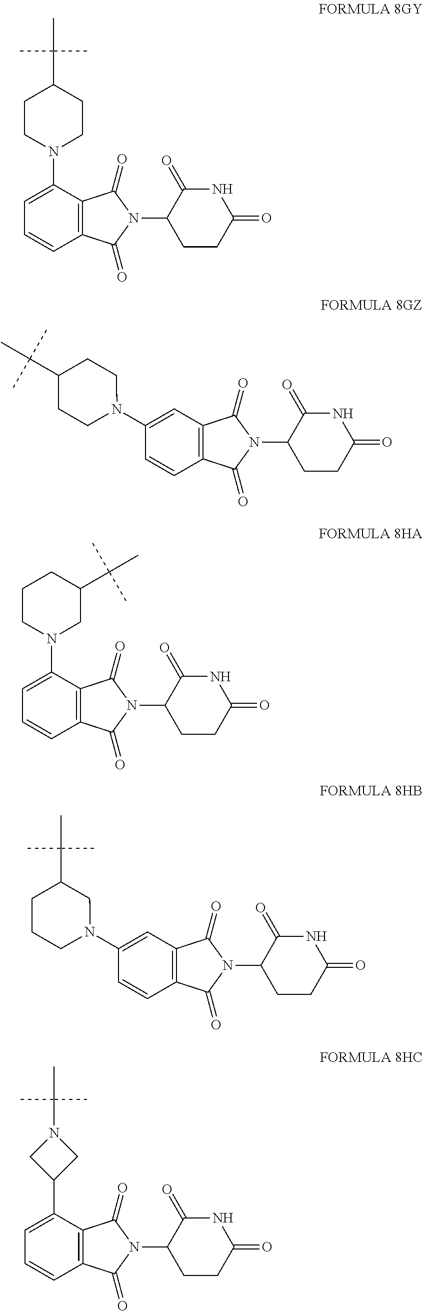

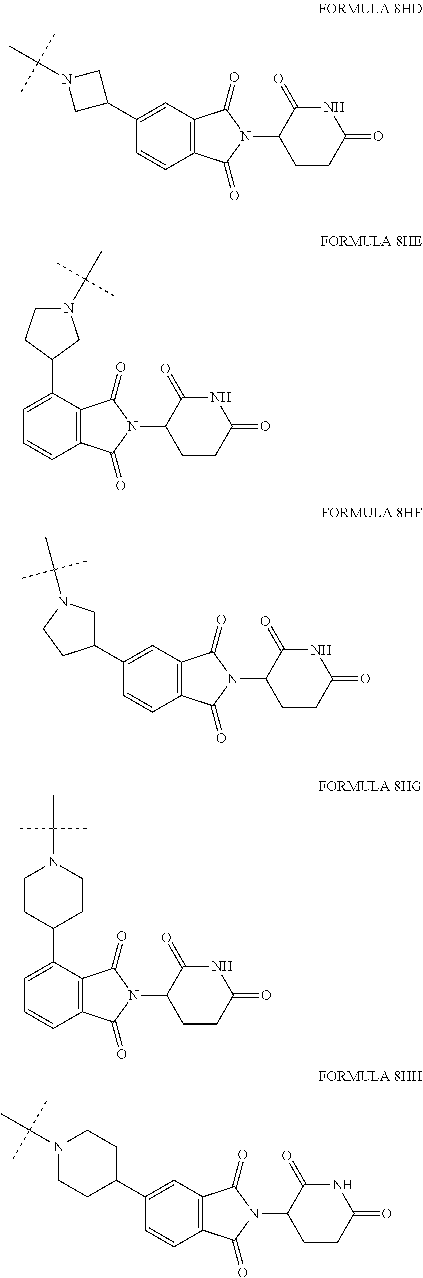

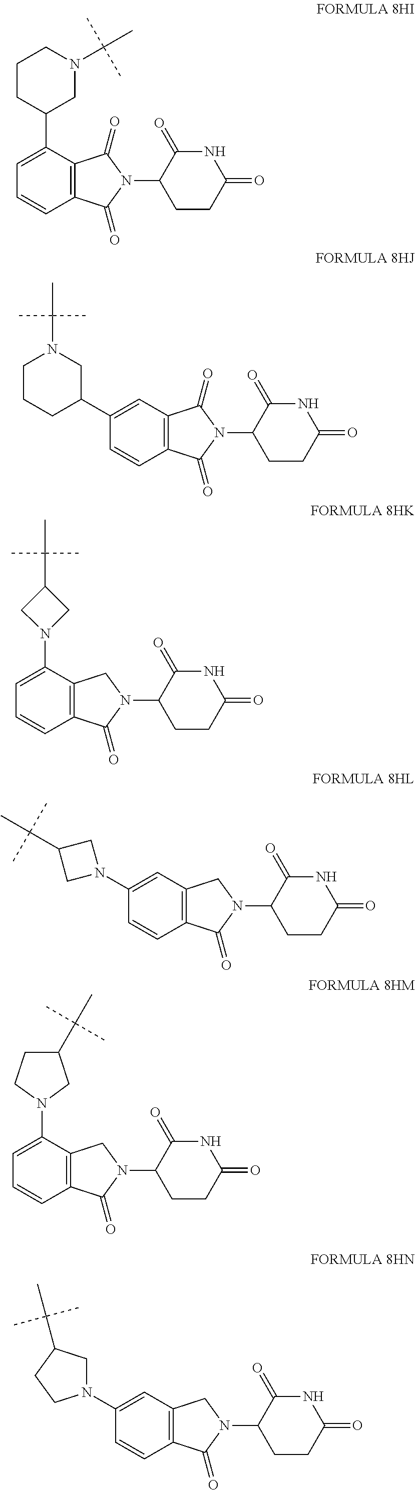

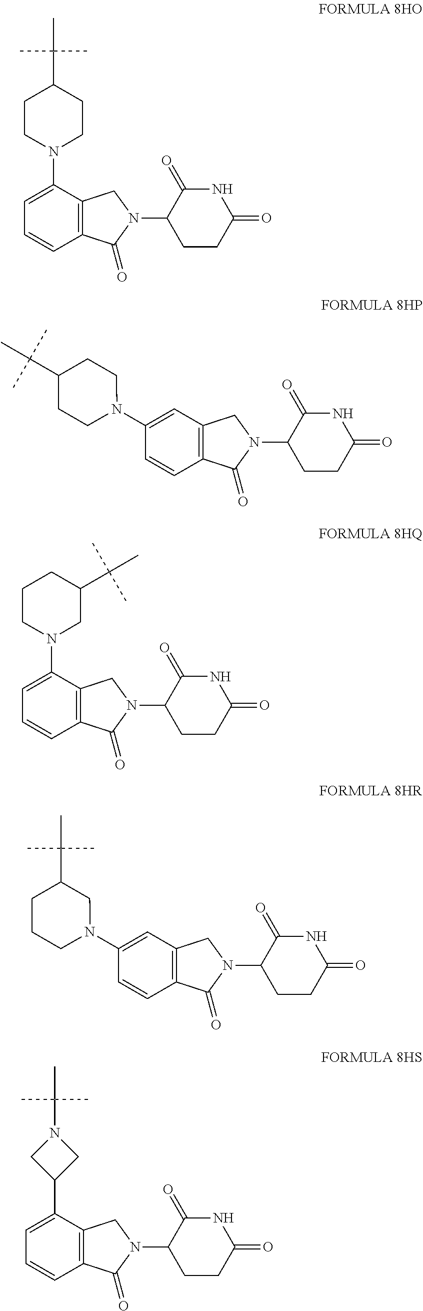

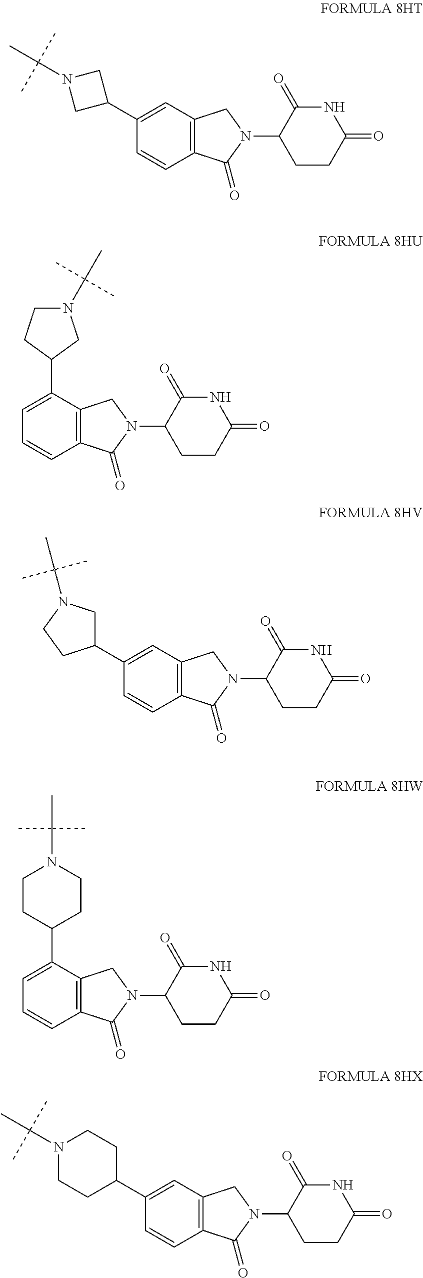

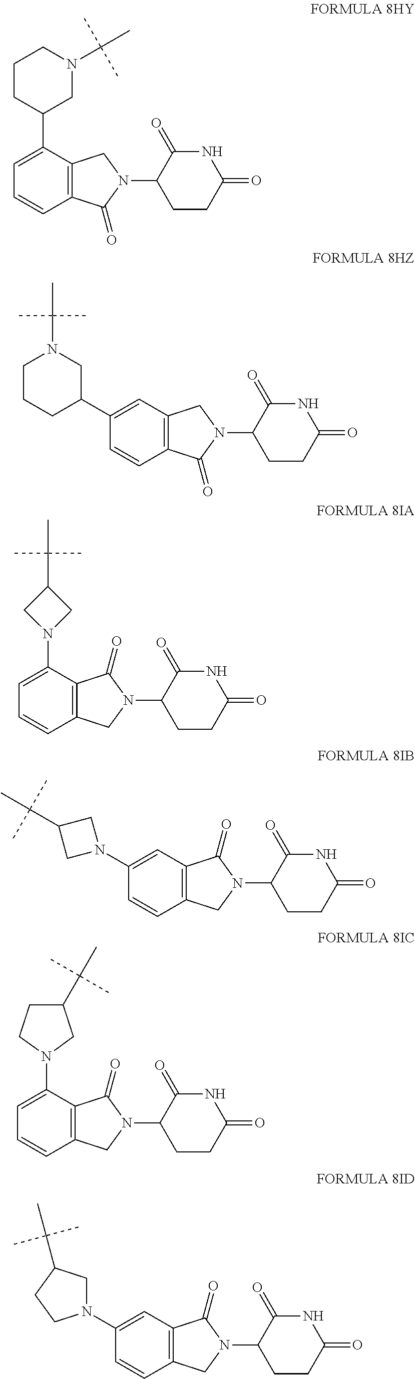

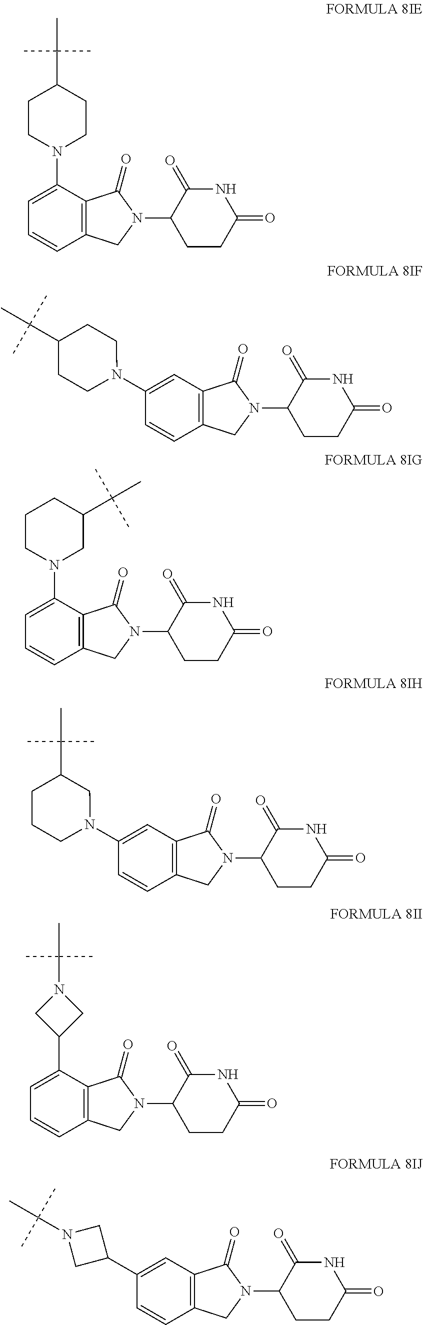

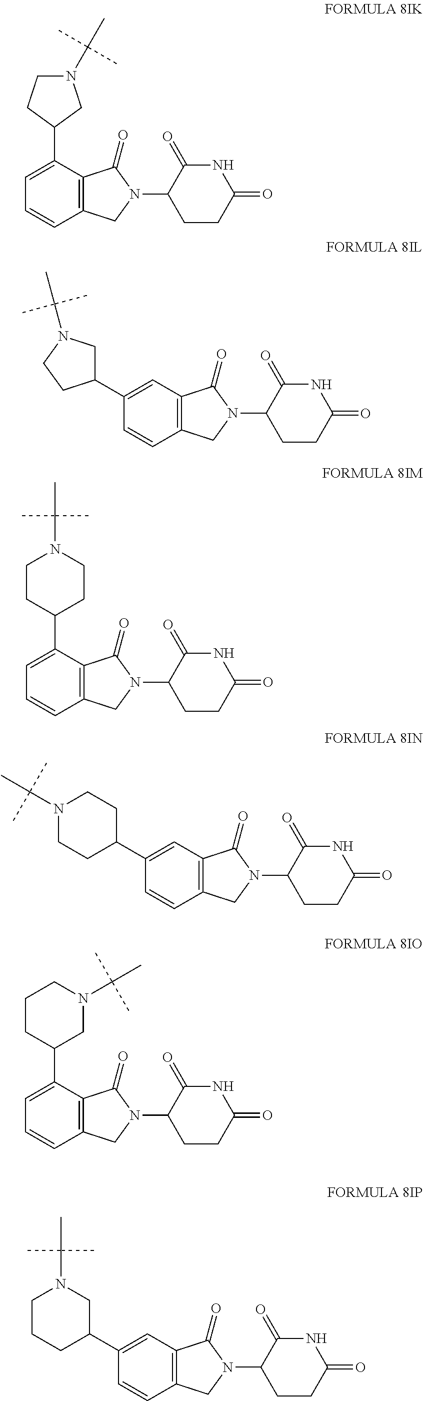

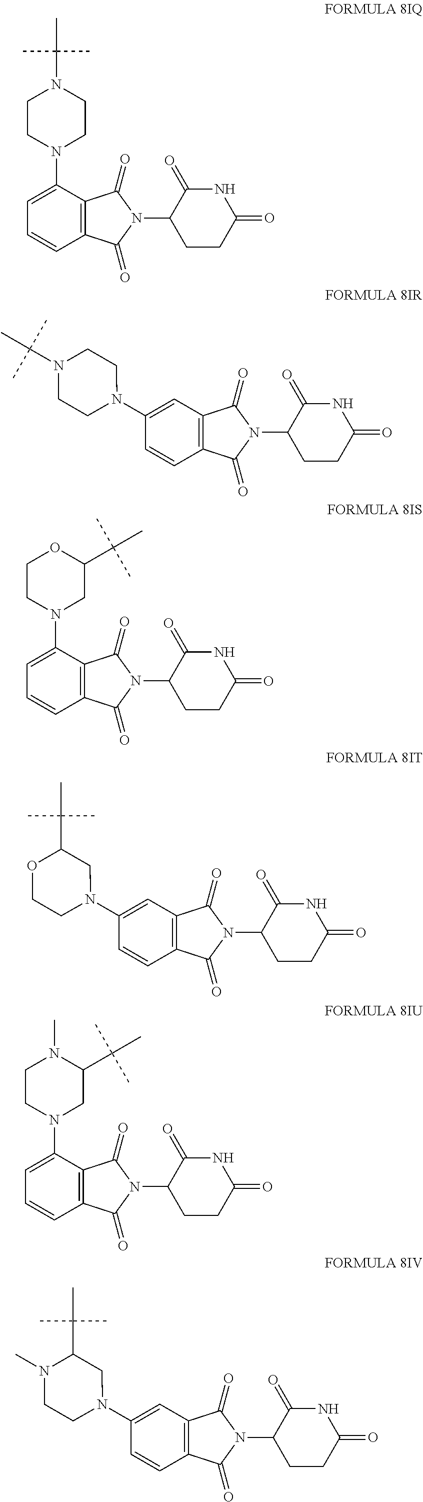

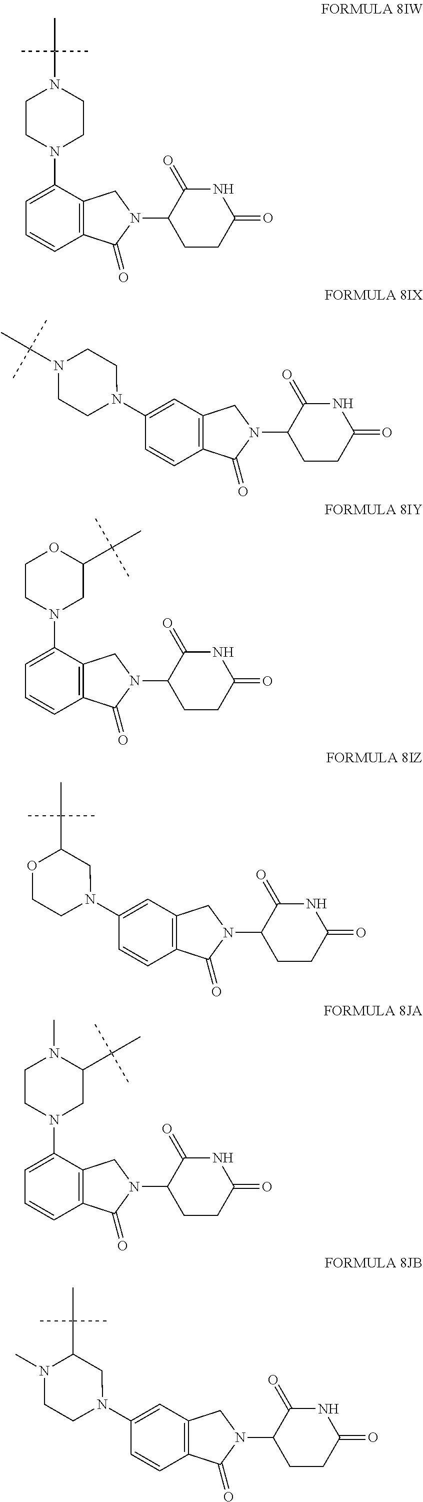

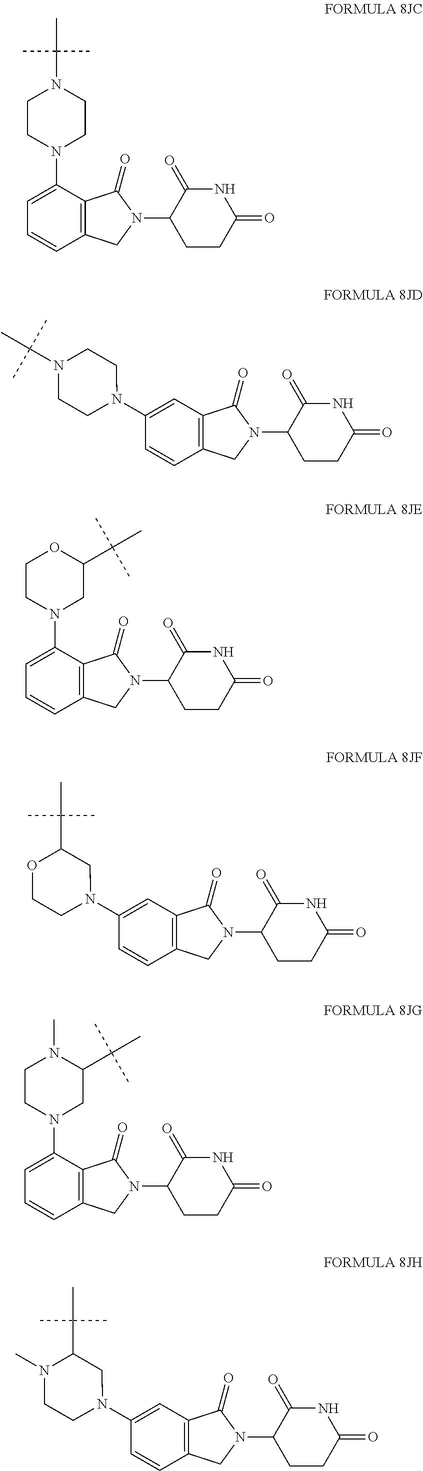

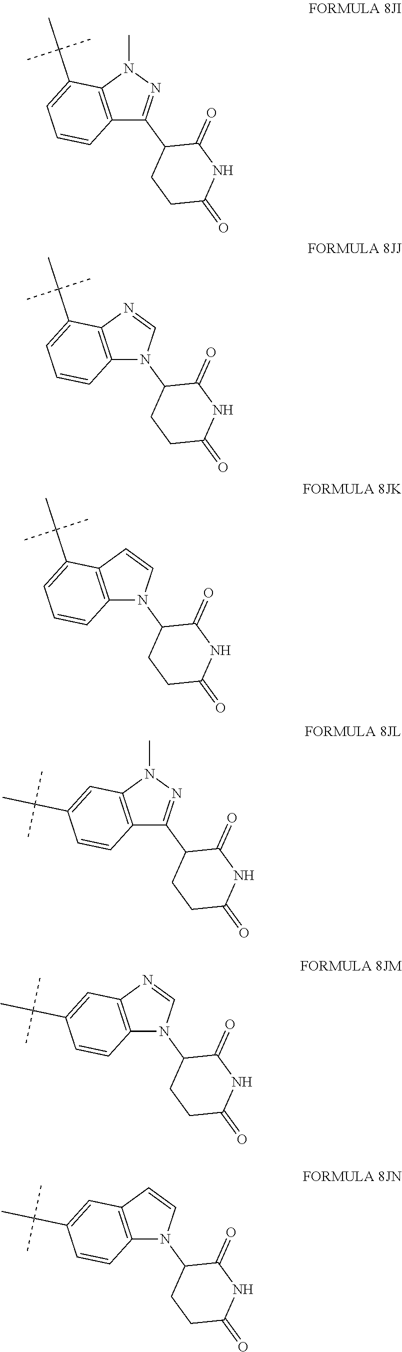

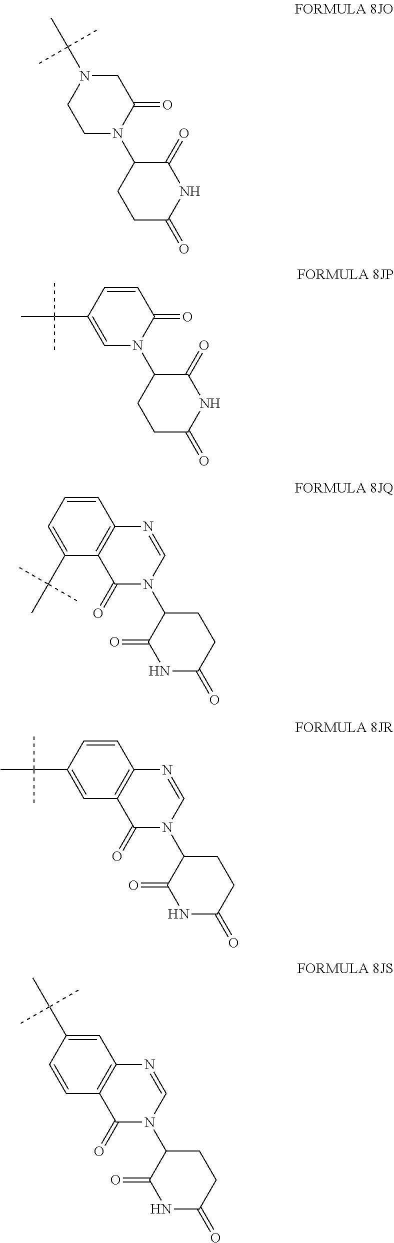

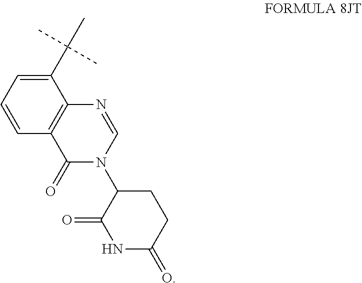

66. The heterobifunctional compound of claim 1, wherein
(III-1) the linker moiety is of FORMULA 9: 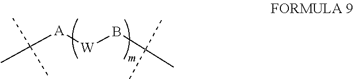
wherein
A, W, and B, at each occurrence, are independently selected from null, or bivalent moiety selected from R′—R″, R′COR″, R′CO₂R″, R′C(O)N(R¹)R″, R′C(S)N(R¹)R″, R′OR″, R′OC(O)R″, R′OC(O)OR″, R′OCON(R¹)R″, R′SR″, R′SOR″, R′SO₂R″, R′SO₂N(R′)R″, R′N(R¹)R″, R′N(R¹)COR″, R′N(R¹)C(O)OR″, R′N(R¹)CON(R²)R″, R′N(R¹)C(S)R″, R′N(R¹)S(O)R″, R′N(R¹)S(O)₂R″, and R′N(R¹)S(O)₂N(R²)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ Spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkyl), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R¹ and R² are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or
R′ and R″, R¹ and R², R′ and R¹, R′ and R², R″ and R¹, R″ and R² together with the atom to which they are connected form a 3-20 membered cycloalkyl or 4-20 membered heterocyclyl ring; and
m is 0 to 15; or
(III-2) the linker moiety is of FORMULA 9A: 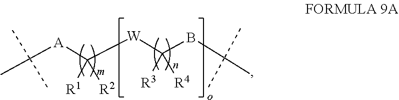
wherein
R¹, R², R³ and R⁴, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl, or
R¹ and R², R³ and R⁴ together with the atom to which they are connected form a 3-20 membered cycloalkyl or 4-20 membered heterocyclyl ring;
A, W, and B, at each occurrence, are independently selected from null, or bivalent moiety selected from R′—R″, R′COR″, R′CO₂R″, R′C(O)N(R⁵)R″, R′C(S)N(R⁵)R″, R′OR″, R′OC(O)R″, R′OC(O)OR″, R′OCON(R⁵)R″, R′SR″, R′SOR″, R′SO₂R″, R′SO₂N(R⁵)R″, R′N(R⁵)R″, R′N(R⁵)COR″, R′N(R⁵)C(O)OR″, R′N(R⁵)CON(R⁶)R″, R′N(R⁵)C(S)R″, R′N(R⁵)S(O)R″, R′N(R⁵)S(O)₂R″, and R′N(R⁵)S(O)₂N(R⁶)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁵ and R⁶ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or
R′ and R″, R⁵ and R⁶, R′ and R⁵, R′ and R⁶, R″ and R⁵, R″ and R⁶ together with the atom to which they are connected form a 3-20 membered cycloalkyl or 4-20 membered heterocyclyl ring;
m is 0 to 15;
n, at each occurrence, is 0 to 15; and
o is 0 to 15; or
(III-3) the linker moiety is of FORMULA 9B: 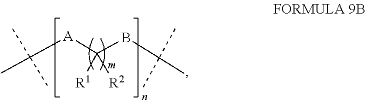
wherein
R¹ and R², at each occurrence, are independently selected from hydrogen, halogen, hydroxyl, amino, cyano, nitro, and optionally substituted C₁-C₈ alkyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, C₁-C₈alkylaminoC₁-C₈alkyl, optionally substituted 3-10 membered carbocyclyl, optionally substituted 3-8 membered cycloalkoxy, optionally substituted 3-10 membered carbocyclylamino, optionally substituted 4-10 membered heterocyclyl, optionally substituted aryl, and optionally substituted heteroaryl, or
R¹ and R² together with the atom to which they are connected form a 3-20 membered cycloalkyl or 4-20 membered heterocyclyl ring;
A and B, at each occurrence, are independently selected from null, or bivalent moiety selected from R′—R″, R′COR″, R′CO₂R″, R′C(O)N(R³)R″, R′C(S)N(R³)R″, R′OR″, R′OC(O)R″, R′OC(O)OR″, R′OCONR³R″, R′SR″, R′SOR″, R′SO₂R″, R′SO₂N(R³)R″, R′N(R³)R″, R′N(R³)COR″, R′N(R³)C(O)OR″, R′N(R³)CON(R⁴)R″, R′N(R³)C(S)R″, R′N(R³)S(O)R″, R′N(R³)S(O)₂R″, and R′N(R³)S(O)₂N(R⁴)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r}(preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R³ and R⁴ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or
R′ and R″, R³ and R⁴, R′ and R³, R′ and R⁴, R″ and R³, R″ and R⁴ together with the atom to which they are connected form a 3-20 membered cycloalkyl or 4-20 membered heterocyclyl ring;
each m is 0 to 15; and
n is 0 to 15; or
(III-4) the linker moiety is of FORMULA 9C: 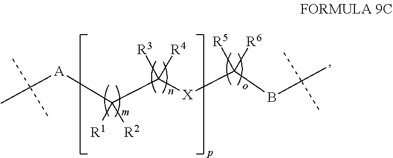
wherein
X is selected from O, NH, and NR⁷;
R¹, R², R³, R⁴, R⁵, and R⁶, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxy C₁-C₈ alkyl, optionally substituted C₁-C₈ haloalkyl, optionally substituted C₁-C₈ hydroxyalkyl, optionally substituted C₁-C₈ alkylamino, optionally substituted C₁-C₈ alkylaminoC₁-C₈ alkyl, optionally substituted 3-10 membered carbocyclyl, optionally substituted 3-8 membered cycloalkoxy, optionally substituted 4-10 membered heterocyclyl, optionally substituted aryl, and optionally substituted heteroaryl;
A and B are independently selected from null, or bivalent moiety selected from R′—R″, R′COR″, R′CO₂R″, R′C(O)N(R⁸)R″, R′C(S)N(R⁸)R″, R′OR″, R′OC(O)R″, R′OC(O)OR″, R′OCON(R⁸)R″, R′SR″, R′SOR″, R′SO₂R″, R′SO₂N(R⁸)R″, R′N(R⁸)R″, R′N(R⁸)COR″, R′N(R⁸)C(O)OR″, R′N(R⁸)CON(R⁹)R″, R′N(R⁸)C(S)R″, R′N(R⁸)S(O)R″, R′N(R⁸)S(O)₂R″, and R′N(R⁸)S(O)₂N(R⁹)R″, optionally substituted C₁-C₈ alkylene, optionally substituted C₂-C₈ alkenylene, optionally substituted C₂-C₈ alkynylene, optionally substituted C₁-C₈alkoxyC₁-C₈alkylene, optionally substituted C₁-C₈ haloalkylene, optionally substituted C₁-C₈ hydroxyalkylene, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted 3-10 membered carbocyclyl, optionally substituted 4-10 membered heterocyclyl, optionally substituted aryl, and optionally substituted heteroaryl, wherein
R′ and R″ are independently selected from null, optionally substituted (C₁-C₈ alkylene)-R^{r} (preferably, CH₂—R^{r}), optionally substituted R^{r}—(C₁-C₈ alkylene), optionally substituted (C₁-C₈ alkylene)-R^{r}—(C₁-C₈ alkylene), or a moiety comprising of optionally substituted C₁-C₈ alkyl, optionally substituted C₂-C₈ alkenyl, optionally substituted C₂-C₈ alkynyl, optionally substituted C₁-C₈ hydroxyalkyl, optionally substituted C₁-C₈alkoxyC₁-C₈alkyl, optionally substituted C₁-C₈alkylaminoC₁-C₈alkyl, optionally substituted C₁-C₈ haloalkyl, optionally substituted C₁-C₈ alkylene, optionally substituted C₂-C₈ alkenylene, optionally substituted C₂-C₈ alkynylene, optionally substituted C₁-C₈ hydroxyalkylene, optionally substituted C₁-C₈alkoxyC₁-C₈alkylene, optionally substituted C₁-C₈alkylaminoC₁-C₈alkylene, optionally substituted C₁-C₈ haloalkylene, optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R^{r} is selected from optionally substituted 3-10 membered carbocyclyl, optionally substituted 4-10 membered heterocyclyl, optionally substituted C₃-C₁₃ fused cycloalkyl, optionally substituted C₃-C₁₃ fused heterocyclyl, optionally substituted C₃-C₁₃ bridged cycloalkyl, optionally substituted C₃-C₁₃ bridged heterocyclyl, optionally substituted C₃-C₁₃ spiro cycloalkyl, optionally substituted C₃-C₁₃ spiro heterocyclyl, optionally substituted aryl, and optionally substituted heteroaryl;
R⁷, R⁸ and R⁹ are independently selected from hydrogen, optionally substituted C₁-C₈ alkyl, optionally substituted C₂-C₈ alkenyl, optionally substituted C₂-C₈ alkynyl, optionally substituted C₁-C₈ alkoxyalkyl, optionally substituted C₁-C₈ haloalkyl, optionally substituted C₁-C₈ hydroxyalkyl, optionally substituted C₁-C₈alkylaminoC₁-C₈alkyl, optionally substituted 3-10 membered carbocyclyl, optionally substituted 4-10 membered heterocyclyl, optionally substituted aryl, and optionally substituted heteroaryl; or
R′ and R″, R⁸ and R⁹, R′ and R⁸, R′ and R⁹, R″ and R⁸, R″ and R⁹ together with the atom to which they are connected form a 3-20 membered cycloalkyl or 4-20 membered heterocyclyl ring;
m, at each occurrence, is 0 to 15;
n, at each occurrence, is 0 to 15;
o is 0 to 15; and
p is 0 to 15.

67-69. (canceled)

70. The heterobifunctional compound of claim 66, wherein A and B, at each occurrence, are independently selected from null, CO, NH, NH—CO, CO—NH, CH₂—NH—CO, CH₂—CO—NH, NH—CO—CH₂, CO—NH—CH₂, CH₂—NH—CH₂—CO—NH, CH₂—NH—CH₂—NH—CO, —CO—NH, CO—NH— CH₂—NH—CH₂, CH₂—NH—CH₂.

71. (canceled)

72. The heterobifunctional compound of claim 66, wherein the linker moiety comprises one or more rings selected from the group consisting of FORMULAE C1a, C2a, C3a, C4a and C5a 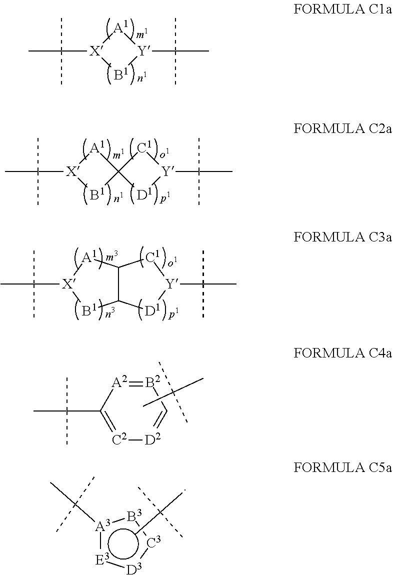
wherein
X′ and Y′ are independently selected from N, CR^{b};
A¹, B¹, C¹ and D¹, at each occurrence, are independently selected from null, O, CO, SO, SO₂, NR^{b}, CR^{b}R^{c};
A², B², C², and D², at each occurrence, are independently selected from N, CR^{b};
A³, B³, C³, D³, and E³, at each occurrence, are independently selected from N, O, S, NR^{b}, CR^{b};
R^{b} and R^{c}, at each occurrence, are independently selected from hydrogen, halogen, hydroxyl, amino, cyano, nitro, optionally substituted C₁-C₈ alkyl, optionally substituted C₂-C₈ alkenyl, optionally substituted C₂-C₈ alkynyl, optionally substituted C₁-C₈ alkoxy, optionally substituted C₁-C₈ alkoxyalkyl, optionally substituted C₁-C₈ haloalkyl, optionally substituted C₁-C₈ hydroxyalkyl, optionally substituted C₁-C₈ alkylamino, and optionally substituted C₁-C₈ alkylaminoC₁-C₈ alkyl, optionally substituted 3-10 membered carbocyclyl, optionally substituted 3-8 membered cycloalkoxy, optionally substituted 3-10 membered carbocyclylamino, optionally substituted 4-8 membered heterocyclyl, optionally substituted aryl, and optionally substituted heteroaryl; and
m¹, n¹, o¹ and p¹ are independently selected from 0, 1, 2, 3, 4 and 5.

73. (canceled)

74. The heterobifunctional compound of claim 1, wherein the length of the linker is the length of the linker is 2 to 12 chain atoms.

75. The heterobifunctional compound of claim 1, wherein the linker is selected from —(CO)—(CH₂)₁₋₈—, —(CH₂)₁₋₉—, —(CH₂)₁₋₂—(CO)—NH—(CH₂)₂₋₉—, —(CH₂)₁₋₂—(CO)—NH—(CH₂)₁₋₃—(OCH₂CH₂)₁₋₇, and —(CH₂)₀₋₁—(CO)—(CH₂)₁₋₃—(OCH₂CH₂)₁₋₇—.

76. The heterobifunctional compound of claim 1, wherein the linker is —(CO)—(CH₂)₁₋₈—, —(CH₂)₁₋₉—, —(CH₂)₁₋₂(CO)—NH—(CH₂)₂₋₉—, or —(CH₂)₁₋₂—(CO)—NH—(CH₂)₁₋₃—(OCH₂CH₂)₁₋₇—.

77. The heterobifunctional compound of claim 1, wherein the heterobifunctional compound is selected from the group consisting of JA-001, JA-002, JA-003, JA-004, JA-005, JA-010, JA-011, JA-012, JA-013, JA-014, JA-038, JA-039, JA-040, JA-041, JA-042, JA-043, JA-044, JA-045, JA-046, JA-047, JA-048, JA-049, JA-050, JA-051, JA-052, JA-053, JA-054, JA-055, JA-056, JA-057, JA-058, JA-059, JA-060, JA-061, JA-062, JA-063, JA-064, JA-065, JA-066, JA-067, JA-068, JA-069, JA-070, JA-071, JA-072, JA-073, JA-074, JA-075, JA-076, JA-077, JA-078, JA-079, JA-080, JA-081, JA-082, JA-083, JA-084, JA-085, JA-109, JA-110, JA-111, JA-112, JA-113, JA-114, JA-115, JA-116, JA-117, JA-118, JA-119, JA-120, JA-121, JA-122, JA-123, JA-124, JA-125, JA-126, JA-127, JA-128, JA-129, JA-130, JA-131, JA-132, JA-133, JA-134, JA-135, JA-136, JA-137, JA-138, JA-139, JA-140, JA-141, JA-142, JA-143, JA-144, JA-145, JA-146, JA-147, JA-148, JA-149, JA-150, JA-151, JA-152, JA-153, JA-154, JA-155, JA-156, JA-157, JA-158, JA-159, JA-160, JA-161, JA-162, JA-163, JA-164, JA-165, JA-166, JA-167, JA-168, JA-169, JA-170, JA-171, JA-172, JA-173, JA-174, JA-175, JA-176, JA-177, JA-178, JA-179, JA-180, JA-181, JA-182, JA-183, JA-184, JA-185, JA-186, JA-187, JA-188, JA-189, JA-190, JA-191, JA-192, JA-193, JA-194, JA-195, JA-196, JA-197, JA-198, JA-199, JA-200, JA-201, JA-202, JA-203, JA-204, JA-205, JA-206, JA-207, JA-208, JA-209, JA-210, JA-211, JA-212, JA-213, JA-214, JA-215, JA-216, JA-248, JA-249, JA-250, JA-251, JA-252, JA-253, JA-254, JA-255, JA-256, JA-257, JA-258, JA-259, JA-260, JA-261, JA-262, JA-263, JA-264, JA-265, JA-266, JA-267, JA-268, JA-269, JA-270, JA-271, JA-272, JA-273, JA-274, JA-275, JA-278, JA-279, JA-280, JA-281, JA-282, JA-283, JA-284, JA-285, JA-286, JA-287, JA-288, JA-289, JA-290, JA-291, JA-292, and JA-293 or a pharmaceutically acceptable salt or analog thereof.

78. The heterobifunctional compound of claim 1, wherein the heterobifunctional compound is selected from the group consisting of
2-(4-(4-((4-((3-(N-(tert-butyl)sulfamoyl)phenyl)amino)-5-methylpyrimidin-2-yl)amino)phenyl)piperazin-1-yl)-N-(2-(2-((2-(2,6-dioxopiperidin-3-yl)-1,3-dioxoisoindolin-4-yl)amino)ethoxy)ethyl)acetamide (JA-087);
2-(4-(4-((4-((3-(N-(tert-butyl)sulfamoyl)phenyl)amino)-5-methylpyrimidin-2-yl)amino)phenyl)piperazin-1-yl)-N-(5-((2-(2,6-dioxopiperidin-3-yl)-1,3-dioxoisoindolin-4-yl)amino)pentyl)acetamide (JA-093);
2-(4-(4-((4-((3-(N-(tert-butyl)sulfamoyl)phenyl)amino)-5-methylpyrimidin-2-yl)amino)phenyl)piperazin-1-yl)-N-(6-((2-(2,6-dioxopiperidin-3-yl)-1,3-dioxoisoindolin-4-yl)amino)hexyl)acetamide (JA-094);
2-(4-(4-((4-((3-(N-(tert-butyl)sulfamoyl)phenyl)amino)-5-methylpyrimidin-2-yl)amino)phenyl)piperazin-1-yl)-N-(7-((2-(2,6-dioxopiperidin-3-yl)-1,3-dioxoisoindolin-4-yl)amino)heptyl)acetamide (JA-095);
2-(4-(4-((4-((3-(N-(tert-butyl)sulfamoyl)phenyl)amino)-5-methylpyrimidin-2-yl)amino)phenyl)piperazin-1-yl)-N-(8-((2-(2,6-dioxopiperidin-3-yl)-1,3-dioxoisoindolin-4-yl)amino)octyl)acetamide (JA-096);
2-(4-(4-((4-((3-(N-(tert-butyl)sulfamoyl)phenyl)amino)-5-methylpyrimidin-2-yl)amino)phenyl)piperazin-1-yl)-N-(17-((2-(2,6-dioxopiperidin-3-yl)-1,3-dioxoisoindolin-4-yl)amino)-3,6,9,12,15-pentaoxaheptadecyl)acetamide (JA-224);
N-(tert-butyl)-3-((2-((4-(4-((2-(2,6-dioxopiperidin-3-yl)-1,3-dioxoisoindolin-5-yl)glycyl)piperazin-1-yl)phenyl)amino)-5-methylpyrimidin-4-yl)amino)benzenesulfonamide (JA-225);
N-(tert-butyl)-3-((2-((4-(4-(8-((2-(2,6-dioxopiperidin-3-yl)-1,3-dioxoisoindolin-5-yl)amino)octanoyl)piperazin-1-yl)phenyl)amino)-5-methylpyrimidin-4-yl)amino)benzenesulfonamide (JA-231);
N-(tert-butyl)-3-((2-((4-(4-(3-(2-(2-((2-(2,6-dioxopiperidin-3-yl)-1,3-dioxoisoindolin-5-yl)amino)ethoxy)ethoxy)propanoyl)piperazin-1-yl)phenyl)amino)-5-methylpyrimidin-4-yl)amino)benzenesulfonamide (JA-233);
2-(4-(4-((4-((3-(N-(tert-butyl)sulfamoyl)phenyl)amino)-5-methylpyrimidin-2-yl)amino)phenyl)piperazin-1-yl)-N-(6-((2-(2,6-dioxopiperidin-3-yl)-1,3-dioxoisoindolin-5-yl)amino)hexyl)acetamide (JA-245); and
2-(4-(4-((4-((3-(N-(tert-butyl)sulfamoyl)phenyl)amino)-5-methylpyrimidin-2-yl)amino)phenyl)piperazin-1-yl)-N-(7-((2-(2,6-dioxopiperidin-3-yl)-1,3-dioxoisoindolin-5-yl)amino)heptyl)acetamide (JA-246);
and a pharmaceutically acceptable salt or analog thereof.

79. The heterobifunctional compound of claim 1, wherein the heterobifunctional compound is selected from the group consisting of
2-(4-(4-(8-(3,5-difluoro-4-(morpholinomethyl)phenyl)quinoxalin-2-yl)-1H-pyrazol-1-yl)piperidin-1-yl)-N-(7-((2-(2,6-dioxopiperidin-3-yl)-1,3-dioxoisoindolin-5-yl)amino)heptyl)acetamide (JA-179);
2-(4-(4-(8-(3,5-difluoro-4-(morpholinomethyl)phenyl)quinoxalin-2-yl)-1H-pyrazol-1-yl)piperidin-1-yl)-N-(8-((2-(2,6-dioxopiperidin-3-yl)-1,3-dioxoisoindolin-5-yl)amino)octyl)acetamide (JA-180);
5-((5-(4-(4-(8-(3,5-difluoro-4-(morpholinomethyl)phenyl)quinoxalin-2-yl)-1H-pyrazol-1-yl)piperidin-1-yl)-5-oxopentyl)amino)-2-(2,6-dioxopiperidin-3-yl)isoindoline-1,3-dione (JA-182);
5-((8-(4-(4-(8-(3,5-difluoro-4-(morpholinomethyl)phenyl)quinoxalin-2-yl)-1H-pyrazol-1-yl)piperidin-1-yl)-8-oxooctyl)amino)-2-(2,6-dioxopiperidin-3-yl)isoindoline-1,3-dione (JA-187);
5-((7-(4-(4-(8-(3,5-difluoro-4-(morpholinomethyl)phenyl)quinoxalin-2-yl)-1H-pyrazol-1-yl)piperidin-1-yl)-7-oxoheptyl)amino)-2-(2,6-dioxopiperidin-3-yl)isoindoline-1,3-dione (JA-188);
5-((6-(4-(4-(8-(3,5-difluoro-4-(morpholinomethyl)phenyl)quinoxalin-2-yl)-1H-pyrazol-1-yl)piperidin-1-yl)-6-oxohexyl)amino)-2-(2,6-dioxopiperidin-3-yl)isoindoline-1,3-dione (JA-189);
2-(2,6-Dioxopiperidin-3-yl)-5-((5-(4-(4-((5-(4-(methylsulfonyl)phenyl)-[1,2,4]triazolo[1,5-a]pyridin-2-yl)amino)phenyl)piperazin-1-yl)-5-oxopentyl)amino)isoindoline-1,3-dione (JA-196);
2-(2,6-Dioxopiperidin-3-yl)-5-((6-(4-(4-((5-(4-(methylsulfonyl)phenyl)-[1,2,4]triazolo[1,5-a]pyridin-2-yl)amino)phenyl)piperazin-1-yl)-6-oxohexyl)amino)isoindoline-1,3-dione (JA-198);
2-(2,6-Dioxopiperidin-3-yl)-5-((7-(4-(4-((5-(4-(methylsulfonyl)phenyl)-[1,2,4]triazolo[1,5-a]pyridin-2-yl)amino)phenyl)piperazin-1-yl)-7-oxoheptyl)amino)isoindoline-1,3-dione (JA-199);
2-(2,6-dioxopiperidin-3-yl)-5-((3-(4-(4-((5-(4-(methylsulfonyl)phenyl)-[1,2,4]triazolo[1,5-a]pyridin-2-yl)amino)phenyl)piperidin-1-yl)-3-oxopropyl)amino)isoindoline-1,3-dione (JA-202);
2-(2,6-dioxopiperidin-3-yl)-5-((8-(4-(4-((5-(4-(methylsulfonyl)phenyl)-[1,2,4]triazolo[1,5-a]pyridin-2-yl)amino)phenyl)piperidin-1-yl)-8-oxooctyl)amino)isoindoline-1,3-dione (JA-203);
N-(8-((2-(2,6-dioxopiperidin-3-yl)-1,3-dioxoisoindolin-5-yl)amino)octyl)-2-(4-(4-((5-(4-(methylsulfonyl)phenyl)-[1,2,4]triazolo[1,5-a]pyridin-2-yl)amino)phenyl)piperidin-1-yl)acetamide (JA-213);
N-(7-((2-(2,6-dioxopiperidin-3-yl)-1,3-dioxoisoindolin-5-yl)amino)heptyl)-2-(4-(4-((5-(4-(methylsulfonyl)phenyl)-[1,2,4]triazolo[1,5-a]pyridin-2-yl)amino)phenyl)piperidin-1-yl)acetamide (JA-214);
2-(2,6-dioxopiperidin-3-yl)-5-(7-(4-(4-((5-(4-(methylsulfonyl)phenyl)-[1,2,4]triazolo[1,5-a]pyridin-2-yl)amino)phenyl)piperazin-1-yl)hept-1-yn-1-yl)isoindoline-1,3-dione (JA-252);
3-(5-((6-(4-(4-((5-(4-(methylsulfonyl)phenyl)-[1,2,4]triazolo[1,5-a]pyridin-2-yl)amino)phenyl)piperazin-1-yl)-6-oxohexyl)oxy)-1-oxoisoindolin-2-yl)piperidine-2,6-dione (JA-261);
3-(5-((6-(4-(4-(8-(3,5-difluoro-4-(morpholinomethyl)phenyl)quinoxalin-2-yl)-1H-pyrazol-1-yl)piperidin-1-yl)-6-oxohexyl)oxy)-1-oxoisoindolin-2-yl)piperidine-2,6-dione (JA-263);
5-(7-(4-(4-(8-(3,5-difluoro-4-(morpholinomethyl)phenyl)quinoxalin-2-yl)-1H-pyrazol-1-yl)piperidin-1-yl)hept-1-yn-1-yl)-2-(2,6-dioxopiperidin-3-yl)isoindoline-1,3-dione (JA-264);
3-(6-(7-(4-(4-((5-(4-(methylsulfonyl)phenyl)-[1,2,4]triazolo[1,5-a]pyridin-2-yl)amino)phenyl)piperazin-1-yl)-7-oxoheptyl)-1-oxoisoindolin-2-yl)piperidine-2,6-dione (JA-268);
3-(6-(7-(4-(4-(8-(3,5-difluoro-4-(morpholinomethyl)phenyl)quinoxalin-2-yl)-1H-pyrazol-1-yl)piperidin-1-yl)-7-oxoheptyl)-1-oxoisoindolin-2-yl)piperidine-2,6-dione (JA-269); and
3-(5-((6-(4-(4-(8-(3,5-difluoro-4-(morpholinomethyl)phenyl)quinoxalin-2-yl)-1H-pyrazol-1-yl)piperidin-1-yl)-6-oxohexyl)amino)-1-oxoisoindolin-2-yl)piperidine-2,6-dione (JA-273);
or a pharmaceutically acceptable salt or analog thereof.

80. A composition comprising a heterobifunctional compound according to claim 1 or a pharmaceutically acceptable salt or analog thereof.

81. A method of treating a JAK-mediated disease, GSTP1-mediated disease, or JAK- and GSTP1-mediated disease, comprising administering to a subject with a JAK-mediated disease a heterobifunctional compound or a pharmaceutically acceptable salt or analog thereof according to claim 1.

82. The method of claim 81, wherein the JAK-mediated disease is selected from the group consisting of the JAK-mediated cancer, the JAK-mediated inflammatory disorders, the JAK-mediated auto-immune diseases, the JAK-mediated dermatological disorders, the JAK-mediated viral infections, the JAK-mediated dry eye disorders, the JAK-mediated bone remodeling disorders, and the JAK-mediated organ transplant associated immunological complications.

83-87. (canceled)

88. The method of claim 82, wherein
the JAK-mediated cancer is selected from the group consisting of brain cancer, stomach cancer, gastrointestinal tract cancer, liver cancer, biliary passage cancer, breast cancer, ovary cancer, cervix cancer, prostate cancer, testis cancer, penile cancer, genitourinary tract cancer, esophagus cancer, larynx cancer, skin cancer, lung cancer, pancreas cancer, thyroid cancer, gland cancer, bladder cancer, kidney cancer, muscle cancer, bone cancer, cancers of the hematopoietic system, myeloproliferative neoplasms, essential thrombocythemia, polycythemia vera, primary myelofibrosis, chronic neutrophilic leukemia, acute lymphoblastic leukemia, Hodgkin's lymphoma, chronic myelomonocytic leukemia, systemic mast cell disease, hypereosinophilic syndrome, cutaneous T-cell lymphoma, B-cell lymphoma, and myeloma; and/or
the JAK-mediated inflammatory disorders are selected from the group consisting of ankylosing spondylitis, Crohn's disease, inflammatory bowel disease, ulcerative colitis, and ischemia reperfusion injuries; and/or
the JAK-mediated auto-immune diseases are selected from the group consisting of multiple sclerosis, rheumatoid arthritis, psoriatic arthritis, juvenile idiopathic arthritis, psoriasis, myasthenia gravis, type I diabetes, systemic lupus erythematosus, IgA nephropathy, autoimmune thyroid disorders, alopecia areata, and bullous pemphigoid; and/or
the JAK-mediated dermatological disorders are selected from the group consisting of atopic dermatitis, pruritus, alopecia areata, psoriasis, skin rash, skin irritation, skin sensitization, chronic mucocutaneous candidiasis, dermatomyositis, erythema multiforme, palmoplantar pustulosis, vitiligo, polyarteritis nodosa, and STING-associated vasculopathy; and/or
the JAK-mediated viral infections are selected from the group consisting of infections of Hepatitis B, Hepatitis C, Human Immunodeficiency Virus (HIV), Human T-lymphotropic Virus (HTLV1), Epstein Barr Virus (EBV), Varicella-Zoster Virus (VZV) and Human Papilloma Virus (HPV); and/or
the JAK-mediated dry eye disorders are selected from the group consisting of dry eye syndrome (DES) and keratoconjunctivitis sicca (KCS); and/or
the JAK-mediated bone remodeling disorders are selected from the group consisting of osteoporosis and osteoarthritis; and/or
the JAK-mediated organ transplant associated immunological complications are selected from the group consisting of graft-versus-host diseases.

89-102. (canceled)

103. The heterobifunctional compound of claim 57, wherein the degradation tag is a moiety selected from the group consisting of FORMULAE 5B, and 5C.

104. The heterobifunctional compound of claim 103, wherein Z is selected from CH₂, C≡C, NH and O.
